(19) United States
(12) Patent Application Publication
(10) Pub. No.: US 2025/0255857 A1
(43) Pub. Date: Aug. 14, 2025

(54) METHODS OF TREATING ANDROGEN RECEPTOR-INDEPENDENT PROSTATE CANCER

(71) Applicant: FOGHORN THERAPEUTICS INC., Cambridge, MA (US)

(72) Inventors: Salonee PARIKH, Needham, MA (US); Brandon ANTONAKOS, West Roxbury, MA (US); Huawei CHEN, Carlisle, MA (US); Qianhe ZHOU, Winchester, MA (US); Ammar ADAM, Cambridge, MA (US)

(21) Appl. No.: 18/856,340

(22) PCT Filed: Apr. 11, 2023
(86) PCT No.: PCT/US2023/018195

§ 371 Date: Oct. 11, 2024

Related U.S. Application Data
(60) Provisional application No. 63/329,876, filed on Apr. 11, 2022.

(51) Int. Cl. A61K 31/4545; A61K 31/4166; A61K 31/573; A61K 39/395; A61P 35/00

(52) U.S. Cl. CPC A61K 31/4545; A61K 31/4166; A61K 31/573; A61K 39/3955; A61P 35/00

(57) ABSTRACT

The present invention relates to methods and compositions for the treatment of BAF-related disorders such as cancer.

### BACKGROUND

Disorders can be affected by the BAF complex. BRD9 is a component of the BAF complex. The present invention relates to useful methods and compositions for the treatment of BAF-related disorders, such as cancer.

### SUMMARY OF THE INVENTION

Bromodomain-containing protein 9 (BRD9) is a protein encoded by the BRD9 gene on chromosome 5. BRD9 is a component of the BAF (BRG1- or BRM-associated factors) complex, a SWI/SNF ATPase chromatin remodeling complex, and belongs to family IV of the bromodomain-containing proteins. BRD9 is present in several SWI/SNF ATPase chromatin remodeling complexes and is upregulated in multiple cancer cell lines. Accordingly, agents that reduce the levels and/or activity of BRD9 may provide new methods for the treatment of disease and disorders, such as cancer and infection. The inventors have found that depleting BRD9 in several varying prostate cancer cell lines has a positive effect in decreasing tumor growth volume while maintaining tolerability. Thus, agents that degrade BRD9 (e.g., compounds) are useful in the treatment of disorders (e.g., cancers or infections) related to BAF and/or BRD9

Without wishing to be bound by theory, it is believed that depleting or inhibiting BRD9 results in the treatment of androgen receptor-independent prostate cancers.

In an aspect, the present disclosure features a method of treating androgen receptor-independent prostate cancer in a subject in need thereof, the method including the step of administering to the subject an effective amount of a compound that reduces the level and/or activity of BRD9, or a pharmaceutically acceptable salt thereof.

In an aspect, the present disclosure features a method of slowing progression of androgen receptor-independent prostate cancer in a subject in need thereof, the method including the step of administering to the subject an effective amount of a compound that reduces the level and/or activity of BRD9, or a pharmaceutically acceptable salt thereof.

In an aspect, the present disclosure features a method of reducing recurrence of androgen receptor-independent prostate cancer in a subject in need thereof, the method including the step of administering to the subject an effective amount of a compound that reduces the level and/or activity of BRD9, or a pharmaceutically acceptable salt thereof.

In an aspect, the present disclosure features a method of decreasing the rate of metastatic tumor seeding of androgen receptor-independent prostate cancer in a subject in need thereof, the method including the step of administering to the subject an effective amount of a compound that reduces the level and/or activity of BRD9, or a pharmaceutically acceptable salt thereof.

In an aspect, the present disclosure features a method of decreasing metastatic tumor nodule formation of androgen receptor-independent prostate cancer in a subject in need thereof, the method including the step of administering to the subject an effective amount of a compound that reduces the level and/or activity of BRD9, or a pharmaceutically acceptable salt thereof.

In an aspect, the present disclosure features a method of decreasing the spread of metastatic tumor nodule formation of androgen receptor-independent prostate cancer in a subject in need thereof, the method including the step of administering to the subject an effective amount of a compound that reduces the level and/or activity of BRD9, or a pharmaceutically acceptable salt thereof.

In an aspect, the present disclosure features a method of decreasing metastatic colonization of androgen receptor-independent prostate cancer in a subject in need thereof, the method including the step of administering to the subject an effective amount of a compound that reduces the level and/or activity of BRD9, or a pharmaceutically acceptable salt thereof.

In some embodiments, the androgen receptor-independent prostate cancer has failed to respond a previous treatment with an anti-cancer therapy.

In an aspect, the present disclosure features a method of treating prostate cancer that has failed to respond to a previous treatment with an anti-cancer therapy in a subject in need thereof, the method including the step of administering to the subject an effective amount of a compound that reduces the level and/or activity of BRD9, or a pharmaceutically acceptable salt thereof. In some embodiments, the prostate cancer is neuroendocrine prostate cancer.

In an aspect, the present disclosure features a method of treating neuroendocrine prostate cancer in a subject in need thereof, the method including the step of administering to the subject an effective amount of a compound that reduces the level and/or activity of BRD9, or a pharmaceutically acceptable salt thereof.

In some embodiments, the neuroendocrine prostate cancer is androgen receptor-independent. In some embodiments, the neuroendocrine prostate cancer has failed to respond to a previous treatment with an anti-cancer therapy.

In some embodiments, the compound is 3-(6-(7-((1-(4-(6-(azetidin-1-yl)-2-methyl-1-oxo-1,2-dihydro-2,7-naphthyridin-4-yl)-2,6-dimethoxybenzyl) piperidin-4-yl)methyl)-2,7-diazaspiro[3.5]nonan-2-yl)-1-oxoisoindolin-2-yl) piperidine-2,6-dione. having the structure:

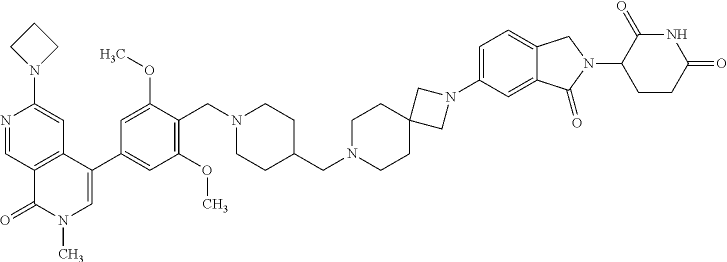 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1]

or a pharmaceutically acceptable salt thereof.

In some embodiments of any of the aspects disclosed herein, the effective amount of the compound or a pharmaceutically acceptable salt thereof, is administered in a dose of 20-120 mg/kg (e.g., 20-60 mg/kg, 20-40 mg/kg, 40-80 mg/kg, 40-60 mg/kg, 60-80 mg/kg, or 80-120 mg/kg).

In some embodiments of any of the aspects disclosed herein, the effective amount of the compound or a pharmaceutically acceptable salt thereof, is administered in a dose of 20 mg/kg. In some embodiments of any of the aspects disclosed herein, the effective amount of the compound or a pharmaceutically acceptable salt thereof, is administered in a dose of 40 mg/kg. In some embodiments of any of the aspects disclosed herein, the effective amount of the compound or a pharmaceutically acceptable salt thereof, is administered in a dose of 50 mg/kg. In some embodiments of any of the aspects disclosed herein, the effective amount of the compound or a pharmaceutically acceptable salt thereof, is administered in a dose of 60 mg/kg. In some embodiments of any of the aspects disclosed herein, the effective amount of the compound or a pharmaceutically acceptable salt thereof, is administered in a dose of 80 mg/kg. In some embodiments of any of the aspects disclosed herein, the effective amount of the compound or a pharmaceutically acceptable salt thereof, is administered in a dose of 120 mg/kg.

In some embodiments of any of the aspects disclosed herein, the effective amount of the compound or a pharmaceutically acceptable salt thereof, is administered to the subject at least once per week (e.g., once per week).

In some embodiments of any of the aspects disclosed herein, the effective amount of the compound or a pharmaceutically acceptable salt thereof, is administered to the subject at least twice per week (e.g., twice per week).

In some embodiments of any of the aspects disclosed herein, the effective amount of the compound or a pharmaceutically acceptable salt thereof, is administered to the subject at least once per week.

In some embodiments of any of the aspects disclosed herein, the effective amount of the compound or a pharmaceutically acceptable salt thereof, is administered to the subject at least twice per week.

In some embodiments of any of the aspects disclosed herein, the effective amount of the compound or a pharmaceutically acceptable salt thereof, is administered in a dose of 20 mg/kg once per week. In some embodiments of any of the aspects disclosed herein, the effective amount of the compound or a pharmaceutically acceptable salt thereof, is administered in a dose of 20 mg/kg twice per week.

In some embodiments of any of the aspects disclosed herein, the effective amount of the compound or a pharmaceutically acceptable salt thereof, is administered in a dose of 40 mg/kg once per week. In some embodiments of any of the aspects disclosed herein, the effective amount of the compound or a pharmaceutically acceptable salt thereof, is administered in a dose of 40 mg/kg twice per week.

In some embodiments of any of the aspects disclosed herein, the effective amount of the compound or a pharmaceutically acceptable salt thereof, is administered in a dose of 50 mg/kg once per week. In some embodiments of any of the aspects disclosed herein, the effective amount of the compound or a pharmaceutically acceptable salt thereof, is administered in a dose of 50 mg/kg twice per week.

In some embodiments of any of the aspects disclosed herein, the effective amount of the compound or a pharmaceutically acceptable salt thereof, is administered in a dose of 60 mg/kg once per week. In some embodiments of any of the aspects disclosed herein, the effective amount of the compound or a pharmaceutically acceptable salt thereof, is administered in a dose of 60 mg/kg twice per week.

In some embodiments of any of the aspects disclosed herein, the effective amount of the compound or a pharmaceutically acceptable salt thereof, is administered in a dose of 80 mg/kg once per week. In some embodiments of any of the aspects disclosed herein, the effective amount of the compound or a pharmaceutically acceptable salt thereof, is administered in a dose of 80 mg/kg twice per week.

In some embodiments of any of the aspects disclosed herein, the effective amount of the compound or a pharmaceutically acceptable salt thereof, is administered in a dose of 120 mg/kg once per week. In some embodiments of any of the aspects disclosed herein, the effective amount of the compound or a pharmaceutically acceptable salt thereof, is administered in a dose of 120 mg/kg twice per week.

In some embodiments of any of the aspects disclosed herein, the compound or a pharmaceutically acceptable salt thereof is administered to the subject in a 14-day dosing cycle.

In some embodiments of any of the aspects disclosed herein, the compound or a pharmaceutically acceptable salt thereof is administered to the subject in a 21-day dosing cycle.

In some embodiments of any of the aspects disclosed herein, the compound or a pharmaceutically acceptable salt thereof is administered to the subject in a 28-day dosing cycle.

In some embodiments of any of the aspects disclosed herein, the compound or a pharmaceutically acceptable salt thereof, is administered to the subject intravenously.

In some embodiments of any of the aspects disclosed herein, the compound or a pharmaceutically acceptable salt thereof, is administered to the subject subcutaneously.

In some embodiments of any of the aspects disclosed herein, the compound or a pharmaceutically acceptable salt thereof, is administered to the subject intramuscularly.

In some embodiments, the compound is 3-((4-(4-(1-(2,6-dimethoxy-4-(1,4,5-trimethyl-6-oxo-1,6-dihydropyridin-3-yl)benzyl)-3,3-difluoropiperidin-4-yl) piperazin-1-yl)-3-fluorophenyl)amino) piperidine-2,6-dione having the structure:

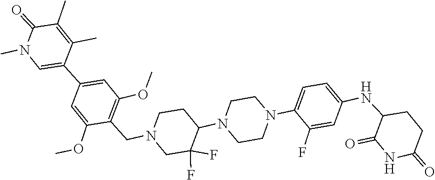 [SMILES: COc1cc(-c2cn(C)c(=O)c(C)c2C)cc(OC)c1CN1CCC(N2CCN(c3ccc(NC4CCC(=O)NC4=O)cc3F)CC2)C(F)(F)C1]

or a pharmaceutically acceptable salt thereof.

In some embodiments, the prostate cancer is castration-resistant prostate cancer (CRPC). In some embodiments, the prostate cancer is small cell prostate cancer.

In some embodiments, the effective amount is an amount sufficient to reduce the level of neuroendocrine prostate cancer cells in the subject compared to a subject that is not administered the compound or pharmaceutically acceptable salt thereof.

In some embodiments, the anti-cancer therapy is active surveillance, surgery, radiation therapy, high-intensity focused ultrasound (HIFU), cryotherapy, hormone therapy, chemotherapy, immunotherapy, vaccine treatment, immune checkpoint inhibitors, targeted therapy drugs, or bone-directed treatment.

In some embodiments, the anti-cancer therapy is abiraterone acetate, alendronate, apalutamide, bicalutamide, cabazitaxel, carboplatin, cisplatin, darolutamide, degarelix, denosumab, docetaxel, enzalutamide, etoposide, flutamide, goserelin acetate, ibandronate, leuprolide acetate, lynparza, mitoxantrone hydrochloride, nilutamide, olaparib, pamidronate, radium 223 dichloride, relugolix, risedronate, rucaparib camsylate, sipuleucel-T, or zoledronic acid, or combinations thereof. In some embodiments, the anti-cancer therapy is enzalutamide.

Ins some embodiments, the subject is further administered at least one additional anti-cancer therapy. In some embodiments, the additional anti-cancer therapy is administered prior to the administering of the compound or pharmaceutically acceptable salt thereof. In some embodiments, the additional anti-cancer therapy is administered in addition to the administering of the compound or pharmaceutically acceptable salt thereof. In some embodiments, the additional anti-cancer therapy is administered subsequent to the administering of the compound or pharmaceutically acceptable salt thereof.

In some embodiments, the subject is further administered a treatment for symptoms of prostate cancer. Nonlimiting examples of symptoms of prostate cancer that may be treated include frequent urination, weak or uninterrupted urine flow or the need to strain to empty the bladder, blood in the urine, a new onset of erectile dysfunction, pain or burning during urination, and discomfort or pain when sitting. In some embodiment, the further treatment is prednisone, methylprednisolone, pembrolizumab, or a combination thereof.

In some embodiments, the effective amount is an amount sufficient to reduce the level of luminal prostate cancer cell to neuroendocrine prostate cancer cell trans-differentiation measured by lower expression levels of CHGA, SYP, and/or ENO2 compared to a subject that is not administered the compound or pharmaceutically acceptable salt thereof.

In some embodiments, the effective amount is an amount sufficient to reduce the level of adenocarcinoma to neuroendocrine trans-differentiation measured by lower expression levels of CHGA, SYP, and/or ENO2 compared to a subject that is not administered the compound or pharmaceutically acceptable salt thereof.

In some embodiments, the effective amount is an amount sufficient to reduce the level of neuroendocrine prostate cancer cells compared to a subject that is not administered the compound or pharmaceutically acceptable salt thereof.

In some embodiments, the prostate cancer has been determined to have or predicted to have lower expression levels of AR, KLK2, KLK3, CDH1, CYLD, NKX3-1, SLC45A3, TARP, PTEN, SPDEF, TP53, or RB1, or combinations thereof compared to a subject that does not have prostate cancer.

In some embodiments, the prostate cancer has been determined to have or predicted to have lower expression levels of AR, KLK2, KLK3, CDH1, CYLD, NKX3-1, SLC45A3, TARP, PTEN, SPDEF, TP53, or RB1, or combinations thereof compared to standard levels for prostate cancer.

In some embodiments, the prostate cancer has been determined to have or predicted to have higher expression levels of CHGB, CHGA, SYP, ENO2, PEG10, SNAP25, SRRM4, VGF, VIM, SCGN, PAPPA2, or WNT11, or combinations thereof compared to a subject that does not have prostate cancer.

In some embodiments, the prostate cancer has been determined to have or predicted to have higher expression levels of CHGB, CHGA, SYP, ENO2, PEG10, SNAP25, SRRM4, VGF, VIM, SCGN, PAPPA2, or WNT11, or combinations thereof compared to standard levels for prostate cancer.

In some embodiments, the prostate cancer has been determined to have or predicted to have higher expression levels of ASCL1, EZH2, DLX5, DLX6, SOX2, NKX2-2, HES6, SOX9, KDM3A, FOXA2, AURKA, MYCN, MYC, AKT, POU3F2/BRN2, NANOG, ONECUT2, or NKX2-1, or combinations thereof compared to a subject that does not have prostate cancer.

In some embodiments, the prostate cancer has been determined to have or predicted to have higher expression levels of ASCL1, EZH2, DLX5, DLX6, SOX2, NKX2-2, HES6, SOX9, KDM3A, FOXA2, AURKA, MYCN, MYC, AKT, POU3F2/BRN2, NANOG, ONECUT2, or NKX2-1, or combinations thereof compared to standard levels for prostate cancer.

In some embodiments, wherein the prostate cancer has been determined to have or predicted to have lower expression levels of RE1 silencing transcription factor (REST).

In some embodiments, the prostate cancer has been determined to have or predicted to have expression of AR and KLK3 (PSA).

In some embodiments, the prostate cancer has been determined to have or predicted to have undetectable expression of CHGA and SYP.

In some embodiments, the prostate cancer has been determined to have or predicted to have expression of AR, KLK3 (PSA), CHGA and SYP.

In some embodiments, the prostate cancer has been determined to have or predicted to have undetectable expression of AR and KLK3 (PSA).

In some embodiments, the prostate cancer has been determined to have or predicted to have expression of CHGA and SYP.

In some embodiments, the prostate cancer has been determined to have or predicted to have undetectable expression of AR, KLK3 (PSA), CHGA and SYP.

In some embodiments, the prostate cancer has been determined or predicted to have a high level of TMPRSS2-ERG fusions.

In some embodiments, the prostate cancer is metastatic.

In some embodiments, expression of BRD9, GLTSCR1, CXXC5 or TET2 is increased in the prostate cancer compared to a subject that does not have prostate cancer.

In some embodiments, expression of BRD9 is increased in the prostate cancer compared to a subject that does not have prostate cancer.

In some embodiments, expression of GLTSCR1 is increased in the prostate cancer compared to a subject that does not have prostate cancer.

In some embodiments, expression of TET2, CXXC5, H3K27ac, ID1, PFN2, or ID3 in the subject is increased in the prostate cancer determined to or predicted to be resistant to enzalutamide compared to a prostate cancer that responds to treatment with enzalutamide.

In some embodiments, the prostate cancer has been determined to have or predicted to have undetectable expression of PTEN.

In some embodiments, the prostate cancer has been determined or predicted to be ERG positive.

In some embodiments, the subject is further administered an inhibitor or degrader of ERG. In some embodiments, the ERG inhibitor or degrader is ERGi-USU (1-[2-Thiazolylazo]-2-naphthol).

In some embodiments, the subject is further administered a degrader of AR. In some embodiments, the AR degrader is bavdegalutamide (ARV-110), ARV-766, or AR-V7.

In some embodiments, wherein the subject is further administered an inhibitor of the JAK-STAT pathway. In some embodiments, the JAK-STAT inhibitor is AG490, AZD1480, AZD4205, baricitinib, dasatinib, fedratinib, filgotinib, itacitnib, lestaurtinib, momelotinib, pacritinib, peficitinib, ruxolitinib, siltuximab, tofacitinib, upadacitinib, or WP1066.

In some embodiments, the subject is further administered an inhibitor of the MAPK pathway. In some embodiments, the MAPK pathway inhibitor is a Farnesyltransferase inhibitor (FTI), Sorafenib, Vemurafenib, PLX8394, Dabrafenib, Ulixertinib, Simvastatin, Alisertib, or Teriflunomide.

In some embodiments, the subject is further administered an inhibitor of the PI3K-AKT-mTOR pathway. In some embodiments, the PIK3-AKT-mTOR inhibitor is everolimus, alpelisib, idelalisib or copanlisib.

In some embodiments, the subject has a PSA level of 4 ng/ml or more prior to the administering of the compound or a pharmaceutically acceptable salt thereof.

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a H or an alkyl group that is attached to a parent molecular group through a carbonyl group, as defined herein, and is exemplified by formyl (i.e., a carboxaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6 carbon atoms, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, and monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to a mono- or polycyclic radical of 5 to 12 atoms having at least one aromatic ring and containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Another exemplary substituent is oxo. For example, a carbonyl group is a carbon (e.g., alkyl carbon, alkenyl carbon, alkynyl carbon, heteroalkyl carbon, heteroalkenyl carbon, heteroalkynyl carbon, carbocyclyl carbon, etc.) substituted with oxo. Alternatively, sulfur may be substituted with one or two oxo groups (e.g., —SO— or —SO₂— within a substituted heteroalkyl, heteroalkenyl, heteroalkynyl, or heterocyclyl group). Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)). In some embodiments, alkyl, alkenyl, alkynyl, heteroalkyl, heteroalkenyl, and heteroalkynyl are optionally substituted with 1, 2, 3, 4, or 5 substituents independently selected from the group consisting of aryl (e.g., substituted and unsubstituted phenyl), carbocyclyl (e.g., substituted and unsubstituted cycloalkyl), halo (e.g., fluoro), hydroxyl, heteroaryl, heterocyclyl, amino (e.g., NH₂ or mono- or dialkyl amino), azido, cyano, nitro, thiol, and oxo. In some embodiments, the substituents are themselves unsubstituted.

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with 3H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent. Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HRBM-associated factors complex in a human cell.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be achieved by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “BRD9” refers to bromodomain-containing protein 9, a component of the BAF (BRG1- or BRM-associated factors) complex, a SWI/SNF ATPase chromatin remodeling complex, and belongs to family IV of the bromodomain-containing proteins. BRD9 is encoded by the BRD9 gene, the nucleic acid sequence of which is set forth in SEQ ID NO: 1. The term “BRD9” also refers to natural variants of the wild-type BRD9 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type BRD9, which is set forth in SEQ ID NO: 2.

As used herein, the term “BRD9-related disorder” refers to a disorder that is caused or affected by the level and/or activity of BRD9.

As used herein, the term “cancer” refers to a condition caused by the proliferation of malignant neoplastic cells, such as tumors, neoplasms, carcinomas, sarcomas, leukemias, and lymphomas.

As used herein, the term “degrader” refers to a small molecule compound including a degradation moiety, wherein the compound interacts with a protein (e.g., BRD9) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRD9. In one example, the moiety binds to a protease or a ubiquitin ligase that metabolizes the protein, e.g., BRD9.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BRD9,” is meant decreasing the level of an activity related to an BRD9, or a related downstream effect. A non-limiting example of inhibition of an activity of BRD9 is decreasing the level of a BAF complex (e.g., GBAF) in a cell. The activity level of BRD9 may be measured using any method known in the art. In some embodiments, an agent which reduces the activity of BRD9 is a small molecule BRD9 inhibitor. In some embodiments, an agent which reduces the activity of BRD9 is a small molecule BRD9 degrader.

By “reducing the level of BRD9,” is meant decreasing the level of BRD9 in a cell or subject. The level of BRD9 may be measured using any method known in the art.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/ml) or percentage relative to total protein or mRNA in a sample.

As used herein, the term “inhibitor” refers to any agent which reduces the level and/or activity of a protein (e.g., BRD9). Non-limiting examples of inhibitors include small molecule inhibitors, degraders, antibodies, enzymes, or polynucleotides (e.g., siRNA).

As used herein, the terms “effective amount,” “therapeutically effective amount,” and “a “sufficient amount” of an agent that reduces the level and/or activity of BRD9 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of BRD9 sufficient to achieve a treatment response as compared to the response obtained without administration of the agent that reduces the level and/or activity of BRD9. The amount of a given agent that reduces the level and/or activity of BRD9 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of BRD9 of the present disclosure is an amount which results in a beneficial or desired result in a subject as compared to a control. As defined herein, a therapeutically effective amount of an agent that reduces the level and/or activity of BRD9 of the present disclosure may be readily determined by one of ordinary skill by routine methods known in the art. Dosage regimen may be adjusted to provide the optimum therapeutic response.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a subject. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein, mRNA, DNA, or gene expression levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

As used herein, the term “undetectable” refers to a measurement and/or observation that is not able to be obtained either due to insufficient presence of the substance in question or complete lack thereof. Instrumentation limitations can contribute to an undetectable measurement.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “adenocarcinoma” refers to a form of cancer that originates in glandular cells.

As used herein, the term “trans-differentiation” refers to the conversion of one a cell type into a second cell type without passing through an intermediate pluripotent state.

As used herein, the term “luminal prostate cancer” refers to prostate cancer that arises in luminal cells.

As used herein, the term “neuroendocrine prostate cancer” refers to a subtype of prostate cancer that exists in neuroendocrine cells located in the prostate cancer.

As used herein, the term “metastatic tumor seeding” refers to a process by which cancerous cells are spread throughout the body and cause the formation of tumors at locations in the body other than the first location in the body where the cancer cells appeared.

As used herein, the term “metastatic tumor nodule formation” refers to the formation of a tumor nodule at a secondary cancer site.

As used herein, the term “metastatic colonization” refers to the process by which cancer cells spread throughout the body from a primary cancer site to one or more secondary cancer sites.

The details of one or more embodiments of the invention are set forth in the description below. Other features, objects, and advantages of the invention will be apparent from the description and from the claims.

### BRIEF DESCRIPTION OF THE DRAWINGS

FIG. 1A is a graph illustrating inhibition of tumor growth in mice engrafted with LNCaP prostate xenograft tumors and treated with Compound 1, enzalutamide and the combination of both compounds.

FIG. 1B is a graph showing the bodyweight change of the animals over the course of the study.

FIG. 2A is a graph illustrating inhibition of tumor growth in mice engrafted with VCaP prostate xenograft tumors and treated with Compound 1, enzalutamide and the combination of both compounds.

FIG. 2B is a graph showing bodyweight change of the animals over the course of the study.

FIG. 3A is a graph illustrating inhibition of tumor growth in mice engrafted with PC3 prostate xenograft tumors and treated with Compound 1.

FIG. 3B is a graph showing bodyweight change of the animals over the course of the study.

FIG. 4A is a graph illustrating inhibition of tumor growth in mice engrafted with NCI-H660 prostate xenograft tumors and treated with Compound 1.

FIG. 4B is a graph showing bodyweight change of the animals over the course of the study.

FIG. 5A is a graph illustrating inhibition of tumor growth in mice engrafted with C₄₂B prostate xenograft tumors and treated with Compound 1 or enzalutamide.

FIG. 5B is a graph showing bodyweight change of the animals over the course of the study.

FIG. 6A is a graph illustrating inhibition of tumor growth in mice engrafted with 22Rv1 prostate xenograft tumors and treated with Compound 1, enzalutamide and the combination of both compounds.

FIG. 6B is a graph showing bodyweight change of the animals over the course of the study.

FIG. 7A is a graph illustrating inhibition of tumor growth in mice engrafted with DU145 prostate xenograft tumors and treated with Compound 1.

FIG. 7B is a graph showing bodyweight change of the animals over the course of the study.

FIG. 8 is a graph showing plasma concentration over time for Compound 1 in the DU145 PK/PD study.

FIG. 9 is a graph illustrating a positive correlation between plasma PK and tumor PD for the DU145 PK/PD study.

FIG. 10A is a graph illustrating inhibition of tumor growth in mice engrafted with VCaP prostate xenograft tumors and treated with Compound 1, enzalutamide and the combination of both compounds.

FIG. 10B is a graph showing bodyweight change of the animals over the course of the study.

### DETAILED DESCRIPTION

The progression of prostate cancer typically depends on activity of the androgen receptor, a transcription factor that effectuates changes in gene expression upon binding to androgenic hormones such as testosterone and dihydroxytestosterone. Accordingly, existing treatments for prostate cancer include androgen starvation, e.g., reduction in androgen levels caused by surgical or chemical castration, or the administration of androgen receptor signaling inhibitors. However, subtypes of prostate cancer such as castration-resistant prostate cancer (CRPC) can grow and spread even with very low levels of androgen, and resistance typically develops to androgen receptor signaling inhibitors. The present inventors have discovered that forms of prostate cancer that grow and spread independently of the androgen receptor may be treated with compounds that reduce the level and/or activity of bromodomain containing protein 9 (BRD9). Accordingly, the present invention features methods of treating androgen receptor-independent prostate cancer with compounds that reduce the level and/or activity of BRD9.

### BRD9 Inhibitors

Compounds that reduce the level of an activity related to BRD9, or a related downstream effect, or reduce the level of BRD9 in a cell or subject, may be used in accordance with the methods of the invention. BRD9 inhibitors are disclosed in US20220098190, US20220048906, US20210230190, US 20210009568, US20190247509, US20180044335, WO 2020051235, WO 2020160192, WO 2020160193, WO 2020160198, WO 2021055295, and WO 2021178920, the BRD9 inhibitors of which are incorporated by reference into the present application. In some embodiments of the method of the invention, the BRD9 inhibitors are BRD9 degraders.

In a preferred embodiment of the methods of the present invention, the BRD9 inhibitor has the structure of Compound 1, or a pharmaceutically acceptable salt thereof.

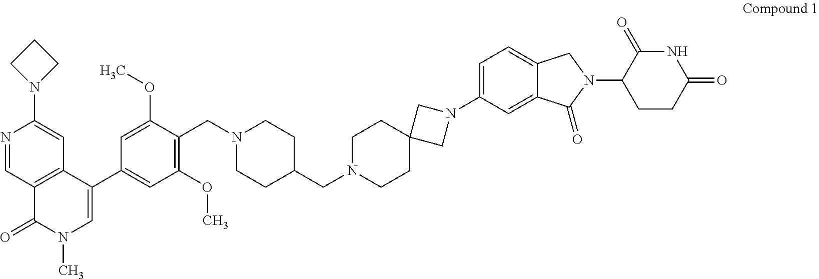 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1]

Methods for synthesizing compound 1 are disclosed in US 2021-0230190 A1, the synthetic methods of which are incorporated by reference into the present application.

The BRD9 inhibitor may be, e.g., a compound of Formula I:

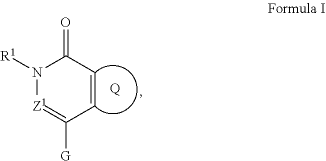

where

R¹ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
Z¹ is CR² or N;
R² is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, or optionally substituted C₂-C₉ heteroaryl;

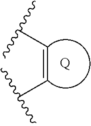

is

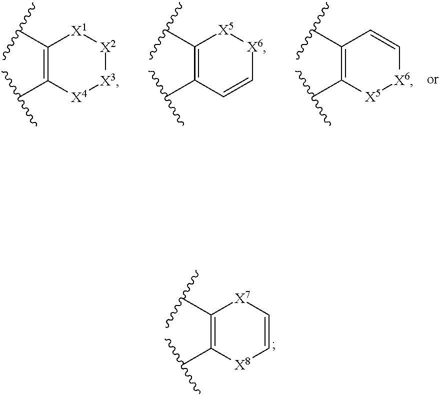

X¹ is a bond, O, NR^{3a},

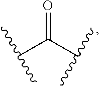 [SMILES: *C(*)=O]

or CR^{4a}R^{5a};
X² is O, NR^{3b},

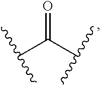 [SMILES: *C(*)=O]

or CR^{4b}R^{5b};
X³ is O, NR^{3c},

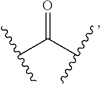 [SMILES: *C(*)=O]

or CR^{4c}R^{5c};
X⁴ is a bond, O, NR^{3d},

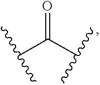 [SMILES: *C(*)=O]

or CR^{4d}R^{5d};
X⁵ is O or NR^{3e} and X⁶ is CR^{4f}R^{5f}, or X⁵ is CR^{4e}R^{5e} and X⁶ is O or NR^{3f};
X⁷ is O, NR^{3g}, or CR^{4g}R^{5g};
X⁸ is O, NR^{3h}, or CR^{4h}R^{5h};
each of R^{3a}, R^{3b}, R^{3c}, and R^{3d} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted C₁-C₆ acyl, thiol, optionally substituted sulfone, optionally substituted sulfonamide, or optionally substituted amino, or R^{3a} and R^{4b}, R^{4a} and R^{3b}, R^{4b} and R^{4a}, R^{3b} and R^{4c}, R^{4b} and R^{4c}, R^{3c} and R^{4b}, R^{3c} and R^{4d}, R^{4c} and R^{4d}, and/or R^{3d} and R^{4c}, together with the atoms to which each is attached, combine to form optionally substituted C₂-C₉ heterocyclyl;
each of R^{4a}, R^{4b}, R^{4c}, and R^{4d} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted C₁-C₆ acyl, thiol, optionally substituted sulfone, or optionally substituted amino, or R^{3a} and R^{4b}, R^{4a} and R^{3b}, R^{4b} and R^{4a}, R^{3b} and R^{4c}, R^{4b} and R^{4c}, R^{3c} and R^{4b}, R^{3c} and R^{4d}, R^{4c} and R^{4d}, and/or R^{3d} and R^{4c}, together with the atoms to which each is attached, combine to form optionally substituted C₂-C₉ heterocyclyl;
   each of R^{5a}, R^{5b}, R^{5c}, and R^{5d} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino;
each of R^{3e}, R^{3f}, R^{3g}, and R^{3h} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted C₁-C₆ acyl, thiol, optionally substituted sulfone, optionally substituted sulfonamide, or optionally substituted amino, or R^{3e} and R^{4f} or R^{4e} and R^{3f}, together with the atoms to which each is attached, combine to form optionally substituted heterocyclycl;
each of R^{4e}, R^{4f}, R^{4g}, and R^{4h} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted C₁-C₆ acyl, thiol, optionally substituted sulfone, optionally substituted sulfonamide, or optionally substituted amino, or R^{3e} and R^{4f} or R^{4e} and R^{3f}, together with the atoms to which each is attached, combine to form optionally substituted heterocyclycl;
each of R^{5e}, R^{5f}, R^{5g}, and R^{5h} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; and
G is optionally substituted C₆-C₁₀ aryl, optionally substituted C₃-C₁₀ carbocyclyl, optionally substituted C₂-C₉ heteroaryl, or C₂-C₉ heterocyclyl, or a pharmaceutically acceptable salt thereof.

In some embodiments,

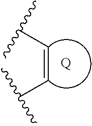

is

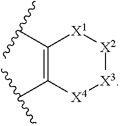

In some embodiments,

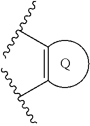

is

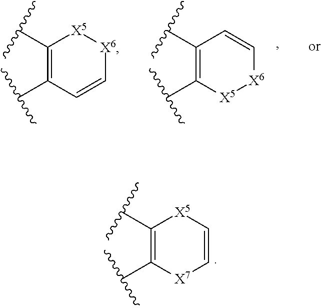

In some embodiments,

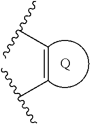

is

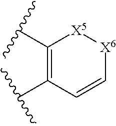

or

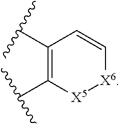

In some embodiments,

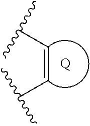

is

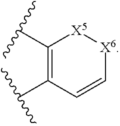

The BRD9 inhibitor may be, e.g., a compound of Formula I:

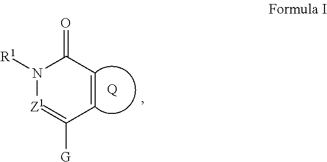

where
R¹ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
Z¹ is CR² or N;
R² is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, or optionally substituted C₂-C₉ heteroaryl;

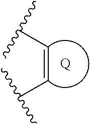

is

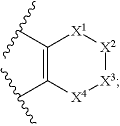

X¹ is a bond, O, NR^{3a}, or CR^{4a}R^{5a};
X² is O, NR^{3b}, or CR^{4b}R^{5b};
X³ is O, NR^{3c}, or CR^{4c}R^{5c};
X⁴ is a bond, O, NR^{3d}, or CR^{4d}R^{5d};
each of R^{3a}, R^{3b}, R^{3c}, and R^{3d} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted C₁-C₆ acyl, thiol, optionally substituted sulfone, optionally substituted sulfonamide, or optionally substituted amino, or R^{3a} and R^{4b}, R^{4a} and R^{3b}, R^{4b} and R^{4a}, R^{3b} and R^{4c}, R^{4b} and R^{4c}, R^{3c} and R^{4b}, R^{3c} and R^{4d}, R^{4c} and R^{4d}, and/or R^{3d} and R^{4c}, together with the atoms to which each is attached, combine to form optionally substituted C₂-C₉ heterocyclyl;
each of R^{4a}, R^{4b}, R^{4c}, and R^{4d} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted C₁-C₆ acyl, thiol, optionally substituted sulfone, or optionally substituted amino, or R^{3a} and R^{4b}, R^{4a} and R^{3b}, R^{4b} and R^{4a}, R^{3b} and R^{4c}, R^{4b} and R^{4c}, R^{3c} and R^{4b}, R^{3c} and R^{4d}, R^{4c} and R^{4d}, and/or R^{3d} and R^{4c}, together with the atoms to which each is attached, combine to form optionally substituted C₂-C₉ heterocyclyl;
each of R^{5a}, R^{5b}, R^{5c}, and R^{5d} is, independently, H, halogen, hydroxyl, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; and G is optionally substituted C₆-C₁₀ aryl, optionally substituted C₃-C₁₀ carbocyclyl, optionally substituted C₂-C₉ heteroaryl, or C₂-C₉ heterocyclyl, or a pharmaceutically acceptable salt thereof.

The BRD9 inhibitor may be, e.g., a compound of Formula II:

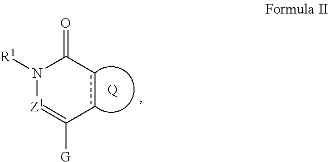

where
R¹ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
Z¹ is CR² or N;
R² is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, or optionally substituted C₂-C₉ heteroaryl;

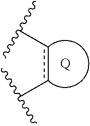

is

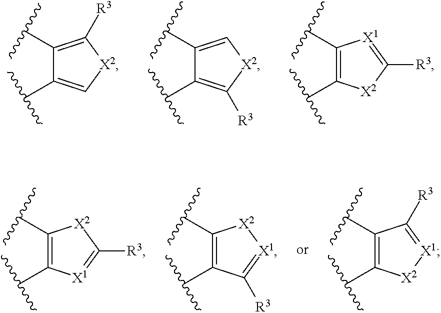

X¹ is CR^{X1} or N;
X² is O or S;
R^{X1} is H or optionally substituted C₁-C₆ alkyl;
R³ is H, cyano, optionally substituted C₁-C₆ alkyl, optionally substituted C₁-C₆ heteroalkyl, optionally substituted C₁-C₆ alkoxy, optionally substituted amino, optionally substituted C₃-C₁₀ carbocyclyl, optionally substituted C₆-C₁₀ aryl, optionally substituted C₃-C₁₀ heterocyclyl, or optionally substituted C₂-C₉ heteroaryl; and
G is optionally substituted C₃-C₁₀ carbocyclyl, C₂-C₉ heterocyclyl, optionally substituted C₆-C₁₀ aryl, or optionally substituted C₂-C₉ heteroaryl, or a pharmaceutically acceptable salt thereof.

The BRD9 inhibitor may be, e.g., a compound of Formula III:

A-L-B Formula III,
where
A is a BRD9 binding moiety;
B is a degradation moiety; and
L has the structure of Formula II:

A¹-(E¹)-(F¹)—(C³)ₘ-(E³)ₙ-(F²)ₒ₁—(F³)ₒ₂-(E²)ₚ-A², Formula IIIA
where
A¹ is a bond between the linker and A;
A² is a bond between B and the linker;
each of m, n, o1, o2, and p is, independently, 0 or 1;
each of E¹ and E² is, independently, O, S, NR^{N}, optionally substituted C₁₋₁₀ alkylene, optionally substituted C₂₋₁₀ alkenylene, optionally substituted C₂₋₁₀ alkynylene, optionally substituted C₂-C₁₀ polyethylene glycol, or optionally substituted C₁₋₁₀ heteroalkylene;
E³ is optionally substituted C₁-C₆ alkylene, optionally substituted C₁-C₆ heteroalkylene, O, S, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆-12 aryl, or optionally substituted C₁₋₇ heteroalkyl;
C₃ is carbonyl, thiocarbonyl, sulphonyl, or phosphoryl; and
each of F¹, F², and F³ is, independently, optionally substituted C₃-C₁₀ carbocyclylene, optionally substituted C₂₋₁₀ heterocyclylene, optionally substituted C₆-C₁₀ arylene, or optionally substituted C₂-C₉ heteroarylene,
or a pharmaceutically acceptable salt thereof.

In some embodiments, the linker has the structure of Formula IIA-a:

A¹-(E¹)-(F¹)—(C³)^{m}-(E²)ₚ-A². Formula IIA-a

In some embodiments, the linker has the structure of Formula IIA-b:

A¹-(E¹)-(F¹)-(E²)ₚ-A². Formula IIA-b

In some embodiments, the linker has the structure of Formula IIA-c:

A¹-(E¹)-(F¹)-A². Formula IIA-c

In some embodiments, the linker has the structure of Formula IIA-d:

A¹-(E¹)-(F¹)—(C³)ₘ—(F²)ₒ₁-A². Formula IIA-d

In some embodiments, the linker has the structure of Formula IIA-e:

A¹-(E¹)-(F¹)-(E³)ₙ-(F²)ₒ₁-(E²)ₚ-A². Formula IIA-e

In some embodiments, the linker has the structure of Formula IIA-f:

A¹-(E¹)-(F¹)—(C³)ₘ-(E³)ₙ-(F²)ₒ₁-(E²)ₚ-A². Formula IIA-f

In some embodiments, the linker has the structure of Formula IIA-g:

A¹-(E¹)-(F¹)-(E³)ₙ-(F²)ₒ₁-A, Formula IIA-g

The BRD9 inhibitor may be, e.g., a compound of Formula IV:

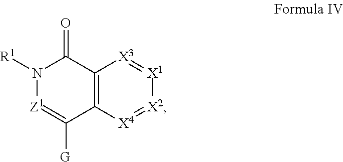

where
R¹ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
Z¹ is CR² or N;
R² is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, or optionally substituted C₂-C₉ heteroaryl;
X¹ is N or CH, and X² is C—R⁷; or X¹ is C—R⁷, and X² is N or CH;
R⁷ is optionally substituted C₁-C₆ alkyl, optionally substituted C₁-C₆ heteroalkyl, optionally substituted C₁-C₆ alkoxy, optionally substituted amino, optionally substituted sulfone, optionally substituted sulfonamide, optionally substituted carbocyclyl having 3 to 6 atoms, or optionally substituted heterocyclyl having 3 to 6 atoms;
X³ is N or CH;
X⁴ is N or CH;
G is optionally substituted C₃-C₁₀ carbocyclyl, C₂-C₉ heterocyclyl, optionally substituted C₆-C₁₀ aryl, or optionally substituted C₂-C₉ heteroaryl, or a pharmaceutically acceptable salt thereof.

The BRD9 inhibitor may be, e.g., a compound of Formula V:

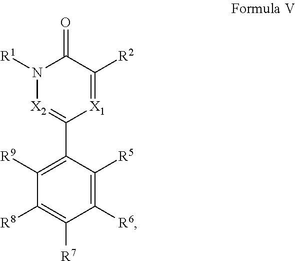

wherein,
R¹ is —C₁₋₃alkyl or -cyclopropyl;
R² is selected from halogen, —C₁₋₃alkyl, —C₁₋₃haloalkyl, —NH₂, —NHC₁₋₃alkyl and —OH,
X₁ is N or CR³
X² is N or CR⁴
wherein X₁ and X₂ cannot be both N in the same molecule
   R³ is H or —C₁₋₃alkyl;
   R⁴ is H or —C₁₋₃alkyl;
   wherein R³ and R⁴ cannot be both —C₁₋₃alkyl in the same molecule;
   alternatively, R² and R³ taken together form a benzene ring or a 5-6 membered heteroarene ring, each of which rings can be optionally and independently substituted with one or more groups selected from halogen, —OH, —NH₂, —NH—C₁₋₃alkyl and —C₁₋₃alkyl, wherein the —C₁₋₃alkyl group can be optionally substituted with 5-6 membered heteroaryl or phenyl,
   R⁵ and R⁹ can be the same or different and are independently selected from —H, —O—C₁₋₃alkyl and —C₁₋₃alkyl;
   R⁶ and R⁸ can be the same or different and are independently selected from —H, —OH, halogen, —NH₂, —C₁₋₃alkyl, —O—C₁₋₃alkyl, —O—C₁₋₃haloalkyl, —C₁₋₃alkyl-O—C₁₋₃alkyl, 4-7 membered heterocycloalkyl, —C₁₋₃alkyl-SO₂—C₁₋₃alkyl, —C₁₋₃alkyl-NH₂, —C₁₋₃alkyl-N(—C₁₋₃alky)₂, —N(C₁₋₃alkyl)₂, —NH—R¹³;
   R¹³ is selected from —SO₂—C₁₋₃alkyl and —C₁₋₃alkyl, wherein the —C₁₋₃alkyl groups can be optionally substituted with 5 to 6 membered heteroaryl;
   alternatively, R⁵ and R⁶ taken together form a benzene ring;
   alternatively, R⁷ and R⁶ or R⁷ and R⁸ taken together form a 5-7 membered heterocycloalkyl optionally substituted with —C₁₋₃alkyl;
   R⁷ is selected from —H, —NH₂, —Y—R¹², —C₁₋₃alkyl and 4-7 membered heterocycloalkyl;
   Y is selected from —CR¹⁰R¹¹—, —SO₂— and —CO—;
   R¹⁰ and R¹¹ can be the same or different and are independently selected from —H or —C₁₋₃alkyl; or R¹⁰ and R¹¹ taken together form a —C₃₋₄cycloalkyl,
   R¹² is selected from —NH₂, —OH, —C₁₋₃alkyl, —N(R¹⁵, R¹⁶), —O—R¹⁷, aryl, 5-6 membered heteroaryl, wherein the aryl or heteroaryl is optionally and independently substituted with one or more halogen, 4-7 membered heterocycloalkyl, which heterocycloalkyl is optionally and independently substituted with one or more groups selected from halogen, —OH, —NH₂, —C₁₋₃alkyl, —NHC₁₋₃alkyl, —N(C₁₋₃alkyl)₂, —O—C₁₋₃alkyl and —CH₂—R¹⁴;
   R¹⁴ is selected from 5-10 membered mono- or bicyclic aryl or heteroaryl, which is optionally substituted with —NH₂, —OH, halogen, —CN, —C₁₋₃alkyl, —O—C₁₋₃alkyl;
   R¹⁵ is —H or —C₁₋₃alkyl;
   R¹⁶ is selected from —C₁₋₃alkyl, —C₂₋₃alkyl-N(C₁₋₃alkyl)₂, —C₁₋₃alkyl-NHC₁₋₃alkyl and 4-7 membered heterocycloalkyl, which heterocycloalkyl is optionally substituted with —C₁₋₃alkyl;
   R¹⁷ is C₁₋₃alkyl or 4-7 membered heterocycloalkyl, which heterocycloalkyl is optionally substituted with C₁₋₃alkyl;
   wherein when R⁷ is Y—R¹², R⁶ and R⁸ can be the same or different and are independently selected from —H, —OH, halogen, —NH₂, —CN, —C₁₋₃alkyl, —C₁₋₃haloalkyl, —O—C₁₋₃alkyl, —O—C₁₋₃haloalkyl and —C₁₋₃alkyl-O—C₁₋₃alkyl;
   wherein at least one of the substituents R⁵ to R⁹ is not hydrogen;
or a pharmaceutically acceptable salt thereof.

The BRD9 inhibitor may be, e.g., a compound of Formula VI, VII, or VIII:

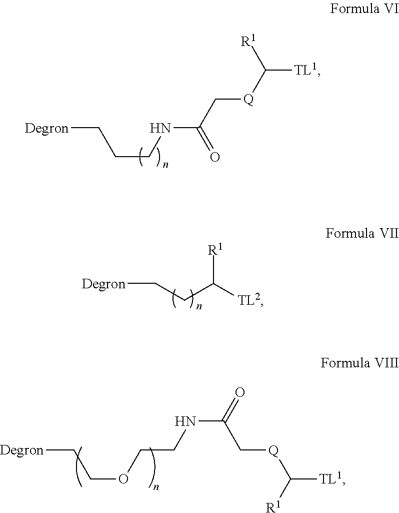

or a pharmaceutically acceptable salt thereof,
wherein
Degron is selected from the group consisting of:

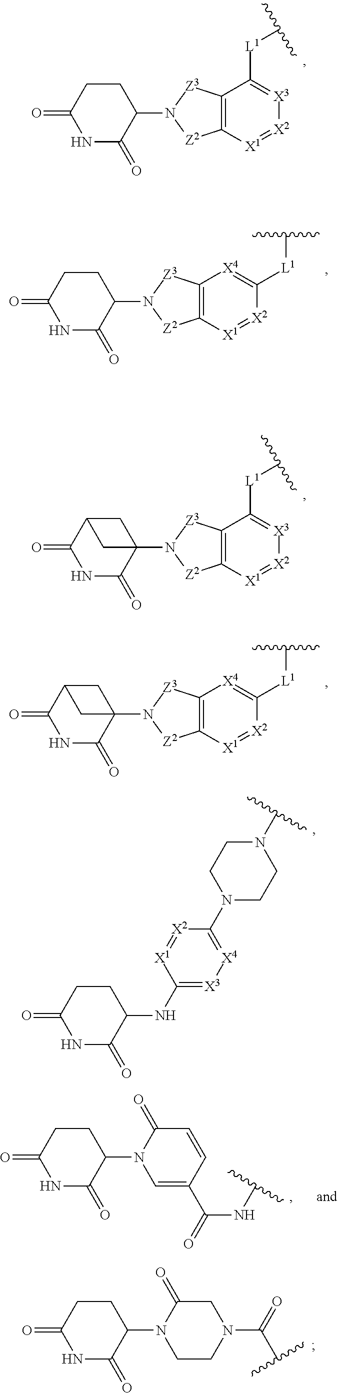

TL1 is a moiety that binds to BRD9 selected from the group consisting of:

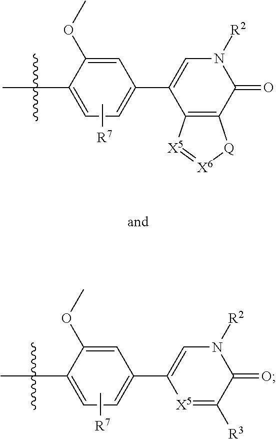

TL2 is a moiety that binds to BRD9 selected from the group consisting of:

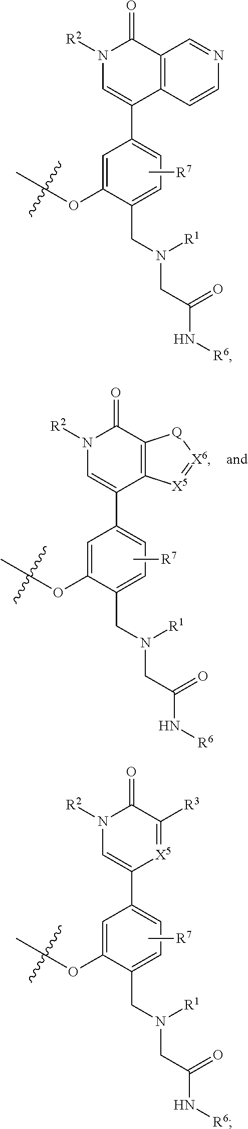

L¹ is selected from the group consisting of:

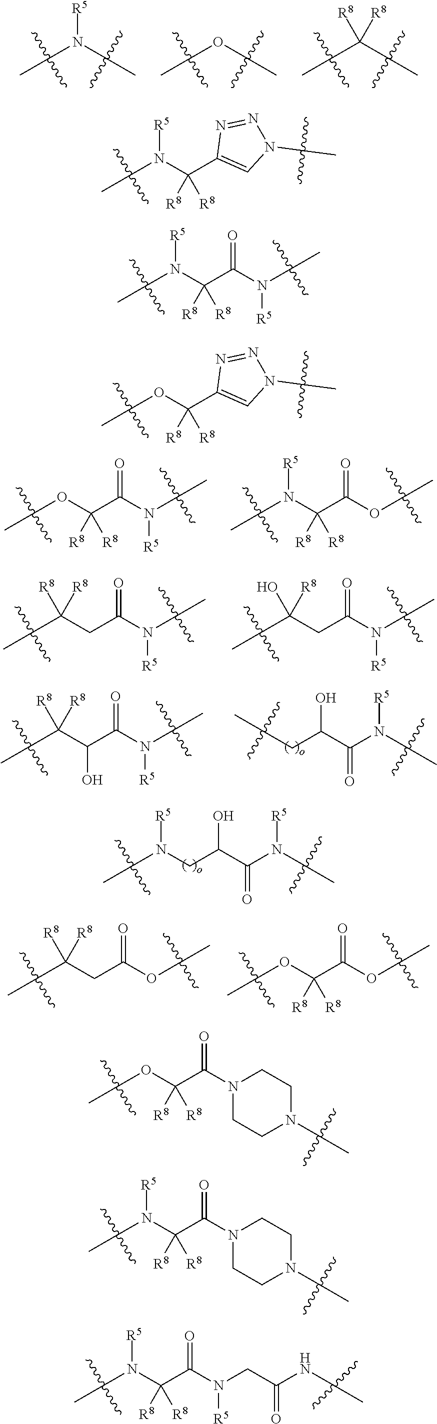

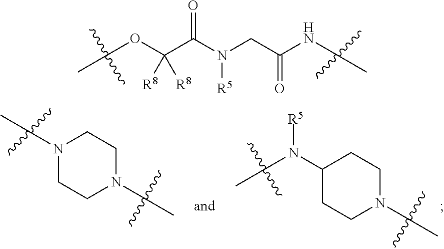

X¹, X², X³, and X⁴ are independently selected from CR⁴ and N, wherein no more than two of X¹, X², X³, and X⁴ may be selected to be N;
X⁵ and X⁶ are independently selected from CR⁴ and N;
Z² and Z³ are selected from —CH₂— and —C(O)— wherein at least one of Z² and Z³ is —C(O)—;
n is 0, 1, 2, 3, 4, 5, 6, 7, 8, 9, or 10;
is 1, 2, 3, or 4;
each Q is independently 0, S, or NR⁵;
R¹ is hydrogen or C₁-C₆ alkyl;
R², R³, and R⁵ are independently selected from hydrogen and C₁-C₆alkyl;
each R⁴ is independently selected from hydrogen, halogen, hydroxyl, C₁-C₆alkyl, C₁-C₆alkoxy, and C₁-C₆haloalkyl;
each R⁵ is independently hydrogen, C₁-C₆alkyl, or —C(O)alkyl;
R⁷ is selected from halogen, hydrogen, C₁-C₆alkyl, C₁-C₆alkoxy, and C₁-C₆haloalkyl; and
each R⁶ is independently selected from hydrogen, C₁-C₆alkyl, and C₁-C₆haloalkyl; or two R⁸ groups together with the carbon to which they are attached form a cyclopropyl group.

The BRD9 inhibitor may be, e.g., a compound of Formula IX:

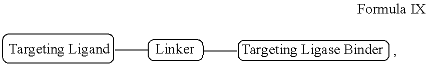

where
the Targeting Ligand is a group that is capable of binding to a bromodomain-containing protein, e.g., BRD9;
the Linker is a group that covalently links the Targeting Ligand to the Targeting Ligase Binder;
the Targeting Ligase Binder is a group that is capable of binding to a ligase (e.g., Cereblon E3 Ubiquitin ligase).

The Targeting Ligand may be, e.g., a group Formula TL-I or TL-II:

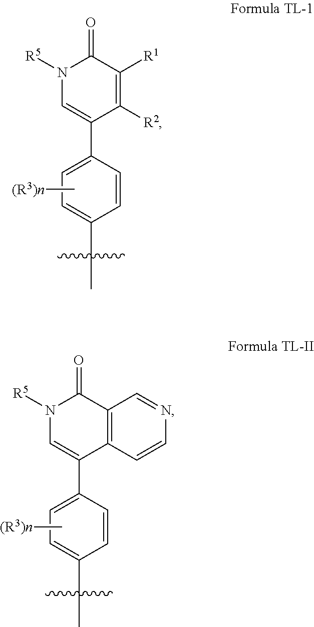

where
n is 0, 1, or 2;
R1 and R2 are independently selected from the group consisting of hydrogen and C1-6 alkyl; or R1 and R2 together with the atoms to which they are attached form an aryl or heteroaryl;
each R3 may be independently selected from the group consisting of C1-6 alkyl, C1-6 alkoxy, and halogen; and
R5 is selected from the group consisting of hydrogen and C1-6 alkyl.

The Linker may be, e.g., a group of Formula L-I:

-L1-X1-L2-X2-L3-, Formula L-1
where
L1 is selected from the group consisting of a bond, O, NR′, C(O), C1-6 alkylene,
C1-6 heteroalkylene, *C(O)—C1-6 alkylene, C(O)—C1-6 alkenylene*, C1-6 alkenylene, and *C(O)—C1-6 heteroalkylene, where * denotes the point of attachment of L1 to the Targeting Ligand;
X1 and X2 are each independently selected from the group consisting of a bond, carbocyclyl, and heterocyclyl, where the carbocyclyl and heterocyclyl are substituted with 0-4 occurrences of Ra, where each Ra is independently selected from the group consisting of C1-6 alkyl, C1-6 alkoxyl, and halogen;
L2 is selected from the group consisting of a bond, O, NR′, C1-6 alkylene, and C1-6 heteroalkylene; or X1-L2-X2 form a spiroheterocyclyl; and
L3 is selected from the group consisting of a bond, O, C(O), C1-6 alkylene, C1-6 heteroalkylene, *C(O)—C1-6 alkylene, *C(O)—C1-6 heteroalkylene, and *C(O)—C1-6 alkylene-O, where * denotes the point of attachment of L3 to X2; where no more than 2 of L1, X1, X2, L2, and L3 can simultaneously be a bond.

The Targeting Ligase Binder may be, e.g., a compound of Formula TLB-I:

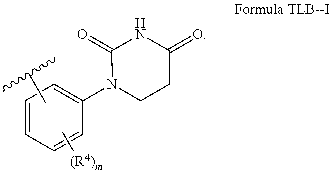

where
R4 is selected from the group consisting of C1-6 alkyl, C1-6 alkoxyl, and halogen; and m is 0, 1.

The Targeting Ligase Binder may be, e.g., a compound of Formula TLB-1:

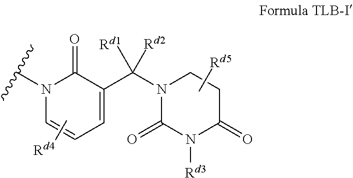

where
Rd1 and Rd2 are each independently selected from the group consisting of H, C1-6 alkyl, C1-6 alkoxyl, C1-6 haloalkyl, and C1-6 heteroalkyl;
Rd3 is H; Rd4 is selected from the group consisting of H, C1-6 alkyl, halo, C1-6 haloalkyl, and C1-6 heteroalkyl; and
Rd5 is selected from the group consisting of H, C1-6 alkyl, halo, C1-6 haloalkyl, and C1-6 heteroalkyl.

The BRD9 inhibitor may be, e.g., a compound of Formula IXa:

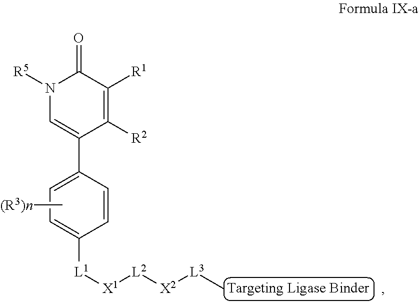

or a pharmaceutically acceptable salt thereof,
where
L1 is selected from the group consisting of a bond, O, NR′, C(O), C1-6 alkylene, C1-6 heteroalkylene, *C(O)—C1-6 alkylene, C(O)—C1-6 alkenylene*, C1-6 alkenylene, and *C(O)—C1-6 heteroalkylene, where * denotes the point of attachment of L1 to the Targeting Ligand;
X1 and X2 are each independently selected from the group consisting of a bond, carbocyclyl, and heterocyclyl, where the carbocyclyl and heterocyclyl are substituted with 0-4 occurrences of Ra, where each Ra is independently selected from the group consisting of C1-6 alkyl, C1-6 alkoxyl, and halogen;
L² is selected from the group consisting of a bond, O, NR′, C1-6 alkylene, and C1-6 heteroalkylene; or X1-L2-X2 form a spiroheterocyclyl; and
L3 is selected from the group consisting of a bond, O, C(O), C1-6 alkylene, C1-6 heteroalkylene, *C(O)—C1-6 alkylene, *C(O)—C1-6 heteroalkylene, and *C(O)—C1-6 alkylene-O, where * denotes the point of attachment of L3 to X2.

In some embodiments, no more than 2 of L1, X1, X2, L2, and L3 can simultaneously be a bond.

The BRD9 inhibitor may be, e.g., a compound of Formula X, XI, XII, XIII, XIV, or XV:

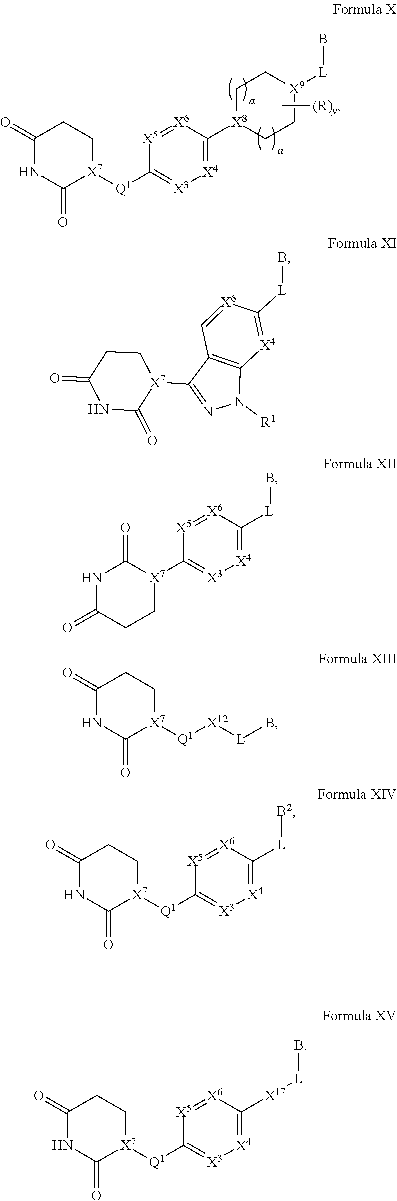

or a pharmaceutically acceptable salt thereof,
each a is independently 0, 1, or 2;
each y is independently 0, 1, or 2;
X3, X4, X5, and X6, are selected from the group consisting of N, CH and CR3, wherein no more than 3 of X3, X4, X5, and X6 are N;
X7 is N or CH;
X8 and X9 are each independently at each occurrence selected from the group consisting of N and CH; wherein at least one of X8 or X9 is CH;
X12 is a 5-membered heteroaryl group with 1, 2, or 3 atoms independently selected from N, O, and S, wherein X12 is optionally substituted with 1, 2, or 3 groups independently selected from R3;
X17 is aryl, heteroaryl, bicycle, or cycloalkyl, each of which is optionally substituted with 1, 2, 3, or 4 substituents independently selected from R3;
Q1 is independently at each occurrence selected from the group consisting of NH, N(alkyl), N(haloalkyl), CH₂, O, and S;
wherein if X7 is N, then Q1 is CH2; R is independently at each occurrence selected from the group consisting of hydrogen, C1-C4haloalkyl, C1-C4alkyl, fluorine, chlorine, bromine, iodine, CH2F, CHF2, CF3, CH2Cl, CHCl2, CCl3, CH2Br, CHBr2, and CBr3;
R1 is hydrogen, C1-4 alkyl, C1-4 haloalkyl, or cycloalkyl;
R3 is independently at each occurrence selected from the group consisting of hydrogen, hydroxyl, alkoxy, C1-4 alkyl, C1-4 haloalkyl, cycloalkyl, fluorine, chlorine, bromine, and iodine;
B is selected from B1 and B2;
B1 is selected from the group consisting of:

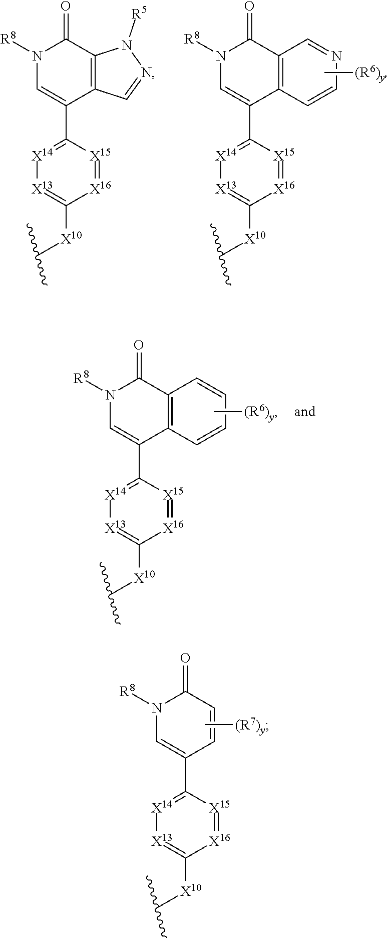

B2 is selected from the group consisting of:

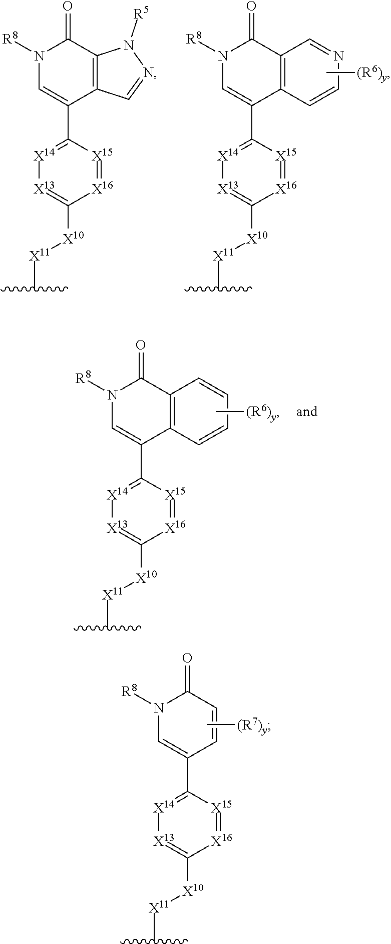

X10 is C(R7)₂, C(O), or O; X11 is heterocycle, heteroaryl, aryl, cycloalkyl, or a bicycle, each of which X11 groups is optionally substituted with 1, 2, 3, or 4 groups independently selected from R3; or X10 and X11 are taken together to form

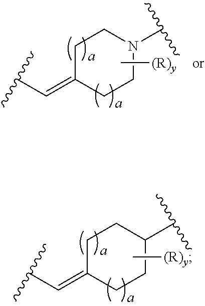

X13, X14, X15, and X16, are independently selected from the group consisting of N, CH and CR4, wherein no more than 3 of X13, X14, X15, and X16 are N;
each R4 is independently selected from hydrogen, aryl, heteroaryl, C1-4 alkoxy, C1-4 haloalkyl, C1-4 haloalkoxy, C1-4 alkyl, fluorine, chlorine, bromine, and iodine; wherein two R4 groups on adjacent carbon atoms may optionally combine to form a fused cycle, wherein the fused cycle is optionally substituted with 1, 2, or 3 R substituents, thus, non-limiting examples of

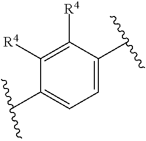

include

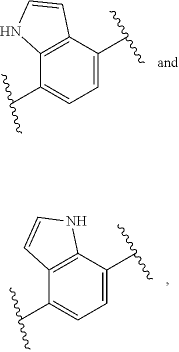 [SMILES: *c1ccc(*)c2[nH]ccc12 ; *c1ccc(*)c2[nH]ccc12]

wherein two R4 groups combined to form a pyrrole;
R5 is hydrogen, C1-C4alkyl, allyl, crotyl, alkenyl, alkynyl, haloalkyl, or cycloalkyl;
each R6 is independently selected from hydrogen, C1-4 alkoxy, C1-4 alkyl, C1-4 haloalkyl, fluorine, chlorine, bromine, and iodine;
each R7 is independently hydrogen or C1-4 alkyl;
R8 is hydrogen, C1-C4alkyl, allyl, crotyl, alkenyl, alkynyl, haloalkyl, or cycloalkyl; and
L is a bivalent linking group.

The BRD9 inhibitor may be, e.g., a compound selected from the group consisting of:

| Compound | |
|---|---|
| No. | Structure |
| F1 | 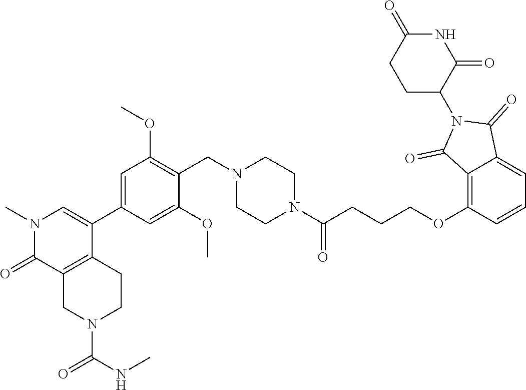 [SMILES: CNC(=O)N1CCc2c(-c3cc(OC)c(CN4CCN(C(=O)CCCOc5cccc6c5C(=O)N(C5CCC(=O)NC5=O)C6=O)CC4)c(OC)c3)cn(C)c(=O)c2C1] |
| F2 | 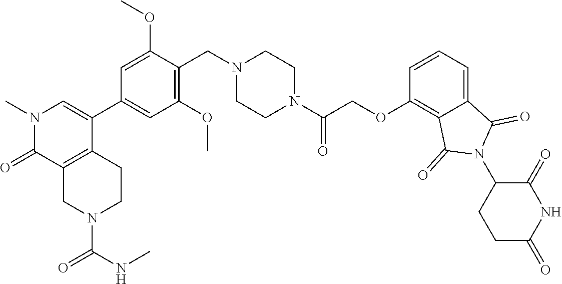 [SMILES: CNC(=O)N1CCc2c(-c3cc(OC)c(CN4CCN(C(=O)COc5cccc6c5C(=O)N(C5CCC(=O)NC5=O)C6=O)CC4)c(OC)c3)cn(C)c(=O)c2C1] |
| F3 | 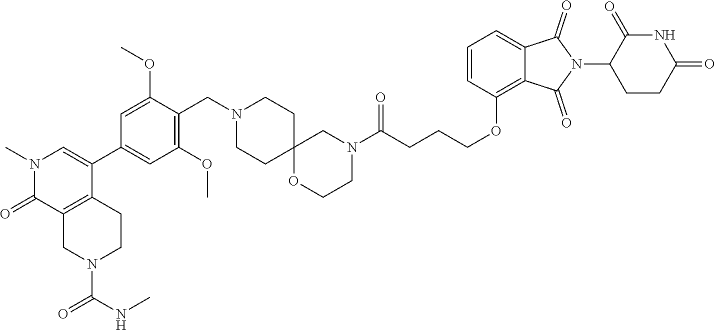 [SMILES: CNC(=O)N1CCc2c(-c3cc(OC)c(CN4CCC5(CC4)CN(C(=O)CCCOc4cccc6c4C(=O)N(C4CCC(=O)NC4=O)C6=O)CCO5)c(OC)c3)cn(C)c(=O)c2C1] |
| F4 | 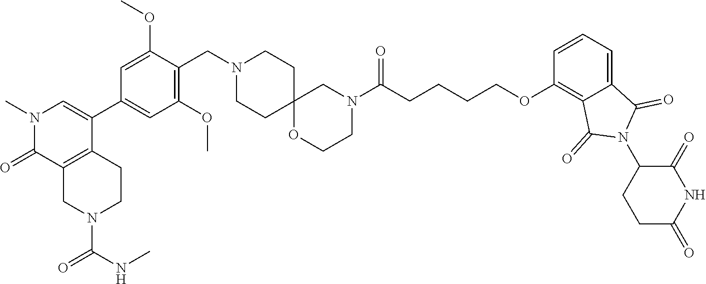 [SMILES: CNC(=O)N1CCc2c(-c3cc(OC)c(CN4CCC5(CC4)CN(C(=O)CCCCOc4cccc6c4C(=O)N(C4CCC(=O)NC4=O)C6=O)CCO5)c(OC)c3)cn(C)c(=O)c2C1] |
| F5 | 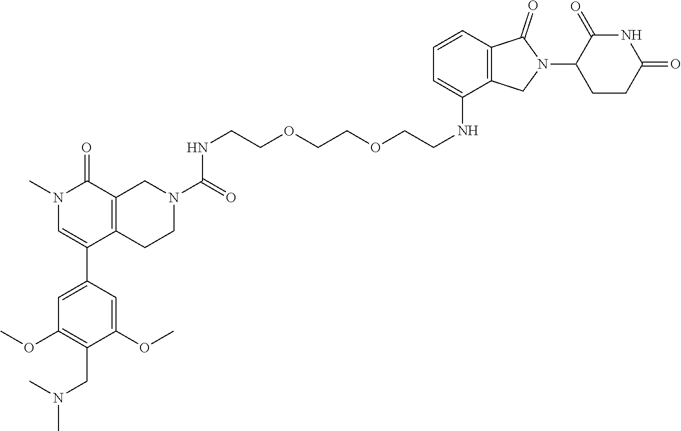 [SMILES: COc1cc(-c2cn(C)c(=O)c3c2CCN(C(=O)NCCOCCOCCNc2cccc4c2CN(C2CCC(=O)NC2=O)C4=O)C3)cc(OC)c1CN(C)C] |
| F6 | 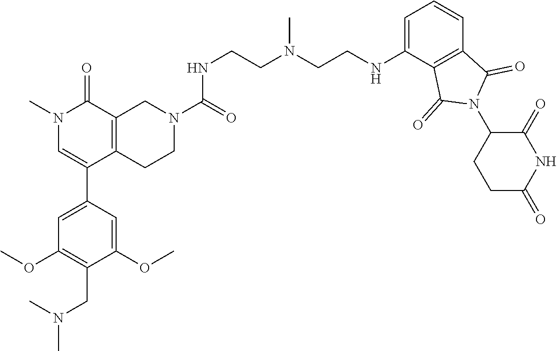 [SMILES: COc1cc(-c2cn(C)c(=O)c3c2CCN(C(=O)NCCN(C)CCNc2cccc4c2C(=O)N(C2CCC(=O)NC2=O)C4=O)C3)cc(OC)c1CN(C)C] |
| F7 | 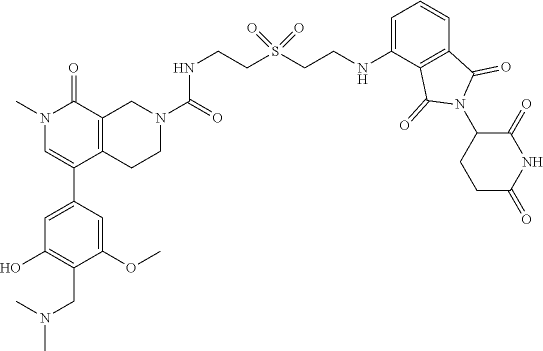 [SMILES: COc1cc(-c2cn(C)c(=O)c3c2CCN(C(=O)NCCS(=O)(=O)CCNc2cccc4c2C(=O)N(C2CCC(=O)NC2=O)C4=O)C3)cc(O)c1CN(C)C] |
| F8 | 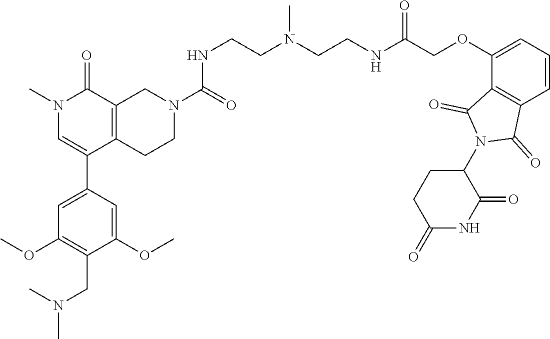 [SMILES: COc1cc(-c2cn(C)c(=O)c3c2CCN(C(=O)NCCN(C)CCNC(=O)COc2cccc4c2C(=O)N(C2CCC(=O)NC2=O)C4=O)C3)cc(OC)c1CN(C)C] |
| F9 | 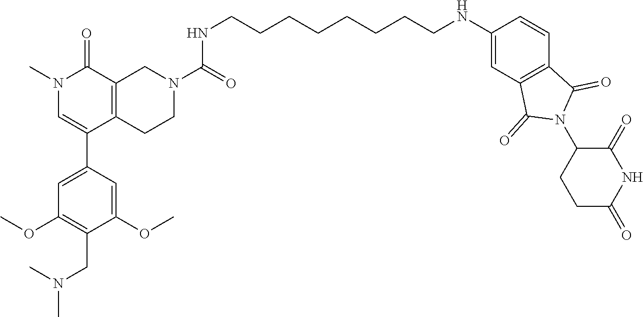 [SMILES: COc1cc(-c2cn(C)c(=O)c3c2CCN(C(=O)NCCCCCCCCNc2ccc4c(c2)C(=O)N(C2CCC(=O)NC2=O)C4=O)C3)cc(OC)c1CN(C)C] |
| F10 | 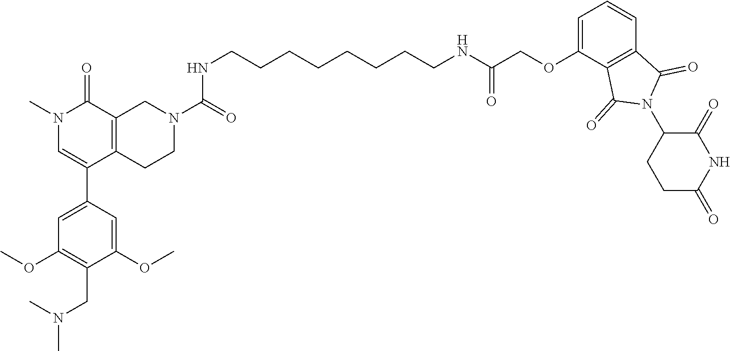 [SMILES: COc1cc(-c2cn(C)c(=O)c3c2CCN(C(=O)NCCCCCCCCNC(=O)COc2cccc4c2C(=O)N(C2CCC(=O)NC2=O)C4=O)C3)cc(OC)c1CN(C)C] |
| F11 | 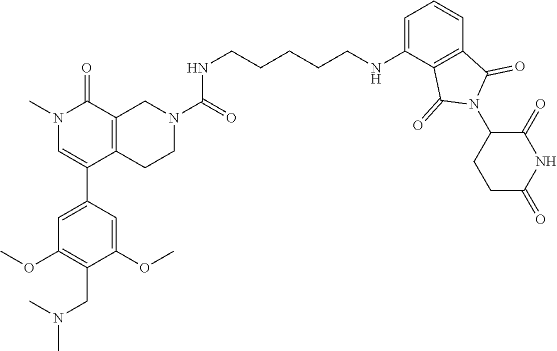 [SMILES: COc1cc(-c2cn(C)c(=O)c3c2CCN(C(=O)NCCCCCNc2cccc4c2C(=O)N(C2CCC(=O)NC2=O)C4=O)C3)cc(OC)c1CN(C)C] |
| F12 | 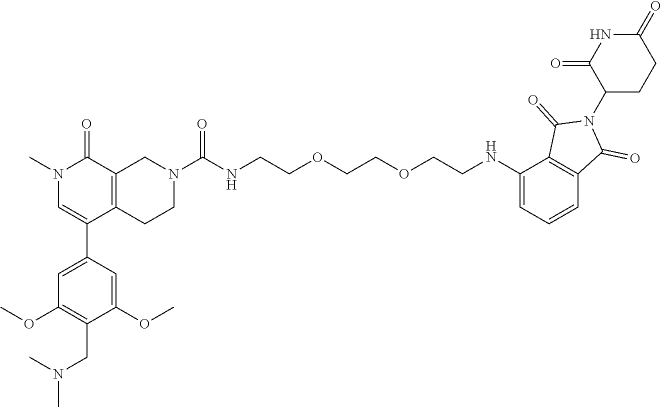 [SMILES: COc1cc(-c2cn(C)c(=O)c3c2CCN(C(=O)NCCOCCOCCNc2cccc4c2C(=O)N(C2CCC(=O)NC2=O)C4=O)C3)cc(OC)c1CN(C)C] |
| F13 | 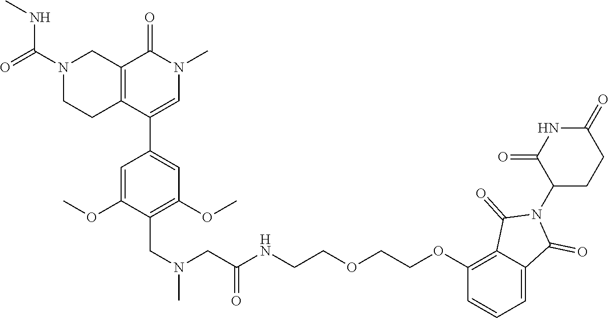 [SMILES: CNC(=O)N1CCc2c(-c3cc(OC)c(CN(C)CC(=O)NCCOCCOc4cccc5c4C(=O)N(C4CCC(=O)NC4=O)C5=O)c(OC)c3)cn(C)c(=O)c2C1] |
| F14 | 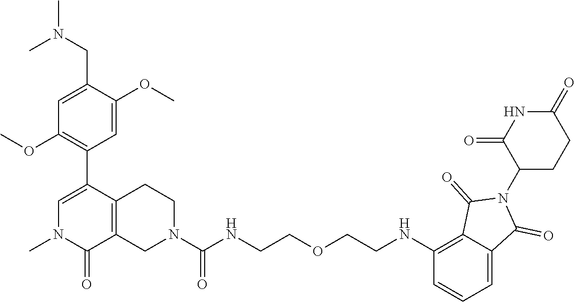 [SMILES: COc1cc(-c2cn(C)c(=O)c3c2CCN(C(=O)NCCOCCNc2cccc4c2C(=O)N(C2CCC(=O)NC2=O)C4=O)C3)c(OC)cc1CN(C)C] |
| F15 | 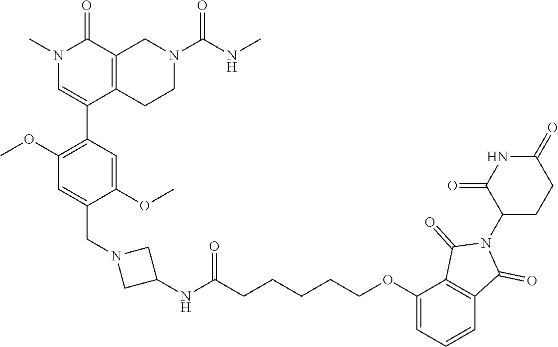 [SMILES: CNC(=O)N1CCc2c(-c3cc(OC)c(CN4CC(NC(=O)CCCCCOc5cccc6c5C(=O)N(C5CCC(=O)NC5=O)C6=O)C4)cc3OC)cn(C)c(=O)c2C1] |
| F16 | 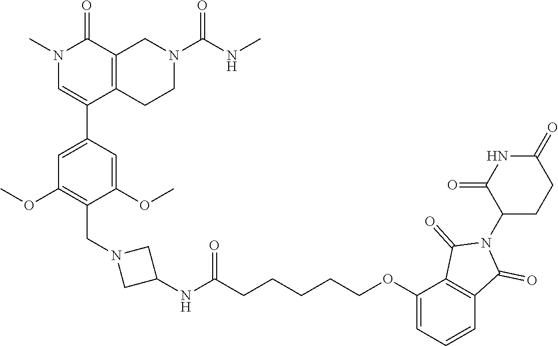 [SMILES: CNC(=O)N1CCc2c(-c3cc(OC)c(CN4CC(NC(=O)CCCCCOc5cccc6c5C(=O)N(C5CCC(=O)NC5=O)C6=O)C4)c(OC)c3)cn(C)c(=O)c2C1] |
| F17 | 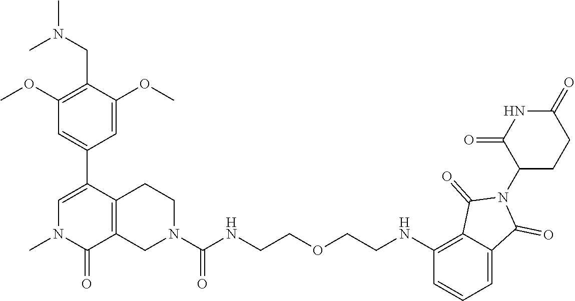 [SMILES: COc1cc(-c2cn(C)c(=O)c3c2CCN(C(=O)NCCOCCNc2cccc4c2C(=O)N(C2CCC(=O)NC2=O)C4=O)C3)cc(OC)c1CN(C)C] |
| F18 | 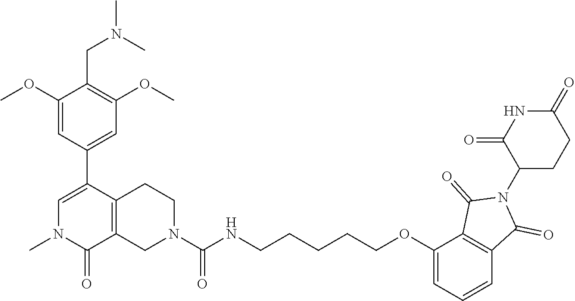 [SMILES: COc1cc(-c2cn(C)c(=O)c3c2CCN(C(=O)NCCCCCOc2cccc4c2C(=O)N(C2CCC(=O)NC2=O)C4=O)C3)cc(OC)c1CN(C)C] |
| F19 | 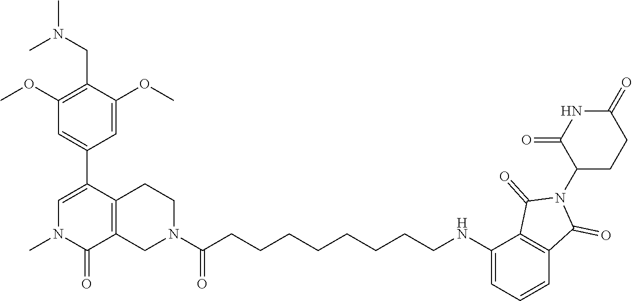 [SMILES: COc1cc(-c2cn(C)c(=O)c3c2CCN(C(=O)CCCCCCCCNc2cccc4c2C(=O)N(C2CCC(=O)NC2=O)C4=O)C3)cc(OC)c1CN(C)C] |
| F20 | 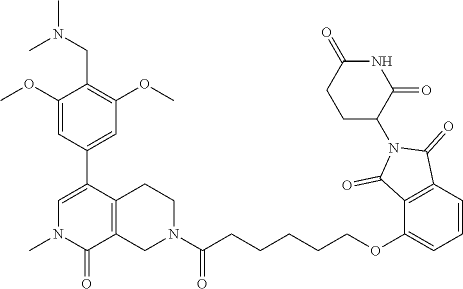 [SMILES: COc1cc(-c2cn(C)c(=O)c3c2CCN(C(=O)CCCCCOc2cccc4c2C(=O)N(C2CCC(=O)NC2=O)C4=O)C3)cc(OC)c1CN(C)C] |
| F21 | 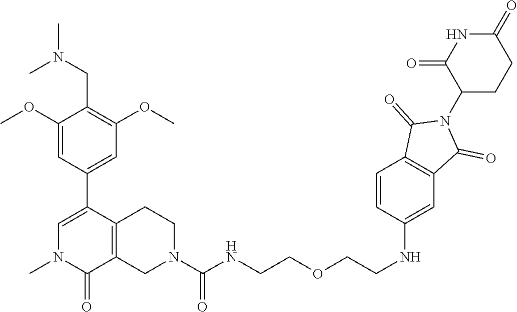 [SMILES: COc1cc(-c2cn(C)c(=O)c3c2CCN(C(=O)NCCOCCNc2ccc4c(c2)C(=O)N(C2CCC(=O)NC2=O)C4=O)C3)cc(OC)c1CN(C)C] |
| F22 | 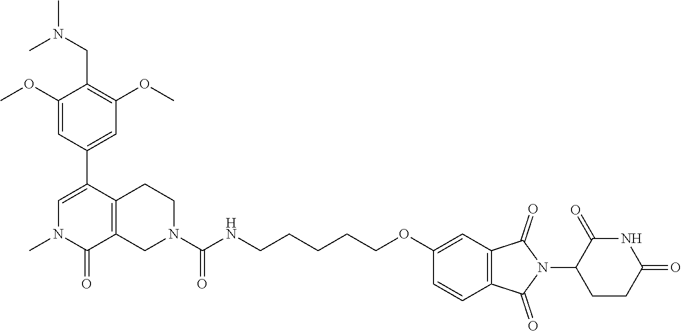 [SMILES: COc1cc(-c2cn(C)c(=O)c3c2CCN(C(=O)NCCCCCOc2ccc4c(c2)C(=O)N(C2CCC(=O)NC2=O)C4=O)C3)cc(OC)c1CN(C)C] |
| F23 | 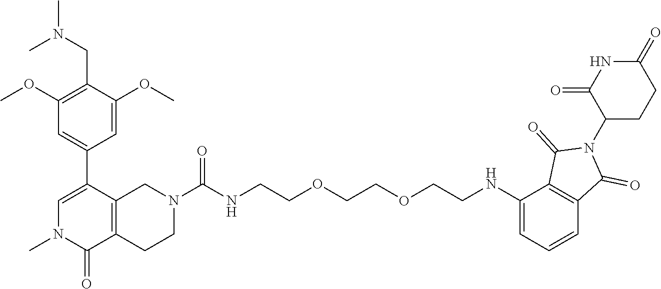 [SMILES: COc1cc(-c2cn(C)c(=O)c3c2CN(C(=O)NCCOCCOCCNc2cccc4c2C(=O)N(C2CCC(=O)NC2=O)C4=O)CC3)cc(OC)c1CN(C)C] |
| F24 | 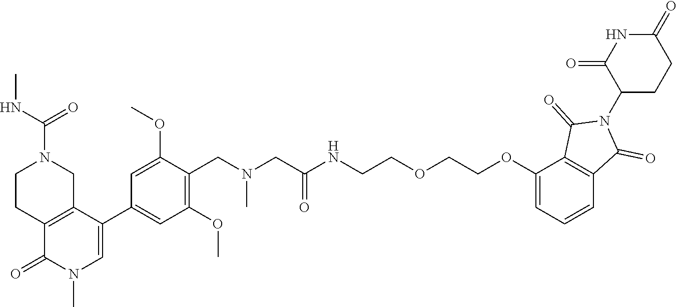 [SMILES: CNC(=O)N1CCc2c(c(-c3cc(OC)c(CN(C)CC(=O)NCCOCCOc4cccc5c4C(=O)N(C4CCC(=O)NC4=O)C5=O)c(OC)c3)cn(C)c2=O)C1] |
| F25 | 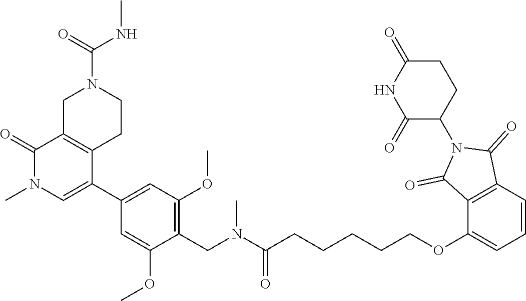 [SMILES: CNC(=O)N1CCc2c(-c3cc(OC)c(CN(C)C(=O)CCCCCOc4cccc5c4C(=O)N(C4CCC(=O)NC4=O)C5=O)c(OC)c3)cn(C)c(=O)c2C1] |
| F26 | 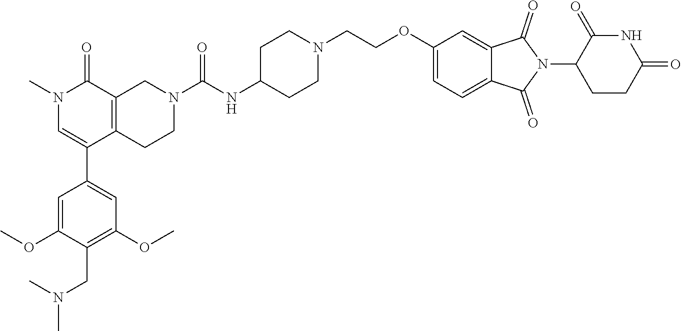 [SMILES: COc1cc(-c2cn(C)c(=O)c3c2CCN(C(=O)NC2CCN(CCOc4ccc5c(c4)C(=O)N(C4CCC(=O)NC4=O)C5=O)CC2)C3)cc(OC)c1CN(C)C] |
| F27 | 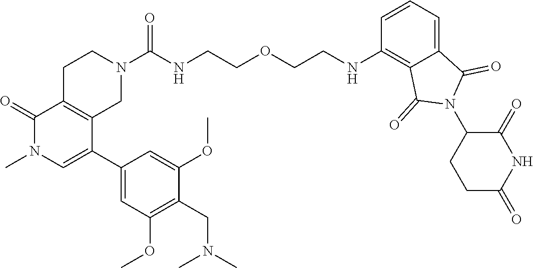 [SMILES: COc1cc(-c2cn(C)c(=O)c3c2CN(C(=O)NCCOCCNc2cccc4c2C(=O)N(C2CCC(=O)NC2=O)C4=O)CC3)cc(OC)c1CN(C)C] |
| F28 | 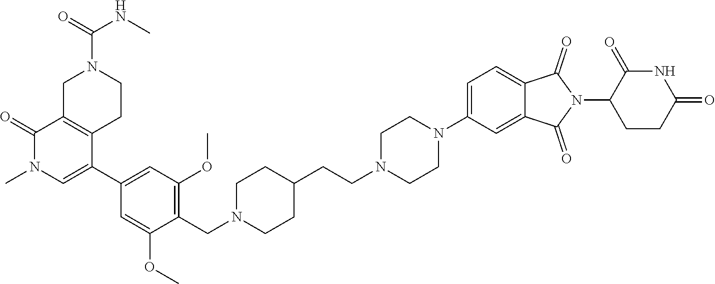 [SMILES: CNC(=O)N1CCc2c(-c3cc(OC)c(CN4CCC(CCN5CCN(c6ccc7c(c6)C(=O)N(C6CCC(=O)NC6=O)C7=O)CC5)CC4)c(OC)c3)cn(C)c(=O)c2C1] |
| F29 | 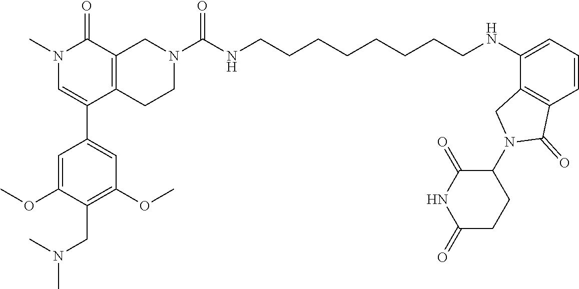 [SMILES: COc1cc(-c2cn(C)c(=O)c3c2CCN(C(=O)NCCCCCCCCNc2cccc4c2CN(C2CCC(=O)NC2=O)C4=O)C3)cc(OC)c1CN(C)C] |
| F30 | 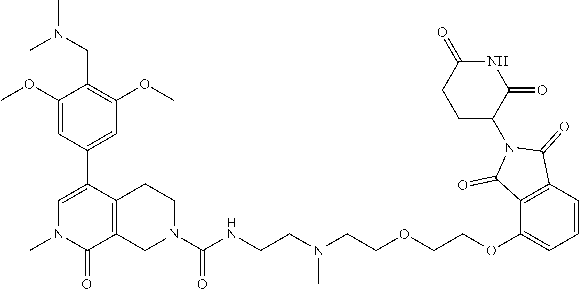 [SMILES: COc1cc(-c2cn(C)c(=O)c3c2CCN(C(=O)NCCN(C)CCOCCOc2cccc4c2C(=O)N(C2CCC(=O)NC2=O)C4=O)C3)cc(OC)c1CN(C)C] |
| F31 | 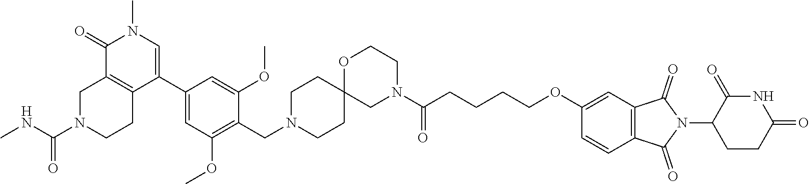 [SMILES: CNC(=O)N1CCc2c(-c3cc(OC)c(CN4CCC5(CC4)CN(C(=O)CCCCOc4ccc6c(c4)C(=O)N(C4CCC(=O)NC4=O)C6=O)CCO5)c(OC)c3)cn(C)c(=O)c2C1] |
| F32 | 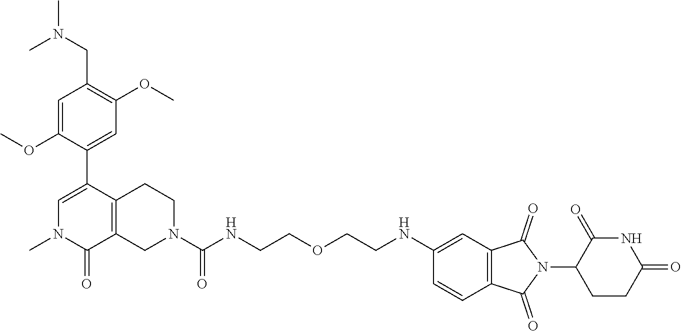 [SMILES: COc1cc(-c2cn(C)c(=O)c3c2CCN(C(=O)NCCOCCNc2ccc4c(c2)C(=O)N(C2CCC(=O)NC2=O)C4=O)C3)c(OC)cc1CN(C)C] |
| F33 | 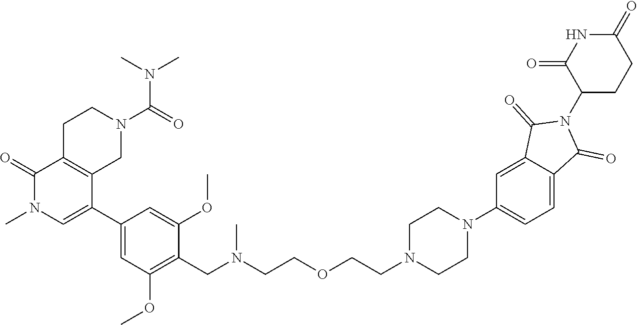 [SMILES: COc1cc(-c2cn(C)c(=O)c3c2CN(C(=O)N(C)C)CC3)cc(OC)c1CN(C)CCOCCN1CCN(c2ccc3c(c2)C(=O)N(C2CCC(=O)NC2=O)C3=O)CC1] |
| F34 | 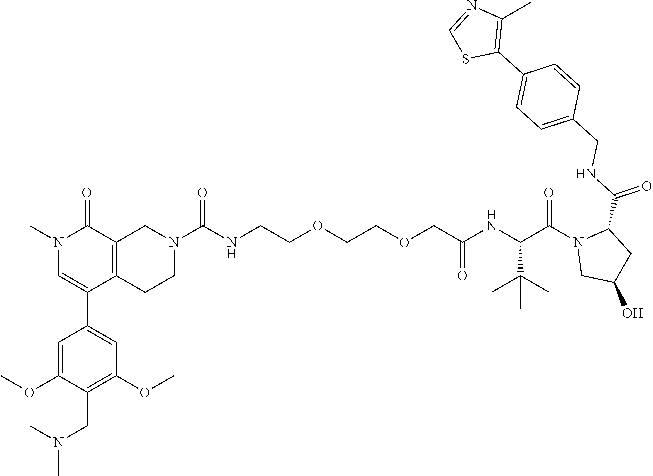 [SMILES: COc1cc(-c2cn(C)c(=O)c3c2CCN(C(=O)NCCOCCOCC(=O)N[C@H](C(=O)N2C[C@H](O)C[C@H]2C(=O)NCc2ccc(-c4scnc4C)cc2)C(C)(C)C)C3)cc(OC)c1CN(C)C] |
| F35 | 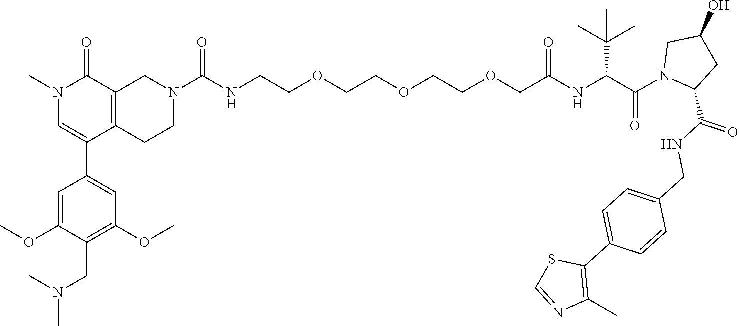 [SMILES: COc1cc(-c2cn(C)c(=O)c3c2CCN(C(=O)NCCOCCOCCOCC(=O)N[C@@H](C(=O)N2C[C@@H](O)C[C@@H]2C(=O)NCc2ccc(-c4scnc4C)cc2)C(C)(C)C)C3)cc(OC)c1CN(C)C] |
| F36 | 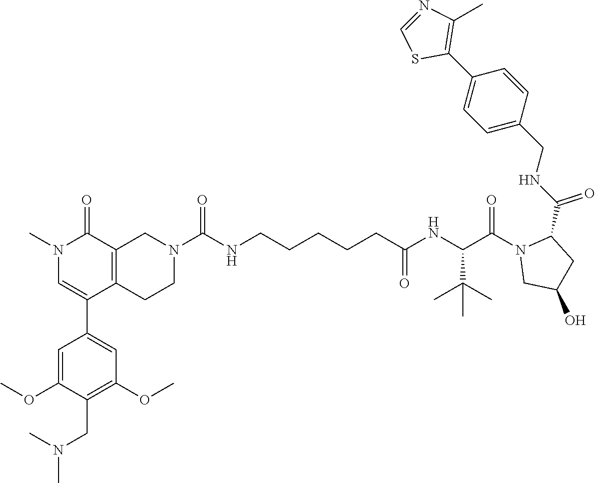 [SMILES: COc1cc(-c2cn(C)c(=O)c3c2CCN(C(=O)NCCCCCC(=O)N[C@H](C(=O)N2C[C@H](O)C[C@H]2C(=O)NCc2ccc(-c4scnc4C)cc2)C(C)(C)C)C3)cc(OC)c1CN(C)C] |
| F37 | 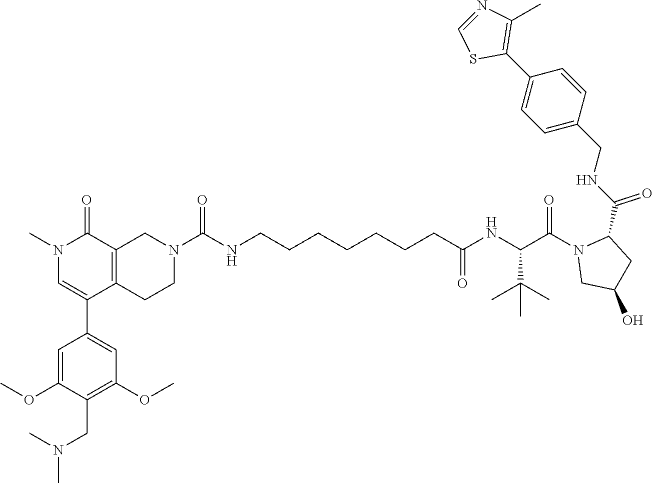 [SMILES: COc1cc(-c2cn(C)c(=O)c3c2CCN(C(=O)NCCCCCCCC(=O)N[C@H](C(=O)N2C[C@H](O)C[C@H]2C(=O)NCc2ccc(-c4scnc4C)cc2)C(C)(C)C)C3)cc(OC)c1CN(C)C] |
| F38 | 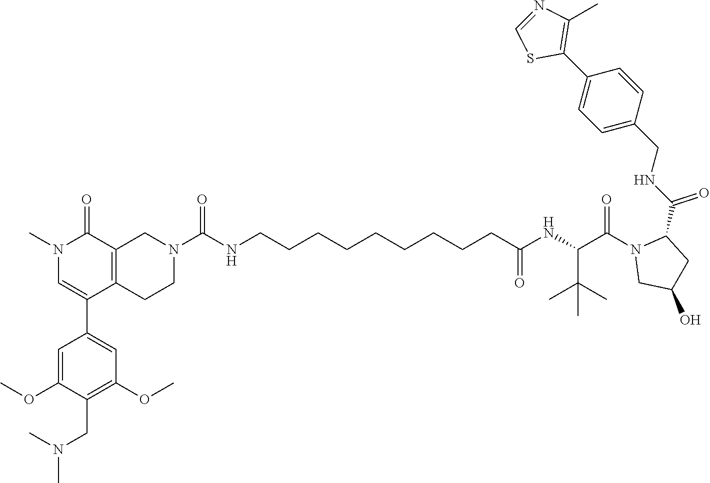 [SMILES: COc1cc(-c2cn(C)c(=O)c3c2CCN(C(=O)NCCCCCCCCCC(=O)N[C@H](C(=O)N2C[C@H](O)C[C@H]2C(=O)NCc2ccc(-c4scnc4C)cc2)C(C)(C)C)C3)cc(OC)c1CN(C)C] |
| F39 | 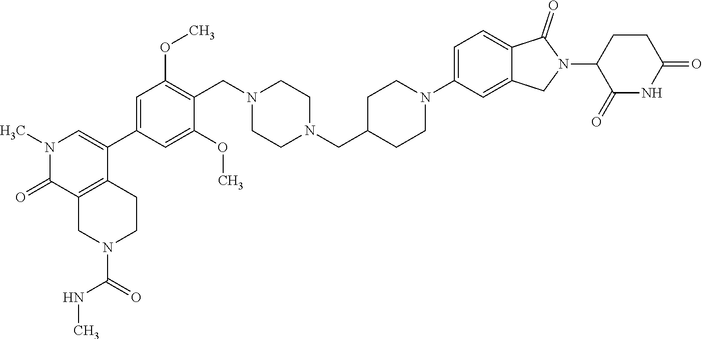 [SMILES: CNC(=O)N1CCc2c(-c3cc(OC)c(CN4CCN(CC5CCN(c6ccc7c(c6)CN(C6CCC(=O)NC6=O)C7=O)CC5)CC4)c(OC)c3)cn(C)c(=O)c2C1] |
| F40 | 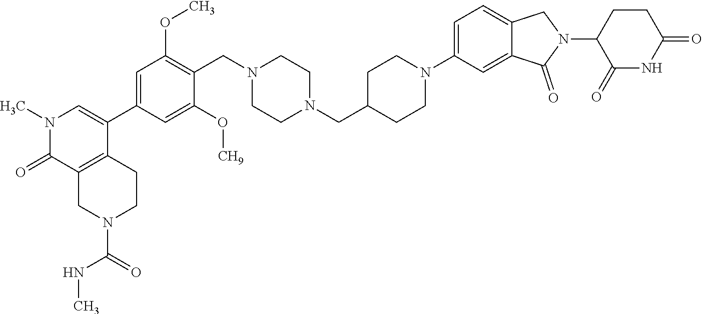 |
| F41 | 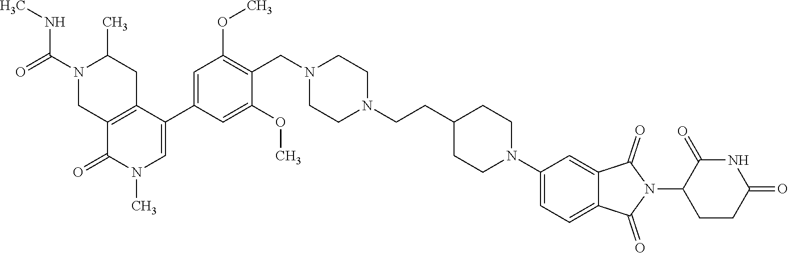 [SMILES: CNC(=O)N1Cc2c(c(-c3cc(OC)c(CN4CCN(CCC5CCN(c6ccc7c(c6)C(=O)N(C6CCC(=O)NC6=O)C7=O)CC5)CC4)c(OC)c3)cn(C)c2=O)CC1C] |
| F42 | 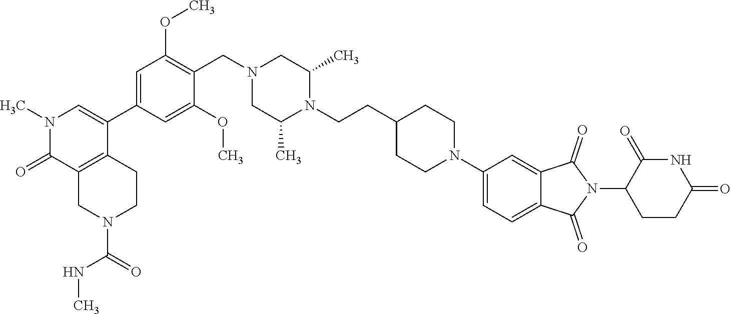 [SMILES: CNC(=O)N1CCc2c(-c3cc(OC)c(CN4C[C@@H](C)N(CCC5CCN(c6ccc7c(c6)C(=O)N(C6CCC(=O)NC6=O)C7=O)CC5)[C@@H](C)C4)c(OC)c3)cn(C)c(=O)c2C1] |
| F43 | 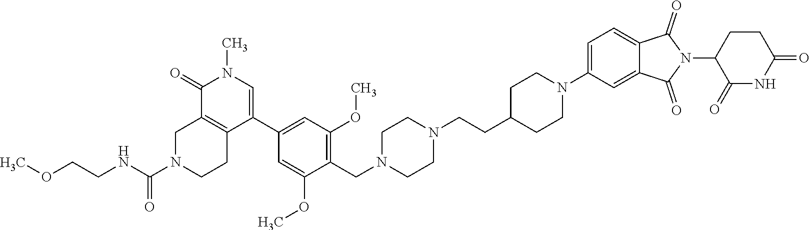 [SMILES: COCCNC(=O)N1CCc2c(-c3cc(OC)c(CN4CCN(CCC5CCN(c6ccc7c(c6)C(=O)N(C6CCC(=O)NC6=O)C7=O)CC5)CC4)c(OC)c3)cn(C)c(=O)c2C1] |
| F44 | 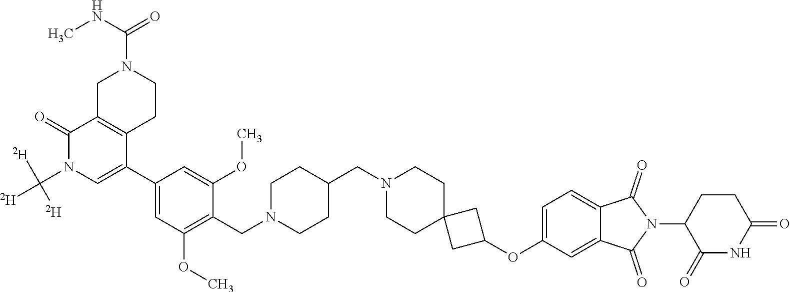 [SMILES: [2H]C([2H])([2H])n1cc(-c2cc(OC)c(CN3CCC(CN4CCC5(CC4)CC(Oc4ccc6c(c4)C(=O)N(C4CCC(=O)NC4=O)C6=O)C5)CC3)c(OC)c2)c2c(c1=O)CN(C(=O)NC)CC2] |
| F45 | 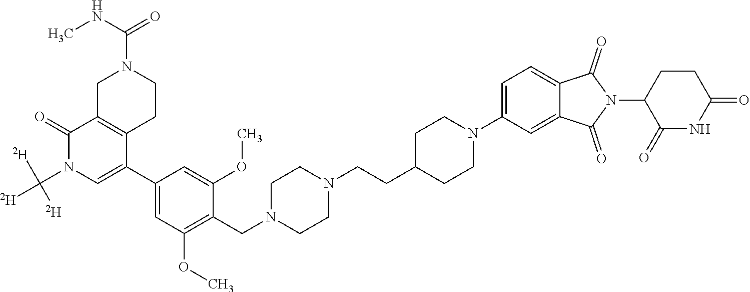 [SMILES: [2H]C([2H])([2H])n1cc(-c2cc(OC)c(CN3CCN(CCC4CCN(c5ccc6c(c5)C(=O)N(C5CCC(=O)NC5=O)C6=O)CC4)CC3)c(OC)c2)c2c(c1=O)CN(C(=O)NC)CC2] |
| F46 | 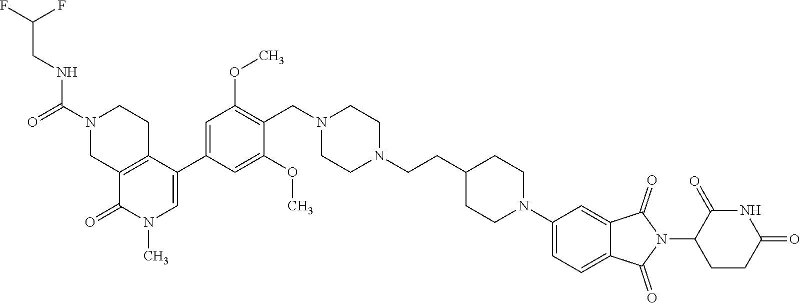 [SMILES: COc1cc(-c2cn(C)c(=O)c3c2CCN(C(=O)NCC(F)F)C3)cc(OC)c1CN1CCN(CCC2CCN(c3ccc4c(c3)C(=O)N(C3CCC(=O)NC3=O)C4=O)CC2)CC1] |
| F47 | 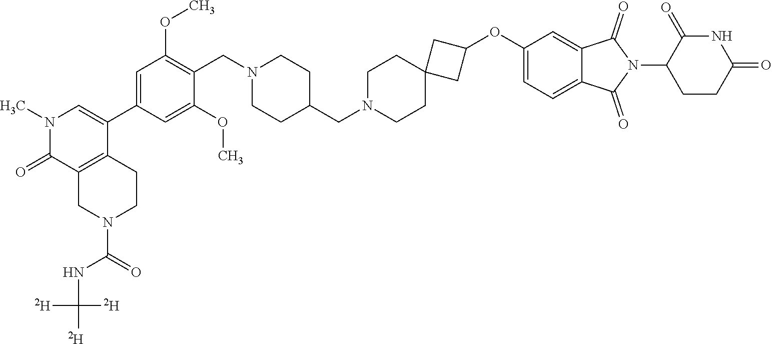 [SMILES: [2H]C([2H])([2H])NC(=O)N1CCc2c(-c3cc(OC)c(CN4CCC(CN5CCC6(CC5)CC(Oc5ccc7c(c5)C(=O)N(C5CCC(=O)NC5=O)C7=O)C6)CC4)c(OC)c3)cn(C)c(=O)c2C1] |
| F48 | 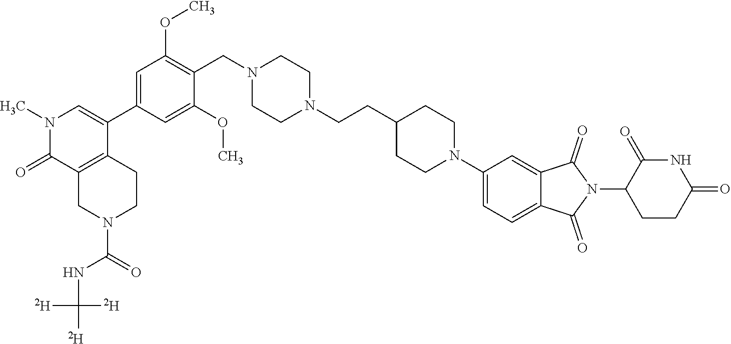 [SMILES: [2H]C([2H])([2H])NC(=O)N1CCc2c(-c3cc(OC)c(CN4CCN(CCC5CCN(c6ccc7c(c6)C(=O)N(C6CCC(=O)NC6=O)C7=O)CC5)CC4)c(OC)c3)cn(C)c(=O)c2C1] |
| F49 | 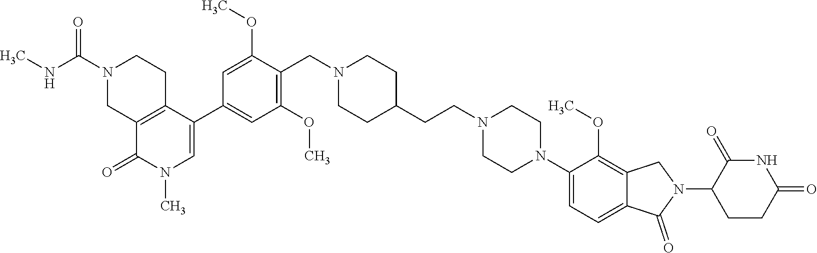 [SMILES: CNC(=O)N1CCc2c(-c3cc(OC)c(CN4CCC(CCN5CCN(c6ccc7c(c6OC)CN(C6CCC(=O)NC6=O)C7=O)CC5)CC4)c(OC)c3)cn(C)c(=O)c2C1] |
| F50 | 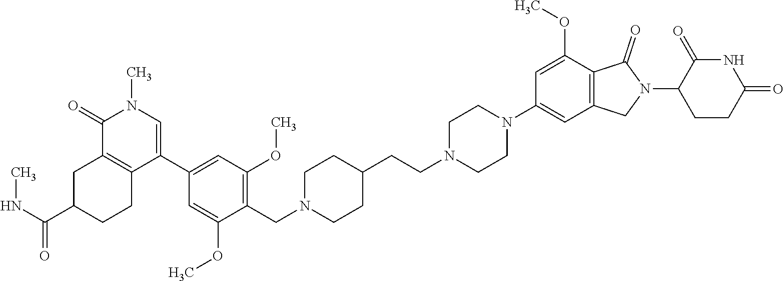 [SMILES: CNC(=O)C1CCc2c(-c3cc(OC)c(CN4CCC(CCN5CCN(c6cc7c(c(OC)c6)C(=O)N(C6CCC(=O)NC6=O)C7)CC5)CC4)c(OC)c3)cn(C)c(=O)c2C1] |
| F51 | 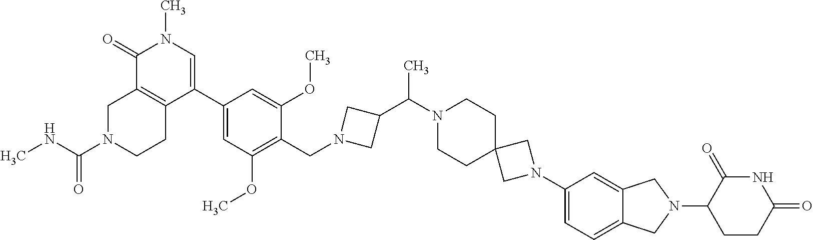 [SMILES: CNC(=O)N1CCc2c(-c3cc(OC)c(CN4CC(C(C)N5CCC6(CC5)CN(c5ccc7c(c5)CN(C5CCC(=O)NC5=O)C7)C6)C4)c(OC)c3)cn(C)c(=O)c2C1] |
| F52 | 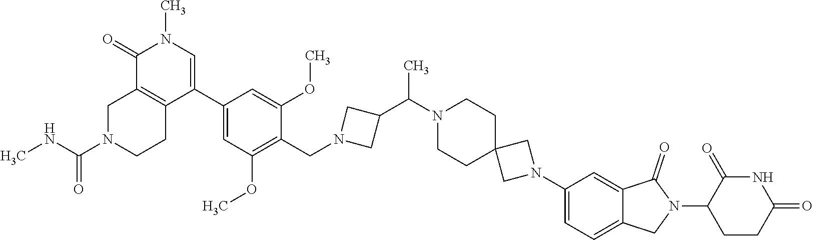 [SMILES: CNC(=O)N1CCc2c(-c3cc(OC)c(CN4CC(C(C)N5CCC6(CC5)CN(c5ccc7c(c5)C(=O)N(C5CCC(=O)NC5=O)C7)C6)C4)c(OC)c3)cn(C)c(=O)c2C1] |
| F53 | 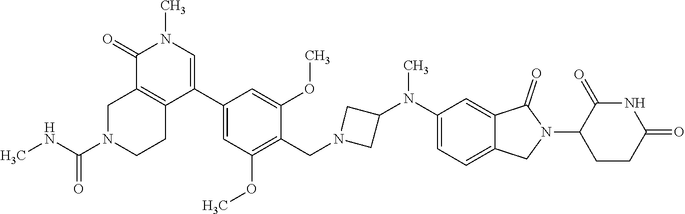 [SMILES: CNC(=O)N1CCc2c(-c3cc(OC)c(CN4CC(N(C)c5ccc6c(c5)C(=O)N(C5CCC(=O)NC5=O)C6)C4)c(OC)c3)cn(C)c(=O)c2C1] |
| F54 | 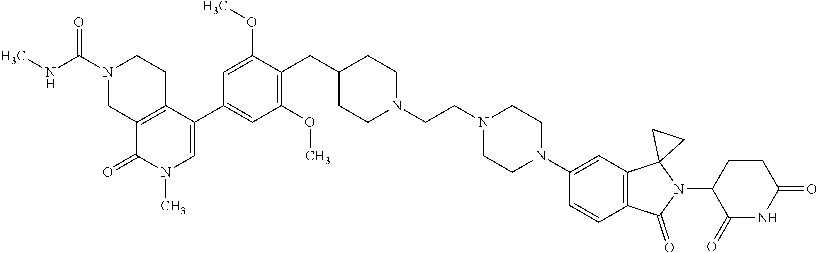 [SMILES: CNC(=O)N1CCc2c(-c3cc(OC)c(CC4CCN(CCN5CCN(c6ccc7c(c6)C6(CC6)N(C6CCC(=O)NC6=O)C7=O)CC5)CC4)c(OC)c3)cn(C)c(=O)c2C1] |
| F55 | 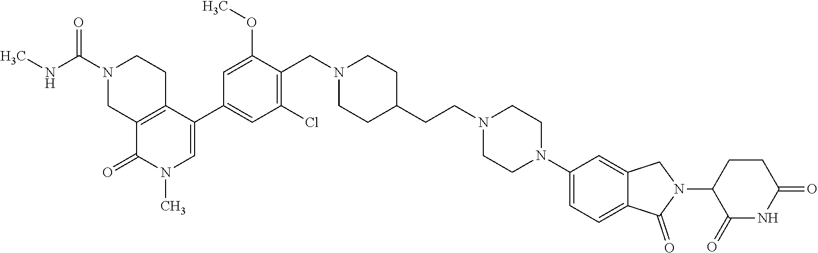 [SMILES: CNC(=O)N1CCc2c(-c3cc(Cl)c(CN4CCC(CCN5CCN(c6ccc7c(c6)CN(C6CCC(=O)NC6=O)C7=O)CC5)CC4)c(OC)c3)cn(C)c(=O)c2C1] |
| F56 | 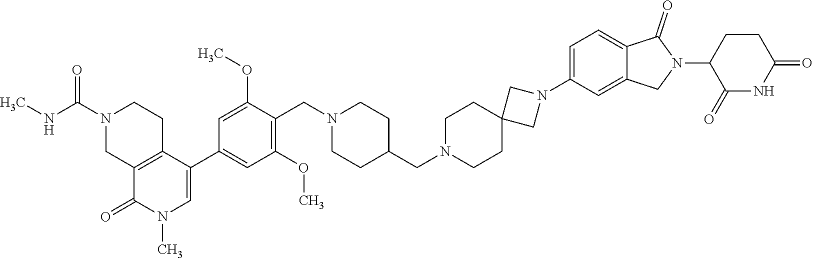 [SMILES: CNC(=O)N1CCc2c(-c3cc(OC)c(CN4CCC(CN5CCC6(CC5)CN(c5ccc7c(c5)CN(C5CCC(=O)NC5=O)C7=O)C6)CC4)c(OC)c3)cn(C)c(=O)c2C1] |
| F57 | 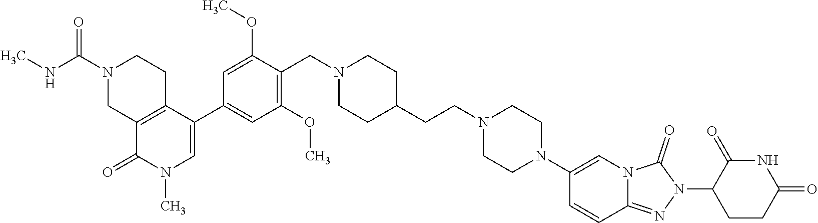 [SMILES: CNC(=O)N1CCc2c(-c3cc(OC)c(CN4CCC(CCN5CCN(c6ccc7nn(C8CCC(=O)NC8=O)c(=O)n7c6)CC5)CC4)c(OC)c3)cn(C)c(=O)c2C1] |
| F58 | 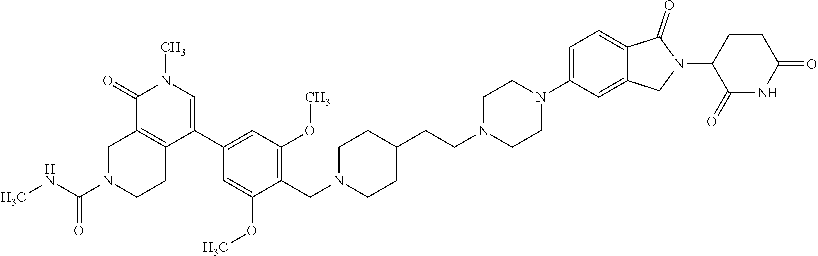 [SMILES: CNC(=O)N1CCc2c(-c3cc(OC)c(CN4CCC(CCN5CCN(c6ccc7c(c6)CN(C6CCC(=O)NC6=O)C7=O)CC5)CC4)c(OC)c3)cn(C)c(=O)c2C1] |
| F59 | 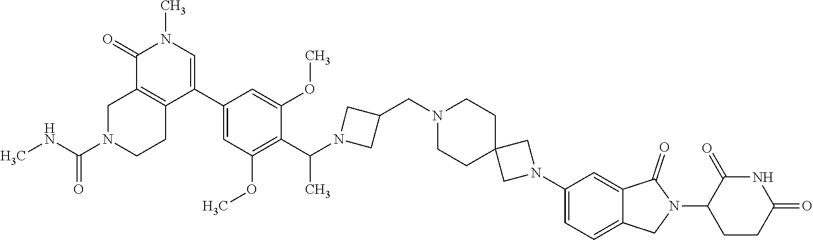 [SMILES: CNC(=O)N1CCc2c(-c3cc(OC)c(C(C)N4CC(CN5CCC6(CC5)CN(c5ccc7c(c5)C(=O)N(C5CCC(=O)NC5=O)C7)C6)C4)c(OC)c3)cn(C)c(=O)c2C1] |
| F60 | 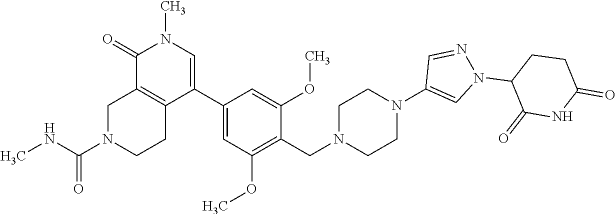 [SMILES: CNC(=O)N1CCc2c(-c3cc(OC)c(CN4CCN(c5cnn(C6CCC(=O)NC6=O)c5)CC4)c(OC)c3)cn(C)c(=O)c2C1] |
| F61 | 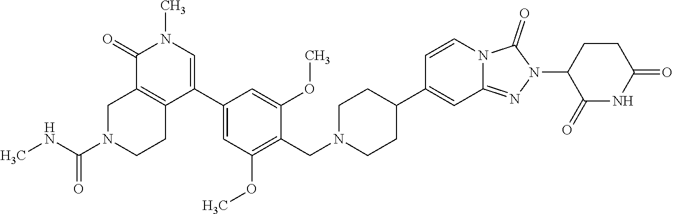 [SMILES: CNC(=O)N1CCc2c(-c3cc(OC)c(CN4CCC(c5ccn6c(=O)n(C7CCC(=O)NC7=O)nc6c5)CC4)c(OC)c3)cn(C)c(=O)c2C1] |
| F62 | 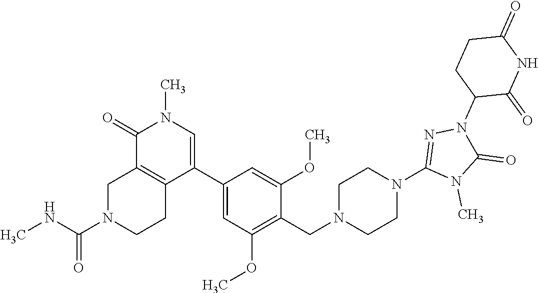 [SMILES: CNC(=O)N1CCc2c(-c3cc(OC)c(CN4CCN(c5nn(C6CCC(=O)NC6=O)c(=O)n5C)CC4)c(OC)c3)cn(C)c(=O)c2C1] |
| F63 | 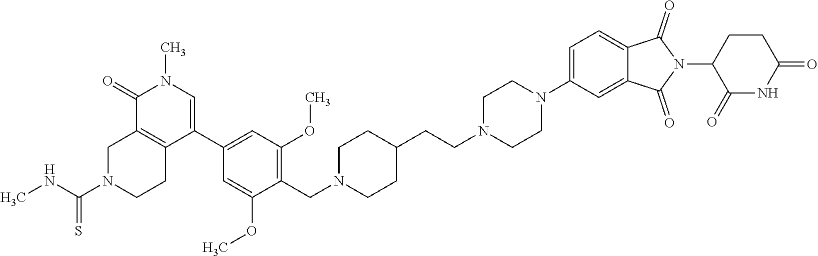 [SMILES: CNC(=S)N1CCc2c(-c3cc(OC)c(CN4CCC(CCN5CCN(c6ccc7c(c6)C(=O)N(C6CCC(=O)NC6=O)C7=O)CC5)CC4)c(OC)c3)cn(C)c(=O)c2C1] |
| F64 | 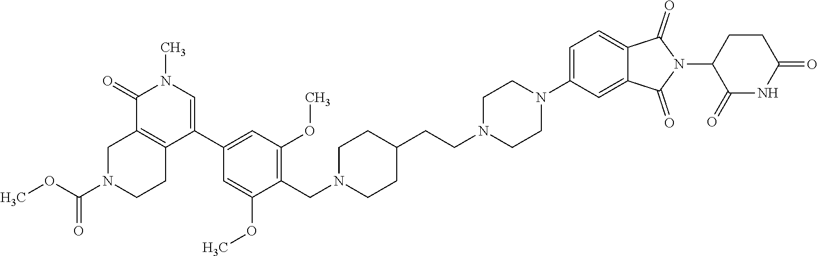 [SMILES: COC(=O)N1CCc2c(-c3cc(OC)c(CN4CCC(CCN5CCN(c6ccc7c(c6)C(=O)N(C6CCC(=O)NC6=O)C7=O)CC5)CC4)c(OC)c3)cn(C)c(=O)c2C1] |
| F65 | 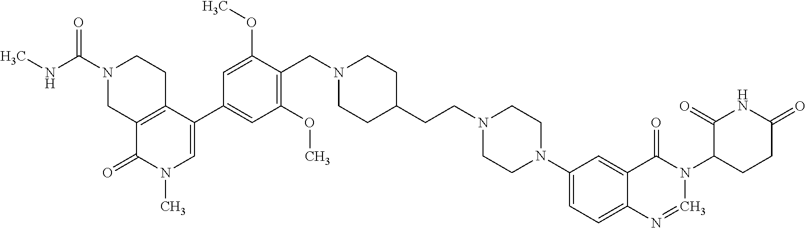 [SMILES: CNC(=O)N1CCc2c(-c3cc(OC)c(CN4CCC(CCN5CCN(c6ccc7ncn(C8CCC(=O)NC8=O)c(=O)c7c6)CC5)CC4)c(OC)c3)cn(C)c(=O)c2C1] |
| F66 | 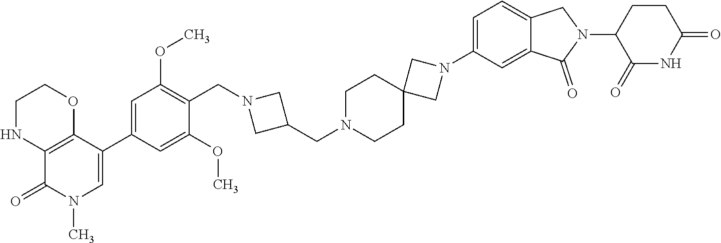 [SMILES: COc1cc(-c2cn(C)c(=O)c3c2OCCN3)cc(OC)c1CN1CC(CN2CCC3(CC2)CN(c2ccc4c(c2)C(=O)N(C2CCC(=O)NC2=O)C4)C3)C1] |
| F67 | 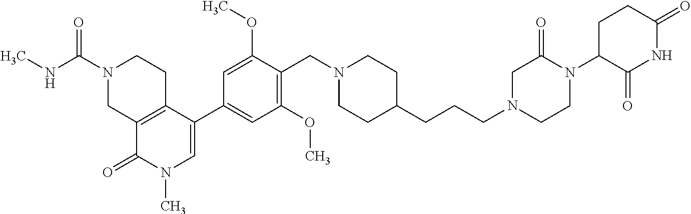 [SMILES: CNC(=O)N1CCc2c(-c3cc(OC)c(CN4CCC(CCCN5CCN(C6CCC(=O)NC6=O)C(=O)C5)CC4)c(OC)c3)cn(C)c(=O)c2C1] |
| F68 | 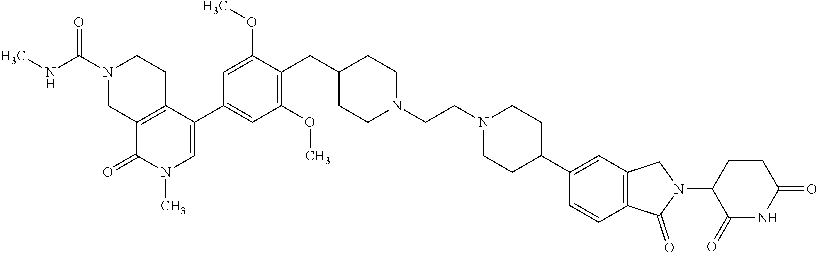 [SMILES: CNC(=O)N1CCc2c(-c3cc(OC)c(CC4CCN(CCN5CCC(c6ccc7c(c6)CN(C6CCC(=O)NC6=O)C7=O)CC5)CC4)c(OC)c3)cn(C)c(=O)c2C1] |
| F69 | 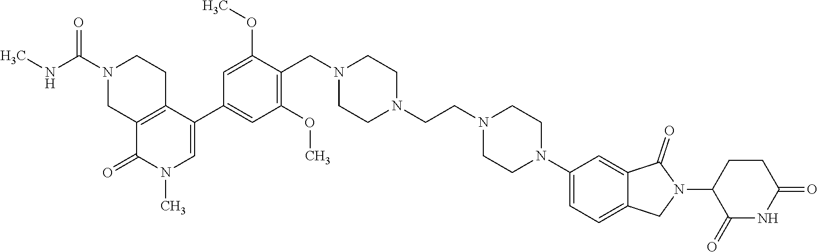 [SMILES: CNC(=O)N1CCc2c(-c3cc(OC)c(CN4CCN(CCN5CCN(c6ccc7c(c6)C(=O)N(C6CCC(=O)NC6=O)C7)CC5)CC4)c(OC)c3)cn(C)c(=O)c2C1] |
| F70 | 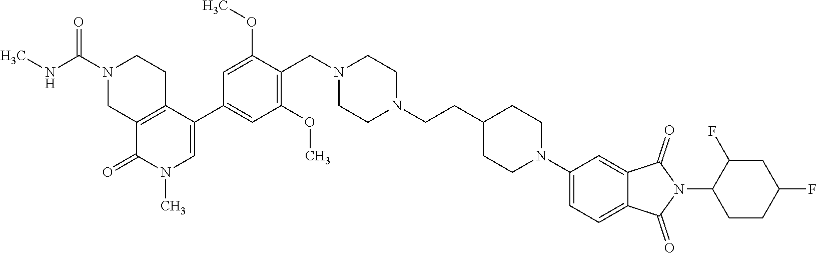 [SMILES: CNC(=O)N1CCc2c(-c3cc(OC)c(CN4CCN(CCC5CCN(c6ccc7c(c6)C(=O)N(C6CCC(F)CC6F)C7=O)CC5)CC4)c(OC)c3)cn(C)c(=O)c2C1] |
| F71 | 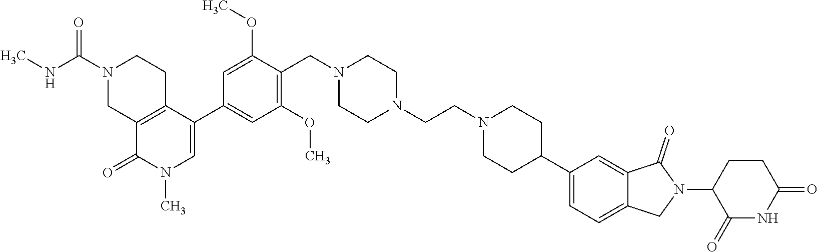 [SMILES: CNC(=O)N1CCc2c(-c3cc(OC)c(CN4CCN(CCN5CCC(c6ccc7c(c6)C(=O)N(C6CCC(=O)NC6=O)C7)CC5)CC4)c(OC)c3)cn(C)c(=O)c2C1] |
| F72 | 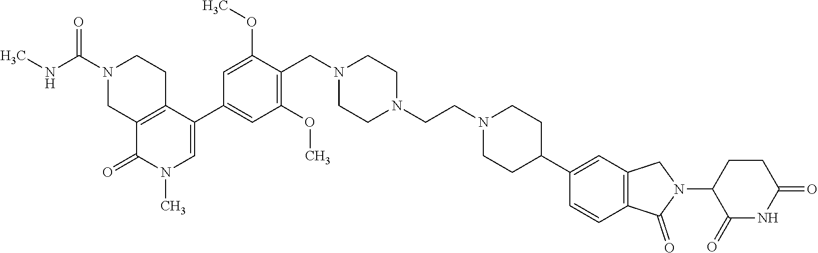 [SMILES: CNC(=O)N1CCc2c(-c3cc(OC)c(CN4CCN(CCN5CCC(c6ccc7c(c6)CN(C6CCC(=O)NC6=O)C7=O)CC5)CC4)c(OC)c3)cn(C)c(=O)c2C1] |
| F73 | 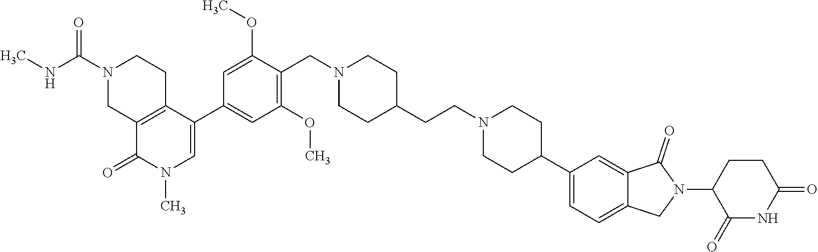 [SMILES: CNC(=O)N1CCc2c(-c3cc(OC)c(CN4CCC(CCN5CCC(c6ccc7c(c6)C(=O)N(C6CCC(=O)NC6=O)C7)CC5)CC4)c(OC)c3)cn(C)c(=O)c2C1] |
| F74 | 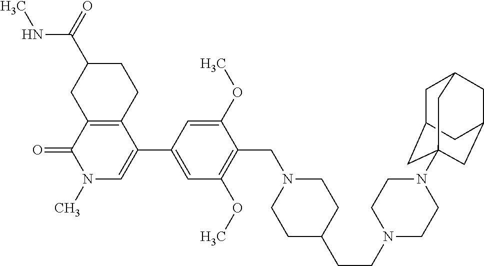 [SMILES: CNC(=O)C1CCc2c(-c3cc(OC)c(CN4CCC(CCN5CCN(C67CC8CC(CC(C8)C6)C7)CC5)CC4)c(OC)c3)cn(C)c(=O)c2C1] |
| F75 | 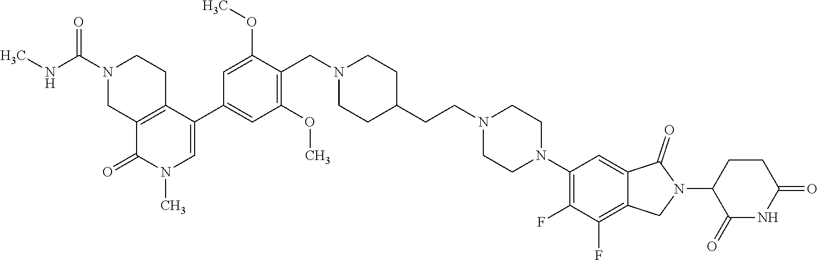 [SMILES: CNC(=O)N1CCc2c(-c3cc(OC)c(CN4CCC(CCN5CCN(c6cc7c(c(F)c6F)CN(C6CCC(=O)NC6=O)C7=O)CC5)CC4)c(OC)c3)cn(C)c(=O)c2C1] |
| F76 | 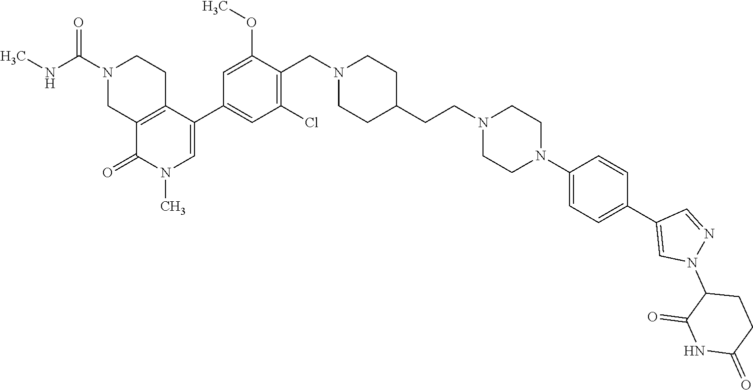 [SMILES: CNC(=O)N1CCc2c(-c3cc(Cl)c(CN4CCC(CCN5CCN(c6ccc(-c7cnn(C8CCC(=O)NC8=O)c7)cc6)CC5)CC4)c(OC)c3)cn(C)c(=O)c2C1] |
| F77 | 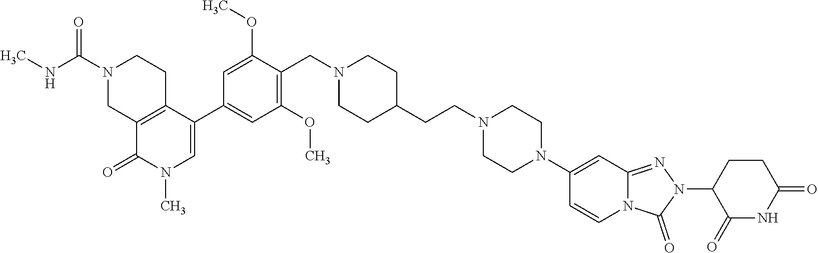 [SMILES: CNC(=O)N1CCc2c(-c3cc(OC)c(CN4CCC(CCN5CCN(c6ccn7c(=O)n(C8CCC(=O)NC8=O)nc7c6)CC5)CC4)c(OC)c3)cn(C)c(=O)c2C1] |
| F78 | 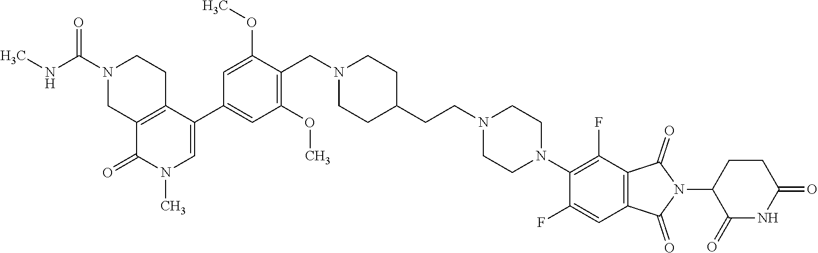 [SMILES: CNC(=O)N1CCc2c(-c3cc(OC)c(CN4CCC(CCN5CCN(c6c(F)cc7c(c6F)C(=O)N(C6CCC(=O)NC6=O)C7=O)CC5)CC4)c(OC)c3)cn(C)c(=O)c2C1] |
| F79 | 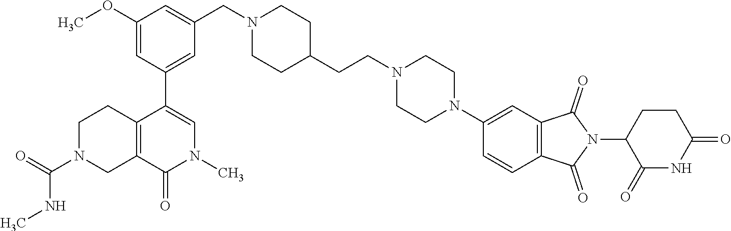 [SMILES: CNC(=O)N1CCc2c(-c3cc(CN4CCC(CCN5CCN(c6ccc7c(c6)C(=O)N(C6CCC(=O)NC6=O)C7=O)CC5)CC4)cc(OC)c3)cn(C)c(=O)c2C1] |
| F80 | 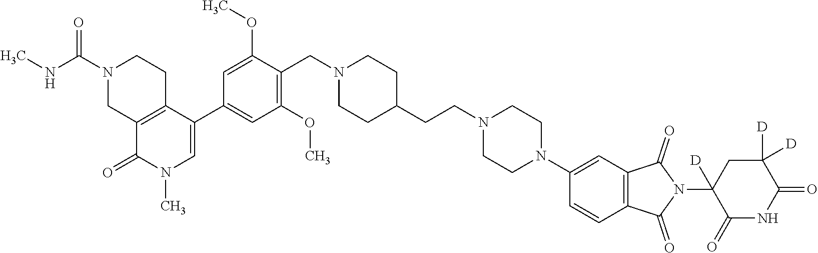 [SMILES: [2H]C1([2H])CC([2H])(N2C(=O)c3ccc(N4CCN(CCC5CCN(Cc6c(OC)cc(-c7cn(C)c(=O)c8c7CCN(C(=O)NC)C8)cc6OC)CC5)CC4)cc3C2=O)C(=O)NC1=O] |
| F81 | 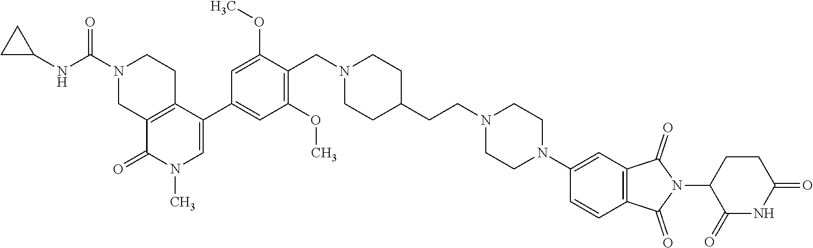 [SMILES: COc1cc(-c2cn(C)c(=O)c3c2CCN(C(=O)NC2CC2)C3)cc(OC)c1CN1CCC(CCN2CCN(c3ccc4c(c3)C(=O)N(C3CCC(=O)NC3=O)C4=O)CC2)CC1] |
| F82 | 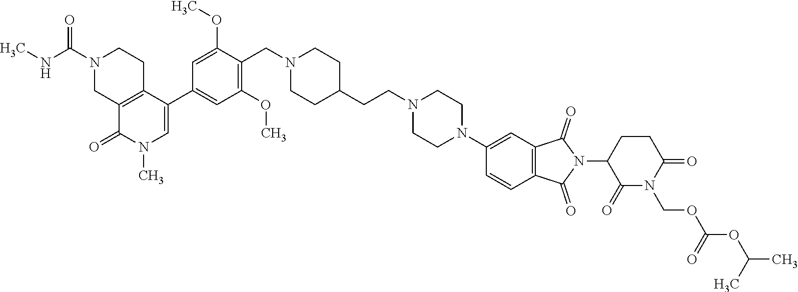 [SMILES: CNC(=O)N1CCc2c(-c3cc(OC)c(CN4CCC(CCN5CCN(c6ccc7c(c6)C(=O)N(C6CCC(=O)N(COC(=O)OC(C)C)C6=O)C7=O)CC5)CC4)c(OC)c3)cn(C)c(=O)c2C1] |
| F83 | 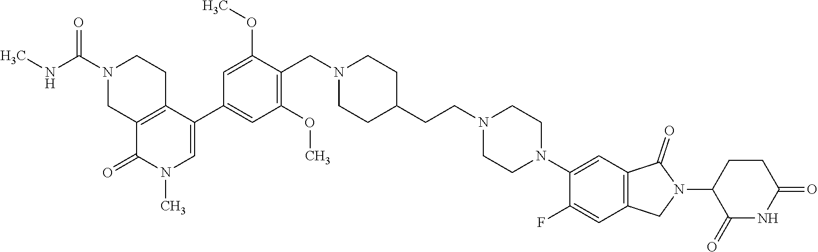 [SMILES: CNC(=O)N1CCc2c(-c3cc(OC)c(CN4CCC(CCN5CCN(c6cc7c(cc6F)CN(C6CCC(=O)NC6=O)C7=O)CC5)CC4)c(OC)c3)cn(C)c(=O)c2C1] |
| F84 | 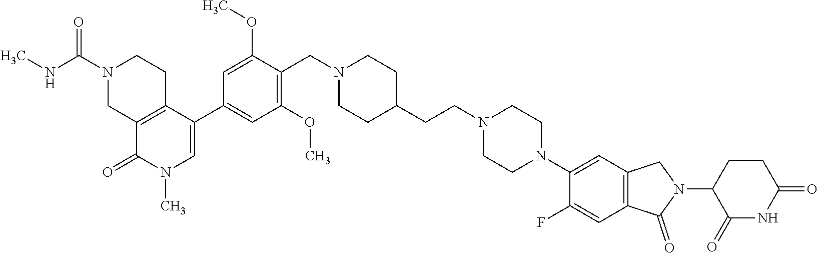 [SMILES: CNC(=O)N1CCc2c(-c3cc(OC)c(CN4CCC(CCN5CCN(c6cc7c(cc6F)C(=O)N(C6CCC(=O)NC6=O)C7)CC5)CC4)c(OC)c3)cn(C)c(=O)c2C1] |
| F85 | 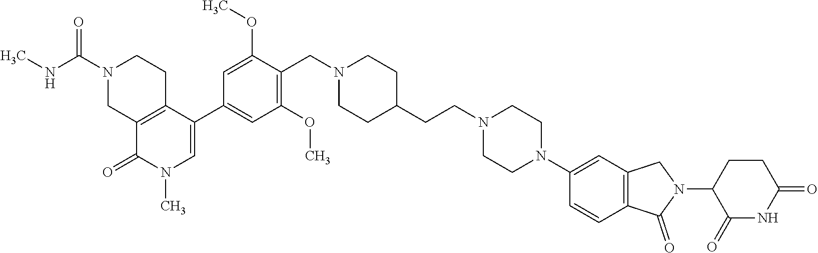 [SMILES: CNC(=O)N1CCc2c(-c3cc(OC)c(CN4CCC(CCN5CCN(c6ccc7c(c6)CN(C6CCC(=O)NC6=O)C7=O)CC5)CC4)c(OC)c3)cn(C)c(=O)c2C1] |
| F86 | 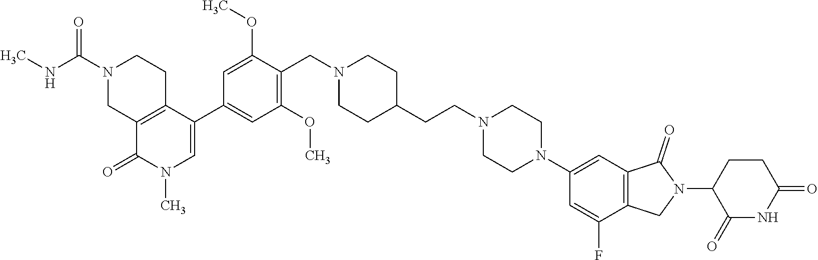 [SMILES: CNC(=O)N1CCc2c(-c3cc(OC)c(CN4CCC(CCN5CCN(c6cc(F)c7c(c6)C(=O)N(C6CCC(=O)NC6=O)C7)CC5)CC4)c(OC)c3)cn(C)c(=O)c2C1] |
| F87 | 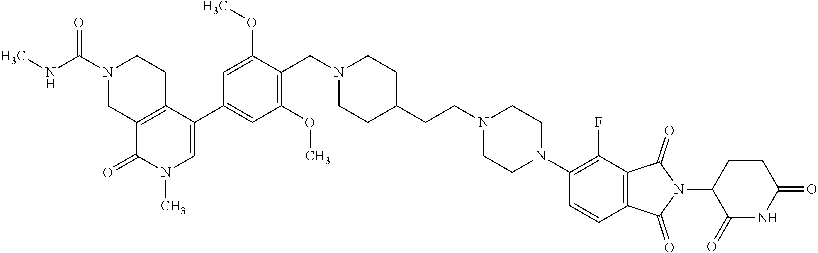 [SMILES: CNC(=O)N1CCc2c(-c3cc(OC)c(CN4CCC(CCN5CCN(c6ccc7c(c6F)C(=O)N(C6CCC(=O)NC6=O)C7=O)CC5)CC4)c(OC)c3)cn(C)c(=O)c2C1] |
| F88 | 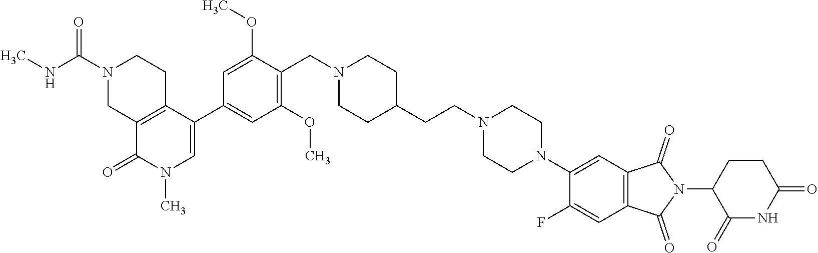 [SMILES: CNC(=O)N1CCc2c(-c3cc(OC)c(CN4CCC(CCN5CCN(c6cc7c(cc6F)C(=O)N(C6CCC(=O)NC6=O)C7=O)CC5)CC4)c(OC)c3)cn(C)c(=O)c2C1] |
| F89 | 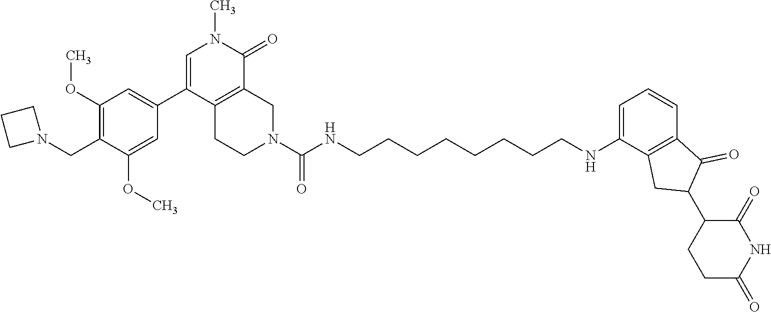 [SMILES: COc1cc(-c2cn(C)c(=O)c3c2CCN(C(=O)NCCCCCCCCNc2cccc4c2CC(C2CCC(=O)NC2=O)C4=O)C3)cc(OC)c1CN1CCC1] |
| F90 | 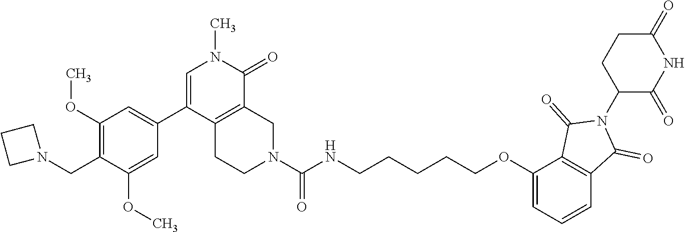 [SMILES: COc1cc(-c2cn(C)c(=O)c3c2CCN(C(=O)NCCCCCOc2cccc4c2C(=O)N(C2CCC(=O)NC2=O)C4=O)C3)cc(OC)c1CN1CCC1] |
| F91 | 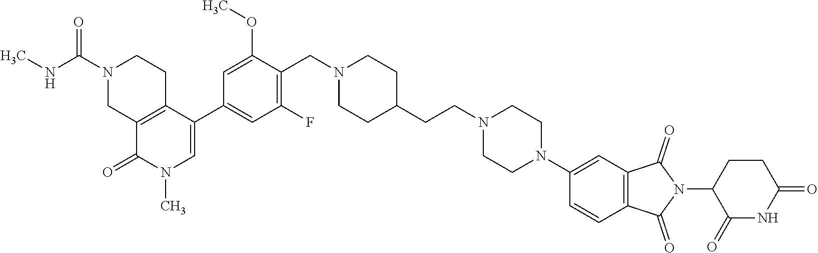 [SMILES: CNC(=O)N1CCc2c(-c3cc(F)c(CN4CCC(CCN5CCN(c6ccc7c(c6)C(=O)N(C6CCC(=O)NC6=O)C7=O)CC5)CC4)c(OC)c3)cn(C)c(=O)c2C1] |
| F92 | 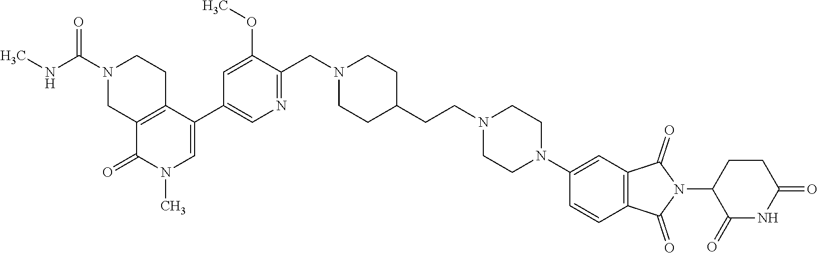 [SMILES: CNC(=O)N1CCc2c(-c3cnc(CN4CCC(CCN5CCN(c6ccc7c(c6)C(=O)N(C6CCC(=O)NC6=O)C7=O)CC5)CC4)c(OC)c3)cn(C)c(=O)c2C1] |
| F93 | 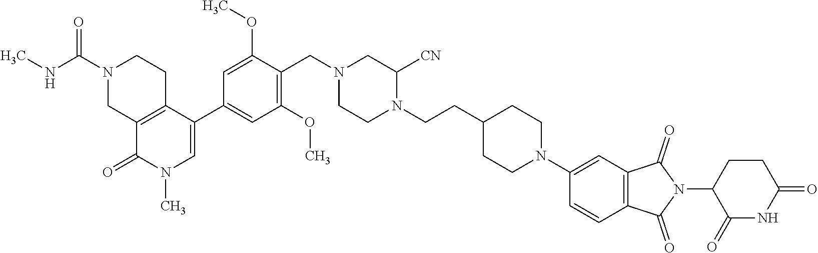 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)C(C#N)C4)c(OC)c3)cn(C)c(=O)c2C1] |
| F94 | 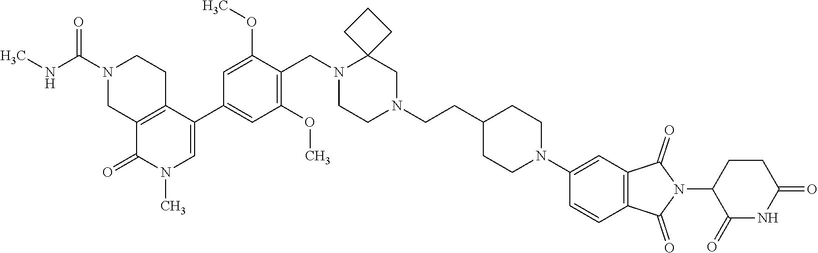 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)CC45CCC5)c(OC)c3)cn(C)c(=O)c2C1] |
| F95 | 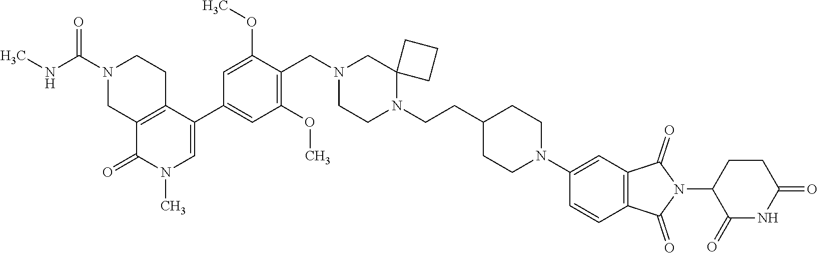 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)C5(CCC5)C4)c(OC)c3)cn(C)c(=O)c2C1] |
| F96 | 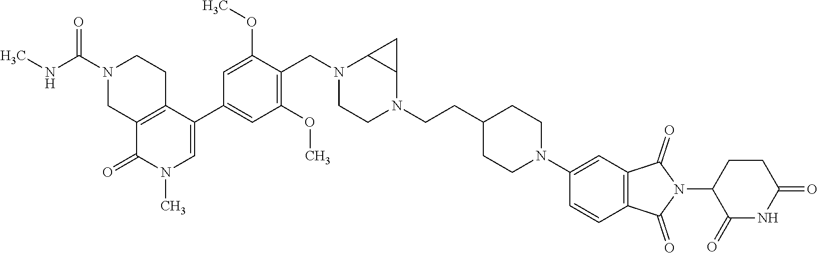 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)C5CC54)c(OC)c3)cn(C)c(=O)c2C1] |
| F97 | 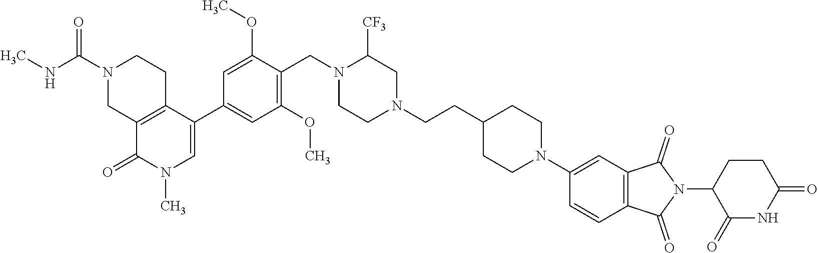 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)CC4C(F)(F)F)c(OC)c3)cn(C)c(=O)c2C1] |
| F98 | 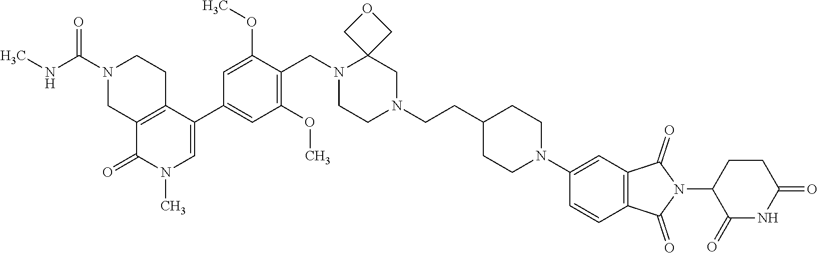 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)CC45COC5)c(OC)c3)cn(C)c(=O)c2C1] |
| F99 | 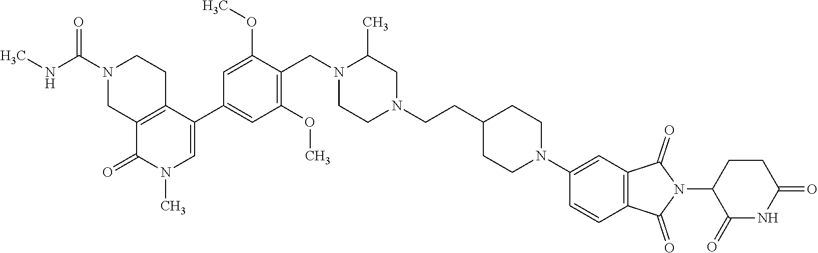 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)CC4C)c(OC)c3)cn(C)c(=O)c2C1] |
| F100 | 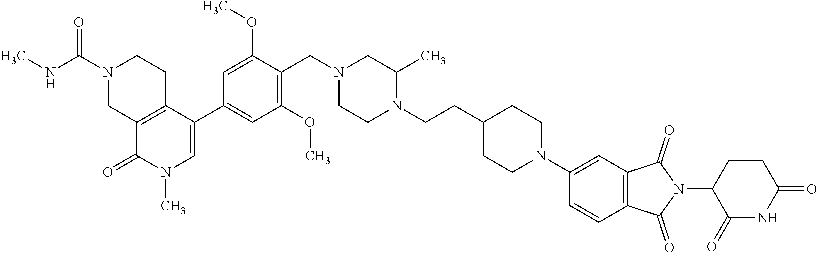 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)C(C)C4)c(OC)c3)cn(C)c(=O)c2C1] |
| F101 | 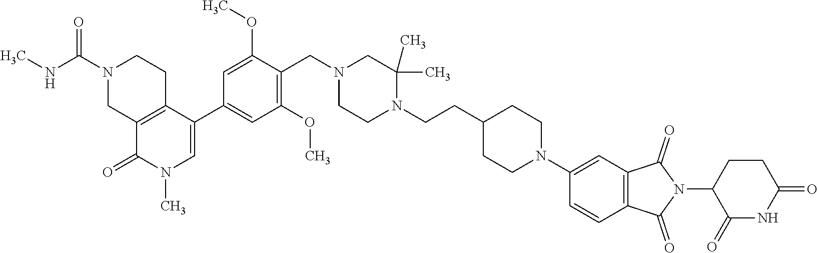 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)C(C)(C)C4)c(OC)c3)cn(C)c(=O)c2C1] |
| F102 | 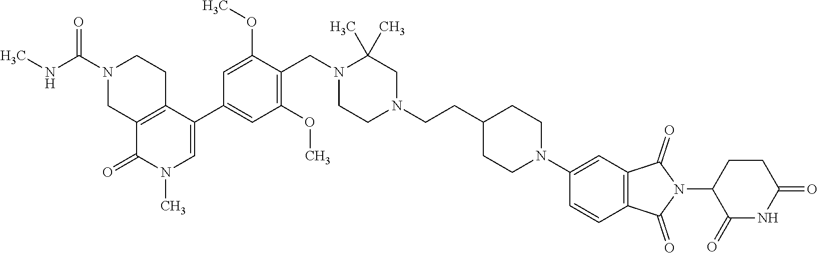 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)CC4(C)C)c(OC)c3)cn(C)c(=O)c2C1] |
| F103 | 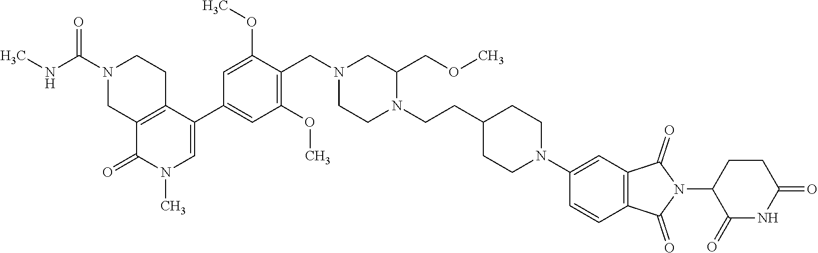 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)C(COC)C4)c(OC)c3)cn(C)c(=O)c2C1] |
| F104 | 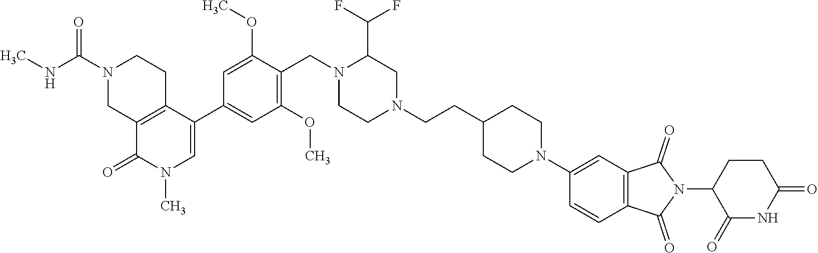 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)CC4C(F)F)c(OC)c3)cn(C)c(=O)c2C1] |
| F105 | 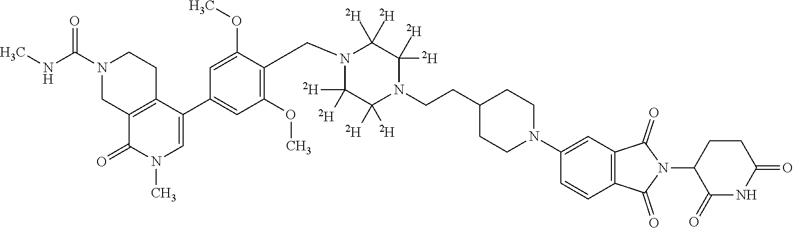 [SMILES: [2H]C1([2H])N(CCC2CCN(c3ccc4c(c3)C(=O)N(C3CCC(=O)NC3=O)C4=O)CC2)C([2H])([2H])C([2H])([2H])N(Cc2c(OC)cc(-c3cn(C)c(=O)c4c3CCN(C(=O)NC)C4)cc2OC)C1([2H])[2H]] |
| F106 | 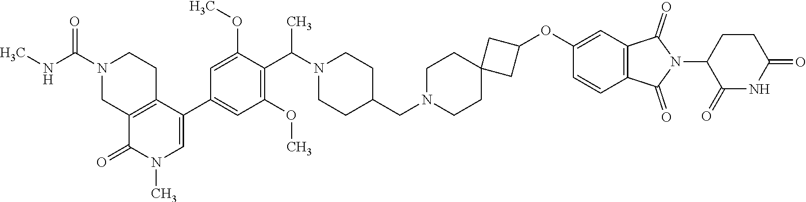 [SMILES: CNC(=O)N1CCc2c(-c3cc(OC)c(C(C)N4CCC(CN5CCC6(CC5)CC(Oc5ccc7c(c5)C(=O)N(C5CCC(=O)NC5=O)C7=O)C6)CC4)c(OC)c3)cn(C)c(=O)c2C1] |
| F107 | 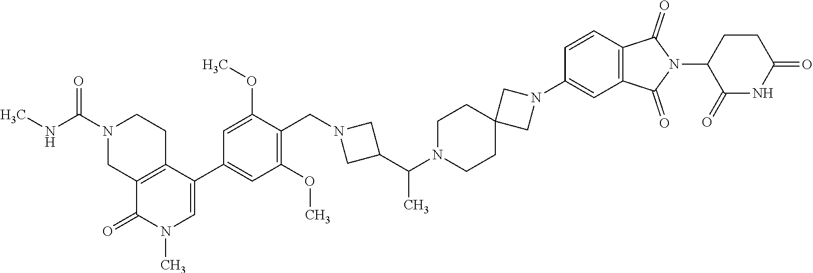 [SMILES: CNC(=O)N1CCc2c(-c3cc(OC)c(CN4CC(C(C)N5CCC6(CC5)CN(c5ccc7c(c5)C(=O)N(C5CCC(=O)NC5=O)C7=O)C6)C4)c(OC)c3)cn(C)c(=O)c2C1] |
| F108 | 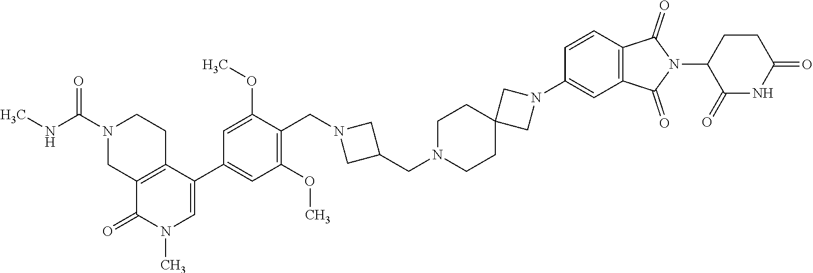 [SMILES: CNC(=O)N1CCc2c(-c3cc(OC)c(CN4CC(CN5CCC6(CC5)CN(c5ccc7c(c5)C(=O)N(C5CCC(=O)NC5=O)C7=O)C6)C4)c(OC)c3)cn(C)c(=O)c2C1] |
| F109 | 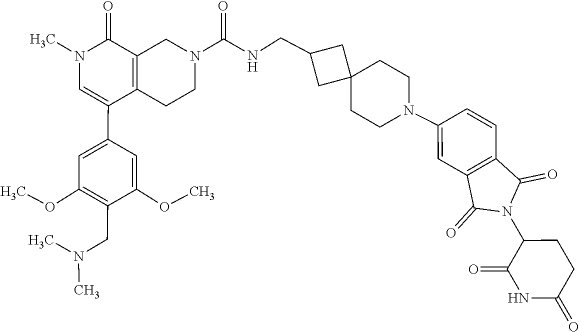 [SMILES: COc1cc(-c2cn(C)c(=O)c3c2CCN(C(=O)NCC2CC4(CCN(c5ccc6c(c5)C(=O)N(C5CCC(=O)NC5=O)C6=O)CC4)C2)C3)cc(OC)c1CN(C)C] |
| F110 | 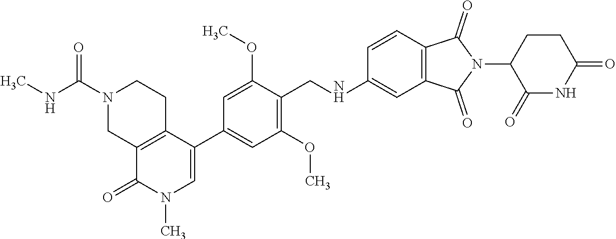 [SMILES: CNC(=O)N1CCc2c(-c3cc(OC)c(CNc4ccc5c(c4)C(=O)N(C4CCC(=O)NC4=O)C5=O)c(OC)c3)cn(C)c(=O)c2C1] |
| F111 | 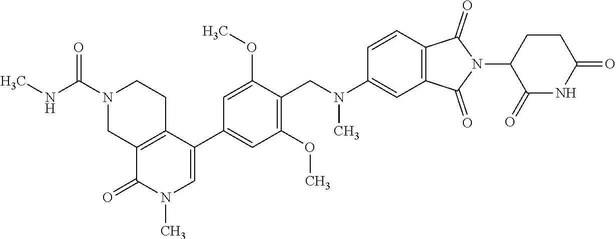 [SMILES: CNC(=O)N1CCc2c(-c3cc(OC)c(CN(C)c4ccc5c(c4)C(=O)N(C4CCC(=O)NC4=O)C5=O)c(OC)c3)cn(C)c(=O)c2C1] |
| F112 | 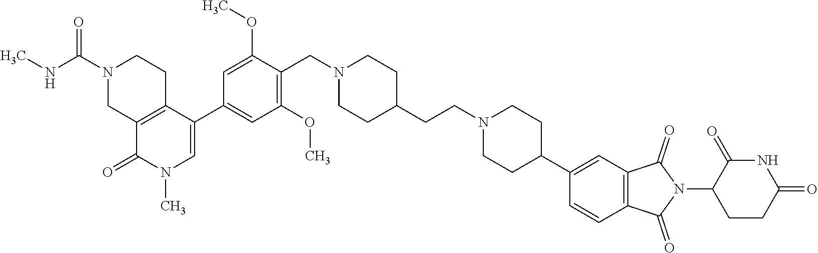 [SMILES: CNC(=O)N1CCc2c(-c3cc(OC)c(CN4CCC(CCN5CCC(c6ccc7c(c6)C(=O)N(C6CCC(=O)NC6=O)C7=O)CC5)CC4)c(OC)c3)cn(C)c(=O)c2C1] |
| F113 | 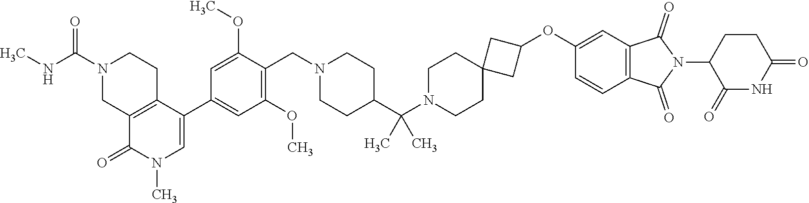 [SMILES: CNC(=O)N1CCc2c(-c3cc(OC)c(CN4CCC(C(C)(C)N5CCC6(CC5)CC(Oc5ccc7c(c5)C(=O)N(C5CCC(=O)NC5=O)C7=O)C6)CC4)c(OC)c3)cn(C)c(=O)c2C1] |
| F114 | 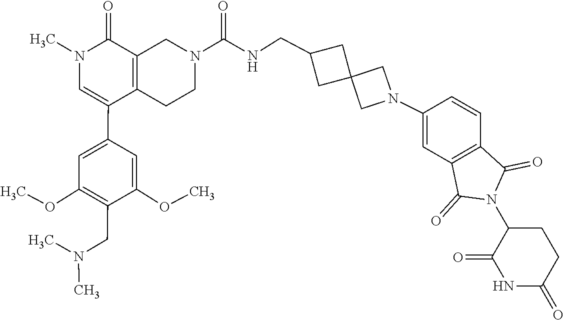 [SMILES: COc1cc(-c2cn(C)c(=O)c3c2CCN(C(=O)NCC2CC4(C2)CN(c2ccc5c(c2)C(=O)N(C2CCC(=O)NC2=O)C5=O)C4)C3)cc(OC)c1CN(C)C] |
| F115 | 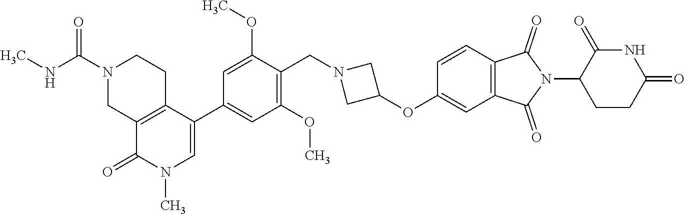 [SMILES: CNC(=O)N1CCc2c(-c3cc(OC)c(CN4CC(Oc5ccc6c(c5)C(=O)N(C5CCC(=O)NC5=O)C6=O)C4)c(OC)c3)cn(C)c(=O)c2C1] |
| F116 | 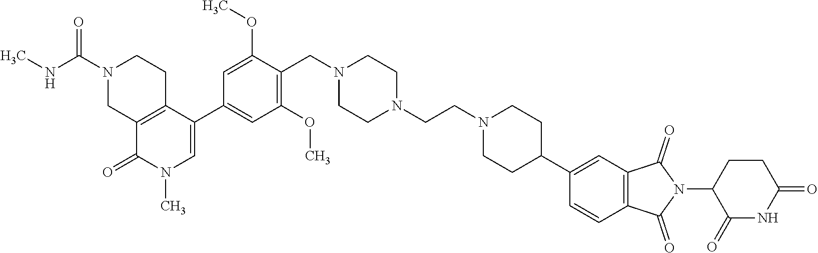 [SMILES: CNC(=O)N1CCc2c(-c3cc(OC)c(CN4CCN(CCN5CCC(c6ccc7c(c6)C(=O)N(C6CCC(=O)NC6=O)C7=O)CC5)CC4)c(OC)c3)cn(C)c(=O)c2C1] |
| F117 | 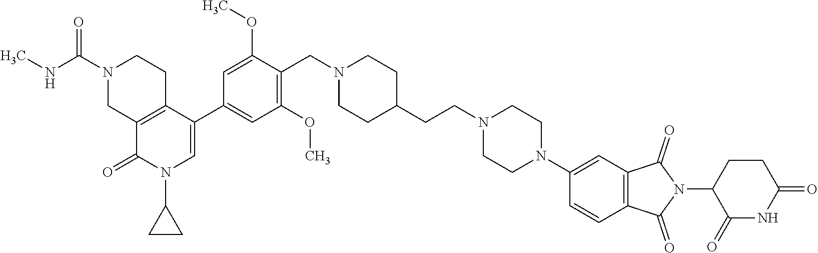 [SMILES: CNC(=O)N1CCc2c(-c3cc(OC)c(CN4CCC(CCN5CCN(c6ccc7c(c6)C(=O)N(C6CCC(=O)NC6=O)C7=O)CC5)CC4)c(OC)c3)cn(C3CC3)c(=O)c2C1] |
| F118 | 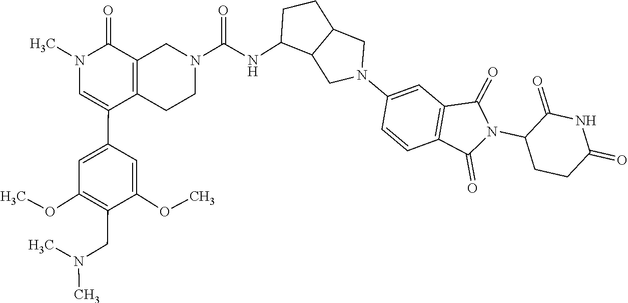 [SMILES: COc1cc(-c2cn(C)c(=O)c3c2CCN(C(=O)NC2CCC4CN(c5ccc6c(c5)C(=O)N(C5CCC(=O)NC5=O)C6=O)CC42)C3)cc(OC)c1CN(C)C] |
| F119 | 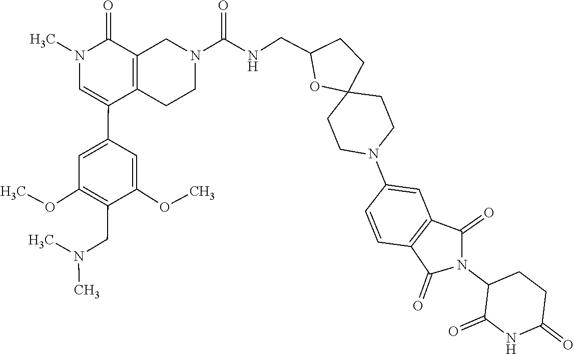 [SMILES: COc1cc(-c2cn(C)c(=O)c3c2CCN(C(=O)NCC2CCC4(CCN(c5ccc6c(c5)C(=O)N(C5CCC(=O)NC5=O)C6=O)CC4)O2)C3)cc(OC)c1CN(C)C] |
| F120 | 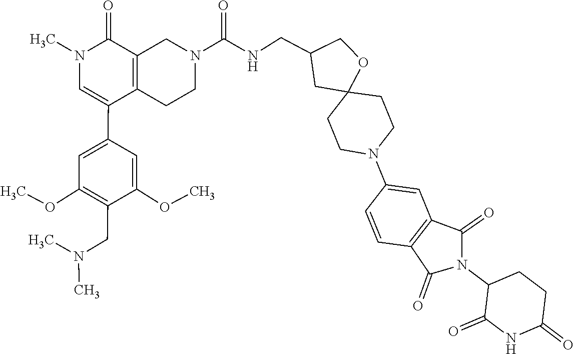 [SMILES: COc1cc(-c2cn(C)c(=O)c3c2CCN(C(=O)NCC2COC4(CCN(c5ccc6c(c5)C(=O)N(C5CCC(=O)NC5=O)C6=O)CC4)C2)C3)cc(OC)c1CN(C)C] |
| F121 | 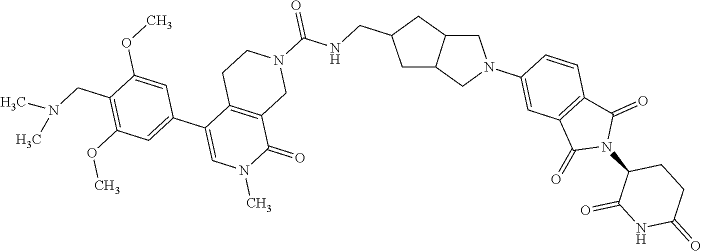 [SMILES: COc1cc(-c2cn(C)c(=O)c3c2CCN(C(=O)NCC2CC4CN(c5ccc6c(c5)C(=O)N([C@H]5CCC(=O)NC5=O)C6=O)CC4C2)C3)cc(OC)c1CN(C)C] |
| F122 | 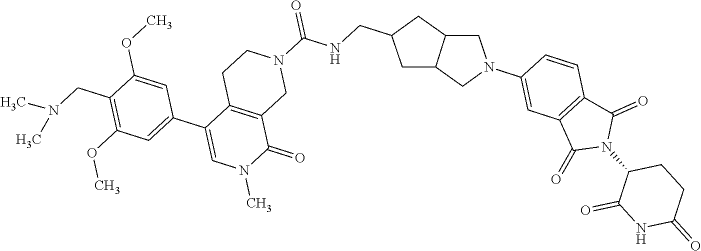 [SMILES: COc1cc(-c2cn(C)c(=O)c3c2CCN(C(=O)NCC2CC4CN(c5ccc6c(c5)C(=O)N([C@@H]5CCC(=O)NC5=O)C6=O)CC4C2)C3)cc(OC)c1CN(C)C] |
| F123 | 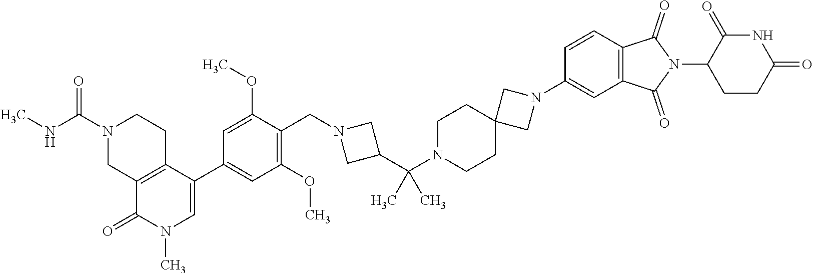 [SMILES: CNC(=O)N1CCc2c(-c3cc(OC)c(CN4CC(C(C)(C)N5CCC6(CC5)CN(c5ccc7c(c5)C(=O)N(C5CCC(=O)NC5=O)C7=O)C6)C4)c(OC)c3)cn(C)c(=O)c2C1] |
| F124 | 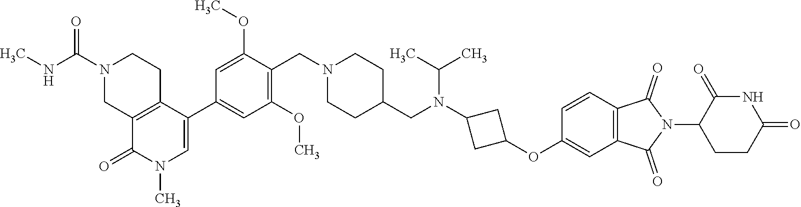 [SMILES: CNC(=O)N1CCc2c(-c3cc(OC)c(CN4CCC(CN(C(C)C)C5CC(Oc6ccc7c(c6)C(=O)N(C6CCC(=O)NC6=O)C7=O)C5)CC4)c(OC)c3)cn(C)c(=O)c2C1] |
| F125 | 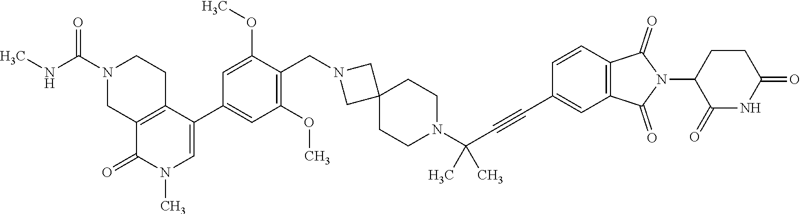 [SMILES: CNC(=O)N1CCc2c(-c3cc(OC)c(CN4CC5(CCN(C(C)(C)C#Cc6ccc7c(c6)C(=O)N(C6CCC(=O)NC6=O)C7=O)CC5)C4)c(OC)c3)cn(C)c(=O)c2C1] |
| F126 | 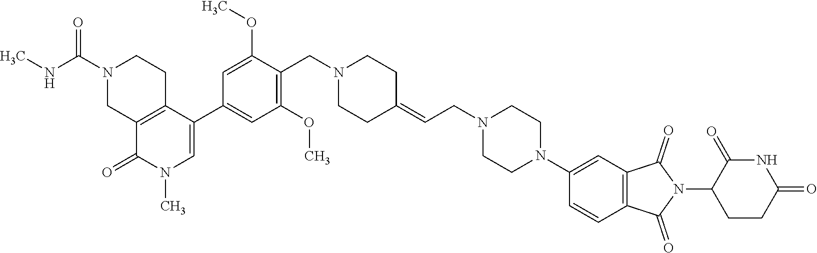 [SMILES: CNC(=O)N1CCc2c(-c3cc(OC)c(CN4CCC(=CCN5CCN(c6ccc7c(c6)C(=O)N(C6CCC(=O)NC6=O)C7=O)CC5)CC4)c(OC)c3)cn(C)c(=O)c2C1] |
| F127 | 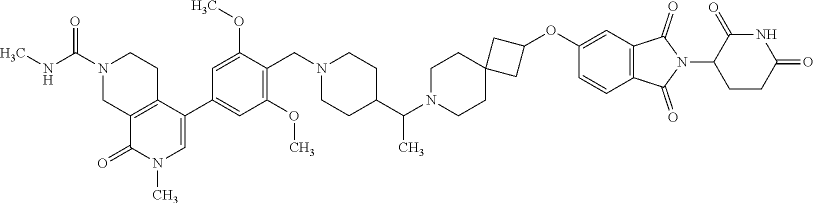 [SMILES: CNC(=O)N1CCc2c(-c3cc(OC)c(CN4CCC(C(C)N5CCC6(CC5)CC(Oc5ccc7c(c5)C(=O)N(C5CCC(=O)NC5=O)C7=O)C6)CC4)c(OC)c3)cn(C)c(=O)c2C1] |
| F128 | 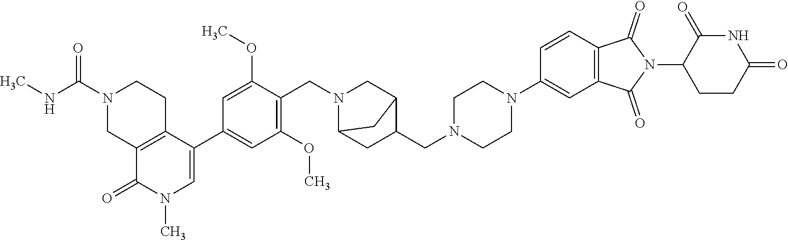 [SMILES: CNC(=O)N1CCc2c(-c3cc(OC)c(CN4CC5CC4CC5CN4CCN(c5ccc6c(c5)C(=O)N(C5CCC(=O)NC5=O)C6=O)CC4)c(OC)c3)cn(C)c(=O)c2C1] |
| F129 | 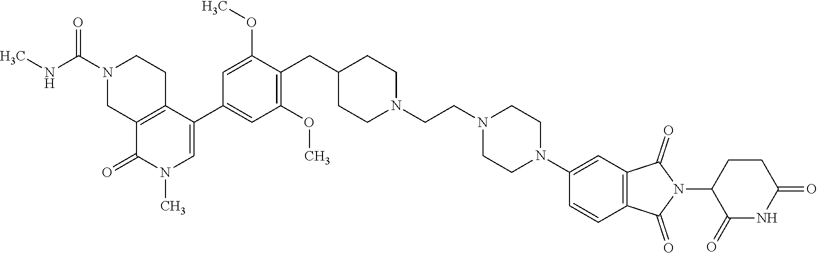 [SMILES: CNC(=O)N1CCc2c(-c3cc(OC)c(CC4CCN(CCN5CCN(c6ccc7c(c6)C(=O)N(C6CCC(=O)NC6=O)C7=O)CC5)CC4)c(OC)c3)cn(C)c(=O)c2C1] |
| F130 | 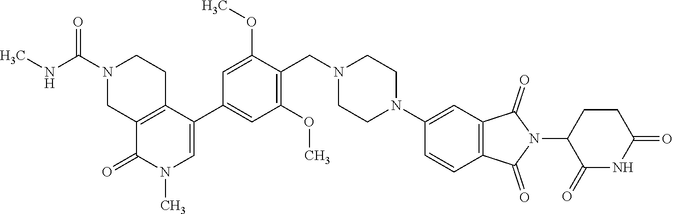 [SMILES: CNC(=O)N1CCc2c(-c3cc(OC)c(CN4CCN(c5ccc6c(c5)C(=O)N(C5CCC(=O)NC5=O)C6=O)CC4)c(OC)c3)cn(C)c(=O)c2C1] |
| F131 | 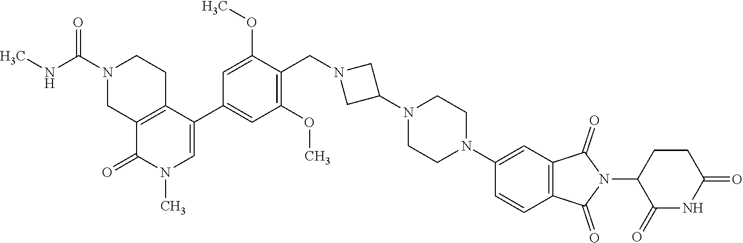 [SMILES: CNC(=O)N1CCc2c(-c3cc(OC)c(CN4CC(N5CCN(c6ccc7c(c6)C(=O)N(C6CCC(=O)NC6=O)C7=O)CC5)C4)c(OC)c3)cn(C)c(=O)c2C1] |
| F132 | 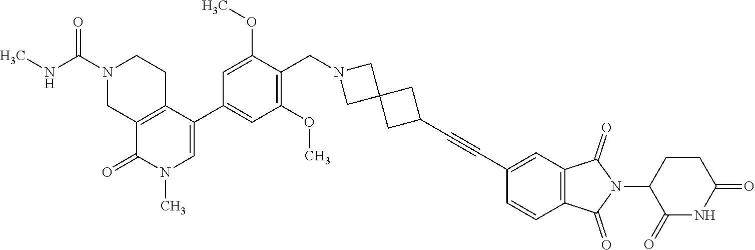 [SMILES: CNC(=O)N1CCc2c(-c3cc(OC)c(CN4CC5(CC(C#Cc6ccc7c(c6)C(=O)N(C6CCC(=O)NC6=O)C7=O)C5)C4)c(OC)c3)cn(C)c(=O)c2C1] |
| F133 | 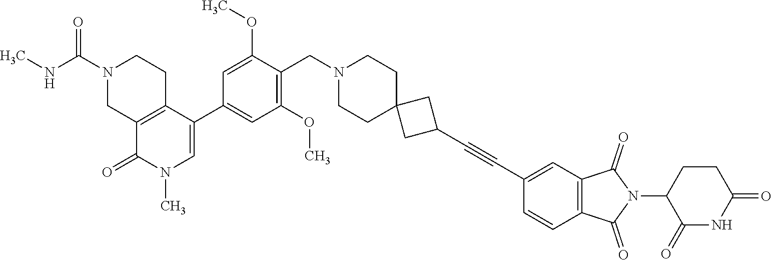 [SMILES: CNC(=O)N1CCc2c(-c3cc(OC)c(CN4CCC5(CC4)CC(C#Cc4ccc6c(c4)C(=O)N(C4CCC(=O)NC4=O)C6=O)C5)c(OC)c3)cn(C)c(=O)c2C1] |
| F134 | 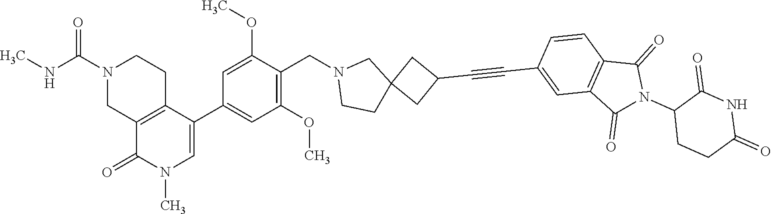 [SMILES: CNC(=O)N1CCc2c(-c3cc(OC)c(CN4CCC5(CC(C#Cc6ccc7c(c6)C(=O)N(C6CCC(=O)NC6=O)C7=O)C5)C4)c(OC)c3)cn(C)c(=O)c2C1] |
| F135 | 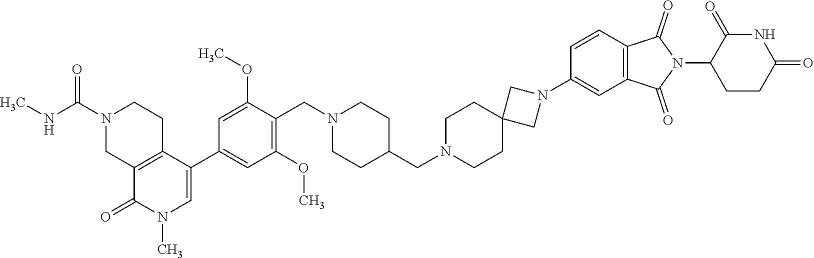 [SMILES: CNC(=O)N1CCc2c(-c3cc(OC)c(CN4CCC(CN5CCC6(CC5)CN(c5ccc7c(c5)C(=O)N(C5CCC(=O)NC5=O)C7=O)C6)CC4)c(OC)c3)cn(C)c(=O)c2C1] |
| F136 | 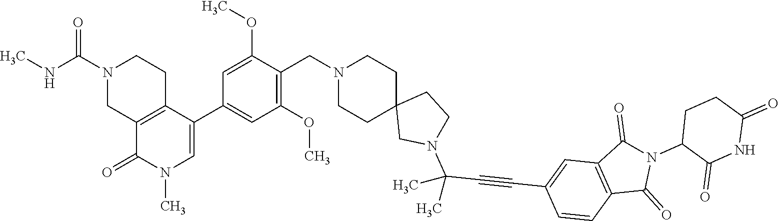 [SMILES: CNC(=O)N1CCc2c(-c3cc(OC)c(CN4CCC5(CC4)CCN(C(C)(C)C#Cc4ccc6c(c4)C(=O)N(C4CCC(=O)NC4=O)C6=O)C5)c(OC)c3)cn(C)c(=O)c2C1] |
| F137 | 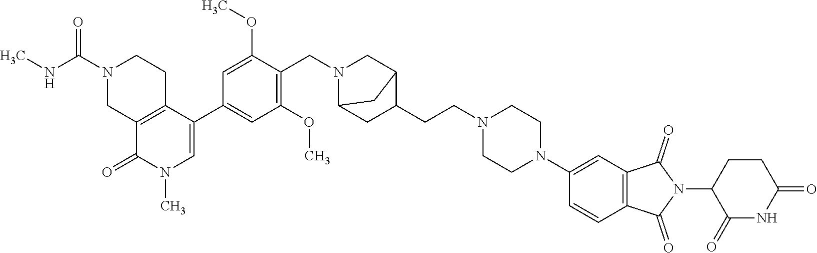 [SMILES: CNC(=O)N1CCc2c(-c3cc(OC)c(CN4CC5CC4CC5CCN4CCN(c5ccc6c(c5)C(=O)N(C5CCC(=O)NC5=O)C6=O)CC4)c(OC)c3)cn(C)c(=O)c2C1] |
| F138 | 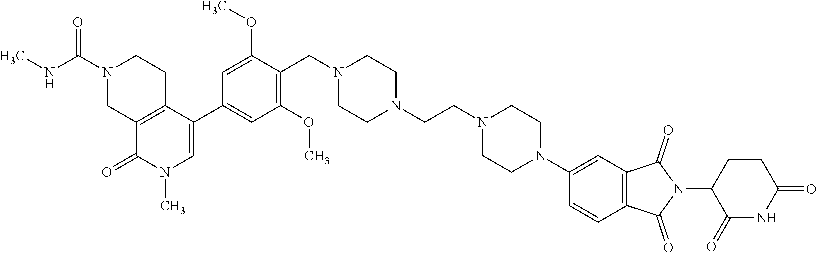 [SMILES: CNC(=O)N1CCc2c(-c3cc(OC)c(CN4CCN(CCN5CCN(c6ccc7c(c6)C(=O)N(C6CCC(=O)NC6=O)C7=O)CC5)CC4)c(OC)c3)cn(C)c(=O)c2C1] |
| F139 | 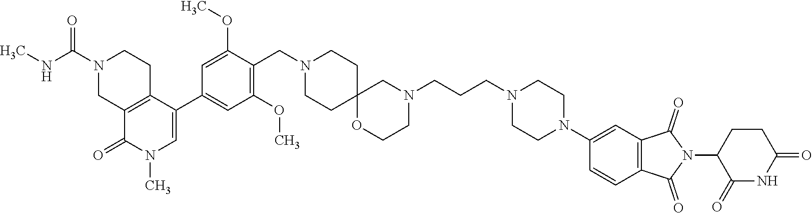 [SMILES: CNC(=O)N1CCc2c(-c3cc(OC)c(CN4CCC5(CC4)CN(CCCN4CCN(c6ccc7c(c6)C(=O)N(C6CCC(=O)NC6=O)C7=O)CC4)CCO5)c(OC)c3)cn(C)c(=O)c2C1] |
| F140 | 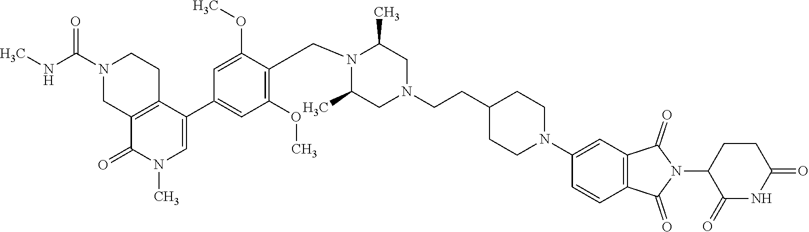 [SMILES: CNC(=O)N1CCc2c(-c3cc(OC)c(CN4[C@H](C)CN(CCC5CCN(c6ccc7c(c6)C(=O)N(C6CCC(=O)NC6=O)C7=O)CC5)C[C@@H]4C)c(OC)c3)cn(C)c(=O)c2C1] |
| F141 | 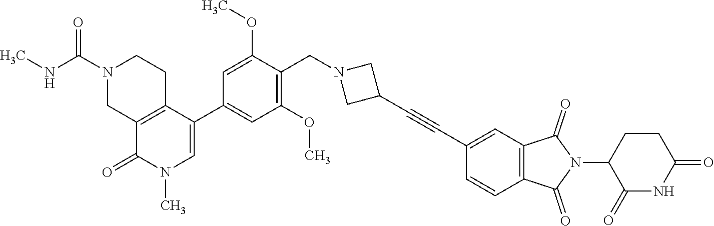 [SMILES: CNC(=O)N1CCc2c(-c3cc(OC)c(CN4CC(C#Cc5ccc6c(c5)C(=O)N(C5CCC(=O)NC5=O)C6=O)C4)c(OC)c3)cn(C)c(=O)c2C1] |
| F142 | 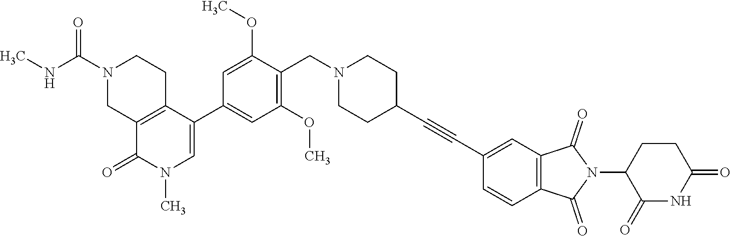 [SMILES: CNC(=O)N1CCc2c(-c3cc(OC)c(CN4CCC(C#Cc5ccc6c(c5)C(=O)N(C5CCC(=O)NC5=O)C6=O)CC4)c(OC)c3)cn(C)c(=O)c2C1] |
| F143 | 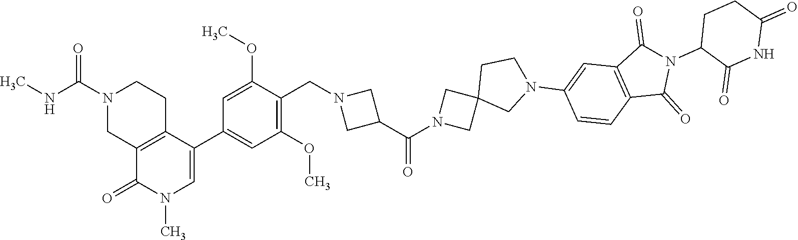 [SMILES: CNC(=O)N1CCc2c(-c3cc(OC)c(CN4CC(C(=O)N5CC6(CCN(c7ccc8c(c7)C(=O)N(C7CCC(=O)NC7=O)C8=O)C6)C5)C4)c(OC)c3)cn(C)c(=O)c2C1] |
| F144 | 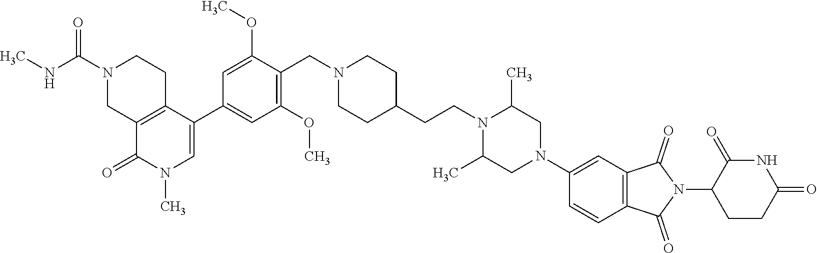 [SMILES: CNC(=O)N1CCc2c(-c3cc(OC)c(CN4CCC(CCN5C(C)CN(c6ccc7c(c6)C(=O)N(C6CCC(=O)NC6=O)C7=O)CC5C)CC4)c(OC)c3)cn(C)c(=O)c2C1] |
| F145 | 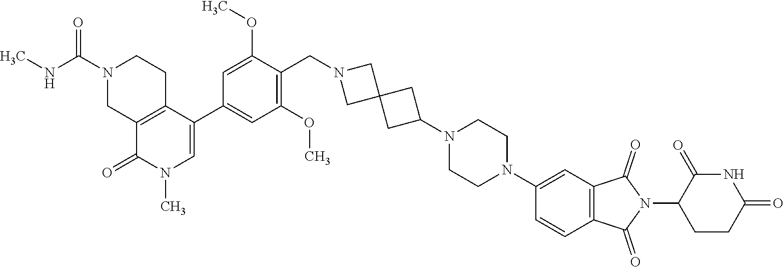 [SMILES: CNC(=O)N1CCc2c(-c3cc(OC)c(CN4CC5(CC(N6CCN(c7ccc8c(c7)C(=O)N(C7CCC(=O)NC7=O)C8=O)CC6)C5)C4)c(OC)c3)cn(C)c(=O)c2C1] |
| F146 | 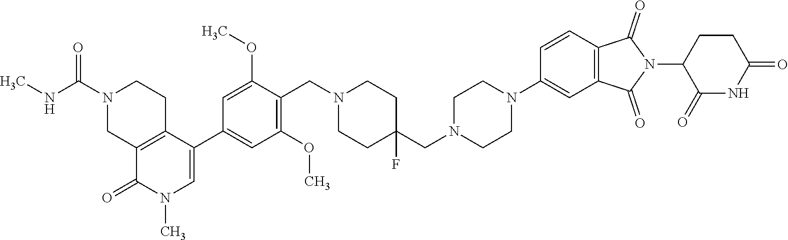 [SMILES: CNC(=O)N1CCc2c(-c3cc(OC)c(CN4CCC(F)(CN5CCN(c6ccc7c(c6)C(=O)N(C6CCC(=O)NC6=O)C7=O)CC5)CC4)c(OC)c3)cn(C)c(=O)c2C1] |
| F147 | 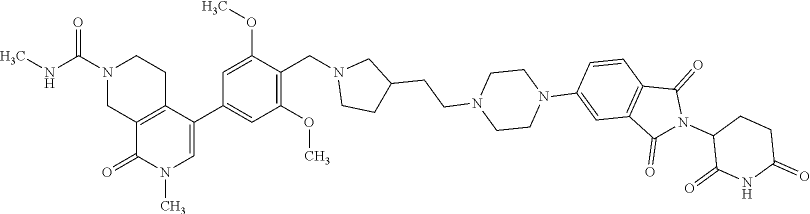 [SMILES: CNC(=O)N1CCc2c(-c3cc(OC)c(CN4CCC(CCN5CCN(c6ccc7c(c6)C(=O)N(C6CCC(=O)NC6=O)C7=O)CC5)C4)c(OC)c3)cn(C)c(=O)c2C1] |
| F148 | 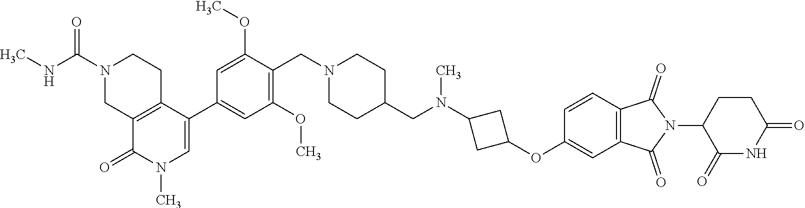 [SMILES: CNC(=O)N1CCc2c(-c3cc(OC)c(CN4CCC(CN(C)C5CC(Oc6ccc7c(c6)C(=O)N(C6CCC(=O)NC6=O)C7=O)C5)CC4)c(OC)c3)cn(C)c(=O)c2C1] |
| F149 | 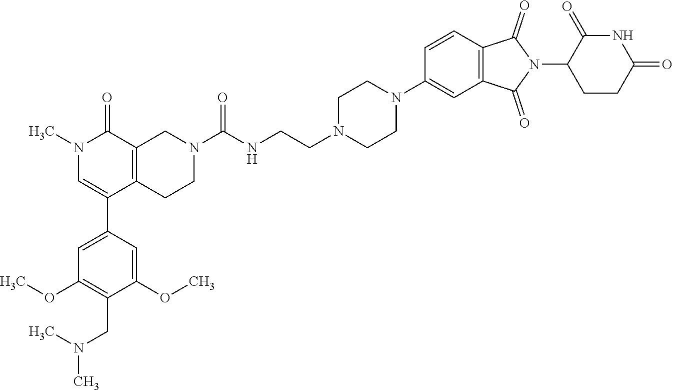 [SMILES: COc1cc(-c2cn(C)c(=O)c3c2CCN(C(=O)NCCN2CCN(c4ccc5c(c4)C(=O)N(C4CCC(=O)NC4=O)C5=O)CC2)C3)cc(OC)c1CN(C)C] |
| F150 | 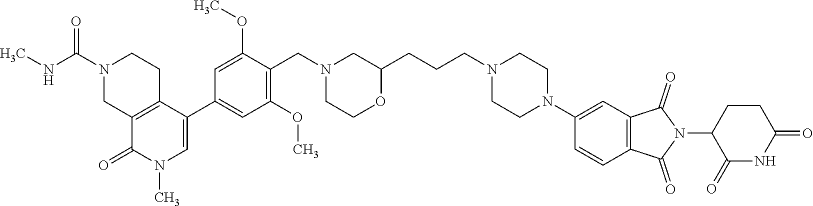 [SMILES: CNC(=O)N1CCc2c(-c3cc(OC)c(CN4CCOC(CCCN5CCN(c6ccc7c(c6)C(=O)N(C6CCC(=O)NC6=O)C7=O)CC5)C4)c(OC)c3)cn(C)c(=O)c2C1] |
| F151 | 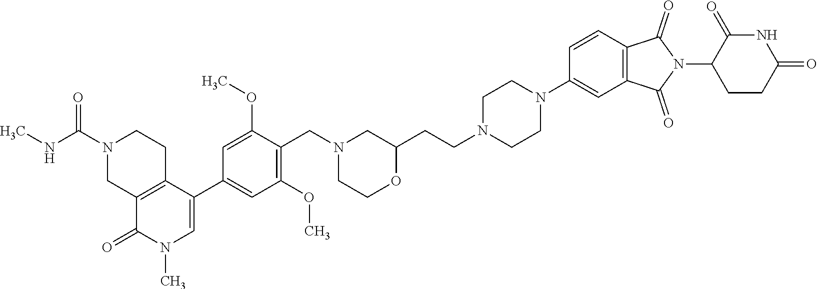 [SMILES: CNC(=O)N1CCc2c(-c3cc(OC)c(CN4CCOC(CCN5CCN(c6ccc7c(c6)C(=O)N(C6CCC(=O)NC6=O)C7=O)CC5)C4)c(OC)c3)cn(C)c(=O)c2C1] |
| F152 | 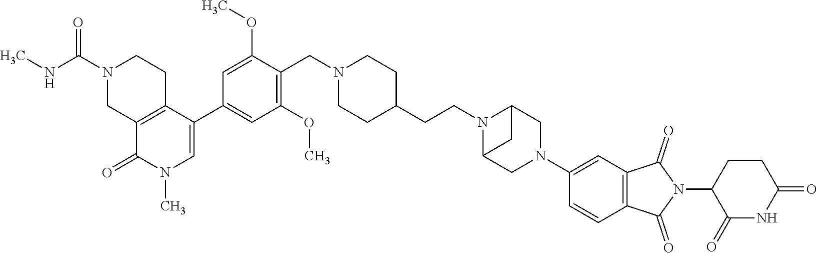 [SMILES: CNC(=O)N1CCc2c(-c3cc(OC)c(CN4CCC(CCN5C6CC5CN(c5ccc7c(c5)C(=O)N(C5CCC(=O)NC5=O)C7=O)C6)CC4)c(OC)c3)cn(C)c(=O)c2C1] |
| F153 | 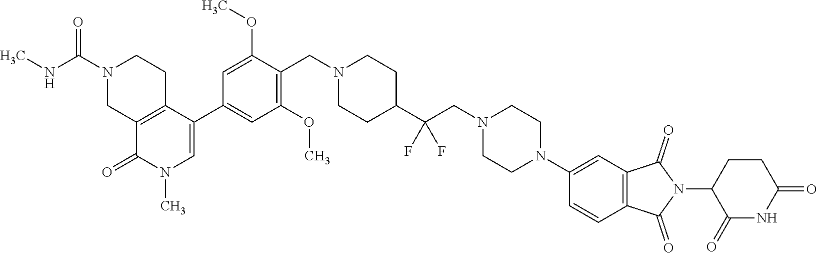 [SMILES: CNC(=O)N1CCc2c(-c3cc(OC)c(CN4CCC(C(F)(F)CN5CCN(c6ccc7c(c6)C(=O)N(C6CCC(=O)NC6=O)C7=O)CC5)CC4)c(OC)c3)cn(C)c(=O)c2C1] |
| F154 | 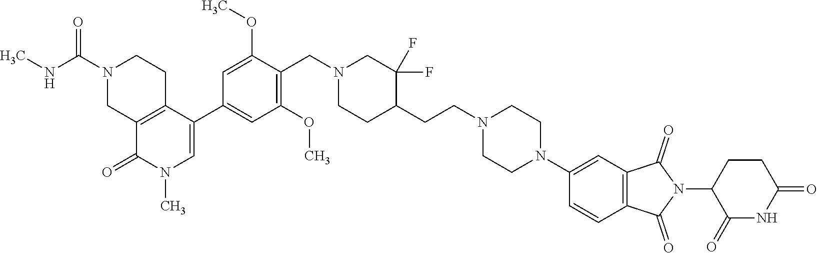 [SMILES: CNC(=O)N1CCc2c(-c3cc(OC)c(CN4CCC(CCN5CCN(c6ccc7c(c6)C(=O)N(C6CCC(=O)NC6=O)C7=O)CC5)C(F)(F)C4)c(OC)c3)cn(C)c(=O)c2C1] |
| F155 | 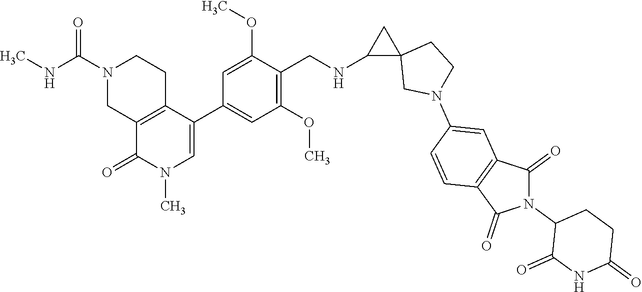 [SMILES: CNC(=O)N1CCc2c(-c3cc(OC)c(CNC4CC45CCN(c4ccc6c(c4)C(=O)N(C4CCC(=O)NC4=O)C6=O)C5)c(OC)c3)cn(C)c(=O)c2C1] |
| F156 | 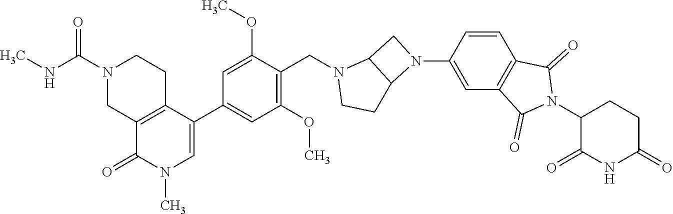 [SMILES: CNC(=O)N1CCc2c(-c3cc(OC)c(CN4CCC5C4CN5c4ccc5c(c4)C(=O)N(C4CCC(=O)NC4=O)C5=O)c(OC)c3)cn(C)c(=O)c2C1] |
| F157 | 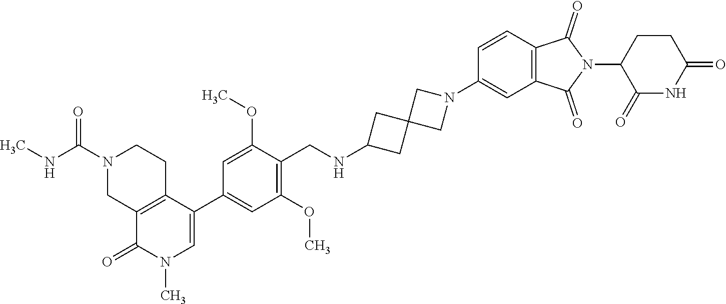 [SMILES: CNC(=O)N1CCc2c(-c3cc(OC)c(CNC4CC5(C4)CN(c4ccc6c(c4)C(=O)N(C4CCC(=O)NC4=O)C6=O)C5)c(OC)c3)cn(C)c(=O)c2C1] |
| F158 | 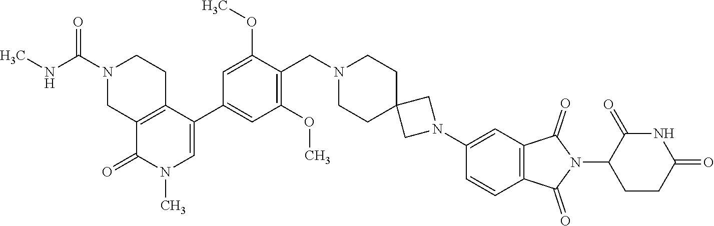 [SMILES: CNC(=O)N1CCc2c(-c3cc(OC)c(CN4CCC5(CC4)CN(c4ccc6c(c4)C(=O)N(C4CCC(=O)NC4=O)C6=O)C5)c(OC)c3)cn(C)c(=O)c2C1] |
| F159 | 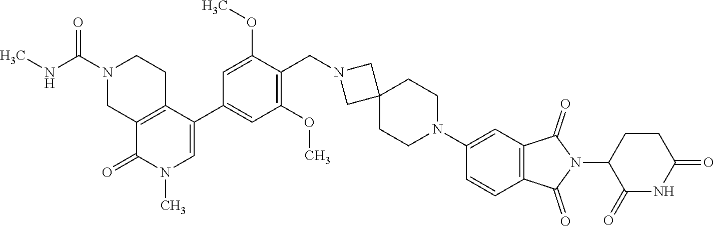 [SMILES: CNC(=O)N1CCc2c(-c3cc(OC)c(CN4CC5(CCN(c6ccc7c(c6)C(=O)N(C6CCC(=O)NC6=O)C7=O)CC5)C4)c(OC)c3)cn(C)c(=O)c2C1] |
| F160 | 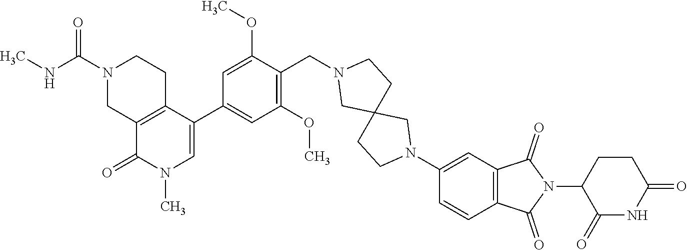 [SMILES: CNC(=O)N1CCc2c(-c3cc(OC)c(CN4CCC5(CCN(c6ccc7c(c6)C(=O)N(C6CCC(=O)NC6=O)C7=O)C5)C4)c(OC)c3)cn(C)c(=O)c2C1] |
| F161 | 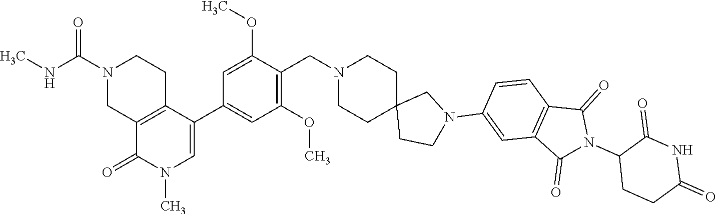 [SMILES: CNC(=O)N1CCc2c(-c3cc(OC)c(CN4CCC5(CC4)CCN(c4ccc6c(c4)C(=O)N(C4CCC(=O)NC4=O)C6=O)C5)c(OC)c3)cn(C)c(=O)c2C1] |
| F162 | 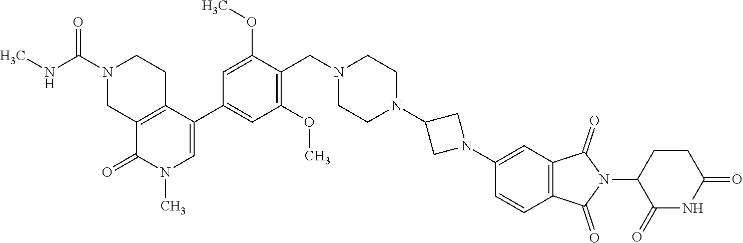 [SMILES: CNC(=O)N1CCc2c(-c3cc(OC)c(CN4CCN(C5CN(c6ccc7c(c6)C(=O)N(C6CCC(=O)NC6=O)C7=O)C5)CC4)c(OC)c3)cn(C)c(=O)c2C1] |
| F163 | 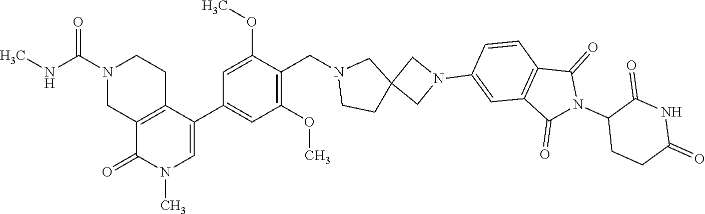 [SMILES: CNC(=O)N1CCc2c(-c3cc(OC)c(CN4CCC5(C4)CN(c4ccc6c(c4)C(=O)N(C4CCC(=O)NC4=O)C6=O)C5)c(OC)c3)cn(C)c(=O)c2C1] |
| F164 | 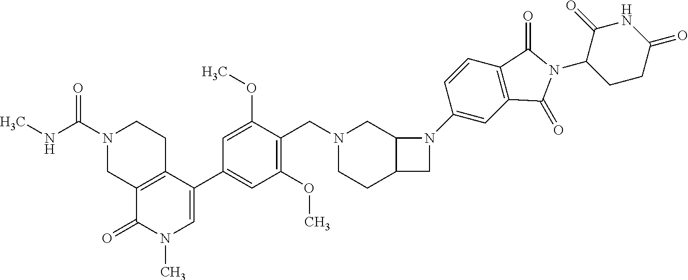 [SMILES: CNC(=O)N1CCc2c(-c3cc(OC)c(CN4CCC5CN(c6ccc7c(c6)C(=O)N(C6CCC(=O)NC6=O)C7=O)C5C4)c(OC)c3)cn(C)c(=O)c2C1] |
| F165 | 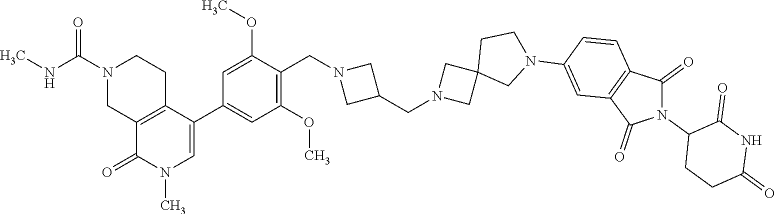 [SMILES: CNC(=O)N1CCc2c(-c3cc(OC)c(CN4CC(CN5CC6(CCN(c7ccc8c(c7)C(=O)N(C7CCC(=O)NC7=O)C8=O)C6)C5)C4)c(OC)c3)cn(C)c(=O)c2C1] |
| F166 | 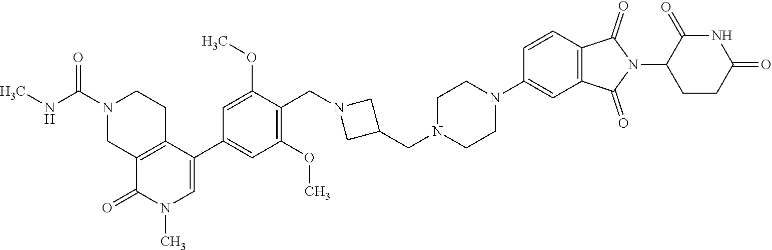 [SMILES: CNC(=O)N1CCc2c(-c3cc(OC)c(CN4CC(CN5CCN(c6ccc7c(c6)C(=O)N(C6CCC(=O)NC6=O)C7=O)CC5)C4)c(OC)c3)cn(C)c(=O)c2C1] |
| F167 | 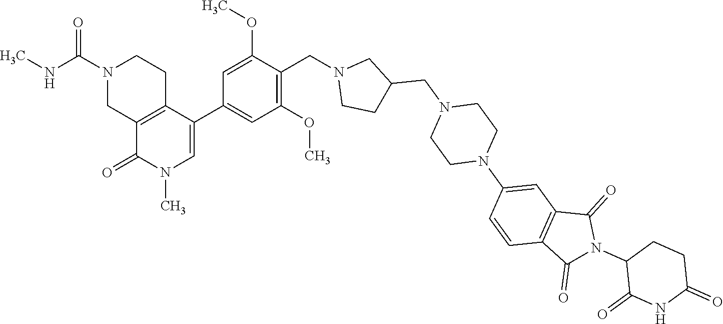 [SMILES: CNC(=O)N1CCc2c(-c3cc(OC)c(CN4CCC(CN5CCN(c6ccc7c(c6)C(=O)N(C6CCC(=O)NC6=O)C7=O)CC5)C4)c(OC)c3)cn(C)c(=O)c2C1] |
| F168 | 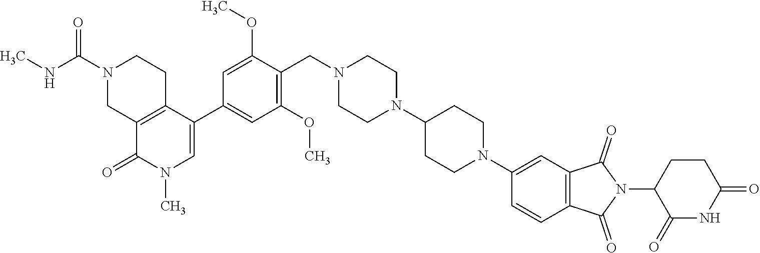 [SMILES: CNC(=O)N1CCc2c(-c3cc(OC)c(CN4CCN(C5CCN(c6ccc7c(c6)C(=O)N(C6CCC(=O)NC6=O)C7=O)CC5)CC4)c(OC)c3)cn(C)c(=O)c2C1] |
| F169 | 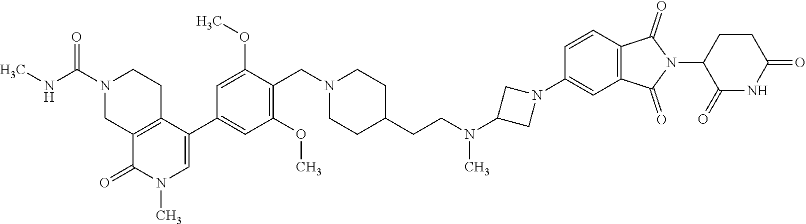 [SMILES: CNC(=O)N1CCc2c(-c3cc(OC)c(CN4CCC(CCN(C)C5CN(c6ccc7c(c6)C(=O)N(C6CCC(=O)NC6=O)C7=O)C5)CC4)c(OC)c3)cn(C)c(=O)c2C1] |
| F170 | 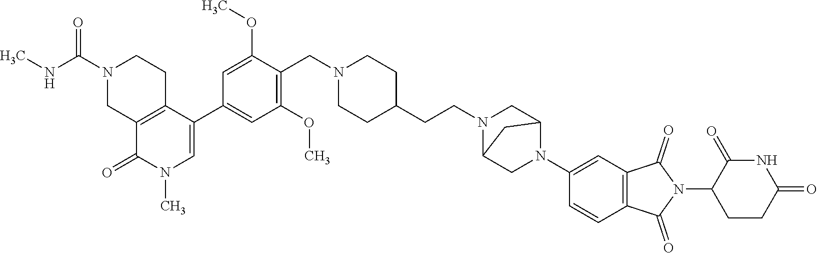 [SMILES: CNC(=O)N1CCc2c(-c3cc(OC)c(CN4CCC(CCN5CC6CC5CN6c5ccc6c(c5)C(=O)N(C5CCC(=O)NC5=O)C6=O)CC4)c(OC)c3)cn(C)c(=O)c2C1] |
| F171 | 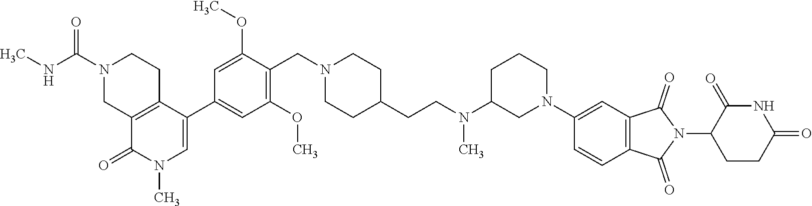 [SMILES: CNC(=O)N1CCc2c(-c3cc(OC)c(CN4CCC(CCN(C)C5CCCN(c6ccc7c(c6)C(=O)N(C6CCC(=O)NC6=O)C7=O)C5)CC4)c(OC)c3)cn(C)c(=O)c2C1] |
| F172 | 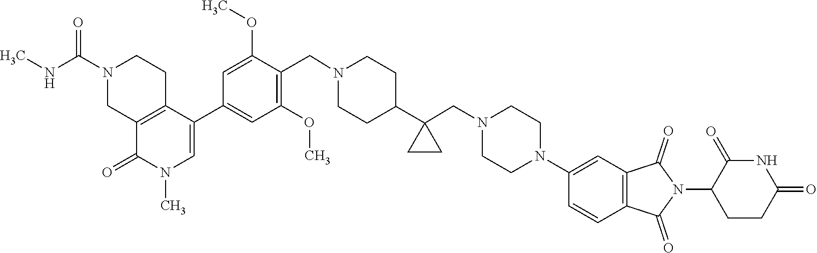 [SMILES: CNC(=O)N1CCc2c(-c3cc(OC)c(CN4CCC(C5(CN6CCN(c7ccc8c(c7)C(=O)N(C7CCC(=O)NC7=O)C8=O)CC6)CC5)CC4)c(OC)c3)cn(C)c(=O)c2C1] |
| F173 | 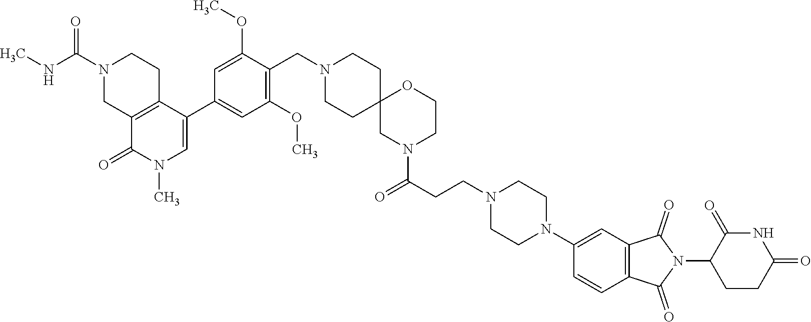 [SMILES: CNC(=O)N1CCc2c(-c3cc(OC)c(CN4CCC5(CC4)CN(C(=O)CCN4CCN(c6ccc7c(c6)C(=O)N(C6CCC(=O)NC6=O)C7=O)CC4)CCO5)c(OC)c3)cn(C)c(=O)c2C1] |
| F174 | 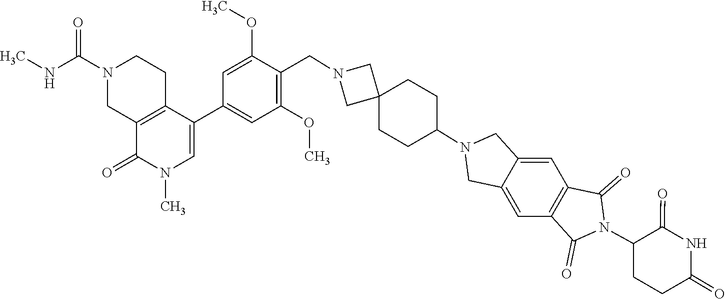 [SMILES: CNC(=O)N1CCc2c(-c3cc(OC)c(CN4CC5(CCC(N6Cc7cc8c(cc7C6)C(=O)N(C6CCC(=O)NC6=O)C8=O)CC5)C4)c(OC)c3)cn(C)c(=O)c2C1] |
| F175 | 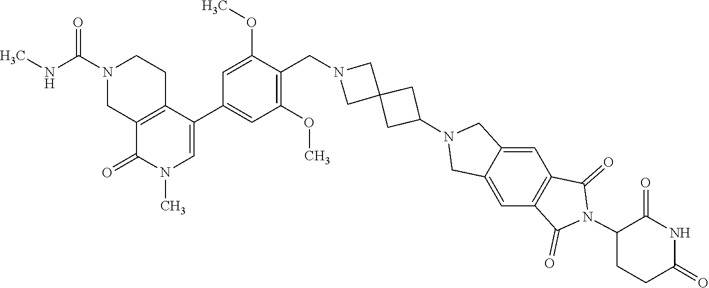 [SMILES: CNC(=O)N1CCc2c(-c3cc(OC)c(CN4CC5(CC(N6Cc7cc8c(cc7C6)C(=O)N(C6CCC(=O)NC6=O)C8=O)C5)C4)c(OC)c3)cn(C)c(=O)c2C1] |
| F176 | 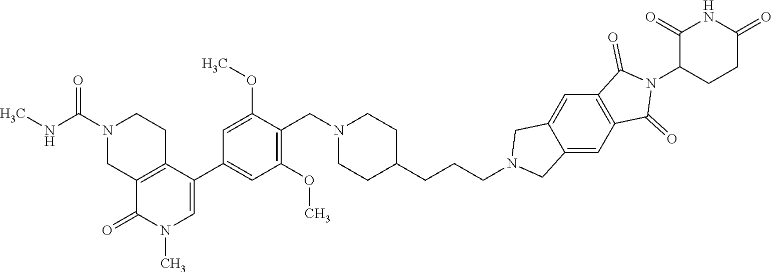 [SMILES: CNC(=O)N1CCc2c(-c3cc(OC)c(CN4CCC(CCCN5Cc6cc7c(cc6C5)C(=O)N(C5CCC(=O)NC5=O)C7=O)CC4)c(OC)c3)cn(C)c(=O)c2C1] |
| F177 | 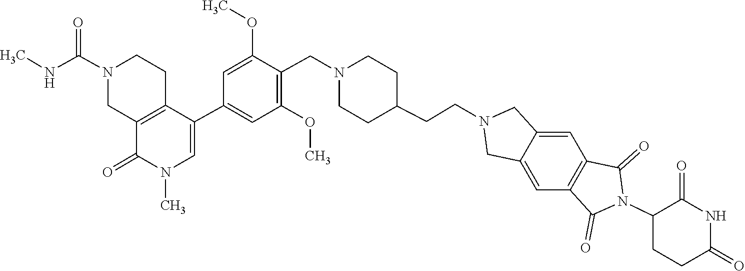 [SMILES: CNC(=O)N1CCc2c(-c3cc(OC)c(CN4CCC(CCN5Cc6cc7c(cc6C5)C(=O)N(C5CCC(=O)NC5=O)C7=O)CC4)c(OC)c3)cn(C)c(=O)c2C1] |
| F178 | 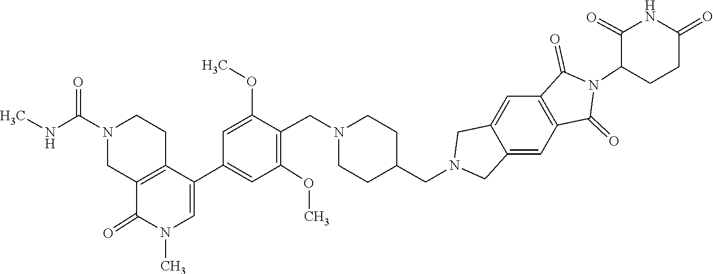 [SMILES: CNC(=O)N1CCc2c(-c3cc(OC)c(CN4CCC(CN5Cc6cc7c(cc6C5)C(=O)N(C5CCC(=O)NC5=O)C7=O)CC4)c(OC)c3)cn(C)c(=O)c2C1] |
| F179 | 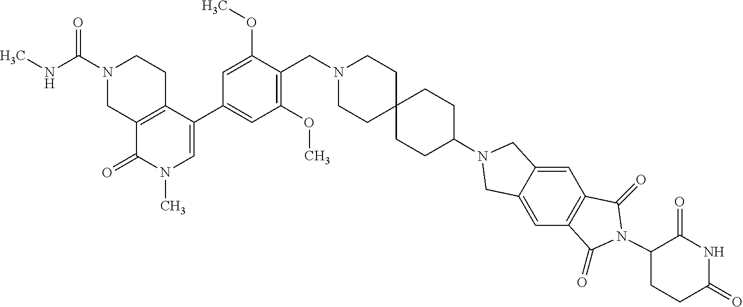 [SMILES: CNC(=O)N1CCc2c(-c3cc(OC)c(CN4CCC5(CCC(N6Cc7cc8c(cc7C6)C(=O)N(C6CCC(=O)NC6=O)C8=O)CC5)CC4)c(OC)c3)cn(C)c(=O)c2C1] |
| F180 | 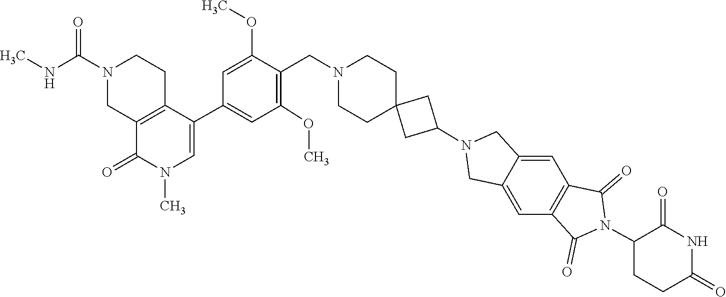 [SMILES: CNC(=O)N1CCc2c(-c3cc(OC)c(CN4CCC5(CC4)CC(N4Cc6cc7c(cc6C4)C(=O)N(C4CCC(=O)NC4=O)C7=O)C5)c(OC)c3)cn(C)c(=O)c2C1] |
| F181 | 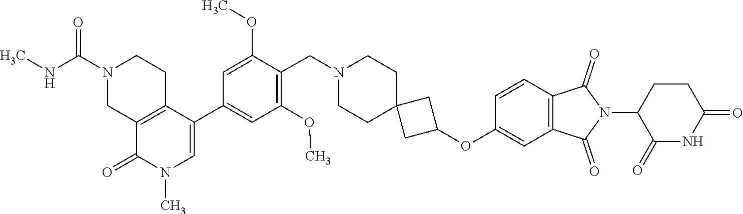 [SMILES: CNC(=O)N1CCc2c(-c3cc(OC)c(CN4CCC5(CC4)CC(Oc4ccc6c(c4)C(=O)N(C4CCC(=O)NC4=O)C6=O)C5)c(OC)c3)cn(C)c(=O)c2C1] |
| F182 | 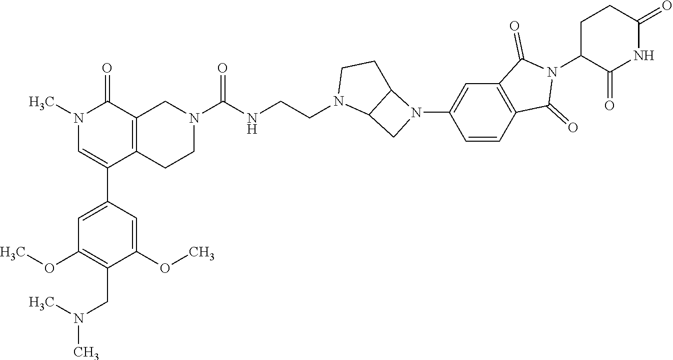 [SMILES: COc1cc(-c2cn(C)c(=O)c3c2CCN(C(=O)NCCN2CCC4C2CN4c2ccc4c(c2)C(=O)N(C2CCC(=O)NC2=O)C4=O)C3)cc(OC)c1CN(C)C] |
| F183 | 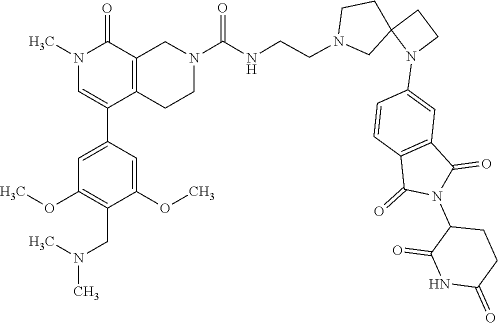 [SMILES: COc1cc(-c2cn(C)c(=O)c3c2CCN(C(=O)NCCN2CCC4(CCN4c4ccc5c(c4)C(=O)N(C4CCC(=O)NC4=O)C5=O)C2)C3)cc(OC)c1CN(C)C] |
| F184 | 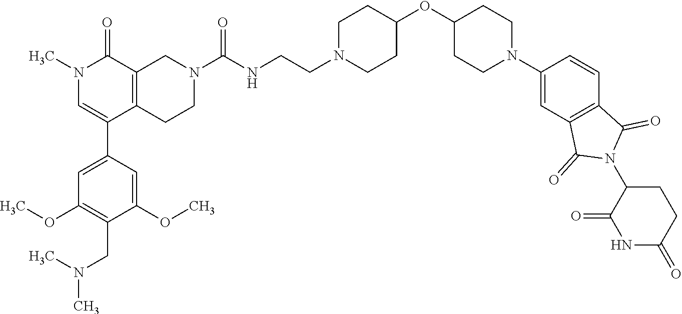 [SMILES: COc1cc(-c2cn(C)c(=O)c3c2CCN(C(=O)NCCN2CCC(OC4CCN(c5ccc6c(c5)C(=O)N(C5CCC(=O)NC5=O)C6=O)CC4)CC2)C3)cc(OC)c1CN(C)C] |
| F185 | 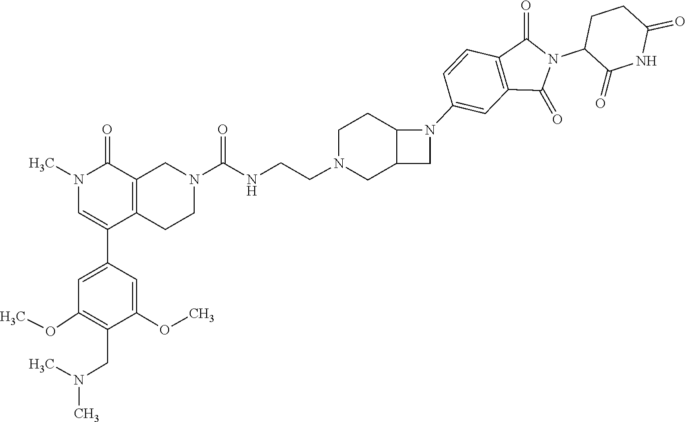 [SMILES: COc1cc(-c2cn(C)c(=O)c3c2CCN(C(=O)NCCN2CCC4C(C2)CN4c2ccc4c(c2)C(=O)N(C2CCC(=O)NC2=O)C4=O)C3)cc(OC)c1CN(C)C] |
| F186 | 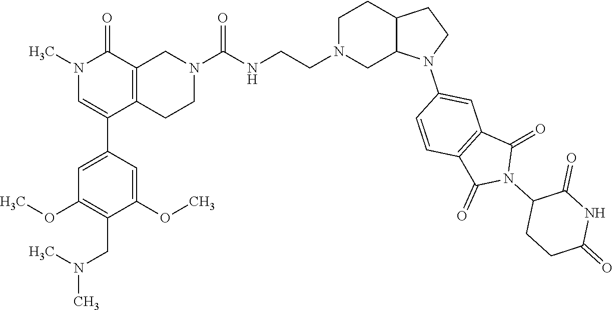 [SMILES: COc1cc(-c2cn(C)c(=O)c3c2CCN(C(=O)NCCN2CCC4CCN(c5ccc6c(c5)C(=O)N(C5CCC(=O)NC5=O)C6=O)C4C2)C3)cc(OC)c1CN(C)C] |
| F187 | 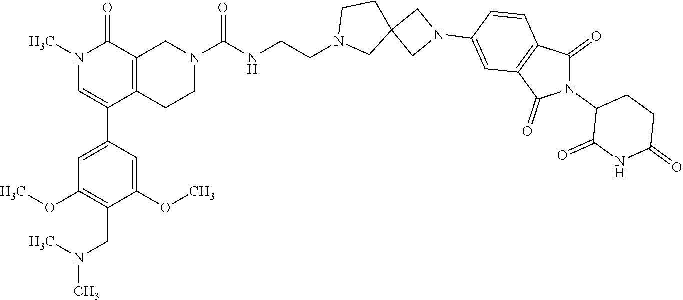 [SMILES: COc1cc(-c2cn(C)c(=O)c3c2CCN(C(=O)NCCN2CCC4(C2)CN(c2ccc5c(c2)C(=O)N(C2CCC(=O)NC2=O)C5=O)C4)C3)cc(OC)c1CN(C)C] |
| F188 | 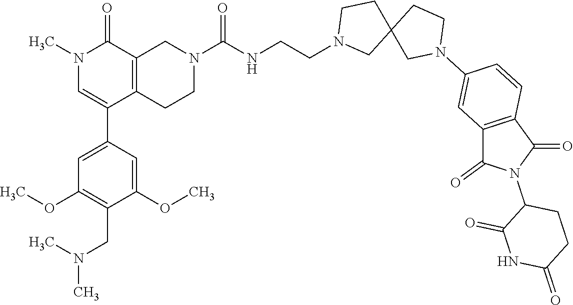 [SMILES: COc1cc(-c2cn(C)c(=O)c3c2CCN(C(=O)NCCN2CCC4(CCN(c5ccc6c(c5)C(=O)N(C5CCC(=O)NC5=O)C6=O)C4)C2)C3)cc(OC)c1CN(C)C] |
| F189 | 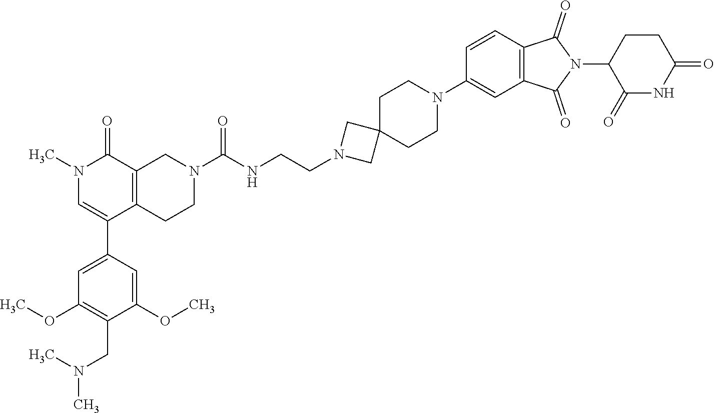 [SMILES: COc1cc(-c2cn(C)c(=O)c3c2CCN(C(=O)NCCN2CC4(CCN(c5ccc6c(c5)C(=O)N(C5CCC(=O)NC5=O)C6=O)CC4)C2)C3)cc(OC)c1CN(C)C] |
| F190 | 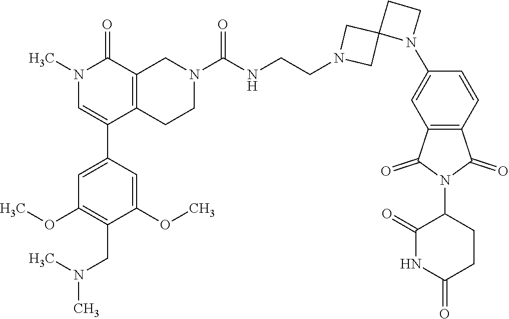 [SMILES: COc1cc(-c2cn(C)c(=O)c3c2CCN(C(=O)NCCN2CC4(CCN4c4ccc5c(c4)C(=O)N(C4CCC(=O)NC4=O)C5=O)C2)C3)cc(OC)c1CN(C)C] |
| F191 | 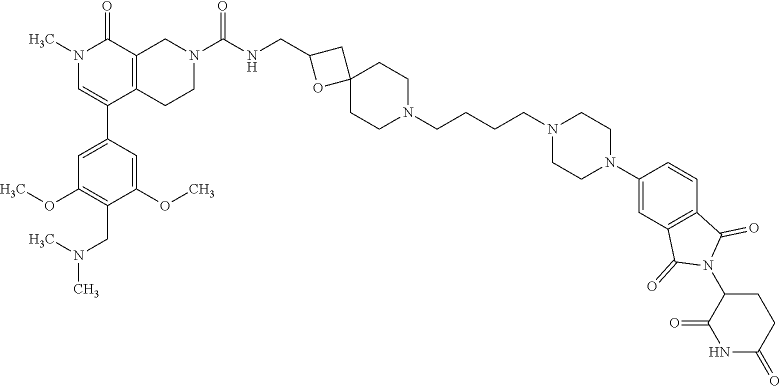 [SMILES: COc1cc(-c2cn(C)c(=O)c3c2CCN(C(=O)NCC2CC4(CCN(CCCCN5CCN(c6ccc7c(c6)C(=O)N(C6CCC(=O)NC6=O)C7=O)CC5)CC4)O2)C3)cc(OC)c1CN(C)C] |
| F192 | 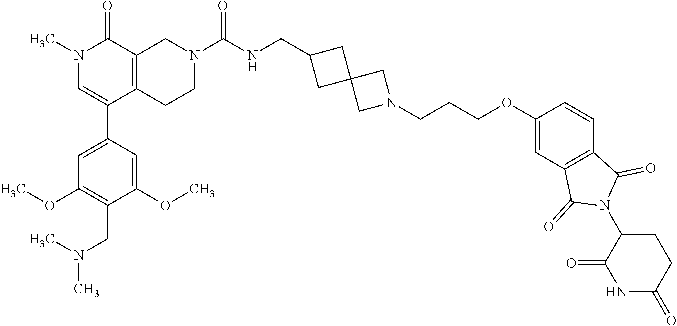 [SMILES: COc1cc(-c2cn(C)c(=O)c3c2CCN(C(=O)NCC2CC4(C2)CN(CCCOc2ccc5c(c2)C(=O)N(C2CCC(=O)NC2=O)C5=O)C4)C3)cc(OC)c1CN(C)C] |
| F193 | 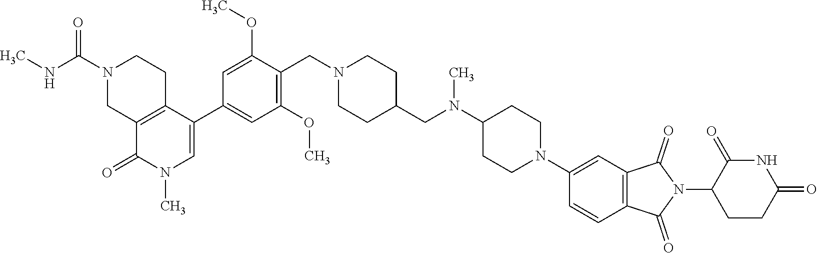 [SMILES: CNC(=O)N1CCc2c(-c3cc(OC)c(CN4CCC(CN(C)C5CCN(c6ccc7c(c6)C(=O)N(C6CCC(=O)NC6=O)C7=O)CC5)CC4)c(OC)c3)cn(C)c(=O)c2C1] |
| F194 | 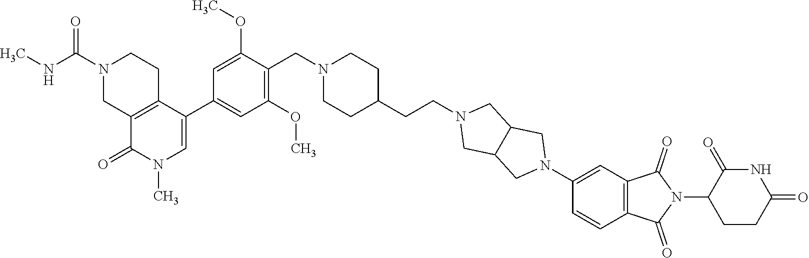 [SMILES: CNC(=O)N1CCc2c(-c3cc(OC)c(CN4CCC(CCN5CC6CN(c7ccc8c(c7)C(=O)N(C7CCC(=O)NC7=O)C8=O)CC6C5)CC4)c(OC)c3)cn(C)c(=O)c2C1] |
| F195 | 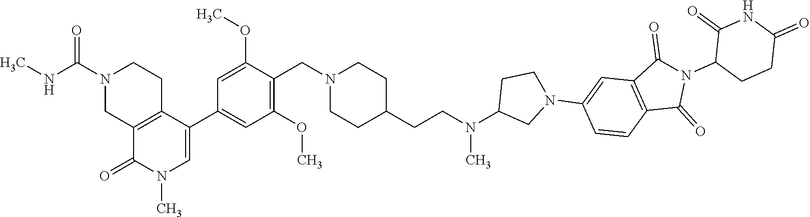 [SMILES: CNC(=O)N1CCc2c(-c3cc(OC)c(CN4CCC(CCN(C)C5CCN(c6ccc7c(c6)C(=O)N(C6CCC(=O)NC6=O)C7=O)C5)CC4)c(OC)c3)cn(C)c(=O)c2C1] |
| F196 | 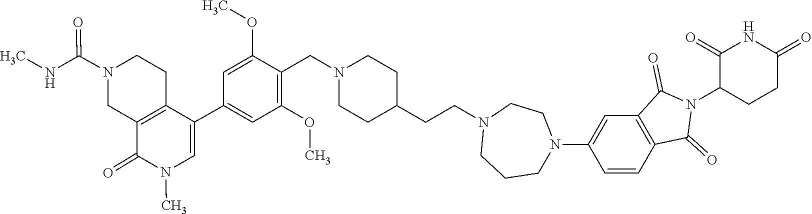 [SMILES: CNC(=O)N1CCc2c(-c3cc(OC)c(CN4CCC(CCN5CCCN(c6ccc7c(c6)C(=O)N(C6CCC(=O)NC6=O)C7=O)CC5)CC4)c(OC)c3)cn(C)c(=O)c2C1] |
| F197 | 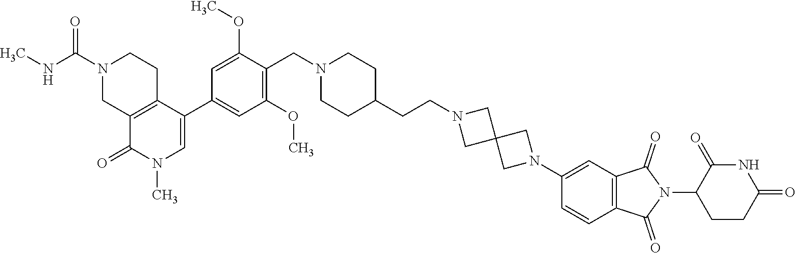 [SMILES: CNC(=O)N1CCc2c(-c3cc(OC)c(CN4CCC(CCN5CC6(C5)CN(c5ccc7c(c5)C(=O)N(C5CCC(=O)NC5=O)C7=O)C6)CC4)c(OC)c3)cn(C)c(=O)c2C1] |
| F198 | 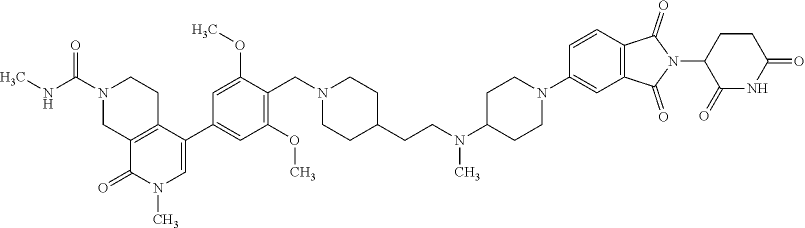 [SMILES: CNC(=O)N1CCc2c(-c3cc(OC)c(CN4CCC(CCN(C)C5CCN(c6ccc7c(c6)C(=O)N(C6CCC(=O)NC6=O)C7=O)CC5)CC4)c(OC)c3)cn(C)c(=O)c2C1] |
| F199 | 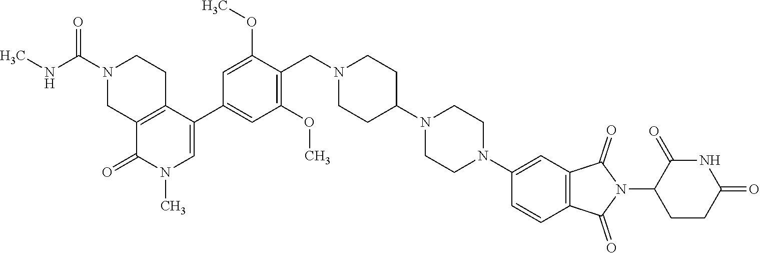 [SMILES: CNC(=O)N1CCc2c(-c3cc(OC)c(CN4CCC(N5CCN(c6ccc7c(c6)C(=O)N(C6CCC(=O)NC6=O)C7=O)CC5)CC4)c(OC)c3)cn(C)c(=O)c2C1] |
| F200 | 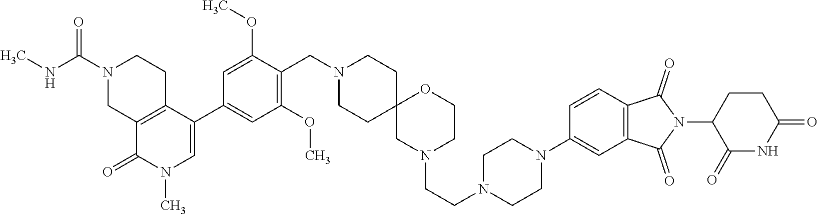 [SMILES: CNC(=O)N1CCc2c(-c3cc(OC)c(CN4CCC5(CC4)CN(CCN4CCN(c6ccc7c(c6)C(=O)N(C6CCC(=O)NC6=O)C7=O)CC4)CCO5)c(OC)c3)cn(C)c(=O)c2C1] |
| F201 | 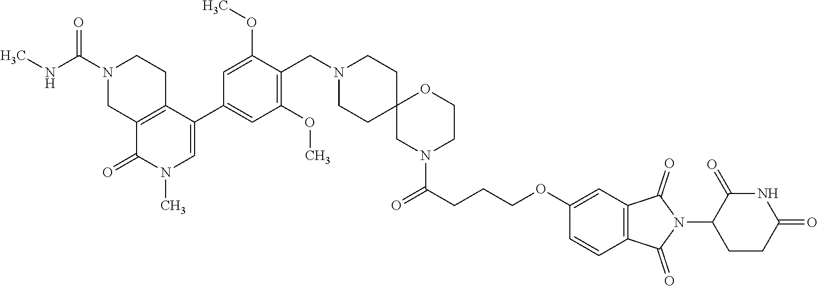 [SMILES: CNC(=O)N1CCc2c(-c3cc(OC)c(CN4CCC5(CC4)CN(C(=O)CCCOc4ccc6c(c4)C(=O)N(C4CCC(=O)NC4=O)C6=O)CCO5)c(OC)c3)cn(C)c(=O)c2C1] |
| F202 | 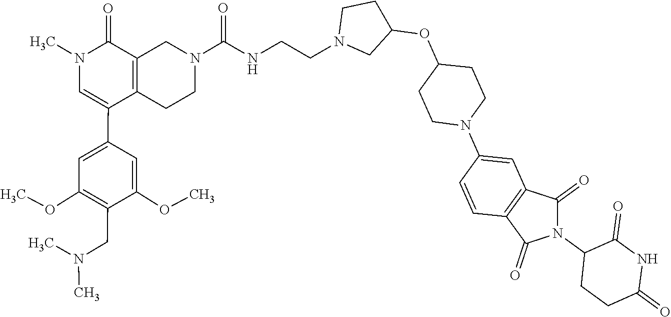 [SMILES: COc1cc(-c2cn(C)c(=O)c3c2CCN(C(=O)NCCN2CCC(OC4CCN(c5ccc6c(c5)C(=O)N(C5CCC(=O)NC5=O)C6=O)CC4)C2)C3)cc(OC)c1CN(C)C] |
| F203 | 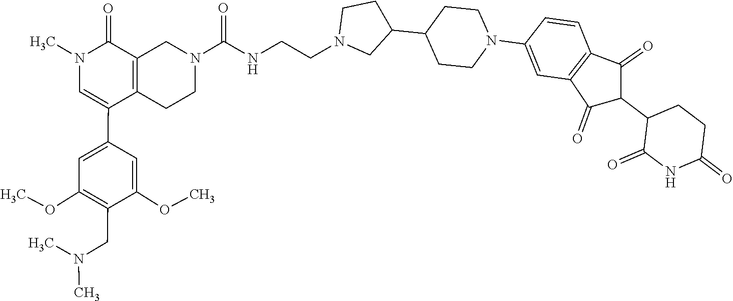 [SMILES: COc1cc(-c2cn(C)c(=O)c3c2CCN(C(=O)NCCN2CCC(C4CCN(c5ccc6c(c5)C(=O)C(C5CCC(=O)NC5=O)C6=O)CC4)C2)C3)cc(OC)c1CN(C)C] |
| F204 | 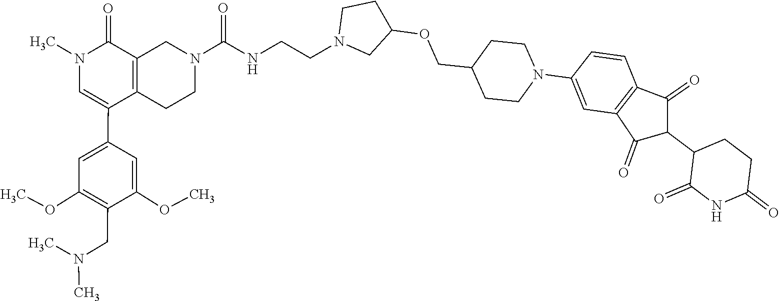 [SMILES: COc1cc(-c2cn(C)c(=O)c3c2CCN(C(=O)NCCN2CCC(OCC4CCN(c5ccc6c(c5)C(=O)C(C5CCC(=O)NC5=O)C6=O)CC4)C2)C3)cc(OC)c1CN(C)C] |
| F205 | 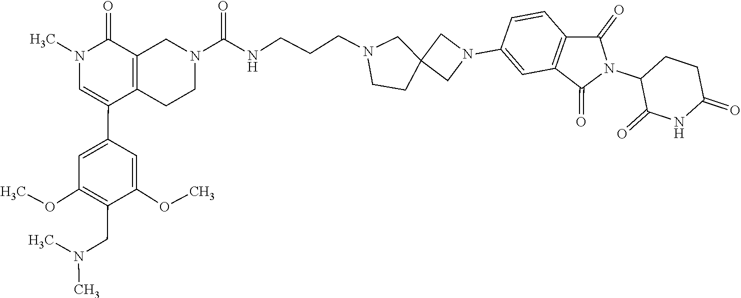 [SMILES: COc1cc(-c2cn(C)c(=O)c3c2CCN(C(=O)NCCCN2CCC4(C2)CN(c2ccc5c(c2)C(=O)N(C2CCC(=O)NC2=O)C5=O)C4)C3)cc(OC)c1CN(C)C] |
| F206 | 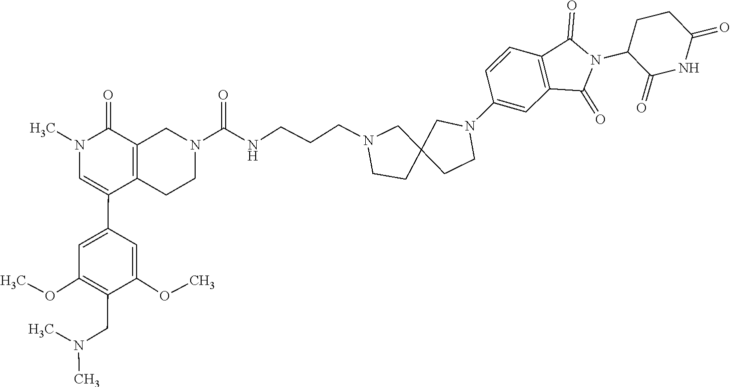 [SMILES: COc1cc(-c2cn(C)c(=O)c3c2CCN(C(=O)NCCCN2CCC4(CCN(c5ccc6c(c5)C(=O)N(C5CCC(=O)NC5=O)C6=O)C4)C2)C3)cc(OC)c1CN(C)C] |
| F207 | 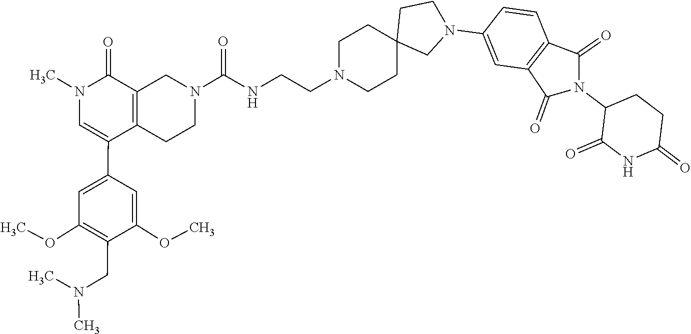 [SMILES: COc1cc(-c2cn(C)c(=O)c3c2CCN(C(=O)NCCN2CCC4(CC2)CCN(c2ccc5c(c2)C(=O)N(C2CCC(=O)NC2=O)C5=O)C4)C3)cc(OC)c1CN(C)C] |
| F208 | 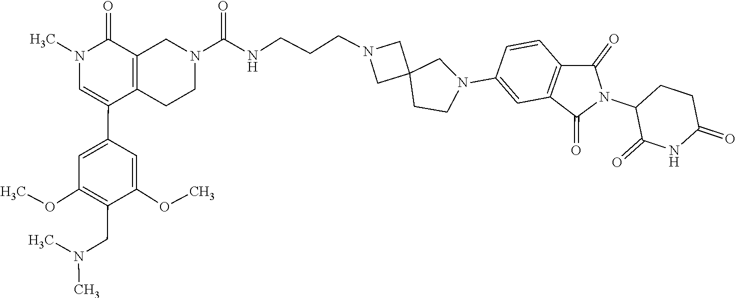 [SMILES: COc1cc(-c2cn(C)c(=O)c3c2CCN(C(=O)NCCCN2CC4(CCN(c5ccc6c(c5)C(=O)N(C5CCC(=O)NC5=O)C6=O)C4)C2)C3)cc(OC)c1CN(C)C] |
| F209 | 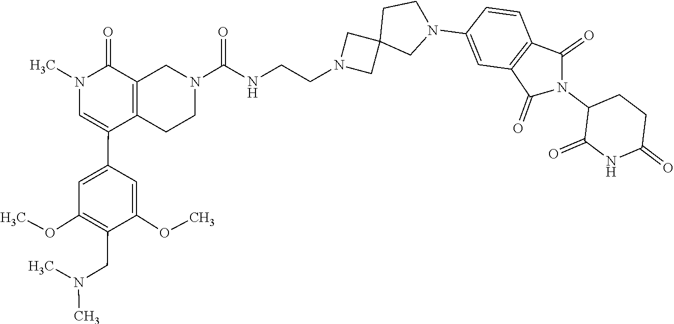 [SMILES: COc1cc(-c2cn(C)c(=O)c3c2CCN(C(=O)NCCN2CC4(CCN(c5ccc6c(c5)C(=O)N(C5CCC(=O)NC5=O)C6=O)C4)C2)C3)cc(OC)c1CN(C)C] |
| F210 | 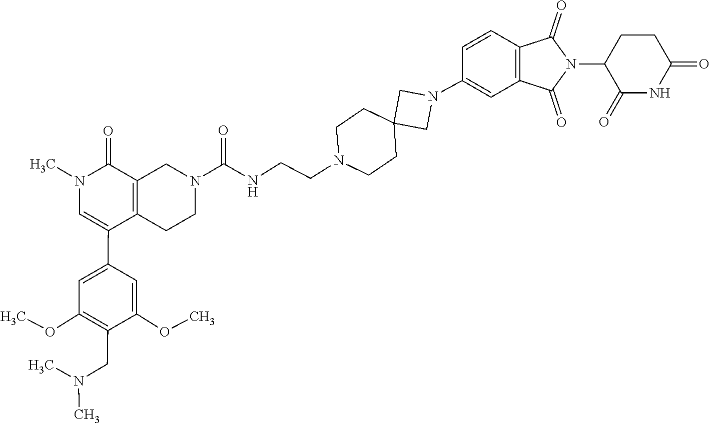 [SMILES: COc1cc(-c2cn(C)c(=O)c3c2CCN(C(=O)NCCN2CCC4(CC2)CN(c2ccc5c(c2)C(=O)N(C2CCC(=O)NC2=O)C5=O)C4)C3)cc(OC)c1CN(C)C] |
| F211 | 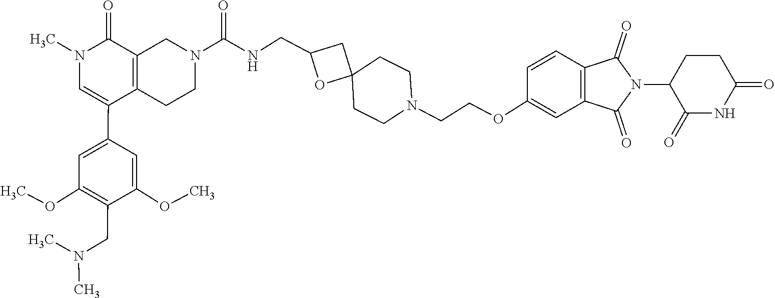 [SMILES: COc1cc(-c2cn(C)c(=O)c3c2CCN(C(=O)NCC2CC4(CCN(CCOc5ccc6c(c5)C(=O)N(C5CCC(=O)NC5=O)C6=O)CC4)O2)C3)cc(OC)c1CN(C)C] |
| F212 | 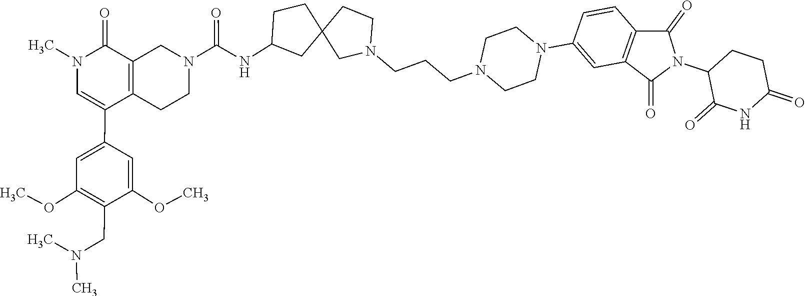 [SMILES: COc1cc(-c2cn(C)c(=O)c3c2CCN(C(=O)NC2CCC4(CCN(CCCN5CCN(c6ccc7c(c6)C(=O)N(C6CCC(=O)NC6=O)C7=O)CC5)C4)C2)C3)cc(OC)c1CN(C)C] |
| F213 | 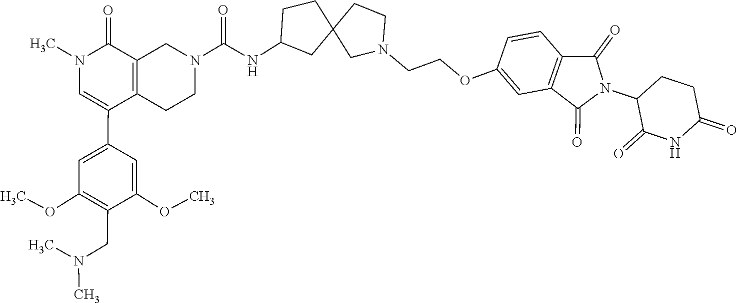 [SMILES: COc1cc(-c2cn(C)c(=O)c3c2CCN(C(=O)NC2CCC4(CCN(CCOc5ccc6c(c5)C(=O)N(C5CCC(=O)NC5=O)C6=O)C4)C2)C3)cc(OC)c1CN(C)C] |
| F214 | 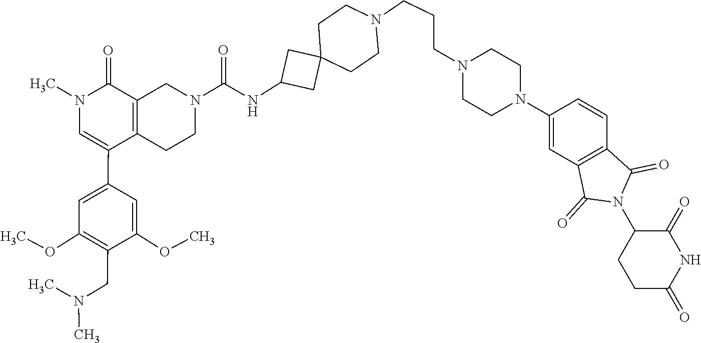 [SMILES: COc1cc(-c2cn(C)c(=O)c3c2CCN(C(=O)NC2CC4(CCN(CCCN5CCN(c6ccc7c(c6)C(=O)N(C6CCC(=O)NC6=O)C7=O)CC5)CC4)C2)C3)cc(OC)c1CN(C)C] |
| F215 | 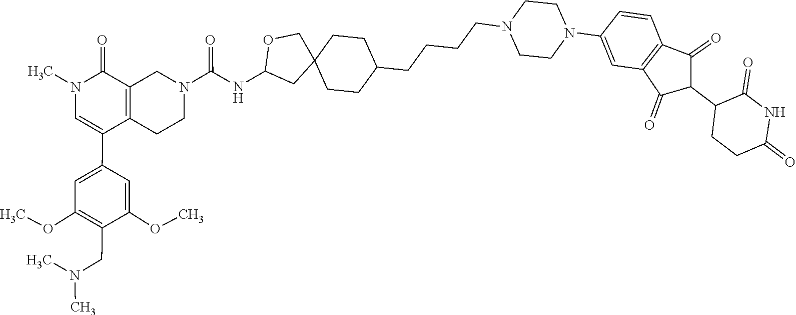 [SMILES: COc1cc(-c2cn(C)c(=O)c3c2CCN(C(=O)NC2CC4(CCC(CCCCN5CCN(c6ccc7c(c6)C(=O)C(C6CCC(=O)NC6=O)C7=O)CC5)CC4)CO2)C3)cc(OC)c1CN(C)C] |
| F216 | 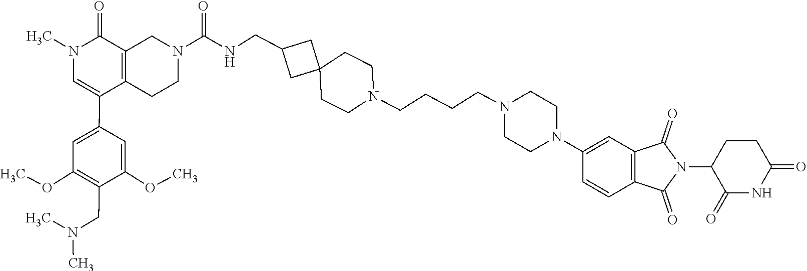 [SMILES: COc1cc(-c2cn(C)c(=O)c3c2CCN(C(=O)NCC2CC4(CCN(CCCCN5CCN(c6ccc7c(c6)C(=O)N(C6CCC(=O)NC6=O)C7=O)CC5)CC4)C2)C3)cc(OC)c1CN(C)C] |
| F217 | 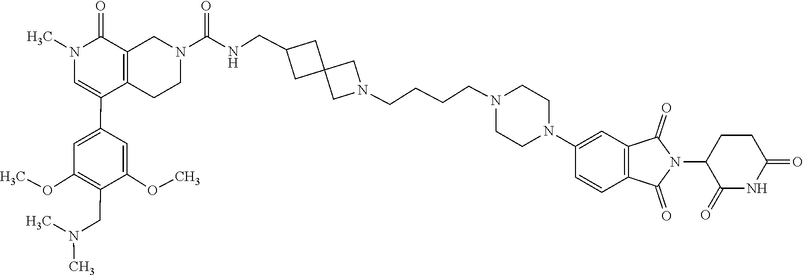 [SMILES: COc1cc(-c2cn(C)c(=O)c3c2CCN(C(=O)NCC2CC4(C2)CN(CCCCN2CCN(c5ccc6c(c5)C(=O)N(C5CCC(=O)NC5=O)C6=O)CC2)C4)C3)cc(OC)c1CN(C)C] |
| F218 | 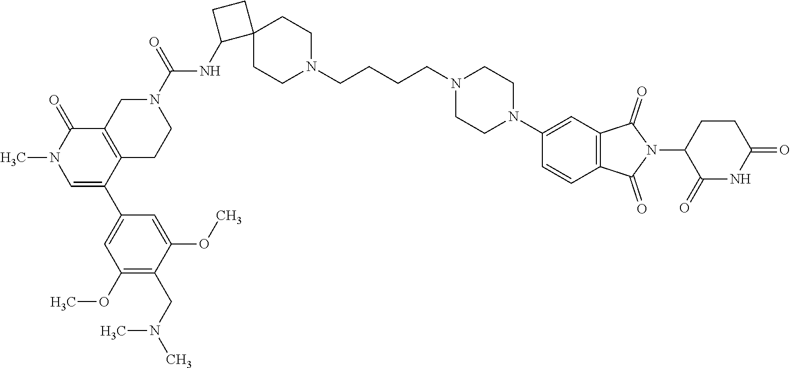 [SMILES: COc1cc(-c2cn(C)c(=O)c3c2CCN(C(=O)NC2CCC24CCN(CCCCN2CCN(c5ccc6c(c5)C(=O)N(C5CCC(=O)NC5=O)C6=O)CC2)CC4)C3)cc(OC)c1CN(C)C] |
| F219 | 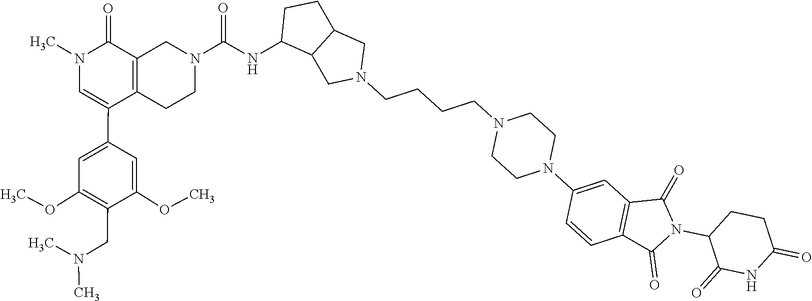 [SMILES: COc1cc(-c2cn(C)c(=O)c3c2CCN(C(=O)NC2CCC4CN(CCCCN5CCN(c6ccc7c(c6)C(=O)N(C6CCC(=O)NC6=O)C7=O)CC5)CC42)C3)cc(OC)c1CN(C)C] |
| F220 | 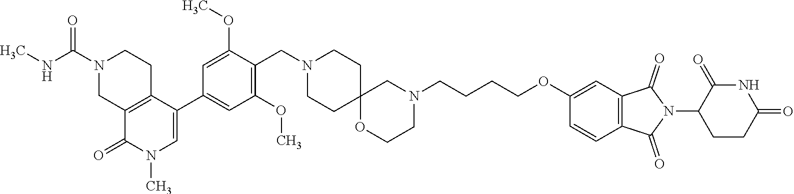 [SMILES: CNC(=O)N1CCc2c(-c3cc(OC)c(CN4CCC5(CC4)CN(CCCCOc4ccc6c(c4)C(=O)N(C4CCC(=O)NC4=O)C6=O)CCO5)c(OC)c3)cn(C)c(=O)c2C1] |
| F221 | 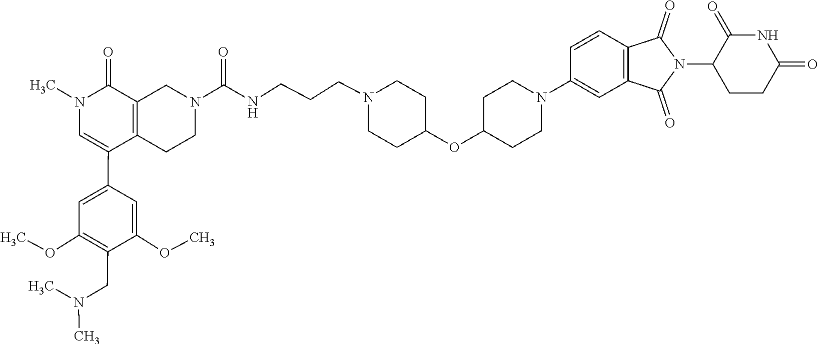 [SMILES: COc1cc(-c2cn(C)c(=O)c3c2CCN(C(=O)NCCCN2CCC(OC4CCN(c5ccc6c(c5)C(=O)N(C5CCC(=O)NC5=O)C6=O)CC4)CC2)C3)cc(OC)c1CN(C)C] |
| F222 | 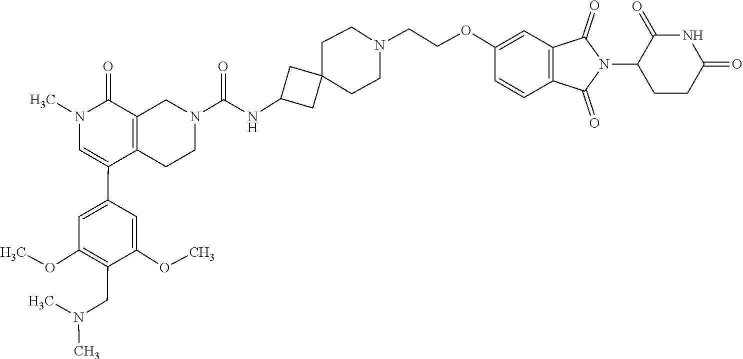 [SMILES: COc1cc(-c2cn(C)c(=O)c3c2CCN(C(=O)NC2CC4(CCN(CCOc5ccc6c(c5)C(=O)N(C5CCC(=O)NC5=O)C6=O)CC4)C2)C3)cc(OC)c1CN(C)C] |
| F223 | 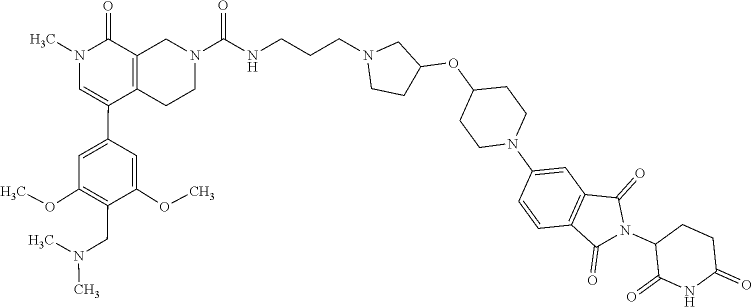 [SMILES: COc1cc(-c2cn(C)c(=O)c3c2CCN(C(=O)NCCCN2CCC(OC4CCN(c5ccc6c(c5)C(=O)N(C5CCC(=O)NC5=O)C6=O)CC4)C2)C3)cc(OC)c1CN(C)C] |
| F224 | 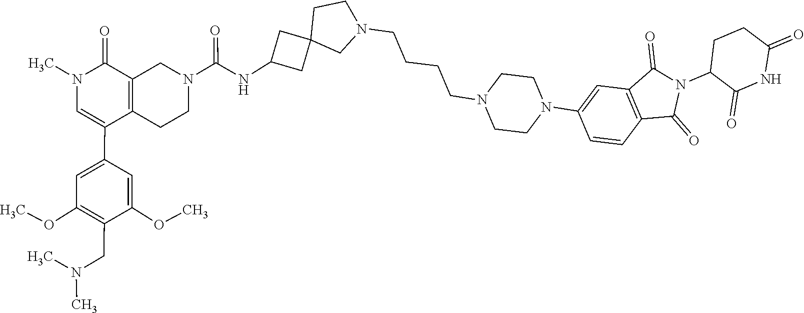 [SMILES: COc1cc(-c2cn(C)c(=O)c3c2CCN(C(=O)NC2CC4(CCN(CCCCN5CCN(c6ccc7c(c6)C(=O)N(C6CCC(=O)NC6=O)C7=O)CC5)C4)C2)C3)cc(OC)c1CN(C)C] |
| F225 | 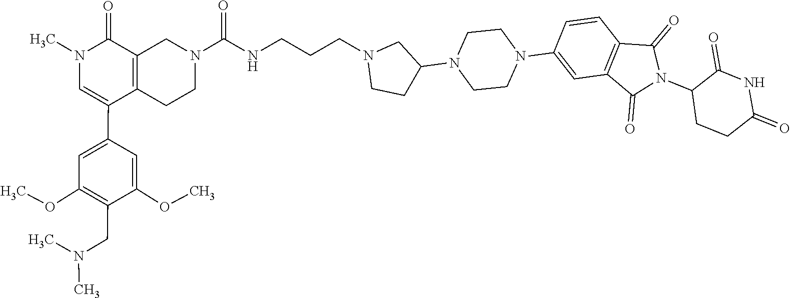 [SMILES: COc1cc(-c2cn(C)c(=O)c3c2CCN(C(=O)NCCCN2CCC(N4CCN(c5ccc6c(c5)C(=O)N(C5CCC(=O)NC5=O)C6=O)CC4)C2)C3)cc(OC)c1CN(C)C] |
| F226 | 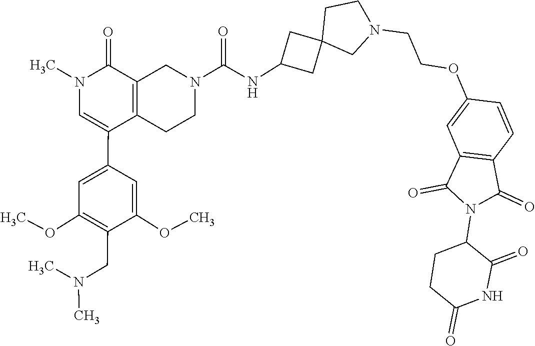 [SMILES: COc1cc(-c2cn(C)c(=O)c3c2CCN(C(=O)NC2CC4(CCN(CCOc5ccc6c(c5)C(=O)N(C5CCC(=O)NC5=O)C6=O)C4)C2)C3)cc(OC)c1CN(C)C] |
| F227 | 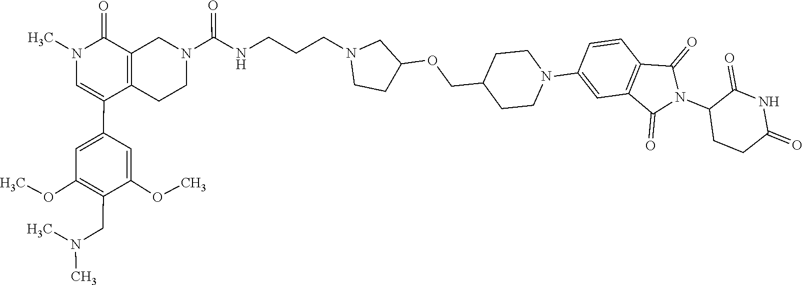 [SMILES: COc1cc(-c2cn(C)c(=O)c3c2CCN(C(=O)NCCCN2CCC(OCC4CCN(c5ccc6c(c5)C(=O)N(C5CCC(=O)NC5=O)C6=O)CC4)C2)C3)cc(OC)c1CN(C)C] |
| F228 | 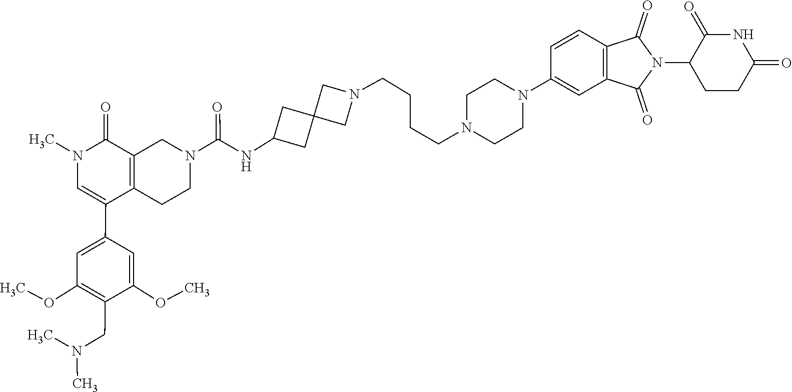 [SMILES: COc1cc(-c2cn(C)c(=O)c3c2CCN(C(=O)NC2CC4(C2)CN(CCCCN2CCN(c5ccc6c(c5)C(=O)N(C5CCC(=O)NC5=O)C6=O)CC2)C4)C3)cc(OC)c1CN(C)C] |
| F229 | 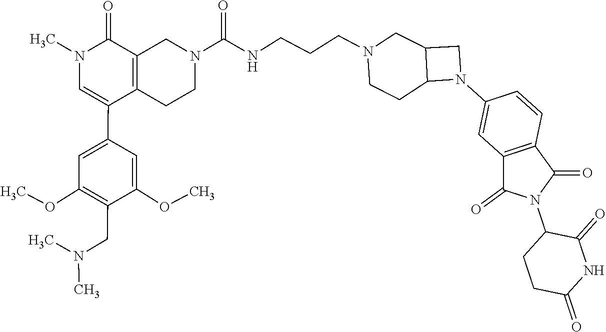 [SMILES: COc1cc(-c2cn(C)c(=O)c3c2CCN(C(=O)NCCCN2CCC4C(C2)CN4c2ccc4c(c2)C(=O)N(C2CCC(=O)NC2=O)C4=O)C3)cc(OC)c1CN(C)C] |
| F230 | 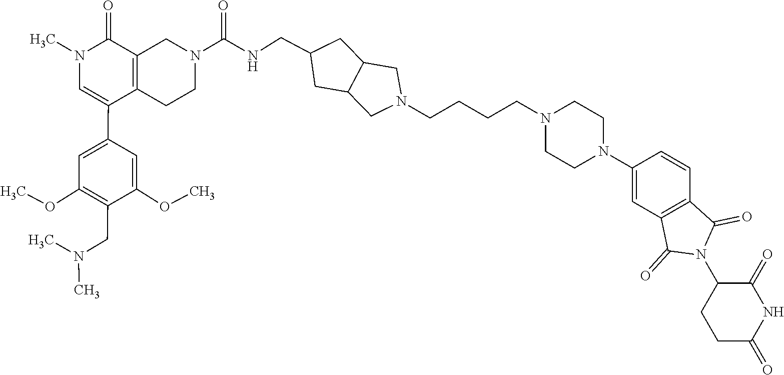 [SMILES: COc1cc(-c2cn(C)c(=O)c3c2CCN(C(=O)NCC2CC4CN(CCCCN5CCN(c6ccc7c(c6)C(=O)N(C6CCC(=O)NC6=O)C7=O)CC5)CC4C2)C3)cc(OC)c1CN(C)C] |
| F231 | 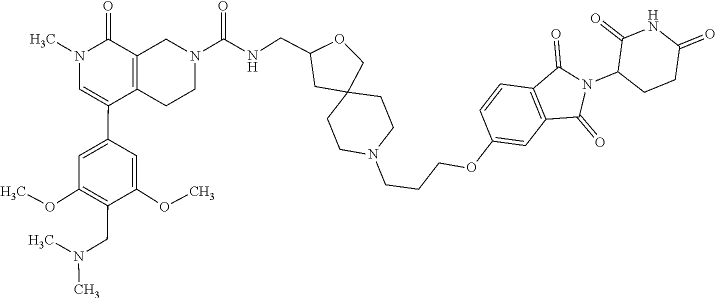 [SMILES: COc1cc(-c2cn(C)c(=O)c3c2CCN(C(=O)NCC2CC4(CCN(CCCOc5ccc6c(c5)C(=O)N(C5CCC(=O)NC5=O)C6=O)CC4)CO2)C3)cc(OC)c1CN(C)C] |
| F232 | 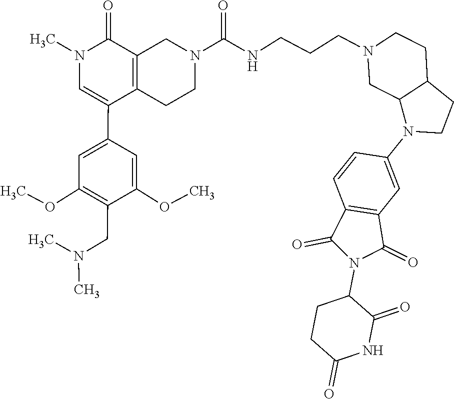 [SMILES: COc1cc(-c2cn(C)c(=O)c3c2CCN(C(=O)NCCCN2CCC4CCN(c5ccc6c(c5)C(=O)N(C5CCC(=O)NC5=O)C6=O)C4C2)C3)cc(OC)c1CN(C)C] |
| F233 | 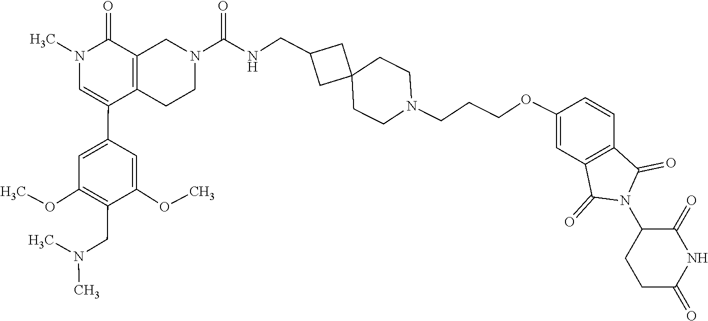 [SMILES: COc1cc(-c2cn(C)c(=O)c3c2CCN(C(=O)NCC2CC4(CCN(CCCOc5ccc6c(c5)C(=O)N(C5CCC(=O)NC5=O)C6=O)CC4)C2)C3)cc(OC)c1CN(C)C] |
| F234 | 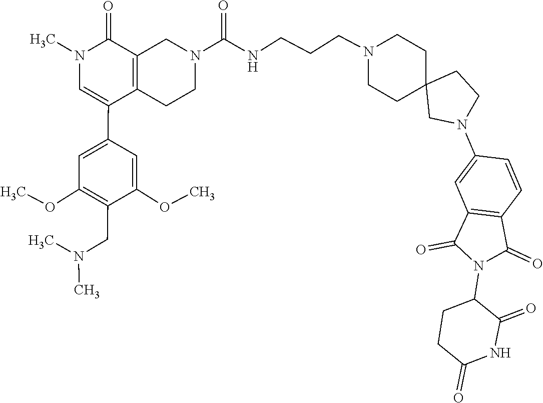 [SMILES: COc1cc(-c2cn(C)c(=O)c3c2CCN(C(=O)NCCCN2CCC4(CC2)CCN(c2ccc5c(c2)C(=O)N(C2CCC(=O)NC2=O)C5=O)C4)C3)cc(OC)c1CN(C)C] |
| F235 | 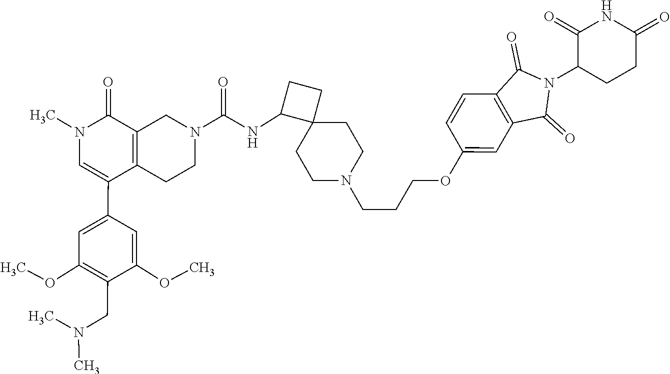 [SMILES: COc1cc(-c2cn(C)c(=O)c3c2CCN(C(=O)NC2CCC24CCN(CCCOc2ccc5c(c2)C(=O)N(C2CCC(=O)NC2=O)C5=O)CC4)C3)cc(OC)c1CN(C)C] |
| F236 | 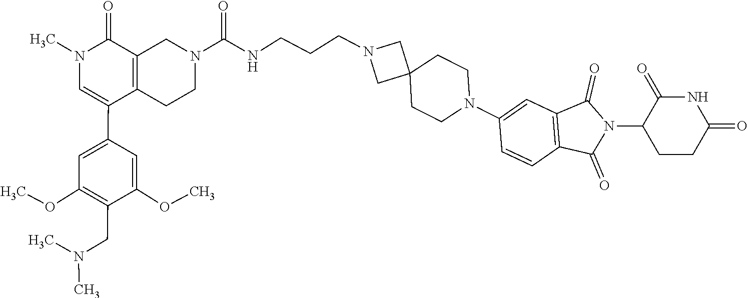 [SMILES: COc1cc(-c2cn(C)c(=O)c3c2CCN(C(=O)NCCCN2CC4(CCN(c5ccc6c(c5)C(=O)N(C5CCC(=O)NC5=O)C6=O)CC4)C2)C3)cc(OC)c1CN(C)C] |
| F237 | 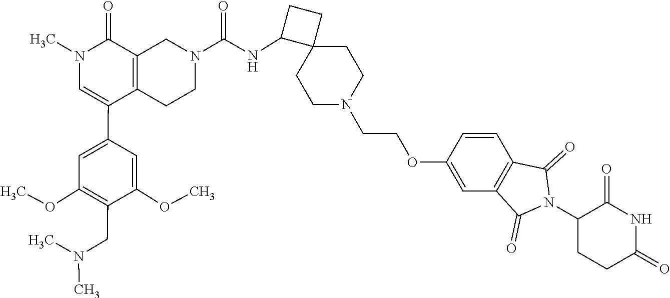 [SMILES: COc1cc(-c2cn(C)c(=O)c3c2CCN(C(=O)NC2CCC24CCN(CCOc2ccc5c(c2)C(=O)N(C2CCC(=O)NC2=O)C5=O)CC4)C3)cc(OC)c1CN(C)C] |
| F238 | 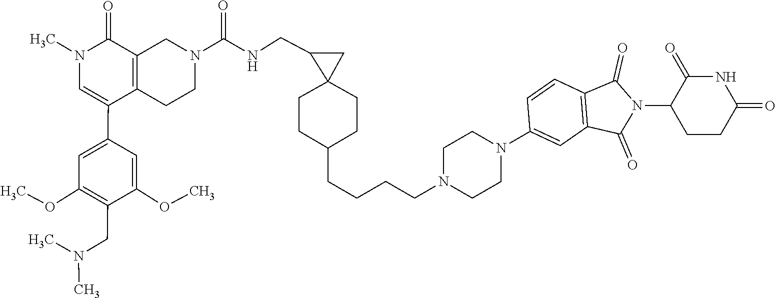 [SMILES: COc1cc(-c2cn(C)c(=O)c3c2CCN(C(=O)NCC2CC24CCC(CCCCN2CCN(c5ccc6c(c5)C(=O)N(C5CCC(=O)NC5=O)C6=O)CC2)CC4)C3)cc(OC)c1CN(C)C] |
| F239 | 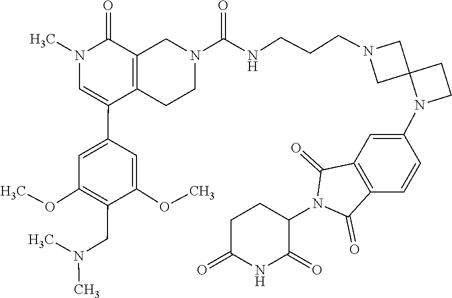 [SMILES: COc1cc(-c2cn(C)c(=O)c3c2CCN(C(=O)NCCCN2CC4(CCN4c4ccc5c(c4)C(=O)N(C4CCC(=O)NC4=O)C5=O)C2)C3)cc(OC)c1CN(C)C] |
| F240 | 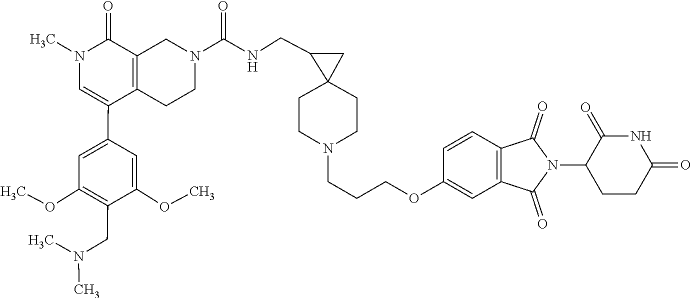 [SMILES: COc1cc(-c2cn(C)c(=O)c3c2CCN(C(=O)NCC2CC24CCN(CCCOc2ccc5c(c2)C(=O)N(C2CCC(=O)NC2=O)C5=O)CC4)C3)cc(OC)c1CN(C)C] |
| F241 | 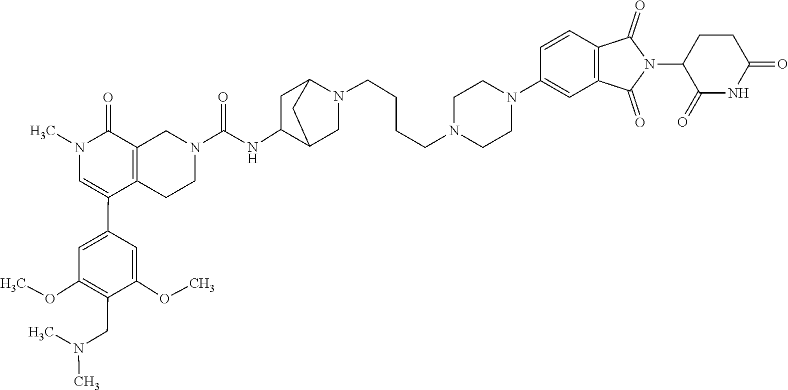 [SMILES: COc1cc(-c2cn(C)c(=O)c3c2CCN(C(=O)NC2CC4CC2CN4CCCCN2CCN(c4ccc5c(c4)C(=O)N(C4CCC(=O)NC4=O)C5=O)CC2)C3)cc(OC)c1CN(C)C] |
| F242 | 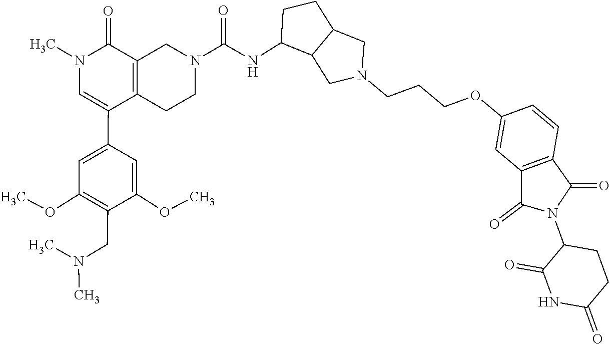 [SMILES: COc1cc(-c2cn(C)c(=O)c3c2CCN(C(=O)NC2CCC4CN(CCCOc5ccc6c(c5)C(=O)N(C5CCC(=O)NC5=O)C6=O)CC42)C3)cc(OC)c1CN(C)C] |
| F243 | 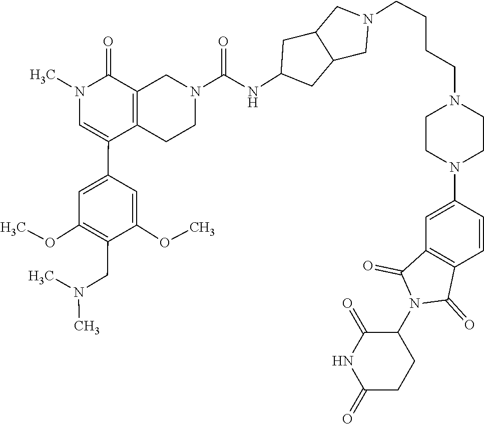 [SMILES: COc1cc(-c2cn(C)c(=O)c3c2CCN(C(=O)NC2CC4CN(CCCCN5CCN(c6ccc7c(c6)C(=O)N(C6CCC(=O)NC6=O)C7=O)CC5)CC4C2)C3)cc(OC)c1CN(C)C] |
| F244 | 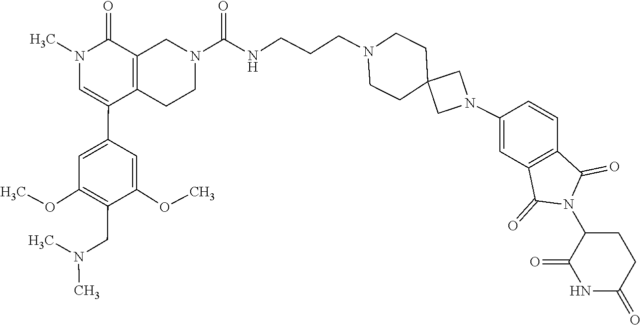 [SMILES: COc1cc(-c2cn(C)c(=O)c3c2CCN(C(=O)NCCCN2CCC4(CC2)CN(c2ccc5c(c2)C(=O)N(C2CCC(=O)NC2=O)C5=O)C4)C3)cc(OC)c1CN(C)C] |
| F245 | 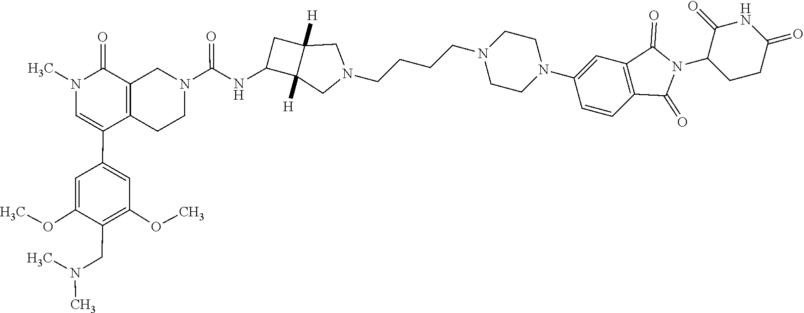 [SMILES: COc1cc(-c2cn(C)c(=O)c3c2CCN(C(=O)NC2C[C@@H]4CN(CCCCN5CCN(c6ccc7c(c6)C(=O)N(C6CCC(=O)NC6=O)C7=O)CC5)C[C@H]24)C3)cc(OC)c1CN(C)C] |
| F246 | 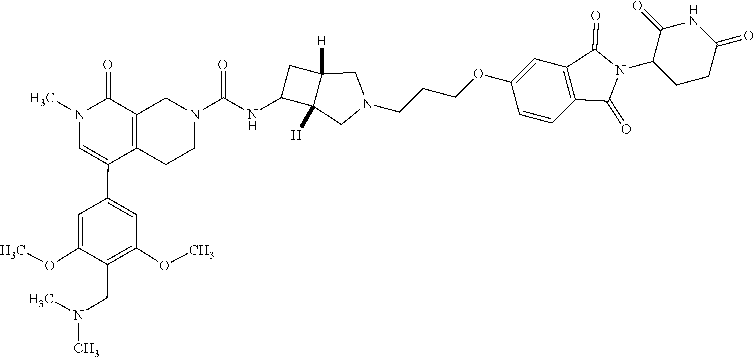 [SMILES: COc1cc(-c2cn(C)c(=O)c3c2CCN(C(=O)NC2C[C@@H]4CN(CCCOc5ccc6c(c5)C(=O)N(C5CCC(=O)NC5=O)C6=O)C[C@H]24)C3)cc(OC)c1CN(C)C] |
| F247 | 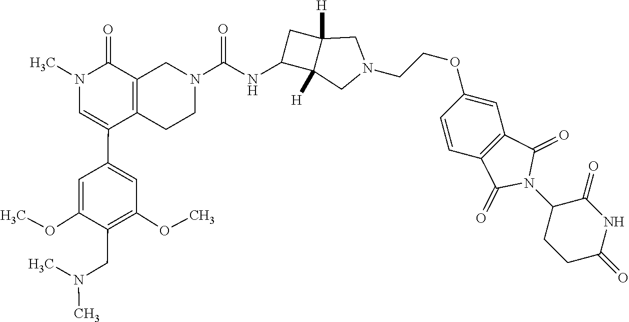 [SMILES: COc1cc(-c2cn(C)c(=O)c3c2CCN(C(=O)NC2C[C@@H]4CN(CCOc5ccc6c(c5)C(=O)N(C5CCC(=O)NC5=O)C6=O)C[C@H]24)C3)cc(OC)c1CN(C)C] |
| F248 | 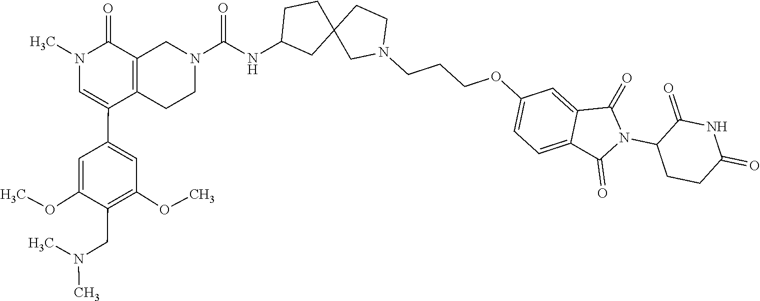 [SMILES: COc1cc(-c2cn(C)c(=O)c3c2CCN(C(=O)NC2CCC4(CCN(CCCOc5ccc6c(c5)C(=O)N(C5CCC(=O)NC5=O)C6=O)C4)C2)C3)cc(OC)c1CN(C)C] |
| F249 | 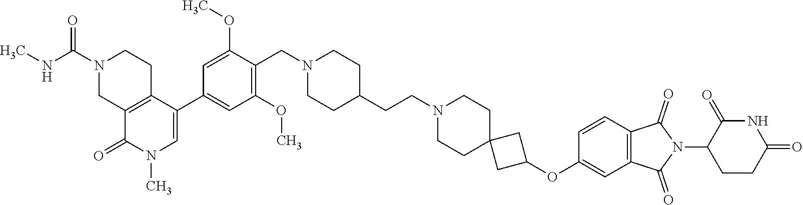 [SMILES: CNC(=O)N1CCc2c(-c3cc(OC)c(CN4CCC(CCN5CCC6(CC5)CC(Oc5ccc7c(c5)C(=O)N(C5CCC(=O)NC5=O)C7=O)C6)CC4)c(OC)c3)cn(C)c(=O)c2C1] |
| F250 | 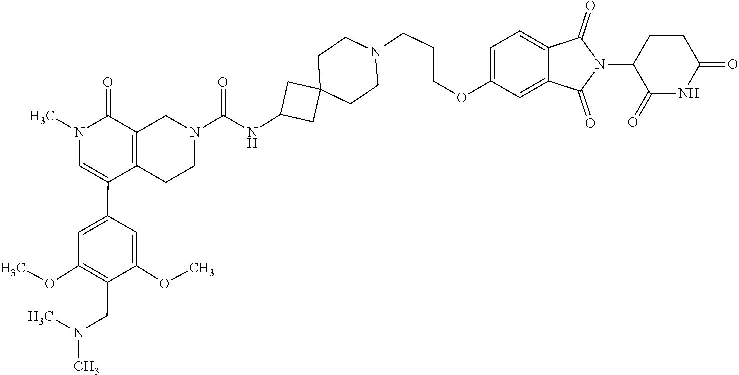 [SMILES: COc1cc(-c2cn(C)c(=O)c3c2CCN(C(=O)NC2CC4(CCN(CCCOc5ccc6c(c5)C(=O)N(C5CCC(=O)NC5=O)C6=O)CC4)C2)C3)cc(OC)c1CN(C)C] |
| F251 | 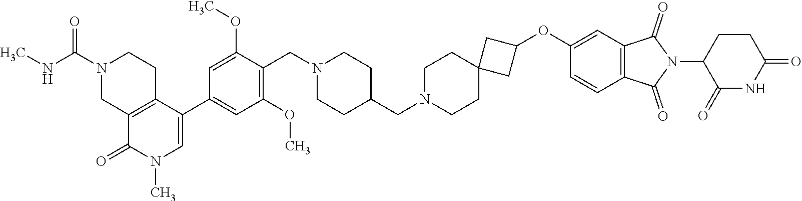 [SMILES: CNC(=O)N1CCc2c(-c3cc(OC)c(CN4CCC(CN5CCC6(CC5)CC(Oc5ccc7c(c5)C(=O)N(C5CCC(=O)NC5=O)C7=O)C6)CC4)c(OC)c3)cn(C)c(=O)c2C1] |
| F252 | 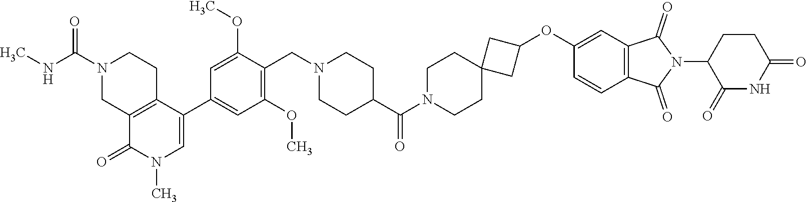 [SMILES: CNC(=O)N1CCc2c(-c3cc(OC)c(CN4CCC(C(=O)N5CCC6(CC5)CC(Oc5ccc7c(c5)C(=O)N(C5CCC(=O)NC5=O)C7=O)C6)CC4)c(OC)c3)cn(C)c(=O)c2C1] |
| F253 | 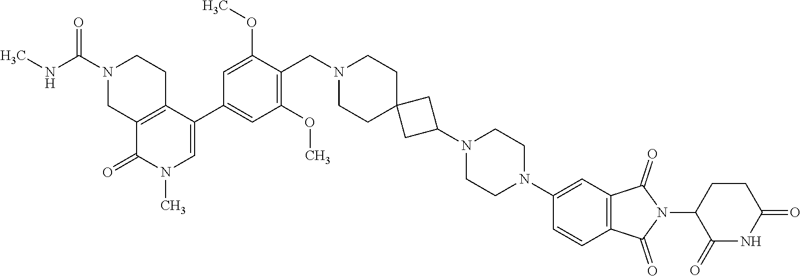 [SMILES: CNC(=O)N1CCc2c(-c3cc(OC)c(CN4CCC5(CC4)CC(N4CCN(c6ccc7c(c6)C(=O)N(C6CCC(=O)NC6=O)C7=O)CC4)C5)c(OC)c3)cn(C)c(=O)c2C1] |
| F254 | 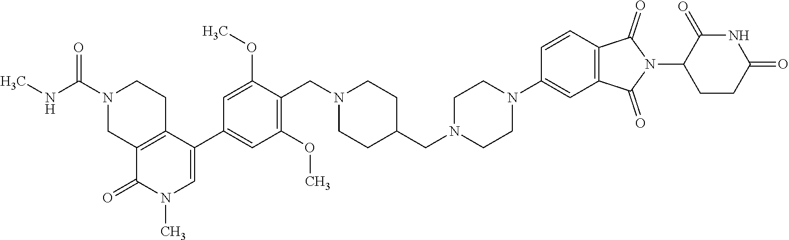 [SMILES: CNC(=O)N1CCc2c(-c3cc(OC)c(CN4CCC(CN5CCN(c6ccc7c(c6)C(=O)N(C6CCC(=O)NC6=O)C7=O)CC5)CC4)c(OC)c3)cn(C)c(=O)c2C1] |
| F255 | 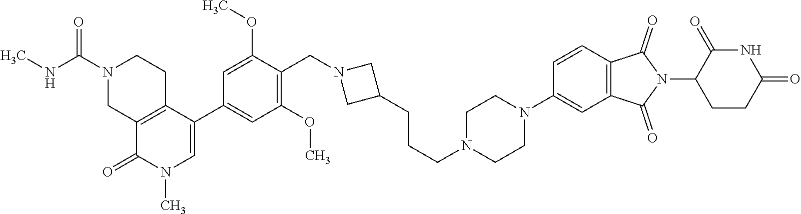 [SMILES: CNC(=O)N1CCc2c(-c3cc(OC)c(CN4CC(CCCN5CCN(c6ccc7c(c6)C(=O)N(C6CCC(=O)NC6=O)C7=O)CC5)C4)c(OC)c3)cn(C)c(=O)c2C1] |
| F256 | 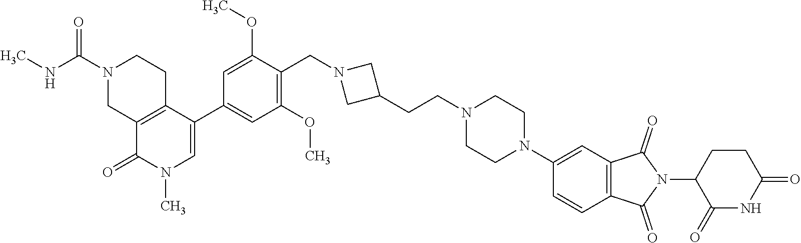 [SMILES: CNC(=O)N1CCc2c(-c3cc(OC)c(CN4CC(CCN5CCN(c6ccc7c(c6)C(=O)N(C6CCC(=O)NC6=O)C7=O)CC5)C4)c(OC)c3)cn(C)c(=O)c2C1] |
| F257 | 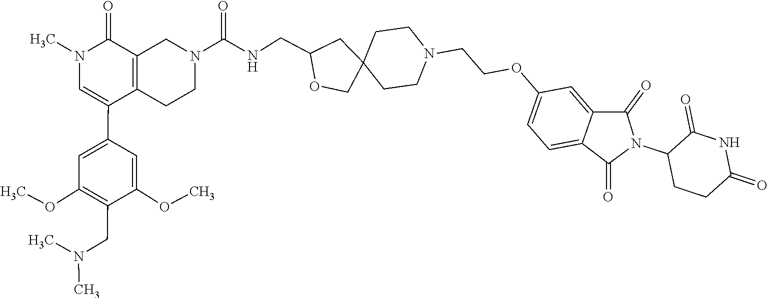 [SMILES: COc1cc(-c2cn(C)c(=O)c3c2CCN(C(=O)NCC2CC4(CCN(CCOc5ccc6c(c5)C(=O)N(C5CCC(=O)NC5=O)C6=O)CC4)CO2)C3)cc(OC)c1CN(C)C] |
| F258 | 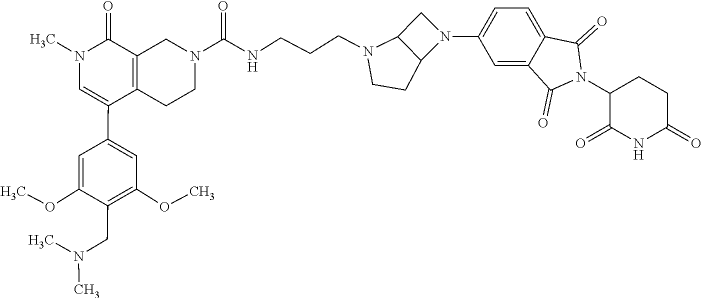 [SMILES: COc1cc(-c2cn(C)c(=O)c3c2CCN(C(=O)NCCCN2CCC4C2CN4c2ccc4c(c2)C(=O)N(C2CCC(=O)NC2=O)C4=O)C3)cc(OC)c1CN(C)C] |
| F259 | 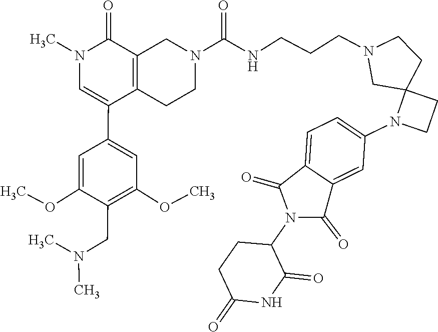 [SMILES: COc1cc(-c2cn(C)c(=O)c3c2CCN(C(=O)NCCCN2CCC4(CCN4c4ccc5c(c4)C(=O)N(C4CCC(=O)NC4=O)C5=O)C2)C3)cc(OC)c1CN(C)C] |
| F260 | 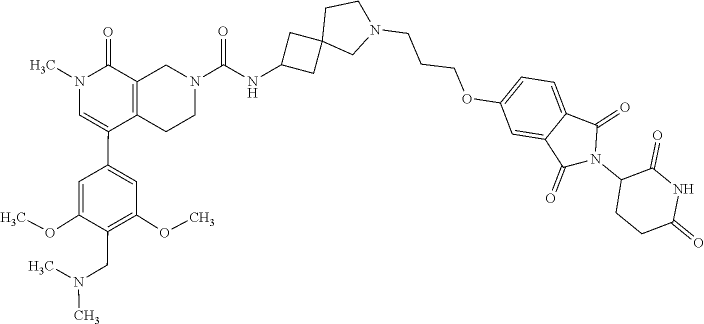 [SMILES: COc1cc(-c2cn(C)c(=O)c3c2CCN(C(=O)NC2CC4(CCN(CCCOc5ccc6c(c5)C(=O)N(C5CCC(=O)NC5=O)C6=O)C4)C2)C3)cc(OC)c1CN(C)C] |
| F261 | 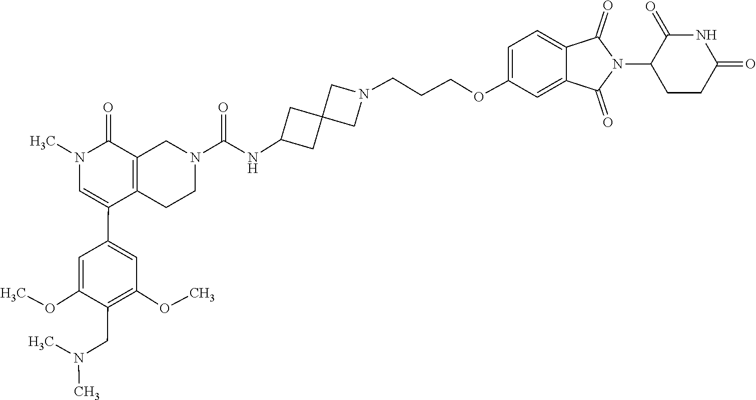 [SMILES: COc1cc(-c2cn(C)c(=O)c3c2CCN(C(=O)NC2CC4(C2)CN(CCCOc2ccc5c(c2)C(=O)N(C2CCC(=O)NC2=O)C5=O)C4)C3)cc(OC)c1CN(C)C] |
| F262 | 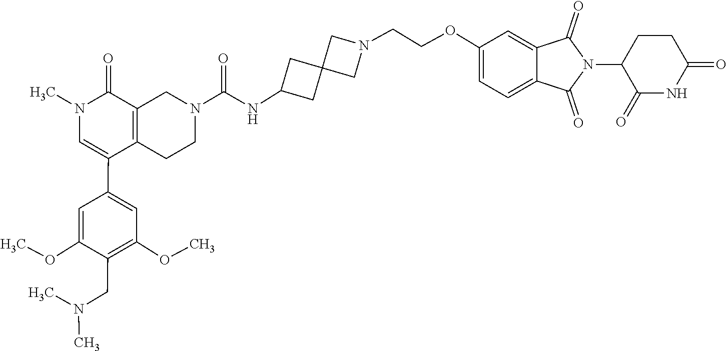 [SMILES: COc1cc(-c2cn(C)c(=O)c3c2CCN(C(=O)NC2CC4(C2)CN(CCOc2ccc5c(c2)C(=O)N(C2CCC(=O)NC2=O)C5=O)C4)C3)cc(OC)c1CN(C)C] |
| F263 | 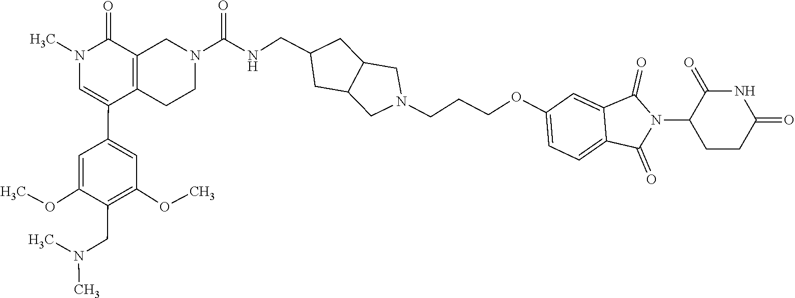 [SMILES: COc1cc(-c2cn(C)c(=O)c3c2CCN(C(=O)NCC2CC4CN(CCCOc5ccc6c(c5)C(=O)N(C5CCC(=O)NC5=O)C6=O)CC4C2)C3)cc(OC)c1CN(C)C] |
| F264 | 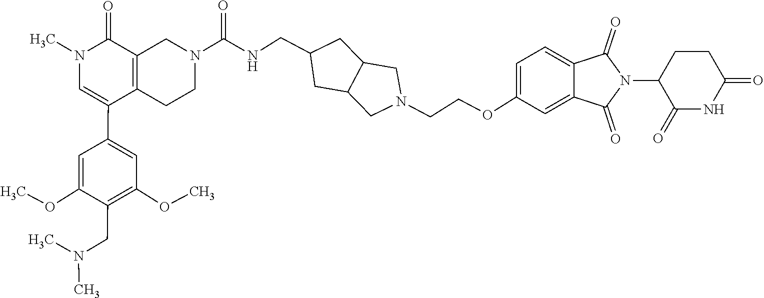 [SMILES: COc1cc(-c2cn(C)c(=O)c3c2CCN(C(=O)NCC2CC4CN(CCOc5ccc6c(c5)C(=O)N(C5CCC(=O)NC5=O)C6=O)CC4C2)C3)cc(OC)c1CN(C)C] |
| F265 | 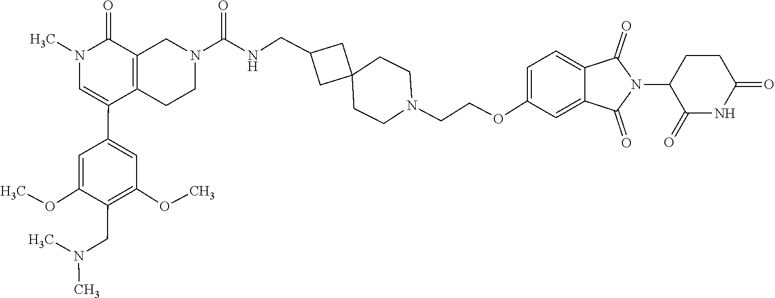 [SMILES: COc1cc(-c2cn(C)c(=O)c3c2CCN(C(=O)NCC2CC4(CCN(CCOc5ccc6c(c5)C(=O)N(C5CCC(=O)NC5=O)C6=O)CC4)C2)C3)cc(OC)c1CN(C)C] |
| F266 | 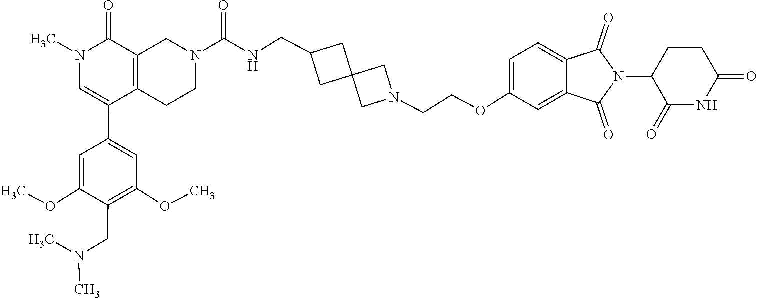 [SMILES: COc1cc(-c2cn(C)c(=O)c3c2CCN(C(=O)NCC2CC4(C2)CN(CCOc2ccc5c(c2)C(=O)N(C2CCC(=O)NC2=O)C5=O)C4)C3)cc(OC)c1CN(C)C] |
| F267 | 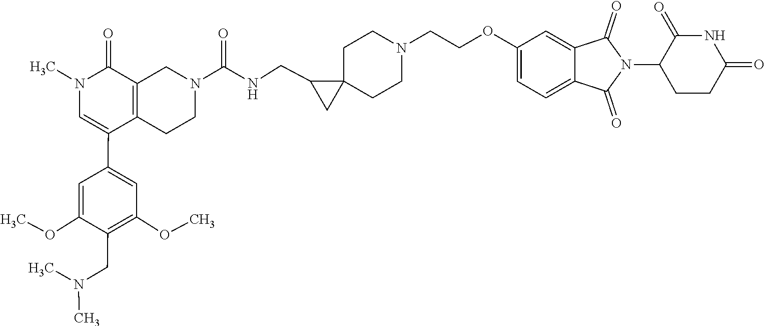 [SMILES: COc1cc(-c2cn(C)c(=O)c3c2CCN(C(=O)NCC2CC24CCN(CCOc2ccc5c(c2)C(=O)N(C2CCC(=O)NC2=O)C5=O)CC4)C3)cc(OC)c1CN(C)C] |
| F268 | 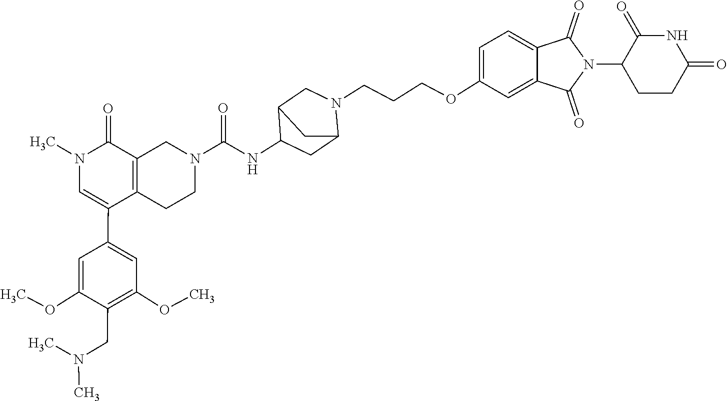 [SMILES: COc1cc(-c2cn(C)c(=O)c3c2CCN(C(=O)NC2CC4CC2CN4CCCOc2ccc4c(c2)C(=O)N(C2CCC(=O)NC2=O)C4=O)C3)cc(OC)c1CN(C)C] |
| F269 | 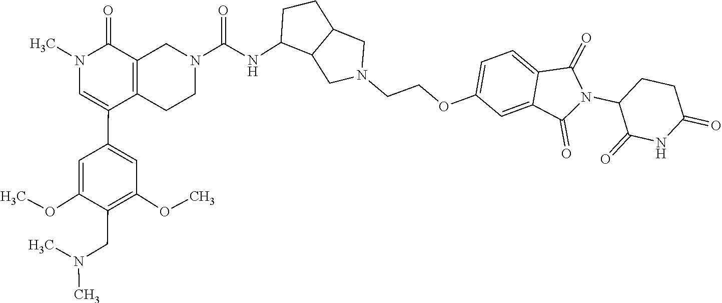 [SMILES: COc1cc(-c2cn(C)c(=O)c3c2CCN(C(=O)NC2CCC4CN(CCOc5ccc6c(c5)C(=O)N(C5CCC(=O)NC5=O)C6=O)CC42)C3)cc(OC)c1CN(C)C] |
| F270 | 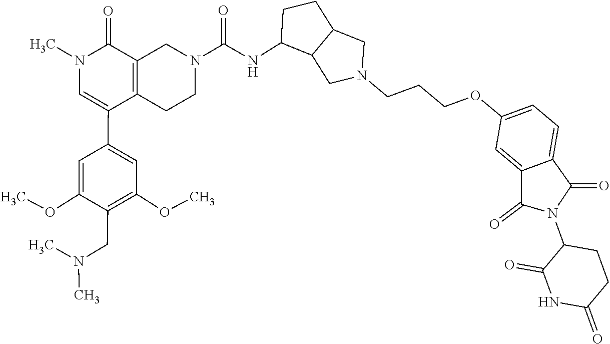 [SMILES: COc1cc(-c2cn(C)c(=O)c3c2CCN(C(=O)NC2CCC4CN(CCCOc5ccc6c(c5)C(=O)N(C5CCC(=O)NC5=O)C6=O)CC42)C3)cc(OC)c1CN(C)C] |
| F271 | 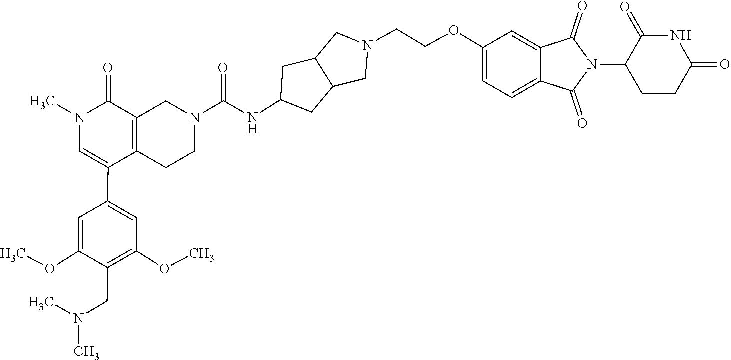 [SMILES: COc1cc(-c2cn(C)c(=O)c3c2CCN(C(=O)NC2CC4CN(CCOc5ccc6c(c5)C(=O)N(C5CCC(=O)NC5=O)C6=O)CC4C2)C3)cc(OC)c1CN(C)C] |
| F272 | 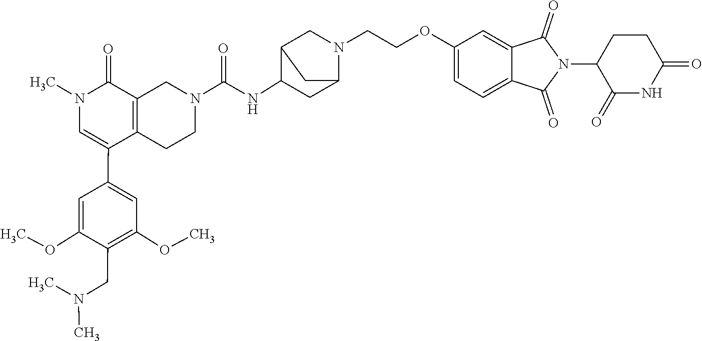 [SMILES: COc1cc(-c2cn(C)c(=O)c3c2CCN(C(=O)NC2CC4CC2CN4CCOc2ccc4c(c2)C(=O)N(C2CCC(=O)NC2=O)C4=O)C3)cc(OC)c1CN(C)C] |
| F273 | 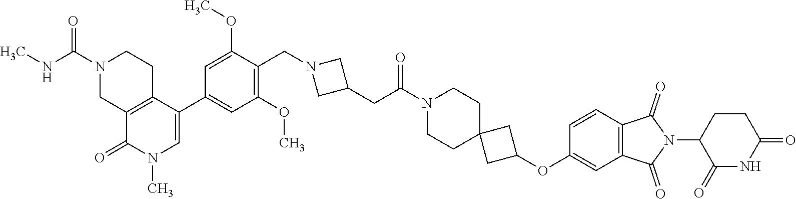 [SMILES: CNC(=O)N1CCc2c(-c3cc(OC)c(CN4CC(CC(=O)N5CCC6(CC5)CC(Oc5ccc7c(c5)C(=O)N(C5CCC(=O)NC5=O)C7=O)C6)C4)c(OC)c3)cn(C)c(=O)c2C1] |
| F274 | 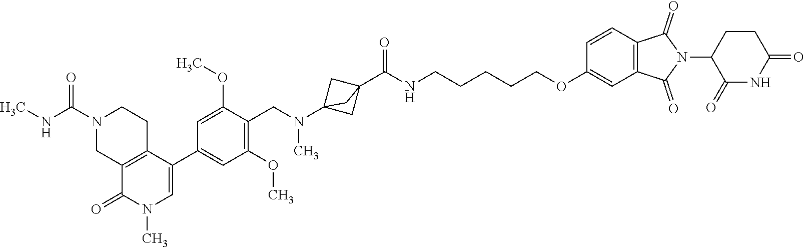 [SMILES: CNC(=O)N1CCc2c(-c3cc(OC)c(CN(C)C45CC(C(=O)NCCCCCOc6ccc7c(c6)C(=O)N(C6CCC(=O)NC6=O)C7=O)(C4)C5)c(OC)c3)cn(C)c(=O)c2C1] |
| F275 | 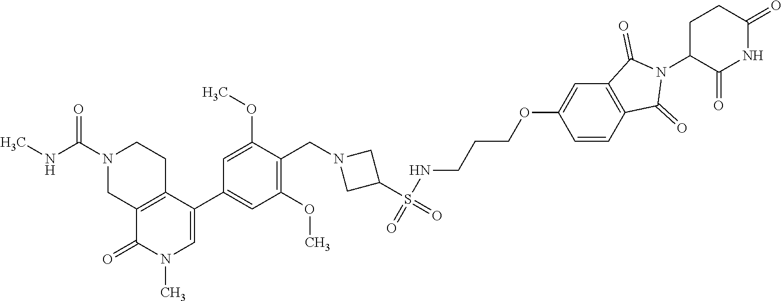 [SMILES: CNC(=O)N1CCc2c(-c3cc(OC)c(CN4CC(S(=O)(=O)NCCCOc5ccc6c(c5)C(=O)N(C5CCC(=O)NC5=O)C6=O)C4)c(OC)c3)cn(C)c(=O)c2C1] |
| F276 | 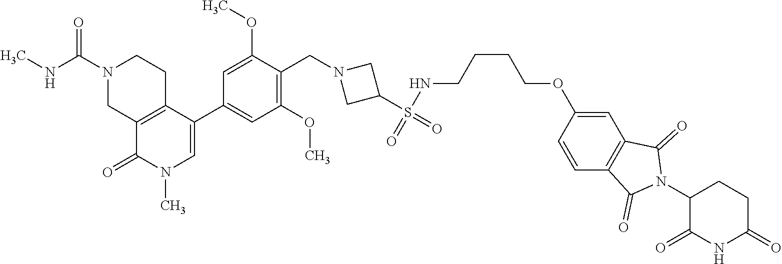 [SMILES: CNC(=O)N1CCc2c(-c3cc(OC)c(CN4CC(S(=O)(=O)NCCCCOc5ccc6c(c5)C(=O)N(C5CCC(=O)NC5=O)C6=O)C4)c(OC)c3)cn(C)c(=O)c2C1] |
| F277 | 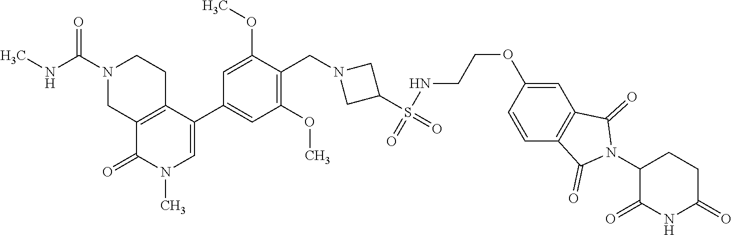 [SMILES: CNC(=O)N1CCc2c(-c3cc(OC)c(CN4CC(S(=O)(=O)NCCOc5ccc6c(c5)C(=O)N(C5CCC(=O)NC5=O)C6=O)C4)c(OC)c3)cn(C)c(=O)c2C1] |
| F278 | 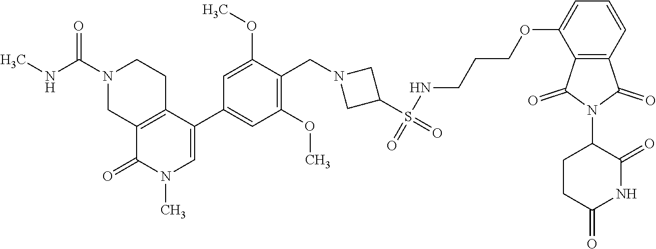 [SMILES: CNC(=O)N1CCc2c(-c3cc(OC)c(CN4CC(S(=O)(=O)NCCCOc5cccc6c5C(=O)N(C5CCC(=O)NC5=O)C6=O)C4)c(OC)c3)cn(C)c(=O)c2C1] |
| F279 | 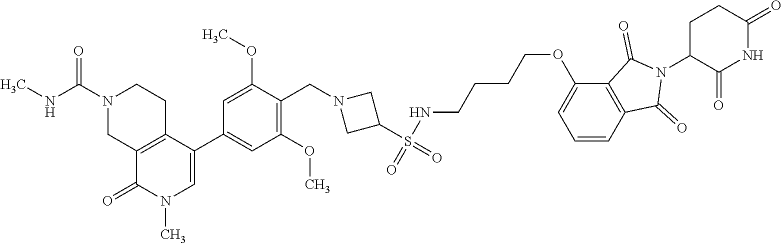 [SMILES: CNC(=O)N1CCc2c(-c3cc(OC)c(CN4CC(S(=O)(=O)NCCCCOc5cccc6c5C(=O)N(C5CCC(=O)NC5=O)C6=O)C4)c(OC)c3)cn(C)c(=O)c2C1] |
| F280 | 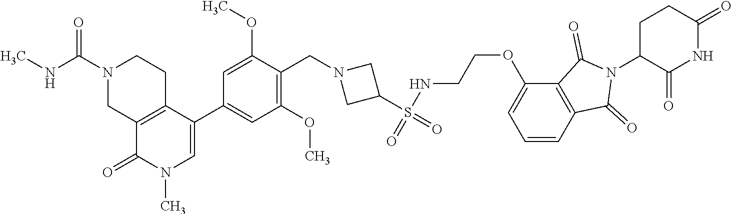 [SMILES: CNC(=O)N1CCc2c(-c3cc(OC)c(CN4CC(S(=O)(=O)NCCOc5cccc6c5C(=O)N(C5CCC(=O)NC5=O)C6=O)C4)c(OC)c3)cn(C)c(=O)c2C1] |
| F281 | 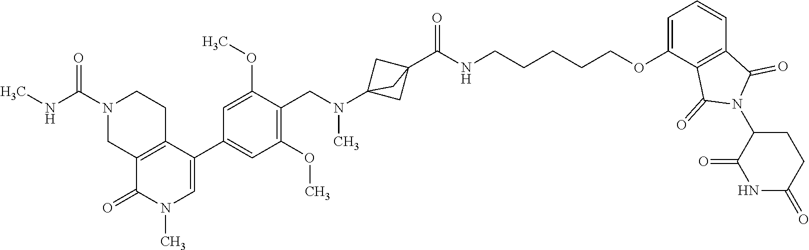 [SMILES: CNC(=O)N1CCc2c(-c3cc(OC)c(CN(C)C45CC(C(=O)NCCCCCOc6cccc7c6C(=O)N(C6CCC(=O)NC6=O)C7=O)(C4)C5)c(OC)c3)cn(C)c(=O)c2C1] |
| F282 | 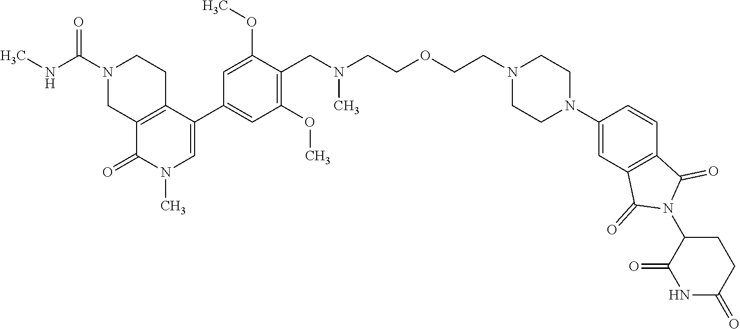 [SMILES: CNC(=O)N1CCc2c(-c3cc(OC)c(CN(C)CCOCCN4CCN(c5ccc6c(c5)C(=O)N(C5CCC(=O)NC5=O)C6=O)CC4)c(OC)c3)cn(C)c(=O)c2C1] |
| F283 | 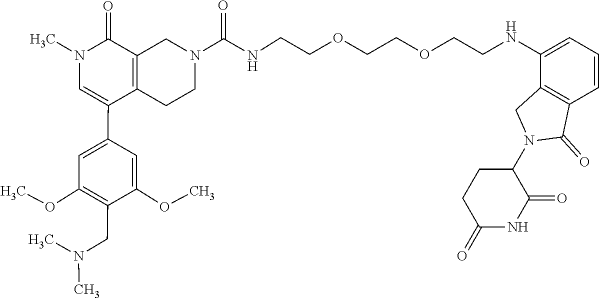 [SMILES: COc1cc(-c2cn(C)c(=O)c3c2CCN(C(=O)NCCOCCOCCNc2cccc4c2CN(C2CCC(=O)NC2=O)C4=O)C3)cc(OC)c1CN(C)C] |
| F284 | 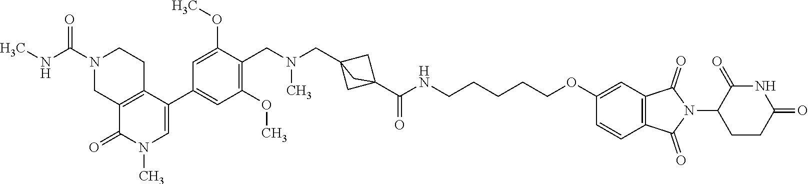 [SMILES: CNC(=O)N1CCc2c(-c3cc(OC)c(CN(C)CC45CC(C(=O)NCCCCCOc6ccc7c(c6)C(=O)N(C6CCC(=O)NC6=O)C7=O)(C4)C5)c(OC)c3)cn(C)c(=O)c2C1] |
| F285 | 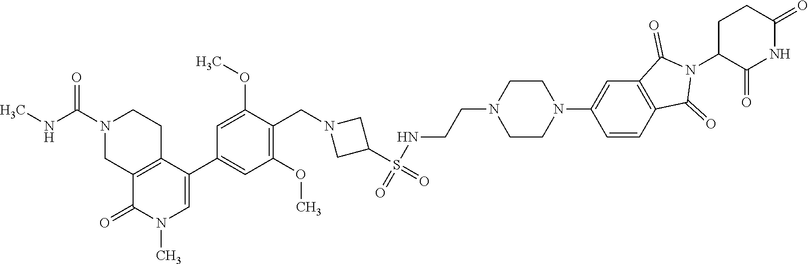 [SMILES: CNC(=O)N1CCc2c(-c3cc(OC)c(CN4CC(S(=O)(=O)NCCN5CCN(c6ccc7c(c6)C(=O)N(C6CCC(=O)NC6=O)C7=O)CC5)C4)c(OC)c3)cn(C)c(=O)c2C1] |
| F286 | 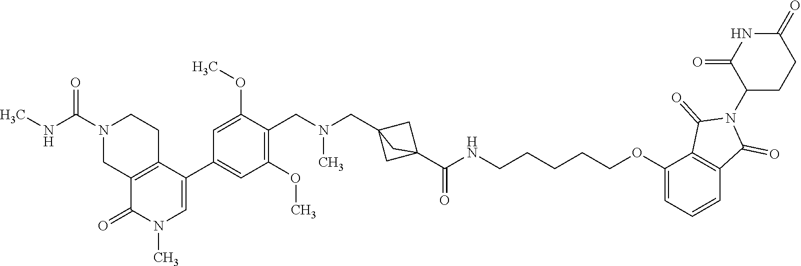 [SMILES: CNC(=O)N1CCc2c(-c3cc(OC)c(CN(C)CC45CC(C(=O)NCCCCCOc6cccc7c6C(=O)N(C6CCC(=O)NC6=O)C7=O)(C4)C5)c(OC)c3)cn(C)c(=O)c2C1] |
| F287 | 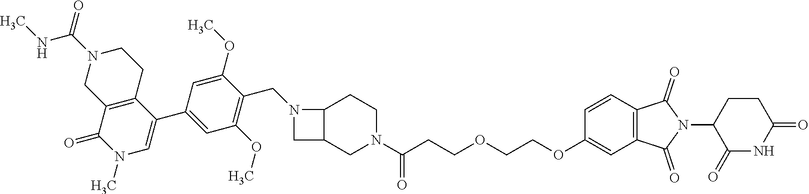 [SMILES: CNC(=O)N1CCc2c(-c3cc(OC)c(CN4CC5CN(C(=O)CCOCCOc6ccc7c(c6)C(=O)N(C6CCC(=O)NC6=O)C7=O)CCC54)c(OC)c3)cn(C)c(=O)c2C1] |
| F288 | 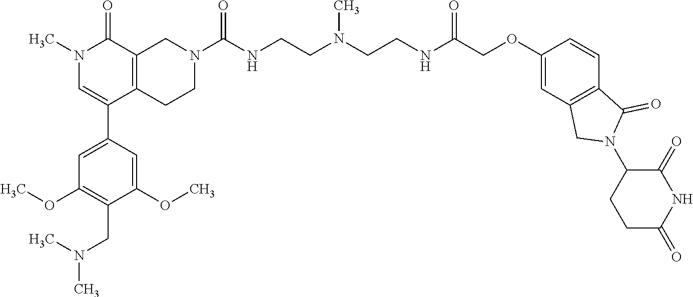 [SMILES: COc1cc(-c2cn(C)c(=O)c3c2CCN(C(=O)NCCN(C)CCNC(=O)COc2ccc4c(c2)CN(C2CCC(=O)NC2=O)C4=O)C3)cc(OC)c1CN(C)C] |
| F289 | 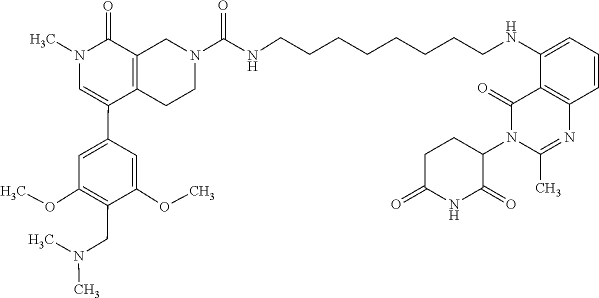 [SMILES: COc1cc(-c2cn(C)c(=O)c3c2CCN(C(=O)NCCCCCCCCNc2cccc4nc(C)n(C5CCC(=O)NC5=O)c(=O)c24)C3)cc(OC)c1CN(C)C] |
| F290 | 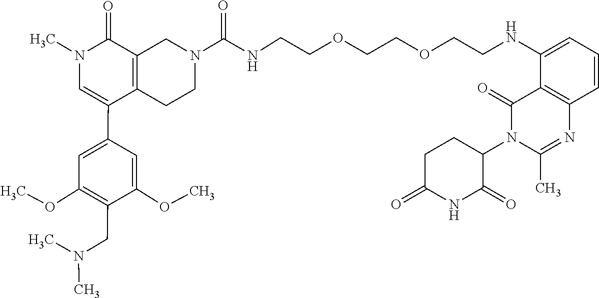 [SMILES: COc1cc(-c2cn(C)c(=O)c3c2CCN(C(=O)NCCOCCOCCNc2cccc4nc(C)n(C5CCC(=O)NC5=O)c(=O)c24)C3)cc(OC)c1CN(C)C] |
| F291 | 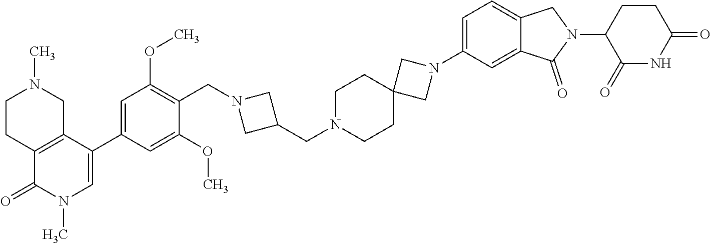 [SMILES: COc1cc(-c2cn(C)c(=O)c3c2CN(C)CC3)cc(OC)c1CN1CC(CN2CCC3(CC2)CN(c2ccc4c(c2)C(=O)N(C2CCC(=O)NC2=O)C4)C3)C1] |
| F292 | 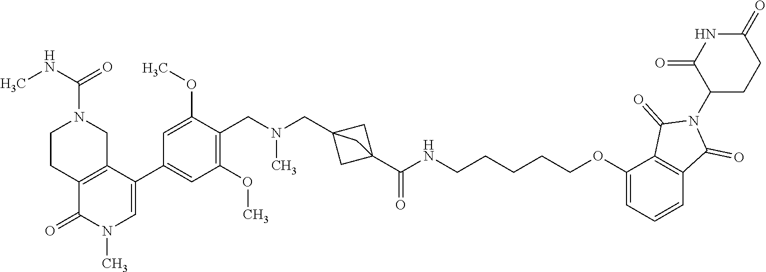 [SMILES: CNC(=O)N1CCc2c(c(-c3cc(OC)c(CN(C)CC45CC(C(=O)NCCCCCOc6cccc7c6C(=O)N(C6CCC(=O)NC6=O)C7=O)(C4)C5)c(OC)c3)cn(C)c2=O)C1] |
| F293 | 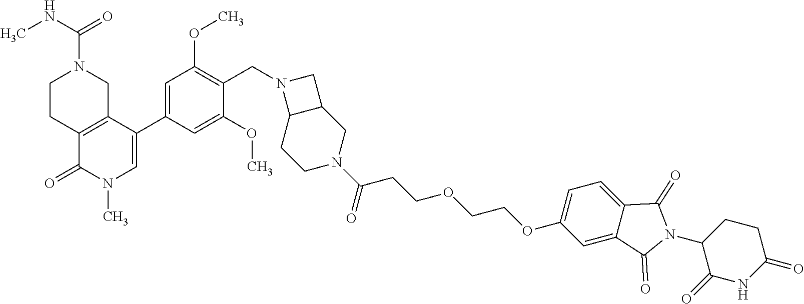 [SMILES: CNC(=O)N1CCc2c(c(-c3cc(OC)c(CN4CC5CN(C(=O)CCOCCOc6ccc7c(c6)C(=O)N(C6CCC(=O)NC6=O)C7=O)CCC54)c(OC)c3)cn(C)c2=O)C1] |
| F294 | 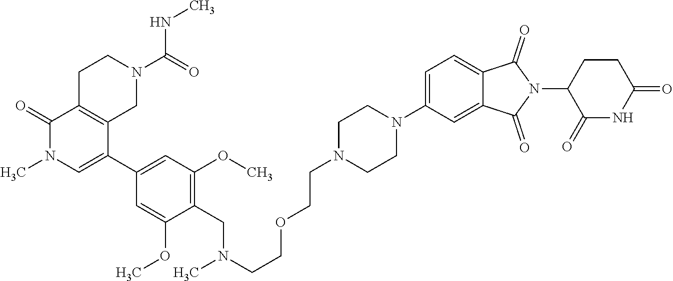 [SMILES: CNC(=O)N1CCc2c(c(-c3cc(OC)c(CN(C)CCOCCN4CCN(c5ccc6c(c5)C(=O)N(C5CCC(=O)NC5=O)C6=O)CC4)c(OC)c3)cn(C)c2=O)C1] |
| F295 | 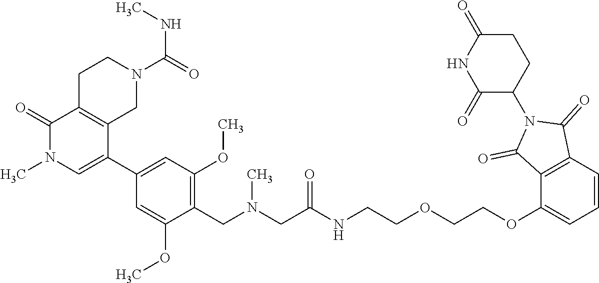 [SMILES: CNC(=O)N1CCc2c(c(-c3cc(OC)c(CN(C)CC(=O)NCCOCCOc4cccc5c4C(=O)N(C4CCC(=O)NC4=O)C5=O)c(OC)c3)cn(C)c2=O)C1] |
| F296 | 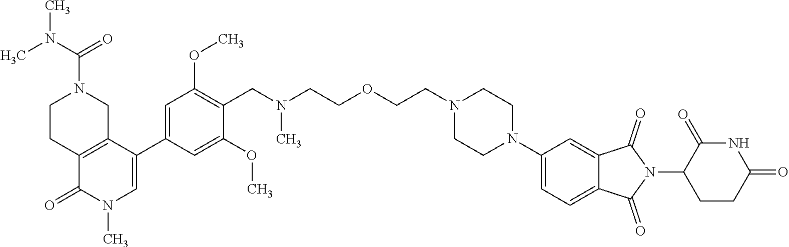 [SMILES: COc1cc(-c2cn(C)c(=O)c3c2CN(C(=O)N(C)C)CC3)cc(OC)c1CN(C)CCOCCN1CCN(c2ccc3c(c2)C(=O)N(C2CCC(=O)NC2=O)C3=O)CC1] |
| F297 | 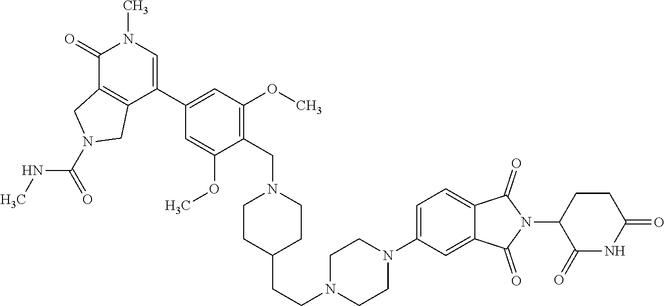 [SMILES: CNC(=O)N1Cc2c(-c3cc(OC)c(CN4CCC(CCN5CCN(c6ccc7c(c6)C(=O)N(C6CCC(=O)NC6=O)C7=O)CC5)CC4)c(OC)c3)cn(C)c(=O)c2C1] |
| F298 | 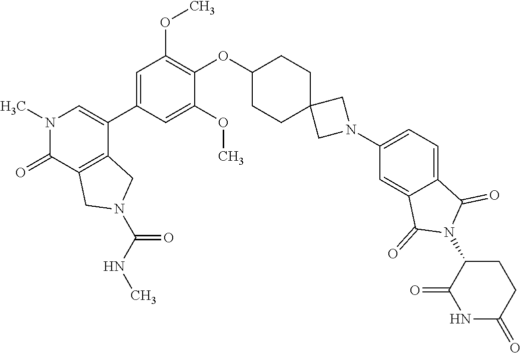 [SMILES: CNC(=O)N1Cc2c(-c3cc(OC)c(OC4CCC5(CC4)CN(c4ccc6c(c4)C(=O)N([C@@H]4CCC(=O)NC4=O)C6=O)C5)c(OC)c3)cn(C)c(=O)c2C1] |
| F299 | 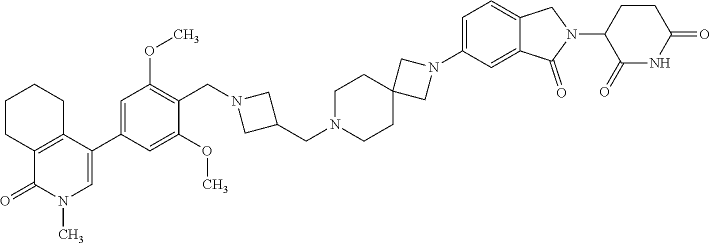 [SMILES: COc1cc(-c2cn(C)c(=O)c3c2CCCC3)cc(OC)c1CN1CC(CN2CCC3(CC2)CN(c2ccc4c(c2)C(=O)N(C2CCC(=O)NC2=O)C4)C3)C1] |
| F300 | 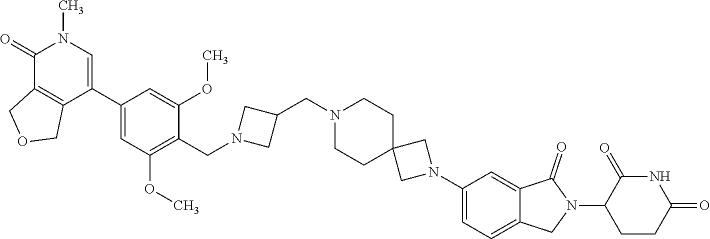 [SMILES: COc1cc(-c2cn(C)c(=O)c3c2COC3)cc(OC)c1CN1CC(CN2CCC3(CC2)CN(c2ccc4c(c2)C(=O)N(C2CCC(=O)NC2=O)C4)C3)C1] |
| F301 | 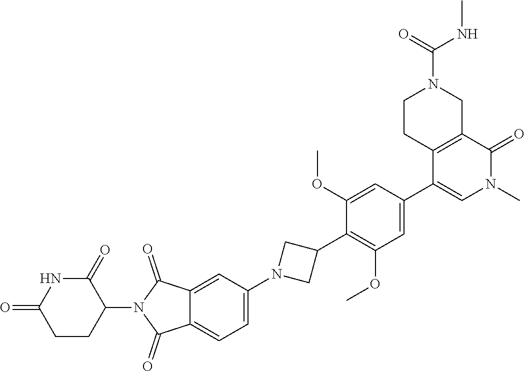 [SMILES: CNC(=O)N1CCc2c(-c3cc(OC)c(C4CN(c5ccc6c(c5)C(=O)N(C5CCC(=O)NC5=O)C6=O)C4)c(OC)c3)cn(C)c(=O)c2C1] |
| F302 | 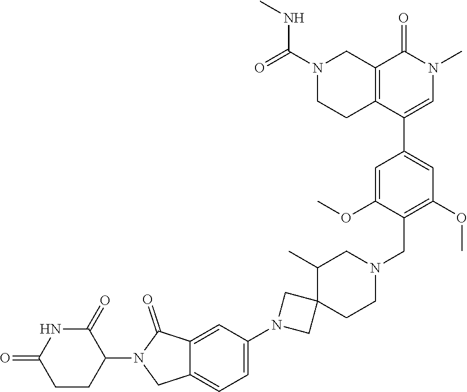 [SMILES: CNC(=O)N1CCc2c(-c3cc(OC)c(CN4CCC5(CN(c6ccc7c(c6)C(=O)N(C6CCC(=O)NC6=O)C7)C5)C(C)C4)c(OC)c3)cn(C)c(=O)c2C1] |
| F303 | 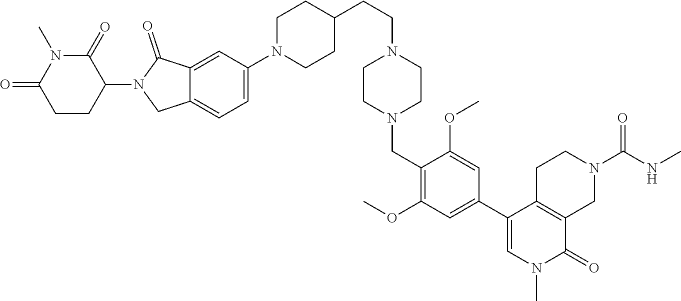 [SMILES: CNC(=O)N1CCc2c(-c3cc(OC)c(CN4CCN(CCC5CCN(c6ccc7c(c6)C(=O)N(C6CCC(=O)N(C)C6=O)C7)CC5)CC4)c(OC)c3)cn(C)c(=O)c2C1] |
| F304 | 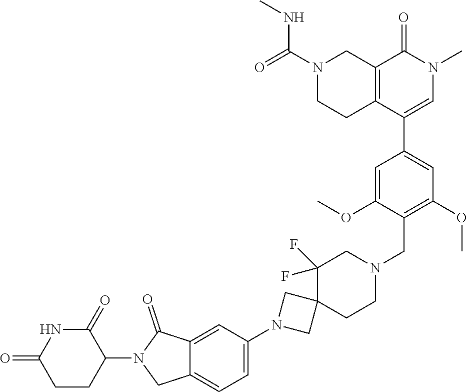 [SMILES: CNC(=O)N1CCc2c(-c3cc(OC)c(CN4CCC5(CN(c6ccc7c(c6)C(=O)N(C6CCC(=O)NC6=O)C7)C5)C(F)(F)C4)c(OC)c3)cn(C)c(=O)c2C1] |
| F305 | 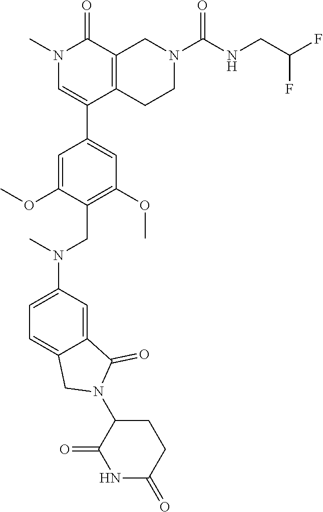 [SMILES: COc1cc(-c2cn(C)c(=O)c3c2CCN(C(=O)NCC(F)F)C3)cc(OC)c1CN(C)c1ccc2c(c1)C(=O)N(C1CCC(=O)NC1=O)C2] |
| F306 | 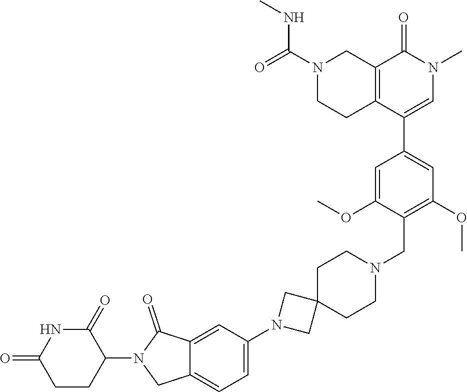 [SMILES: CNC(=O)N1CCc2c(-c3cc(OC)c(CN4CCC5(CC4)CN(c4ccc6c(c4)C(=O)N(C4CCC(=O)NC4=O)C6)C5)c(OC)c3)cn(C)c(=O)c2C1] |
| F307 | 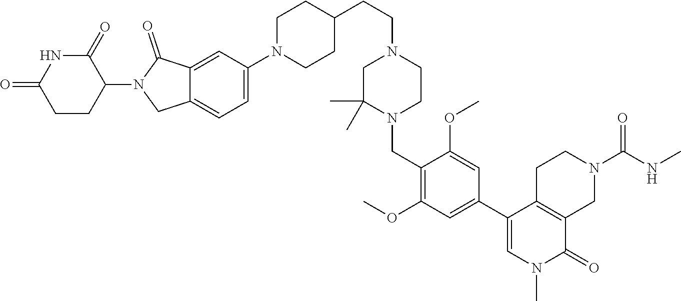 [SMILES: CNC(=O)N1CCc2c(-c3cc(OC)c(CN4CCN(CCC5CCN(c6ccc7c(c6)C(=O)N(C6CCC(=O)NC6=O)C7)CC5)CC4(C)C)c(OC)c3)cn(C)c(=O)c2C1] |
| F308 | 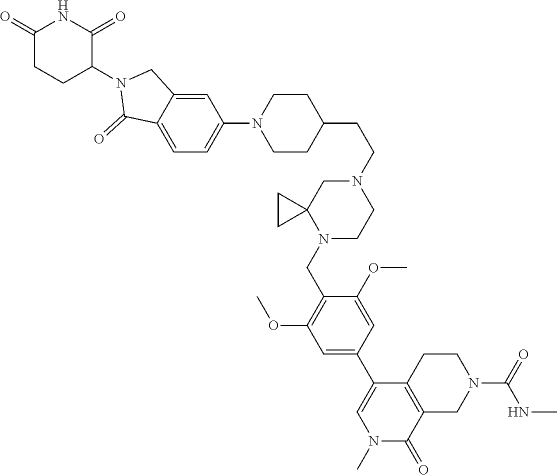 [SMILES: CNC(=O)N1CCc2c(-c3cc(OC)c(CN4CCN(CCC5CCN(c6ccc7c(c6)CN(C6CCC(=O)NC6=O)C7=O)CC5)CC45CC5)c(OC)c3)cn(C)c(=O)c2C1] |
| F309 | 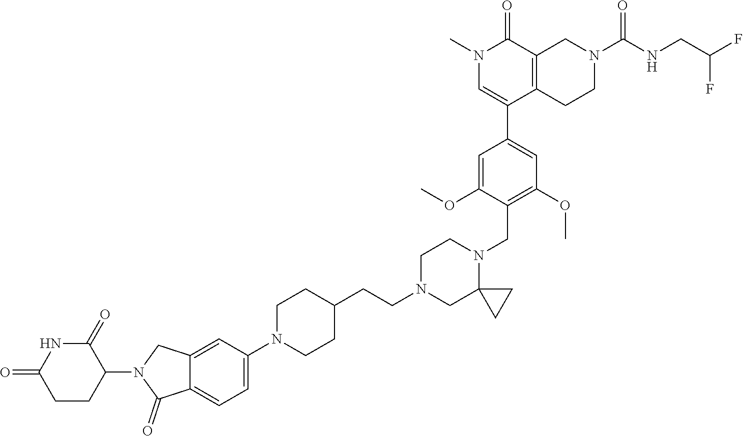 [SMILES: COc1cc(-c2cn(C)c(=O)c3c2CCN(C(=O)NCC(F)F)C3)cc(OC)c1CN1CCN(CCC2CCN(c3ccc4c(c3)CN(C3CCC(=O)NC3=O)C4=O)CC2)CC12CC2] |
| F310 | 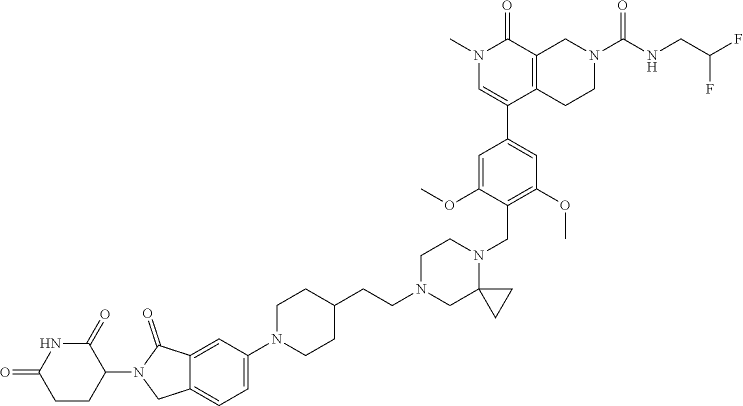 [SMILES: COc1cc(-c2cn(C)c(=O)c3c2CCN(C(=O)NCC(F)F)C3)cc(OC)c1CN1CCN(CCC2CCN(c3ccc4c(c3)C(=O)N(C3CCC(=O)NC3=O)C4)CC2)CC12CC2] |
| F311 | 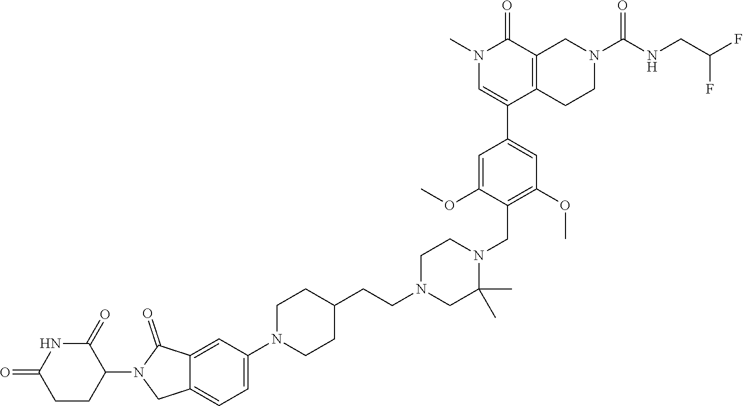 [SMILES: COc1cc(-c2cn(C)c(=O)c3c2CCN(C(=O)NCC(F)F)C3)cc(OC)c1CN1CCN(CCC2CCN(c3ccc4c(c3)C(=O)N(C3CCC(=O)NC3=O)C4)CC2)CC1(C)C] |
| F312 | 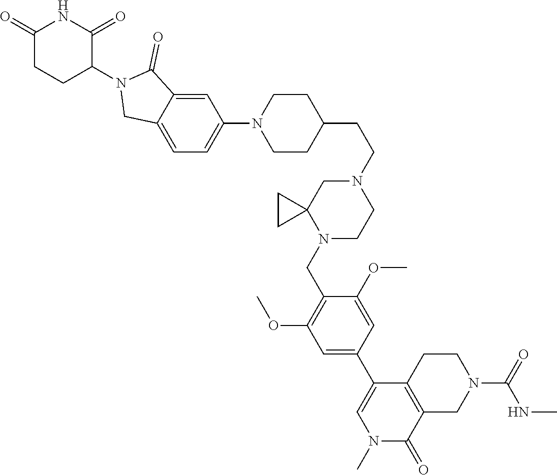 [SMILES: CNC(=O)N1CCc2c(-c3cc(OC)c(CN4CCN(CCC5CCN(c6ccc7c(c6)C(=O)N(C6CCC(=O)NC6=O)C7)CC5)CC45CC5)c(OC)c3)cn(C)c(=O)c2C1] |
| F313 | 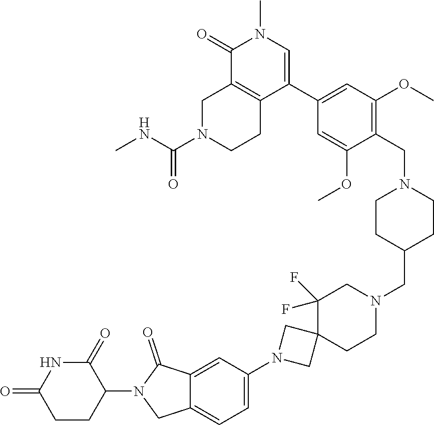 [SMILES: CNC(=O)N1CCc2c(-c3cc(OC)c(CN4CCC(CN5CCC6(CN(c7ccc8c(c7)C(=O)N(C7CCC(=O)NC7=O)C8)C6)C(F)(F)C5)CC4)c(OC)c3)cn(C)c(=O)c2C1] |
| F314 | 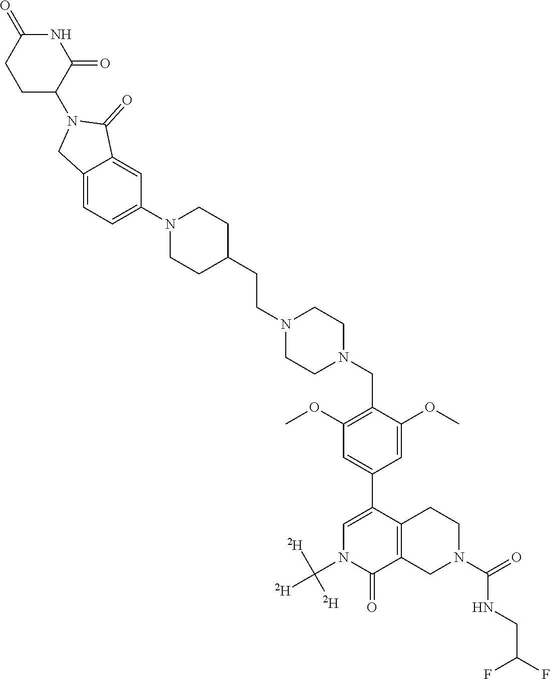 [SMILES: [2H]C([2H])([2H])n1cc(-c2cc(OC)c(CN3CCN(CCC4CCN(c5ccc6c(c5)C(=O)N(C5CCC(=O)NC5=O)C6)CC4)CC3)c(OC)c2)c2c(c1=O)CN(C(=O)NCC(F)F)CC2] |
| F315 | 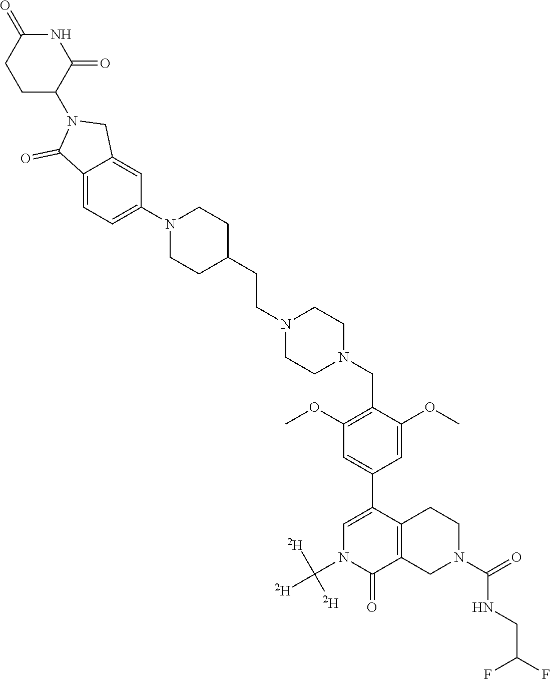 [SMILES: [2H]C([2H])([2H])n1cc(-c2cc(OC)c(CN3CCN(CCC4CCN(c5ccc6c(c5)CN(C5CCC(=O)NC5=O)C6=O)CC4)CC3)c(OC)c2)c2c(c1=O)CN(C(=O)NCC(F)F)CC2] |
| F316 | 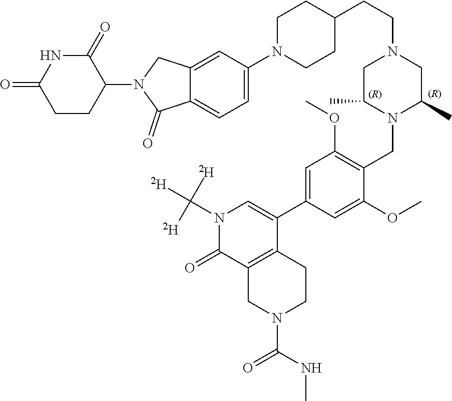 |
| F317 | 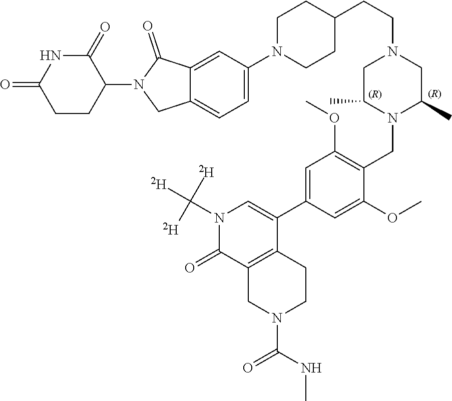 |
| F318 | 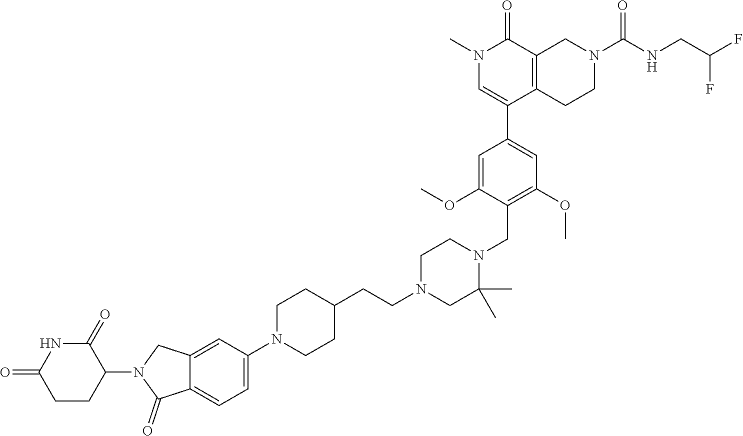 [SMILES: COc1cc(-c2cn(C)c(=O)c3c2CCN(C(=O)NCC(F)F)C3)cc(OC)c1CN1CCN(CCC2CCN(c3ccc4c(c3)CN(C3CCC(=O)NC3=O)C4=O)CC2)CC1(C)C] |
| F319 | 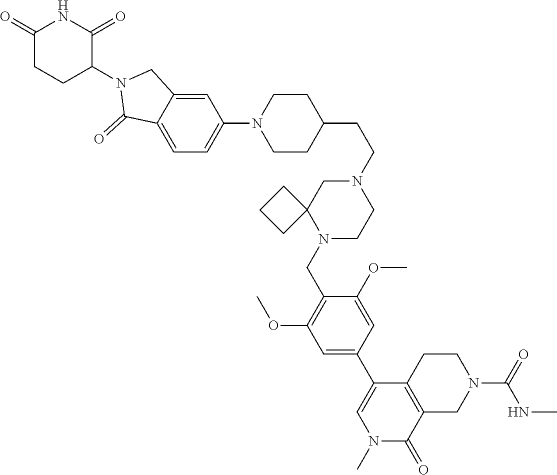 [SMILES: CNC(=O)N1CCc2c(-c3cc(OC)c(CN4CCN(CCC5CCN(c6ccc7c(c6)CN(C6CCC(=O)NC6=O)C7=O)CC5)CC45CCC5)c(OC)c3)cn(C)c(=O)c2C1] |
| F320 | 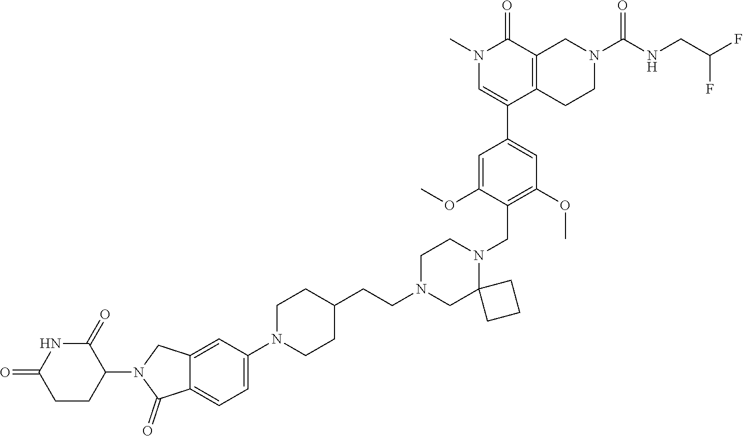 [SMILES: COc1cc(-c2cn(C)c(=O)c3c2CCN(C(=O)NCC(F)F)C3)cc(OC)c1CN1CCN(CCC2CCN(c3ccc4c(c3)CN(C3CCC(=O)NC3=O)C4=O)CC2)CC12CCC2] |
| F321 | 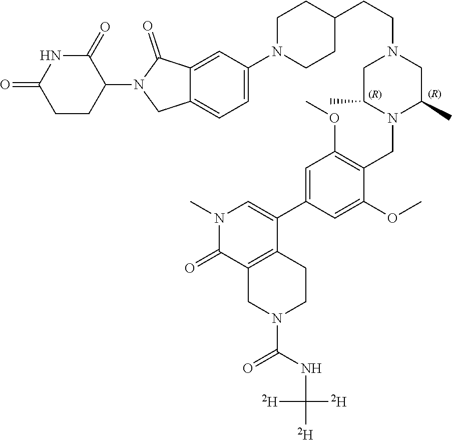 |
| F322 | 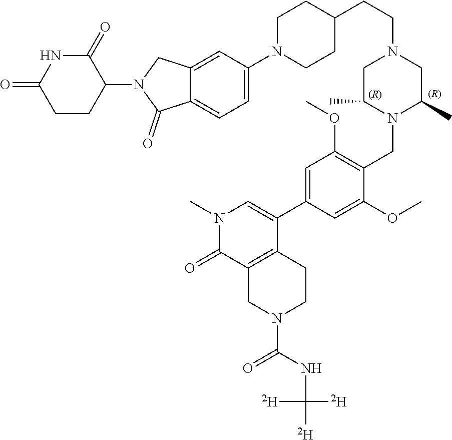 |
| F323 | 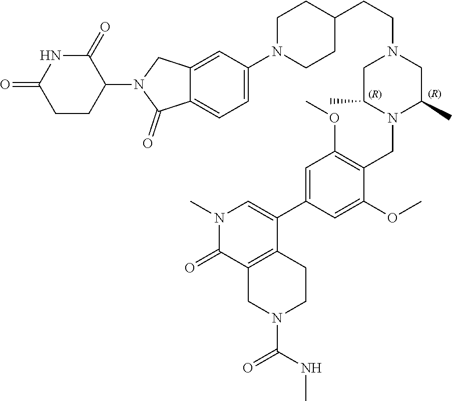 |
| F324 | 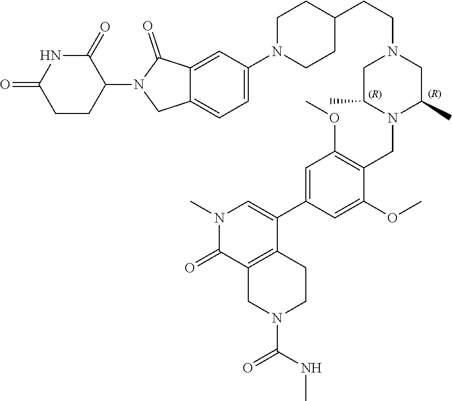 |
| F325 | 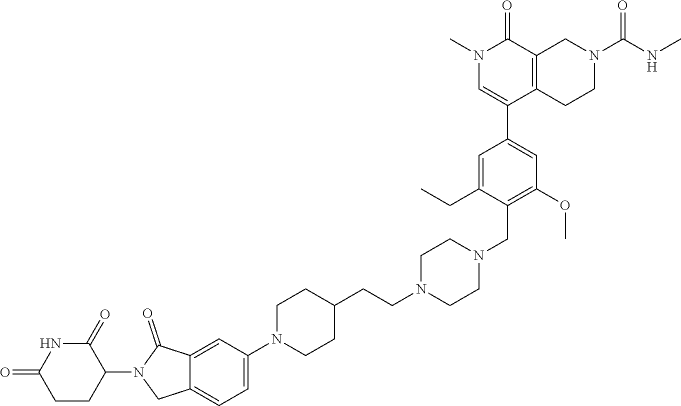 [SMILES: CCc1cc(-c2cn(C)c(=O)c3c2CCN(C(=O)NC)C3)cc(OC)c1CN1CCN(CCC2CCN(c3ccc4c(c3)C(=O)N(C3CCC(=O)NC3=O)C4)CC2)CC1] |
| F326 | 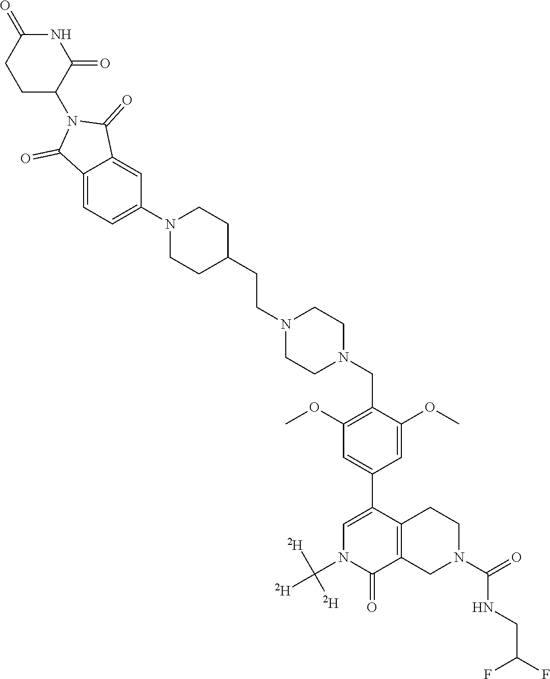 [SMILES: [2H]C([2H])([2H])n1cc(-c2cc(OC)c(CN3CCN(CCC4CCN(c5ccc6c(c5)C(=O)N(C5CCC(=O)NC5=O)C6=O)CC4)CC3)c(OC)c2)c2c(c1=O)CN(C(=O)NCC(F)F)CC2] |
| F327 | 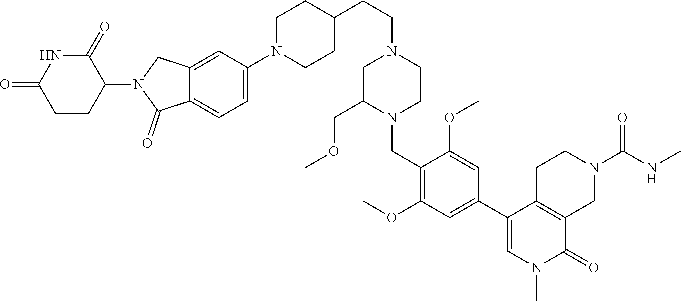 [SMILES: CNC(=O)N1CCc2c(-c3cc(OC)c(CN4CCN(CCC5CCN(c6ccc7c(c6)CN(C6CCC(=O)NC6=O)C7=O)CC5)CC4COC)c(OC)c3)cn(C)c(=O)c2C1] |
| F328 | 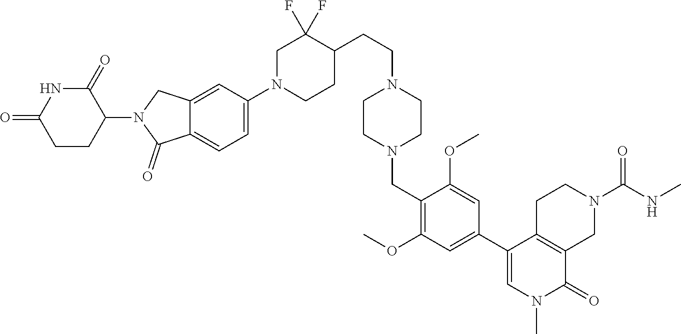 [SMILES: CNC(=O)N1CCc2c(-c3cc(OC)c(CN4CCN(CCC5CCN(c6ccc7c(c6)CN(C6CCC(=O)NC6=O)C7=O)CC5(F)F)CC4)c(OC)c3)cn(C)c(=O)c2C1] |
| F329 | 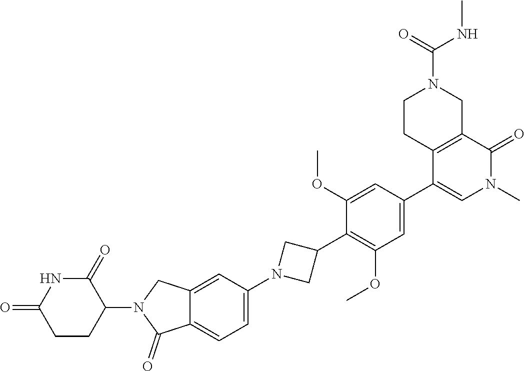 [SMILES: CNC(=O)N1CCc2c(-c3cc(OC)c(C4CN(c5ccc6c(c5)CN(C5CCC(=O)NC5=O)C6=O)C4)c(OC)c3)cn(C)c(=O)c2C1] |
| F330 | 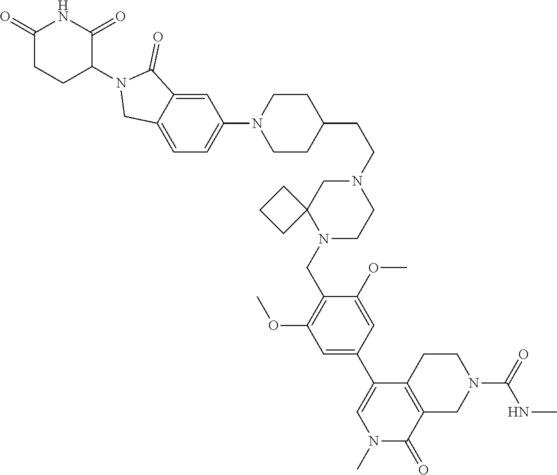 [SMILES: CNC(=O)N1CCc2c(-c3cc(OC)c(CN4CCN(CCC5CCN(c6ccc7c(c6)C(=O)N(C6CCC(=O)NC6=O)C7)CC5)CC45CCC5)c(OC)c3)cn(C)c(=O)c2C1] |
| F331 | 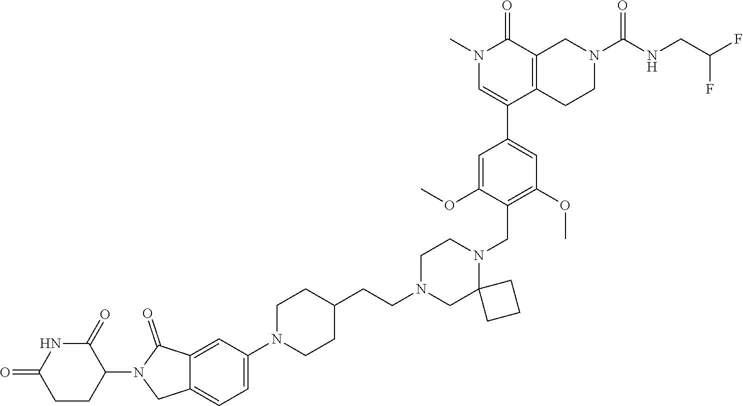 [SMILES: COc1cc(-c2cn(C)c(=O)c3c2CCN(C(=O)NCC(F)F)C3)cc(OC)c1CN1CCN(CCC2CCN(c3ccc4c(c3)C(=O)N(C3CCC(=O)NC3=O)C4)CC2)CC12CCC2] |
| F332 | 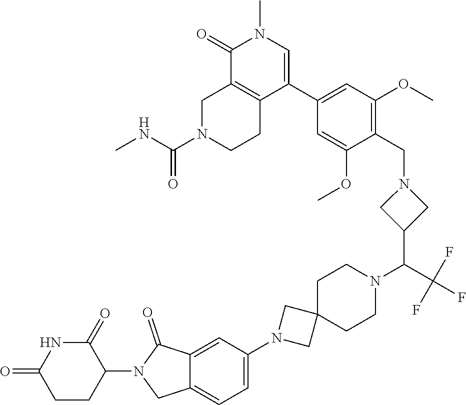 [SMILES: CNC(=O)N1CCc2c(-c3cc(OC)c(CN4CC(C(N5CCC6(CC5)CN(c5ccc7c(c5)C(=O)N(C5CCC(=O)NC5=O)C7)C6)C(F)(F)F)C4)c(OC)c3)cn(C)c(=O)c2C1] |
| F333 | 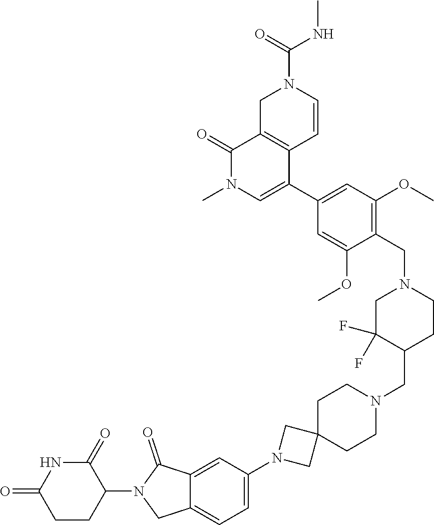 [SMILES: CNC(=O)N1C=Cc2c(-c3cc(OC)c(CN4CCC(CN5CCC6(CC5)CN(c5ccc7c(c5)C(=O)N(C5CCC(=O)NC5=O)C7)C6)C(F)(F)C4)c(OC)c3)cn(C)c(=O)c2C1] |
| F334 | 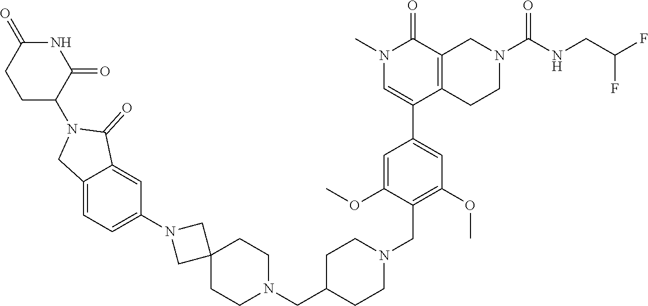 [SMILES: COc1cc(-c2cn(C)c(=O)c3c2CCN(C(=O)NCC(F)F)C3)cc(OC)c1CN1CCC(CN2CCC3(CC2)CN(c2ccc4c(c2)C(=O)N(C2CCC(=O)NC2=O)C4)C3)CC1] |
| F335 | 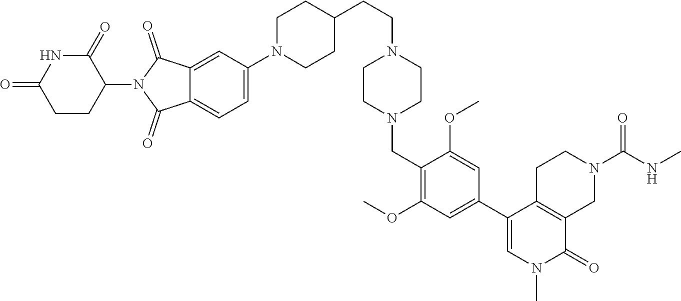 [SMILES: CNC(=O)N1CCc2c(-c3cc(OC)c(CN4CCN(CCC5CCN(c6ccc7c(c6)C(=O)N(C6CCC(=O)NC6=O)C7=O)CC5)CC4)c(OC)c3)cn(C)c(=O)c2C1] |
| F336 | 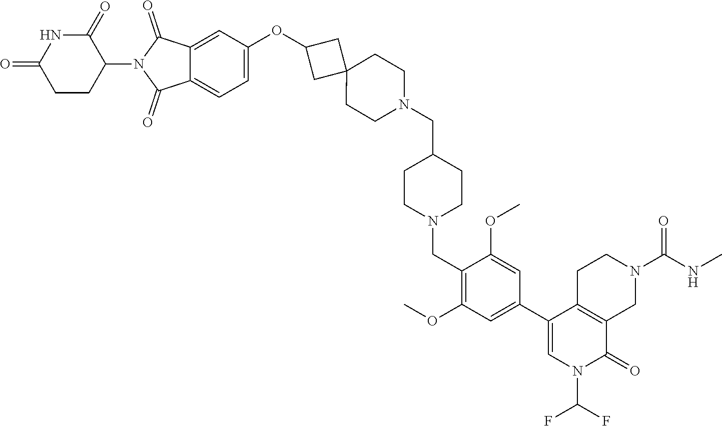 [SMILES: CNC(=O)N1CCc2c(-c3cc(OC)c(CN4CCC(CN5CCC6(CC5)CC(Oc5ccc7c(c5)C(=O)N(C5CCC(=O)NC5=O)C7=O)C6)CC4)c(OC)c3)cn(C(F)F)c(=O)c2C1] |
| F337 | 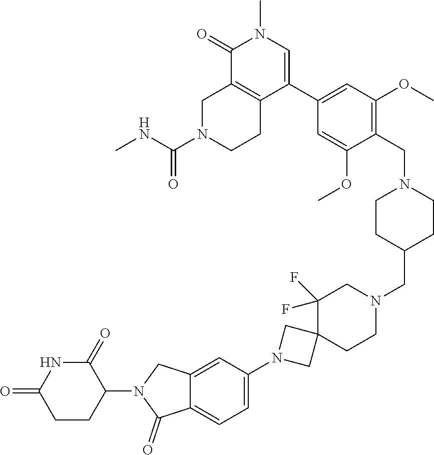 [SMILES: CNC(=O)N1CCc2c(-c3cc(OC)c(CN4CCC(CN5CCC6(CN(c7ccc8c(c7)CN(C7CCC(=O)NC7=O)C8=O)C6)C(F)(F)C5)CC4)c(OC)c3)cn(C)c(=O)c2C1] |
| F338 | 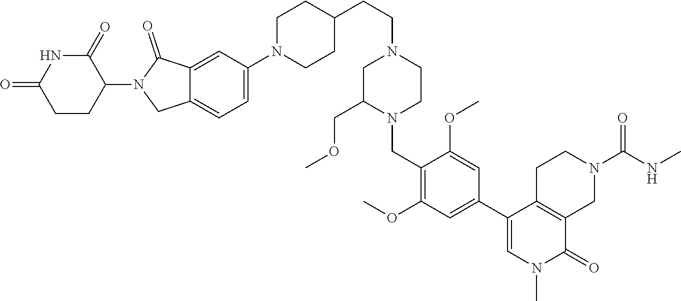 [SMILES: CNC(=O)N1CCc2c(-c3cc(OC)c(CN4CCN(CCC5CCN(c6ccc7c(c6)C(=O)N(C6CCC(=O)NC6=O)C7)CC5)CC4COC)c(OC)c3)cn(C)c(=O)c2C1] |
| F339 | 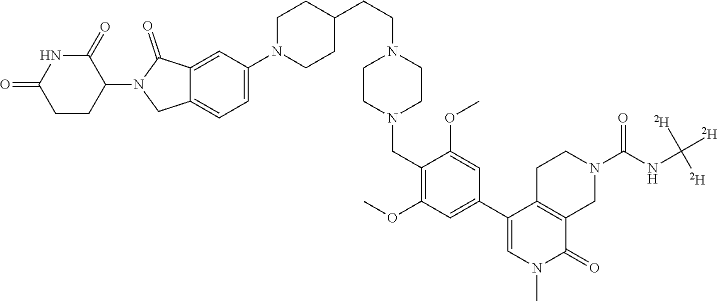 [SMILES: [2H]C([2H])([2H])NC(=O)N1CCc2c(-c3cc(OC)c(CN4CCN(CCC5CCN(c6ccc7c(c6)C(=O)N(C6CCC(=O)NC6=O)C7)CC5)CC4)c(OC)c3)cn(C)c(=O)c2C1] |
| F340 | 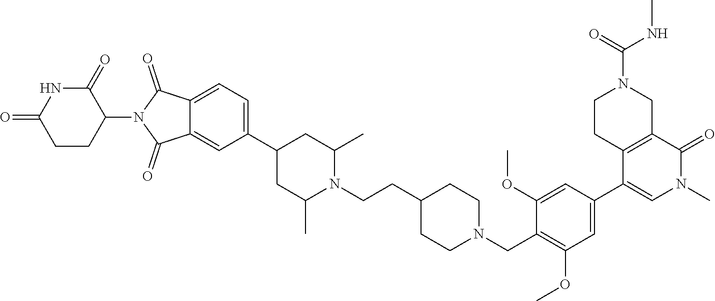 [SMILES: CNC(=O)N1CCc2c(-c3cc(OC)c(CN4CCC(CCN5C(C)CC(c6ccc7c(c6)C(=O)N(C6CCC(=O)NC6=O)C7=O)CC5C)CC4)c(OC)c3)cn(C)c(=O)c2C1] |
| F341 | 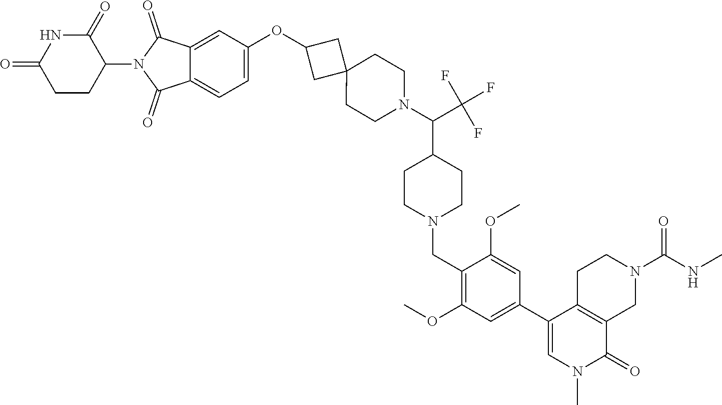 [SMILES: CNC(=O)N1CCc2c(-c3cc(OC)c(CN4CCC(C(N5CCC6(CC5)CC(Oc5ccc7c(c5)C(=O)N(C5CCC(=O)NC5=O)C7=O)C6)C(F)(F)F)CC4)c(OC)c3)cn(C)c(=O)c2C1] |
| F342 | 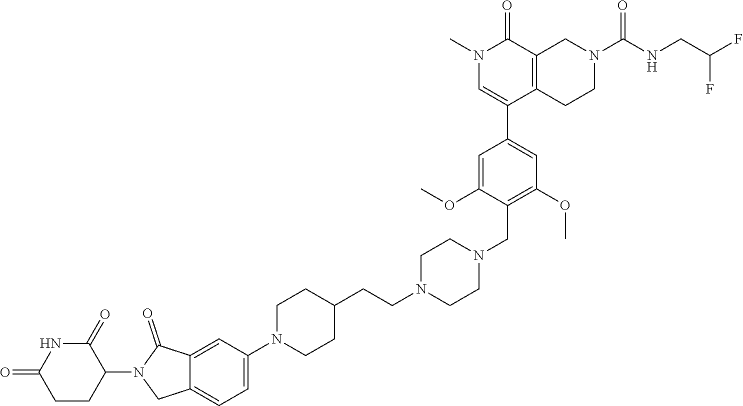 [SMILES: COc1cc(-c2cn(C)c(=O)c3c2CCN(C(=O)NCC(F)F)C3)cc(OC)c1CN1CCN(CCC2CCN(c3ccc4c(c3)C(=O)N(C3CCC(=O)NC3=O)C4)CC2)CC1] |
| F343 | 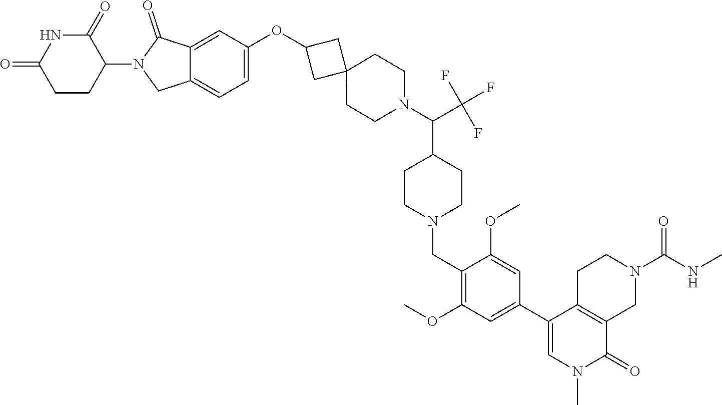 [SMILES: CNC(=O)N1CCc2c(-c3cc(OC)c(CN4CCC(C(N5CCC6(CC5)CC(Oc5ccc7c(c5)C(=O)N(C5CCC(=O)NC5=O)C7)C6)C(F)(F)F)CC4)c(OC)c3)cn(C)c(=O)c2C1] |
| F344 | 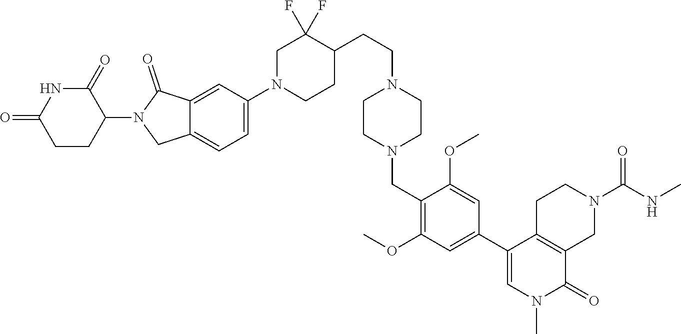 [SMILES: CNC(=O)N1CCc2c(-c3cc(OC)c(CN4CCN(CCC5CCN(c6ccc7c(c6)C(=O)N(C6CCC(=O)NC6=O)C7)CC5(F)F)CC4)c(OC)c3)cn(C)c(=O)c2C1] |
| F345 | 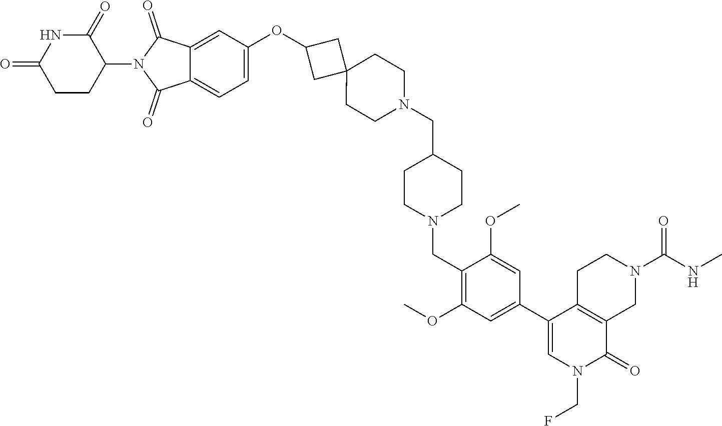 [SMILES: CNC(=O)N1CCc2c(-c3cc(OC)c(CN4CCC(CN5CCC6(CC5)CC(Oc5ccc7c(c5)C(=O)N(C5CCC(=O)NC5=O)C7=O)C6)CC4)c(OC)c3)cn(CF)c(=O)c2C1] |
| F346 | 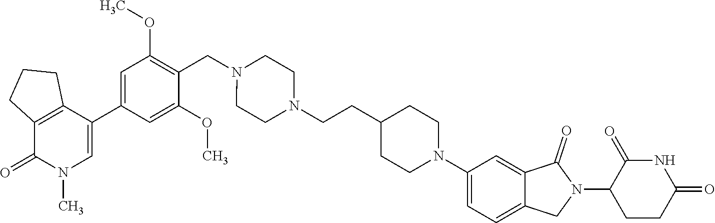 [SMILES: COc1cc(-c2cn(C)c(=O)c3c2CCC3)cc(OC)c1CN1CCN(CCC2CCN(c3ccc4c(c3)C(=O)N(C3CCC(=O)NC3=O)C4)CC2)CC1] |
| F347 | 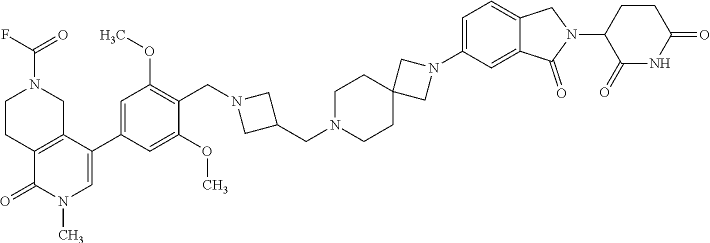 [SMILES: COc1cc(-c2cn(C)c(=O)c3c2CN(C(=O)F)CC3)cc(OC)c1CN1CC(CN2CCC3(CC2)CN(c2ccc4c(c2)C(=O)N(C2CCC(=O)NC2=O)C4)C3)C1] |
| F348 | 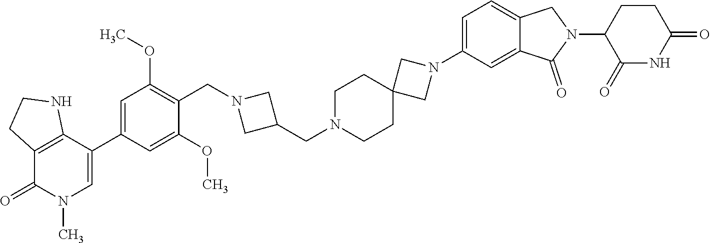 [SMILES: COc1cc(-c2cn(C)c(=O)c3c2NCC3)cc(OC)c1CN1CC(CN2CCC3(CC2)CN(c2ccc4c(c2)C(=O)N(C2CCC(=O)NC2=O)C4)C3)C1] |
| F349 | 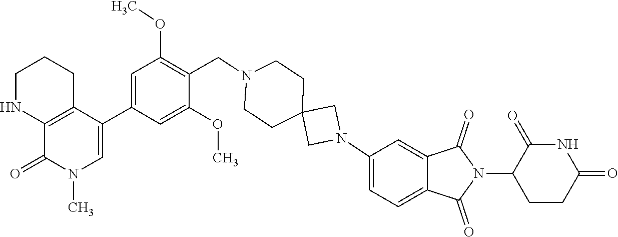 [SMILES: COc1cc(-c2cn(C)c(=O)c3c2CCCN3)cc(OC)c1CN1CCC2(CC1)CN(c1ccc3c(c1)C(=O)N(C1CCC(=O)NC1=O)C3=O)C2] |
| F350 | 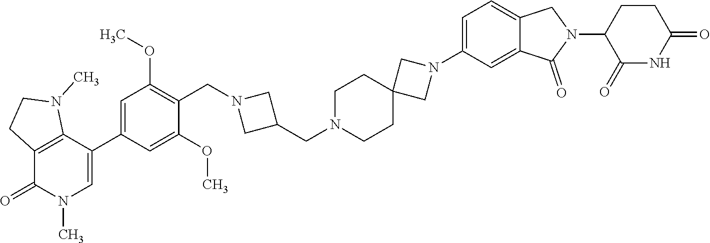 [SMILES: COc1cc(-c2cn(C)c(=O)c3c2N(C)CC3)cc(OC)c1CN1CC(CN2CCC3(CC2)CN(c2ccc4c(c2)C(=O)N(C2CCC(=O)NC2=O)C4)C3)C1] |
| F351 | 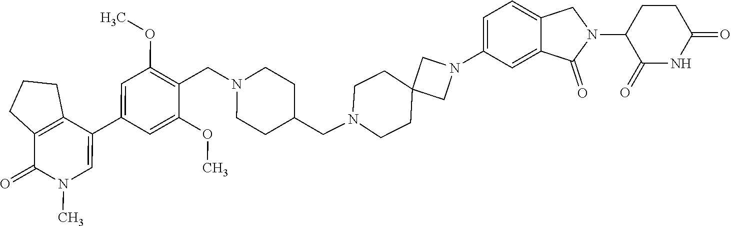 [SMILES: COc1cc(-c2cn(C)c(=O)c3c2CCC3)cc(OC)c1CN1CCC(CN2CCC3(CC2)CN(c2ccc4c(c2)C(=O)N(C2CCC(=O)NC2=O)C4)C3)CC1] |
| F352 | 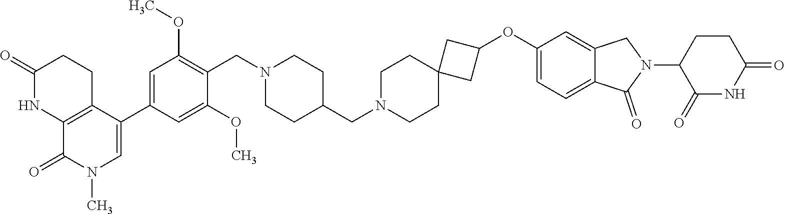 [SMILES: COc1cc(-c2cn(C)c(=O)c3c2CCC(=O)N3)cc(OC)c1CN1CCC(CN2CCC3(CC2)CC(Oc2ccc4c(c2)CN(C2CCC(=O)NC2=O)C4=O)C3)CC1] |
| F353 | 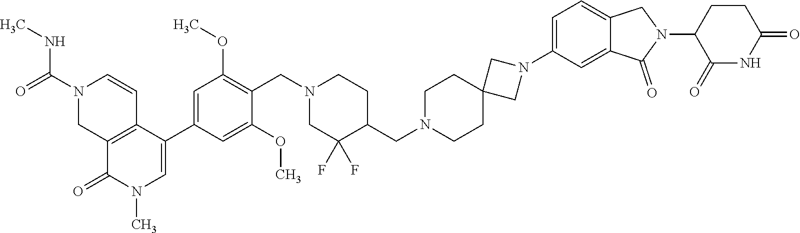 [SMILES: CNC(=O)N1C=Cc2c(-c3cc(OC)c(CN4CCC(CN5CCC6(CC5)CN(c5ccc7c(c5)C(=O)N(C5CCC(=O)NC5=O)C7)C6)C(F)(F)C4)c(OC)c3)cn(C)c(=O)c2C1] |
| F354 | 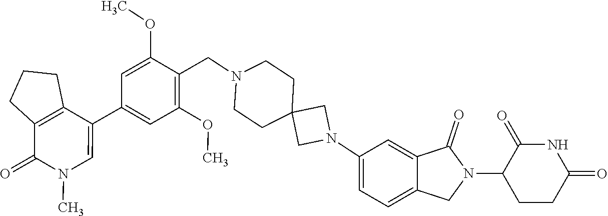 [SMILES: COc1cc(-c2cn(C)c(=O)c3c2CCC3)cc(OC)c1CN1CCC2(CC1)CN(c1ccc3c(c1)C(=O)N(C1CCC(=O)NC1=O)C3)C2] |
| F355 | 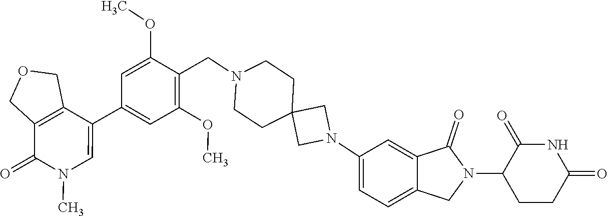 [SMILES: COc1cc(-c2cn(C)c(=O)c3c2COC3)cc(OC)c1CN1CCC2(CC1)CN(c1ccc3c(c1)C(=O)N(C1CCC(=O)NC1=O)C3)C2] |
| F356 | 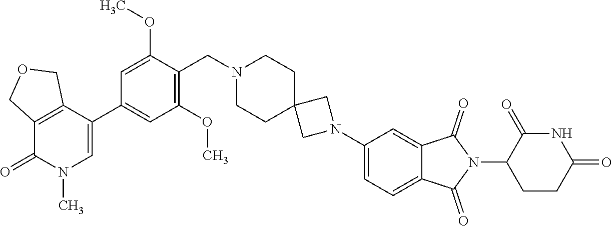 [SMILES: COc1cc(-c2cn(C)c(=O)c3c2COC3)cc(OC)c1CN1CCC2(CC1)CN(c1ccc3c(c1)C(=O)N(C1CCC(=O)NC1=O)C3=O)C2] |
| F357 | 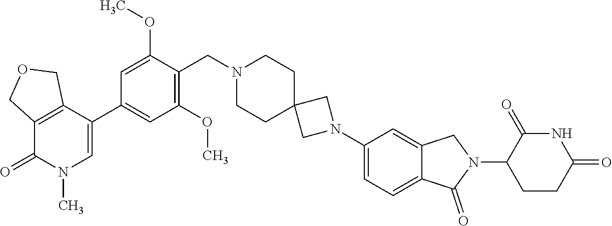 [SMILES: COc1cc(-c2cn(C)c(=O)c3c2COC3)cc(OC)c1CN1CCC2(CC1)CN(c1ccc3c(c1)CN(C1CCC(=O)NC1=O)C3=O)C2] |
| F358 | 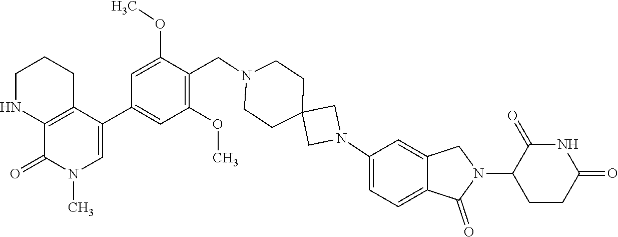 [SMILES: COc1cc(-c2cn(C)c(=O)c3c2CCCN3)cc(OC)c1CN1CCC2(CC1)CN(c1ccc3c(c1)CN(C1CCC(=O)NC1=O)C3=O)C2] |
| F359 | 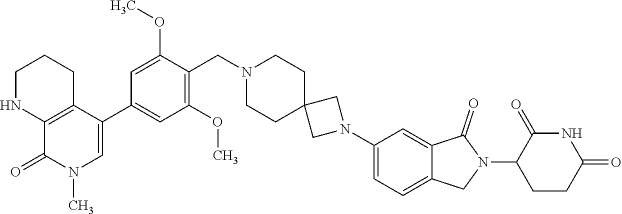 [SMILES: COc1cc(-c2cn(C)c(=O)c3c2CCCN3)cc(OC)c1CN1CCC2(CC1)CN(c1ccc3c(c1)C(=O)N(C1CCC(=O)NC1=O)C3)C2] |
| F360 | 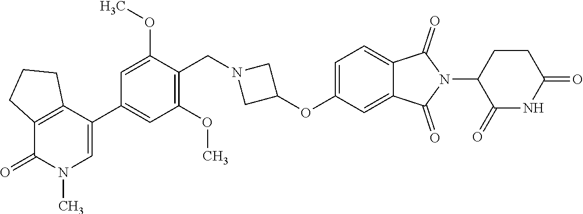 [SMILES: COc1cc(-c2cn(C)c(=O)c3c2CCC3)cc(OC)c1CN1CC(Oc2ccc3c(c2)C(=O)N(C2CCC(=O)NC2=O)C3=O)C1] |
| F361 | 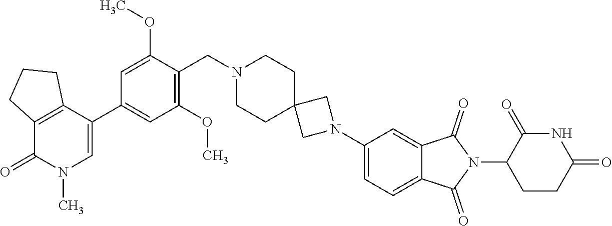 [SMILES: COc1cc(-c2cn(C)c(=O)c3c2CCC3)cc(OC)c1CN1CCC2(CC1)CN(c1ccc3c(c1)C(=O)N(C1CCC(=O)NC1=O)C3=O)C2] |
| F362 | 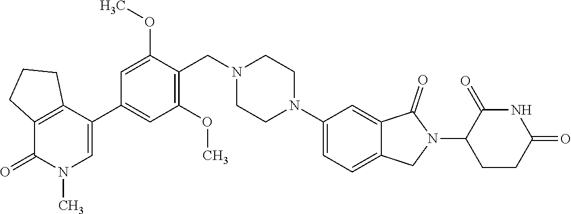 [SMILES: COc1cc(-c2cn(C)c(=O)c3c2CCC3)cc(OC)c1CN1CCN(c2ccc3c(c2)C(=O)N(C2CCC(=O)NC2=O)C3)CC1] |
| F363 | 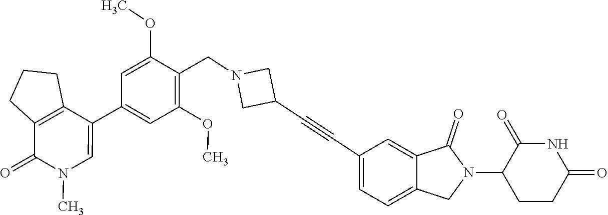 [SMILES: COc1cc(-c2cn(C)c(=O)c3c2CCC3)cc(OC)c1CN1CC(C#Cc2ccc3c(c2)C(=O)N(C2CCC(=O)NC2=O)C3)C1] |
| F364 | 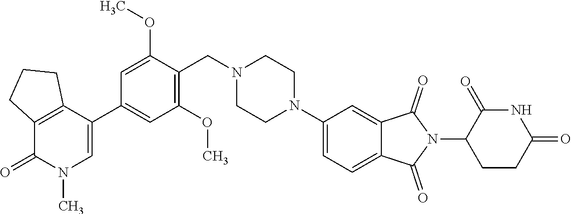 [SMILES: COc1cc(-c2cn(C)c(=O)c3c2CCC3)cc(OC)c1CN1CCN(c2ccc3c(c2)C(=O)N(C2CCC(=O)NC2=O)C3=O)CC1] |
| F365 | 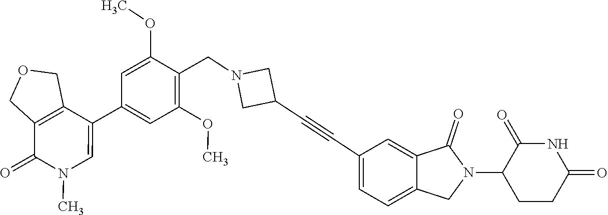 [SMILES: COc1cc(-c2cn(C)c(=O)c3c2COC3)cc(OC)c1CN1CC(C#Cc2ccc3c(c2)C(=O)N(C2CCC(=O)NC2=O)C3)C1] |
| F366 | 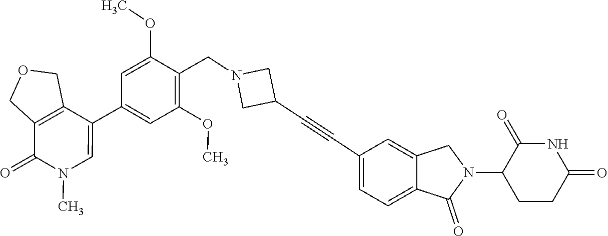 [SMILES: COc1cc(-c2cn(C)c(=O)c3c2COC3)cc(OC)c1CN1CC(C#Cc2ccc3c(c2)CN(C2CCC(=O)NC2=O)C3=O)C1] |
| F367 | 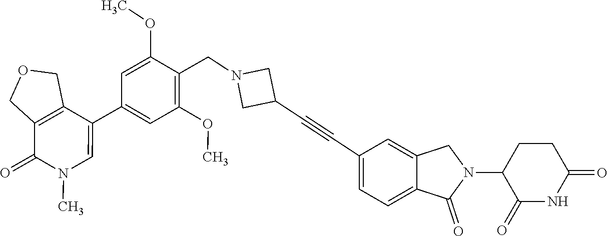 [SMILES: COc1cc(-c2cn(C)c(=O)c3c2COC3)cc(OC)c1CN1CC(C#Cc2ccc3c(c2)CN(C2CCC(=O)NC2=O)C3=O)C1] |
| F368 | 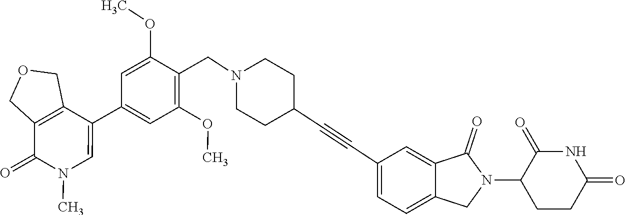 [SMILES: COc1cc(-c2cn(C)c(=O)c3c2COC3)cc(OC)c1CN1CCC(C#Cc2ccc3c(c2)C(=O)N(C2CCC(=O)NC2=O)C3)CC1] |
| F369 | 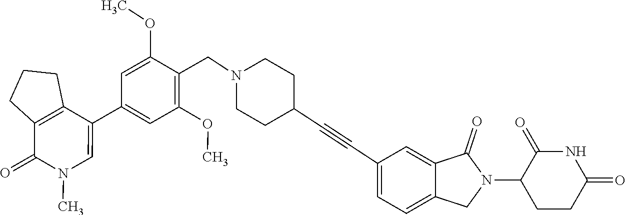 [SMILES: COc1cc(-c2cn(C)c(=O)c3c2CCC3)cc(OC)c1CN1CCC(C#Cc2ccc3c(c2)C(=O)N(C2CCC(=O)NC2=O)C3)CC1] |
| F370 | 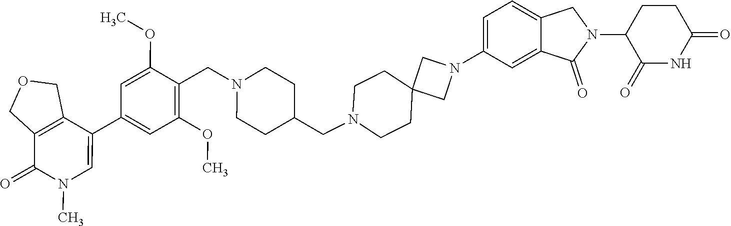 [SMILES: COc1cc(-c2cn(C)c(=O)c3c2COC3)cc(OC)c1CN1CCC(CN2CCC3(CC2)CN(c2ccc4c(c2)C(=O)N(C2CCC(=O)NC2=O)C4)C3)CC1] |
| F371 | 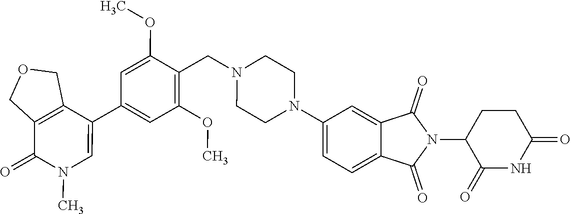 [SMILES: COc1cc(-c2cn(C)c(=O)c3c2COC3)cc(OC)c1CN1CCN(c2ccc3c(c2)C(=O)N(C2CCC(=O)NC2=O)C3=O)CC1] |
| F372 | 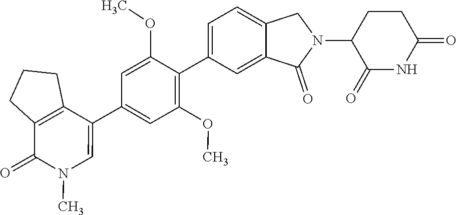 [SMILES: COc1cc(-c2cn(C)c(=O)c3c2CCC3)cc(OC)c1-c1ccc2c(c1)C(=O)N(C1CCC(=O)NC1=O)C2] |
| F373 | 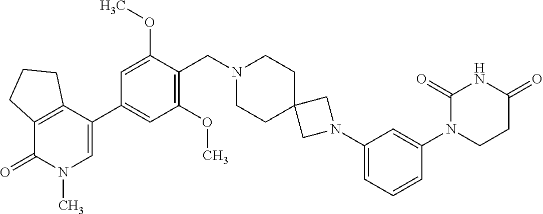 [SMILES: COc1cc(-c2cn(C)c(=O)c3c2CCC3)cc(OC)c1CN1CCC2(CC1)CN(c1cccc(N3CCC(=O)NC3=O)c1)C2] |

or a pharmaceutically acceptable salt thereof.

The BRD9 inhibitor may be, e.g., a compound selected from the group consisting of:

| Compound | |
|---|---|
| No. | Structure |
| G1 | 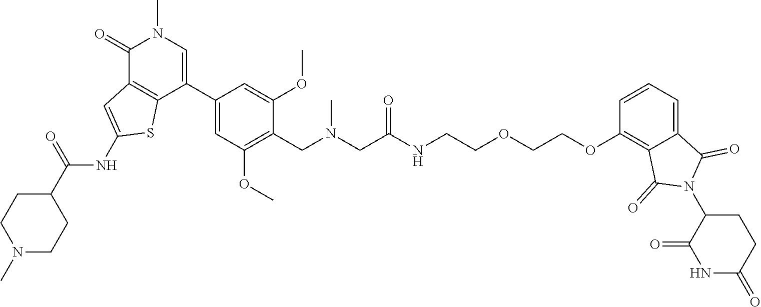 [SMILES: COc1cc(-c2cn(C)c(=O)c3cc(NC(=O)C4CCN(C)CC4)sc23)cc(OC)c1CN(C)CC(=O)NCCOCCOc1cccc2c1C(=O)N(C1CCC(=O)NC1=O)C2=O] |
| G2 | 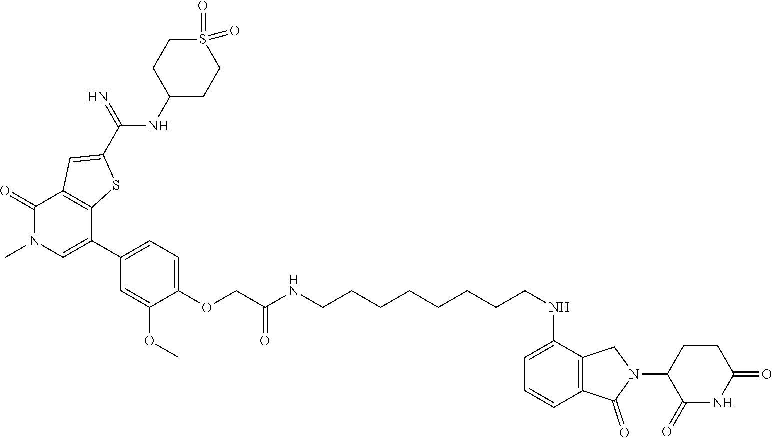 [SMILES: COc1cc(-c2cn(C)c(=O)c3cc(C(=N)NC4CCS(=O)(=O)CC4)sc23)ccc1OCC(=O)NCCCCCCCCNc1cccc2c1CN(C1CCC(=O)NC1=O)C2=O] |
| G3 | 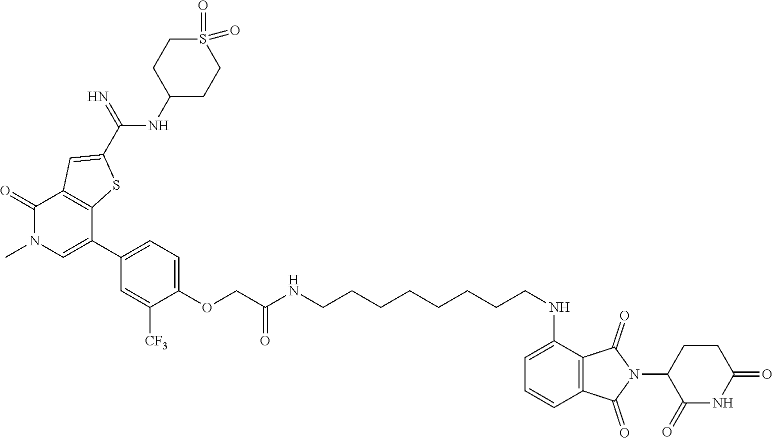 [SMILES: Cn1cc(-c2ccc(OCC(=O)NCCCCCCCCNc3cccc4c3C(=O)N(C3CCC(=O)NC3=O)C4=O)c(C(F)(F)F)c2)c2sc(C(=N)NC3CCS(=O)(=O)CC3)cc2c1=O] |
| G4 | 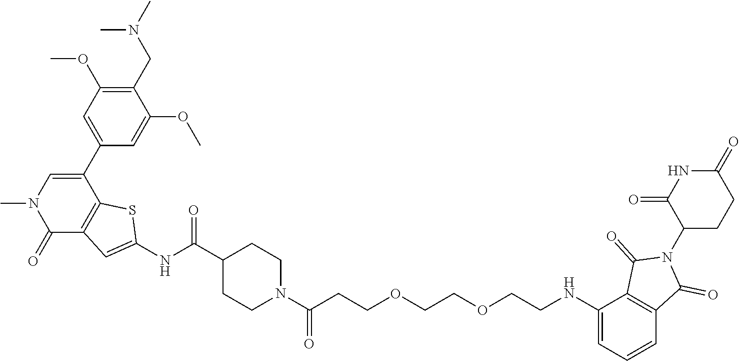 [SMILES: COc1cc(-c2cn(C)c(=O)c3cc(NC(=O)C4CCN(C(=O)CCOCCOCCNc5cccc6c5C(=O)N(C5CCC(=O)NC5=O)C6=O)CC4)sc23)cc(OC)c1CN(C)C] |
| G5 | 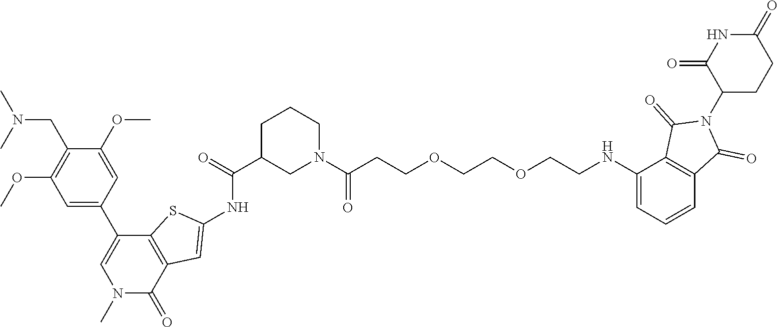 [SMILES: COc1cc(-c2cn(C)c(=O)c3cc(NC(=O)C4CCCN(C(=O)CCOCCOCCNc5cccc6c5C(=O)N(C5CCC(=O)NC5=O)C6=O)C4)sc23)cc(OC)c1CN(C)C] |
| G6 | 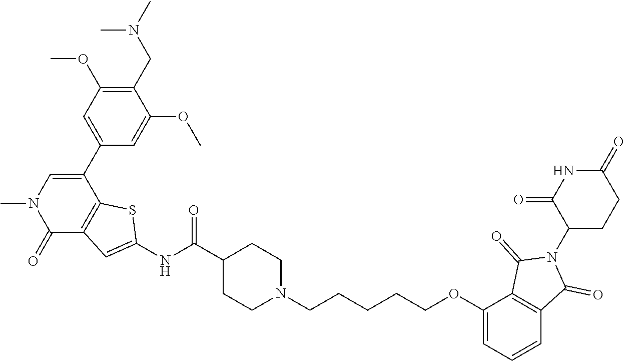 [SMILES: COc1cc(-c2cn(C)c(=O)c3cc(NC(=O)C4CCN(CCCCCOc5cccc6c5C(=O)N(C5CCC(=O)NC5=O)C6=O)CC4)sc23)cc(OC)c1CN(C)C] |
| G7 | 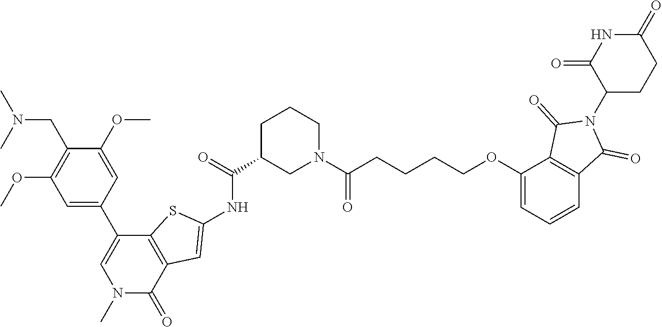 [SMILES: COc1cc(-c2cn(C)c(=O)c3cc(NC(=O)[C@@H]4CCCN(C(=O)CCCCOc5cccc6c5C(=O)N(C5CCC(=O)NC5=O)C6=O)C4)sc23)cc(OC)c1CN(C)C] |
| G8 | 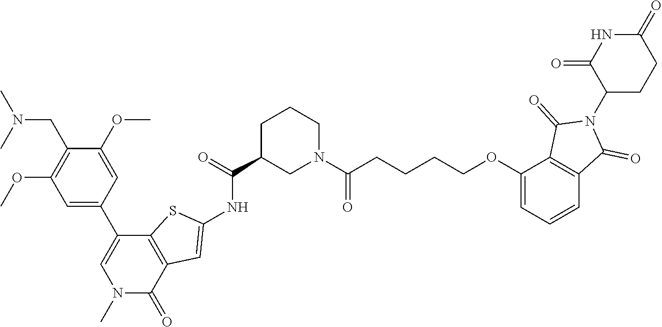 [SMILES: COc1cc(-c2cn(C)c(=O)c3cc(NC(=O)[C@H]4CCCN(C(=O)CCCCOc5cccc6c5C(=O)N(C5CCC(=O)NC5=O)C6=O)C4)sc23)cc(OC)c1CN(C)C] |
| G9 | 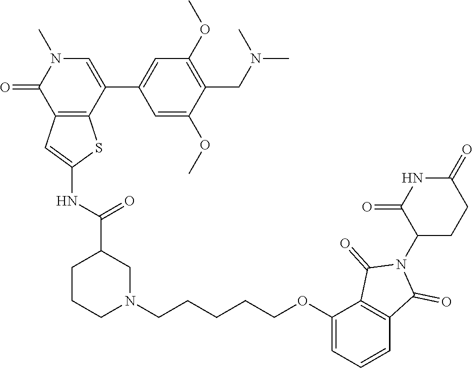 [SMILES: COc1cc(-c2cn(C)c(=O)c3cc(NC(=O)C4CCCN(CCCCCOc5cccc6c5C(=O)N(C5CCC(=O)NC5=O)C6=O)C4)sc23)cc(OC)c1CN(C)C] |
| G10 | 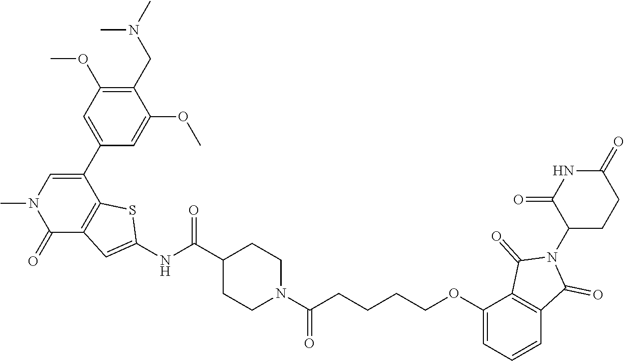 [SMILES: COc1cc(-c2cn(C)c(=O)c3cc(NC(=O)C4CCN(C(=O)CCCCOc5cccc6c5C(=O)N(C5CCC(=O)NC5=O)C6=O)CC4)sc23)cc(OC)c1CN(C)C] |
| G11 | 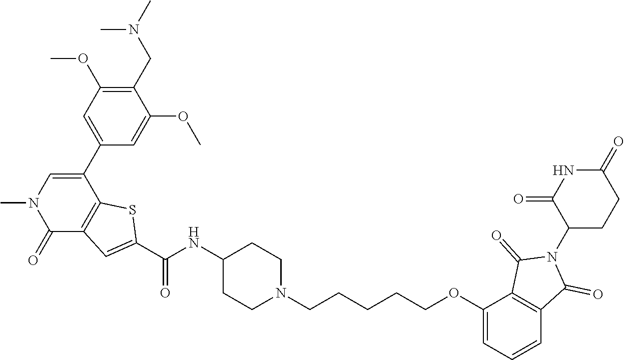 [SMILES: COc1cc(-c2cn(C)c(=O)c3cc(C(=O)NC4CCN(CCCCCOc5cccc6c5C(=O)N(C5CCC(=O)NC5=O)C6=O)CC4)sc23)cc(OC)c1CN(C)C] |
| G12 | 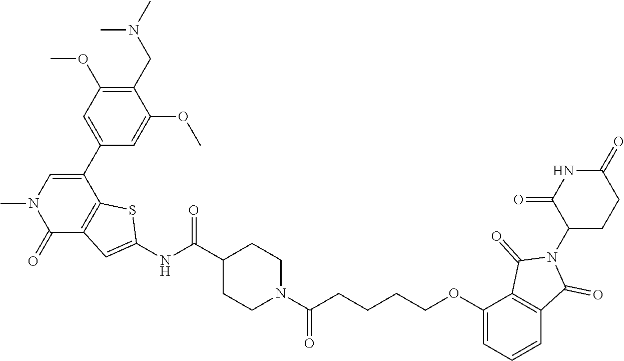 [SMILES: COc1cc(-c2cn(C)c(=O)c3cc(NC(=O)C4CCN(C(=O)CCCCOc5cccc6c5C(=O)N(C5CCC(=O)NC5=O)C6=O)CC4)sc23)cc(OC)c1CN(C)C] |
| G13 | 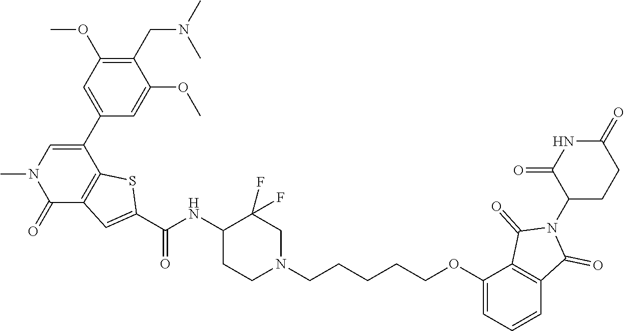 [SMILES: COc1cc(-c2cn(C)c(=O)c3cc(C(=O)NC4CCN(CCCCCOc5cccc6c5C(=O)N(C5CCC(=O)NC5=O)C6=O)CC4(F)F)sc23)cc(OC)c1CN(C)C] |
| G14 | 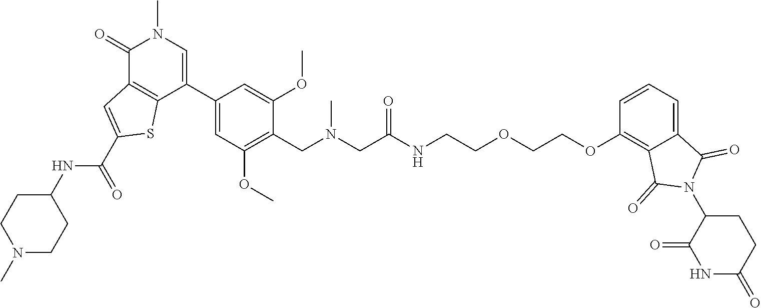 [SMILES: COc1cc(-c2cn(C)c(=O)c3cc(C(=O)NC4CCN(C)CC4)sc23)cc(OC)c1CN(C)CC(=O)NCCOCCOc1cccc2c1C(=O)N(C1CCC(=O)NC1=O)C2=O] |
| G15 | 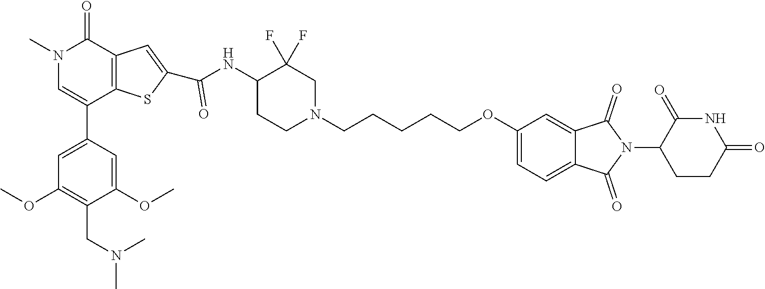 [SMILES: COc1cc(-c2cn(C)c(=O)c3cc(C(=O)NC4CCN(CCCCCOc5ccc6c(c5)C(=O)N(C5CCC(=O)NC5=O)C6=O)CC4(F)F)sc23)cc(OC)c1CN(C)C] |
| G16 | 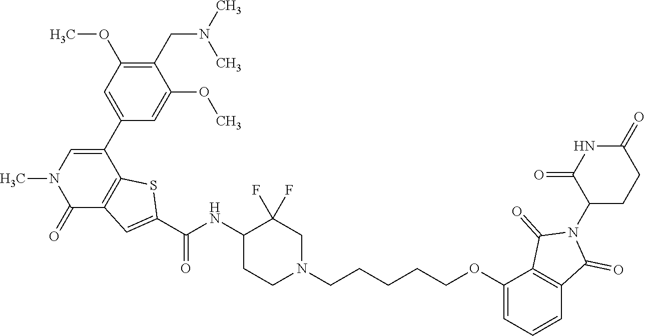 [SMILES: COc1cc(-c2cn(C)c(=O)c3cc(C(=O)NC4CCN(CCCCCOc5cccc6c5C(=O)N(C5CCC(=O)NC5=O)C6=O)CC4(F)F)sc23)cc(OC)c1CN(C)C] |
| G17 | 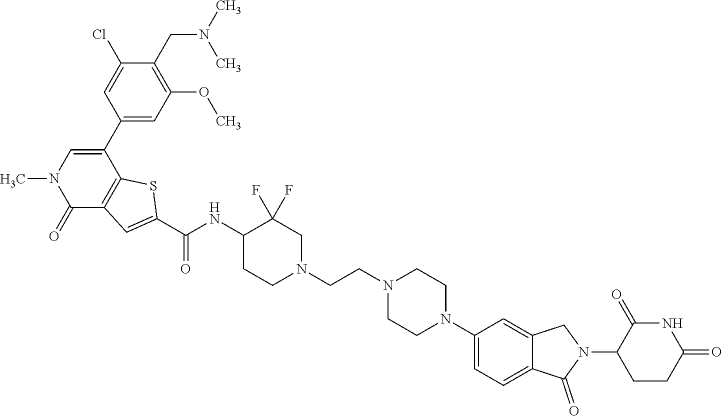 [SMILES: COc1cc(-c2cn(C)c(=O)c3cc(C(=O)NC4CCN(CCN5CCN(c6ccc7c(c6)CN(C6CCC(=O)NC6=O)C7=O)CC5)CC4(F)F)sc23)cc(Cl)c1CN(C)C] |
| G18 | 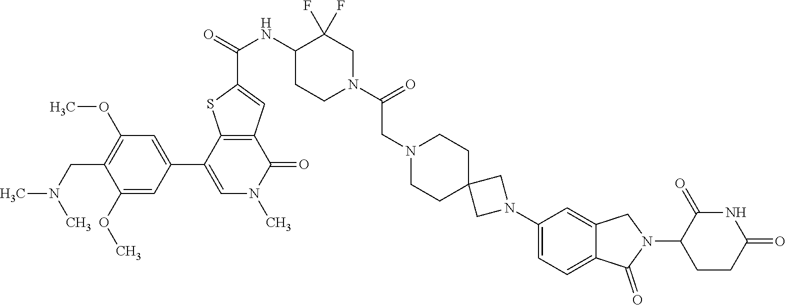 [SMILES: COc1cc(-c2cn(C)c(=O)c3cc(C(=O)NC4CCN(C(=O)CN5CCC6(CC5)CN(c5ccc7c(c5)CN(C5CCC(=O)NC5=O)C7=O)C6)CC4(F)F)sc23)cc(OC)c1CN(C)C] |
| G19 | 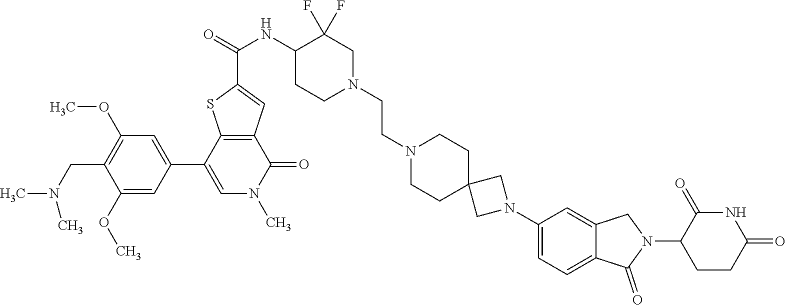 [SMILES: COc1cc(-c2cn(C)c(=O)c3cc(C(=O)NC4CCN(CCN5CCC6(CC5)CN(c5ccc7c(c5)CN(C5CCC(=O)NC5=O)C7=O)C6)CC4(F)F)sc23)cc(OC)c1CN(C)C] |
| G20 | 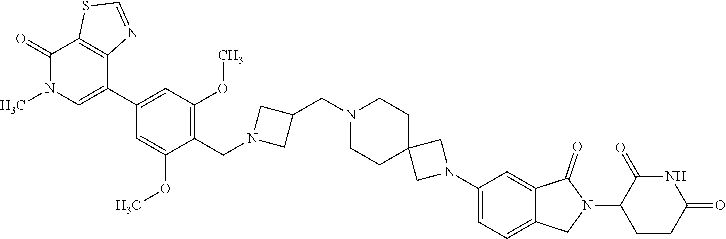 [SMILES: COc1cc(-c2cn(C)c(=O)c3scnc23)cc(OC)c1CN1CC(CN2CCC3(CC2)CN(c2ccc4c(c2)C(=O)N(C2CCC(=O)NC2=O)C4)C3)C1] |
| G21 | 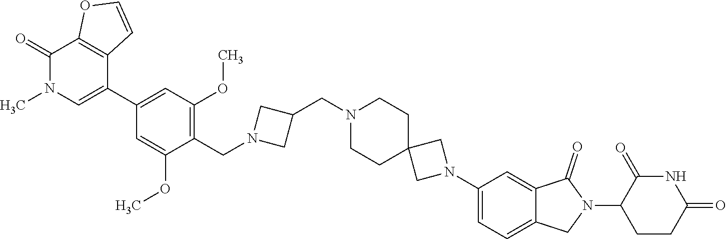 [SMILES: COc1cc(-c2cn(C)c(=O)c3occc23)cc(OC)c1CN1CC(CN2CCC3(CC2)CN(c2ccc4c(c2)C(=O)N(C2CCC(=O)NC2=O)C4)C3)C1] |
| G22 | 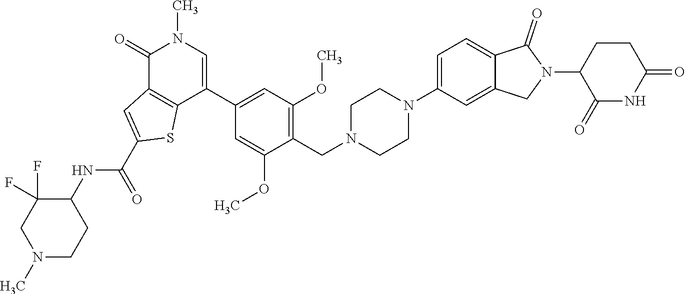 [SMILES: COc1cc(-c2cn(C)c(=O)c3cc(C(=O)NC4CCN(C)CC4(F)F)sc23)cc(OC)c1CN1CCN(c2ccc3c(c2)CN(C2CCC(=O)NC2=O)C3=O)CC1] |
| G23 | 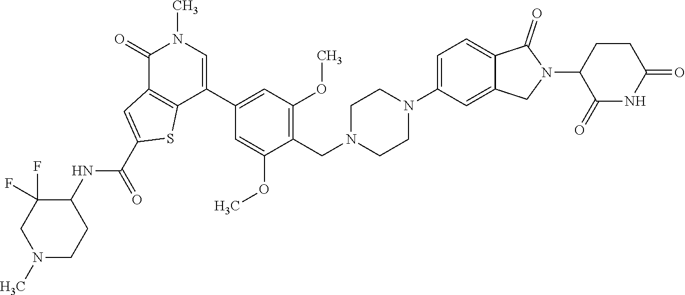 [SMILES: COc1cc(-c2cn(C)c(=O)c3cc(C(=O)NC4CCN(C)CC4(F)F)sc23)cc(OC)c1CN1CCN(c2ccc3c(c2)CN(C2CCC(=O)NC2=O)C3=O)CC1] |
| G24 | 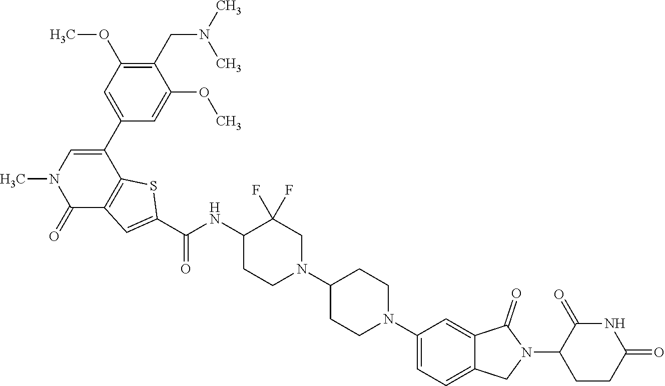 [SMILES: COc1cc(-c2cn(C)c(=O)c3cc(C(=O)NC4CCN(C5CCN(c6ccc7c(c6)C(=O)N(C6CCC(=O)NC6=O)C7)CC5)CC4(F)F)sc23)cc(OC)c1CN(C)C] |
| G25 | 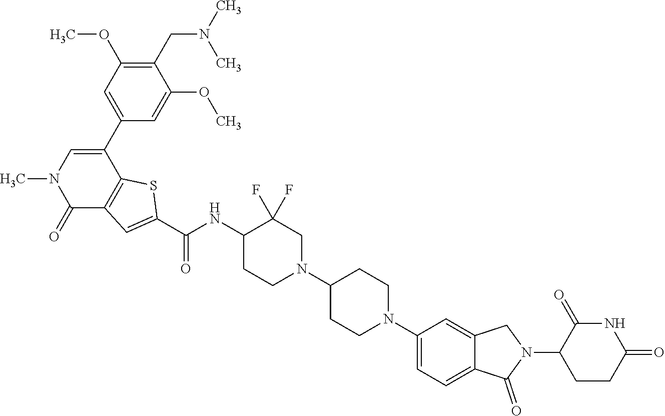 [SMILES: COc1cc(-c2cn(C)c(=O)c3cc(C(=O)NC4CCN(C5CCN(c6ccc7c(c6)CN(C6CCC(=O)NC6=O)C7=O)CC5)CC4(F)F)sc23)cc(OC)c1CN(C)C] |
| G26 | 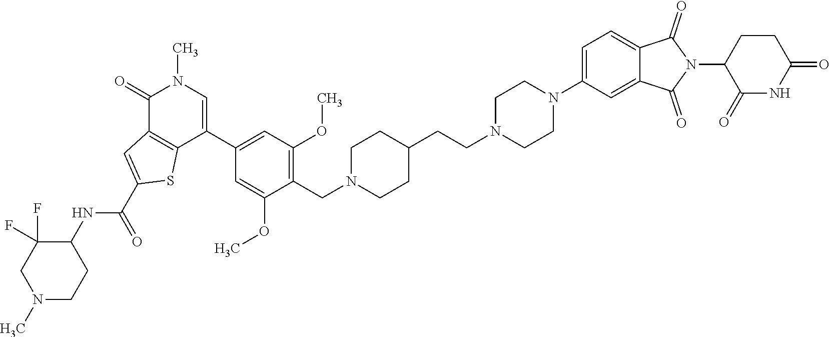 [SMILES: COc1cc(-c2cn(C)c(=O)c3cc(C(=O)NC4CCN(C)CC4(F)F)sc23)cc(OC)c1CN1CCC(CCN2CCN(c3ccc4c(c3)C(=O)N(C3CCC(=O)NC3=O)C4=O)CC2)CC1] |
| G27 | 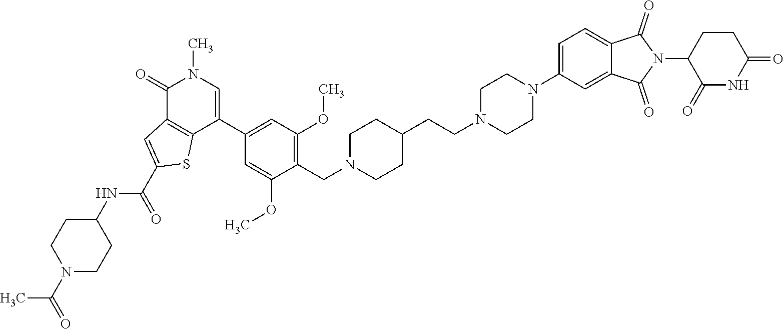 [SMILES: COc1cc(-c2cn(C)c(=O)c3cc(C(=O)NC4CCN(C(C)=O)CC4)sc23)cc(OC)c1CN1CCC(CCN2CCN(c3ccc4c(c3)C(=O)N(C3CCC(=O)NC3=O)C4=O)CC2)CC1] |
| G28 | 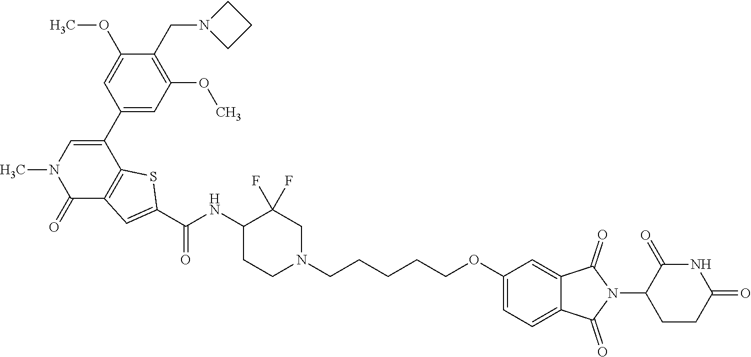 [SMILES: COc1cc(-c2cn(C)c(=O)c3cc(C(=O)NC4CCN(CCCCCOc5ccc6c(c5)C(=O)N(C5CCC(=O)NC5=O)C6=O)CC4(F)F)sc23)cc(OC)c1CN1CCC1] |
| G29 | 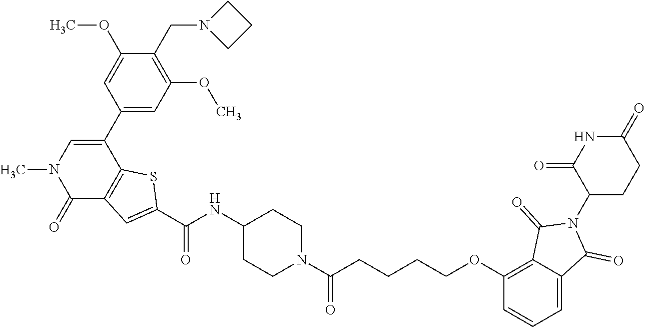 [SMILES: COc1cc(-c2cn(C)c(=O)c3cc(C(=O)NC4CCN(C(=O)CCCCOc5cccc6c5C(=O)N(C5CCC(=O)NC5=O)C6=O)CC4)sc23)cc(OC)c1CN1CCC1] |
| G30 | 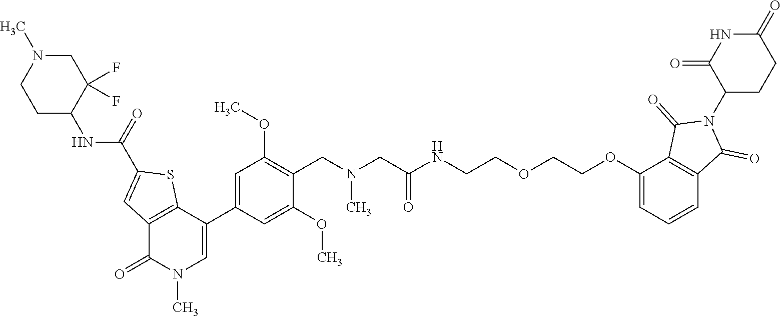 [SMILES: COc1cc(-c2cn(C)c(=O)c3cc(C(=O)NC4CCN(C)CC4(F)F)sc23)cc(OC)c1CN(C)CC(=O)NCCOCCOc1cccc2c1C(=O)N(C1CCC(=O)NC1=O)C2=O] |
| G31 | 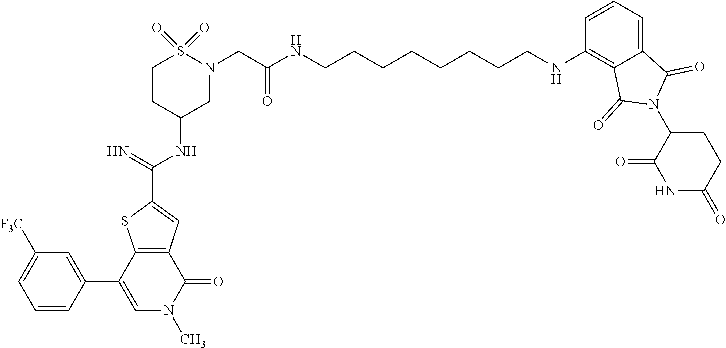 [SMILES: Cn1cc(-c2cccc(C(F)(F)F)c2)c2sc(C(=N)NC3CCS(=O)(=O)N(CC(=O)NCCCCCCCCNc4cccc5c4C(=O)N(C4CCC(=O)NC4=O)C5=O)C3)cc2c1=O] |
| G32 | 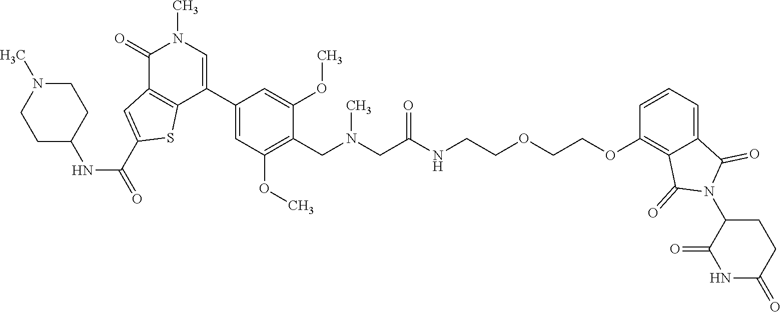 [SMILES: COc1cc(-c2cn(C)c(=O)c3cc(C(=O)NC4CCN(C)CC4)sc23)cc(OC)c1CN(C)CC(=O)NCCOCCOc1cccc2c1C(=O)N(C1CCC(=O)NC1=O)C2=O] |
| G33 | 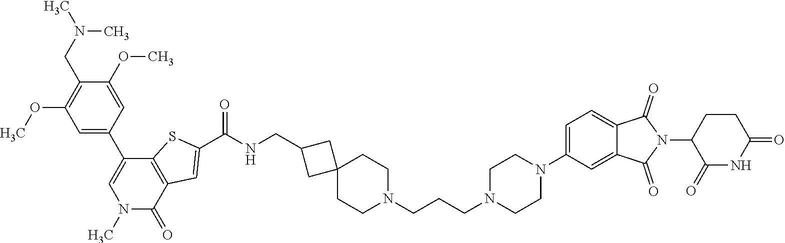 [SMILES: COc1cc(-c2cn(C)c(=O)c3cc(C(=O)NCC4CC5(CCN(CCCN6CCN(c7ccc8c(c7)C(=O)N(C7CCC(=O)NC7=O)C8=O)CC6)CC5)C4)sc23)cc(OC)c1CN(C)C] |
| G34 | 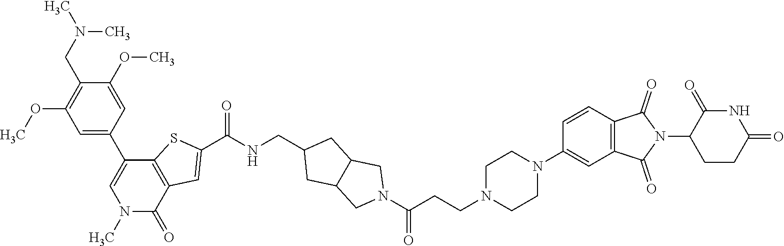 [SMILES: COc1cc(-c2cn(C)c(=O)c3cc(C(=O)NCC4CC5CN(C(=O)CCN6CCN(c7ccc8c(c7)C(=O)N(C7CCC(=O)NC7=O)C8=O)CC6)CC5C4)sc23)cc(OC)c1CN(C)C] |
| G35 | 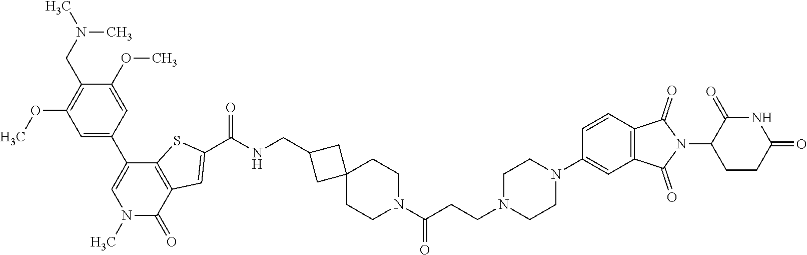 [SMILES: COc1cc(-c2cn(C)c(=O)c3cc(C(=O)NCC4CC5(CCN(C(=O)CCN6CCN(c7ccc8c(c7)C(=O)N(C7CCC(=O)NC7=O)C8=O)CC6)CC5)C4)sc23)cc(OC)c1CN(C)C] |
| G36 | 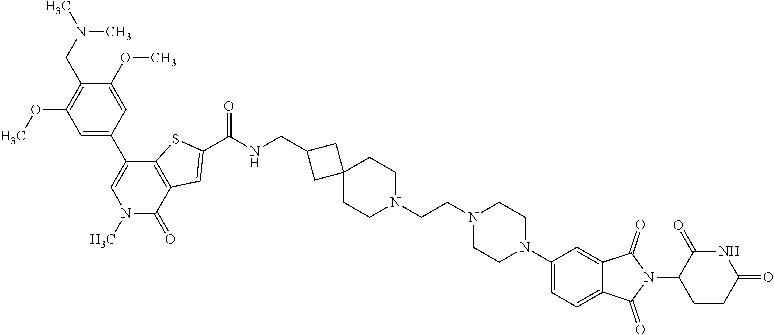 [SMILES: COc1cc(-c2cn(C)c(=O)c3cc(C(=O)NCC4CC5(CCN(CCN6CCN(c7ccc8c(c7)C(=O)N(C7CCC(=O)NC7=O)C8=O)CC6)CC5)C4)sc23)cc(OC)c1CN(C)C] |
| G37 | 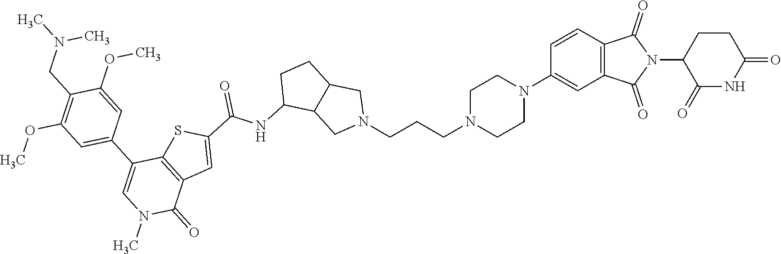 [SMILES: COc1cc(-c2cn(C)c(=O)c3cc(C(=O)NC4CCC5CN(CCCN6CCN(c7ccc8c(c7)C(=O)N(C7CCC(=O)NC7=O)C8=O)CC6)CC54)sc23)cc(OC)c1CN(C)C] |
| G38 | 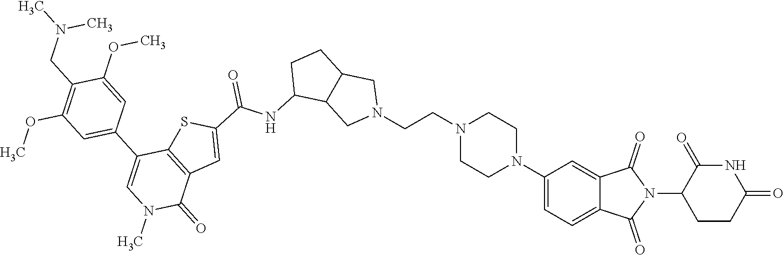 [SMILES: COc1cc(-c2cn(C)c(=O)c3cc(C(=O)NC4CCC5CN(CCN6CCN(c7ccc8c(c7)C(=O)N(C7CCC(=O)NC7=O)C8=O)CC6)CC54)sc23)cc(OC)c1CN(C)C] |
| G39 | 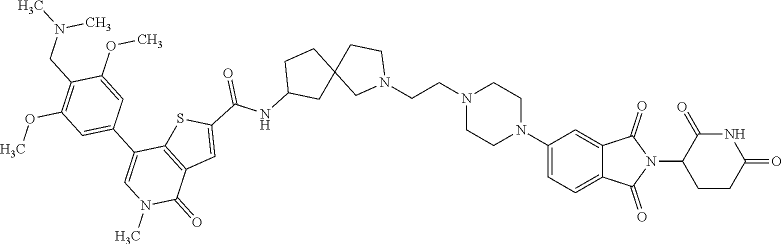 [SMILES: COc1cc(-c2cn(C)c(=O)c3cc(C(=O)NC4CCC5(CCN(CCN6CCN(c7ccc8c(c7)C(=O)N(C7CCC(=O)NC7=O)C8=O)CC6)C5)C4)sc23)cc(OC)c1CN(C)C] |
| G40 | 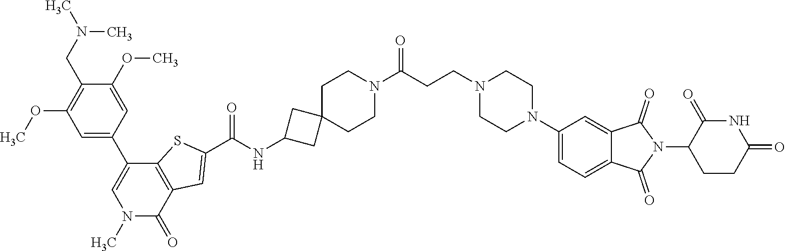 [SMILES: COc1cc(-c2cn(C)c(=O)c3cc(C(=O)NC4CC5(CCN(C(=O)CCN6CCN(c7ccc8c(c7)C(=O)N(C7CCC(=O)NC7=O)C8=O)CC6)CC5)C4)sc23)cc(OC)c1CN(C)C] |
| G41 | 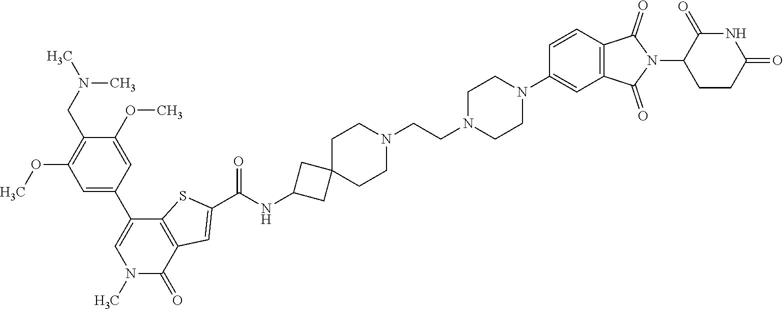 [SMILES: COc1cc(-c2cn(C)c(=O)c3cc(C(=O)NC4CC5(CCN(CCN6CCN(c7ccc8c(c7)C(=O)N(C7CCC(=O)NC7=O)C8=O)CC6)CC5)C4)sc23)cc(OC)c1CN(C)C] |
| G42 | 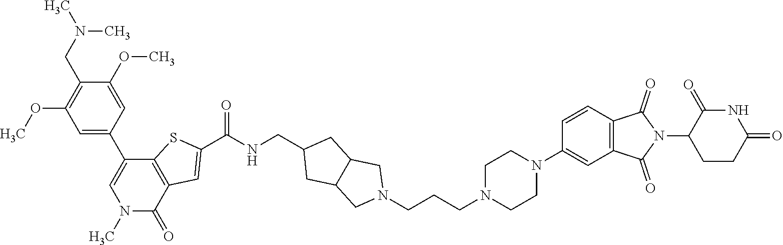 [SMILES: COc1cc(-c2cn(C)c(=O)c3cc(C(=O)NCC4CC5CN(CCCN6CCN(c7ccc8c(c7)C(=O)N(C7CCC(=O)NC7=O)C8=O)CC6)CC5C4)sc23)cc(OC)c1CN(C)C] |
| G43 | 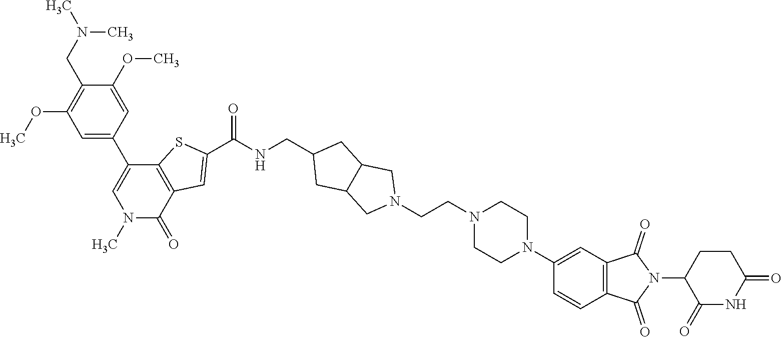 [SMILES: COc1cc(-c2cn(C)c(=O)c3cc(C(=O)NCC4CC5CN(CCN6CCN(c7ccc8c(c7)C(=O)N(C7CCC(=O)NC7=O)C8=O)CC6)CC5C4)sc23)cc(OC)c1CN(C)C] |
| G44 | 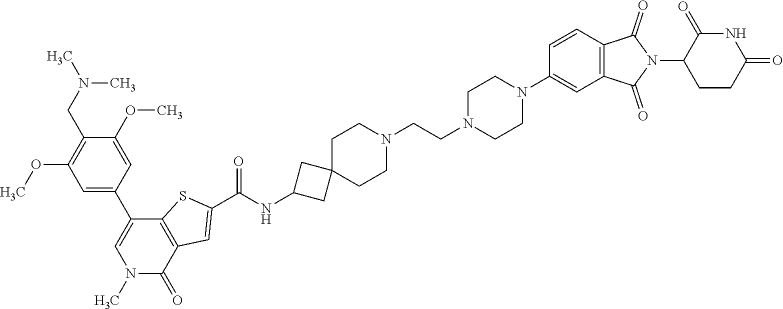 [SMILES: COc1cc(-c2cn(C)c(=O)c3cc(C(=O)NC4CC5(CCN(CCN6CCN(c7ccc8c(c7)C(=O)N(C7CCC(=O)NC7=O)C8=O)CC6)CC5)C4)sc23)cc(OC)c1CN(C)C] |
| G45 | 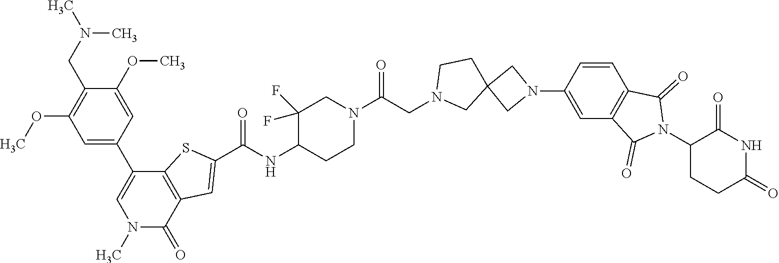 [SMILES: COc1cc(-c2cn(C)c(=O)c3cc(C(=O)NC4CCN(C(=O)CN5CCC6(C5)CN(c5ccc7c(c5)C(=O)N(C5CCC(=O)NC5=O)C7=O)C6)CC4(F)F)sc23)cc(OC)c1CN(C)C] |
| G46 | 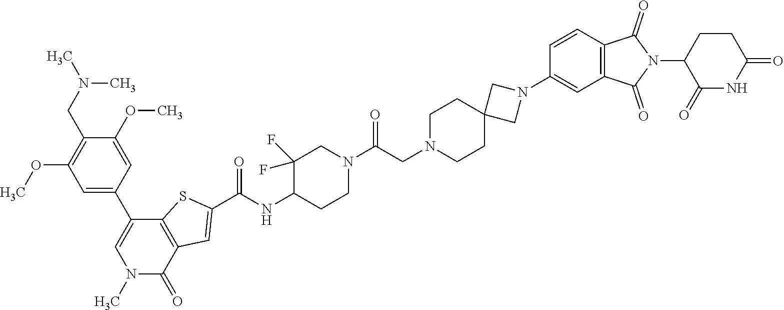 [SMILES: COc1cc(-c2cn(C)c(=O)c3cc(C(=O)NC4CCN(C(=O)CN5CCC6(CC5)CN(c5ccc7c(c5)C(=O)N(C5CCC(=O)NC5=O)C7=O)C6)CC4(F)F)sc23)cc(OC)c1CN(C)C] |
| G47 | 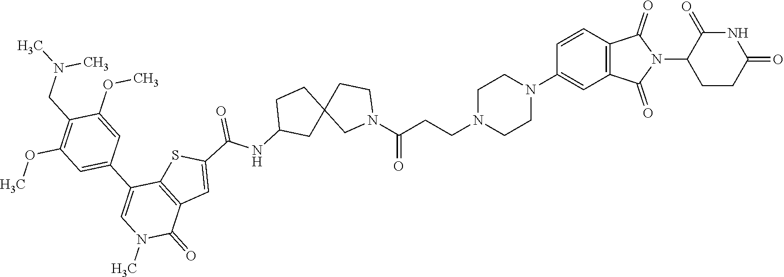 [SMILES: COc1cc(-c2cn(C)c(=O)c3cc(C(=O)NC4CCC5(CCN(C(=O)CCN6CCN(c7ccc8c(c7)C(=O)N(C7CCC(=O)NC7=O)C8=O)CC6)C5)C4)sc23)cc(OC)c1CN(C)C] |
| G48 | 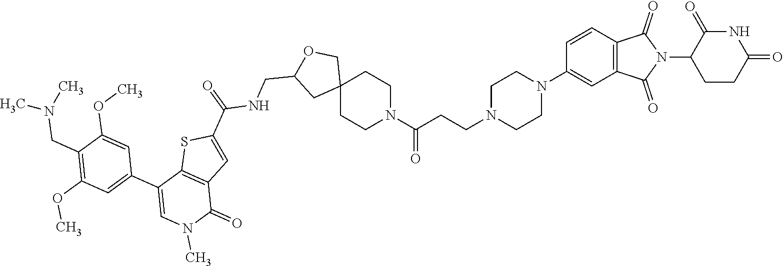 [SMILES: COc1cc(-c2cn(C)c(=O)c3cc(C(=O)NCC4CC5(CCN(C(=O)CCN6CCN(c7ccc8c(c7)C(=O)N(C7CCC(=O)NC7=O)C8=O)CC6)CC5)CO4)sc23)cc(OC)c1CN(C)C] |
| G49 | 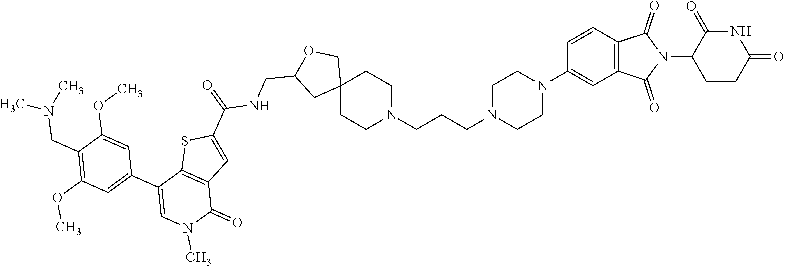 [SMILES: COc1cc(-c2cn(C)c(=O)c3cc(C(=O)NCC4CC5(CCN(CCCN6CCN(c7ccc8c(c7)C(=O)N(C7CCC(=O)NC7=O)C8=O)CC6)CC5)CO4)sc23)cc(OC)c1CN(C)C] |
| G50 | 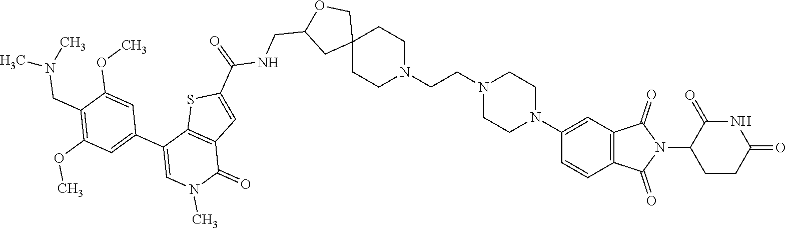 [SMILES: COc1cc(-c2cn(C)c(=O)c3cc(C(=O)NCC4CC5(CCN(CCN6CCN(c7ccc8c(c7)C(=O)N(C7CCC(=O)NC7=O)C8=O)CC6)CC5)CO4)sc23)cc(OC)c1CN(C)C] |
| G51 | 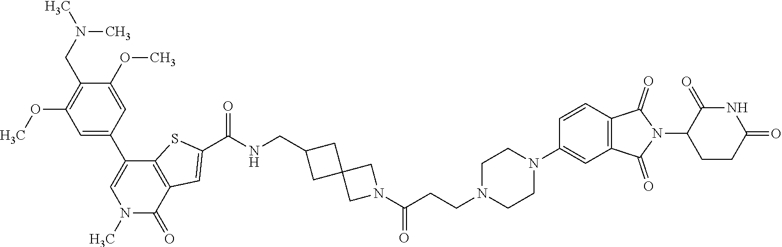 [SMILES: COc1cc(-c2cn(C)c(=O)c3cc(C(=O)NCC4CC5(C4)CN(C(=O)CCN4CCN(c6ccc7c(c6)C(=O)N(C6CCC(=O)NC6=O)C7=O)CC4)C5)sc23)cc(OC)c1CN(C)C] |
| G52 | 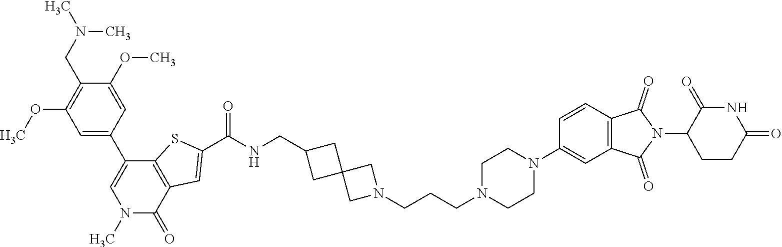 [SMILES: COc1cc(-c2cn(C)c(=O)c3cc(C(=O)NCC4CC5(C4)CN(CCCN4CCN(c6ccc7c(c6)C(=O)N(C6CCC(=O)NC6=O)C7=O)CC4)C5)sc23)cc(OC)c1CN(C)C] |
| G53 | 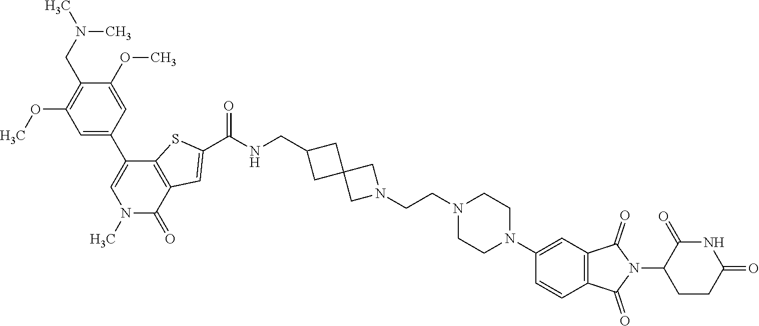 [SMILES: COc1cc(-c2cn(C)c(=O)c3cc(C(=O)NCC4CC5(C4)CN(CCN4CCN(c6ccc7c(c6)C(=O)N(C6CCC(=O)NC6=O)C7=O)CC4)C5)sc23)cc(OC)c1CN(C)C] |
| G54 | 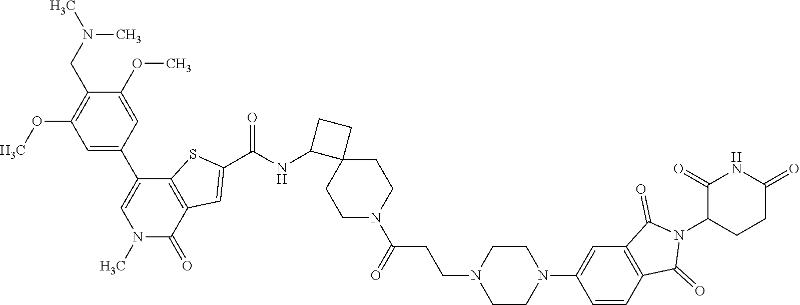 [SMILES: COc1cc(-c2cn(C)c(=O)c3cc(C(=O)NC4CCC45CCN(C(=O)CCN4CCN(c6ccc7c(c6)C(=O)N(C6CCC(=O)NC6=O)C7=O)CC4)CC5)sc23)cc(OC)c1CN(C)C] |
| G55 | 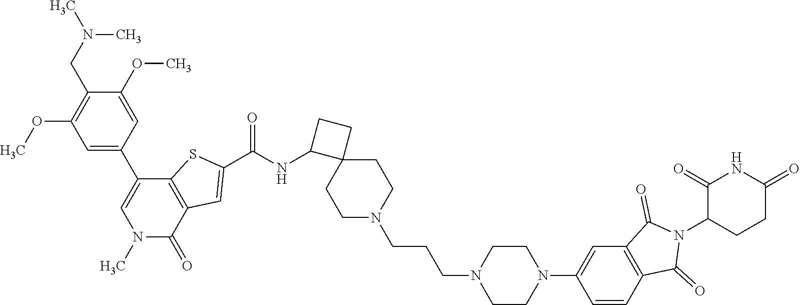 [SMILES: COc1cc(-c2cn(C)c(=O)c3cc(C(=O)NC4CCC45CCN(CCCN4CCN(c6ccc7c(c6)C(=O)N(C6CCC(=O)NC6=O)C7=O)CC4)CC5)sc23)cc(OC)c1CN(C)C] |
| G56 | 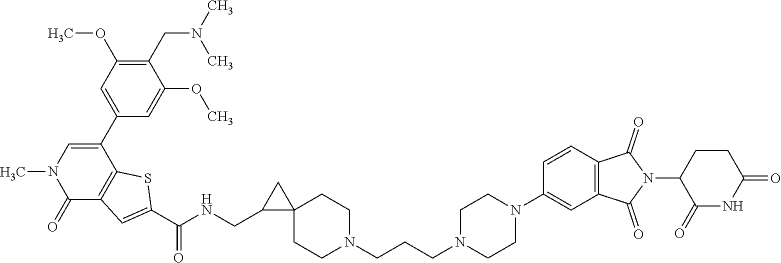 [SMILES: COc1cc(-c2cn(C)c(=O)c3cc(C(=O)NCC4CC45CCN(CCCN4CCN(c6ccc7c(c6)C(=O)N(C6CCC(=O)NC6=O)C7=O)CC4)CC5)sc23)cc(OC)c1CN(C)C] |
| G57 | 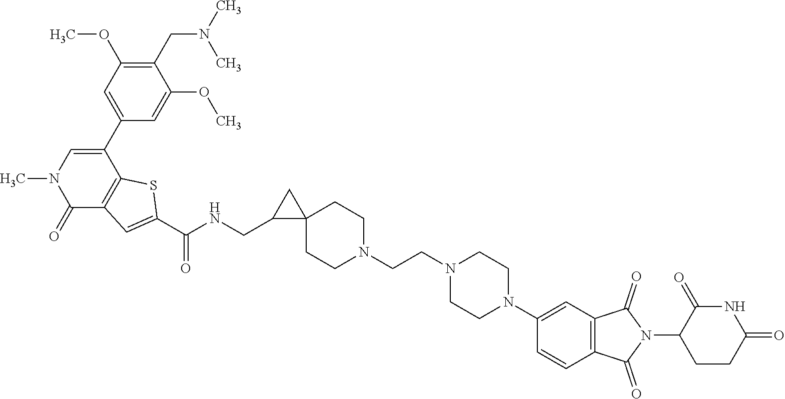 [SMILES: COc1cc(-c2cn(C)c(=O)c3cc(C(=O)NCC4CC45CCN(CCN4CCN(c6ccc7c(c6)C(=O)N(C6CCC(=O)NC6=O)C7=O)CC4)CC5)sc23)cc(OC)c1CN(C)C] |
| G58 | 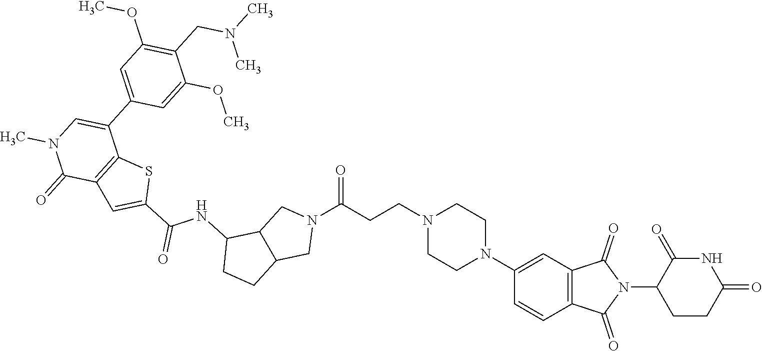 [SMILES: COc1cc(-c2cn(C)c(=O)c3cc(C(=O)NC4CCC5CN(C(=O)CCN6CCN(c7ccc8c(c7)C(=O)N(C7CCC(=O)NC7=O)C8=O)CC6)CC54)sc23)cc(OC)c1CN(C)C] |
| G59 | 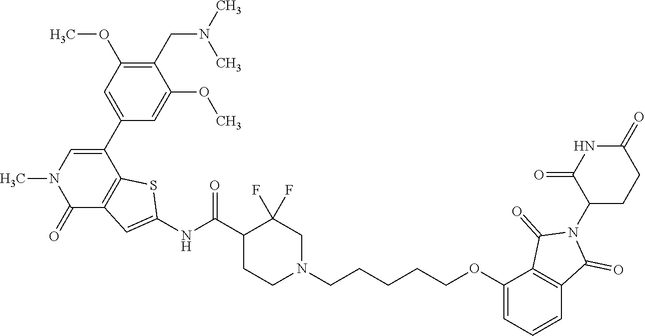 [SMILES: COc1cc(-c2cn(C)c(=O)c3cc(NC(=O)C4CCN(CCCCCOc5cccc6c5C(=O)N(C5CCC(=O)NC5=O)C6=O)CC4(F)F)sc23)cc(OC)c1CN(C)C] |
| G60 | 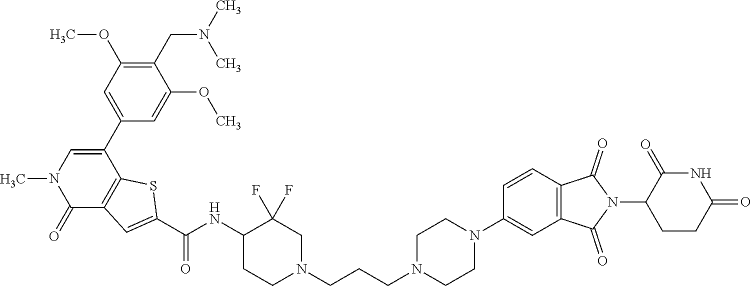 [SMILES: COc1cc(-c2cn(C)c(=O)c3cc(C(=O)NC4CCN(CCCN5CCN(c6ccc7c(c6)C(=O)N(C6CCC(=O)NC6=O)C7=O)CC5)CC4(F)F)sc23)cc(OC)c1CN(C)C] |
| G61 | 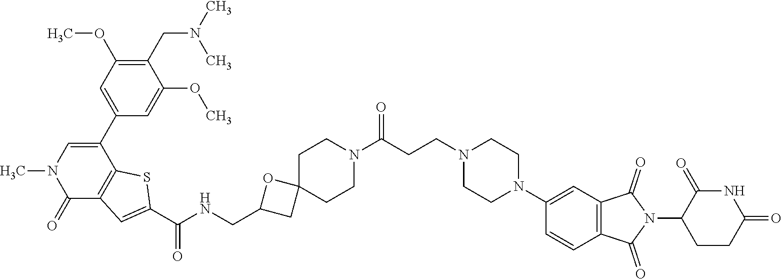 [SMILES: COc1cc(-c2cn(C)c(=O)c3cc(C(=O)NCC4CC5(CCN(C(=O)CCN6CCN(c7ccc8c(c7)C(=O)N(C7CCC(=O)NC7=O)C8=O)CC6)CC5)O4)sc23)cc(OC)c1CN(C)C] |
| G62 | 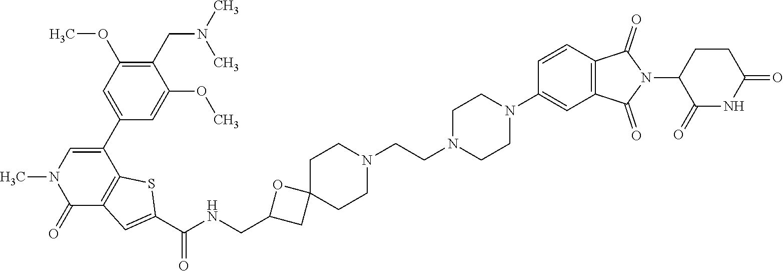 [SMILES: COc1cc(-c2cn(C)c(=O)c3cc(C(=O)NCC4CC5(CCN(CCN6CCN(c7ccc8c(c7)C(=O)N(C7CCC(=O)NC7=O)C8=O)CC6)CC5)O4)sc23)cc(OC)c1CN(C)C] |
| G63 | 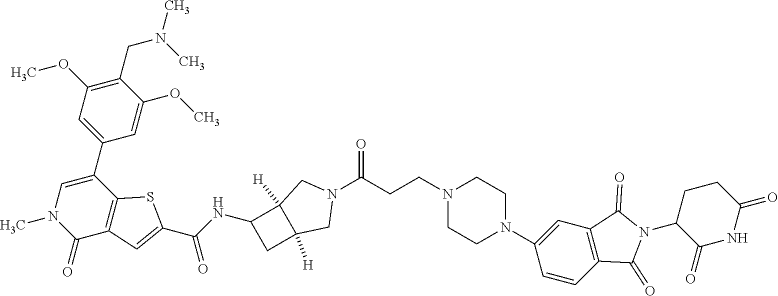 [SMILES: COc1cc(-c2cn(C)c(=O)c3cc(C(=O)NC4C[C@@H]5CN(C(=O)CCN6CCN(c7ccc8c(c7)C(=O)N(C7CCC(=O)NC7=O)C8=O)CC6)C[C@H]45)sc23)cc(OC)c1CN(C)C] |
| G64 | 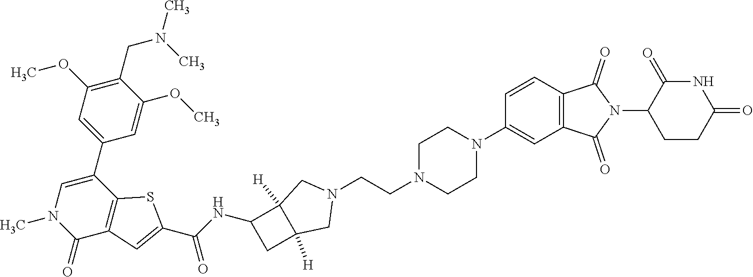 [SMILES: COc1cc(-c2cn(C)c(=O)c3cc(C(=O)NC4C[C@@H]5CN(CCN6CCN(c7ccc8c(c7)C(=O)N(C7CCC(=O)NC7=O)C8=O)CC6)C[C@H]45)sc23)cc(OC)c1CN(C)C] |
| G65 | 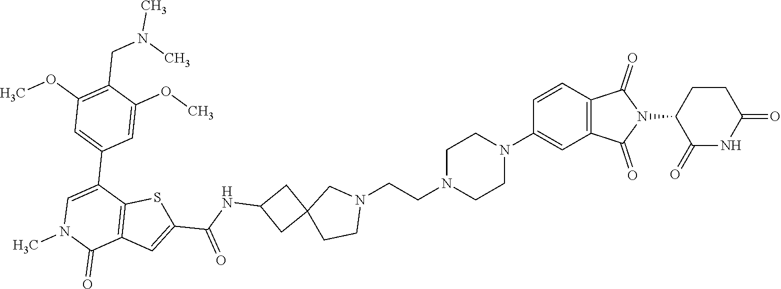 [SMILES: COc1cc(-c2cn(C)c(=O)c3cc(C(=O)NC4CC5(CCN(CCN6CCN(c7ccc8c(c7)C(=O)N([C@@H]7CCC(=O)NC7=O)C8=O)CC6)C5)C4)sc23)cc(OC)c1CN(C)C] |
| G66 | 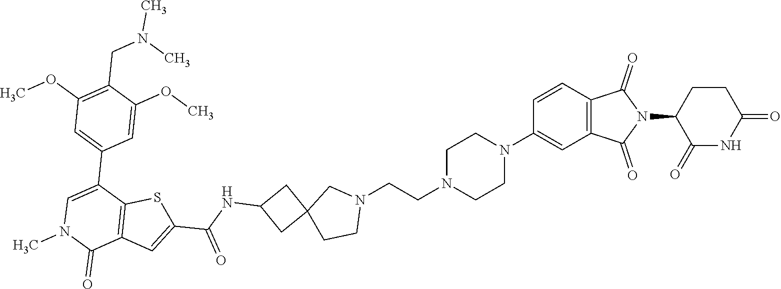 [SMILES: COc1cc(-c2cn(C)c(=O)c3cc(C(=O)NC4CC5(CCN(CCN6CCN(c7ccc8c(c7)C(=O)N([C@H]7CCC(=O)NC7=O)C8=O)CC6)C5)C4)sc23)cc(OC)c1CN(C)C] |
| G67 | 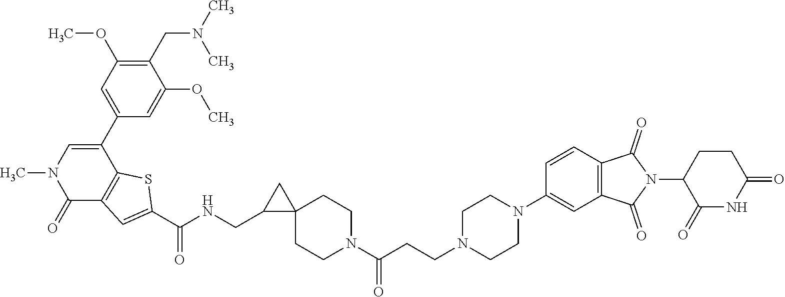 [SMILES: COc1cc(-c2cn(C)c(=O)c3cc(C(=O)NCC4CC45CCN(C(=O)CCN4CCN(c6ccc7c(c6)C(=O)N(C6CCC(=O)NC6=O)C7=O)CC4)CC5)sc23)cc(OC)c1CN(C)C] |
| G68 | 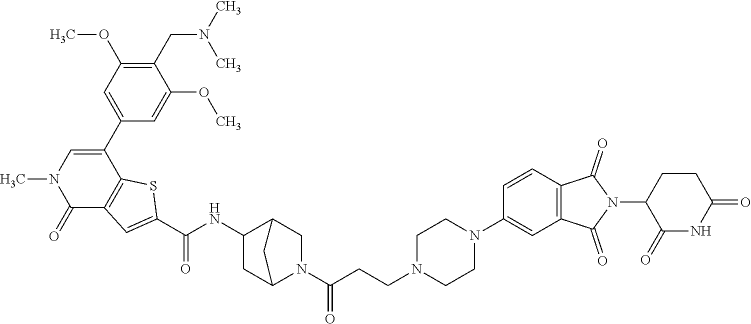 [SMILES: COc1cc(-c2cn(C)c(=O)c3cc(C(=O)NC4CC5CC4CN5C(=O)CCN4CCN(c5ccc6c(c5)C(=O)N(C5CCC(=O)NC5=O)C6=O)CC4)sc23)cc(OC)c1CN(C)C] |
| G69 | 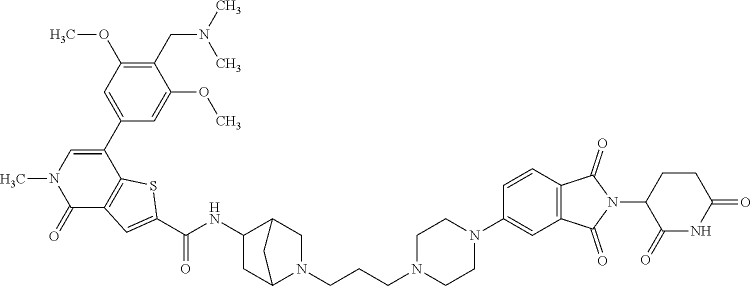 [SMILES: COc1cc(-c2cn(C)c(=O)c3cc(C(=O)NC4CC5CC4CN5CCCN4CCN(c5ccc6c(c5)C(=O)N(C5CCC(=O)NC5=O)C6=O)CC4)sc23)cc(OC)c1CN(C)C] |
| G70 | 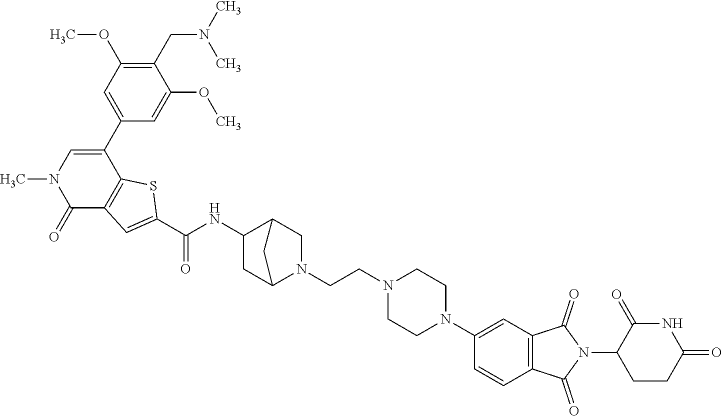 [SMILES: COc1cc(-c2cn(C)c(=O)c3cc(C(=O)NC4CC5CC4CN5CCN4CCN(c5ccc6c(c5)C(=O)N(C5CCC(=O)NC5=O)C6=O)CC4)sc23)cc(OC)c1CN(C)C] |
| G71 | 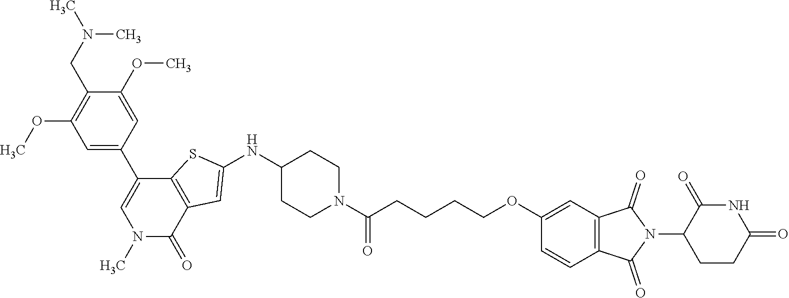 [SMILES: COc1cc(-c2cn(C)c(=O)c3cc(NC4CCN(C(=O)CCCCOc5ccc6c(c5)C(=O)N(C5CCC(=O)NC5=O)C6=O)CC4)sc23)cc(OC)c1CN(C)C] |
| G72 | 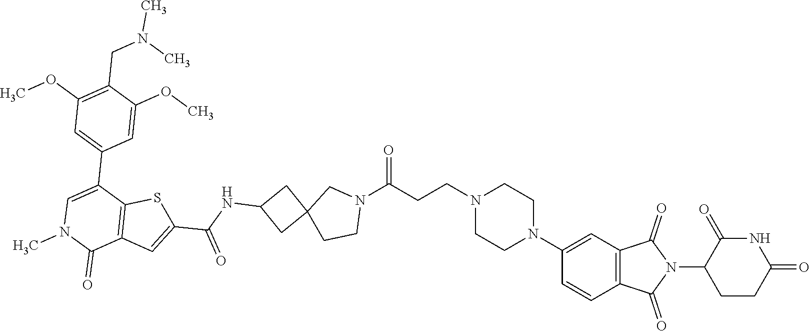 [SMILES: COc1cc(-c2cn(C)c(=O)c3cc(C(=O)NC4CC5(CCN(C(=O)CCN6CCN(c7ccc8c(c7)C(=O)N(C7CCC(=O)NC7=O)C8=O)CC6)C5)C4)sc23)cc(OC)c1CN(C)C] |
| G73 | 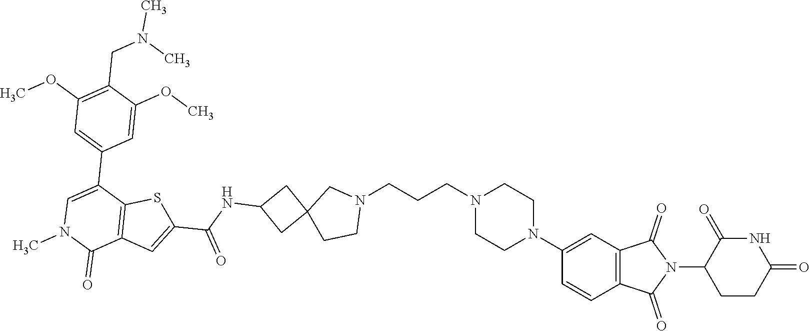 [SMILES: COc1cc(-c2cn(C)c(=O)c3cc(C(=O)NC4CC5(CCN(CCCN6CCN(c7ccc8c(c7)C(=O)N(C7CCC(=O)NC7=O)C8=O)CC6)C5)C4)sc23)cc(OC)c1CN(C)C] |
| G74 | 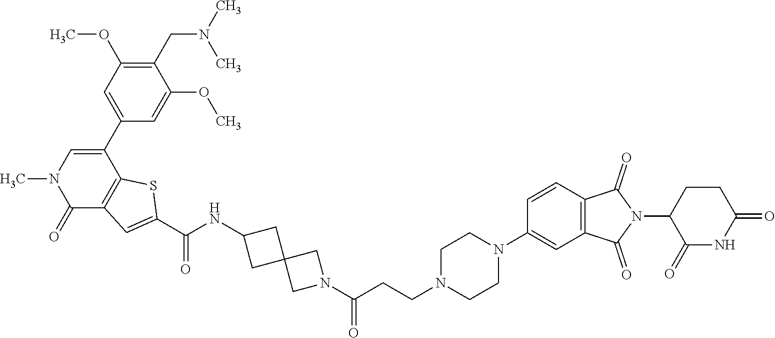 [SMILES: COc1cc(-c2cn(C)c(=O)c3cc(C(=O)NC4CC5(C4)CN(C(=O)CCN4CCN(c6ccc7c(c6)C(=O)N(C6CCC(=O)NC6=O)C7=O)CC4)C5)sc23)cc(OC)c1CN(C)C] |
| G75 | 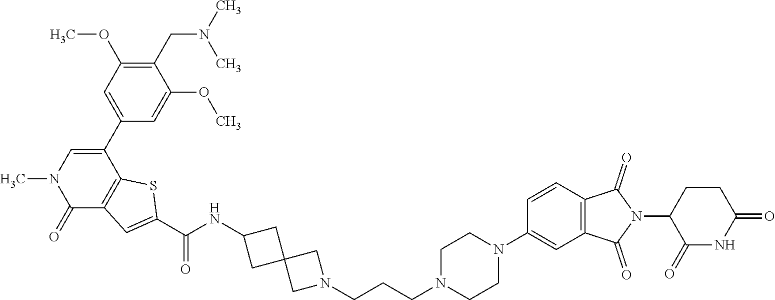 [SMILES: COc1cc(-c2cn(C)c(=O)c3cc(C(=O)NC4CC5(C4)CN(CCCN4CCN(c6ccc7c(c6)C(=O)N(C6CCC(=O)NC6=O)C7=O)CC4)C5)sc23)cc(OC)c1CN(C)C] |
| G76 | 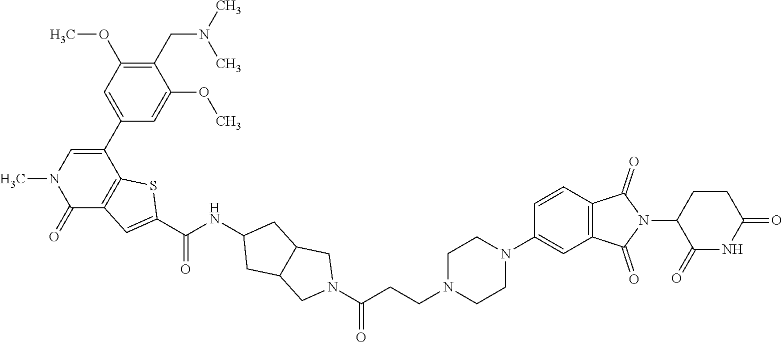 [SMILES: COc1cc(-c2cn(C)c(=O)c3cc(C(=O)NC4CC5CN(C(=O)CCN6CCN(c7ccc8c(c7)C(=O)N(C7CCC(=O)NC7=O)C8=O)CC6)CC5C4)sc23)cc(OC)c1CN(C)C] |
| G77 | 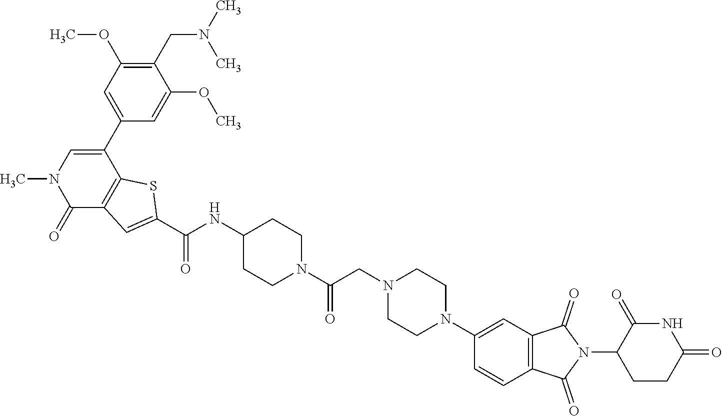 [SMILES: COc1cc(-c2cn(C)c(=O)c3cc(C(=O)NC4CCN(C(=O)CN5CCN(c6ccc7c(c6)C(=O)N(C6CCC(=O)NC6=O)C7=O)CC5)CC4)sc23)cc(OC)c1CN(C)C] |
| G78 | 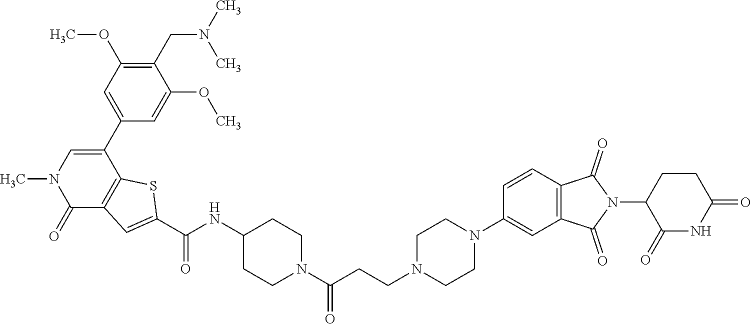 [SMILES: COc1cc(-c2cn(C)c(=O)c3cc(C(=O)NC4CCN(C(=O)CCN5CCN(c6ccc7c(c6)C(=O)N(C6CCC(=O)NC6=O)C7=O)CC5)CC4)sc23)cc(OC)c1CN(C)C] |
| G79 | 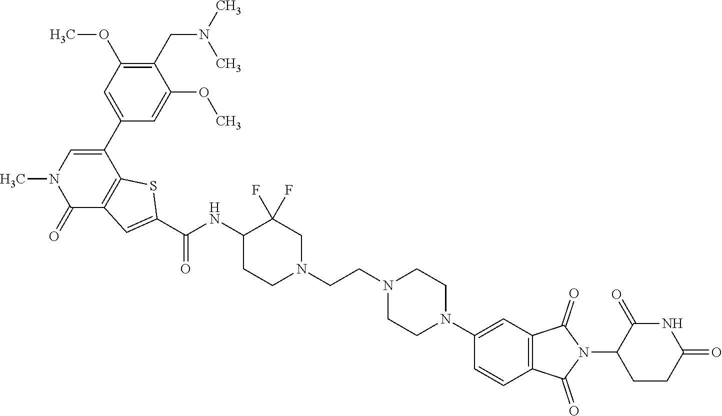 [SMILES: COc1cc(-c2cn(C)c(=O)c3cc(C(=O)NC4CCN(CCN5CCN(c6ccc7c(c6)C(=O)N(C6CCC(=O)NC6=O)C7=O)CC5)CC4(F)F)sc23)cc(OC)c1CN(C)C] |
| G80 | 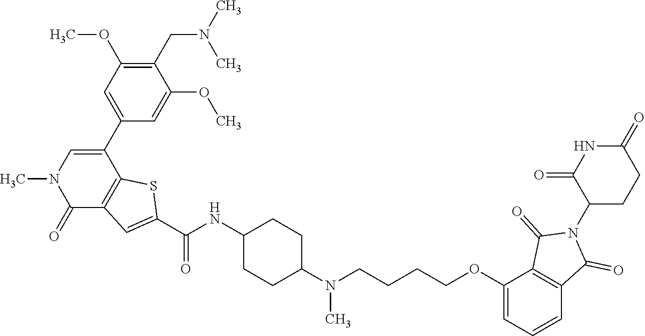 [SMILES: COc1cc(-c2cn(C)c(=O)c3cc(C(=O)NC4CCC(N(C)CCCCOc5cccc6c5C(=O)N(C5CCC(=O)NC5=O)C6=O)CC4)sc23)cc(OC)c1CN(C)C] |
| G81 | 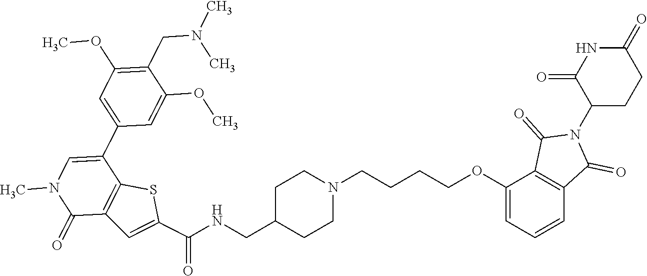 [SMILES: COc1cc(-c2cn(C)c(=O)c3cc(C(=O)NCC4CCN(CCCCOc5cccc6c5C(=O)N(C5CCC(=O)NC5=O)C6=O)CC4)sc23)cc(OC)c1CN(C)C] |
| G82 | 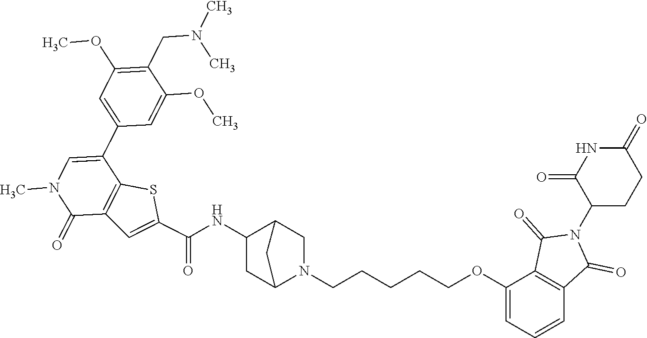 [SMILES: COc1cc(-c2cn(C)c(=O)c3cc(C(=O)NC4CC5CC4CN5CCCCCOc4cccc5c4C(=O)N(C4CCC(=O)NC4=O)C5=O)sc23)cc(OC)c1CN(C)C] |
| G83 | 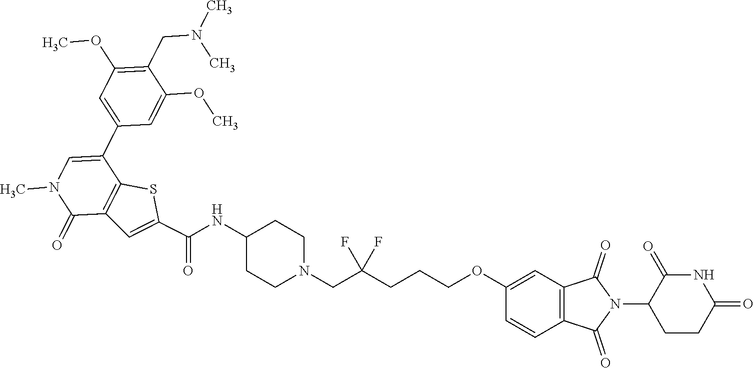 [SMILES: COc1cc(-c2cn(C)c(=O)c3cc(C(=O)NC4CCN(CC(F)(F)CCCOc5ccc6c(c5)C(=O)N(C5CCC(=O)NC5=O)C6=O)CC4)sc23)cc(OC)c1CN(C)C] |
| G84 | 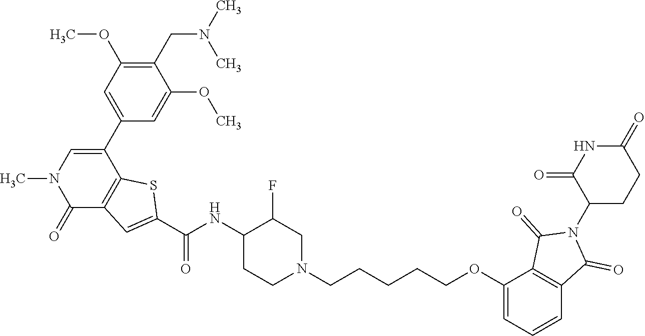 [SMILES: COc1cc(-c2cn(C)c(=O)c3cc(C(=O)NC4CCN(CCCCCOc5cccc6c5C(=O)N(C5CCC(=O)NC5=O)C6=O)CC4F)sc23)cc(OC)c1CN(C)C] |
| G85 | 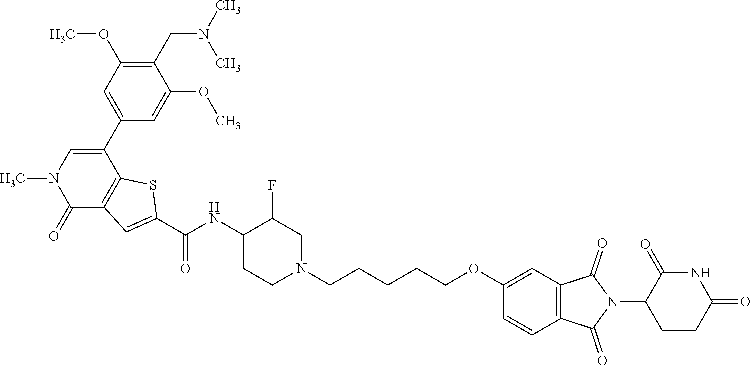 [SMILES: COc1cc(-c2cn(C)c(=O)c3cc(C(=O)NC4CCN(CCCCCOc5ccc6c(c5)C(=O)N(C5CCC(=O)NC5=O)C6=O)CC4F)sc23)cc(OC)c1CN(C)C] |
| G86 | 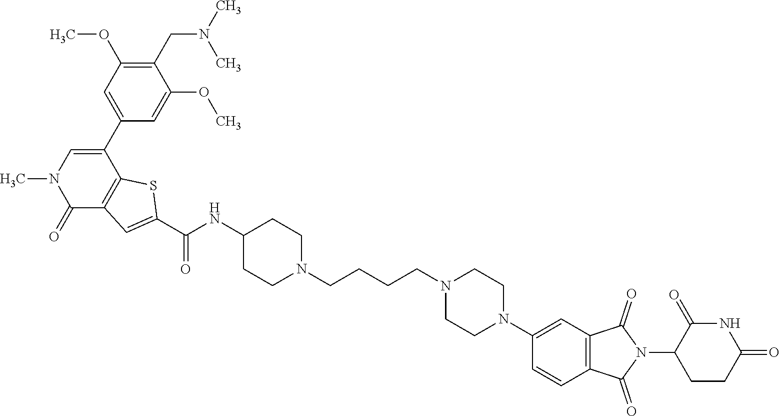 [SMILES: COc1cc(-c2cn(C)c(=O)c3cc(C(=O)NC4CCN(CCCCN5CCN(c6ccc7c(c6)C(=O)N(C6CCC(=O)NC6=O)C7=O)CC5)CC4)sc23)cc(OC)c1CN(C)C] |
| G87 | 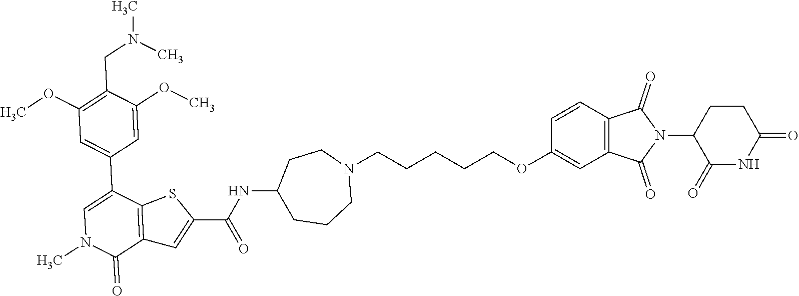 [SMILES: COc1cc(-c2cn(C)c(=O)c3cc(C(=O)NC4CCCN(CCCCCOc5ccc6c(c5)C(=O)N(C5CCC(=O)NC5=O)C6=O)CC4)sc23)cc(OC)c1CN(C)C] |
| G88 | 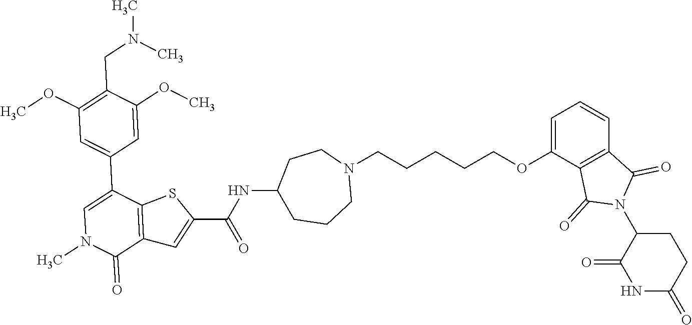 [SMILES: COc1cc(-c2cn(C)c(=O)c3cc(C(=O)NC4CCCN(CCCCCOc5cccc6c5C(=O)N(C5CCC(=O)NC5=O)C6=O)CC4)sc23)cc(OC)c1CN(C)C] |
| G89 | 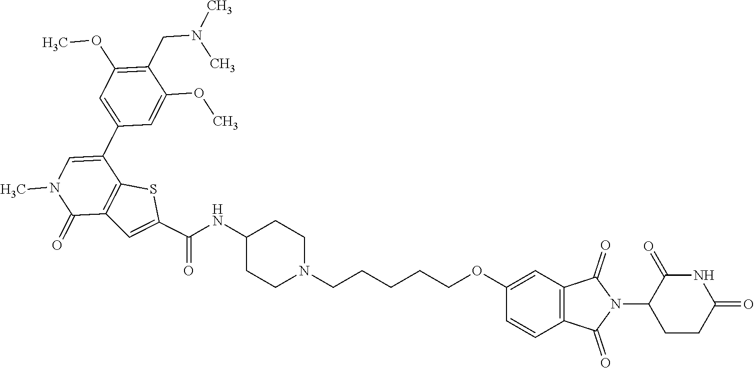 [SMILES: COc1cc(-c2cn(C)c(=O)c3cc(C(=O)NC4CCN(CCCCCOc5ccc6c(c5)C(=O)N(C5CCC(=O)NC5=O)C6=O)CC4)sc23)cc(OC)c1CN(C)C] |
| G90 | 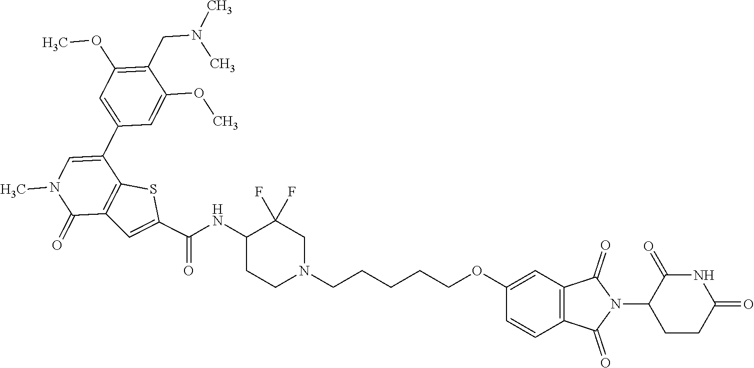 [SMILES: COc1cc(-c2cn(C)c(=O)c3cc(C(=O)NC4CCN(CCCCCOc5ccc6c(c5)C(=O)N(C5CCC(=O)NC5=O)C6=O)CC4(F)F)sc23)cc(OC)c1CN(C)C] |
| G91 | 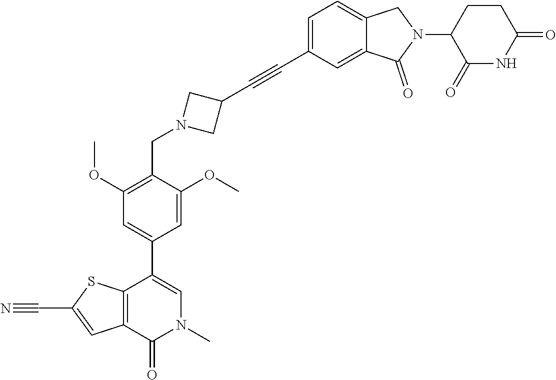 [SMILES: COc1cc(-c2cn(C)c(=O)c3cc(C#N)sc23)cc(OC)c1CN1CC(C#Cc2ccc3c(c2)C(=O)N(C2CCC(=O)NC2=O)C3)C1] |
| G92 | 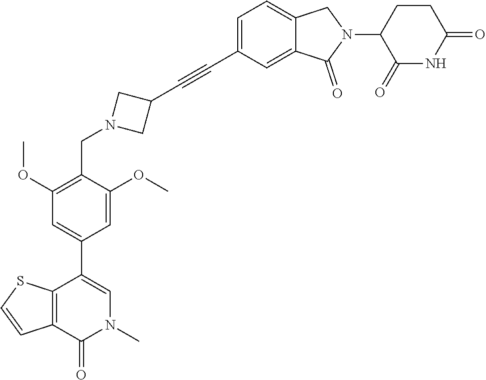 [SMILES: COc1cc(-c2cn(C)c(=O)c3ccsc23)cc(OC)c1CN1CC(C#Cc2ccc3c(c2)C(=O)N(C2CCC(=O)NC2=O)C3)C1] |
| G93 | 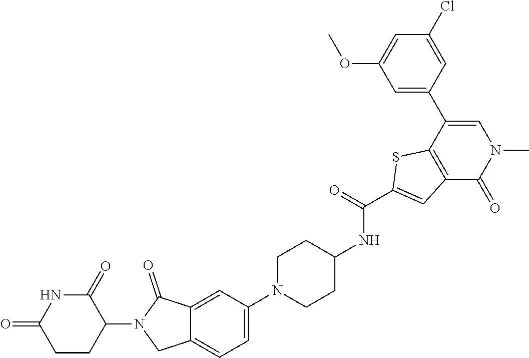 [SMILES: COc1cc(Cl)cc(-c2cn(C)c(=O)c3cc(C(=O)NC4CCN(c5ccc6c(c5)C(=O)N(C5CCC(=O)NC5=O)C6)CC4)sc23)c1] |
| G94 | 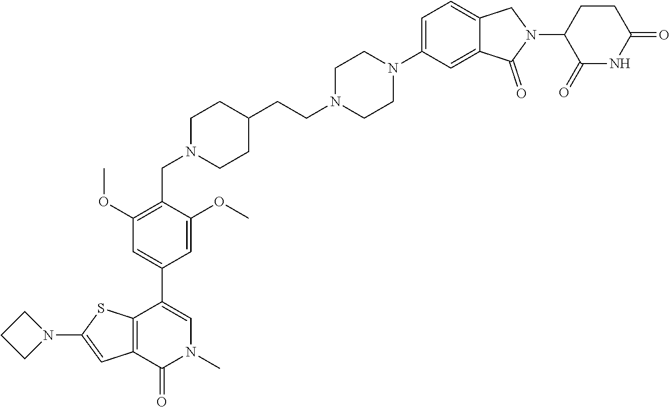 [SMILES: COc1cc(-c2cn(C)c(=O)c3cc(N4CCC4)sc23)cc(OC)c1CN1CCC(CCN2CCN(c3ccc4c(c3)C(=O)N(C3CCC(=O)NC3=O)C4)CC2)CC1] |
| G95 | 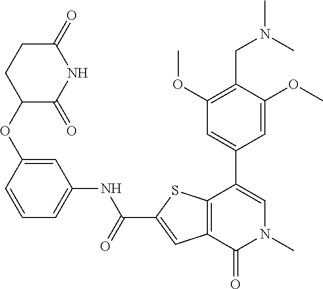 [SMILES: COc1cc(-c2cn(C)c(=O)c3cc(C(=O)Nc4cccc(OC5CCC(=O)NC5=O)c4)sc23)cc(OC)c1CN(C)C] |
| G96 | 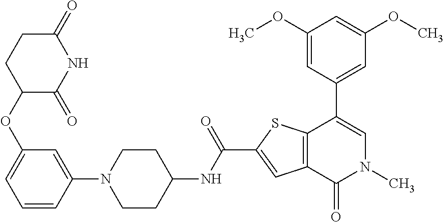 [SMILES: COc1cc(OC)cc(-c2cn(C)c(=O)c3cc(C(=O)NC4CCN(c5cccc(OC6CCC(=O)NC6=O)c5)CC4)sc23)c1] |
| G97 | 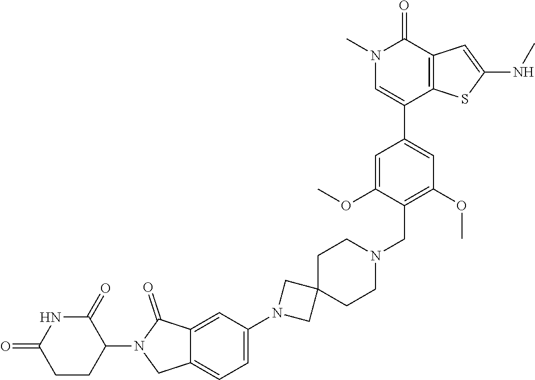 [SMILES: CNc1cc2c(=O)n(C)cc(-c3cc(OC)c(CN4CCC5(CC4)CN(c4ccc6c(c4)C(=O)N(C4CCC(=O)NC4=O)C6)C5)c(OC)c3)c2s1] |
| G98 | 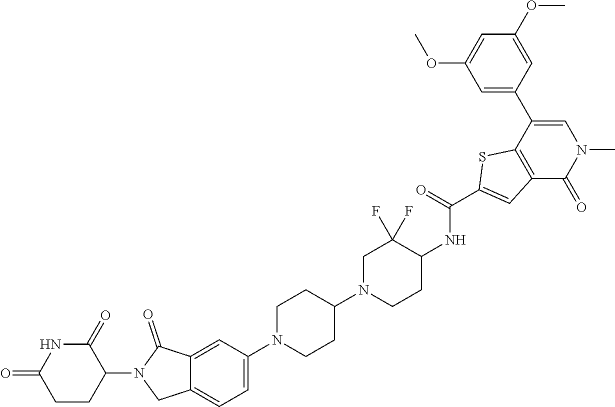 [SMILES: COc1cc(OC)cc(-c2cn(C)c(=O)c3cc(C(=O)NC4CCN(C5CCN(c6ccc7c(c6)C(=O)N(C6CCC(=O)NC6=O)C7)CC5)CC4(F)F)sc23)c1] |
| G99 | 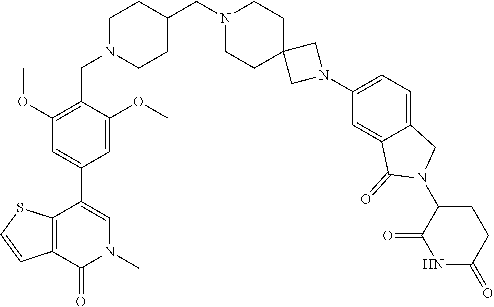 [SMILES: COc1cc(-c2cn(C)c(=O)c3ccsc23)cc(OC)c1CN1CCC(CN2CCC3(CC2)CN(c2ccc4c(c2)C(=O)N(C2CCC(=O)NC2=O)C4)C3)CC1] |
| G100 | 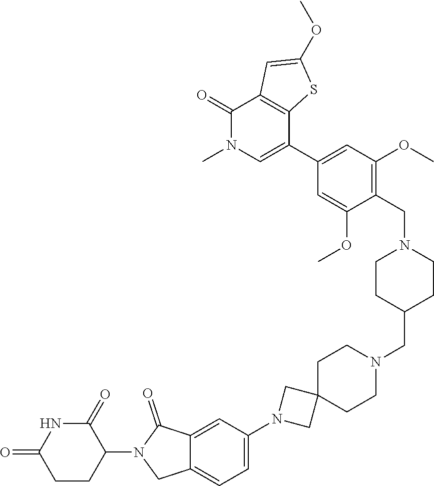 [SMILES: COc1cc2c(=O)n(C)cc(-c3cc(OC)c(CN4CCC(CN5CCC6(CC5)CN(c5ccc7c(c5)C(=O)N(C5CCC(=O)NC5=O)C7)C6)CC4)c(OC)c3)c2s1] |
| G101 | 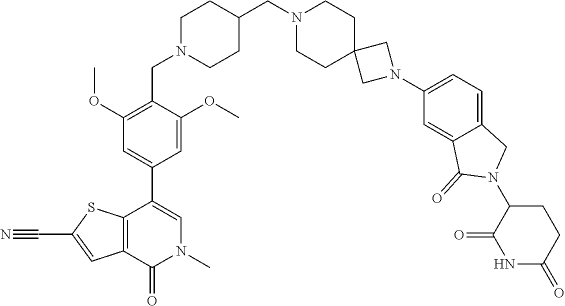 [SMILES: COc1cc(-c2cn(C)c(=O)c3cc(C#N)sc23)cc(OC)c1CN1CCC(CN2CCC3(CC2)CN(c2ccc4c(c2)C(=O)N(C2CCC(=O)NC2=O)C4)C3)CC1] |
| G102 | 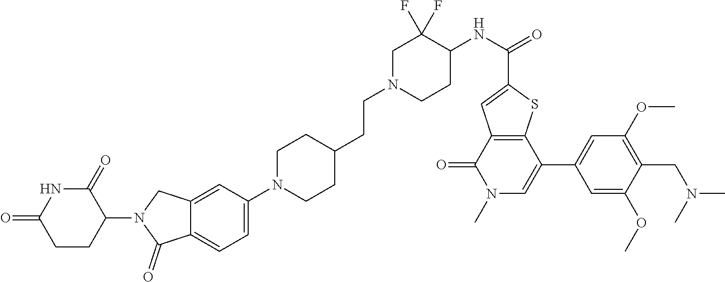 [SMILES: COc1cc(-c2cn(C)c(=O)c3cc(C(=O)NC4CCN(CCC5CCN(c6ccc7c(c6)CN(C6CCC(=O)NC6=O)C7=O)CC5)CC4(F)F)sc23)cc(OC)c1CN(C)C] |
| G103 | 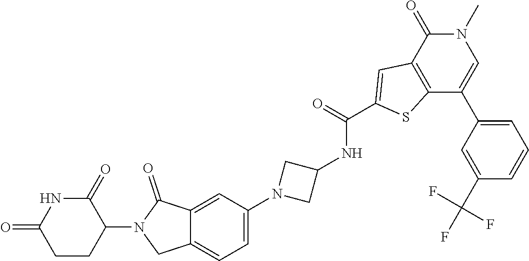 [SMILES: Cn1cc(-c2cccc(C(F)(F)F)c2)c2sc(C(=O)NC3CN(c4ccc5c(c4)C(=O)N(C4CCC(=O)NC4=O)C5)C3)cc2c1=O] |
| G104 | 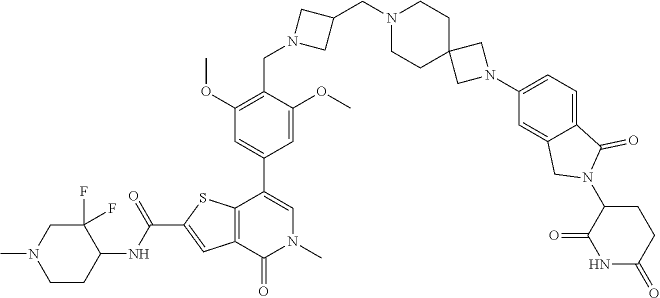 [SMILES: COc1cc(-c2cn(C)c(=O)c3cc(C(=O)NC4CCN(C)CC4(F)F)sc23)cc(OC)c1CN1CC(CN2CCC3(CC2)CN(c2ccc4c(c2)CN(C2CCC(=O)NC2=O)C4=O)C3)C1] |
| G105 | 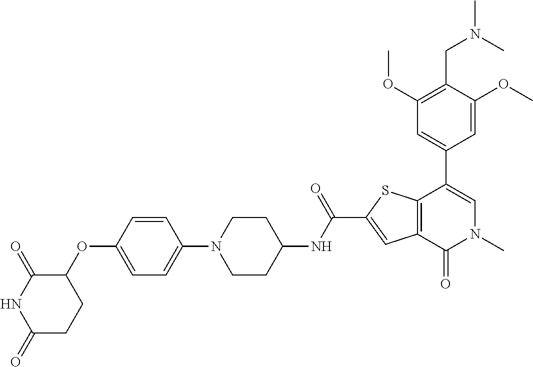 [SMILES: COc1cc(-c2cn(C)c(=O)c3cc(C(=O)NC4CCN(c5ccc(OC6CCC(=O)NC6=O)cc5)CC4)sc23)cc(OC)c1CN(C)C] |
| G106 | 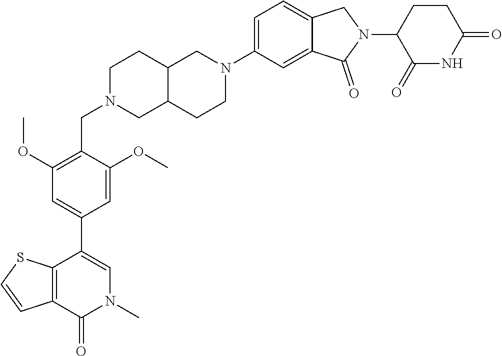 [SMILES: COc1cc(-c2cn(C)c(=O)c3ccsc23)cc(OC)c1CN1CCC2CN(c3ccc4c(c3)C(=O)N(C3CCC(=O)NC3=O)C4)CCC2C1] |
| G107 | 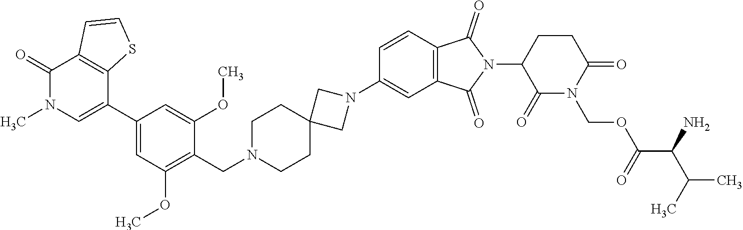 [SMILES: COc1cc(-c2cn(C)c(=O)c3ccsc23)cc(OC)c1CN1CCC2(CC1)CN(c1ccc3c(c1)C(=O)N(C1CCC(=O)N(COC(=O)[C@@H](N)C(C)C)C1=O)C3=O)C2] |
| G108 | 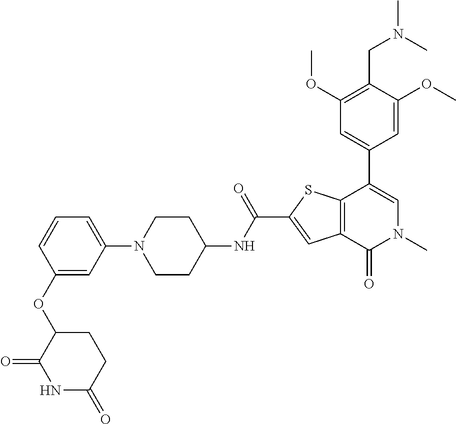 [SMILES: COc1cc(-c2cn(C)c(=O)c3cc(C(=O)NC4CCN(c5cccc(OC6CCC(=O)NC6=O)c5)CC4)sc23)cc(OC)c1CN(C)C] |
| G109 | 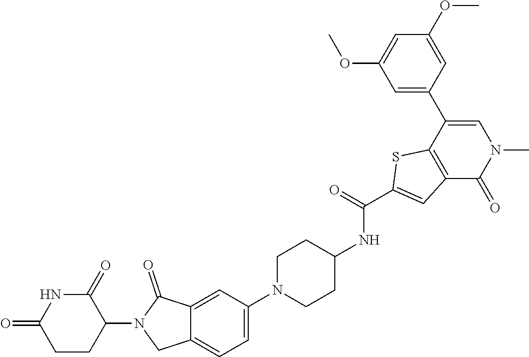 [SMILES: COc1cc(OC)cc(-c2cn(C)c(=O)c3cc(C(=O)NC4CCN(c5ccc6c(c5)C(=O)N(C5CCC(=O)NC5=O)C6)CC4)sc23)c1] |
| G110 | 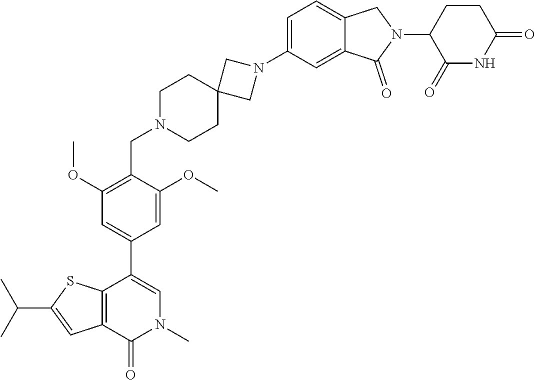 [SMILES: COc1cc(-c2cn(C)c(=O)c3cc(C(C)C)sc23)cc(OC)c1CN1CCC2(CC1)CN(c1ccc3c(c1)C(=O)N(C1CCC(=O)NC1=O)C3)C2] |
| G111 | 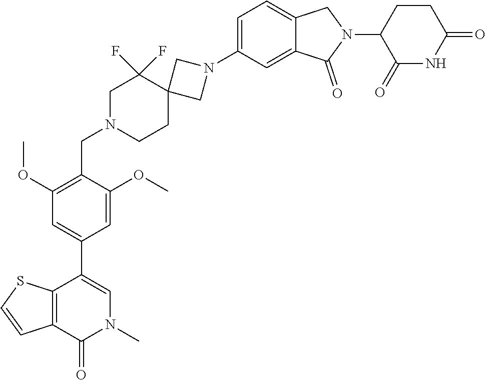 [SMILES: COc1cc(-c2cn(C)c(=O)c3ccsc23)cc(OC)c1CN1CCC2(CN(c3ccc4c(c3)C(=O)N(C3CCC(=O)NC3=O)C4)C2)C(F)(F)C1] |
| G112 | 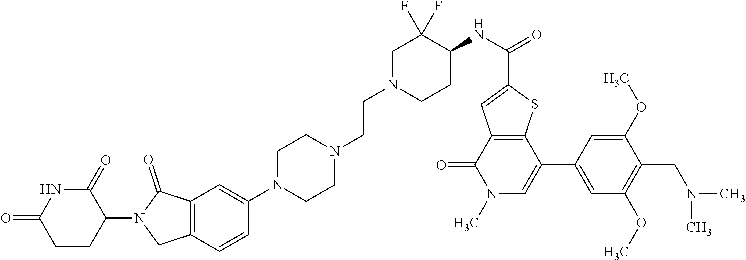 [SMILES: COc1cc(-c2cn(C)c(=O)c3cc(C(=O)N[C@H]4CCN(CCN5CCN(c6ccc7c(c6)C(=O)N(C6CCC(=O)NC6=O)C7)CC5)CC4(F)F)sc23)cc(OC)c1CN(C)C] |
| G113 | 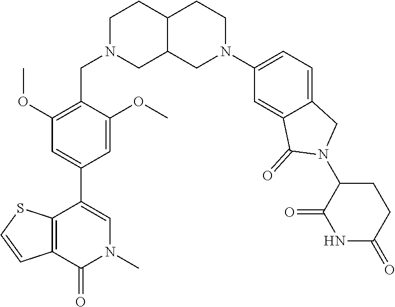 [SMILES: COc1cc(-c2cn(C)c(=O)c3ccsc23)cc(OC)c1CN1CCC2CCN(c3ccc4c(c3)C(=O)N(C3CCC(=O)NC3=O)C4)CC2C1] |
| G114 | 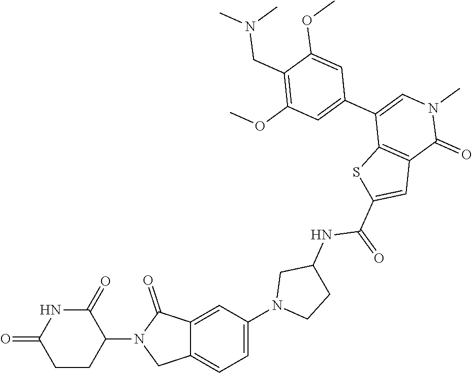 [SMILES: COc1cc(-c2cn(C)c(=O)c3cc(C(=O)NC4CCN(c5ccc6c(c5)C(=O)N(C5CCC(=O)NC5=O)C6)C4)sc23)cc(OC)c1CN(C)C] |
| G115 | 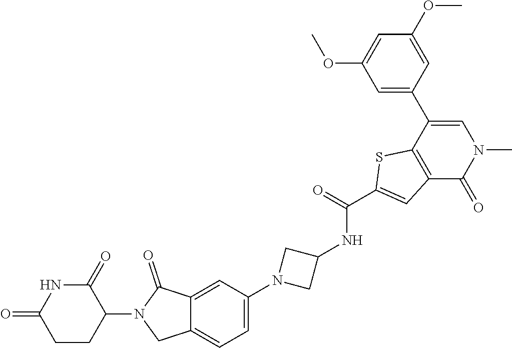 [SMILES: COc1cc(OC)cc(-c2cn(C)c(=O)c3cc(C(=O)NC4CN(c5ccc6c(c5)C(=O)N(C5CCC(=O)NC5=O)C6)C4)sc23)c1] |
| G116 | 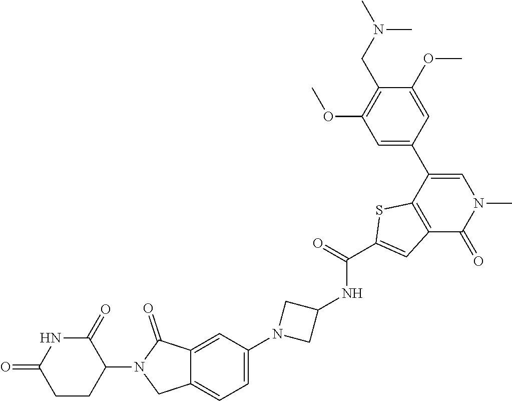 [SMILES: COc1cc(-c2cn(C)c(=O)c3cc(C(=O)NC4CN(c5ccc6c(c5)C(=O)N(C5CCC(=O)NC5=O)C6)C4)sc23)cc(OC)c1CN(C)C] |
| G117 | 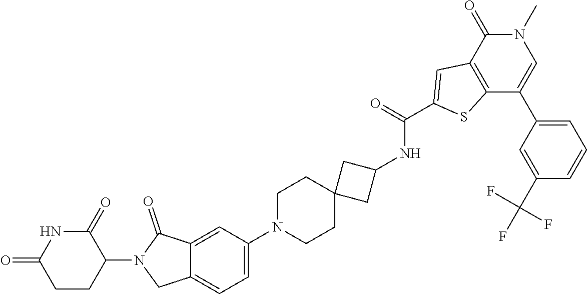 [SMILES: Cn1cc(-c2cccc(C(F)(F)F)c2)c2sc(C(=O)NC3CC4(CCN(c5ccc6c(c5)C(=O)N(C5CCC(=O)NC5=O)C6)CC4)C3)cc2c1=O] |
| G118 | 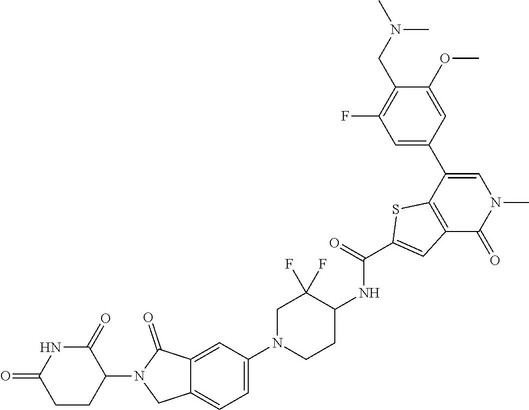 [SMILES: COc1cc(-c2cn(C)c(=O)c3cc(C(=O)NC4CCN(c5ccc6c(c5)C(=O)N(C5CCC(=O)NC5=O)C6)CC4(F)F)sc23)cc(F)c1CN(C)C] |
| G119 | 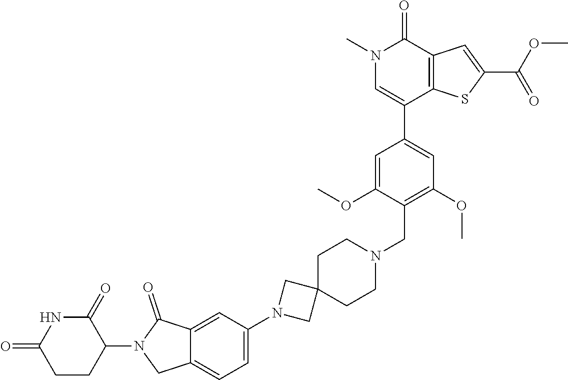 [SMILES: COC(=O)c1cc2c(=O)n(C)cc(-c3cc(OC)c(CN4CCC5(CC4)CN(c4ccc6c(c4)C(=O)N(C4CCC(=O)NC4=O)C6)C5)c(OC)c3)c2s1] |
| G120 | 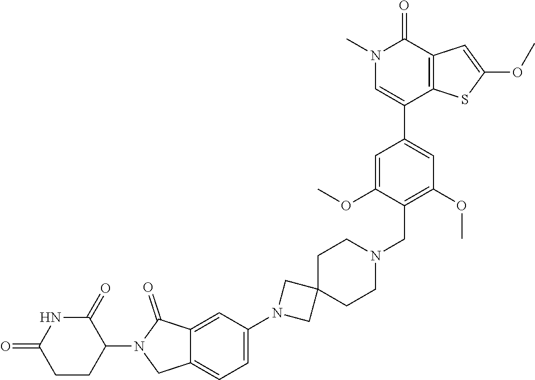 [SMILES: COc1cc2c(=O)n(C)cc(-c3cc(OC)c(CN4CCC5(CC4)CN(c4ccc6c(c4)C(=O)N(C4CCC(=O)NC4=O)C6)C5)c(OC)c3)c2s1] |
| G121 | 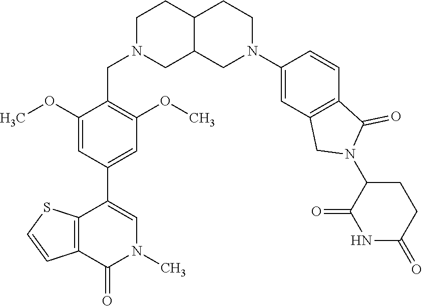 [SMILES: COc1cc(-c2cn(C)c(=O)c3ccsc23)cc(OC)c1CN1CCC2CCN(c3ccc4c(c3)CN(C3CCC(=O)NC3=O)C4=O)CC2C1] |
| G122 | 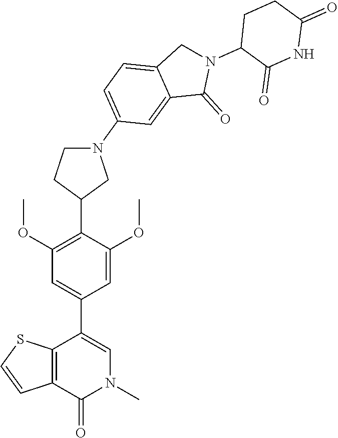 [SMILES: COc1cc(-c2cn(C)c(=O)c3ccsc23)cc(OC)c1C1CCN(c2ccc3c(c2)C(=O)N(C2CCC(=O)NC2=O)C3)C1] |
| G123 | 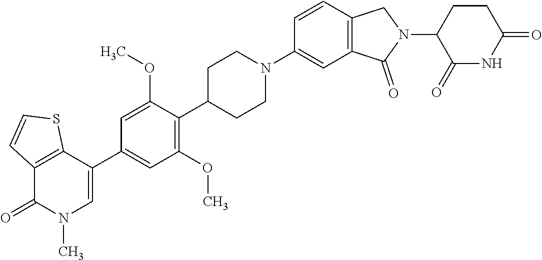 [SMILES: COc1cc(-c2cn(C)c(=O)c3ccsc23)cc(OC)c1C1CCN(c2ccc3c(c2)C(=O)N(C2CCC(=O)NC2=O)C3)CC1] |
| G124 | 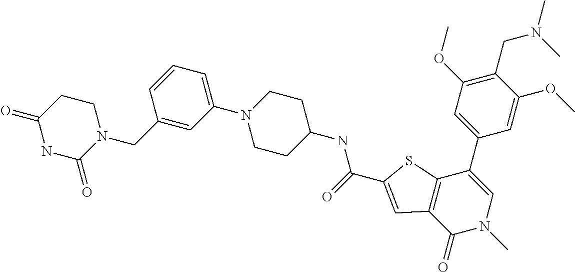 [SMILES: COc1cc(-c2cn(C)c(=O)c3cc(C(=O)NC4CCN(c5cccc(CN6CCC(=O)NC6=O)c5)CC4)sc23)cc(OC)c1CN(C)C] |
| G125 | 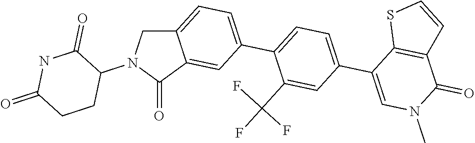 [SMILES: Cn1cc(-c2ccc(-c3ccc4c(c3)C(=O)N(C3CCC(=O)NC3=O)C4)c(C(F)(F)F)c2)c2sccc2c1=O] |
| G126 | 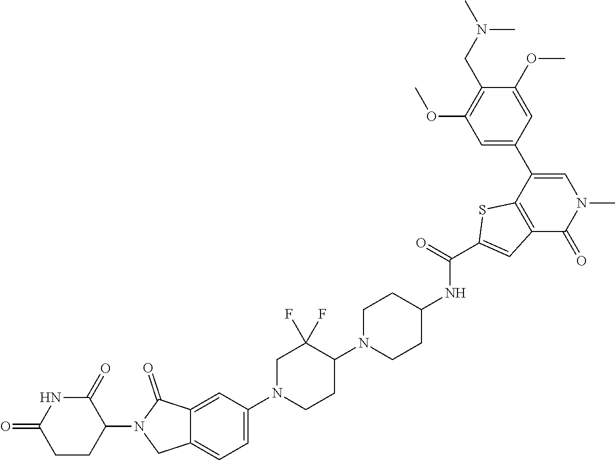 [SMILES: COc1cc(-c2cn(C)c(=O)c3cc(C(=O)NC4CCN(C5CCN(c6ccc7c(c6)C(=O)N(C6CCC(=O)NC6=O)C7)CC5(F)F)CC4)sc23)cc(OC)c1CN(C)C] |
| G127 | 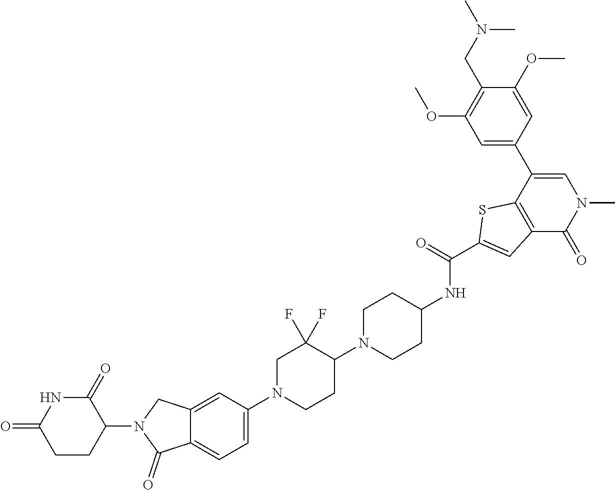 [SMILES: COc1cc(-c2cn(C)c(=O)c3cc(C(=O)NC4CCN(C5CCN(c6ccc7c(c6)CN(C6CCC(=O)NC6=O)C7=O)CC5(F)F)CC4)sc23)cc(OC)c1CN(C)C] |
| G128 | 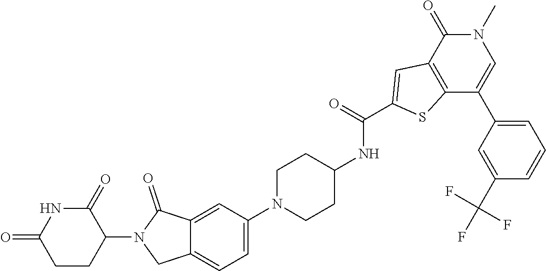 [SMILES: Cn1cc(-c2cccc(C(F)(F)F)c2)c2sc(C(=O)NC3CCN(c4ccc5c(c4)C(=O)N(C4CCC(=O)NC4=O)C5)CC3)cc2c1=O] |
| G129 | 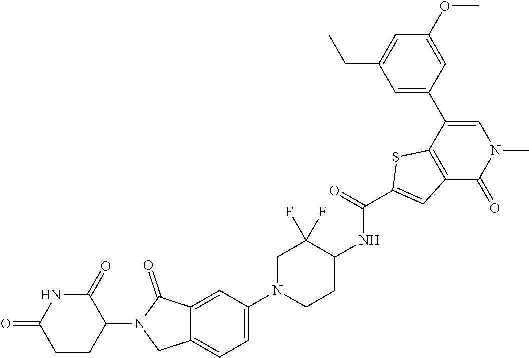 [SMILES: CCc1cc(OC)cc(-c2cn(C)c(=O)c3cc(C(=O)NC4CCN(c5ccc6c(c5)C(=O)N(C5CCC(=O)NC5=O)C6)CC4(F)F)sc23)c1] |
| G130 | 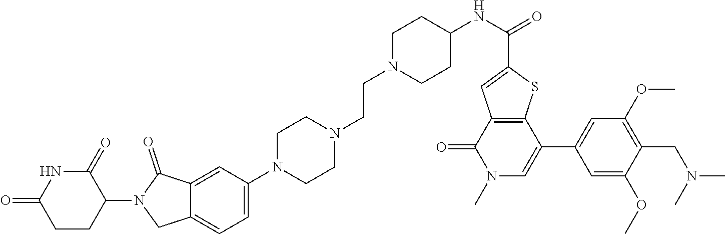 [SMILES: COc1cc(-c2cn(C)c(=O)c3cc(C(=O)NC4CCN(CCN5CCN(c6ccc7c(c6)C(=O)N(C6CCC(=O)NC6=O)C7)CC5)CC4)sc23)cc(OC)c1CN(C)C] |
| G131 | 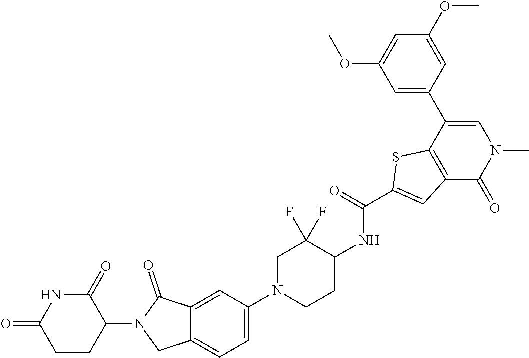 [SMILES: COc1cc(OC)cc(-c2cn(C)c(=O)c3cc(C(=O)NC4CCN(c5ccc6c(c5)C(=O)N(C5CCC(=O)NC5=O)C6)CC4(F)F)sc23)c1] |
| G132 | 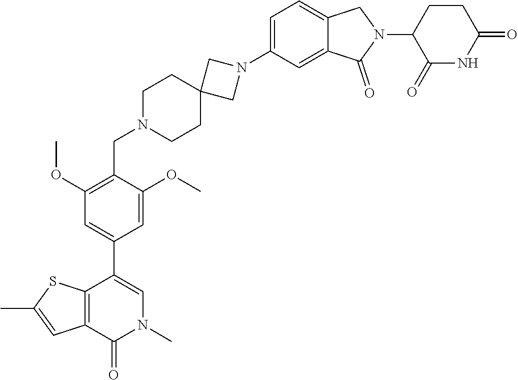 [SMILES: COc1cc(-c2cn(C)c(=O)c3cc(C)sc23)cc(OC)c1CN1CCC2(CC1)CN(c1ccc3c(c1)C(=O)N(C1CCC(=O)NC1=O)C3)C2] |
| G133 | 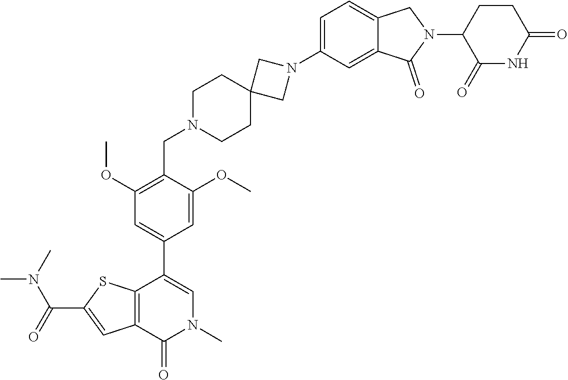 [SMILES: COc1cc(-c2cn(C)c(=O)c3cc(C(=O)N(C)C)sc23)cc(OC)c1CN1CCC2(CC1)CN(c1ccc3c(c1)C(=O)N(C1CCC(=O)NC1=O)C3)C2] |
| G134 | 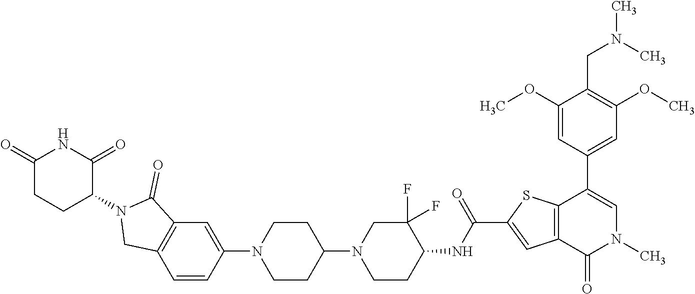 [SMILES: COc1cc(-c2cn(C)c(=O)c3cc(C(=O)N[C@@H]4CCN(C5CCN(c6ccc7c(c6)C(=O)N([C@@H]6CCC(=O)NC6=O)C7)CC5)CC4(F)F)sc23)cc(OC)c1CN(C)C] |
| G135 | 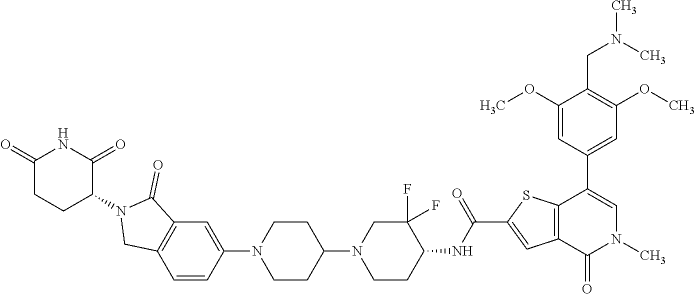 [SMILES: COc1cc(-c2cn(C)c(=O)c3cc(C(=O)N[C@@H]4CCN(C5CCN(c6ccc7c(c6)C(=O)N([C@@H]6CCC(=O)NC6=O)C7)CC5)CC4(F)F)sc23)cc(OC)c1CN(C)C] |
| G136 | 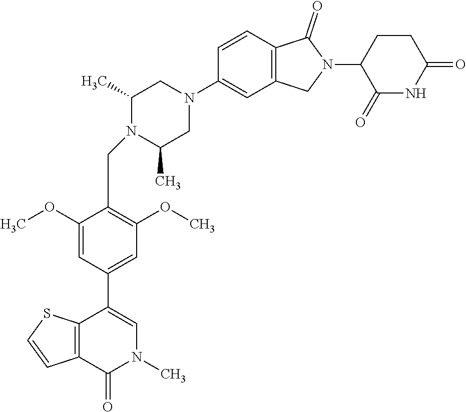 [SMILES: COc1cc(-c2cn(C)c(=O)c3ccsc23)cc(OC)c1CN1[C@H](C)CN(c2ccc3c(c2)CN(C2CCC(=O)NC2=O)C3=O)C[C@H]1C] |
| G137 | 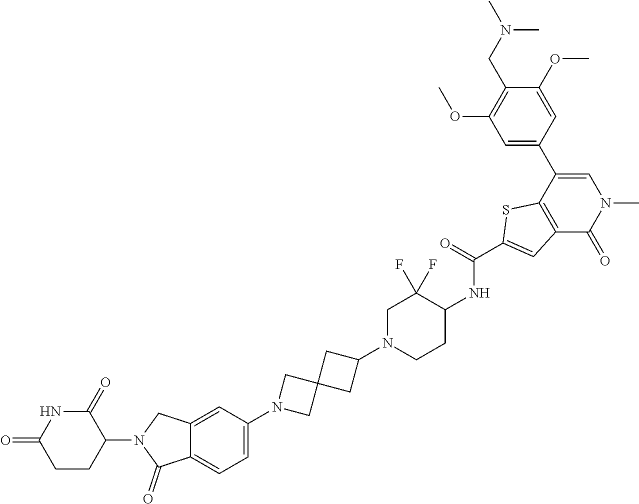 [SMILES: COc1cc(-c2cn(C)c(=O)c3cc(C(=O)NC4CCN(C5CC6(C5)CN(c5ccc7c(c5)CN(C5CCC(=O)NC5=O)C7=O)C6)CC4(F)F)sc23)cc(OC)c1CN(C)C] |
| G138 | 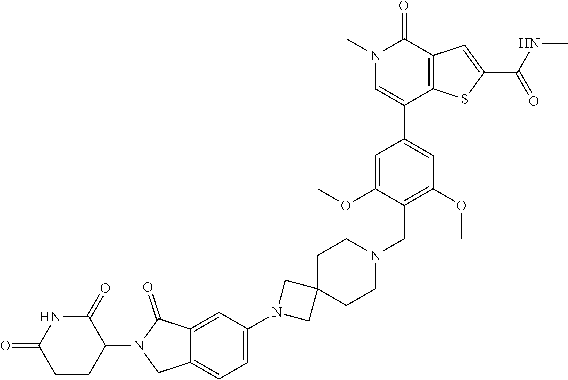 [SMILES: CNC(=O)c1cc2c(=O)n(C)cc(-c3cc(OC)c(CN4CCC5(CC4)CN(c4ccc6c(c4)C(=O)N(C4CCC(=O)NC4=O)C6)C5)c(OC)c3)c2s1] |
| G139 | 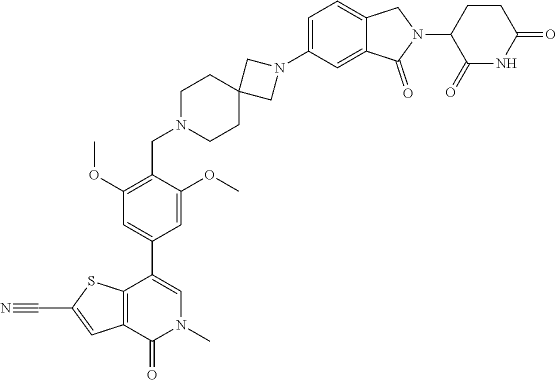 [SMILES: COc1cc(-c2cn(C)c(=O)c3cc(C#N)sc23)cc(OC)c1CN1CCC2(CC1)CN(c1ccc3c(c1)C(=O)N(C1CCC(=O)NC1=O)C3)C2] |
| G140 | 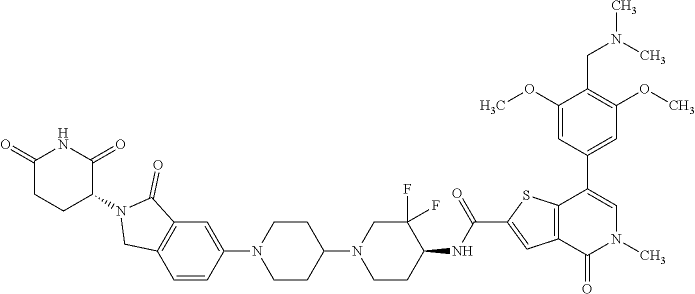 [SMILES: COc1cc(-c2cn(C)c(=O)c3cc(C(=O)N[C@H]4CCN(C5CCN(c6ccc7c(c6)C(=O)N([C@@H]6CCC(=O)NC6=O)C7)CC5)CC4(F)F)sc23)cc(OC)c1CN(C)C] |
| G141 | 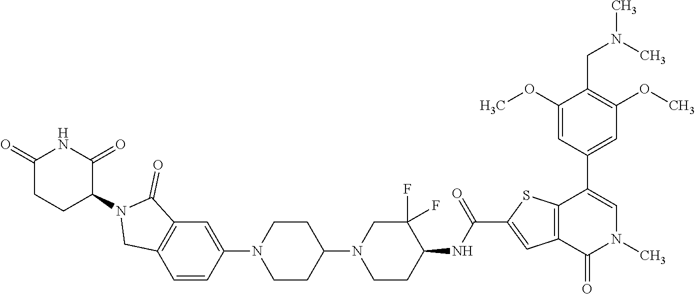 [SMILES: COc1cc(-c2cn(C)c(=O)c3cc(C(=O)N[C@H]4CCN(C5CCN(c6ccc7c(c6)C(=O)N([C@H]6CCC(=O)NC6=O)C7)CC5)CC4(F)F)sc23)cc(OC)c1CN(C)C] |
| G142 | 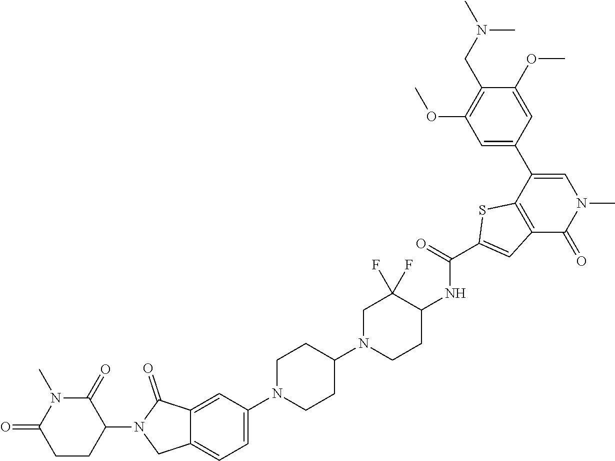 [SMILES: COc1cc(-c2cn(C)c(=O)c3cc(C(=O)NC4CCN(C5CCN(c6ccc7c(c6)C(=O)N(C6CCC(=O)N(C)C6=O)C7)CC5)CC4(F)F)sc23)cc(OC)c1CN(C)C] |
| G143 | 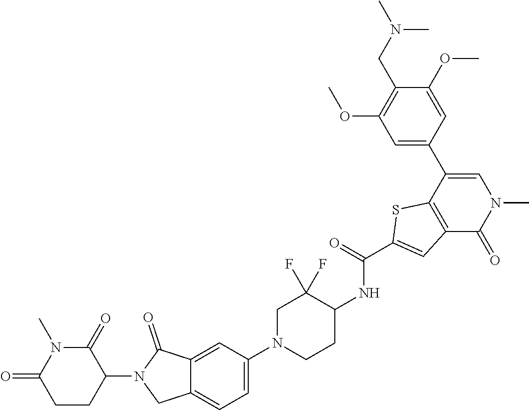 [SMILES: COc1cc(-c2cn(C)c(=O)c3cc(C(=O)NC4CCN(c5ccc6c(c5)C(=O)N(C5CCC(=O)N(C)C5=O)C6)CC4(F)F)sc23)cc(OC)c1CN(C)C] |
| G144 | 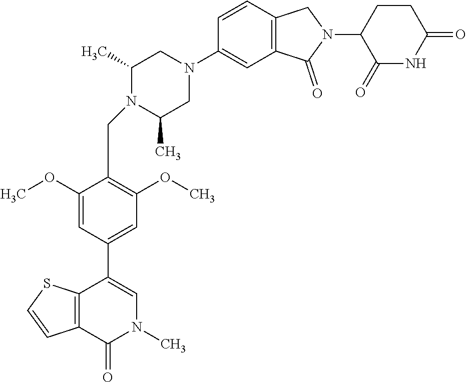 [SMILES: COc1cc(-c2cn(C)c(=O)c3ccsc23)cc(OC)c1CN1[C@H](C)CN(c2ccc3c(c2)C(=O)N(C2CCC(=O)NC2=O)C3)C[C@H]1C] |
| G145 | 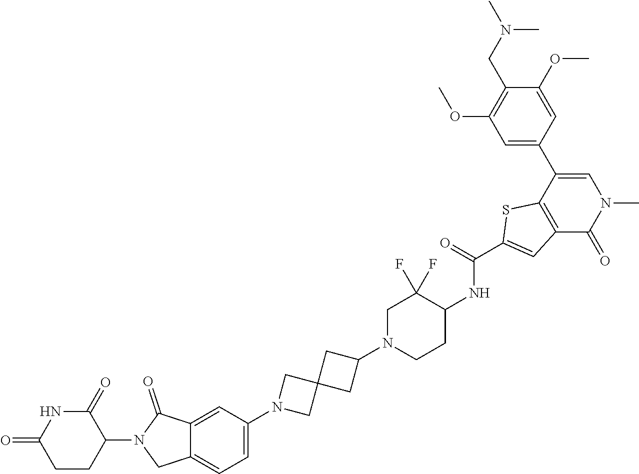 [SMILES: COc1cc(-c2cn(C)c(=O)c3cc(C(=O)NC4CCN(C5CC6(C5)CN(c5ccc7c(c5)C(=O)N(C5CCC(=O)NC5=O)C7)C6)CC4(F)F)sc23)cc(OC)c1CN(C)C] |
| G146 | 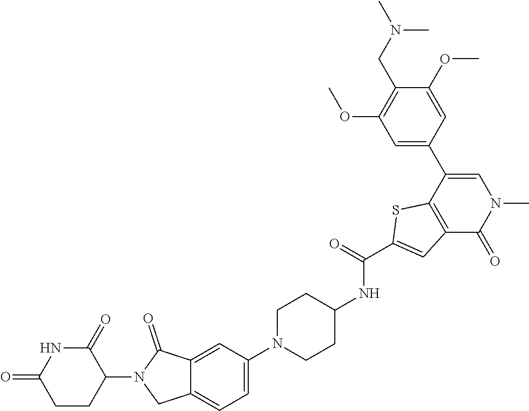 [SMILES: COc1cc(-c2cn(C)c(=O)c3cc(C(=O)NC4CCN(c5ccc6c(c5)C(=O)N(C5CCC(=O)NC5=O)C6)CC4)sc23)cc(OC)c1CN(C)C] |
| G147 | 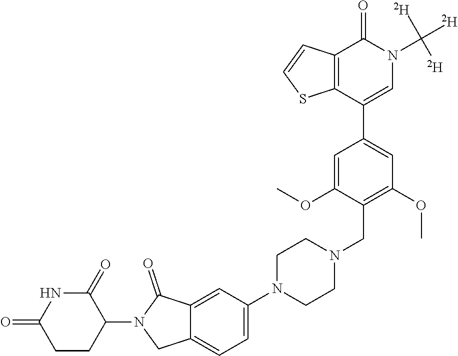 [SMILES: [2H]C([2H])([2H])n1cc(-c2cc(OC)c(CN3CCN(c4ccc5c(c4)C(=O)N(C4CCC(=O)NC4=O)C5)CC3)c(OC)c2)c2sccc2c1=O] |
| G148 | 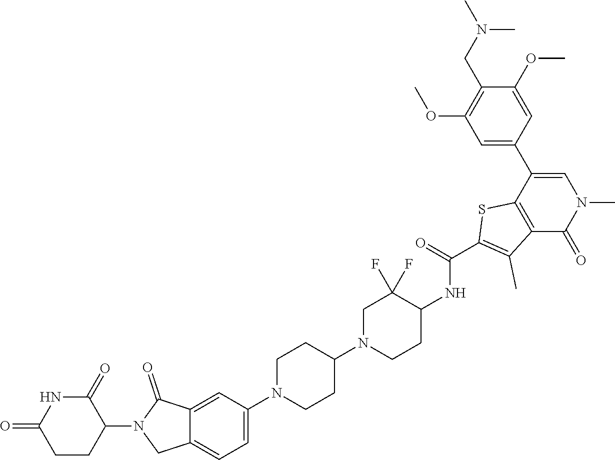 [SMILES: COc1cc(-c2cn(C)c(=O)c3c(C)c(C(=O)NC4CCN(C5CCN(c6ccc7c(c6)C(=O)N(C6CCC(=O)NC6=O)C7)CC5)CC4(F)F)sc23)cc(OC)c1CN(C)C] |
| G149 | 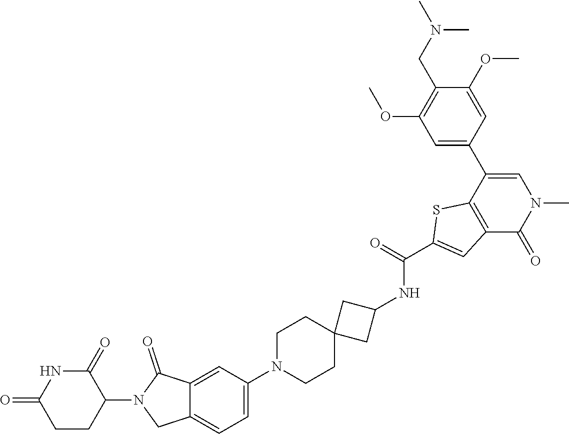 [SMILES: COc1cc(-c2cn(C)c(=O)c3cc(C(=O)NC4CC5(CCN(c6ccc7c(c6)C(=O)N(C6CCC(=O)NC6=O)C7)CC5)C4)sc23)cc(OC)c1CN(C)C] |
| G150 | 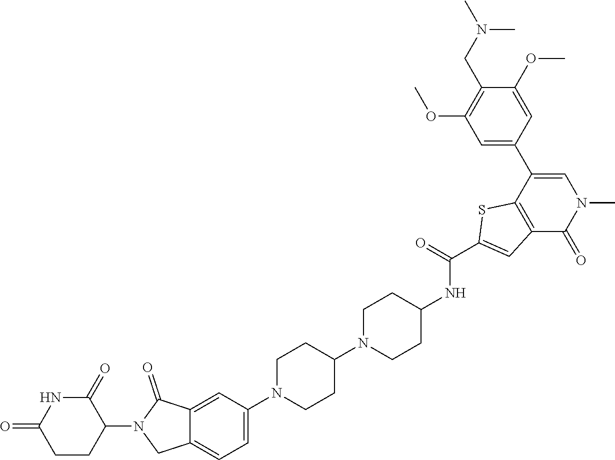 [SMILES: COc1cc(-c2cn(C)c(=O)c3cc(C(=O)NC4CCN(C5CCN(c6ccc7c(c6)C(=O)N(C6CCC(=O)NC6=O)C7)CC5)CC4)sc23)cc(OC)c1CN(C)C] |
| G151 | 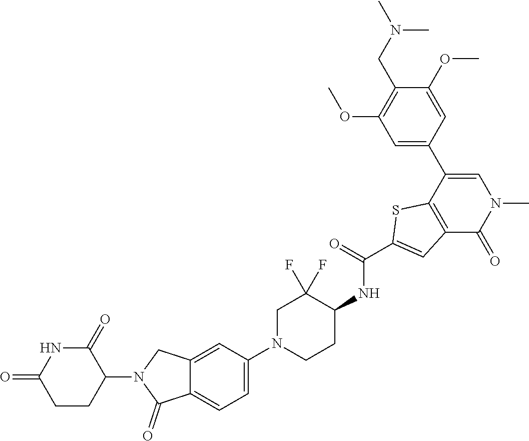 [SMILES: COc1cc(-c2cn(C)c(=O)c3cc(C(=O)N[C@H]4CCN(c5ccc6c(c5)CN(C5CCC(=O)NC5=O)C6=O)CC4(F)F)sc23)cc(OC)c1CN(C)C] |
| G152 | 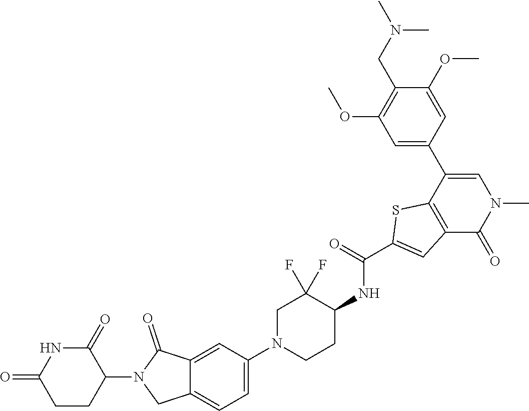 [SMILES: COc1cc(-c2cn(C)c(=O)c3cc(C(=O)N[C@H]4CCN(c5ccc6c(c5)C(=O)N(C5CCC(=O)NC5=O)C6)CC4(F)F)sc23)cc(OC)c1CN(C)C] |
| G153 | 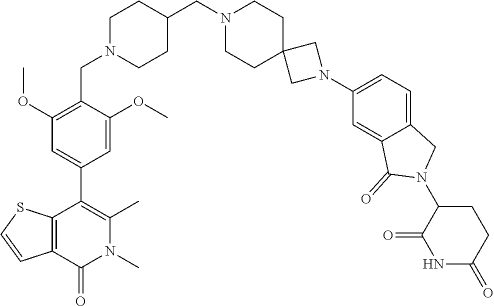 [SMILES: COc1cc(-c2c(C)n(C)c(=O)c3ccsc23)cc(OC)c1CN1CCC(CN2CCC3(CC2)CN(c2ccc4c(c2)C(=O)N(C2CCC(=O)NC2=O)C4)C3)CC1] |
| G154 | 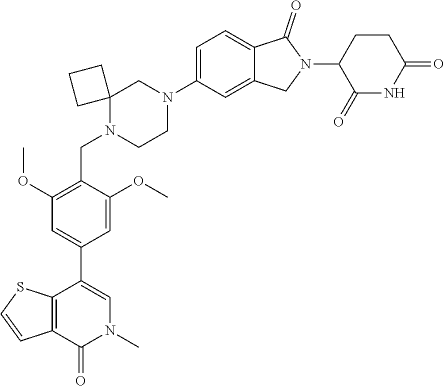 [SMILES: COc1cc(-c2cn(C)c(=O)c3ccsc23)cc(OC)c1CN1CCN(c2ccc3c(c2)CN(C2CCC(=O)NC2=O)C3=O)CC12CCC2] |
| G155 | 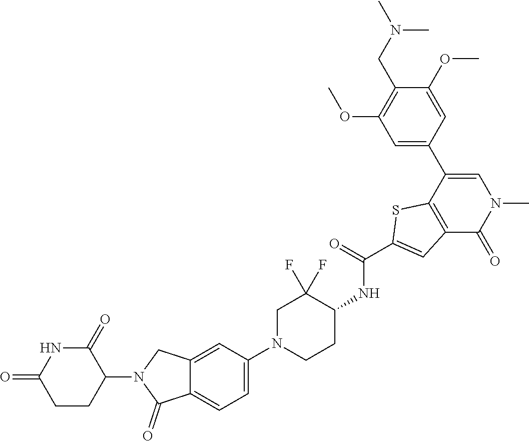 [SMILES: COc1cc(-c2cn(C)c(=O)c3cc(C(=O)N[C@@H]4CCN(c5ccc6c(c5)CN(C5CCC(=O)NC5=O)C6=O)CC4(F)F)sc23)cc(OC)c1CN(C)C] |
| G156 | 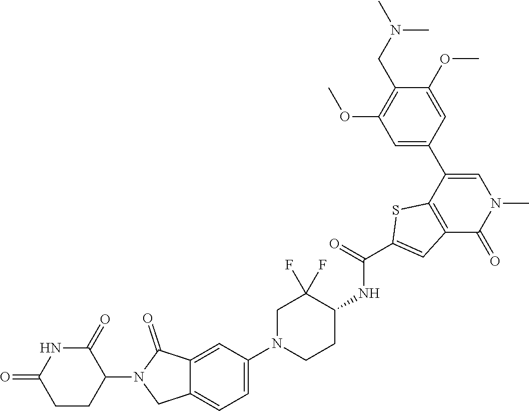 [SMILES: COc1cc(-c2cn(C)c(=O)c3cc(C(=O)N[C@@H]4CCN(c5ccc6c(c5)C(=O)N(C5CCC(=O)NC5=O)C6)CC4(F)F)sc23)cc(OC)c1CN(C)C] |
| G157 | 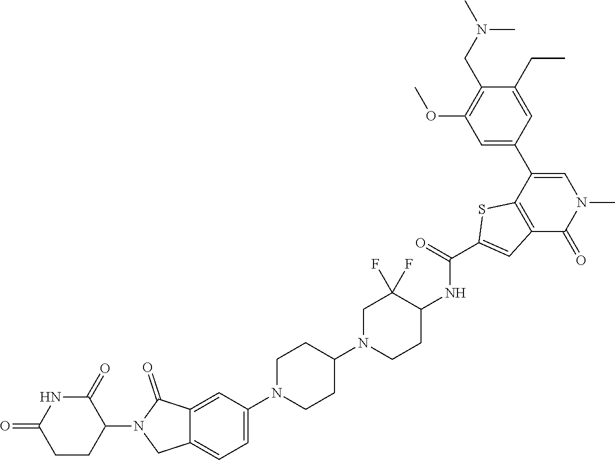 [SMILES: CCc1cc(-c2cn(C)c(=O)c3cc(C(=O)NC4CCN(C5CCN(c6ccc7c(c6)C(=O)N(C6CCC(=O)NC6=O)C7)CC5)CC4(F)F)sc23)cc(OC)c1CN(C)C] |
| G158 | 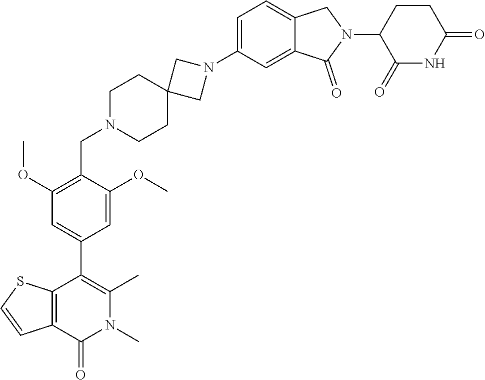 [SMILES: COc1cc(-c2c(C)n(C)c(=O)c3ccsc23)cc(OC)c1CN1CCC2(CC1)CN(c1ccc3c(c1)C(=O)N(C1CCC(=O)NC1=O)C3)C2] |
| G159 | 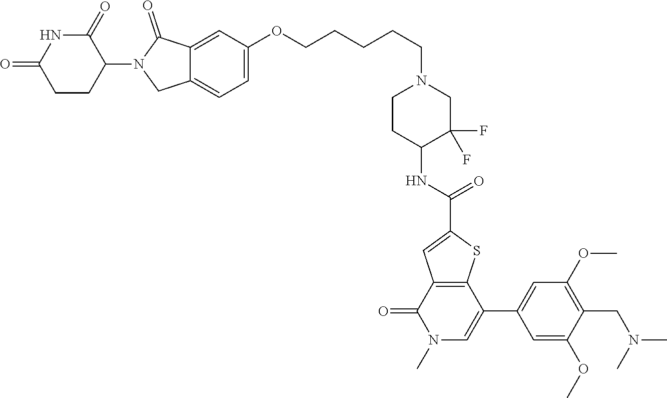 [SMILES: COc1cc(-c2cn(C)c(=O)c3cc(C(=O)NC4CCN(CCCCCOc5ccc6c(c5)C(=O)N(C5CCC(=O)NC5=O)C6)CC4(F)F)sc23)cc(OC)c1CN(C)C] |
| G160 | 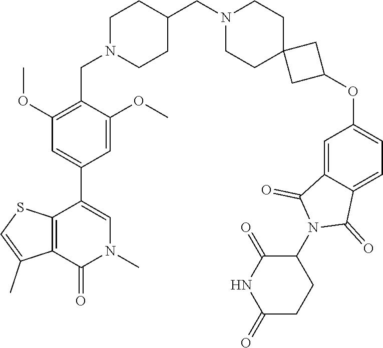 [SMILES: COc1cc(-c2cn(C)c(=O)c3c(C)csc23)cc(OC)c1CN1CCC(CN2CCC3(CC2)CC(Oc2ccc4c(c2)C(=O)N(C2CCC(=O)NC2=O)C4=O)C3)CC1] |
| G161 | 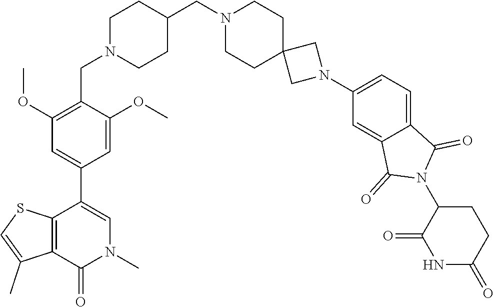 [SMILES: COc1cc(-c2cn(C)c(=O)c3c(C)csc23)cc(OC)c1CN1CCC(CN2CCC3(CC2)CN(c2ccc4c(c2)C(=O)N(C2CCC(=O)NC2=O)C4=O)C3)CC1] |
| G162 | 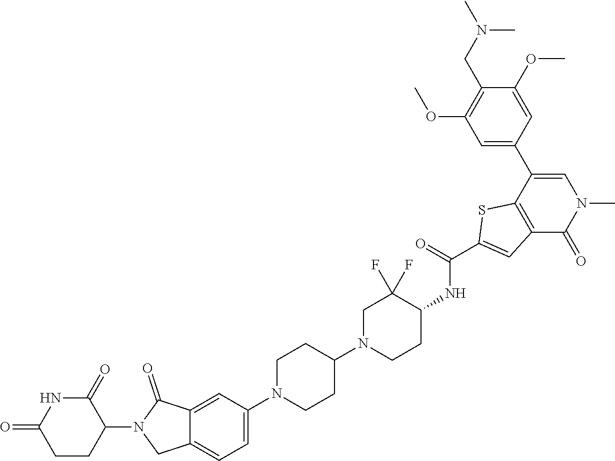 [SMILES: COc1cc(-c2cn(C)c(=O)c3cc(C(=O)N[C@@H]4CCN(C5CCN(c6ccc7c(c6)C(=O)N(C6CCC(=O)NC6=O)C7)CC5)CC4(F)F)sc23)cc(OC)c1CN(C)C] |
| G163 | 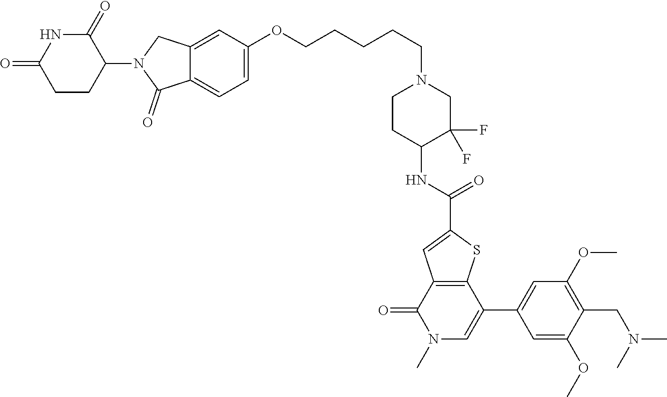 [SMILES: COc1cc(-c2cn(C)c(=O)c3cc(C(=O)NC4CCN(CCCCCOc5ccc6c(c5)CN(C5CCC(=O)NC5=O)C6=O)CC4(F)F)sc23)cc(OC)c1CN(C)C] |
| G164 | 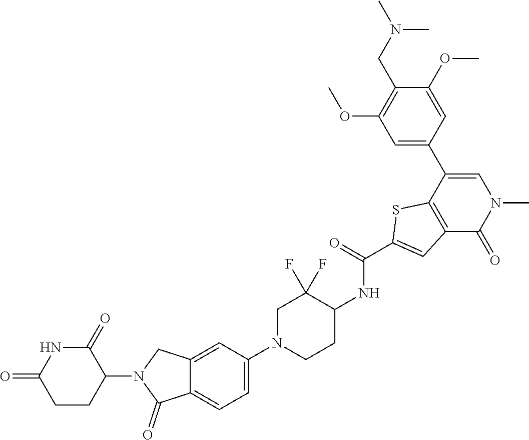 [SMILES: COc1cc(-c2cn(C)c(=O)c3cc(C(=O)NC4CCN(c5ccc6c(c5)CN(C5CCC(=O)NC5=O)C6=O)CC4(F)F)sc23)cc(OC)c1CN(C)C] |
| G165 | 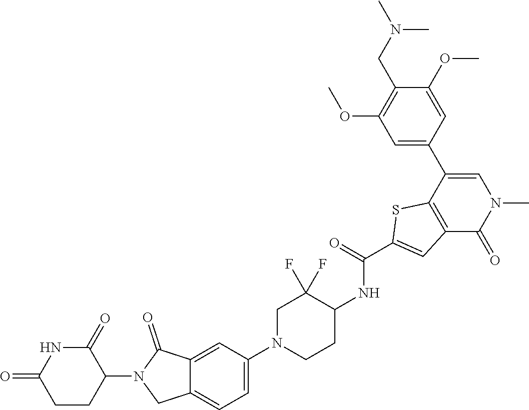 [SMILES: COc1cc(-c2cn(C)c(=O)c3cc(C(=O)NC4CCN(c5ccc6c(c5)C(=O)N(C5CCC(=O)NC5=O)C6)CC4(F)F)sc23)cc(OC)c1CN(C)C] |
| G166 | 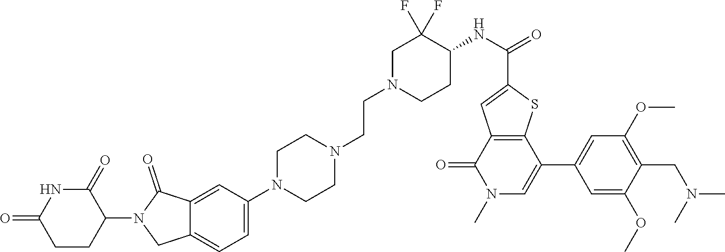 [SMILES: COc1cc(-c2cn(C)c(=O)c3cc(C(=O)N[C@@H]4CCN(CCN5CCN(c6ccc7c(c6)C(=O)N(C6CCC(=O)NC6=O)C7)CC5)CC4(F)F)sc23)cc(OC)c1CN(C)C] |
| G167 | 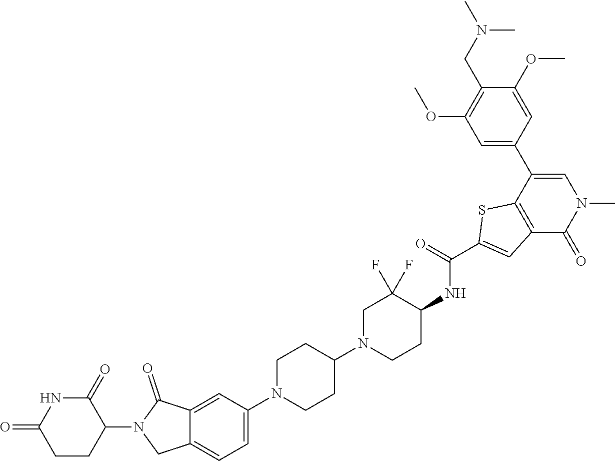 [SMILES: COc1cc(-c2cn(C)c(=O)c3cc(C(=O)N[C@H]4CCN(C5CCN(c6ccc7c(c6)C(=O)N(C6CCC(=O)NC6=O)C7)CC5)CC4(F)F)sc23)cc(OC)c1CN(C)C] |
| G168 | 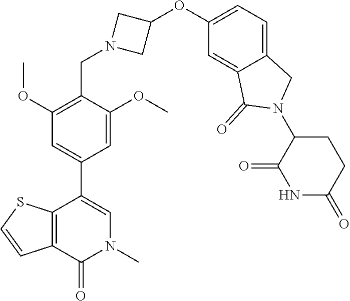 [SMILES: COc1cc(-c2cn(C)c(=O)c3ccsc23)cc(OC)c1CN1CC(Oc2ccc3c(c2)C(=O)N(C2CCC(=O)NC2=O)C3)C1] |
| G169 | 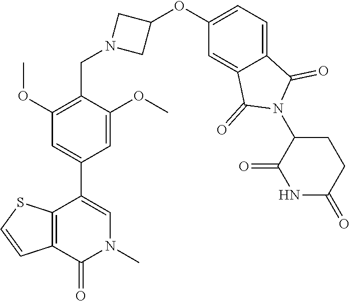 [SMILES: COc1cc(-c2cn(C)c(=O)c3ccsc23)cc(OC)c1CN1CC(Oc2ccc3c(c2)C(=O)N(C2CCC(=O)NC2=O)C3=O)C1] |
| G170 | 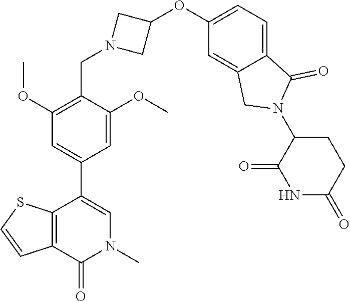 [SMILES: COc1cc(-c2cn(C)c(=O)c3ccsc23)cc(OC)c1CN1CC(Oc2ccc3c(c2)CN(C2CCC(=O)NC2=O)C3=O)C1] |
| G171 | 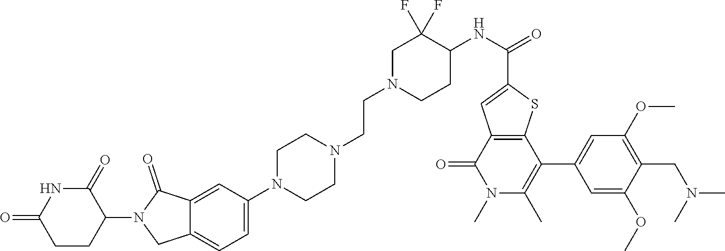 [SMILES: COc1cc(-c2c(C)n(C)c(=O)c3cc(C(=O)NC4CCN(CCN5CCN(c6ccc7c(c6)C(=O)N(C6CCC(=O)NC6=O)C7)CC5)CC4(F)F)sc23)cc(OC)c1CN(C)C] |
| G172 | 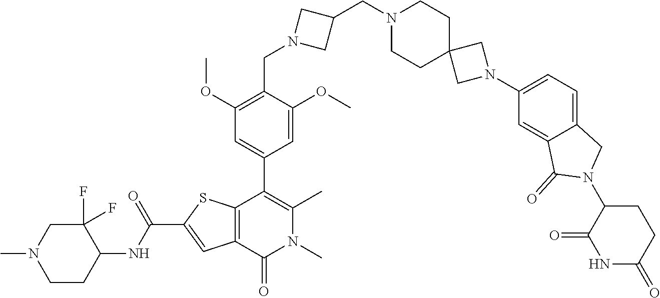 [SMILES: COc1cc(-c2c(C)n(C)c(=O)c3cc(C(=O)NC4CCN(C)CC4(F)F)sc23)cc(OC)c1CN1CC(CN2CCC3(CC2)CN(c2ccc4c(c2)C(=O)N(C2CCC(=O)NC2=O)C4)C3)C1] |
| G173 | 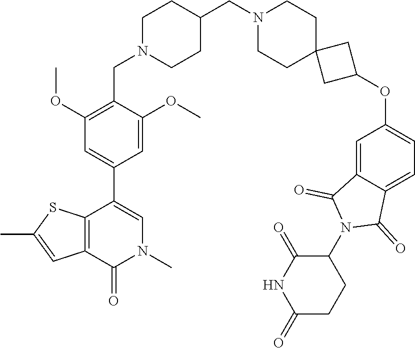 [SMILES: COc1cc(-c2cn(C)c(=O)c3cc(C)sc23)cc(OC)c1CN1CCC(CN2CCC3(CC2)CC(Oc2ccc4c(c2)C(=O)N(C2CCC(=O)NC2=O)C4=O)C3)CC1] |
| G174 | 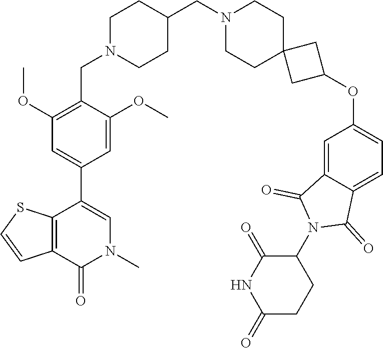 [SMILES: COc1cc(-c2cn(C)c(=O)c3ccsc23)cc(OC)c1CN1CCC(CN2CCC3(CC2)CC(Oc2ccc4c(c2)C(=O)N(C2CCC(=O)NC2=O)C4=O)C3)CC1] |
| G175 | 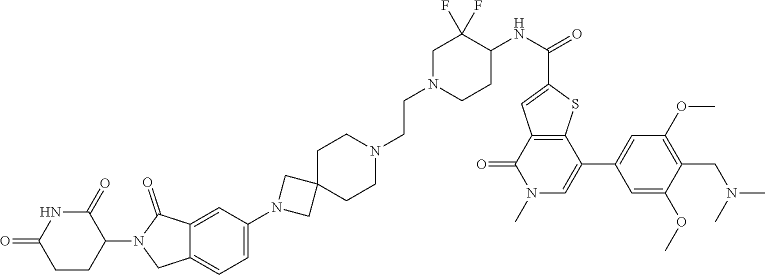 [SMILES: COc1cc(-c2cn(C)c(=O)c3cc(C(=O)NC4CCN(CCN5CCC6(CC5)CN(c5ccc7c(c5)C(=O)N(C5CCC(=O)NC5=O)C7)C6)CC4(F)F)sc23)cc(OC)c1CN(C)C] |
| G176 | 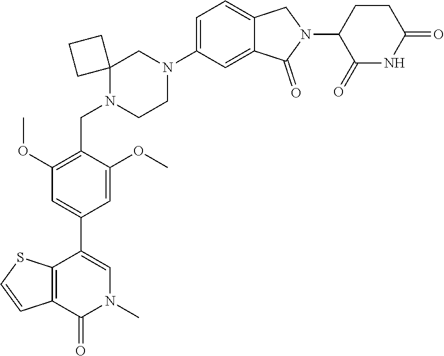 [SMILES: COc1cc(-c2cn(C)c(=O)c3ccsc23)cc(OC)c1CN1CCN(c2ccc3c(c2)C(=O)N(C2CCC(=O)NC2=O)C3)CC12CCC2] |
| G177 | 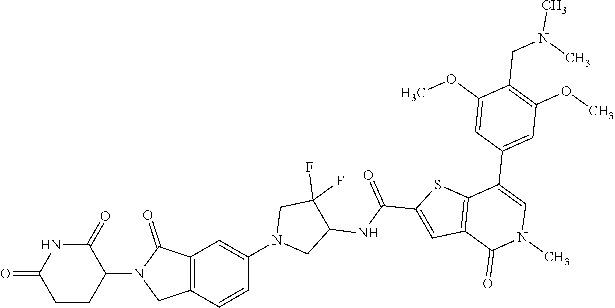 [SMILES: COc1cc(-c2cn(C)c(=O)c3cc(C(=O)NC4CN(c5ccc6c(c5)C(=O)N(C5CCC(=O)NC5=O)C6)CC4(F)F)sc23)cc(OC)c1CN(C)C] |
| G178 | 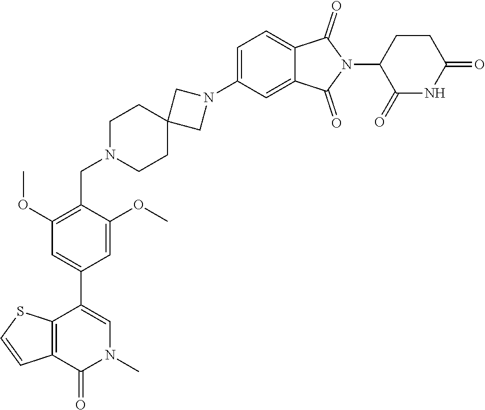 [SMILES: COc1cc(-c2cn(C)c(=O)c3ccsc23)cc(OC)c1CN1CCC2(CC1)CN(c1ccc3c(c1)C(=O)N(C1CCC(=O)NC1=O)C3=O)C2] |
| G179 | 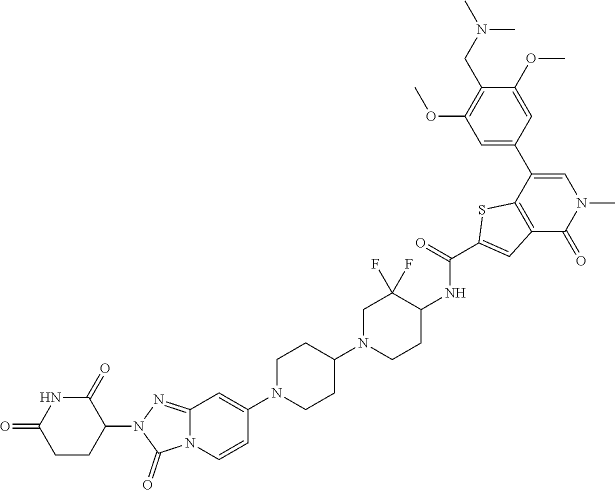 [SMILES: COc1cc(-c2cn(C)c(=O)c3cc(C(=O)NC4CCN(C5CCN(c6ccn7c(=O)n(C8CCC(=O)NC8=O)nc7c6)CC5)CC4(F)F)sc23)cc(OC)c1CN(C)C] |
| G180 | 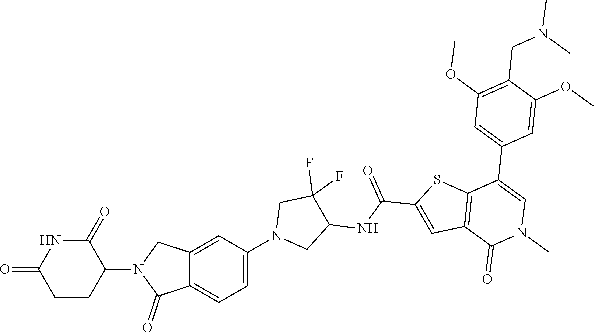 [SMILES: COc1cc(-c2cn(C)c(=O)c3cc(C(=O)NC4CN(c5ccc6c(c5)CN(C5CCC(=O)NC5=O)C6=O)CC4(F)F)sc23)cc(OC)c1CN(C)C] |
| G181 | 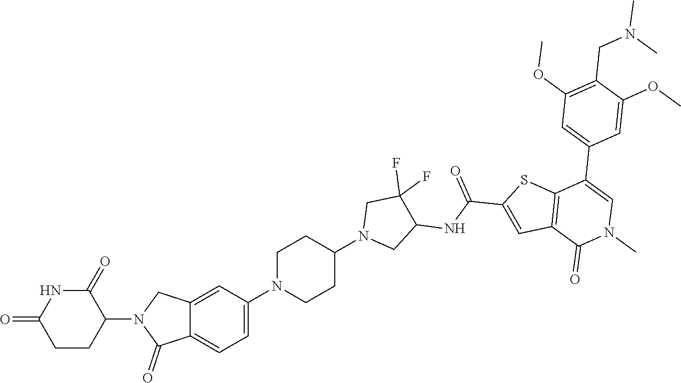 [SMILES: COc1cc(-c2cn(C)c(=O)c3cc(C(=O)NC4CN(C5CCN(c6ccc7c(c6)CN(C6CCC(=O)NC6=O)C7=O)CC5)CC4(F)F)sc23)cc(OC)c1CN(C)C] |
| G182 | 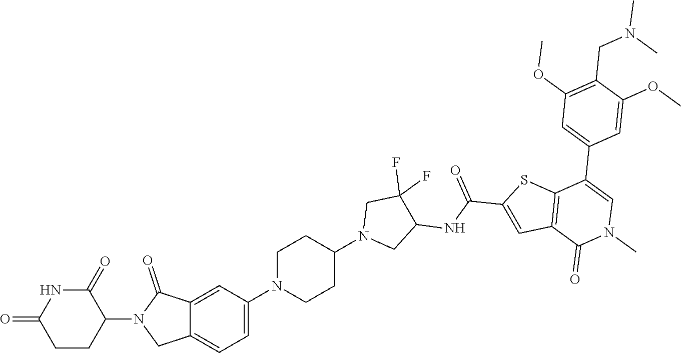 [SMILES: COc1cc(-c2cn(C)c(=O)c3cc(C(=O)NC4CN(C5CCN(c6ccc7c(c6)C(=O)N(C6CCC(=O)NC6=O)C7)CC5)CC4(F)F)sc23)cc(OC)c1CN(C)C] |
| G183 | 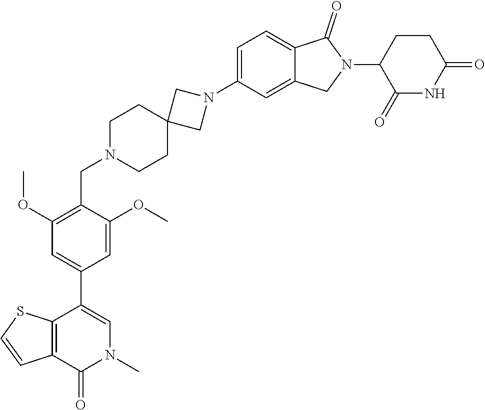 [SMILES: COc1cc(-c2cn(C)c(=O)c3ccsc23)cc(OC)c1CN1CCC2(CC1)CN(c1ccc3c(c1)CN(C1CCC(=O)NC1=O)C3=O)C2] |
| G184 | 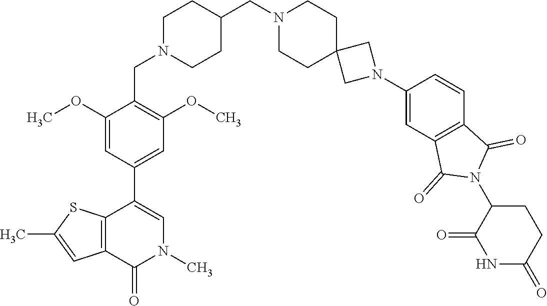 [SMILES: COc1cc(-c2cn(C)c(=O)c3cc(C)sc23)cc(OC)c1CN1CCC(CN2CCC3(CC2)CN(c2ccc4c(c2)C(=O)N(C2CCC(=O)NC2=O)C4=O)C3)CC1] |
| G185 | 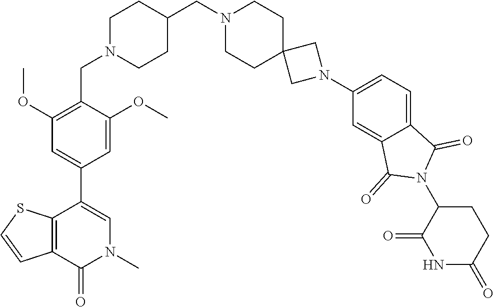 [SMILES: COc1cc(-c2cn(C)c(=O)c3ccsc23)cc(OC)c1CN1CCC(CN2CCC3(CC2)CN(c2ccc4c(c2)C(=O)N(C2CCC(=O)NC2=O)C4=O)C3)CC1] |
| G186 | 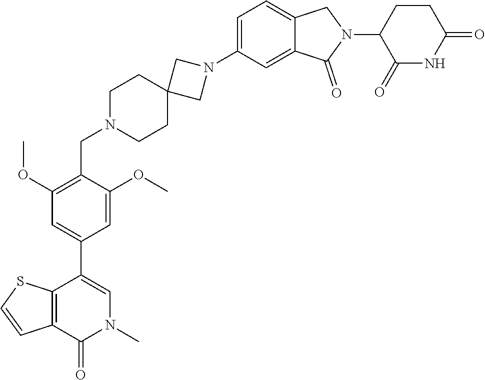 [SMILES: COc1cc(-c2cn(C)c(=O)c3ccsc23)cc(OC)c1CN1CCC2(CC1)CN(c1ccc3c(c1)C(=O)N(C1CCC(=O)NC1=O)C3)C2] |
| G187 | 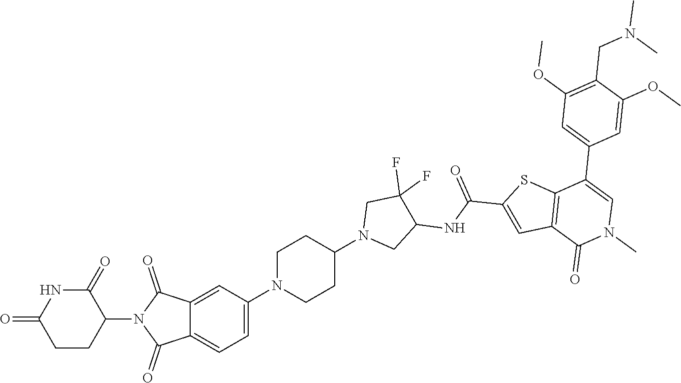 [SMILES: COc1cc(-c2cn(C)c(=O)c3cc(C(=O)NC4CN(C5CCN(c6ccc7c(c6)C(=O)N(C6CCC(=O)NC6=O)C7=O)CC5)CC4(F)F)sc23)cc(OC)c1CN(C)C] |
| G188 | 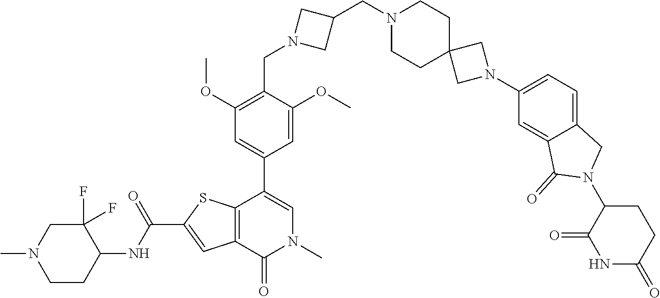 [SMILES: COc1cc(-c2cn(C)c(=O)c3cc(C(=O)NC4CCN(C)CC4(F)F)sc23)cc(OC)c1CN1CC(CN2CCC3(CC2)CN(c2ccc4c(c2)C(=O)N(C2CCC(=O)NC2=O)C4)C3)C1] |
| G189 | 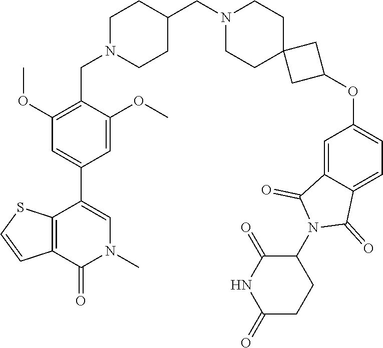 [SMILES: COc1cc(-c2cn(C)c(=O)c3ccsc23)cc(OC)c1CN1CCC(CN2CCC3(CC2)CC(Oc2ccc4c(c2)C(=O)N(C2CCC(=O)NC2=O)C4=O)C3)CC1] |
| G190 | 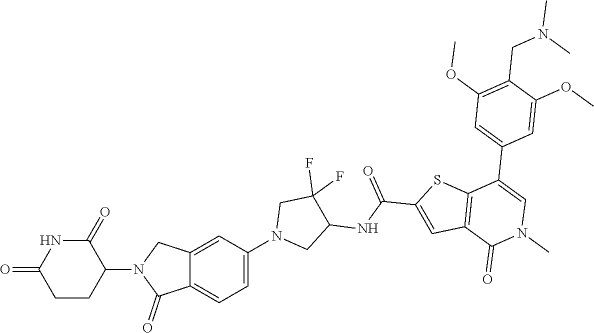 [SMILES: COc1cc(-c2cn(C)c(=O)c3cc(C(=O)NC4CN(c5ccc6c(c5)CN(C5CCC(=O)NC5=O)C6=O)CC4(F)F)sc23)cc(OC)c1CN(C)C] |
| G191 | 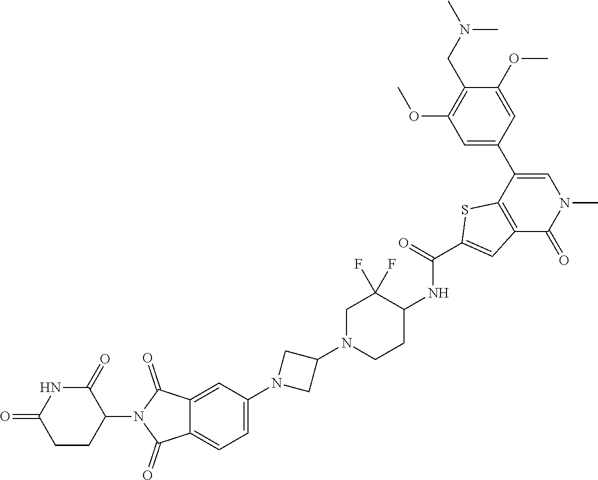 [SMILES: COc1cc(-c2cn(C)c(=O)c3cc(C(=O)NC4CCN(C5CN(c6ccc7c(c6)C(=O)N(C6CCC(=O)NC6=O)C7=O)C5)CC4(F)F)sc23)cc(OC)c1CN(C)C] |
| G192 | 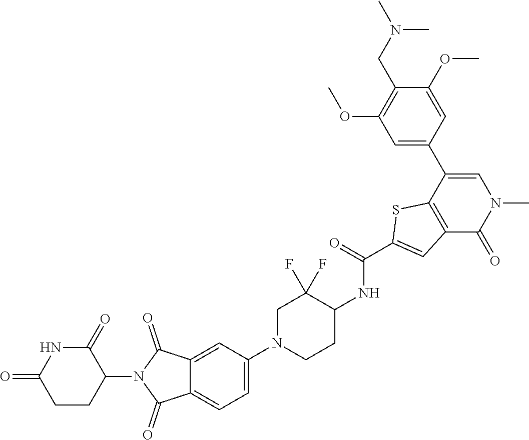 [SMILES: COc1cc(-c2cn(C)c(=O)c3cc(C(=O)NC4CCN(c5ccc6c(c5)C(=O)N(C5CCC(=O)NC5=O)C6=O)CC4(F)F)sc23)cc(OC)c1CN(C)C] |
| G193 | 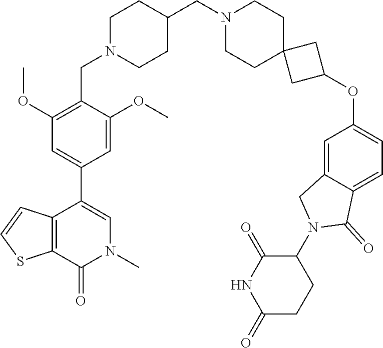 [SMILES: COc1cc(-c2cn(C)c(=O)c3sccc23)cc(OC)c1CN1CCC(CN2CCC3(CC2)CC(Oc2ccc4c(c2)CN(C2CCC(=O)NC2=O)C4=O)C3)CC1] |
| G194 | 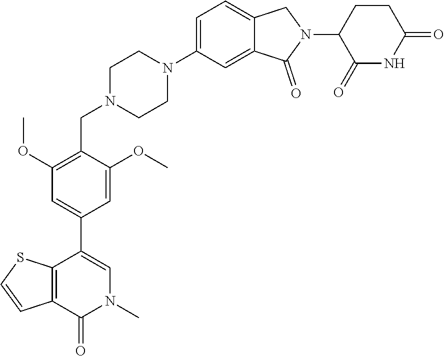 [SMILES: COc1cc(-c2cn(C)c(=O)c3ccsc23)cc(OC)c1CN1CCN(c2ccc3c(c2)C(=O)N(C2CCC(=O)NC2=O)C3)CC1] |
| G195 | 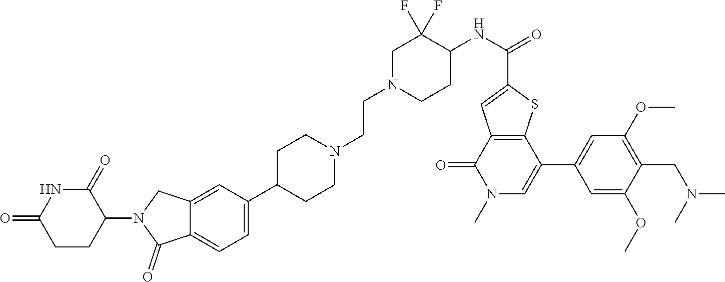 [SMILES: COc1cc(-c2cn(C)c(=O)c3cc(C(=O)NC4CCN(CCN5CCC(c6ccc7c(c6)CN(C6CCC(=O)NC6=O)C7=O)CC5)CC4(F)F)sc23)cc(OC)c1CN(C)C] |
| G196 | 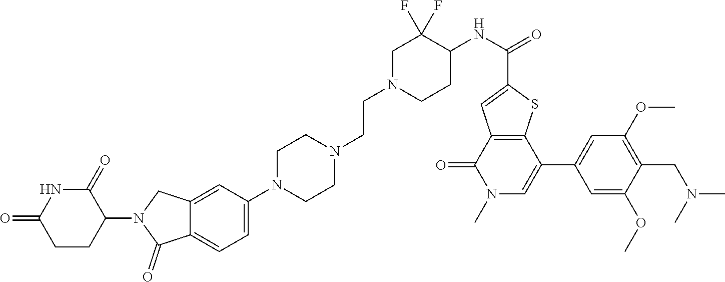 [SMILES: COc1cc(-c2cn(C)c(=O)c3cc(C(=O)NC4CCN(CCN5CCN(c6ccc7c(c6)CN(C6CCC(=O)NC6=O)C7=O)CC5)CC4(F)F)sc23)cc(OC)c1CN(C)C] |
| G197 | 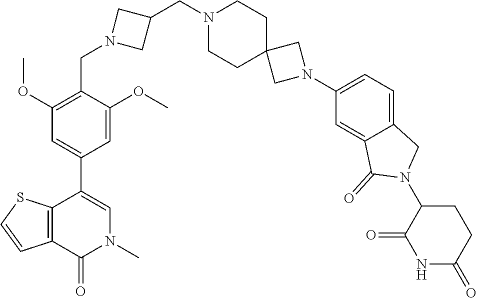 [SMILES: COc1cc(-c2cn(C)c(=O)c3ccsc23)cc(OC)c1CN1CC(CN2CCC3(CC2)CN(c2ccc4c(c2)C(=O)N(C2CCC(=O)NC2=O)C4)C3)C1] |
| G198 | 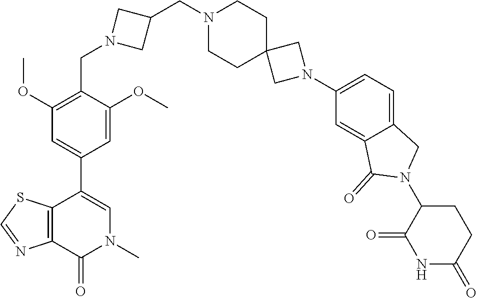 [SMILES: COc1cc(-c2cn(C)c(=O)c3ncsc23)cc(OC)c1CN1CC(CN2CCC3(CC2)CN(c2ccc4c(c2)C(=O)N(C2CCC(=O)NC2=O)C4)C3)C1] |
| G199 | 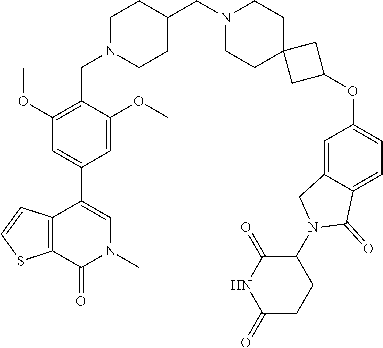 [SMILES: COc1cc(-c2cn(C)c(=O)c3sccc23)cc(OC)c1CN1CCC(CN2CCC3(CC2)CC(Oc2ccc4c(c2)CN(C2CCC(=O)NC2=O)C4=O)C3)CC1] |
| G200 | 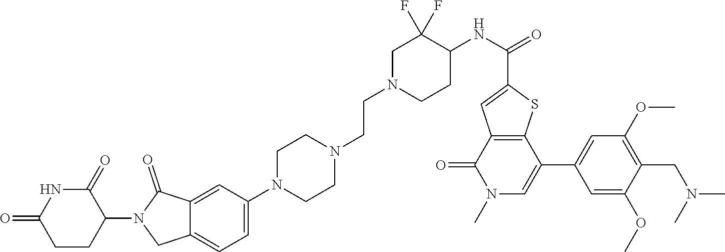 [SMILES: COc1cc(-c2cn(C)c(=O)c3cc(C(=O)NC4CCN(CCN5CCN(c6ccc7c(c6)C(=O)N(C6CCC(=O)NC6=O)C7)CC5)CC4(F)F)sc23)cc(OC)c1CN(C)C] |
| G201 | 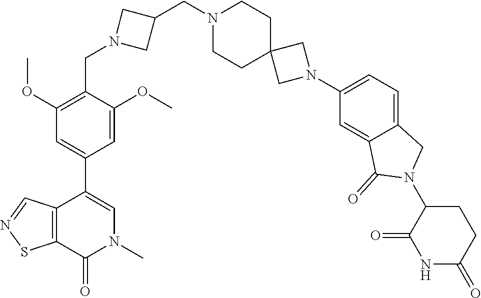 [SMILES: COc1cc(-c2cn(C)c(=O)c3sncc23)cc(OC)c1CN1CC(CN2CCC3(CC2)CN(c2ccc4c(c2)C(=O)N(C2CCC(=O)NC2=O)C4)C3)C1] |
| G202 | 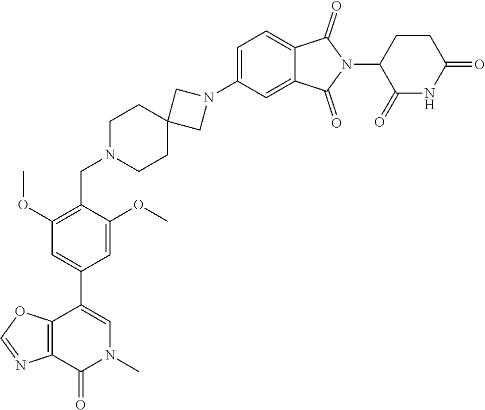 [SMILES: COc1cc(-c2cn(C)c(=O)c3ncoc23)cc(OC)c1CN1CCC2(CC1)CN(c1ccc3c(c1)C(=O)N(C1CCC(=O)NC1=O)C3=O)C2] |
| G203 | 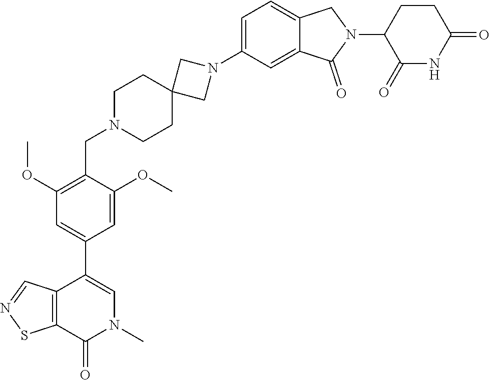 [SMILES: COc1cc(-c2cn(C)c(=O)c3sncc23)cc(OC)c1CN1CCC2(CC1)CN(c1ccc3c(c1)C(=O)N(C1CCC(=O)NC1=O)C3)C2] |
| G204 | 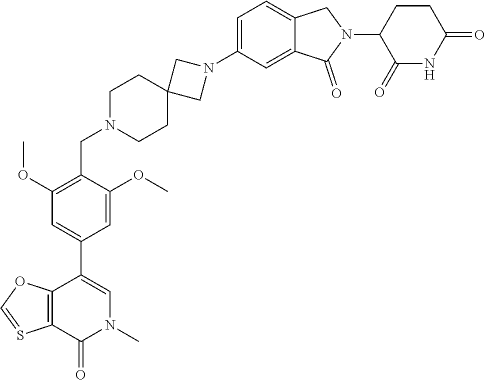 |
| G205 | 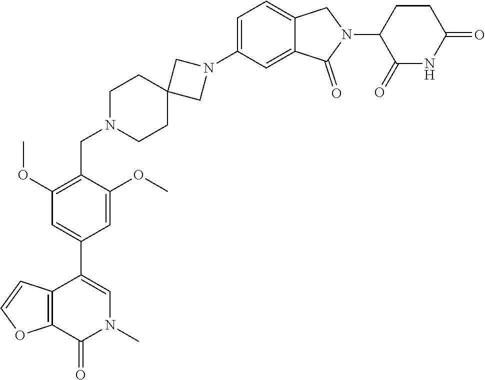 [SMILES: COc1cc(-c2cn(C)c(=O)c3occc23)cc(OC)c1CN1CCC2(CC1)CN(c1ccc3c(c1)C(=O)N(C1CCC(=O)NC1=O)C3)C2] |
| G206 | 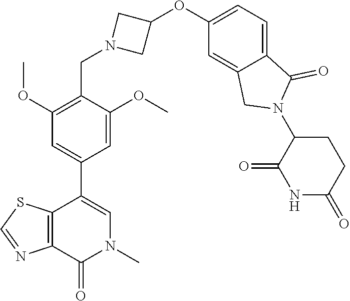 [SMILES: COc1cc(-c2cn(C)c(=O)c3ncsc23)cc(OC)c1CN1CC(Oc2ccc3c(c2)CN(C2CCC(=O)NC2=O)C3=O)C1] |
| G207 | 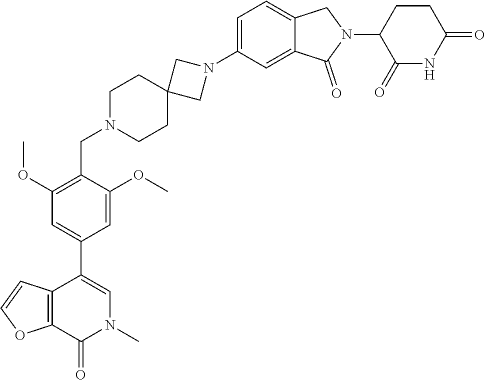 [SMILES: COc1cc(-c2cn(C)c(=O)c3occc23)cc(OC)c1CN1CCC2(CC1)CN(c1ccc3c(c1)C(=O)N(C1CCC(=O)NC1=O)C3)C2] |
| G208 | 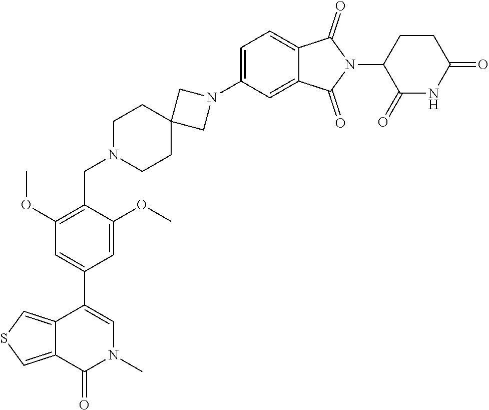 [SMILES: COc1cc(-c2cn(C)c(=O)c3cscc23)cc(OC)c1CN1CCC2(CC1)CN(c1ccc3c(c1)C(=O)N(C1CCC(=O)NC1=O)C3=O)C2] |
| G209 | 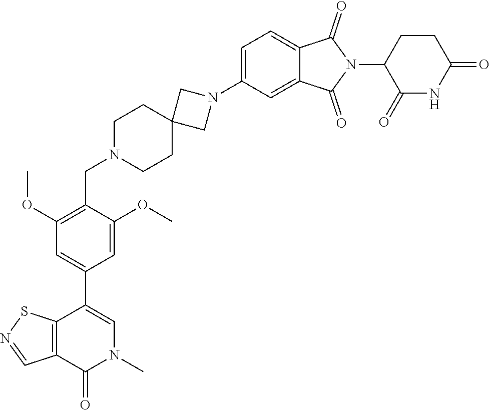 [SMILES: COc1cc(-c2cn(C)c(=O)c3cnsc23)cc(OC)c1CN1CCC2(CC1)CN(c1ccc3c(c1)C(=O)N(C1CCC(=O)NC1=O)C3=O)C2] |
| G210 | 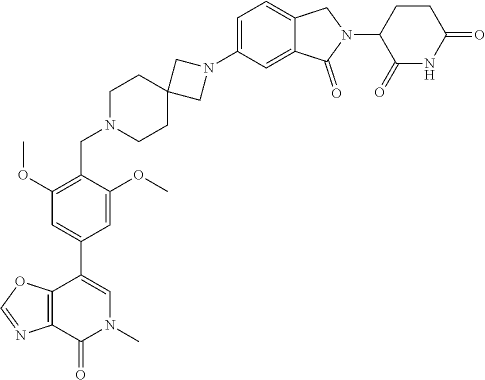 [SMILES: COc1cc(-c2cn(C)c(=O)c3ncoc23)cc(OC)c1CN1CCC2(CC1)CN(c1ccc3c(c1)C(=O)N(C1CCC(=O)NC1=O)C3)C2] |
| G211 | 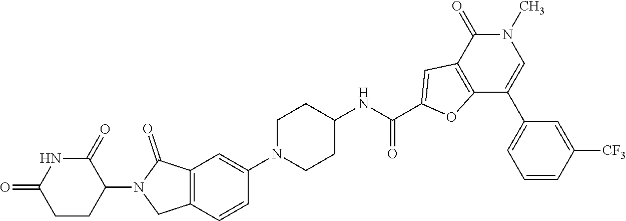 [SMILES: Cn1cc(-c2cccc(C(F)(F)F)c2)c2oc(C(=O)NC3CCN(c4ccc5c(c4)C(=O)N(C4CCC(=O)NC4=O)C5)CC3)cc2c1=O] |
| G212 | 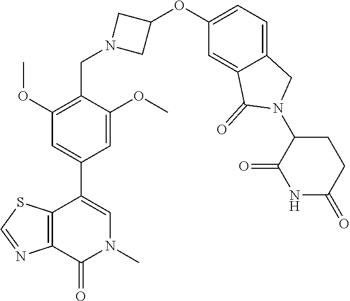 [SMILES: COc1cc(-c2cn(C)c(=O)c3ncsc23)cc(OC)c1CN1CC(Oc2ccc3c(c2)C(=O)N(C2CCC(=O)NC2=O)C3)C1] |
| G213 | 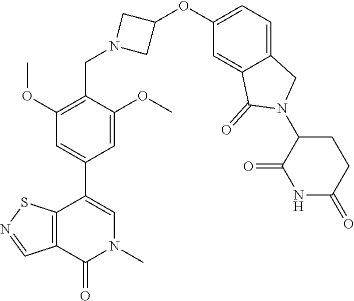 [SMILES: COc1cc(-c2cn(C)c(=O)c3cnsc23)cc(OC)c1CN1CC(Oc2ccc3c(c2)C(=O)N(C2CCC(=O)NC2=O)C3)C1] |
| G214 | 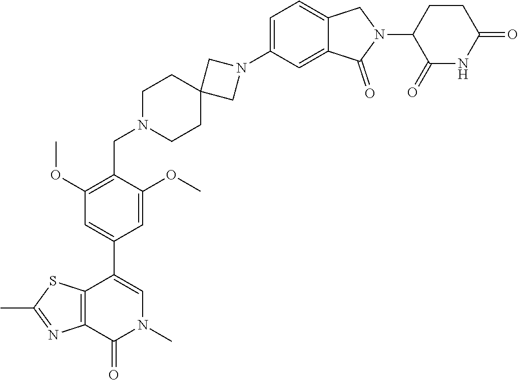 [SMILES: COc1cc(-c2cn(C)c(=O)c3nc(C)sc23)cc(OC)c1CN1CCC2(CC1)CN(c1ccc3c(c1)C(=O)N(C1CCC(=O)NC1=O)C3)C2] |
| G215 | 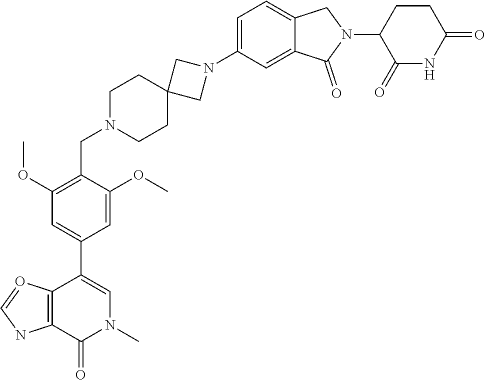 |
| G216 | 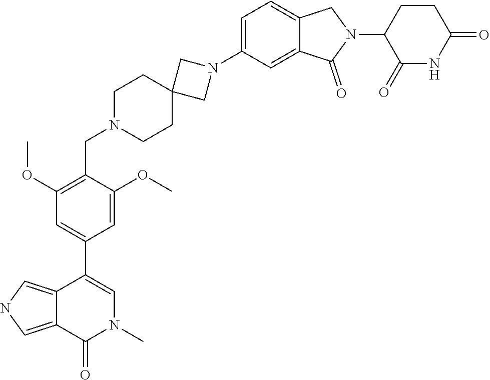 [SMILES: COc1cc(-c2cn(C)c(=O)c3c[nH]cc23)cc(OC)c1CN1CCC2(CC1)CN(c1ccc3c(c1)C(=O)N(C1CCC(=O)NC1=O)C3)C2] |
| G217 | 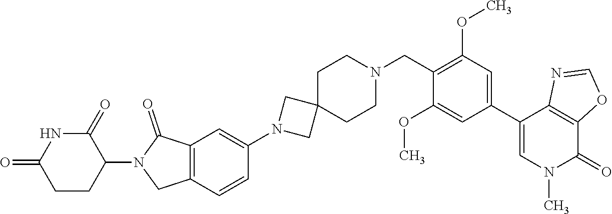 [SMILES: COc1cc(-c2cn(C)c(=O)c3ocnc23)cc(OC)c1CN1CCC2(CC1)CN(c1ccc3c(c1)C(=O)N(C1CCC(=O)NC1=O)C3)C2] |
| G218 | 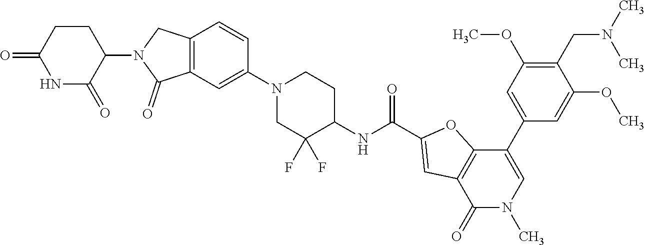 [SMILES: COc1cc(-c2cn(C)c(=O)c3cc(C(=O)NC4CCN(c5ccc6c(c5)C(=O)N(C5CCC(=O)NC5=O)C6)CC4(F)F)oc23)cc(OC)c1CN(C)C] |
| G219 | 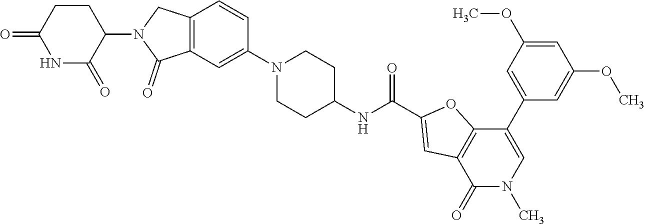 [SMILES: COc1cc(OC)cc(-c2cn(C)c(=O)c3cc(C(=O)NC4CCN(c5ccc6c(c5)C(=O)N(C5CCC(=O)NC5=O)C6)CC4)oc23)c1] |
| G220 | 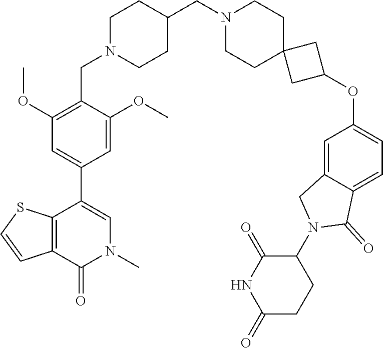 [SMILES: COc1cc(-c2cn(C)c(=O)c3ccsc23)cc(OC)c1CN1CCC(CN2CCC3(CC2)CC(Oc2ccc4c(c2)CN(C2CCC(=O)NC2=O)C4=O)C3)CC1] |
| G221 | 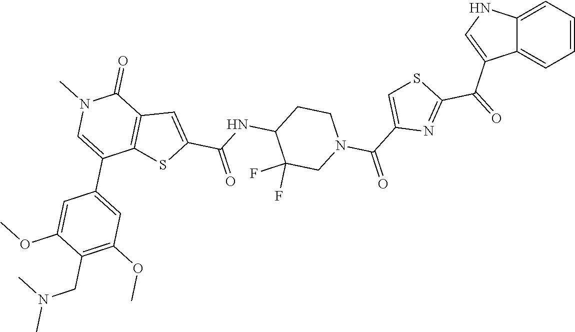 [SMILES: COc1cc(-c2cn(C)c(=O)c3cc(C(=O)NC4CCN(C(=O)c5csc(C(=O)c6c[nH]c7ccccc67)n5)CC4(F)F)sc23)cc(OC)c1CN(C)C] |
| G222 | 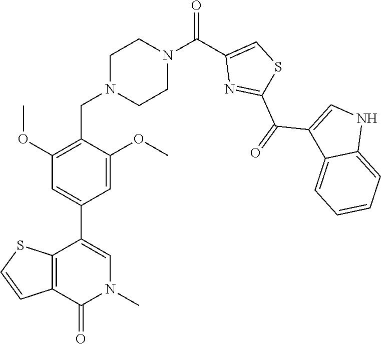 [SMILES: COc1cc(-c2cn(C)c(=O)c3ccsc23)cc(OC)c1CN1CCN(C(=O)c2csc(C(=O)c3c[nH]c4ccccc34)n2)CC1] |

or a pharmaceutically acceptable salt thereof.

The BRD9 inhibitor may be, e.g., a compound selected from the group consisting of:

| Compound | |
|---|---|
| No. | Structure |
| H1 | 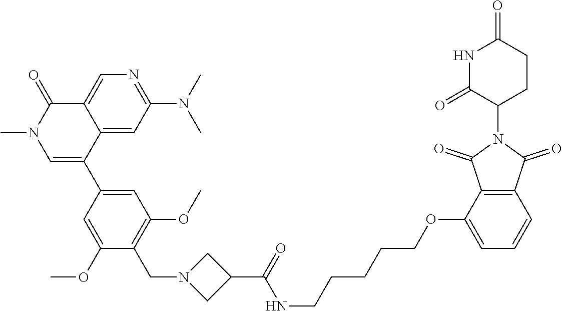 [SMILES: COc1cc(-c2cn(C)c(=O)c3cnc(N(C)C)cc23)cc(OC)c1CN1CC(C(=O)NCCCCCOc2cccc3c2C(=O)N(C2CCC(=O)NC2=O)C3=O)C1] |
| H2 | 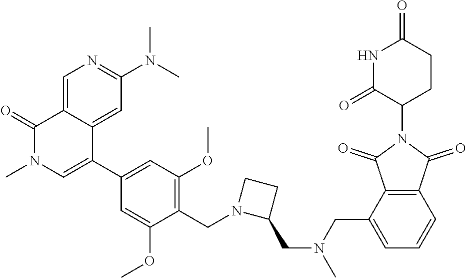 [SMILES: COc1cc(-c2cn(C)c(=O)c3cnc(N(C)C)cc23)cc(OC)c1CN1CC[C@H]1CN(C)Cc1cccc2c1C(=O)N(C1CCC(=O)NC1=O)C2=O] |
| H3 | 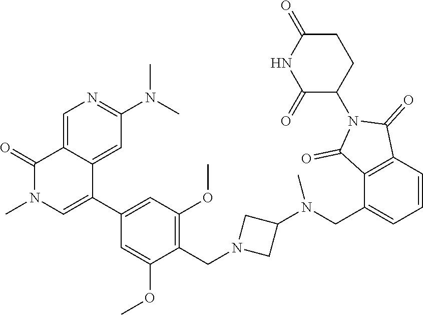 [SMILES: COc1cc(-c2cn(C)c(=O)c3cnc(N(C)C)cc23)cc(OC)c1CN1CC(N(C)Cc2cccc3c2C(=O)N(C2CCC(=O)NC2=O)C3=O)C1] |
| H4 | 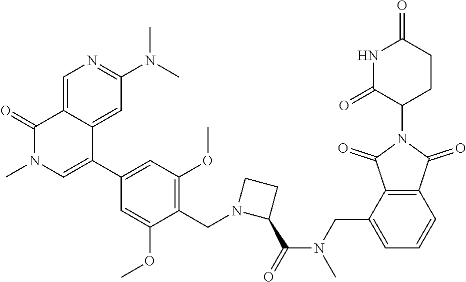 [SMILES: COc1cc(-c2cn(C)c(=O)c3cnc(N(C)C)cc23)cc(OC)c1CN1CC[C@H]1C(=O)N(C)Cc1cccc2c1C(=O)N(C1CCC(=O)NC1=O)C2=O] |
| H5 | 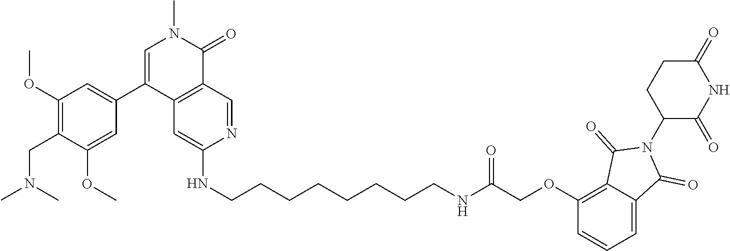 [SMILES: COc1cc(-c2cn(C)c(=O)c3cnc(NCCCCCCCCNC(=O)COc4cccc5c4C(=O)N(C4CCC(=O)NC4=O)C5=O)cc23)cc(OC)c1CN(C)C] |
| H6 | 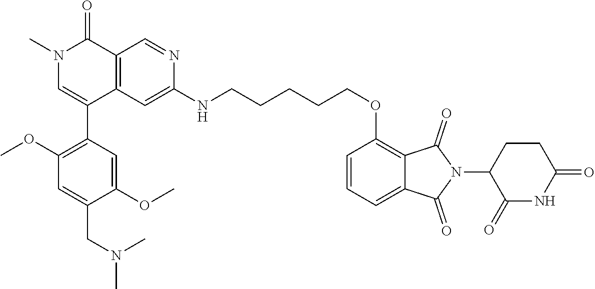 [SMILES: COc1cc(-c2cn(C)c(=O)c3cnc(NCCCCCOc4cccc5c4C(=O)N(C4CCC(=O)NC4=O)C5=O)cc23)c(OC)cc1CN(C)C] |
| H7 | 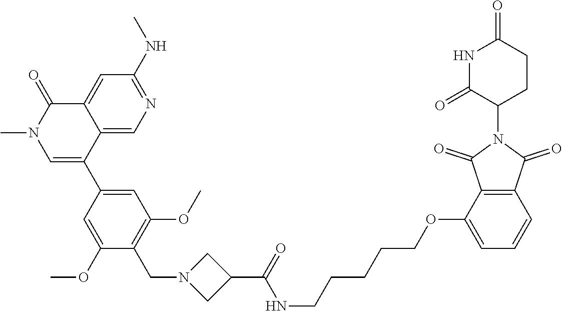 [SMILES: CNc1cc2c(=O)n(C)cc(-c3cc(OC)c(CN4CC(C(=O)NCCCCCOc5cccc6c5C(=O)N(C5CCC(=O)NC5=O)C6=O)C4)c(OC)c3)c2cn1] |
| H8 | 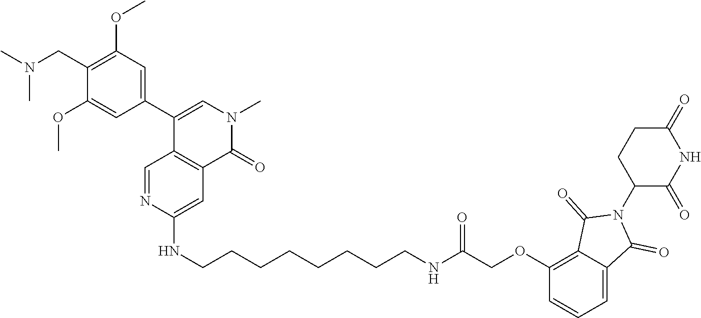 [SMILES: COc1cc(-c2cn(C)c(=O)c3cc(NCCCCCCCCNC(=O)COc4cccc5c4C(=O)N(C4CCC(=O)NC4=O)C5=O)ncc23)cc(OC)c1CN(C)C] |
| H9 | 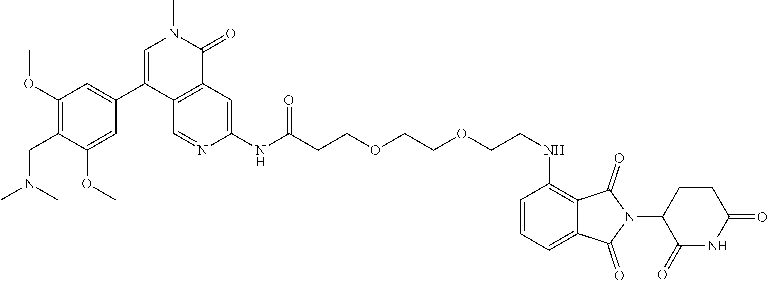 [SMILES: COc1cc(-c2cn(C)c(=O)c3cc(NC(=O)CCOCCOCCNc4cccc5c4C(=O)N(C4CCC(=O)NC4=O)C5=O)ncc23)cc(OC)c1CN(C)C] |
| H10 | 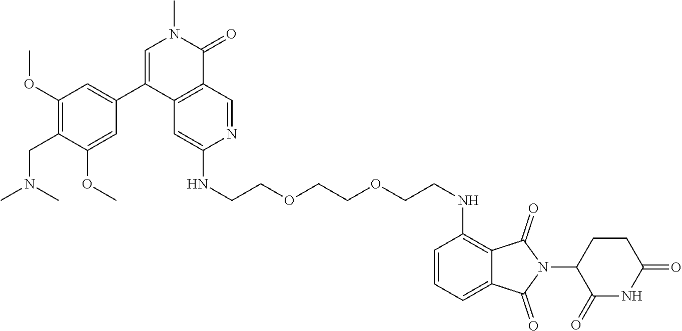 [SMILES: COc1cc(-c2cn(C)c(=O)c3cnc(NCCOCCOCCNc4cccc5c4C(=O)N(C4CCC(=O)NC4=O)C5=O)cc23)cc(OC)c1CN(C)C] |
| H11 | 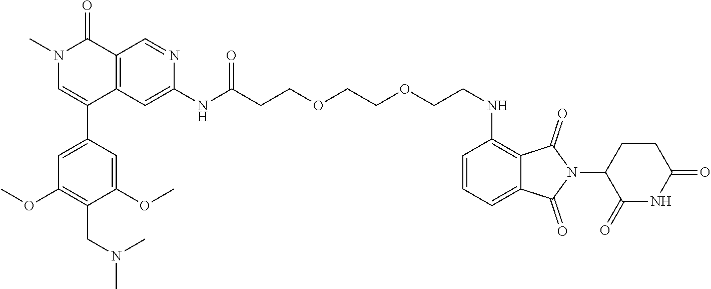 [SMILES: COc1cc(-c2cn(C)c(=O)c3cnc(NC(=O)CCOCCOCCNc4cccc5c4C(=O)N(C4CCC(=O)NC4=O)C5=O)cc23)cc(OC)c1CN(C)C] |
| H12 | 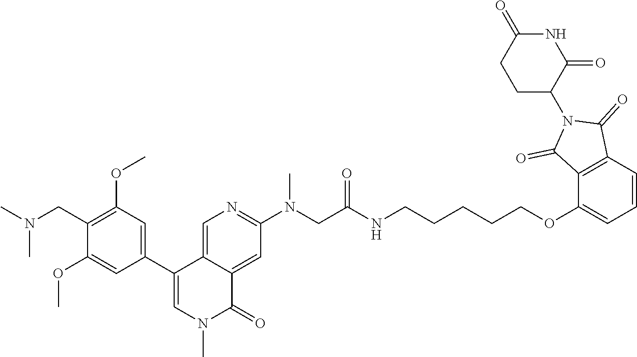 [SMILES: COc1cc(-c2cn(C)c(=O)c3cc(N(C)CC(=O)NCCCCCOc4cccc5c4C(=O)N(C4CCC(=O)NC4=O)C5=O)ncc23)cc(OC)c1CN(C)C] |
| H13 | 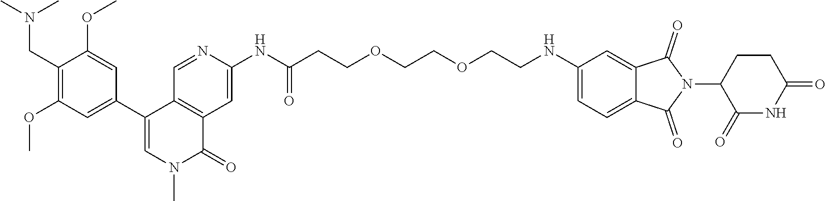 [SMILES: COc1cc(-c2cn(C)c(=O)c3cc(NC(=O)CCOCCOCCNc4ccc5c(c4)C(=O)N(C4CCC(=O)NC4=O)C5=O)ncc23)cc(OC)c1CN(C)C] |
| H14 | 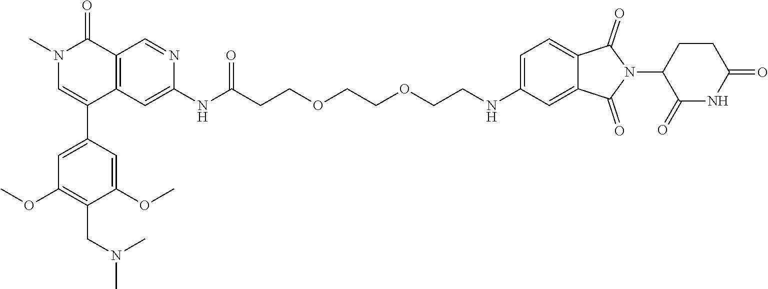 [SMILES: COc1cc(-c2cn(C)c(=O)c3cnc(NC(=O)CCOCCOCCNc4ccc5c(c4)C(=O)N(C4CCC(=O)NC4=O)C5=O)cc23)cc(OC)c1CN(C)C] |
| H15 | 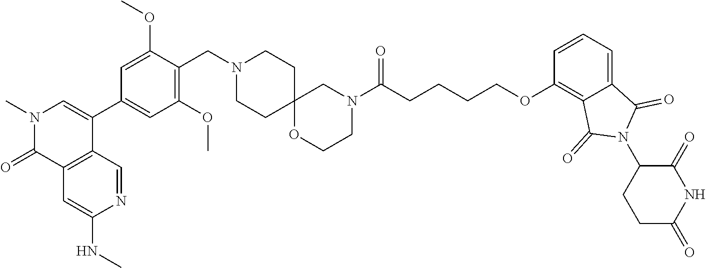 [SMILES: CNc1cc2c(=O)n(C)cc(-c3cc(OC)c(CN4CCC5(CC4)CN(C(=O)CCCCOc4cccc6c4C(=O)N(C4CCC(=O)NC4=O)C6=O)CCO5)c(OC)c3)c2cn1] |
| H16 | 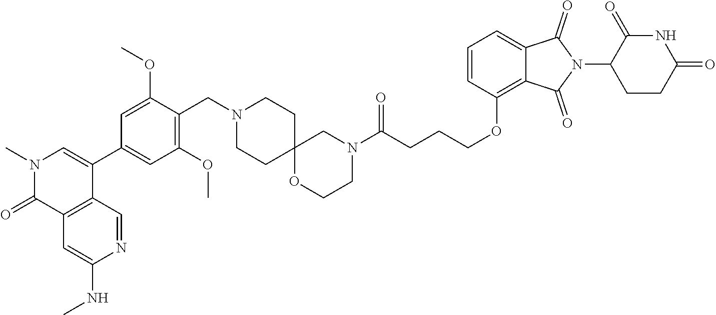 [SMILES: CNc1cc2c(=O)n(C)cc(-c3cc(OC)c(CN4CCC5(CC4)CN(C(=O)CCCOc4cccc6c4C(=O)N(C4CCC(=O)NC4=O)C6=O)CCO5)c(OC)c3)c2cn1] |
| H17 | 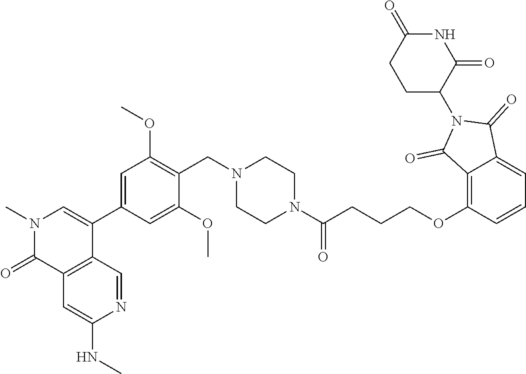 [SMILES: CNc1cc2c(=O)n(C)cc(-c3cc(OC)c(CN4CCN(C(=O)CCCOc5cccc6c5C(=O)N(C5CCC(=O)NC5=O)C6=O)CC4)c(OC)c3)c2cn1] |
| H18 | 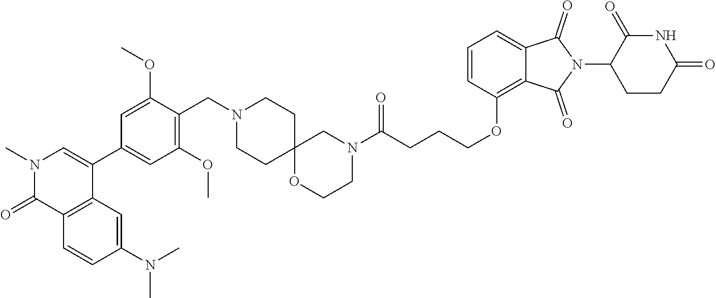 [SMILES: COc1cc(-c2cn(C)c(=O)c3ccc(N(C)C)cc23)cc(OC)c1CN1CCC2(CC1)CN(C(=O)CCCOc1cccc3c1C(=O)N(C1CCC(=O)NC1=O)C3=O)CCO2] |
| H19 | 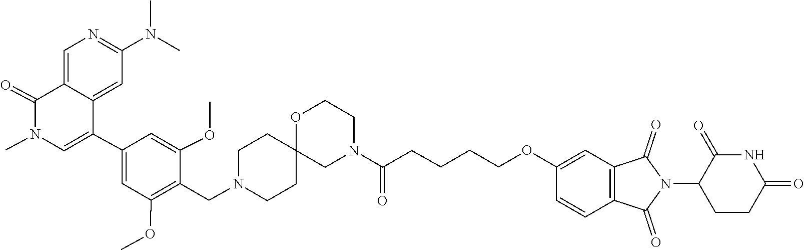 [SMILES: COc1cc(-c2cn(C)c(=O)c3cnc(N(C)C)cc23)cc(OC)c1CN1CCC2(CC1)CN(C(=O)CCCCOc1ccc3c(c1)C(=O)N(C1CCC(=O)NC1=O)C3=O)CCO2] |
| H20 | 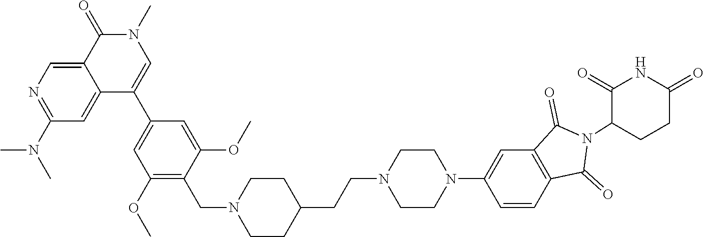 [SMILES: COc1cc(-c2cn(C)c(=O)c3cnc(N(C)C)cc23)cc(OC)c1CN1CCC(CCN2CCN(c3ccc4c(c3)C(=O)N(C3CCC(=O)NC3=O)C4=O)CC2)CC1] |
| H21 | 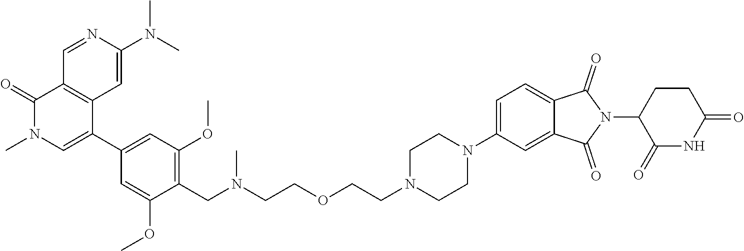 [SMILES: COc1cc(-c2cn(C)c(=O)c3cnc(N(C)C)cc23)cc(OC)c1CN(C)CCOCCN1CCN(c2ccc3c(c2)C(=O)N(C2CCC(=O)NC2=O)C3=O)CC1] |
| H22 | 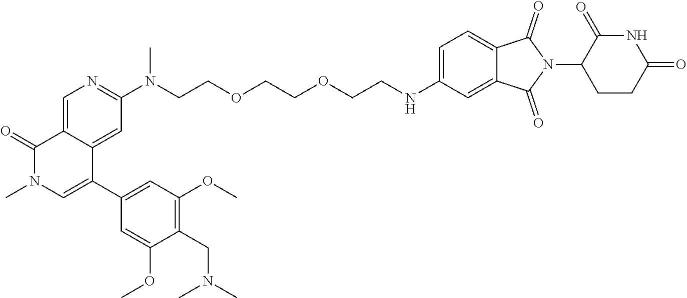 [SMILES: COc1cc(-c2cn(C)c(=O)c3cnc(N(C)CCOCCOCCNc4ccc5c(c4)C(=O)N(C4CCC(=O)NC4=O)C5=O)cc23)cc(OC)c1CN(C)C] |
| H23 | 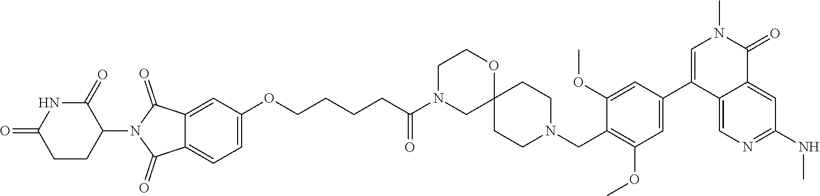 [SMILES: CNc1cc2c(=O)n(C)cc(-c3cc(OC)c(CN4CCC5(CC4)CN(C(=O)CCCCOc4ccc6c(c4)C(=O)N(C4CCC(=O)NC4=O)C6=O)CCO5)c(OC)c3)c2cn1] |
| H24 | 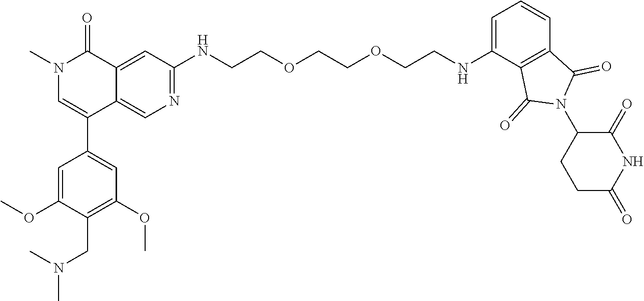 [SMILES: COc1cc(-c2cn(C)c(=O)c3cc(NCCOCCOCCNc4cccc5c4C(=O)N(C4CCC(=O)NC4=O)C5=O)ncc23)cc(OC)c1CN(C)C] |
| H25 | 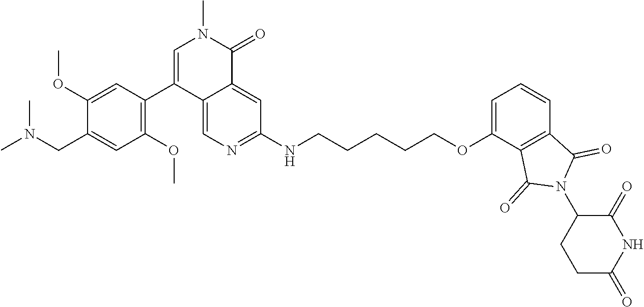 [SMILES: COc1cc(-c2cn(C)c(=O)c3cc(NCCCCCOc4cccc5c4C(=O)N(C4CCC(=O)NC4=O)C5=O)ncc23)c(OC)cc1CN(C)C] |
| H26 | 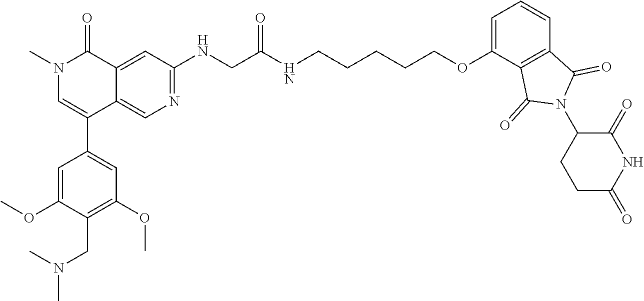 [SMILES: COc1cc(-c2cn(C)c(=O)c3cc(NCC(=O)NCCCCCOc4cccc5c4C(=O)N(C4CCC(=O)NC4=O)C5=O)ncc23)cc(OC)c1CN(C)C] |
| H27 | 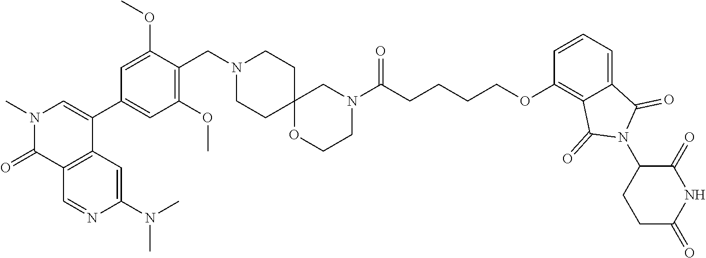 [SMILES: COc1cc(-c2cn(C)c(=O)c3cnc(N(C)C)cc23)cc(OC)c1CN1CCC2(CC1)CN(C(=O)CCCCOc1cccc3c1C(=O)N(C1CCC(=O)NC1=O)C3=O)CCO2] |
| H28 | 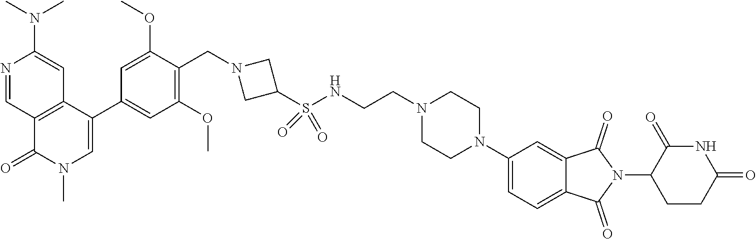 [SMILES: COc1cc(-c2cn(C)c(=O)c3cnc(N(C)C)cc23)cc(OC)c1CN1CC(S(=O)(=O)NCCN2CCN(c3ccc4c(c3)C(=O)N(C3CCC(=O)NC3=O)C4=O)CC2)C1] |
| H29 | 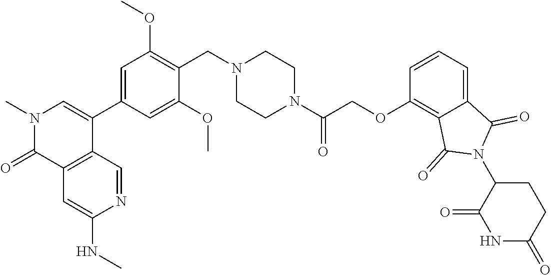 [SMILES: CNc1cc2c(=O)n(C)cc(-c3cc(OC)c(CN4CCN(C(=O)COc5cccc6c5C(=O)N(C5CCC(=O)NC5=O)C6=O)CC4)c(OC)c3)c2cn1] |
| H30 | 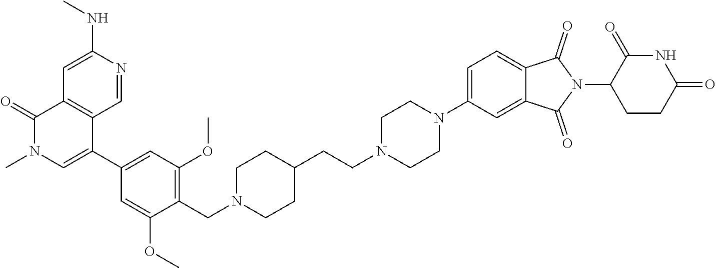 [SMILES: CNc1cc2c(=O)n(C)cc(-c3cc(OC)c(CN4CCC(CCN5CCN(c6ccc7c(c6)C(=O)N(C6CCC(=O)NC6=O)C7=O)CC5)CC4)c(OC)c3)c2cn1] |
| H31 | 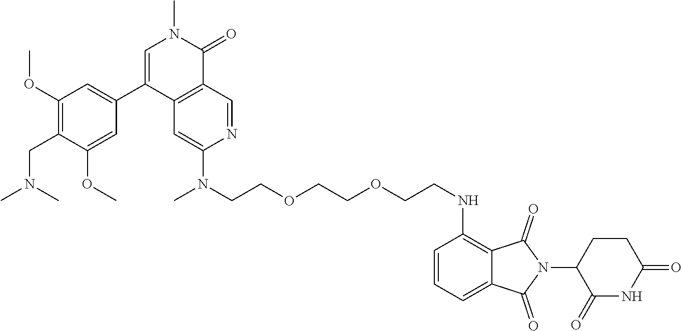 [SMILES: COc1cc(-c2cn(C)c(=O)c3cnc(N(C)CCOCCOCCNc4cccc5c4C(=O)N(C4CCC(=O)NC4=O)C5=O)cc23)cc(OC)c1CN(C)C] |
| H32 | 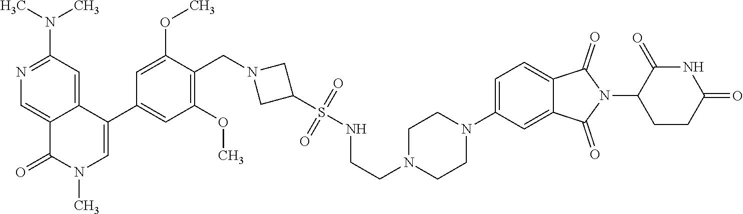 [SMILES: COc1cc(-c2cn(C)c(=O)c3cnc(N(C)C)cc23)cc(OC)c1CN1CC(S(=O)(=O)NCCN2CCN(c3ccc4c(c3)C(=O)N(C3CCC(=O)NC3=O)C4=O)CC2)C1] |
| H33 | 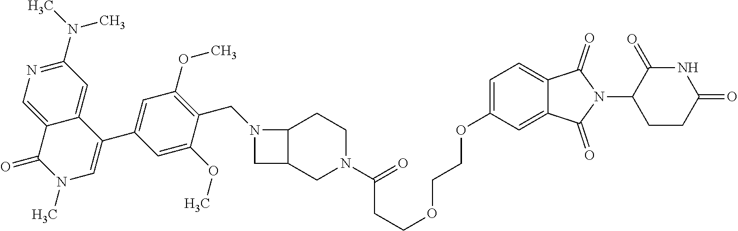 [SMILES: COc1cc(-c2cn(C)c(=O)c3cnc(N(C)C)cc23)cc(OC)c1CN1CC2CN(C(=O)CCOCCOc3ccc4c(c3)C(=O)N(C3CCC(=O)NC3=O)C4=O)CCC21] |
| H34 | 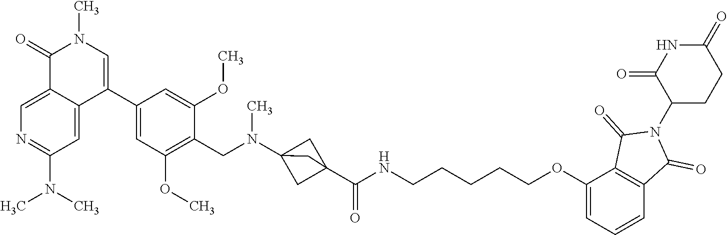 [SMILES: COc1cc(-c2cn(C)c(=O)c3cnc(N(C)C)cc23)cc(OC)c1CN(C)C12CC(C(=O)NCCCCCOc3cccc4c3C(=O)N(C3CCC(=O)NC3=O)C4=O)(C1)C2] |
| H35 | 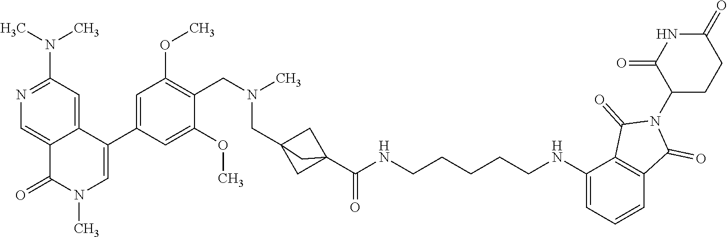 [SMILES: COc1cc(-c2cn(C)c(=O)c3cnc(N(C)C)cc23)cc(OC)c1CN(C)CC12CC(C(=O)NCCCCCNc3cccc4c3C(=O)N(C3CCC(=O)NC3=O)C4=O)(C1)C2] |
| H36 | 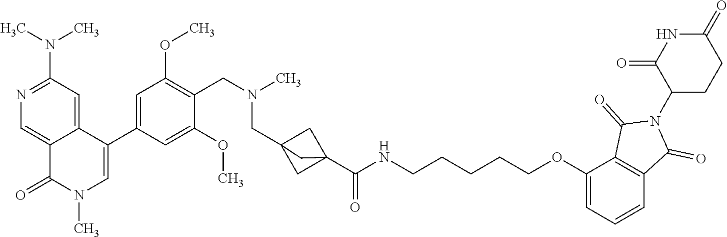 [SMILES: COc1cc(-c2cn(C)c(=O)c3cnc(N(C)C)cc23)cc(OC)c1CN(C)CC12CC(C(=O)NCCCCCOc3cccc4c3C(=O)N(C3CCC(=O)NC3=O)C4=O)(C1)C2] |
| H37 | 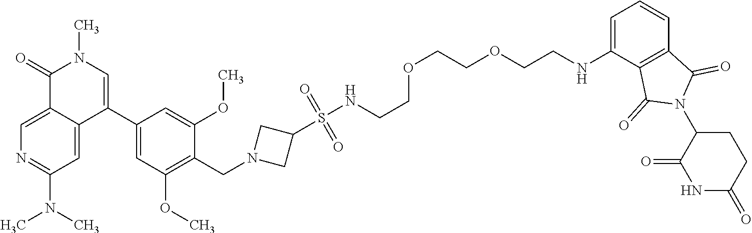 [SMILES: COc1cc(-c2cn(C)c(=O)c3cnc(N(C)C)cc23)cc(OC)c1CN1CC(S(=O)(=O)NCCOCCOCCNc2cccc3c2C(=O)N(C2CCC(=O)NC2=O)C3=O)C1] |
| H38 | 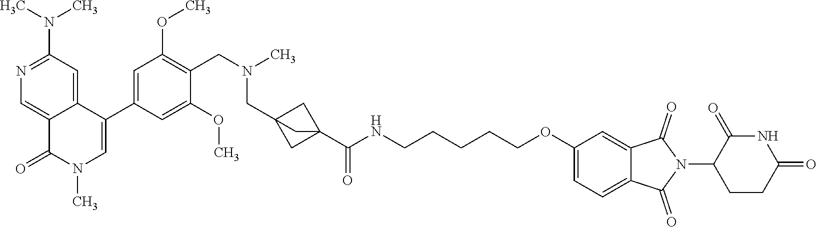 [SMILES: COc1cc(-c2cn(C)c(=O)c3cnc(N(C)C)cc23)cc(OC)c1CN(C)CC12CC(C(=O)NCCCCCOc3ccc4c(c3)C(=O)N(C3CCC(=O)NC3=O)C4=O)(C1)C2] |
| H39 | 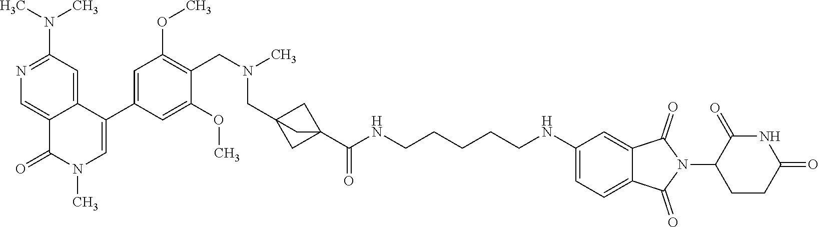 [SMILES: COc1cc(-c2cn(C)c(=O)c3cnc(N(C)C)cc23)cc(OC)c1CN(C)CC12CC(C(=O)NCCCCCNc3ccc4c(c3)C(=O)N(C3CCC(=O)NC3=O)C4=O)(C1)C2] |
| H40 | 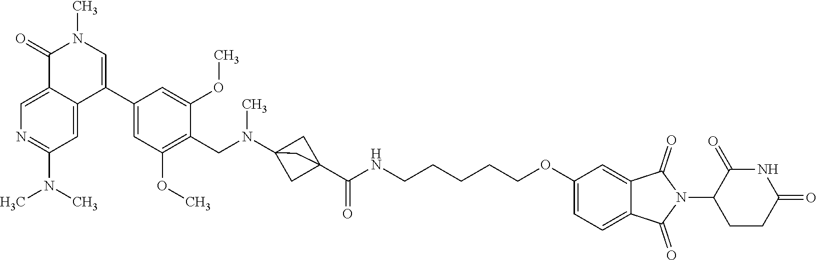 [SMILES: COc1cc(-c2cn(C)c(=O)c3cnc(N(C)C)cc23)cc(OC)c1CN(C)C12CC(C(=O)NCCCCCOc3ccc4c(c3)C(=O)N(C3CCC(=O)NC3=O)C4=O)(C1)C2] |
| H41 | 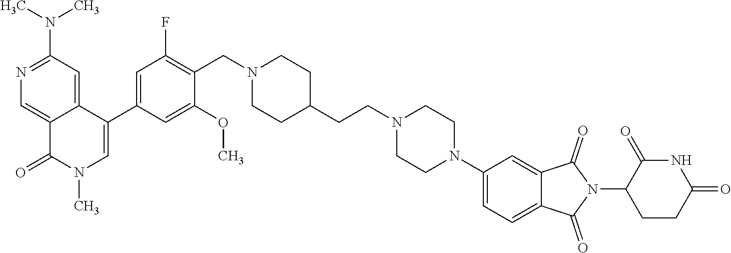 [SMILES: COc1cc(-c2cn(C)c(=O)c3cnc(N(C)C)cc23)cc(F)c1CN1CCC(CCN2CCN(c3ccc4c(c3)C(=O)N(C3CCC(=O)NC3=O)C4=O)CC2)CC1] |
| H42 | 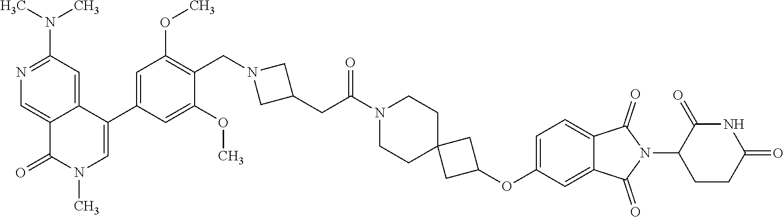 [SMILES: COc1cc(-c2cn(C)c(=O)c3cnc(N(C)C)cc23)cc(OC)c1CN1CC(CC(=O)N2CCC3(CC2)CC(Oc2ccc4c(c2)C(=O)N(C2CCC(=O)NC2=O)C4=O)C3)C1] |
| H43 | 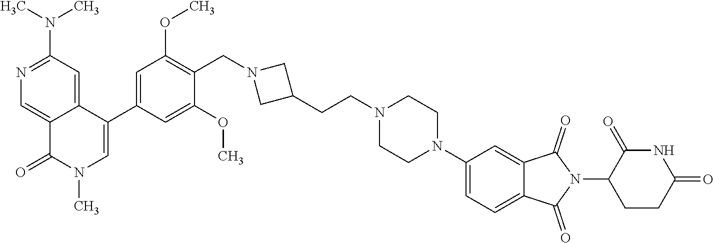 [SMILES: COc1cc(-c2cn(C)c(=O)c3cnc(N(C)C)cc23)cc(OC)c1CN1CC(CCN2CCN(c3ccc4c(c3)C(=O)N(C3CCC(=O)NC3=O)C4=O)CC2)C1] |
| H44 | 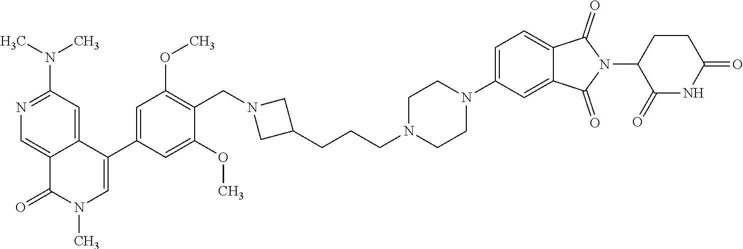 [SMILES: COc1cc(-c2cn(C)c(=O)c3cnc(N(C)C)cc23)cc(OC)c1CN1CC(CCCN2CCN(c3ccc4c(c3)C(=O)N(C3CCC(=O)NC3=O)C4=O)CC2)C1] |
| H45 | 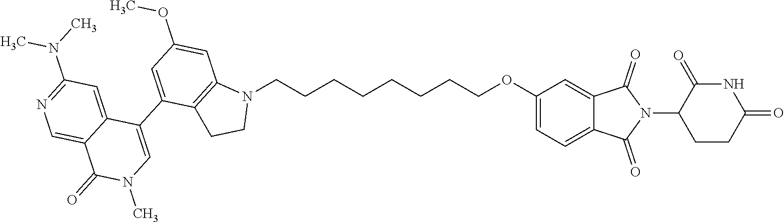 [SMILES: COc1cc(-c2cn(C)c(=O)c3cnc(N(C)C)cc23)c2c(c1)N(CCCCCCCCOc1ccc3c(c1)C(=O)N(C1CCC(=O)NC1=O)C3=O)CC2] |
| H46 | 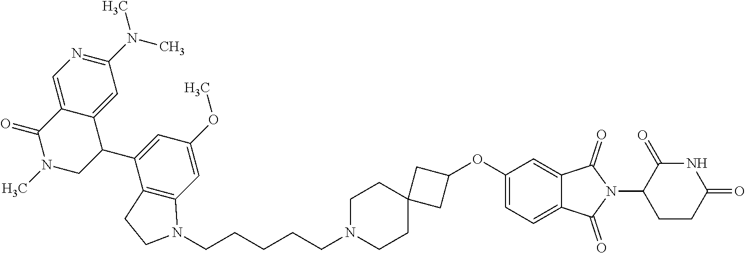 [SMILES: COc1cc(C2CN(C)C(=O)c3cnc(N(C)C)cc32)c2c(c1)N(CCCCCN1CCC3(CC1)CC(Oc1ccc4c(c1)C(=O)N(C1CCC(=O)NC1=O)C4=O)C3)CC2] |
| H47 | 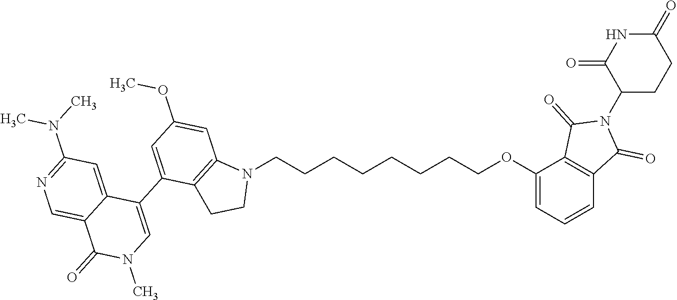 [SMILES: COc1cc(-c2cn(C)c(=O)c3cnc(N(C)C)cc23)c2c(c1)N(CCCCCCCCOc1cccc3c1C(=O)N(C1CCC(=O)NC1=O)C3=O)CC2] |
| H48 | 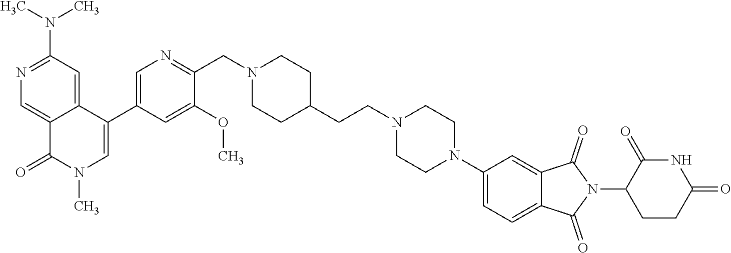 [SMILES: COc1cc(-c2cn(C)c(=O)c3cnc(N(C)C)cc23)cnc1CN1CCC(CCN2CCN(c3ccc4c(c3)C(=O)N(C3CCC(=O)NC3=O)C4=O)CC2)CC1] |
| H49 | 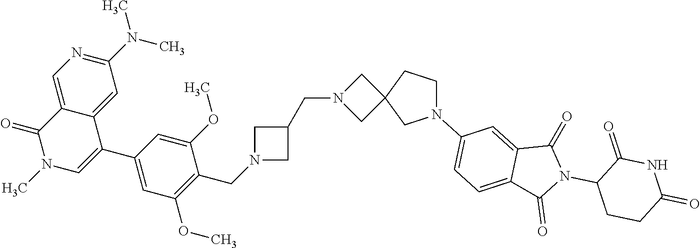 [SMILES: COc1cc(-c2cn(C)c(=O)c3cnc(N(C)C)cc23)cc(OC)c1CN1CC(CN2CC3(CCN(c4ccc5c(c4)C(=O)N(C4CCC(=O)NC4=O)C5=O)C3)C2)C1] |
| H50 | 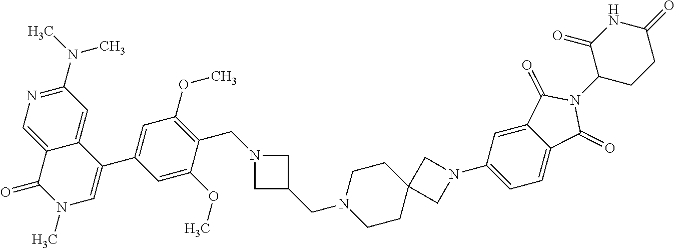 [SMILES: COc1cc(-c2cn(C)c(=O)c3cnc(N(C)C)cc23)cc(OC)c1CN1CC(CN2CCC3(CC2)CN(c2ccc4c(c2)C(=O)N(C2CCC(=O)NC2=O)C4=O)C3)C1] |
| H51 | 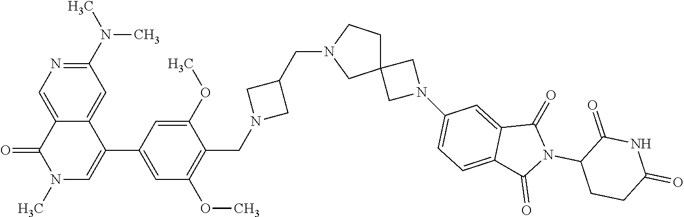 [SMILES: COc1cc(-c2cn(C)c(=O)c3cnc(N(C)C)cc23)cc(OC)c1CN1CC(CN2CCC3(C2)CN(c2ccc4c(c2)C(=O)N(C2CCC(=O)NC2=O)C4=O)C3)C1] |
| H52 | 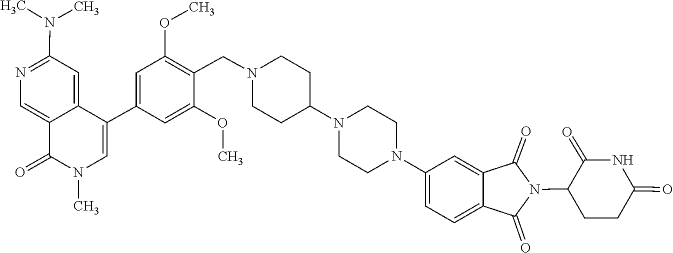 [SMILES: COc1cc(-c2cn(C)c(=O)c3cnc(N(C)C)cc23)cc(OC)c1CN1CCC(N2CCN(c3ccc4c(c3)C(=O)N(C3CCC(=O)NC3=O)C4=O)CC2)CC1] |
| H53 | 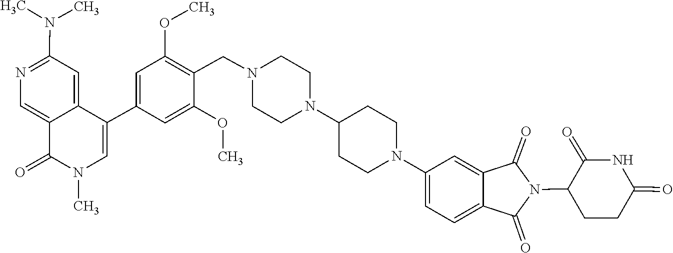 [SMILES: COc1cc(-c2cn(C)c(=O)c3cnc(N(C)C)cc23)cc(OC)c1CN1CCN(C2CCN(c3ccc4c(c3)C(=O)N(C3CCC(=O)NC3=O)C4=O)CC2)CC1] |
| H54 | 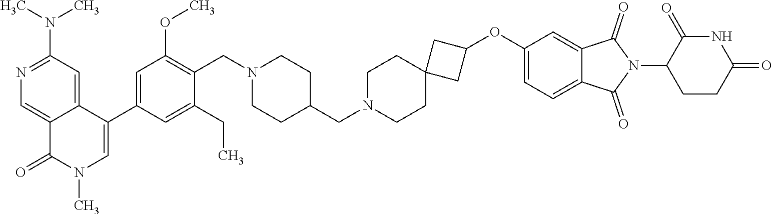 [SMILES: CCc1cc(-c2cn(C)c(=O)c3cnc(N(C)C)cc23)cc(OC)c1CN1CCC(CN2CCC3(CC2)CC(Oc2ccc4c(c2)C(=O)N(C2CCC(=O)NC2=O)C4=O)C3)CC1] |
| H55 | 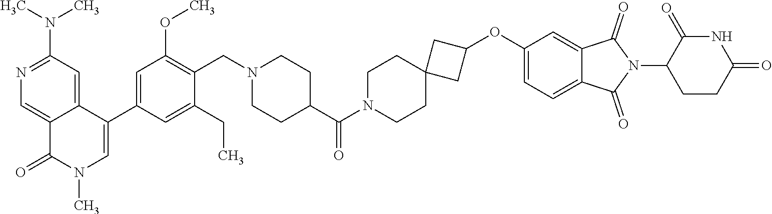 [SMILES: CCc1cc(-c2cn(C)c(=O)c3cnc(N(C)C)cc23)cc(OC)c1CN1CCC(C(=O)N2CCC3(CC2)CC(Oc2ccc4c(c2)C(=O)N(C2CCC(=O)NC2=O)C4=O)C3)CC1] |
| H56 | 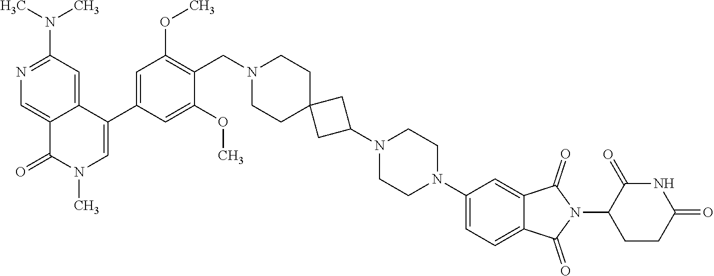 [SMILES: COc1cc(-c2cn(C)c(=O)c3cnc(N(C)C)cc23)cc(OC)c1CN1CCC2(CC1)CC(N1CCN(c3ccc4c(c3)C(=O)N(C3CCC(=O)NC3=O)C4=O)CC1)C2] |
| H57 | 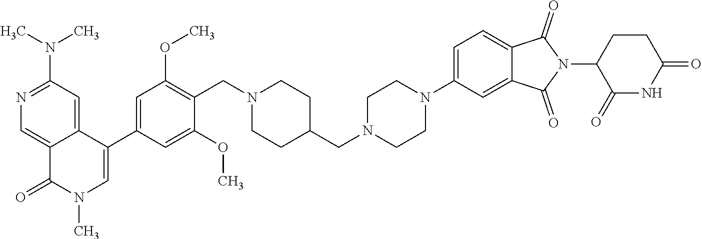 [SMILES: COc1cc(-c2cn(C)c(=O)c3cnc(N(C)C)cc23)cc(OC)c1CN1CCC(CN2CCN(c3ccc4c(c3)C(=O)N(C3CCC(=O)NC3=O)C4=O)CC2)CC1] |
| H58 | 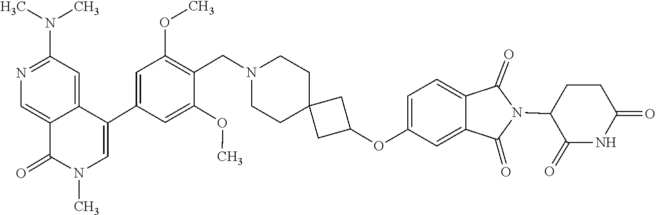 [SMILES: COc1cc(-c2cn(C)c(=O)c3cnc(N(C)C)cc23)cc(OC)c1CN1CCC2(CC1)CC(Oc1ccc3c(c1)C(=O)N(C1CCC(=O)NC1=O)C3=O)C2] |
| H59 | 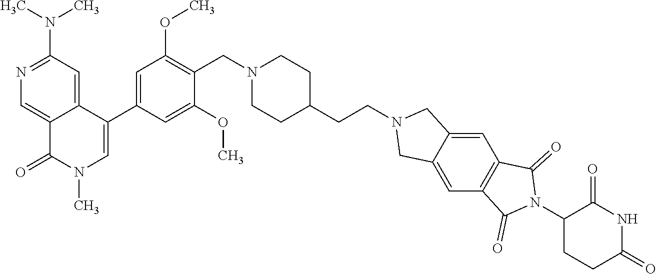 [SMILES: COc1cc(-c2cn(C)c(=O)c3cnc(N(C)C)cc23)cc(OC)c1CN1CCC(CCN2Cc3cc4c(cc3C2)C(=O)N(C2CCC(=O)NC2=O)C4=O)CC1] |
| H60 | 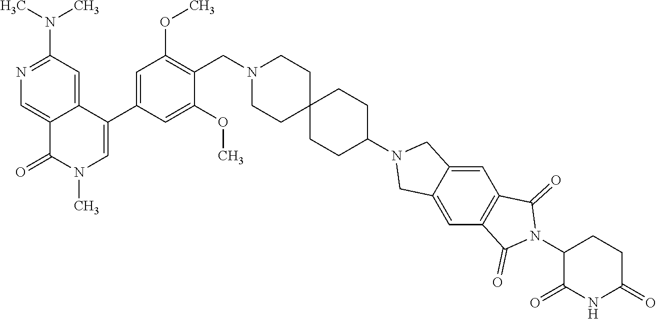 [SMILES: COc1cc(-c2cn(C)c(=O)c3cnc(N(C)C)cc23)cc(OC)c1CN1CCC2(CCC(N3Cc4cc5c(cc4C3)C(=O)N(C3CCC(=O)NC3=O)C5=O)CC2)CC1] |
| H61 | 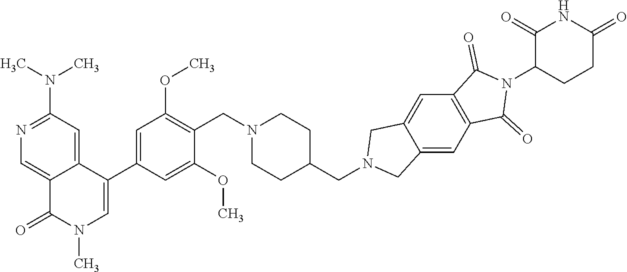 [SMILES: COc1cc(-c2cn(C)c(=O)c3cnc(N(C)C)cc23)cc(OC)c1CN1CCC(CN2Cc3cc4c(cc3C2)C(=O)N(C2CCC(=O)NC2=O)C4=O)CC1] |
| H62 | 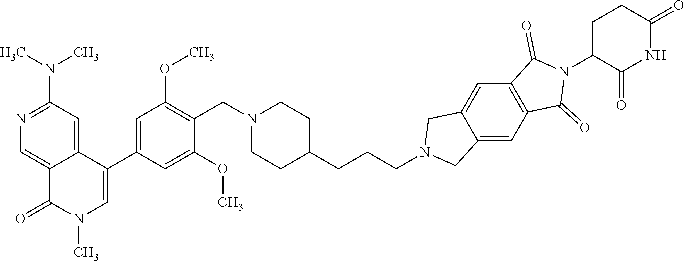 [SMILES: COc1cc(-c2cn(C)c(=O)c3cnc(N(C)C)cc23)cc(OC)c1CN1CCC(CCCN2Cc3cc4c(cc3C2)C(=O)N(C2CCC(=O)NC2=O)C4=O)CC1] |
| H63 | 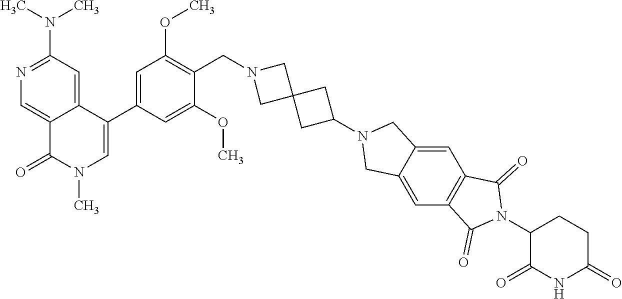 [SMILES: COc1cc(-c2cn(C)c(=O)c3cnc(N(C)C)cc23)cc(OC)c1CN1CC2(CC(N3Cc4cc5c(cc4C3)C(=O)N(C3CCC(=O)NC3=O)C5=O)C2)C1] |
| H64 | 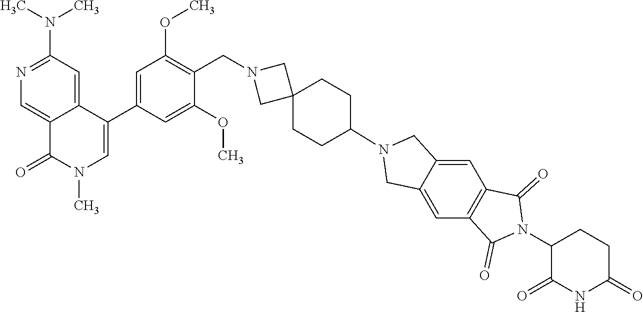 [SMILES: COc1cc(-c2cn(C)c(=O)c3cnc(N(C)C)cc23)cc(OC)c1CN1CC2(CCC(N3Cc4cc5c(cc4C3)C(=O)N(C3CCC(=O)NC3=O)C5=O)CC2)C1] |
| H65 | 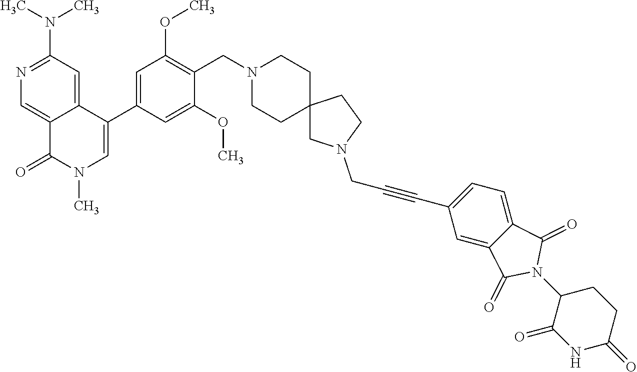 [SMILES: COc1cc(-c2cn(C)c(=O)c3cnc(N(C)C)cc23)cc(OC)c1CN1CCC2(CC1)CCN(CC#Cc1ccc3c(c1)C(=O)N(C1CCC(=O)NC1=O)C3=O)C2] |
| H66 | 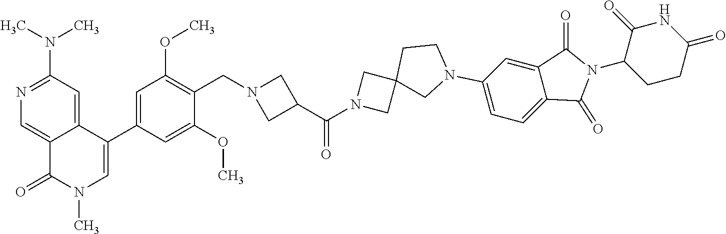 [SMILES: COc1cc(-c2cn(C)c(=O)c3cnc(N(C)C)cc23)cc(OC)c1CN1CC(C(=O)N2CC3(CCN(c4ccc5c(c4)C(=O)N(C4CCC(=O)NC4=O)C5=O)C3)C2)C1] |
| H67 | 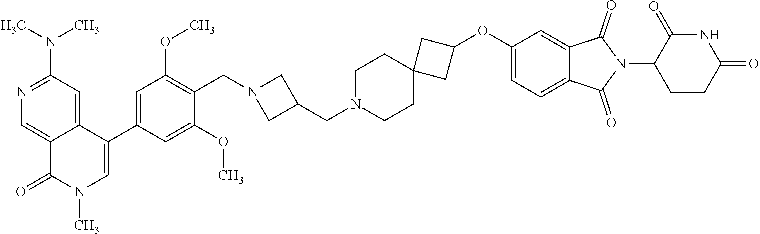 [SMILES: COc1cc(-c2cn(C)c(=O)c3cnc(N(C)C)cc23)cc(OC)c1CN1CC(CN2CCC3(CC2)CC(Oc2ccc4c(c2)C(=O)N(C2CCC(=O)NC2=O)C4=O)C3)C1] |
| H68 | 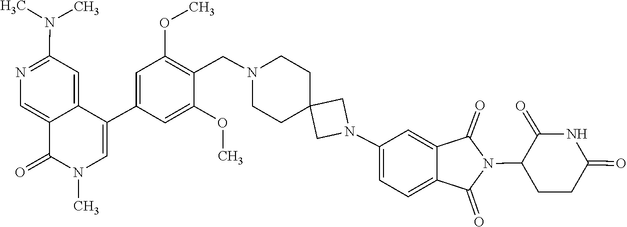 [SMILES: COc1cc(-c2cn(C)c(=O)c3cnc(N(C)C)cc23)cc(OC)c1CN1CCC2(CC1)CN(c1ccc3c(c1)C(=O)N(C1CCC(=O)NC1=O)C3=O)C2] |
| H69 | 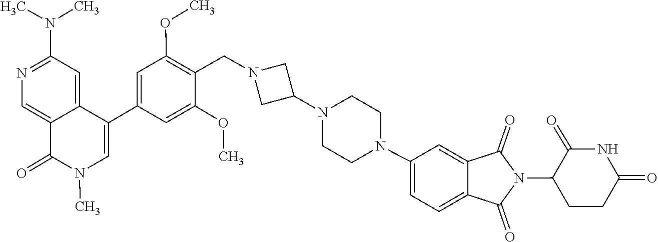 [SMILES: COc1cc(-c2cn(C)c(=O)c3cnc(N(C)C)cc23)cc(OC)c1CN1CC(N2CCN(c3ccc4c(c3)C(=O)N(C3CCC(=O)NC3=O)C4=O)CC2)C1] |
| H70 | 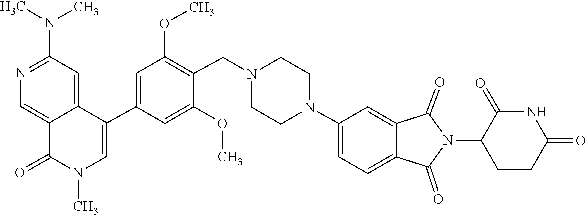 [SMILES: COc1cc(-c2cn(C)c(=O)c3cnc(N(C)C)cc23)cc(OC)c1CN1CCN(c2ccc3c(c2)C(=O)N(C2CCC(=O)NC2=O)C3=O)CC1] |
| H71 | 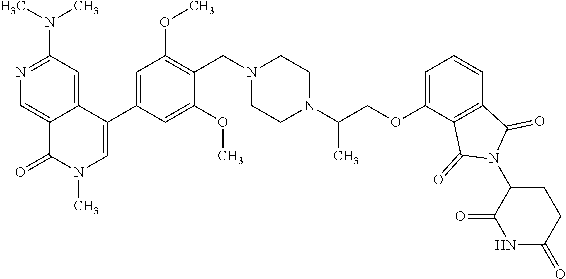 [SMILES: COc1cc(-c2cn(C)c(=O)c3cnc(N(C)C)cc23)cc(OC)c1CN1CCN(C(C)COc2cccc3c2C(=O)N(C2CCC(=O)NC2=O)C3=O)CC1] |
| H72 | 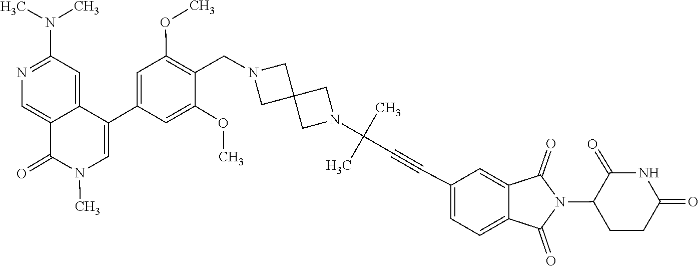 [SMILES: COc1cc(-c2cn(C)c(=O)c3cnc(N(C)C)cc23)cc(OC)c1CN1CC2(C1)CN(C(C)(C)C#Cc1ccc3c(c1)C(=O)N(C1CCC(=O)NC1=O)C3=O)C2] |
| H73 | 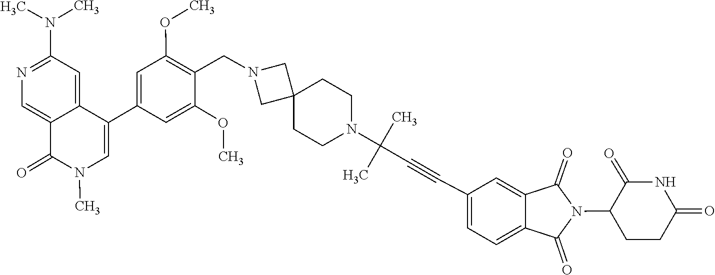 [SMILES: COc1cc(-c2cn(C)c(=O)c3cnc(N(C)C)cc23)cc(OC)c1CN1CC2(CCN(C(C)(C)C#Cc3ccc4c(c3)C(=O)N(C3CCC(=O)NC3=O)C4=O)CC2)C1] |
| H74 | 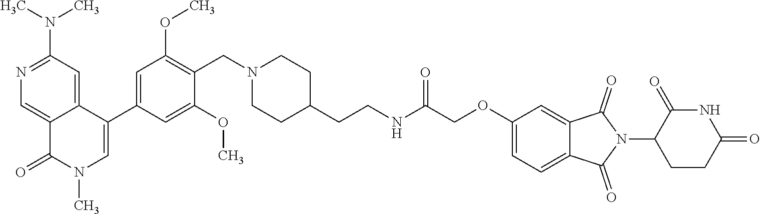 [SMILES: COc1cc(-c2cn(C)c(=O)c3cnc(N(C)C)cc23)cc(OC)c1CN1CCC(CCNC(=O)COc2ccc3c(c2)C(=O)N(C2CCC(=O)NC2=O)C3=O)CC1] |
| H75 | 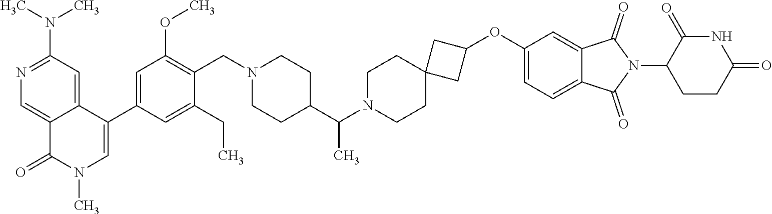 [SMILES: CCc1cc(-c2cn(C)c(=O)c3cnc(N(C)C)cc23)cc(OC)c1CN1CCC(C(C)N2CCC3(CC2)CC(Oc2ccc4c(c2)C(=O)N(C2CCC(=O)NC2=O)C4=O)C3)CC1] |
| H76 | 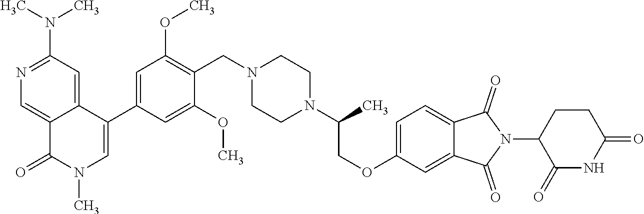 [SMILES: COc1cc(-c2cn(C)c(=O)c3cnc(N(C)C)cc23)cc(OC)c1CN1CCN([C@@H](C)COc2ccc3c(c2)C(=O)N(C2CCC(=O)NC2=O)C3=O)CC1] |
| H77 | 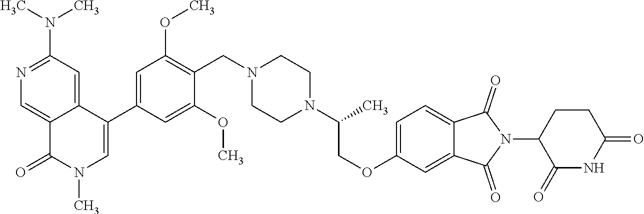 [SMILES: COc1cc(-c2cn(C)c(=O)c3cnc(N(C)C)cc23)cc(OC)c1CN1CCN([C@H](C)COc2ccc3c(c2)C(=O)N(C2CCC(=O)NC2=O)C3=O)CC1] |
| H78 | 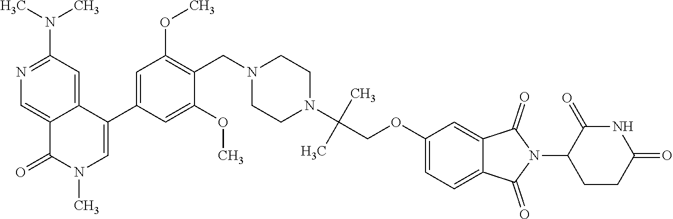 [SMILES: COc1cc(-c2cn(C)c(=O)c3cnc(N(C)C)cc23)cc(OC)c1CN1CCN(C(C)(C)COc2ccc3c(c2)C(=O)N(C2CCC(=O)NC2=O)C3=O)CC1] |
| H79 | 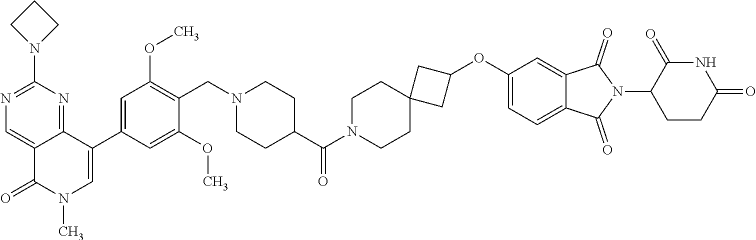 [SMILES: COc1cc(-c2cn(C)c(=O)c3cnc(N4CCC4)nc23)cc(OC)c1CN1CCC(C(=O)N2CCC3(CC2)CC(Oc2ccc4c(c2)C(=O)N(C2CCC(=O)NC2=O)C4=O)C3)CC1] |
| H80 | 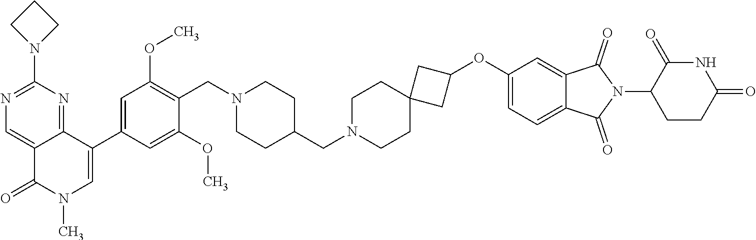 [SMILES: COc1cc(-c2cn(C)c(=O)c3cnc(N4CCC4)nc23)cc(OC)c1CN1CCC(CN2CCC3(CC2)CC(Oc2ccc4c(c2)C(=O)N(C2CCC(=O)NC2=O)C4=O)C3)CC1] |
| H81 | 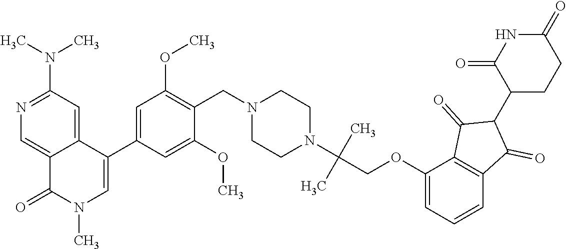 [SMILES: COc1cc(-c2cn(C)c(=O)c3cnc(N(C)C)cc23)cc(OC)c1CN1CCN(C(C)(C)COc2cccc3c2C(=O)C(C2CCC(=O)NC2=O)C3=O)CC1] |
| H82 | 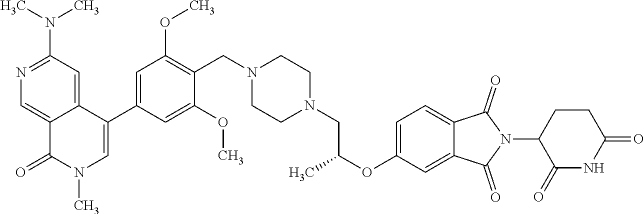 [SMILES: COc1cc(-c2cn(C)c(=O)c3cnc(N(C)C)cc23)cc(OC)c1CN1CCN(C[C@@H](C)Oc2ccc3c(c2)C(=O)N(C2CCC(=O)NC2=O)C3=O)CC1] |
| H83 | 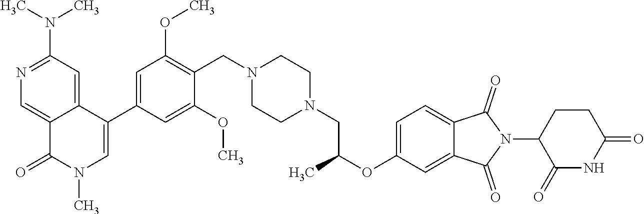 [SMILES: COc1cc(-c2cn(C)c(=O)c3cnc(N(C)C)cc23)cc(OC)c1CN1CCN(C[C@H](C)Oc2ccc3c(c2)C(=O)N(C2CCC(=O)NC2=O)C3=O)CC1] |
| H84 | 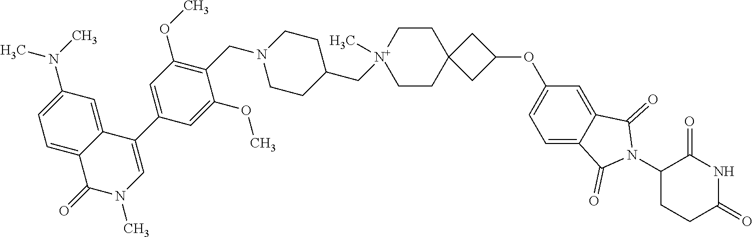 [SMILES: COc1cc(-c2cn(C)c(=O)c3ccc(N(C)C)cc23)cc(OC)c1CN1CCC(C[N+]2(C)CCC3(CC2)CC(Oc2ccc4c(c2)C(=O)N(C2CCC(=O)NC2=O)C4=O)C3)CC1] |
| H85 | 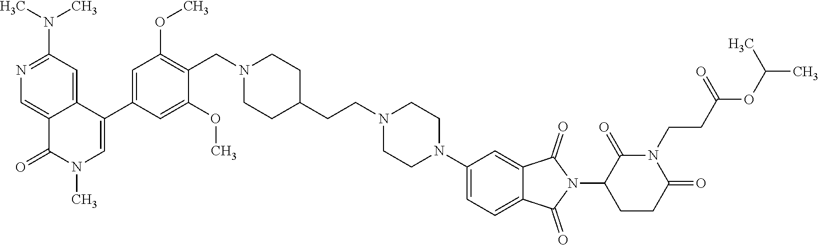 [SMILES: COc1cc(-c2cn(C)c(=O)c3cnc(N(C)C)cc23)cc(OC)c1CN1CCC(CCN2CCN(c3ccc4c(c3)C(=O)N(C3CCC(=O)N(CCC(=O)OC(C)C)C3=O)C4=O)CC2)CC1] |
| H86 | 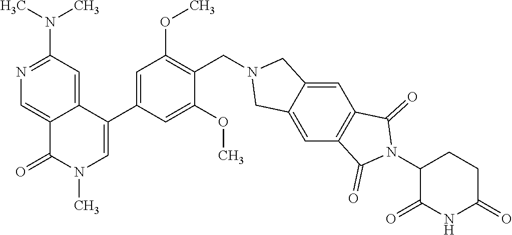 [SMILES: COc1cc(-c2cn(C)c(=O)c3cnc(N(C)C)cc23)cc(OC)c1CN1Cc2cc3c(cc2C1)C(=O)N(C1CCC(=O)NC1=O)C3=O] |
| H87 | 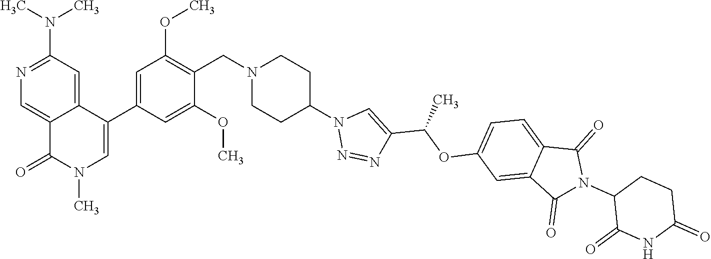 [SMILES: COc1cc(-c2cn(C)c(=O)c3cnc(N(C)C)cc23)cc(OC)c1CN1CCC(n2cc([C@H](C)Oc3ccc4c(c3)C(=O)N(C3CCC(=O)NC3=O)C4=O)nn2)CC1] |
| H88 | 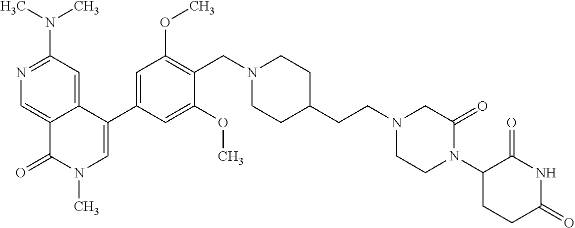 [SMILES: COc1cc(-c2cn(C)c(=O)c3cnc(N(C)C)cc23)cc(OC)c1CN1CCC(CCN2CCN(C3CCC(=O)NC3=O)C(=O)C2)CC1] |
| H89 | 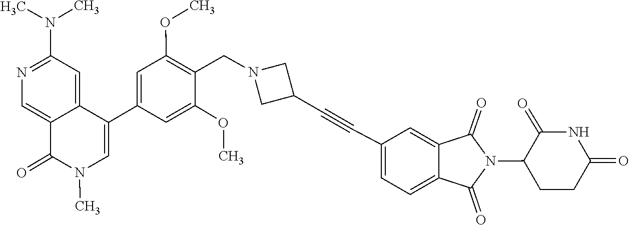 [SMILES: COc1cc(-c2cn(C)c(=O)c3cnc(N(C)C)cc23)cc(OC)c1CN1CC(C#Cc2ccc3c(c2)C(=O)N(C2CCC(=O)NC2=O)C3=O)C1] |
| H90 | 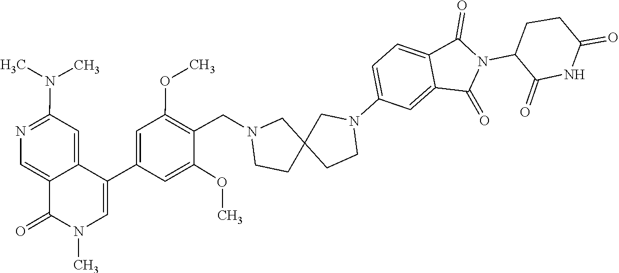 [SMILES: COc1cc(-c2cn(C)c(=O)c3cnc(N(C)C)cc23)cc(OC)c1CN1CCC2(CCN(c3ccc4c(c3)C(=O)N(C3CCC(=O)NC3=O)C4=O)C2)C1] |
| H91 | 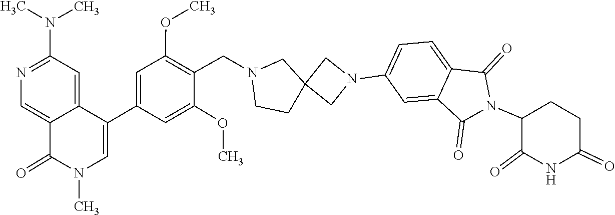 [SMILES: COc1cc(-c2cn(C)c(=O)c3cnc(N(C)C)cc23)cc(OC)c1CN1CCC2(C1)CN(c1ccc3c(c1)C(=O)N(C1CCC(=O)NC1=O)C3=O)C2] |
| H92 | 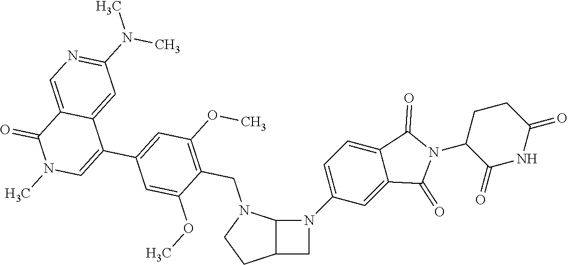 [SMILES: COc1cc(-c2cn(C)c(=O)c3cnc(N(C)C)cc23)cc(OC)c1CN1CCC2CN(c3ccc4c(c3)C(=O)N(C3CCC(=O)NC3=O)C4=O)C21] |
| H93 | 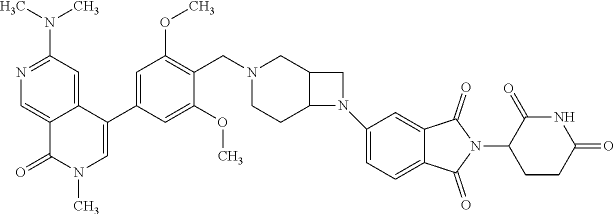 [SMILES: COc1cc(-c2cn(C)c(=O)c3cnc(N(C)C)cc23)cc(OC)c1CN1CCC2C(C1)CN2c1ccc2c(c1)C(=O)N(C1CCC(=O)NC1=O)C2=O] |
| H94 | 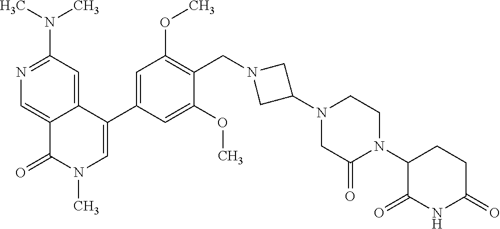 [SMILES: COc1cc(-c2cn(C)c(=O)c3cnc(N(C)C)cc23)cc(OC)c1CN1CC(N2CCN(C3CCC(=O)NC3=O)C(=O)C2)C1] |
| H95 | 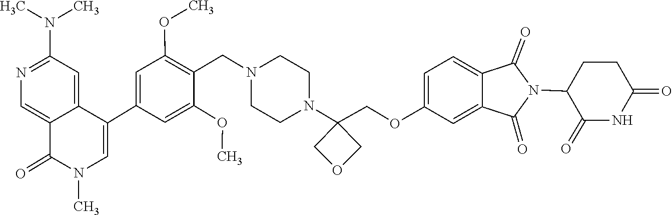 [SMILES: COc1cc(-c2cn(C)c(=O)c3cnc(N(C)C)cc23)cc(OC)c1CN1CCN(C2(COc3ccc4c(c3)C(=O)N(C3CCC(=O)NC3=O)C4=O)COC2)CC1] |
| H96 | 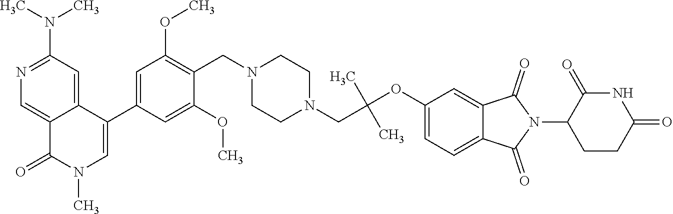 [SMILES: COc1cc(-c2cn(C)c(=O)c3cnc(N(C)C)cc23)cc(OC)c1CN1CCN(CC(C)(C)Oc2ccc3c(c2)C(=O)N(C2CCC(=O)NC2=O)C3=O)CC1] |
| H97 | 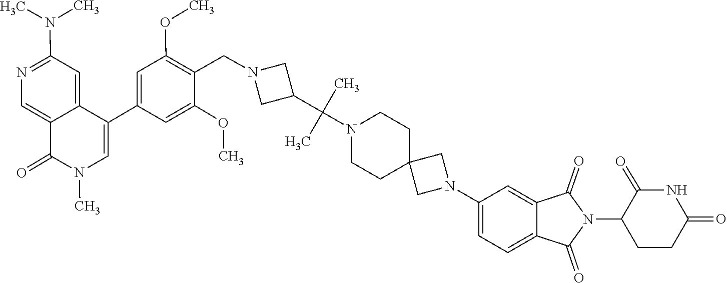 [SMILES: COc1cc(-c2cn(C)c(=O)c3cnc(N(C)C)cc23)cc(OC)c1CN1CC(C(C)(C)N2CCC3(CC2)CN(c2ccc4c(c2)C(=O)N(C2CCC(=O)NC2=O)C4=O)C3)C1] |
| H98 | 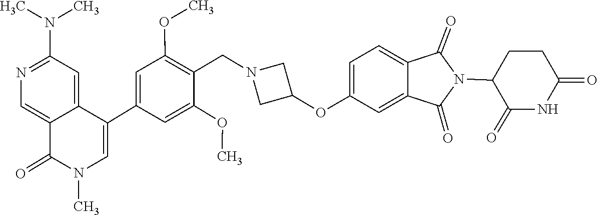 [SMILES: COc1cc(-c2cn(C)c(=O)c3cnc(N(C)C)cc23)cc(OC)c1CN1CC(Oc2ccc3c(c2)C(=O)N(C2CCC(=O)NC2=O)C3=O)C1] |
| H99 | 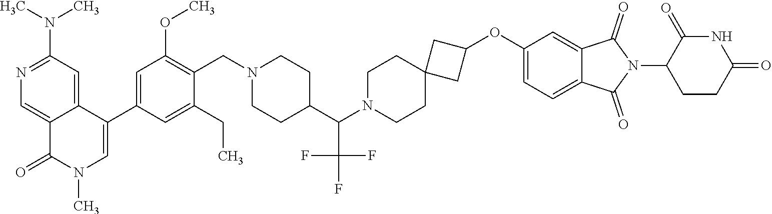 [SMILES: CCc1cc(-c2cn(C)c(=O)c3cnc(N(C)C)cc23)cc(OC)c1CN1CCC(C(N2CCC3(CC2)CC(Oc2ccc4c(c2)C(=O)N(C2CCC(=O)NC2=O)C4=O)C3)C(F)(F)F)CC1] |
| H100 | 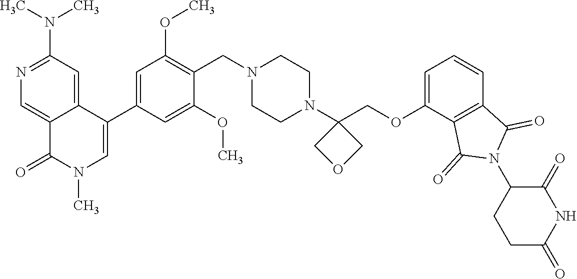 [SMILES: COc1cc(-c2cn(C)c(=O)c3cnc(N(C)C)cc23)cc(OC)c1CN1CCN(C2(COc3cccc4c3C(=O)N(C3CCC(=O)NC3=O)C4=O)COC2)CC1] |
| H101 | 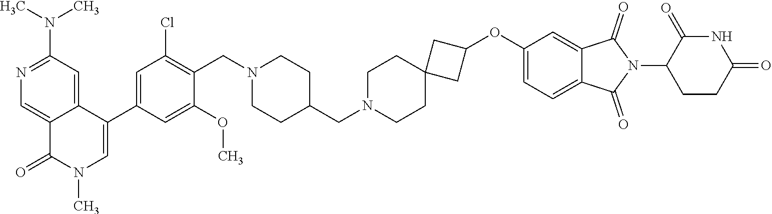 [SMILES: COc1cc(-c2cn(C)c(=O)c3cnc(N(C)C)cc23)cc(Cl)c1CN1CCC(CN2CCC3(CC2)CC(Oc2ccc4c(c2)C(=O)N(C2CCC(=O)NC2=O)C4=O)C3)CC1] |
| H102 | 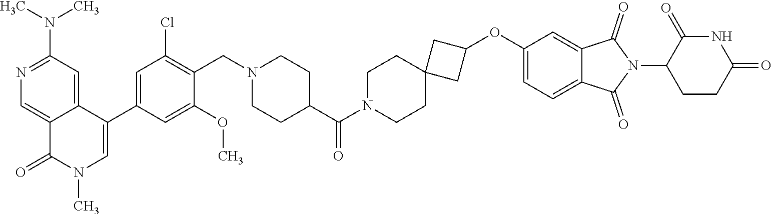 [SMILES: COc1cc(-c2cn(C)c(=O)c3cnc(N(C)C)cc23)cc(Cl)c1CN1CCC(C(=O)N2CCC3(CC2)CC(Oc2ccc4c(c2)C(=O)N(C2CCC(=O)NC2=O)C4=O)C3)CC1] |
| H103 | 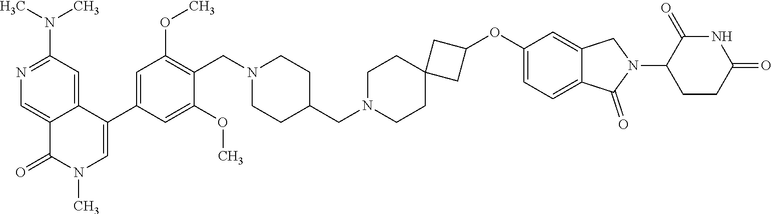 [SMILES: COc1cc(-c2cn(C)c(=O)c3cnc(N(C)C)cc23)cc(OC)c1CN1CCC(CN2CCC3(CC2)CC(Oc2ccc4c(c2)CN(C2CCC(=O)NC2=O)C4=O)C3)CC1] |
| H104 | 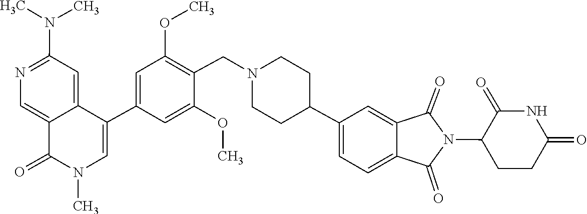 [SMILES: COc1cc(-c2cn(C)c(=O)c3cnc(N(C)C)cc23)cc(OC)c1CN1CCC(c2ccc3c(c2)C(=O)N(C2CCC(=O)NC2=O)C3=O)CC1] |
| H105 | 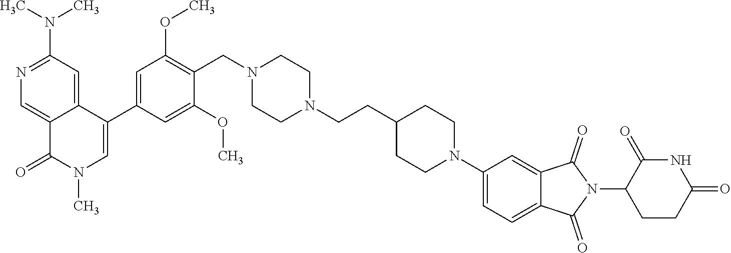 [SMILES: COc1cc(-c2cn(C)c(=O)c3cnc(N(C)C)cc23)cc(OC)c1CN1CCN(CCC2CCN(c3ccc4c(c3)C(=O)N(C3CCC(=O)NC3=O)C4=O)CC2)CC1] |
| H106 | 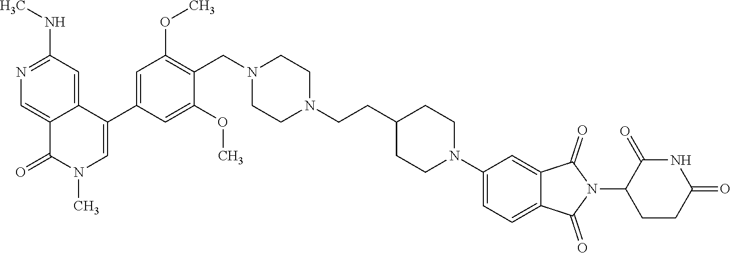 [SMILES: CNc1cc2c(-c3cc(OC)c(CN4CCN(CCC5CCN(c6ccc7c(c6)C(=O)N(C6CCC(=O)NC6=O)C7=O)CC5)CC4)c(OC)c3)cn(C)c(=O)c2cn1] |
| H107 | 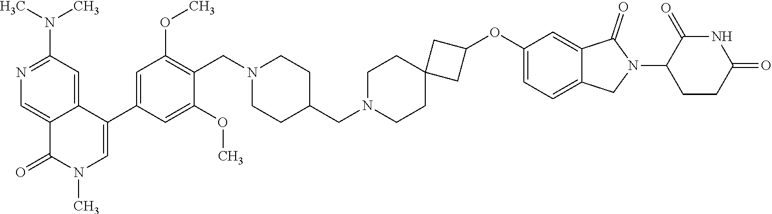 [SMILES: COc1cc(-c2cn(C)c(=O)c3cnc(N(C)C)cc23)cc(OC)c1CN1CCC(CN2CCC3(CC2)CC(Oc2ccc4c(c2)C(=O)N(C2CCC(=O)NC2=O)C4)C3)CC1] |
| H108 | 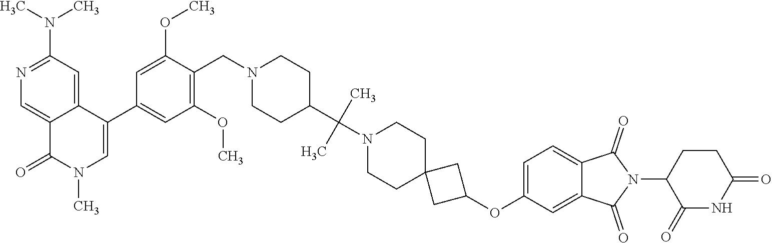 [SMILES: COc1cc(-c2cn(C)c(=O)c3cnc(N(C)C)cc23)cc(OC)c1CN1CCC(C(C)(C)N2CCC3(CC2)CC(Oc2ccc4c(c2)C(=O)N(C2CCC(=O)NC2=O)C4=O)C3)CC1] |
| H109 | 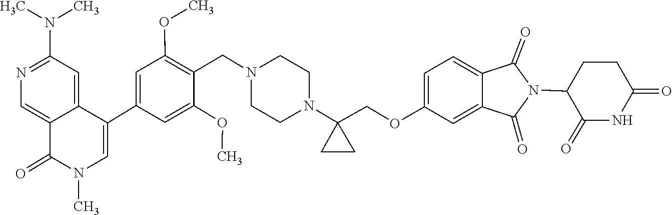 [SMILES: COc1cc(-c2cn(C)c(=O)c3cnc(N(C)C)cc23)cc(OC)c1CN1CCN(C2(COc3ccc4c(c3)C(=O)N(C3CCC(=O)NC3=O)C4=O)CC2)CC1] |
| H110 | 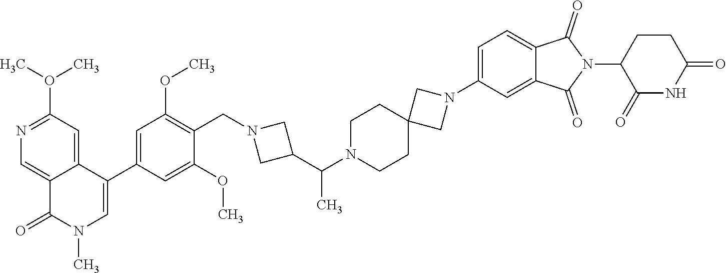 |
| H111 | 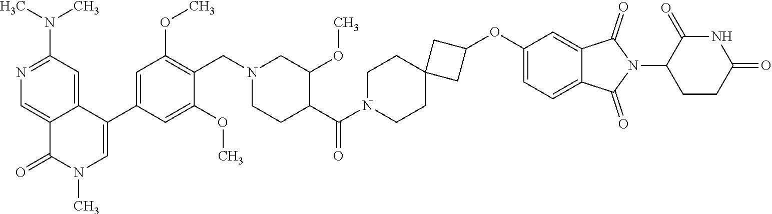 [SMILES: COc1cc(-c2cn(C)c(=O)c3cnc(N(C)C)cc23)cc(OC)c1CN1CCC(C(=O)N2CCC3(CC2)CC(Oc2ccc4c(c2)C(=O)N(C2CCC(=O)NC2=O)C4=O)C3)C(OC)C1] |
| H112 | 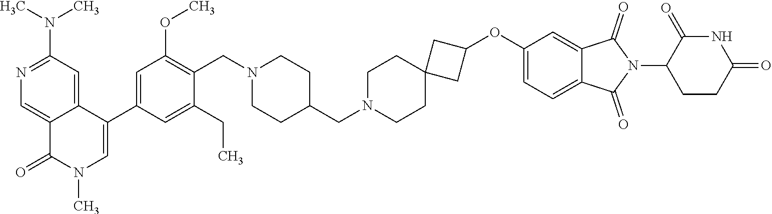 [SMILES: CCc1cc(-c2cn(C)c(=O)c3cnc(N(C)C)cc23)cc(OC)c1CN1CCC(CN2CCC3(CC2)CC(Oc2ccc4c(c2)C(=O)N(C2CCC(=O)NC2=O)C4=O)C3)CC1] |
| H113 | 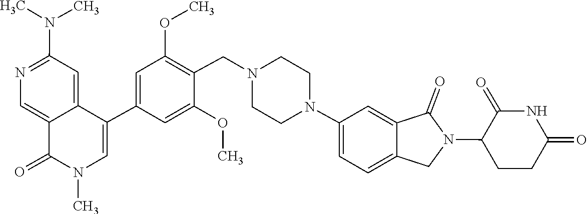 [SMILES: COc1cc(-c2cn(C)c(=O)c3cnc(N(C)C)cc23)cc(OC)c1CN1CCN(c2ccc3c(c2)C(=O)N(C2CCC(=O)NC2=O)C3)CC1] |
| H114 | 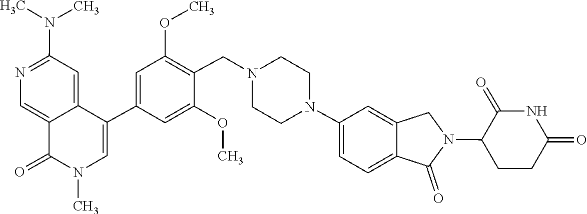 [SMILES: COc1cc(-c2cn(C)c(=O)c3cnc(N(C)C)cc23)cc(OC)c1CN1CCN(c2ccc3c(c2)CN(C2CCC(=O)NC2=O)C3=O)CC1] |
| H115 | 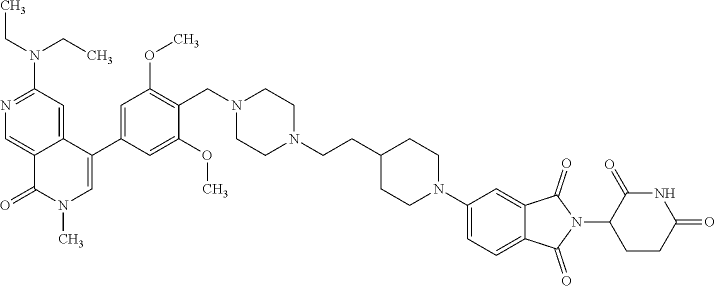 [SMILES: CCN(CC)c1cc2c(-c3cc(OC)c(CN4CCN(CCC5CCN(c6ccc7c(c6)C(=O)N(C6CCC(=O)NC6=O)C7=O)CC5)CC4)c(OC)c3)cn(C)c(=O)c2cn1] |
| H116 | 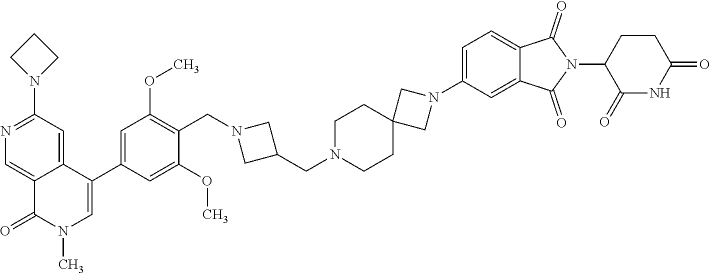 [SMILES: COc1cc(-c2cn(C)c(=O)c3cnc(N4CCC4)cc23)cc(OC)c1CN1CC(CN2CCC3(CC2)CN(c2ccc4c(c2)C(=O)N(C2CCC(=O)NC2=O)C4=O)C3)C1] |
| H117 | 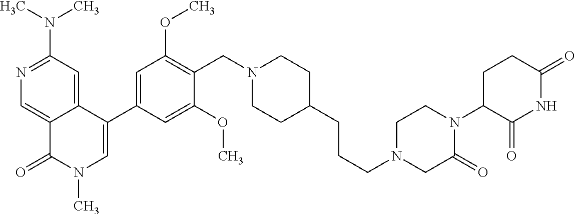 [SMILES: COc1cc(-c2cn(C)c(=O)c3cnc(N(C)C)cc23)cc(OC)c1CN1CCC(CCCN2CCN(C3CCC(=O)NC3=O)C(=O)C2)CC1] |
| H118 | 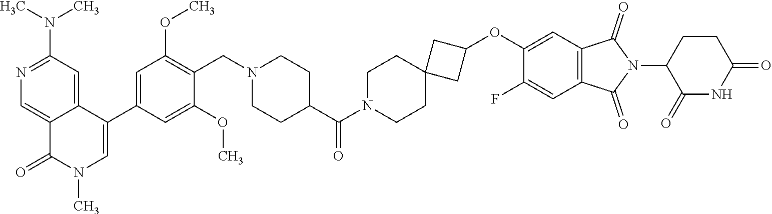 [SMILES: COc1cc(-c2cn(C)c(=O)c3cnc(N(C)C)cc23)cc(OC)c1CN1CCC(C(=O)N2CCC3(CC2)CC(Oc2cc4c(cc2F)C(=O)N(C2CCC(=O)NC2=O)C4=O)C3)CC1] |
| H119 | 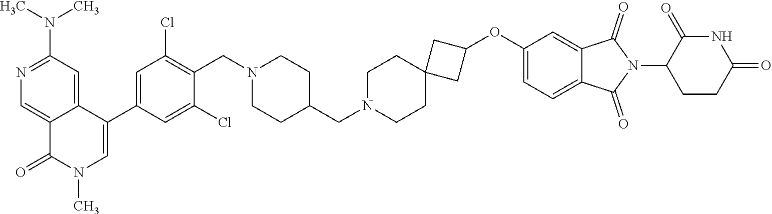 [SMILES: CN(C)c1cc2c(-c3cc(Cl)c(CN4CCC(CN5CCC6(CC5)CC(Oc5ccc7c(c5)C(=O)N(C5CCC(=O)NC5=O)C7=O)C6)CC4)c(Cl)c3)cn(C)c(=O)c2cn1] |
| H120 | 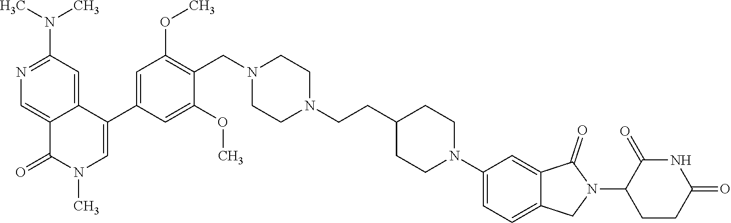 [SMILES: COc1cc(-c2cn(C)c(=O)c3cnc(N(C)C)cc23)cc(OC)c1CN1CCN(CCC2CCN(c3ccc4c(c3)C(=O)N(C3CCC(=O)NC3=O)C4)CC2)CC1] |
| H121 | 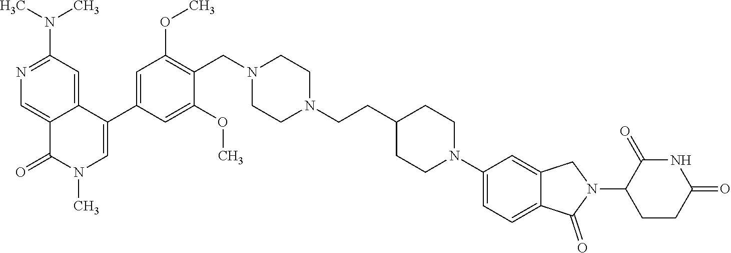 [SMILES: COc1cc(-c2cn(C)c(=O)c3cnc(N(C)C)cc23)cc(OC)c1CN1CCN(CCC2CCN(c3ccc4c(c3)CN(C3CCC(=O)NC3=O)C4=O)CC2)CC1] |
| H122 | 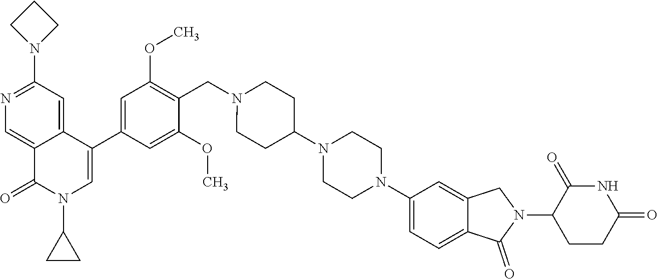 [SMILES: COc1cc(-c2cn(C3CC3)c(=O)c3cnc(N4CCC4)cc23)cc(OC)c1CN1CCC(N2CCN(c3ccc4c(c3)CN(C3CCC(=O)NC3=O)C4=O)CC2)CC1] |
| H123 | 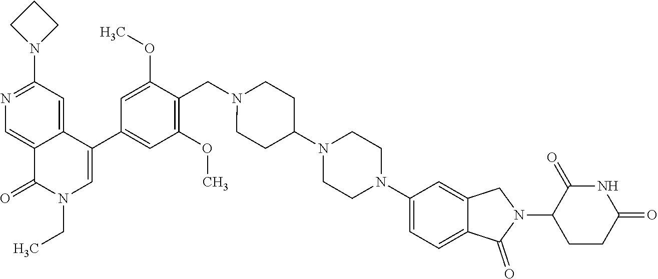 [SMILES: CCn1cc(-c2cc(OC)c(CN3CCC(N4CCN(c5ccc6c(c5)CN(C5CCC(=O)NC5=O)C6=O)CC4)CC3)c(OC)c2)c2cc(N3CCC3)ncc2c1=O] |
| H124 | 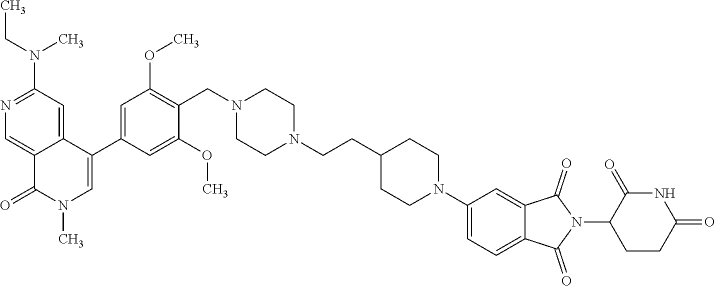 [SMILES: CCN(C)c1cc2c(-c3cc(OC)c(CN4CCN(CCC5CCN(c6ccc7c(c6)C(=O)N(C6CCC(=O)NC6=O)C7=O)CC5)CC4)c(OC)c3)cn(C)c(=O)c2cn1] |
| H125 | 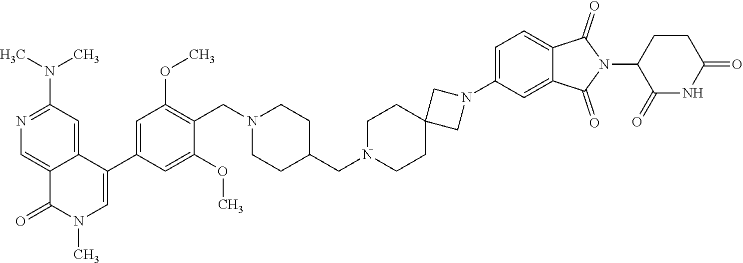 [SMILES: COc1cc(-c2cn(C)c(=O)c3cnc(N(C)C)cc23)cc(OC)c1CN1CCC(CN2CCC3(CC2)CN(c2ccc4c(c2)C(=O)N(C2CCC(=O)NC2=O)C4=O)C3)CC1] |
| H126 | 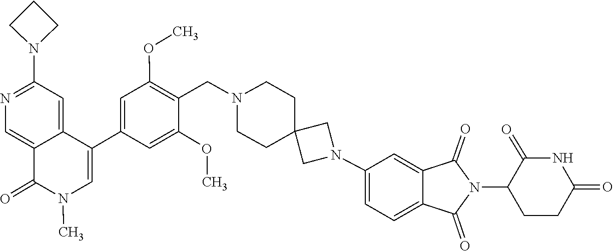 [SMILES: COc1cc(-c2cn(C)c(=O)c3cnc(N4CCC4)cc23)cc(OC)c1CN1CCC2(CC1)CN(c1ccc3c(c1)C(=O)N(C1CCC(=O)NC1=O)C3=O)C2] |
| H127 | 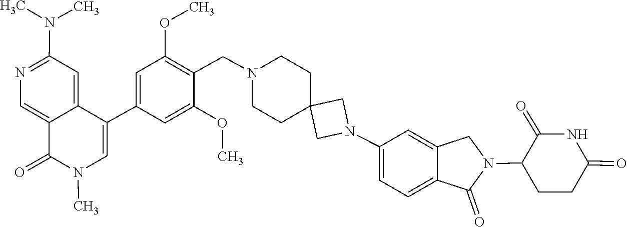 [SMILES: COc1cc(-c2cn(C)c(=O)c3cnc(N(C)C)cc23)cc(OC)c1CN1CCC2(CC1)CN(c1ccc3c(c1)CN(C1CCC(=O)NC1=O)C3=O)C2] |
| H128 | 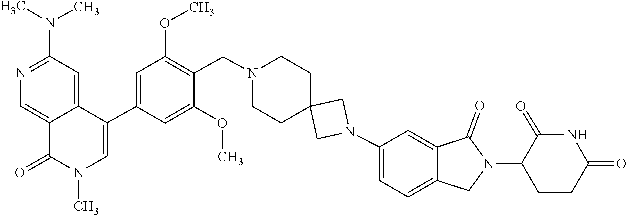 [SMILES: COc1cc(-c2cn(C)c(=O)c3cnc(N(C)C)cc23)cc(OC)c1CN1CCC2(CC1)CN(c1ccc3c(c1)C(=O)N(C1CCC(=O)NC1=O)C3)C2] |
| H129 | 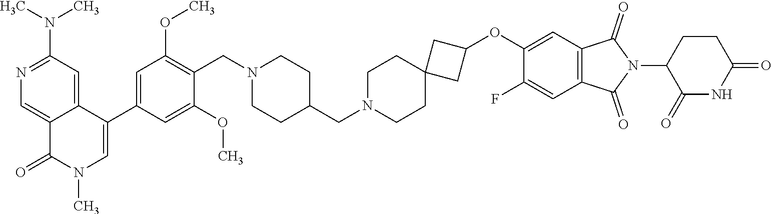 [SMILES: COc1cc(-c2cn(C)c(=O)c3cnc(N(C)C)cc23)cc(OC)c1CN1CCC(CN2CCC3(CC2)CC(Oc2cc4c(cc2F)C(=O)N(C2CCC(=O)NC2=O)C4=O)C3)CC1] |
| H130 | 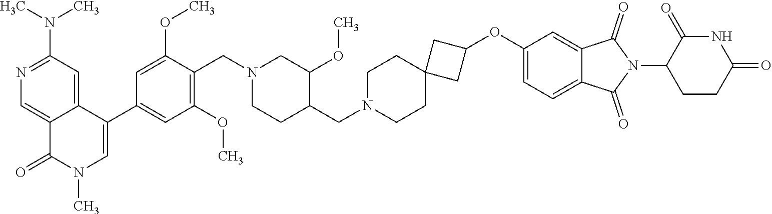 [SMILES: COc1cc(-c2cn(C)c(=O)c3cnc(N(C)C)cc23)cc(OC)c1CN1CCC(CN2CCC3(CC2)CC(Oc2ccc4c(c2)C(=O)N(C2CCC(=O)NC2=O)C4=O)C3)C(OC)C1] |
| H131 | 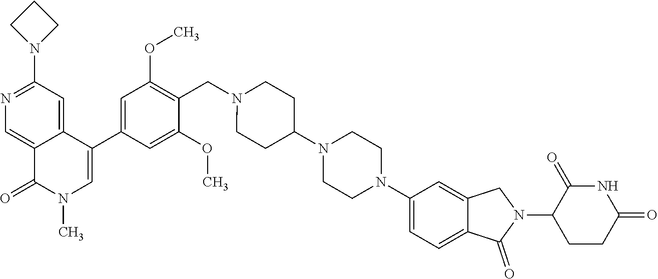 [SMILES: COc1cc(-c2cn(C)c(=O)c3cnc(N4CCC4)cc23)cc(OC)c1CN1CCC(N2CCN(c3ccc4c(c3)CN(C3CCC(=O)NC3=O)C4=O)CC2)CC1] |
| H132 | 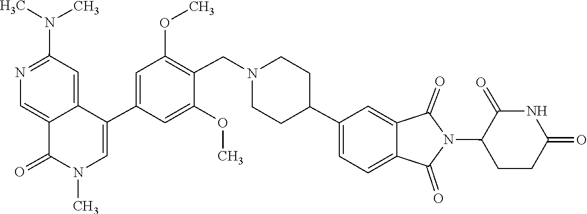 [SMILES: COc1cc(-c2cn(C)c(=O)c3cnc(N(C)C)cc23)cc(OC)c1CN1CCC(c2ccc3c(c2)C(=O)N(C2CCC(=O)NC2=O)C3=O)CC1] |
| H133 | 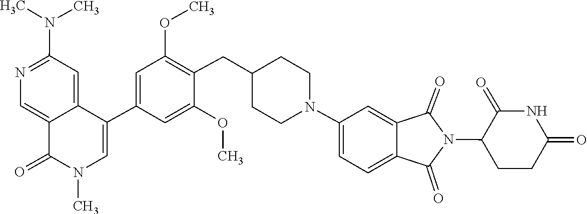 [SMILES: COc1cc(-c2cn(C)c(=O)c3cnc(N(C)C)cc23)cc(OC)c1CC1CCN(c2ccc3c(c2)C(=O)N(C2CCC(=O)NC2=O)C3=O)CC1] |
| H134 | 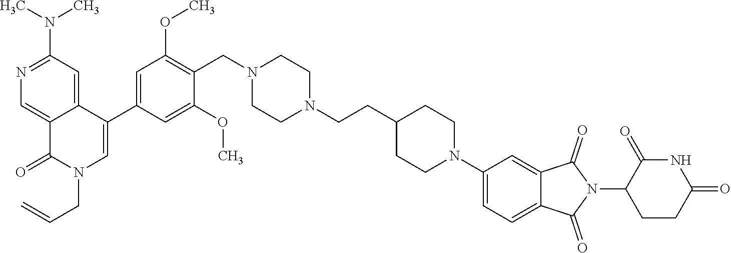 [SMILES: C=CCn1cc(-c2cc(OC)c(CN3CCN(CCC4CCN(c5ccc6c(c5)C(=O)N(C5CCC(=O)NC5=O)C6=O)CC4)CC3)c(OC)c2)c2cc(N(C)C)ncc2c1=O] |
| H135 | 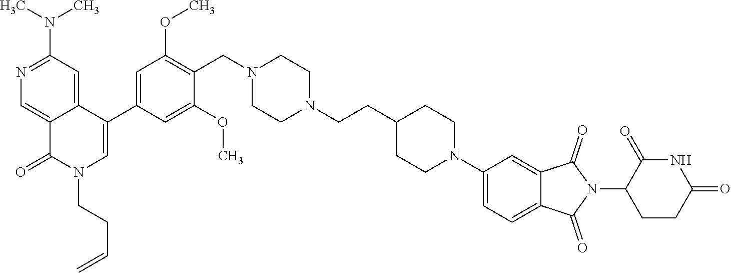 [SMILES: C=CCCn1cc(-c2cc(OC)c(CN3CCN(CCC4CCN(c5ccc6c(c5)C(=O)N(C5CCC(=O)NC5=O)C6=O)CC4)CC3)c(OC)c2)c2cc(N(C)C)ncc2c1=O] |
| H136 | 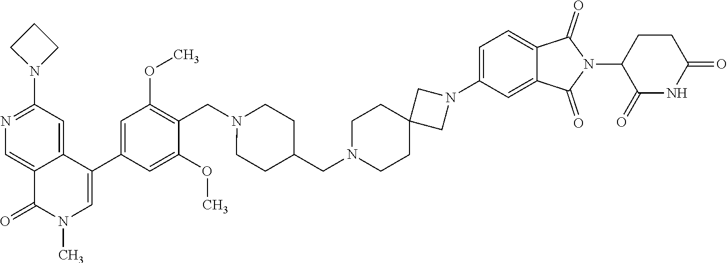 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4=O)C3)CC1] |
| H137 | 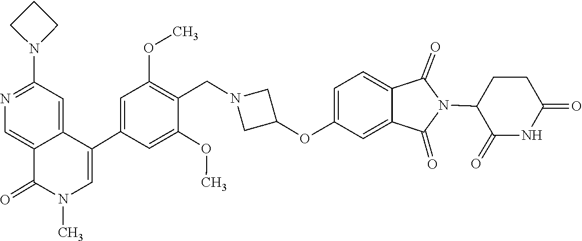 [SMILES: COc1cc(-c2cn(C)c(=O)c3cnc(N4CCC4)cc23)cc(OC)c1CN1CC(Oc2ccc3c(c2)C(=O)N(C2CCC(=O)NC2=O)C3=O)C1] |
| H138 | 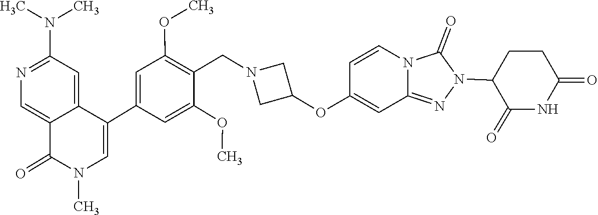 [SMILES: COc1cc(-c2cn(C)c(=O)c3cnc(N(C)C)cc23)cc(OC)c1CN1CC(Oc2ccn3c(=O)n(C4CCC(=O)NC4=O)nc3c2)C1] |
| H139 | 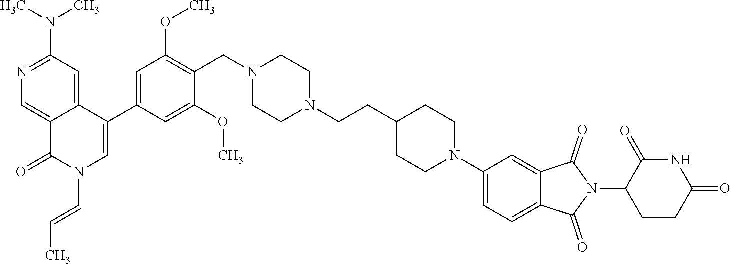 [SMILES: C/C=C/n1cc(-c2cc(OC)c(CN3CCN(CCC4CCN(c5ccc6c(c5)C(=O)N(C5CCC(=O)NC5=O)C6=O)CC4)CC3)c(OC)c2)c2cc(N(C)C)ncc2c1=O] |
| H140 | 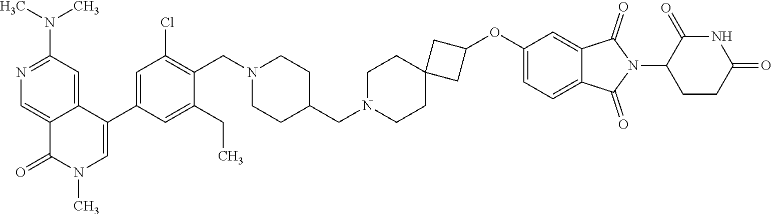 [SMILES: CCc1cc(-c2cn(C)c(=O)c3cnc(N(C)C)cc23)cc(Cl)c1CN1CCC(CN2CCC3(CC2)CC(Oc2ccc4c(c2)C(=O)N(C2CCC(=O)NC2=O)C4=O)C3)CC1] |
| H141 | 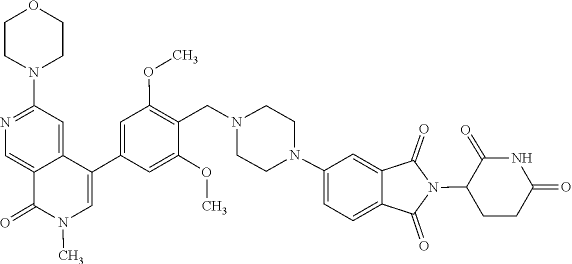 [SMILES: COc1cc(-c2cn(C)c(=O)c3cnc(N4CCOCC4)cc23)cc(OC)c1CN1CCN(c2ccc3c(c2)C(=O)N(C2CCC(=O)NC2=O)C3=O)CC1] |
| H142 | 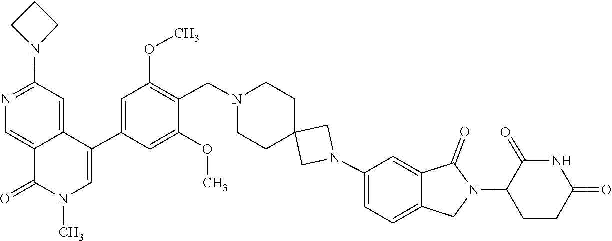 [SMILES: COc1cc(-c2cn(C)c(=O)c3cnc(N4CCC4)cc23)cc(OC)c1CN1CCC2(CC1)CN(c1ccc3c(c1)C(=O)N(C1CCC(=O)NC1=O)C3)C2] |
| H143 | 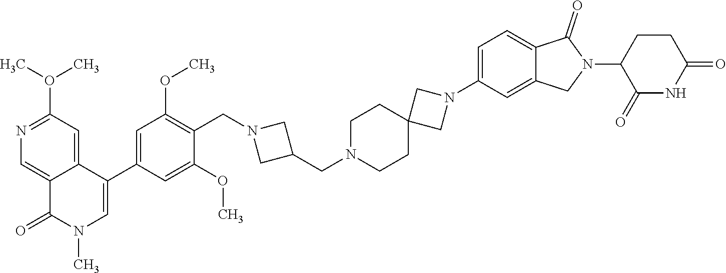 |
| H144 | 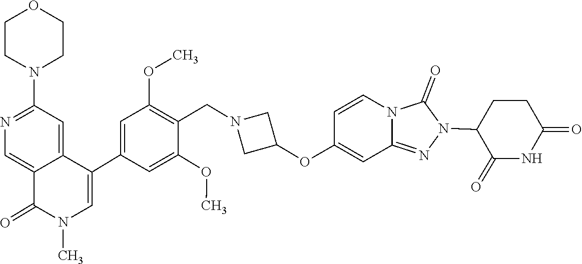 [SMILES: COc1cc(-c2cn(C)c(=O)c3cnc(N4CCOCC4)cc23)cc(OC)c1CN1CC(Oc2ccn3c(=O)n(C4CCC(=O)NC4=O)nc3c2)C1] |
| H145 | 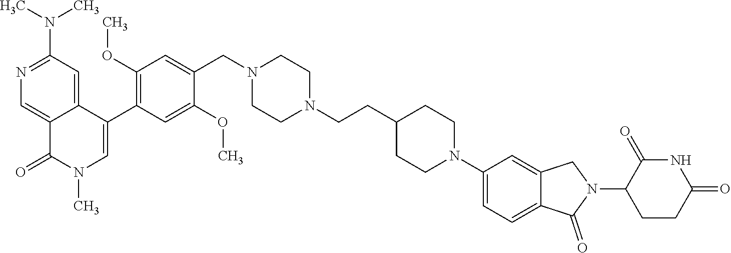 [SMILES: COc1cc(-c2cn(C)c(=O)c3cnc(N(C)C)cc23)c(OC)cc1CN1CCN(CCC2CCN(c3ccc4c(c3)CN(C3CCC(=O)NC3=O)C4=O)CC2)CC1] |
| H146 | 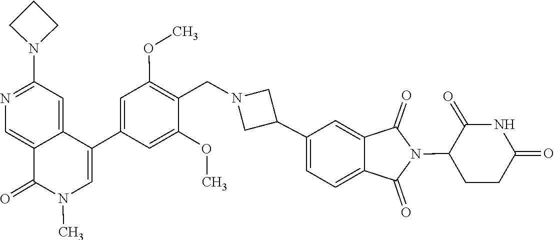 [SMILES: COc1cc(-c2cn(C)c(=O)c3cnc(N4CCC4)cc23)cc(OC)c1CN1CC(c2ccc3c(c2)C(=O)N(C2CCC(=O)NC2=O)C3=O)C1] |
| H147 | 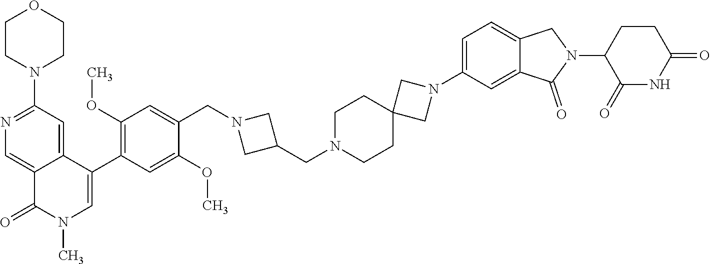 [SMILES: COc1cc(-c2cn(C)c(=O)c3cnc(N4CCOCC4)cc23)c(OC)cc1CN1CC(CN2CCC3(CC2)CN(c2ccc4c(c2)C(=O)N(C2CCC(=O)NC2=O)C4)C3)C1] |
| H148 | 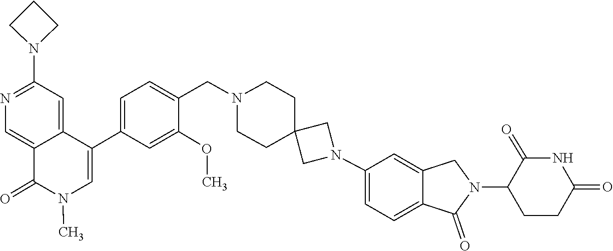 [SMILES: COc1cc(-c2cn(C)c(=O)c3cnc(N4CCC4)cc23)ccc1CN1CCC2(CC1)CN(c1ccc3c(c1)CN(C1CCC(=O)NC1=O)C3=O)C2] |
| H149 | 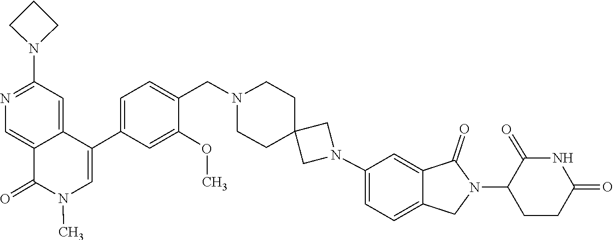 [SMILES: COc1cc(-c2cn(C)c(=O)c3cnc(N4CCC4)cc23)ccc1CN1CCC2(CC1)CN(c1ccc3c(c1)C(=O)N(C1CCC(=O)NC1=O)C3)C2] |
| H150 | 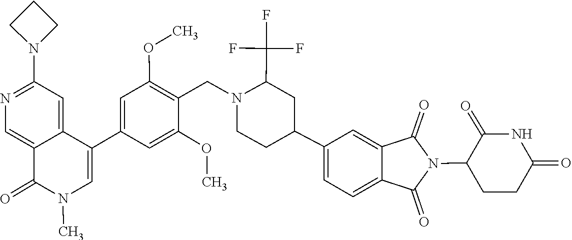 [SMILES: COc1cc(-c2cn(C)c(=O)c3cnc(N4CCC4)cc23)cc(OC)c1CN1CCC(c2ccc3c(c2)C(=O)N(C2CCC(=O)NC2=O)C3=O)CC1C(F)(F)F] |
| H151 | 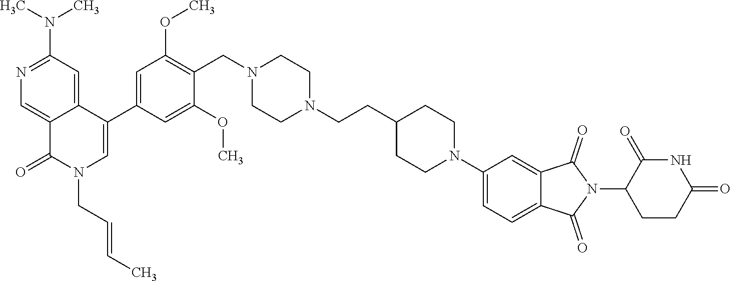 [SMILES: C/C=C/Cn1cc(-c2cc(OC)c(CN3CCN(CCC4CCN(c5ccc6c(c5)C(=O)N(C5CCC(=O)NC5=O)C6=O)CC4)CC3)c(OC)c2)c2cc(N(C)C)ncc2c1=O] |
| H152 | 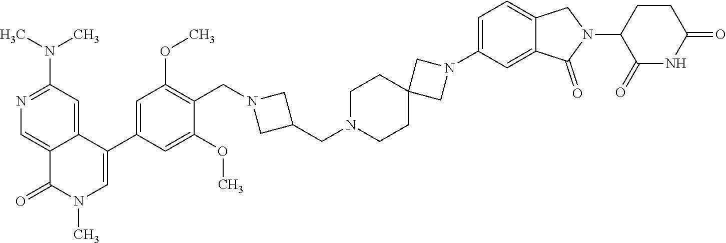 [SMILES: COc1cc(-c2cn(C)c(=O)c3cnc(N(C)C)cc23)cc(OC)c1CN1CC(CN2CCC3(CC2)CN(c2ccc4c(c2)C(=O)N(C2CCC(=O)NC2=O)C4)C3)C1] |
| H153 | 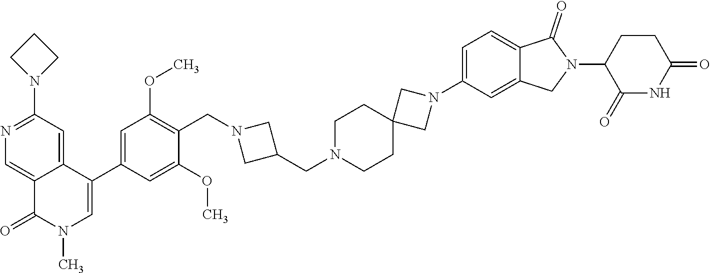 [SMILES: COc1cc(-c2cn(C)c(=O)c3cnc(N4CCC4)cc23)cc(OC)c1CN1CC(CN2CCC3(CC2)CN(c2ccc4c(c2)CN(C2CCC(=O)NC2=O)C4=O)C3)C1] |
| H154 | 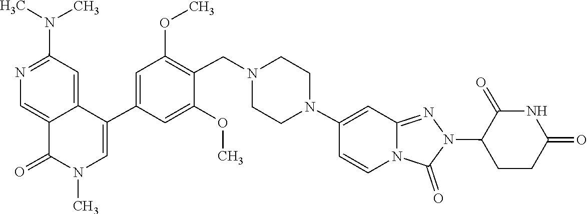 [SMILES: COc1cc(-c2cn(C)c(=O)c3cnc(N(C)C)cc23)cc(OC)c1CN1CCN(c2ccn3c(=O)n(C4CCC(=O)NC4=O)nc3c2)CC1] |
| H155 | 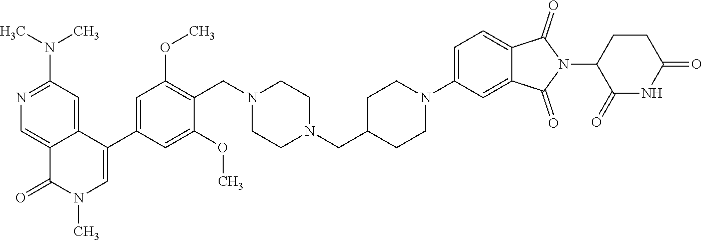 [SMILES: COc1cc(-c2cn(C)c(=O)c3cnc(N(C)C)cc23)cc(OC)c1CN1CCN(CC2CCN(c3ccc4c(c3)C(=O)N(C3CCC(=O)NC3=O)C4=O)CC2)CC1] |
| H156 | 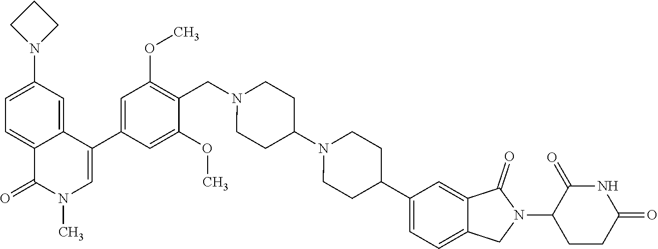 [SMILES: COc1cc(-c2cn(C)c(=O)c3ccc(N4CCC4)cc23)cc(OC)c1CN1CCC(N2CCC(c3ccc4c(c3)C(=O)N(C3CCC(=O)NC3=O)C4)CC2)CC1] |
| H157 | 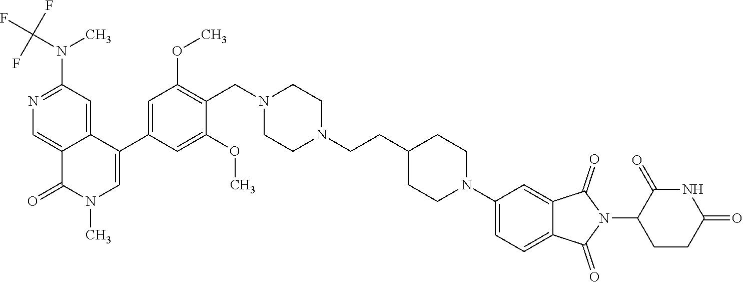 [SMILES: COc1cc(-c2cn(C)c(=O)c3cnc(N(C)C(F)(F)F)cc23)cc(OC)c1CN1CCN(CCC2CCN(c3ccc4c(c3)C(=O)N(C3CCC(=O)NC3=O)C4=O)CC2)CC1] |
| H158 | 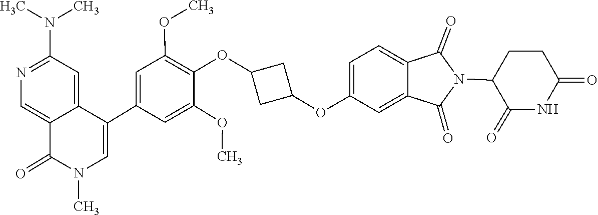 [SMILES: COc1cc(-c2cn(C)c(=O)c3cnc(N(C)C)cc23)cc(OC)c1OC1CC(Oc2ccc3c(c2)C(=O)N(C2CCC(=O)NC2=O)C3=O)C1] |
| H159 | 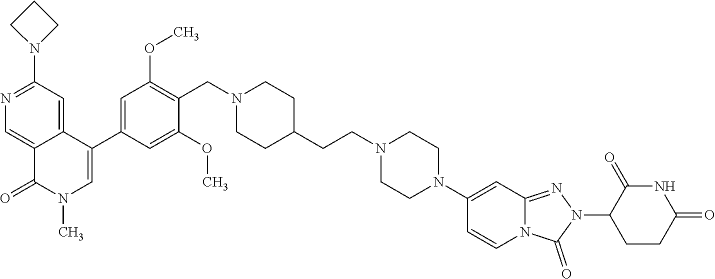 [SMILES: COc1cc(-c2cn(C)c(=O)c3cnc(N4CCC4)cc23)cc(OC)c1CN1CCC(CCN2CCN(c3ccn4c(=O)n(C5CCC(=O)NC5=O)nc4c3)CC2)CC1] |
| H160 | 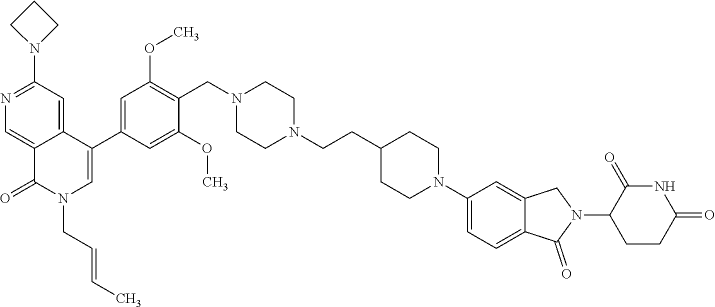 [SMILES: C/C=C/Cn1cc(-c2cc(OC)c(CN3CCN(CCC4CCN(c5ccc6c(c5)CN(C5CCC(=O)NC5=O)C6=O)CC4)CC3)c(OC)c2)c2cc(N3CCC3)ncc2c1=O] |
| H161 | 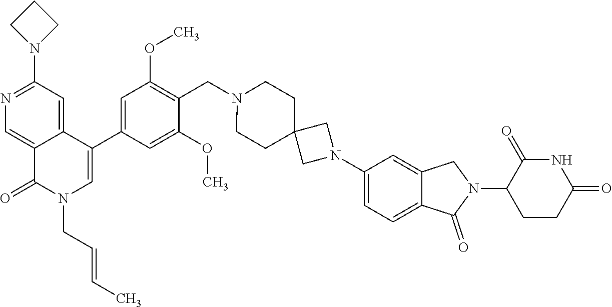 [SMILES: C/C=C/Cn1cc(-c2cc(OC)c(CN3CCC4(CC3)CN(c3ccc5c(c3)CN(C3CCC(=O)NC3=O)C5=O)C4)c(OC)c2)c2cc(N3CCC3)ncc2c1=O] |
| H162 | 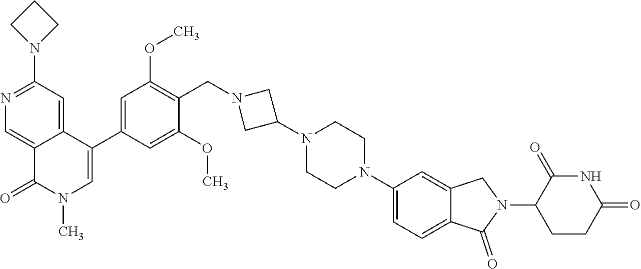 [SMILES: COc1cc(-c2cn(C)c(=O)c3cnc(N4CCC4)cc23)cc(OC)c1CN1CC(N2CCN(c3ccc4c(c3)CN(C3CCC(=O)NC3=O)C4=O)CC2)C1] |
| H163 | 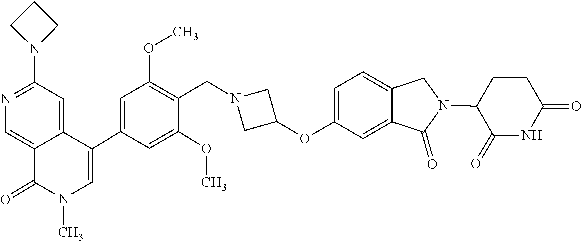 [SMILES: COc1cc(-c2cn(C)c(=O)c3cnc(N4CCC4)cc23)cc(OC)c1CN1CC(Oc2ccc3c(c2)C(=O)N(C2CCC(=O)NC2=O)C3)C1] |
| H164 | 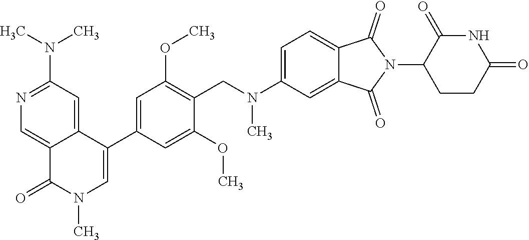 [SMILES: COc1cc(-c2cn(C)c(=O)c3cnc(N(C)C)cc23)cc(OC)c1CN(C)c1ccc2c(c1)C(=O)N(C1CCC(=O)NC1=O)C2=O] |
| H165 | 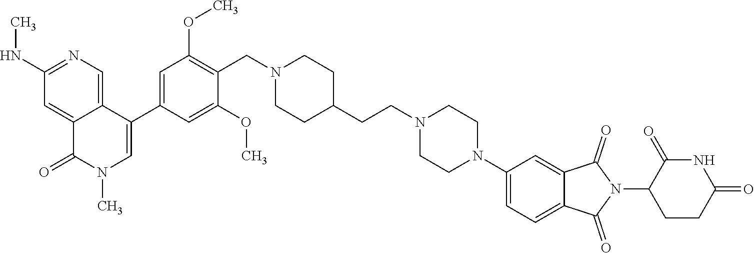 [SMILES: CNc1cc2c(=O)n(C)cc(-c3cc(OC)c(CN4CCC(CCN5CCN(c6ccc7c(c6)C(=O)N(C6CCC(=O)NC6=O)C7=O)CC5)CC4)c(OC)c3)c2cn1] |
| H166 | 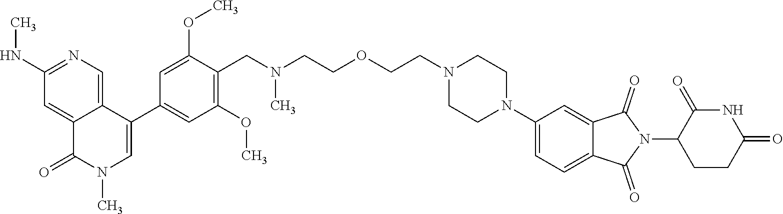 [SMILES: CNc1cc2c(=O)n(C)cc(-c3cc(OC)c(CN(C)CCOCCN4CCN(c5ccc6c(c5)C(=O)N(C5CCC(=O)NC5=O)C6=O)CC4)c(OC)c3)c2cn1] |
| H167 | 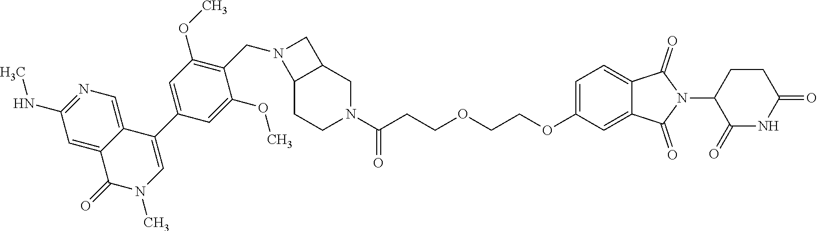 [SMILES: CNc1cc2c(=O)n(C)cc(-c3cc(OC)c(CN4CC5CN(C(=O)CCOCCOc6ccc7c(c6)C(=O)N(C6CCC(=O)NC6=O)C7=O)CCC54)c(OC)c3)c2cn1] |
| H168 | 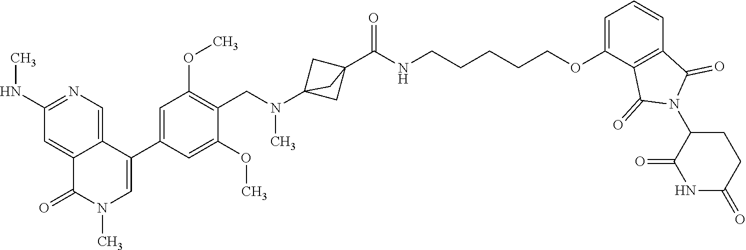 [SMILES: CNc1cc2c(=O)n(C)cc(-c3cc(OC)c(CN(C)C45CC(C(=O)NCCCCCOc6cccc7c6C(=O)N(C6CCC(=O)NC6=O)C7=O)(C4)C5)c(OC)c3)c2cn1] |
| H169 | 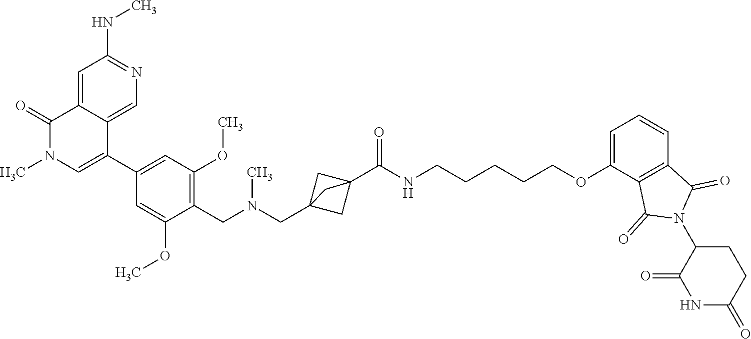 [SMILES: CNc1cc2c(=O)n(C)cc(-c3cc(OC)c(CN(C)CC45CC(C(=O)NCCCCCOc6cccc7c6C(=O)N(C6CCC(=O)NC6=O)C7=O)(C4)C5)c(OC)c3)c2cn1] |
| H170 | 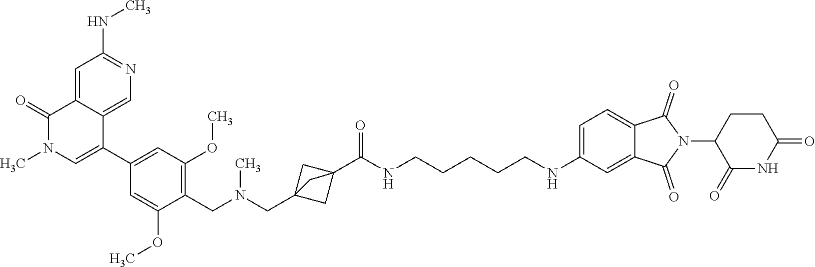 [SMILES: CNc1cc2c(=O)n(C)cc(-c3cc(OC)c(CN(C)CC45CC(C(=O)NCCCCCNc6ccc7c(c6)C(=O)N(C6CCC(=O)NC6=O)C7=O)(C4)C5)c(OC)c3)c2cn1] |
| H171 | 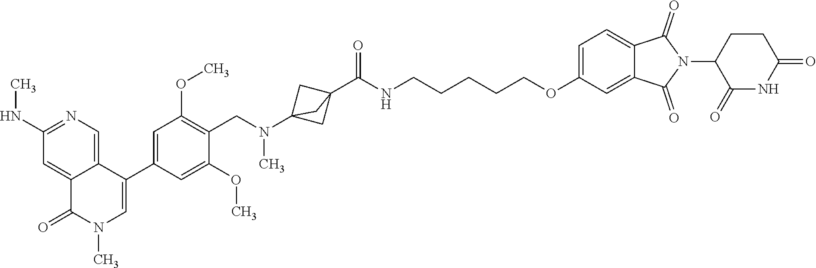 [SMILES: CNc1cc2c(=O)n(C)cc(-c3cc(OC)c(CN(C)C45CC(C(=O)NCCCCCOc6ccc7c(c6)C(=O)N(C6CCC(=O)NC6=O)C7=O)(C4)C5)c(OC)c3)c2cn1] |
| H172 | 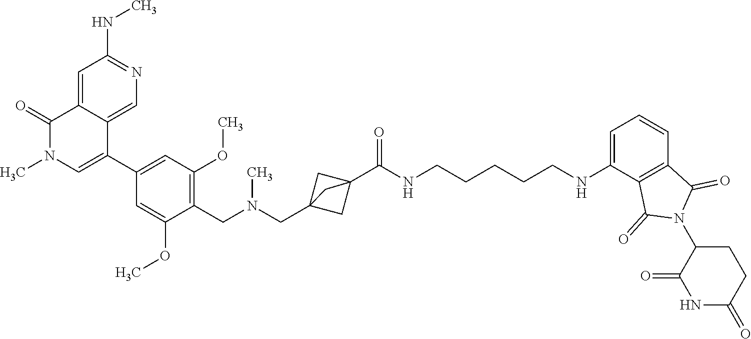 [SMILES: CNc1cc2c(=O)n(C)cc(-c3cc(OC)c(CN(C)CC45CC(C(=O)NCCCCCNc6cccc7c6C(=O)N(C6CCC(=O)NC6=O)C7=O)(C4)C5)c(OC)c3)c2cn1] |
| H173 | 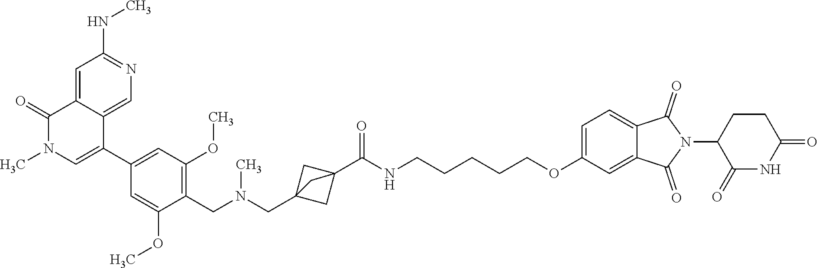 [SMILES: CNc1cc2c(=O)n(C)cc(-c3cc(OC)c(CN(C)CC45CC(C(=O)NCCCCCOc6ccc7c(c6)C(=O)N(C6CCC(=O)NC6=O)C7=O)(C4)C5)c(OC)c3)c2cn1] |
| H174 | 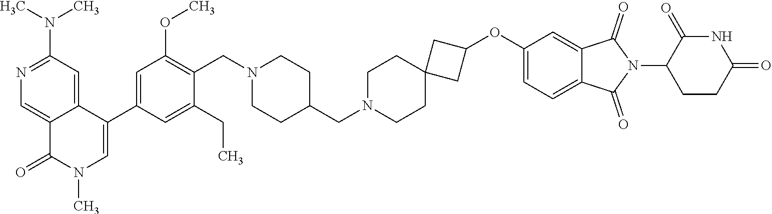 [SMILES: CCc1cc(-c2cn(C)c(=O)c3cnc(N(C)C)cc23)cc(OC)c1CN1CCC(CN2CCC3(CC2)CC(Oc2ccc4c(c2)C(=O)N(C2CCC(=O)NC2=O)C4=O)C3)CC1] |
| H175 | 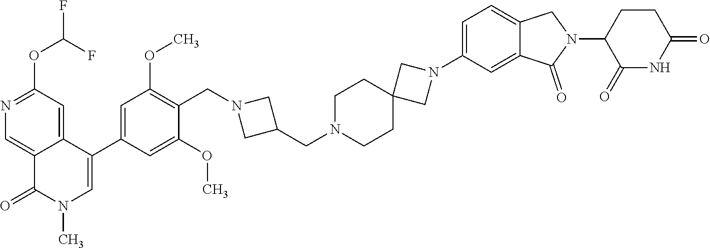 [SMILES: COc1cc(-c2cn(C)c(=O)c3cnc(OC(F)F)cc23)cc(OC)c1CN1CC(CN2CCC3(CC2)CN(c2ccc4c(c2)C(=O)N(C2CCC(=O)NC2=O)C4)C3)C1] |
| H176 | 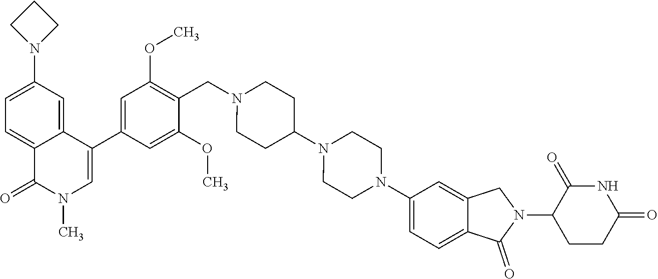 [SMILES: COc1cc(-c2cn(C)c(=O)c3ccc(N4CCC4)cc23)cc(OC)c1CN1CCC(N2CCN(c3ccc4c(c3)CN(C3CCC(=O)NC3=O)C4=O)CC2)CC1] |
| H177 | 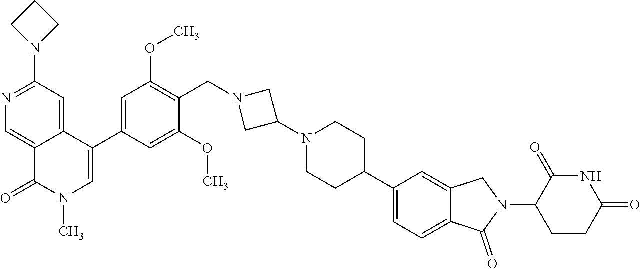 [SMILES: COc1cc(-c2cn(C)c(=O)c3cnc(N4CCC4)cc23)cc(OC)c1CN1CC(N2CCC(c3ccc4c(c3)CN(C3CCC(=O)NC3=O)C4=O)CC2)C1] |
| H178 | 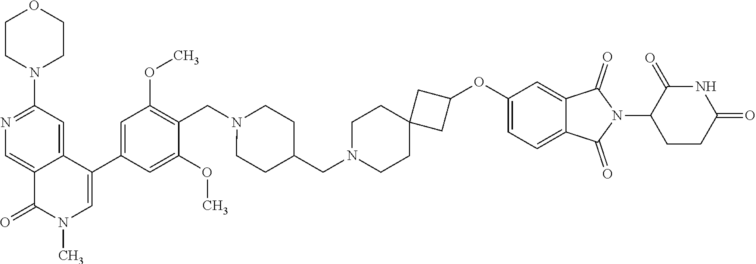 [SMILES: COc1cc(-c2cn(C)c(=O)c3cnc(N4CCOCC4)cc23)cc(OC)c1CN1CCC(CN2CCC3(CC2)CC(Oc2ccc4c(c2)C(=O)N(C2CCC(=O)NC2=O)C4=O)C3)CC1] |
| H179 | 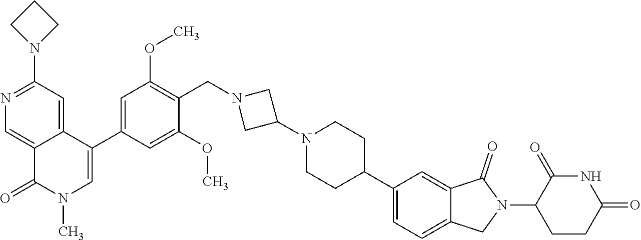 [SMILES: COc1cc(-c2cn(C)c(=O)c3cnc(N4CCC4)cc23)cc(OC)c1CN1CC(N2CCC(c3ccc4c(c3)C(=O)N(C3CCC(=O)NC3=O)C4)CC2)C1] |
| H180 | 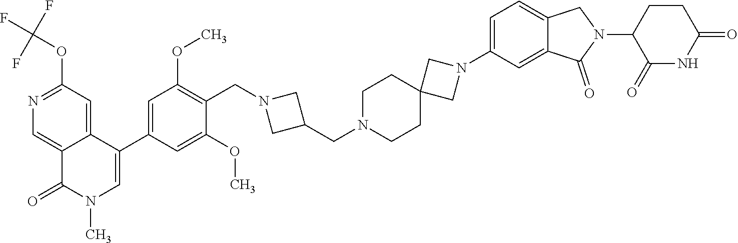 [SMILES: COc1cc(-c2cn(C)c(=O)c3cnc(OC(F)(F)F)cc23)cc(OC)c1CN1CC(CN2CCC3(CC2)CN(c2ccc4c(c2)C(=O)N(C2CCC(=O)NC2=O)C4)C3)C1] |
| H181 | 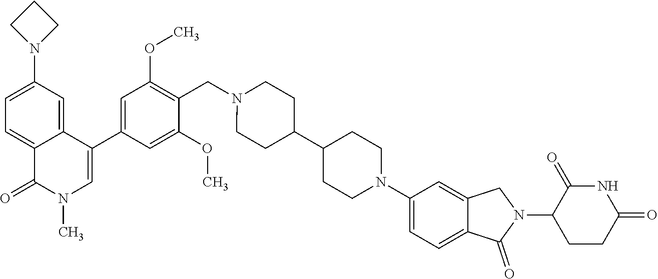 [SMILES: COc1cc(-c2cn(C)c(=O)c3ccc(N4CCC4)cc23)cc(OC)c1CN1CCC(C2CCN(c3ccc4c(c3)CN(C3CCC(=O)NC3=O)C4=O)CC2)CC1] |
| H182 | 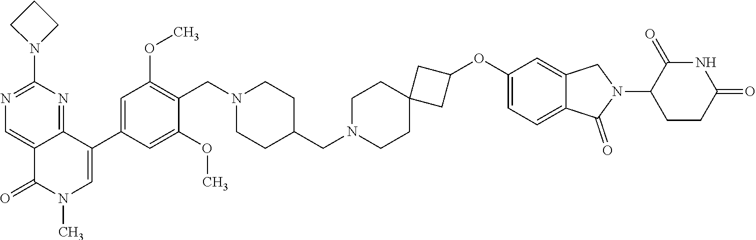 [SMILES: COc1cc(-c2cn(C)c(=O)c3cnc(N4CCC4)nc23)cc(OC)c1CN1CCC(CN2CCC3(CC2)CC(Oc2ccc4c(c2)CN(C2CCC(=O)NC2=O)C4=O)C3)CC1] |
| H183 | 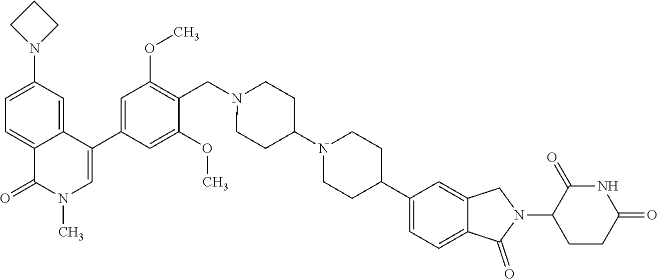 [SMILES: COc1cc(-c2cn(C)c(=O)c3ccc(N4CCC4)cc23)cc(OC)c1CN1CCC(N2CCC(c3ccc4c(c3)CN(C3CCC(=O)NC3=O)C4=O)CC2)CC1] |
| H184 | 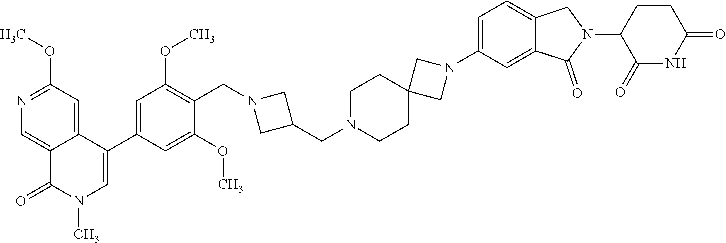 [SMILES: COc1cc2c(-c3cc(OC)c(CN4CC(CN5CCC6(CC5)CN(c5ccc7c(c5)C(=O)N(C5CCC(=O)NC5=O)C7)C6)C4)c(OC)c3)cn(C)c(=O)c2cn1] |
| H185 | 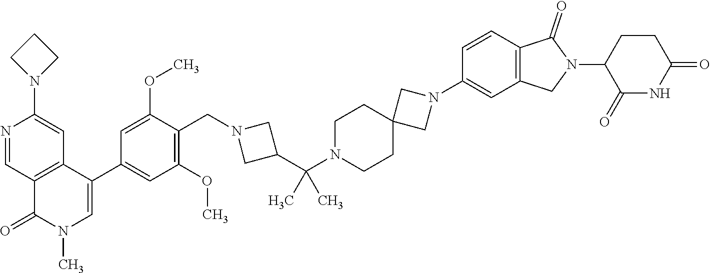 [SMILES: COc1cc(-c2cn(C)c(=O)c3cnc(N4CCC4)cc23)cc(OC)c1CN1CC(C(C)(C)N2CCC3(CC2)CN(c2ccc4c(c2)CN(C2CCC(=O)NC2=O)C4=O)C3)C1] |
| H186 | 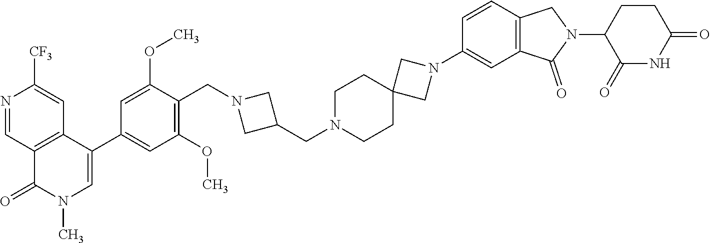 [SMILES: COc1cc(-c2cn(C)c(=O)c3cnc(C(F)(F)F)cc23)cc(OC)c1CN1CC(CN2CCC3(CC2)CN(c2ccc4c(c2)C(=O)N(C2CCC(=O)NC2=O)C4)C3)C1] |
| H187 | 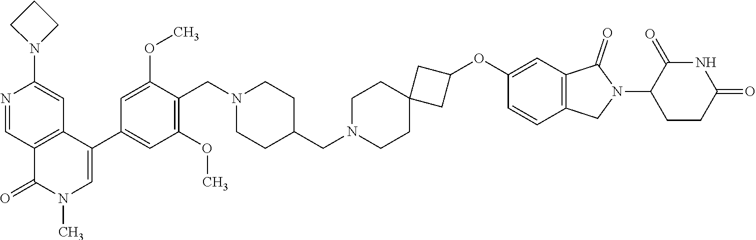 [SMILES: COc1cc(-c2cn(C)c(=O)c3cnc(N4CCC4)cc23)cc(OC)c1CN1CCC(CN2CCC3(CC2)CC(Oc2ccc4c(c2)C(=O)N(C2CCC(=O)NC2=O)C4)C3)CC1] |
| H188 | 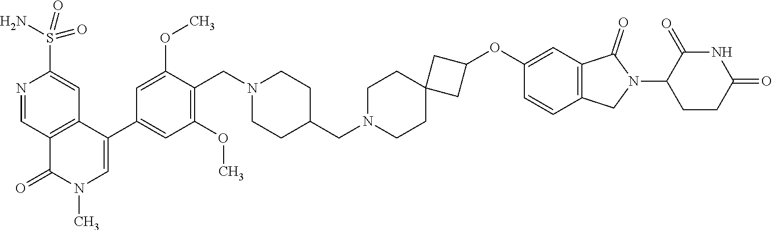 [SMILES: COc1cc(-c2cn(C)c(=O)c3cnc(S(N)(=O)=O)cc23)cc(OC)c1CN1CCC(CN2CCC3(CC2)CC(Oc2ccc4c(c2)C(=O)N(C2CCC(=O)NC2=O)C4)C3)CC1] |
| H189 | 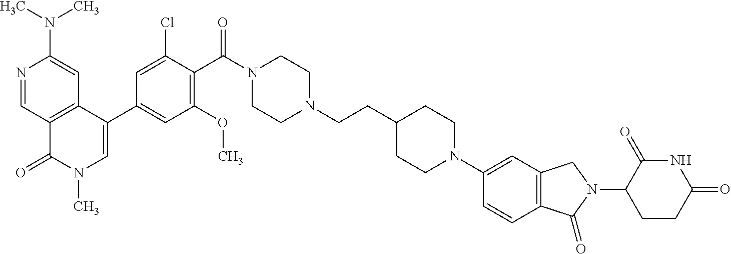 [SMILES: COc1cc(-c2cn(C)c(=O)c3cnc(N(C)C)cc23)cc(Cl)c1C(=O)N1CCN(CCC2CCN(c3ccc4c(c3)CN(C3CCC(=O)NC3=O)C4=O)CC2)CC1] |
| H190 | 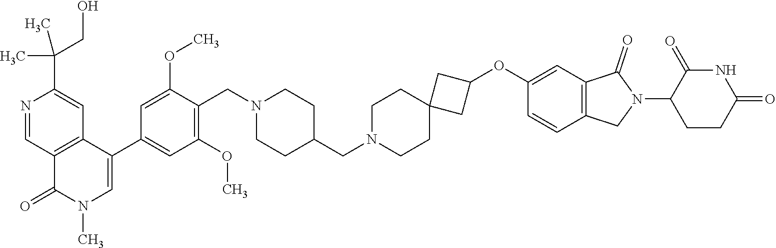 [SMILES: COc1cc(-c2cn(C)c(=O)c3cnc(C(C)(C)CO)cc23)cc(OC)c1CN1CCC(CN2CCC3(CC2)CC(Oc2ccc4c(c2)C(=O)N(C2CCC(=O)NC2=O)C4)C3)CC1] |
| H191 | 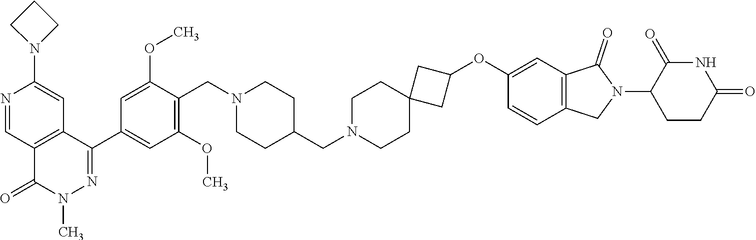 [SMILES: COc1cc(-c2nn(C)c(=O)c3cnc(N4CCC4)cc23)cc(OC)c1CN1CCC(CN2CCC3(CC2)CC(Oc2ccc4c(c2)C(=O)N(C2CCC(=O)NC2=O)C4)C3)CC1] |
| H192 | 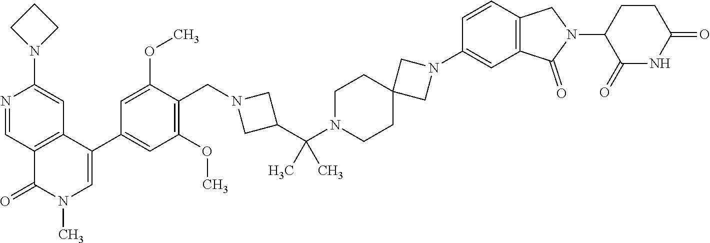 [SMILES: COc1cc(-c2cn(C)c(=O)c3cnc(N4CCC4)cc23)cc(OC)c1CN1CC(C(C)(C)N2CCC3(CC2)CN(c2ccc4c(c2)C(=O)N(C2CCC(=O)NC2=O)C4)C3)C1] |
| H193 | 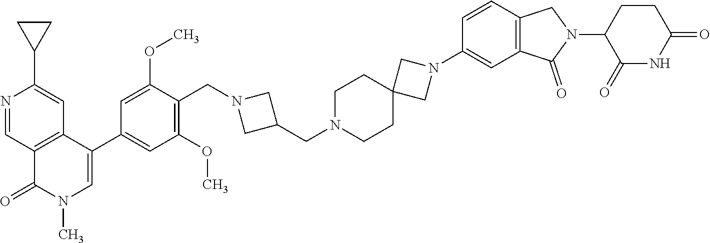 [SMILES: COc1cc(-c2cn(C)c(=O)c3cnc(C4CC4)cc23)cc(OC)c1CN1CC(CN2CCC3(CC2)CN(c2ccc4c(c2)C(=O)N(C2CCC(=O)NC2=O)C4)C3)C1] |
| H194 | 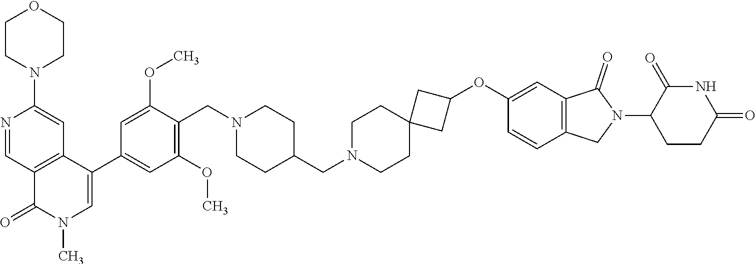 [SMILES: COc1cc(-c2cn(C)c(=O)c3cnc(N4CCOCC4)cc23)cc(OC)c1CN1CCC(CN2CCC3(CC2)CC(Oc2ccc4c(c2)C(=O)N(C2CCC(=O)NC2=O)C4)C3)CC1] |
| H195 | 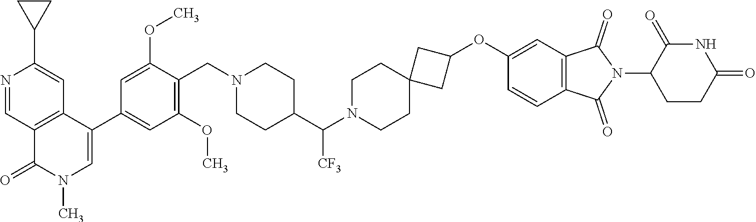 [SMILES: COc1cc(-c2cn(C)c(=O)c3cnc(C4CC4)cc23)cc(OC)c1CN1CCC(C(N2CCC3(CC2)CC(Oc2ccc4c(c2)C(=O)N(C2CCC(=O)NC2=O)C4=O)C3)C(F)(F)F)CC1] |
| H196 | 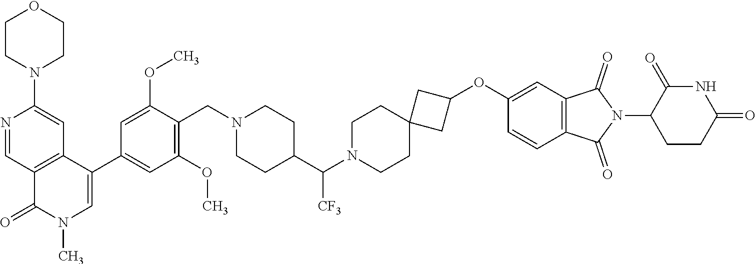 [SMILES: COc1cc(-c2cn(C)c(=O)c3cnc(N4CCOCC4)cc23)cc(OC)c1CN1CCC(C(N2CCC3(CC2)CC(Oc2ccc4c(c2)C(=O)N(C2CCC(=O)NC2=O)C4=O)C3)C(F)(F)F)CC1] |
| H197 | 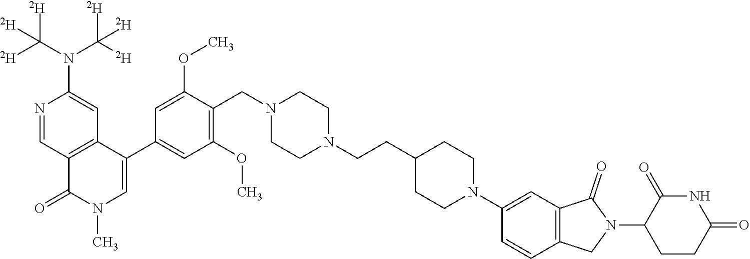 [SMILES: [2H]C([2H])([2H])N(c1cc2c(-c3cc(OC)c(CN4CCN(CCC5CCN(c6ccc7c(c6)C(=O)N(C6CCC(=O)NC6=O)C7)CC5)CC4)c(OC)c3)cn(C)c(=O)c2cn1)C([2H])([2H])[2H]] |
| H198 | 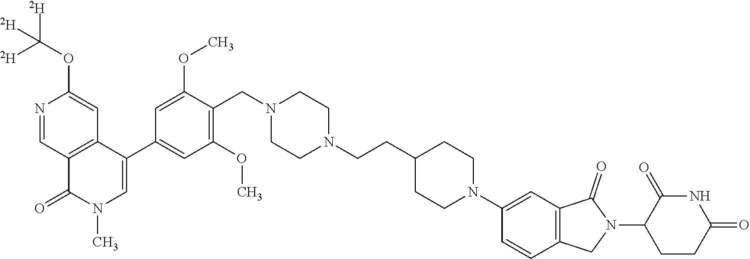 [SMILES: [2H]C([2H])([2H])Oc1cc2c(-c3cc(OC)c(CN4CCN(CCC5CCN(c6ccc7c(c6)C(=O)N(C6CCC(=O)NC6=O)C7)CC5)CC4)c(OC)c3)cn(C)c(=O)c2cn1] |
| H199 | 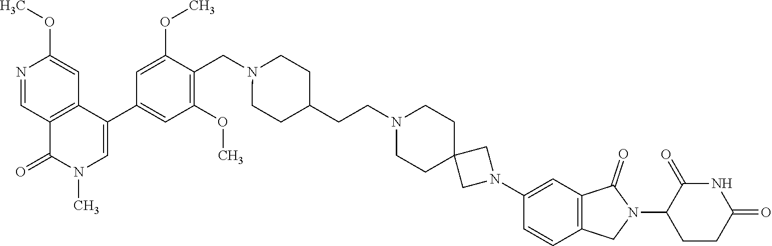 [SMILES: COc1cc2c(-c3cc(OC)c(CN4CCC(CCN5CCC6(CC5)CN(c5ccc7c(c5)C(=O)N(C5CCC(=O)NC5=O)C7)C6)CC4)c(OC)c3)cn(C)c(=O)c2cn1] |
| H200 | 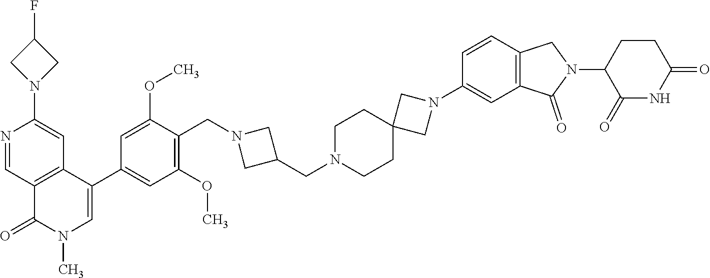 [SMILES: COc1cc(-c2cn(C)c(=O)c3cnc(N4CC(F)C4)cc23)cc(OC)c1CN1CC(CN2CCC3(CC2)CN(c2ccc4c(c2)C(=O)N(C2CCC(=O)NC2=O)C4)C3)C1] |
| H201 | 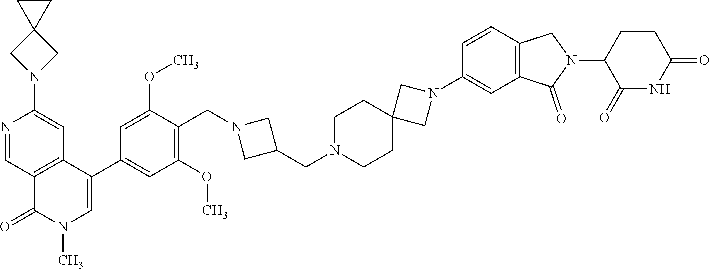 [SMILES: COc1cc(-c2cn(C)c(=O)c3cnc(N4CC5(CC5)C4)cc23)cc(OC)c1CN1CC(CN2CCC3(CC2)CN(c2ccc4c(c2)C(=O)N(C2CCC(=O)NC2=O)C4)C3)C1] |
| H202 | 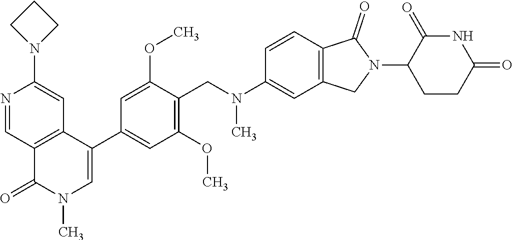 [SMILES: COc1cc(-c2cn(C)c(=O)c3cnc(N4CCC4)cc23)cc(OC)c1CN(C)c1ccc2c(c1)CN(C1CCC(=O)NC1=O)C2=O] |
| H203 | 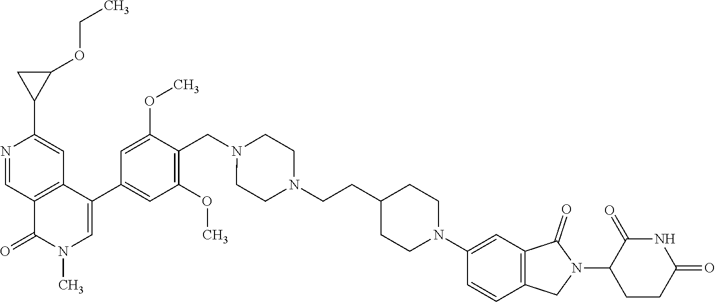 [SMILES: CCOC1CC1c1cc2c(-c3cc(OC)c(CN4CCN(CCC5CCN(c6ccc7c(c6)C(=O)N(C6CCC(=O)NC6=O)C7)CC5)CC4)c(OC)c3)cn(C)c(=O)c2cn1] |
| H204 | 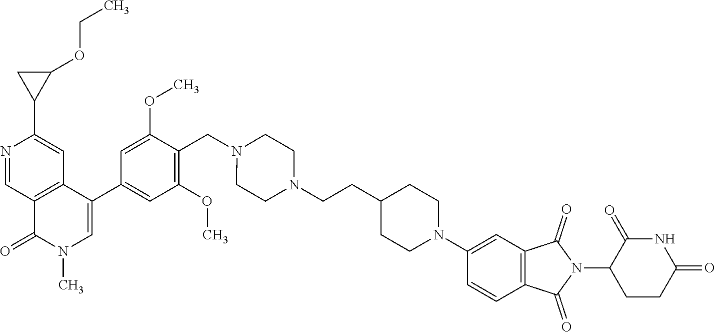 [SMILES: CCOC1CC1c1cc2c(-c3cc(OC)c(CN4CCN(CCC5CCN(c6ccc7c(c6)C(=O)N(C6CCC(=O)NC6=O)C7=O)CC5)CC4)c(OC)c3)cn(C)c(=O)c2cn1] |
| H205 | 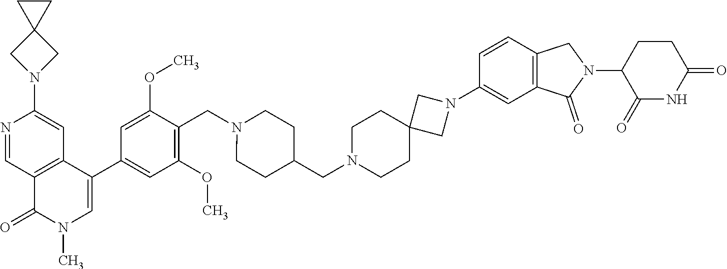 [SMILES: COc1cc(-c2cn(C)c(=O)c3cnc(N4CC5(CC5)C4)cc23)cc(OC)c1CN1CCC(CN2CCC3(CC2)CN(c2ccc4c(c2)C(=O)N(C2CCC(=O)NC2=O)C4)C3)CC1] |
| H206 | 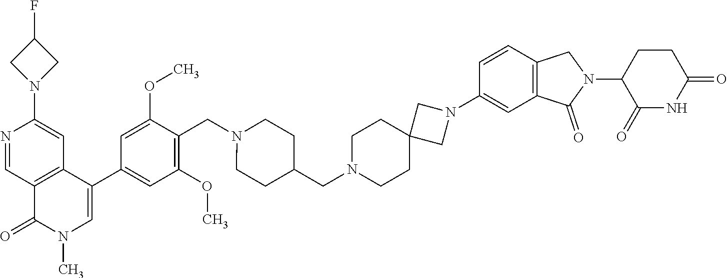 [SMILES: COc1cc(-c2cn(C)c(=O)c3cnc(N4CC(F)C4)cc23)cc(OC)c1CN1CCC(CN2CCC3(CC2)CN(c2ccc4c(c2)C(=O)N(C2CCC(=O)NC2=O)C4)C3)CC1] |
| H207 | 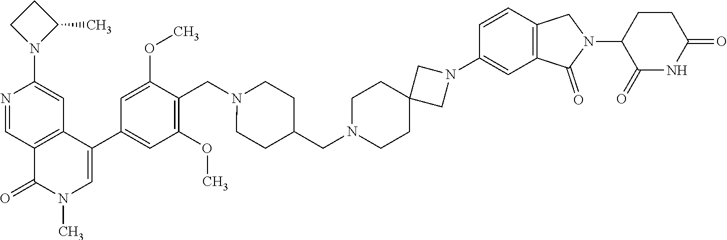 [SMILES: COc1cc(-c2cn(C)c(=O)c3cnc(N4CC[C@@H]4C)cc23)cc(OC)c1CN1CCC(CN2CCC3(CC2)CN(c2ccc4c(c2)C(=O)N(C2CCC(=O)NC2=O)C4)C3)CC1] |
| H208 | 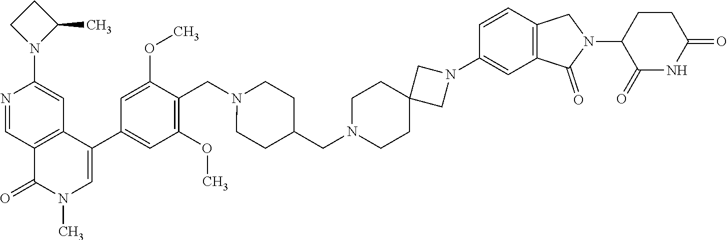 [SMILES: COc1cc(-c2cn(C)c(=O)c3cnc(N4CC[C@H]4C)cc23)cc(OC)c1CN1CCC(CN2CCC3(CC2)CN(c2ccc4c(c2)C(=O)N(C2CCC(=O)NC2=O)C4)C3)CC1] |
| H209 | 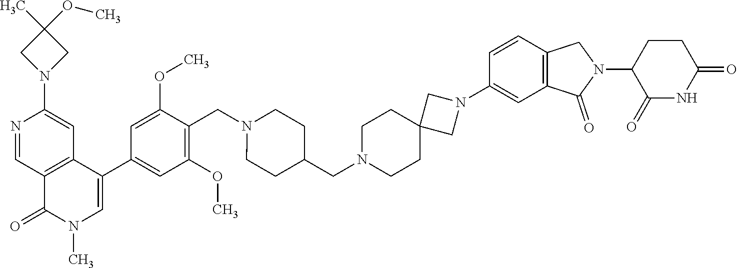 [SMILES: COc1cc(-c2cn(C)c(=O)c3cnc(N4CC(C)(OC)C4)cc23)cc(OC)c1CN1CCC(CN2CCC3(CC2)CN(c2ccc4c(c2)C(=O)N(C2CCC(=O)NC2=O)C4)C3)CC1] |
| H210 | 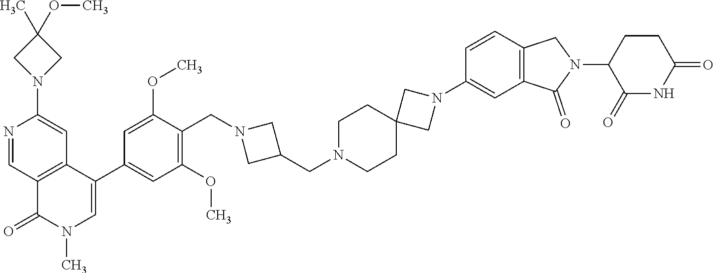 [SMILES: COc1cc(-c2cn(C)c(=O)c3cnc(N4CC(C)(OC)C4)cc23)cc(OC)c1CN1CC(CN2CCC3(CC2)CN(c2ccc4c(c2)C(=O)N(C2CCC(=O)NC2=O)C4)C3)C1] |
| H211 | 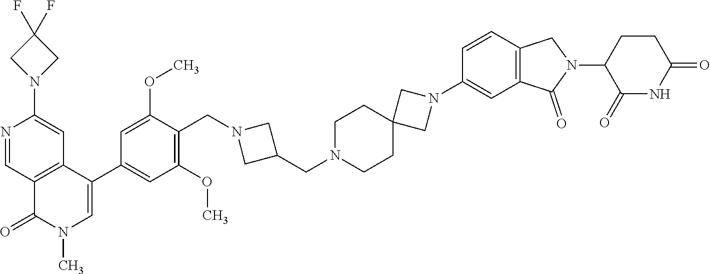 [SMILES: COc1cc(-c2cn(C)c(=O)c3cnc(N4CC(F)(F)C4)cc23)cc(OC)c1CN1CC(CN2CCC3(CC2)CN(c2ccc4c(c2)C(=O)N(C2CCC(=O)NC2=O)C4)C3)C1] |
| H212 | 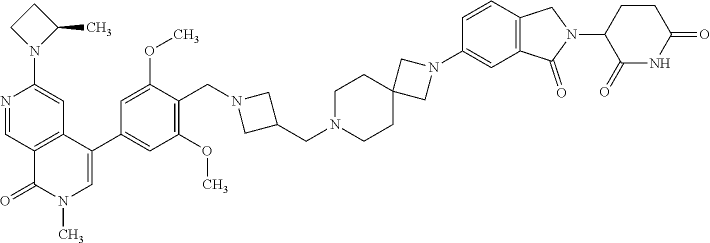 [SMILES: COc1cc(-c2cn(C)c(=O)c3cnc(N4CC[C@H]4C)cc23)cc(OC)c1CN1CC(CN2CCC3(CC2)CN(c2ccc4c(c2)C(=O)N(C2CCC(=O)NC2=O)C4)C3)C1] |
| H213 | 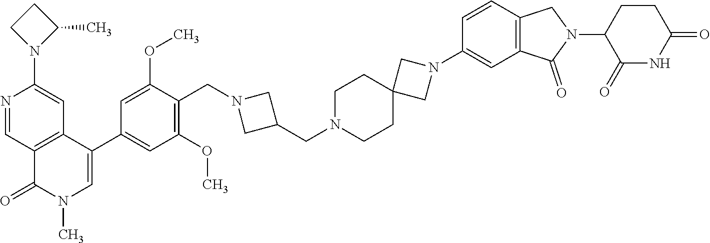 [SMILES: COc1cc(-c2cn(C)c(=O)c3cnc(N4CC[C@@H]4C)cc23)cc(OC)c1CN1CC(CN2CCC3(CC2)CN(c2ccc4c(c2)C(=O)N(C2CCC(=O)NC2=O)C4)C3)C1] |
| H214 | 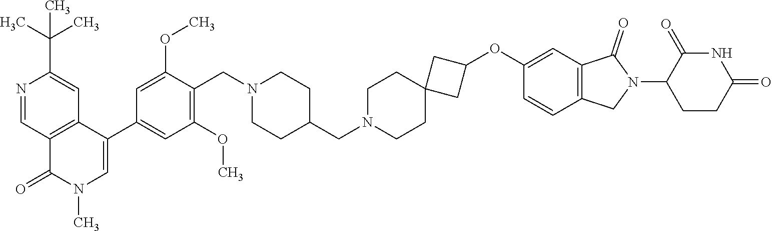 [SMILES: COc1cc(-c2cn(C)c(=O)c3cnc(C(C)(C)C)cc23)cc(OC)c1CN1CCC(CN2CCC3(CC2)CC(Oc2ccc4c(c2)C(=O)N(C2CCC(=O)NC2=O)C4)C3)CC1] |
| H215 | 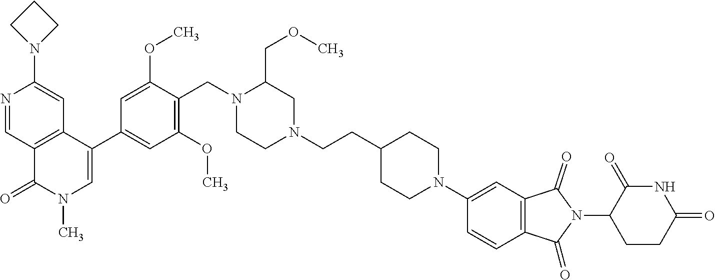 [SMILES: COCC1CN(CCC2CCN(c3ccc4c(c3)C(=O)N(C3CCC(=O)NC3=O)C4=O)CC2)CCN1Cc1c(OC)cc(-c2cn(C)c(=O)c3cnc(N4CCC4)cc23)cc1OC] |
| H216 | 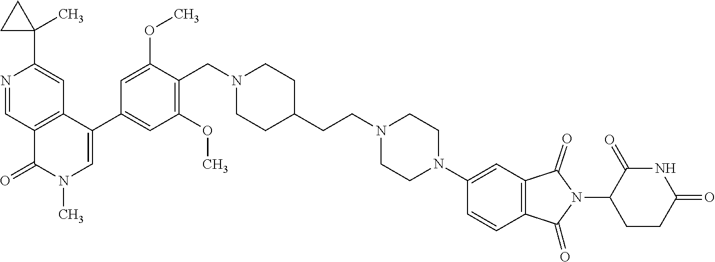 [SMILES: COc1cc(-c2cn(C)c(=O)c3cnc(C4(C)CC4)cc23)cc(OC)c1CN1CCC(CCN2CCN(c3ccc4c(c3)C(=O)N(C3CCC(=O)NC3=O)C4=O)CC2)CC1] |
| H217 | 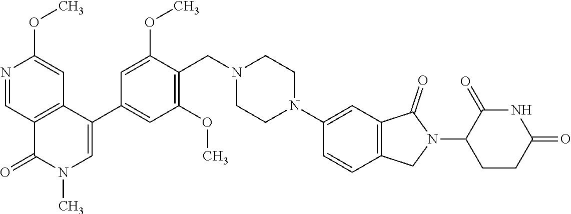 [SMILES: COc1cc2c(-c3cc(OC)c(CN4CCN(c5ccc6c(c5)C(=O)N(C5CCC(=O)NC5=O)C6)CC4)c(OC)c3)cn(C)c(=O)c2cn1] |
| H218 | 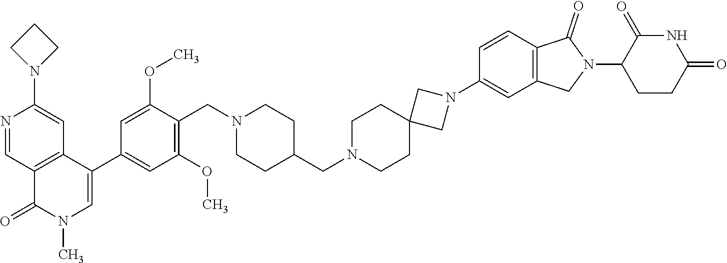 [SMILES: COc1cc(-c2cn(C)c(=O)c3cnc(N4CCC4)cc23)cc(OC)c1CN1CCC(CN2CCC3(CC2)CN(c2ccc4c(c2)CN(C2CCC(=O)NC2=O)C4=O)C3)CC1] |
| H219 | 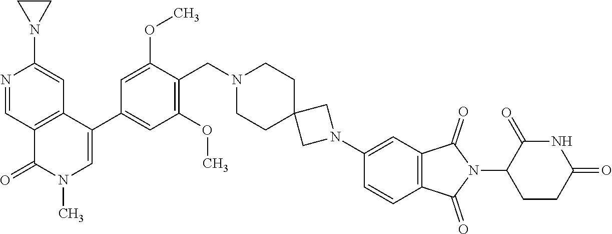 [SMILES: COc1cc(-c2cn(C)c(=O)c3cnc(N4CC4)cc23)cc(OC)c1CN1CCC2(CC1)CN(c1ccc3c(c1)C(=O)N(C1CCC(=O)NC1=O)C3=O)C2] |
| H220 | 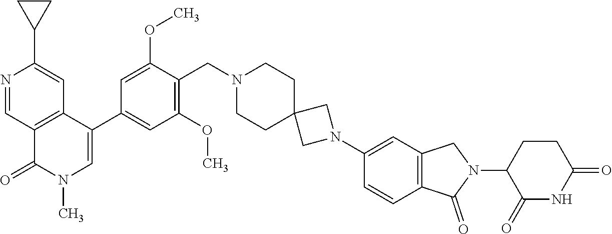 [SMILES: COc1cc(-c2cn(C)c(=O)c3cnc(C4CC4)cc23)cc(OC)c1CN1CCC2(CC1)CN(c1ccc3c(c1)CN(C1CCC(=O)NC1=O)C3=O)C2] |
| H221 | 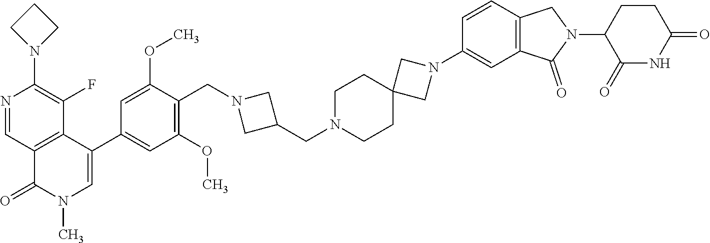 [SMILES: COc1cc(-c2cn(C)c(=O)c3cnc(N4CCC4)c(F)c23)cc(OC)c1CN1CC(CN2CCC3(CC2)CN(c2ccc4c(c2)C(=O)N(C2CCC(=O)NC2=O)C4)C3)C1] |
| H222 | 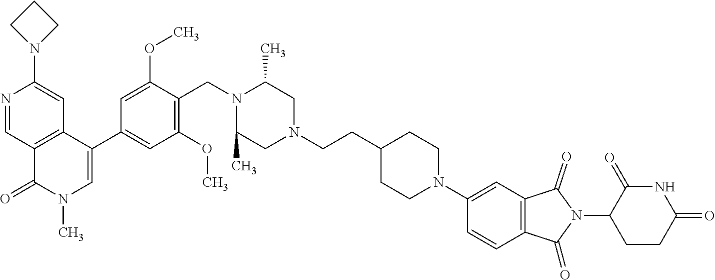 [SMILES: COc1cc(-c2cn(C)c(=O)c3cnc(N4CCC4)cc23)cc(OC)c1CN1[C@H](C)CN(CCC2CCN(c3ccc4c(c3)C(=O)N(C3CCC(=O)NC3=O)C4=O)CC2)C[C@H]1C] |
| H223 | 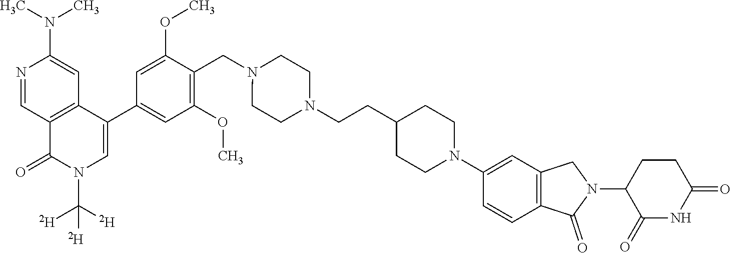 [SMILES: [2H]C([2H])([2H])n1cc(-c2cc(OC)c(CN3CCN(CCC4CCN(c5ccc6c(c5)CN(C5CCC(=O)NC5=O)C6=O)CC4)CC3)c(OC)c2)c2cc(N(C)C)ncc2c1=O] |
| H224 | 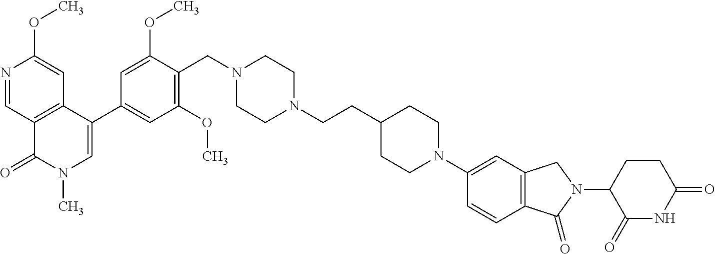 [SMILES: COc1cc2c(-c3cc(OC)c(CN4CCN(CCC5CCN(c6ccc7c(c6)CN(C6CCC(=O)NC6=O)C7=O)CC5)CC4)c(OC)c3)cn(C)c(=O)c2cn1] |
| H225 | 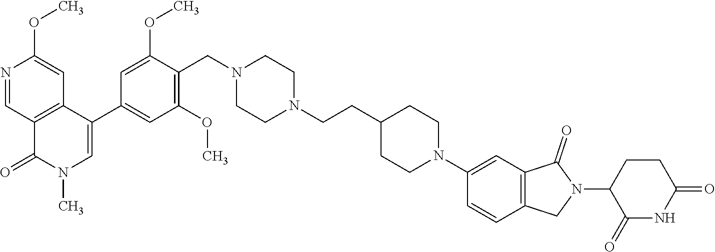 [SMILES: COc1cc2c(-c3cc(OC)c(CN4CCN(CCC5CCN(c6ccc7c(c6)C(=O)N(C6CCC(=O)NC6=O)C7)CC5)CC4)c(OC)c3)cn(C)c(=O)c2cn1] |
| H226 | 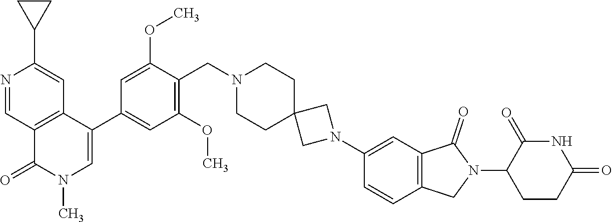 [SMILES: COc1cc(-c2cn(C)c(=O)c3cnc(C4CC4)cc23)cc(OC)c1CN1CCC2(CC1)CN(c1ccc3c(c1)C(=O)N(C1CCC(=O)NC1=O)C3)C2] |
| H227 | 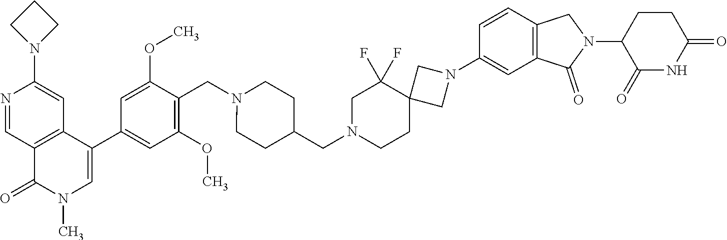 [SMILES: COc1cc(-c2cn(C)c(=O)c3cnc(N4CCC4)cc23)cc(OC)c1CN1CCC(CN2CCC3(CN(c4ccc5c(c4)C(=O)N(C4CCC(=O)NC4=O)C5)C3)C(F)(F)C2)CC1] |
| H228 | 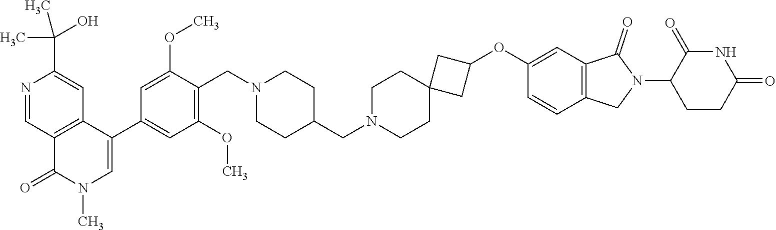 [SMILES: COc1cc(-c2cn(C)c(=O)c3cnc(C(C)(C)O)cc23)cc(OC)c1CN1CCC(CN2CCC3(CC2)CC(Oc2ccc4c(c2)C(=O)N(C2CCC(=O)NC2=O)C4)C3)CC1] |
| H229 | 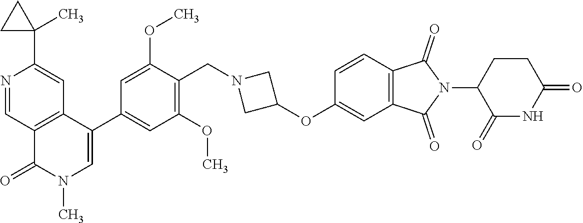 [SMILES: COc1cc(-c2cn(C)c(=O)c3cnc(C4(C)CC4)cc23)cc(OC)c1CN1CC(Oc2ccc3c(c2)C(=O)N(C2CCC(=O)NC2=O)C3=O)C1] |
| H230 | 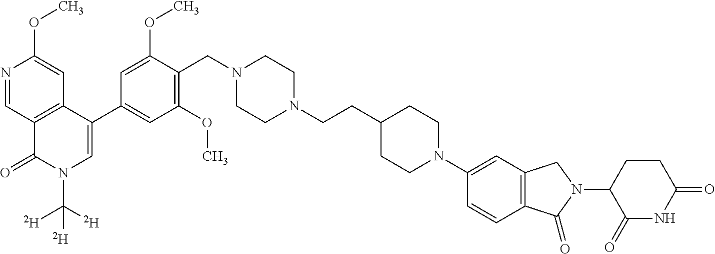 [SMILES: [2H]C([2H])([2H])n1cc(-c2cc(OC)c(CN3CCN(CCC4CCN(c5ccc6c(c5)CN(C5CCC(=O)NC5=O)C6=O)CC4)CC3)c(OC)c2)c2cc(OC)ncc2c1=O] |
| H231 | 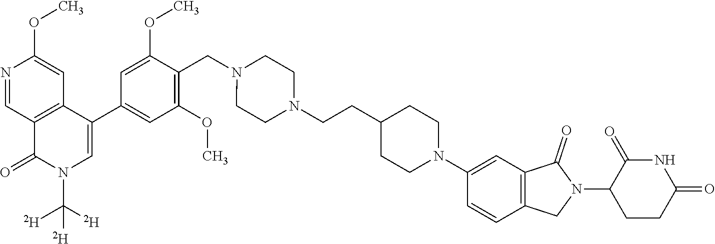 [SMILES: [2H]C([2H])([2H])n1cc(-c2cc(OC)c(CN3CCN(CCC4CCN(c5ccc6c(c5)C(=O)N(C5CCC(=O)NC5=O)C6)CC4)CC3)c(OC)c2)c2cc(OC)ncc2c1=O] |
| H232 | 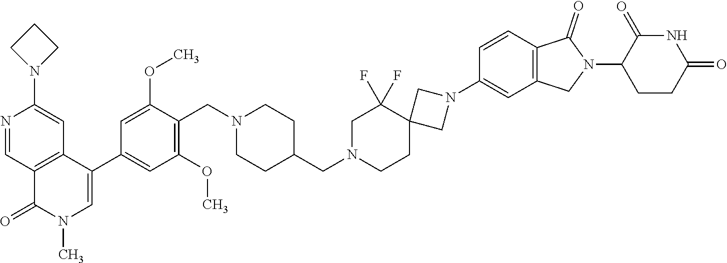 [SMILES: COc1cc(-c2cn(C)c(=O)c3cnc(N4CCC4)cc23)cc(OC)c1CN1CCC(CN2CCC3(CN(c4ccc5c(c4)CN(C4CCC(=O)NC4=O)C5=O)C3)C(F)(F)C2)CC1] |
| H233 | 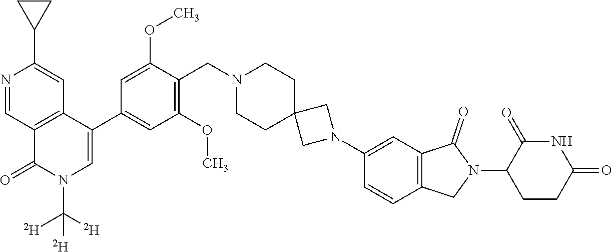 [SMILES: [2H]C([2H])([2H])n1cc(-c2cc(OC)c(CN3CCC4(CC3)CN(c3ccc5c(c3)C(=O)N(C3CCC(=O)NC3=O)C5)C4)c(OC)c2)c2cc(C3CC3)ncc2c1=O] |
| H234 | 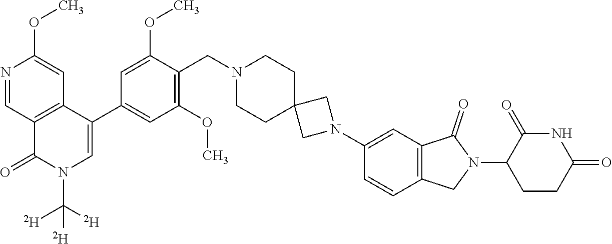 [SMILES: [2H]C([2H])([2H])n1cc(-c2cc(OC)c(CN3CCC4(CC3)CN(c3ccc5c(c3)C(=O)N(C3CCC(=O)NC3=O)C5)C4)c(OC)c2)c2cc(OC)ncc2c1=O] |
| H235 | 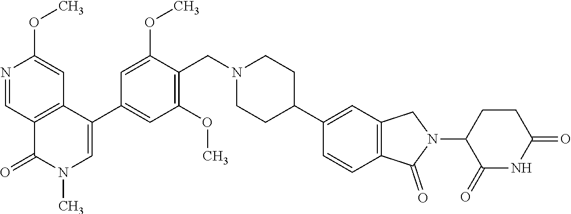 [SMILES: COc1cc2c(-c3cc(OC)c(CN4CCC(c5ccc6c(c5)CN(C5CCC(=O)NC5=O)C6=O)CC4)c(OC)c3)cn(C)c(=O)c2cn1] |
| H236 | 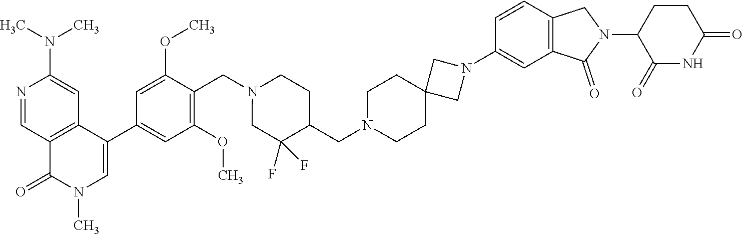 [SMILES: COc1cc(-c2cn(C)c(=O)c3cnc(N(C)C)cc23)cc(OC)c1CN1CCC(CN2CCC3(CC2)CN(c2ccc4c(c2)C(=O)N(C2CCC(=O)NC2=O)C4)C3)C(F)(F)C1] |
| H237 | 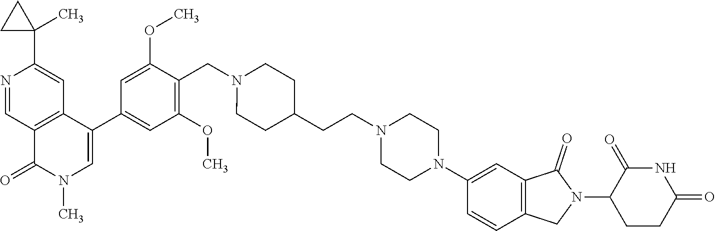 [SMILES: COc1cc(-c2cn(C)c(=O)c3cnc(C4(C)CC4)cc23)cc(OC)c1CN1CCC(CCN2CCN(c3ccc4c(c3)C(=O)N(C3CCC(=O)NC3=O)C4)CC2)CC1] |
| H238 | 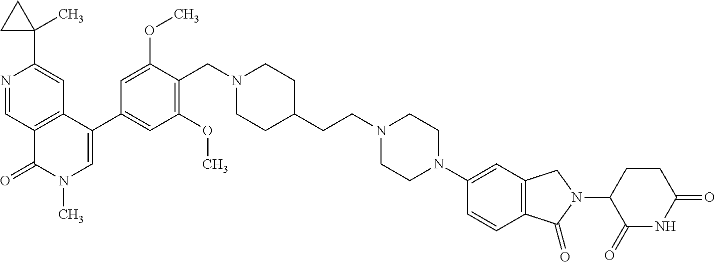 [SMILES: COc1cc(-c2cn(C)c(=O)c3cnc(C4(C)CC4)cc23)cc(OC)c1CN1CCC(CCN2CCN(c3ccc4c(c3)CN(C3CCC(=O)NC3=O)C4=O)CC2)CC1] |
| H239 | 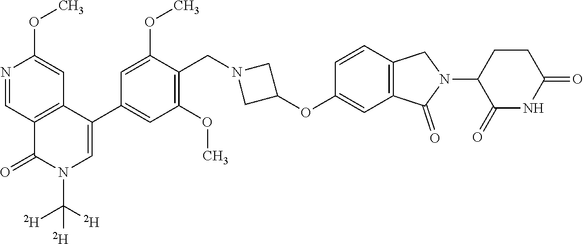 [SMILES: [2H]C([2H])([2H])n1cc(-c2cc(OC)c(CN3CC(Oc4ccc5c(c4)C(=O)N(C4CCC(=O)NC4=O)C5)C3)c(OC)c2)c2cc(OC)ncc2c1=O] |
| H240 | 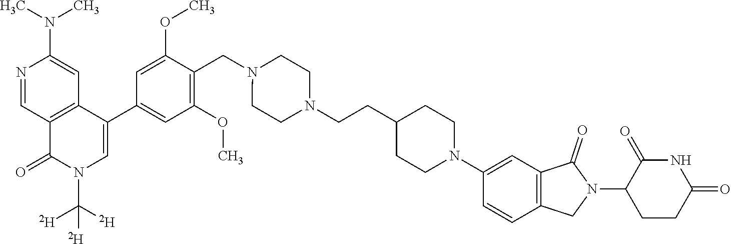 [SMILES: [2H]C([2H])([2H])n1cc(-c2cc(OC)c(CN3CCN(CCC4CCN(c5ccc6c(c5)C(=O)N(C5CCC(=O)NC5=O)C6)CC4)CC3)c(OC)c2)c2cc(N(C)C)ncc2c1=O] |
| H241 | 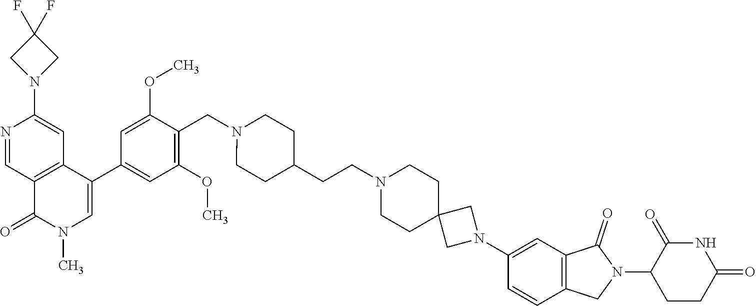 [SMILES: COc1cc(-c2cn(C)c(=O)c3cnc(N4CC(F)(F)C4)cc23)cc(OC)c1CN1CCC(CCN2CCC3(CC2)CN(c2ccc4c(c2)C(=O)N(C2CCC(=O)NC2=O)C4)C3)CC1] |
| H242 | 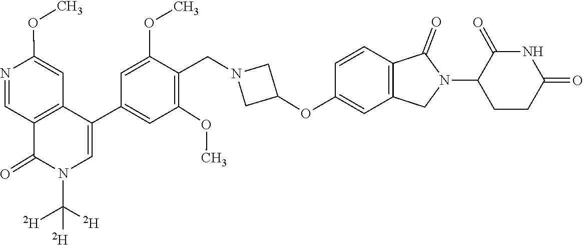 [SMILES: [2H]C([2H])([2H])n1cc(-c2cc(OC)c(CN3CC(Oc4ccc5c(c4)CN(C4CCC(=O)NC4=O)C5=O)C3)c(OC)c2)c2cc(OC)ncc2c1=O] |
| H243 | 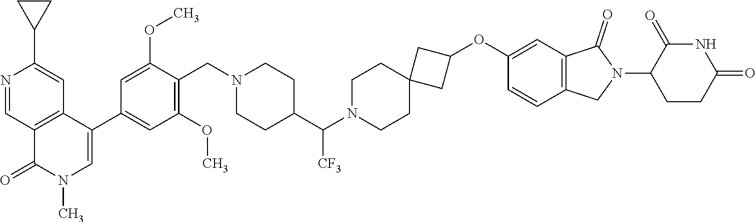 [SMILES: COc1cc(-c2cn(C)c(=O)c3cnc(C4CC4)cc23)cc(OC)c1CN1CCC(C(N2CCC3(CC2)CC(Oc2ccc4c(c2)C(=O)N(C2CCC(=O)NC2=O)C4)C3)C(F)(F)F)CC1] |
| H244 | 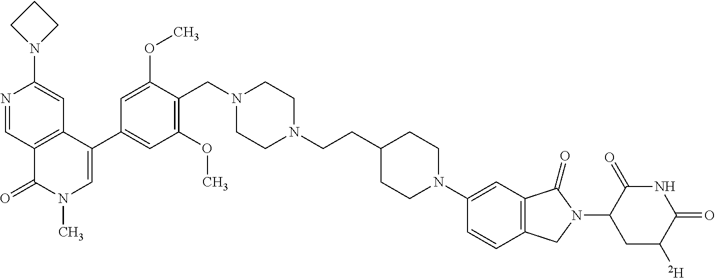 [SMILES: [2H]C1CC(N2Cc3ccc(N4CCC(CCN5CCN(Cc6c(OC)cc(-c7cn(C)c(=O)c8cnc(N9CCC9)cc78)cc6OC)CC5)CC4)cc3C2=O)C(=O)NC1=O] |
| H245 | 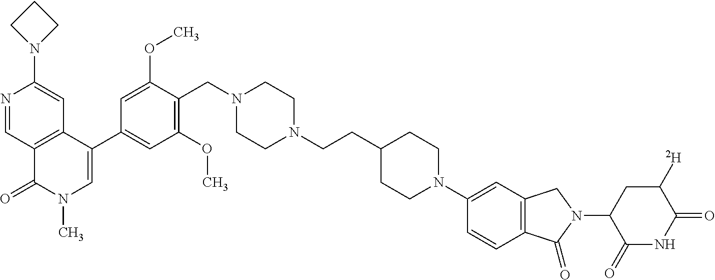 [SMILES: [2H]C1CC(N2Cc3cc(N4CCC(CCN5CCN(Cc6c(OC)cc(-c7cn(C)c(=O)c8cnc(N9CCC9)cc78)cc6OC)CC5)CC4)ccc3C2=O)C(=O)NC1=O] |
| H246 | 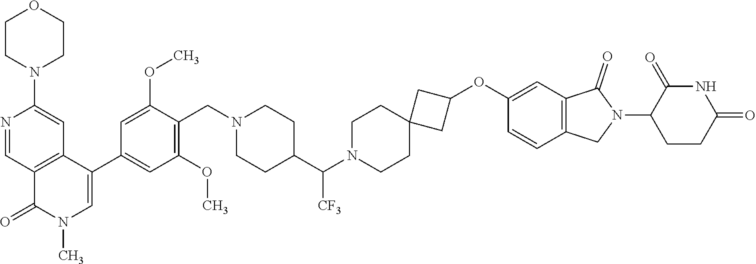 [SMILES: COc1cc(-c2cn(C)c(=O)c3cnc(N4CCOCC4)cc23)cc(OC)c1CN1CCC(C(N2CCC3(CC2)CC(Oc2ccc4c(c2)C(=O)N(C2CCC(=O)NC2=O)C4)C3)C(F)(F)F)CC1] |
| H247 | 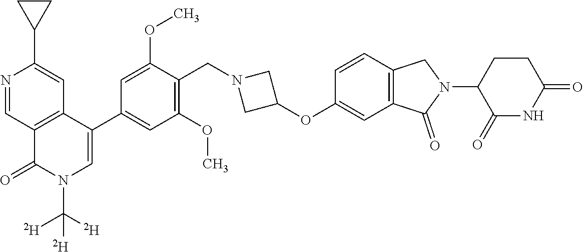 [SMILES: [2H]C([2H])([2H])n1cc(-c2cc(OC)c(CN3CC(Oc4ccc5c(c4)C(=O)N(C4CCC(=O)NC4=O)C5)C3)c(OC)c2)c2cc(C3CC3)ncc2c1=O] |
| H248 | 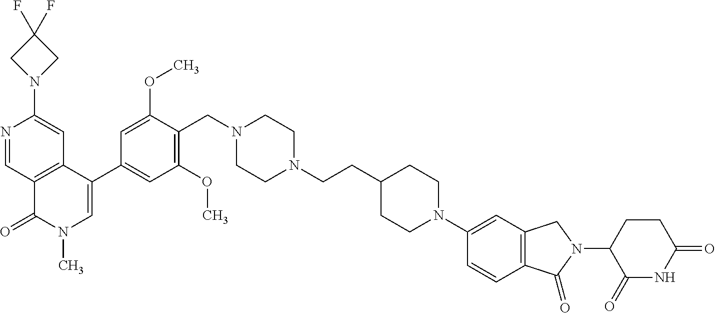 [SMILES: COc1cc(-c2cn(C)c(=O)c3cnc(N4CC(F)(F)C4)cc23)cc(OC)c1CN1CCN(CCC2CCN(c3ccc4c(c3)CN(C3CCC(=O)NC3=O)C4=O)CC2)CC1] |
| H249 | 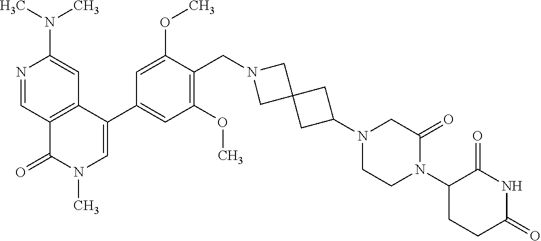 [SMILES: COc1cc(-c2cn(C)c(=O)c3cnc(N(C)C)cc23)cc(OC)c1CN1CC2(CC(N3CCN(C4CCC(=O)NC4=O)C(=O)C3)C2)C1] |
| H250 | 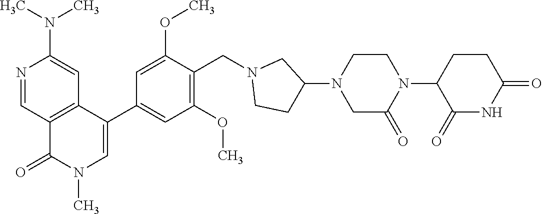 [SMILES: COc1cc(-c2cn(C)c(=O)c3cnc(N(C)C)cc23)cc(OC)c1CN1CCC(N2CCN(C3CCC(=O)NC3=O)C(=O)C2)C1] |
| H251 | 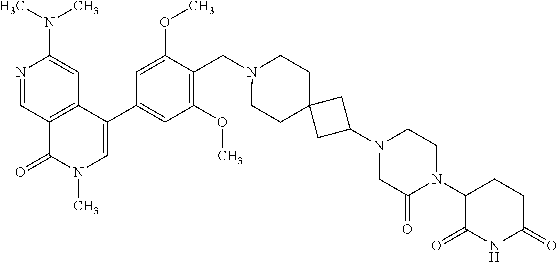 [SMILES: COc1cc(-c2cn(C)c(=O)c3cnc(N(C)C)cc23)cc(OC)c1CN1CCC2(CC1)CC(N1CCN(C3CCC(=O)NC3=O)C(=O)C1)C2] |
| H252 | 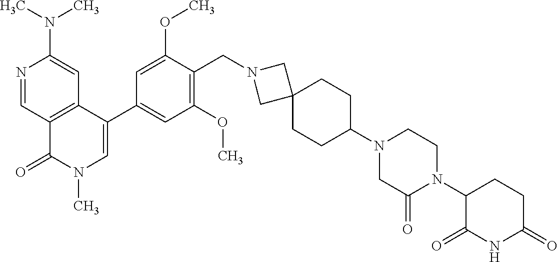 [SMILES: COc1cc(-c2cn(C)c(=O)c3cnc(N(C)C)cc23)cc(OC)c1CN1CC2(CCC(N3CCN(C4CCC(=O)NC4=O)C(=O)C3)CC2)C1] |
| H253 | 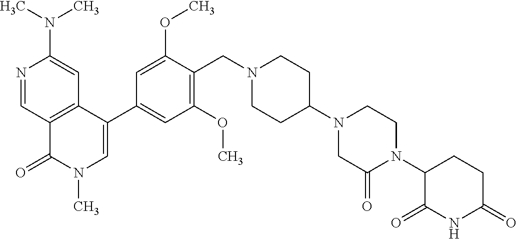 [SMILES: COc1cc(-c2cn(C)c(=O)c3cnc(N(C)C)cc23)cc(OC)c1CN1CCC(N2CCN(C3CCC(=O)NC3=O)C(=O)C2)CC1] |
| H254 | 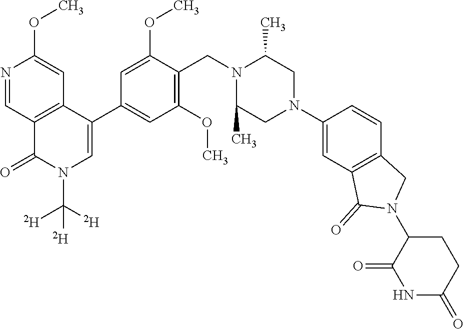 [SMILES: [2H]C([2H])([2H])n1cc(-c2cc(OC)c(CN3[C@H](C)CN(c4ccc5c(c4)C(=O)N(C4CCC(=O)NC4=O)C5)C[C@H]3C)c(OC)c2)c2cc(OC)ncc2c1=O] |
| H255 | 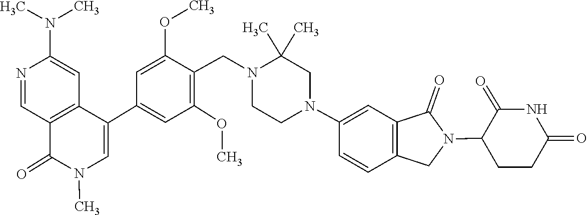 [SMILES: COc1cc(-c2cn(C)c(=O)c3cnc(N(C)C)cc23)cc(OC)c1CN1CCN(c2ccc3c(c2)C(=O)N(C2CCC(=O)NC2=O)C3)CC1(C)C] |
| H256 | 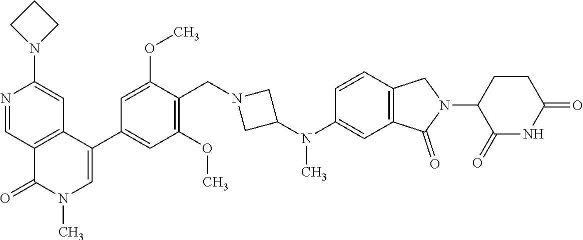 [SMILES: COc1cc(-c2cn(C)c(=O)c3cnc(N4CCC4)cc23)cc(OC)c1CN1CC(N(C)c2ccc3c(c2)C(=O)N(C2CCC(=O)NC2=O)C3)C1] |
| H257 | 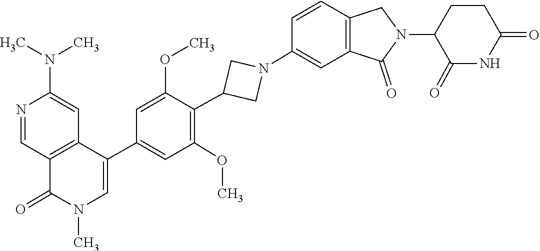 [SMILES: COc1cc(-c2cn(C)c(=O)c3cnc(N(C)C)cc23)cc(OC)c1C1CN(c2ccc3c(c2)C(=O)N(C2CCC(=O)NC2=O)C3)C1] |
| H258 | 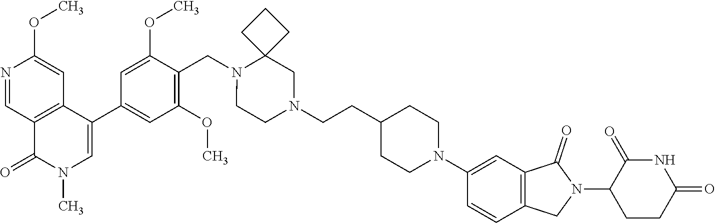 [SMILES: COc1cc2c(-c3cc(OC)c(CN4CCN(CCC5CCN(c6ccc7c(c6)C(=O)N(C6CCC(=O)NC6=O)C7)CC5)CC45CCC5)c(OC)c3)cn(C)c(=O)c2cn1] |
| H259 | 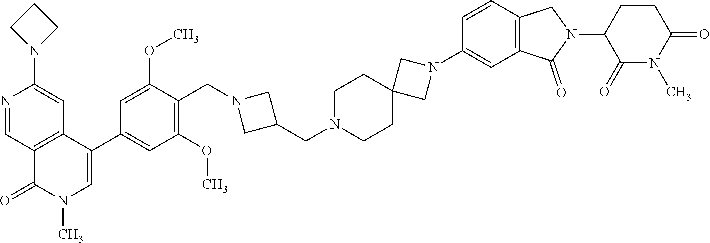 [SMILES: COc1cc(-c2cn(C)c(=O)c3cnc(N4CCC4)cc23)cc(OC)c1CN1CC(CN2CCC3(CC2)CN(c2ccc4c(c2)C(=O)N(C2CCC(=O)N(C)C2=O)C4)C3)C1] |
| H260 | 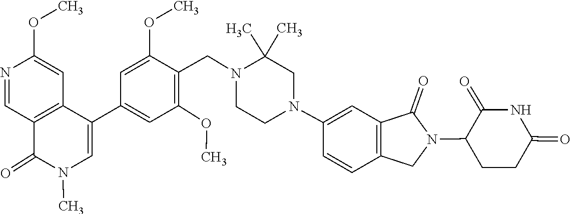 [SMILES: COc1cc2c(-c3cc(OC)c(CN4CCN(c5ccc6c(c5)C(=O)N(C5CCC(=O)NC5=O)C6)CC4(C)C)c(OC)c3)cn(C)c(=O)c2cn1] |
| H261 | 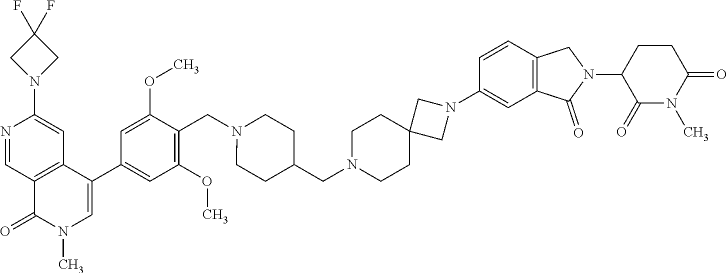 [SMILES: COc1cc(-c2cn(C)c(=O)c3cnc(N4CC(F)(F)C4)cc23)cc(OC)c1CN1CCC(CN2CCC3(CC2)CN(c2ccc4c(c2)C(=O)N(C2CCC(=O)N(C)C2=O)C4)C3)CC1] |
| H262 | 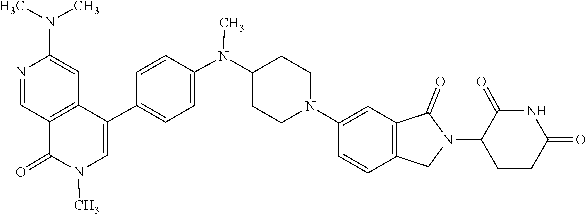 [SMILES: CN(C)c1cc2c(-c3ccc(N(C)C4CCN(c5ccc6c(c5)C(=O)N(C5CCC(=O)NC5=O)C6)CC4)cc3)cn(C)c(=O)c2cn1] |
| H263 | 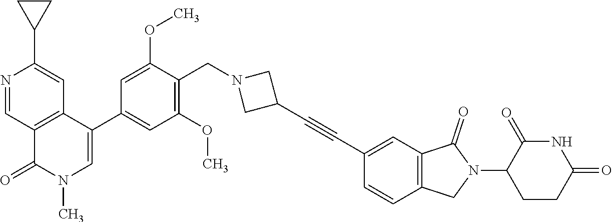 [SMILES: COc1cc(-c2cn(C)c(=O)c3cnc(C4CC4)cc23)cc(OC)c1CN1CC(C#Cc2ccc3c(c2)C(=O)N(C2CCC(=O)NC2=O)C3)C1] |
| H264 | 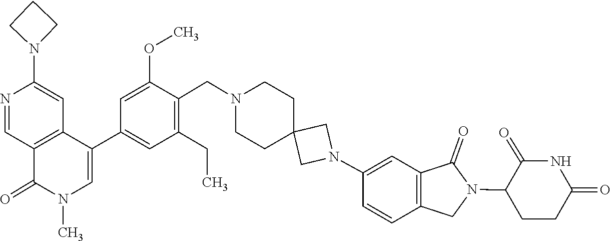 [SMILES: CCc1cc(-c2cn(C)c(=O)c3cnc(N4CCC4)cc23)cc(OC)c1CN1CCC2(CC1)CN(c1ccc3c(c1)C(=O)N(C1CCC(=O)NC1=O)C3)C2] |
| H265 | 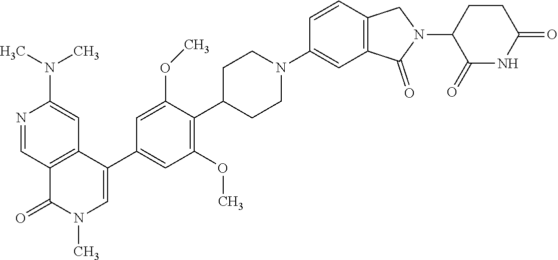 [SMILES: COc1cc(-c2cn(C)c(=O)c3cnc(N(C)C)cc23)cc(OC)c1C1CCN(c2ccc3c(c2)C(=O)N(C2CCC(=O)NC2=O)C3)CC1] |
| H266 | 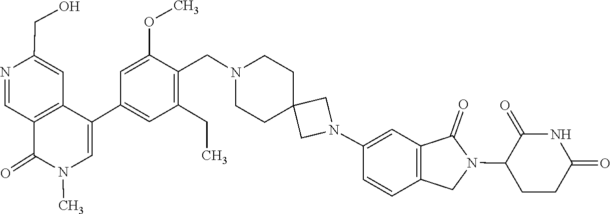 [SMILES: CCc1cc(-c2cn(C)c(=O)c3cnc(CO)cc23)cc(OC)c1CN1CCC2(CC1)CN(c1ccc3c(c1)C(=O)N(C1CCC(=O)NC1=O)C3)C2] |
| H267 | 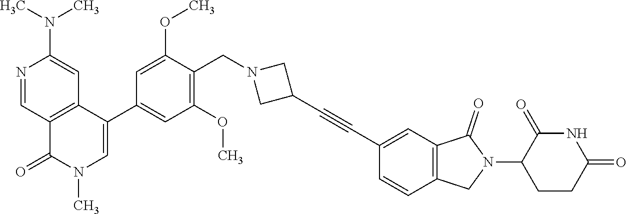 [SMILES: COc1cc(-c2cn(C)c(=O)c3cnc(N(C)C)cc23)cc(OC)c1CN1CC(C#Cc2ccc3c(c2)C(=O)N(C2CCC(=O)NC2=O)C3)C1] |
| H268 | 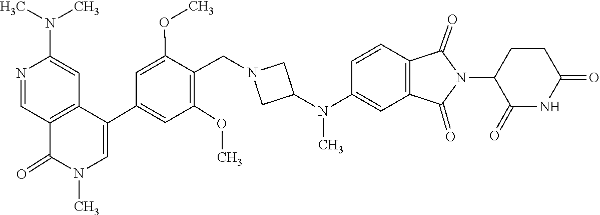 [SMILES: COc1cc(-c2cn(C)c(=O)c3cnc(N(C)C)cc23)cc(OC)c1CN1CC(N(C)c2ccc3c(c2)C(=O)N(C2CCC(=O)NC2=O)C3=O)C1] |
| H269 | 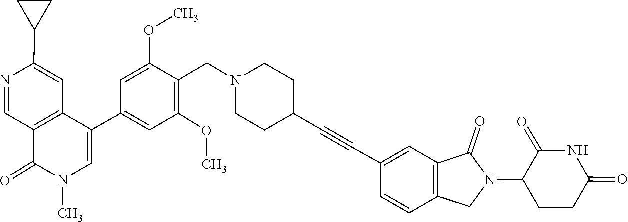 [SMILES: COc1cc(-c2cn(C)c(=O)c3cnc(C4CC4)cc23)cc(OC)c1CN1CCC(C#Cc2ccc3c(c2)C(=O)N(C2CCC(=O)NC2=O)C3)CC1] |
| H270 | 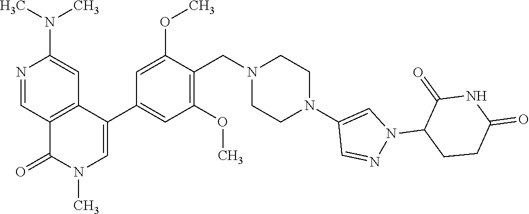 [SMILES: COc1cc(-c2cn(C)c(=O)c3cnc(N(C)C)cc23)cc(OC)c1CN1CCN(c2cnn(C3CCC(=O)NC3=O)c2)CC1] |
| H271 | 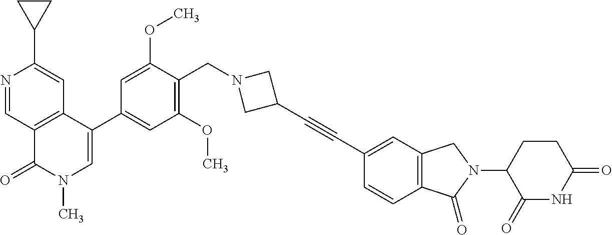 [SMILES: COc1cc(-c2cn(C)c(=O)c3cnc(C4CC4)cc23)cc(OC)c1CN1CC(C#Cc2ccc3c(c2)CN(C2CCC(=O)NC2=O)C3=O)C1] |
| H272 | 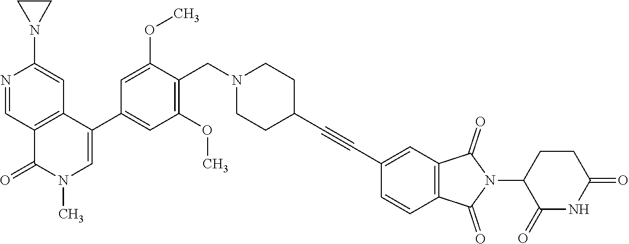 [SMILES: COc1cc(-c2cn(C)c(=O)c3cnc(N4CC4)cc23)cc(OC)c1CN1CCC(C#Cc2ccc3c(c2)C(=O)N(C2CCC(=O)NC2=O)C3=O)CC1] |
| H273 | 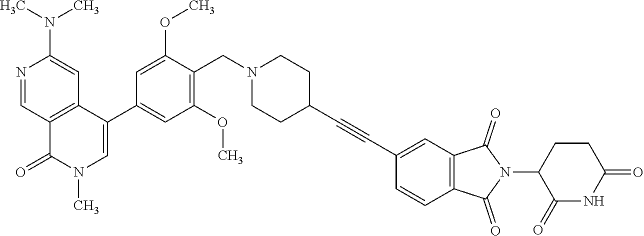 [SMILES: COc1cc(-c2cn(C)c(=O)c3cnc(N(C)C)cc23)cc(OC)c1CN1CCC(C#Cc2ccc3c(c2)C(=O)N(C2CCC(=O)NC2=O)C3=O)CC1] |
| H274 | 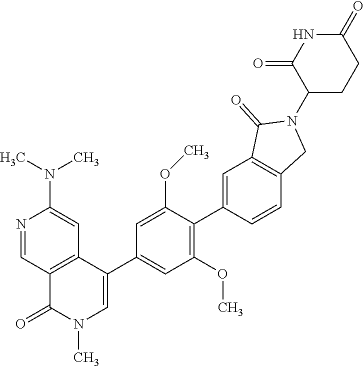 [SMILES: COc1cc(-c2cn(C)c(=O)c3cnc(N(C)C)cc23)cc(OC)c1-c1ccc2c(c1)C(=O)N(C1CCC(=O)NC1=O)C2] |
| H275 | 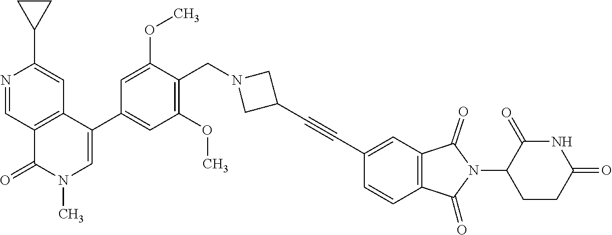 [SMILES: COc1cc(-c2cn(C)c(=O)c3cnc(C4CC4)cc23)cc(OC)c1CN1CC(C#Cc2ccc3c(c2)C(=O)N(C2CCC(=O)NC2=O)C3=O)C1] |
| H276 | 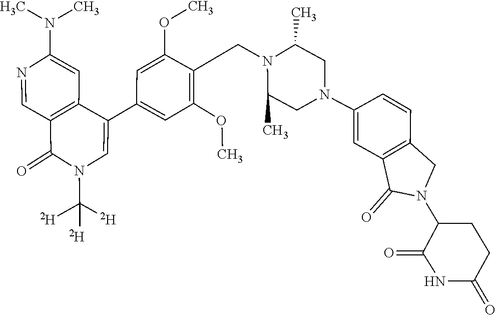 [SMILES: [2H]C([2H])([2H])n1cc(-c2cc(OC)c(CN3[C@H](C)CN(c4ccc5c(c4)C(=O)N(C4CCC(=O)NC4=O)C5)C[C@H]3C)c(OC)c2)c2cc(N(C)C)ncc2c1=O] |
| H277 | 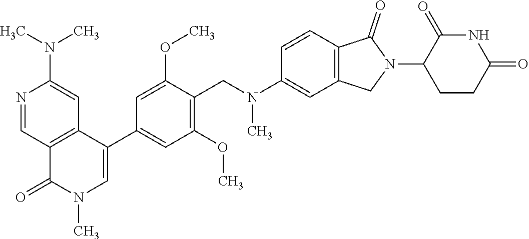 [SMILES: COc1cc(-c2cn(C)c(=O)c3cnc(N(C)C)cc23)cc(OC)c1CN(C)c1ccc2c(c1)CN(C1CCC(=O)NC1=O)C2=O] |
| H278 | 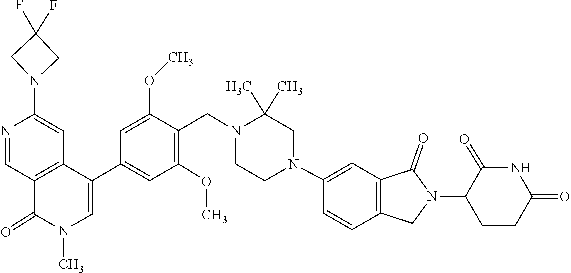 [SMILES: COc1cc(-c2cn(C)c(=O)c3cnc(N4CC(F)(F)C4)cc23)cc(OC)c1CN1CCN(c2ccc3c(c2)C(=O)N(C2CCC(=O)NC2=O)C3)CC1(C)C] |
| H279 | 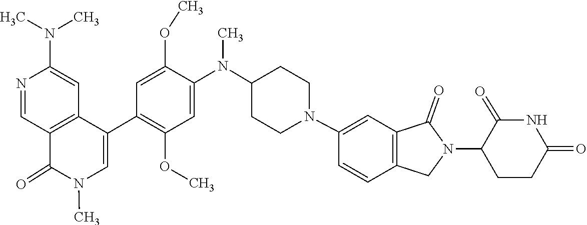 [SMILES: COc1cc(N(C)C2CCN(c3ccc4c(c3)C(=O)N(C3CCC(=O)NC3=O)C4)CC2)c(OC)cc1-c1cn(C)c(=O)c2cnc(N(C)C)cc12] |
| H280 | 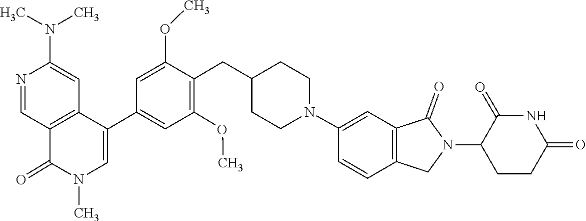 [SMILES: COc1cc(-c2cn(C)c(=O)c3cnc(N(C)C)cc23)cc(OC)c1CC1CCN(c2ccc3c(c2)C(=O)N(C2CCC(=O)NC2=O)C3)CC1] |
| H281 | 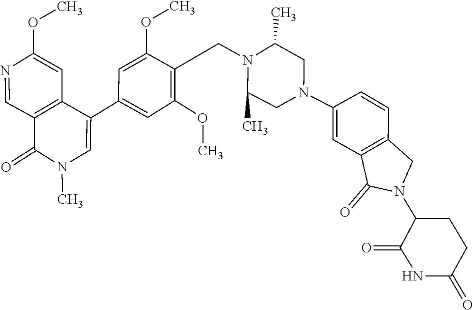 [SMILES: COc1cc2c(-c3cc(OC)c(CN4[C@H](C)CN(c5ccc6c(c5)C(=O)N(C5CCC(=O)NC5=O)C6)C[C@H]4C)c(OC)c3)cn(C)c(=O)c2cn1] |
| H282 | 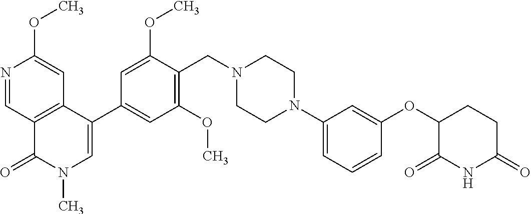 [SMILES: COc1cc2c(-c3cc(OC)c(CN4CCN(c5cccc(OC6CCC(=O)NC6=O)c5)CC4)c(OC)c3)cn(C)c(=O)c2cn1] |
| H283 | 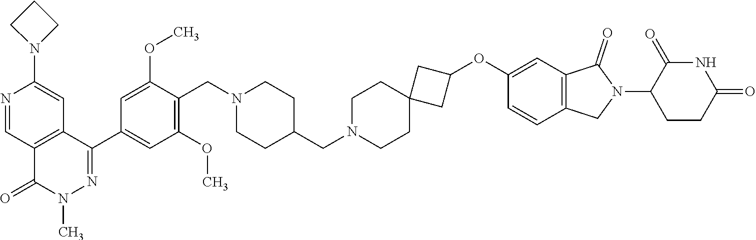 [SMILES: COc1cc(-c2nn(C)c(=O)c3cnc(N4CCC4)cc23)cc(OC)c1CN1CCC(CN2CCC3(CC2)CC(Oc2ccc4c(c2)C(=O)N(C2CCC(=O)NC2=O)C4)C3)CC1] |
| H284 | 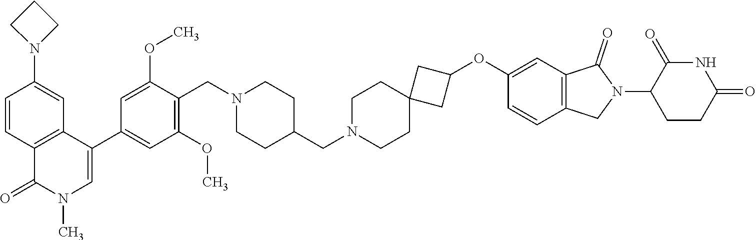 [SMILES: COc1cc(-c2cn(C)c(=O)c3ccc(N4CCC4)cc23)cc(OC)c1CN1CCC(CN2CCC3(CC2)CC(Oc2ccc4c(c2)C(=O)N(C2CCC(=O)NC2=O)C4)C3)CC1] |
| H285 | 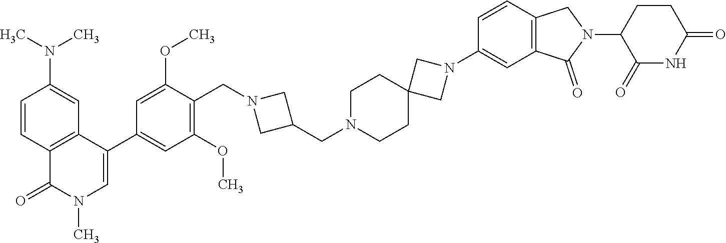 [SMILES: COc1cc(-c2cn(C)c(=O)c3ccc(N(C)C)cc23)cc(OC)c1CN1CC(CN2CCC3(CC2)CN(c2ccc4c(c2)C(=O)N(C2CCC(=O)NC2=O)C4)C3)C1] |
| H286 | 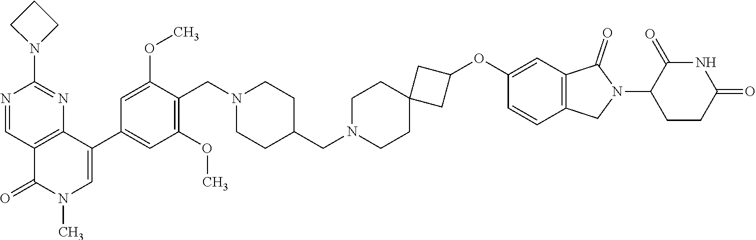 [SMILES: COc1cc(-c2cn(C)c(=O)c3cnc(N4CCC4)nc23)cc(OC)c1CN1CCC(CN2CCC3(CC2)CC(Oc2ccc4c(c2)C(=O)N(C2CCC(=O)NC2=O)C4)C3)CC1] |
| H287 | 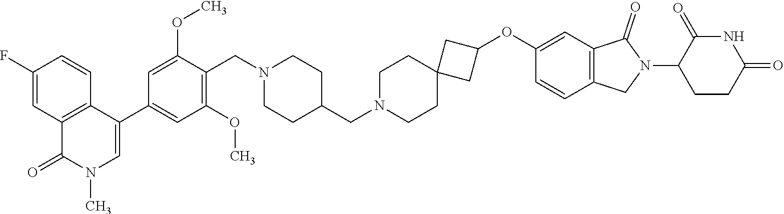 [SMILES: COc1cc(-c2cn(C)c(=O)c3cc(F)ccc23)cc(OC)c1CN1CCC(CN2CCC3(CC2)CC(Oc2ccc4c(c2)C(=O)N(C2CCC(=O)NC2=O)C4)C3)CC1] |
| H288 | 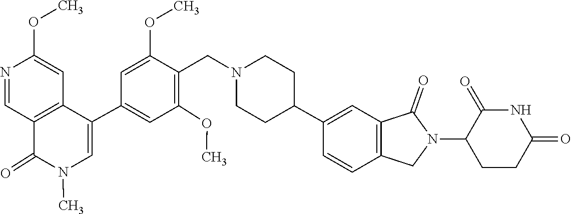 [SMILES: COc1cc2c(-c3cc(OC)c(CN4CCC(c5ccc6c(c5)C(=O)N(C5CCC(=O)NC5=O)C6)CC4)c(OC)c3)cn(C)c(=O)c2cn1] |
| H289 | 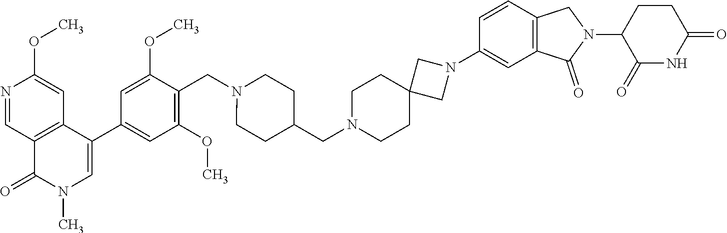 [SMILES: COc1cc2c(-c3cc(OC)c(CN4CCC(CN5CCC6(CC5)CN(c5ccc7c(c5)C(=O)N(C5CCC(=O)NC5=O)C7)C6)CC4)c(OC)c3)cn(C)c(=O)c2cn1] |
| H290 | 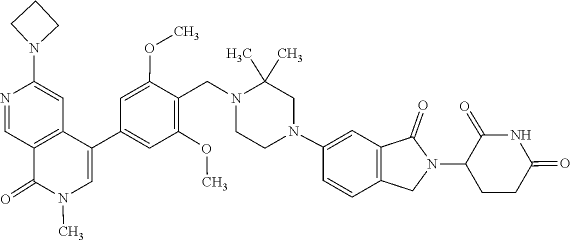 [SMILES: COc1cc(-c2cn(C)c(=O)c3cnc(N4CCC4)cc23)cc(OC)c1CN1CCN(c2ccc3c(c2)C(=O)N(C2CCC(=O)NC2=O)C3)CC1(C)C] |
| H291 | 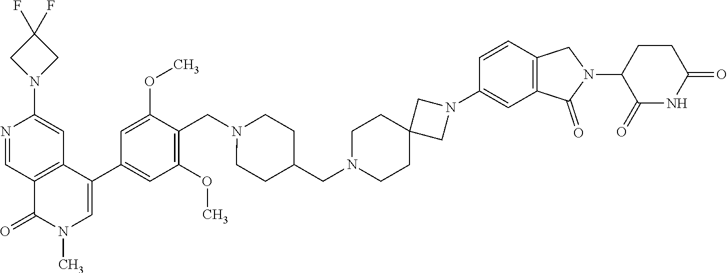 [SMILES: COc1cc(-c2cn(C)c(=O)c3cnc(N4CC(F)(F)C4)cc23)cc(OC)c1CN1CCC(CN2CCC3(CC2)CN(c2ccc4c(c2)C(=O)N(C2CCC(=O)NC2=O)C4)C3)CC1] |
| H292 | 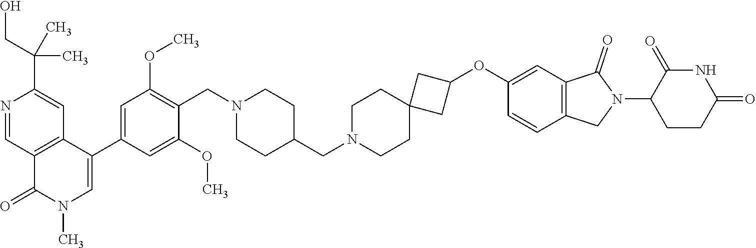 [SMILES: COc1cc(-c2cn(C)c(=O)c3cnc(C(C)(C)CO)cc23)cc(OC)c1CN1CCC(CN2CCC3(CC2)CC(Oc2ccc4c(c2)C(=O)N(C2CCC(=O)NC2=O)C4)C3)CC1] |
| H293 | 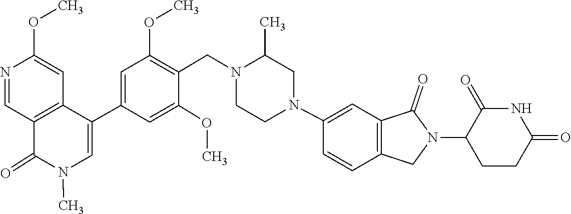 [SMILES: COc1cc2c(-c3cc(OC)c(CN4CCN(c5ccc6c(c5)C(=O)N(C5CCC(=O)NC5=O)C6)CC4C)c(OC)c3)cn(C)c(=O)c2cn1] |
| H294 | 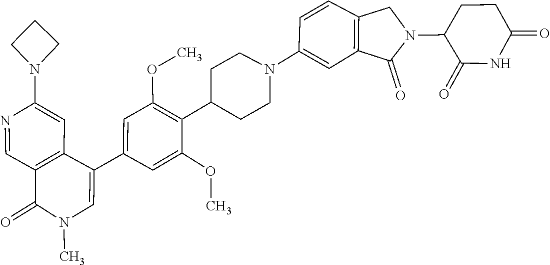 [SMILES: COc1cc(-c2cn(C)c(=O)c3cnc(N4CCC4)cc23)cc(OC)c1C1CCN(c2ccc3c(c2)C(=O)N(C2CCC(=O)NC2=O)C3)CC1] |
| H295 | 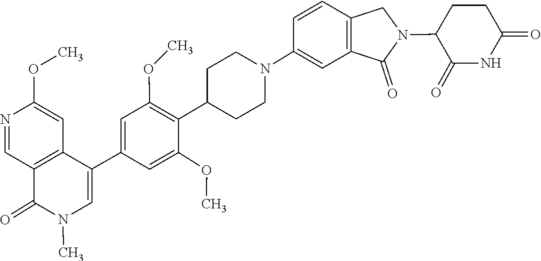 [SMILES: COc1cc2c(-c3cc(OC)c(C4CCN(c5ccc6c(c5)C(=O)N(C5CCC(=O)NC5=O)C6)CC4)c(OC)c3)cn(C)c(=O)c2cn1] |
| H296 | 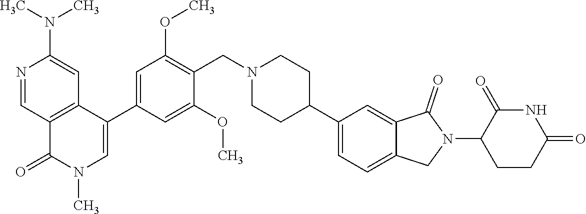 [SMILES: COc1cc(-c2cn(C)c(=O)c3cnc(N(C)C)cc23)cc(OC)c1CN1CCC(c2ccc3c(c2)C(=O)N(C2CCC(=O)NC2=O)C3)CC1] |
| H297 | 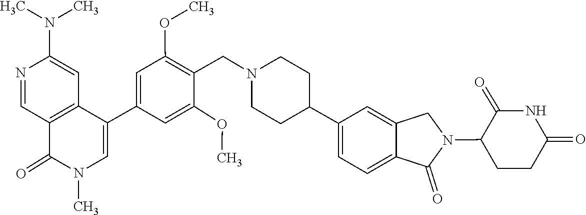 [SMILES: COc1cc(-c2cn(C)c(=O)c3cnc(N(C)C)cc23)cc(OC)c1CN1CCC(c2ccc3c(c2)CN(C2CCC(=O)NC2=O)C3=O)CC1] |
| H298 | 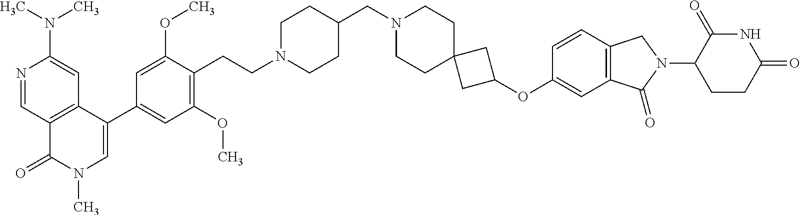 [SMILES: COc1cc(-c2cn(C)c(=O)c3cnc(N(C)C)cc23)cc(OC)c1CCN1CCC(CN2CCC3(CC2)CC(Oc2ccc4c(c2)C(=O)N(C2CCC(=O)NC2=O)C4)C3)CC1] |
| H299 | 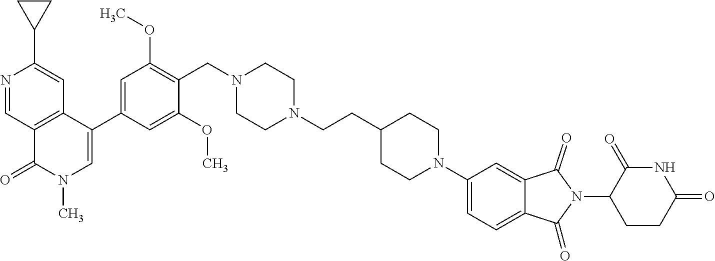 [SMILES: COc1cc(-c2cn(C)c(=O)c3cnc(C4CC4)cc23)cc(OC)c1CN1CCN(CCC2CCN(c3ccc4c(c3)C(=O)N(C3CCC(=O)NC3=O)C4=O)CC2)CC1] |
| H300 | 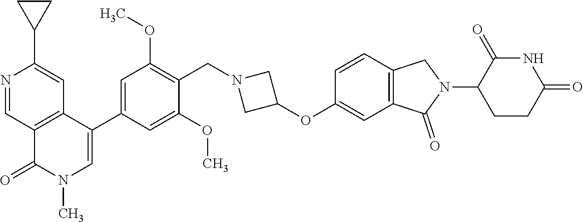 [SMILES: COc1cc(-c2cn(C)c(=O)c3cnc(C4CC4)cc23)cc(OC)c1CN1CC(Oc2ccc3c(c2)C(=O)N(C2CCC(=O)NC2=O)C3)C1] |
| H301 | 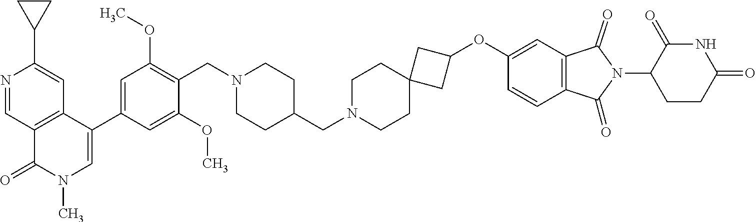 [SMILES: COc1cc(-c2cn(C)c(=O)c3cnc(C4CC4)cc23)cc(OC)c1CN1CCC(CN2CCC3(CC2)CC(Oc2ccc4c(c2)C(=O)N(C2CCC(=O)NC2=O)C4=O)C3)CC1] |
| H302 | 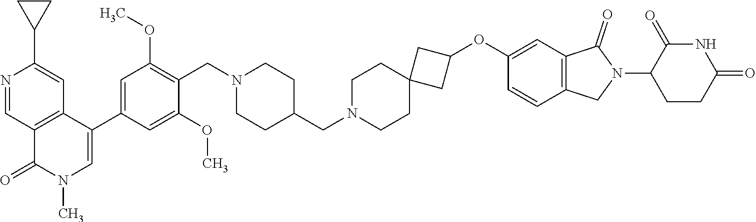 [SMILES: COc1cc(-c2cn(C)c(=O)c3cnc(C4CC4)cc23)cc(OC)c1CN1CCC(CN2CCC3(CC2)CC(Oc2ccc4c(c2)C(=O)N(C2CCC(=O)NC2=O)C4)C3)CC1] |
| H303 | 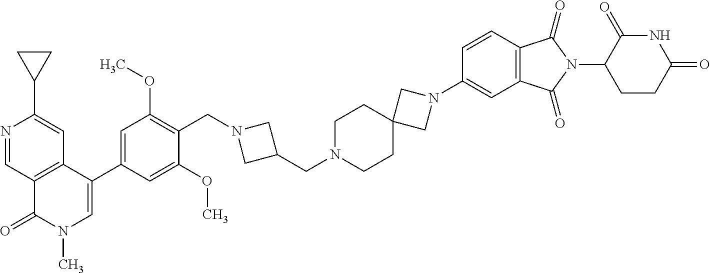 [SMILES: COc1cc(-c2cn(C)c(=O)c3cnc(C4CC4)cc23)cc(OC)c1CN1CC(CN2CCC3(CC2)CN(c2ccc4c(c2)C(=O)N(C2CCC(=O)NC2=O)C4=O)C3)C1] |
| H304 | 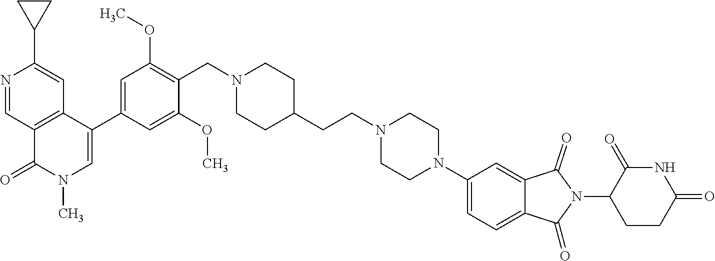 [SMILES: COc1cc(-c2cn(C)c(=O)c3cnc(C4CC4)cc23)cc(OC)c1CN1CCC(CCN2CCN(c3ccc4c(c3)C(=O)N(C3CCC(=O)NC3=O)C4=O)CC2)CC1] |
| H305 | 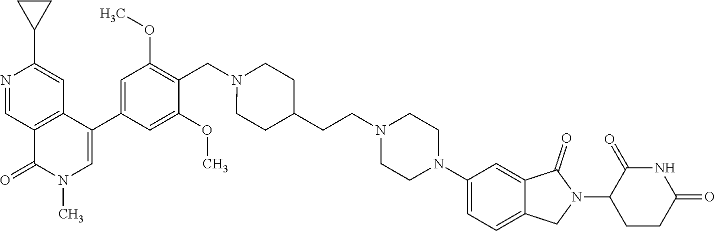 [SMILES: COc1cc(-c2cn(C)c(=O)c3cnc(C4CC4)cc23)cc(OC)c1CN1CCC(CCN2CCN(c3ccc4c(c3)C(=O)N(C3CCC(=O)NC3=O)C4)CC2)CC1] |
| H306 | 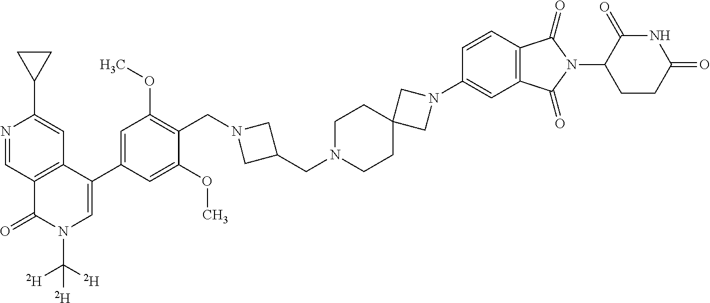 [SMILES: [2H]C([2H])([2H])n1cc(-c2cc(OC)c(CN3CC(CN4CCC5(CC4)CN(c4ccc6c(c4)C(=O)N(C4CCC(=O)NC4=O)C6=O)C5)C3)c(OC)c2)c2cc(C3CC3)ncc2c1=O] |
| H307 | 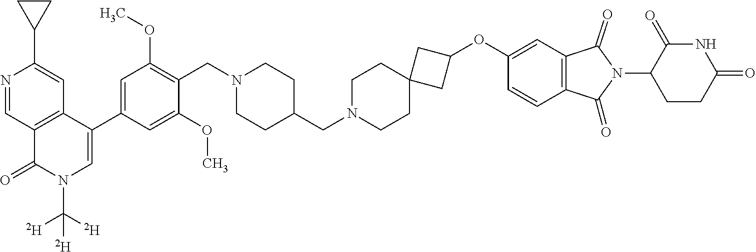 [SMILES: [2H]C([2H])([2H])n1cc(-c2cc(OC)c(CN3CCC(CN4CCC5(CC4)CC(Oc4ccc6c(c4)C(=O)N(C4CCC(=O)NC4=O)C6=O)C5)CC3)c(OC)c2)c2cc(C3CC3)ncc2c1=O] |
| H308 | 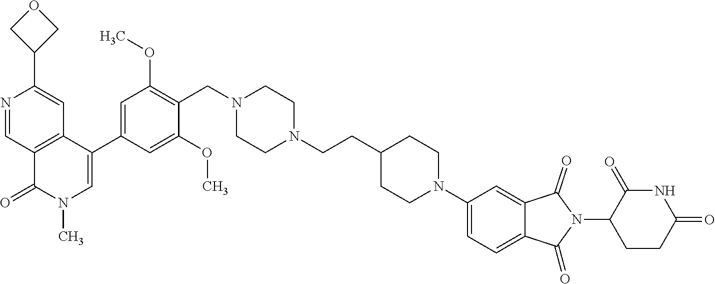 [SMILES: COc1cc(-c2cn(C)c(=O)c3cnc(C4COC4)cc23)cc(OC)c1CN1CCN(CCC2CCN(c3ccc4c(c3)C(=O)N(C3CCC(=O)NC3=O)C4=O)CC2)CC1] |
| H309 | 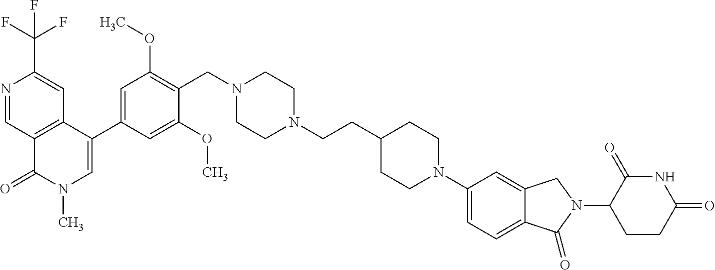 [SMILES: COc1cc(-c2cn(C)c(=O)c3cnc(C(F)(F)F)cc23)cc(OC)c1CN1CCN(CCC2CCN(c3ccc4c(c3)CN(C3CCC(=O)NC3=O)C4=O)CC2)CC1] |
| H310 | 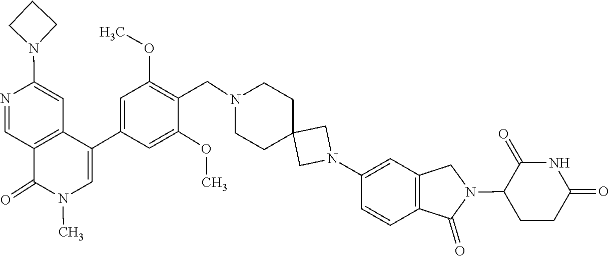 [SMILES: COc1cc(-c2cn(C)c(=O)c3cnc(N4CCC4)cc23)cc(OC)c1CN1CCC2(CC1)CN(c1ccc3c(c1)CN(C1CCC(=O)NC1=O)C3=O)C2] |
| H311 | 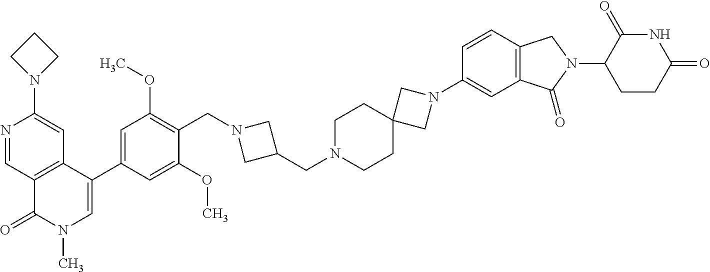 [SMILES: COc1cc(-c2cn(C)c(=O)c3cnc(N4CCC4)cc23)cc(OC)c1CN1CC(CN2CCC3(CC2)CN(c2ccc4c(c2)C(=O)N(C2CCC(=O)NC2=O)C4)C3)C1] |
| H312 | 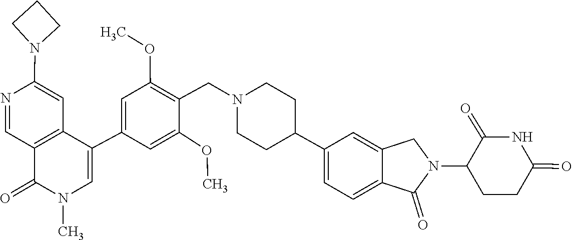 [SMILES: COc1cc(-c2cn(C)c(=O)c3cnc(N4CCC4)cc23)cc(OC)c1CN1CCC(c2ccc3c(c2)CN(C2CCC(=O)NC2=O)C3=O)CC1] |
| H313 | 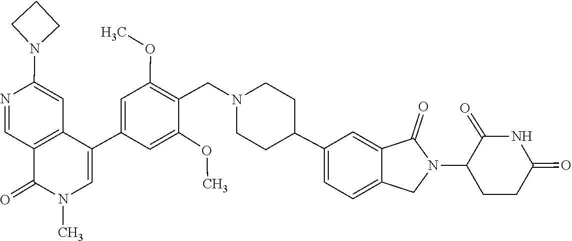 [SMILES: COc1cc(-c2cn(C)c(=O)c3cnc(N4CCC4)cc23)cc(OC)c1CN1CCC(c2ccc3c(c2)C(=O)N(C2CCC(=O)NC2=O)C3)CC1] |
| H314 | 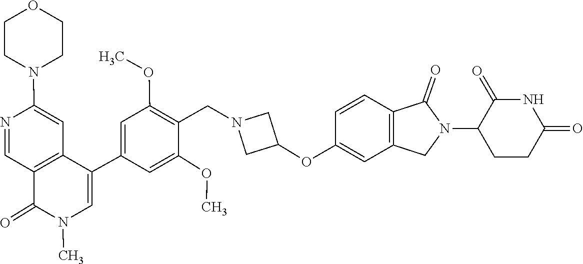 [SMILES: COc1cc(-c2cn(C)c(=O)c3cnc(N4CCOCC4)cc23)cc(OC)c1CN1CC(Oc2ccc3c(c2)CN(C2CCC(=O)NC2=O)C3=O)C1] |
| H315 | 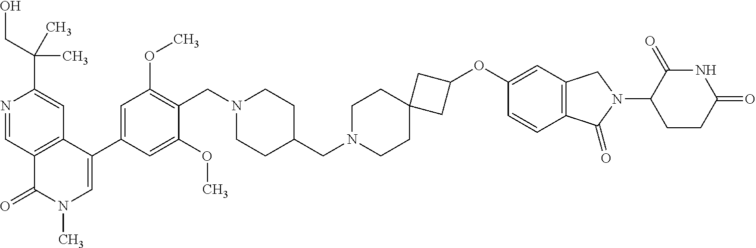 [SMILES: COc1cc(-c2cn(C)c(=O)c3cnc(C(C)(C)CO)cc23)cc(OC)c1CN1CCC(CN2CCC3(CC2)CC(Oc2ccc4c(c2)CN(C2CCC(=O)NC2=O)C4=O)C3)CC1] |
| H316 | 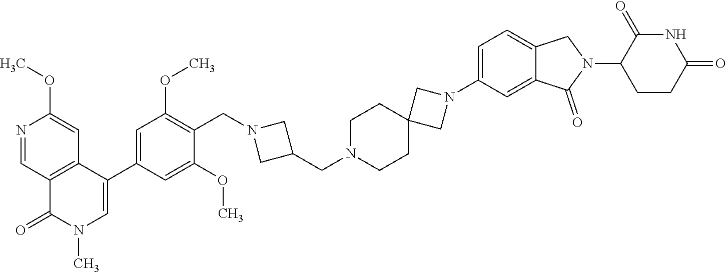 [SMILES: COc1cc2c(-c3cc(OC)c(CN4CC(CN5CCC6(CC5)CN(c5ccc7c(c5)C(=O)N(C5CCC(=O)NC5=O)C7)C6)C4)c(OC)c3)cn(C)c(=O)c2cn1] |

or a pharmaceutically acceptable salt thereof.

The BRD9 inhibitor may be, e.g., a compound selected from the group consisting of:

| Cmpd | |
|---|---|
| # | Structure & Name |
| 200 | 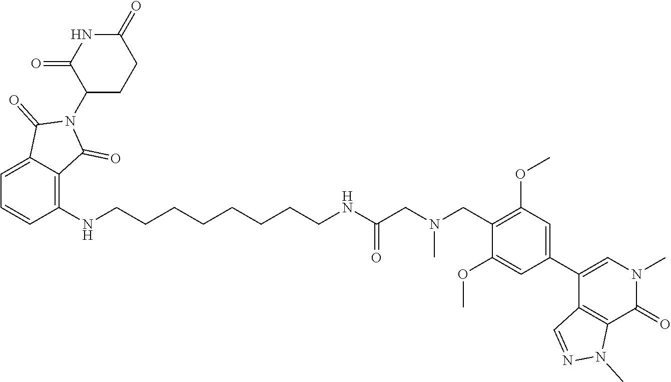 [SMILES: COc1cc(-c2cn(C)c(=O)c3c2cnn3C)cc(OC)c1CN(C)CC(=O)NCCCCCCCCNc1cccc2c1C(=O)N(C1CCC(=O)NC1=O)C2=O] |
| | 2-((4-(1,6-dimethyl-7-oxo-6,7-dihydro-1H-pyrazolo[3,4-c]pyridin-4-yl)-2,6- |
| | dimethoxybenzyl)(methyl)amino)-N-(8-((2-(2,6-dioxopiperidin-3-yl)-1,3- |
| | dioxoisoindolin-4-yl)amino)octyl)acetamide |
| 201 | 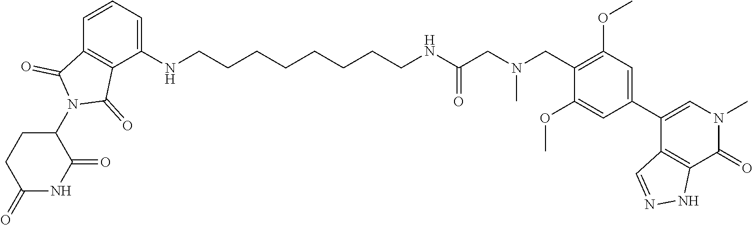 [SMILES: COc1cc(-c2cn(C)c(=O)c3[nH]ncc23)cc(OC)c1CN(C)CC(=O)NCCCCCCCCNc1cccc2c1C(=O)N(C1CCC(=O)NC1=O)C2=O] |
| | 2-((2,6-dimethoxy-4-(6-methyl-7-oxo-6,7-dihydro-1H-pyrazolo[3,4-c]pyridin- |
| | 4-yl)benzyl)(methyl)amino)-N-(8-((2-(2,6-dioxopiperidin-3-yl)-1,3- |
| | dioxoisoindolin-4-yl)amino)octyl)acetamide |
| 202 | 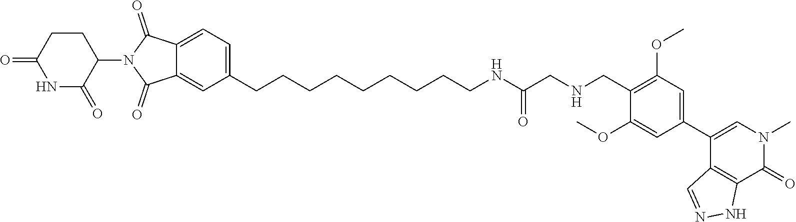 [SMILES: COc1cc(-c2cn(C)c(=O)c3[nH]ncc23)cc(OC)c1CNCC(=O)NCCCCCCCCCc1ccc2c(c1)C(=O)N(C1CCC(=O)NC1=O)C2=O] |
| | 2-((2,6-dimethoxy-4-(6-methyl-7-oxo-6,7-dihydro-1H-pyrazolo[3,4-c]pyridin- |
| | 4-yl)benzyl)amino)-N-(9-(2-(2,6-dioxopiperidin-3-yl)-1,3-dioxoisoindolin-5- |
| | yl)nonyl)acetamide |
| 203 | 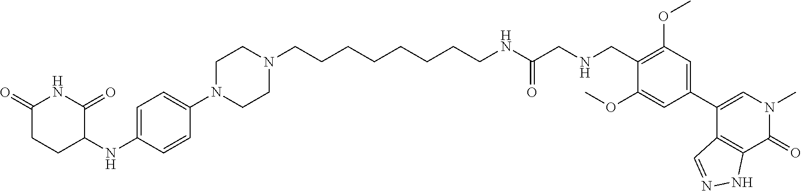 [SMILES: COc1cc(-c2cn(C)c(=O)c3[nH]ncc23)cc(OC)c1CNCC(=O)NCCCCCCCCN1CCN(c2ccc(NC3CCC(=O)NC3=O)cc2)CC1] |
| | 2-((2,6-dimethoxy-4-(6-methyl-7-oxo-6,7-dihydro-1H-pyrazolo[3,4-c]pyridin- |
| | 4-yl)benzyl)amino)-N-(8-(4-(4-((2,6-dioxopiperidin-3- |
| | yl)amino)phenyl)piperazin-1-yl)octyl)acetamide |
| 204 | 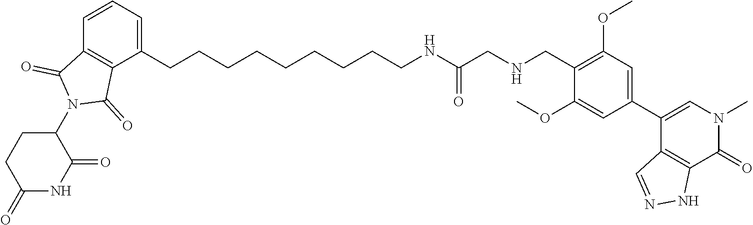 [SMILES: COc1cc(-c2cn(C)c(=O)c3[nH]ncc23)cc(OC)c1CNCC(=O)NCCCCCCCCCc1cccc2c1C(=O)N(C1CCC(=O)NC1=O)C2=O] |
| | 2-((2,6-dimethoxy-4-(6-methyl-7-oxo-6,7-dihydro-1H-pyrazolo[3,4-c]pyridin- |
| | 4-yl)benzyl)amino)-N-(9-(2-(2,6-dioxopiperidin-3-yl)-1,3-dioxoisoindolin-4- |
| | yl)nonyl)acetamide |
| 206 | 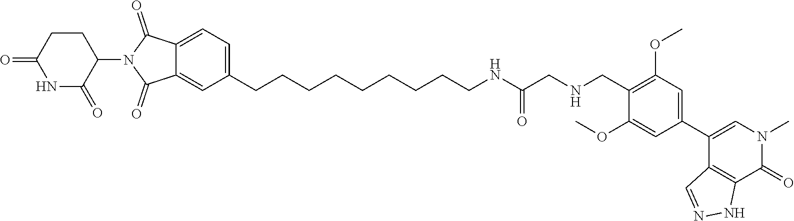 [SMILES: COc1cc(-c2cn(C)c(=O)c3[nH]ncc23)cc(OC)c1CNCC(=O)NCCCCCCCCCc1ccc2c(c1)C(=O)N(C1CCC(=O)NC1=O)C2=O] |
| | 2-((4-(1,6-dimethyl-7-oxo-6,7-dihydro-1H-pyrazolo[3,4-c]pyridin-4-yl)-2,6- |
| | dimethoxybenzyl)amino)-N-(9-(2-(2,6-dioxopiperidin-3-yl)-1,3- |
| | dioxoisoindolin-5-yl)nonyl)acetamide |
| 207 | 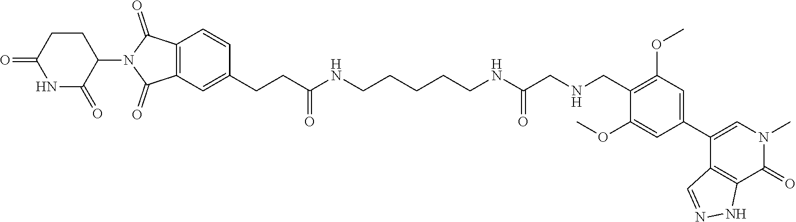 [SMILES: COc1cc(-c2cn(C)c(=O)c3[nH]ncc23)cc(OC)c1CNCC(=O)NCCCCCNC(=O)CCc1ccc2c(c1)C(=O)N(C1CCC(=O)NC1=O)C2=O] |
| | N-(5-(2-((2,6-dimethoxy-4-(6-methyl-7-oxo-6,7-dihydro-1H-pyrazolo[3,4- |
| | c]pyridin-4-yl)benzyl)amino)acetamido)pentyl)-3-(2-(2,6-dioxopiperidin-3-yl)- |
| | 1,3-dioxoisoindolin-5-yl)propanamide |
| 208 | 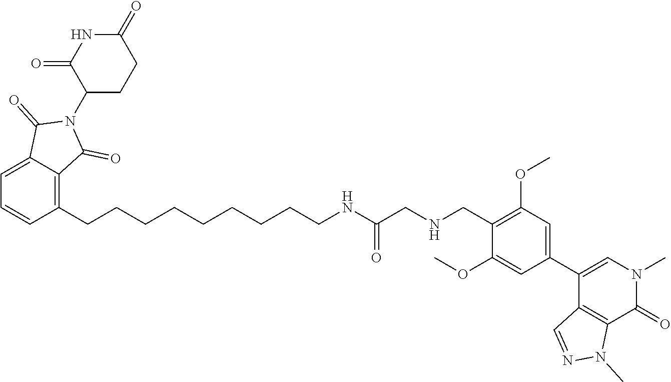 [SMILES: COc1cc(-c2cn(C)c(=O)c3c2cnn3C)cc(OC)c1CNCC(=O)NCCCCCCCCCc1cccc2c1C(=O)N(C1CCC(=O)NC1=O)C2=O] |
| | 2-((4-(1,6-dimethyl-7-oxo-6,7-dihydro-1H-pyrazolo[3,4-c]pyridin-4-yl)-2,6- |
| | dimethoxybenzyl)amino)-N-(9-(2-(2,6-dioxopiperidin-3-yl)-1,3- |
| | dioxoisoindolin-4-yl)nonyl)acetamide |
| 209 | 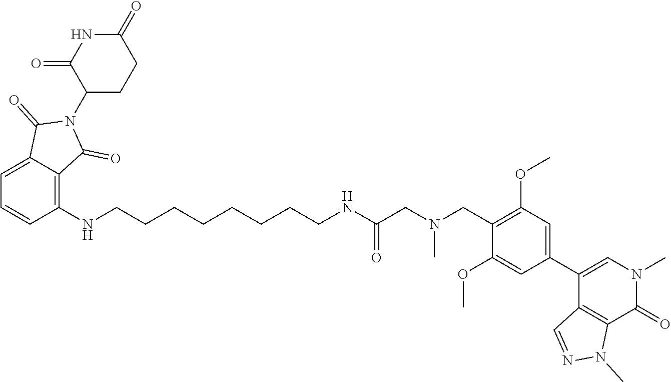 [SMILES: COc1cc(-c2cn(C)c(=O)c3c2cnn3C)cc(OC)c1CN(C)CC(=O)NCCCCCCCCNc1cccc2c1C(=O)N(C1CCC(=O)NC1=O)C2=O] |
| | 2-((4-(1,6-dimethyl-7-oxo-6,7-dihydro-1H-pyrrolo[2,3-c]pyridin-4-yl)-2,6- |
| | dimethoxybenzyl)(methyl)amino)-N-(8-((2-(2,6-dioxopiperidin-3-yl)-1,3- |
| | dioxoisoindolin-4-yl)amino)octyl)acetamide |
| 218 | 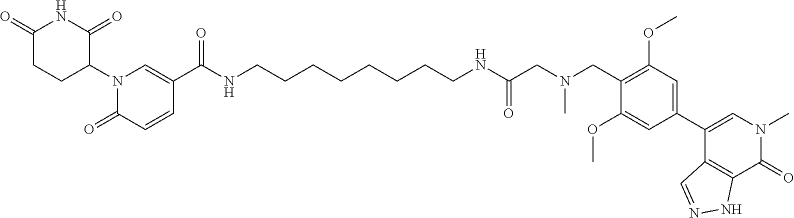 [SMILES: COc1cc(-c2cn(C)c(=O)c3[nH]ncc23)cc(OC)c1CN(C)CC(=O)NCCCCCCCCNC(=O)c1ccc(=O)n(C2CCC(=O)NC2=O)c1] |
| | N-(8-(2-((4-(1,6-dimethyl-7-oxo-6,7-dihydro-1H-pyrrolo[2,3-c]pyridin-4-yl)- |
| | 2,6-dimethoxybenzyl)(methyl)amino)acetamido)octyl)-1-(2,6-dioxopiperidin-3- |
| | yl)-6-oxo-1,6-dihydropyridine-3-carboxamide |
| 220 | 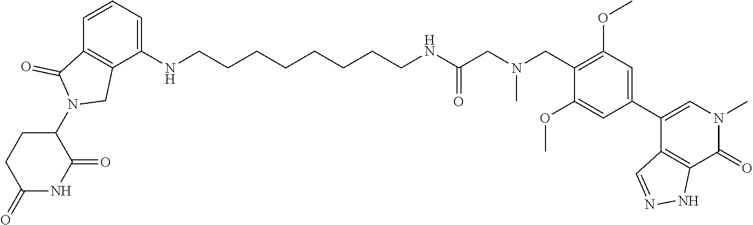 [SMILES: COc1cc(-c2cn(C)c(=O)c3[nH]ncc23)cc(OC)c1CN(C)CC(=O)NCCCCCCCCNc1cccc2c1CN(C1CCC(=O)NC1=O)C2=O] |
| | 2-((4-(1,6-dimethyl-7-oxo-6,7-dihydro-1H-pyrrolo[2,3-c]pyridin-4-yl)-2,6- |
| | dimethoxybenzyl)(methyl)amino)-N-(8-((2-(2,6-dioxopiperidin-3-yl)-1- |
| | oxoisoindolin-4-yl)amino)octyl)acetamide |
| 221 | 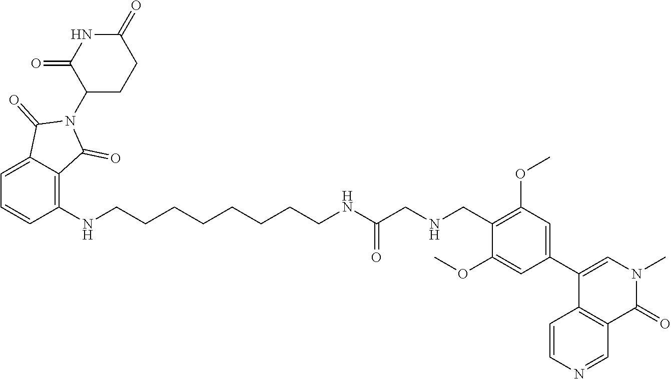 [SMILES: COc1cc(-c2cn(C)c(=O)c3cnccc23)cc(OC)c1CNCC(=O)NCCCCCCCCNc1cccc2c1C(=O)N(C1CCC(=O)NC1=O)C2=O] |
| | 2-((2,6-dimethoxy-4-(2-methyl-1-oxo-1,2-dihydro-2,7-naphthyridin-4- |
| | yl)benzyl)amino)-N-(8-((2-(2,6-dioxopiperidin-3-yl)-1,3-dioxoisoindolin-4- |
| | yl)amino)octyl)acetamide |
| 222 | 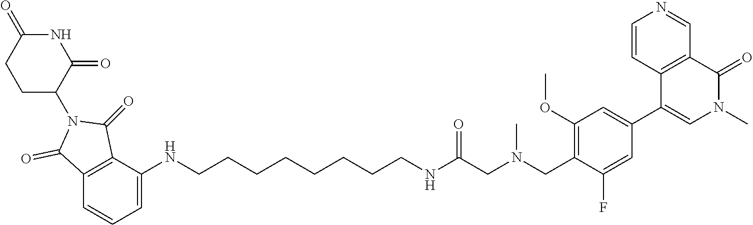 [SMILES: COc1cc(-c2cn(C)c(=O)c3cnccc23)cc(F)c1CN(C)CC(=O)NCCCCCCCCNc1cccc2c1C(=O)N(C1CCC(=O)NC1=O)C2=O] |
| | N-(8-((2-(2,6-dioxopiperidin-3-yl)-1,3-dioxoisoindolin-4-yl)amino)octyl)-2-((2- |
| | fluoro-6-methoxy-4-(2-methyl-1-oxo-1,2-dihydro-2,7-naphthyridin-4- |
| | yl)benzyl)(methyl)amino)acetamide |
| 223 | 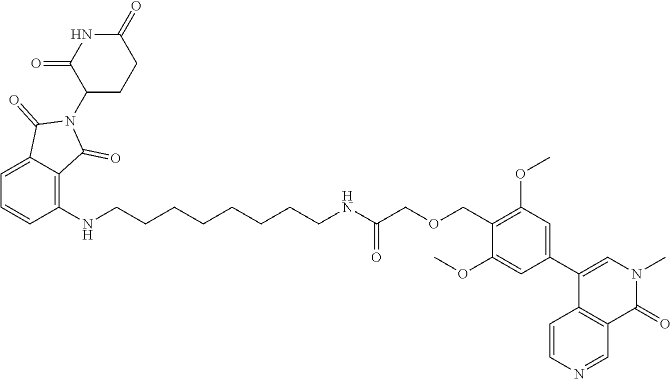 [SMILES: COc1cc(-c2cn(C)c(=O)c3cnccc23)cc(OC)c1COCC(=O)NCCCCCCCCNc1cccc2c1C(=O)N(C1CCC(=O)NC1=O)C2=O] |
| | 2-((2,6-dimethoxy-4-(2-methyl-1-oxo-1,2-dihydro-2,7-naphthyridin-4- |
| | yl)benzyl)oxy)-N-(8-((2-(2,6-dioxopiperidin-3-yl)-1,3-dioxoisoindolin-4- |
| | yl)amino)octyl)acetamide |
| 224 | 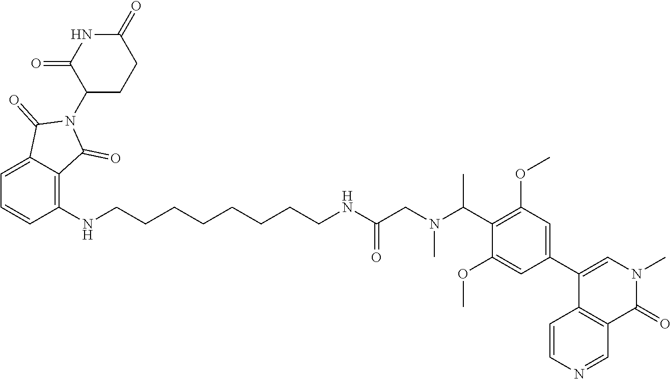 [SMILES: COc1cc(-c2cn(C)c(=O)c3cnccc23)cc(OC)c1C(C)N(C)CC(=O)NCCCCCCCCNc1cccc2c1C(=O)N(C1CCC(=O)NC1=O)C2=O] |
| | 2-((1-(2,6-dimethoxy-4-(2-methyl-1-oxo-1,2-dihydro-2,7-naphthyridin-4- |
| | yl)phenyl)ethyl)(methyl)amino)-N-(8-((2-(2,6-dioxopiperidin-3-yl)-1,3- |
| | dioxoisoindolin-4-yl)amino)octyl)acetamide |
| 225 | 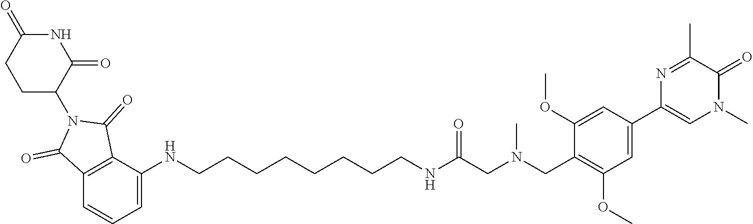 [SMILES: COc1cc(-c2cn(C)c(=O)c(C)n2)cc(OC)c1CN(C)CC(=O)NCCCCCCCCNc1cccc2c1C(=O)N(C1CCC(=O)NC1=O)C2=O] |
| | 2-((4-(4,6-dimethyl-5-oxo-4,5-dihydropyrazin-2-yl)-2,6- |
| | dimethoxybenzyl)(methyl)amino)-N-(8-((2-(2,6-dioxopiperidin-3-yl)-1,3- |
| | dioxoisoindolin-4-yl)amino)octyl)acetamide |
| 300 | 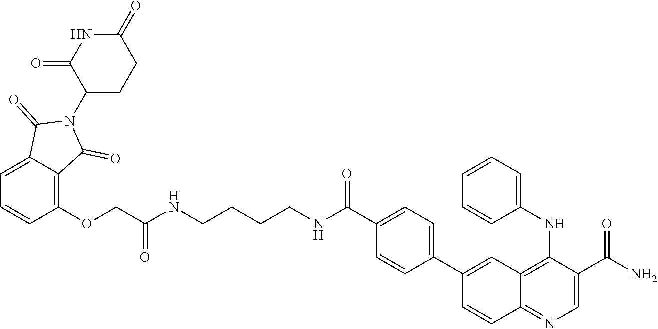 [SMILES: NC(=O)c1cnc2ccc(-c3ccc(C(=O)NCCCCNC(=O)COc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)oxy)acetamido)butyl)carbamoyl)phenyl)-4-(phenylamino)quinoline-3- |
| | carboxamide |
| 301 | 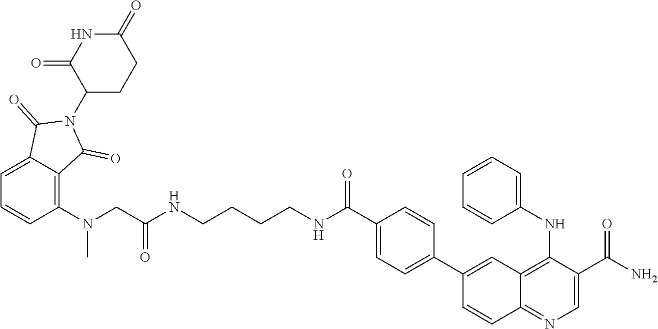 [SMILES: CN(CC(=O)NCCCCNC(=O)c1ccc(-c2ccc3ncc(C(N)=O)c(Nc4ccccc4)c3c2)cc1)c1cccc2c1C(=O)N(C1CCC(=O)NC1=O)C2=O] |
| | 6-(4-((4-(2-((2-(2,6-dioxopiperidin-3-yl)-1,3-dioxoisoindolin-4- |
| | yl)(methyl)amino)acetamido)butyl)carbamoyl)phenyl)-4- |
| | (phenylamino)quinoline-3-carboxamide |
| 302 | 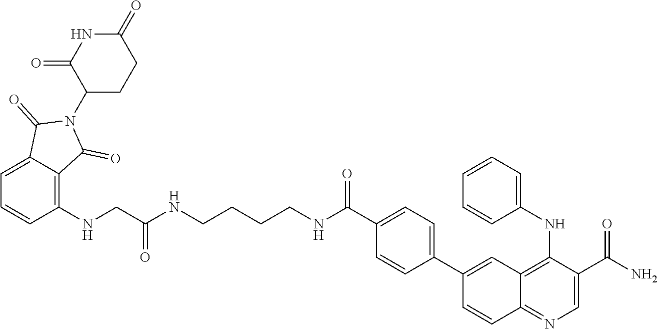 [SMILES: NC(=O)c1cnc2ccc(-c3ccc(C(=O)NCCCCNC(=O)CNc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)amino)acetamido)butyl)carbamoyl)phenyl)-4- |
| | (phenylamino)quinoline-3-carboxamide |
| 303 | 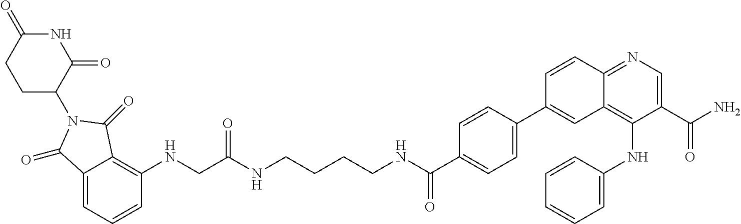 [SMILES: NC(=O)c1cnc2ccc(-c3ccc(C(=O)NCCCCNC(=O)CNc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1-oxoisoindolin-4- |
| | yl)amino)acetamido)butyl)carbamoyl)phenyl)-4- |
| | (phenylamino)quinoline-3-carboxamide |
| 306 | 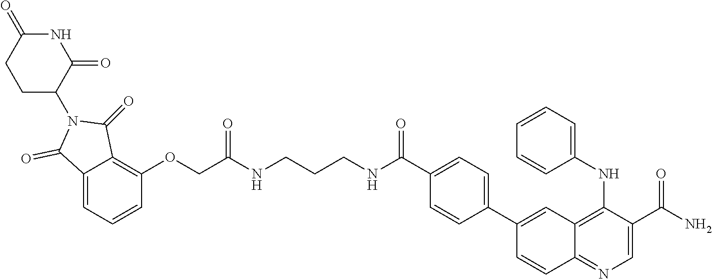 [SMILES: NC(=O)c1cnc2ccc(-c3ccc(C(=O)NCCCNC(=O)COc4cccc5c4C(=O)N(C4CCC(=O)NC4=O)C5=O)cc3)cc2c1Nc1ccccc1] |
| | 6-(4-((3-(2-((2-(2,6-dioxopiperidin-3-yl)-1,3-dioxoisoindolin-4- |
| | yl)oxy)acetamido)propyl)carbamoyl)phenyl)-4-(phenylamino)quinoline- |
| | 3-carboxamide |
| 307 | 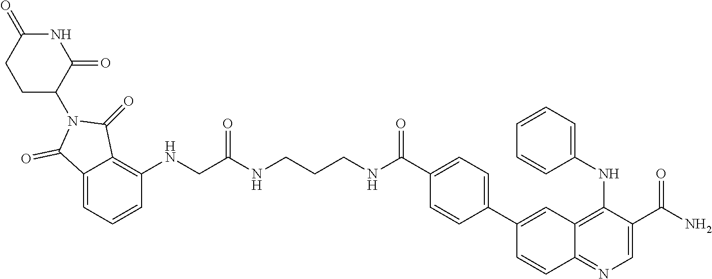 [SMILES: NC(=O)c1cnc2ccc(-c3ccc(C(=O)NCCCNC(=O)CNc4cccc5c4C(=O)N(C4CCC(=O)NC4=O)C5=O)cc3)cc2c1Nc1ccccc1] |
| | 6-(4-((3-(2-((2-(2,6-dioxopiperidin-3-yl)-1,3-dioxoisoindolin-4- |
| | yl)amino)acetamido)propyl)carbamoyl)phenyl)-4- |
| | (phenylamino)quinoline-3-carboxamide |
| 308 | 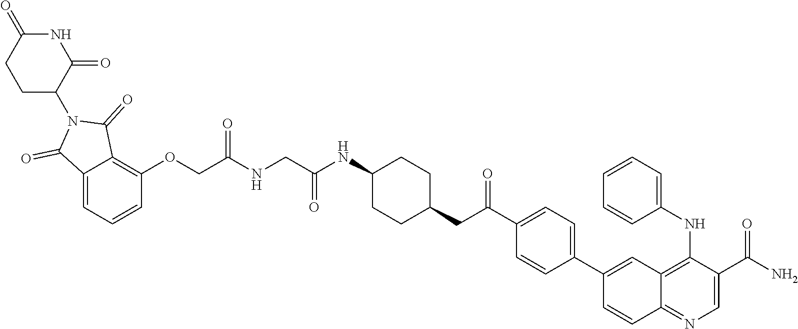 [SMILES: NC(=O)c1cnc2ccc(-c3ccc(C(=O)C[C@H]4CC[C@@H](NC(=O)CNC(=O)COc5cccc6c5C(=O)N(C5CCC(=O)NC5=O)C6=O)CC4)cc3)cc2c1Nc1ccccc1] |
| | 6-(4-(((1s,4s)-4-(2-(2-((2-(2,6-dioxopiperidin-3-yl)-1,3-dioxoisoindolin- |
| | 4-yl)oxy)acetamido)acetamido)cyclohexyl)carbamoyl)phenyl)-4- |
| | (phenylamino)quinoline-3-carboxamide |
| 309 | 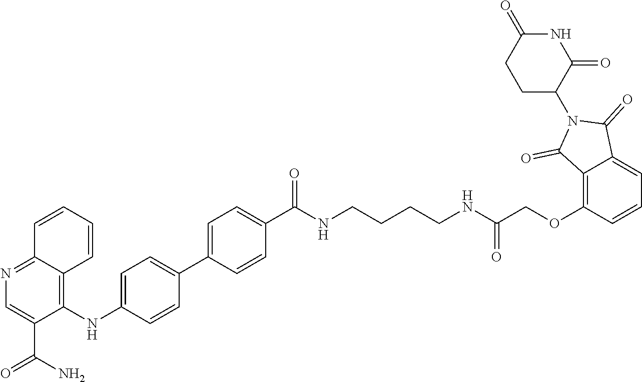 [SMILES: NC(=O)c1cnc2ccccc2c1Nc1ccc(-c2ccc(C(=O)NCCCCNC(=O)COc3cccc4c3C(=O)N(C3CCC(=O)NC3=O)C4=O)cc2)cc1] |
| | 4-((4′-((4-(2-((2-(2,6-dioxopiperidin-3-yl)-1,3-dioxoisoindolin-4- |
| | yl)oxy)acetamido)butyl)carbamoyl)-[1,1′-biphenyl]-4- |
| | yl)amino)quinoline-3-carboxamide |
| 310 | 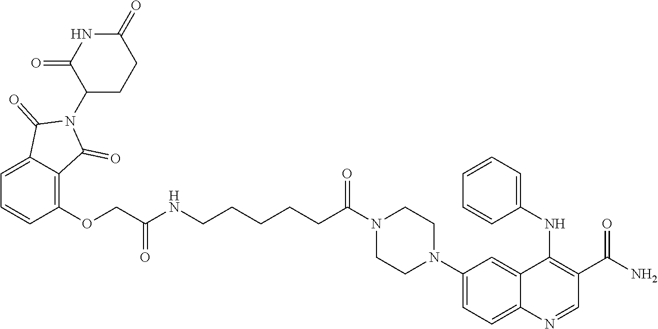 [SMILES: NC(=O)c1cnc2ccc(N3CCN(C(=O)CCCCCNC(=O)COc4cccc5c4C(=O)N(C4CCC(=O)NC4=O)C5=O)CC3)cc2c1Nc1ccccc1] |
| | 6-(4-(6-(2-((2-(2,6-dioxopiperidin-3-yl)-1,3-dioxoisoindolin-4- |
| | yl)oxy)acetamido)hexanoyl)piperazin-1-yl)-4-(phenylamino)quinoline-3- |
| | carboxamide |
| 311 | 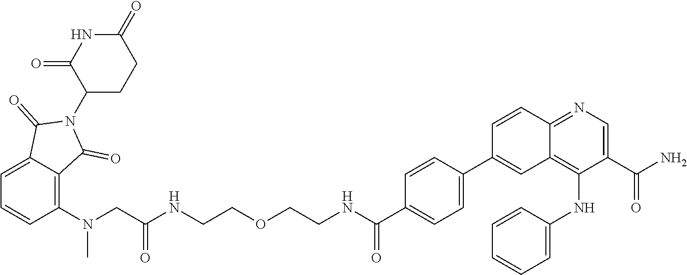 [SMILES: CN(CC(=O)NCCOCCNC(=O)c1ccc(-c2ccc3ncc(C(N)=O)c(Nc4ccccc4)c3c2)cc1)c1cccc2c1C(=O)N(C1CCC(=O)NC1=O)C2=O] |
| | 6-(4-((2-(2-(2-((2-(2,6-dioxopiperidin-3-yl)-1,3-dioxoisoindolin-4- |
| | yl)(methyl)amino)acetamido)ethoxy)ethyl)carbamoyl)phenyl)-4- |
| | (phenylamino)quinoline-3-carboxamide |
| 312 | 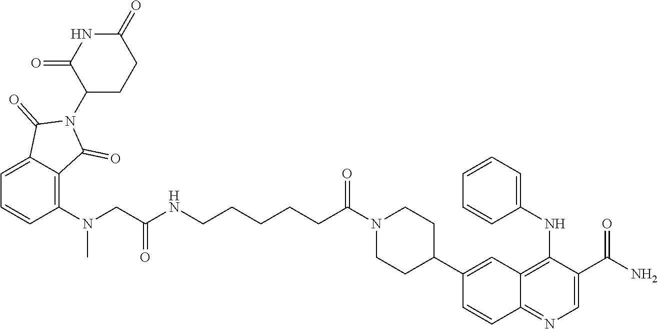 [SMILES: CN(CC(=O)NCCCCCC(=O)N1CCC(c2ccc3ncc(C(N)=O)c(Nc4ccccc4)c3c2)CC1)c1cccc2c1C(=O)N(C1CCC(=O)NC1=O)C2=O] |
| | 6-(1-(6-(2-((2-(2,6-dioxopiperidin-3-yl)-1,3-dioxoisoindolin-4- |
| | yl)(methyl)amino)acetamido)hexanoyl)piperidin-4-yl)-4- |
| | (phenylamino)quinoline-3-carboxamide |
| 340 | 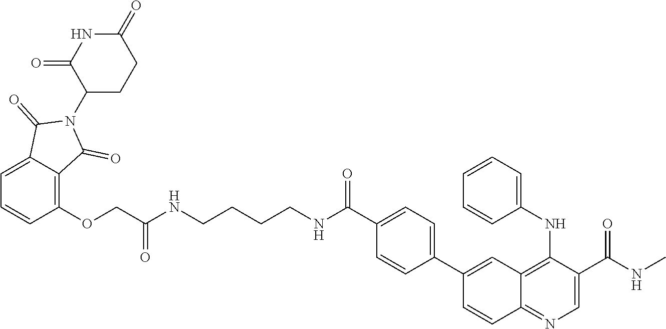 [SMILES: CNC(=O)c1cnc2ccc(-c3ccc(C(=O)NCCCCNC(=O)COc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)oxy)acetamido)butyl)carbamoyl)phenyl)-N-methyl-4- |
| | (phenylamino)quinoline-3-carboxamide |
| 341 | 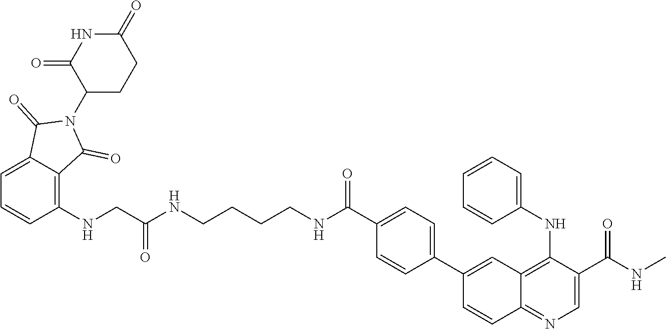 [SMILES: CNC(=O)c1cnc2ccc(-c3ccc(C(=O)NCCCCNC(=O)CNc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)amino)acetamido)butyl)carbamoyl)phenyl)-N-methyl-4- |
| | (phenylamino)quinoline-3-carboxamide |
| 343 | 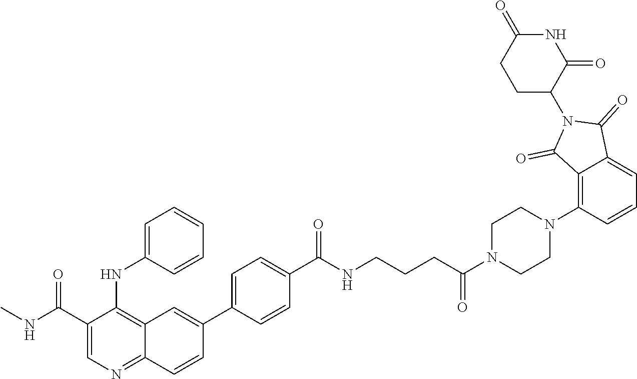 [SMILES: CNC(=O)c1cnc2ccc(-c3ccc(C(=O)NCCCC(=O)N4CCN(c5cccc6c5C(=O)N(C5CCC(=O)NC5=O)C6=O)CC4)cc3)cc2c1Nc1ccccc1] |
| | 6-(4-((4-(4-(2-(2,6-dioxopiperidin-3-yl)-1,3-dioxoisoindolin-4- |
| | yl)piperazin-1-yl)-4-oxobutyl)carbamoyl)phenyl)-N-methyl-4- |
| | (phenylamino)quinoline-3-carboxamide |
| 344 | 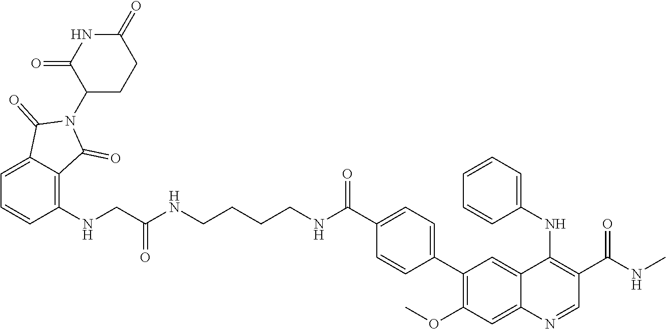 [SMILES: CNC(=O)c1cnc2cc(OC)c(-c3ccc(C(=O)NCCCCNC(=O)CNc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)amino)acetamido)butyl)carbamoyl)phenyl)-7-methoxy-N-methyl-4- |
| | (phenylamino)quinoline-3-carboxamide |
| 345 | 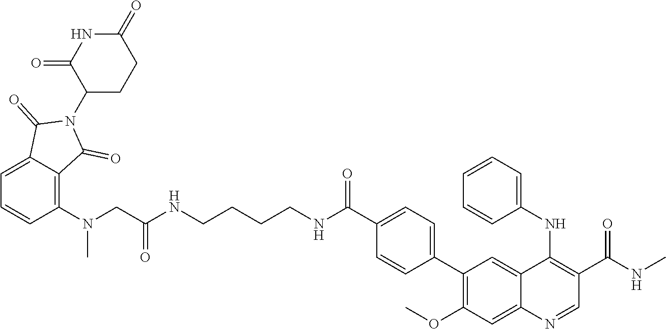 [SMILES: CNC(=O)c1cnc2cc(OC)c(-c3ccc(C(=O)NCCCCNC(=O)CN(C)c4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)(methyl)amino)acetamido)butyl)carbamoyl)phenyl)-7-methoxy-N- |
| | methyl-4-(phenylamino)quinoline-3-carboxamide |
| 346 | 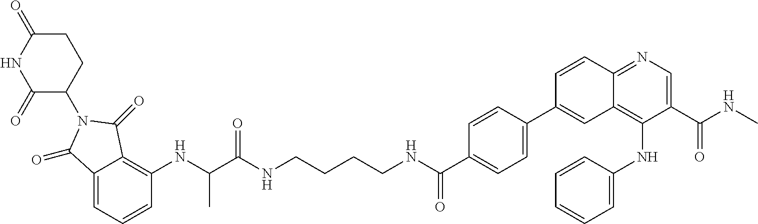 [SMILES: CNC(=O)c1cnc2ccc(-c3ccc(C(=O)NCCCCNC(=O)C(C)Nc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)amino)propanamido)butyl)carbamoyl)phenyl)-N-methyl-4- |
| | (phenylamino)quinoline-3-carboxamide |
| 347 | 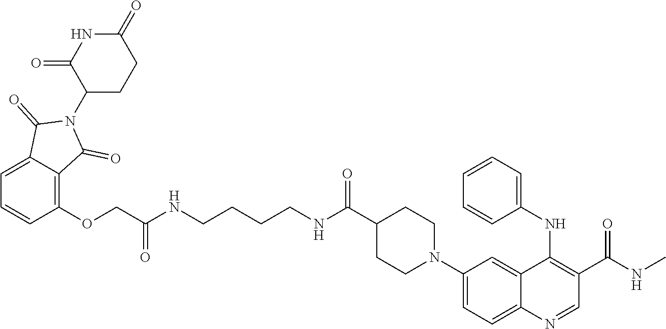 [SMILES: CNC(=O)c1cnc2ccc(N3CCC(C(=O)NCCCCNC(=O)COc4cccc5c4C(=O)N(C4CCC(=O)NC4=O)C5=O)CC3)cc2c1Nc1ccccc1] |
| | 6-(4-((4-(2-((2-(2,6-dioxopiperidin-3-yl)-1,3-dioxoisoindolin-4- |
| | yl)oxy)acetamido)butyl)carbamoyl)piperidin-1-yl)-N-methyl-4- |
| | (phenylamino)quinoline-3-carboxamide |
| 348 | 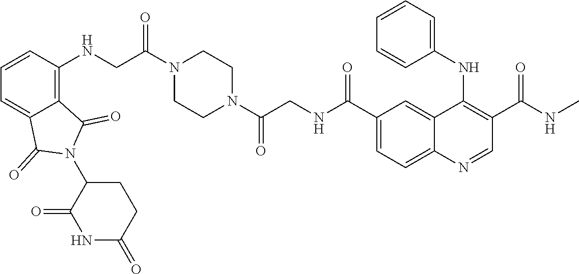 [SMILES: CNC(=O)c1cnc2ccc(C(=O)NCC(=O)N3CCN(C(=O)CNc4cccc5c4C(=O)N(C4CCC(=O)NC4=O)C5=O)CC3)cc2c1Nc1ccccc1] |
| | N6-(2-(4-((2-(2,6-dioxopiperidin-3-yl)-1,3-dioxoisoindolin-4- |
| | yl)glycyl)piperazin-1-yl)-2-oxoethyl)-N3-methyl-4- |
| | (phenylamino)quinoline-3,6-dicarboxamide |
| 350 | 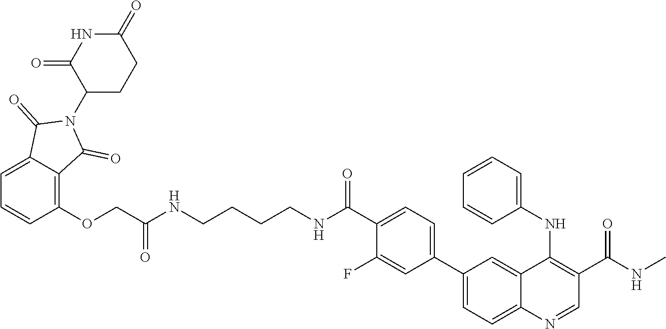 [SMILES: CNC(=O)c1cnc2ccc(-c3ccc(C(=O)NCCCCNC(=O)COc4cccc5c4C(=O)N(C4CCC(=O)NC4=O)C5=O)c(F)c3)cc2c1Nc1ccccc1] |
| | 6-(4-((4-(2-((2-(2,6-dioxopiperidin-3-yl)-1,3-dioxoisoindolin-4- |
| | yl)amino)acetamido)butyl)carbamoyl)-3-fluorophenyl)-N-methyl-4- |
| | (phenylamino)quinoline-3-carboxamide |
| 351 | 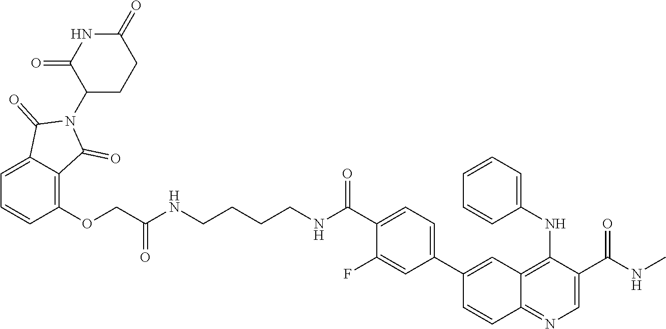 [SMILES: CNC(=O)c1cnc2ccc(-c3ccc(C(=O)NCCCCNC(=O)COc4cccc5c4C(=O)N(C4CCC(=O)NC4=O)C5=O)c(F)c3)cc2c1Nc1ccccc1] |
| | 6-(4-((4-(2-((2-(2,6-dioxopiperidin-3-yl)-1,3-dioxoisoindolin-4- |
| | yl)oxy)acetamido)butyl)carbamoyl)-3-fluorophenyl)-N-methyl-4- |
| | (phenylamino)quinoline-3-carboxamide |
| 353 | 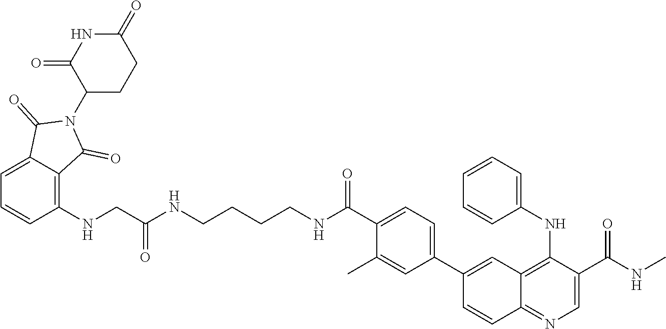 [SMILES: CNC(=O)c1cnc2ccc(-c3ccc(C(=O)NCCCCNC(=O)CNc4cccc5c4C(=O)N(C4CCC(=O)NC4=O)C5=O)c(C)c3)cc2c1Nc1ccccc1] |
| | 6-(4-((4-(2-((2-(2,6-dioxopiperidin-3-yl)-1,3-dioxoisoindolin-4- |
| | yl)amino)acetamido)butyl)carbamoyl)-3-methylphenyl)-N-methyl-4- |
| | (phenylamino)quinoline-3-carboxamide |
| 354 | 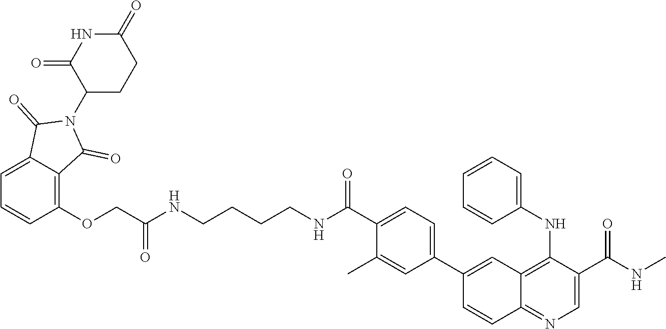 [SMILES: CNC(=O)c1cnc2ccc(-c3ccc(C(=O)NCCCCNC(=O)COc4cccc5c4C(=O)N(C4CCC(=O)NC4=O)C5=O)c(C)c3)cc2c1Nc1ccccc1] |
| | 6-(4-((4-(2-((2-(2,6-dioxopiperidin-3-yl)-1,3-dioxoisoindolin-4- |
| | yl)oxy)acetamido)butyl)carbamoyl)-3-methylphenyl)-N-methyl-4- |
| | (phenylamino)quinoline-3-carboxamide |
| 356 | 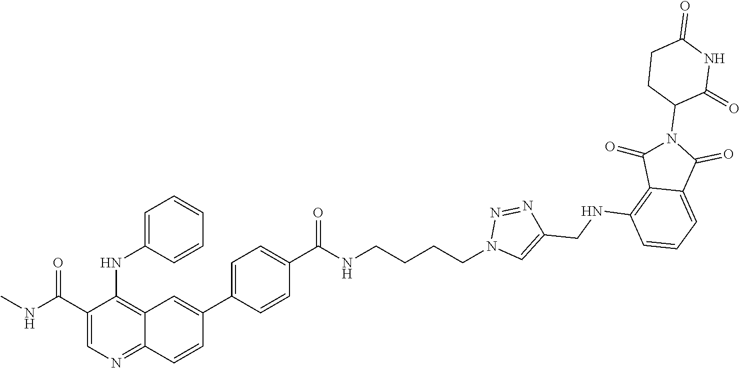 [SMILES: CNC(=O)c1cnc2ccc(-c3ccc(C(=O)NCCCCn4cc(CNc5cccc6c5C(=O)N(C5CCC(=O)NC5=O)C6=O)nn4)cc3)cc2c1Nc1ccccc1] |
| | 6-(4-((4-(4-(((2-(2,6-dioxopiperidin-3-yl)-1,3-dioxoisoindolin-4- |
| | yl)amino)methyl)-1H-1,2,3-triazol-1-yl)butyl)carbamoyl)phenyl)-N- |
| | methyl-4-(phenylamino)quinoline-3-carboxamide |
| 357 | 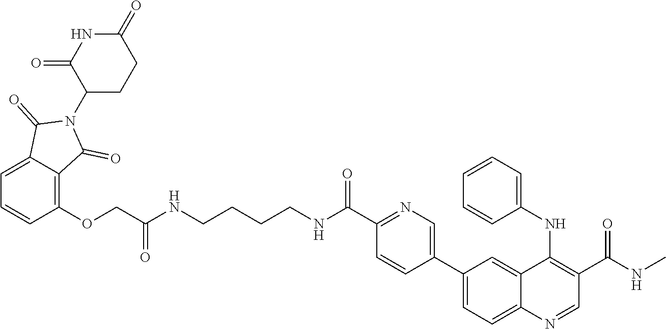 [SMILES: CNC(=O)c1cnc2ccc(-c3ccc(C(=O)NCCCCNC(=O)COc4cccc5c4C(=O)N(C4CCC(=O)NC4=O)C5=O)nc3)cc2c1Nc1ccccc1] |
| | 6-(6-((4-(2-((2-(2,6-dioxopiperidin-3-yl)-1,3-dioxoisoindolin-4- |
| | yl)oxy)acetamido)butyl)carbamoyl)pyridin-3-yl)-N-methyl-4- |
| | (phenylamino)quinoline-3-carboxamide |
| 358 | 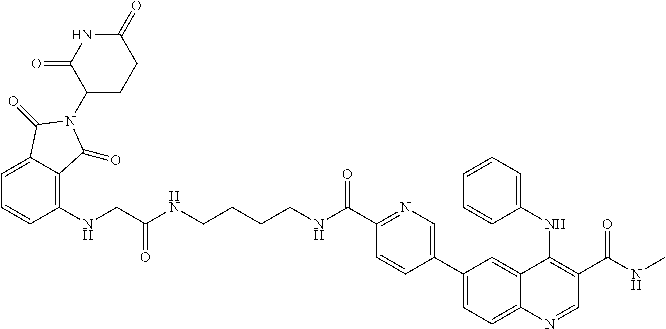 [SMILES: CNC(=O)c1cnc2ccc(-c3ccc(C(=O)NCCCCNC(=O)CNc4cccc5c4C(=O)N(C4CCC(=O)NC4=O)C5=O)nc3)cc2c1Nc1ccccc1] |
| | 6-(6-((4-(2-((2-(2,6-dioxopiperidin-3-yl)-1,3-dioxoisoindolin-4- |
| | yl)amino)acetamido)butyl)carbamoyl)pyridin-3-yl)-N-methyl-4- |
| | (phenylamino)quinoline-3-carboxamide |
| 359 | 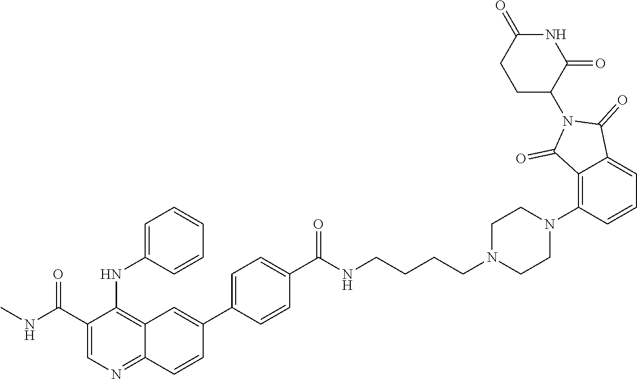 [SMILES: CNC(=O)c1cnc2ccc(-c3ccc(C(=O)NCCCCN4CCN(c5cccc6c5C(=O)N(C5CCC(=O)NC5=O)C6=O)CC4)cc3)cc2c1Nc1ccccc1] |
| | 6-(4-((4-(4-(2-(2,6-dioxopiperidin-3-yl)-1,3-dioxoisoindolin-4- |
| | yl)piperazin-1-yl)butyl)carbamoyl)phenyl)-N-methyl-4- |
| | (phenylamino)quinoline-3-carboxamide |
| 360 | 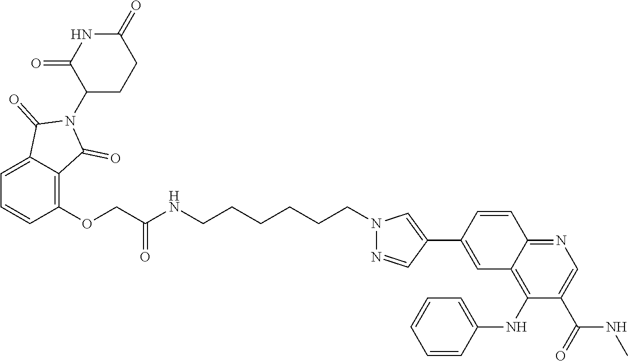 [SMILES: CNC(=O)c1cnc2ccc(-c3cnn(CCCCCCNC(=O)COc4cccc5c4C(=O)N(C4CCC(=O)NC4=O)C5=O)c3)cc2c1Nc1ccccc1] |
| | 6-(1-(6-(2-((2-(2,6-dioxopiperidin-3-yl)-1,3-dioxoisoindolin-4- |
| | yl)oxy)acetamido)hexyl)-1H-pyrazol-4-yl)-N-methyl-4- |
| | (phenylamino)quinoline-3-carboxamide |
| 361 | 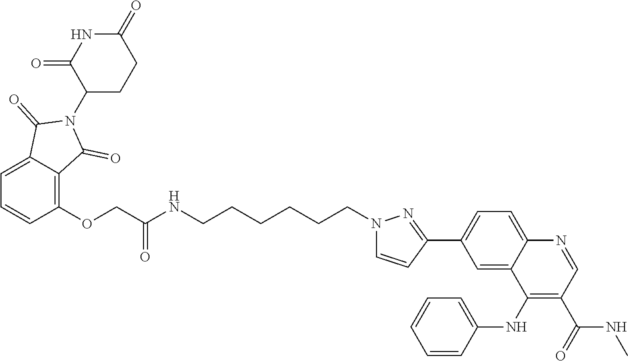 [SMILES: CNC(=O)c1cnc2ccc(-c3ccn(CCCCCCNC(=O)COc4cccc5c4C(=O)N(C4CCC(=O)NC4=O)C5=O)n3)cc2c1Nc1ccccc1] |
| | 6-(1-(6-(2-((2-(2,6-dioxopiperidin-3-yl)-1,3-dioxoisoindolin-4- |
| | yl)oxy)acetamido)hexyl)-1H-pyrazol-3-yl)-N-methyl-4- |
| | (phenylamino)quinoline-3-carboxamide |
| 363 | 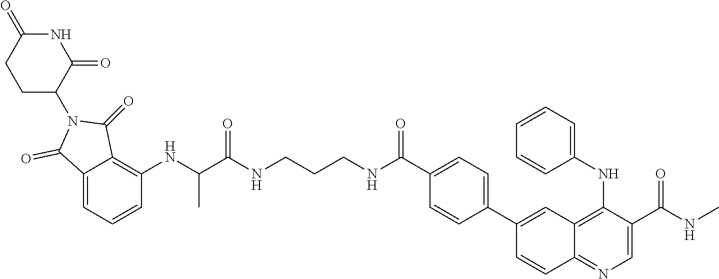 [SMILES: CNC(=O)c1cnc2ccc(-c3ccc(C(=O)NCCCNC(=O)C(C)Nc4cccc5c4C(=O)N(C4CCC(=O)NC4=O)C5=O)cc3)cc2c1Nc1ccccc1] |
| | 6-(4-((3-(2-((2-(2,6-dioxopiperidin-3-yl)-1,3-dioxoisoindolin-4- |
| | yl)amino)propanamido)propyl)carbamoyl)phenyl)-N-methyl-4- |
| | (phenylamino)quinoline-3-carboxamide |
| 365 | 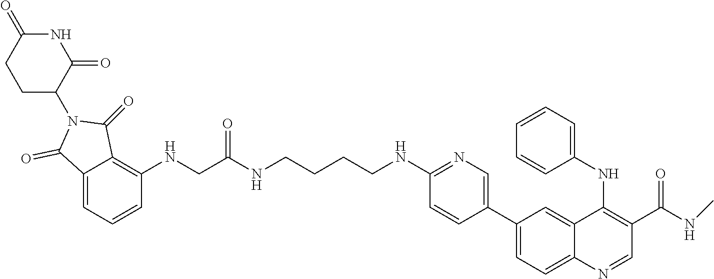 [SMILES: CNC(=O)c1cnc2ccc(-c3ccc(NCCCCNC(=O)CNc4cccc5c4C(=O)N(C4CCC(=O)NC4=O)C5=O)nc3)cc2c1Nc1ccccc1] |
| | 6-(6-((4-(2-((2-(2,6-dioxopiperidin-3-yl)-1,3-dioxoisoindolin-4- |
| | yl)amino)acetamido)butyl)amino)pyridin-3-yl)-N-methyl-4- |
| | (phenylamino)quinoline-3-carboxamide |
| 366 | 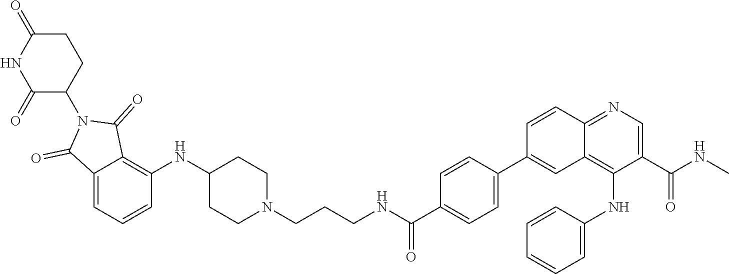 [SMILES: CNC(=O)c1cnc2ccc(-c3ccc(C(=O)NCCCN4CCC(Nc5cccc6c5C(=O)N(C5CCC(=O)NC5=O)C6=O)CC4)cc3)cc2c1Nc1ccccc1] |
| | 6-(4-((3-(4-((2-(2,6-dioxopiperidin-3-yl)-1,3-dioxoisoindolin-4- |
| | yl)amino)piperidin-1-yl)propyl)carbamoyl)phenyl)-N-methyl-4- |
| | (phenylamino)quinoline-3-carboxamide |
| 367 | 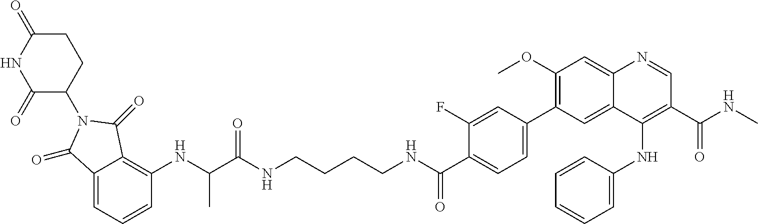 [SMILES: CNC(=O)c1cnc2cc(OC)c(-c3ccc(C(=O)NCCCCNC(=O)C(C)Nc4cccc5c4C(=O)N(C4CCC(=O)NC4=O)C5=O)c(F)c3)cc2c1Nc1ccccc1] |
| | 6-(4-((4-(2-((2-(2,6-dioxopiperidin-3-yl)-1,3-dioxoisoindolin-4- |
| | yl)amino)propanamido)butyl)carbamoyl)-3-fluorophenyl)-7-methoxy-N- |
| | methyl-4-(phenylamino)quinoline-3-carboxamide |
| 369 | 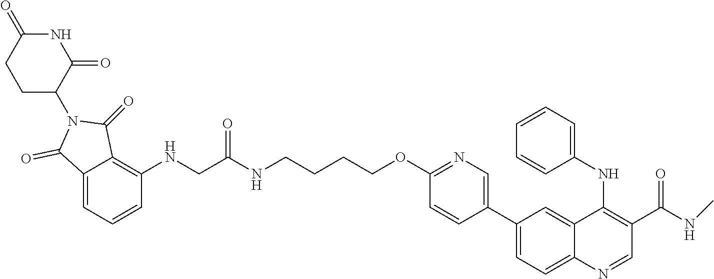 [SMILES: CNC(=O)c1cnc2ccc(-c3ccc(OCCCCNC(=O)CNc4cccc5c4C(=O)N(C4CCC(=O)NC4=O)C5=O)nc3)cc2c1Nc1ccccc1] |
| | 6-(6-(4-(2-((2-(2,6-dioxopiperidin-3-yl)-1,3-dioxoisoindolin-4- |
| | yl)amino)acetamido)butoxy)pyridin-3-yl)-N-methy-4- |
| | (phenylamino)quinoline-3-carboxamide |
| 370 | 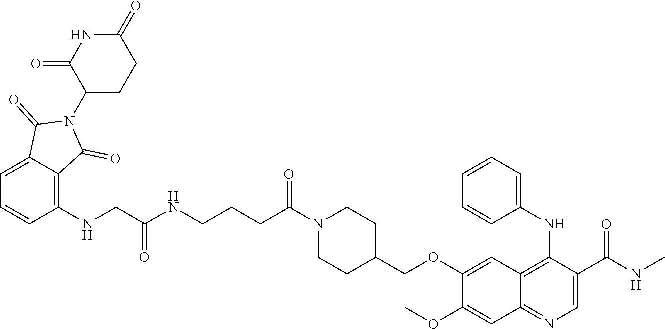 [SMILES: CNC(=O)c1cnc2cc(OC)c(OCC3CCN(C(=O)CCCNC(=O)CNc4cccc5c4C(=O)N(C4CCC(=O)NC4=O)C5=O)CC3)cc2c1Nc1ccccc1] |
| | 6-((1-(4-(2-((2-(2,6-dioxopiperidin-3-yl)-1,3-dioxoisoindolin-4- |
| | yl)amino)acetamido)butanoyl)piperidin-4-yl)methoxy)-7-methoxy-N- |
| | methyl-4-(phenylamino)quinoline-3-carboxamide |
| 371 | 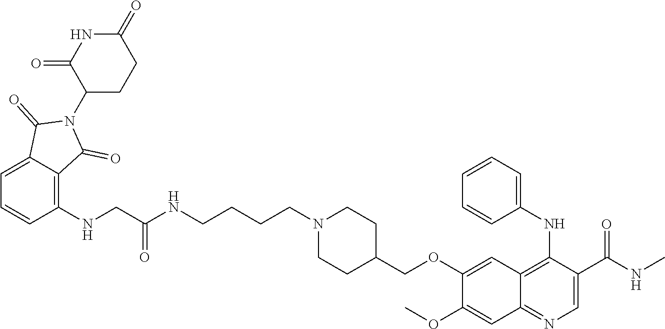 [SMILES: CNC(=O)c1cnc2cc(OC)c(OCC3CCN(CCCCNC(=O)CNc4cccc5c4C(=O)N(C4CCC(=O)NC4=O)C5=O)CC3)cc2c1Nc1ccccc1] |
| | 6-((1-(4-(2-((2-(2,6-dioxopiperidin-3-yl)-1,3-dioxoisoindolin-4- |
| | yl)amino)acetamido)butyl)piperidin-4-yl)methoxy)-7-methoxy-N- |
| | methyl-4-(phenylamino)quinoline-3-carboxamide |
| 372 | 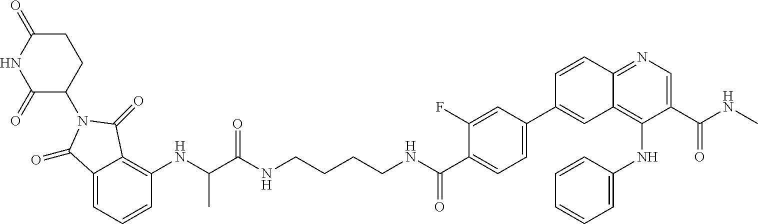 [SMILES: CNC(=O)c1cnc2ccc(-c3ccc(C(=O)NCCCCNC(=O)C(C)Nc4cccc5c4C(=O)N(C4CCC(=O)NC4=O)C5=O)c(F)c3)cc2c1Nc1ccccc1] |
| | 6-(4-((4-(2-((2-(2,6-dioxopiperidin-3-yl)-1,3-dioxoisoindolin-4- |
| | yl)amino)propanamido)butyl)carbamoyl)-3-fluorophenyl)-N-methyl-4- |
| | (phenylamino)quinoline-3-carboxamide |
| 373 | 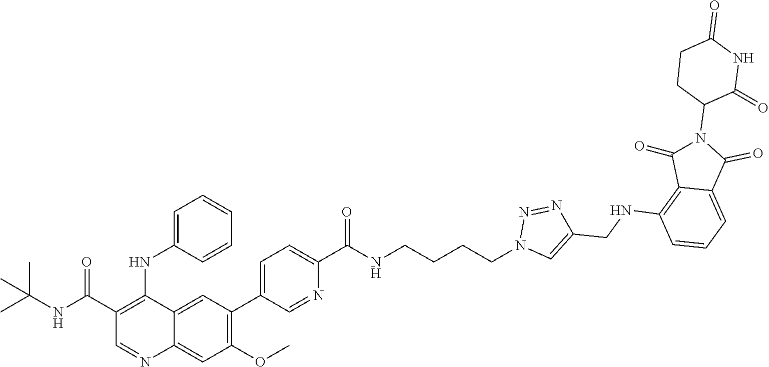 [SMILES: COc1cc2ncc(C(=O)NC(C)(C)C)c(Nc3ccccc3)c2cc1-c1ccc(C(=O)NCCCCn2cc(CNc3cccc4c3C(=O)N(C3CCC(=O)NC3=O)C4=O)nn2)nc1] |
| | N-(tert-butyl)-6-(6-((4-(4-(((2-(2,6-dioxopiperidin-3-yl)-1,3- |
| | dioxoisoindolin-4-yl)amino)methyl)-1H-1,2,3-triazol-1- |
| | yl)butyl)carbamooyl)pyridin-3-yl)-7-methoxy-4-(phenylamino)quinoline- |
| | 3-carboxamide |
| 374 | 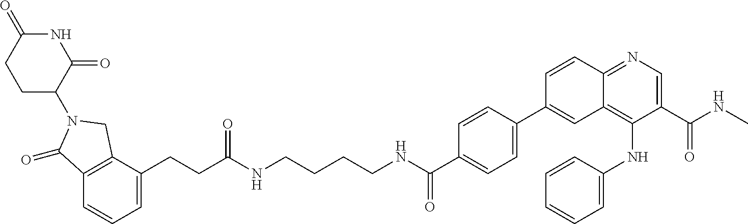 [SMILES: CNC(=O)c1cnc2ccc(-c3ccc(C(=O)NCCCCNC(=O)CCc4cccc5c4CN(C4CCC(=O)NC4=O)C5=O)cc3)cc2c1Nc1ccccc1] |
| | 6-(4-((4-(3-(2-(2,6-dioxopiperidin-3-yl)-1-oxoisoindolin-4- |
| | yl)propanamido)butyl)carbamoyl)phenyl)-N-methyl-4- |
| | (phenylamino)quinoline-3-carboxamide |
| 375 | 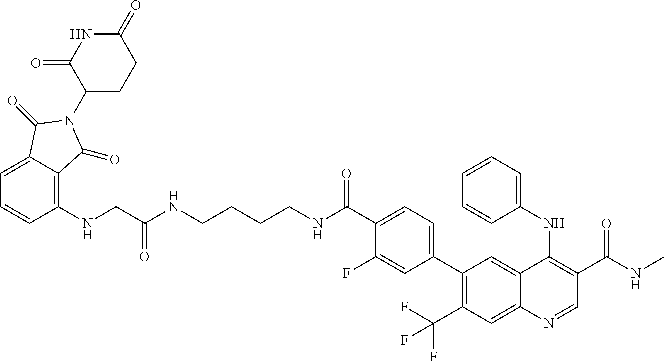 [SMILES: CNC(=O)c1cnc2cc(C(F)(F)F)c(-c3ccc(C(=O)NCCCCNC(=O)CNc4cccc5c4C(=O)N(C4CCC(=O)NC4=O)C5=O)c(F)c3)cc2c1Nc1ccccc1] |
| | 6-(4-((4-(2-((2-(2,6-dioxopiperidin-3-yl)-1,3-dioxoisoindolin-4- |
| | yl)amino)acetamido)butyl)carbamoyl)-3-fluorophenyl)-N-methyl-4- |
| | (phenylamino)-7-(trifluoromethyl)quinoline-3-carboxamide |
| 376 | 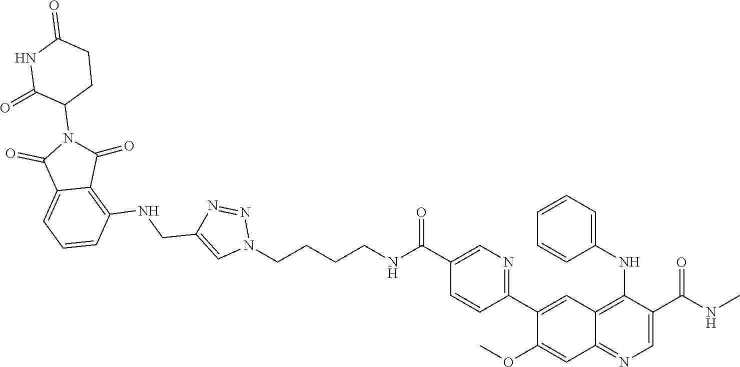 [SMILES: CNC(=O)c1cnc2cc(OC)c(-c3ccc(C(=O)NCCCCn4cc(CNc5cccc6c5C(=O)N(C5CCC(=O)NC5=O)C6=O)nn4)cn3)cc2c1Nc1ccccc1] |
| | 6-(4-((2-(4-((2-(2,6-dioxopiperidin-3-yl)-1,3-dioxoisoindolin-4- |
| | yl)amino)piperidin-1-yl)ethyl)carbamoyl)phenyl)-N-methyl-4- |
| | (phenylamino)quinoline-3-carboxamide |
| 378 | 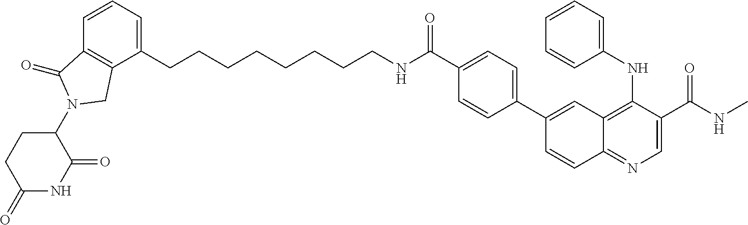 [SMILES: CNC(=O)c1cnc2ccc(-c3ccc(C(=O)NCCCCCCCCc4cccc5c4CN(C4CCC(=O)NC4=O)C5=O)cc3)cc2c1Nc1ccccc1] |
| | 6-(4-((8-(2-(2,6-dioxopiperidin-3-yl)-1-oxoisoindolin-4- |
| | yl)octyl)carbamoyl)phenyl)-N-methyl-4-(phenylamino)quinoline-3- |
| | carboxamide |
| 379 | 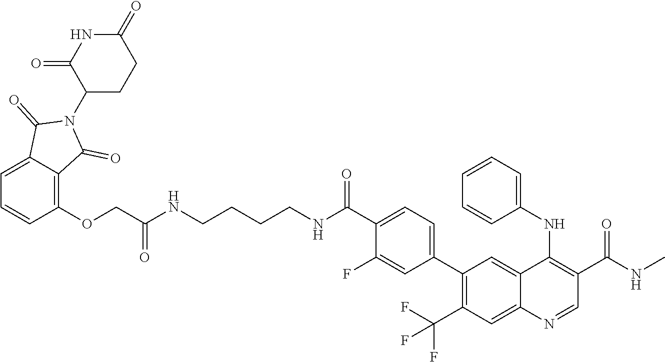 [SMILES: CNC(=O)c1cnc2cc(C(F)(F)F)c(-c3ccc(C(=O)NCCCCNC(=O)COc4cccc5c4C(=O)N(C4CCC(=O)NC4=O)C5=O)c(F)c3)cc2c1Nc1ccccc1] |
| | 6-(4-((4-(2-((2-(2,6-dioxopiperidin-3-yl)-1,3-dioxoisoindolin-4- |
| | yl)oxy)acetamido)butyl)carbamoyl)-3-fluorophenyl)-N-methyl-4- |
| | (phenylamino)-7-(trifluoromethyl)quinoline-3-carboxamide |
| 380 | 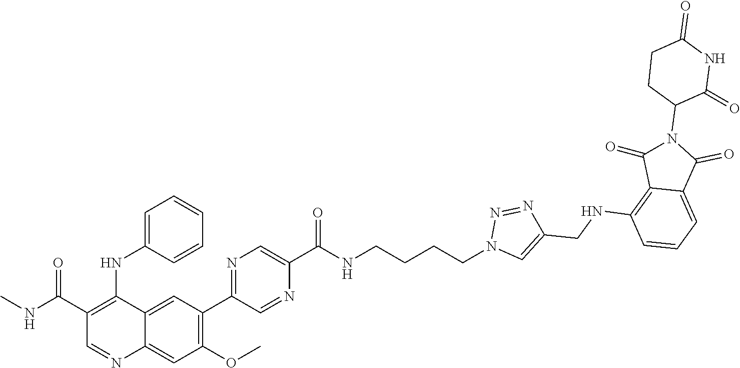 [SMILES: CNC(=O)c1cnc2cc(OC)c(-c3cnc(C(=O)NCCCCn4cc(CNc5cccc6c5C(=O)N(C5CCC(=O)NC5=O)C6=O)nn4)cn3)cc2c1Nc1ccccc1] |
| | 6-(5-((4-(4-(((2-(2,6-dioxopiperidin-3-yl)-1,3-dioxoisoindolin-4- |
| | yl)amino)methyl)-1H-1,2,3-triazol-1-yl)butyl)carbamoyl)pyrazin-2-yl)- |
| | 7-methoxy-N-methyl-4-(phenylamino)quinoline-3-carboxamide |
| 381 | 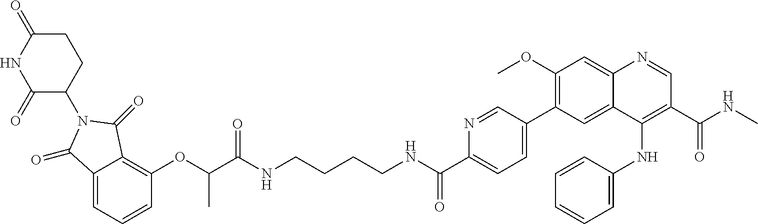 [SMILES: CNC(=O)c1cnc2cc(OC)c(-c3ccc(C(=O)NCCCCNC(=O)C(C)Oc4cccc5c4C(=O)N(C4CCC(=O)NC4=O)C5=O)nc3)cc2c1Nc1ccccc1] |
| | 6-(6-((4-(2-((2-(2,6-dioxopiperidin-3-yl)-1,3-dioxoisoindolin-4- |
| | yl)oxy)propanamido)butyl)carbamoyl)pyridin-3-yl)-7-methoxy-N- |
| | methyl-4-(phenylamino)quinoline-3-carboxamide |
| 382 | 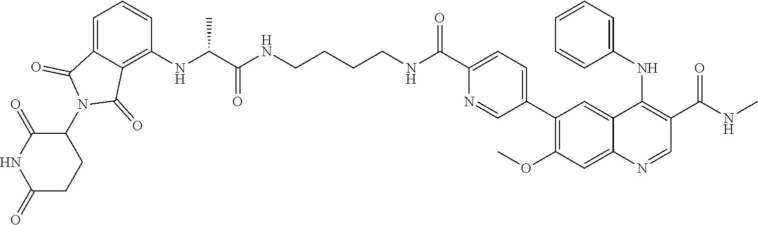 [SMILES: CNC(=O)c1cnc2cc(OC)c(-c3ccc(C(=O)NCCCCNC(=O)[C@@H](C)Nc4cccc5c4C(=O)N(C4CCC(=O)NC4=O)C5=O)nc3)cc2c1Nc1ccccc1] |
| | 6-(6-((4-((2R)-2-((2-(2,6-dioxopiperidin-3-yl)-1,3-dioxoisoindolin-4- |
| | yl)amino)propanamido)butyl)carbamoyl)pyridin-3-yl)-7-methoxy-N- |
| | methyl-4-(phenylamino)quinoline-3-carboxamide |
| 384 | 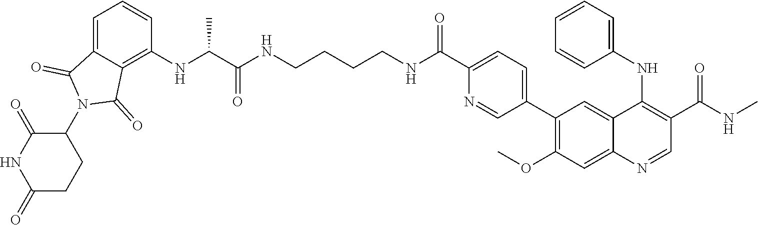 [SMILES: CNC(=O)c1cnc2cc(OC)c(-c3ccc(C(=O)NCCCCNC(=O)[C@@H](C)Nc4cccc5c4C(=O)N(C4CCC(=O)NC4=O)C5=O)nc3)cc2c1Nc1ccccc1] |
| | 6-(6-((4-((2R)-2-((2-(2,6-dioxopiperidin-3-yl)-1,3-dioxoisoindolin-4- |
| | yl)amino)propanamido)butyl)carbamoyl)pyridin-3-yl)-7-methoxy-N- |
| | methyl-4-(phenylamino)quinoline-3-carboxamide |
| 385 | 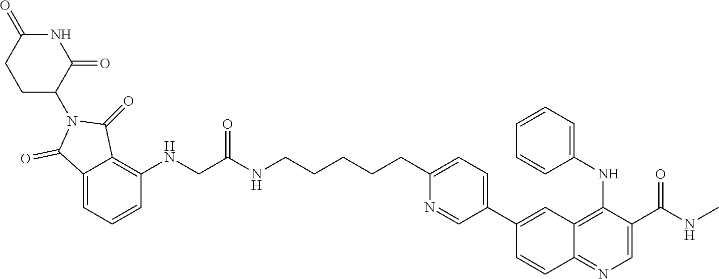 [SMILES: CNC(=O)c1cnc2ccc(-c3ccc(CCCCCNC(=O)CNc4cccc5c4C(=O)N(C4CCC(=O)NC4=O)C5=O)nc3)cc2c1Nc1ccccc1] |
| | 6-(6-(5-(2-((2-(2,6-dioxopiperidin-3-yl)-1,3-dioxoisoindolin-4- |
| | yl)amino)acetamido)pentyl)pyridin-3-yl)-N-methyl-4- |
| | (phenylamino)quinoline-3-carboxamide |
| 386 | 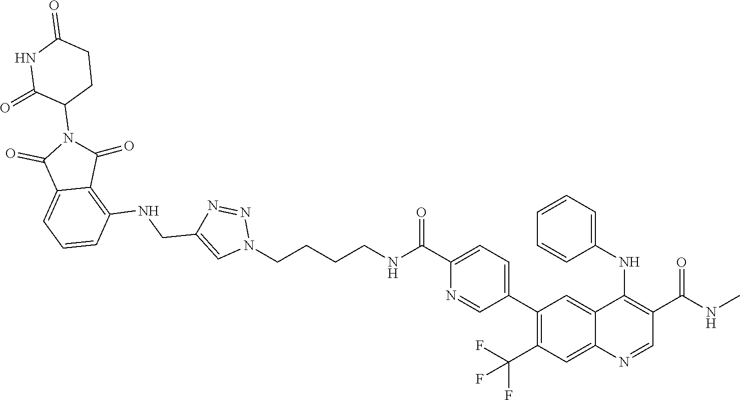 [SMILES: CNC(=O)c1cnc2cc(C(F)(F)F)c(-c3ccc(C(=O)NCCCCn4cc(CNc5cccc6c5C(=O)N(C5CCC(=O)NC5=O)C6=O)nn4)nc3)cc2c1Nc1ccccc1] |
| | 6-(6-((4-(4-(((2-(2,6-dioxopiperidin-3-yl)-1,3-dioxoisoindolin-4- |
| | yl)amino)methyl)-1H-1,2,3-triazol-1-yl)butyl)carbamoyl)pyridin-3-yl)- |
| | N-methyl-4-(phenylamino)-7-(trifluoromethyl)quinoline-3-carboxamide |
| 387 | 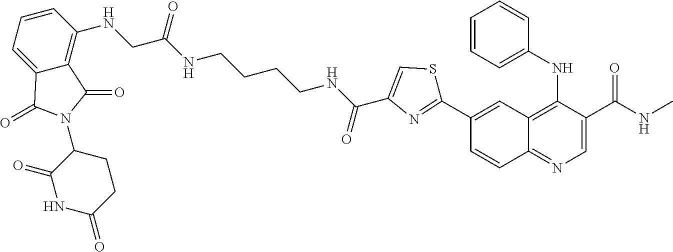 [SMILES: CNC(=O)c1cnc2ccc(-c3nc(C(=O)NCCCCNC(=O)CNc4cccc5c4C(=O)N(C4CCC(=O)NC4=O)C5=O)cs3)cc2c1Nc1ccccc1] |
| | N-(4-(2-((2-(2,6-dioxopiperidin-3-yl)-1,3-dioxoisoindolin-4- |
| | yl)amino)acetamido)butyl)-2-(3-(methylcarbamoyl)-4- |
| | (phenylamino)quinolin-6-yl)thiazole-4-carboxamide |
| 388 | 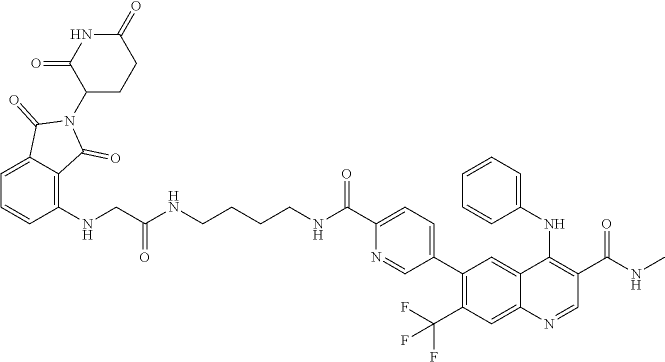 [SMILES: CNC(=O)c1cnc2cc(C(F)(F)F)c(-c3ccc(C(=O)NCCCCNC(=O)CNc4cccc5c4C(=O)N(C4CCC(=O)NC4=O)C5=O)nc3)cc2c1Nc1ccccc1] |
| | 6-(6-((4-(2-((2-(2,6-dioxopiperidin-3-yl)-1,3-dioxoisoindolin-4- |
| | yl)amino)acetamido)butyl)carbamoyl)pyridin-3-yl)-N-methyl-4- |
| | (phenylamino)-7-(trifluoromethyl)quinoline-3-carboxamide |
| 389 | 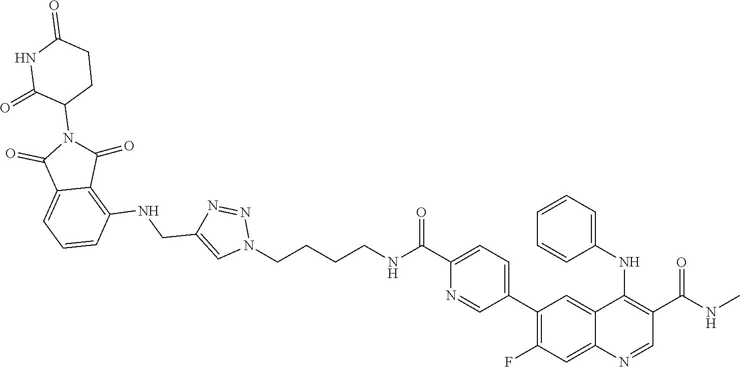 [SMILES: CNC(=O)c1cnc2cc(F)c(-c3ccc(C(=O)NCCCCn4cc(CNc5cccc6c5C(=O)N(C5CCC(=O)NC5=O)C6=O)nn4)nc3)cc2c1Nc1ccccc1] |
| | 6-(6-((4-(4-(((2-(2,6-dioxopiperidin-3-yl)-1,3-dioxoisoindolin-4- |
| | yl)amino)methyl)-1H-1,2,3-triazol-1-yl)butyl)carbamoyl)pyridin-3-yl)-7- |
| | fluoro-N-methyl-4-(phenylamino)quinoline-3-carboxamide |
| 395 | 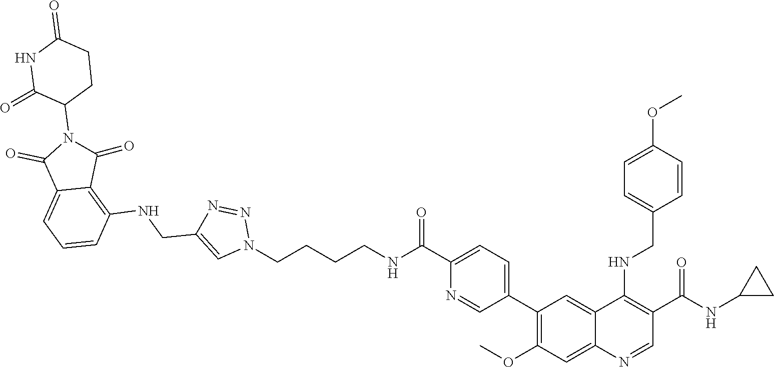 [SMILES: COc1ccc(CNc2c(C(=O)NC3CC3)cnc3cc(OC)c(-c4ccc(C(=O)NCCCCn5cc(CNc6cccc7c6C(=O)N(C6CCC(=O)NC6=O)C7=O)nn5)nc4)cc23)cc1] |
| | N-cyclopropyl-6-(6-((4-(4-(((2-(2,6-dioxopiperidin-3-yl)-1,3- |
| | dioxoisoindolin-4-yl)amino)methyl)-1H-1,2,3-triazol-1- |
| | yl)butyl)carbamoyl)pyridin-3-yl)-7-methoxy-4-((4- |
| | methoxybenzyl)amino)quinoline-3-carboxamide |
| 396 | 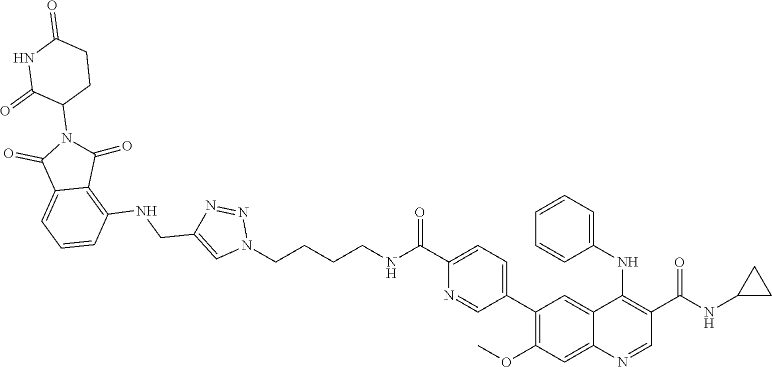 [SMILES: COc1cc2ncc(C(=O)NC3CC3)c(Nc3ccccc3)c2cc1-c1ccc(C(=O)NCCCCn2cc(CNc3cccc4c3C(=O)N(C3CCC(=O)NC3=O)C4=O)nn2)nc1] |
| | N-cyclopropyl-6-(6-((4-(4-(((2-(2,6-dioxopiperidin-3-yl)-1,3- |
| | dioxoisoindolin-4-yl)amino)methyl)-1H-1,2,3-triazol-1- |
| | yl)butyl)carbamoyl)pyridin-3-yl)-7-methoxy-4-(phenylamino)quinoline- |
| | 3-carboxamide |
| 397 | 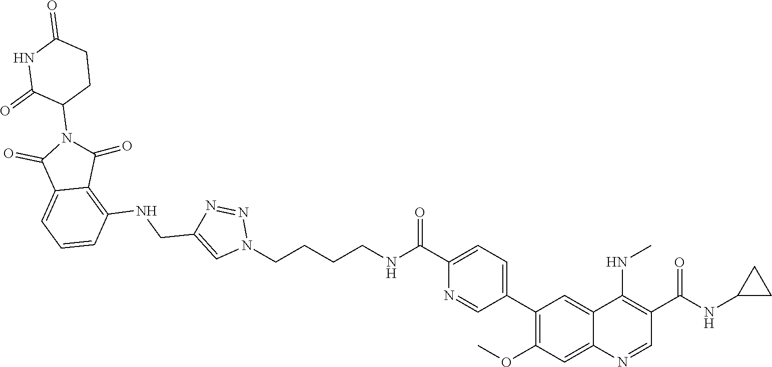 [SMILES: CNc1c(C(=O)NC2CC2)cnc2cc(OC)c(-c3ccc(C(=O)NCCCCn4cc(CNc5cccc6c5C(=O)N(C5CCC(=O)NC5=O)C6=O)nn4)nc3)cc12] |
| | N-cyclopropyl-6-(6-((4-(4-(((2-(2,6-dioxopiperidin-3-yl)-1,3- |
| | dioxoisoindolin-4-yl)amino)methyl)-1H-1,2,3-triazol-1- |
| | yl)butyl)carbamoyl)pyridin-3-yl)-7-methoxy-4-(methylamino)quinoline- |
| | 3-carboxamide |
| 398 | 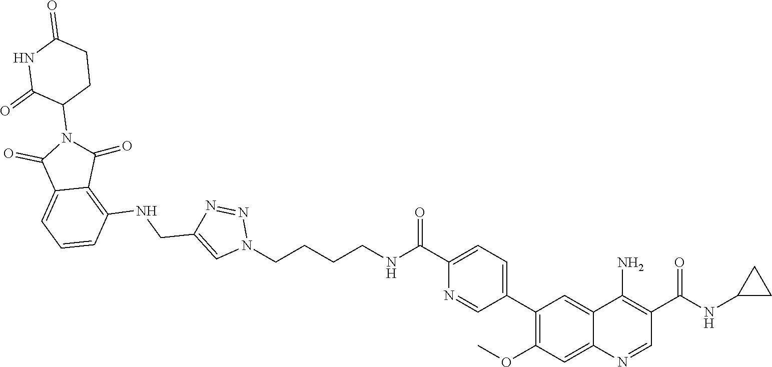 [SMILES: COc1cc2ncc(C(=O)NC3CC3)c(N)c2cc1-c1ccc(C(=O)NCCCCn2cc(CNc3cccc4c3C(=O)N(C3CCC(=O)NC3=O)C4=O)nn2)nc1] |
| | 4-amino-N-cyclopropyl-6-(6-((4-(4-(((2-(2,6-dioxopiperidin-3-yl)-1,3- |
| | dioxoisoindolin-4-yl)amino)methyl)-1H-1,2,3-triazol-1- |
| | yl)butyl)carbamoyl)pyridin-3-yl)-7-methoxyquinoline-3-carboxamide |
| 401 | 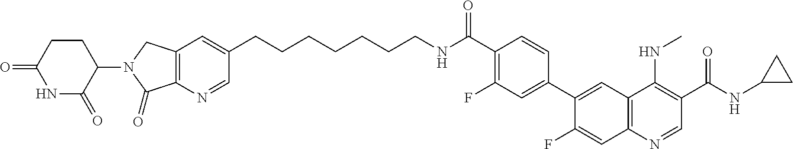 [SMILES: CNc1c(C(=O)NC2CC2)cnc2cc(F)c(-c3ccc(C(=O)NCCCCCCCc4cnc5c(c4)CN(C4CCC(=O)NC4=O)C5=O)c(F)c3)cc12] |
| | N-cyclopropyl-6-(4-((7-(6-(2,6-dioxopiperidin-3-yl)-7-oxo-6,7-dihydro- |
| | 5H-pyrrolo[3,4-b]pyridin-3-yl)heptyl)carbamoyl)-3-fluorophenyl)-7- |
| | fluoro-4-(methylamino)quinoline-3-carboxamide |
| 404 | 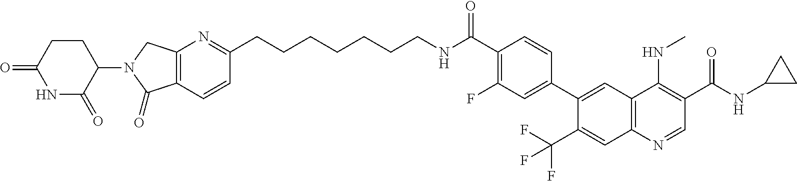 [SMILES: CNc1c(C(=O)NC2CC2)cnc2cc(C(F)(F)F)c(-c3ccc(C(=O)NCCCCCCCc4ccc5c(n4)CN(C4CCC(=O)NC4=O)C5=O)c(F)c3)cc12] |
| | N-cyclopropyl-6-(4-((7-(6-(2,6-dioxopiperidin-3-yl)-5-oxo-5,7-dihydro- |
| | 5H-pyrrolo[3,4-b]pyridin-2-yl)heptyl)carbamoyl)-3-fluorophenyl)-4- |
| | (methylamino)-7-(trifluoromethyl)quinoline-3-carboxamide |
| 405 | 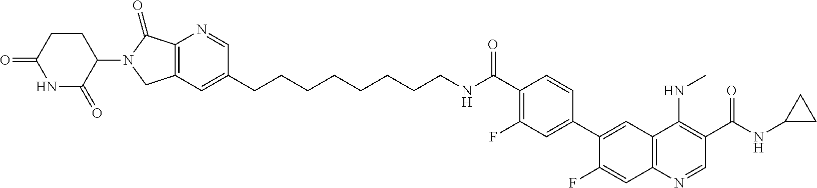 [SMILES: CNc1c(C(=O)NC2CC2)cnc2cc(F)c(-c3ccc(C(=O)NCCCCCCCCc4cnc5c(c4)CN(C4CCC(=O)NC4=O)C5=O)c(F)c3)cc12] |
| | N-cyclopropyl-6-(4-((8-(6-(2,6-dioxopiperidin-3-yl)-7-oxo-6,7-dihydro- |
| | 5H-pyrrolo[3,4-b]pyridin-3-yl)octyl)carbamoyl)-3-fluorophenyl)-7- |
| | fluoro-4-(methylamino)quinoline-3-carboxamide |
| 408 | 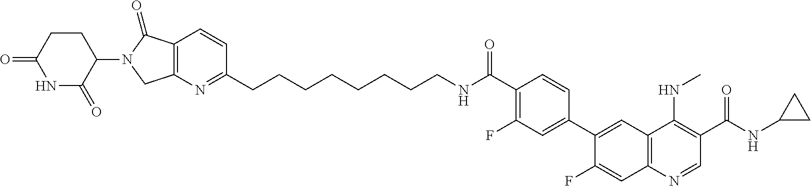 [SMILES: CNc1c(C(=O)NC2CC2)cnc2cc(F)c(-c3ccc(C(=O)NCCCCCCCCc4ccc5c(n4)CN(C4CCC(=O)NC4=O)C5=O)c(F)c3)cc12] |
| | N-cyclopropyl-6-(4-((8-(6-(2,6-dioxopiperidin-3-yl)-5-oxo-5,7-dihydro- |
| | 5H-pyrrolo[3,4-b]pyridin-2-yl)octyl)carbamoyl)-3-fluorophenyl)-7- |
| | fluoro-4-(methylamino)quinoline-3-carboxamide |
| 409 | 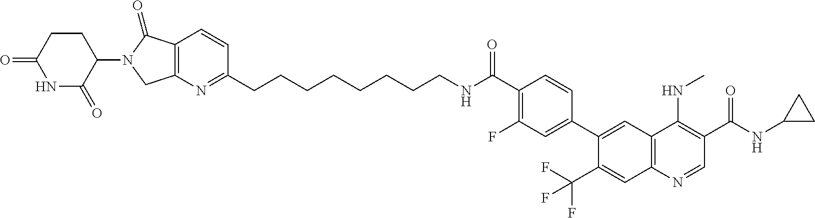 [SMILES: CNc1c(C(=O)NC2CC2)cnc2cc(C(F)(F)F)c(-c3ccc(C(=O)NCCCCCCCCc4ccc5c(n4)CN(C4CCC(=O)NC4=O)C5=O)c(F)c3)cc12] |
| | N-cyclopropyl-6-(4-((8-(6-(2,6-dioxopiperidin-3-yl)-5-oxo-6,7-dihydro- |
| | 5H-pyrrolo[3,4-b]pyridin-2-yl)octyl)carbamoyl)-3-fluorophenyl)-4- |
| | (methylamino)-7-(trifluoromethyl)quinoline-3-carboxamide |
| 411 | 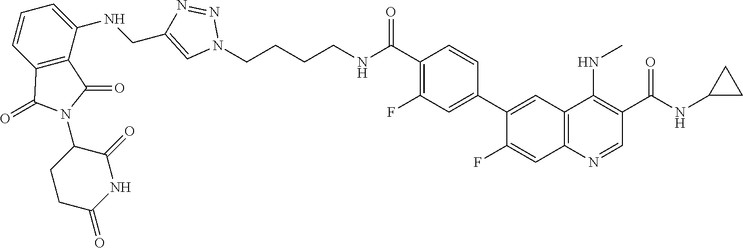 [SMILES: CNc1c(C(=O)NC2CC2)cnc2cc(F)c(-c3ccc(C(=O)NCCCCn4cc(CNc5cccc6c5C(=O)N(C5CCC(=O)NC5=O)C6=O)nn4)c(F)c3)cc12] |
| | N-cyclopropyl-6-(4-((4-(4-(((2-(2,6-dioxopiperidin-3-yl)-1,3- |
| | dioxoisoindolin-4-yl)amino)methyl)-1H-1,2,3-triazol-1- |
| | yl)butyl)carbamoyl)-3-fluorophenyl)-7-fluoro-4- |
| | (methylamino)quinoline-3-carboxamide |
| 412 | 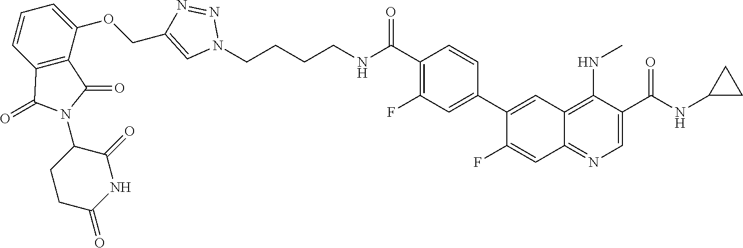 [SMILES: CNc1c(C(=O)NC2CC2)cnc2cc(F)c(-c3ccc(C(=O)NCCCCn4cc(COc5cccc6c5C(=O)N(C5CCC(=O)NC5=O)C6=O)nn4)c(F)c3)cc12] |
| | N-cyclopropyl-6-(4-((4-(4-(((2-(2,6-dioxopiperidin-3-yl)-1,3- |
| | dioxoisoindolin-4-yl)oxy)methyl)-1H-1,2,3-triazol-1- |
| | yl)butyl)carbamoyl)-3-fluorophenyl)-7-fluoro-4- |
| | (methylamino)quinoline-3-carboxamide |
| 413 | 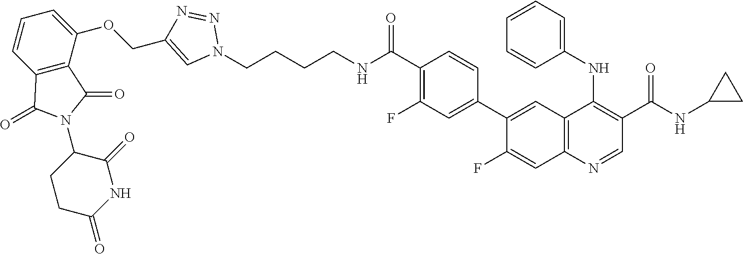 [SMILES: O=C1CCC(N2C(=O)c3cccc(OCc4cn(CCCCNC(=O)c5ccc(-c6cc7c(Nc8ccccc8)c(C(=O)NC8CC8)cnc7cc6F)cc5F)nn4)c3C2=O)C(=O)N1] |
| | N-cyclopropyl-6-(4-((4-(4-(((2-(2,6-dioxopiperidin-3-yl)-1,3- |
| | dioxoisoindolin-4-yl)oxy)methyl)-1H-1,2,3-triazol-1- |
| | yl)butyl)carbamoyl)-3-fluorophenyl)-7-fluoro-4- |
| | (phenylamino)quinoline-3-carboxamide |
| 414 | 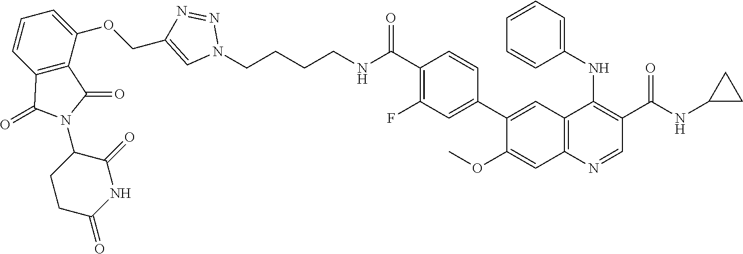 [SMILES: COc1cc2ncc(C(=O)NC3CC3)c(Nc3ccccc3)c2cc1-c1ccc(C(=O)NCCCCn2cc(COc3cccc4c3C(=O)N(C3CCC(=O)NC3=O)C4=O)nn2)c(F)c1] |
| | N-cyclopropyl-6-(4-((4-(4-(((2-(2,6-dioxopiperidin-3-yl)-1,3- |
| | dioxoisoindolin-4-yl)oxy)methyl)-1H-1,2,3-triazol-1- |
| | yl)butyl)carbamoyl)-3-fluorophenyl)-7-methoxy-4- |
| | (phenylamino)quinoline-3-carboxamide |
| 415 | 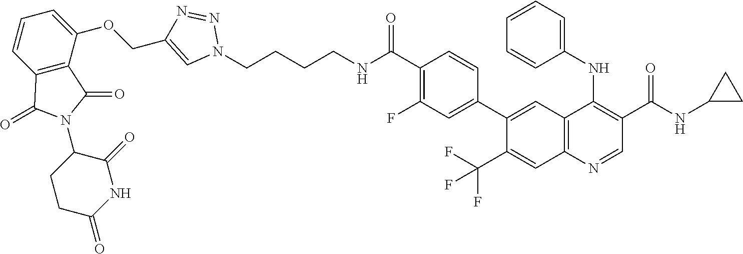 [SMILES: O=C1CCC(N2C(=O)c3cccc(OCc4cn(CCCCNC(=O)c5ccc(-c6cc7c(Nc8ccccc8)c(C(=O)NC8CC8)cnc7cc6C(F)(F)F)cc5F)nn4)c3C2=O)C(=O)N1] |
| | N-cyclopropyl-6-(4-((4-(4-(((2-(2,6-dioxopiperidin-3-yl)-1,3- |
| | dioxoisoindolin-4-yl)oxy)methyl)-1H-1,2,3-triazol-1- |
| | yl)butyl)carbamoyl)-3-fluorophenyl)-4-(phenylamino)-7- |
| | (trifluoromethyl)quinoline-3-carboxamide |
| 416 | 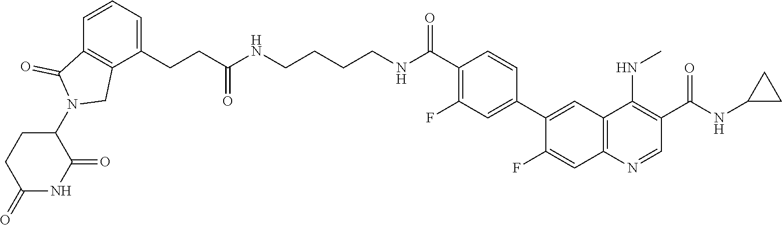 [SMILES: CNc1c(C(=O)NC2CC2)cnc2cc(F)c(-c3ccc(C(=O)NCCCCNC(=O)CCc4cccc5c4CN(C4CCC(=O)NC4=O)C5=O)c(F)c3)cc12] |
| | N-cyclopropyl-6-(4-((4-(3-(2-(2,6-dioxopiperidin-3-yl)-1-oxoisoindolin- |
| | 4-yl)propanamido)butyl)carbamoyl)-3-fluorophenyl)-7-fluoro-4- |
| | (methylamino)quinoline-3-carboxamide |
| 421 | 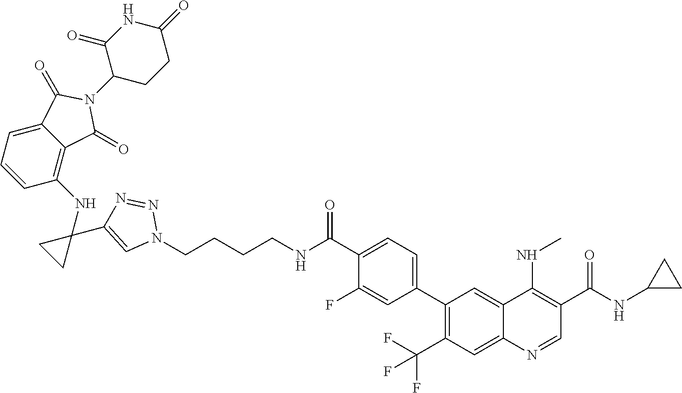 [SMILES: CNc1c(C(=O)NC2CC2)cnc2cc(C(F)(F)F)c(-c3ccc(C(=O)NCCCCn4cc(C5(Nc6cccc7c6C(=O)N(C6CCC(=O)NC6=O)C7=O)CC5)nn4)c(F)c3)cc12] |
| | N-cyclopropyl-6-(4-((4-(4-(1-((2-(2,6-dioxopiperidin-3-yl)-1,3- |
| | dioxoisoindolin-4-yl)amino)cyclopropyl)-1H-1,2,3-triazol-1- |
| | yl)butyl)carbamoyl)-3-fluorophenyl)-4-(methlylamino)-7- |
| | (trifluoromethyl)quinoline-3-carboxamide |
| 424 | 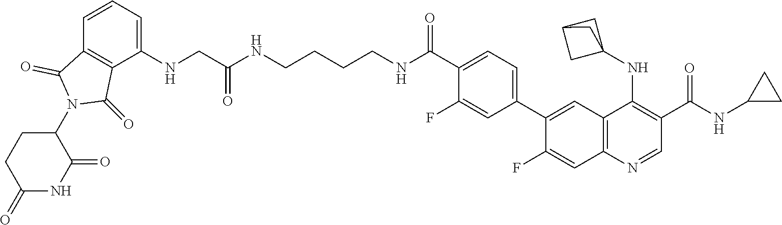 [SMILES: O=C(CNc1cccc2c1C(=O)N(C1CCC(=O)NC1=O)C2=O)NCCCCNC(=O)c1ccc(-c2cc3c(NC45CC(C4)C5)c(C(=O)NC4CC4)cnc3cc2F)cc1F] |
| | 4-(bicyclo[1.1.1]pentan-1-ylamino)-N-cyclopropyl-6-(4-((4-(2-((2-(2,6- |
| | dioxopiperidin-3-yl)-1,3-dioxoisoindolin-4- |
| | yl)amino)acetamido)butyl)carbamoyl)-3-fluorophenyl)-7- |
| | fluoroquinoline-3-carboxamide |
| 429 | 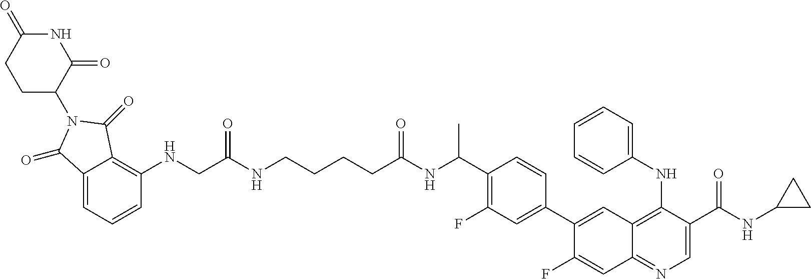 [SMILES: CC(NC(=O)CCCCNC(=O)CNc1cccc2c1C(=O)N(C1CCC(=O)NC1=O)C2=O)c1ccc(-c2cc3c(Nc4ccccc4)c(C(=O)NC4CC4)cnc3cc2F)cc1F] |
| | N-cyclopropyl-6-(4-(1-(5-(2-((2-(2,6-dioxopiperidin-3-yl)-1,3- |
| | dioxoisoindolin-4-yl)amino)acetamido)pentanamido)ethyl)-3- |
| | fluorophenyl)-7-fluoro-4-(phenylamino)quinoline-3-carboxamide |
| 430 | 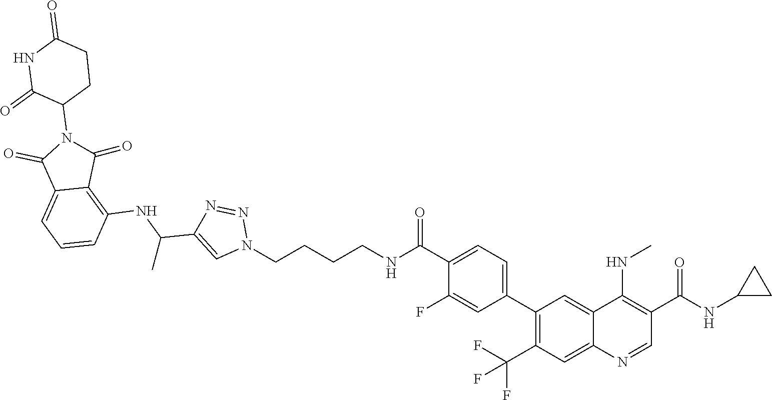 [SMILES: CNc1c(C(=O)NC2CC2)cnc2cc(C(F)(F)F)c(-c3ccc(C(=O)NCCCCn4cc(C(C)Nc5cccc6c5C(=O)N(C5CCC(=O)NC5=O)C6=O)nn4)c(F)c3)cc12] |
| | N-cyclopropyl-6-(4-((4-(4-(1-((2-(2,6-dioxopiperidin-3-yl)-1,3- |
| | dioxoisoindolin-4-yl)amino)ethyl)-1H-1,2,3-triazol-1- |
| | yl)butyl)carbamoyl)-3-fluorophenyl)-4-(methylamino)-7- |
| | (trifluoromethyl)quinoline-3-carboxamide |
| 431 | 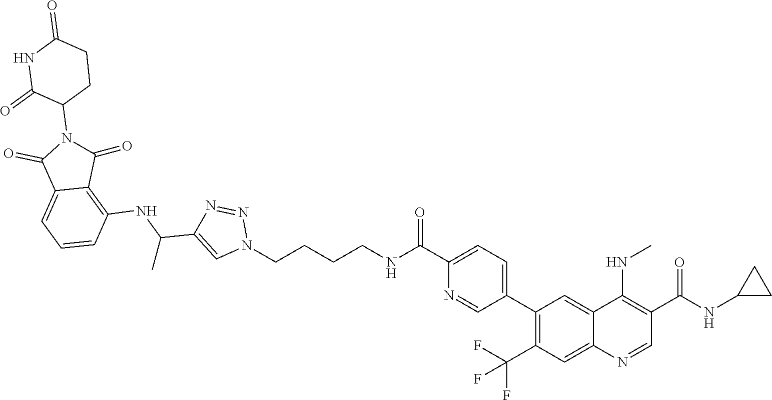 [SMILES: CNc1c(C(=O)NC2CC2)cnc2cc(C(F)(F)F)c(-c3ccc(C(=O)NCCCCn4cc(C(C)Nc5cccc6c5C(=O)N(C5CCC(=O)NC5=O)C6=O)nn4)nc3)cc12] |
| | N-cyclopropyl-6-(6-((4-(4-(1-((2-(2,6-dioxopiperidin-3-yl)-1,3- |
| | dioxoisoindolin-4-yl)amino)ethyl)-1H-1,2,3-triazol-1- |
| | yl)butyl)carbamoyl)pyridin-3-yl)-4-(methylamino)-7- |
| | (trifluoromethyl)quinoline-3-carboxamide |
| 432 | 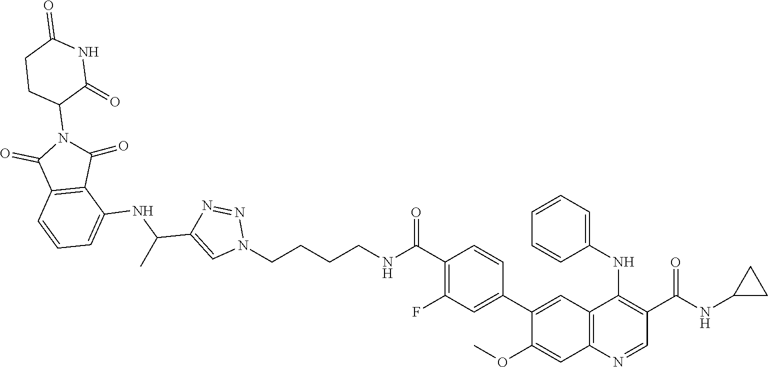 [SMILES: COc1cc2ncc(C(=O)NC3CC3)c(Nc3ccccc3)c2cc1-c1ccc(C(=O)NCCCCn2cc(C(C)Nc3cccc4c3C(=O)N(C3CCC(=O)NC3=O)C4=O)nn2)c(F)c1] |
| | N-cyclopropyl-6-(4-((4-(4-(1-((2-(2,6-dioxopiperidin-3-yl)-1,3- |
| | dioxoisoindolin-4-yl)amino)ethyl)-1H-1,2,3-triazol-1- |
| | yl)butyl)carbamoyl)-3-fluorophenyl)-7-methoxy-4- |
| | (phenylamino)quinoline-3-carboxamide |
| 433 | 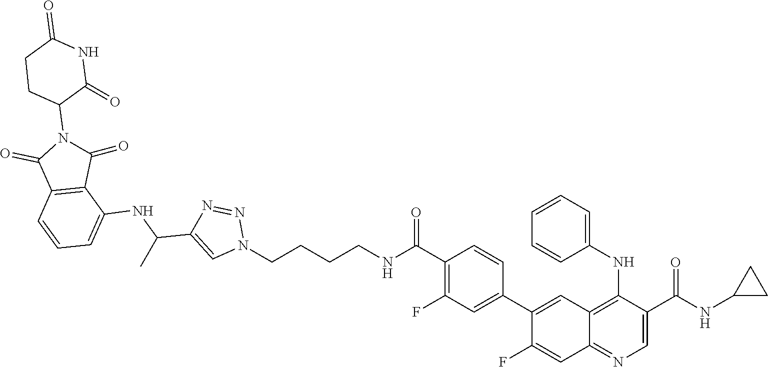 [SMILES: CC(Nc1cccc2c1C(=O)N(C1CCC(=O)NC1=O)C2=O)c1cn(CCCCNC(=O)c2ccc(-c3cc4c(Nc5ccccc5)c(C(=O)NC5CC5)cnc4cc3F)cc2F)nn1] |
| | N-cyclopropyl-6-(4-((4-(4-(1-((2-(2,6-dioxopiperidin-3-yl)-1,3- |
| | dioxoisoindolin-4-yl)amino)ethyl)-1H-1,2,3-triazol-1- |
| | yl)butyl)carbamoyl)-3-fluorophenyl)-7-fluoro-4- |
| | (phenylamino)quinoline-3-carboxamide |
| 434 | 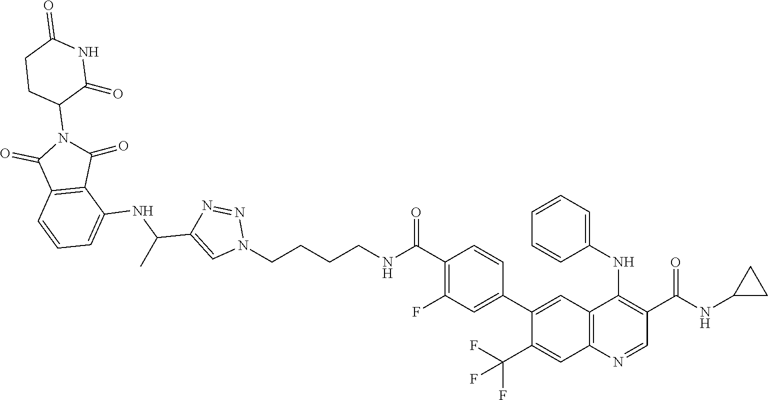 [SMILES: CC(Nc1cccc2c1C(=O)N(C1CCC(=O)NC1=O)C2=O)c1cn(CCCCNC(=O)c2ccc(-c3cc4c(Nc5ccccc5)c(C(=O)NC5CC5)cnc4cc3C(F)(F)F)cc2F)nn1] |
| | N-cyclopropyl-6-(4-((4-(4-(1-((2-(2,6-dioxopiperidin-3-yl)-1,3- |
| | dioxoisoindolin-4-yl)amino)ethyl)-1H-1,2,3-triazol-1- |
| | yl)butyl)carbamoyl)-3-fluorophenyl)-4-(phenylamino)-7- |
| | (trifluoromethyl)quinoline-3-carboxamide |
| 435 | 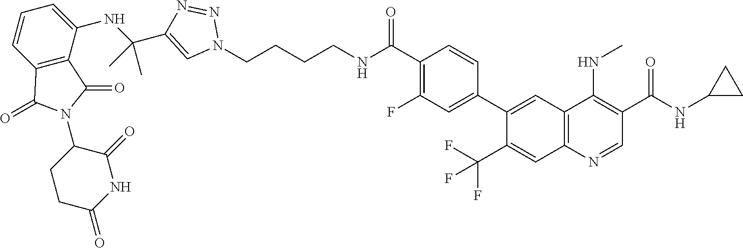 [SMILES: CNc1c(C(=O)NC2CC2)cnc2cc(C(F)(F)F)c(-c3ccc(C(=O)NCCCCn4cc(C(C)(C)Nc5cccc6c5C(=O)N(C5CCC(=O)NC5=O)C6=O)nn4)c(F)c3)cc12] |
| | N-cyclopropyl-6-(4-((4-(4-(2-((2-(2,6-dioxopiperidin-3-yl)-1,3- |
| | dioxoisoindolin-4-yl)amino)propan-2-yl)-1H-1,2,3-triazol-1- |
| | yl)butyl)carbamoyl)-3-fluorophenyl)-4-(methylamino)-7- |
| | (trifluoromethyl)quinoline-3-carboxamide |
| 436 | 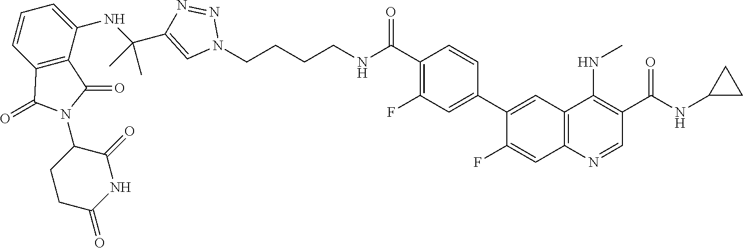 [SMILES: CNc1c(C(=O)NC2CC2)cnc2cc(F)c(-c3ccc(C(=O)NCCCCn4cc(C(C)(C)Nc5cccc6c5C(=O)N(C5CCC(=O)NC5=O)C6=O)nn4)c(F)c3)cc12] |
| | N-cyclopropyl-6-(4-((4-(4-(2-((2-(2,6-dioxopiperidin-3-yl)-1,3- |
| | dioxoisoindolin-4-yl)amino)propan-2-yl)-1H-1,2,3-triazol-1- |
| | yl)butyl)carbamoyl)-3-fluorophenyl)-7-fluoro-4- |
| | (methylamino)quinoline-3-carboxamide |
| 437 | 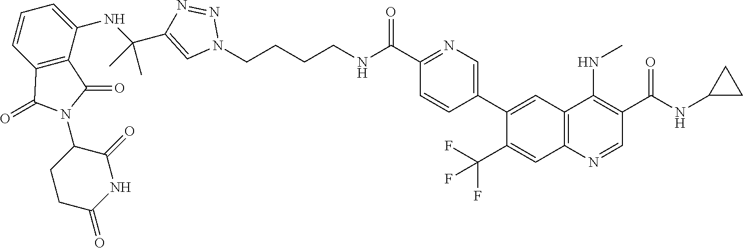 [SMILES: CNc1c(C(=O)NC2CC2)cnc2cc(C(F)(F)F)c(-c3ccc(C(=O)NCCCCn4cc(C(C)(C)Nc5cccc6c5C(=O)N(C5CCC(=O)NC5=O)C6=O)nn4)nc3)cc12] |
| | N-cyclopropyl-6-(6-((4-(4-(2-((2-(2,6-dioxopiperidin-3-yl)-1,3- |
| | dioxoisoindolin-4-yl)amino)propan-2-yl)-1H-1,2,3-triazol-1- |
| | yl)butyl)carbamoyl)pyridin-3-yl)-4-(methylamino)-7- |
| | (trifluoromethyl)quinoline-3-carboxamide |
| 439 | 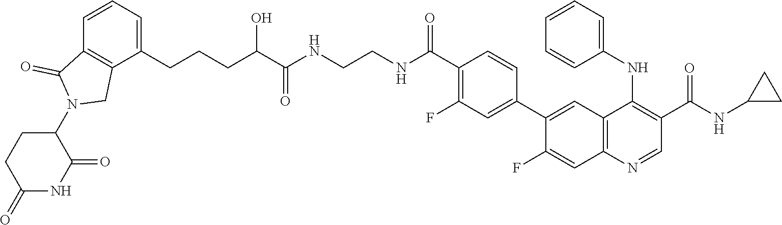 [SMILES: O=C1CCC(N2Cc3c(CCCC(O)C(=O)NCCNC(=O)c4ccc(-c5cc6c(Nc7ccccc7)c(C(=O)NC7CC7)cnc6cc5F)cc4F)cccc3C2=O)C(=O)N1] |
| | N-cyclopropyl-6-(4-((2-(5-(2-(2,6-dioxopiperidin-3-yl)-1-oxoisoindolin- |
| | 4-yl)-2-hydroxypentanamido)ethyl)carbamoyl)-3-fluorophenyl)-7-fluoro- |
| | 4-(phenylamino)quinoline-3-carboxamide |
| 440 | 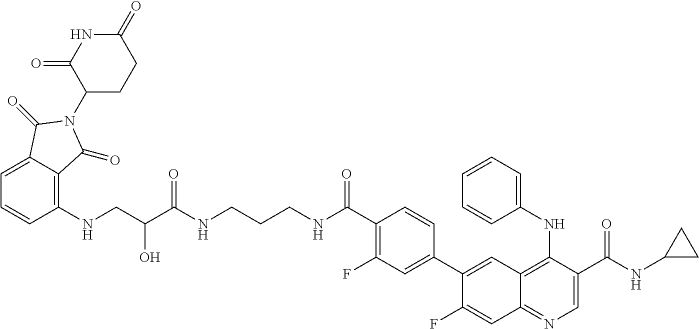 [SMILES: O=C1CCC(N2C(=O)c3cccc(NCC(O)C(=O)NCCCNC(=O)c4ccc(-c5cc6c(Nc7ccccc7)c(C(=O)NC7CC7)cnc6cc5F)cc4F)c3C2=O)C(=O)N1] |
| | N-cyclopropyl-6-(4-((3-(3-((2-(2,6-dioxopiperidin-3-yl)-1,3- |
| | dioxoisoindolin-4-yl)amino)-2-hydroxypropanamido)propyl)carbamoyl)- |
| | 3-fluorophenyl)-7-fluoro-4-(phenylamino)quinoline-3-carboxamide |
| 441 | 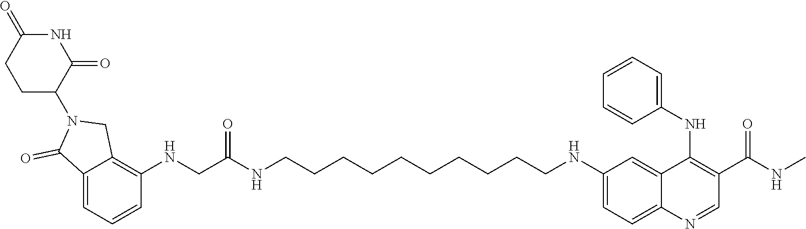 [SMILES: CNC(=O)c1cnc2ccc(NCCCCCCCCCCNC(=O)CNc3cccc4c3CN(C3CCC(=O)NC3=O)C4=O)cc2c1Nc1ccccc1] |
| | 6-((10-(2-((2-(2,6-dioxopiperidin-3-yl)-1-oxoisoindolin-4- |
| | yl)amino)acetamido)decyl)amino)-N-methyl-4-(phenylamino)quinoline- |
| | 3-carboxamide |
| 442 | 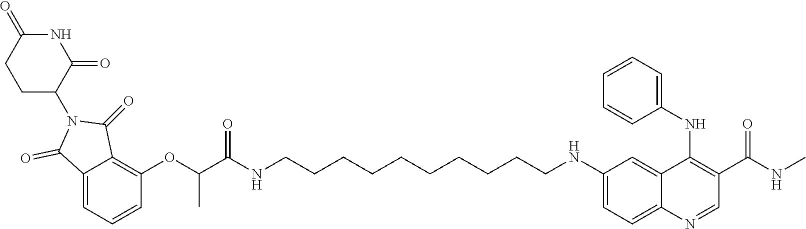 [SMILES: CNC(=O)c1cnc2ccc(NCCCCCCCCCCNC(=O)C(C)Oc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)oxy)propanamido)decyl)amino)-N-methyl-4-(phenylamino)quinoline- |
| | 3-carboxamide |
| 443 | 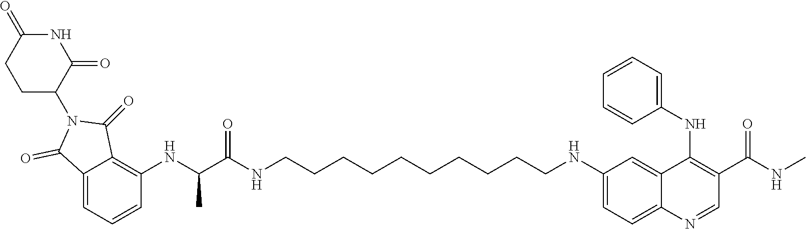 [SMILES: CNC(=O)c1cnc2ccc(NCCCCCCCCCCNC(=O)[C@@H](C)Nc3cccc4c3C(=O)N(C3CCC(=O)NC3=O)C4=O)cc2c1Nc1ccccc1] |
| | 6-((10-((2R)-2-((2-(2,6-dioxopiperidin-3-yl)-1,3-dioxoisoindolin-4- |
| | yl)amino)propanamido)decyl)amino)-N-methyl-4- |
| | (phenylamino)quinoline-3-carboxamide |
| 444 | 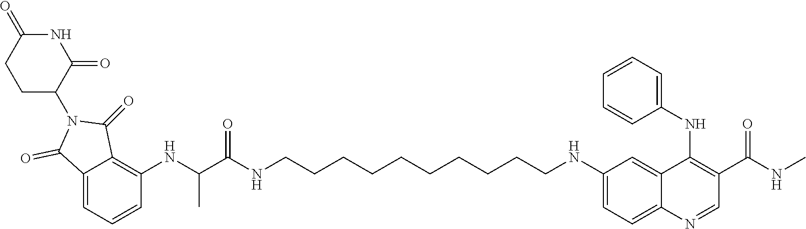 [SMILES: CNC(=O)c1cnc2ccc(NCCCCCCCCCCNC(=O)C(C)Nc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)amino)propanamido)decyl)amino)-N-methyl-4- |
| | (phenylamino)quinoline-3-carboxamide |
| 445 | 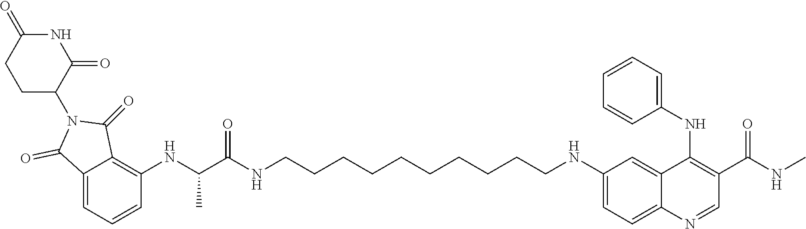 [SMILES: CNC(=O)c1cnc2ccc(NCCCCCCCCCCNC(=O)[C@H](C)Nc3cccc4c3C(=O)N(C3CCC(=O)NC3=O)C4=O)cc2c1Nc1ccccc1] |
| | 6-((10-((2S)-2-((2-(2,6-dioxopiperidin-3-yl)-1,3-dioxoisoindolin-4- |
| | yl)amino)propanamido)decyl)amino)-N-methyl-4- |
| | (phenylamino)quinoline-3-carboxamide |
| 446 | 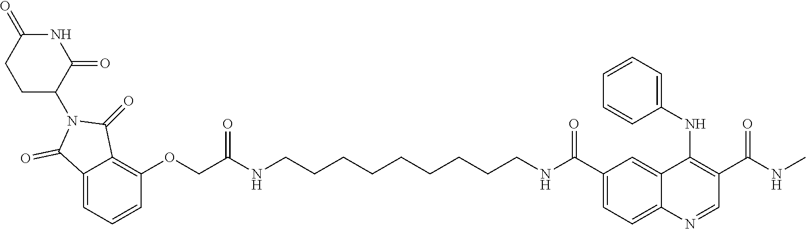 [SMILES: CNC(=O)c1cnc2ccc(C(=O)NCCCCCCCCCNC(=O)COc3cccc4c3C(=O)N(C3CCC(=O)NC3=O)C4=O)cc2c1Nc1ccccc1] |
| | N6-(9-(2-((2-(2,6-dioxopiperidin-3-yl)-1,3-dioxoisoindolin-4- |
| | yl)oxy)acetamido)nonyl)-N3-methyl-4-(phenylamino)quinoline-3,6- |
| | dicarboxamide |
| 447 | 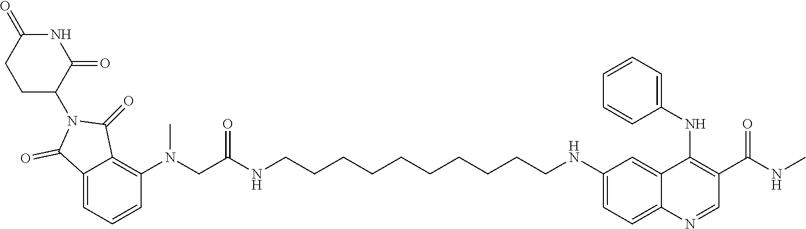 [SMILES: CNC(=O)c1cnc2ccc(NCCCCCCCCCCNC(=O)CN(C)c3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)(methyl)amino)acetamido)decyl)amino)-N-methyl-4- |
| | (phenylamino)quinoline-3-carboxamide |
| 448 | 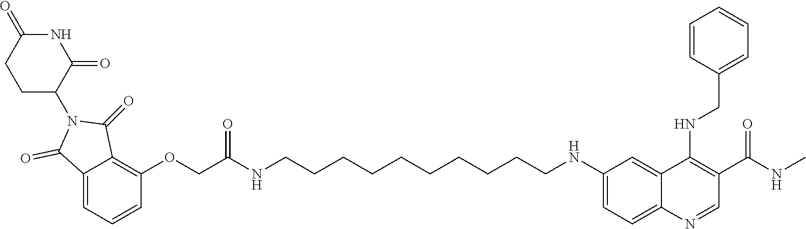 [SMILES: CNC(=O)c1cnc2ccc(NCCCCCCCCCCNC(=O)COc3cccc4c3C(=O)N(C3CCC(=O)NC3=O)C4=O)cc2c1NCc1ccccc1] |
| | 4-(benzylamino)-6-((10-(2-((2-(2,6-dioxopiperidin-3-yl)-1,3- |
| | dioxoisoindolin-4-yl)oxy)acetamido)decyl)amino)-N-methylquinoline-3- |
| | carboxamide |
| 449 | 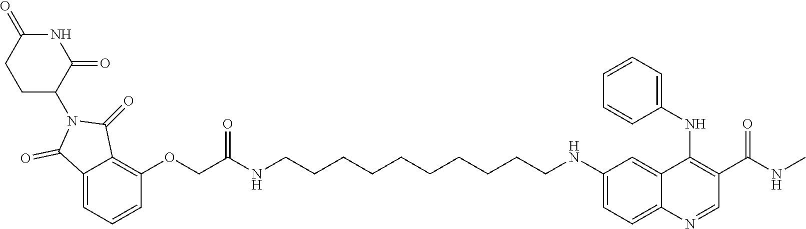 [SMILES: CNC(=O)c1cnc2ccc(NCCCCCCCCCCNC(=O)COc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)oxy)acetamido)decyl)amino)-N-methyl-4-(phenylamino)quinoline-3- |
| | carboxamide |
| 450 | 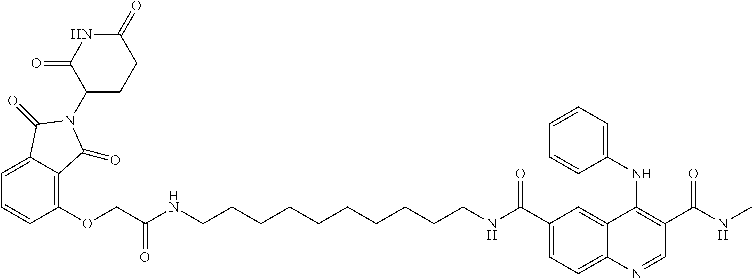 [SMILES: CNC(=O)c1cnc2ccc(C(=O)NCCCCCCCCCCNC(=O)COc3cccc4c3C(=O)N(C3CCC(=O)NC3=O)C4=O)cc2c1Nc1ccccc1] |
| | N6-(10-(2-((2-(2,6-dioxopiperidin-3-yl)-1,3-dioxoisoindolin-4- |
| | yl)oxy)acetamido)decyl)-N3-methyl-4-(phenylamino)quinoline-3,6- |
| | dicarboxamide |
| 451 | 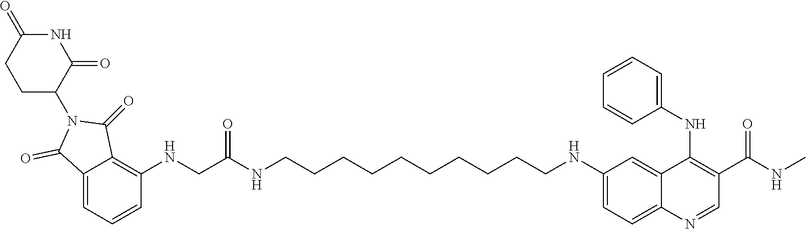 [SMILES: CNC(=O)c1cnc2ccc(NCCCCCCCCCCNC(=O)CNc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)amino)acetamido)decyl)amino)-N-methyl-4-(phenylamino)quinoline- |
| | 3-carboxamide |
| 452 | 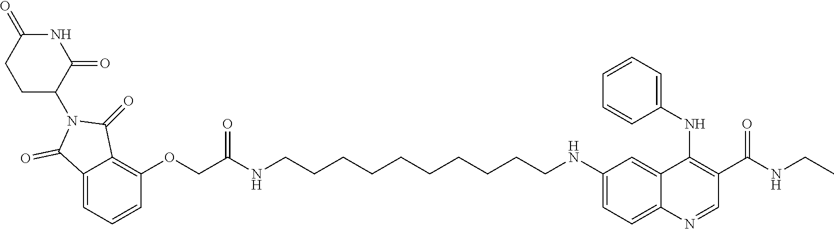 [SMILES: CCNC(=O)c1cnc2ccc(NCCCCCCCCCCNC(=O)COc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)oxy)acetamido)decyl)amino)-N-ethyl-4-(phenylamino)quinoline-3- |
| | carboxamide |
| 453 | 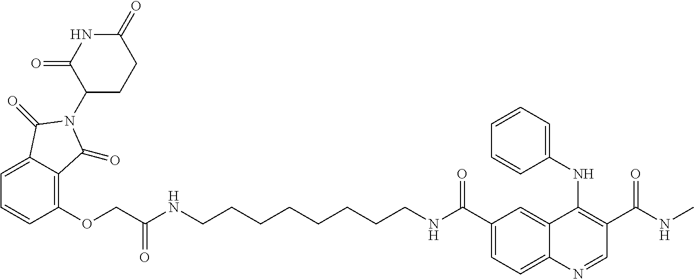 [SMILES: CNC(=O)c1cnc2ccc(C(=O)NCCCCCCCCNC(=O)COc3cccc4c3C(=O)N(C3CCC(=O)NC3=O)C4=O)cc2c1Nc1ccccc1] |
| | N6-(8-(2-((2-(2,6-dioxopiperidin-3-yl)-1,3-dioxoisoindolin-4- |
| | yl)oxy)acetamido)octyl)-N3-methyl-4-(phenylamino)quinoline-3,6- |
| | dicarboxamide |
| 454 | 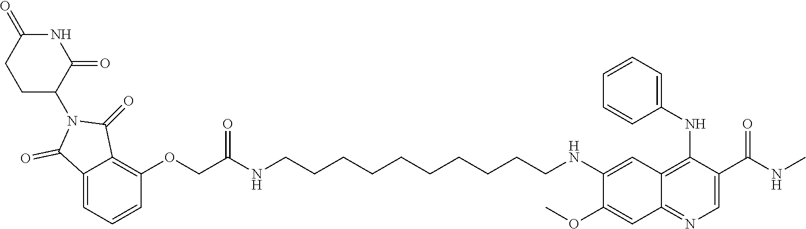 [SMILES: CNC(=O)c1cnc2cc(OC)c(NCCCCCCCCCCNC(=O)COc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)oxy)acetamido)decyl)amino)-7-methoxy-N-methyl-4- |
| | (phenylamino)quinoline-3-carboxamide |
| 455 | 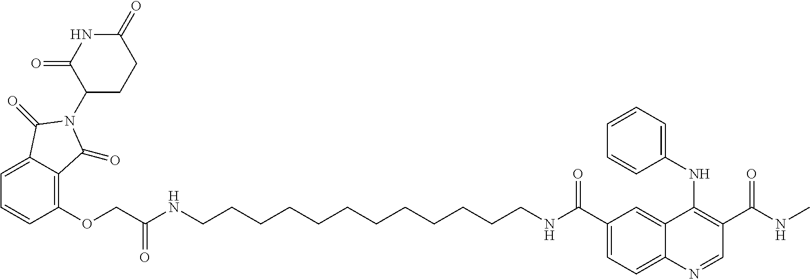 [SMILES: CNC(=O)c1cnc2ccc(C(=O)NCCCCCCCCCCCCNC(=O)COc3cccc4c3C(=O)N(C3CCC(=O)NC3=O)C4=O)cc2c1Nc1ccccc1] |
| | N6-(12-(2-((2-(2,6-dioxopiperidin-3-yl)-1,3-dioxoisoindolin-4- |
| | yl)oxy)acetamido)dodecyl)-N3-methyl-4-(phenylamino)quinoline-3,6- |
| | dicarboxamide |
| 456 | 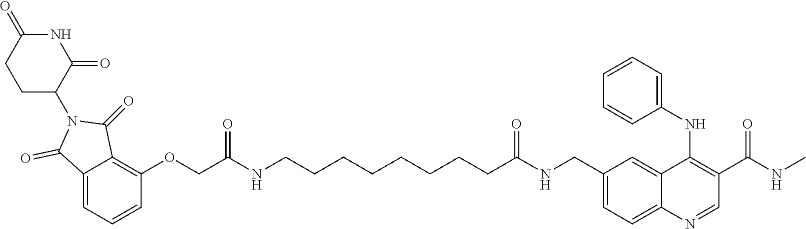 [SMILES: CNC(=O)c1cnc2ccc(CNC(=O)CCCCCCCCNC(=O)COc3cccc4c3C(=O)N(C3CCC(=O)NC3=O)C4=O)cc2c1Nc1ccccc1] |
| | 6-((9-(2-((2-(2,6-dioxopiperidin-3-yl)-1,3-dioxoisoindolin-4- |
| | yl)oxy)acetamido)nonanamido)methyl)-N-methyl-4- |
| | (phenylamino)quinoline-3-carboxamide |
| 457 | 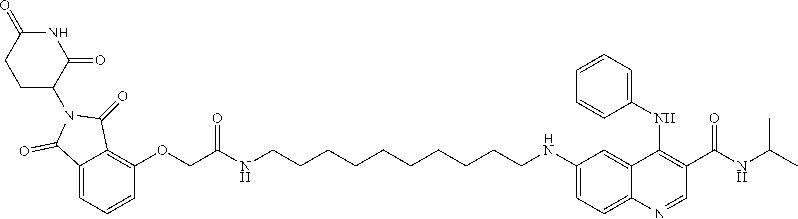 [SMILES: CC(C)NC(=O)c1cnc2ccc(NCCCCCCCCCCNC(=O)COc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)oxy)acetamido)decyl)amino)-N-isopropyl-4-(phenylamino)quinoline- |
| | 3-carboxamide |
| 458 | 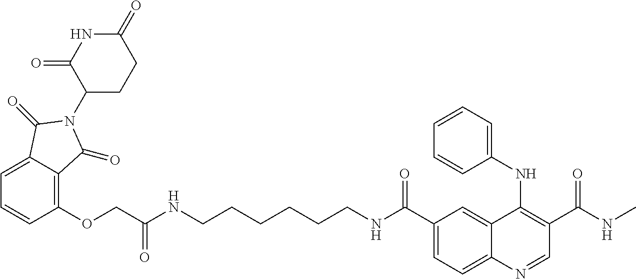 [SMILES: CNC(=O)c1cnc2ccc(C(=O)NCCCCCCNC(=O)COc3cccc4c3C(=O)N(C3CCC(=O)NC3=O)C4=O)cc2c1Nc1ccccc1] |
| | N6-(6-(2-((2-(2,6-dioxopiperidin-3-yl)-1,3-dioxoisoindolin-4- |
| | yl)oxy)acetamido)hexyl)-N3-methyl-4-(phenylamino)quinoline-3,6- |
| | dicarboxamide |
| 459 | 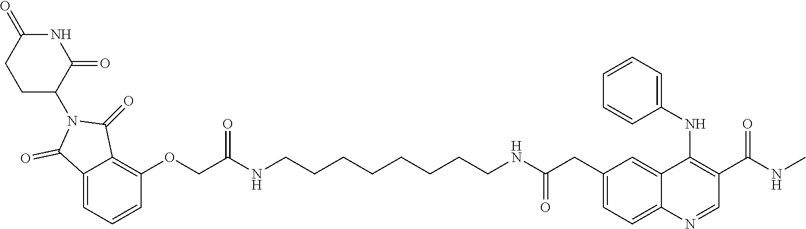 [SMILES: CNC(=O)c1cnc2ccc(CC(=O)NCCCCCCCCNC(=O)COc3cccc4c3C(=O)N(C3CCC(=O)NC3=O)C4=O)cc2c1Nc1ccccc1] |
| | 6-(2-((8-(2-((2-(2,6-dioxopiperidin-3-yl)-1,3-dioxoisoindolin-4- |
| | yl)oxy)acetamido)octyl)amino)-2-oxoethyl)-N-methyl-4- |
| | (phenylamino)quinoline-3-carboxamide |
| 460 | 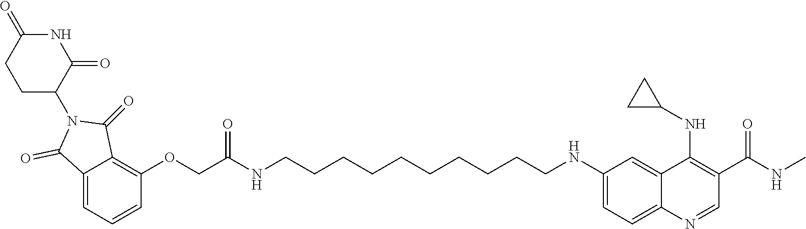 [SMILES: CNC(=O)c1cnc2ccc(NCCCCCCCCCCNC(=O)COc3cccc4c3C(=O)N(C3CCC(=O)NC3=O)C4=O)cc2c1NC1CC1] |
| | 4-(cyclopropylamino)-6-((10-(2-((2-(2,6-dioxopiperidin-3-yl)-1,3- |
| | dioxoisoindolin-4-yl)oxy)acetamido)decyl)amino)-N-methylquinoline-3- |
| | carboxamide |
| 464 | 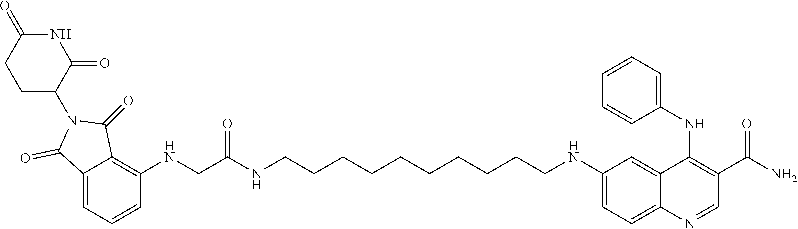 [SMILES: NC(=O)c1cnc2ccc(NCCCCCCCCCCNC(=O)CNc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)amino)acetamido)decyl)amino)-4-(phenylamino)quinoline-3- |
| | carboxamide |
| 465 | 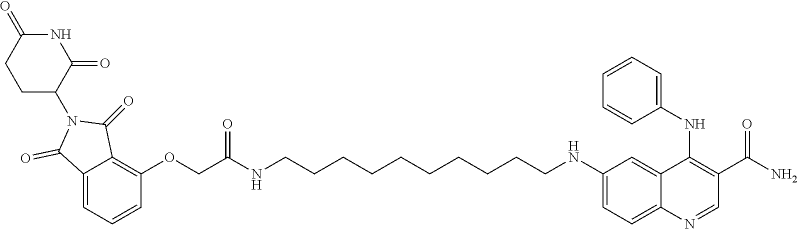 [SMILES: NC(=O)c1cnc2ccc(NCCCCCCCCCCNC(=O)COc3cccc4c3C(=O)N(C3CCC(=O)NC3=O)C4=O)cc2c1Nc1ccccc1] |
| | 6-((10-(2-((2-(2,6-dioxopiperidin-3-yl)-1,3-dioxoisoindolin-4- |
| | yl)oxy)acetamido)decyl)amino)-4-(phenylamino)quinoline-3- |
| | carboxamide |
| 466 | 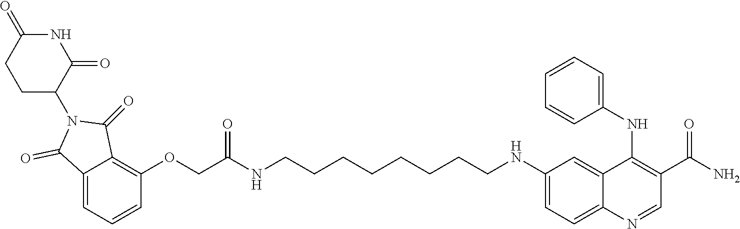 [SMILES: NC(=O)c1cnc2ccc(NCCCCCCCCNC(=O)COc3cccc4c3C(=O)N(C3CCC(=O)NC3=O)C4=O)cc2c1Nc1ccccc1] |
| | 6-((8-(2-((2-(2,6-dioxopiperidin-3-yl)-1,3-dioxoisoindolin-4- |
| | yl)oxy)acetamido)octyl)amino)-4-(phenylamino)quinoline-3- |
| | carboxamide |
| 467 | 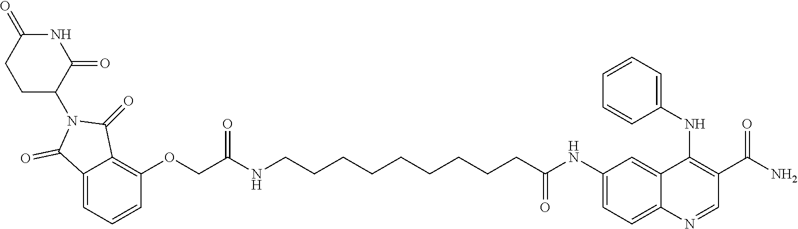 [SMILES: NC(=O)c1cnc2ccc(NC(=O)CCCCCCCCCNC(=O)COc3cccc4c3C(=O)N(C3CCC(=O)NC3=O)C4=O)cc2c1Nc1ccccc1] |
| | 6-(10-(2-((2-(2,6-dioxopiperidin-3-yl)-1,3-dioxoisoindolin-4- |
| | yl)oxy)acetamido)decanamido)-4-(phenylamino)quinoline-3- |
| | carboxamide |
| 471 | 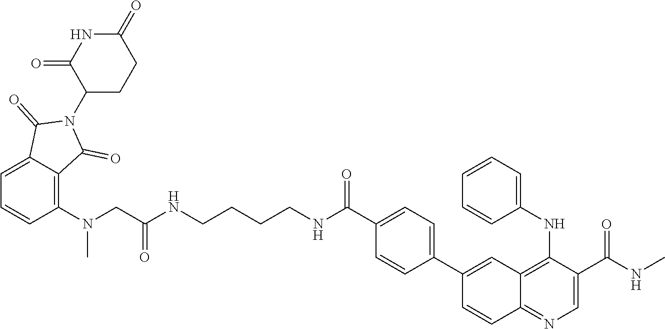 [SMILES: CNC(=O)c1cnc2ccc(-c3ccc(C(=O)NCCCCNC(=O)CN(C)c4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)(methyl)amino)acetamido)butyl)carbamoyl)phenyl)-N-methyl-4- |
| | (phenylamino)quinoline-3-carboxamide |
| 472 | 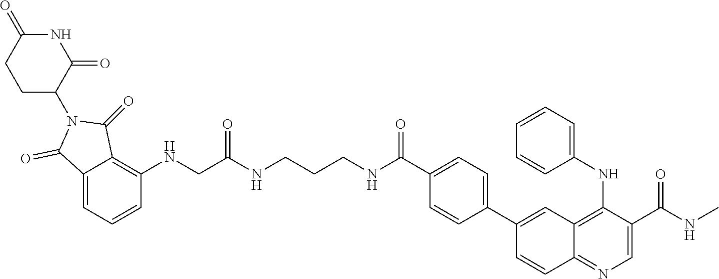 [SMILES: CNC(=O)c1cnc2ccc(-c3ccc(C(=O)NCCCNC(=O)CNc4cccc5c4C(=O)N(C4CCC(=O)NC4=O)C5=O)cc3)cc2c1Nc1ccccc1] |
| | 6-(4-((3-(2-((2-(2,6-dioxopiperidin-3-yl)-1,3-dioxoisoindolin-4- |
| | yl)amino)acetamido)propyl)carbamoyl)phenyl)-N-methyl-4- |
| | (phenylamino)quinoline-3-carboxamide |
| 473 | 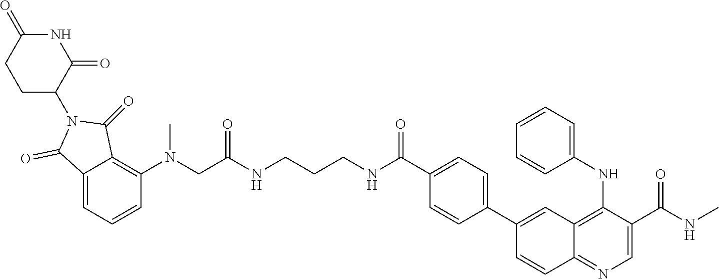 [SMILES: CNC(=O)c1cnc2ccc(-c3ccc(C(=O)NCCCNC(=O)CN(C)c4cccc5c4C(=O)N(C4CCC(=O)NC4=O)C5=O)cc3)cc2c1Nc1ccccc1] |
| | 6-(4-((3-(2-((2-(2,6-dioxopiperidin-3-yl)-1,3-dioxoisoindolin-4- |
| | yl)(methyl)amino)acetamido)propyl)carbamoyl)phenyl)-N-methyl-4- |
| | (phenylamino)quinoline-3-carboxamide |
| 474 | 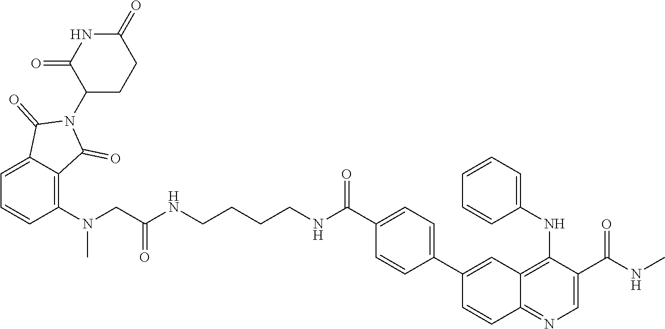 [SMILES: CNC(=O)c1cnc2ccc(-c3ccc(C(=O)NCCCCNC(=O)CN(C)c4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)(methyl)amino)acetamido)butyl)carbamoyl)phenyl)-N-methyl-4- |
| | (phenylamino)quinoline-3-carboxamide |
| 475 | 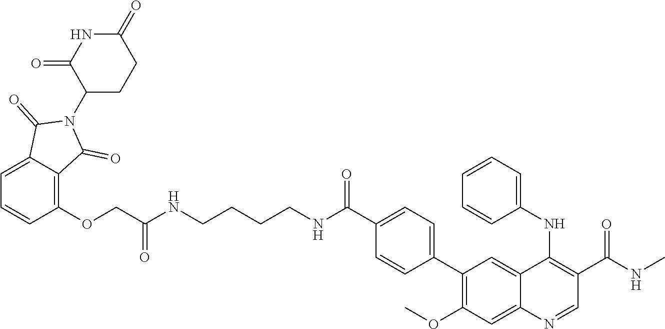 [SMILES: CNC(=O)c1cnc2cc(OC)c(-c3ccc(C(=O)NCCCCNC(=O)COc4cccc5c4C(=O)N(C4CCC(=O)NC4=O)C5=O)cc3)cc2c1Nc1ccccc1] |
| | 6-(4-((4-(2-((2-(2,6-dioxopiperidin-3-yl)-1,3-dioxoisoindolin-4- |
| | yl)oxy)acetamido)butyl)carbamoyl)phenyl)-7-methoxy-N-methyl-4- |
| | (phenylamino)quinoline-3-carboxamide |

or a pharmaceutically acceptable salt thereof.

The BRD9 inhibitor may be, e.g., a compound selected from the group consisting of:

| Compound | |
|---|---|
| Number | Compound Structure |
| A1 | 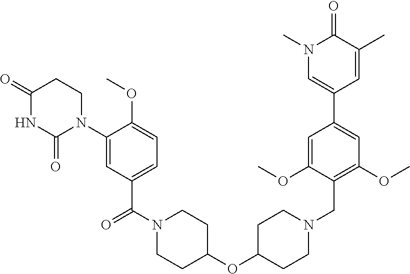 [SMILES: COc1ccc(C(=O)N2CCC(OC3CCN(Cc4c(OC)cc(-c5cc(C)c(=O)n(C)c5)cc4OC)CC3)CC2)cc1N1CCC(=O)NC1=O] |
| A2 | 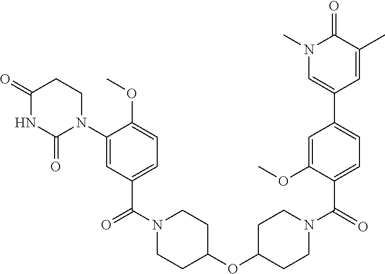 [SMILES: COc1cc(-c2cc(C)c(=O)n(C)c2)ccc1C(=O)N1CCC(OC2CCN(C(=O)c3ccc(OC)c(N4CCC(=O)NC4=O)c3)CC2)CC1] |
| A3 | 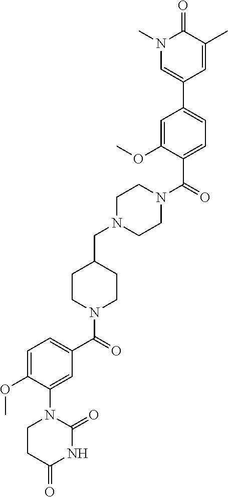 [SMILES: COc1cc(-c2cc(C)c(=O)n(C)c2)ccc1C(=O)N1CCN(CC2CCN(C(=O)c3ccc(OC)c(N4CCC(=O)NC4=O)c3)CC2)CC1] |
| A4 | 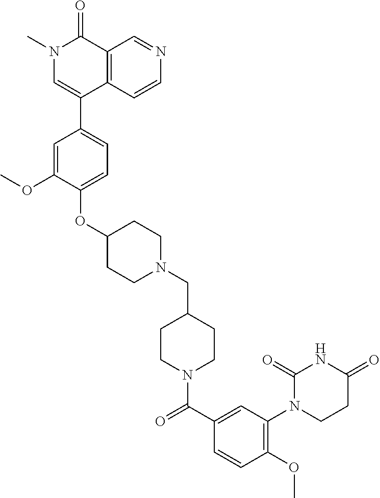 [SMILES: COc1cc(-c2cn(C)c(=O)c3cnccc23)ccc1OC1CCN(CC2CCN(C(=O)c3ccc(OC)c(N4CCC(=O)NC4=O)c3)CC2)CC1] |
| A5 | 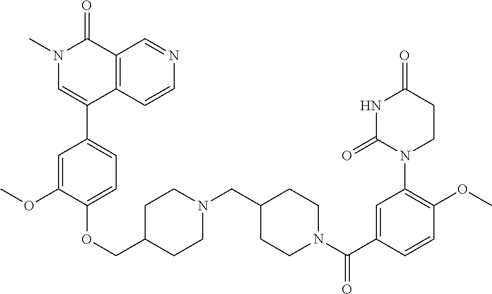 |
| A6 | 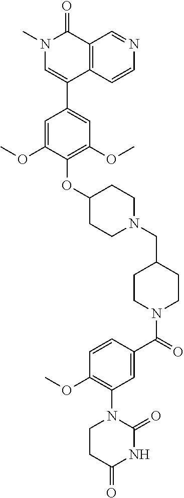 [SMILES: COc1ccc(C(=O)N2CCC(CN3CCC(Oc4c(OC)cc(-c5cn(C)c(=O)c6cnccc56)cc4OC)CC3)CC2)cc1N1CCC(=O)NC1=O] |
| A7 | 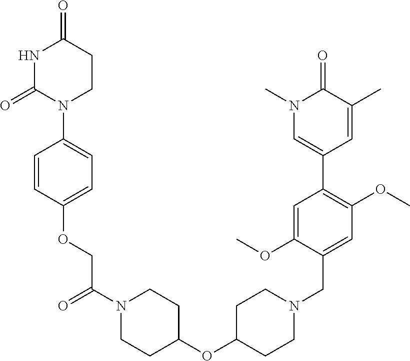 [SMILES: COc1cc(-c2cc(C)c(=O)n(C)c2)c(OC)cc1CN1CCC(OC2CCN(C(=O)COc3ccc(N4CCC(=O)NC4=O)cc3)CC2)CC1] |
| A8 | 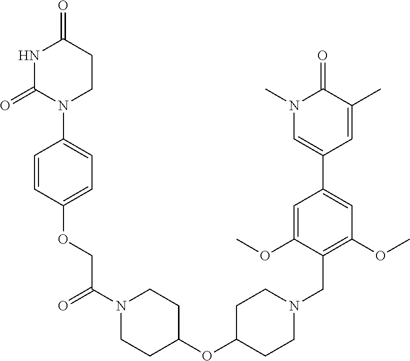 [SMILES: COc1cc(-c2cc(C)c(=O)n(C)c2)cc(OC)c1CN1CCC(OC2CCN(C(=O)COc3ccc(N4CCC(=O)NC4=O)cc3)CC2)CC1] |
| A9 | 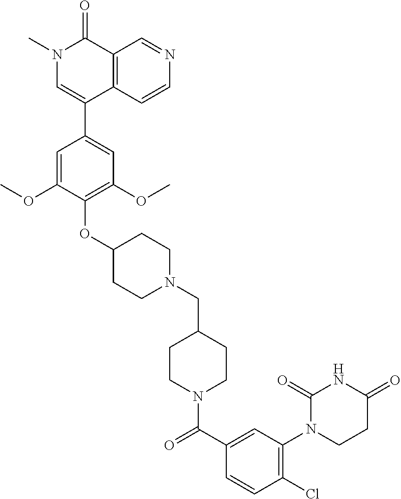 [SMILES: COc1cc(-c2cn(C)c(=O)c3cnccc23)cc(OC)c1OC1CCN(CC2CCN(C(=O)c3ccc(Cl)c(N4CCC(=O)NC4=O)c3)CC2)CC1] |
| A10 | 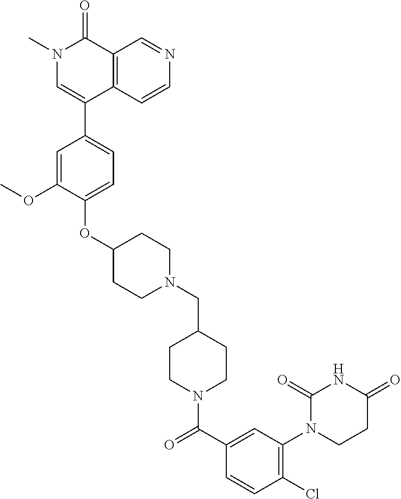 [SMILES: COc1cc(-c2cn(C)c(=O)c3cnccc23)ccc1OC1CCN(CC2CCN(C(=O)c3ccc(Cl)c(N4CCC(=O)NC4=O)c3)CC2)CC1] |
| A11 | 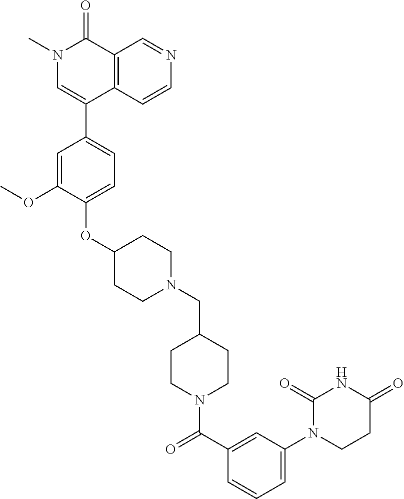 [SMILES: COc1cc(-c2cn(C)c(=O)c3cnccc23)ccc1OC1CCN(CC2CCN(C(=O)c3cccc(N4CCC(=O)NC4=O)c3)CC2)CC1] |
| A12 | 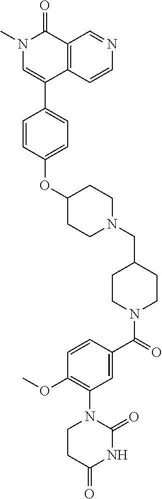 [SMILES: COc1ccc(C(=O)N2CCC(CN3CCC(Oc4ccc(-c5cn(C)c(=O)c6cnccc56)cc4)CC3)CC2)cc1N1CCC(=O)NC1=O] |
| A13 | 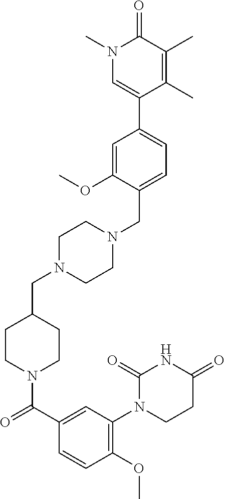 [SMILES: COc1cc(-c2cn(C)c(=O)c(C)c2C)ccc1CN1CCN(CC2CCN(C(=O)c3ccc(OC)c(N4CCC(=O)NC4=O)c3)CC2)CC1] |
| A14 | 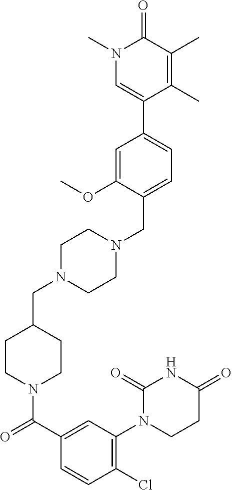 [SMILES: COc1cc(-c2cn(C)c(=O)c(C)c2C)ccc1CN1CCN(CC2CCN(C(=O)c3ccc(Cl)c(N4CCC(=O)NC4=O)c3)CC2)CC1] |
| A15 | 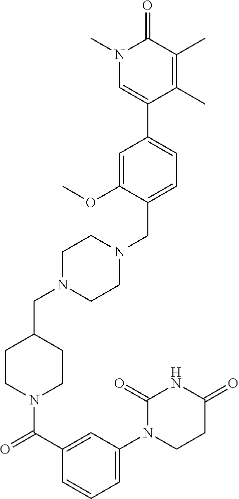 [SMILES: COc1cc(-c2cn(C)c(=O)c(C)c2C)ccc1CN1CCN(CC2CCN(C(=O)c3cccc(N4CCC(=O)NC4=O)c3)CC2)CC1] |
| A16 | 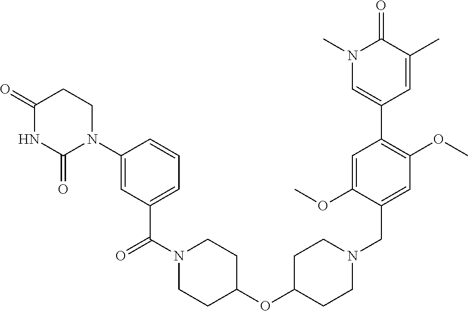 [SMILES: COc1cc(-c2cc(C)c(=O)n(C)c2)c(OC)cc1CN1CCC(OC2CCN(C(=O)c3cccc(N4CCC(=O)NC4=O)c3)CC2)CC1] |
| A17 | 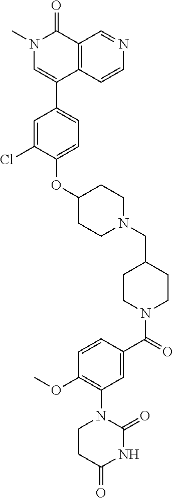 [SMILES: COc1ccc(C(=O)N2CCC(CN3CCC(Oc4ccc(-c5cn(C)c(=O)c6cnccc56)cc4Cl)CC3)CC2)cc1N1CCC(=O)NC1=O] |
| A18 | 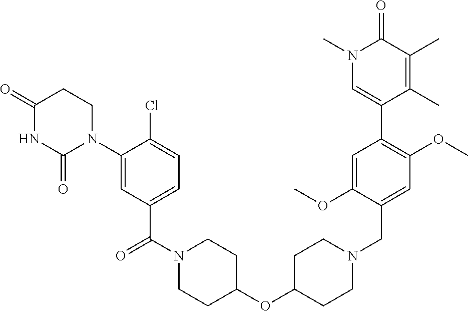 [SMILES: COc1cc(-c2cn(C)c(=O)c(C)c2C)c(OC)cc1CN1CCC(OC2CCN(C(=O)c3ccc(Cl)c(N4CCC(=O)NC4=O)c3)CC2)CC1] |
| A19 | 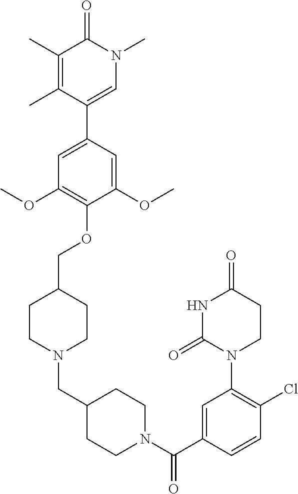 [SMILES: COc1cc(-c2cn(C)c(=O)c(C)c2C)cc(OC)c1OCC1CCN(CC2CCN(C(=O)c3ccc(Cl)c(N4CCC(=O)NC4=O)c3)CC2)CC1] |
| A20 | 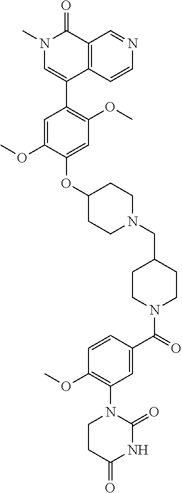 [SMILES: COc1cc(-c2cn(C)c(=O)c3cnccc23)c(OC)cc1OC1CCN(CC2CCN(C(=O)c3ccc(OC)c(N4CCC(=O)NC4=O)c3)CC2)CC1] |
| A21 | 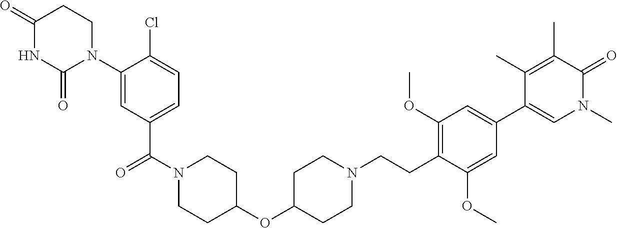 [SMILES: COc1cc(-c2cn(C)c(=O)c(C)c2C)cc(OC)c1CCN1CCC(OC2CCN(C(=O)c3ccc(Cl)c(N4CCC(=O)NC4=O)c3)CC2)CC1] |
| A22 | 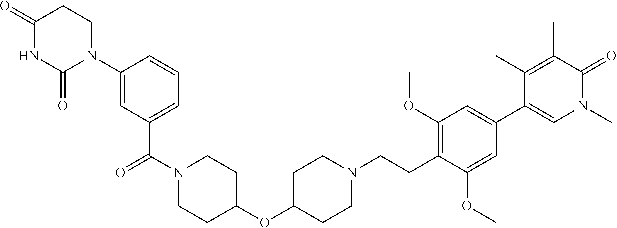 [SMILES: COc1cc(-c2cn(C)c(=O)c(C)c2C)cc(OC)c1CCN1CCC(OC2CCN(C(=O)c3cccc(N4CCC(=O)NC4=O)c3)CC2)CC1] |
| A23 | 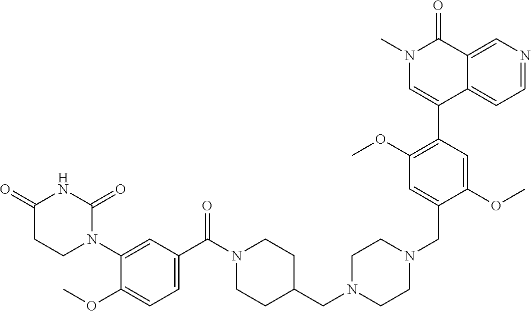 [SMILES: COc1cc(-c2cn(C)c(=O)c3cnccc23)c(OC)cc1CN1CCN(CC2CCN(C(=O)c3ccc(OC)c(N4CCC(=O)NC4=O)c3)CC2)CC1] |
| A24 | 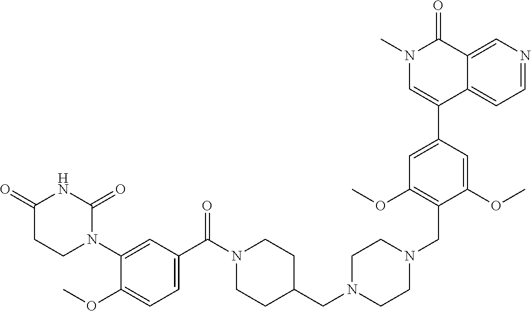 [SMILES: COc1ccc(C(=O)N2CCC(CN3CCN(Cc4c(OC)cc(-c5cn(C)c(=O)c6cnccc56)cc4OC)CC3)CC2)cc1N1CCC(=O)NC1=O] |
| A25 | 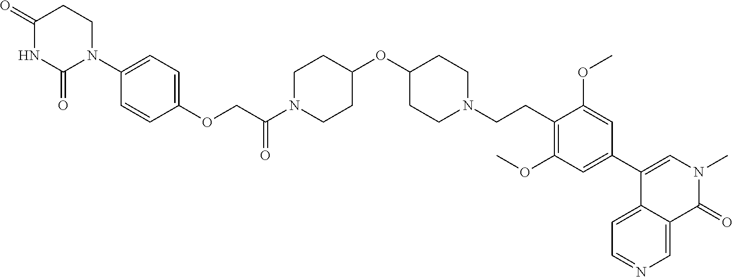 [SMILES: COc1cc(-c2cn(C)c(=O)c3cnccc23)cc(OC)c1CCN1CCC(OC2CCN(C(=O)COc3ccc(N4CCC(=O)NC4=O)cc3)CC2)CC1] |
| A26 | 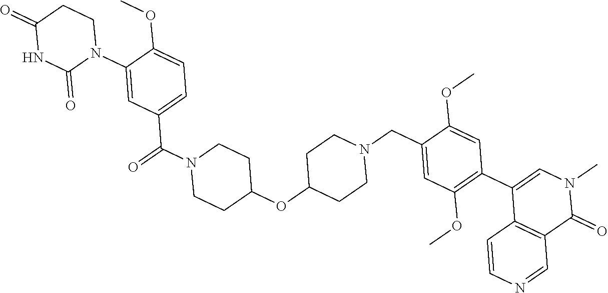 [SMILES: COc1cc(-c2cn(C)c(=O)c3cnccc23)c(OC)cc1CN1CCC(OC2CCN(C(=O)c3ccc(OC)c(N4CCC(=O)NC4=O)c3)CC2)CC1] |
| A27 | 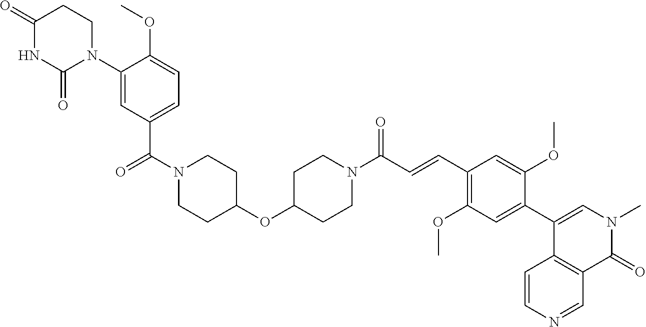 [SMILES: COc1cc(-c2cn(C)c(=O)c3cnccc23)c(OC)cc1/C=C/C(=O)N1CCC(OC2CCN(C(=O)c3ccc(OC)c(N4CCC(=O)NC4=O)c3)CC2)CC1] |
| A28 | 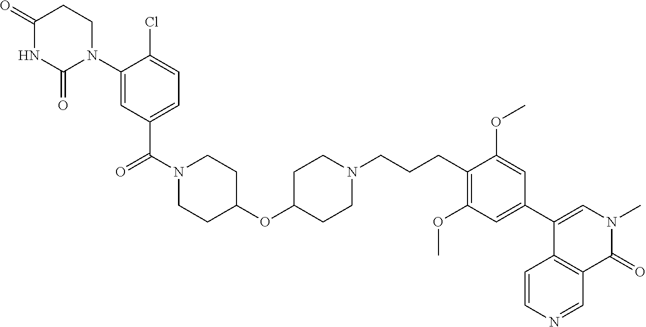 [SMILES: COc1cc(-c2cn(C)c(=O)c3cnccc23)cc(OC)c1CCCN1CCC(OC2CCN(C(=O)c3ccc(Cl)c(N4CCC(=O)NC4=O)c3)CC2)CC1] |
| A29 | 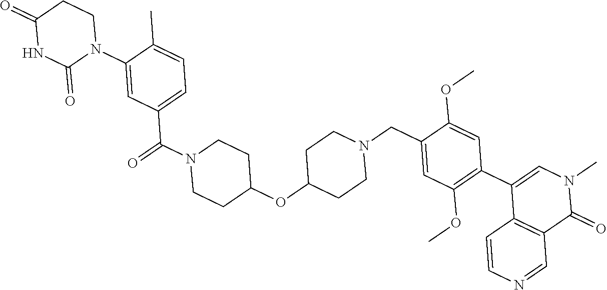 [SMILES: COc1cc(-c2cn(C)c(=O)c3cnccc23)c(OC)cc1CN1CCC(OC2CCN(C(=O)c3ccc(C)c(N4CCC(=O)NC4=O)c3)CC2)CC1] |
| A30 | 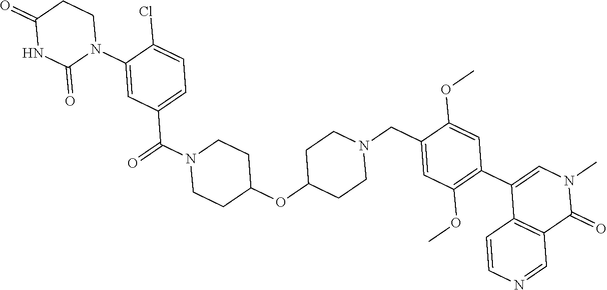 [SMILES: COc1cc(-c2cn(C)c(=O)c3cnccc23)c(OC)cc1CN1CCC(OC2CCN(C(=O)c3ccc(Cl)c(N4CCC(=O)NC4=O)c3)CC2)CC1] |
| A31 | 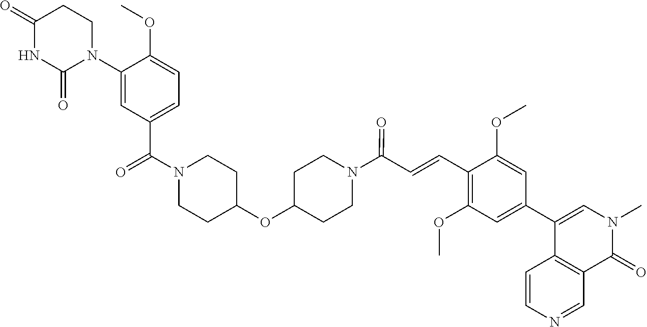 [SMILES: COc1ccc(C(=O)N2CCC(OC3CCN(C(=O)/C=C/c4c(OC)cc(-c5cn(C)c(=O)c6cnccc56)cc4OC)CC3)CC2)cc1N1CCC(=O)NC1=O] |
| A32 | 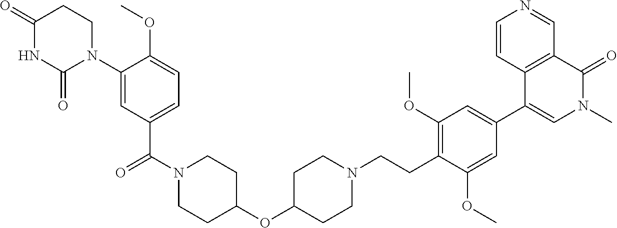 [SMILES: COc1ccc(C(=O)N2CCC(OC3CCN(CCc4c(OC)cc(-c5cn(C)c(=O)c6cnccc56)cc4OC)CC3)CC2)cc1N1CCC(=O)NC1=O] |
| A33 | 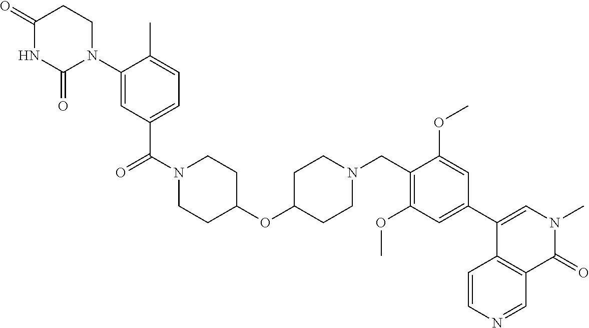 [SMILES: COc1cc(-c2cn(C)c(=O)c3cnccc23)cc(OC)c1CN1CCC(OC2CCN(C(=O)c3ccc(C)c(N4CCC(=O)NC4=O)c3)CC2)CC1] |
| A34 | 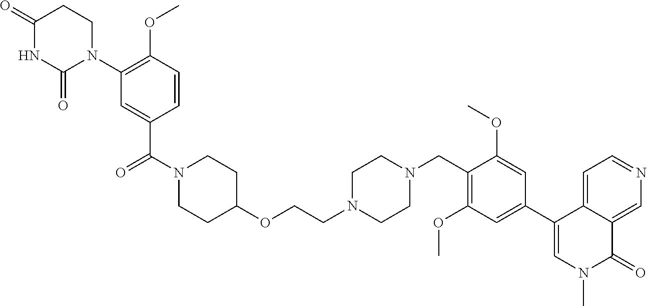 [SMILES: COc1ccc(C(=O)N2CCC(OCCN3CCN(Cc4c(OC)cc(-c5cn(C)c(=O)c6cnccc56)cc4OC)CC3)CC2)cc1N1CCC(=O)NC1=O] |
| A35 | 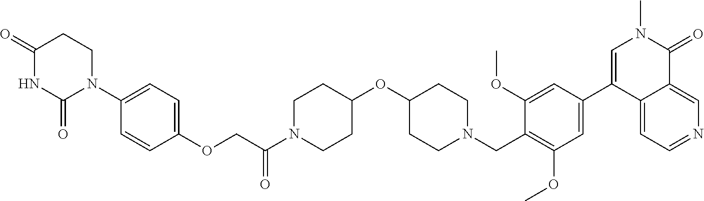 [SMILES: COc1cc(-c2cn(C)c(=O)c3cnccc23)cc(OC)c1CN1CCC(OC2CCN(C(=O)COc3ccc(N4CCC(=O)NC4=O)cc3)CC2)CC1] |
| A36 | 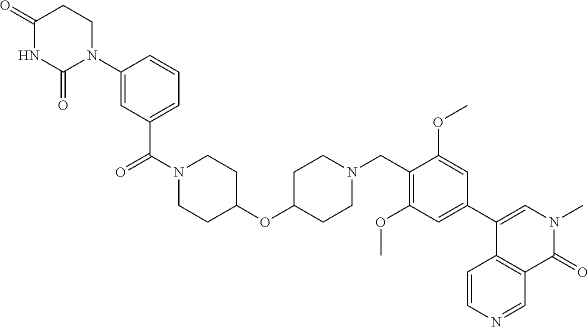 [SMILES: COc1cc(-c2cn(C)c(=O)c3cnccc23)cc(OC)c1CN1CCC(OC2CCN(C(=O)c3cccc(N4CCC(=O)NC4=O)c3)CC2)CC1] |
| A37 | 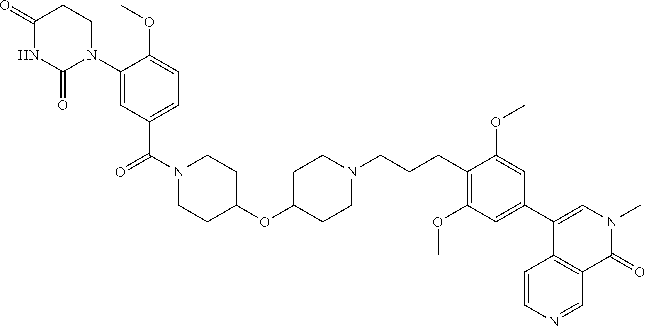 [SMILES: COc1ccc(C(=O)N2CCC(OC3CCN(CCCc4c(OC)cc(-c5cn(C)c(=O)c6cnccc56)cc4OC)CC3)CC2)cc1N1CCC(=O)NC1=O] |
| A38 | 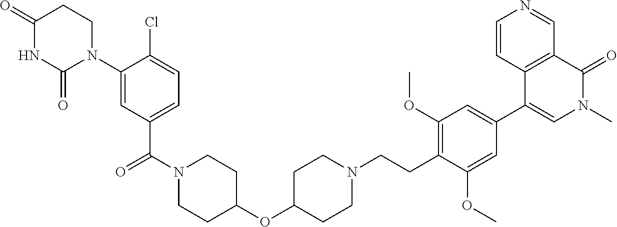 [SMILES: COc1cc(-c2cn(C)c(=O)c3cnccc23)cc(OC)c1CCN1CCC(OC2CCN(C(=O)c3ccc(Cl)c(N4CCC(=O)NC4=O)c3)CC2)CC1] |
| A39 | 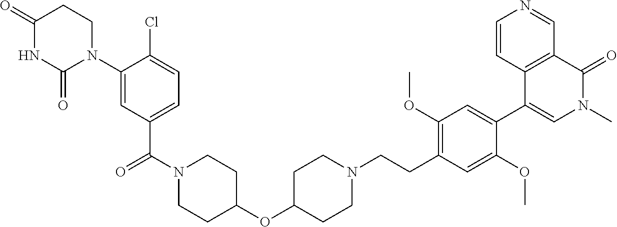 [SMILES: COc1cc(-c2cn(C)c(=O)c3cnccc23)c(OC)cc1CCN1CCC(OC2CCN(C(=O)c3ccc(Cl)c(N4CCC(=O)NC4=O)c3)CC2)CC1] |
| A40 | 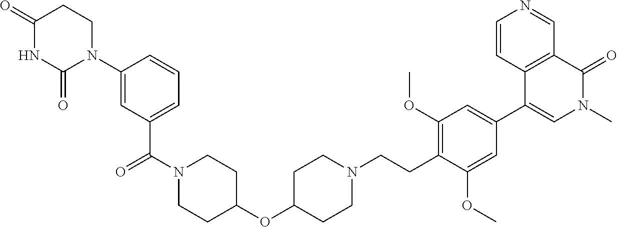 [SMILES: COc1cc(-c2cn(C)c(=O)c3cnccc23)cc(OC)c1CCN1CCC(OC2CCN(C(=O)c3cccc(N4CCC(=O)NC4=O)c3)CC2)CC1] |
| A41 | 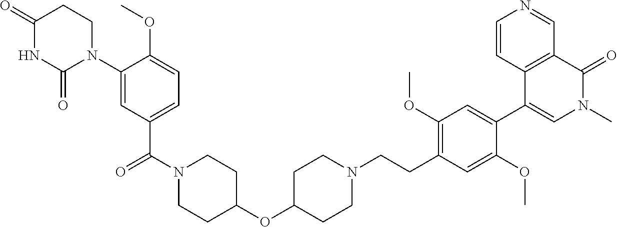 [SMILES: COc1cc(-c2cn(C)c(=O)c3cnccc23)c(OC)cc1CCN1CCC(OC2CCN(C(=O)c3ccc(OC)c(N4CCC(=O)NC4=O)c3)CC2)CC1] |
| A42 | 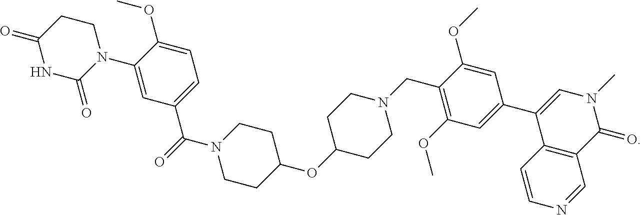 [SMILES: COc1ccc(C(=O)N2CCC(OC3CCN(Cc4c(OC)cc(-c5cn(C)c(=O)c6cnccc56)cc4OC)CC3)CC2)cc1N1CCC(=O)NC1=O] |
| B1 | 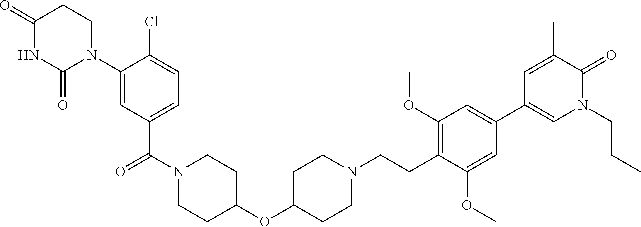 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)cc(C)c1=O] |
| B2 | 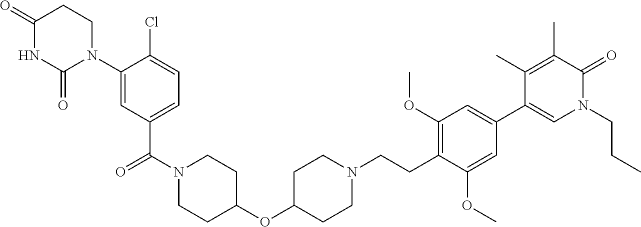 [SMILES: CCCn1cc(-c2cc(OC)c(CCN3CCC(OC4CCN(C(=O)c5ccc(Cl)c(N6CCC(=O)NC6=O)c5)CC4)CC3)c(OC)c2)c(C)c(C)c1=O] |
| B3 | 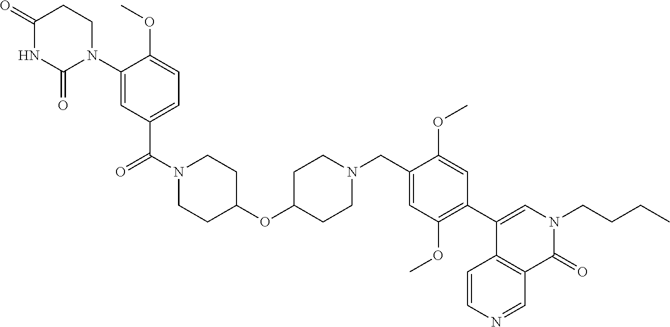 [SMILES: CCCCn1cc(-c2cc(OC)c(CN3CCC(OC4CCN(C(=O)c5ccc(OC)c(N6CCC(=O)NC6=O)c5)CC4)CC3)cc2OC)c2ccncc2c1=O] |
| B4 | 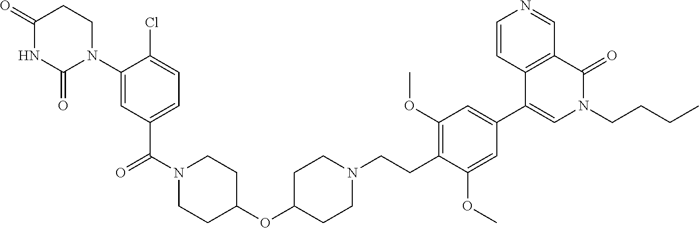 [SMILES: CCCCn1cc(-c2cc(OC)c(CCN3CCC(OC4CCN(C(=O)c5ccc(Cl)c(N6CCC(=O)NC6=O)c5)CC4)CC3)c(OC)c2)c2ccncc2c1=O] |
| B5 | 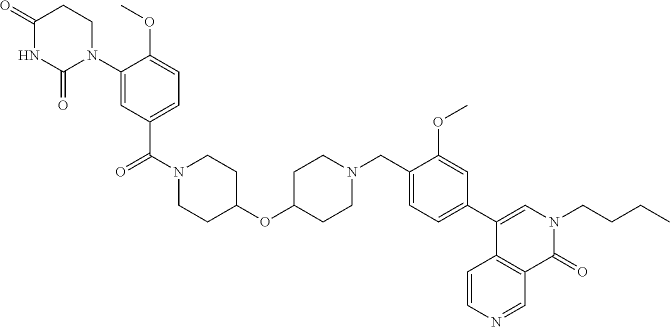 [SMILES: CCCCn1cc(-c2ccc(CN3CCC(OC4CCN(C(=O)c5ccc(OC)c(N6CCC(=O)NC6=O)c5)CC4)CC3)c(OC)c2)c2ccncc2c1=O] |
| B6 | 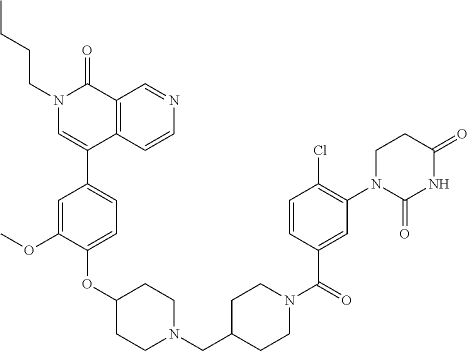 [SMILES: CCCCn1cc(-c2ccc(OC3CCN(CC4CCN(C(=O)c5ccc(Cl)c(N6CCC(=O)NC6=O)c5)CC4)CC3)c(OC)c2)c2ccncc2c1=O] |
| B7 | 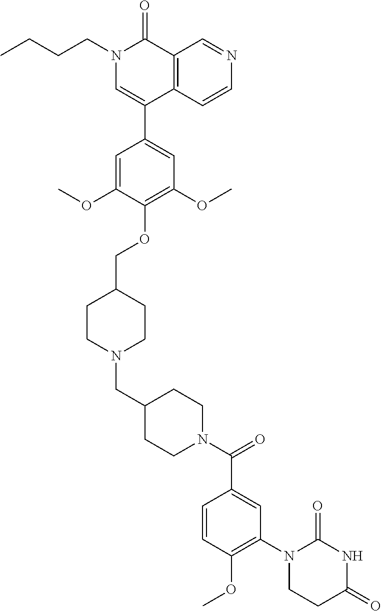 [SMILES: CCCCn1cc(-c2cc(OC)c(OCC3CCN(CC4CCN(C(=O)c5ccc(OC)c(N6CCC(=O)NC6=O)c5)CC4)CC3)c(OC)c2)c2ccncc2c1=O] |
| B8 | 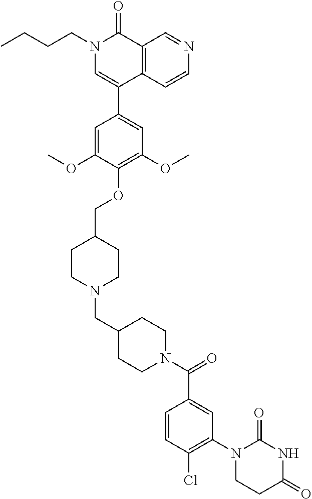 [SMILES: CCCCn1cc(-c2cc(OC)c(OCC3CCN(CC4CCN(C(=O)c5ccc(Cl)c(N6CCC(=O)NC6=O)c5)CC4)CC3)c(OC)c2)c2ccncc2c1=O] |
| B9 | 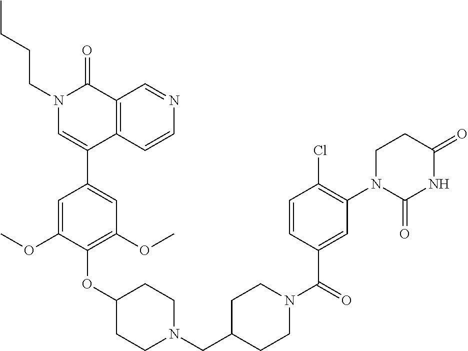 [SMILES: CCCCn1cc(-c2cc(OC)c(OC3CCN(CC4CCN(C(=O)c5ccc(Cl)c(N6CCC(=O)NC6=O)c5)CC4)CC3)c(OC)c2)c2ccncc2c1=O] |
| B10 | 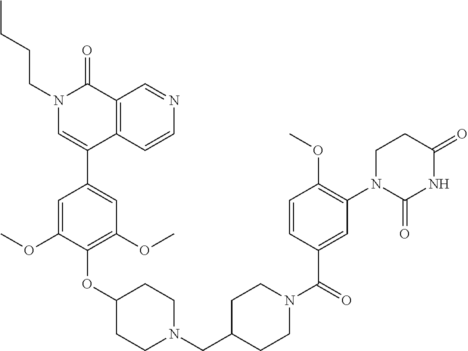 [SMILES: CCCCn1cc(-c2cc(OC)c(OC3CCN(CC4CCN(C(=O)c5ccc(OC)c(N6CCC(=O)NC6=O)c5)CC4)CC3)c(OC)c2)c2ccncc2c1=O] |
| B11 | 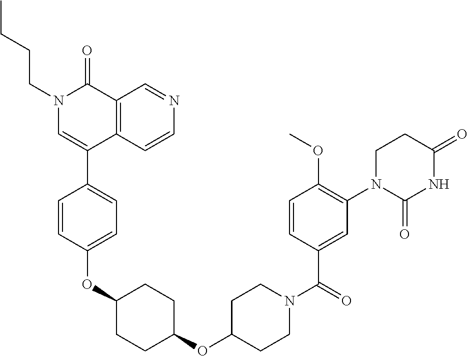 [SMILES: CCCCn1cc(-c2ccc(O[C@H]3CC[C@@H](OC4CCN(C(=O)c5ccc(OC)c(N6CCC(=O)NC6=O)c5)CC4)CC3)cc2)c2ccncc2c1=O] |
| B12 | 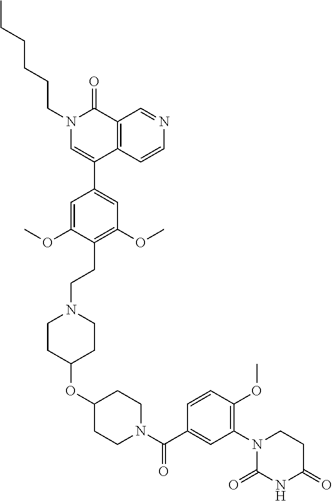 [SMILES: CCCCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] |
| B13 | 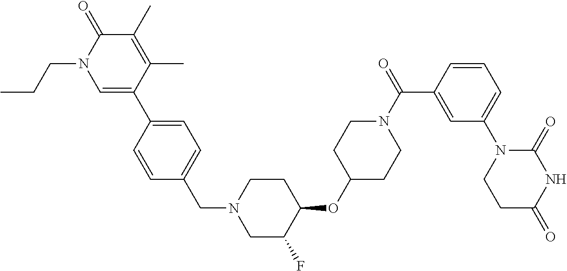 [SMILES: CCCn1cc(-c2ccc(CN3CC[C@@H](OC4CCN(C(=O)c5cccc(N6CCC(=O)NC6=O)c5)CC4)[C@H](F)C3)cc2)c(C)c(C)c1=O] |
| B14 | 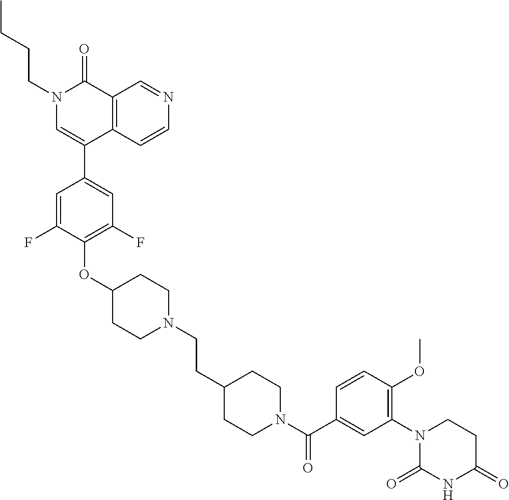 [SMILES: CCCCn1cc(-c2cc(F)c(OC3CCN(CCC4CCN(C(=O)c5ccc(OC)c(N6CCC(=O)NC6=O)c5)CC4)CC3)c(F)c2)c2ccncc2c1=O] |
| B15 | 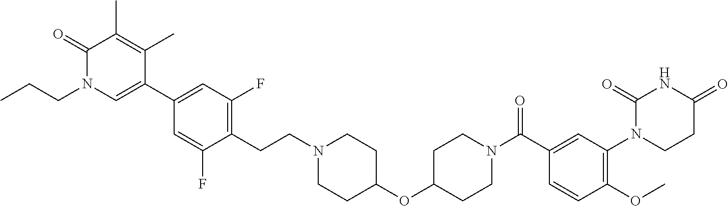 [SMILES: CCCn1cc(-c2cc(F)c(CCN3CCC(OC4CCN(C(=O)c5ccc(OC)c(N6CCC(=O)NC6=O)c5)CC4)CC3)c(F)c2)c(C)c(C)c1=O] |
| B16 | 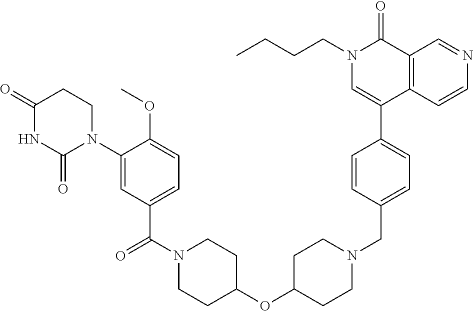 [SMILES: CCCCn1cc(-c2ccc(CN3CCC(OC4CCN(C(=O)c5ccc(OC)c(N6CCC(=O)NC6=O)c5)CC4)CC3)cc2)c2ccncc2c1=O] |
| B17 | 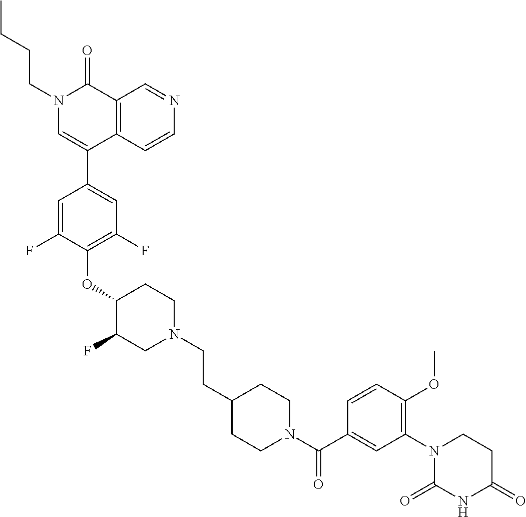 [SMILES: CCCCn1cc(-c2cc(F)c(O[C@@H]3CCN(CCC4CCN(C(=O)c5ccc(OC)c(N6CCC(=O)NC6=O)c5)CC4)C[C@H]3F)c(F)c2)c2ccncc2c1=O] |
| B18 | 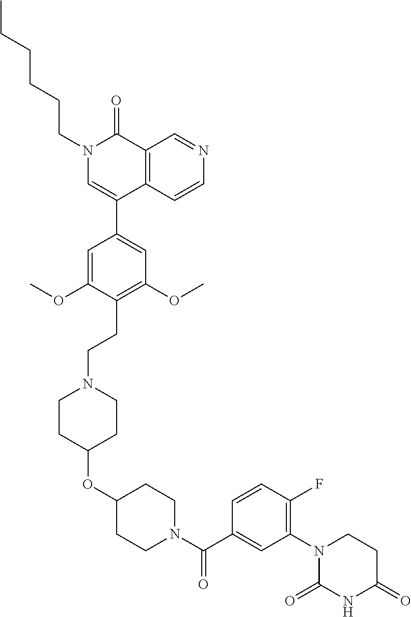 [SMILES: CCCCCCn1cc(-c2cc(OC)c(CCN3CCC(OC4CCN(C(=O)c5ccc(F)c(N6CCC(=O)NC6=O)c5)CC4)CC3)c(OC)c2)c2ccncc2c1=O] |
| B19 | 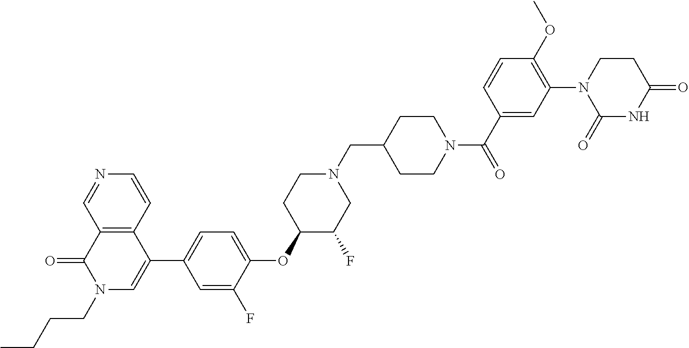 [SMILES: CCCCn1cc(-c2ccc(O[C@H]3CCN(CC4CCN(C(=O)c5ccc(OC)c(N6CCC(=O)NC6=O)c5)CC4)C[C@@H]3F)c(F)c2)c2ccncc2c1=O] |
| B20 | 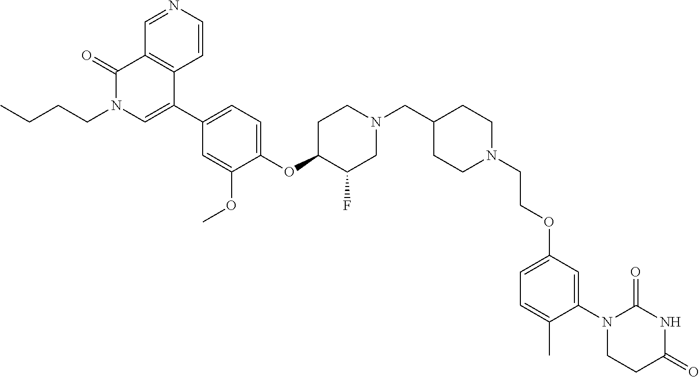 [SMILES: CCCCn1cc(-c2ccc(O[C@H]3CCN(CC4CCN(CCOc5ccc(C)c(N6CCC(=O)NC6=O)c5)CC4)C[C@@H]3F)c(OC)c2)c2ccncc2c1=O] |
| B22 | 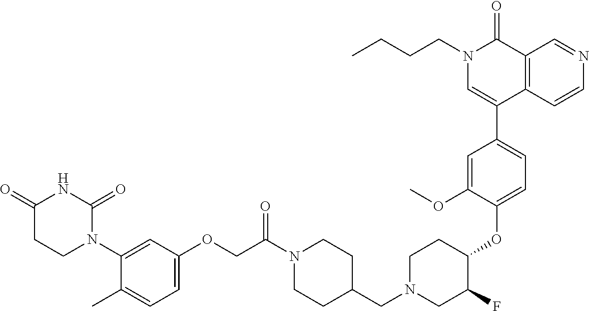 [SMILES: CCCCn1cc(-c2ccc(O[C@H]3CCN(CC4CCN(C(=O)COc5ccc(C)c(N6CCC(=O)NC6=O)c5)CC4)C[C@@H]3F)c(OC)c2)c2ccncc2c1=O] |
| B23 | 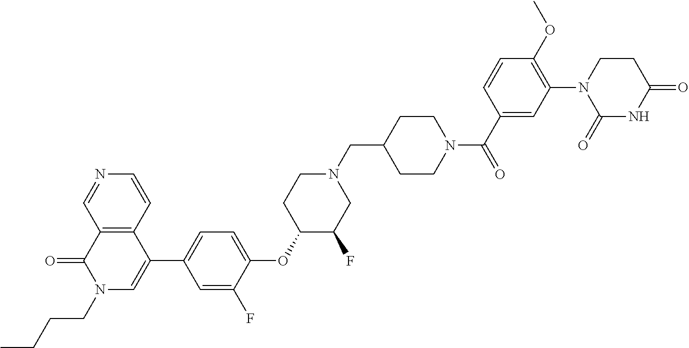 [SMILES: CCCCn1cc(-c2ccc(O[C@@H]3CCN(CC4CCN(C(=O)c5ccc(OC)c(N6CCC(=O)NC6=O)c5)CC4)C[C@H]3F)c(F)c2)c2ccncc2c1=O] |
| B24 | 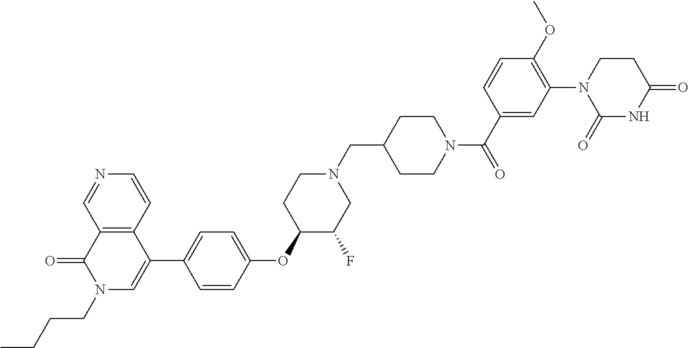 [SMILES: CCCCn1cc(-c2ccc(O[C@H]3CCN(CC4CCN(C(=O)c5ccc(OC)c(N6CCC(=O)NC6=O)c5)CC4)C[C@@H]3F)cc2)c2ccncc2c1=O] |
| B25 | 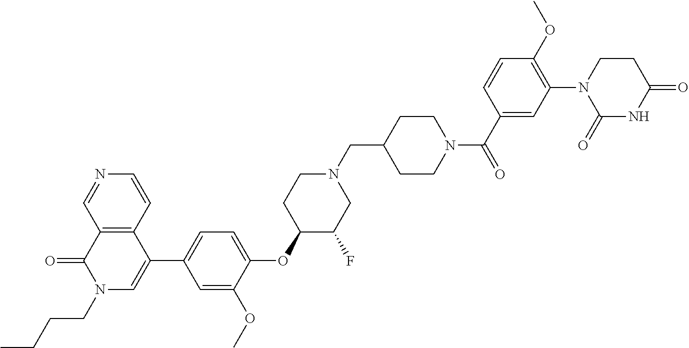 [SMILES: CCCCn1cc(-c2ccc(O[C@H]3CCN(CC4CCN(C(=O)c5ccc(OC)c(N6CCC(=O)NC6=O)c5)CC4)C[C@@H]3F)c(OC)c2)c2ccncc2c1=O] |
| B26 | 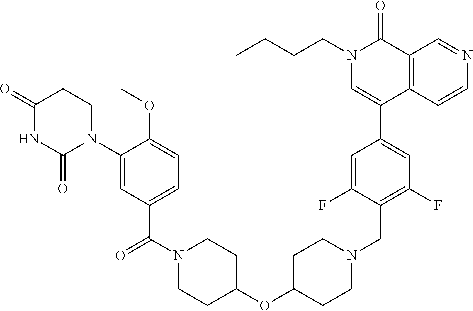 [SMILES: CCCCn1cc(-c2cc(F)c(CN3CCC(OC4CCN(C(=O)c5ccc(OC)c(N6CCC(=O)NC6=O)c5)CC4)CC3)c(F)c2)c2ccncc2c1=O] |
| B27 | 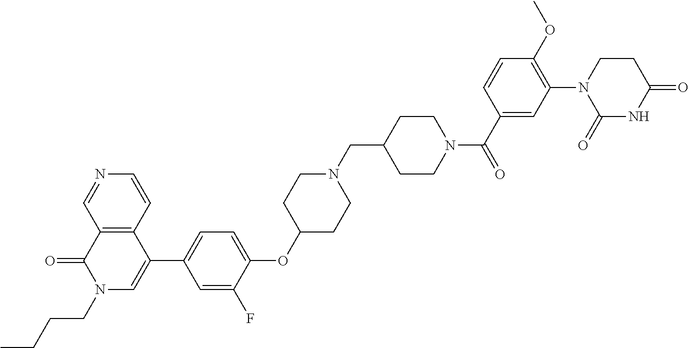 [SMILES: CCCCn1cc(-c2ccc(OC3CCN(CC4CCN(C(=O)c5ccc(OC)c(N6CCC(=O)NC6=O)c5)CC4)CC3)c(F)c2)c2ccncc2c1=O] |
| B28 | 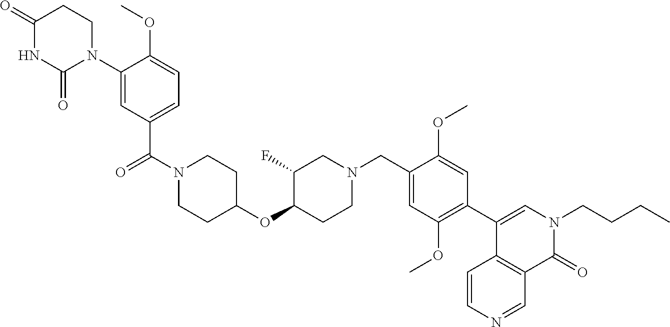 [SMILES: CCCCn1cc(-c2cc(OC)c(CN3CC[C@@H](OC4CCN(C(=O)c5ccc(OC)c(N6CCC(=O)NC6=O)c5)CC4)[C@H](F)C3)cc2OC)c2ccncc2c1=O] |
| B29 | 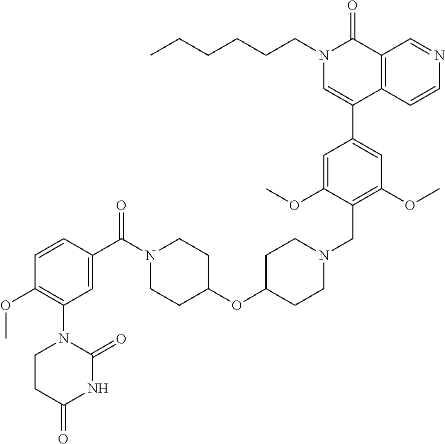 [SMILES: CCCCCCn1cc(-c2cc(OC)c(CN3CCC(OC4CCN(C(=O)c5ccc(OC)c(N6CCC(=O)NC6=O)c5)CC4)CC3)c(OC)c2)c2ccncc2c1=O] |
| B30 | 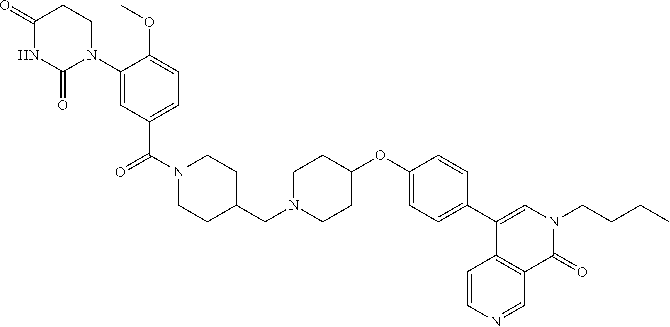 [SMILES: CCCCn1cc(-c2ccc(OC3CCN(CC4CCN(C(=O)c5ccc(OC)c(N6CCC(=O)NC6=O)c5)CC4)CC3)cc2)c2ccncc2c1=O] |
| B31 | 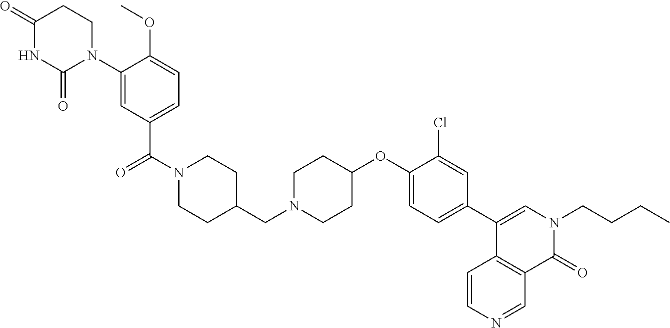 [SMILES: CCCCn1cc(-c2ccc(OC3CCN(CC4CCN(C(=O)c5ccc(OC)c(N6CCC(=O)NC6=O)c5)CC4)CC3)c(Cl)c2)c2ccncc2c1=O] |
| B32 | 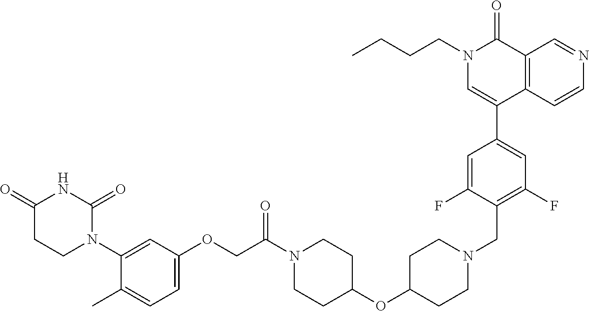 [SMILES: CCCCn1cc(-c2cc(F)c(CN3CCC(OC4CCN(C(=O)COc5ccc(C)c(N6CCC(=O)NC6=O)c5)CC4)CC3)c(F)c2)c2ccncc2c1=O] |
| B40 | 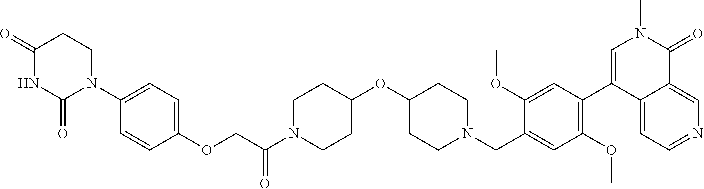 [SMILES: COc1cc(-c2cn(C)c(=O)c3cnccc23)c(OC)cc1CN1CCC(OC2CCN(C(=O)COc3ccc(N4CCC(=O)NC4=O)cc3)CC2)CC1] |
| B41 | 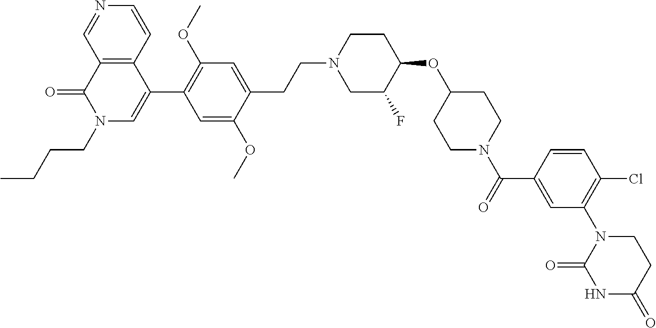 [SMILES: CCCCn1cc(-c2cc(OC)c(CCN3CC[C@@H](OC4CCN(C(=O)c5ccc(Cl)c(N6CCC(=O)NC6=O)c5)CC4)[C@H](F)C3)cc2OC)c2ccncc2c1=O] |
| B42 | 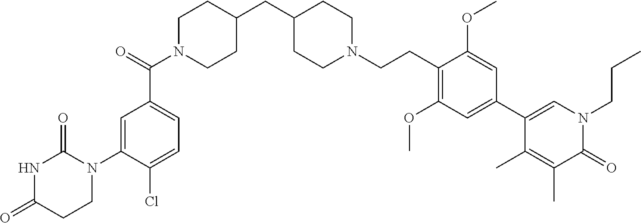 [SMILES: CCCn1cc(-c2cc(OC)c(CCN3CCC(CC4CCN(C(=O)c5ccc(Cl)c(N6CCC(=O)NC6=O)c5)CC4)CC3)c(OC)c2)c(C)c(C)c1=O] |
| B43 | 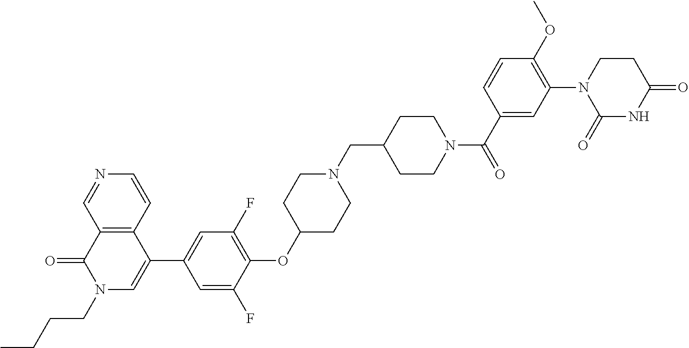 [SMILES: CCCCn1cc(-c2cc(F)c(OC3CCN(CC4CCN(C(=O)c5ccc(OC)c(N6CCC(=O)NC6=O)c5)CC4)CC3)c(F)c2)c2ccncc2c1=O] |
| B44 | 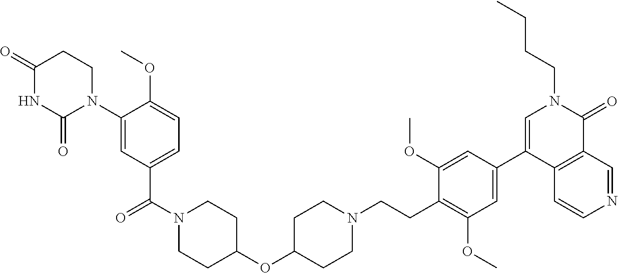 [SMILES: CCCCn1cc(-c2cc(OC)c(CCN3CCC(OC4CCN(C(=O)c5ccc(OC)c(N6CCC(=O)NC6=O)c5)CC4)CC3)c(OC)c2)c2ccncc2c1=O] |
| B45 | 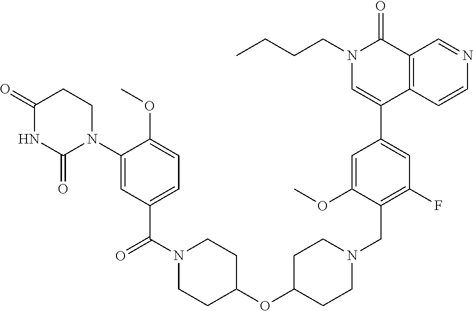 [SMILES: CCCCn1cc(-c2cc(F)c(CN3CCC(OC4CCN(C(=O)c5ccc(OC)c(N6CCC(=O)NC6=O)c5)CC4)CC3)c(OC)c2)c2ccncc2c1=O] |
| B46 | 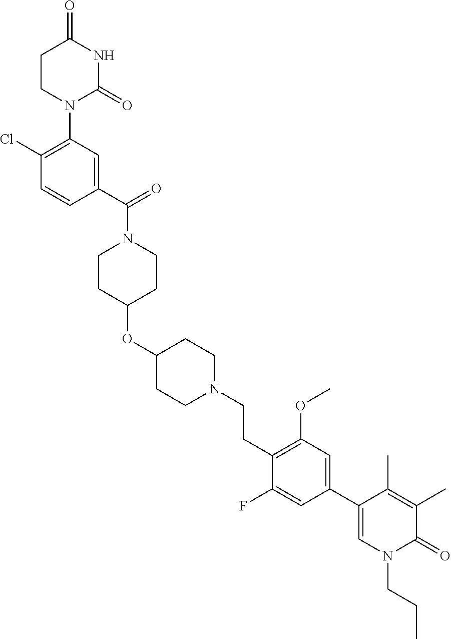 [SMILES: CCCn1cc(-c2cc(F)c(CCN3CCC(OC4CCN(C(=O)c5ccc(Cl)c(N6CCC(=O)NC6=O)c5)CC4)CC3)c(OC)c2)c(C)c(C)c1=O] |
| B47 | 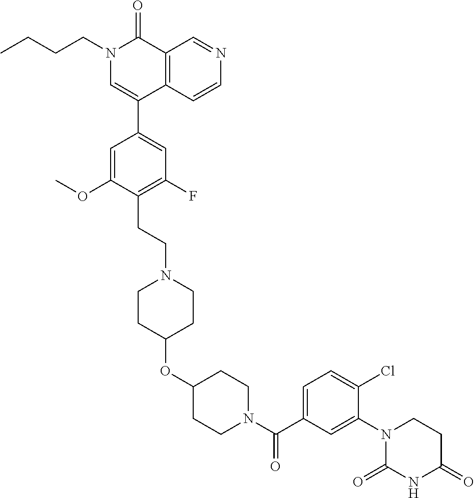 [SMILES: CCCCn1cc(-c2cc(F)c(CCN3CCC(OC4CCN(C(=O)c5ccc(Cl)c(N6CCC(=O)NC6=O)c5)CC4)CC3)c(OC)c2)c2ccncc2c1=O] |
| B48 | 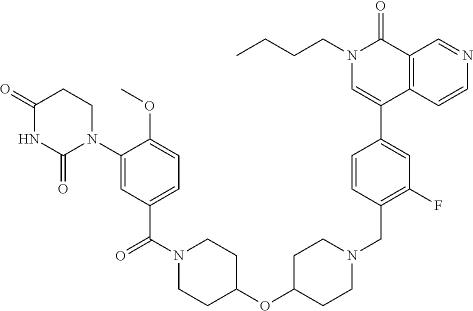 [SMILES: CCCCn1cc(-c2ccc(CN3CCC(OC4CCN(C(=O)c5ccc(OC)c(N6CCC(=O)NC6=O)c5)CC4)CC3)c(F)c2)c2ccncc2c1=O] |
| B49 | 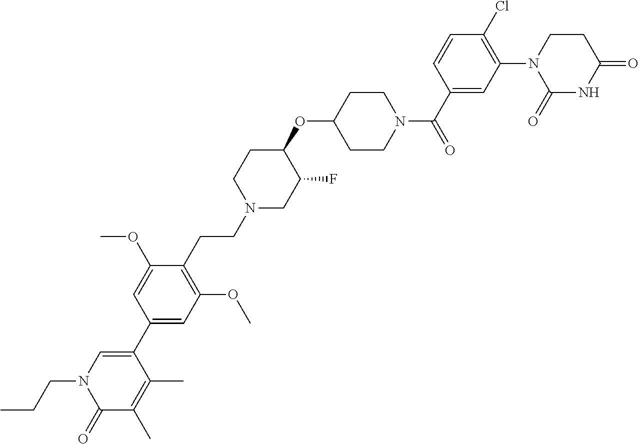 [SMILES: CCCn1cc(-c2cc(OC)c(CCN3CC[C@@H](OC4CCN(C(=O)c5ccc(Cl)c(N6CCC(=O)NC6=O)c5)CC4)[C@H](F)C3)c(OC)c2)c(C)c(C)c1=O] |
| B50 | 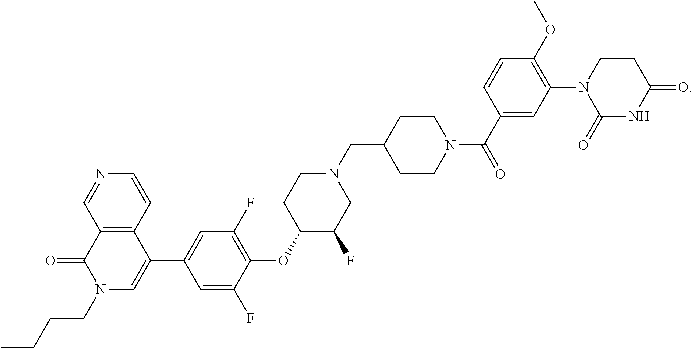 [SMILES: CCCCn1cc(-c2cc(F)c(O[C@@H]3CCN(CC4CCN(C(=O)c5ccc(OC)c(N6CCC(=O)NC6=O)c5)CC4)C[C@H]3F)c(F)c2)c2ccncc2c1=O] |
| C1 | 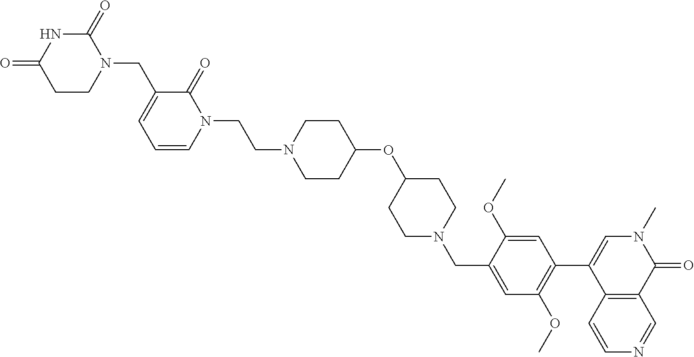 [SMILES: COc1cc(-c2cn(C)c(=O)c3cnccc23)c(OC)cc1CN1CCC(OC2CCN(CCn3cccc(CN4CCC(=O)NC4=O)c3=O)CC2)CC1] |
| C2 | 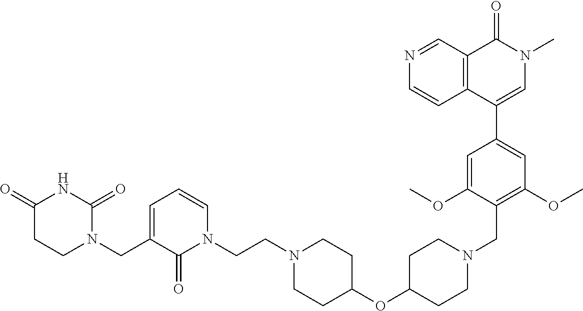 [SMILES: COc1cc(-c2cn(C)c(=O)c3cnccc23)cc(OC)c1CN1CCC(OC2CCN(CCn3cccc(CN4CCC(=O)NC4=O)c3=O)CC2)CC1] |
| C3 | 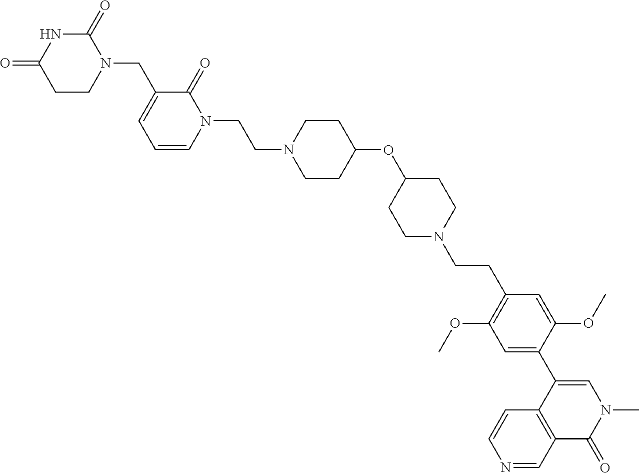 [SMILES: COc1cc(-c2cn(C)c(=O)c3cnccc23)c(OC)cc1CCN1CCC(OC2CCN(CCn3cccc(CN4CCC(=O)NC4=O)c3=O)CC2)CC1] |
| C4 | 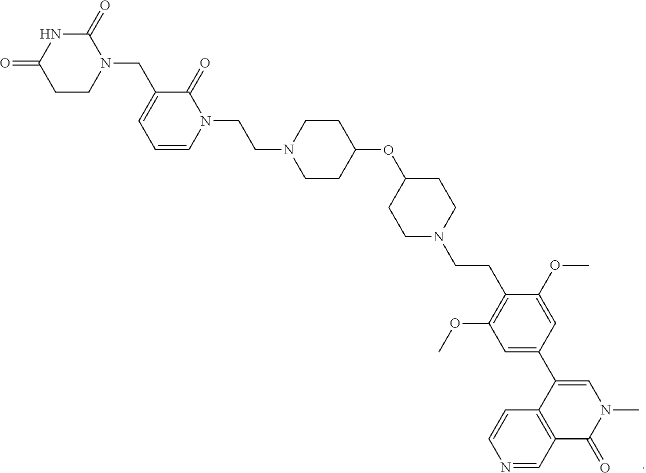 [SMILES: COc1cc(-c2cn(C)c(=O)c3cnccc23)cc(OC)c1CCN1CCC(OC2CCN(CCn3cccc(CN4CCC(=O)NC4=O)c3=O)CC2)CC1] |
| D1 | 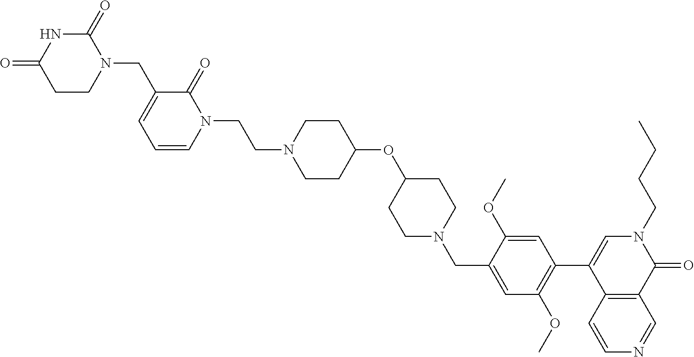 [SMILES: CCCCn1cc(-c2cc(OC)c(CN3CCC(OC4CCN(CCn5cccc(CN6CCC(=O)NC6=O)c5=O)CC4)CC3)cc2OC)c2ccncc2c1=O] |
| D2 | 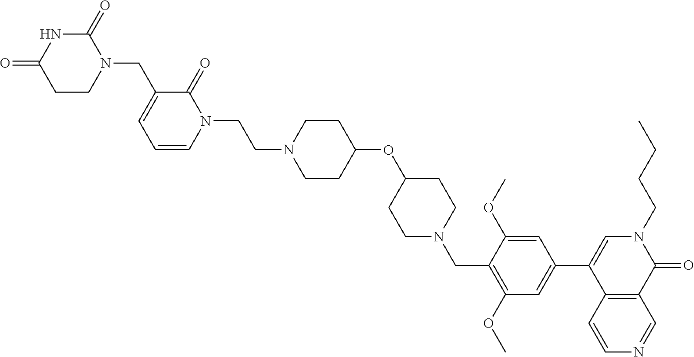 [SMILES: CCCCn1cc(-c2cc(OC)c(CN3CCC(OC4CCN(CCn5cccc(CN6CCC(=O)NC6=O)c5=O)CC4)CC3)c(OC)c2)c2ccncc2c1=O] |
| D3 | 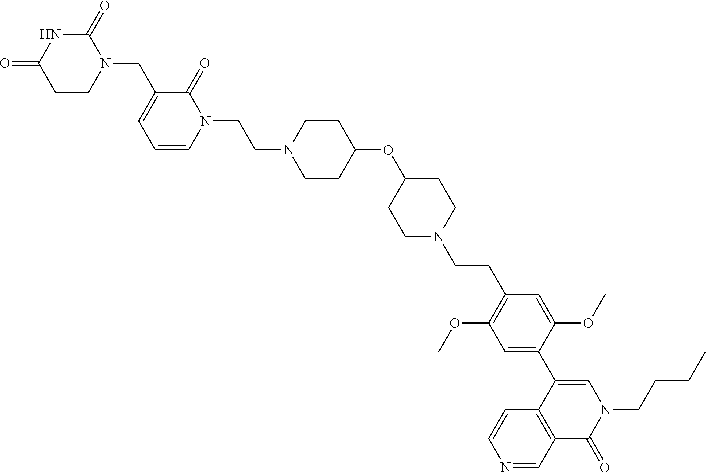 [SMILES: CCCCn1cc(-c2cc(OC)c(CCN3CCC(OC4CCN(CCn5cccc(CN6CCC(=O)NC6=O)c5=O)CC4)CC3)cc2OC)c2ccncc2c1=O] |
| D4 | 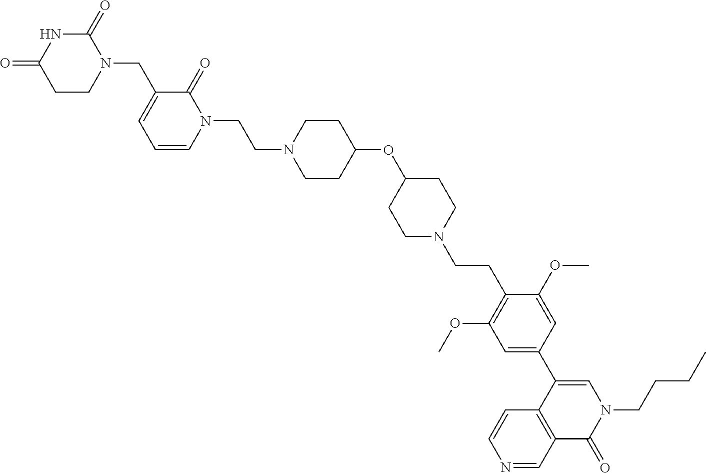 [SMILES: CCCCn1cc(-c2cc(OC)c(CCN3CCC(OC4CCN(CCn5cccc(CN6CCC(=O)NC6=O)c5=O)CC4)CC3)c(OC)c2)c2ccncc2c1=O] |
| E1 | 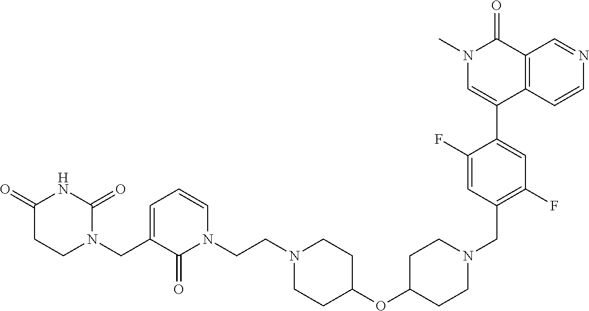 [SMILES: Cn1cc(-c2cc(F)c(CN3CCC(OC4CCN(CCn5cccc(CN6CCC(=O)NC6=O)c5=O)CC4)CC3)cc2F)c2ccncc2c1=O] |
| E2 | 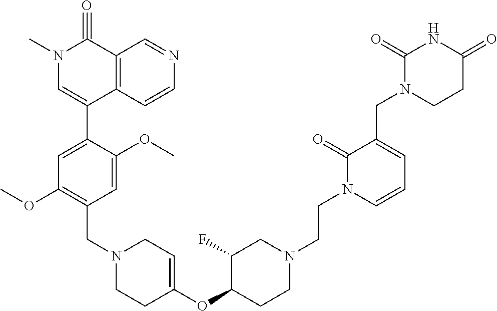 |
| E7 | 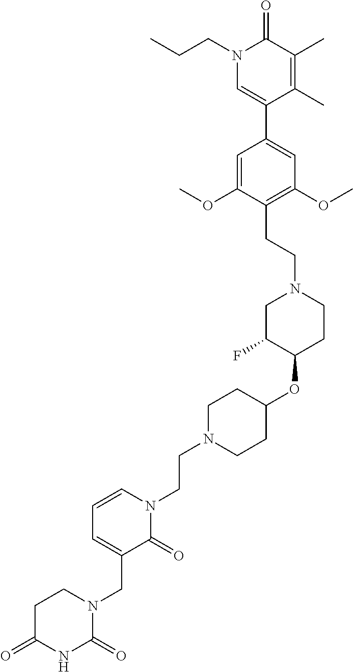 [SMILES: CCCn1cc(-c2cc(OC)c(CCN3CC[C@@H](OC4CCN(CCn5cccc(CN6CCC(=O)NC6=O)c5=O)CC4)[C@H](F)C3)c(OC)c2)c(C)c(C)c1=O] |
| E17 | 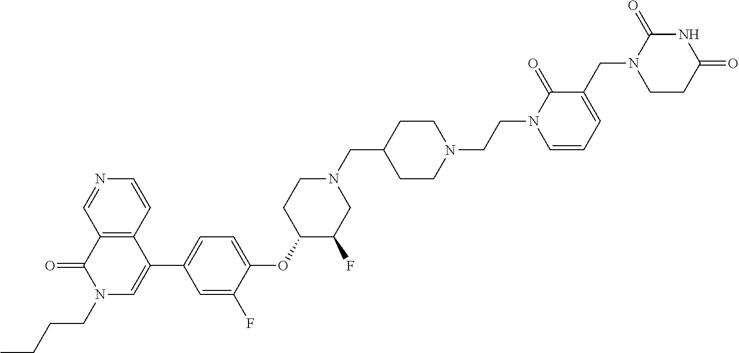 [SMILES: CCCCn1cc(-c2ccc(O[C@@H]3CCN(CC4CCN(CCn5cccc(CN6CCC(=O)NC6=O)c5=O)CC4)C[C@H]3F)c(F)c2)c2ccncc2c1=O] |
| E18 | 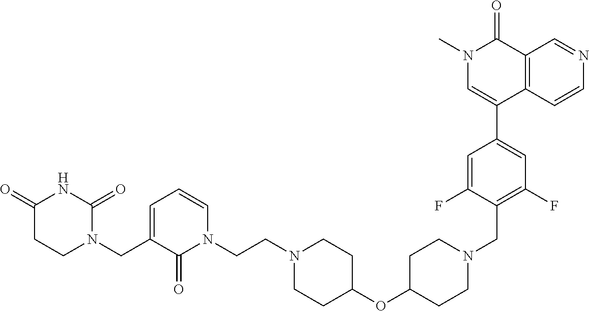 [SMILES: Cn1cc(-c2cc(F)c(CN3CCC(OC4CCN(CCn5cccc(CN6CCC(=O)NC6=O)c5=O)CC4)CC3)c(F)c2)c2ccncc2c1=O] |
| E23 | 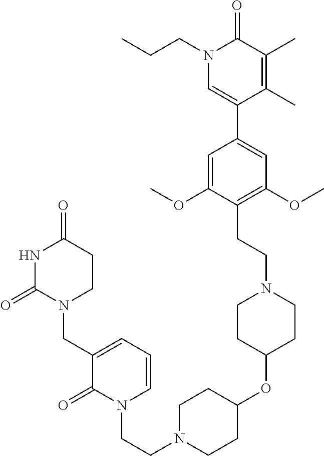 [SMILES: CCCn1cc(-c2cc(OC)c(CCN3CCC(OC4CCN(CCn5cccc(CN6CCC(=O)NC6=O)c5=O)CC4)CC3)c(OC)c2)c(C)c(C)c1=O] |
| E24 | 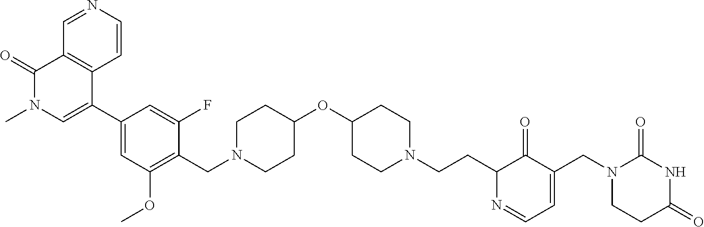 [SMILES: COc1cc(-c2cn(C)c(=O)c3cnccc23)cc(F)c1CN1CCC(OC2CCN(CCC3N=CC=C(CN4CCC(=O)NC4=O)C3=O)CC2)CC1] |
| E27 | 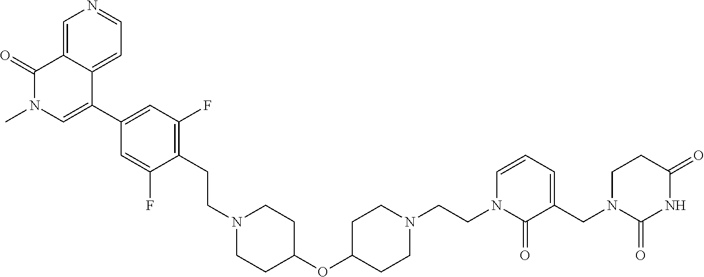 [SMILES: Cn1cc(-c2cc(F)c(CCN3CCC(OC4CCN(CCn5cccc(CN6CCC(=O)NC6=O)c5=O)CC4)CC3)c(F)c2)c2ccncc2c1=O] |
| E28 | 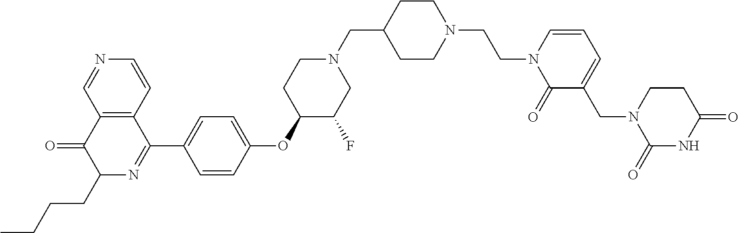 [SMILES: CCCCC1N=C(c2ccc(O[C@H]3CCN(CC4CCN(CCn5cccc(CN6CCC(=O)NC6=O)c5=O)CC4)C[C@@H]3F)cc2)c2ccncc2C1=O] |
| E30 | 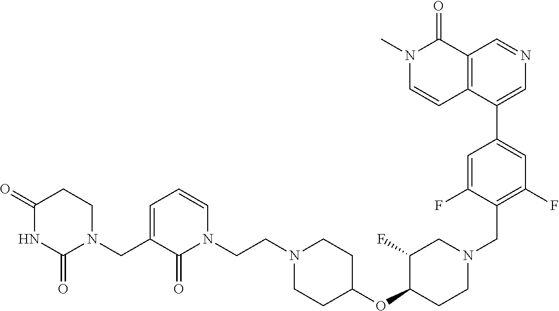 [SMILES: Cn1ccc2c(-c3cc(F)c(CN4CC[C@@H](OC5CCN(CCn6cccc(CN7CCC(=O)NC7=O)c6=O)CC5)[C@H](F)C4)c(F)c3)cncc2c1=O] |
| E35 | 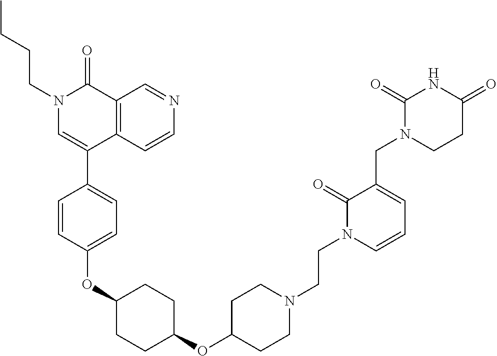 [SMILES: CCCCn1cc(-c2ccc(O[C@H]3CC[C@@H](OC4CCN(CCn5cccc(CN6CCC(=O)NC6=O)c5=O)CC4)CC3)cc2)c2ccncc2c1=O] |
| E37 | 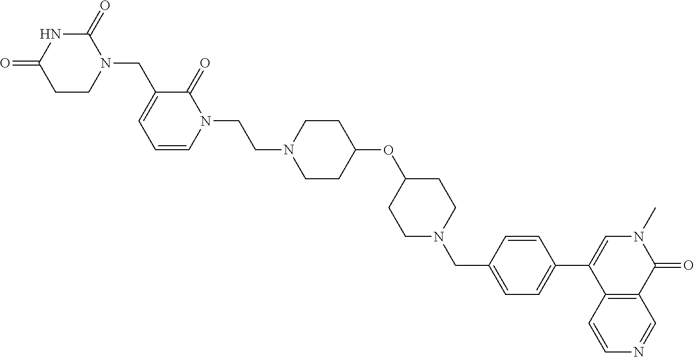 [SMILES: Cn1cc(-c2ccc(CN3CCC(OC4CCN(CCn5cccc(CN6CCC(=O)NC6=O)c5=O)CC4)CC3)cc2)c2ccncc2c1=O] and |
| E39 | 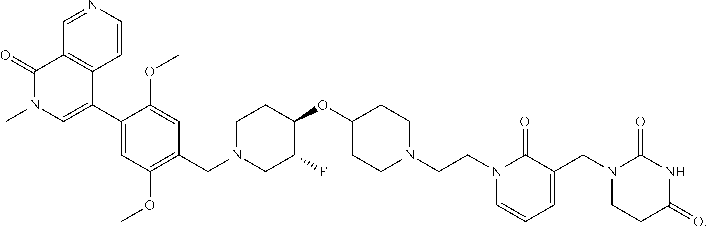 [SMILES: COc1cc(-c2cn(C)c(=O)c3cnccc23)c(OC)cc1CN1CC[C@@H](OC2CCN(CCn3cccc(CN4CCC(=O)NC4=O)c3=O)CC2)[C@H](F)C1] |

### WO2021178920 Compounds

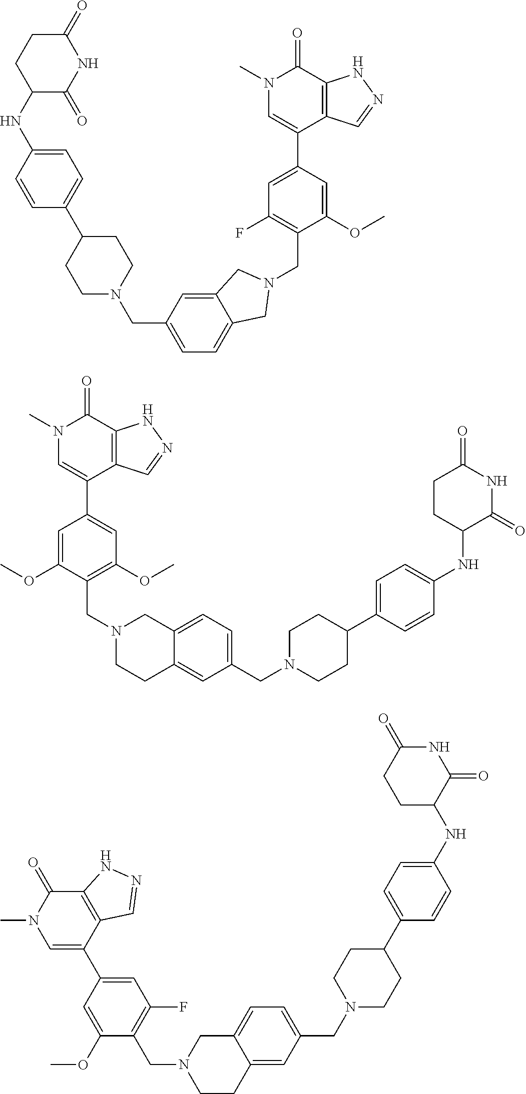

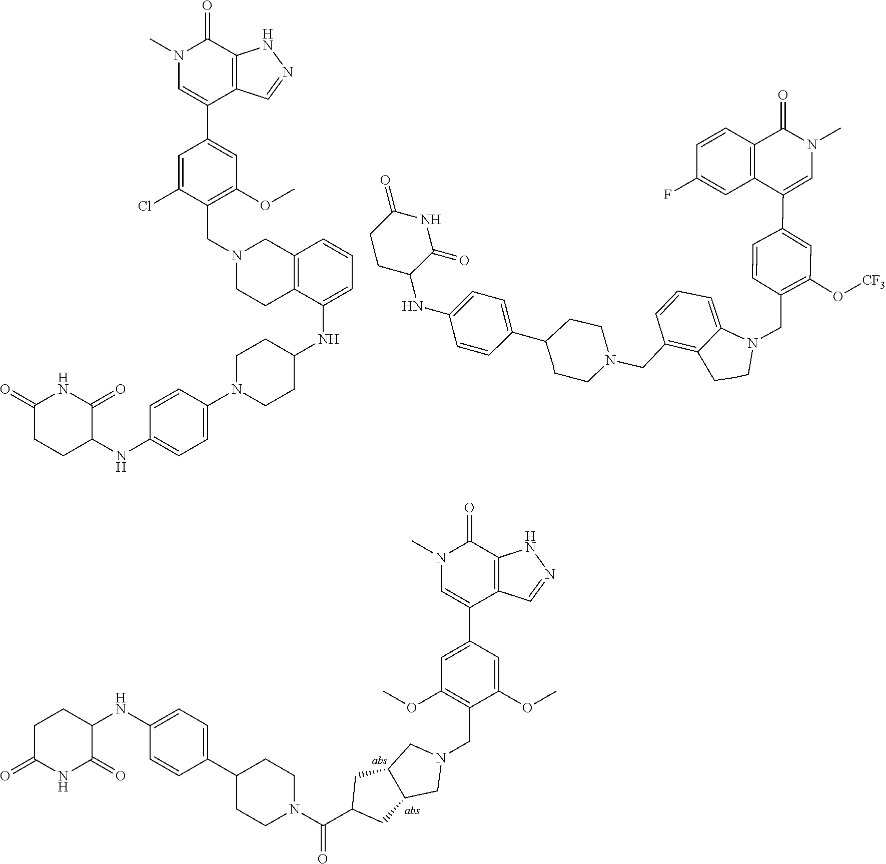

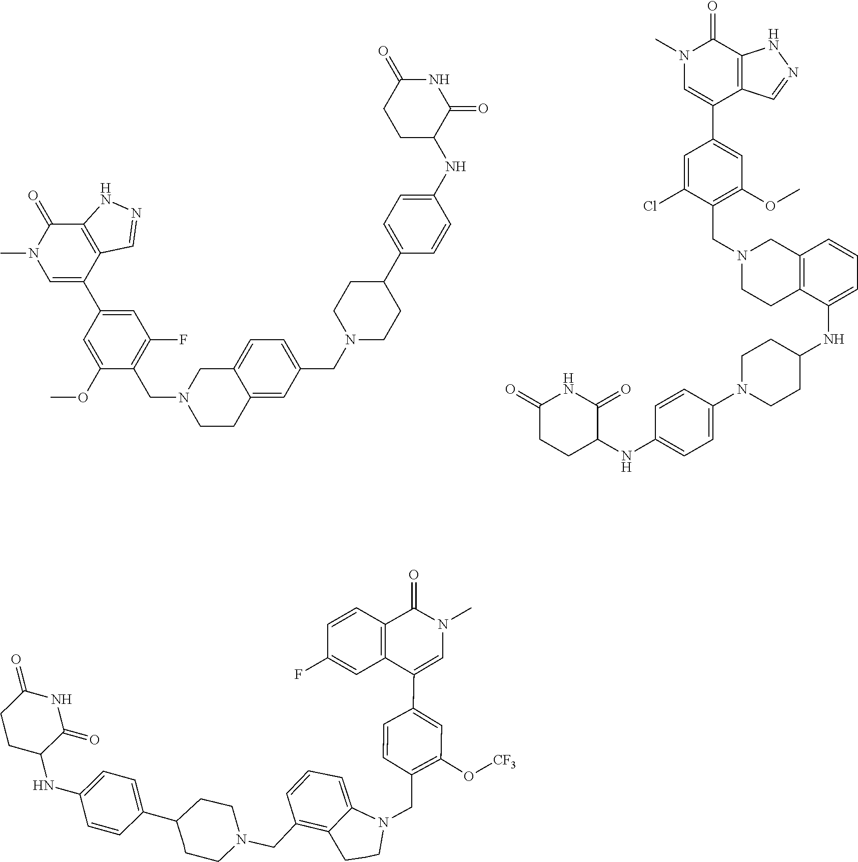 [SMILES: COc1cc(-c2cn(C)c(=O)c3[nH]ncc23)cc(F)c1CN1CCc2cc(CN3CCC(c4ccc(NC5CCC(=O)NC5=O)cc4)CC3)ccc2C1 ; COc1cc(-c2cn(C)c(=O)c3[nH]ncc23)cc(Cl)c1CN1CCc2c(cccc2NC2CCN(c3ccc(NC4CCC(=O)NC4=O)cc3)CC2)C1 ; Cn1cc(-c2ccc(CN3CCCc4c(CN5CCCC(c6ccc(NC7CCC(=O)NC7=O)cc6)CC5)cccc43)c(OC(F)(F)F)c2)c2cc(F)ccc2c1=O]

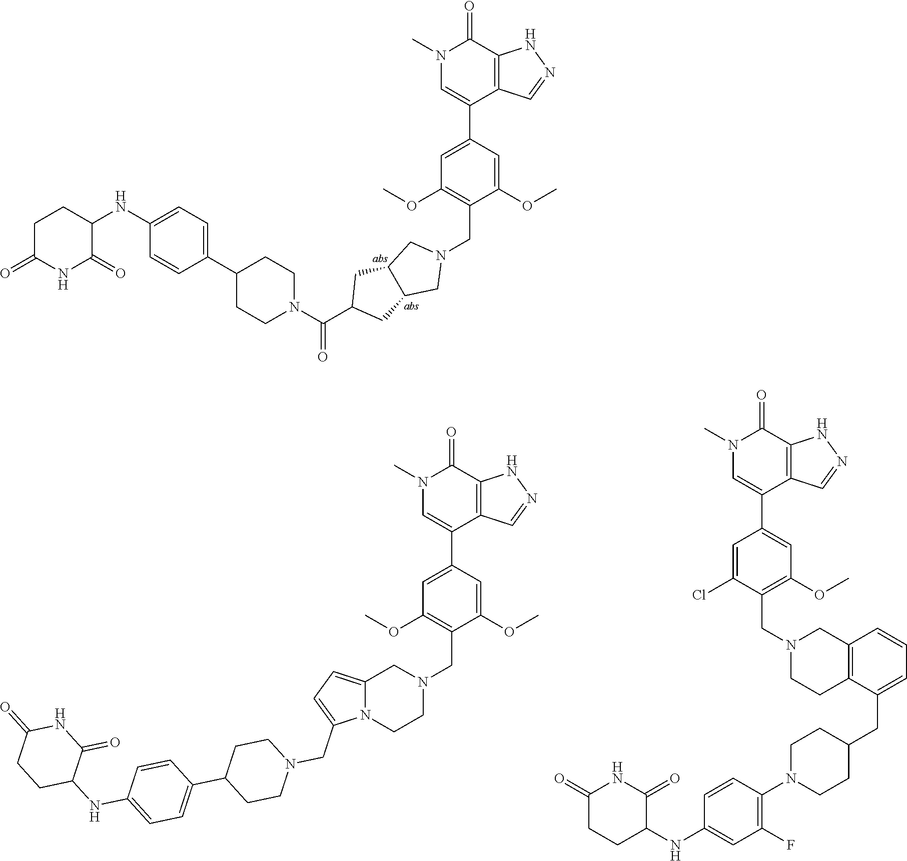

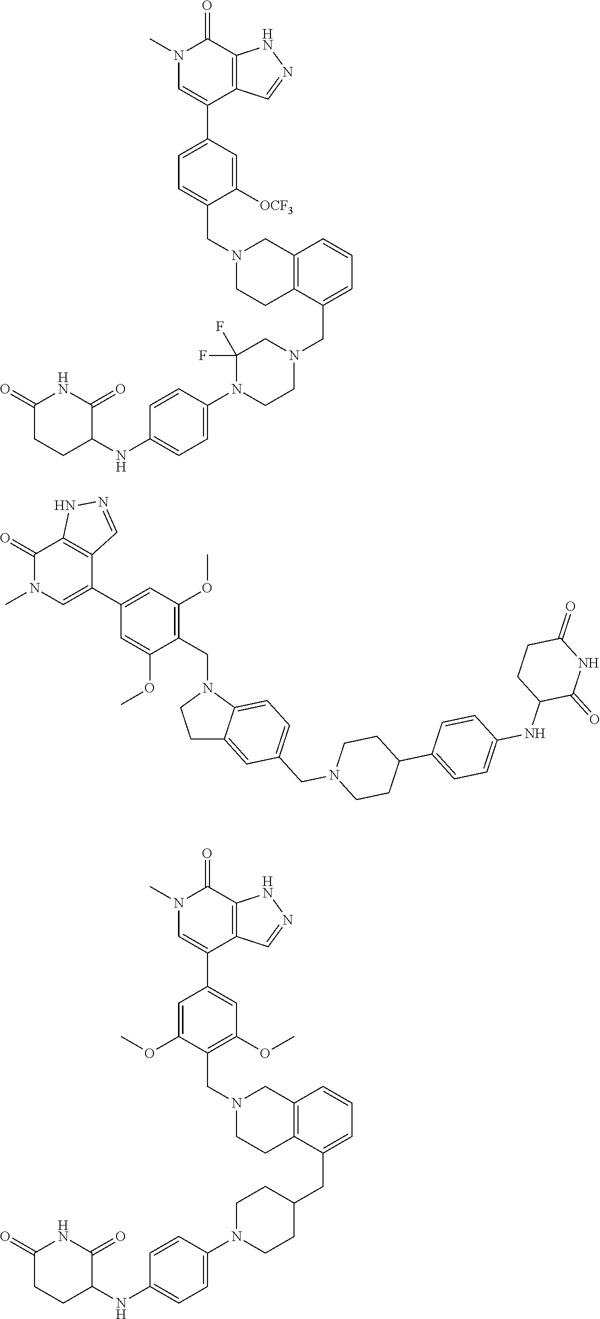 [SMILES: Cn1cc(-c2ccc(CN3CCc4c(cccc4CN4CCN(c5ccc(NC6CCC(=O)NC6=O)cc5)C(F)(F)C4)C3)c(OC(F)(F)F)c2)c2cn[nH]c2c1=O ; COc1cc(-c2cn(C)c(=O)c3[nH]ncc23)cc(OC)c1CN1CCc2cc(CN3CCC(c4ccc(NC5CCC(=O)NC5=O)cc4)CC3)ccc21 ; COc1cc(-c2cn(C)c(=O)c3[nH]ncc23)cc(OC)c1CN1CCc2c(CC3CCN(c4ccc(NC5CCC(=O)NC5=O)cc4)CC3)cccc2C1]

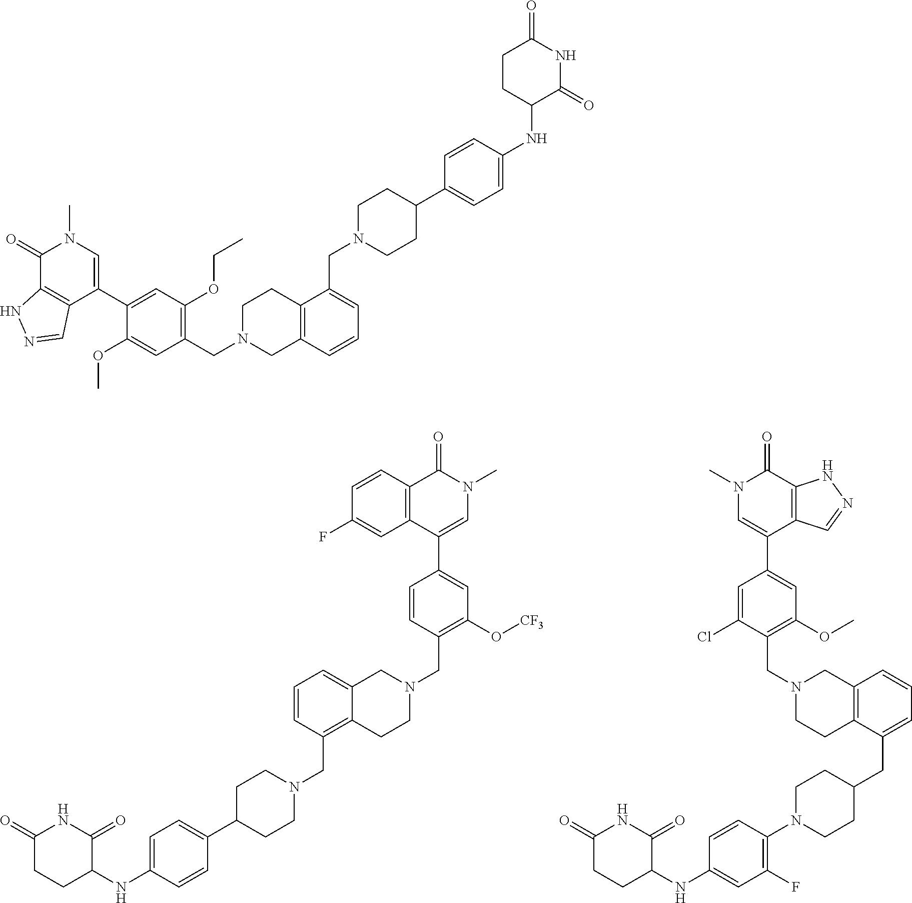 [SMILES: CCOc1cc(-c2cn(C)c(=O)c3[nH]ncc23)c(OC)cc1CN1CCc2c(CN3CCC(c4ccc(NC5CCC(=O)NC5=O)cc4)CC3)cccc2C1 ; Cn1cc(-c2ccc(CN3CCc4c(CN5CCC(c6ccc(NC7CCC(=O)NC7=O)cc6)CC5)cccc4C3)c(OC(F)(F)F)c2)c2cc(F)ccc2c1=O ; COc1cc(-c2cn(C)c(=O)c3[nH]ncc23)cc(Cl)c1CN1CCc2c(CC3CCN(c4ccc(NC5CCC(=O)NC5=O)cc4F)CC3)cccc2C1]

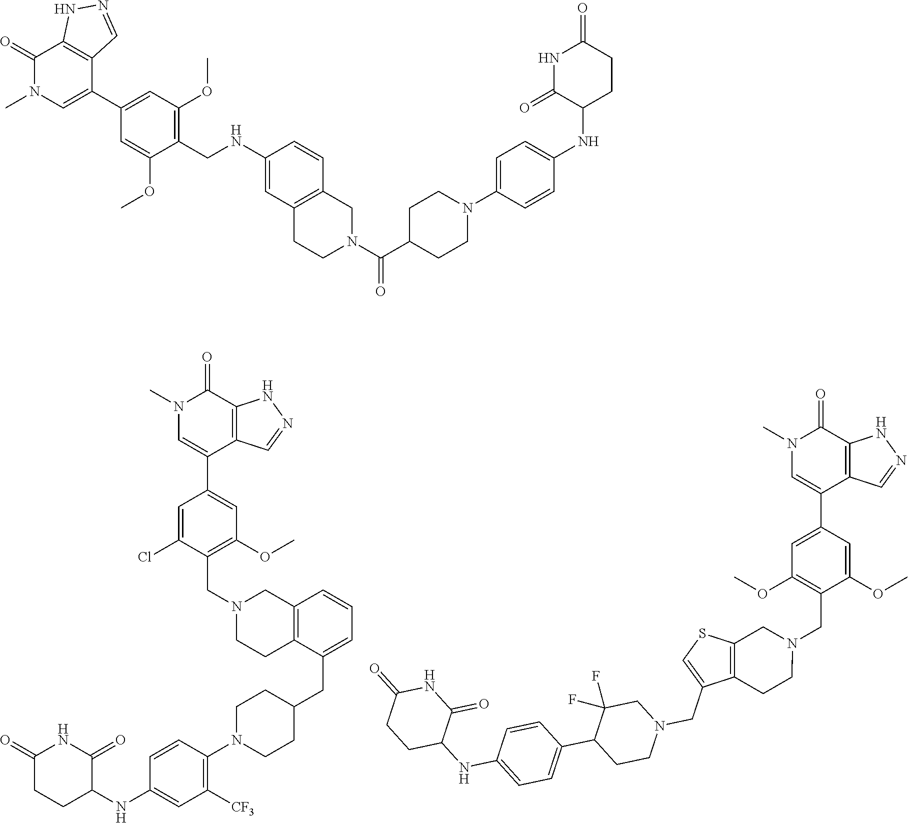

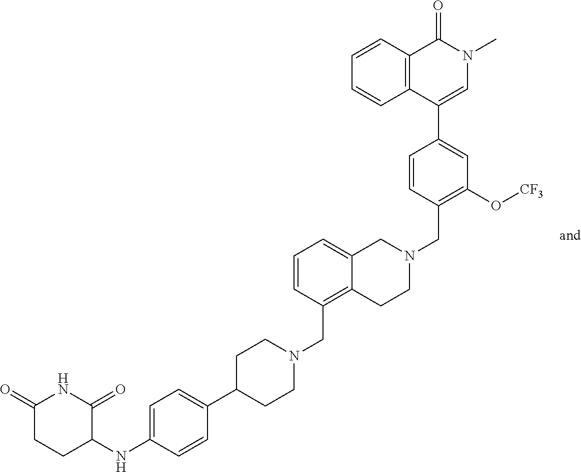 [SMILES: Cn1cc(-c2ccc(CN3CCc4c(CN5CCC(c6ccc(NC7CCC(=O)NC7=O)cc6)CC5)cccc4C3)c(OC(F)(F)F)c2)c2ccccc2c1=O]

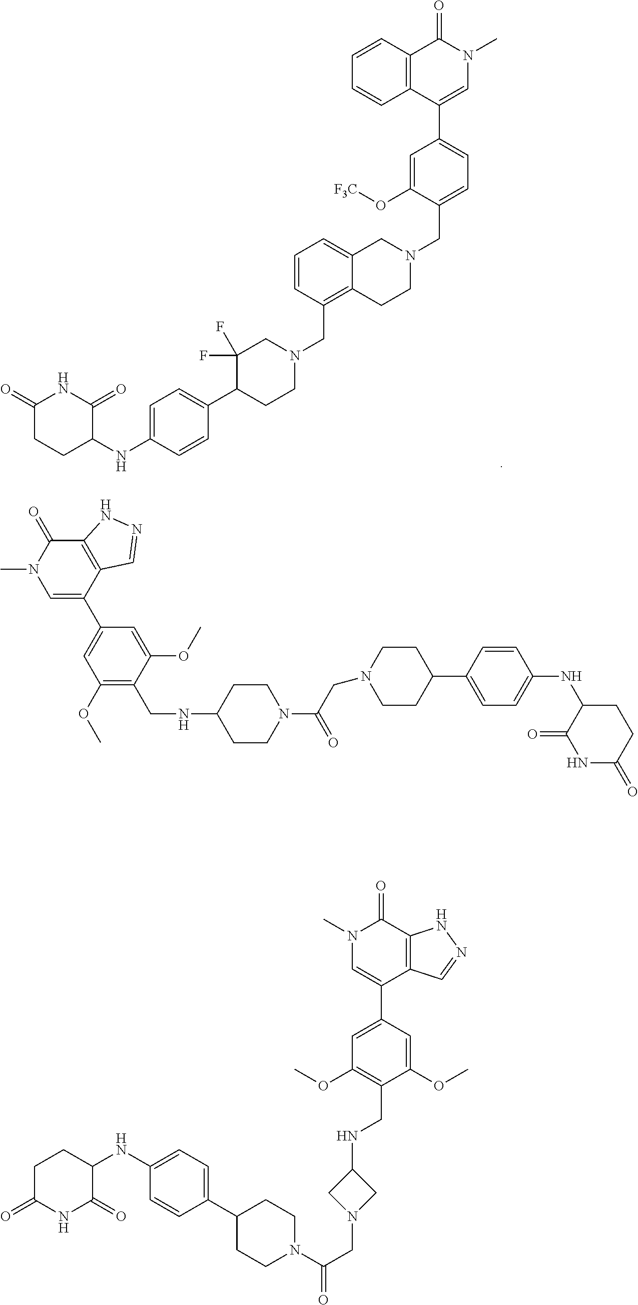

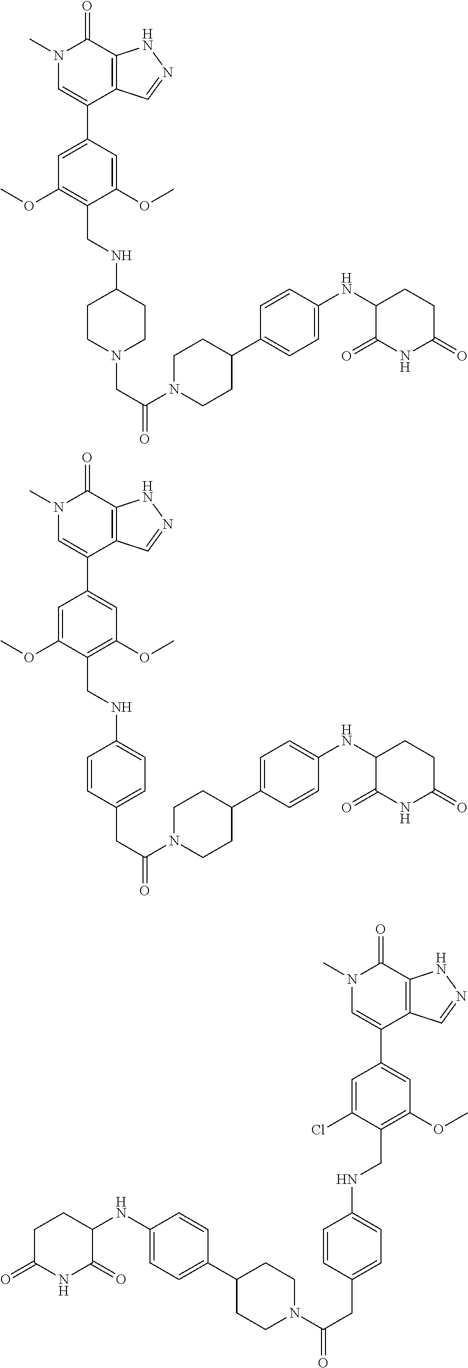 [SMILES: COc1cc(-c2cn(C)c(=O)c3[nH]ncc23)cc(OC)c1CNC1CCN(CC(=O)N2CCC(c3ccc(NC4CCC(=O)NC4=O)cc3)CC2)CC1 ; COc1cc(-c2cn(C)c(=O)c3[nH]ncc23)cc(OC)c1CNc1ccc(CC(=O)N2CCC(c3ccc(NC4CCC(=O)NC4=O)cc3)CC2)cc1 ; COc1cc(-c2cn(C)c(=O)c3[nH]ncc23)cc(Cl)c1CNc1ccc(CC(=O)N2CCC(c3ccc(NC4CCC(=O)NC4=O)cc3)CC2)cc1]

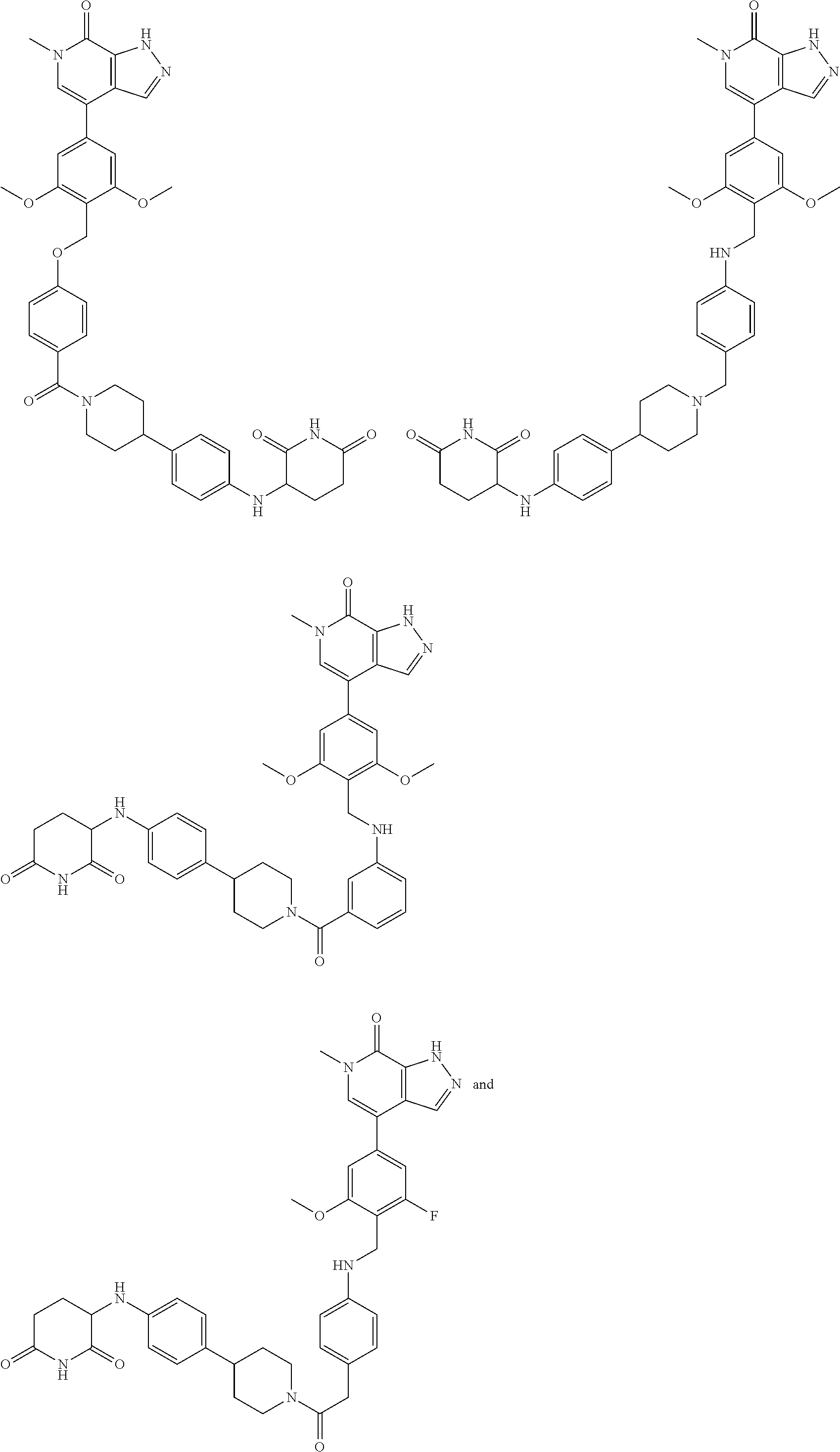

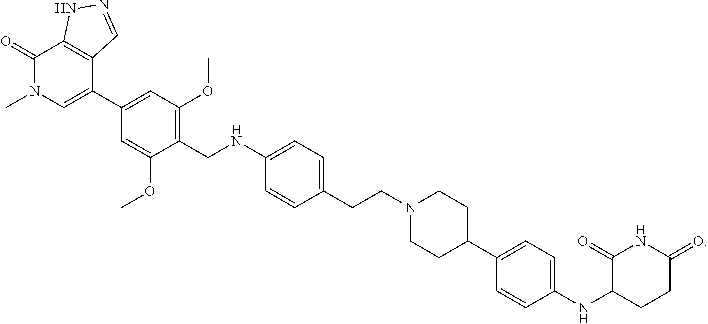 [SMILES: COc1cc(-c2cn(C)c(=O)c3[nH]ncc23)cc(OC)c1CNc1ccc(CCN2CCC(c3ccc(NC4CCC(=O)NC4=O)cc3)CC2)cc1]

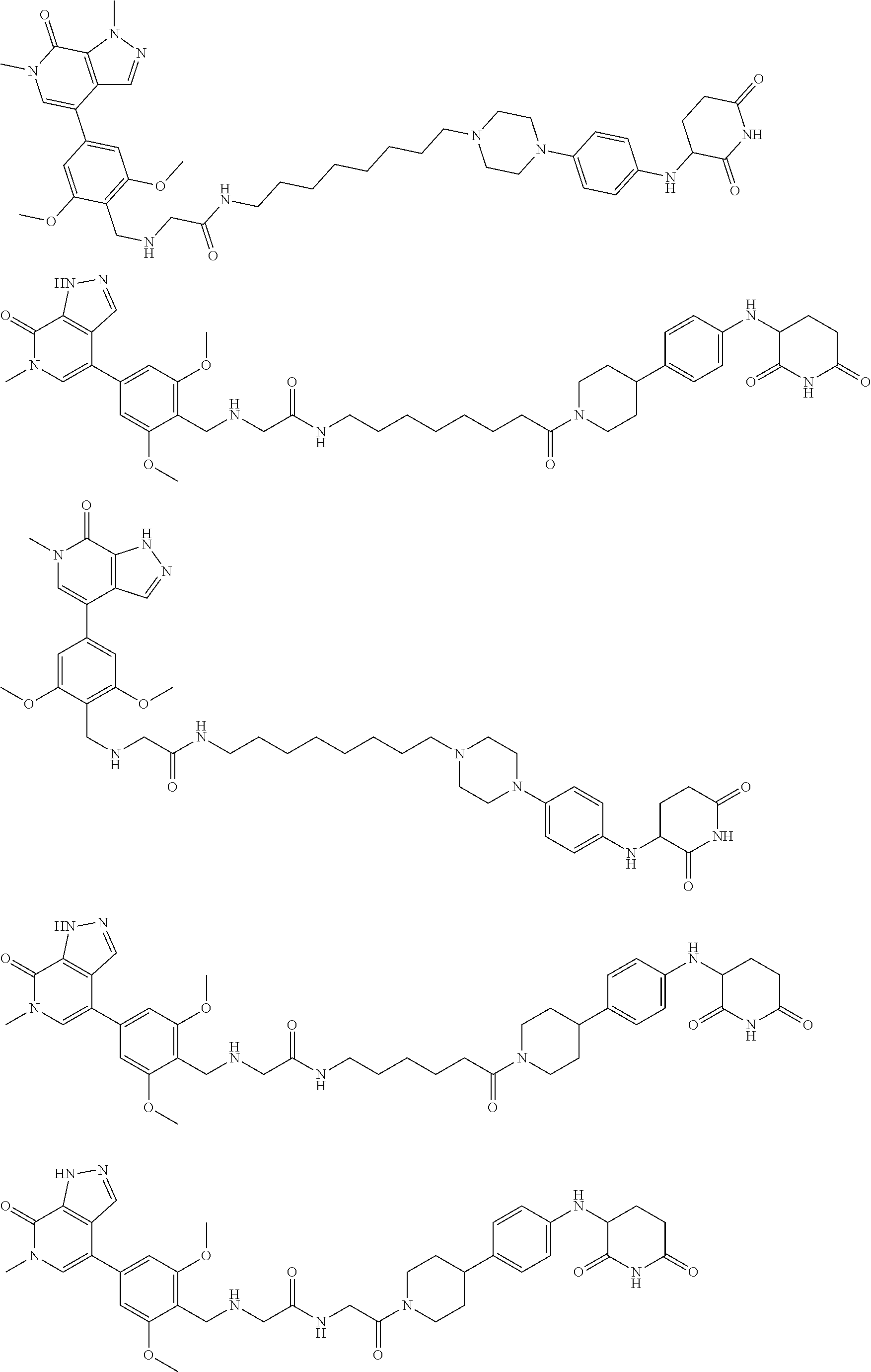 [SMILES: COc1cc(-c2cn(C)c(=O)c3c2cnn3C)cc(OC)c1CNCC(=O)NCCCCCCCCN1CCN(c2ccc(NC3CCC(=O)NC3=O)cc2)CC1 ; COc1cc(-c2cn(C)c(=O)c3[nH]ncc23)cc(OC)c1CNCC(=O)NCCCCCCCC(=O)N1CCC(c2ccc(NC3CCC(=O)NC3=O)cc2)CC1 ; COc1cc(-c2cn(C)c(=O)c3[nH]ncc23)cc(OC)c1CNCC(=O)NCCCCCCCCN1CCN(c2ccc(NC3CCC(=O)NC3=O)cc2)CC1 ; COc1cc(-c2cn(C)c(=O)c3[nH]ncc23)cc(OC)c1CNCC(=O)NCCCCCC(=O)N1CCC(c2ccc(NC3CCC(=O)NC3=O)cc2)CC1 ; COc1cc(-c2cn(C)c(=O)c3[nH]ncc23)cc(OC)c1CNCC(=O)NCC(=O)N1CCC(c2ccc(NC3CCC(=O)NC3=O)cc2)CC1]

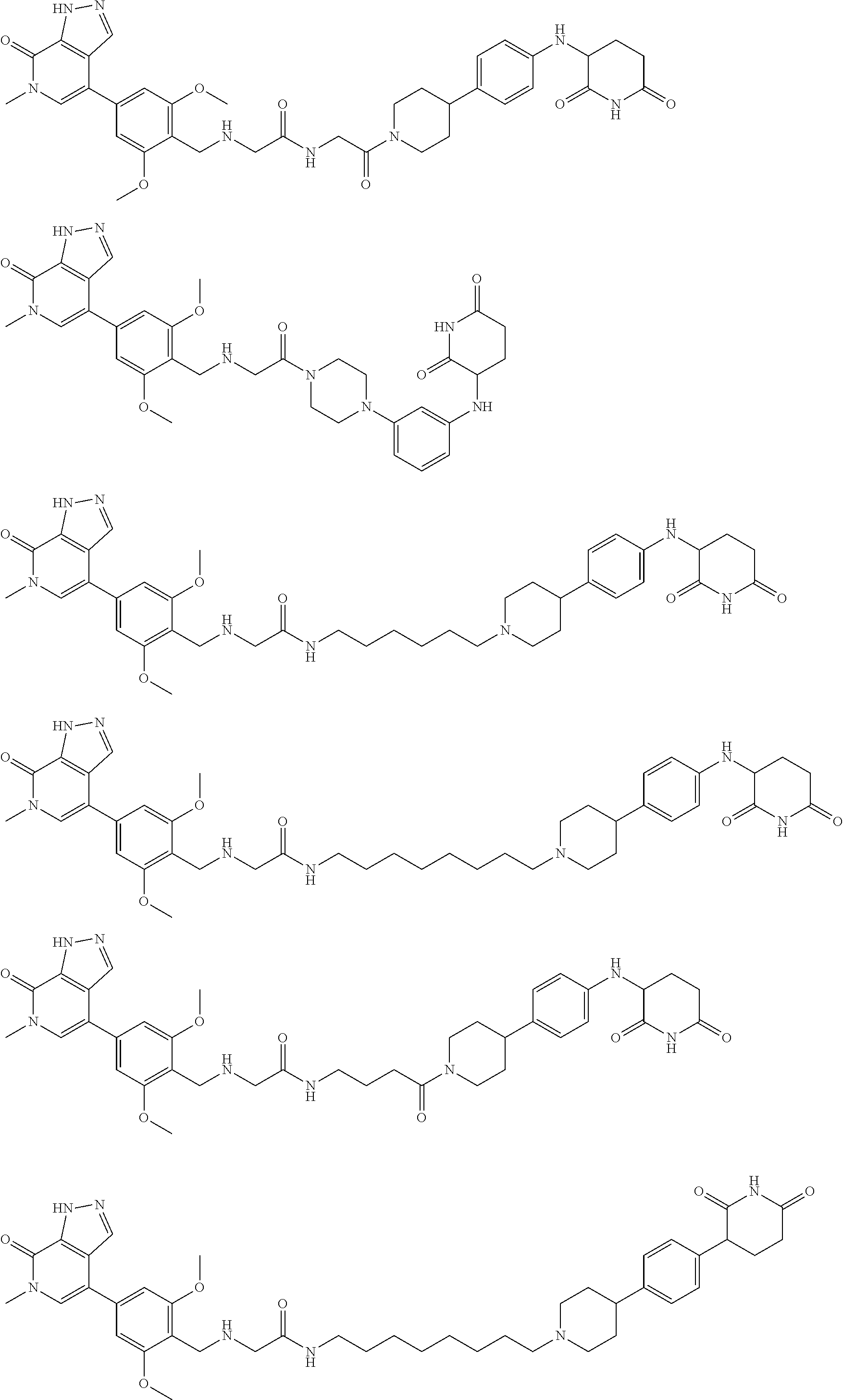

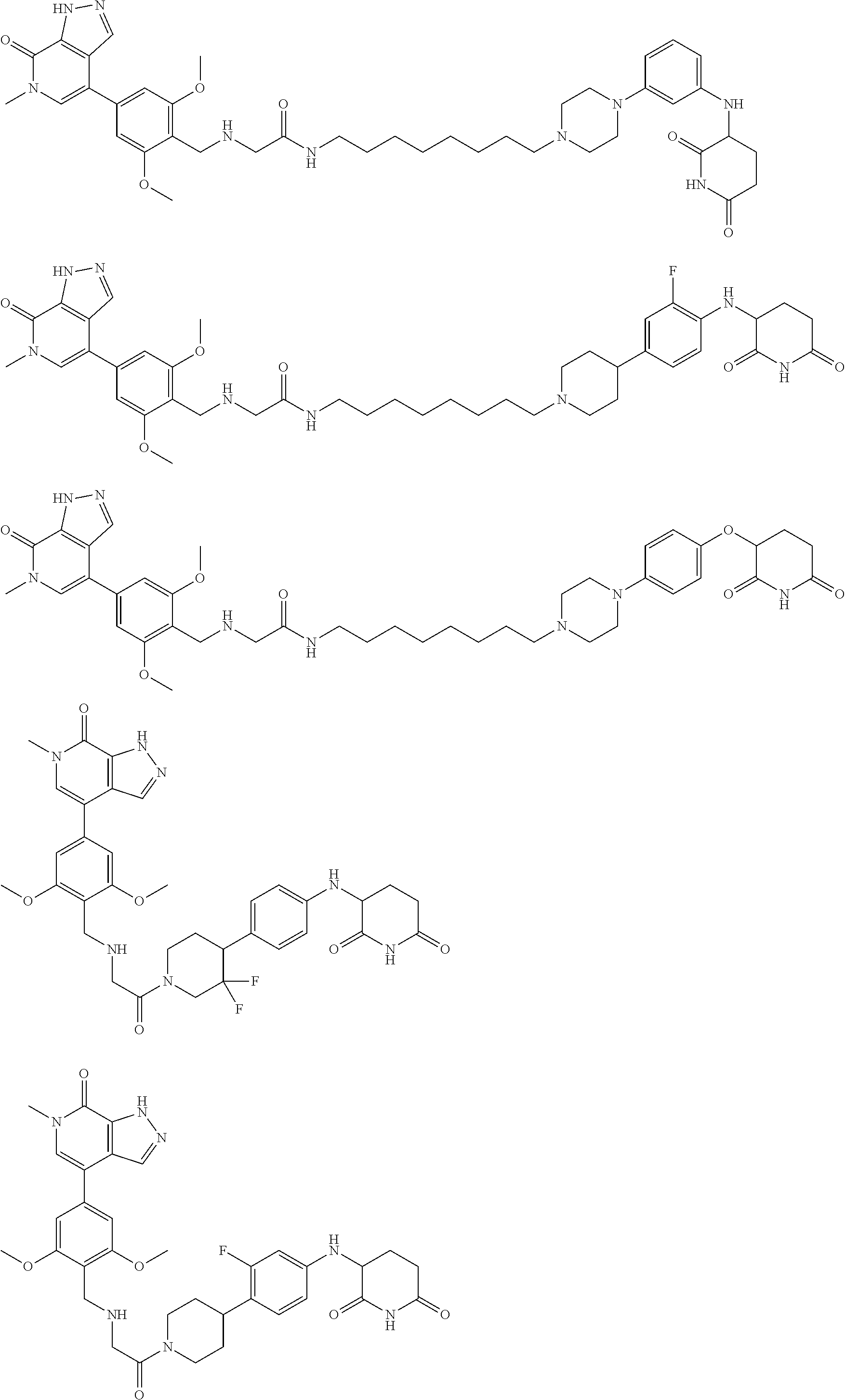 [SMILES: COc1cc(-c2cn(C)c(=O)c3[nH]ncc23)cc(OC)c1CNCC(=O)NCCCCCCCCN1CCN(c2cccc(NC3CCC(=O)NC3=O)c2)CC1 ; COc1cc(-c2cn(C)c(=O)c3[nH]ncc23)cc(OC)c1CNCC(=O)NCCCCCCCCN1CCC(c2ccc(NC3CCC(=O)NC3=O)c(F)c2)CC1 ; COc1cc(-c2cn(C)c(=O)c3[nH]ncc23)cc(OC)c1CNCC(=O)NCCCCCCCCN1CCN(c2ccc(OC3CCC(=O)NC3=O)cc2)CC1 ; COc1cc(-c2cn(C)c(=O)c3[nH]ncc23)cc(OC)c1CNCC(=O)N1CCC(c2ccc(NC3CCC(=O)NC3=O)cc2)C(F)(F)C1 ; COc1cc(-c2cn(C)c(=O)c3[nH]ncc23)cc(OC)c1CNCC(=O)N1CCC(c2ccc(NC3CCC(=O)NC3=O)cc2F)CC1]

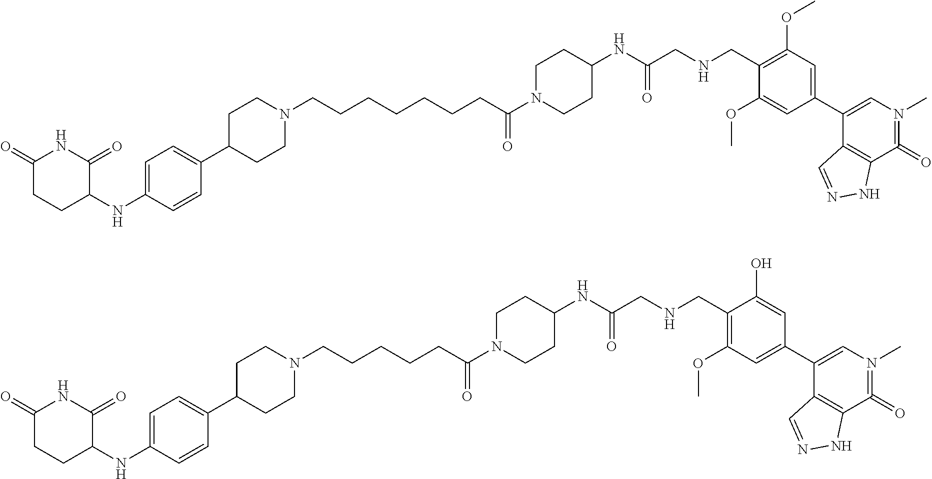

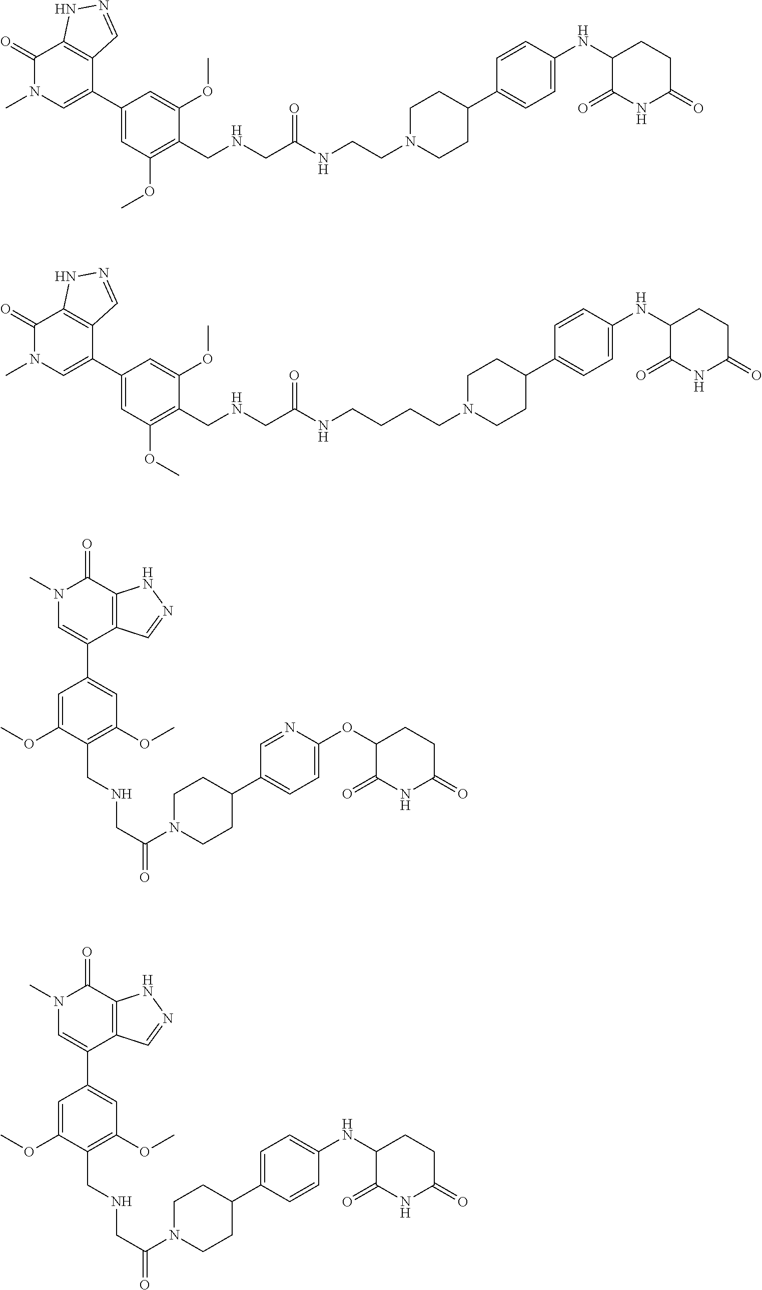 [SMILES: COc1cc(-c2cn(C)c(=O)c3[nH]ncc23)cc(OC)c1CNCC(=O)NCCN1CCC(c2ccc(NC3CCC(=O)NC3=O)cc2)CC1 ; COc1cc(-c2cn(C)c(=O)c3[nH]ncc23)cc(OC)c1CNCC(=O)NCCCCN1CCC(c2ccc(NC3CCC(=O)NC3=O)cc2)CC1 ; COc1cc(-c2cn(C)c(=O)c3[nH]ncc23)cc(OC)c1CNCC(=O)N1CCC(c2ccc(OC3CCC(=O)NC3=O)nc2)CC1 ; COc1cc(-c2cn(C)c(=O)c3[nH]ncc23)cc(OC)c1CNCC(=O)N1CCC(c2ccc(NC3CCC(=O)NC3=O)cc2)CC1]

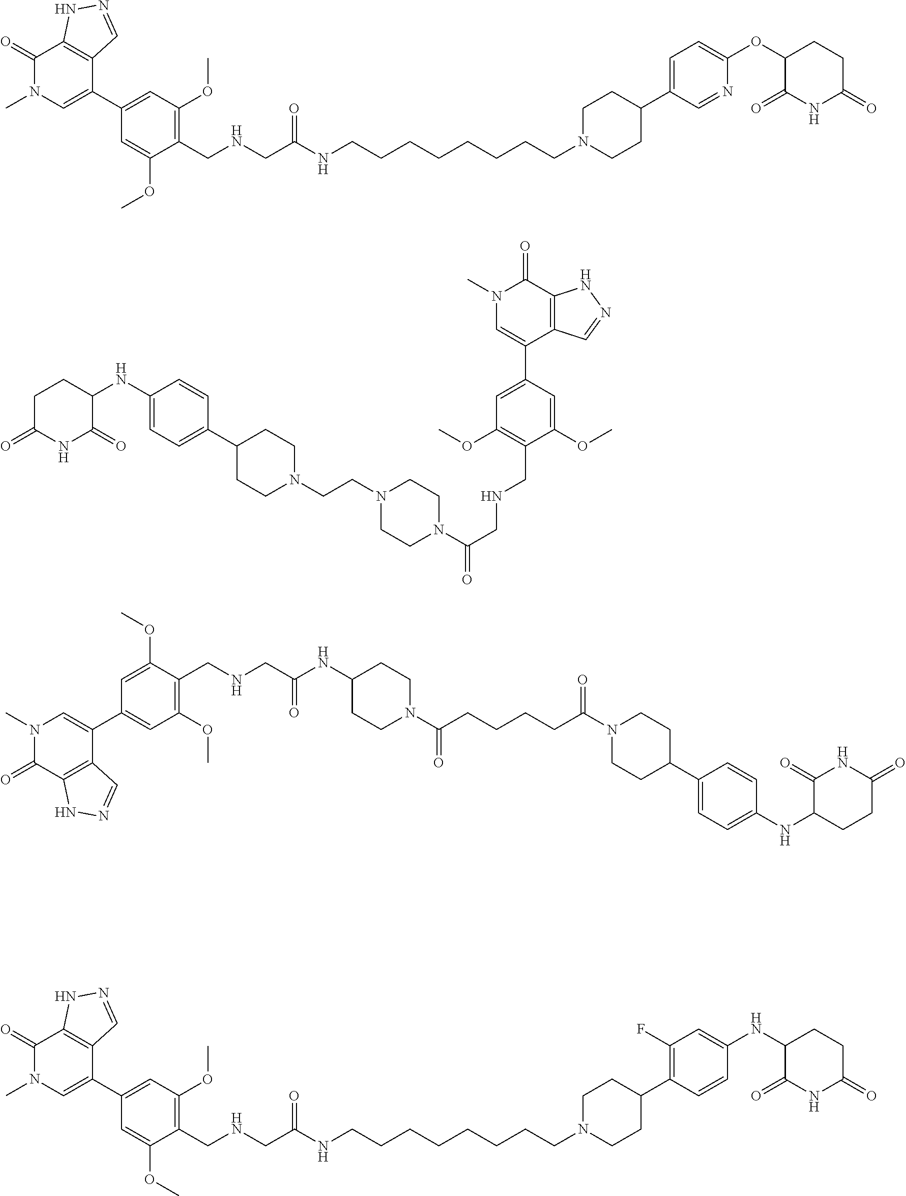 [SMILES: COc1cc(-c2cn(C)c(=O)c3[nH]ncc23)cc(OC)c1CNCC(=O)NCCCCCCCCN1CCC(c2ccc(OC3CCC(=O)NC3=O)nc2)CC1 ; COc1cc(-c2cn(C)c(=O)c3[nH]ncc23)cc(OC)c1CNCC(=O)N1CCN(CCN2CCC(c3ccc(NC4CCC(=O)NC4=O)cc3)CC2)CC1 ; COc1cc(-c2cn(C)c(=O)c3[nH]ncc23)cc(OC)c1CNCC(=O)NC1CCN(C(=O)CCCCC(=O)N2CCC(c3ccc(NC4CCC(=O)NC4=O)cc3)CC2)CC1 ; COc1cc(-c2cn(C)c(=O)c3[nH]ncc23)cc(OC)c1CNCC(=O)NCCCCCCCCN1CCC(c2ccc(NC3CCC(=O)NC3=O)cc2F)CC1]

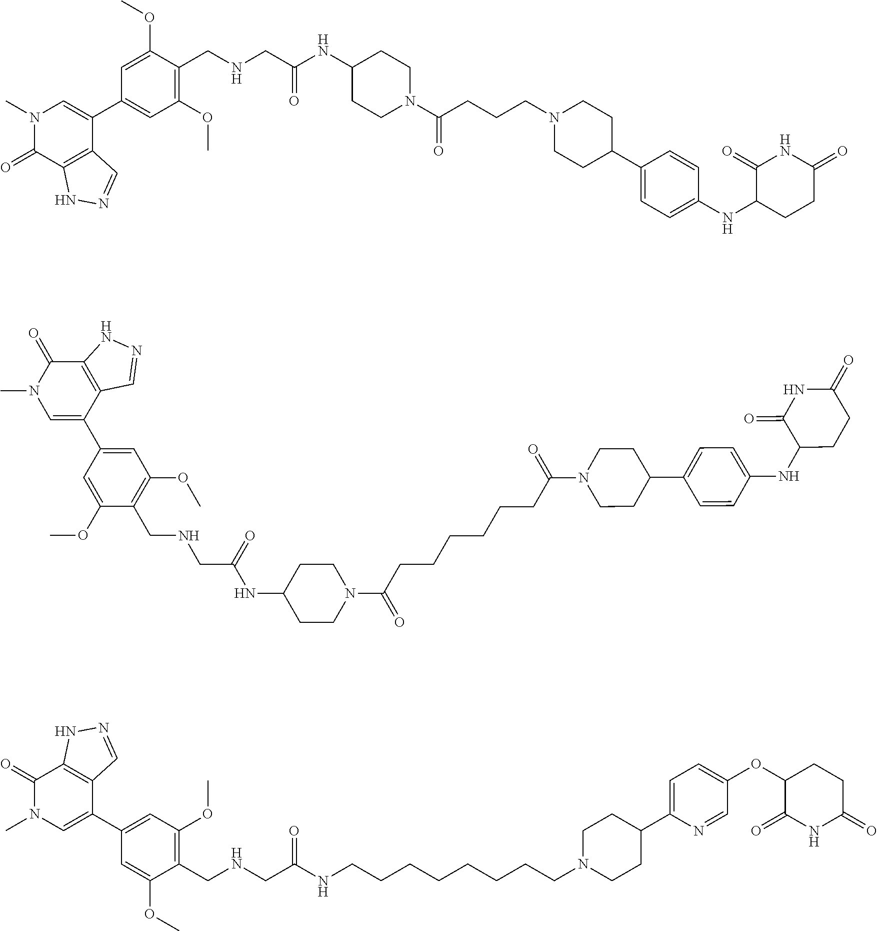 [SMILES: COc1cc(-c2cn(C)c(=O)c3[nH]ncc23)cc(OC)c1CNCC(=O)NC1CCN(C(=O)CCCN2CCC(c3ccc(NC4CCC(=O)NC4=O)cc3)CC2)CC1 ; COc1cc(-c2cn(C)c(=O)c3[nH]ncc23)cc(OC)c1CNCC(=O)NC1CCN(C(=O)CCCCCCC(=O)N2CCC(c3ccc(NC4CCC(=O)NC4=O)cc3)CC2)CC1 ; COc1cc(-c2cn(C)c(=O)c3[nH]ncc23)cc(OC)c1CNCC(=O)NCCCCCCCCN1CCC(c2ccc(OC3CCC(=O)NC3=O)cn2)CC1]

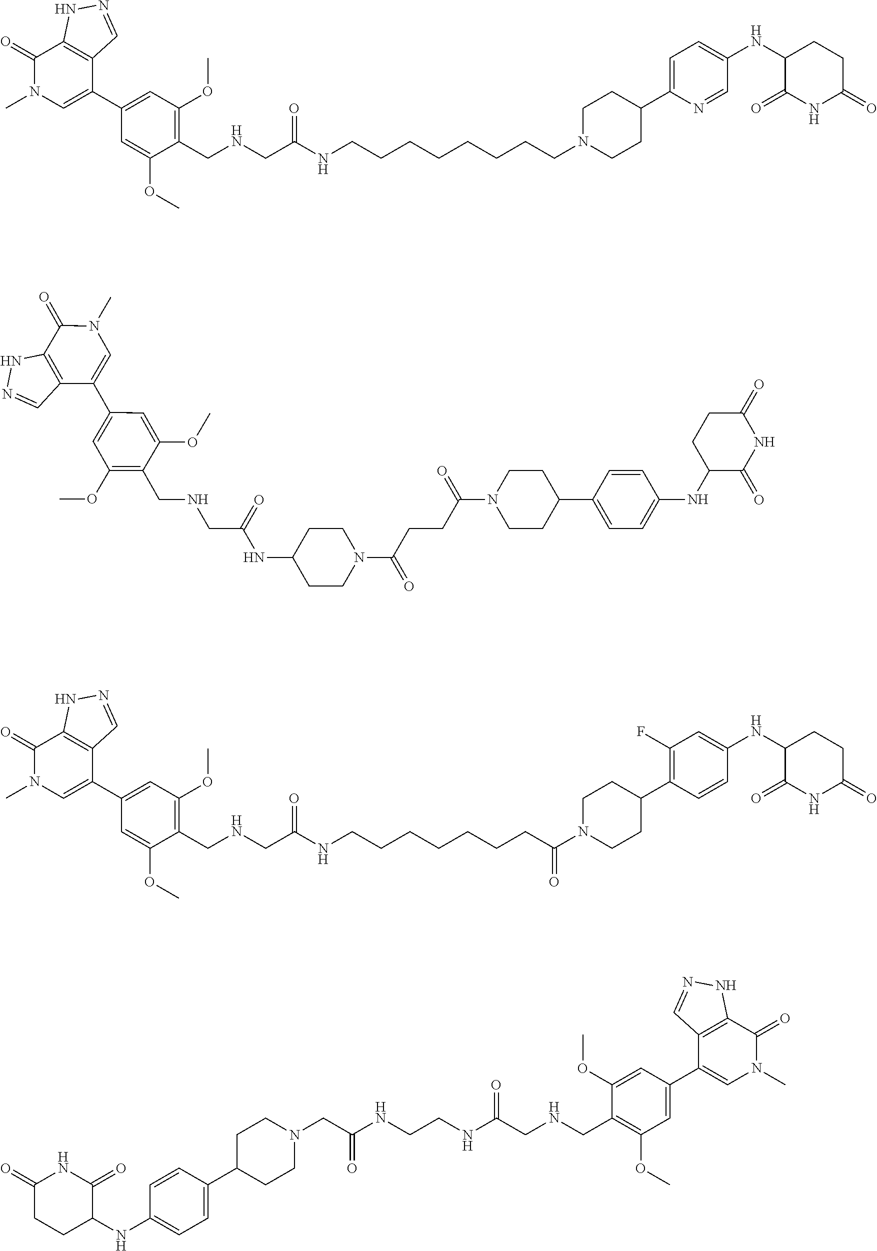 [SMILES: COc1cc(-c2cn(C)c(=O)c3[nH]ncc23)cc(OC)c1CNCC(=O)NCCCCCCCCN1CCC(c2ccc(NC3CCC(=O)NC3=O)cn2)CC1 ; COc1cc(-c2cn(C)c(=O)c3[nH]ncc23)cc(OC)c1CNCC(=O)NC1CCN(C(=O)CCC(=O)N2CCC(c3ccc(NC4CCC(=O)NC4=O)cc3)CC2)CC1 ; COc1cc(-c2cn(C)c(=O)c3[nH]ncc23)cc(OC)c1CNCC(=O)NCCCCCCCC(=O)N1CCC(c2ccc(NC3CCC(=O)NC3=O)cc2F)CC1 ; COc1cc(-c2cn(C)c(=O)c3[nH]ncc23)cc(OC)c1CNCC(=O)NCCNC(=O)CN1CCC(c2ccc(NC3CCC(=O)NC3=O)cc2)CC1]

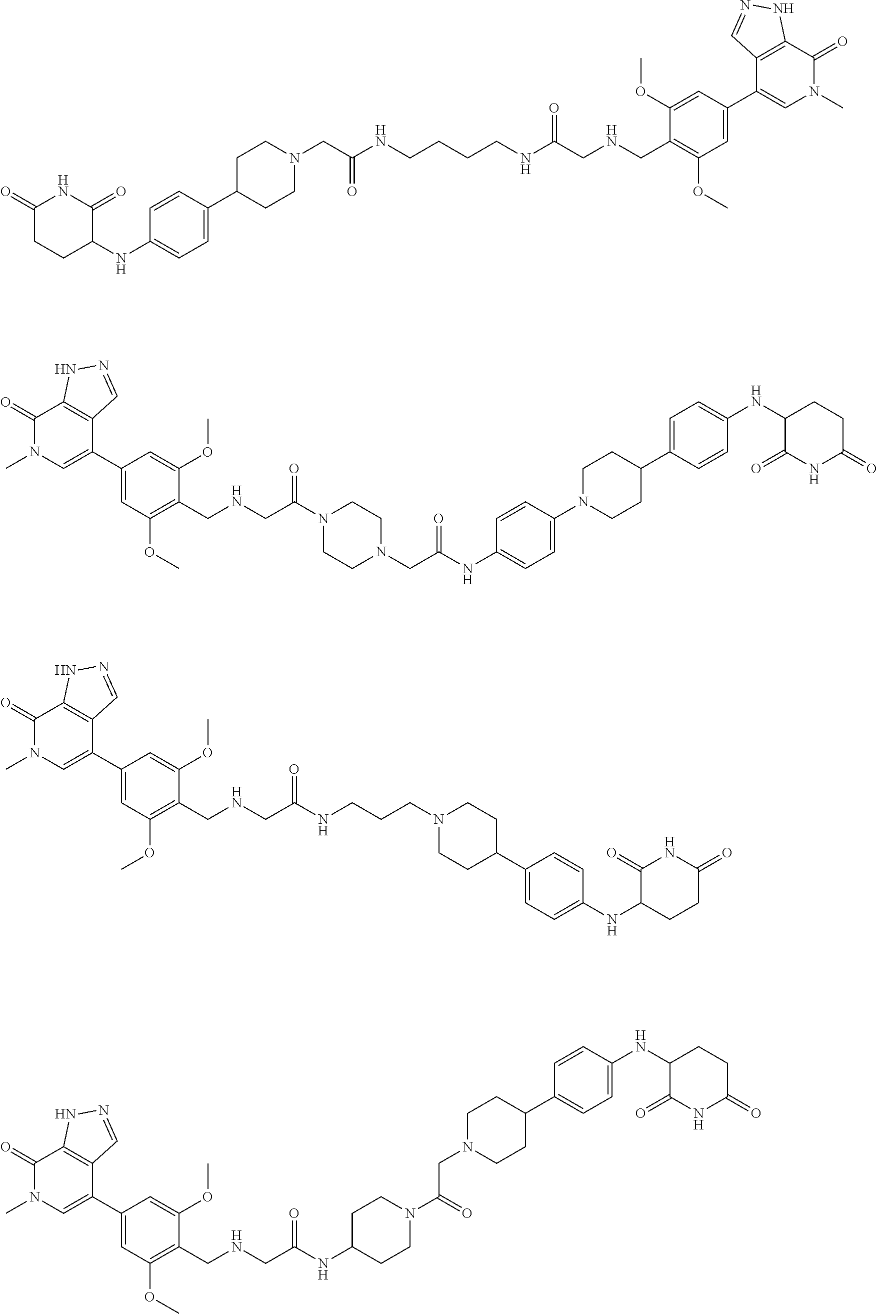 [SMILES: COc1cc(-c2cn(C)c(=O)c3[nH]ncc23)cc(OC)c1CNCC(=O)NCCCCNC(=O)CN1CCC(c2ccc(NC3CCC(=O)NC3=O)cc2)CC1 ; COc1cc(-c2cn(C)c(=O)c3[nH]ncc23)cc(OC)c1CNCC(=O)N1CCN(CC(=O)Nc2ccc(N3CCC(c4ccc(NC5CCC(=O)NC5=O)cc4)CC3)cc2)CC1 ; COc1cc(-c2cn(C)c(=O)c3[nH]ncc23)cc(OC)c1CNCC(=O)NCCCN1CCC(c2ccc(NC3CCC(=O)NC3=O)cc2)CC1 ; COc1cc(-c2cn(C)c(=O)c3[nH]ncc23)cc(OC)c1CNCC(=O)NC1CCN(C(=O)CN2CCC(c3ccc(NC4CCC(=O)NC4=O)cc3)CC2)CC1]

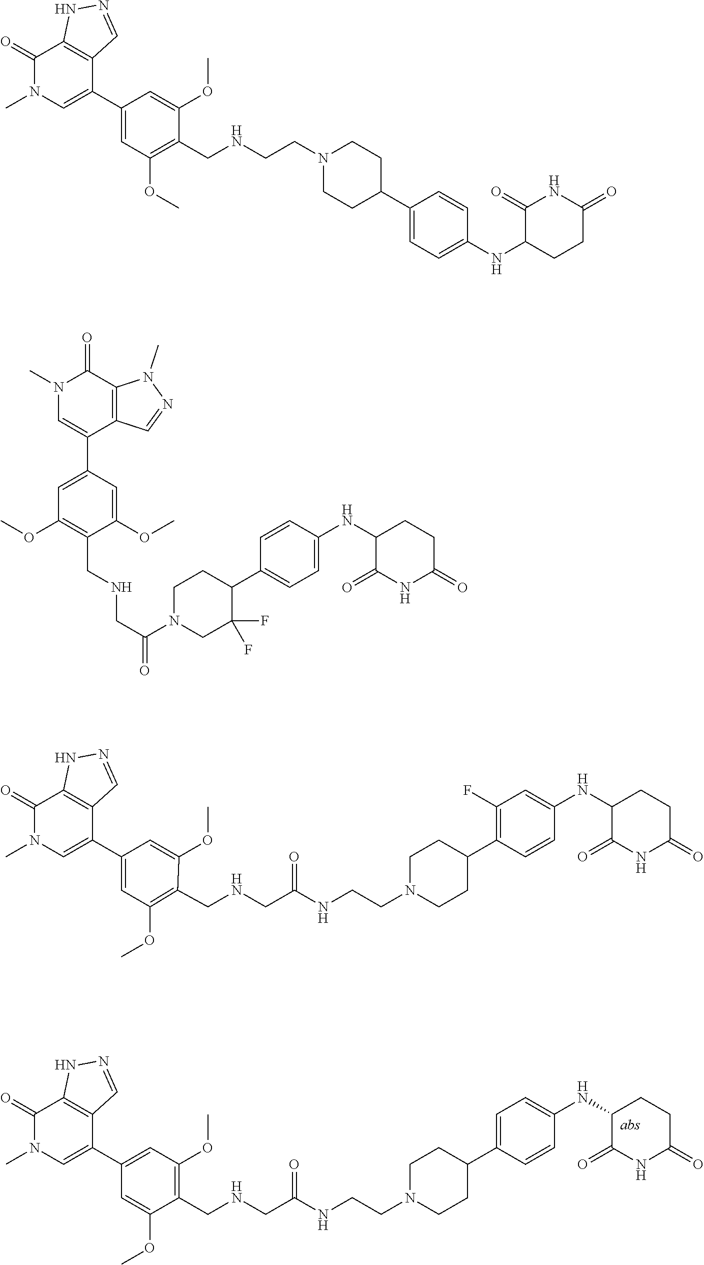 [SMILES: COc1cc(-c2cn(C)c(=O)c3[nH]ncc23)cc(OC)c1CNCCN1CCC(c2ccc(NC3CCC(=O)NC3=O)cc2)CC1 ; COc1cc(-c2cn(C)c(=O)c3c2cnn3C)cc(OC)c1CNCC(=O)N1CCC(c2ccc(NC3CCC(=O)NC3=O)cc2)C(F)(F)C1 ; COc1cc(-c2cn(C)c(=O)c3[nH]ncc23)cc(OC)c1CNCC(=O)NCCN1CCC(c2ccc(NC3CCC(=O)NC3=O)cc2F)CC1 ; COc1cc(-c2cn(C)c(=O)c3[nH]ncc23)cc(OC)c1CNCC(=O)NCCN1CCC(c2ccc(N[C@@H]3CCC(=O)NC3=O)cc2)CC1]

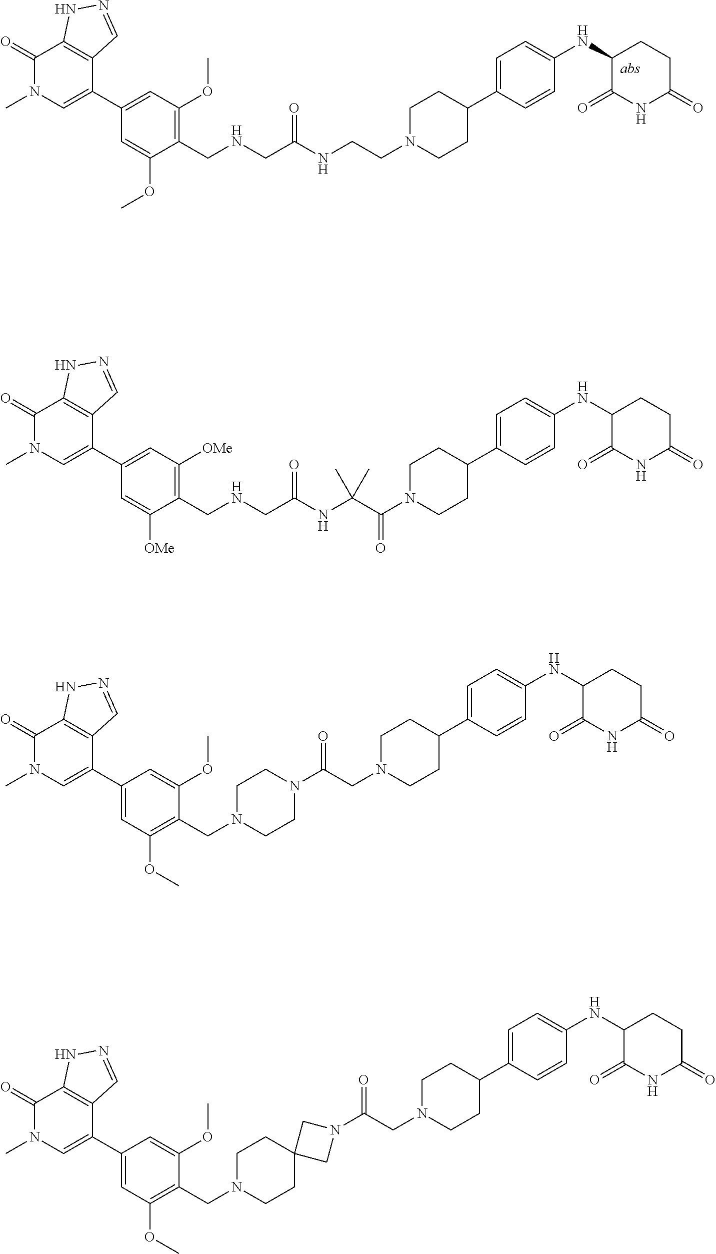 [SMILES: COc1cc(-c2cn(C)c(=O)c3[nH]ncc23)cc(OC)c1CNCC(=O)NCCN1CCC(c2ccc(N[C@H]3CCC(=O)NC3=O)cc2)CC1 ; COc1cc(-c2cn(C)c(=O)c3[nH]ncc23)cc(OC)c1CNCC(=O)NC(C)(C)C(=O)N1CCC(c2ccc(NC3CCC(=O)NC3=O)cc2)CC1 ; COc1cc(-c2cn(C)c(=O)c3[nH]ncc23)cc(OC)c1CN1CCN(C(=O)CN2CCC(c3ccc(NC4CCC(=O)NC4=O)cc3)CC2)CC1 ; COc1cc(-c2cn(C)c(=O)c3[nH]ncc23)cc(OC)c1CN1CCC2(CC1)CN(C(=O)CN1CCC(c3ccc(NC4CCC(=O)NC4=O)cc3)CC1)C2]

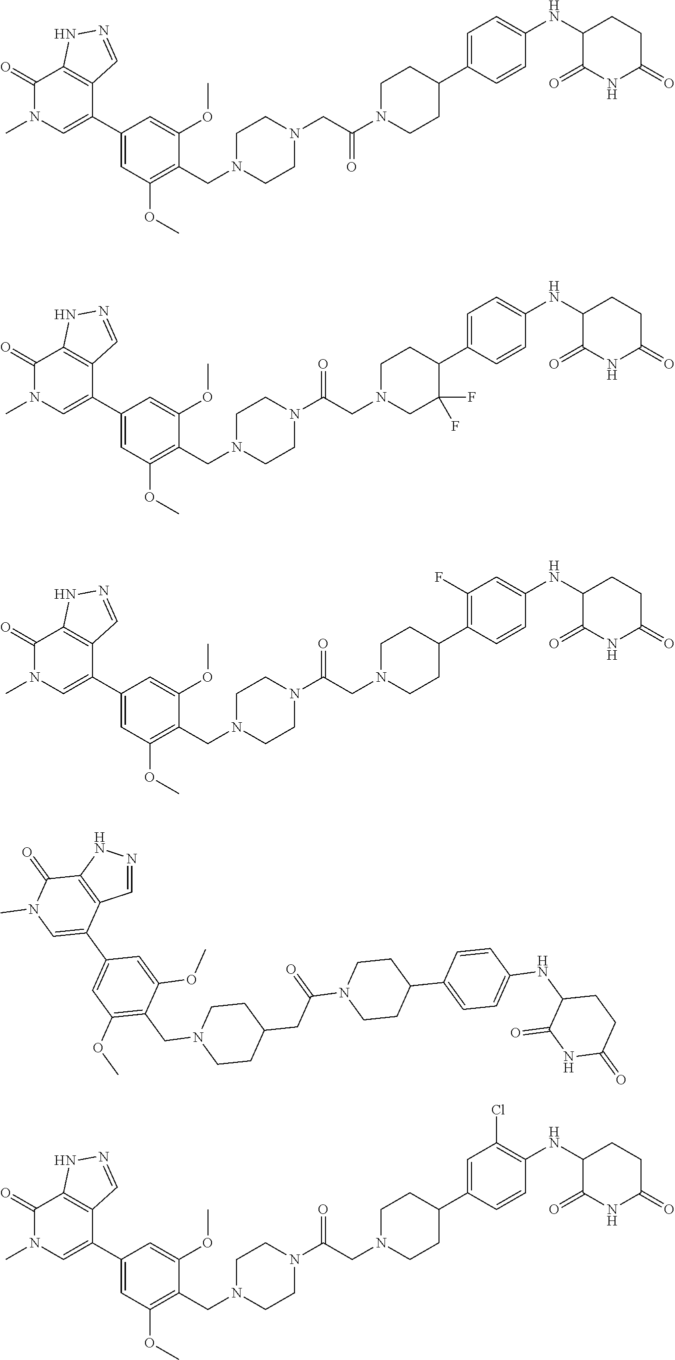 [SMILES: COc1cc(-c2cn(C)c(=O)c3[nH]ncc23)cc(OC)c1CN1CCN(CC(=O)N2CCC(c3ccc(NC4CCC(=O)NC4=O)cc3)CC2)CC1 ; COc1cc(-c2cn(C)c(=O)c3[nH]ncc23)cc(OC)c1CN1CCN(C(=O)CN2CCC(c3ccc(NC4CCC(=O)NC4=O)cc3)C(F)(F)C2)CC1 ; COc1cc(-c2cn(C)c(=O)c3[nH]ncc23)cc(OC)c1CN1CCN(C(=O)CN2CCC(c3ccc(NC4CCC(=O)NC4=O)cc3F)CC2)CC1 ; COc1cc(-c2cn(C)c(=O)c3[nH]ncc23)cc(OC)c1CN1CCC(CC(=O)N2CCC(c3ccc(NC4CCC(=O)NC4=O)cc3)CC2)CC1 ; COc1cc(-c2cn(C)c(=O)c3[nH]ncc23)cc(OC)c1CN1CCN(C(=O)CN2CCC(c3ccc(NC4CCC(=O)NC4=O)c(Cl)c3)CC2)CC1]

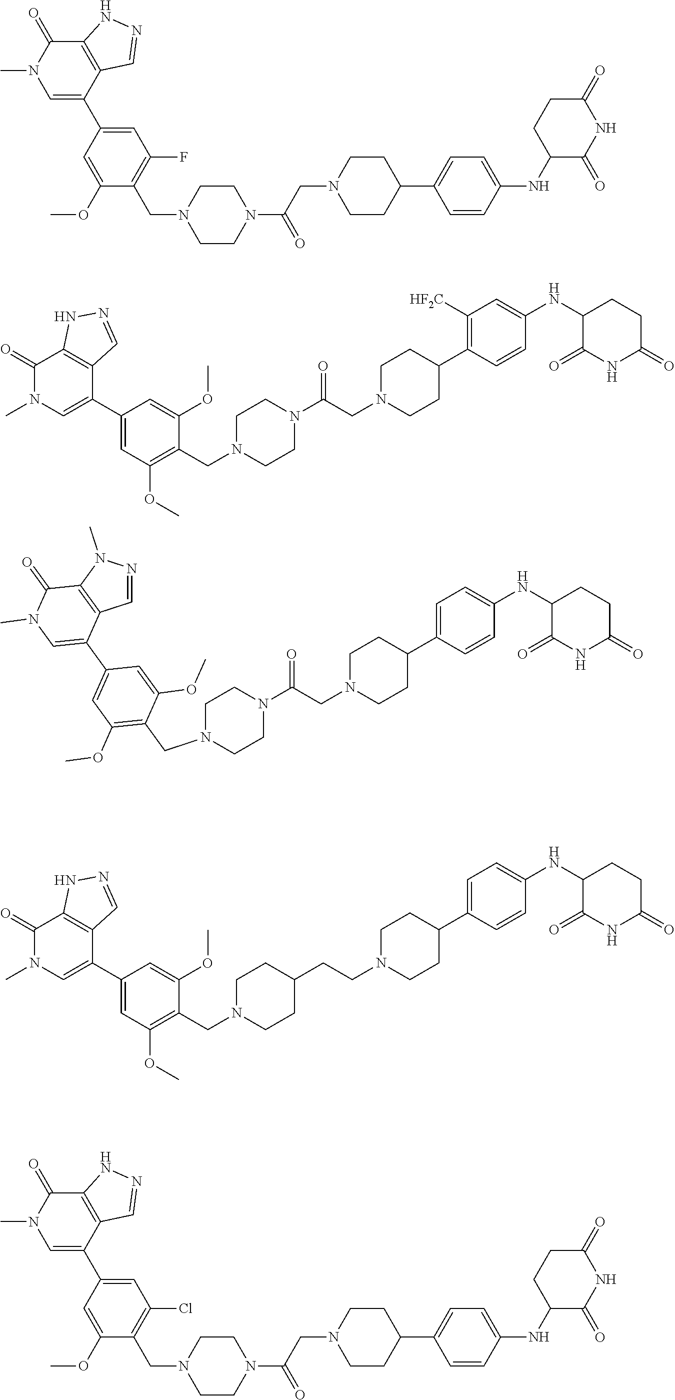

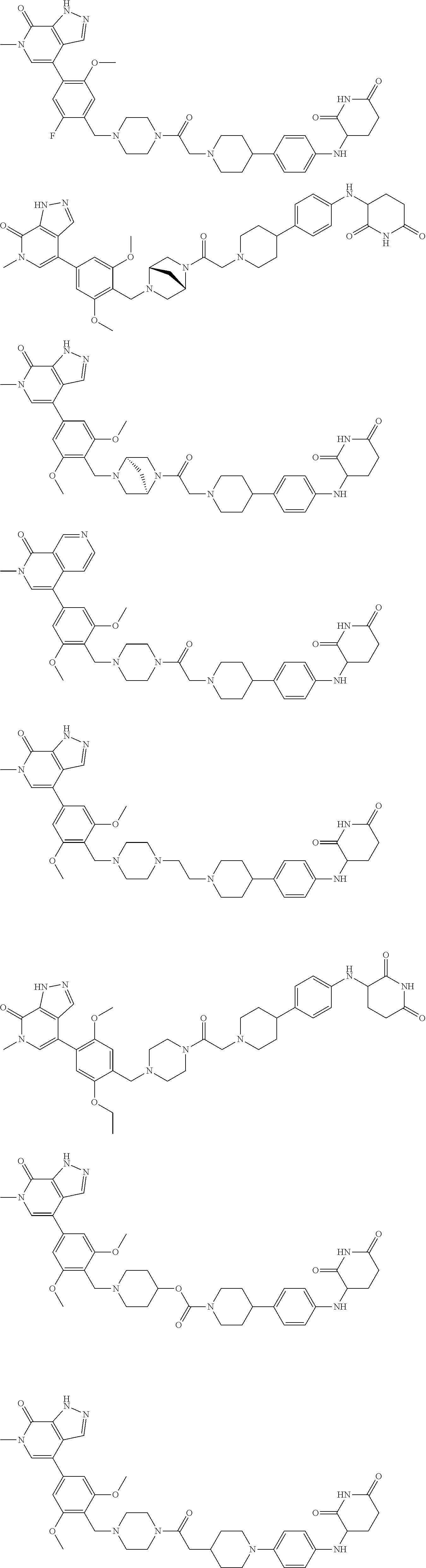

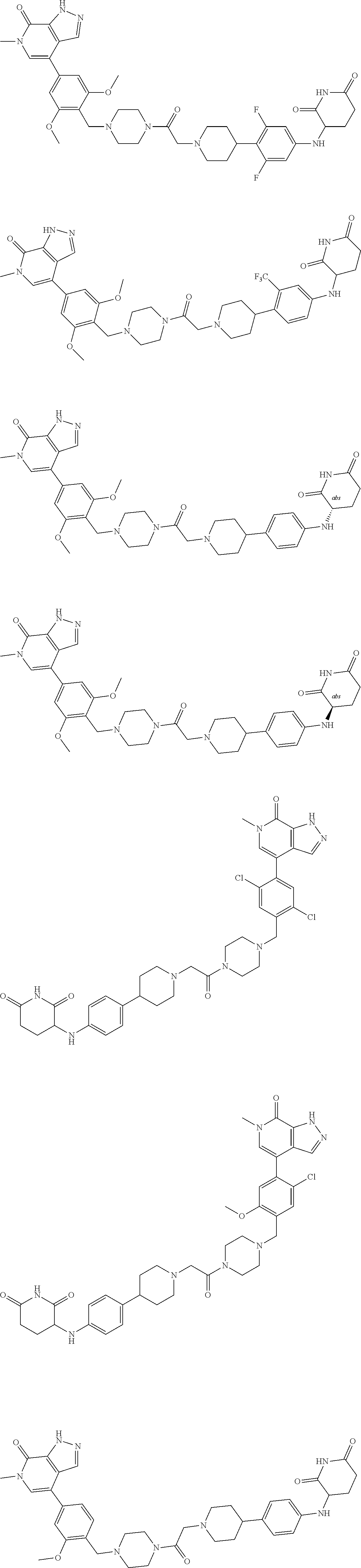

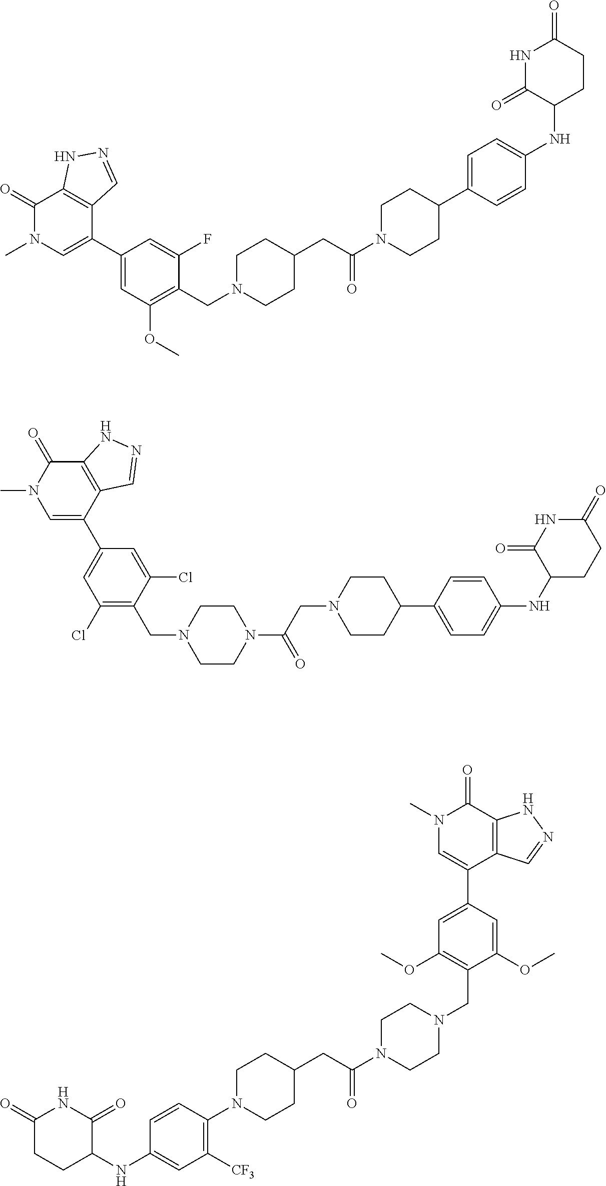 [SMILES: COc1cc(-c2cn(C)c(=O)c3[nH]ncc23)cc(F)c1CN1CCC(CC(=O)N2CCC(c3ccc(NC4CCC(=O)NC4=O)cc3)CC2)CC1 ; Cn1cc(-c2cc(Cl)c(CN3CCN(C(=O)CN4CCC(c5ccc(NC6CCC(=O)NC6=O)cc5)CC4)CC3)c(Cl)c2)c2cn[nH]c2c1=O ; COc1cc(-c2cn(C)c(=O)c3[nH]ncc23)cc(OC)c1CN1CCN(C(=O)CC2CCN(c3ccc(NC4CCC(=O)NC4=O)cc3C(F)(F)F)CC2)CC1]

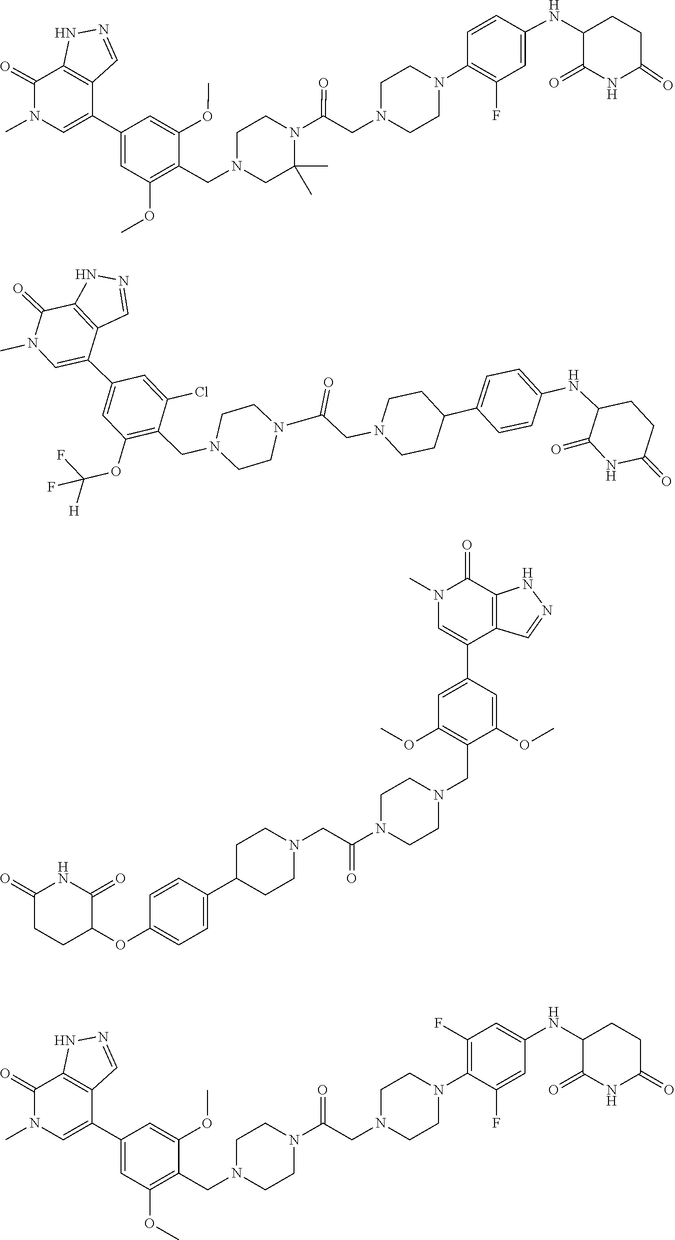

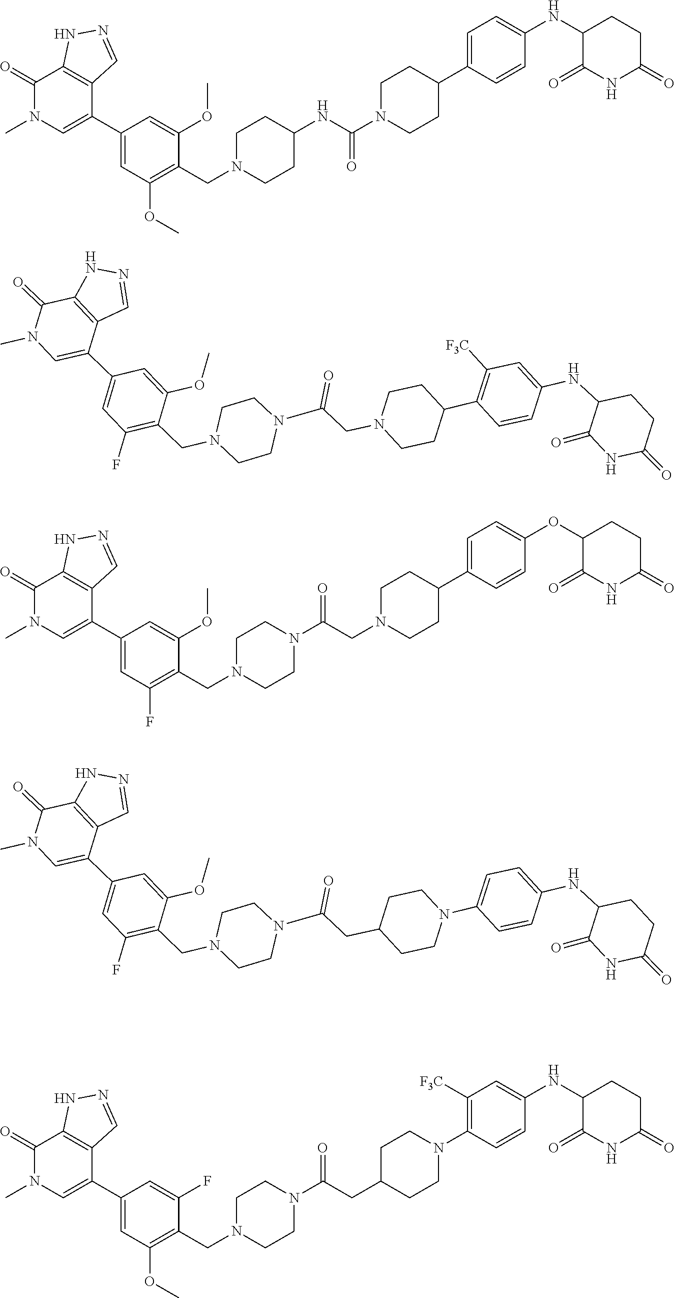 [SMILES: COc1cc(-c2cn(C)c(=O)c3[nH]ncc23)cc(OC)c1CN1CCC(NC(=O)N2CCC(c3ccc(NC4CCC(=O)NC4=O)cc3)CC2)CC1 ; COc1cc(-c2cn(C)c(=O)c3[nH]ncc23)cc(F)c1CN1CCN(C(=O)CN2CCC(c3ccc(NC4CCC(=O)NC4=O)cc3C(F)(F)F)CC2)CC1 ; COc1cc(-c2cn(C)c(=O)c3[nH]ncc23)cc(F)c1CN1CCN(C(=O)CN2CCC(c3ccc(OC4CCC(=O)NC4=O)cc3)CC2)CC1 ; COc1cc(-c2cn(C)c(=O)c3[nH]ncc23)cc(F)c1CN1CCN(C(=O)CC2CCN(c3ccc(NC4CCC(=O)NC4=O)cc3)CC2)CC1 ; COc1cc(-c2cn(C)c(=O)c3[nH]ncc23)cc(F)c1CN1CCN(C(=O)CC2CCN(c3ccc(NC4CCC(=O)NC4=O)cc3C(F)(F)F)CC2)CC1]

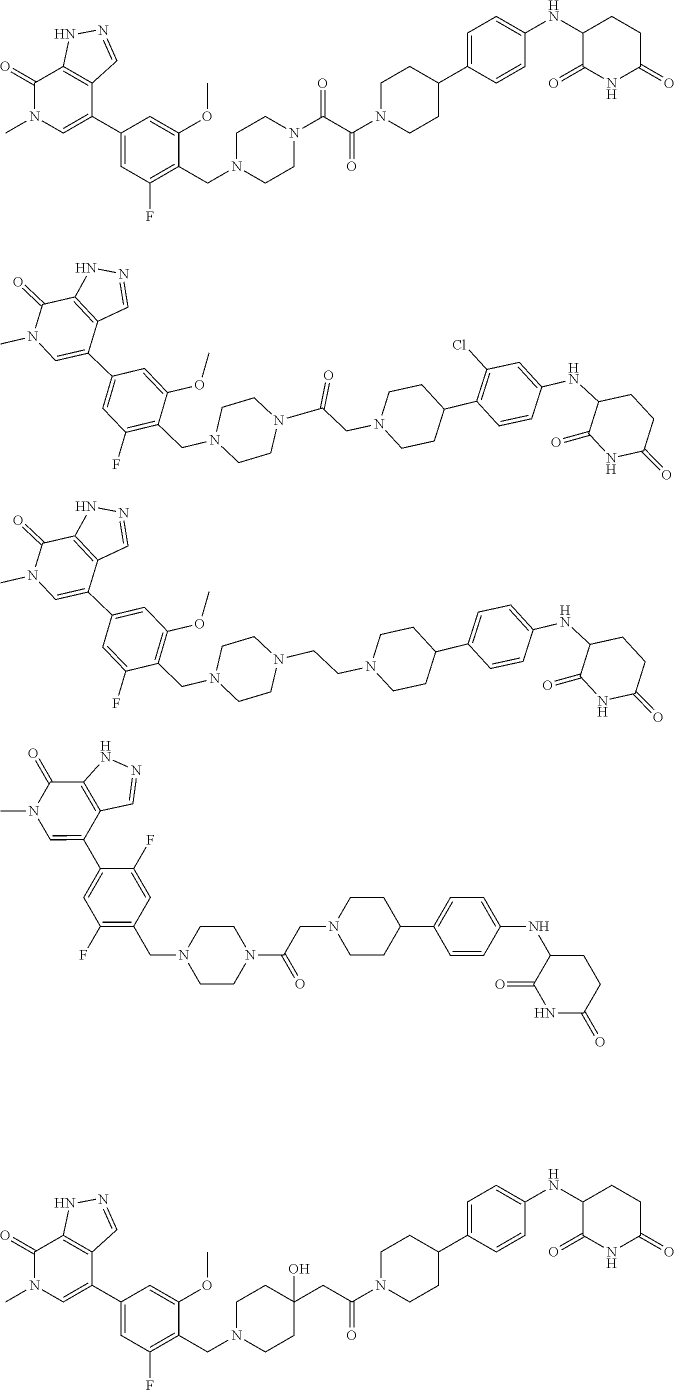 [SMILES: COc1cc(-c2cn(C)c(=O)c3[nH]ncc23)cc(F)c1CN1CCN(C(=O)C(=O)N2CCC(c3ccc(NC4CCC(=O)NC4=O)cc3)CC2)CC1 ; COc1cc(-c2cn(C)c(=O)c3[nH]ncc23)cc(F)c1CN1CCN(C(=O)CN2CCC(c3ccc(NC4CCC(=O)NC4=O)cc3Cl)CC2)CC1 ; COc1cc(-c2cn(C)c(=O)c3[nH]ncc23)cc(F)c1CN1CCN(CCN2CCC(c3ccc(NC4CCC(=O)NC4=O)cc3)CC2)CC1 ; Cn1cc(-c2cc(F)c(CN3CCN(C(=O)CN4CCC(c5ccc(NC6CCC(=O)NC6=O)cc5)CC4)CC3)cc2F)c2cn[nH]c2c1=O ; COc1cc(-c2cn(C)c(=O)c3[nH]ncc23)cc(F)c1CN1CCC(O)(CC(=O)N2CCC(c3ccc(NC4CCC(=O)NC4=O)cc3)CC2)CC1]

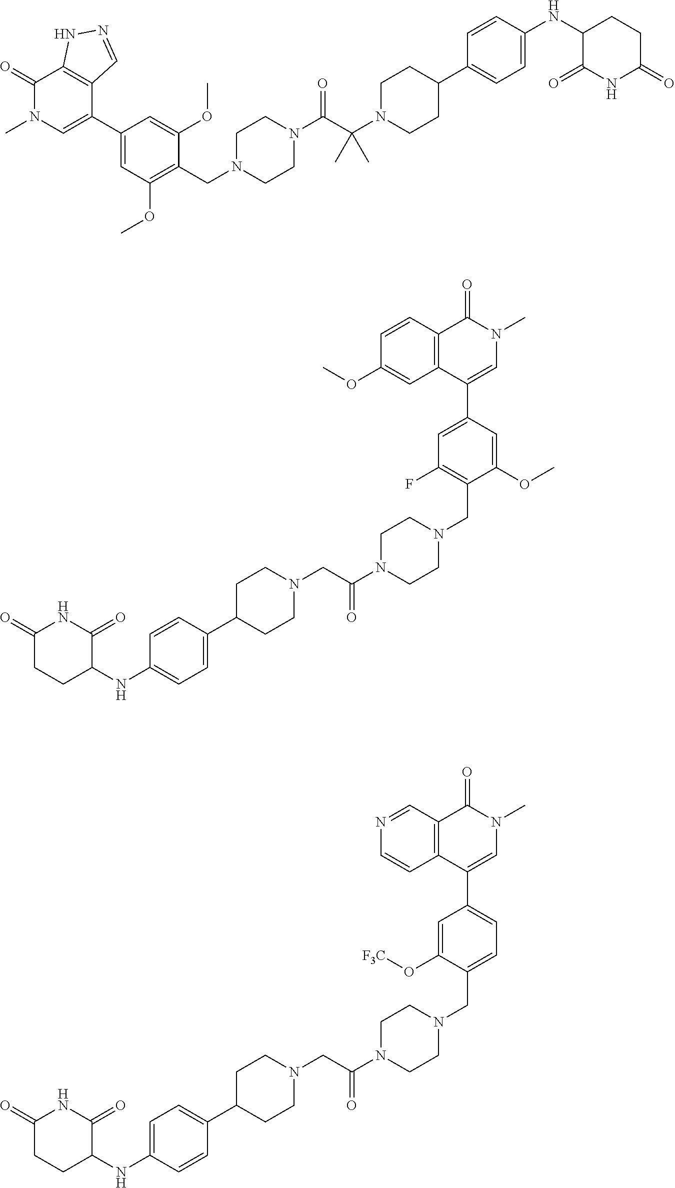 [SMILES: COc1cc(-c2cn(C)c(=O)c3[nH]ncc23)cc(OC)c1CN1CCN(C(=O)C(C)(C)N2CCC(c3ccc(NC4CCC(=O)NC4=O)cc3)CC2)CC1 ; COc1ccc2c(=O)n(C)cc(-c3cc(F)c(CN4CCN(C(=O)CN5CCC(c6ccc(NC7CCC(=O)NC7=O)cc6)CC5)CC4)c(OC)c3)c2c1 ; Cn1cc(-c2ccc(CN3CCN(C(=O)CN4CCC(c5ccc(NC6CCC(=O)NC6=O)cc5)CC4)CC3)c(OC(F)(F)F)c2)c2ccncc2c1=O]

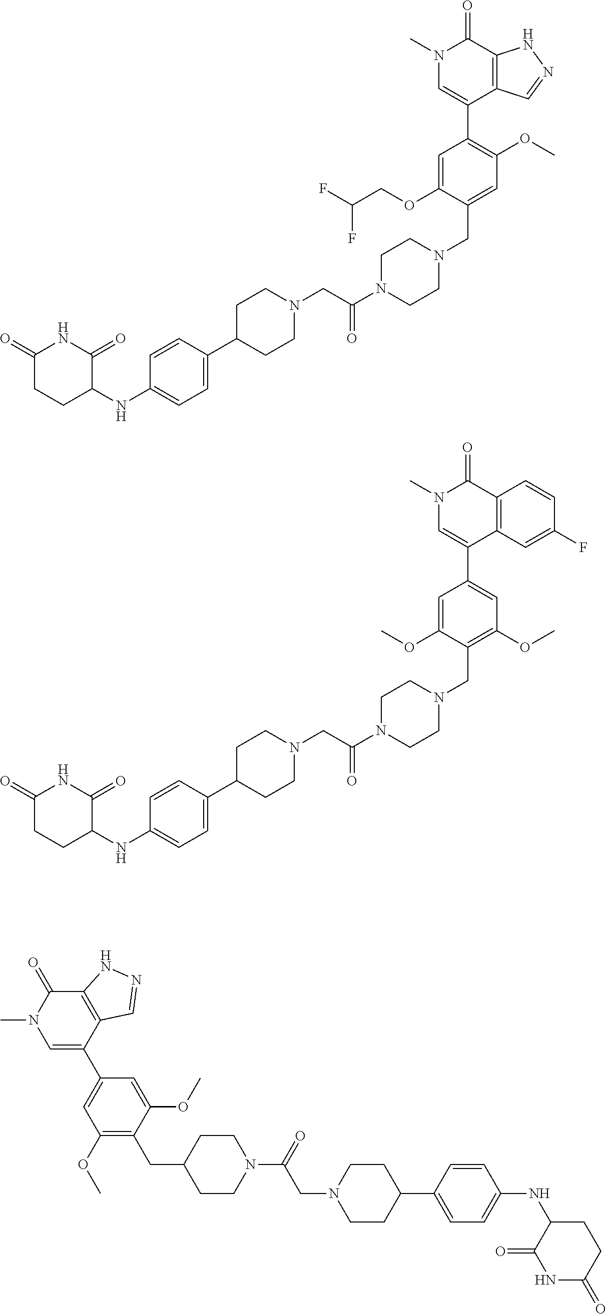

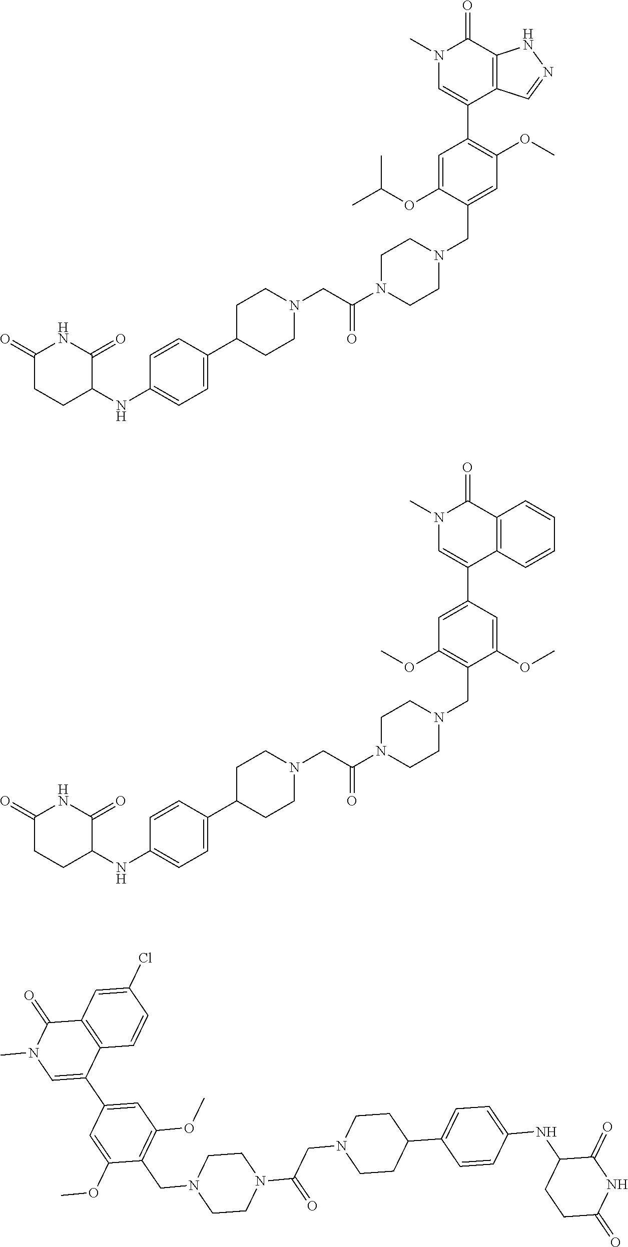

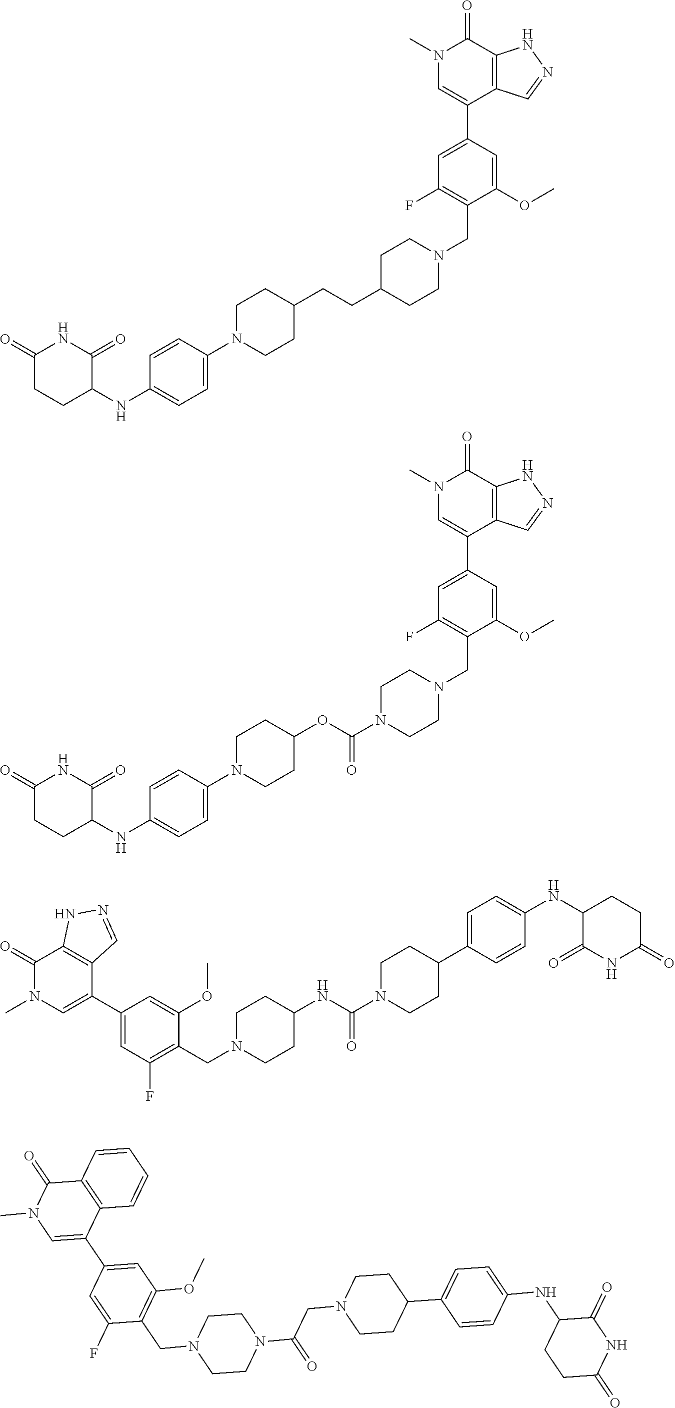

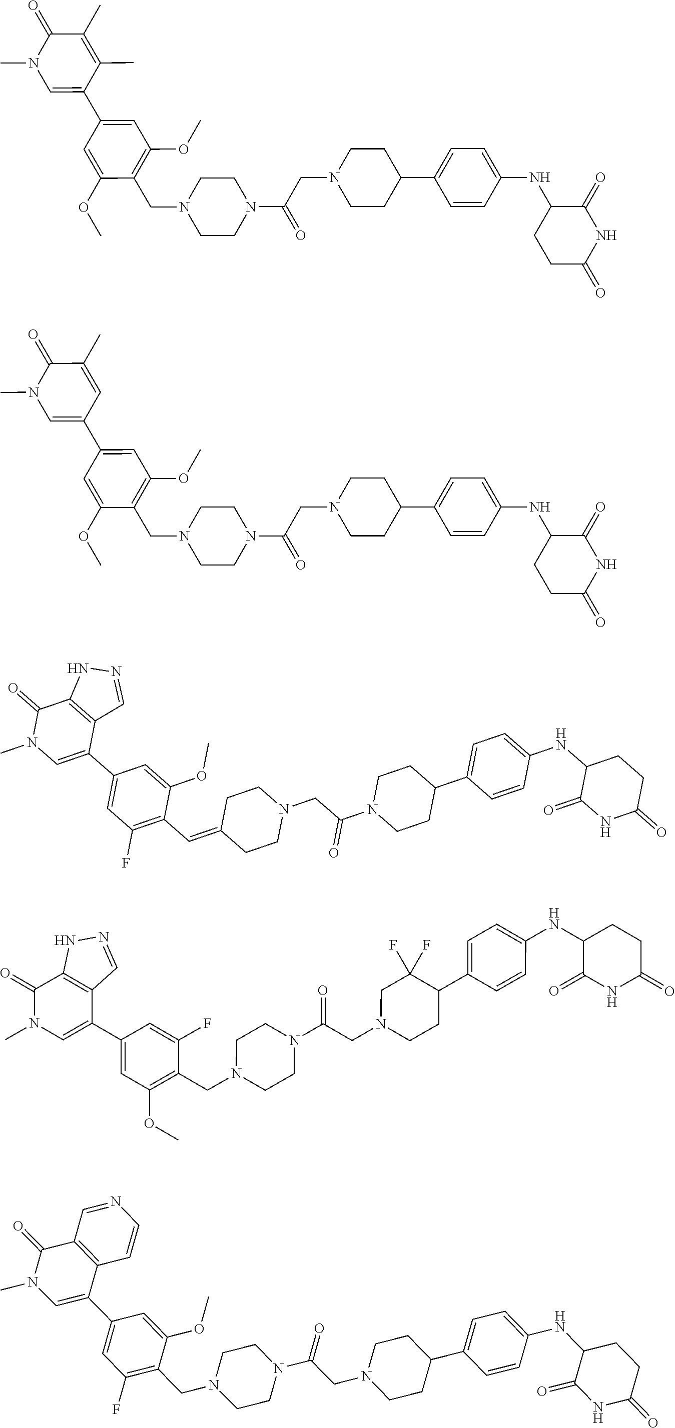 [SMILES: COc1cc(-c2cn(C)c(=O)c(C)c2C)cc(OC)c1CN1CCN(C(=O)CN2CCC(c3ccc(NC4CCC(=O)NC4=O)cc3)CC2)CC1 ; COc1cc(-c2cc(C)c(=O)n(C)c2)cc(OC)c1CN1CCN(C(=O)CN2CCC(c3ccc(NC4CCC(=O)NC4=O)cc3)CC2)CC1 ; COc1cc(-c2cn(C)c(=O)c3[nH]ncc23)cc(F)c1C=C1CCN(CC(=O)N2CCC(c3ccc(NC4CCC(=O)NC4=O)cc3)CC2)CC1 ; COc1cc(-c2cn(C)c(=O)c3[nH]ncc23)cc(F)c1CN1CCN(C(=O)CN2CCC(c3ccc(NC4CCC(=O)NC4=O)cc3)C(F)(F)C2)CC1 ; COc1cc(-c2cn(C)c(=O)c3cnccc23)cc(F)c1CN1CCN(C(=O)CN2CCC(c3ccc(NC4CCC(=O)NC4=O)cc3)CC2)CC1]

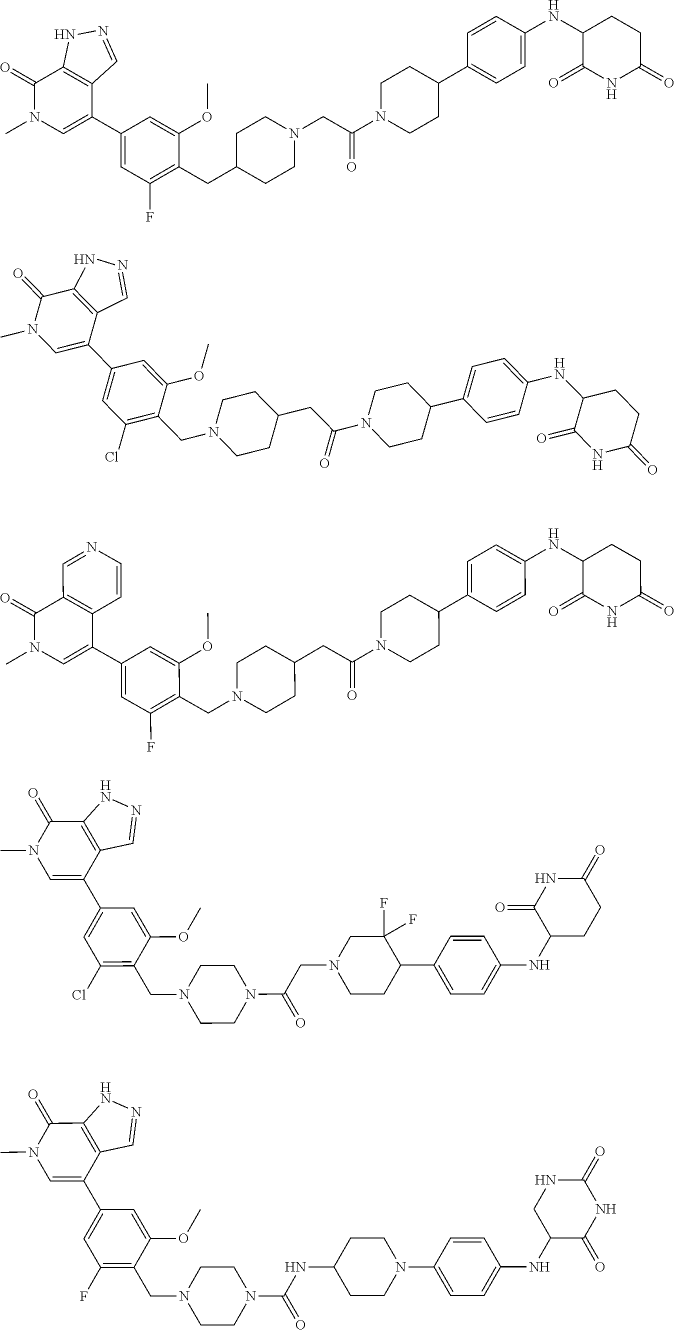 [SMILES: COc1cc(-c2cn(C)c(=O)c3[nH]ncc23)cc(F)c1CC1CCN(CC(=O)N2CCC(c3ccc(NC4CCC(=O)NC4=O)cc3)CC2)CC1 ; COc1cc(-c2cn(C)c(=O)c3[nH]ncc23)cc(Cl)c1CN1CCC(CC(=O)N2CCC(c3ccc(NC4CCC(=O)NC4=O)cc3)CC2)CC1 ; COc1cc(-c2cn(C)c(=O)c3cnccc23)cc(F)c1CN1CCC(CC(=O)N2CCC(c3ccc(NC4CCC(=O)NC4=O)cc3)CC2)CC1 ; COc1cc(-c2cn(C)c(=O)c3[nH]ncc23)cc(Cl)c1CN1CCN(C(=O)CN2CCC(c3ccc(NC4CCC(=O)NC4=O)cc3)C(F)(F)C2)CC1 ; COc1cc(-c2cn(C)c(=O)c3[nH]ncc23)cc(F)c1CN1CCN(C(=O)NC2CCN(c3ccc(NC4CNC(=O)NC4=O)cc3)CC2)CC1]

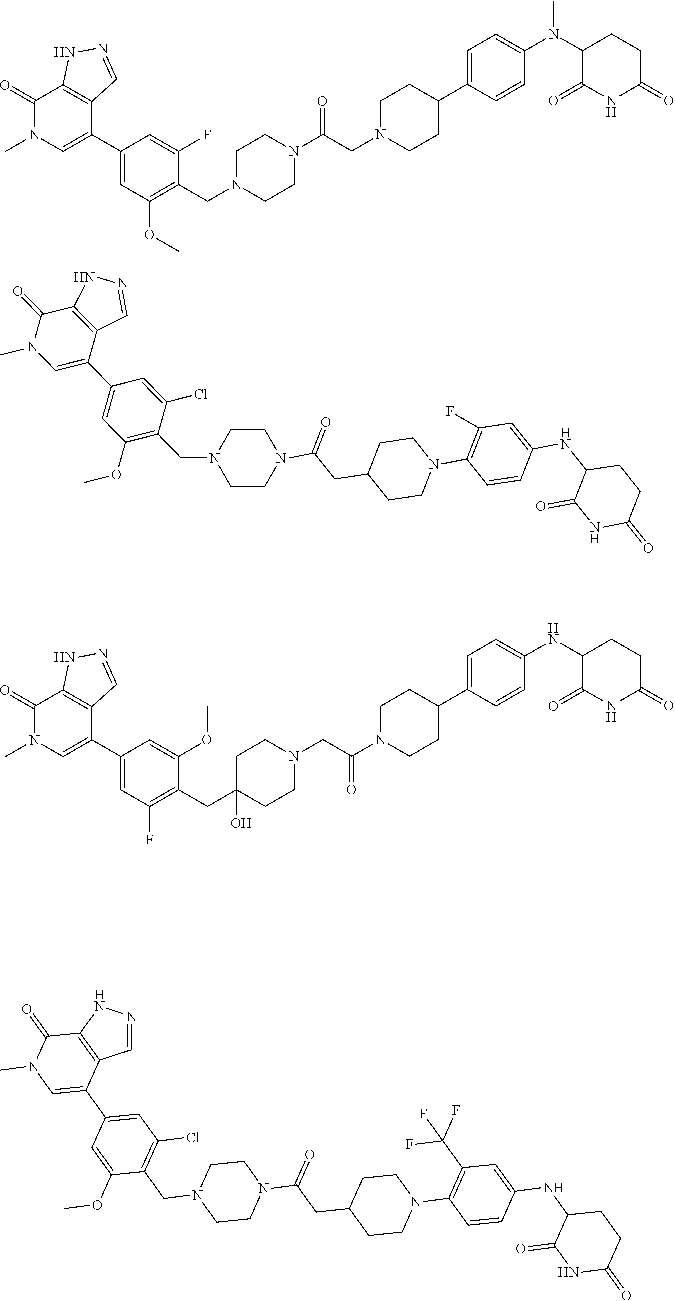 [SMILES: COc1cc(-c2cn(C)c(=O)c3[nH]ncc23)cc(F)c1CN1CCN(C(=O)CN2CCC(c3ccc(N(C)C4CCC(=O)NC4=O)cc3)CC2)CC1 ; COc1cc(-c2cn(C)c(=O)c3[nH]ncc23)cc(Cl)c1CN1CCN(C(=O)CC2CCN(c3ccc(NC4CCC(=O)NC4=O)cc3F)CC2)CC1 ; COc1cc(-c2cn(C)c(=O)c3[nH]ncc23)cc(F)c1CC1(O)CCN(CC(=O)N2CCC(c3ccc(NC4CCC(=O)NC4=O)cc3)CC2)CC1 ; COc1cc(-c2cn(C)c(=O)c3[nH]ncc23)cc(Cl)c1CN1CCN(C(=O)CC2CCN(c3ccc(NC4CCC(=O)NC4=O)cc3C(F)(F)F)CC2)CC1]

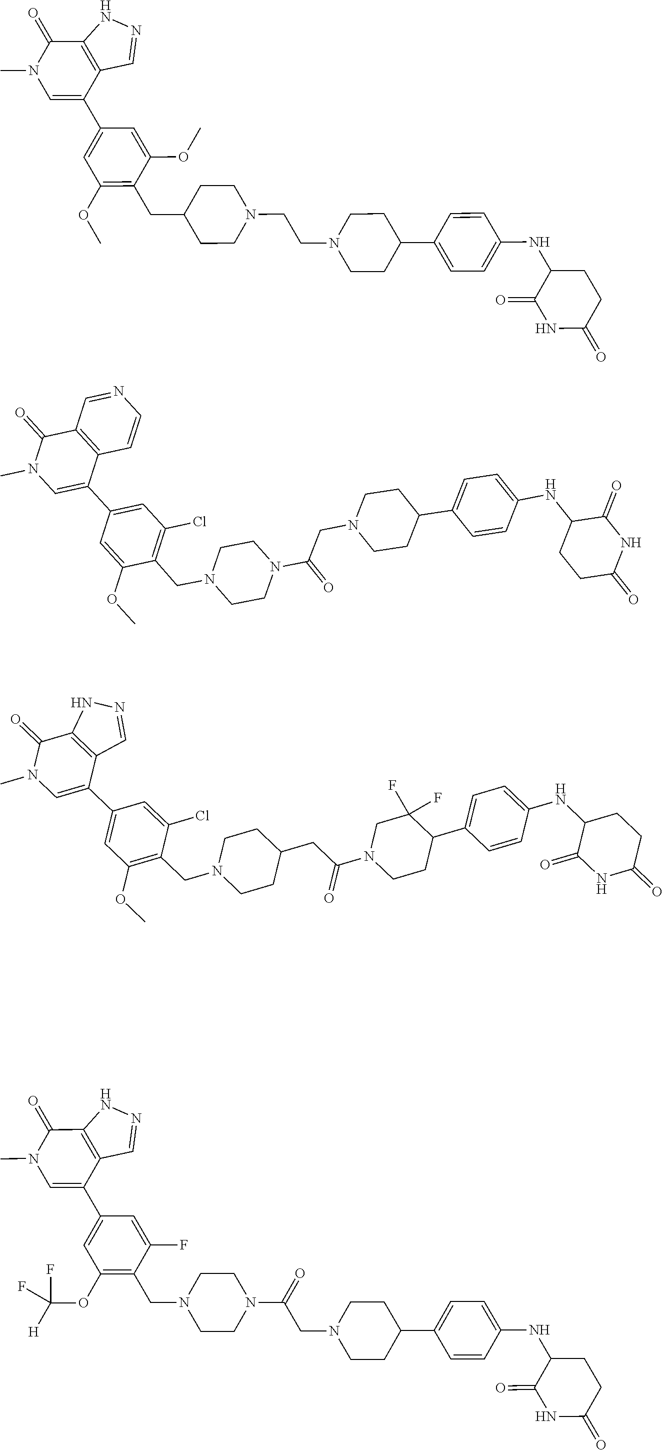 [SMILES: COc1cc(-c2cn(C)c(=O)c3[nH]ncc23)cc(OC)c1CC1CCN(CCN2CCC(c3ccc(NC4CCC(=O)NC4=O)cc3)CC2)CC1 ; COc1cc(-c2cn(C)c(=O)c3cnccc23)cc(Cl)c1CN1CCN(C(=O)CN2CCC(c3ccc(NC4CCC(=O)NC4=O)cc3)CC2)CC1 ; COc1cc(-c2cn(C)c(=O)c3[nH]ncc23)cc(Cl)c1CN1CCC(CC(=O)N2CCC(c3ccc(NC4CCC(=O)NC4=O)cc3)C(F)(F)C2)CC1 ; Cn1cc(-c2cc(F)c(CN3CCN(C(=O)CN4CCC(c5ccc(NC6CCC(=O)NC6=O)cc5)CC4)CC3)c(OC(F)F)c2)c2cn[nH]c2c1=O]

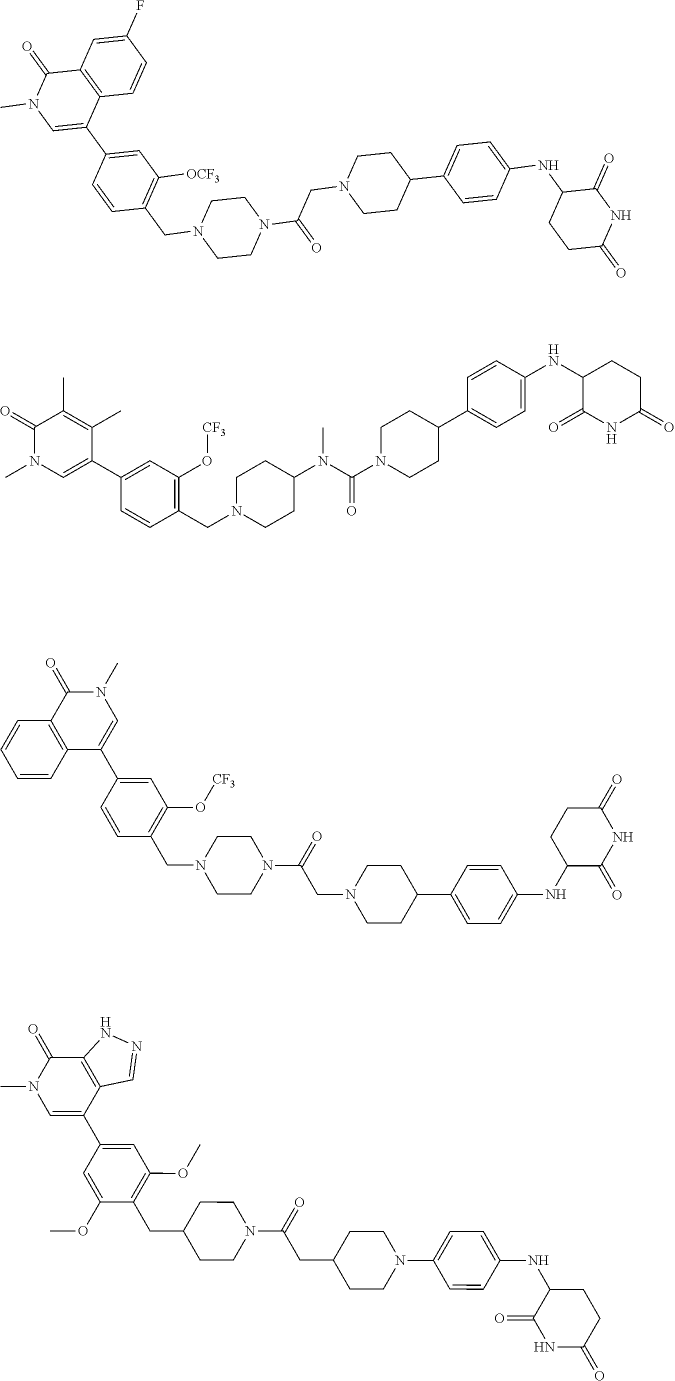 [SMILES: Cn1cc(-c2ccc(CN3CCN(C(=O)CN4CCC(c5ccc(NC6CCC(=O)NC6=O)cc5)CC4)CC3)c(OC(F)(F)F)c2)c2ccc(F)cc2c1=O ; Cc1c(-c2ccc(CN3CCC(N(C)C(=O)N4CCC(c5ccc(NC6CCC(=O)NC6=O)cc5)CC4)CC3)c(OC(F)(F)F)c2)cn(C)c(=O)c1C ; Cn1cc(-c2ccc(CN3CCN(C(=O)CN4CCC(c5ccc(NC6CCC(=O)NC6=O)cc5)CC4)CC3)c(OC(F)(F)F)c2)c2ccccc2c1=O ; COc1cc(-c2cn(C)c(=O)c3[nH]ncc23)cc(OC)c1CC1CCN(C(=O)CC2CCN(c3ccc(NC4CCC(=O)NC4=O)cc3)CC2)CC1]

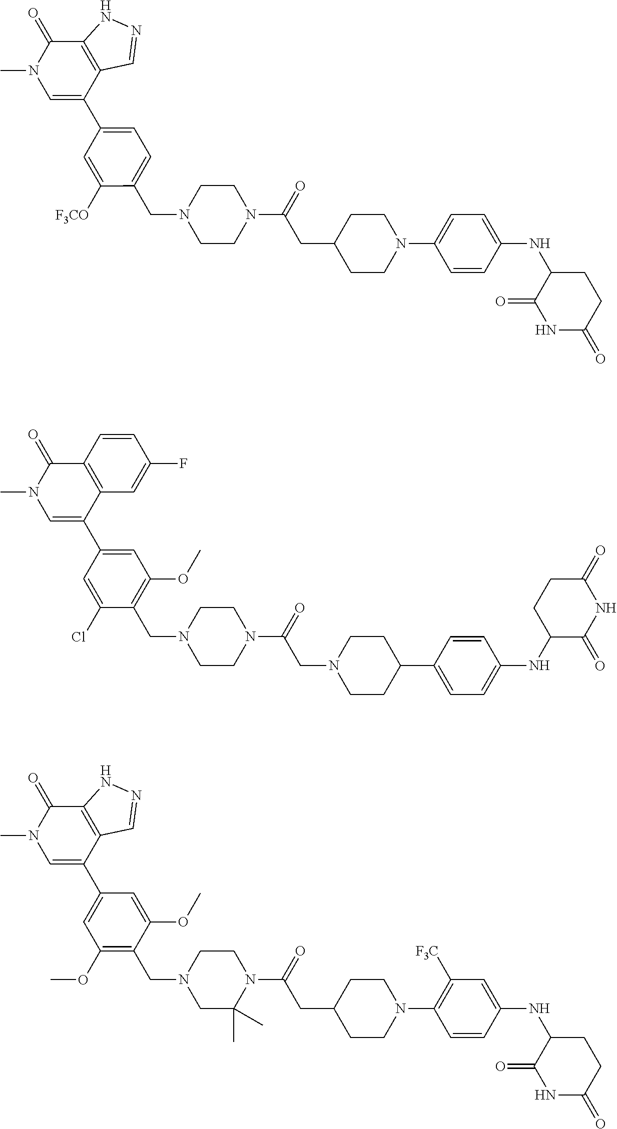 [SMILES: Cn1cc(-c2ccc(CN3CCN(C(=O)CC4CCN(c5ccc(NC6CCC(=O)NC6=O)cc5)CC4)CC3)c(OC(F)(F)F)c2)c2cn[nH]c2c1=O ; COc1cc(-c2cn(C)c(=O)c3ccc(F)cc23)cc(Cl)c1CN1CCN(C(=O)CN2CCC(c3ccc(NC4CCC(=O)NC4=O)cc3)CC2)CC1 ; COc1cc(-c2cn(C)c(=O)c3[nH]ncc23)cc(OC)c1CN1CCN(C(=O)CC2CCN(c3ccc(NC4CCC(=O)NC4=O)cc3C(F)(F)F)CC2)C(C)(C)C1]

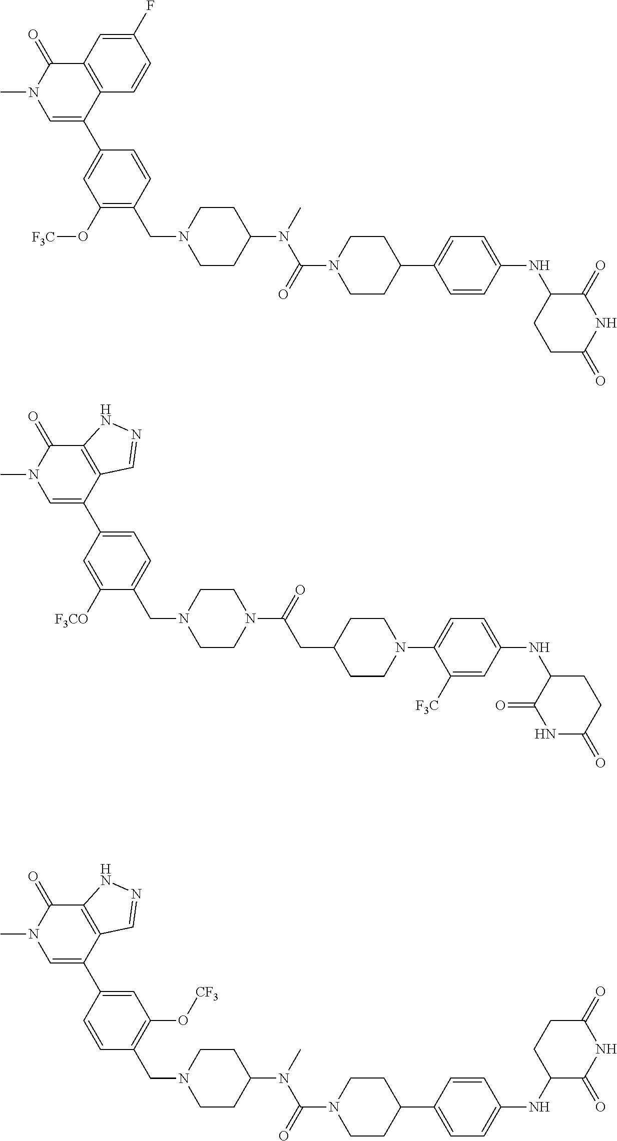 [SMILES: CN(C(=O)N1CCC(c2ccc(NC3CCC(=O)NC3=O)cc2)CC1)C1CCN(Cc2ccc(-c3cn(C)c(=O)c4cc(F)ccc34)cc2OC(F)(F)F)CC1 ; Cn1cc(-c2ccc(CN3CCN(C(=O)CC4CCN(c5ccc(NC6CCC(=O)NC6=O)cc5C(F)(F)F)CC4)CC3)c(OC(F)(F)F)c2)c2cn[nH]c2c1=O ; CN(C(=O)N1CCC(c2ccc(NC3CCC(=O)NC3=O)cc2)CC1)C1CCN(Cc2ccc(-c3cn(C)c(=O)c4[nH]ncc34)cc2OC(F)(F)F)CC1]

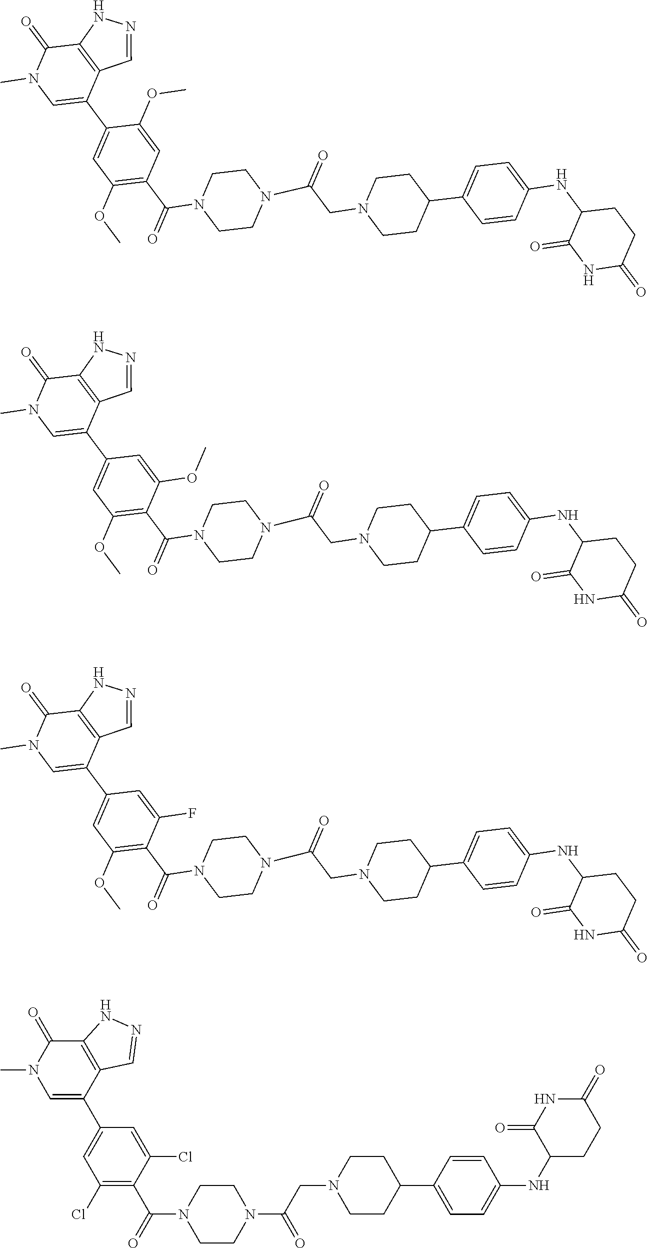 [SMILES: COc1cc(-c2cn(C)c(=O)c3[nH]ncc23)c(OC)cc1C(=O)N1CCN(C(=O)CN2CCC(c3ccc(NC4CCC(=O)NC4=O)cc3)CC2)CC1 ; COc1cc(-c2cn(C)c(=O)c3[nH]ncc23)cc(OC)c1C(=O)N1CCN(C(=O)CN2CCC(c3ccc(NC4CCC(=O)NC4=O)cc3)CC2)CC1 ; COc1cc(-c2cn(C)c(=O)c3[nH]ncc23)cc(F)c1C(=O)N1CCN(C(=O)CN2CCC(c3ccc(NC4CCC(=O)NC4=O)cc3)CC2)CC1 ; Cn1cc(-c2cc(Cl)c(C(=O)N3CCN(C(=O)CN4CCC(c5ccc(NC6CCC(=O)NC6=O)cc5)CC4)CC3)c(Cl)c2)c2cn[nH]c2c1=O]

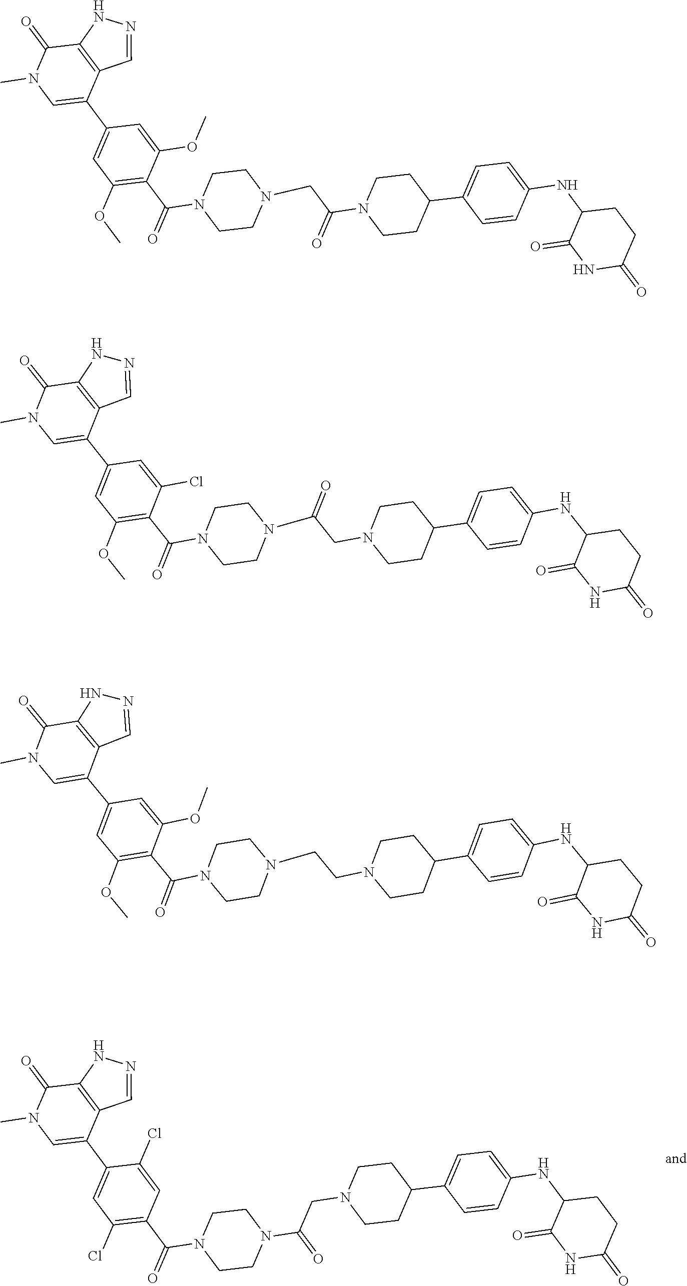 [SMILES: COc1cc(-c2cn(C)c(=O)c3[nH]ncc23)cc(OC)c1C(=O)N1CCN(CC(=O)N2CCC(c3ccc(NC4CCC(=O)NC4=O)cc3)CC2)CC1 ; COc1cc(-c2cn(C)c(=O)c3[nH]ncc23)cc(Cl)c1C(=O)N1CCN(C(=O)CN2CCC(c3ccc(NC4CCC(=O)NC4=O)cc3)CC2)CC1 ; COc1cc(-c2cn(C)c(=O)c3[nH]ncc23)cc(OC)c1C(=O)N1CCN(CCN2CCC(c3ccc(NC4CCC(=O)NC4=O)cc3)CC2)CC1 ; Cn1cc(-c2cc(Cl)c(C(=O)N3CCN(C(=O)CN4CCC(c5ccc(NC6CCC(=O)NC6=O)cc5)CC4)CC3)cc2Cl)c2cn[nH]c2c1=O]

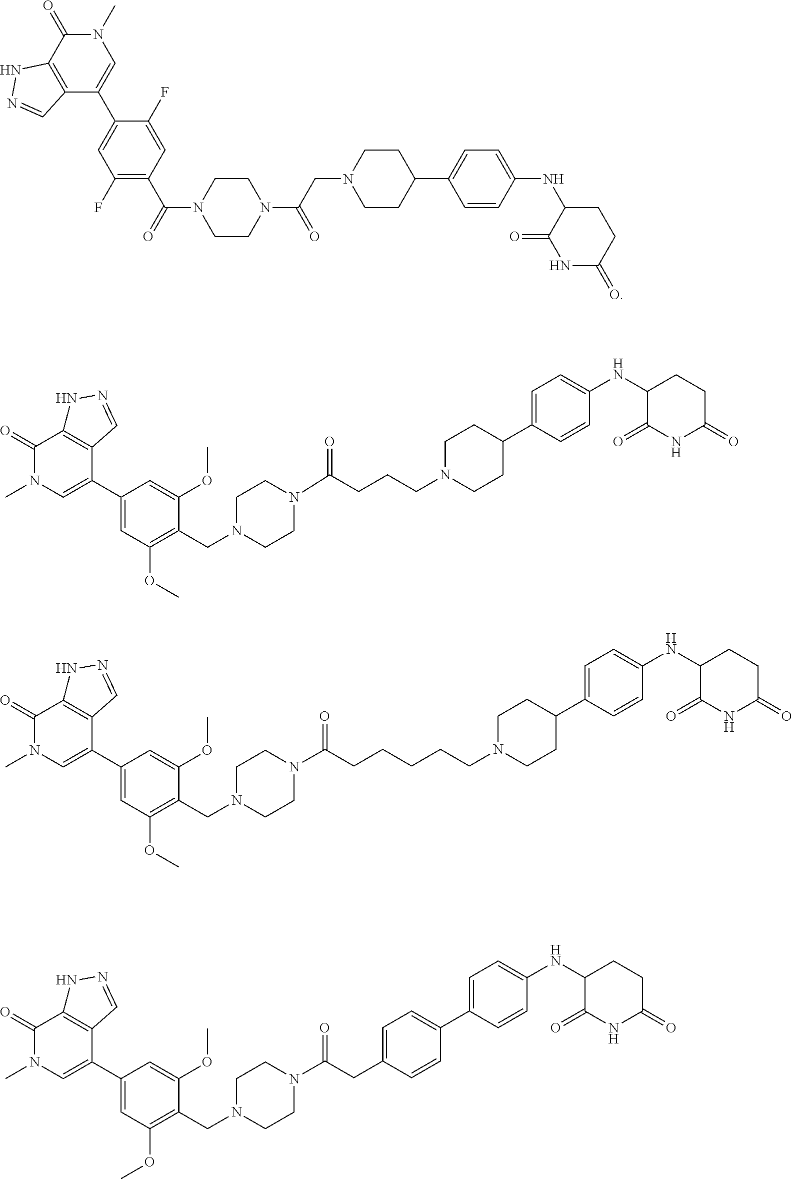 [SMILES: Cn1cc(-c2cc(F)c(C(=O)N3CCN(C(=O)CN4CCC(c5ccc(NC6CCC(=O)NC6=O)cc5)CC4)CC3)cc2F)c2cn[nH]c2c1=O ; COc1cc(-c2cn(C)c(=O)c3[nH]ncc23)cc(OC)c1CN1CCN(C(=O)CCCN2CCC(c3ccc(NC4CCC(=O)NC4=O)cc3)CC2)CC1 ; COc1cc(-c2cn(C)c(=O)c3[nH]ncc23)cc(OC)c1CN1CCN(C(=O)CCCCCN2CCC(c3ccc(NC4CCC(=O)NC4=O)cc3)CC2)CC1 ; COc1cc(-c2cn(C)c(=O)c3[nH]ncc23)cc(OC)c1CN1CCN(C(=O)Cc2ccc(-c3ccc(NC4CCC(=O)NC4=O)cc3)cc2)CC1]

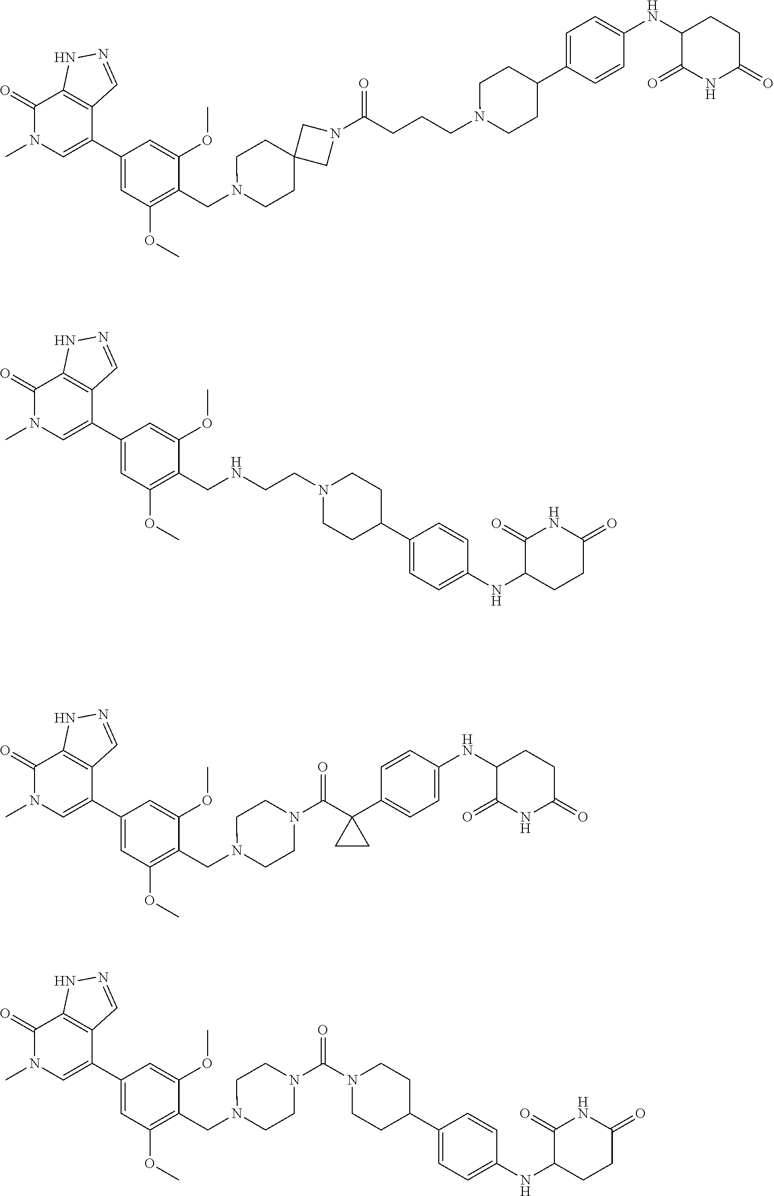 [SMILES: COc1cc(-c2cn(C)c(=O)c3[nH]ncc23)cc(OC)c1CN1CCC2(CC1)CN(C(=O)CCCN1CCC(c3ccc(NC4CCC(=O)NC4=O)cc3)CC1)C2 ; COc1cc(-c2cn(C)c(=O)c3[nH]ncc23)cc(OC)c1CNCCN1CCC(c2ccc(NC3CCC(=O)NC3=O)cc2)CC1 ; COc1cc(-c2cn(C)c(=O)c3[nH]ncc23)cc(OC)c1CN1CCN(C(=O)C2(c3ccc(NC4CCC(=O)NC4=O)cc3)CC2)CC1 ; COc1cc(-c2cn(C)c(=O)c3[nH]ncc23)cc(OC)c1CN1CCN(C(=O)N2CCC(c3ccc(NC4CCC(=O)NC4=O)cc3)CC2)CC1]

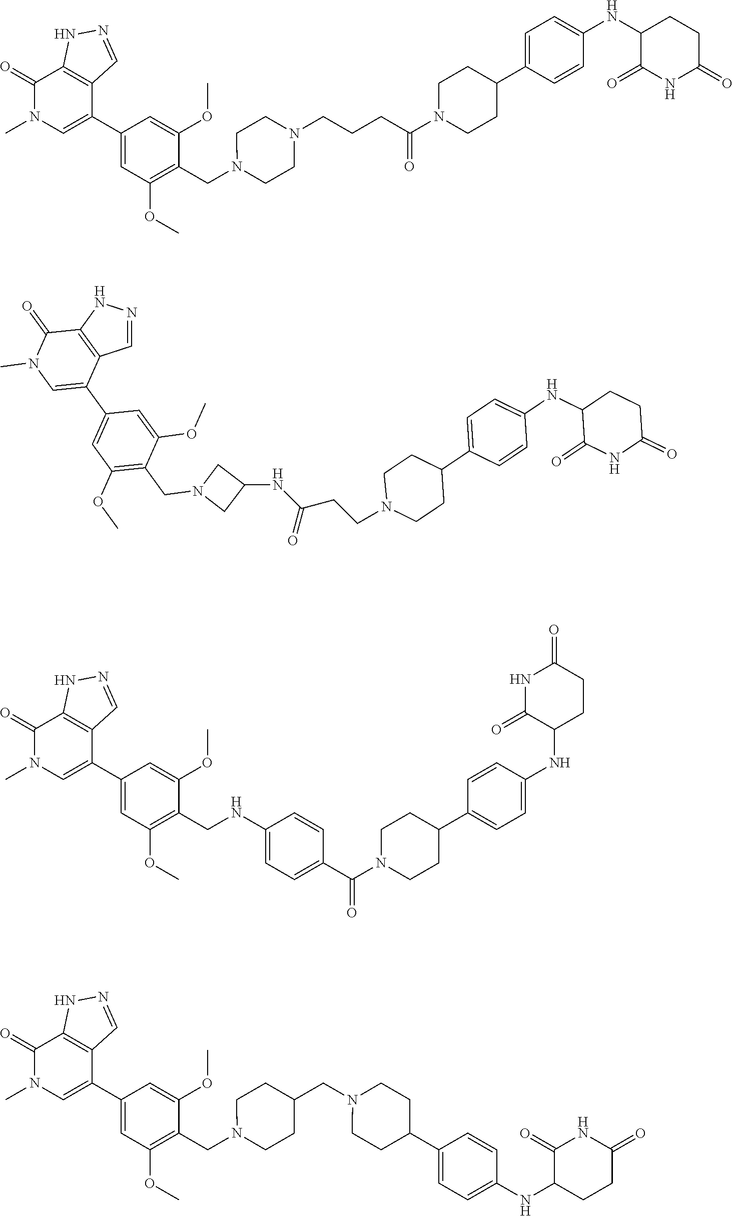 [SMILES: COc1cc(-c2cn(C)c(=O)c3[nH]ncc23)cc(OC)c1CN1CCN(CCCC(=O)N2CCC(c3ccc(NC4CCC(=O)NC4=O)cc3)CC2)CC1 ; COc1cc(-c2cn(C)c(=O)c3[nH]ncc23)cc(OC)c1CN1CC(NC(=O)CCN2CCC(c3ccc(NC4CCC(=O)NC4=O)cc3)CC2)C1 ; COc1cc(-c2cn(C)c(=O)c3[nH]ncc23)cc(OC)c1CNc1ccc(C(=O)N2CCC(c3ccc(NC4CCC(=O)NC4=O)cc3)CC2)cc1 ; COc1cc(-c2cn(C)c(=O)c3[nH]ncc23)cc(OC)c1CN1CCC(CN2CCC(c3ccc(NC4CCC(=O)NC4=O)cc3)CC2)CC1]

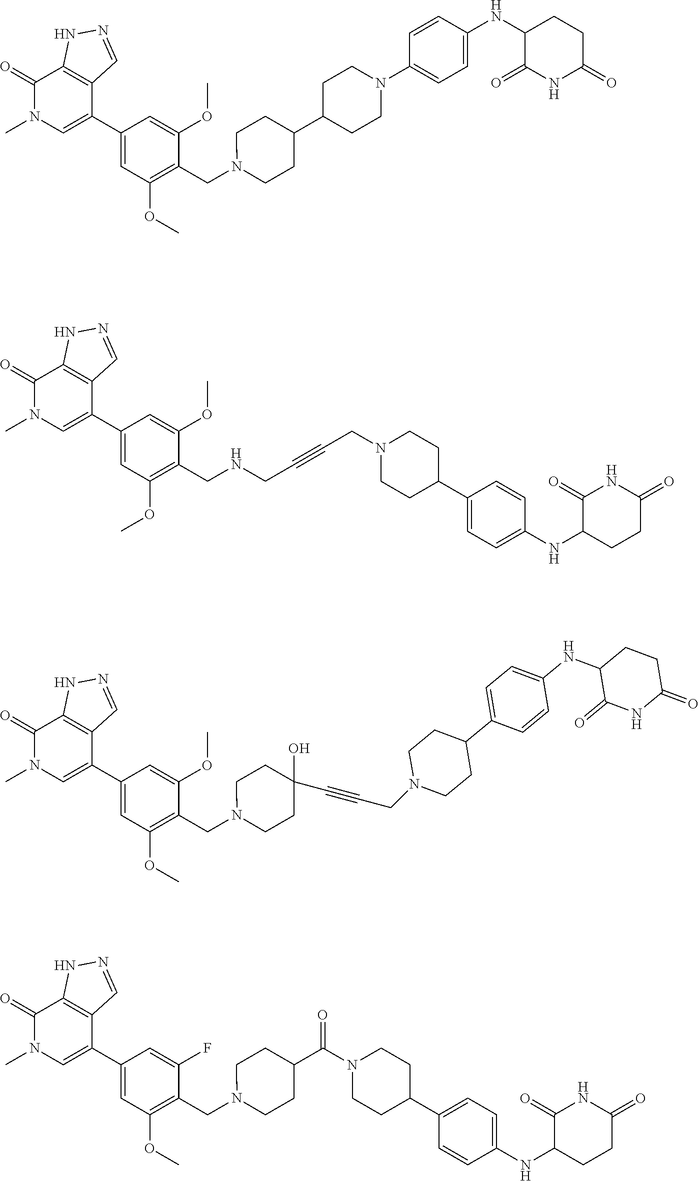 [SMILES: COc1cc(-c2cn(C)c(=O)c3[nH]ncc23)cc(OC)c1CN1CCC(C2CCN(c3ccc(NC4CCC(=O)NC4=O)cc3)CC2)CC1 ; COc1cc(-c2cn(C)c(=O)c3[nH]ncc23)cc(OC)c1CNCC#CCN1CCC(c2ccc(NC3CCC(=O)NC3=O)cc2)CC1 ; COc1cc(-c2cn(C)c(=O)c3[nH]ncc23)cc(OC)c1CN1CCC(O)(C#CCN2CCC(c3ccc(NC4CCC(=O)NC4=O)cc3)CC2)CC1 ; COc1cc(-c2cn(C)c(=O)c3[nH]ncc23)cc(F)c1CN1CCC(C(=O)N2CCC(c3ccc(NC4CCC(=O)NC4=O)cc3)CC2)CC1]

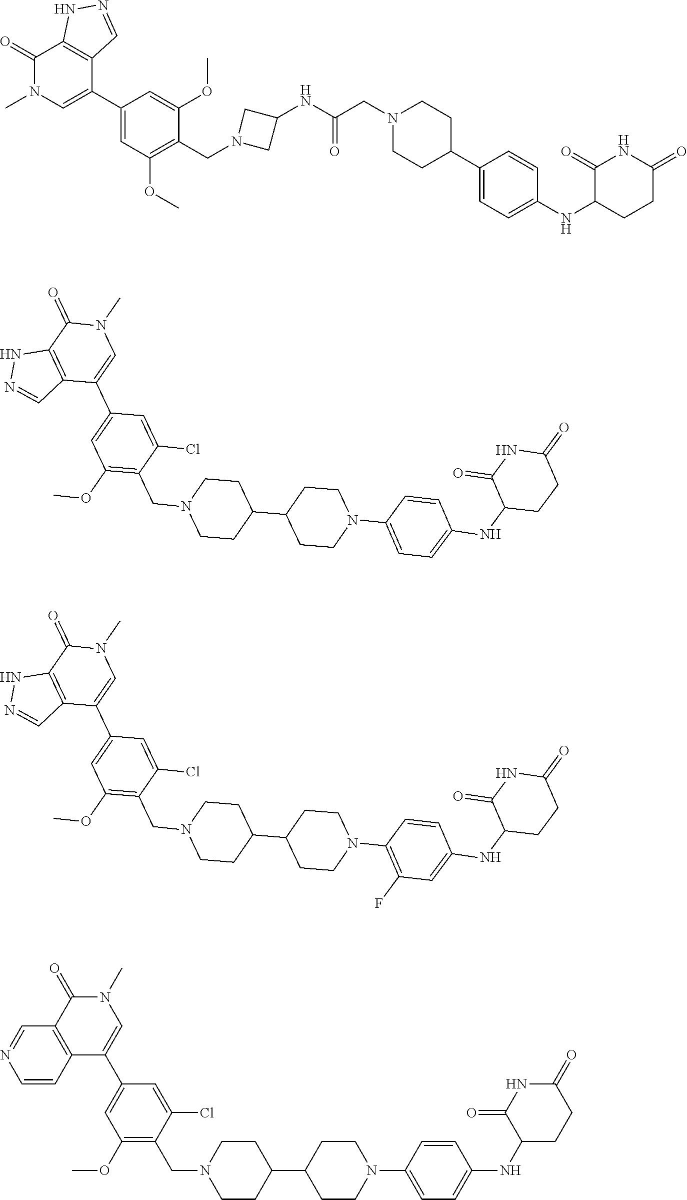 [SMILES: COc1cc(-c2cn(C)c(=O)c3[nH]ncc23)cc(OC)c1CN1CC(NC(=O)CN2CCC(c3ccc(NC4CCC(=O)NC4=O)cc3)CC2)C1 ; COc1cc(-c2cn(C)c(=O)c3[nH]ncc23)cc(Cl)c1CN1CCC(C2CCN(c3ccc(NC4CCC(=O)NC4=O)cc3)CC2)CC1 ; COc1cc(-c2cn(C)c(=O)c3[nH]ncc23)cc(Cl)c1CN1CCC(C2CCN(c3ccc(NC4CCC(=O)NC4=O)cc3F)CC2)CC1 ; COc1cc(-c2cn(C)c(=O)c3cnccc23)cc(Cl)c1CN1CCC(C2CCN(c3ccc(NC4CCC(=O)NC4=O)cc3)CC2)CC1]

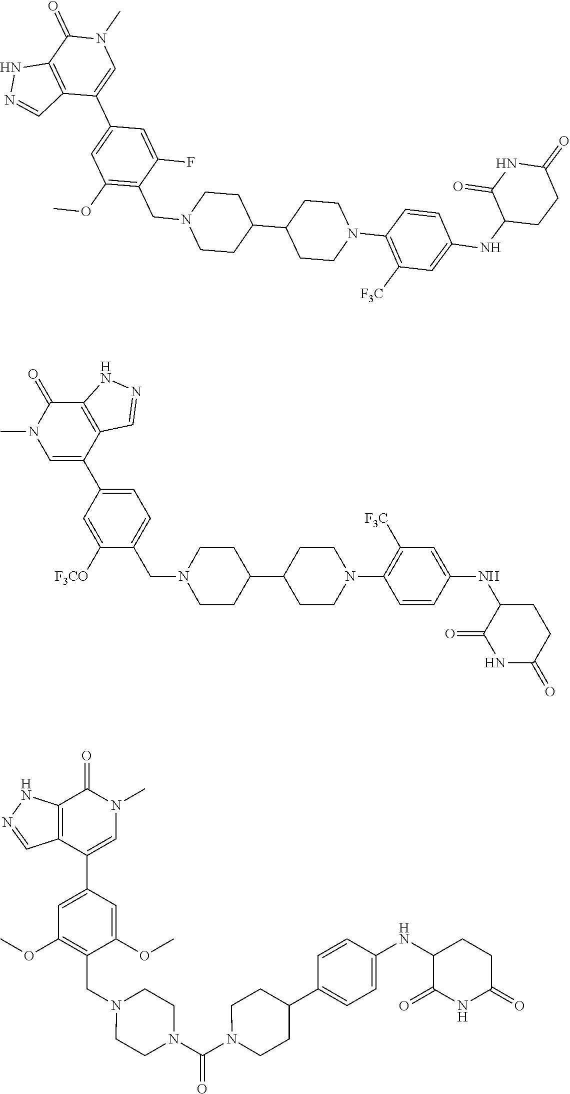 [SMILES: COc1cc(-c2cn(C)c(=O)c3[nH]ncc23)cc(F)c1CN1CCC(C2CCN(c3ccc(NC4CCC(=O)NC4=O)cc3C(F)(F)F)CC2)CC1 ; Cn1cc(-c2ccc(CN3CCC(C4CCN(c5ccc(NC6CCC(=O)NC6=O)cc5C(F)(F)F)CC4)CC3)c(OC(F)(F)F)c2)c2cn[nH]c2c1=O ; COc1cc(-c2cn(C)c(=O)c3[nH]ncc23)cc(OC)c1CN1CCN(C(=O)N2CCC(c3ccc(NC4CCC(=O)NC4=O)cc3)CC2)CC1]

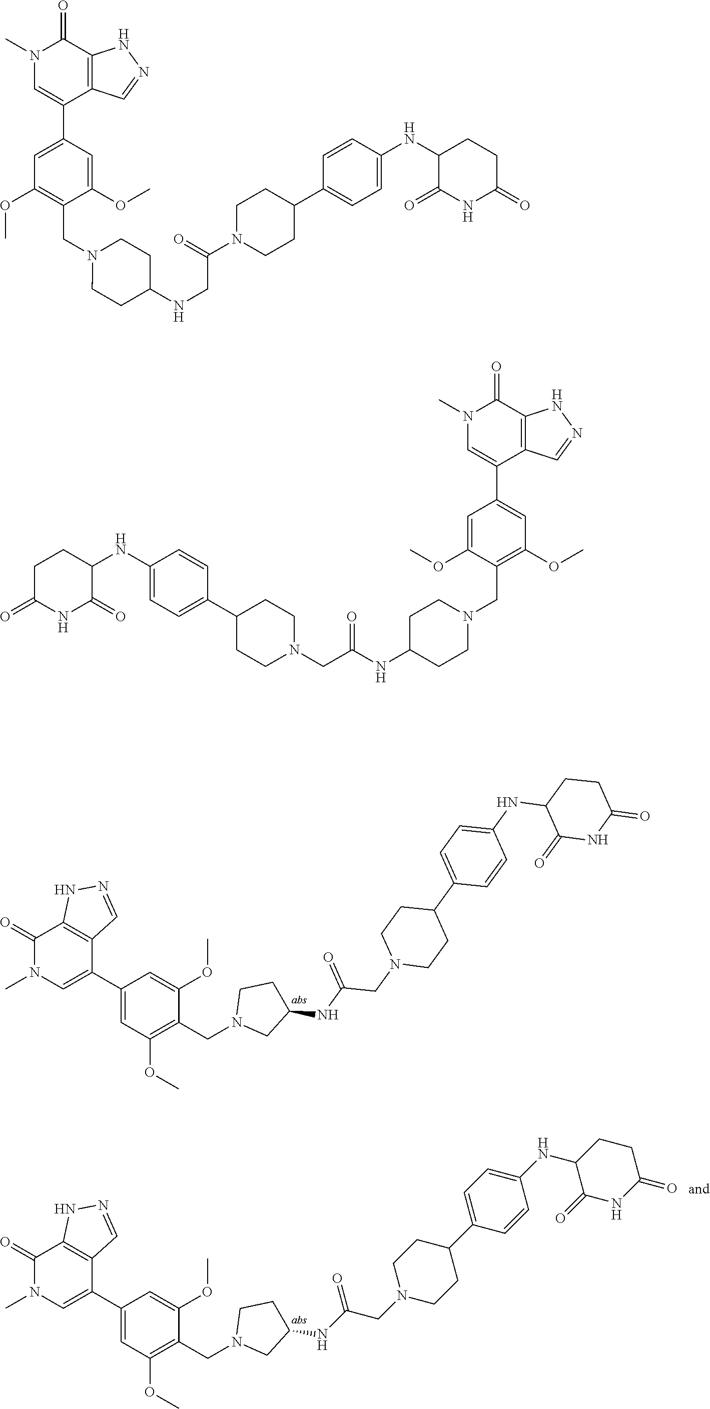

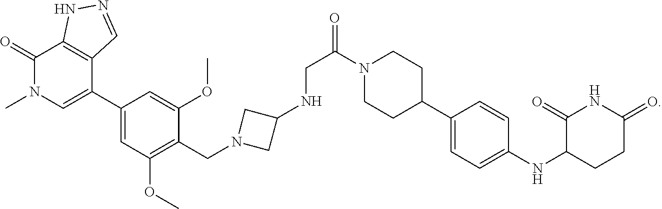 [SMILES: COc1cc(-c2cn(C)c(=O)c3[nH]ncc23)cc(OC)c1CN1CC(NCC(=O)N2CCC(c3ccc(NC4CCC(=O)NC4=O)cc3)CC2)C1]

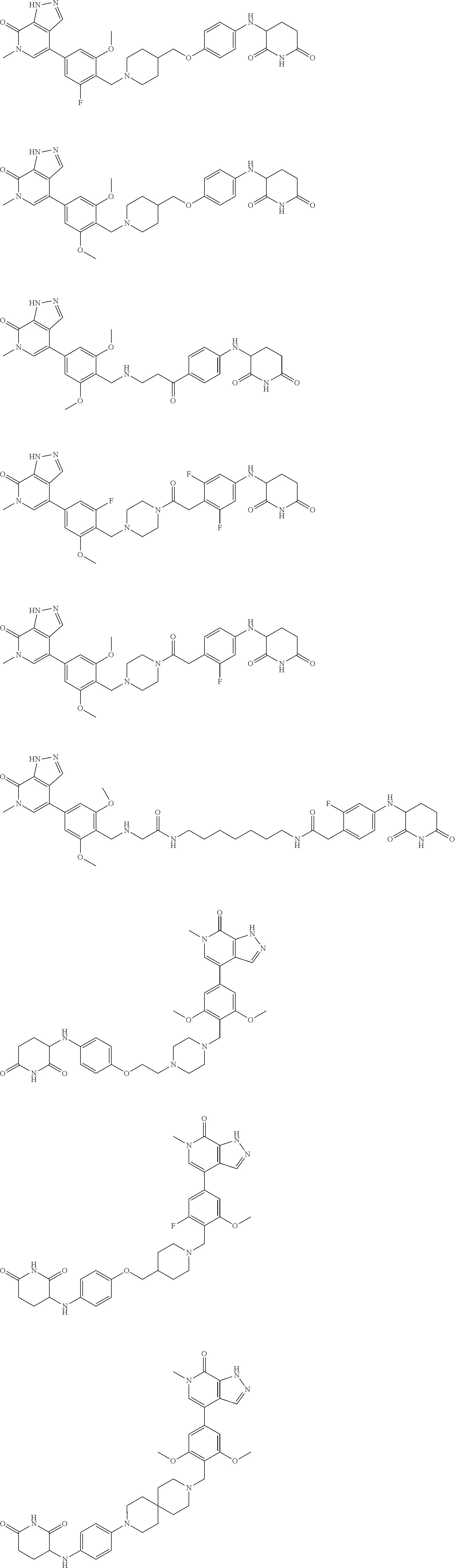

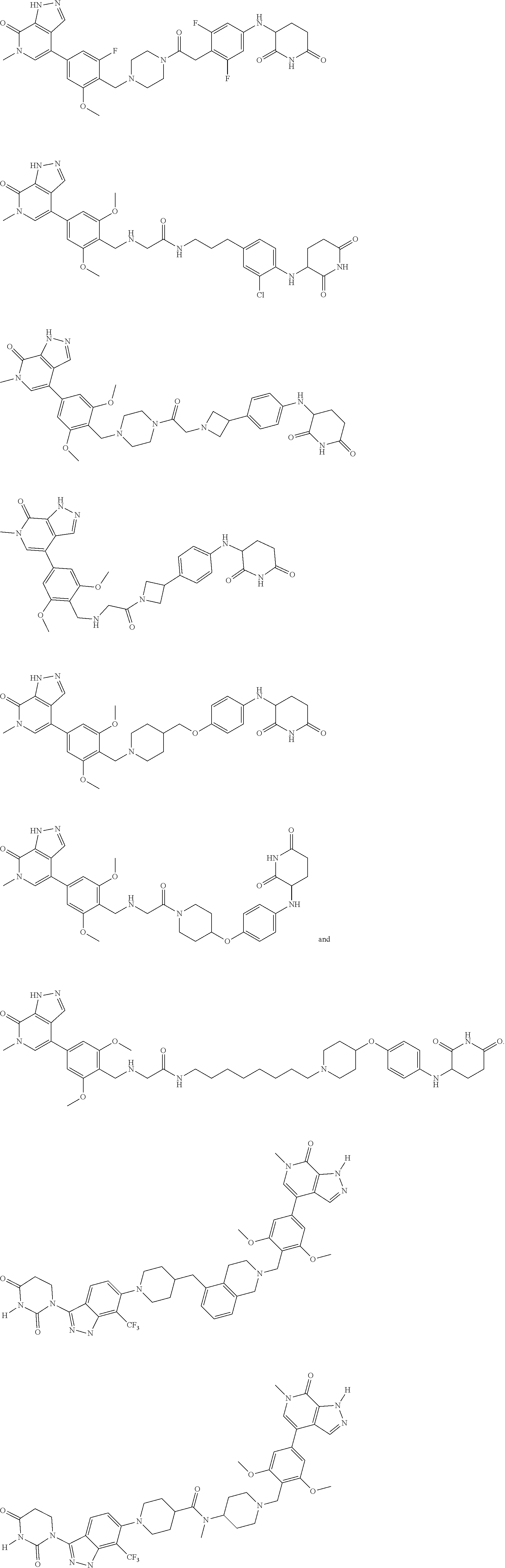

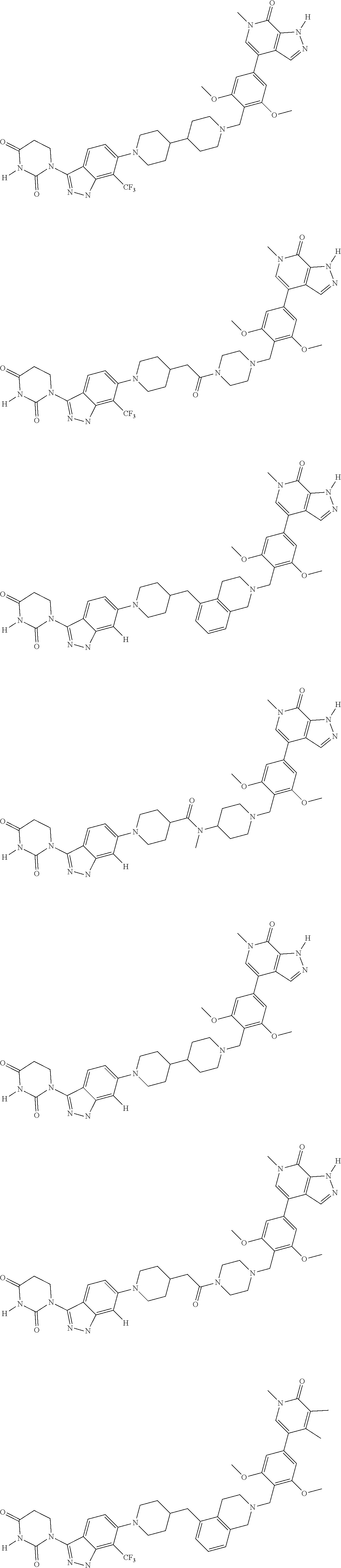

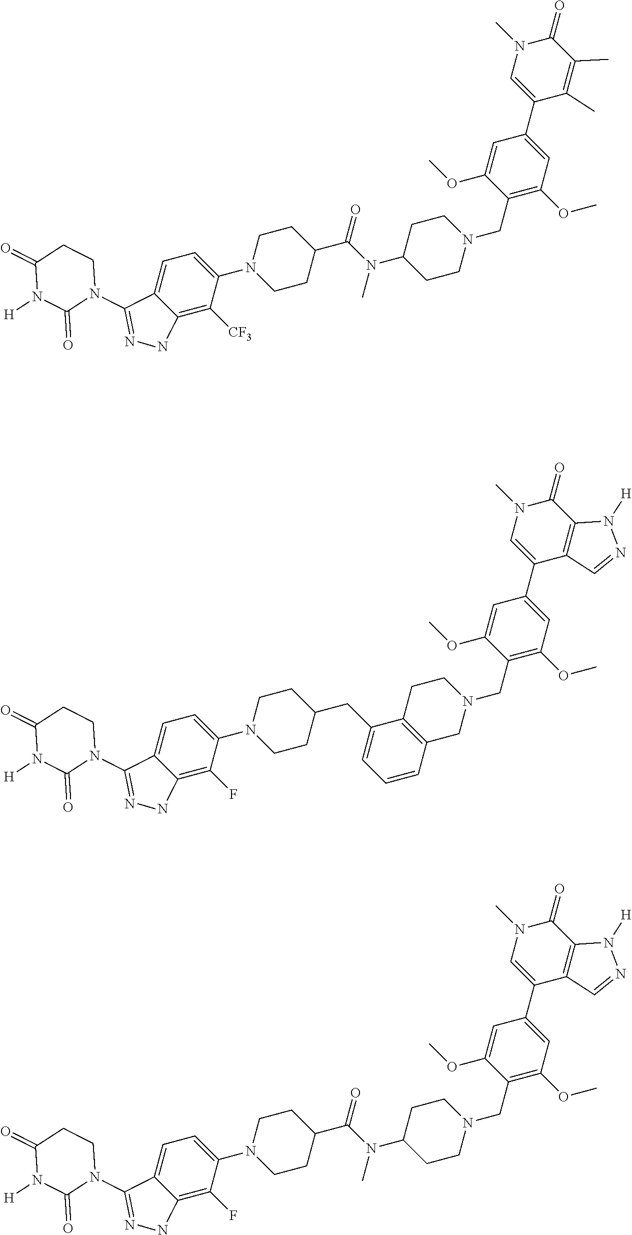 [SMILES: COc1cc(-c2cn(C)c(=O)c(C)c2C)cc(OC)c1CN1CCC(N(C)C(=O)C2CCN(c3ccc4c(N5CCC(=O)NC5=O)n[nH]c4c3C(F)(F)F)CC2)CC1 ; COc1cc(-c2cn(C)c(=O)c3[nH]ncc23)cc(OC)c1CN1CCc2c(CC3CCN(c4ccc5c(N6CCC(=O)NC6=O)n[nH]c5c4F)CC3)cccc2C1 ; COc1cc(-c2cn(C)c(=O)c3[nH]ncc23)cc(OC)c1CN1CCC(N(C)C(=O)C2CCN(c3ccc4c(N5CCC(=O)NC5=O)n[nH]c4c3F)CC2)CC1]

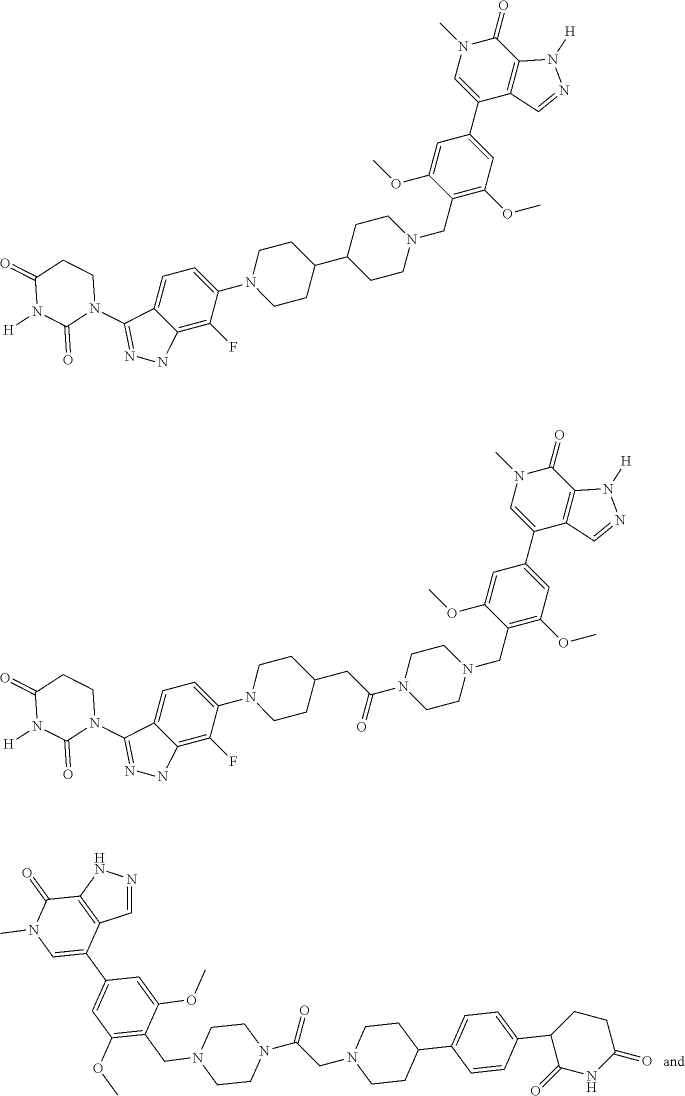 [SMILES: COc1cc(-c2cn(C)c(=O)c3[nH]ncc23)cc(OC)c1CN1CCC(C2CCN(c3ccc4c(N5CCC(=O)NC5=O)n[nH]c4c3F)CC2)CC1 ; COc1cc(-c2cn(C)c(=O)c3[nH]ncc23)cc(OC)c1CN1CCN(C(=O)CC2CCN(c3ccc4c(N5CCC(=O)NC5=O)n[nH]c4c3F)CC2)CC1 ; COc1cc(-c2cn(C)c(=O)c3[nH]ncc23)cc(OC)c1CN1CCN(C(=O)CN2CCC(c3ccc(C4CCC(=O)NC4=O)cc3)CC2)CC1 ; "and"]

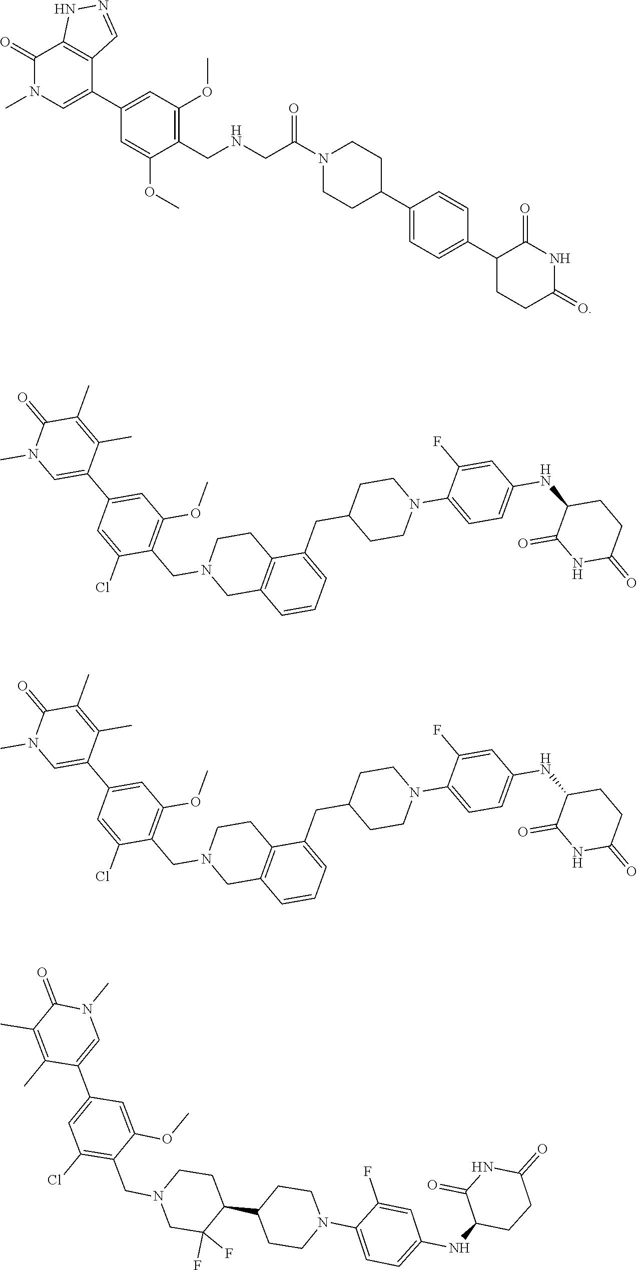 [SMILES: COc1cc(-c2cn(C)c(=O)c3[nH]ncc23)cc(OC)c1CNCC(=O)N1CCC(c2ccc(C3CCC(=O)NC3=O)cc2)CC1 ; COc1cc(-c2cn(C)c(=O)c(C)c2C)cc(Cl)c1CN1CCc2c(CC3CCN(c4ccc(N[C@H]5CCC(=O)NC5=O)cc4F)CC3)cccc2C1 ; COc1cc(-c2cn(C)c(=O)c(C)c2C)cc(Cl)c1CN1CCc2c(CC3CCN(c4ccc(N[C@@H]5CCC(=O)NC5=O)cc4F)CC3)cccc2C1 ; COc1cc(-c2cn(C)c(=O)c(C)c2C)cc(Cl)c1CN1CC[C@@H](C2CCN(c3ccc(N[C@@H]4CCC(=O)NC4=O)cc3F)CC2)C(F)(F)C1]

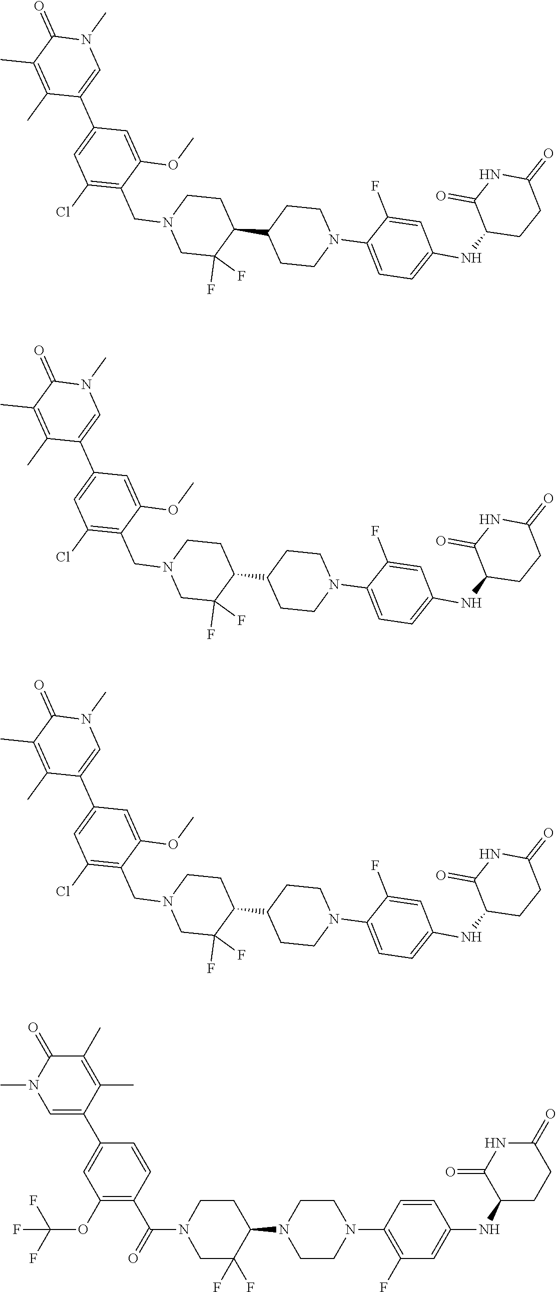 [SMILES: COc1cc(-c2cn(C)c(=O)c(C)c2C)cc(Cl)c1CN1CC[C@@H](C2CCN(c3ccc(N[C@H]4CCC(=O)NC4=O)cc3F)CC2)C(F)(F)C1 ; COc1cc(-c2cn(C)c(=O)c(C)c2C)cc(Cl)c1CN1CC[C@H](C2CCN(c3ccc(N[C@@H]4CCC(=O)NC4=O)cc3F)CC2)C(F)(F)C1 ; COc1cc(-c2cn(C)c(=O)c(C)c2C)cc(Cl)c1CN1CC[C@H](C2CCN(c3ccc(N[C@H]4CCC(=O)NC4=O)cc3F)CC2)C(F)(F)C1 ; Cc1c(-c2ccc(C(=O)N3CC[C@@H](N4CCN(c5ccc(N[C@@H]6CCC(=O)NC6=O)cc5F)CC4)C(F)(F)C3)c(OC(F)(F)F)c2)cn(C)c(=O)c1C]

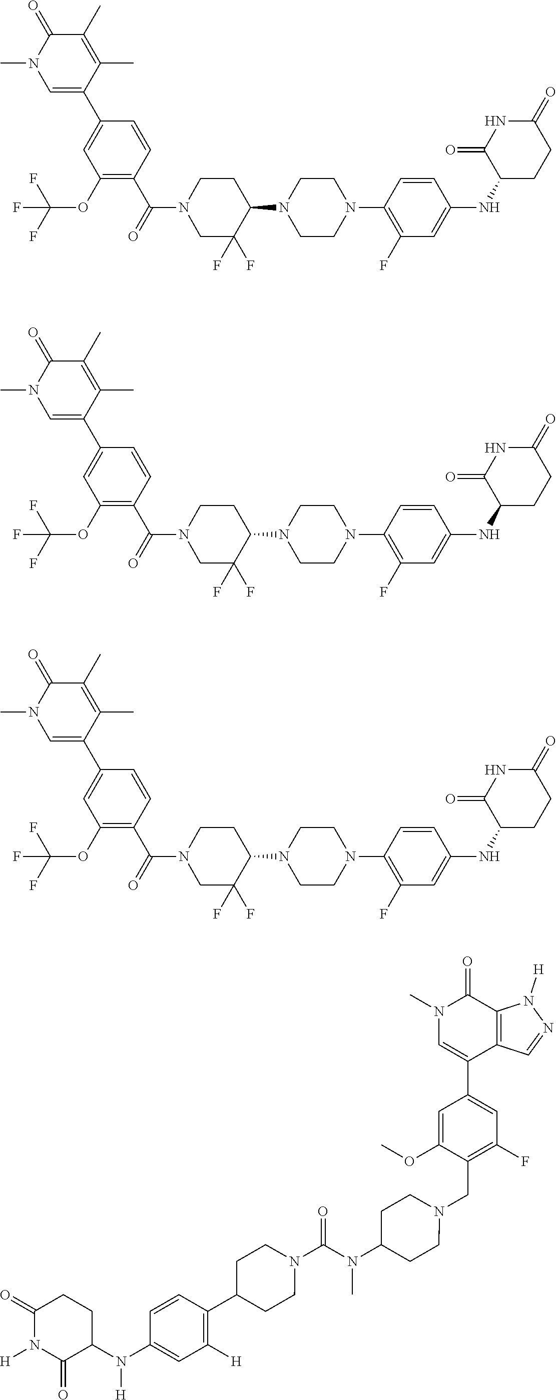 [SMILES: Cc1c(-c2ccc(C(=O)N3CC[C@@H](N4CCN(c5ccc(N[C@H]6CCC(=O)NC6=O)cc5F)CC4)C(F)(F)C3)c(OC(F)(F)F)c2)cn(C)c(=O)c1C ; Cc1c(-c2ccc(C(=O)N3CC[C@H](N4CCN(c5ccc(N[C@@H]6CCC(=O)NC6=O)cc5F)CC4)C(F)(F)C3)c(OC(F)(F)F)c2)cn(C)c(=O)c1C ; Cc1c(-c2ccc(C(=O)N3CC[C@H](N4CCN(c5ccc(N[C@H]6CCC(=O)NC6=O)cc5F)CC4)C(F)(F)C3)c(OC(F)(F)F)c2)cn(C)c(=O)c1C ; COc1cc(-c2cn(C)c(=O)c3[nH]ncc23)cc(F)c1CN1CCC(N(C)C(=O)N2CCC(c3ccc(NC4CCC(=O)NC4=O)cc3)CC2)CC1]

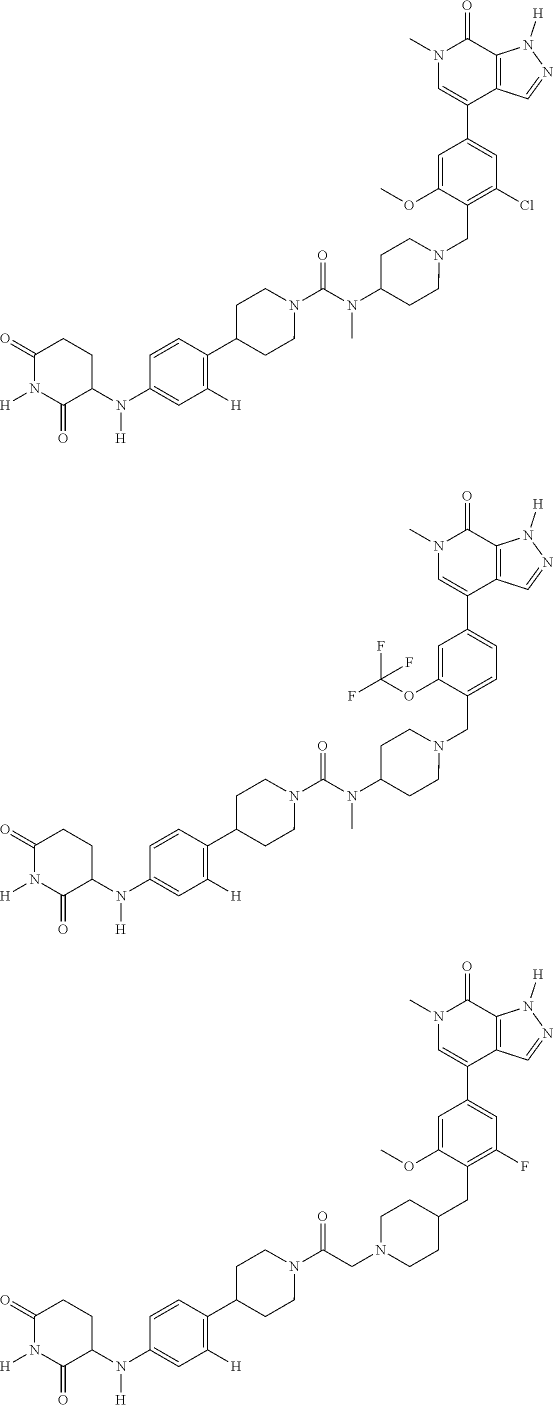 [SMILES: COc1cc(-c2cn(C)c(=O)c3[nH]ncc23)cc(Cl)c1CN1CCC(N(C)C(=O)N2CCC(c3ccc(NC4CCC(=O)NC4=O)cc3)CC2)CC1 ; CN(C(=O)N1CCC(c2ccc(NC3CCC(=O)NC3=O)cc2)CC1)C1CCN(Cc2ccc(-c3cn(C)c(=O)c4[nH]ncc34)cc2OC(F)(F)F)CC1 ; COc1cc(-c2cn(C)c(=O)c3[nH]ncc23)cc(F)c1CC1CCN(CC(=O)N2CCC(c3ccc(NC4CCC(=O)NC4=O)cc3)CC2)CC1]

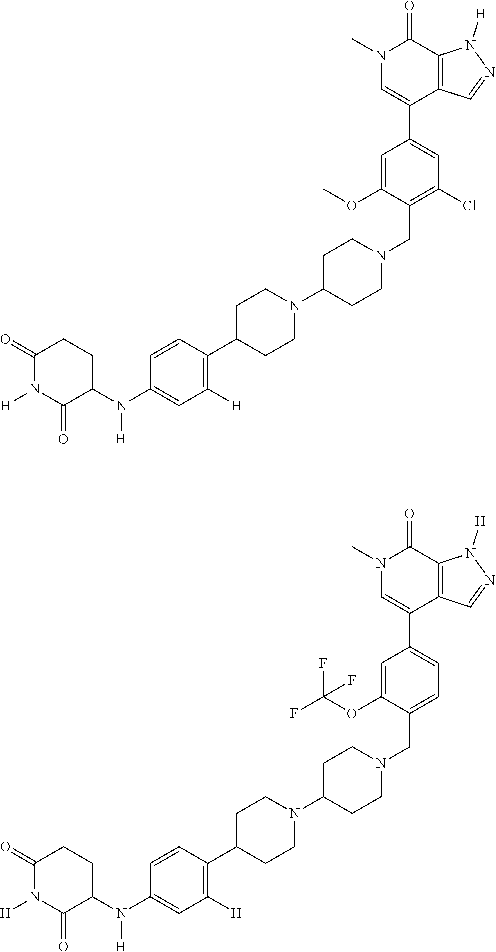 [SMILES: COc1cc(-c2cn(C)c(=O)c3[nH]ncc23)cc(Cl)c1CN1CCC(N2CCC(c3ccc(NC4CCC(=O)NC4=O)cc3)CC2)CC1 ; Cn1cc(-c2ccc(CN3CCC(N4CCC(c5ccc(NC6CCC(=O)NC6=O)cc5)CC4)CC3)c(OC(F)(F)F)c2)c2cn[nH]c2c1=O]

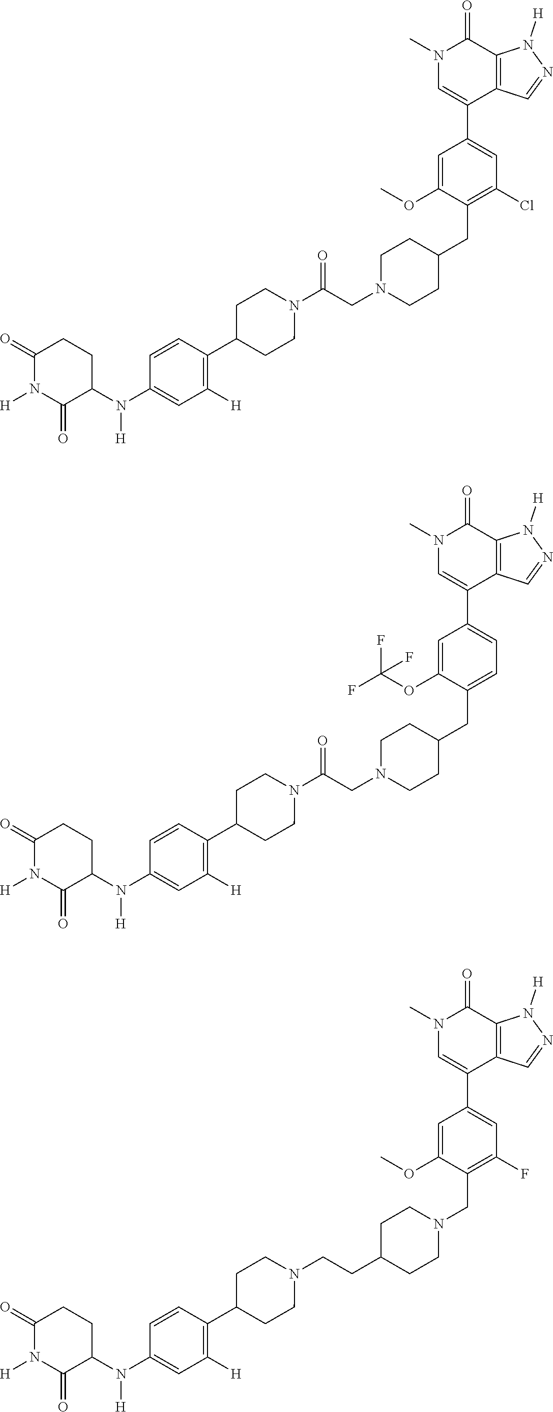 [SMILES: COc1cc(-c2cn(C)c(=O)c3[nH]ncc23)cc(Cl)c1CC1CCN(CC(=O)N2CCC(c3ccc(NC4CCC(=O)NC4=O)cc3)CC2)CC1 ; Cn1cc(-c2ccc(CC3CCN(CC(=O)N4CCC(c5ccc(NC6CCC(=O)NC6=O)cc5)CC4)CC3)c(OC(F)(F)F)c2)c2cn[nH]c2c1=O ; COc1cc(-c2cn(C)c(=O)c3[nH]ncc23)cc(F)c1CN1CCC(CCN2CCC(c3ccc(NC4CCC(=O)NC4=O)cc3)CC2)CC1]

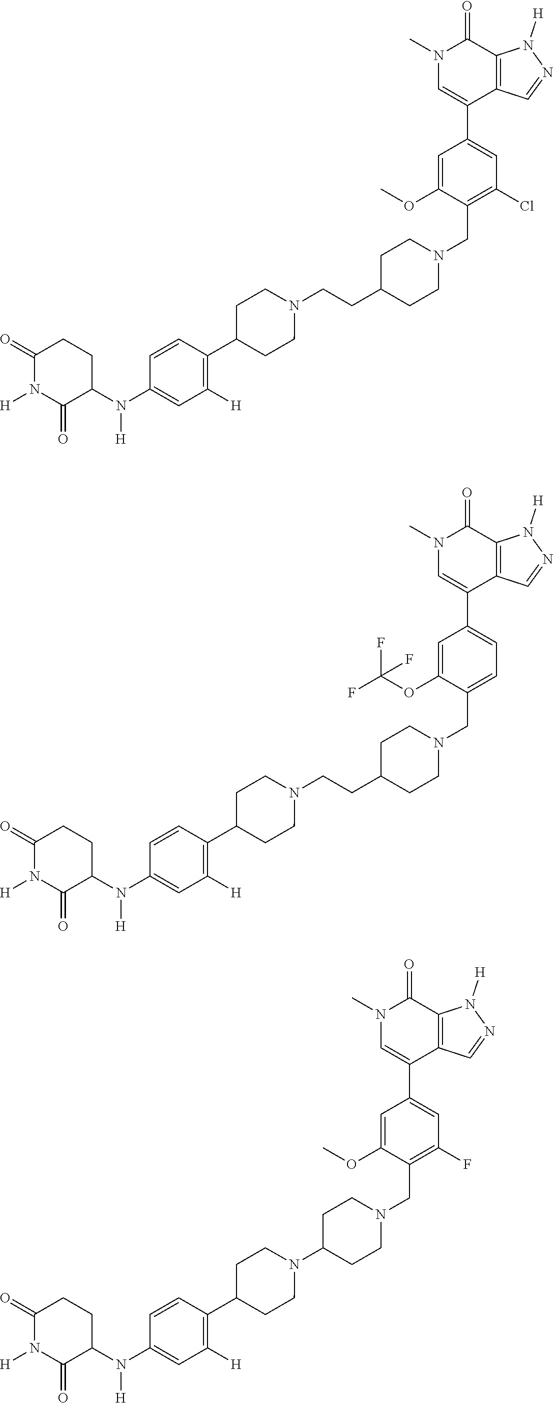 [SMILES: COc1cc(-c2cn(C)c(=O)c3[nH]ncc23)cc(Cl)c1CN1CCC(CCN2CCC(c3ccc(NC4CCC(=O)NC4=O)cc3)CC2)CC1 ; Cn1cc(-c2ccc(CN3CCC(CCN4CCC(c5ccc(NC6CCC(=O)NC6=O)cc5)CC4)CC3)c(OC(F)(F)F)c2)c2cn[nH]c2c1=O ; COc1cc(-c2cn(C)c(=O)c3[nH]ncc23)cc(F)c1CN1CCC(N2CCC(c3ccc(NC4CCC(=O)NC4=O)cc3)CC2)CC1]

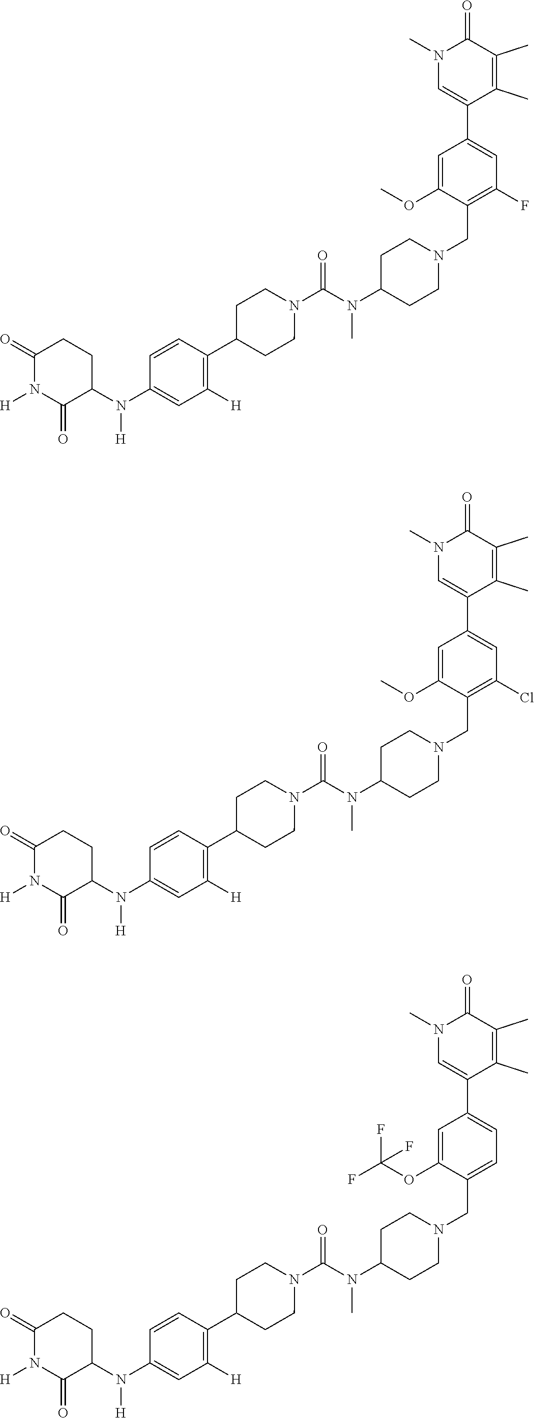 [SMILES: COc1cc(-c2cn(C)c(=O)c(C)c2C)cc(F)c1CN1CCC(N(C)C(=O)N2CCC(c3ccc(NC4CCC(=O)NC4=O)cc3)CC2)CC1 ; COc1cc(-c2cn(C)c(=O)c(C)c2C)cc(Cl)c1CN1CCC(N(C)C(=O)N2CCC(c3ccc(NC4CCC(=O)NC4=O)cc3)CC2)CC1 ; Cc1c(-c2ccc(CN3CCC(N(C)C(=O)N4CCC(c5ccc(NC6CCC(=O)NC6=O)cc5)CC4)CC3)c(OC(F)(F)F)c2)cn(C)c(=O)c1C]

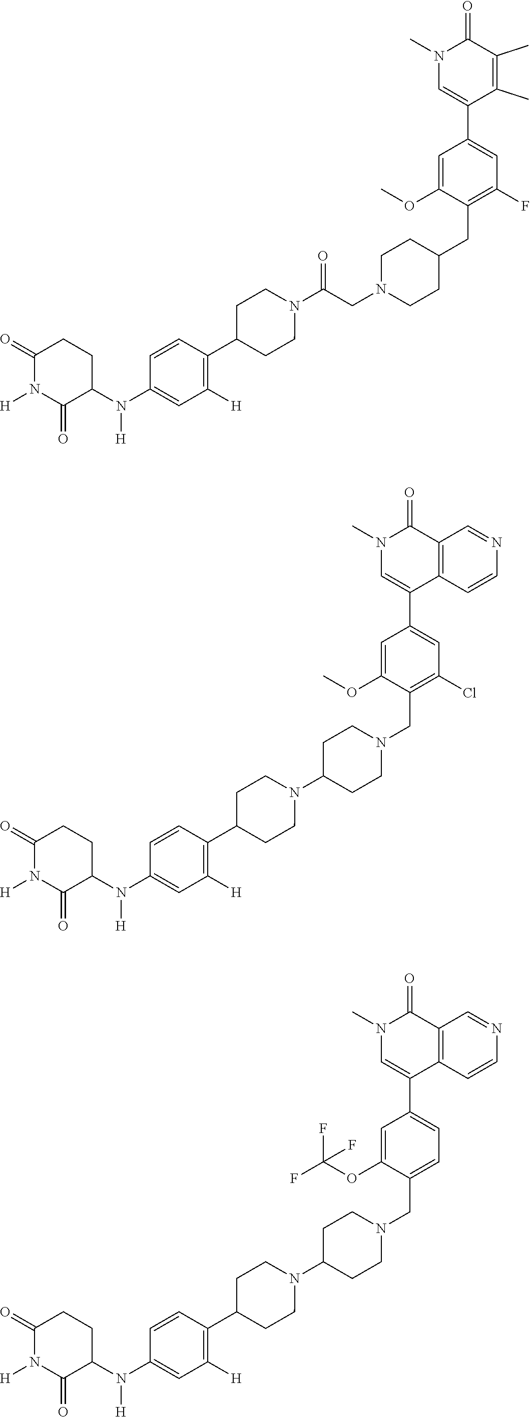 [SMILES: COc1cc(-c2cn(C)c(=O)c(C)c2C)cc(F)c1CC1CCN(CC(=O)N2CCC(c3ccc(NC4CCC(=O)NC4=O)cc3)CC2)CC1 ; COc1cc(-c2cn(C)c(=O)c3cnccc23)cc(Cl)c1CN1CCC(N2CCC(c3ccc(NC4CCC(=O)NC4=O)cc3)CC2)CC1 ; Cn1cc(-c2ccc(CN3CCC(N4CCC(c5ccc(NC6CCC(=O)NC6=O)cc5)CC4)CC3)c(OC(F)(F)F)c2)c2ccncc2c1=O]

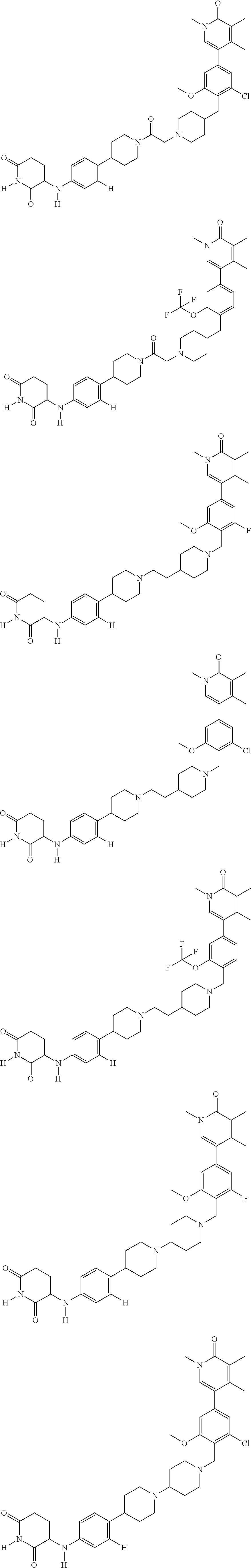

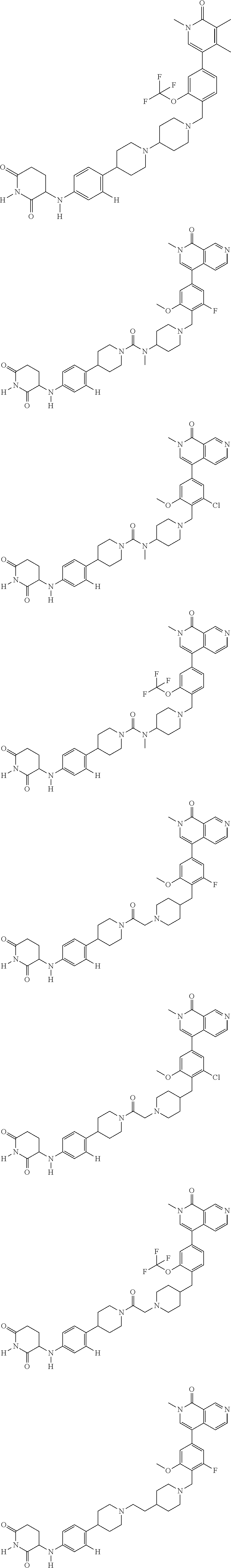

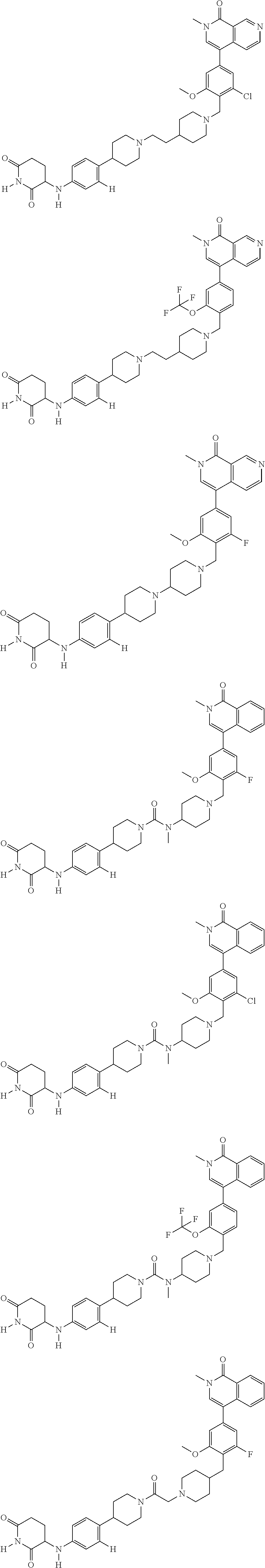

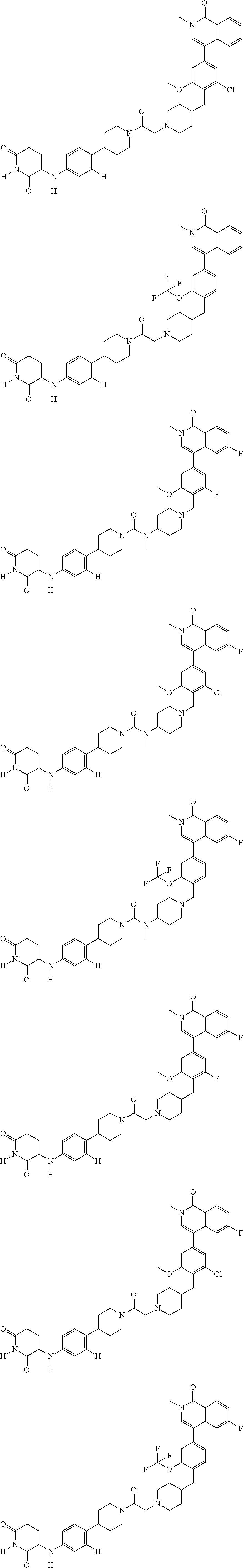

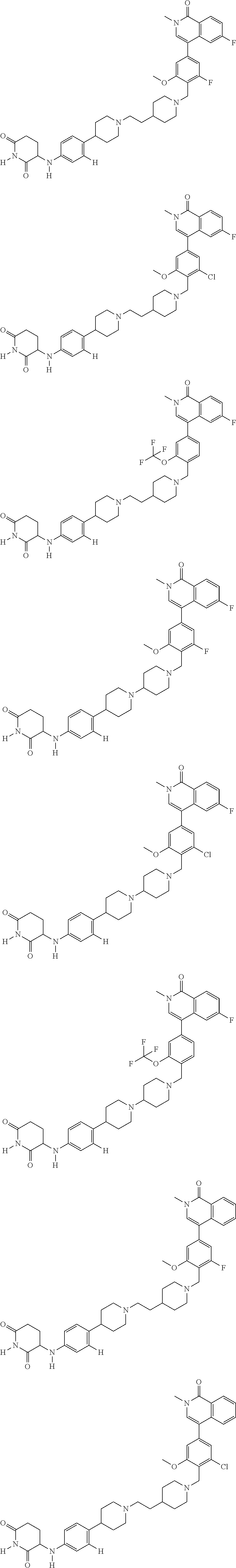

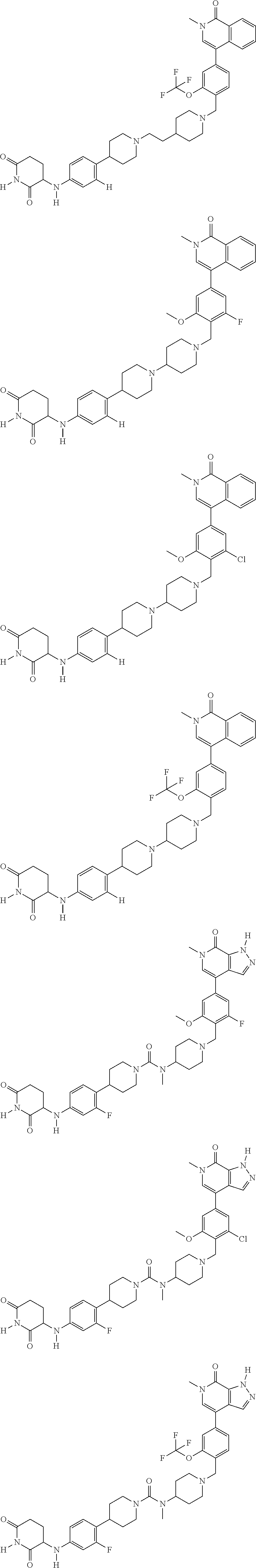

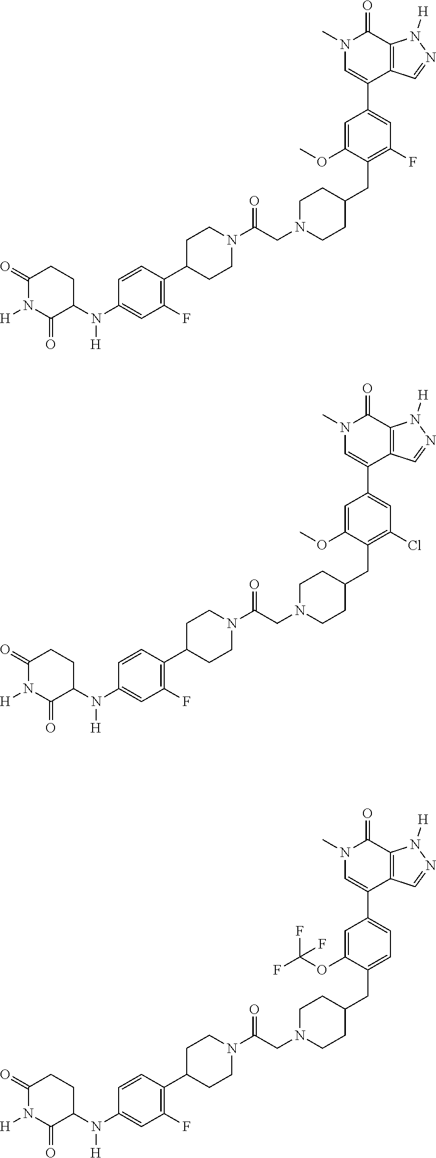 [SMILES: COc1cc(-c2cn(C)c(=O)c3[nH]ncc23)cc(F)c1CC1CCN(CC(=O)N2CCC(c3ccc(NC4CCC(=O)NC4=O)cc3F)CC2)CC1 ; COc1cc(-c2cn(C)c(=O)c3[nH]ncc23)cc(Cl)c1CC1CCN(CC(=O)N2CCC(c3ccc(NC4CCC(=O)NC4=O)cc3F)CC2)CC1 ; Cn1cc(-c2ccc(CC3CCN(CC(=O)N4CCC(c5ccc(NC6CCC(=O)NC6=O)cc5F)CC4)CC3)c(OC(F)(F)F)c2)c2cn[nH]c2c1=O]

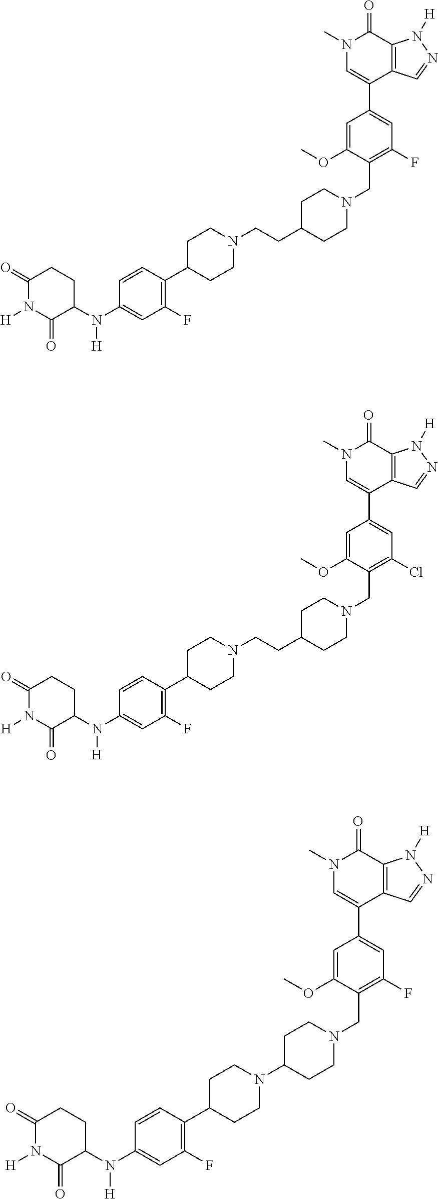 [SMILES: COc1cc(-c2cn(C)c(=O)c3[nH]ncc23)cc(F)c1CN1CCC(CCN2CCC(c3ccc(NC4CCC(=O)NC4=O)cc3F)CC2)CC1 ; COc1cc(-c2cn(C)c(=O)c3[nH]ncc23)cc(Cl)c1CN1CCC(CCN2CCC(c3ccc(NC4CCC(=O)NC4=O)cc3F)CC2)CC1 ; COc1cc(-c2cn(C)c(=O)c3[nH]ncc23)cc(F)c1CN1CCC(N2CCC(c3ccc(NC4CCC(=O)NC4=O)cc3F)CC2)CC1]

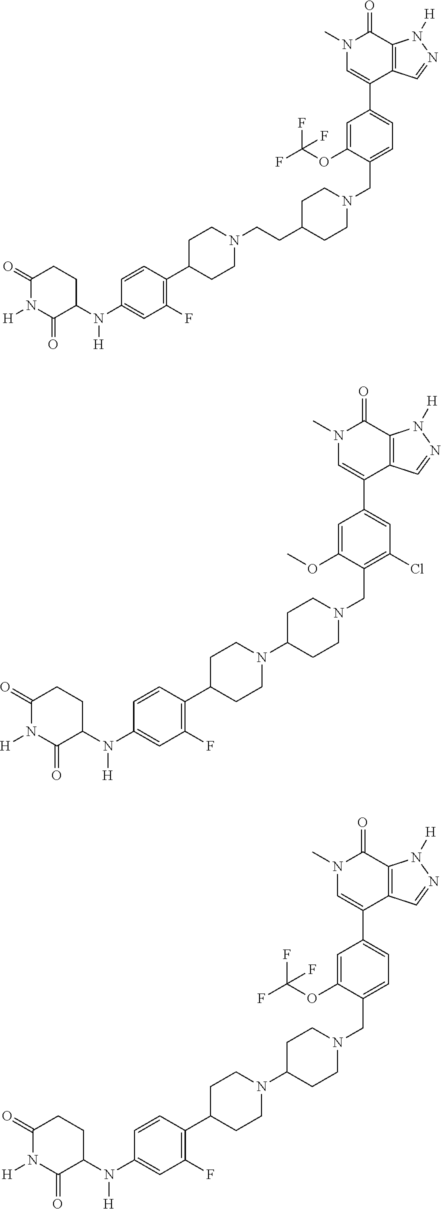 [SMILES: Cn1cc(-c2ccc(CN3CCC(CCN4CCC(c5ccc(NC6CCC(=O)NC6=O)cc5F)CC4)CC3)c(OC(F)(F)F)c2)c2cn[nH]c2c1=O ; COc1cc(-c2cn(C)c(=O)c3[nH]ncc23)cc(Cl)c1CN1CCC(N2CCC(c3ccc(NC4CCC(=O)NC4=O)cc3F)CC2)CC1 ; Cn1cc(-c2ccc(CN3CCC(N4CCC(c5ccc(NC6CCC(=O)NC6=O)cc5F)CC4)CC3)c(OC(F)(F)F)c2)c2cn[nH]c2c1=O]

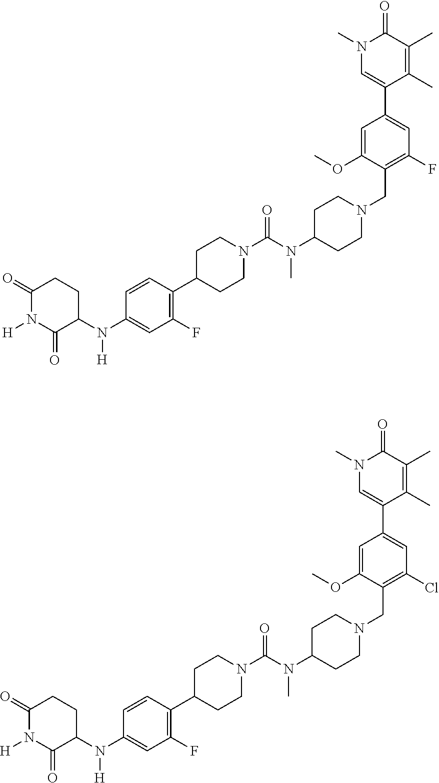 [SMILES: COc1cc(-c2cn(C)c(=O)c(C)c2C)cc(F)c1CN1CCC(N(C)C(=O)N2CCC(c3ccc(NC4CCC(=O)NC4=O)cc3F)CC2)CC1 ; COc1cc(-c2cn(C)c(=O)c(C)c2C)cc(Cl)c1CN1CCC(N(C)C(=O)N2CCC(c3ccc(NC4CCC(=O)NC4=O)cc3F)CC2)CC1]

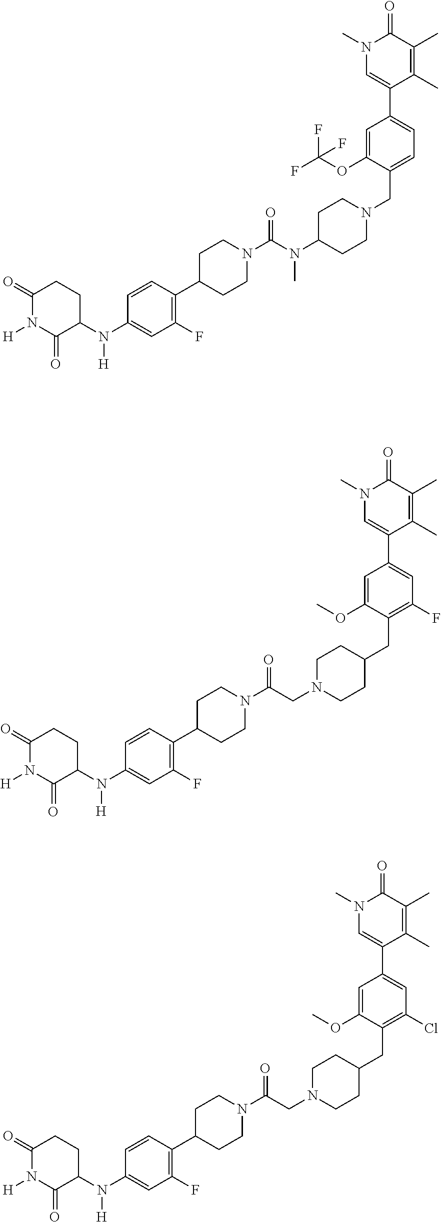 [SMILES: Cc1c(-c2ccc(CN3CCC(N(C)C(=O)N4CCC(c5ccc(NC6CCC(=O)NC6=O)cc5F)CC4)CC3)c(OC(F)(F)F)c2)cn(C)c(=O)c1C ; COc1cc(-c2cn(C)c(=O)c(C)c2C)cc(F)c1CC1CCN(CC(=O)N2CCC(c3ccc(NC4CCC(=O)NC4=O)cc3F)CC2)CC1 ; COc1cc(-c2cn(C)c(=O)c(C)c2C)cc(Cl)c1CC1CCN(CC(=O)N2CCC(c3ccc(NC4CCC(=O)NC4=O)cc3F)CC2)CC1]

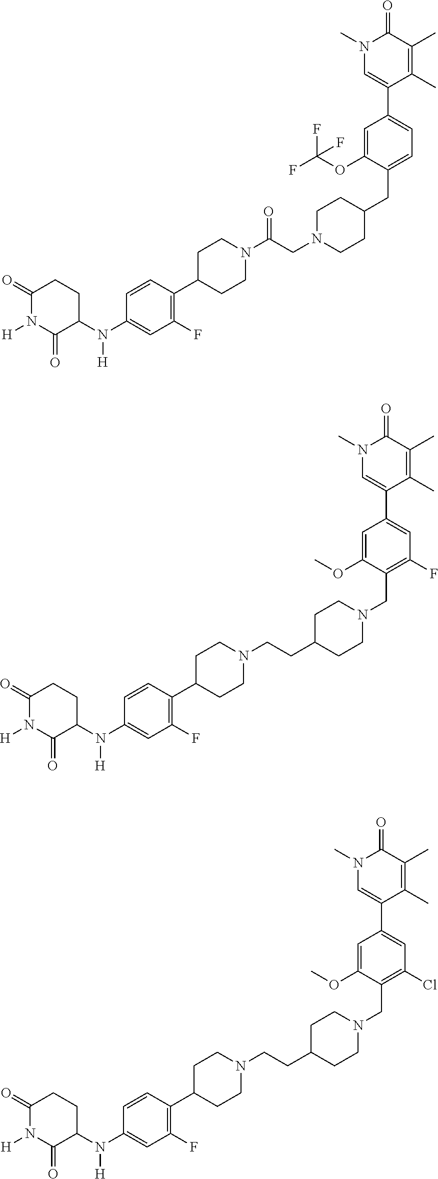 [SMILES: Cc1c(-c2ccc(CC3CCN(CC(=O)N4CCC(c5ccc(NC6CCC(=O)NC6=O)cc5F)CC4)CC3)c(OC(F)(F)F)c2)cn(C)c(=O)c1C ; COc1cc(-c2cn(C)c(=O)c(C)c2C)cc(F)c1CN1CCC(CCN2CCC(c3ccc(NC4CCC(=O)NC4=O)cc3F)CC2)CC1 ; COc1cc(-c2cn(C)c(=O)c(C)c2C)cc(Cl)c1CN1CCC(CCN2CCC(c3ccc(NC4CCC(=O)NC4=O)cc3F)CC2)CC1]

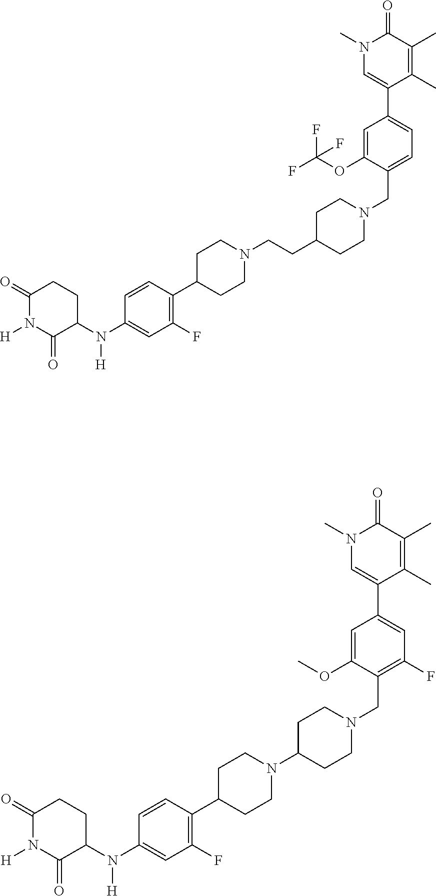 [SMILES: Cc1c(-c2ccc(CN3CCC(CCN4CCC(c5ccc(NC6CCC(=O)NC6=O)cc5F)CC4)CC3)c(OC(F)(F)F)c2)cn(C)c(=O)c1C ; COc1cc(-c2cn(C)c(=O)c(C)c2C)cc(F)c1CN1CCC(N2CCC(c3ccc(NC4CCC(=O)NC4=O)cc3F)CC2)CC1]

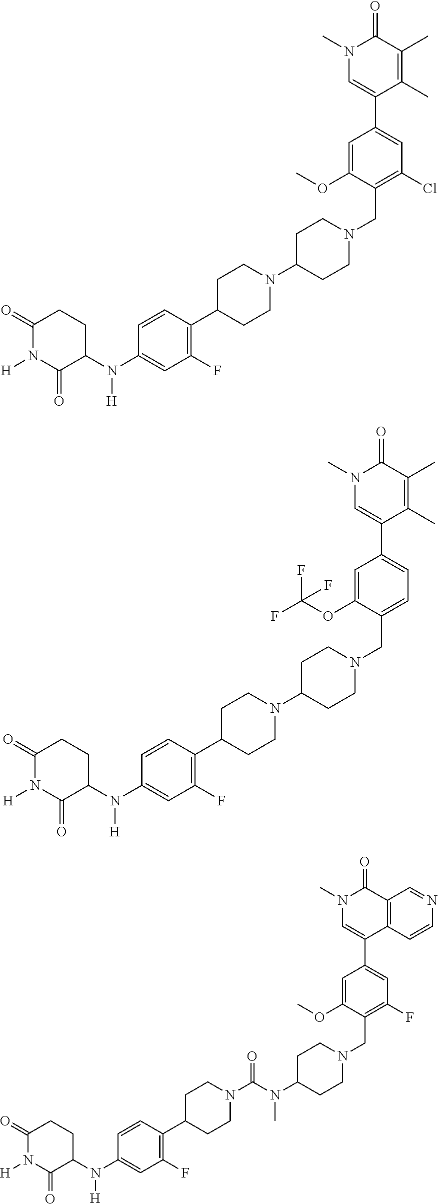 [SMILES: COc1cc(-c2cn(C)c(=O)c(C)c2C)cc(Cl)c1CN1CCC(N2CCC(c3ccc(NC4CCC(=O)NC4=O)cc3F)CC2)CC1 ; Cc1c(-c2ccc(CN3CCC(N4CCC(c5ccc(NC6CCC(=O)NC6=O)cc5F)CC4)CC3)c(OC(F)(F)F)c2)cn(C)c(=O)c1C ; COc1cc(-c2cn(C)c(=O)c3cnccc23)cc(F)c1CN1CCC(N(C)C(=O)N2CCC(c3ccc(NC4CCC(=O)NC4=O)cc3F)CC2)CC1]

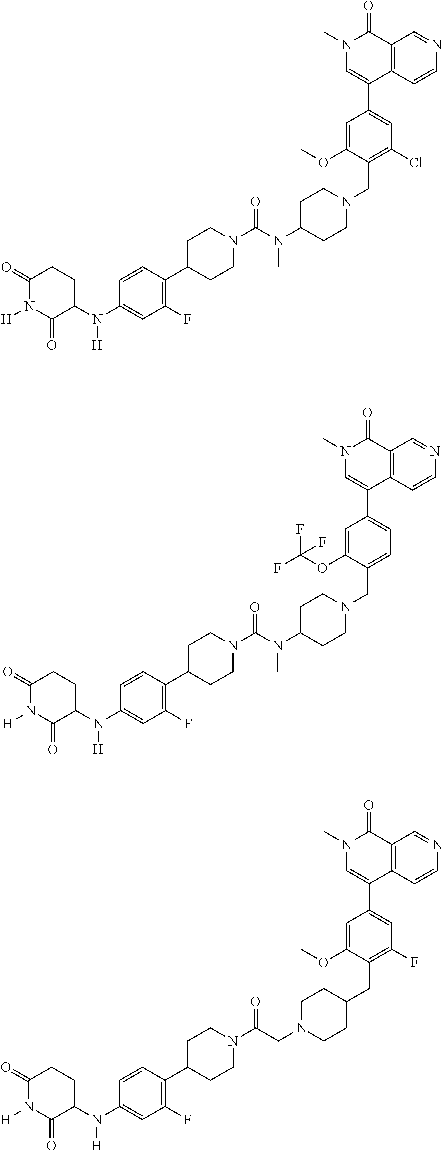 [SMILES: COc1cc(-c2cn(C)c(=O)c3cnccc23)cc(Cl)c1CN1CCC(N(C)C(=O)N2CCC(c3ccc(NC4CCC(=O)NC4=O)cc3F)CC2)CC1 ; CN(C(=O)N1CCC(c2ccc(NC3CCC(=O)NC3=O)cc2F)CC1)C1CCN(Cc2ccc(-c3cn(C)c(=O)c4cnccc34)cc2OC(F)(F)F)CC1 ; COc1cc(-c2cn(C)c(=O)c3cnccc23)cc(F)c1CC1CCN(CC(=O)N2CCC(c3ccc(NC4CCC(=O)NC4=O)cc3F)CC2)CC1]

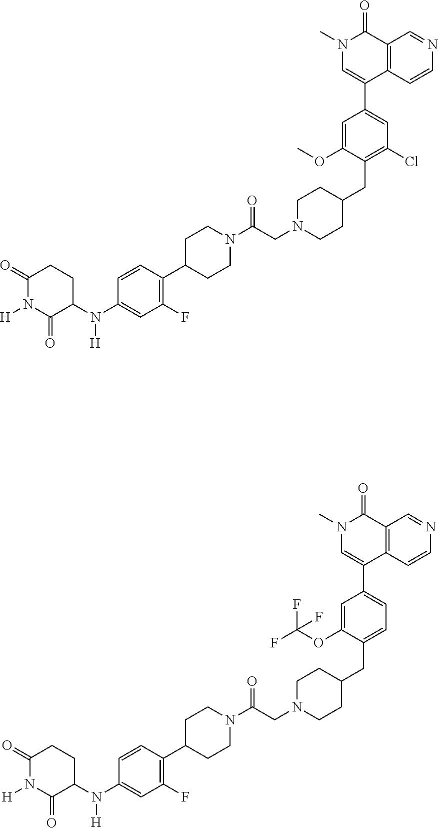 [SMILES: COc1cc(-c2cn(C)c(=O)c3cnccc23)cc(Cl)c1CC1CCN(CC(=O)N2CCC(c3ccc(NC4CCC(=O)NC4=O)cc3F)CC2)CC1 ; Cn1cc(-c2ccc(CC3CCN(CC(=O)N4CCC(c5ccc(NC6CCC(=O)NC6=O)cc5F)CC4)CC3)c(OC(F)(F)F)c2)c2ccncc2c1=O]

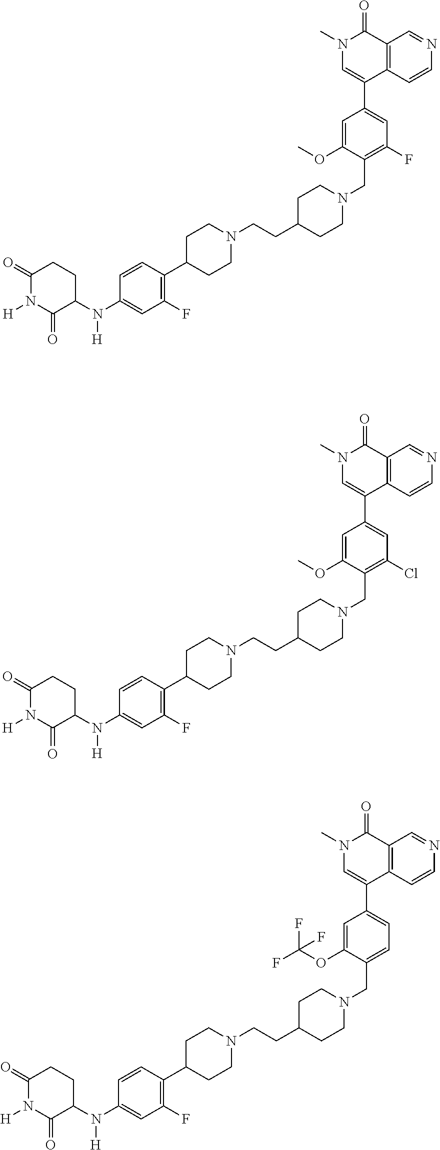 [SMILES: COc1cc(-c2cn(C)c(=O)c3cnccc23)cc(F)c1CN1CCC(CCN2CCC(c3ccc(NC4CCC(=O)NC4=O)cc3F)CC2)CC1 ; COc1cc(-c2cn(C)c(=O)c3cnccc23)cc(Cl)c1CN1CCC(CCN2CCC(c3ccc(NC4CCC(=O)NC4=O)cc3F)CC2)CC1 ; Cn1cc(-c2ccc(CN3CCC(CCN4CCC(c5ccc(NC6CCC(=O)NC6=O)cc5F)CC4)CC3)c(OC(F)(F)F)c2)c2ccncc2c1=O]

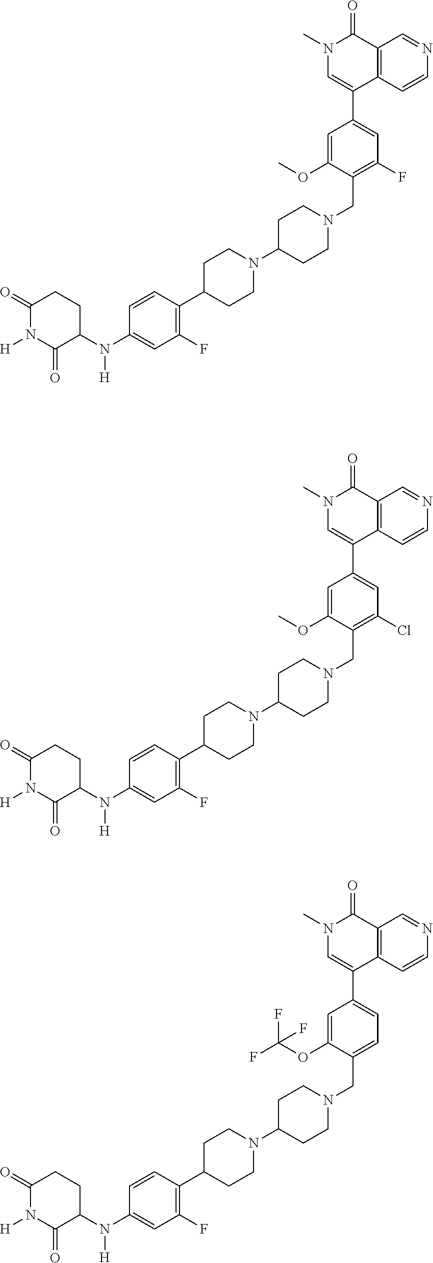 [SMILES: COc1cc(-c2cn(C)c(=O)c3cnccc23)cc(F)c1CN1CCC(N2CCC(c3ccc(NC4CCC(=O)NC4=O)cc3F)CC2)CC1 ; COc1cc(-c2cn(C)c(=O)c3cnccc23)cc(Cl)c1CN1CCC(N2CCC(c3ccc(NC4CCC(=O)NC4=O)cc3F)CC2)CC1 ; Cn1cc(-c2ccc(CN3CCC(N4CCC(c5ccc(NC6CCC(=O)NC6=O)cc5F)CC4)CC3)c(OC(F)(F)F)c2)c2ccncc2c1=O]

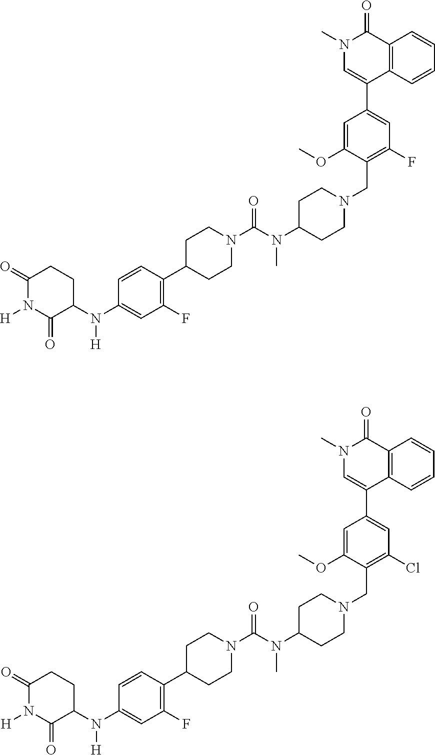 [SMILES: COc1cc(-c2cn(C)c(=O)c3ccccc23)cc(F)c1CN1CCC(N(C)C(=O)N2CCC(c3ccc(NC4CCC(=O)NC4=O)cc3F)CC2)CC1 ; COc1cc(-c2cn(C)c(=O)c3ccccc23)cc(Cl)c1CN1CCC(N(C)C(=O)N2CCC(c3ccc(NC4CCC(=O)NC4=O)cc3F)CC2)CC1]

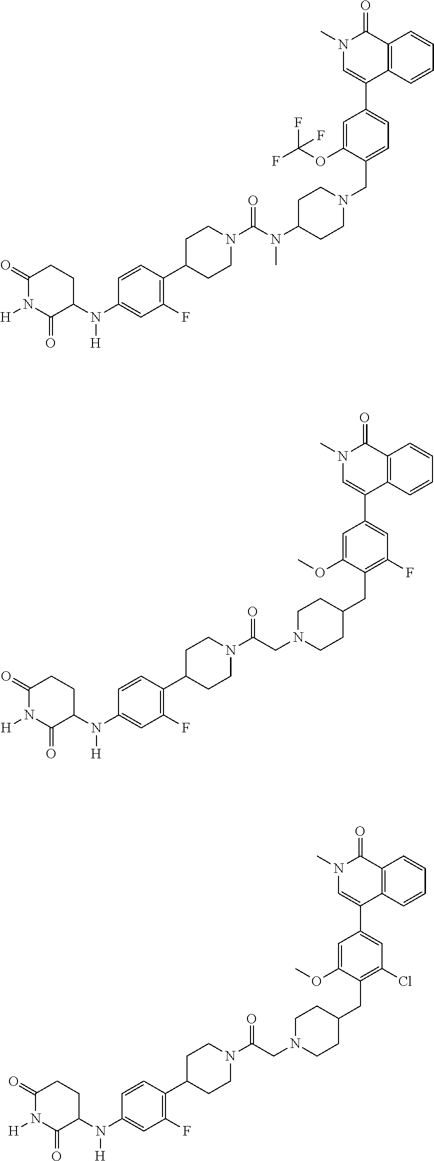 [SMILES: CN(C(=O)N1CCC(c2ccc(NC3CCC(=O)NC3=O)cc2F)CC1)C1CCN(Cc2ccc(-c3cn(C)c(=O)c4ccccc34)cc2OC(F)(F)F)CC1 ; COc1cc(-c2cn(C)c(=O)c3ccccc23)cc(F)c1CC1CCN(CC(=O)N2CCC(c3ccc(NC4CCC(=O)NC4=O)cc3F)CC2)CC1 ; COc1cc(-c2cn(C)c(=O)c3ccccc23)cc(Cl)c1CC1CCN(CC(=O)N2CCC(c3ccc(NC4CCC(=O)NC4=O)cc3F)CC2)CC1]

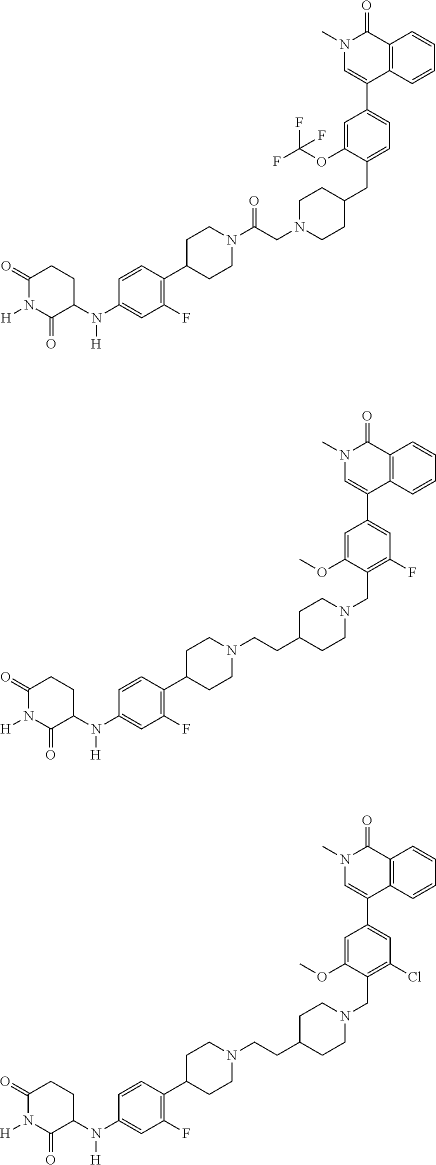 [SMILES: Cn1cc(-c2ccc(CC3CCN(CC(=O)N4CCC(c5ccc(NC6CCC(=O)NC6=O)cc5F)CC4)CC3)c(OC(F)(F)F)c2)c2ccccc2c1=O ; COc1cc(-c2cn(C)c(=O)c3ccccc23)cc(F)c1CN1CCC(CCN2CCC(c3ccc(NC4CCC(=O)NC4=O)cc3F)CC2)CC1 ; COc1cc(-c2cn(C)c(=O)c3ccccc23)cc(Cl)c1CN1CCC(CCN2CCC(c3ccc(NC4CCC(=O)NC4=O)cc3F)CC2)CC1]

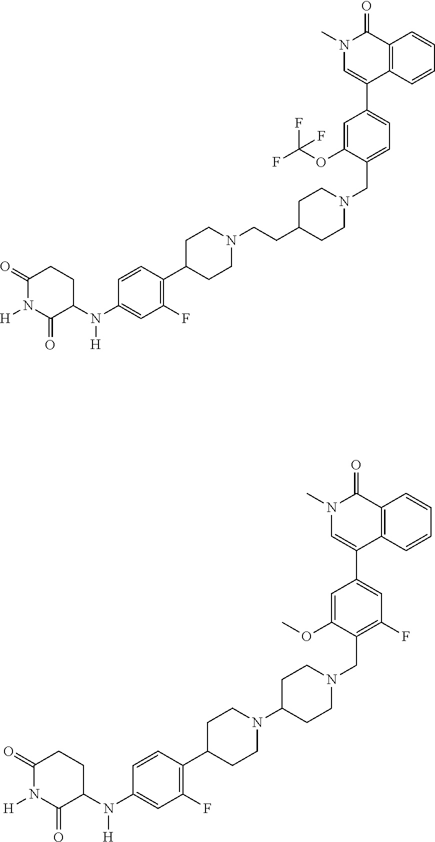 [SMILES: Cn1cc(-c2ccc(CN3CCC(CCN4CCC(c5ccc(NC6CCC(=O)NC6=O)cc5F)CC4)CC3)c(OC(F)(F)F)c2)c2ccccc2c1=O ; COc1cc(-c2cn(C)c(=O)c3ccccc23)cc(F)c1CN1CCC(N2CCC(c3ccc(NC4CCC(=O)NC4=O)cc3F)CC2)CC1]

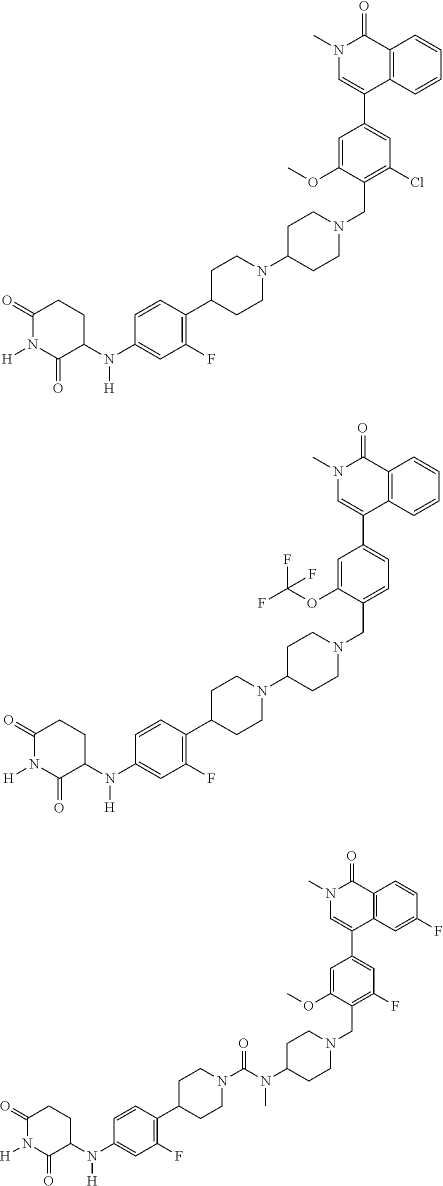 [SMILES: COc1cc(-c2cn(C)c(=O)c3ccccc23)cc(Cl)c1CN1CCC(N2CCC(c3ccc(NC4CCC(=O)NC4=O)cc3F)CC2)CC1 ; Cn1cc(-c2ccc(CN3CCC(N4CCC(c5ccc(NC6CCC(=O)NC6=O)cc5F)CC4)CC3)c(OC(F)(F)F)c2)c2ccccc2c1=O ; COc1cc(-c2cn(C)c(=O)c3ccc(F)cc23)cc(F)c1CN1CCC(N(C)C(=O)N2CCC(c3ccc(NC4CCC(=O)NC4=O)cc3F)CC2)CC1]

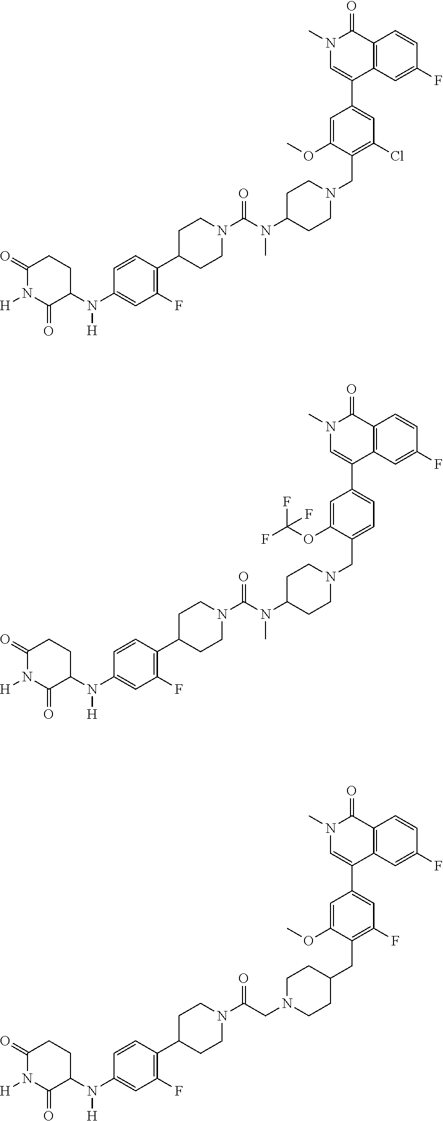 [SMILES: COc1cc(-c2cn(C)c(=O)c3ccc(F)cc23)cc(Cl)c1CN1CCC(N(C)C(=O)N2CCC(c3ccc(NC4CCC(=O)NC4=O)cc3F)CC2)CC1 ; CN(C(=O)N1CCC(c2ccc(NC3CCC(=O)NC3=O)cc2F)CC1)C1CCN(Cc2ccc(-c3cn(C)c(=O)c4ccc(F)cc34)cc2OC(F)(F)F)CC1 ; COc1cc(-c2cn(C)c(=O)c3ccc(F)cc23)cc(F)c1CC1CCN(CC(=O)N2CCC(c3ccc(NC4CCC(=O)NC4=O)cc3F)CC2)CC1]

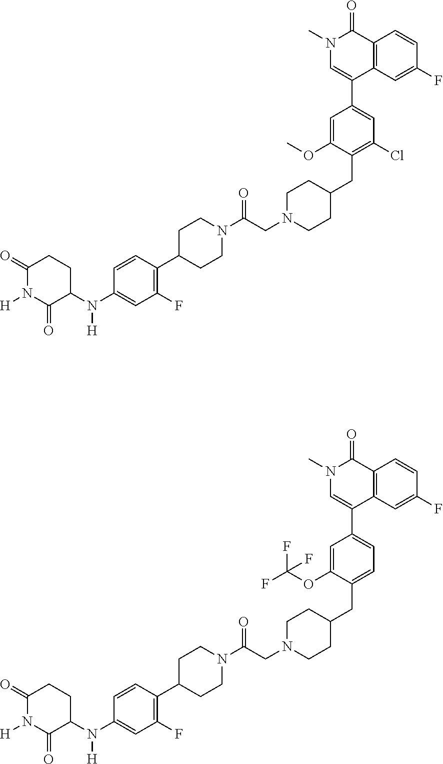 [SMILES: COc1cc(-c2cn(C)c(=O)c3ccc(F)cc23)cc(Cl)c1CC1CCN(CC(=O)N2CCC(c3ccc(NC4CCC(=O)NC4=O)cc3F)CC2)CC1 ; Cn1cc(-c2ccc(CC3CCN(CC(=O)N4CCC(c5ccc(NC6CCC(=O)NC6=O)cc5F)CC4)CC3)c(OC(F)(F)F)c2)c2cc(F)ccc2c1=O]

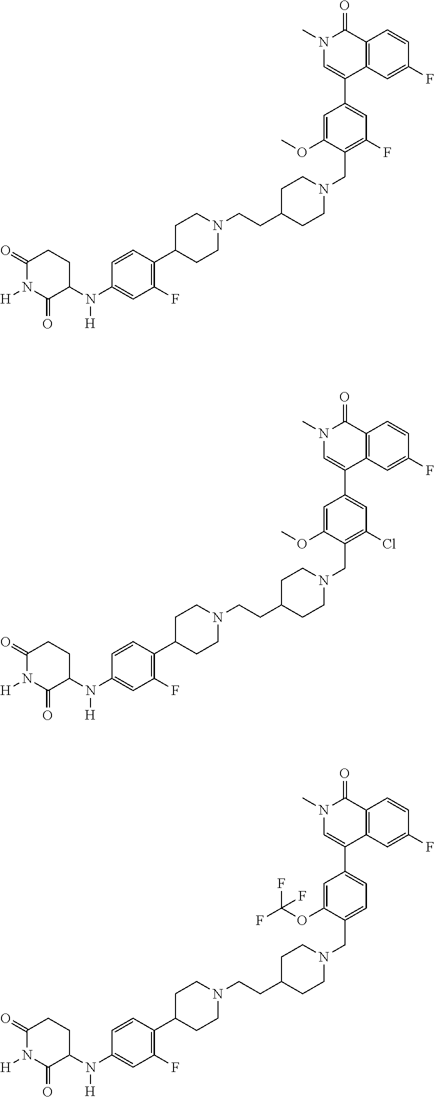 [SMILES: COc1cc(-c2cn(C)c(=O)c3ccc(F)cc23)cc(F)c1CN1CCC(CCN2CCC(c3ccc(NC4CCC(=O)NC4=O)cc3F)CC2)CC1 ; COc1cc(-c2cn(C)c(=O)c3ccc(F)cc23)cc(Cl)c1CN1CCC(CCN2CCC(c3ccc(NC4CCC(=O)NC4=O)cc3F)CC2)CC1 ; Cn1cc(-c2ccc(CN3CCC(CCN4CCC(c5ccc(NC6CCC(=O)NC6=O)cc5F)CC4)CC3)c(OC(F)(F)F)c2)c2cc(F)ccc2c1=O]

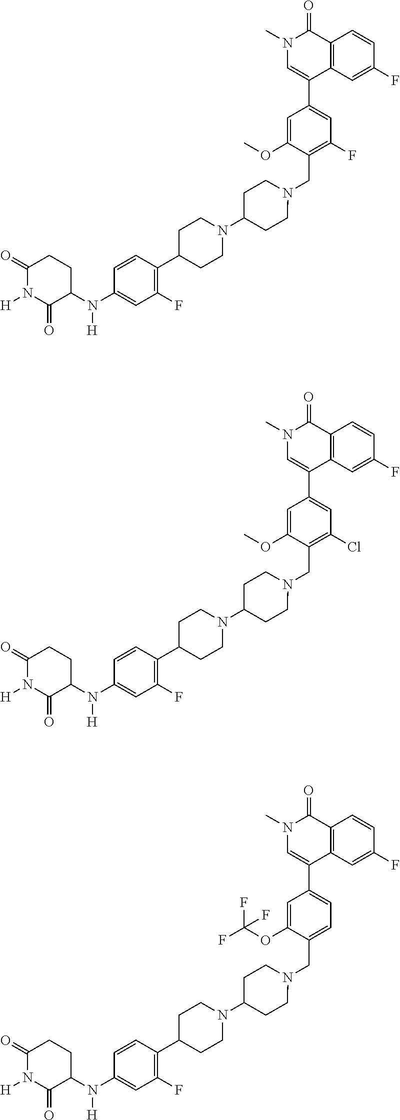 [SMILES: COc1cc(-c2cn(C)c(=O)c3ccc(F)cc23)cc(F)c1CN1CCC(N2CCC(c3ccc(NC4CCC(=O)NC4=O)cc3F)CC2)CC1 ; COc1cc(-c2cn(C)c(=O)c3ccc(F)cc23)cc(Cl)c1CN1CCC(N2CCC(c3ccc(NC4CCC(=O)NC4=O)cc3F)CC2)CC1 ; Cn1cc(-c2ccc(CN3CCC(N4CCC(c5ccc(NC6CCC(=O)NC6=O)cc5F)CC4)CC3)c(OC(F)(F)F)c2)c2cc(F)ccc2c1=O]

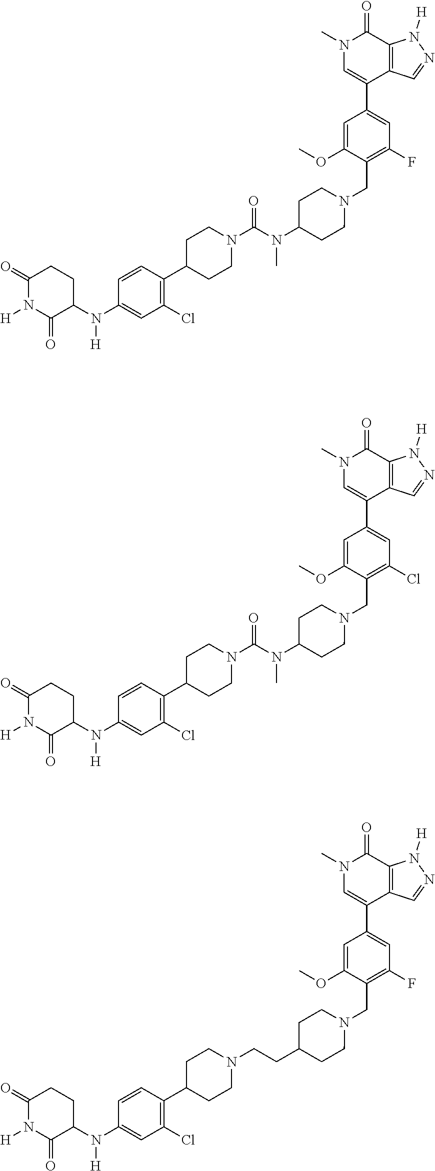 [SMILES: COc1cc(-c2cn(C)c(=O)c3[nH]ncc23)cc(F)c1CN1CCC(N(C)C(=O)N2CCC(c3ccc(NC4CCC(=O)NC4=O)cc3Cl)CC2)CC1 ; COc1cc(-c2cn(C)c(=O)c3[nH]ncc23)cc(Cl)c1CN1CCC(N(C)C(=O)N2CCC(c3ccc(NC4CCC(=O)NC4=O)cc3Cl)CC2)CC1 ; COc1cc(-c2cn(C)c(=O)c3[nH]ncc23)cc(F)c1CN1CCC(CCN2CCC(c3ccc(NC4CCC(=O)NC4=O)cc3Cl)CC2)CC1]

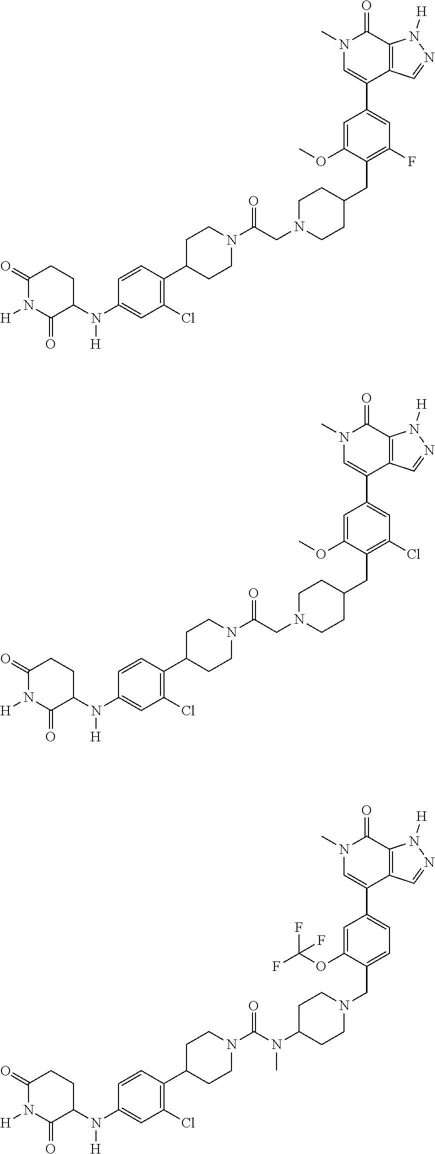 [SMILES: COc1cc(-c2cn(C)c(=O)c3[nH]ncc23)cc(F)c1CC1CCN(CC(=O)N2CCC(c3ccc(NC4CCC(=O)NC4=O)cc3Cl)CC2)CC1 ; COc1cc(-c2cn(C)c(=O)c3[nH]ncc23)cc(Cl)c1CC1CCN(CC(=O)N2CCC(c3ccc(NC4CCC(=O)NC4=O)cc3Cl)CC2)CC1 ; CN(C(=O)N1CCC(c2ccc(NC3CCC(=O)NC3=O)cc2Cl)CC1)C1CCN(Cc2ccc(-c3cn(C)c(=O)c4[nH]ncc34)cc2OC(F)(F)F)CC1]

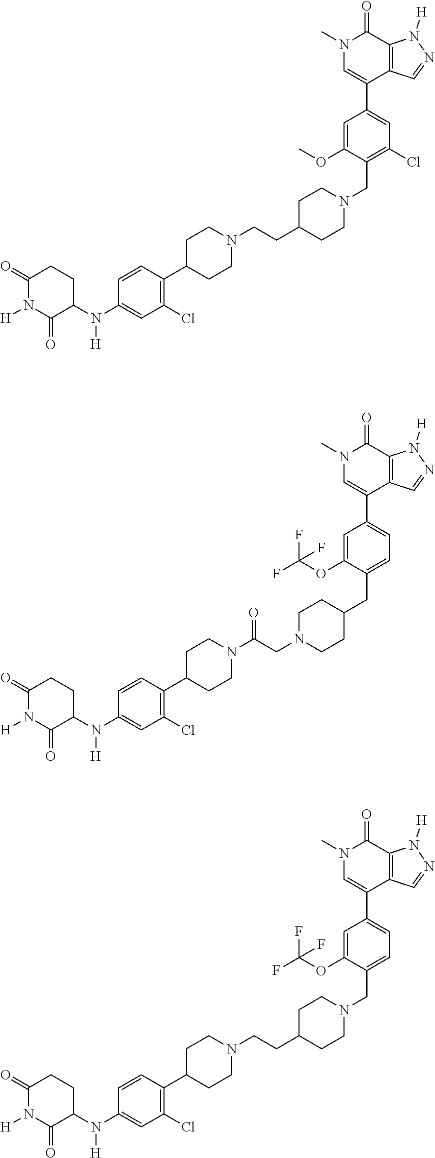 [SMILES: COc1cc(-c2cn(C)c(=O)c3[nH]ncc23)cc(Cl)c1CN1CCC(CCN2CCC(c3ccc(NC4CCC(=O)NC4=O)cc3Cl)CC2)CC1 ; Cn1cc(-c2ccc(CC3CCN(CC(=O)N4CCC(c5ccc(NC6CCC(=O)NC6=O)cc5Cl)CC4)CC3)c(OC(F)(F)F)c2)c2cn[nH]c2c1=O ; Cn1cc(-c2ccc(CN3CCC(CCN4CCC(c5ccc(NC6CCC(=O)NC6=O)cc5Cl)CC4)CC3)c(OC(F)(F)F)c2)c2cn[nH]c2c1=O]

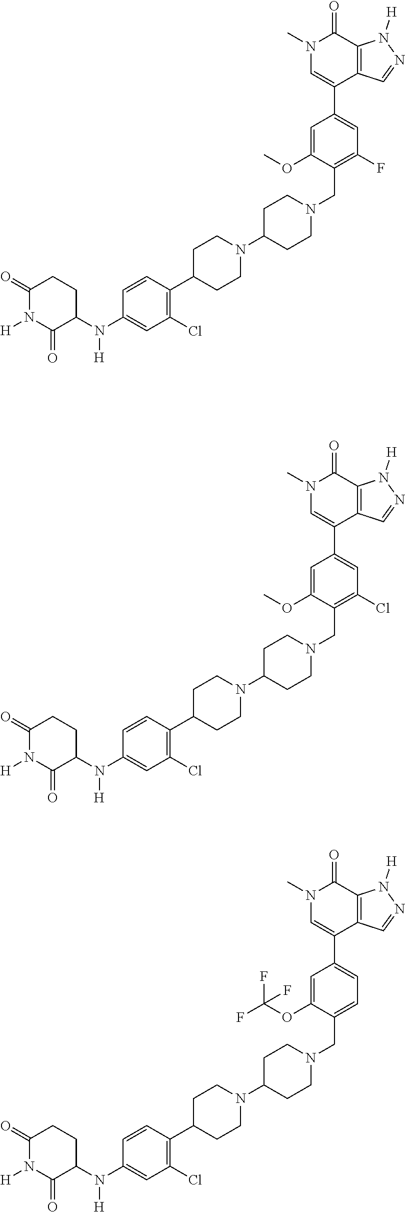 [SMILES: COc1cc(-c2cn(C)c(=O)c3[nH]ncc23)cc(F)c1CN1CCC(N2CCC(c3ccc(NC4CCC(=O)NC4=O)cc3Cl)CC2)CC1 ; COc1cc(-c2cn(C)c(=O)c3[nH]ncc23)cc(Cl)c1CN1CCC(N2CCC(c3ccc(NC4CCC(=O)NC4=O)cc3Cl)CC2)CC1 ; Cn1cc(-c2ccc(CN3CCC(N4CCC(c5ccc(NC6CCC(=O)NC6=O)cc5Cl)CC4)CC3)c(OC(F)(F)F)c2)c2cn[nH]c2c1=O]

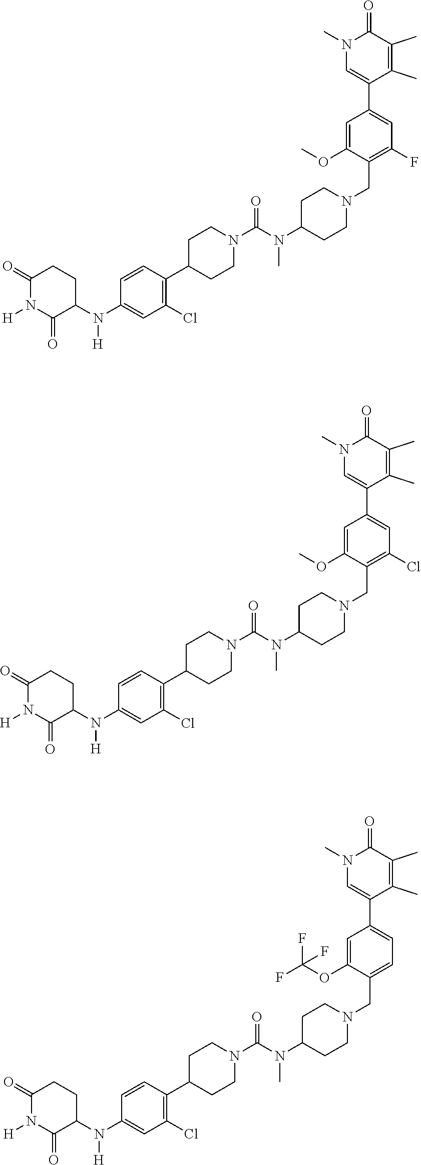 [SMILES: COc1cc(-c2cn(C)c(=O)c(C)c2C)cc(F)c1CN1CCC(N(C)C(=O)N2CCC(c3ccc(NC4CCC(=O)NC4=O)cc3Cl)CC2)CC1 ; COc1cc(-c2cn(C)c(=O)c(C)c2C)cc(Cl)c1CN1CCC(N(C)C(=O)N2CCC(c3ccc(NC4CCC(=O)NC4=O)cc3Cl)CC2)CC1 ; Cc1c(-c2ccc(CN3CCC(N(C)C(=O)N4CCC(c5ccc(NC6CCC(=O)NC6=O)cc5Cl)CC4)CC3)c(OC(F)(F)F)c2)cn(C)c(=O)c1C]

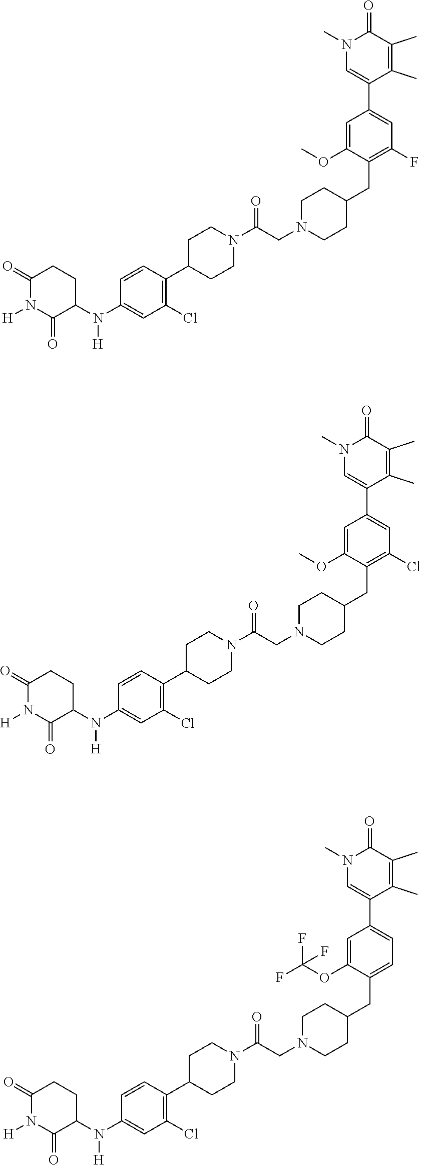 [SMILES: COc1cc(-c2cn(C)c(=O)c(C)c2C)cc(F)c1CC1CCN(CC(=O)N2CCC(c3ccc(NC4CCC(=O)NC4=O)cc3Cl)CC2)CC1 ; COc1cc(-c2cn(C)c(=O)c(C)c2C)cc(Cl)c1CC1CCN(CC(=O)N2CCC(c3ccc(NC4CCC(=O)NC4=O)cc3Cl)CC2)CC1 ; Cc1c(-c2ccc(CC3CCN(CC(=O)N4CCC(c5ccc(NC6CCC(=O)NC6=O)cc5Cl)CC4)CC3)c(OC(F)(F)F)c2)cn(C)c(=O)c1C]

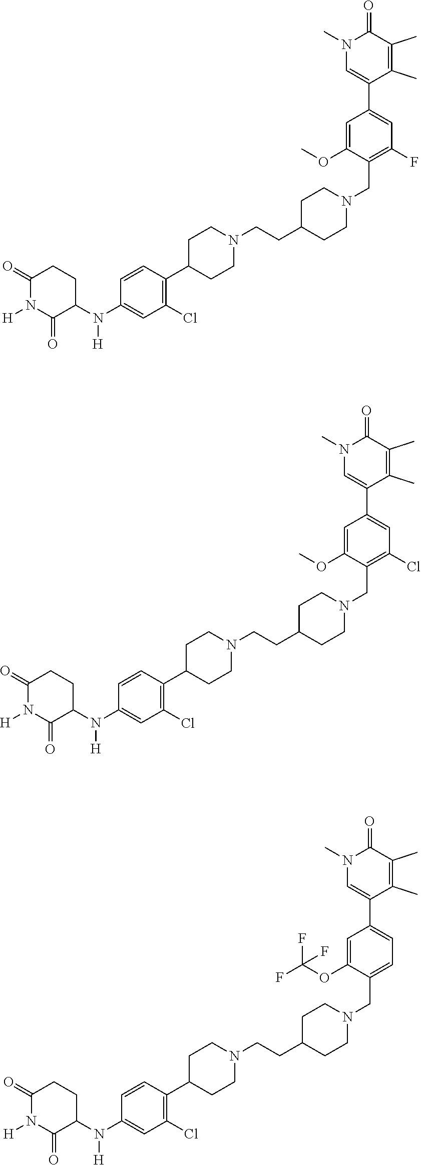 [SMILES: COc1cc(-c2cn(C)c(=O)c(C)c2C)cc(F)c1CN1CCC(CCN2CCC(c3ccc(NC4CCC(=O)NC4=O)cc3Cl)CC2)CC1 ; COc1cc(-c2cn(C)c(=O)c(C)c2C)cc(Cl)c1CN1CCC(CCN2CCC(c3ccc(NC4CCC(=O)NC4=O)cc3Cl)CC2)CC1 ; Cc1c(-c2ccc(CN3CCC(CCN4CCC(c5ccc(NC6CCC(=O)NC6=O)cc5Cl)CC4)CC3)c(OC(F)(F)F)c2)cn(C)c(=O)c1C]

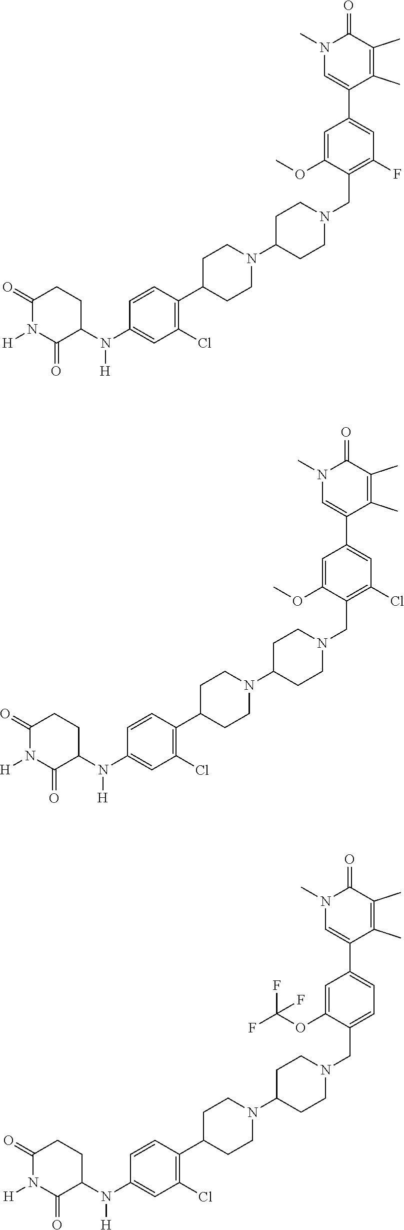 [SMILES: COc1cc(-c2cn(C)c(=O)c(C)c2C)cc(F)c1CN1CCC(N2CCC(c3ccc(NC4CCC(=O)NC4=O)cc3Cl)CC2)CC1 ; COc1cc(-c2cn(C)c(=O)c(C)c2C)cc(Cl)c1CN1CCC(N2CCC(c3ccc(NC4CCC(=O)NC4=O)cc3Cl)CC2)CC1 ; Cc1c(-c2ccc(CN3CCC(N4CCC(c5ccc(NC6CCC(=O)NC6=O)cc5Cl)CC4)CC3)c(OC(F)(F)F)c2)cn(C)c(=O)c1C]

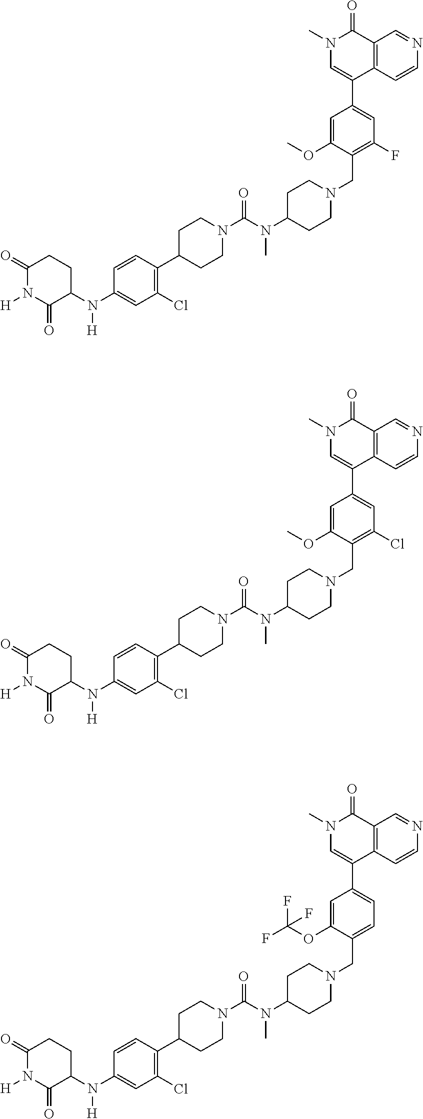 [SMILES: COc1cc(-c2cn(C)c(=O)c3cnccc23)cc(F)c1CN1CCC(N(C)C(=O)N2CCC(c3ccc(NC4CCC(=O)NC4=O)cc3Cl)CC2)CC1 ; COc1cc(-c2cn(C)c(=O)c3cnccc23)cc(Cl)c1CN1CCC(N(C)C(=O)N2CCC(c3ccc(NC4CCC(=O)NC4=O)cc3Cl)CC2)CC1 ; CN(C(=O)N1CCC(c2ccc(NC3CCC(=O)NC3=O)cc2Cl)CC1)C1CCN(Cc2ccc(-c3cn(C)c(=O)c4cnccc34)cc2OC(F)(F)F)CC1]

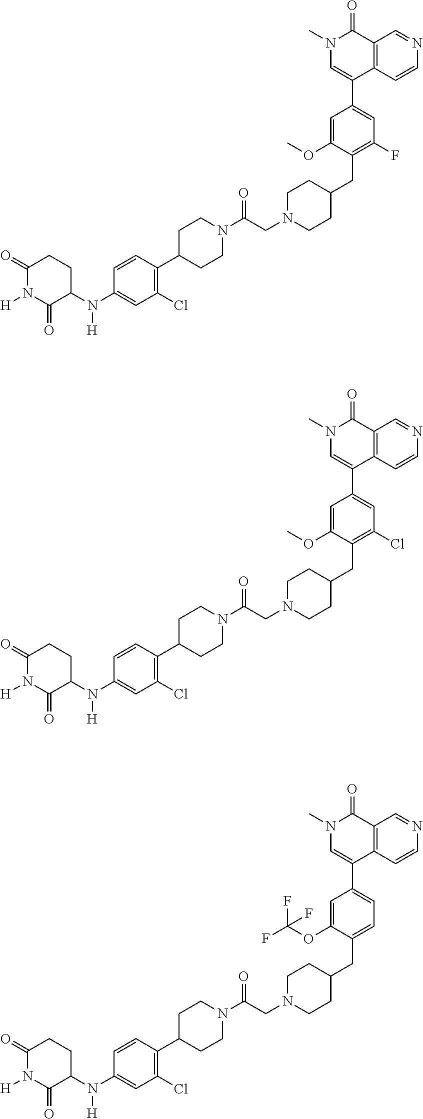 [SMILES: COc1cc(-c2cn(C)c(=O)c3cnccc23)cc(F)c1CC1CCN(CC(=O)N2CCC(c3ccc(NC4CCC(=O)NC4=O)cc3Cl)CC2)CC1 ; COc1cc(-c2cn(C)c(=O)c3cnccc23)cc(Cl)c1CC1CCN(CC(=O)N2CCC(c3ccc(NC4CCC(=O)NC4=O)cc3Cl)CC2)CC1 ; Cn1cc(-c2ccc(CC3CCN(CC(=O)N4CCC(c5ccc(NC6CCC(=O)NC6=O)cc5Cl)CC4)CC3)c(OC(F)(F)F)c2)c2ccncc2c1=O]

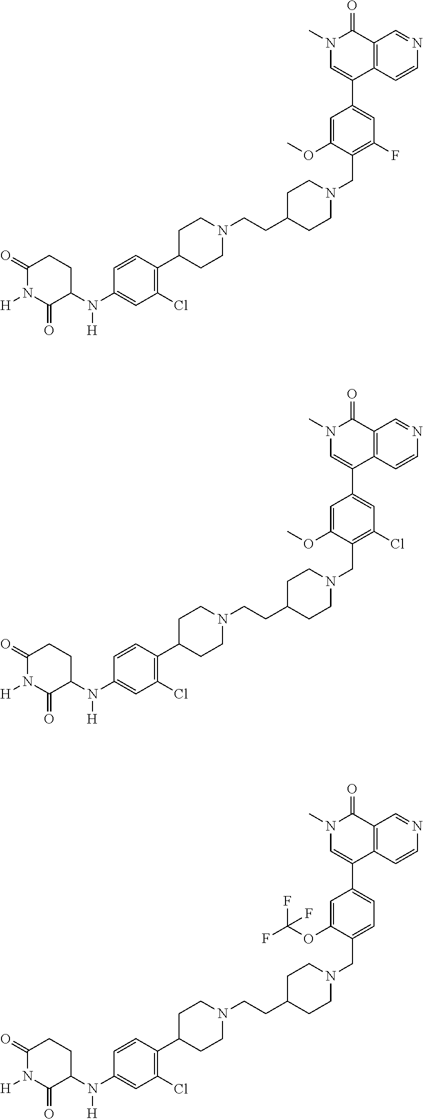 [SMILES: COc1cc(-c2cn(C)c(=O)c3cnccc23)cc(F)c1CN1CCC(CCN2CCC(c3ccc(NC4CCC(=O)NC4=O)cc3Cl)CC2)CC1 ; COc1cc(-c2cn(C)c(=O)c3cnccc23)cc(Cl)c1CN1CCC(CCN2CCC(c3ccc(NC4CCC(=O)NC4=O)cc3Cl)CC2)CC1 ; Cn1cc(-c2ccc(CN3CCC(CCN4CCC(c5ccc(NC6CCC(=O)NC6=O)cc5Cl)CC4)CC3)c(OC(F)(F)F)c2)c2ccncc2c1=O]

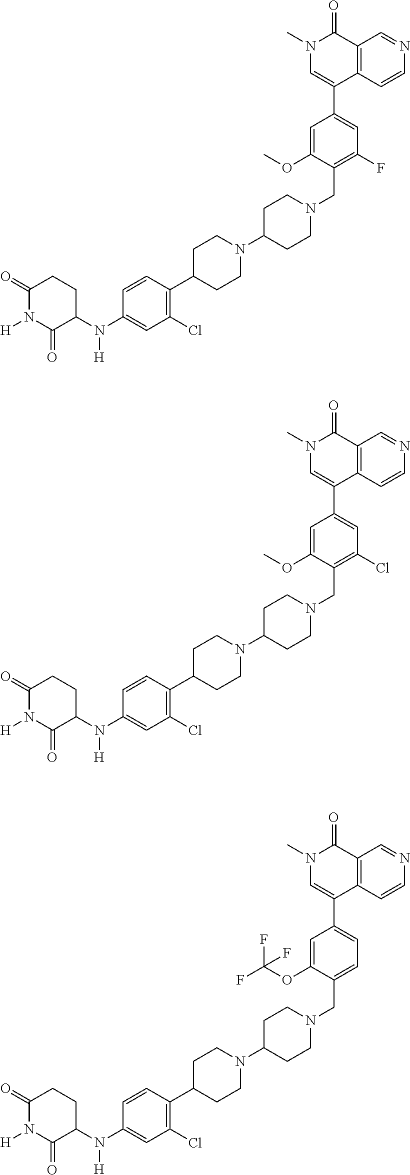 [SMILES: COc1cc(-c2cn(C)c(=O)c3cnccc23)cc(F)c1CN1CCC(N2CCC(c3ccc(NC4CCC(=O)NC4=O)cc3Cl)CC2)CC1 ; COc1cc(-c2cn(C)c(=O)c3cnccc23)cc(Cl)c1CN1CCC(N2CCC(c3ccc(NC4CCC(=O)NC4=O)cc3Cl)CC2)CC1 ; Cn1cc(-c2ccc(CN3CCC(N4CCC(c5ccc(NC6CCC(=O)NC6=O)cc5Cl)CC4)CC3)c(OC(F)(F)F)c2)c2ccncc2c1=O]

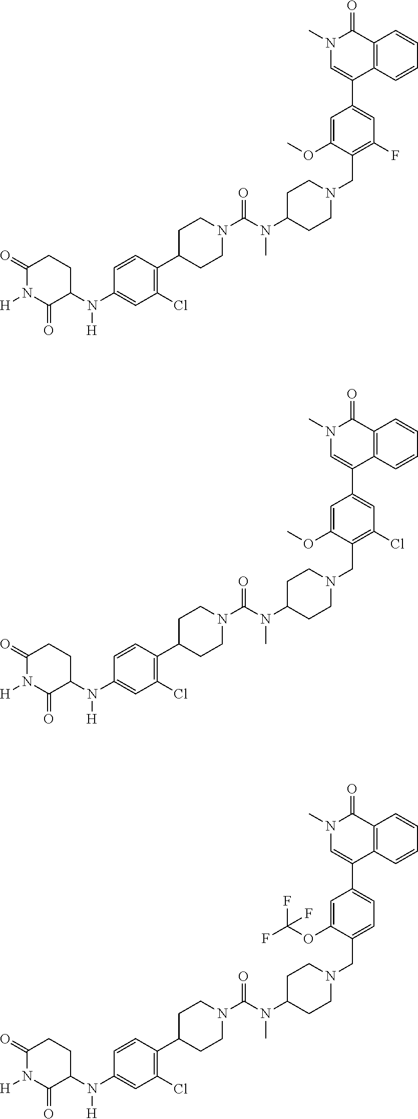 [SMILES: COc1cc(-c2cn(C)c(=O)c3ccccc23)cc(F)c1CN1CCC(N(C)C(=O)N2CCC(c3ccc(NC4CCC(=O)NC4=O)cc3Cl)CC2)CC1 ; COc1cc(-c2cn(C)c(=O)c3ccccc23)cc(Cl)c1CN1CCC(N(C)C(=O)N2CCC(c3ccc(NC4CCC(=O)NC4=O)cc3Cl)CC2)CC1 ; CN(C(=O)N1CCC(c2ccc(NC3CCC(=O)NC3=O)cc2Cl)CC1)C1CCN(Cc2ccc(-c3cn(C)c(=O)c4ccccc34)cc2OC(F)(F)F)CC1]

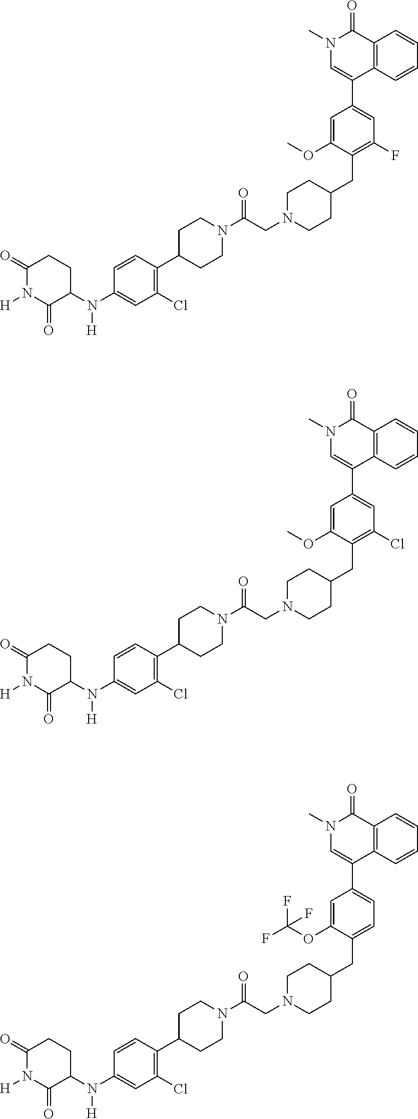 [SMILES: COc1cc(-c2cn(C)c(=O)c3ccccc23)cc(F)c1CC1CCN(CC(=O)N2CCC(c3ccc(NC4CCC(=O)NC4=O)cc3Cl)CC2)CC1 ; COc1cc(-c2cn(C)c(=O)c3ccccc23)cc(Cl)c1CC1CCN(CC(=O)N2CCC(c3ccc(NC4CCC(=O)NC4=O)cc3Cl)CC2)CC1 ; Cn1cc(-c2ccc(CC3CCN(CC(=O)N4CCC(c5ccc(NC6CCC(=O)NC6=O)cc5Cl)CC4)CC3)c(OC(F)(F)F)c2)c2ccccc2c1=O]

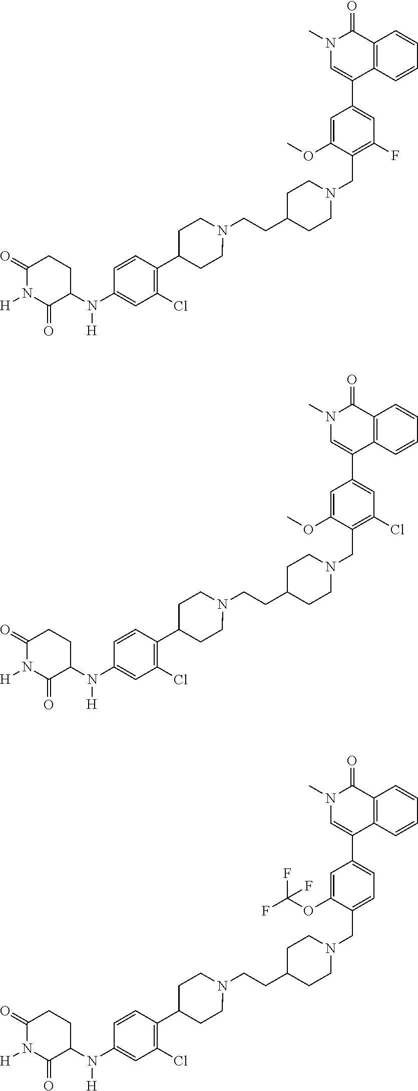 [SMILES: COc1cc(-c2cn(C)c(=O)c3ccccc23)cc(F)c1CN1CCC(CCN2CCC(c3ccc(NC4CCC(=O)NC4=O)cc3Cl)CC2)CC1 ; COc1cc(-c2cn(C)c(=O)c3ccccc23)cc(Cl)c1CN1CCC(CCN2CCC(c3ccc(NC4CCC(=O)NC4=O)cc3Cl)CC2)CC1 ; Cn1cc(-c2ccc(CN3CCC(CCN4CCC(c5ccc(NC6CCC(=O)NC6=O)cc5Cl)CC4)CC3)c(OC(F)(F)F)c2)c2ccccc2c1=O]

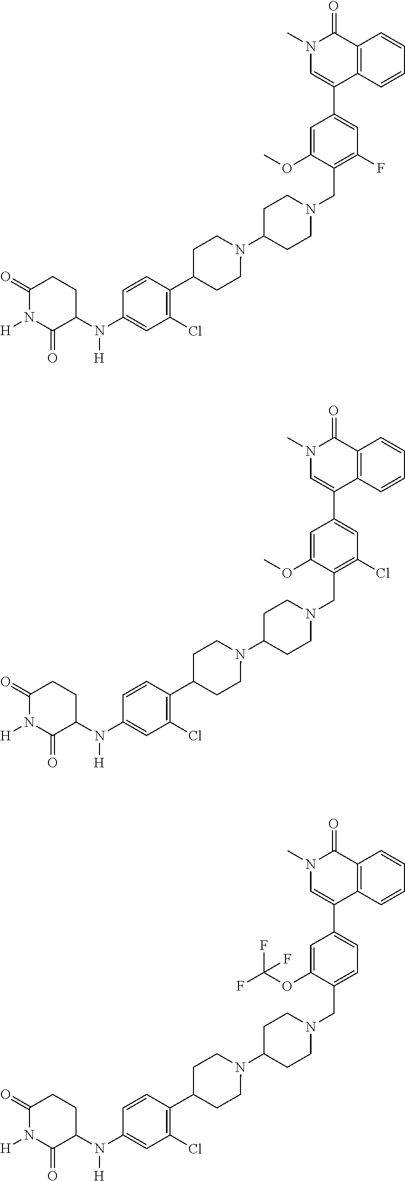 [SMILES: COc1cc(-c2cn(C)c(=O)c3ccccc23)cc(F)c1CN1CCC(N2CCC(c3ccc(NC4CCC(=O)NC4=O)cc3Cl)CC2)CC1 ; COc1cc(-c2cn(C)c(=O)c3ccccc23)cc(Cl)c1CN1CCC(N2CCC(c3ccc(NC4CCC(=O)NC4=O)cc3Cl)CC2)CC1 ; Cn1cc(-c2ccc(CN3CCC(N4CCC(c5ccc(NC6CCC(=O)NC6=O)cc5Cl)CC4)CC3)c(OC(F)(F)F)c2)c2ccccc2c1=O]

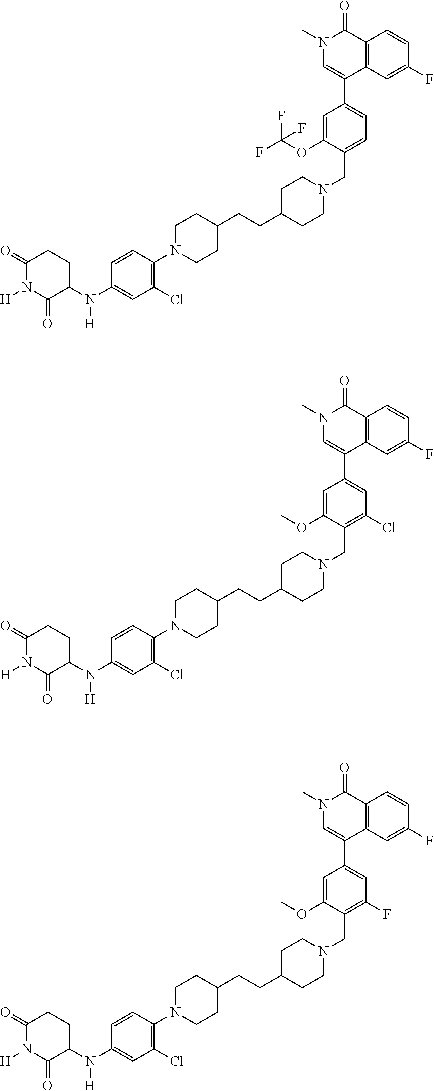 [SMILES: Cn1cc(-c2ccc(CN3CCC(CCC4CCN(c5ccc(NC6CCC(=O)NC6=O)cc5Cl)CC4)CC3)c(OC(F)(F)F)c2)c2cc(F)ccc2c1=O ; COc1cc(-c2cn(C)c(=O)c3ccc(F)cc23)cc(Cl)c1CN1CCC(CCC2CCN(c3ccc(NC4CCC(=O)NC4=O)cc3Cl)CC2)CC1 ; COc1cc(-c2cn(C)c(=O)c3ccc(F)cc23)cc(F)c1CN1CCC(CCC2CCN(c3ccc(NC4CCC(=O)NC4=O)cc3Cl)CC2)CC1]

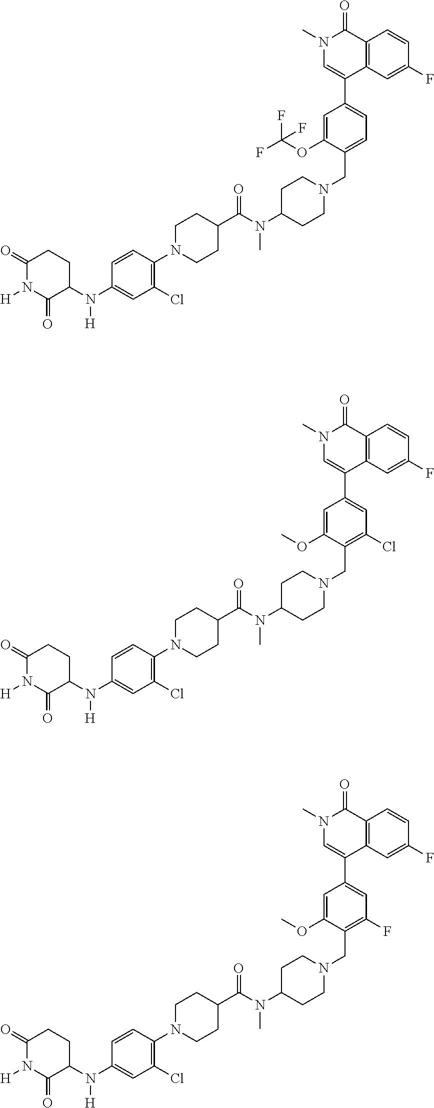 [SMILES: CN(C(=O)C1CCN(c2ccc(NC3CCC(=O)NC3=O)cc2Cl)CC1)C1CCN(Cc2ccc(-c3cn(C)c(=O)c4ccc(F)cc34)cc2OC(F)(F)F)CC1 ; COc1cc(-c2cn(C)c(=O)c3ccc(F)cc23)cc(Cl)c1CN1CCC(N(C)C(=O)C2CCN(c3ccc(NC4CCC(=O)NC4=O)cc3Cl)CC2)CC1 ; COc1cc(-c2cn(C)c(=O)c3ccc(F)cc23)cc(F)c1CN1CCC(N(C)C(=O)C2CCN(c3ccc(NC4CCC(=O)NC4=O)cc3Cl)CC2)CC1]

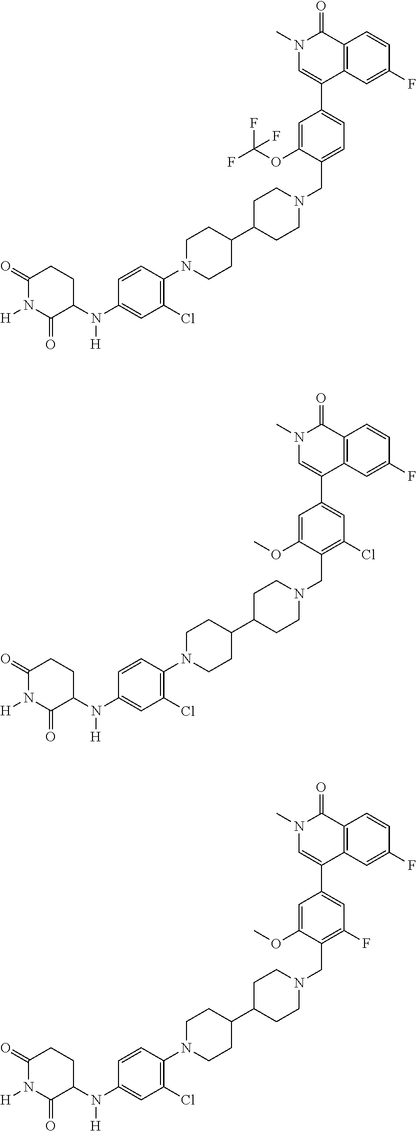 [SMILES: Cn1cc(-c2ccc(CN3CCC(C4CCN(c5ccc(NC6CCC(=O)NC6=O)cc5Cl)CC4)CC3)c(OC(F)(F)F)c2)c2cc(F)ccc2c1=O ; COc1cc(-c2cn(C)c(=O)c3ccc(F)cc23)cc(Cl)c1CN1CCC(C2CCN(c3ccc(NC4CCC(=O)NC4=O)cc3Cl)CC2)CC1 ; COc1cc(-c2cn(C)c(=O)c3ccc(F)cc23)cc(F)c1CN1CCC(C2CCN(c3ccc(NC4CCC(=O)NC4=O)cc3Cl)CC2)CC1]

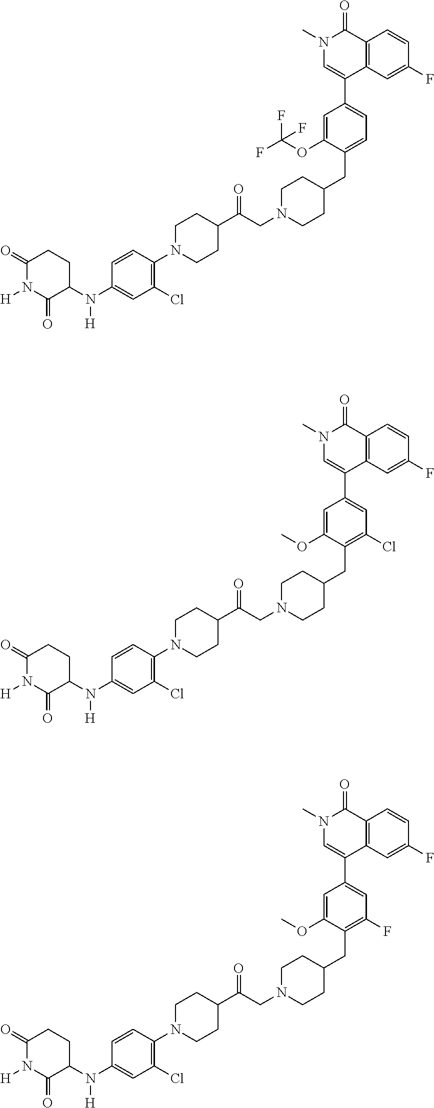 [SMILES: Cn1cc(-c2ccc(CC3CCN(CC(=O)C4CCN(c5ccc(NC6CCC(=O)NC6=O)cc5Cl)CC4)CC3)c(OC(F)(F)F)c2)c2cc(F)ccc2c1=O ; COc1cc(-c2cn(C)c(=O)c3ccc(F)cc23)cc(Cl)c1CC1CCN(CC(=O)C2CCN(c3ccc(NC4CCC(=O)NC4=O)cc3Cl)CC2)CC1 ; COc1cc(-c2cn(C)c(=O)c3ccc(F)cc23)cc(F)c1CC1CCN(CC(=O)C2CCN(c3ccc(NC4CCC(=O)NC4=O)cc3Cl)CC2)CC1]

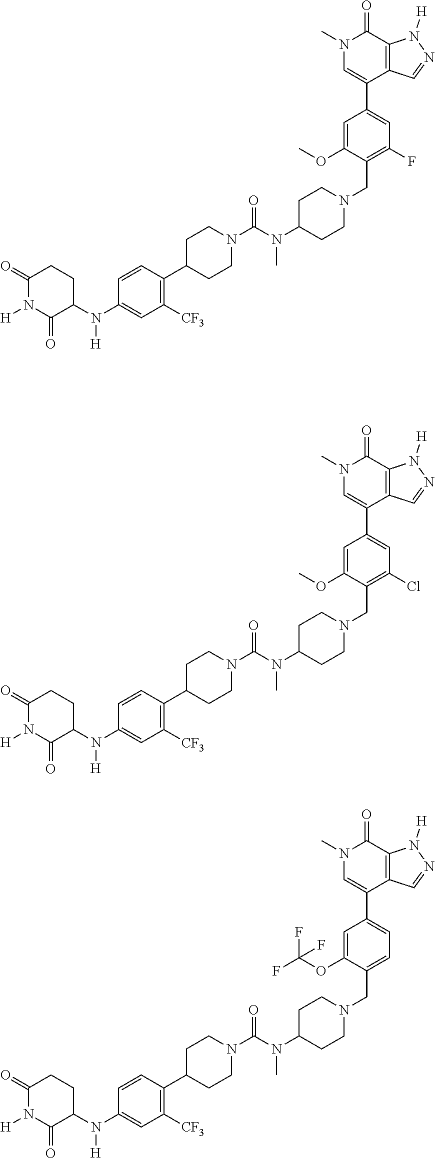 [SMILES: COc1cc(-c2cn(C)c(=O)c3[nH]ncc23)cc(F)c1CN1CCC(N(C)C(=O)N2CCC(c3ccc(NC4CCC(=O)NC4=O)cc3C(F)(F)F)CC2)CC1 ; COc1cc(-c2cn(C)c(=O)c3[nH]ncc23)cc(Cl)c1CN1CCC(N(C)C(=O)N2CCC(c3ccc(NC4CCC(=O)NC4=O)cc3C(F)(F)F)CC2)CC1 ; CN(C(=O)N1CCC(c2ccc(NC3CCC(=O)NC3=O)cc2C(F)(F)F)CC1)C1CCN(Cc2ccc(-c3cn(C)c(=O)c4[nH]ncc34)cc2OC(F)(F)F)CC1]

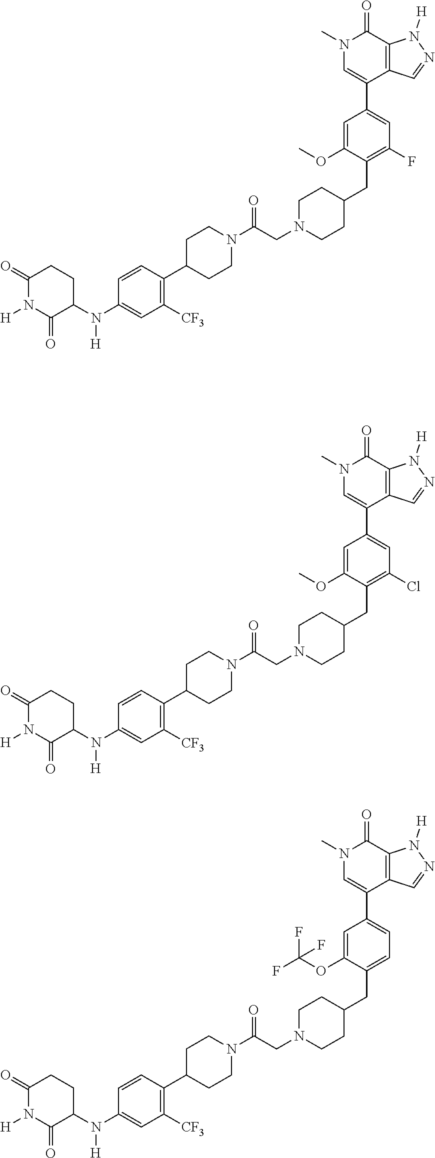 [SMILES: COc1cc(-c2cn(C)c(=O)c3[nH]ncc23)cc(F)c1CC1CCN(CC(=O)N2CCC(c3ccc(NC4CCC(=O)NC4=O)cc3C(F)(F)F)CC2)CC1 ; COc1cc(-c2cn(C)c(=O)c3[nH]ncc23)cc(Cl)c1CC1CCN(CC(=O)N2CCC(c3ccc(NC4CCC(=O)NC4=O)cc3C(F)(F)F)CC2)CC1 ; Cn1cc(-c2ccc(CC3CCN(CC(=O)N4CCC(c5ccc(NC6CCC(=O)NC6=O)cc5C(F)(F)F)CC4)CC3)c(OC(F)(F)F)c2)c2cn[nH]c2c1=O]

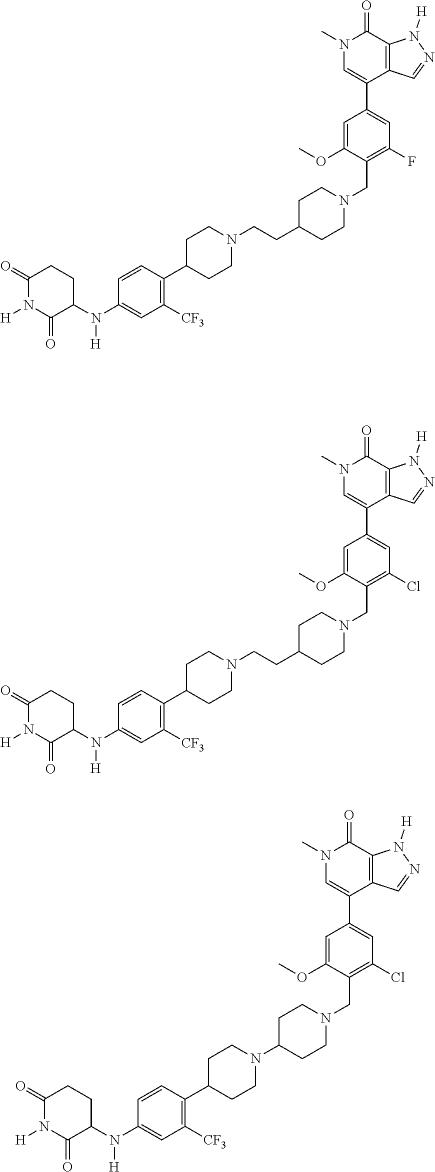 [SMILES: COc1cc(-c2cn(C)c(=O)c3[nH]ncc23)cc(F)c1CN1CCC(CCN2CCC(c3ccc(NC4CCC(=O)NC4=O)cc3C(F)(F)F)CC2)CC1 ; COc1cc(-c2cn(C)c(=O)c3[nH]ncc23)cc(Cl)c1CN1CCC(CCN2CCC(c3ccc(NC4CCC(=O)NC4=O)cc3C(F)(F)F)CC2)CC1 ; COc1cc(-c2cn(C)c(=O)c3[nH]ncc23)cc(Cl)c1CN1CCC(N2CCC(c3ccc(NC4CCC(=O)NC4=O)cc3C(F)(F)F)CC2)CC1]

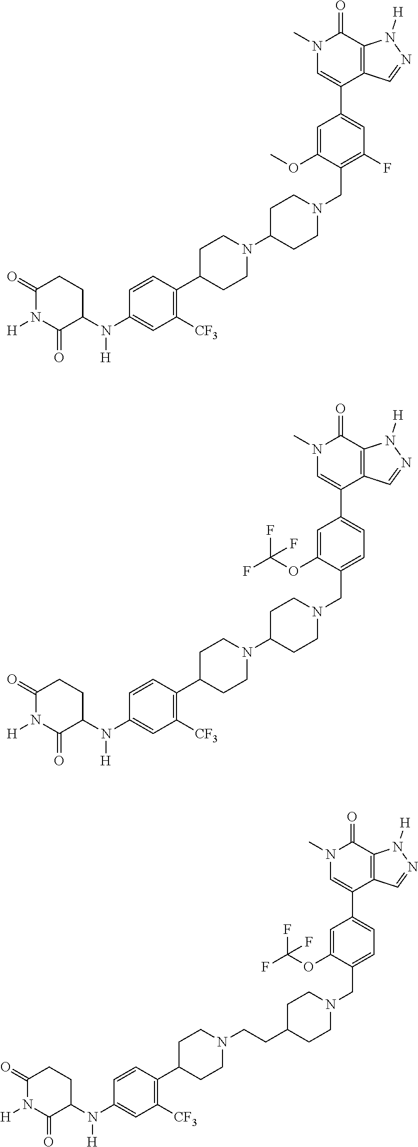 [SMILES: COc1cc(-c2cn(C)c(=O)c3[nH]ncc23)cc(F)c1CN1CCC(N2CCC(c3ccc(NC4CCC(=O)NC4=O)cc3C(F)(F)F)CC2)CC1 ; Cn1cc(-c2ccc(CN3CCC(N4CCC(c5ccc(NC6CCC(=O)NC6=O)cc5C(F)(F)F)CC4)CC3)c(OC(F)(F)F)c2)c2cn[nH]c2c1=O ; Cn1cc(-c2ccc(CN3CCC(CCN4CCC(c5ccc(NC6CCC(=O)NC6=O)cc5C(F)(F)F)CC4)CC3)c(OC(F)(F)F)c2)c2cn[nH]c2c1=O]

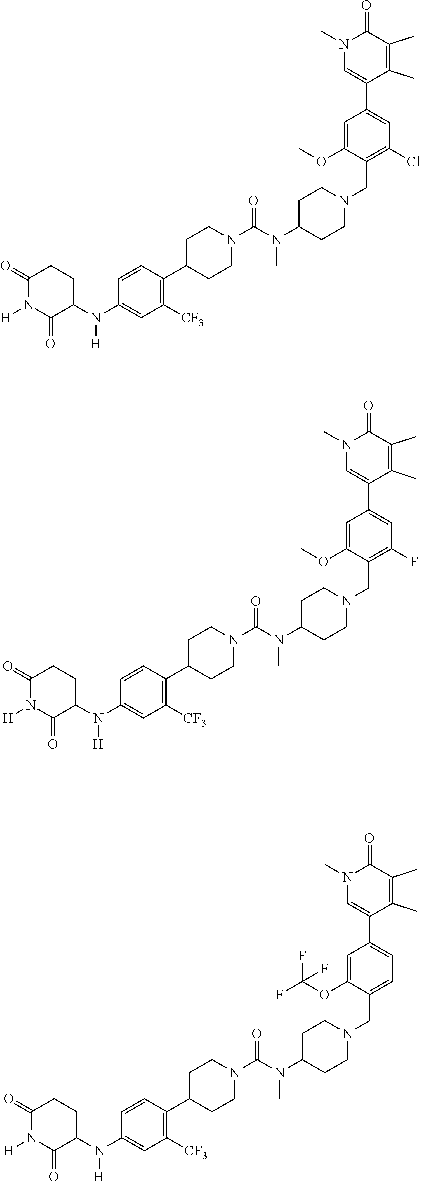 [SMILES: COc1cc(-c2cn(C)c(=O)c(C)c2C)cc(Cl)c1CN1CCC(N(C)C(=O)N2CCC(c3ccc(NC4CCC(=O)NC4=O)cc3C(F)(F)F)CC2)CC1 ; COc1cc(-c2cn(C)c(=O)c(C)c2C)cc(F)c1CN1CCC(N(C)C(=O)N2CCC(c3ccc(NC4CCC(=O)NC4=O)cc3C(F)(F)F)CC2)CC1 ; Cc1c(-c2ccc(CN3CCC(N(C)C(=O)N4CCC(c5ccc(NC6CCC(=O)NC6=O)cc5C(F)(F)F)CC4)CC3)c(OC(F)(F)F)c2)cn(C)c(=O)c1C]

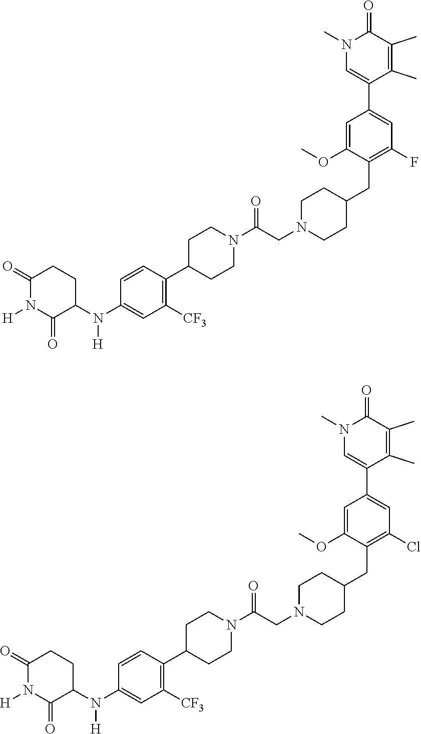 [SMILES: COc1cc(-c2cn(C)c(=O)c(C)c2C)cc(F)c1CC1CCN(CC(=O)N2CCC(c3ccc(NC4CCC(=O)NC4=O)cc3C(F)(F)F)CC2)CC1 ; COc1cc(-c2cn(C)c(=O)c(C)c2C)cc(Cl)c1CC1CCN(CC(=O)N2CCC(c3ccc(NC4CCC(=O)NC4=O)cc3C(F)(F)F)CC2)CC1]

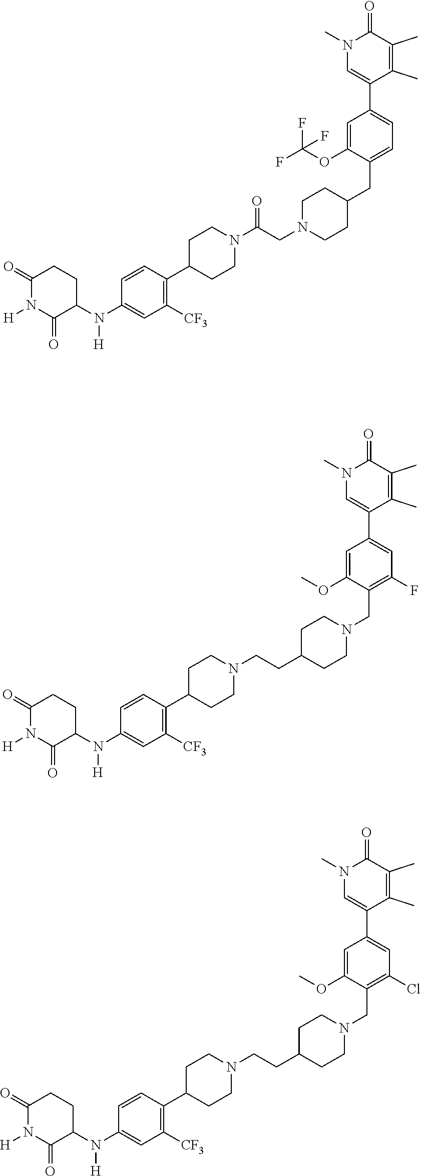 [SMILES: Cc1c(-c2ccc(CC3CCN(CC(=O)N4CCC(c5ccc(NC6CCC(=O)NC6=O)cc5C(F)(F)F)CC4)CC3)c(OC(F)(F)F)c2)cn(C)c(=O)c1C ; COc1cc(-c2cn(C)c(=O)c(C)c2C)cc(F)c1CN1CCC(CCN2CCC(c3ccc(NC4CCC(=O)NC4=O)cc3C(F)(F)F)CC2)CC1 ; COc1cc(-c2cn(C)c(=O)c(C)c2C)cc(Cl)c1CN1CCC(CCN2CCC(c3ccc(NC4CCC(=O)NC4=O)cc3C(F)(F)F)CC2)CC1]

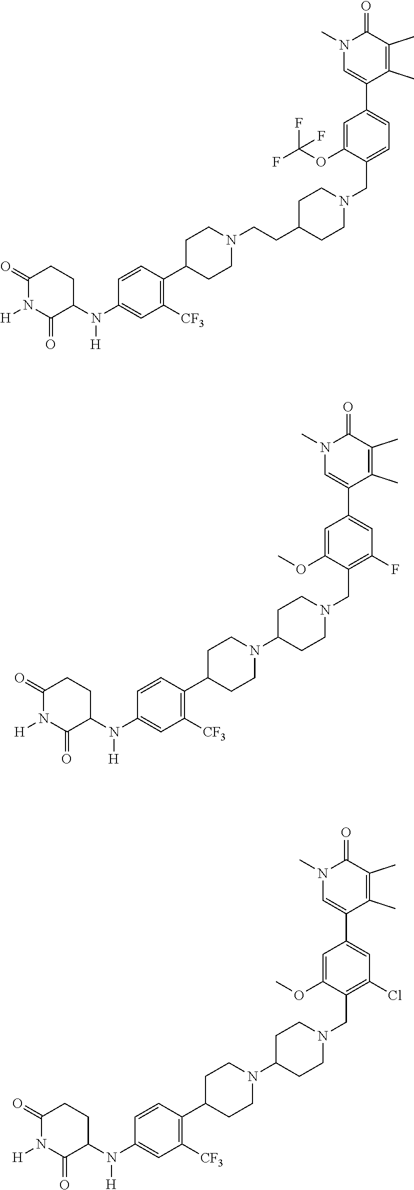 [SMILES: Cc1c(-c2ccc(CN3CCC(CCN4CCC(c5ccc(NC6CCC(=O)NC6=O)cc5C(F)(F)F)CC4)CC3)c(OC(F)(F)F)c2)cn(C)c(=O)c1C ; COc1cc(-c2cn(C)c(=O)c(C)c2C)cc(F)c1CN1CCC(N2CCC(c3ccc(NC4CCC(=O)NC4=O)cc3C(F)(F)F)CC2)CC1 ; COc1cc(-c2cn(C)c(=O)c(C)c2C)cc(Cl)c1CN1CCC(N2CCC(c3ccc(NC4CCC(=O)NC4=O)cc3C(F)(F)F)CC2)CC1]

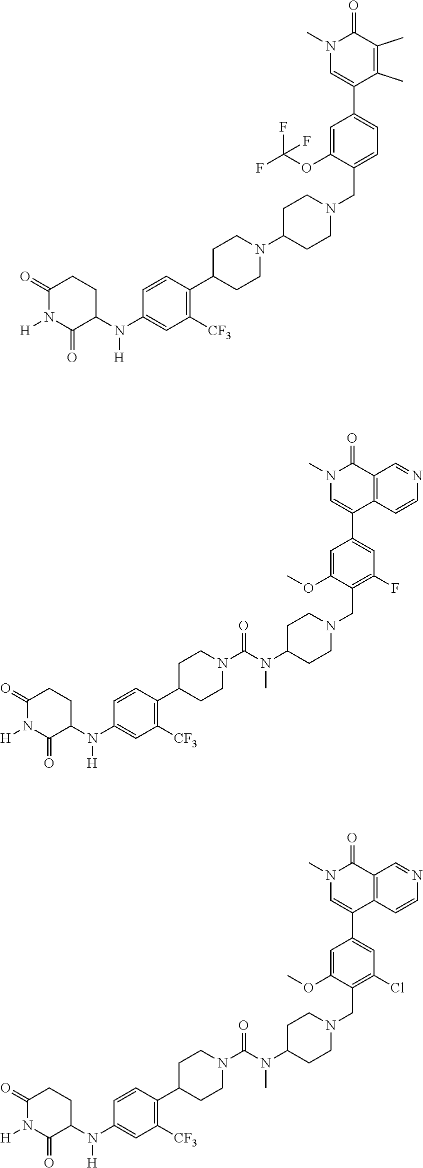 [SMILES: Cc1c(-c2ccc(CN3CCC(N4CCC(c5ccc(NC6CCC(=O)NC6=O)cc5C(F)(F)F)CC4)CC3)c(OC(F)(F)F)c2)cn(C)c(=O)c1C ; COc1cc(-c2cn(C)c(=O)c3cnccc23)cc(F)c1CN1CCC(N(C)C(=O)N2CCC(c3ccc(NC4CCC(=O)NC4=O)cc3C(F)(F)F)CC2)CC1 ; COc1cc(-c2cn(C)c(=O)c3cnccc23)cc(Cl)c1CN1CCC(N(C)C(=O)N2CCC(c3ccc(NC4CCC(=O)NC4=O)cc3C(F)(F)F)CC2)CC1]

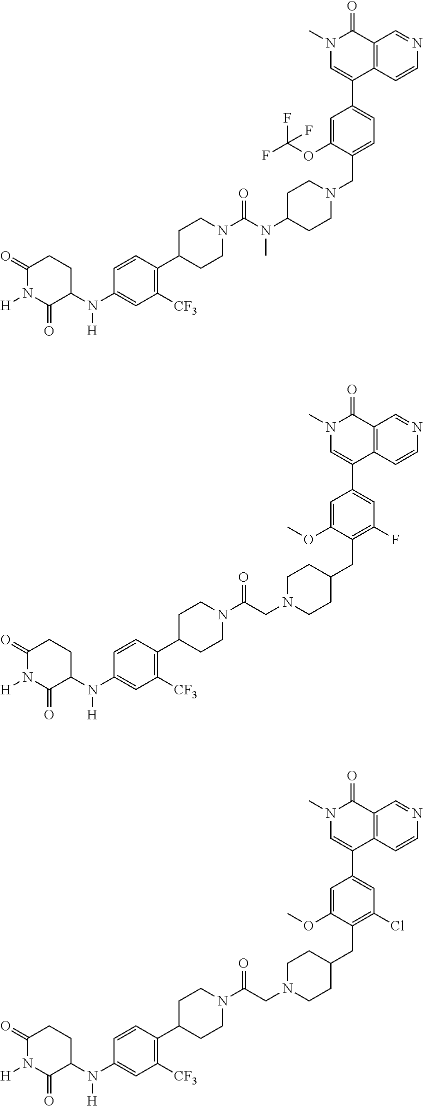 [SMILES: CN(C(=O)N1CCC(c2ccc(NC3CCC(=O)NC3=O)cc2C(F)(F)F)CC1)C1CCN(Cc2ccc(-c3cn(C)c(=O)c4cnccc34)cc2OC(F)(F)F)CC1 ; COc1cc(-c2cn(C)c(=O)c3cnccc23)cc(F)c1CC1CCN(CC(=O)N2CCC(c3ccc(NC4CCC(=O)NC4=O)cc3C(F)(F)F)CC2)CC1 ; COc1cc(-c2cn(C)c(=O)c3cnccc23)cc(Cl)c1CC1CCN(CC(=O)N2CCC(c3ccc(NC4CCC(=O)NC4=O)cc3C(F)(F)F)CC2)CC1]

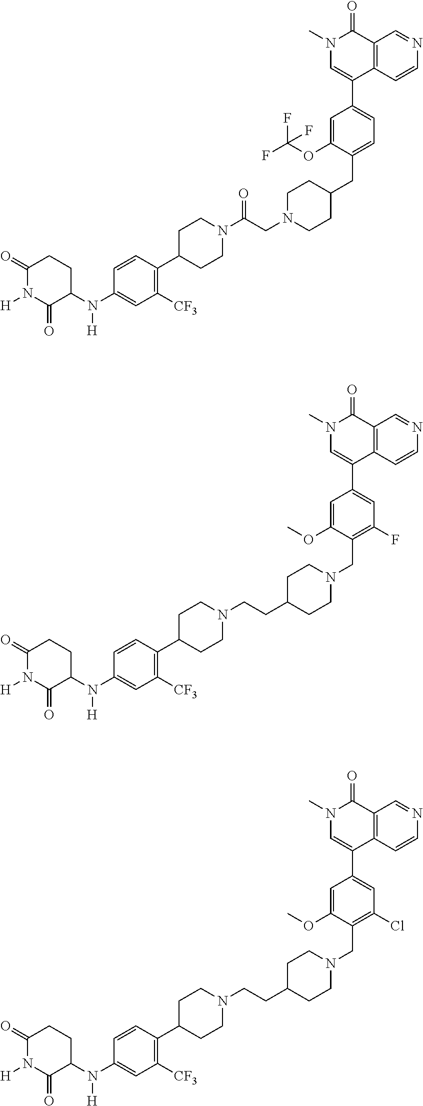 [SMILES: Cn1cc(-c2ccc(CC3CCN(CC(=O)N4CCC(c5ccc(NC6CCC(=O)NC6=O)cc5C(F)(F)F)CC4)CC3)c(OC(F)(F)F)c2)c2ccncc2c1=O ; COc1cc(-c2cn(C)c(=O)c3cnccc23)cc(F)c1CN1CCC(CCN2CCC(c3ccc(NC4CCC(=O)NC4=O)cc3C(F)(F)F)CC2)CC1 ; COc1cc(-c2cn(C)c(=O)c3cnccc23)cc(Cl)c1CN1CCC(CCN2CCC(c3ccc(NC4CCC(=O)NC4=O)cc3C(F)(F)F)CC2)CC1]

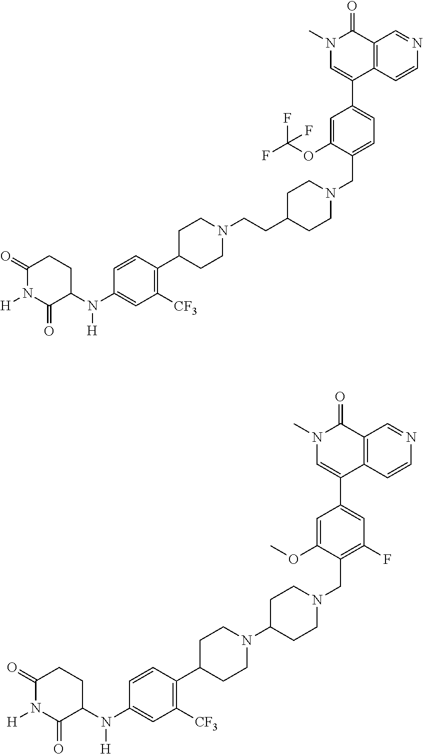 [SMILES: Cn1cc(-c2ccc(CN3CCC(CCN4CCC(c5ccc(NC6CCC(=O)NC6=O)cc5C(F)(F)F)CC4)CC3)c(OC(F)(F)F)c2)c2ccncc2c1=O ; COc1cc(-c2cn(C)c(=O)c3cnccc23)cc(F)c1CN1CCC(N2CCC(c3ccc(NC4CCC(=O)NC4=O)cc3C(F)(F)F)CC2)CC1]

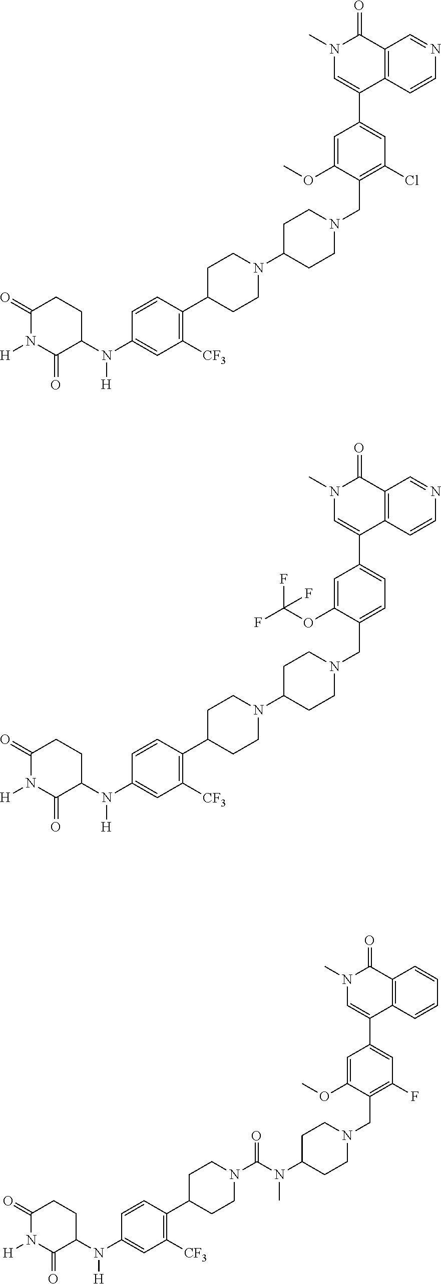 [SMILES: COc1cc(-c2cn(C)c(=O)c3cnccc23)cc(Cl)c1CN1CCC(N2CCC(c3ccc(NC4CCC(=O)NC4=O)cc3C(F)(F)F)CC2)CC1 ; Cn1cc(-c2ccc(CN3CCC(N4CCC(c5ccc(NC6CCC(=O)NC6=O)cc5C(F)(F)F)CC4)CC3)c(OC(F)(F)F)c2)c2ccncc2c1=O ; COc1cc(-c2cn(C)c(=O)c3ccccc23)cc(F)c1CN1CCC(N(C)C(=O)N2CCC(c3ccc(NC4CCC(=O)NC4=O)cc3C(F)(F)F)CC2)CC1]

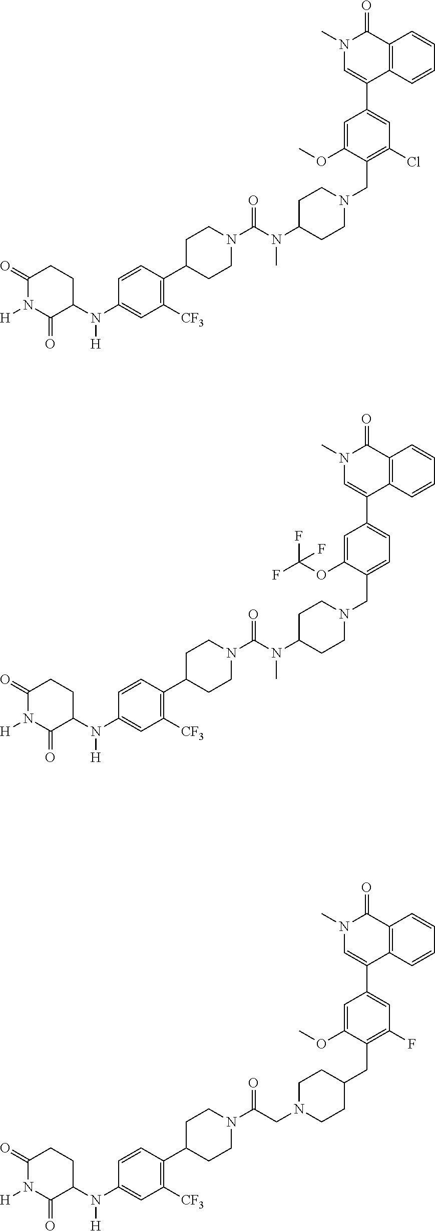 [SMILES: COc1cc(-c2cn(C)c(=O)c3ccccc23)cc(Cl)c1CN1CCC(N(C)C(=O)N2CCC(c3ccc(NC4CCC(=O)NC4=O)cc3C(F)(F)F)CC2)CC1 ; CN(C(=O)N1CCC(c2ccc(NC3CCC(=O)NC3=O)cc2C(F)(F)F)CC1)C1CCN(Cc2ccc(-c3cn(C)c(=O)c4ccccc34)cc2OC(F)(F)F)CC1 ; COc1cc(-c2cn(C)c(=O)c3ccccc23)cc(F)c1CC1CCN(CC(=O)N2CCC(c3ccc(NC4CCC(=O)NC4=O)cc3C(F)(F)F)CC2)CC1]

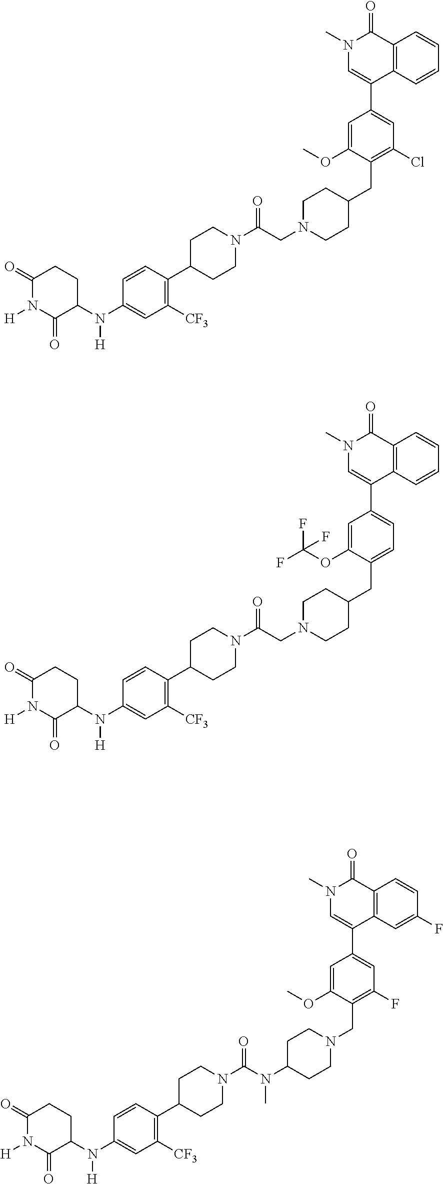 [SMILES: COc1cc(-c2cn(C)c(=O)c3ccccc23)cc(Cl)c1CC1CCN(CC(=O)N2CCC(c3ccc(NC4CCC(=O)NC4=O)cc3C(F)(F)F)CC2)CC1 ; Cn1cc(-c2ccc(CC3CCN(CC(=O)N4CCC(c5ccc(NC6CCC(=O)NC6=O)cc5C(F)(F)F)CC4)CC3)c(OC(F)(F)F)c2)c2ccccc2c1=O ; COc1cc(-c2cn(C)c(=O)c3ccc(F)cc23)cc(F)c1CN1CCC(N(C)C(=O)N2CCC(c3ccc(NC4CCC(=O)NC4=O)cc3C(F)(F)F)CC2)CC1]

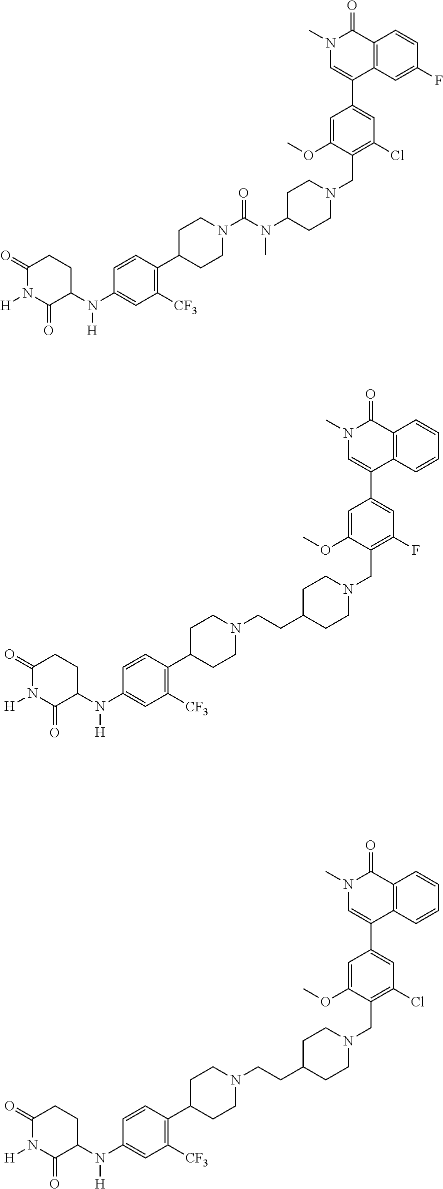 [SMILES: COc1cc(-c2cn(C)c(=O)c3ccc(F)cc23)cc(Cl)c1CN1CCC(N(C)C(=O)N2CCC(c3ccc(NC4CCC(=O)NC4=O)cc3C(F)(F)F)CC2)CC1 ; COc1cc(-c2cn(C)c(=O)c3ccccc23)cc(F)c1CN1CCC(CCN2CCC(c3ccc(NC4CCC(=O)NC4=O)cc3C(F)(F)F)CC2)CC1 ; COc1cc(-c2cn(C)c(=O)c3ccccc23)cc(Cl)c1CN1CCC(CCN2CCC(c3ccc(NC4CCC(=O)NC4=O)cc3C(F)(F)F)CC2)CC1]

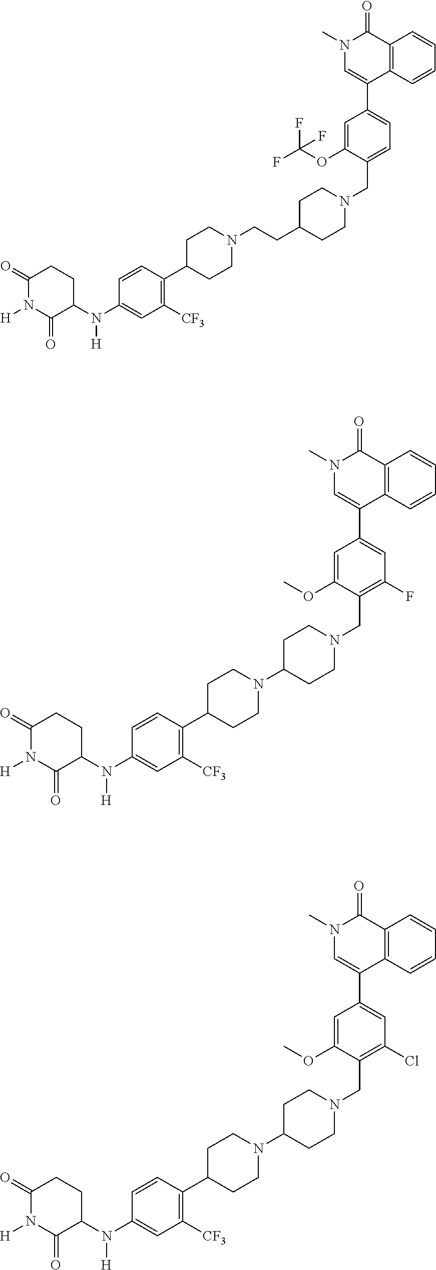 [SMILES: Cn1cc(-c2ccc(CN3CCC(CCN4CCC(c5ccc(NC6CCC(=O)NC6=O)cc5C(F)(F)F)CC4)CC3)c(OC(F)(F)F)c2)c2ccccc2c1=O ; COc1cc(-c2cn(C)c(=O)c3ccccc23)cc(F)c1CN1CCC(N2CCC(c3ccc(NC4CCC(=O)NC4=O)cc3C(F)(F)F)CC2)CC1 ; COc1cc(-c2cn(C)c(=O)c3ccccc23)cc(Cl)c1CN1CCC(N2CCC(c3ccc(NC4CCC(=O)NC4=O)cc3C(F)(F)F)CC2)CC1]

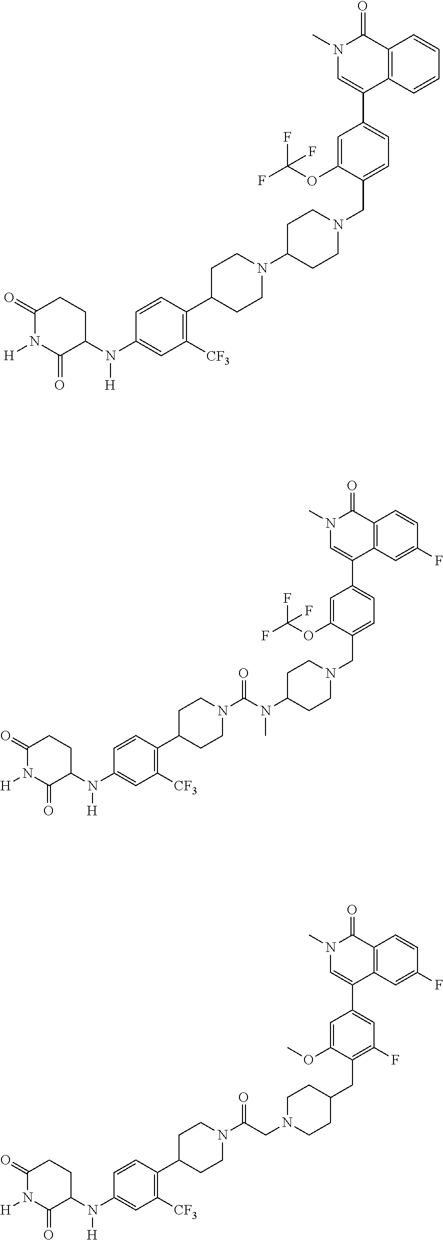 [SMILES: Cn1cc(-c2ccc(CN3CCC(N4CCC(c5ccc(NC6CCC(=O)NC6=O)cc5C(F)(F)F)CC4)CC3)c(OC(F)(F)F)c2)c2ccccc2c1=O ; CN(C(=O)N1CCC(c2ccc(NC3CCC(=O)NC3=O)cc2C(F)(F)F)CC1)C1CCN(Cc2ccc(-c3cn(C)c(=O)c4ccc(F)cc34)cc2OC(F)(F)F)CC1 ; COc1cc(-c2cn(C)c(=O)c3ccc(F)cc23)cc(F)c1CC1CCN(CC(=O)N2CCC(c3ccc(NC4CCC(=O)NC4=O)cc3C(F)(F)F)CC2)CC1]

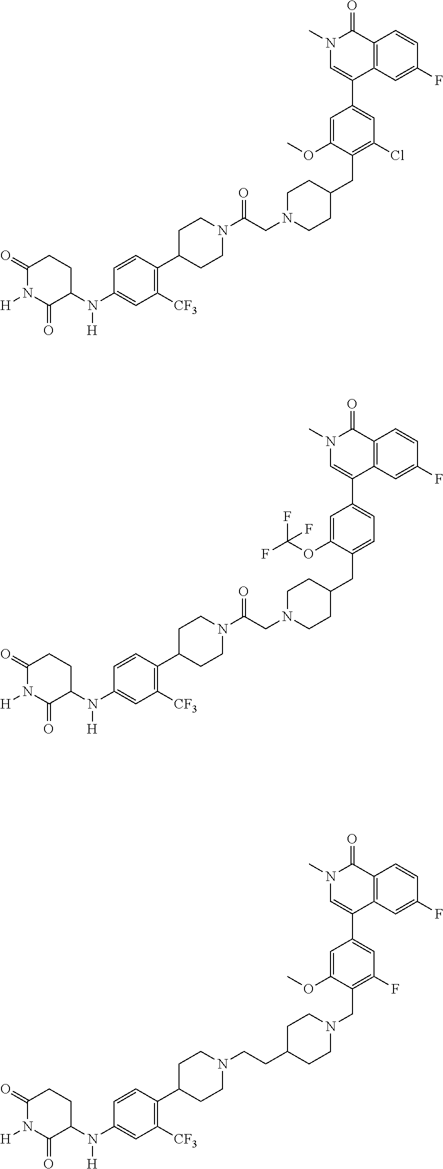 [SMILES: COc1cc(-c2cn(C)c(=O)c3ccc(F)cc23)cc(Cl)c1CC1CCN(CC(=O)N2CCC(c3ccc(NC4CCC(=O)NC4=O)cc3C(F)(F)F)CC2)CC1 ; Cn1cc(-c2ccc(CC3CCN(CC(=O)N4CCC(c5ccc(NC6CCC(=O)NC6=O)cc5C(F)(F)F)CC4)CC3)c(OC(F)(F)F)c2)c2cc(F)ccc2c1=O ; COc1cc(-c2cn(C)c(=O)c3ccc(F)cc23)cc(F)c1CN1CCC(CCN2CCC(c3ccc(NC4CCC(=O)NC4=O)cc3C(F)(F)F)CC2)CC1]

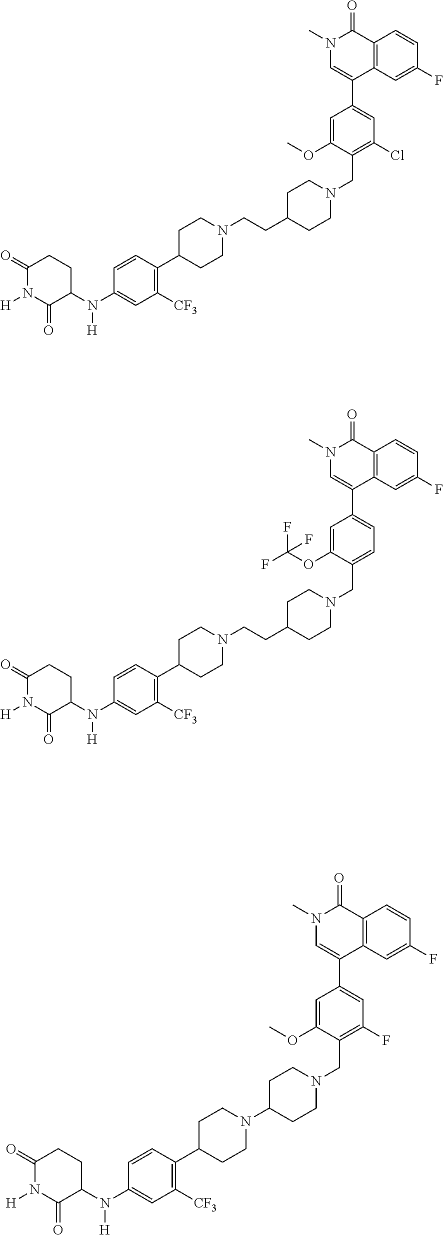 [SMILES: COc1cc(-c2cn(C)c(=O)c3ccc(F)cc23)cc(Cl)c1CN1CCC(CCN2CCC(c3ccc(NC4CCC(=O)NC4=O)cc3C(F)(F)F)CC2)CC1 ; Cn1cc(-c2ccc(CN3CCC(CCN4CCC(c5ccc(NC6CCC(=O)NC6=O)cc5C(F)(F)F)CC4)CC3)c(OC(F)(F)F)c2)c2cc(F)ccc2c1=O ; COc1cc(-c2cn(C)c(=O)c3ccc(F)cc23)cc(F)c1CN1CCC(N2CCC(c3ccc(NC4CCC(=O)NC4=O)cc3C(F)(F)F)CC2)CC1]

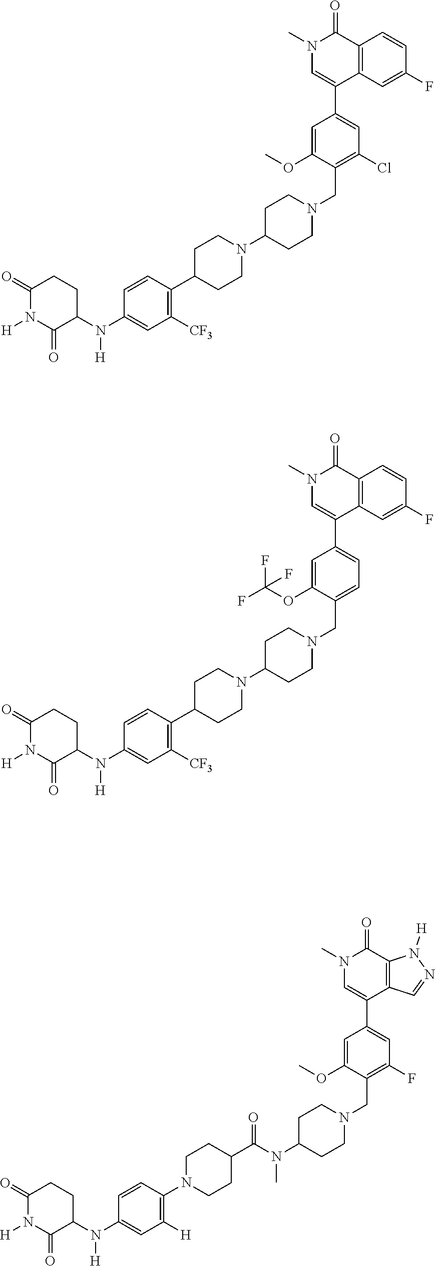 [SMILES: COc1cc(-c2cn(C)c(=O)c3ccc(F)cc23)cc(Cl)c1CN1CCC(N2CCC(c3ccc(NC4CCC(=O)NC4=O)cc3C(F)(F)F)CC2)CC1 ; Cn1cc(-c2ccc(CN3CCC(N4CCC(c5ccc(NC6CCC(=O)NC6=O)cc5C(F)(F)F)CC4)CC3)c(OC(F)(F)F)c2)c2cc(F)ccc2c1=O ; COc1cc(-c2cn(C)c(=O)c3[nH]ncc23)cc(F)c1CN1CCC(N(C)C(=O)C2CCN(c3ccc(NC4CCC(=O)NC4=O)cc3)CC2)CC1]

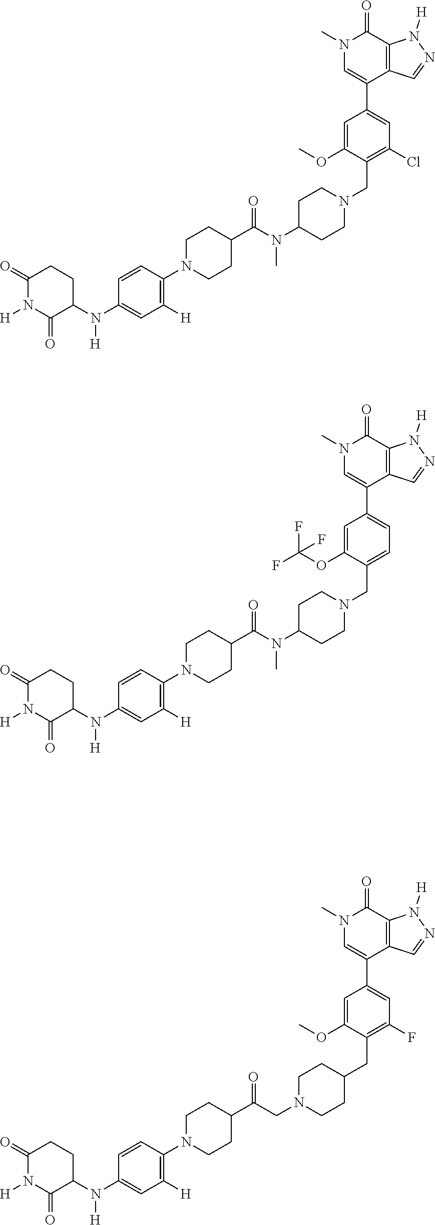 [SMILES: COc1cc(-c2cn(C)c(=O)c3[nH]ncc23)cc(Cl)c1CN1CCC(N(C)C(=O)C2CCN(c3ccc(NC4CCC(=O)NC4=O)cc3)CC2)CC1 ; CN(C(=O)C1CCN(c2ccc(NC3CCC(=O)NC3=O)cc2)CC1)C1CCN(Cc2ccc(-c3cn(C)c(=O)c4[nH]ncc34)cc2OC(F)(F)F)CC1 ; COc1cc(-c2cn(C)c(=O)c3[nH]ncc23)cc(F)c1CC1CCN(CC(=O)C2CCN(c3ccc(NC4CCC(=O)NC4=O)cc3)CC2)CC1]

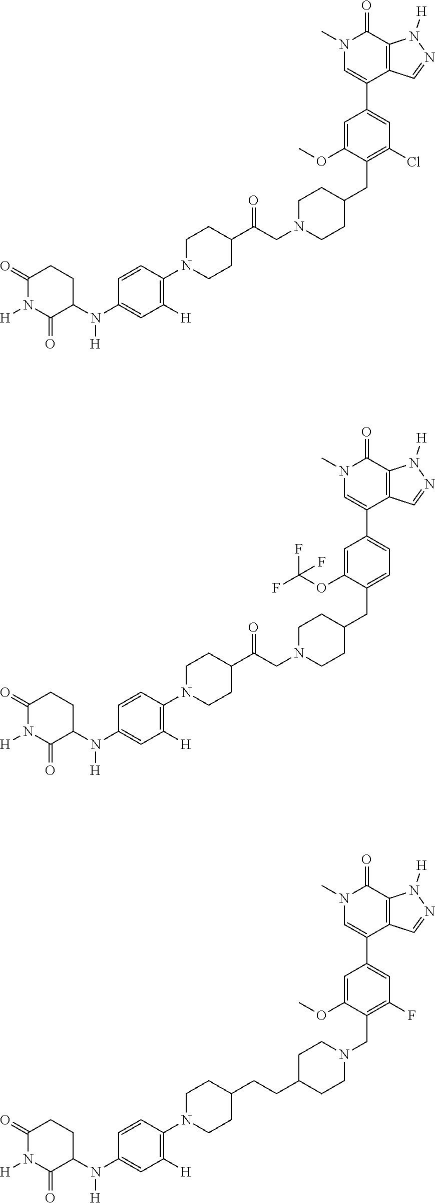 [SMILES: COc1cc(-c2cn(C)c(=O)c3[nH]ncc23)cc(Cl)c1CC1CCN(CC(=O)C2CCN(c3ccc(NC4CCC(=O)NC4=O)cc3)CC2)CC1 ; Cn1cc(-c2ccc(CC3CCN(CC(=O)C4CCN(c5ccc(NC6CCC(=O)NC6=O)cc5)CC4)CC3)c(OC(F)(F)F)c2)c2cn[nH]c2c1=O ; COc1cc(-c2cn(C)c(=O)c3[nH]ncc23)cc(F)c1CN1CCC(CCC2CCN(c3ccc(NC4CCC(=O)NC4=O)cc3)CC2)CC1]

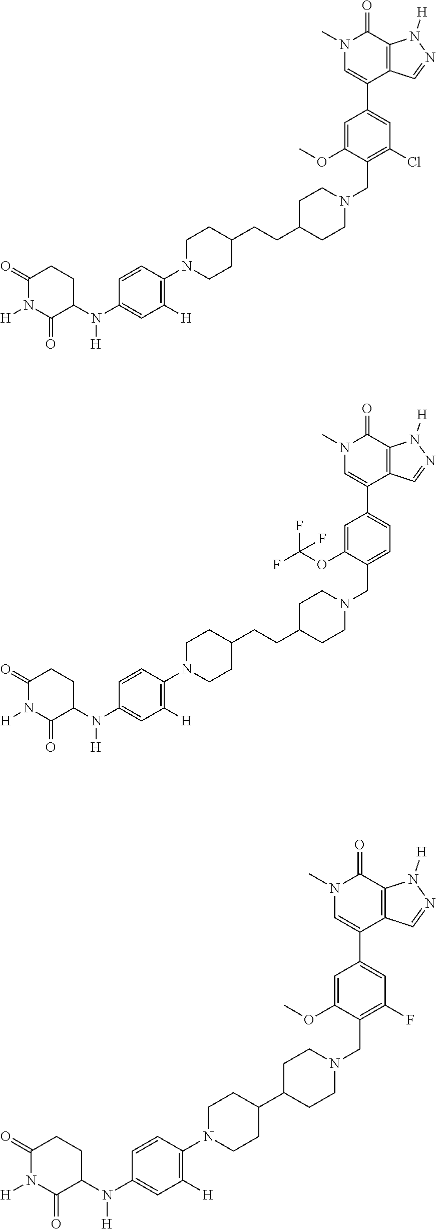 [SMILES: COc1cc(-c2cn(C)c(=O)c3[nH]ncc23)cc(Cl)c1CN1CCC(CCC2CCN(c3ccc(NC4CCC(=O)NC4=O)cc3)CC2)CC1 ; Cn1cc(-c2ccc(CN3CCC(CCC4CCN(c5ccc(NC6CCC(=O)NC6=O)cc5)CC4)CC3)c(OC(F)(F)F)c2)c2cn[nH]c2c1=O ; COc1cc(-c2cn(C)c(=O)c3[nH]ncc23)cc(F)c1CN1CCC(C2CCN(c3ccc(NC4CCC(=O)NC4=O)cc3)CC2)CC1]

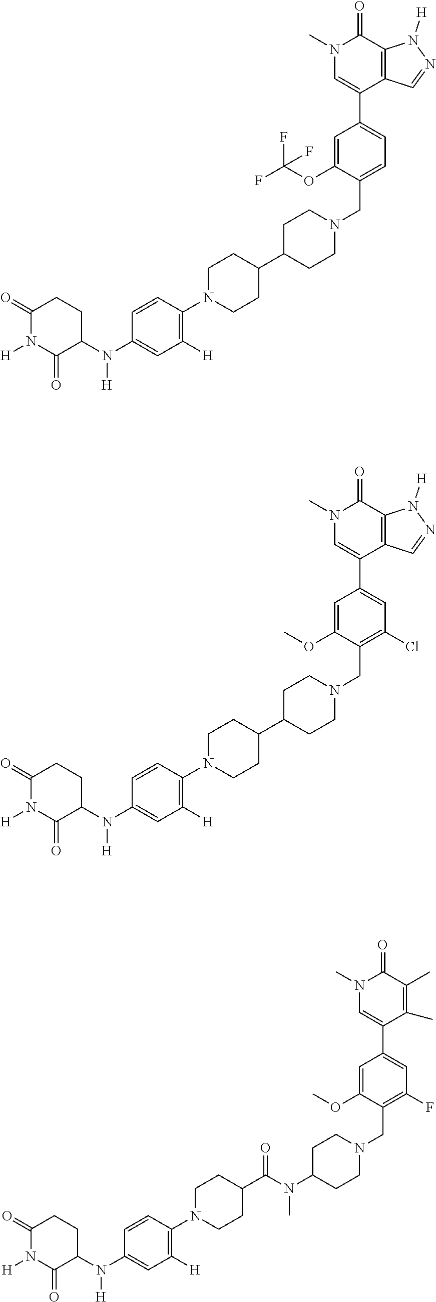 [SMILES: Cn1cc(-c2ccc(CN3CCC(C4CCN(c5ccc(NC6CCC(=O)NC6=O)cc5)CC4)CC3)c(OC(F)(F)F)c2)c2cn[nH]c2c1=O ; COc1cc(-c2cn(C)c(=O)c3[nH]ncc23)cc(Cl)c1CN1CCC(C2CCN(c3ccc(NC4CCC(=O)NC4=O)cc3)CC2)CC1 ; COc1cc(-c2cn(C)c(=O)c(C)c2C)cc(F)c1CN1CCC(N(C)C(=O)C2CCN(c3ccc(NC4CCC(=O)NC4=O)cc3)CC2)CC1]

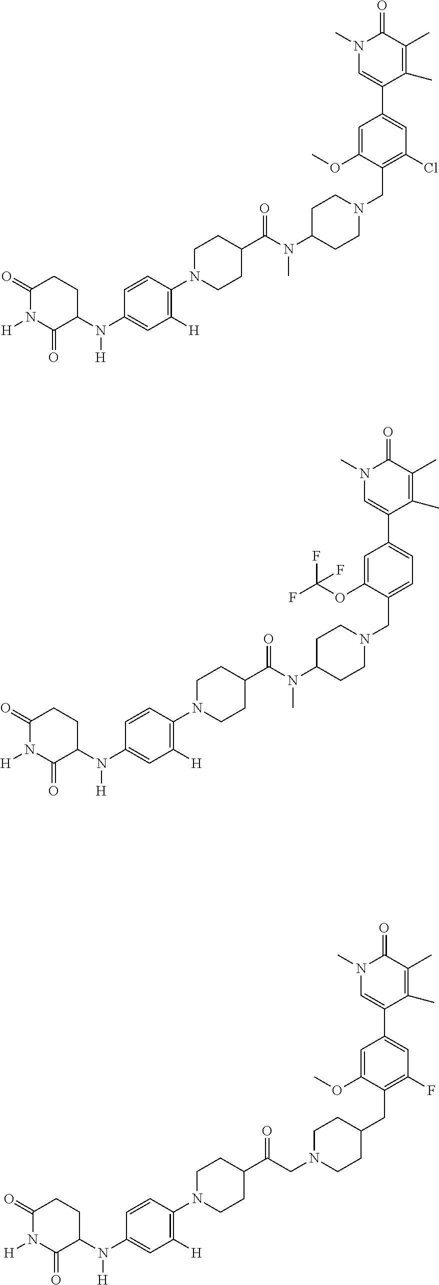 [SMILES: COc1cc(-c2cn(C)c(=O)c(C)c2C)cc(Cl)c1CN1CCC(N(C)C(=O)C2CCN(c3ccc(NC4CCC(=O)NC4=O)cc3)CC2)CC1 ; Cc1c(-c2ccc(CN3CCC(N(C)C(=O)C4CCN(c5ccc(NC6CCC(=O)NC6=O)cc5)CC4)CC3)c(OC(F)(F)F)c2)cn(C)c(=O)c1C ; COc1cc(-c2cn(C)c(=O)c(C)c2C)cc(F)c1CC1CCN(CC(=O)C2CCN(c3ccc(NC4CCC(=O)NC4=O)cc3)CC2)CC1]

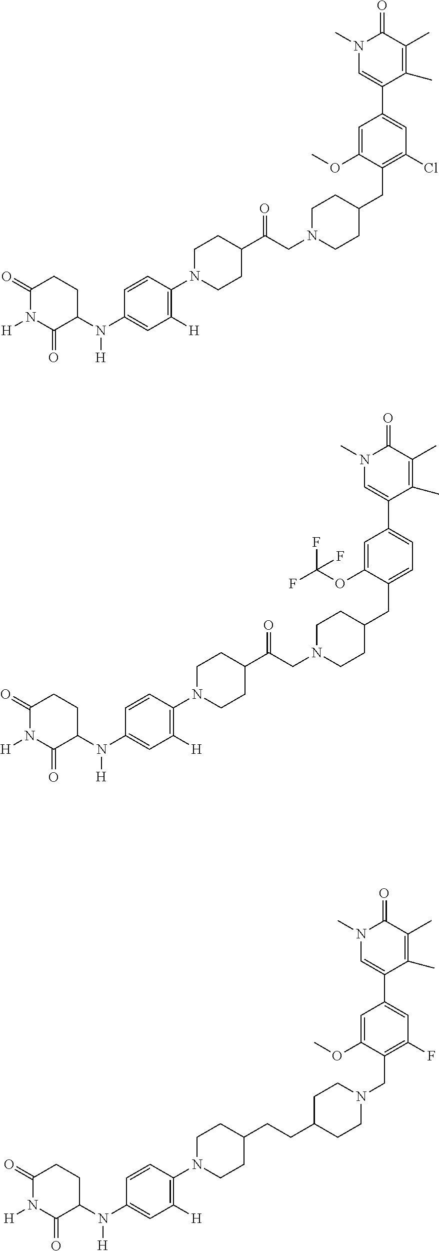 [SMILES: COc1cc(-c2cn(C)c(=O)c(C)c2C)cc(Cl)c1CC1CCN(CC(=O)C2CCN(c3ccc(NC4CCC(=O)NC4=O)cc3)CC2)CC1 ; Cc1c(-c2ccc(CC3CCN(CC(=O)C4CCN(c5ccc(NC6CCC(=O)NC6=O)cc5)CC4)CC3)c(OC(F)(F)F)c2)cn(C)c(=O)c1C ; COc1cc(-c2cn(C)c(=O)c(C)c2C)cc(F)c1CN1CCC(CCC2CCN(c3ccc(NC4CCC(=O)NC4=O)cc3)CC2)CC1]

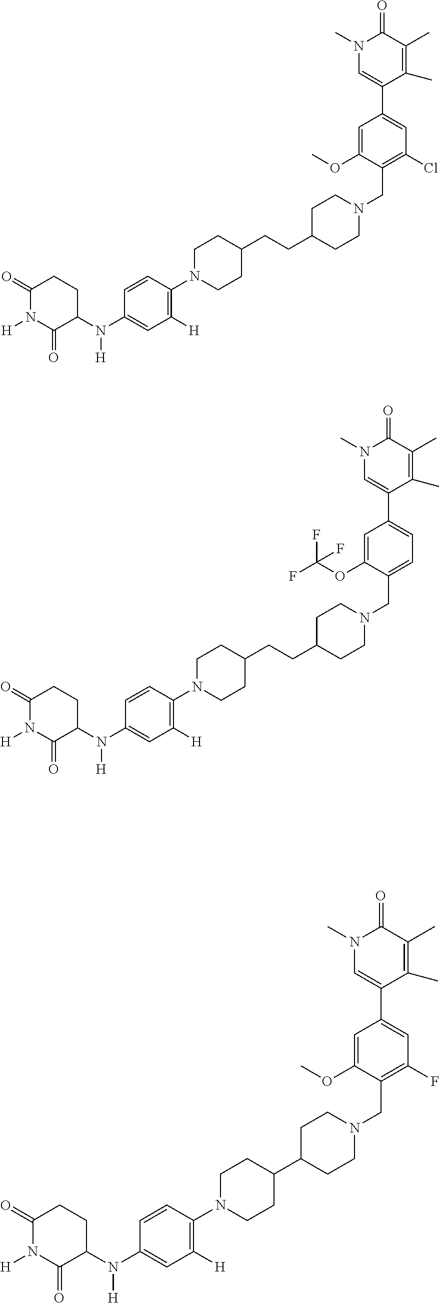 [SMILES: COc1cc(-c2cn(C)c(=O)c(C)c2C)cc(Cl)c1CN1CCC(CCC2CCN(c3ccc(NC4CCC(=O)NC4=O)cc3)CC2)CC1 ; Cc1c(-c2ccc(CN3CCC(CCC4CCN(c5ccc(NC6CCC(=O)NC6=O)cc5)CC4)CC3)c(OC(F)(F)F)c2)cn(C)c(=O)c1C ; COc1cc(-c2cn(C)c(=O)c(C)c2C)cc(F)c1CN1CCC(C2CCN(c3ccc(NC4CCC(=O)NC4=O)cc3)CC2)CC1]

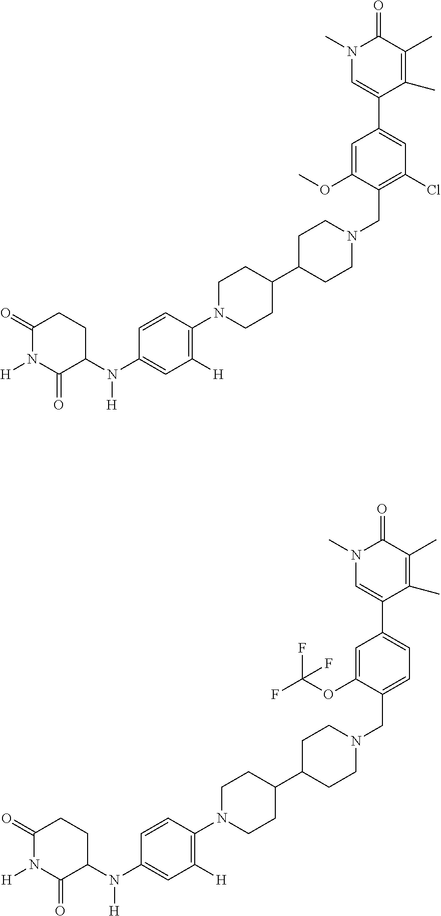 [SMILES: COc1cc(-c2cn(C)c(=O)c(C)c2C)cc(Cl)c1CN1CCC(C2CCN(c3ccc(NC4CCC(=O)NC4=O)cc3)CC2)CC1 ; Cc1c(-c2ccc(CN3CCC(C4CCN(c5ccc(NC6CCC(=O)NC6=O)cc5)CC4)CC3)c(OC(F)(F)F)c2)cn(C)c(=O)c1C]

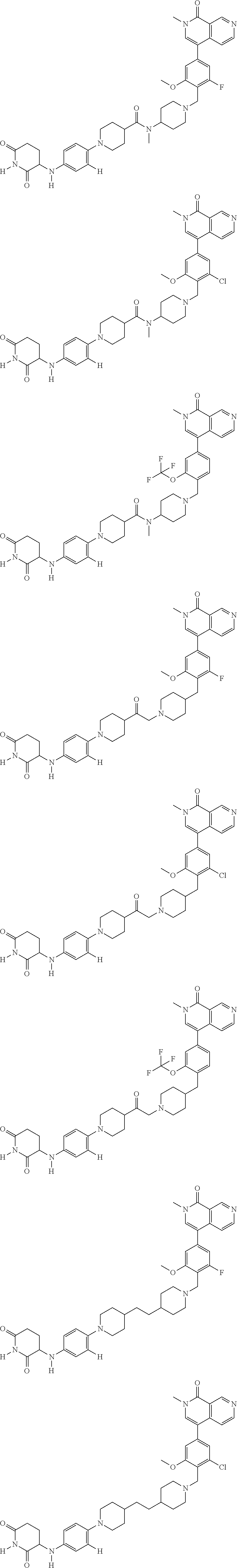

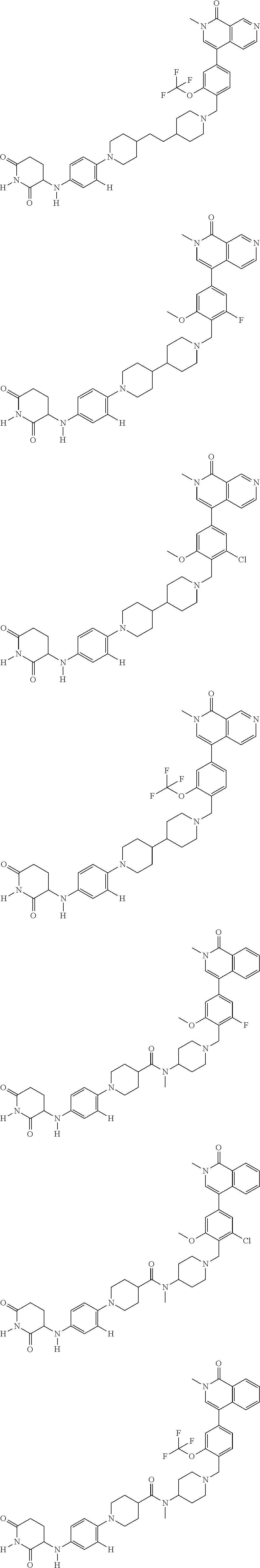

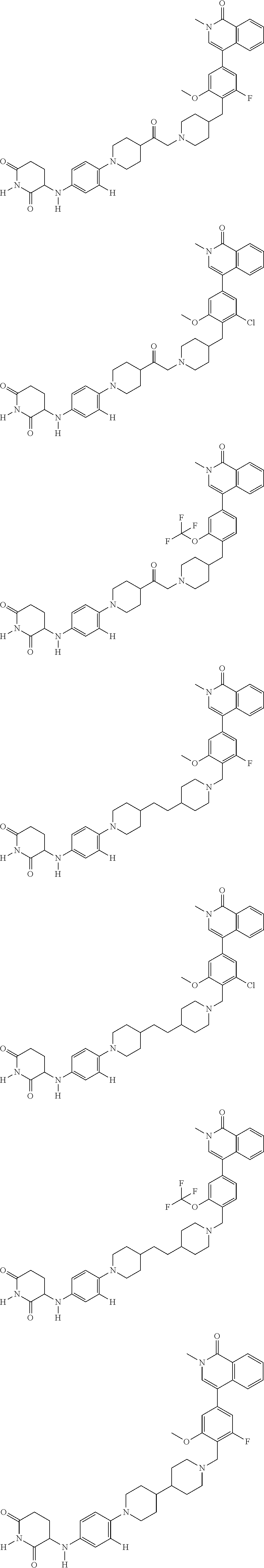

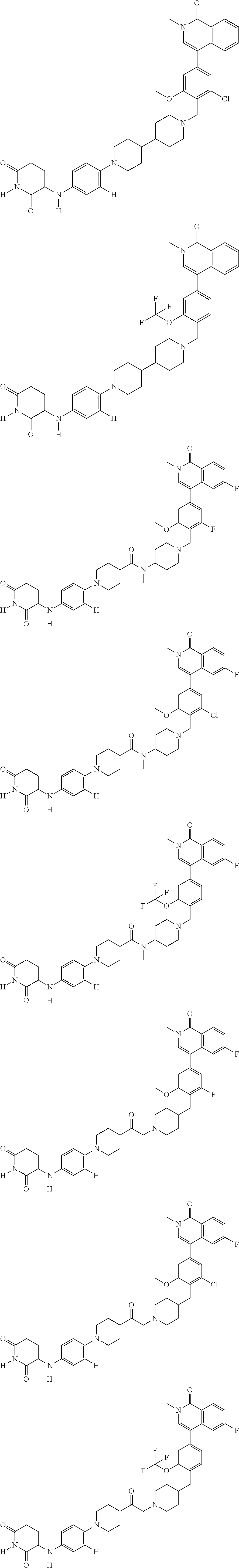

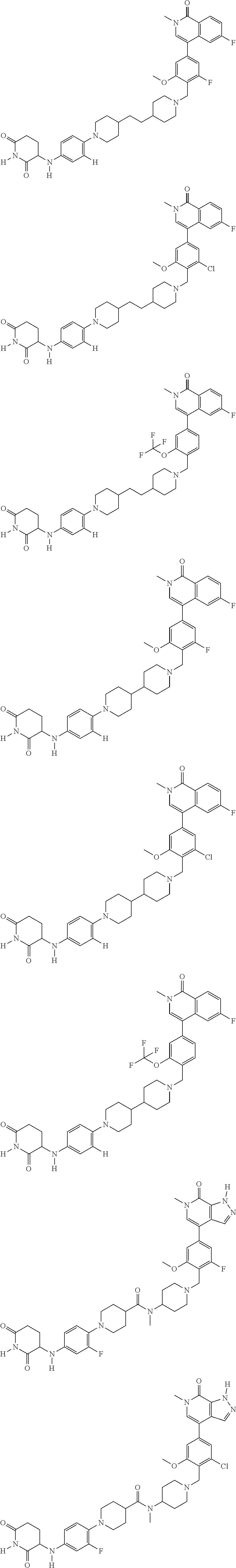

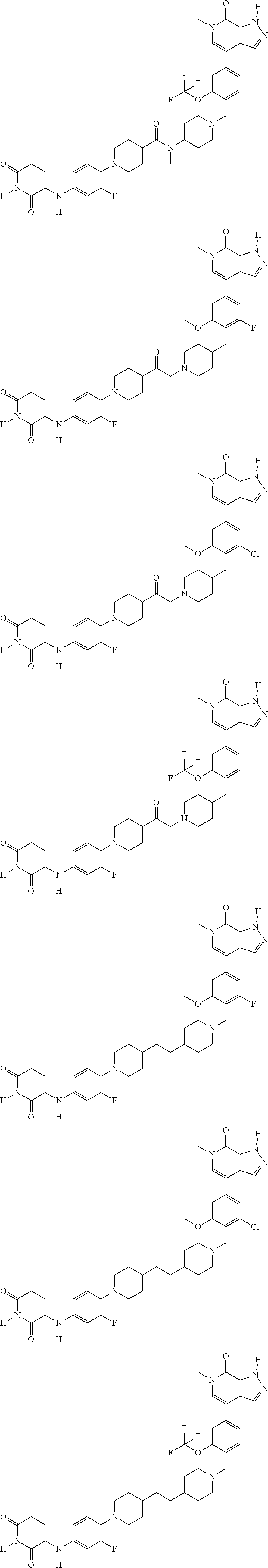

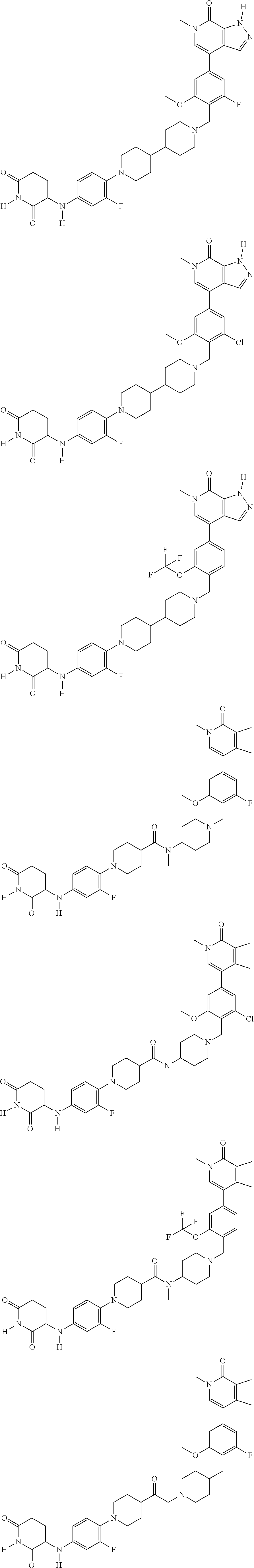

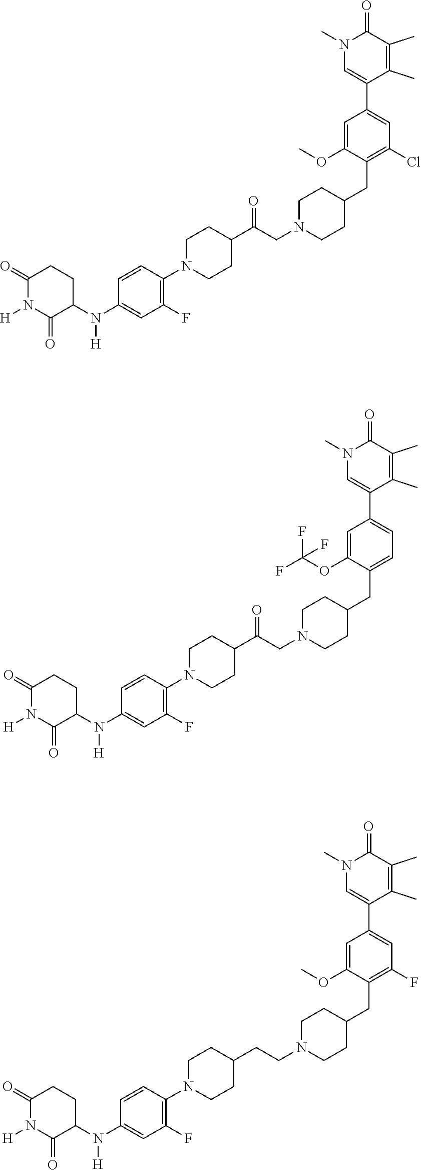 [SMILES: COc1cc(-c2cn(C)c(=O)c(C)c2C)cc(Cl)c1CC1CCN(CC(=O)C2CCN(c3ccc(NC4CCC(=O)NC4=O)cc3F)CC2)CC1 ; Cc1c(-c2ccc(CC3CCN(CC(=O)C4CCN(c5ccc(NC6CCC(=O)NC6=O)cc5F)CC4)CC3)c(OC(F)(F)F)c2)cn(C)c(=O)c1C ; COc1cc(-c2cn(C)c(=O)c(C)c2C)cc(F)c1CC1CCN(CCC2CCN(c3ccc(NC4CCC(=O)NC4=O)cc3F)CC2)CC1]

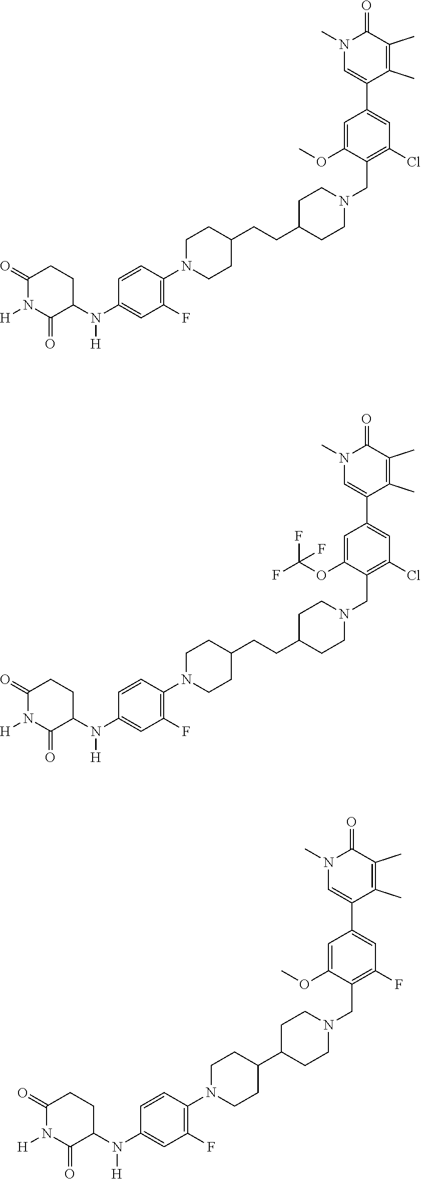 [SMILES: COc1cc(-c2cn(C)c(=O)c(C)c2C)cc(Cl)c1CN1CCC(CCC2CCN(c3ccc(NC4CCC(=O)NC4=O)cc3F)CC2)CC1 ; Cc1c(-c2cc(Cl)c(CN3CCC(CCC4CCN(c5ccc(NC6CCC(=O)NC6=O)cc5F)CC4)CC3)c(OC(F)(F)F)c2)cn(C)c(=O)c1C ; COc1cc(-c2cn(C)c(=O)c(C)c2C)cc(F)c1CN1CCC(C2CCN(c3ccc(NC4CCC(=O)NC4=O)cc3F)CC2)CC1]

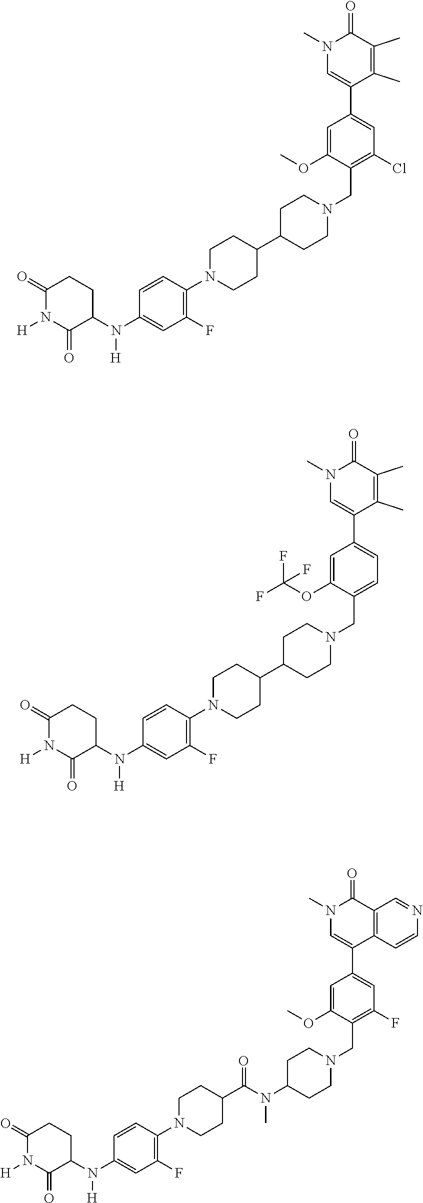 [SMILES: COc1cc(-c2cn(C)c(=O)c(C)c2C)cc(Cl)c1CN1CCC(C2CCN(c3ccc(NC4CCC(=O)NC4=O)cc3F)CC2)CC1 ; Cc1c(-c2ccc(CN3CCC(C4CCN(c5ccc(NC6CCC(=O)NC6=O)cc5F)CC4)CC3)c(OC(F)(F)F)c2)cn(C)c(=O)c1C ; COc1cc(-c2cn(C)c(=O)c3cnccc23)cc(F)c1CN1CCC(N(C)C(=O)C2CCN(c3ccc(NC4CCC(=O)NC4=O)cc3F)CC2)CC1]

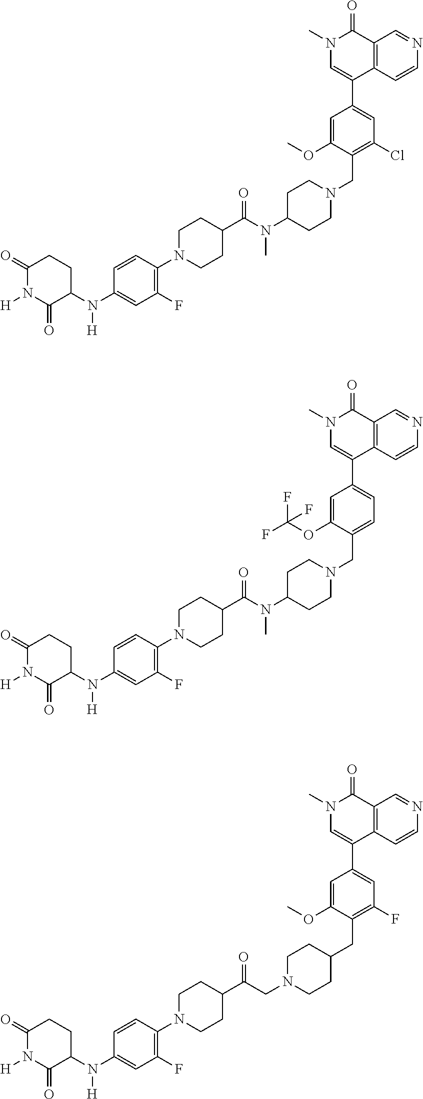 [SMILES: COc1cc(-c2cn(C)c(=O)c3cnccc23)cc(Cl)c1CN1CCC(N(C)C(=O)C2CCN(c3ccc(NC4CCC(=O)NC4=O)cc3F)CC2)CC1 ; CN(C(=O)C1CCN(c2ccc(NC3CCC(=O)NC3=O)cc2F)CC1)C1CCN(Cc2ccc(-c3cn(C)c(=O)c4cnccc34)cc2OC(F)(F)F)CC1 ; COc1cc(-c2cn(C)c(=O)c3cnccc23)cc(F)c1CC1CCN(CC(=O)C2CCN(c3ccc(NC4CCC(=O)NC4=O)cc3F)CC2)CC1]

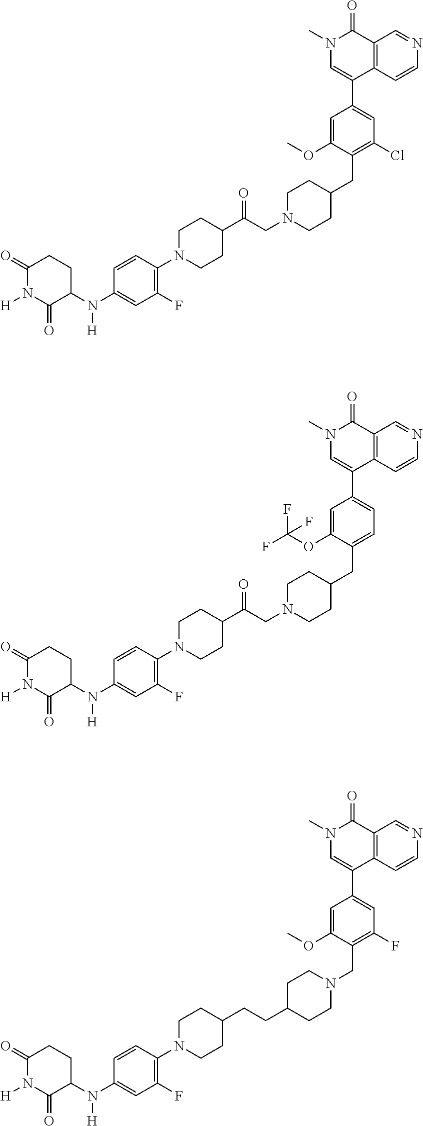 [SMILES: COc1cc(-c2cn(C)c(=O)c3cnccc23)cc(Cl)c1CC1CCN(CC(=O)C2CCN(c3ccc(NC4CCC(=O)NC4=O)cc3F)CC2)CC1 ; Cn1cc(-c2ccc(CC3CCN(CC(=O)C4CCN(c5ccc(NC6CCC(=O)NC6=O)cc5F)CC4)CC3)c(OC(F)(F)F)c2)c2ccncc2c1=O ; COc1cc(-c2cn(C)c(=O)c3cnccc23)cc(F)c1CN1CCC(CCC2CCN(c3ccc(NC4CCC(=O)NC4=O)cc3F)CC2)CC1]

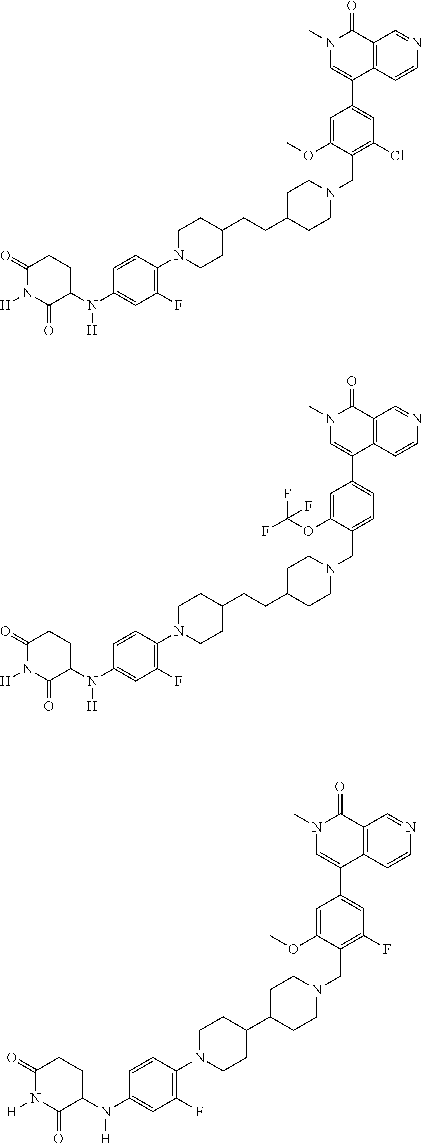 [SMILES: COc1cc(-c2cn(C)c(=O)c3cnccc23)cc(Cl)c1CN1CCC(CCC2CCN(c3ccc(NC4CCC(=O)NC4=O)cc3F)CC2)CC1 ; Cn1cc(-c2ccc(CN3CCC(CCC4CCN(c5ccc(NC6CCC(=O)NC6=O)cc5F)CC4)CC3)c(OC(F)(F)F)c2)c2ccncc2c1=O ; COc1cc(-c2cn(C)c(=O)c3cnccc23)cc(F)c1CN1CCC(C2CCN(c3ccc(NC4CCC(=O)NC4=O)cc3F)CC2)CC1]

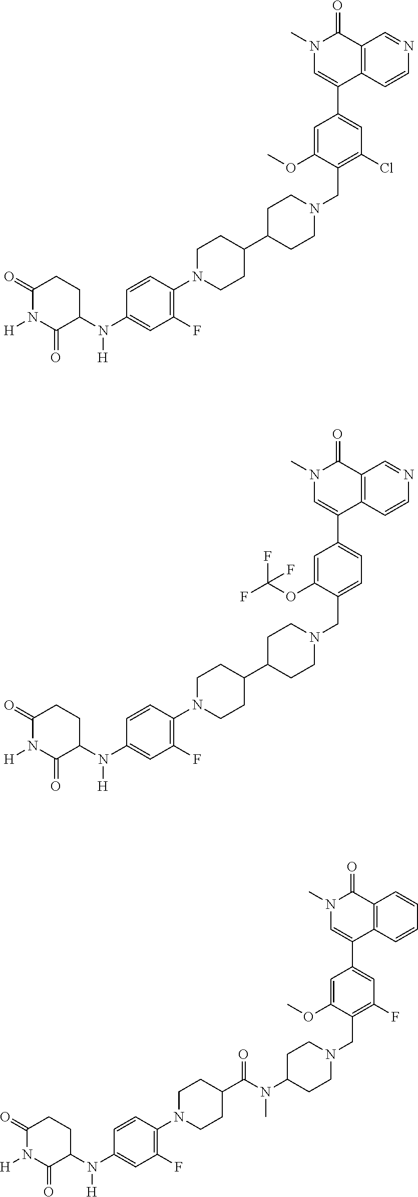 [SMILES: COc1cc(-c2cn(C)c(=O)c3cnccc23)cc(Cl)c1CN1CCC(C2CCN(c3ccc(NC4CCC(=O)NC4=O)cc3F)CC2)CC1 ; Cn1cc(-c2ccc(CN3CCC(C4CCN(c5ccc(NC6CCC(=O)NC6=O)cc5F)CC4)CC3)c(OC(F)(F)F)c2)c2ccncc2c1=O ; COc1cc(-c2cn(C)c(=O)c3ccccc23)cc(F)c1CN1CCC(N(C)C(=O)C2CCN(c3ccc(NC4CCC(=O)NC4=O)cc3F)CC2)CC1]

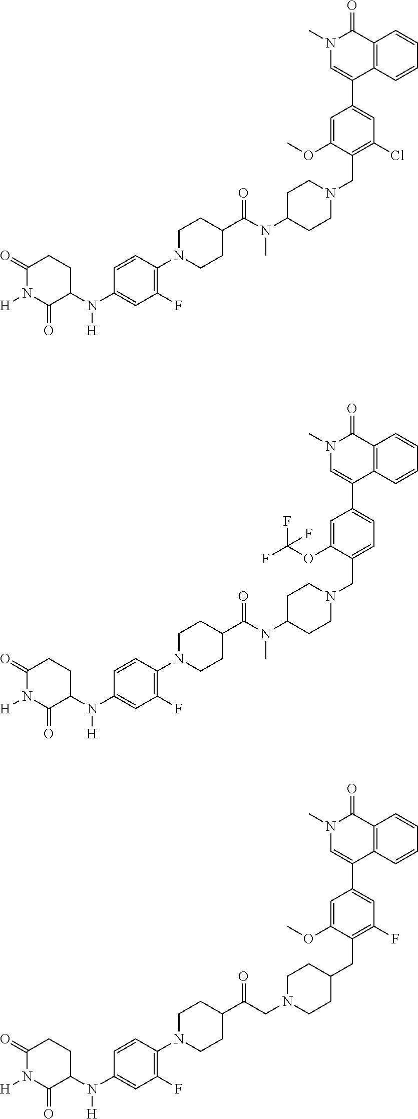 [SMILES: COc1cc(-c2cn(C)c(=O)c3ccccc23)cc(Cl)c1CN1CCC(N(C)C(=O)C2CCN(c3ccc(NC4CCC(=O)NC4=O)cc3F)CC2)CC1 ; CN(C(=O)C1CCN(c2ccc(NC3CCC(=O)NC3=O)cc2F)CC1)C1CCN(Cc2ccc(-c3cn(C)c(=O)c4ccccc34)cc2OC(F)(F)F)CC1 ; COc1cc(-c2cn(C)c(=O)c3ccccc23)cc(F)c1CC1CCN(CC(=O)C2CCN(c3ccc(NC4CCC(=O)NC4=O)cc3F)CC2)CC1]

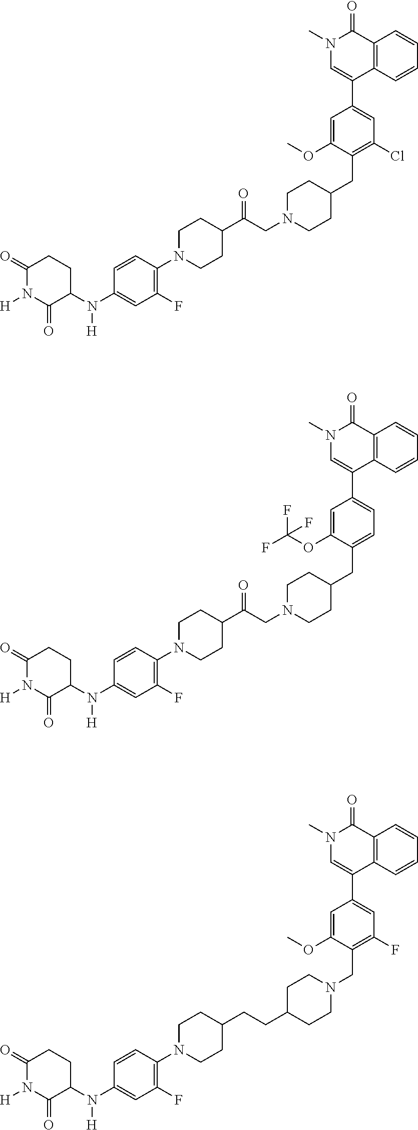 [SMILES: COc1cc(-c2cn(C)c(=O)c3ccccc23)cc(Cl)c1CC1CCN(CC(=O)C2CCN(c3ccc(NC4CCC(=O)NC4=O)cc3F)CC2)CC1 ; Cn1cc(-c2ccc(CC3CCN(CC(=O)C4CCN(c5ccc(NC6CCC(=O)NC6=O)cc5F)CC4)CC3)c(OC(F)(F)F)c2)c2ccccc2c1=O ; COc1cc(-c2cn(C)c(=O)c3ccccc23)cc(F)c1CN1CCC(CCC2CCN(c3ccc(NC4CCC(=O)NC4=O)cc3F)CC2)CC1]

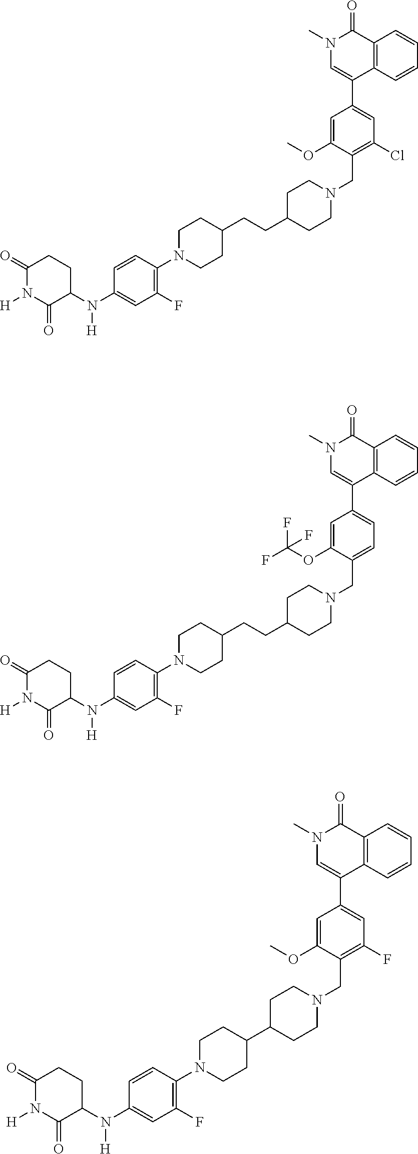 [SMILES: COc1cc(-c2cn(C)c(=O)c3ccccc23)cc(Cl)c1CN1CCC(CCC2CCN(c3ccc(NC4CCC(=O)NC4=O)cc3F)CC2)CC1 ; Cn1cc(-c2ccc(CN3CCC(CCC4CCN(c5ccc(NC6CCC(=O)NC6=O)cc5F)CC4)CC3)c(OC(F)(F)F)c2)c2ccccc2c1=O ; COc1cc(-c2cn(C)c(=O)c3ccccc23)cc(F)c1CN1CCC(C2CCN(c3ccc(NC4CCC(=O)NC4=O)cc3F)CC2)CC1]

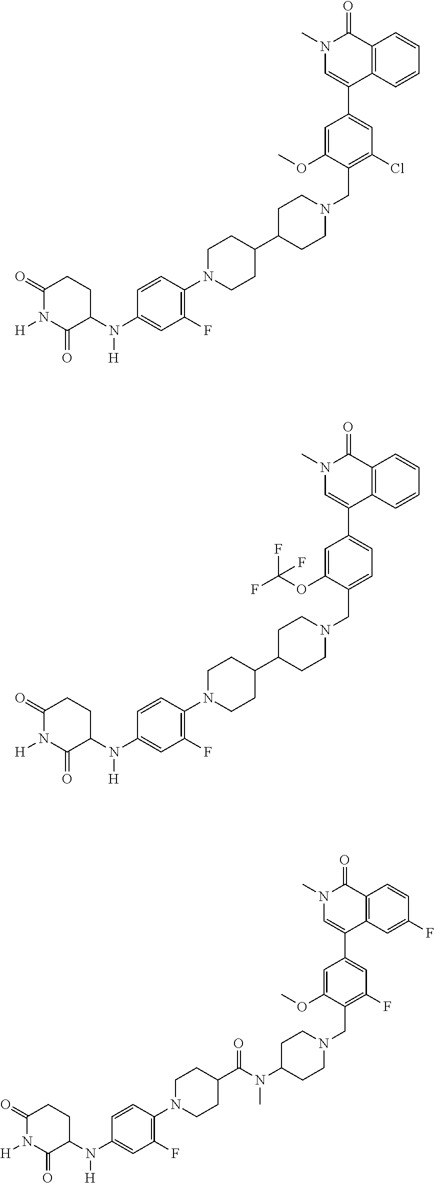 [SMILES: COc1cc(-c2cn(C)c(=O)c3ccccc23)cc(Cl)c1CN1CCC(C2CCN(c3ccc(NC4CCC(=O)NC4=O)cc3F)CC2)CC1 ; Cn1cc(-c2ccc(CN3CCC(C4CCN(c5ccc(NC6CCC(=O)NC6=O)cc5F)CC4)CC3)c(OC(F)(F)F)c2)c2ccccc2c1=O ; COc1cc(-c2cn(C)c(=O)c3ccc(F)cc23)cc(F)c1CN1CCC(N(C)C(=O)C2CCN(c3ccc(NC4CCC(=O)NC4=O)cc3F)CC2)CC1]

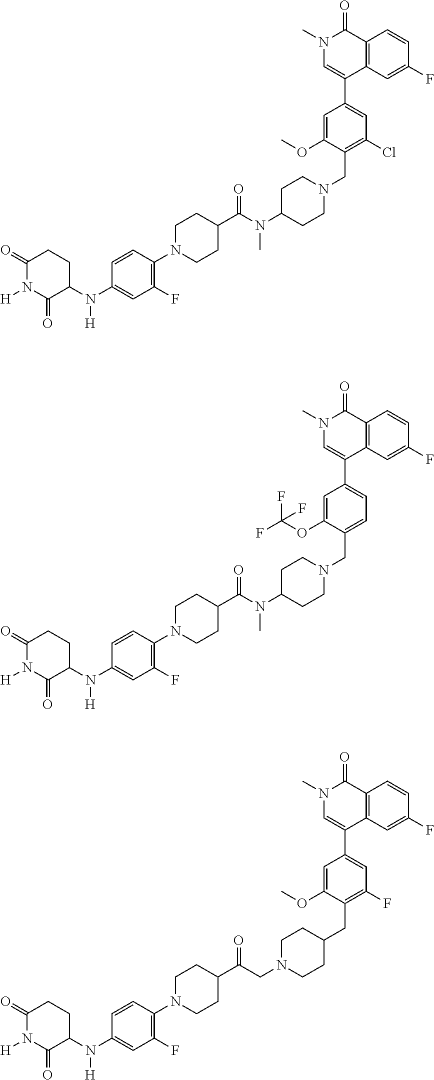 [SMILES: COc1cc(-c2cn(C)c(=O)c3ccc(F)cc23)cc(Cl)c1CN1CCC(N(C)C(=O)C2CCN(c3ccc(NC4CCC(=O)NC4=O)cc3F)CC2)CC1 ; CN(C(=O)C1CCN(c2ccc(NC3CCC(=O)NC3=O)cc2F)CC1)C1CCN(Cc2ccc(-c3cn(C)c(=O)c4ccc(F)cc34)cc2OC(F)(F)F)CC1 ; COc1cc(-c2cn(C)c(=O)c3ccc(F)cc23)cc(F)c1CC1CCN(CC(=O)C2CCN(c3ccc(NC4CCC(=O)NC4=O)cc3F)CC2)CC1]

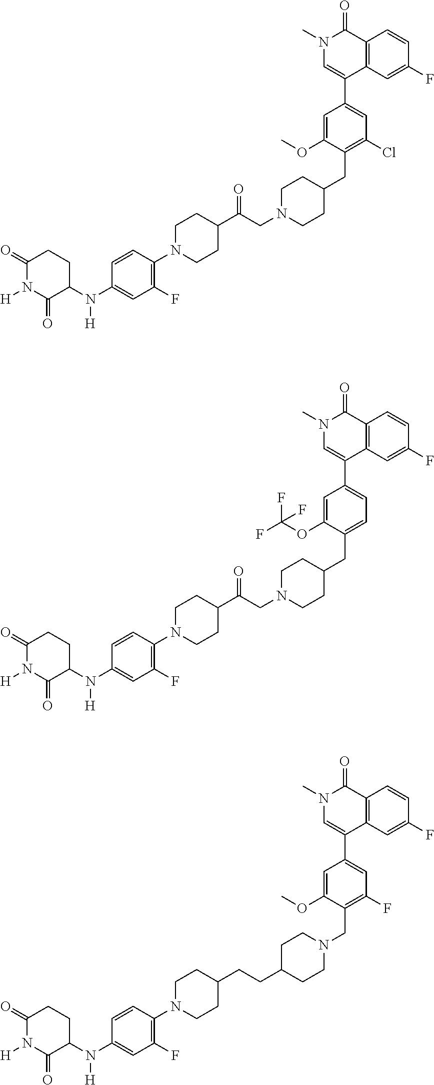 [SMILES: COc1cc(-c2cn(C)c(=O)c3ccc(F)cc23)cc(Cl)c1CC1CCN(CC(=O)C2CCN(c3ccc(NC4CCC(=O)NC4=O)cc3F)CC2)CC1 ; Cn1cc(-c2ccc(CC3CCN(CC(=O)C4CCN(c5ccc(NC6CCC(=O)NC6=O)cc5F)CC4)CC3)c(OC(F)(F)F)c2)c2cc(F)ccc2c1=O ; COc1cc(-c2cn(C)c(=O)c3ccc(F)cc23)cc(F)c1CN1CCC(CCC2CCN(c3ccc(NC4CCC(=O)NC4=O)cc3F)CC2)CC1]

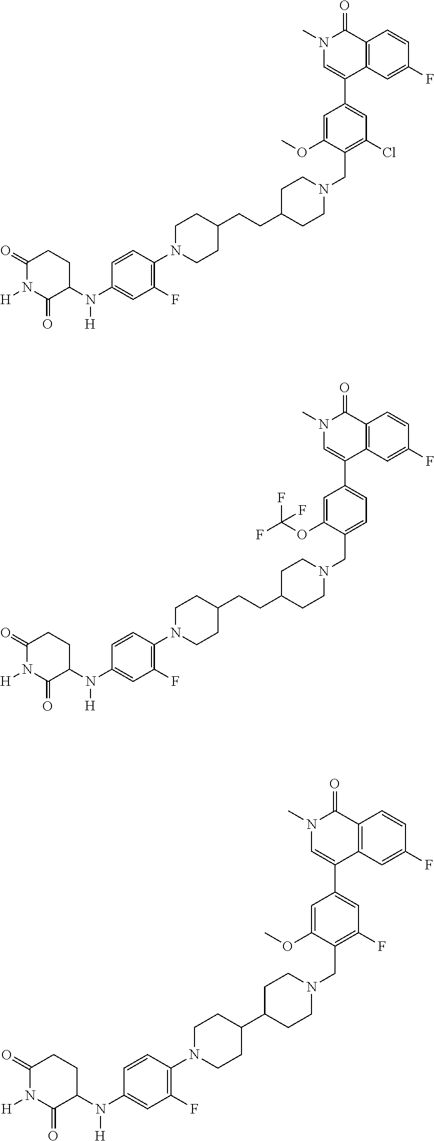 [SMILES: COc1cc(-c2cn(C)c(=O)c3ccc(F)cc23)cc(Cl)c1CN1CCC(CCC2CCN(c3ccc(NC4CCC(=O)NC4=O)cc3F)CC2)CC1 ; Cn1cc(-c2ccc(CN3CCC(CCC4CCN(c5ccc(NC6CCC(=O)NC6=O)cc5F)CC4)CC3)c(OC(F)(F)F)c2)c2cc(F)ccc2c1=O ; COc1cc(-c2cn(C)c(=O)c3ccc(F)cc23)cc(F)c1CN1CCC(C2CCN(c3ccc(NC4CCC(=O)NC4=O)cc3F)CC2)CC1]

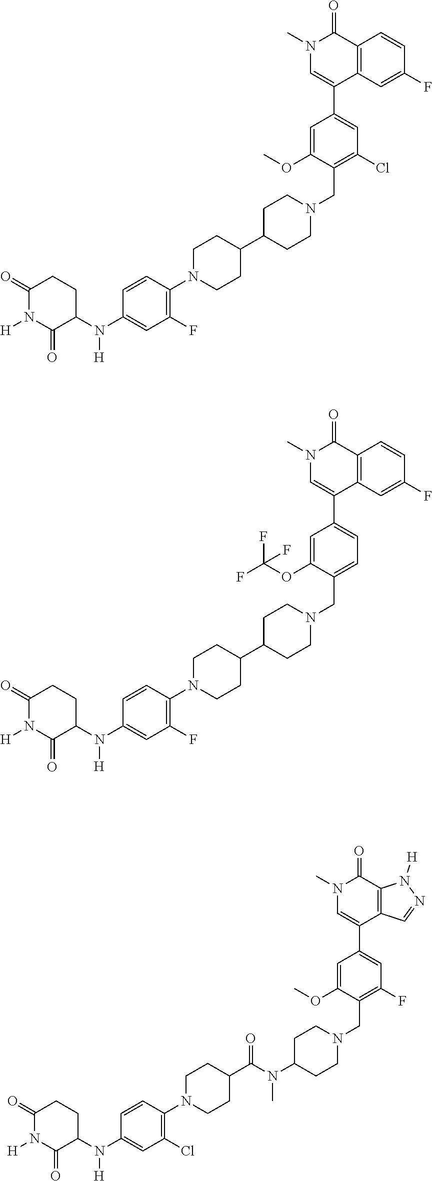 [SMILES: COc1cc(-c2cn(C)c(=O)c3ccc(F)cc23)cc(Cl)c1CN1CCC(C2CCN(c3ccc(NC4CCC(=O)NC4=O)cc3F)CC2)CC1 ; Cn1cc(-c2ccc(CN3CCC(C4CCN(c5ccc(NC6CCC(=O)NC6=O)cc5F)CC4)CC3)c(OC(F)(F)F)c2)c2cc(F)ccc2c1=O ; COc1cc(-c2cn(C)c(=O)c3[nH]ncc23)cc(F)c1CN1CCC(N(C)C(=O)C2CCN(c3ccc(NC4CCC(=O)NC4=O)cc3Cl)CC2)CC1]

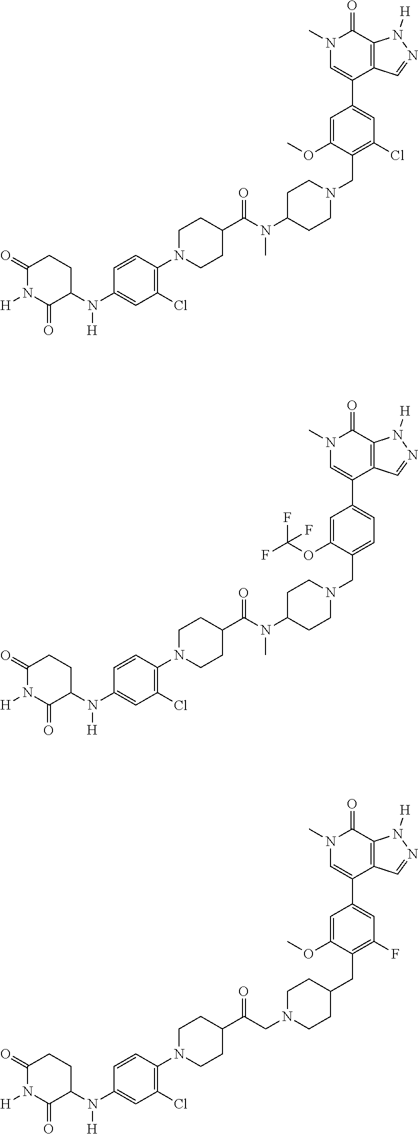 [SMILES: COc1cc(-c2cn(C)c(=O)c3[nH]ncc23)cc(Cl)c1CN1CCC(N(C)C(=O)C2CCN(c3ccc(NC4CCC(=O)NC4=O)cc3Cl)CC2)CC1 ; CN(C(=O)C1CCN(c2ccc(NC3CCC(=O)NC3=O)cc2Cl)CC1)C1CCN(Cc2ccc(-c3cn(C)c(=O)c4[nH]ncc34)cc2OC(F)(F)F)CC1 ; COc1cc(-c2cn(C)c(=O)c3[nH]ncc23)cc(F)c1CC1CCN(CC(=O)C2CCN(c3ccc(NC4CCC(=O)NC4=O)cc3Cl)CC2)CC1]

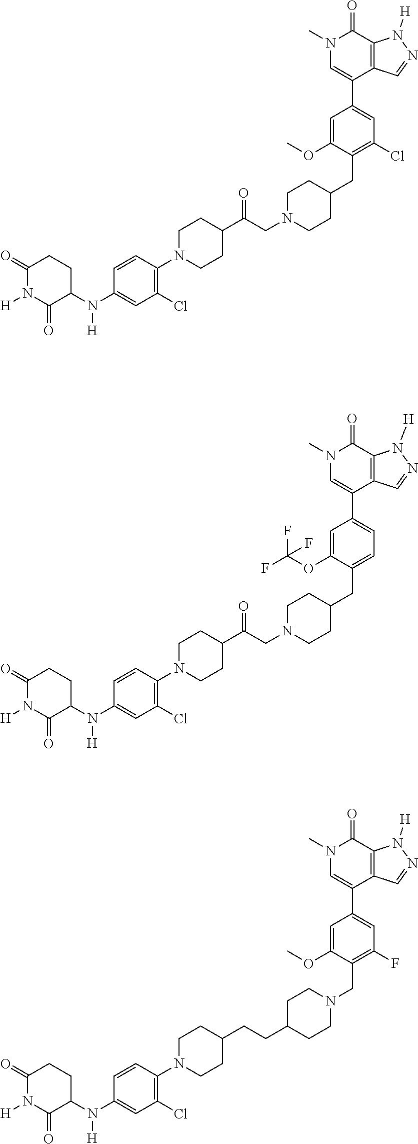 [SMILES: COc1cc(-c2cn(C)c(=O)c3[nH]ncc23)cc(Cl)c1CC1CCN(CC(=O)C2CCN(c3ccc(NC4CCC(=O)NC4=O)cc3Cl)CC2)CC1 ; Cn1cc(-c2ccc(CC3CCN(CC(=O)C4CCN(c5ccc(NC6CCC(=O)NC6=O)cc5Cl)CC4)CC3)c(OC(F)(F)F)c2)c2cn[nH]c2c1=O ; COc1cc(-c2cn(C)c(=O)c3[nH]ncc23)cc(F)c1CN1CCC(CCC2CCN(c3ccc(NC4CCC(=O)NC4=O)cc3Cl)CC2)CC1]

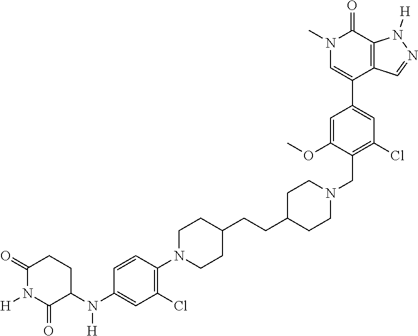 [SMILES: COc1cc(-c2cn(C)c(=O)c3[nH]ncc23)cc(Cl)c1CN1CCC(CCC2CCN(c3ccc(NC4CCC(=O)NC4=O)cc3Cl)CC2)CC1]

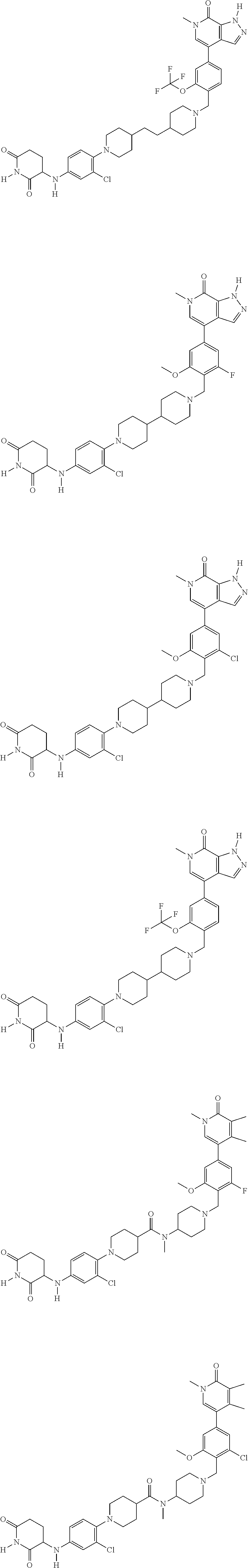

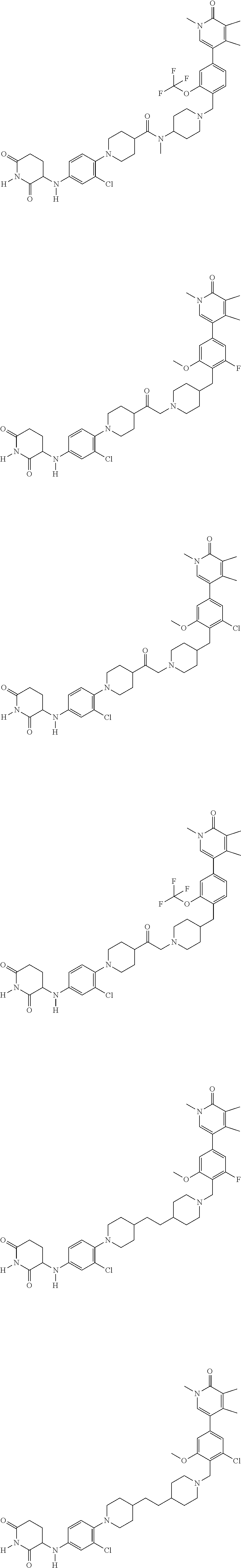

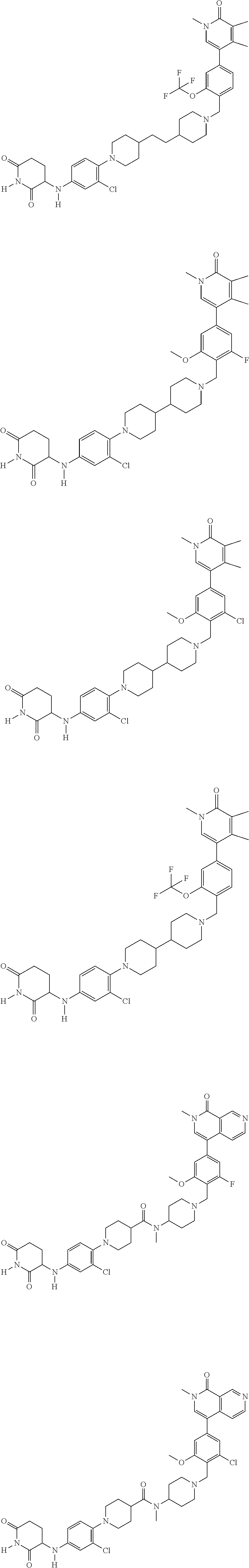

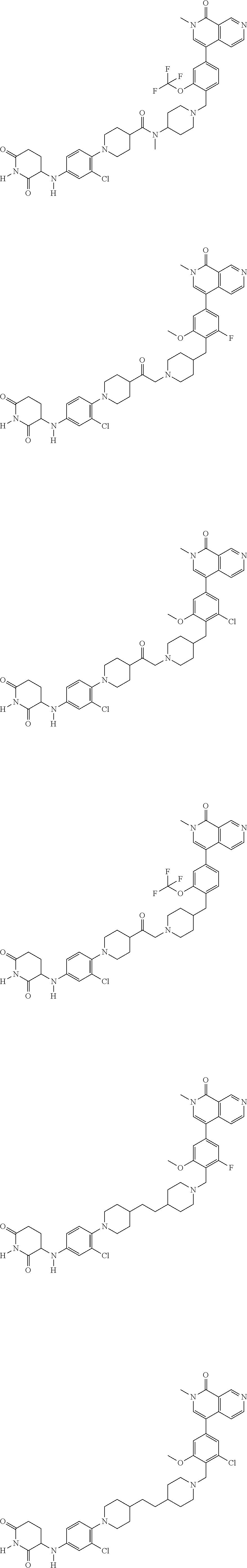

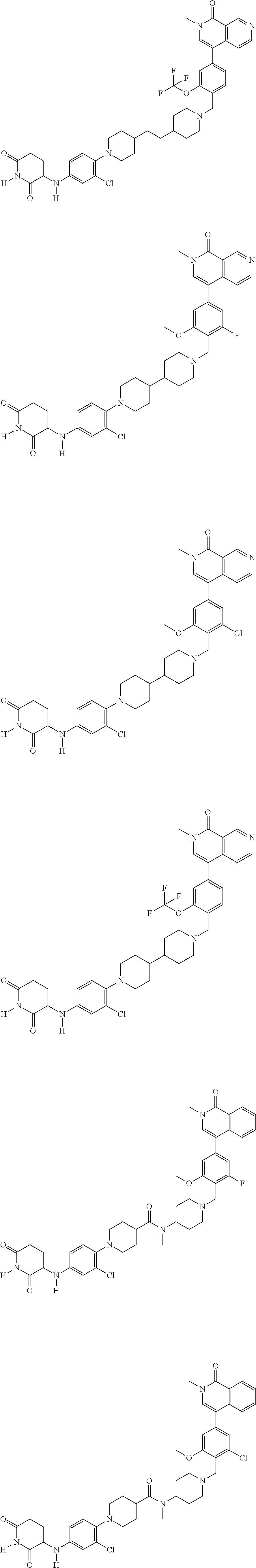

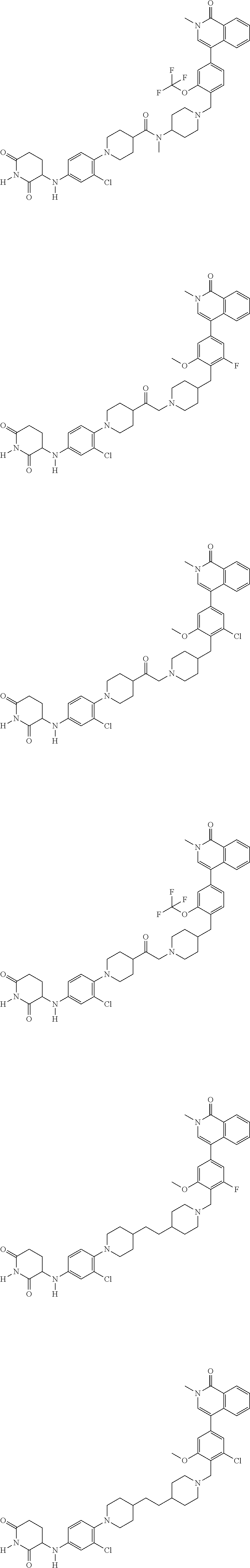

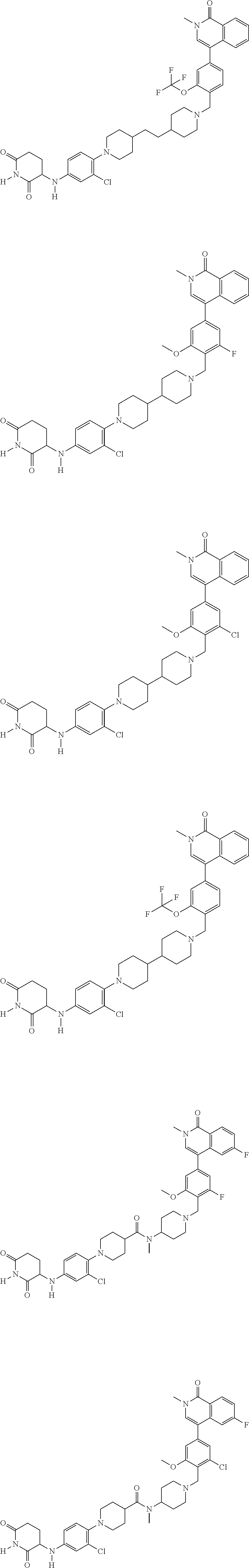

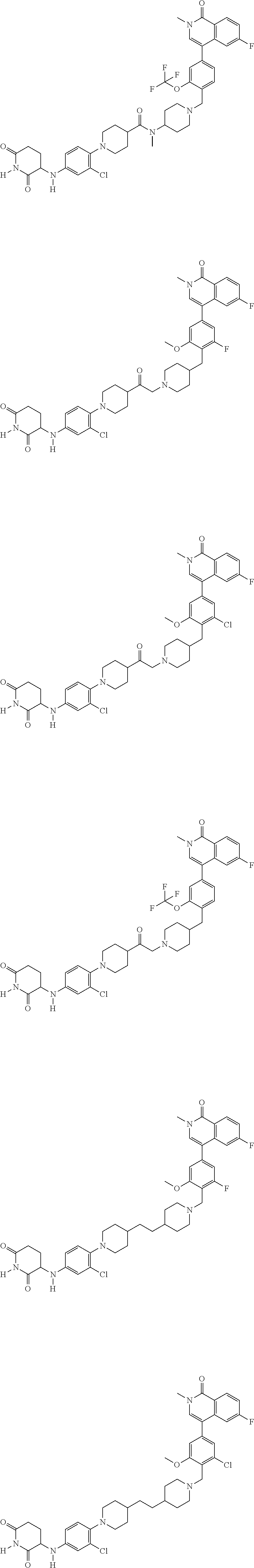

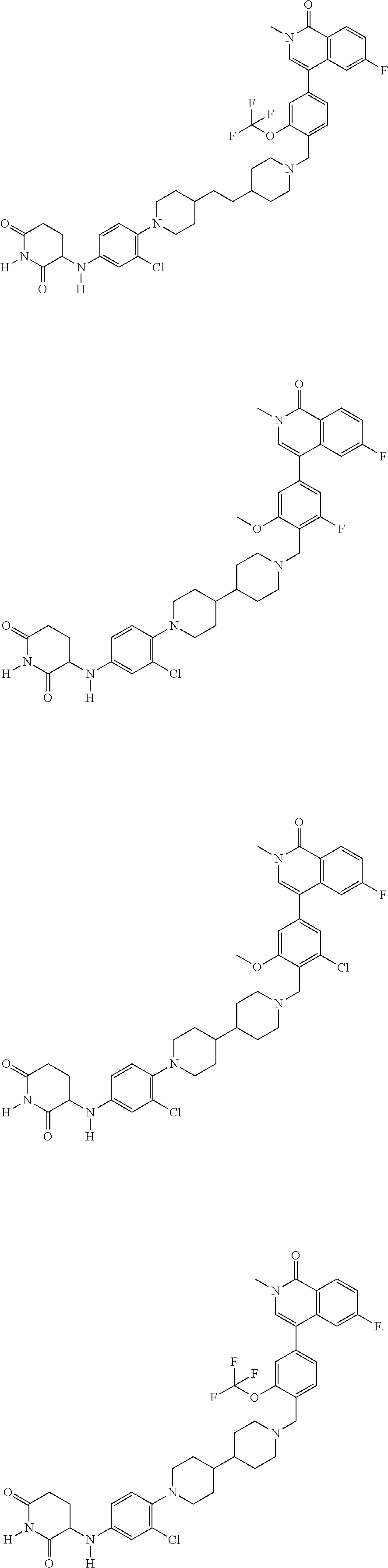 [SMILES: Cn1cc(-c2ccc(CN3CCC(CCC4CCN(c5ccc(NC6CCC(=O)NC6=O)cc5Cl)CC4)CC3)c(OC(F)(F)F)c2)c2cc(F)ccc2c1=O ; COc1cc(-c2cn(C)c(=O)c3ccc(F)cc23)cc(F)c1CN1CCC(C2CCN(c3ccc(NC4CCC(=O)NC4=O)cc3Cl)CC2)CC1 ; COc1cc(-c2cn(C)c(=O)c3ccc(F)cc23)cc(Cl)c1CN1CCC(C2CCN(c3ccc(NC4CCC(=O)NC4=O)cc3Cl)CC2)CC1 ; Cn1cc(-c2ccc(CN3CCC(C4CCN(c5ccc(NC6CCC(=O)NC6=O)cc5Cl)CC4)CC3)c(OC(F)(F)F)c2)c2cc(F)ccc2c1=O]

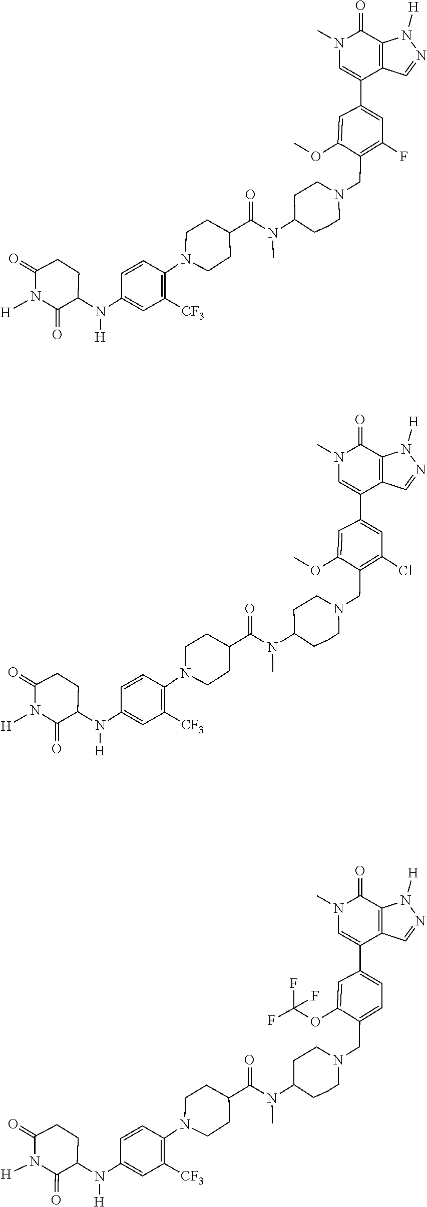 [SMILES: COC1=CC(c2cn(C)c(=O)c3[nH]ncc23)=CC(F)=C(CN2CCCC(N(C)C(=O)CC3CCN(c4ccc(NC5CCC(=O)NC5=O)cc4C(F)(F)F)CC3)CC2)C1 ; COC1=CC(c2cn(C)c(=O)c3[nH]ncc23)=CC(Cl)=C(CN2CCCC(N(C)C(=O)CC3CCN(c4ccc(NC5CCC(=O)NC5=O)cc4C(F)(F)F)CC3)CC2)C1 ; CN(C(=O)CC1CCN(c2ccc(NC3CCC(=O)NC3=O)cc2C(F)(F)F)CC1)C1CCCN(CC2=CC=C(c3cn(C)c(=O)c4[nH]ncc34)C=C(OC(F)(F)F)C2)CC1]

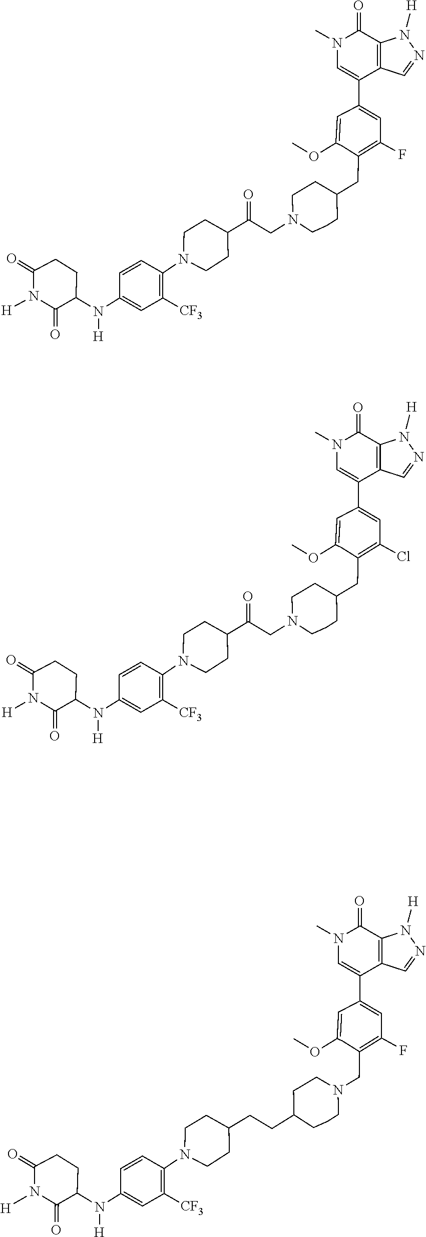 [SMILES: COC1=CC(c2cn(C)c(=O)c3[nH]ncc23)=CC(F)=C(CC2CCCN(CC(=O)CC3CCN(c4ccc(NC5CCC(=O)NC5=O)cc4C(F)(F)F)CC3)CC2)C1 ; COC1=CC(c2cn(C)c(=O)c3[nH]ncc23)=CC(Cl)=C(CC2CCCN(CC(=O)CC3CCN(c4ccc(NC5CCC(=O)NC5=O)cc4C(F)(F)F)CC3)CC2)C1 ; COC1=CC(c2cn(C)c(=O)c3[nH]ncc23)=CC(F)=C(CN2CCCC(CCCC3CCN(c4ccc(NC5CCC(=O)NC5=O)cc4C(F)(F)F)CC3)CC2)C1]

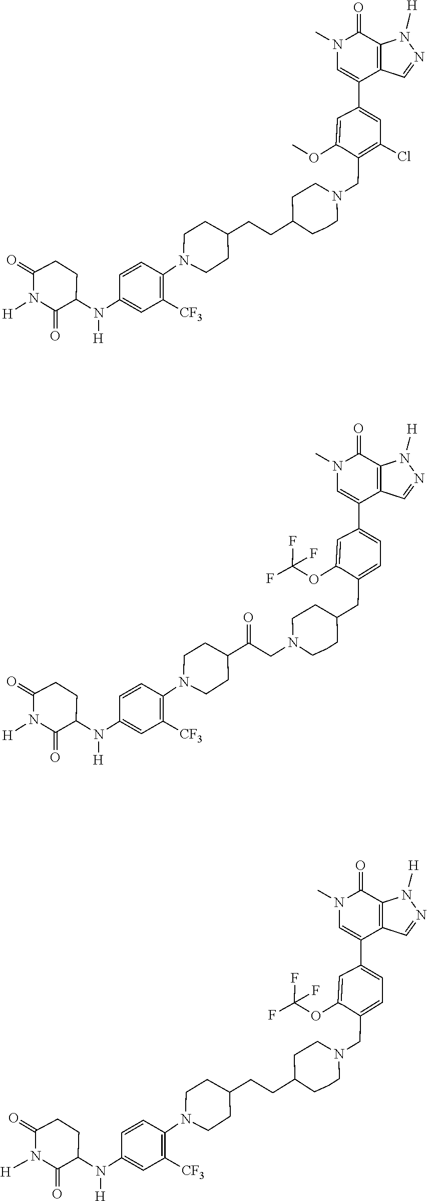 [SMILES: COC1=CC(c2cn(C)c(=O)c3[nH]ncc23)=CC(Cl)=C(CN2CCCC(CCCC3CCN(c4ccc(NC5CCC(=O)NC5=O)cc4C(F)(F)F)CC3)CC2)C1 ; Cn1cc(C2=CC=C(CC3CCCN(CC(=O)CC4CCN(c5ccc(NC6CCC(=O)NC6=O)cc5C(F)(F)F)CC4)CC3)CC(OC(F)(F)F)=C2)c2cn[nH]c2c1=O ; Cn1cc(C2=CC=C(CN3CCCC(CCCC4CCN(c5ccc(NC6CCC(=O)NC6=O)cc5C(F)(F)F)CC4)CC3)CC(OC(F)(F)F)=C2)c2cn[nH]c2c1=O]

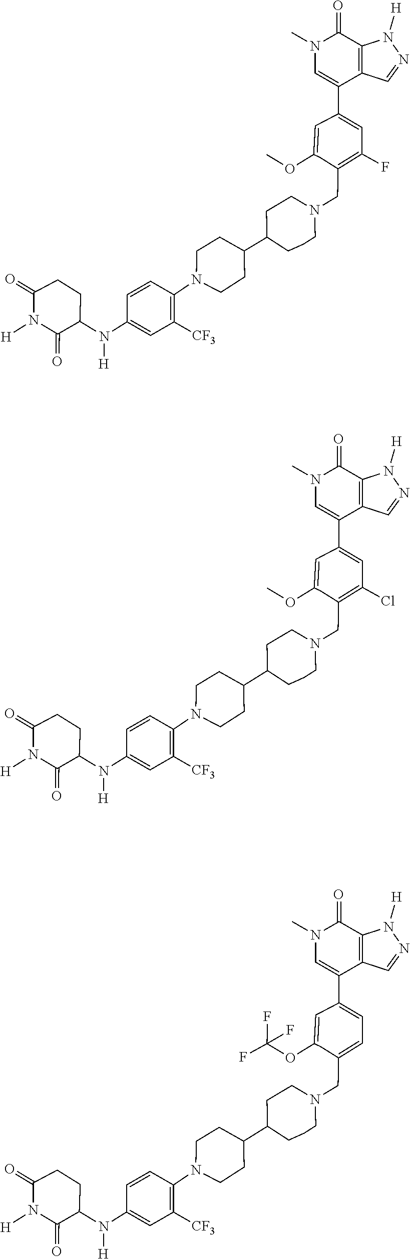 [SMILES: COC1=CC(c2cn(C)c(=O)c3[nH]ncc23)=CC(F)=C(CN2CCCC(CC3CCN(c4ccc(NC5CCC(=O)NC5=O)cc4C(F)(F)F)CC3)CC2)C1 ; COC1=CC(c2cn(C)c(=O)c3[nH]ncc23)=CC(Cl)=C(CN2CCCC(C3CCN(c4ccc(NC5CCC(=O)NC5=O)cc4C(F)(F)F)CC3)CC2)C1 ; Cn1cc(C2=CC=C(CN3CCCC(C4CCN(c5ccc(NC6CCC(=O)NC6=O)cc5C(F)(F)F)CC4)CC3)CC(OC(F)(F)F)=C2)c2cn[nH]c2c1=O]

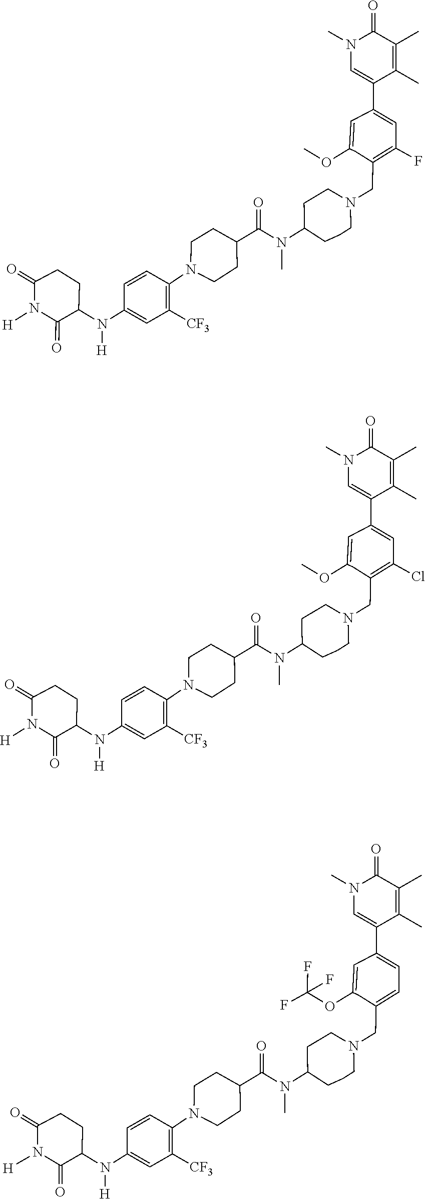 [SMILES: COC1=CC(c2cn(C)c(=O)c(C)c2C)=CC(F)=C(CN2CCCC(N(C)C(=O)CC3CCN(c4ccc(NC5CCC(=O)NC5=O)cc4C(F)(F)F)CC3)CC2)C1 ; COC1=CC(c2cn(C)c(=O)c(C)c2C)=CC(Cl)=C(CN2CCCC(N(C)C(=O)CC3CCN(c4ccc(NC5CCC(=O)NC5=O)cc4C(F)(F)F)CC3)CC2)C1 ; Cc1c(C2=CC=C(CN3CCCC(N(C)C(=O)CC4CCN(c5ccc(NC6CCC(=O)NC6=O)cc5C(F)(F)F)CC4)CC3)CC(OC(F)(F)F)=C2)cn(C)c(=O)c1C]

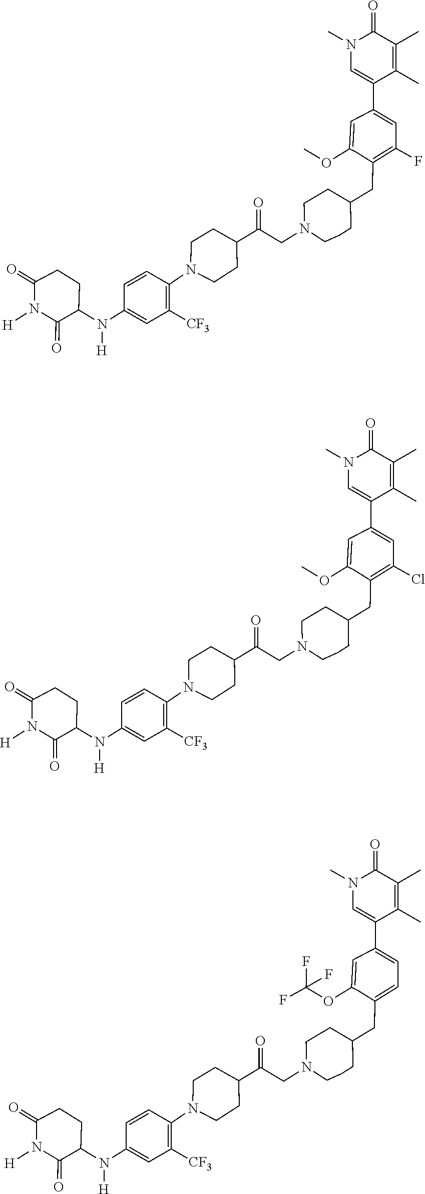 [SMILES: COC1=CC(c2cn(C)c(=O)c(C)c2C)=CC(F)=C(CC2CCCN(CC(=O)CC3CCN(c4ccc(NC5CCC(=O)NC5=O)cc4C(F)(F)F)CC3)CC2)C1 ; COC1=CC(c2cn(C)c(=O)c(C)c2C)=CC(Cl)=C(CC2CCCN(CC(=O)CC3CCN(c4ccc(NC5CCC(=O)NC5=O)cc4C(F)(F)F)CC3)CC2)C1 ; Cc1c(C2=CC=C(CC3CCCN(CC(=O)CC4CCN(c5ccc(NC6CCC(=O)NC6=O)cc5C(F)(F)F)CC4)CC3)CC(OC(F)(F)F)=C2)cn(C)c(=O)c1C]

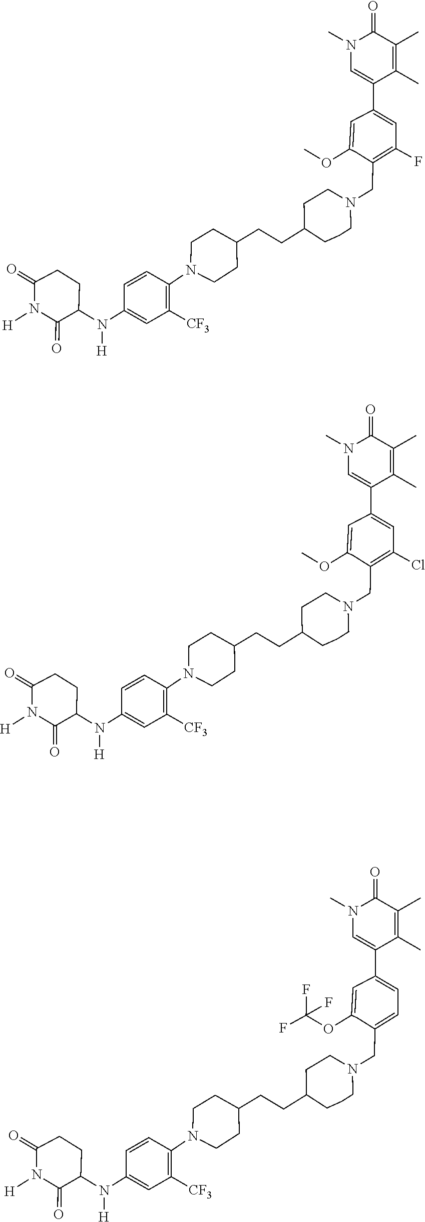 [SMILES: COC1=CC(c2cn(C)c(=O)c(C)c2C)=CC(F)=C(CN2CCCC(CCCC3CCN(c4ccc(NC5CCC(=O)NC5=O)cc4C(F)(F)F)CC3)CC2)C1 ; COC1=CC(c2cn(C)c(=O)c(C)c2C)=CC(Cl)=C(CN2CCCC(CCCC3CCN(c4ccc(NC5CCC(=O)NC5=O)cc4C(F)(F)F)CC3)CC2)C1 ; Cc1c(C2=CC=C(CN3CCCC(CCCC4CCN(c5ccc(NC6CCC(=O)NC6=O)cc5C(F)(F)F)CC4)CC3)CC(OC(F)(F)F)=C2)cn(C)c(=O)c1C]

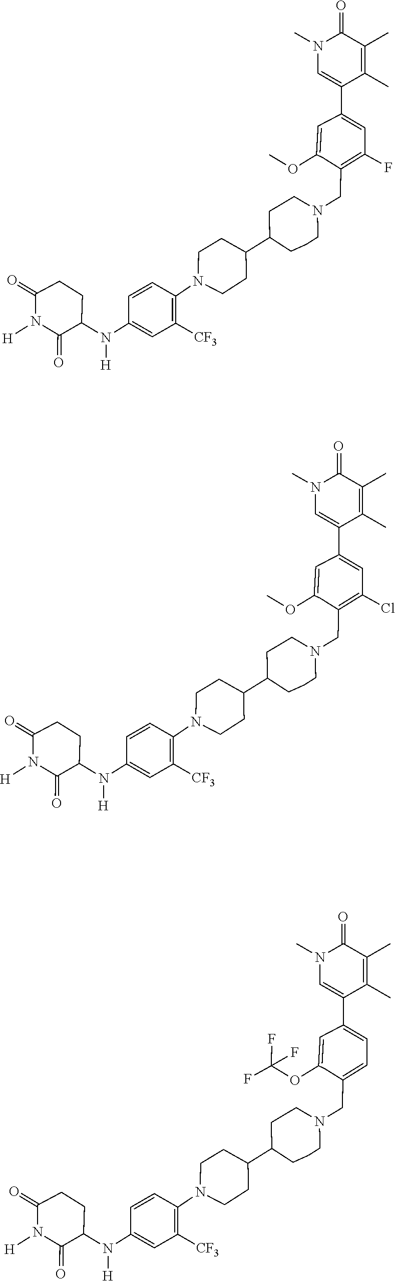 [SMILES: COC1=CC(c2cn(C)c(=O)c(C)c2C)=CC(F)=C(CN2CCCC(C3CCN(c4ccc(NC5CCC(=O)NC5=O)cc4C(F)(F)F)CC3)CC2)C1 ; COC1=CC(c2cn(C)c(=O)c(C)c2C)=CC(Cl)=C(CN2CCCC(C3CCN(c4ccc(NC5CCC(=O)NC5=O)cc4C(F)(F)F)CC3)CC2)C1 ; Cc1c(C2=CC=C(CN3CCCC(C4CCN(c5ccc(NC6CCC(=O)NC6=O)cc5C(F)(F)F)CC4)CC3)CC(OC(F)(F)F)=C2)cn(C)c(=O)c1C]

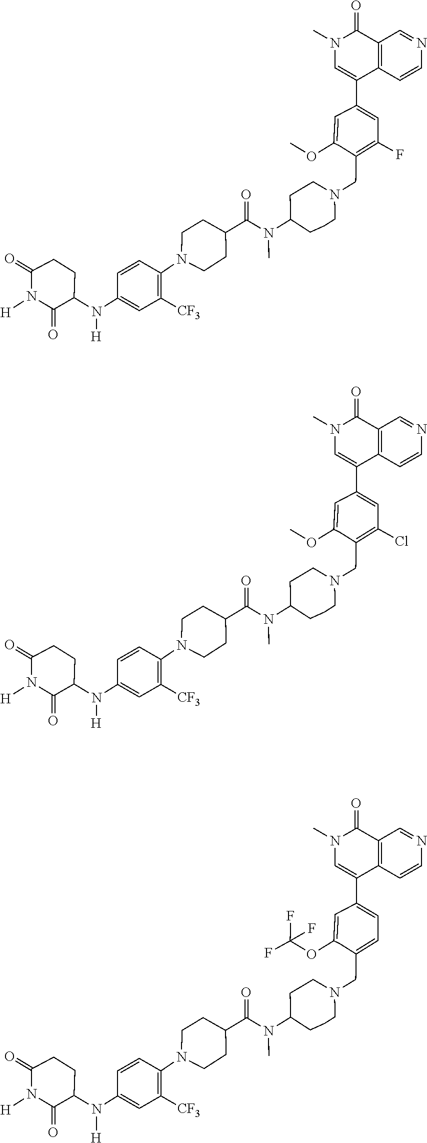 [SMILES: COC1=CC(c2cn(C)c(=O)c3cnccc23)=CC(F)=C(CN2CCCC(N(C)C(=O)CC3CCN(c4ccc(NC5CCC(=O)NC5=O)cc4C(F)(F)F)CC3)CC2)C1 ; COC1=CC(c2cn(C)c(=O)c3cnccc23)=CC(Cl)=C(CN2CCCC(N(C)C(=O)CC3CCN(c4ccc(NC5CCC(=O)NC5=O)cc4C(F)(F)F)CC3)CC2)C1 ; CN(C(=O)CC1CCN(c2ccc(NC3CCC(=O)NC3=O)cc2C(F)(F)F)CC1)C1CCCN(CC2=CC=C(c3cn(C)c(=O)c4cnccc34)C=C(OC(F)(F)F)C2)CC1]

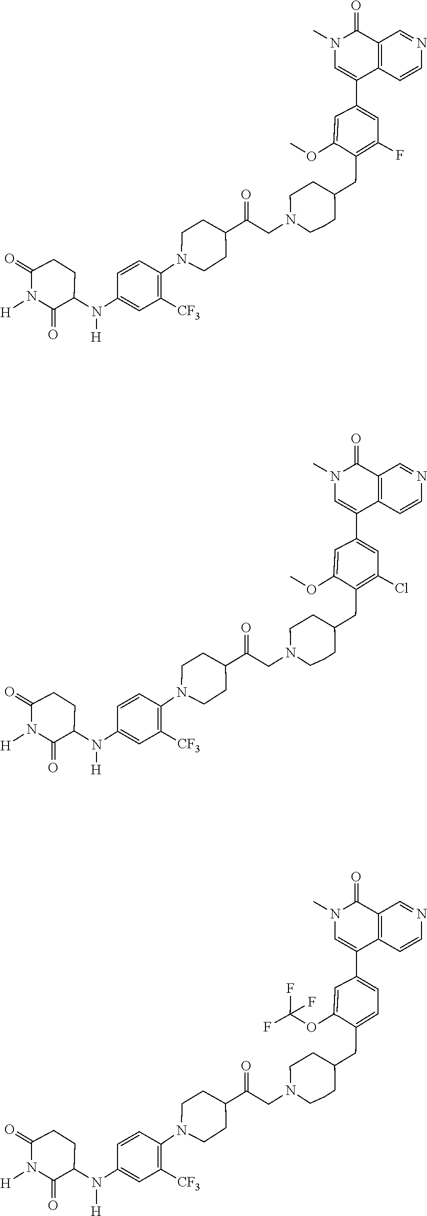 [SMILES: COC1=CC(c2cn(C)c(=O)c3cnccc23)=CC(F)=C(CC2CCCN(CC(=O)CC3CCN(c4ccc(NC5CCC(=O)NC5=O)cc4C(F)(F)F)CC3)CC2)C1 ; COC1=CC(c2cn(C)c(=O)c3cnccc23)=CC(Cl)=C(CC2CCCN(CC(=O)CC3CCN(c4ccc(NC5CCC(=O)NC5=O)cc4C(F)(F)F)CC3)CC2)C1 ; Cn1cc(C2=CC=C(CC3CCCN(CC(=O)CC4CCN(c5ccc(NC6CCC(=O)NC6=O)cc5C(F)(F)F)CC4)CC3)CC(OC(F)(F)F)=C2)c2ccncc2c1=O]

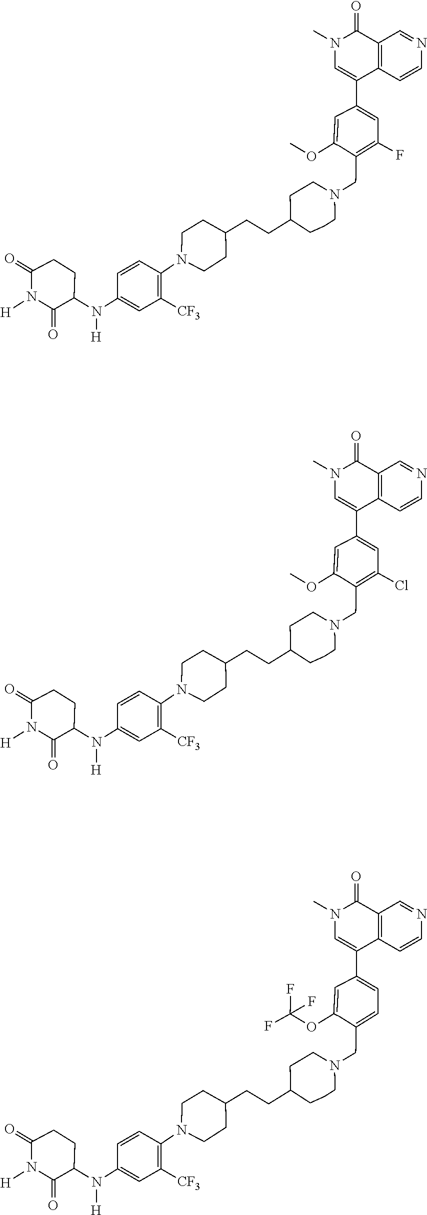 [SMILES: COC1=CC(c2cn(C)c(=O)c3cnccc23)=CC(F)=C(CN2CCCC(CCCC3CCN(c4ccc(NC5CCC(=O)NC5=O)cc4C(F)(F)F)CC3)CC2)C1 ; COC1=CC(c2cn(C)c(=O)c3cnccc23)=CC(Cl)=C(CN2CCCC(CCCC3CCN(c4ccc(NC5CCC(=O)NC5=O)cc4C(F)(F)F)CC3)CC2)C1 ; Cn1cc(C2=CC=C(CN3CCCC(CCCC4CCN(c5ccc(NC6CCC(=O)NC6=O)cc5C(F)(F)F)CC4)CC3)CC(OC(F)(F)F)=C2)c2ccncc2c1=O]

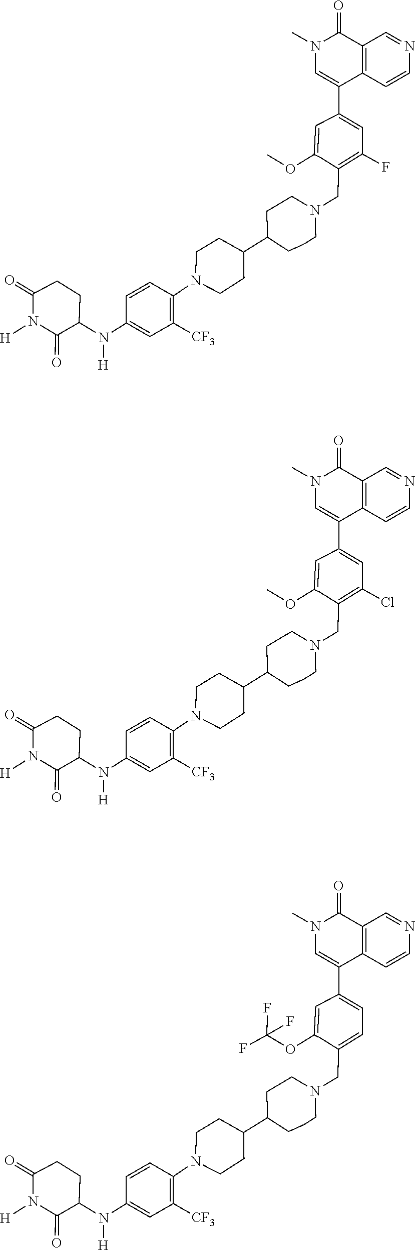 [SMILES: COC1=CC(c2cn(C)c(=O)c3cnccc23)=CC(F)=C(CN2CCCC(C3CCN(c4ccc(NC5CCC(=O)NC5=O)cc4C(F)(F)F)CC3)CC2)C1 ; COC1=CC(c2cn(C)c(=O)c3cnccc23)=CC(Cl)=C(CN2CCCC(C3CCN(c4ccc(NC5CCC(=O)NC5=O)cc4C(F)(F)F)CC3)CC2)C1 ; Cn1cc(C2=CC=C(CN3CCCC(C4CCN(c5ccc(NC6CCC(=O)NC6=O)cc5C(F)(F)F)CC4)CC3)CC(OC(F)(F)F)=C2)c2ccncc2c1=O]

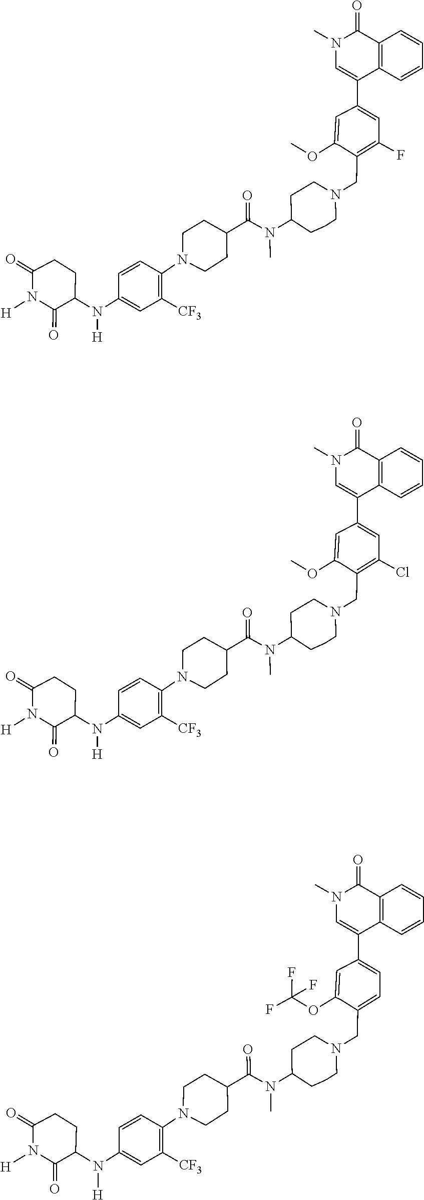 [SMILES: COC1=CC(c2cn(C)c(=O)c3ccccc23)=CC(F)=C(CN2CCCC(N(C)C(=O)C3CCN(c4ccc(NC5CCC(=O)NC5=O)cc4C(F)(F)F)CC3)CC2)C1 ; COC1=CC(c2cn(C)c(=O)c3ccccc23)=CC(Cl)=C(CN2CCCC(N(C)C(=O)C3CCN(c4ccc(NC5CCC(=O)NC5=O)cc4C(F)(F)F)CC3)CC2)C1 ; CN(C(=O)C1CCN(c2ccc(NC3CCC(=O)NC3=O)cc2C(F)(F)F)CC1)C1CCCN(CC2=CC=C(c3cn(C)c(=O)c4ccccc34)C=C(OC(F)(F)F)C2)CC1]

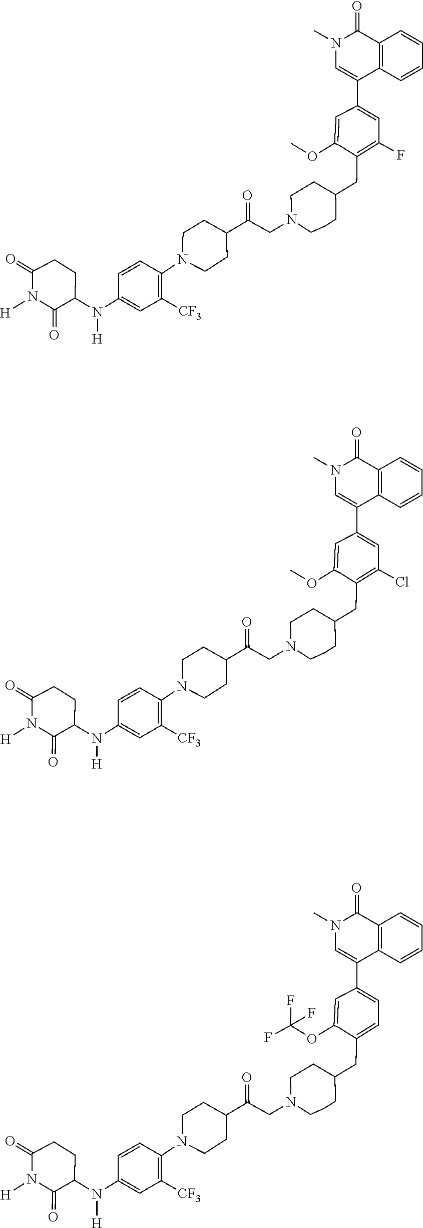 [SMILES: COC1=CC(c2cn(C)c(=O)c3ccccc23)=CC(F)=C(CC2CCCN(CC(=O)C3CCN(c4ccc(NC5CCC(=O)NC5=O)cc4C(F)(F)F)CC3)CC2)C1 ; COC1=CC(c2cn(C)c(=O)c3ccccc23)=CC(Cl)=C(CC2CCCN(CC(=O)C3CCN(c4ccc(NC5CCC(=O)NC5=O)cc4C(F)(F)F)CC3)CC2)C1 ; Cn1cc(C2=CC=C(CC3CCCN(CC(=O)C4CCN(c5ccc(NC6CCC(=O)NC6=O)cc5C(F)(F)F)CC4)CC3)CC(OC(F)(F)F)=C2)c2ccccc2c1=O]

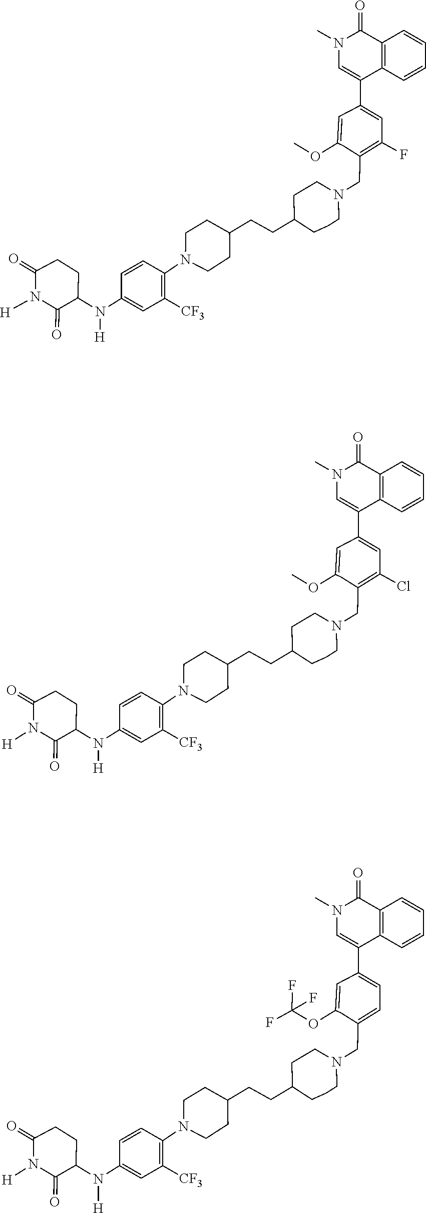 [SMILES: COC1=CC(c2cn(C)c(=O)c3ccccc23)=CC(F)=C(CN2CCCC(CCC3CCN(c4ccc(NC5CCC(=O)NC5=O)cc4C(F)(F)F)CC3)CC2)C1 ; COC1=CC(c2cn(C)c(=O)c3ccccc23)=CC(Cl)=C(CN2CCCC(CCC3CCN(c4ccc(NC5CCC(=O)NC5=O)cc4C(F)(F)F)CC3)CC2)C1 ; Cn1cc(C2=CC=C(CN3CCCC(CCC4CCN(c5ccc(NC6CCC(=O)NC6=O)cc5C(F)(F)F)CC4)CC3)CC(OC(F)(F)F)=C2)c2ccccc2c1=O]

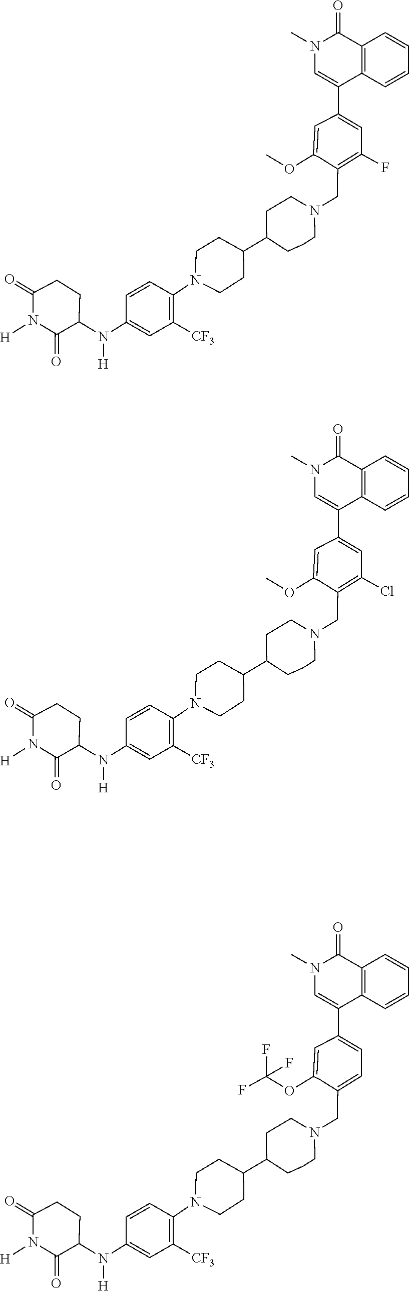 [SMILES: COC1=CC(c2cn(C)c(=O)c3ccccc23)=CC(F)=C(CN2CCCC(C3CCN(c4ccc(NC5CCC(=O)NC5=O)cc4C(F)(F)F)CC3)CC2)C1 ; COC1=CC(c2cn(C)c(=O)c3ccccc23)=CC(Cl)=C(CN2CCCC(C3CCN(c4ccc(NC5CCC(=O)NC5=O)cc4C(F)(F)F)CC3)CC2)C1 ; Cn1cc(C2=CC=C(CN3CCCC(C4CCN(c5ccc(NC6CCC(=O)NC6=O)cc5C(F)(F)F)CC4)CC3)CC(OC(F)(F)F)=C2)c2ccccc2c1=O]

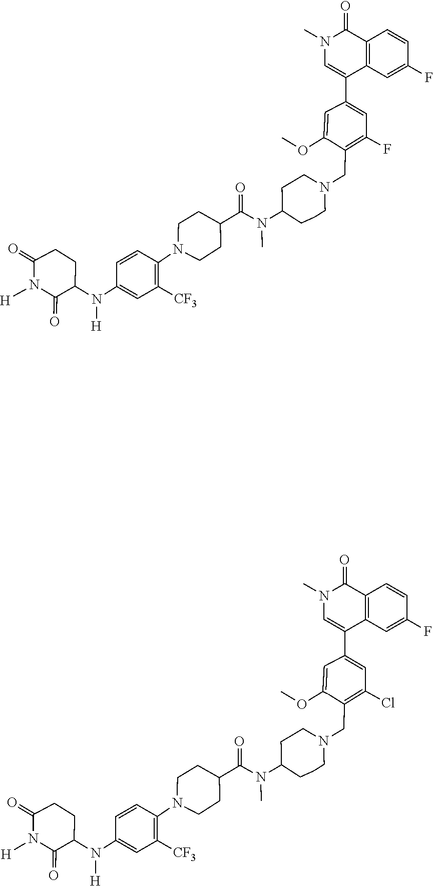 [SMILES: COC1=CC(c2cn(C)c(=O)c3ccc(F)cc23)=CC(F)=C(CN2CCCC(N(C)C(=O)C3CCN(c4ccc(NC5CCC(=O)NC5=O)cc4C(F)(F)F)CC3)CC2)C1 ; COC1=CC(c2cn(C)c(=O)c3ccc(F)cc23)=CC(Cl)=C(CN2CCCC(N(C)C(=O)C3CCN(c4ccc(NC5CCC(=O)NC5=O)cc4C(F)(F)F)CC3)CC2)C1]

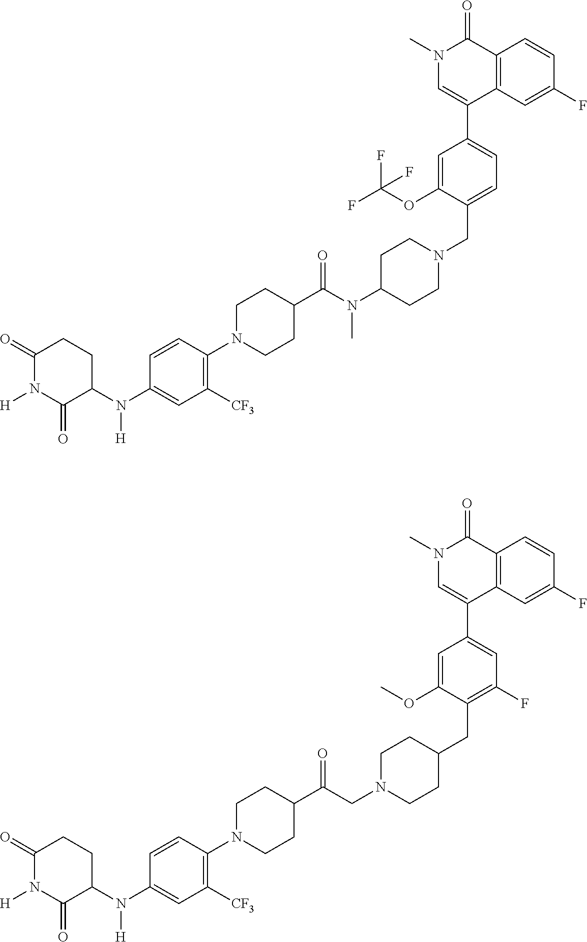 [SMILES: CN(C(=O)C1CCN(c2ccc(NC3CCC(=O)NC3=O)cc2C(F)(F)F)CC1)C1CCN(Cc2ccc(-c3cn(C)c(=O)c4ccc(F)cc34)cc2OC(F)(F)F)CC1 ; COc1cc(-c2cn(C)c(=O)c3ccc(F)cc23)cc(F)c1CC1CCN(CC(=O)C2CCN(c3ccc(NC4CCC(=O)NC4=O)cc3C(F)(F)F)CC2)CC1]

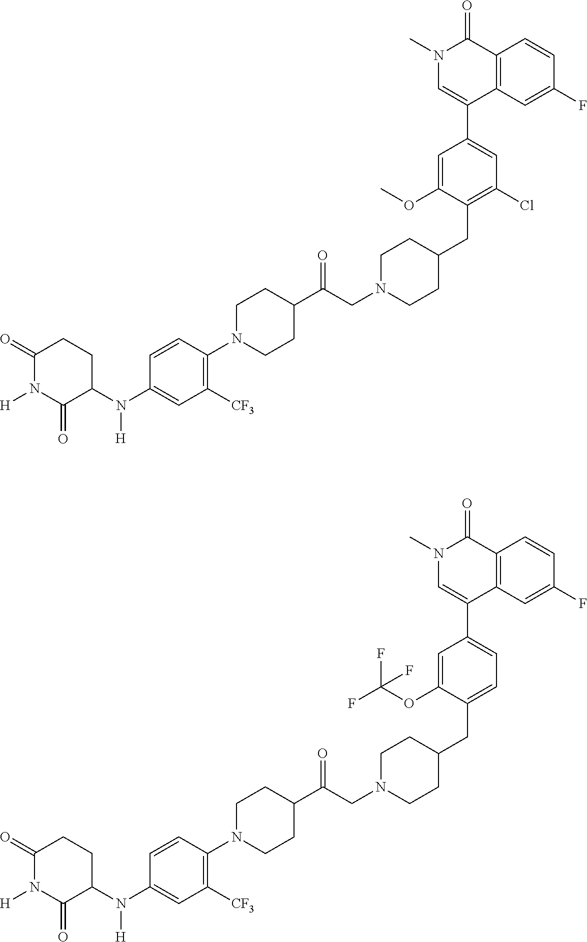 [SMILES: COc1cc(-c2cn(C)c(=O)c3ccc(F)cc23)cc(Cl)c1CC1CCN(CC(=O)C2CCN(c3ccc(NC4CCC(=O)NC4=O)cc3C(F)(F)F)CC2)CC1 ; Cn1cc(-c2ccc(CC3CCN(CC(=O)C4CCN(c5ccc(NC6CCC(=O)NC6=O)cc5C(F)(F)F)CC4)CC3)c(OC(F)(F)F)c2)c2cc(F)ccc2c1=O]

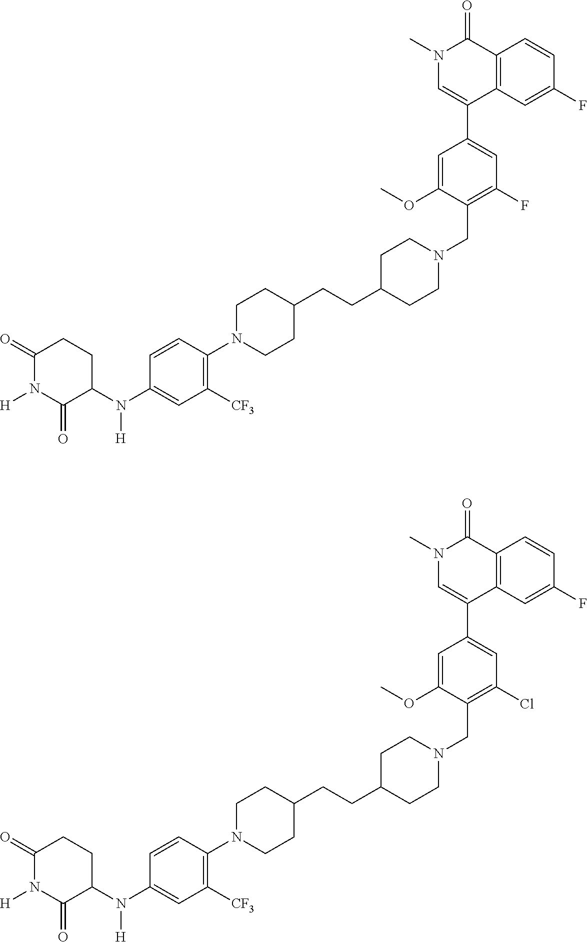 [SMILES: COc1cc(-c2cn(C)c(=O)c3ccc(F)cc23)cc(F)c1CN1CCC(CCC2CCN(c3ccc(NC4CCC(=O)NC4=O)cc3C(F)(F)F)CC2)CC1 ; COc1cc(-c2cn(C)c(=O)c3ccc(F)cc23)cc(Cl)c1CN1CCC(CCC2CCN(c3ccc(NC4CCC(=O)NC4=O)cc3C(F)(F)F)CC2)CC1]

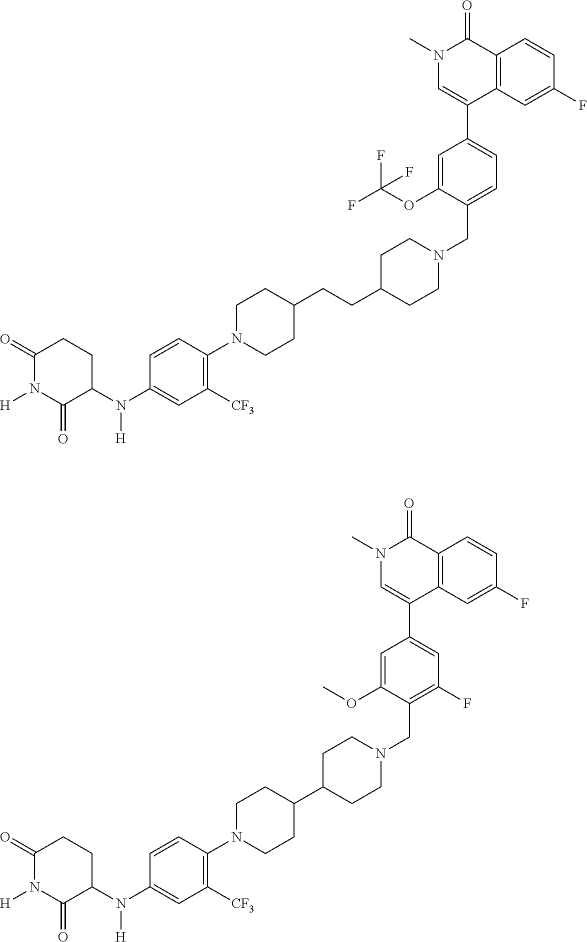 [SMILES: Cn1cc(-c2ccc(CN3CCC(CCC4CCN(c5ccc(NC6CCC(=O)NC6=O)cc5C(F)(F)F)CC4)CC3)c(OC(F)(F)F)c2)c2cc(F)ccc2c1=O ; COc1cc(-c2cn(C)c(=O)c3ccc(F)cc23)cc(F)c1CN1CCC(C2CCN(c3ccc(NC4CCC(=O)NC4=O)cc3C(F)(F)F)CC2)CC1]

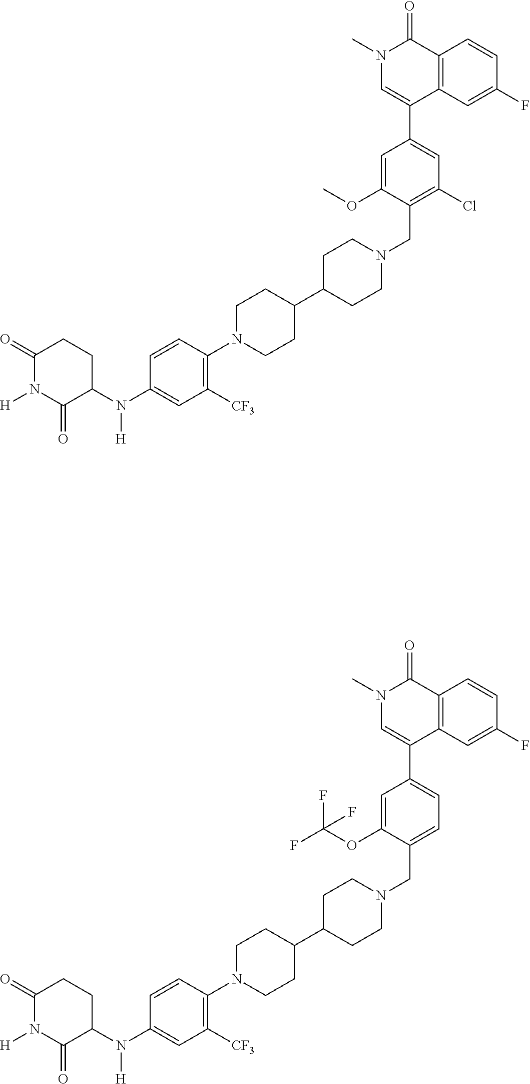 [SMILES: COc1cc(-c2cn(C)c(=O)c3ccc(F)cc23)cc(Cl)c1CN1CCC(C2CCN(c3ccc(NC4CCC(=O)NC4=O)cc3C(F)(F)F)CC2)CC1 ; Cn1cc(-c2ccc(CN3CCC(C4CCN(c5ccc(NC6CCC(=O)NC6=O)cc5C(F)(F)F)CC4)CC3)c(OC(F)(F)F)c2)c2cc(F)ccc2c1=O]

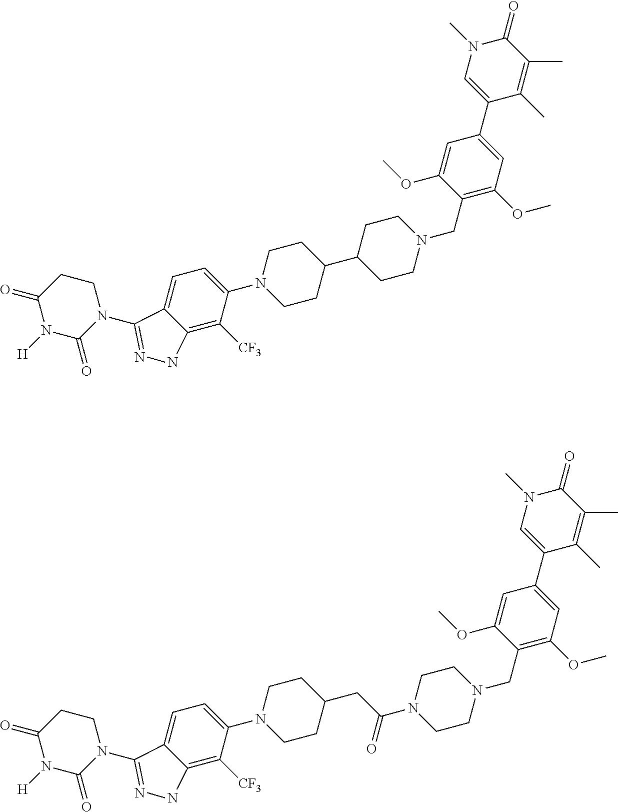 [SMILES: COc1cc(-c2cn(C)c(=O)c(C)c2C)cc(OC)c1CN1CCC(C2CCN(c3ccc4c(N5CCC(=O)NC5=O)n[nH]c4c3C(F)(F)F)CC2)CC1 ; COc1cc(-c2cn(C)c(=O)c(C)c2C)cc(OC)c1CN1CCN(C(=O)CC2CCN(c3ccc4c(N5CCC(=O)NC5=O)n[nH]c4c3C(F)(F)F)CC2)CC1]

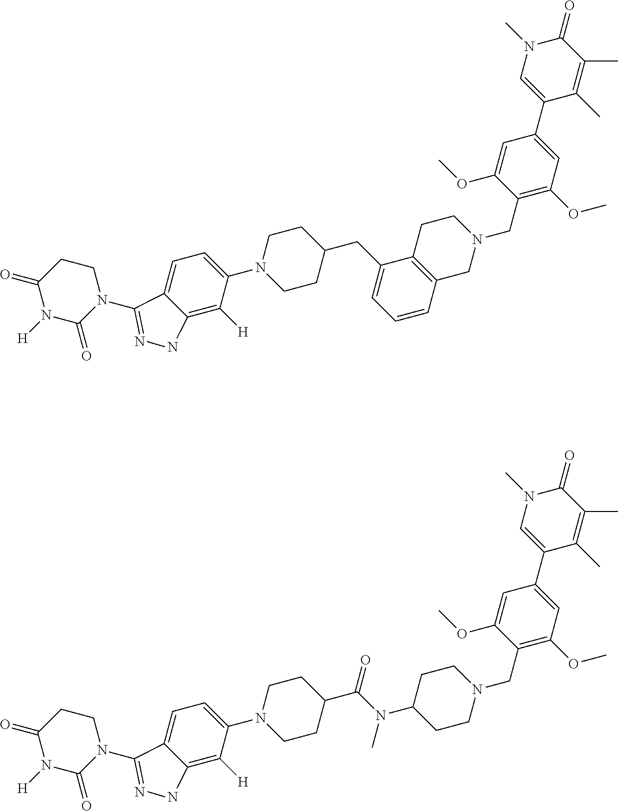 [SMILES: COc1cc(-c2cn(C)c(=O)c(C)c2C)cc(OC)c1CN1CCc2c(CC3CCN(c4ccc5c(N6CCC(=O)NC6=O)n[nH]c5c4)CC3)cccc2C1 ; COc1cc(-c2cn(C)c(=O)c(C)c2C)cc(OC)c1CN1CCC(N(C)C(=O)C2CCN(c3ccc4c(N5CCC(=O)NC5=O)n[nH]c4c3)CC2)CC1]

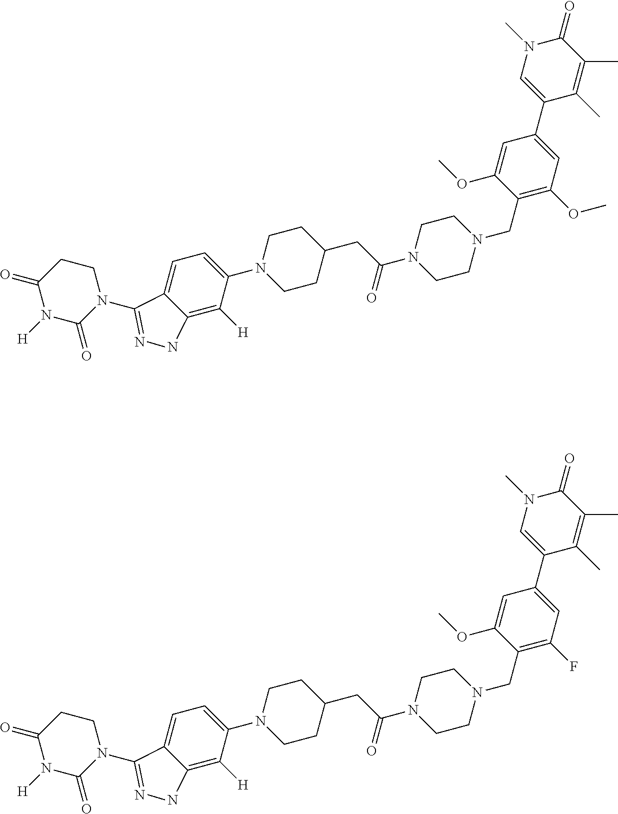 [SMILES: COc1cc(-c2cn(C)c(=O)c(C)c2C)cc(OC)c1CN1CCN(C(=O)CC2CCN(c3ccc4c(N5CCC(=O)NC5=O)n[nH]c4c3)CC2)CC1 ; COc1cc(-c2cn(C)c(=O)c(C)c2C)cc(F)c1CN1CCN(C(=O)CC2CCN(c3ccc4c(N5CCC(=O)NC5=O)n[nH]c4c3)CC2)CC1]

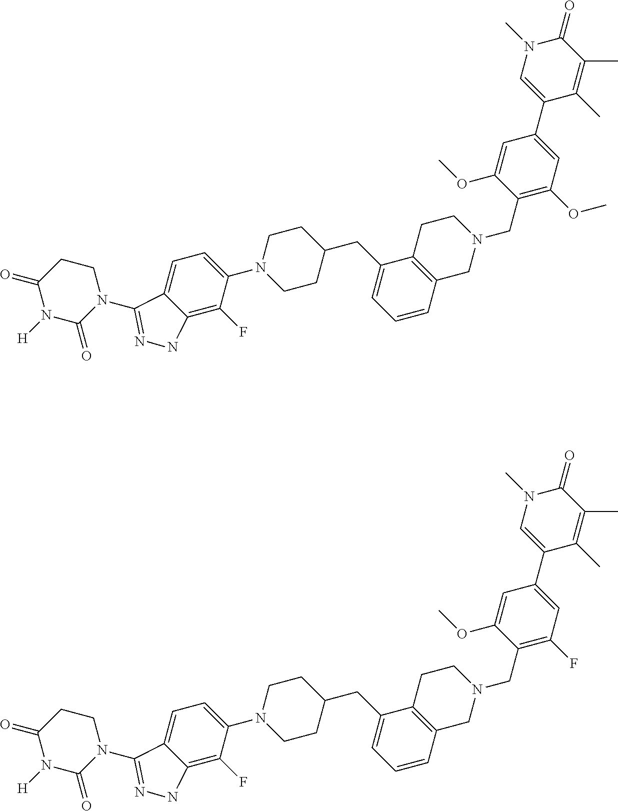 [SMILES: COc1cc(-c2cn(C)c(=O)c(C)c2C)cc(OC)c1CN1CCc2c(CC3CCN(c4ccc5c(N6CCC(=O)NC6=O)n[nH]c5c4F)CC3)cccc2C1 ; COc1cc(-c2cn(C)c(=O)c(C)c2C)cc(F)c1CN1CCc2c(CC3CCN(c4ccc5c(N6CCC(=O)NC6=O)n[nH]c5c4F)CC3)cccc2C1]

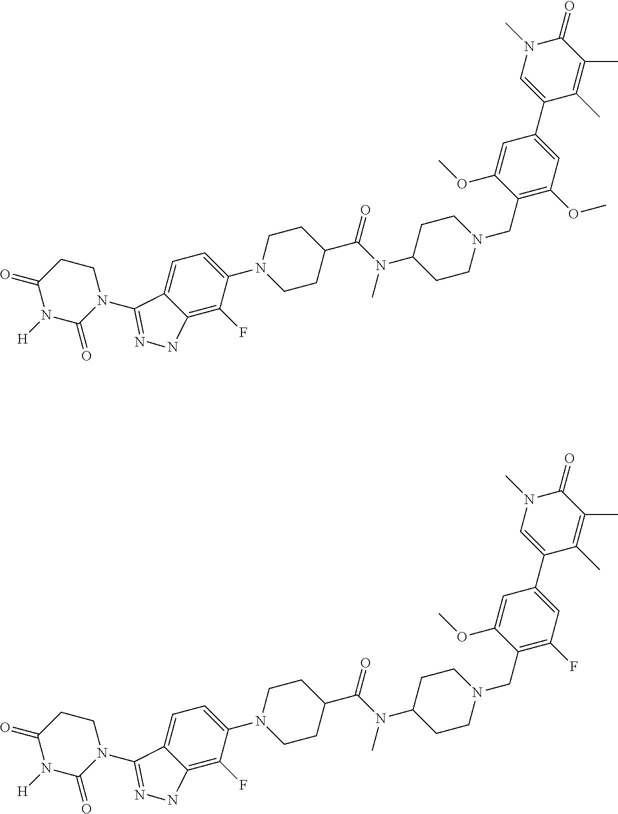 [SMILES: COc1cc(-c2cn(C)c(=O)c(C)c2C)cc(OC)c1CN1CCC(N(C)C(=O)C2CCN(c3ccc4c(N5CCC(=O)NC5=O)n[nH]c4c3F)CC2)CC1 ; COc1cc(-c2cn(C)c(=O)c(C)c2C)cc(F)c1CN1CCC(N(C)C(=O)C2CCN(c3ccc4c(N5CCC(=O)NC5=O)n[nH]c4c3F)CC2)CC1]

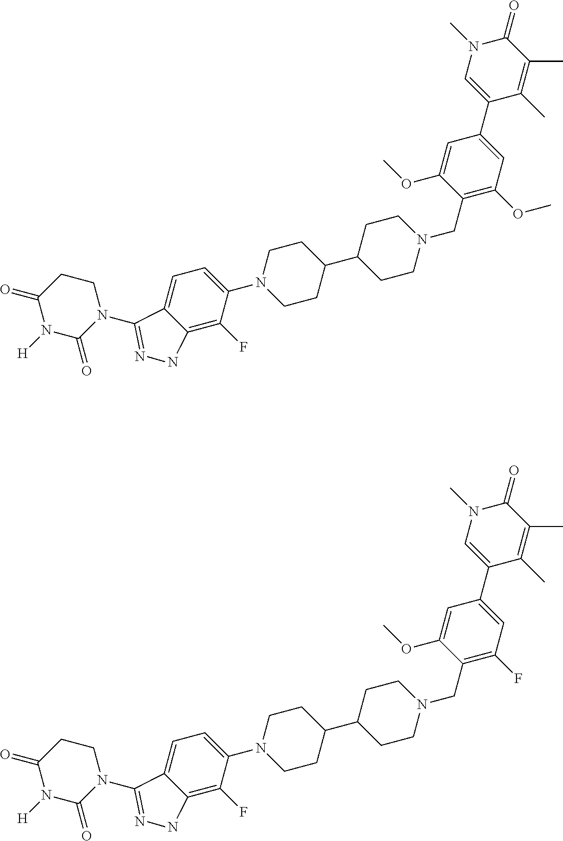 [SMILES: COc1cc(-c2cn(C)c(=O)c(C)c2C)cc(OC)c1CN1CCC(C2CCN(c3ccc4c(N5CCC(=O)NC5=O)n[nH]c4c3F)CC2)CC1 ; COc1cc(-c2cn(C)c(=O)c(C)c2C)cc(F)c1CN1CCC(C2CCN(c3ccc4c(N5CCC(=O)NC5=O)n[nH]c4c3F)CC2)CC1]

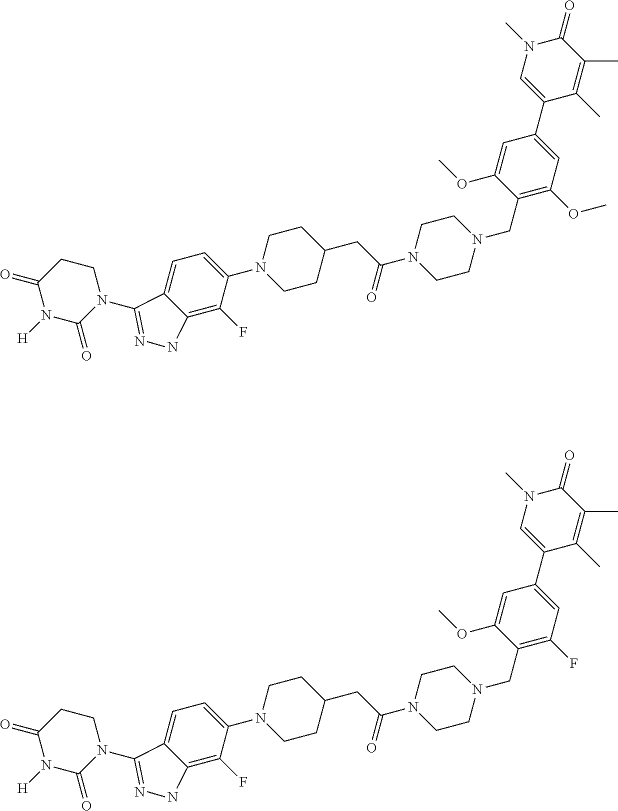 [SMILES: COc1cc(-c2cn(C)c(=O)c(C)c2C)cc(OC)c1CN1CCN(C(=O)CC2CCN(c3ccc4c(N5CCC(=O)NC5=O)n[nH]c4c3F)CC2)CC1 ; COc1cc(-c2cn(C)c(=O)c(C)c2C)cc(F)c1CN1CCN(C(=O)CC2CCN(c3ccc4c(N5CCC(=O)NC5=O)n[nH]c4c3F)CC2)CC1]

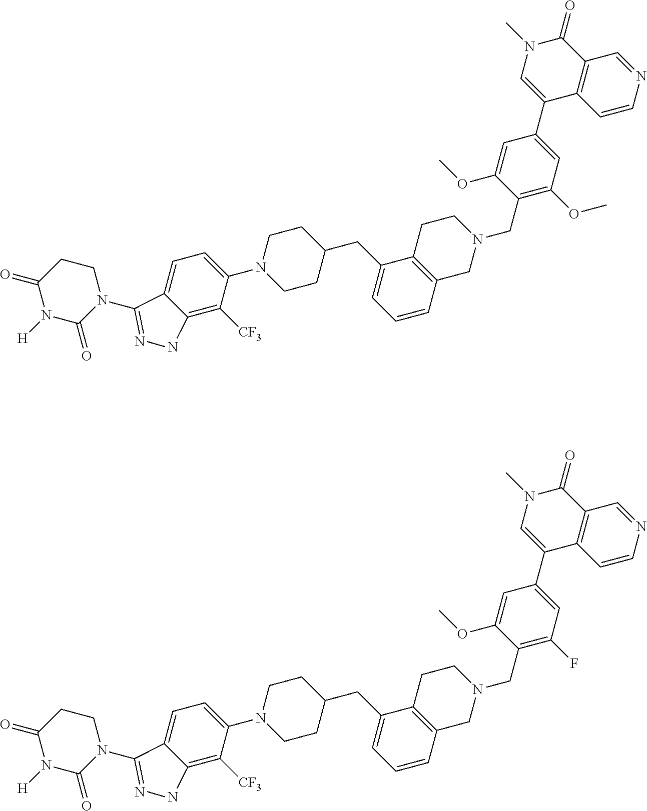 [SMILES: COc1cc(-c2cn(C)c(=O)c3cnccc23)cc(OC)c1CN1CCc2c(CC3CCN(c4ccc5c(N6CCC(=O)NC6=O)n[nH]c5c4C(F)(F)F)CC3)cccc2C1 ; COc1cc(-c2cn(C)c(=O)c3cnccc23)cc(F)c1CN1CCc2c(CC3CCN(c4ccc5c(N6CCC(=O)NC6=O)n[nH]c5c4C(F)(F)F)CC3)cccc2C1]

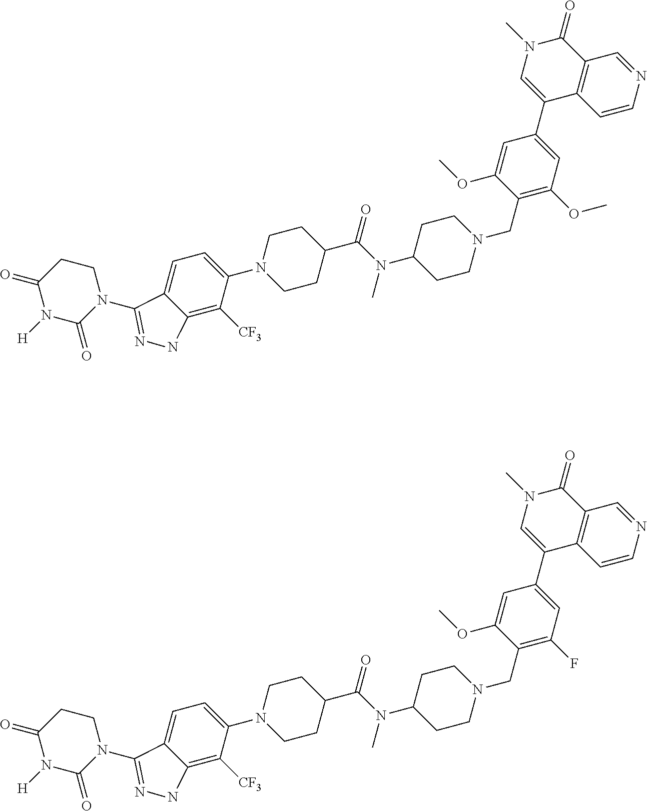 [SMILES: COc1cc(-c2cn(C)c(=O)c3cnccc23)cc(OC)c1CN1CCC(N(C)C(=O)C2CCN(c3ccc4c(N5CCC(=O)NC5=O)n[nH]c4c3C(F)(F)F)CC2)CC1 ; COc1cc(-c2cn(C)c(=O)c3cnccc23)cc(F)c1CN1CCC(N(C)C(=O)C2CCN(c3ccc4c(N5CCC(=O)NC5=O)n[nH]c4c3C(F)(F)F)CC2)CC1]

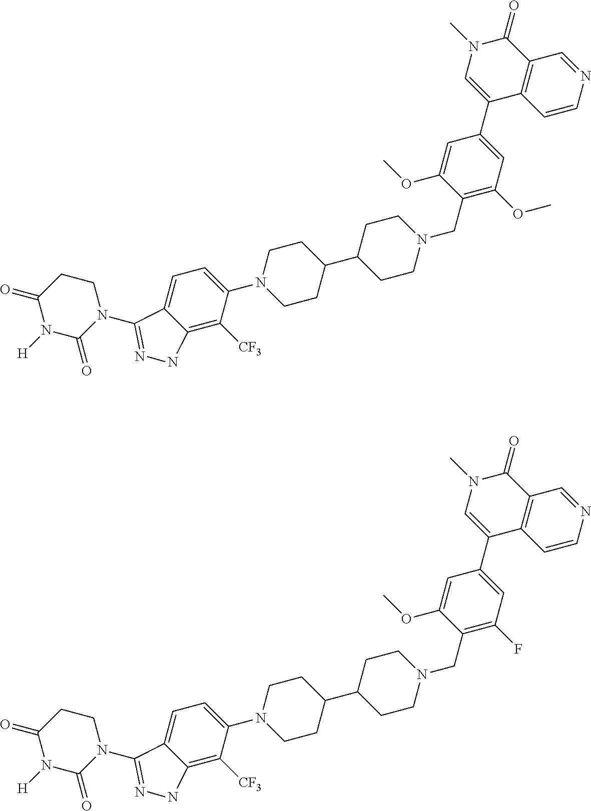 [SMILES: COc1cc(-c2cn(C)c(=O)c3cnccc23)cc(OC)c1CN1CCC(C2CCN(c3ccc4c(N5CCC(=O)NC5=O)n[nH]c4c3C(F)(F)F)CC2)CC1 ; COc1cc(-c2cn(C)c(=O)c3cnccc23)cc(F)c1CN1CCC(C2CCN(c3ccc4c(N5CCC(=O)NC5=O)n[nH]c4c3C(F)(F)F)CC2)CC1]

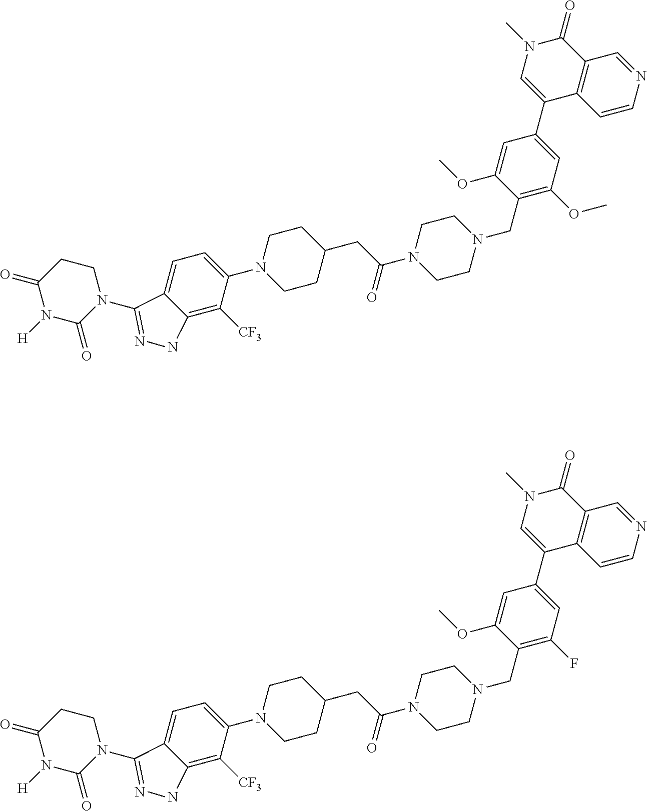 [SMILES: COc1cc(-c2cn(C)c(=O)c3cnccc23)cc(OC)c1CN1CCN(C(=O)CC2CCN(c3ccc4c(N5CCC(=O)NC5=O)n[nH]c4c3C(F)(F)F)CC2)CC1 ; COc1cc(-c2cn(C)c(=O)c3cnccc23)cc(F)c1CN1CCN(C(=O)CC2CCN(c3ccc4c(N5CCC(=O)NC5=O)n[nH]c4c3C(F)(F)F)CC2)CC1]

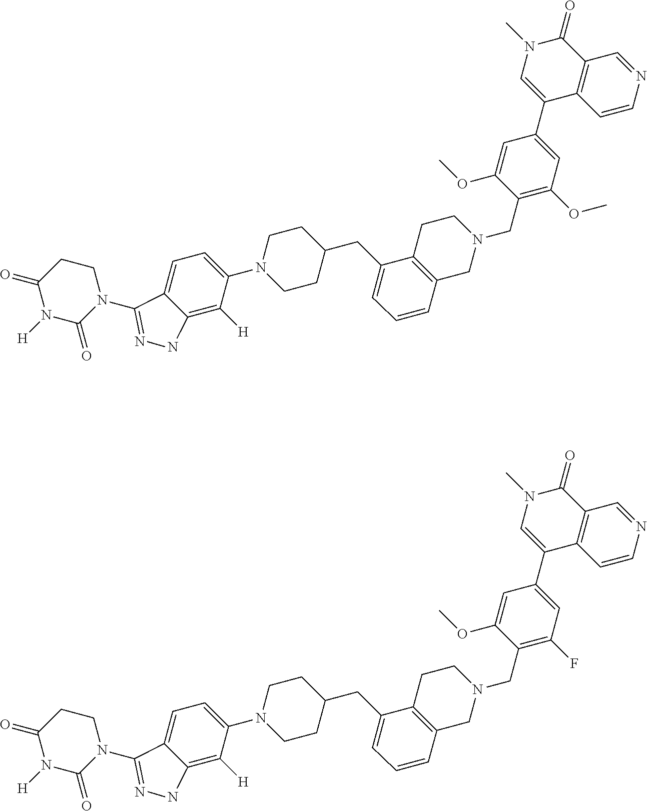 [SMILES: COc1cc(-c2cn(C)c(=O)c3cnccc23)cc(OC)c1CN1CCc2c(CC3CCN(c4ccc5c(N6CCC(=O)NC6=O)n[nH]c5c4)CC3)cccc2C1 ; COc1cc(-c2cn(C)c(=O)c3cnccc23)cc(F)c1CN1CCc2c(CC3CCN(c4ccc5c(N6CCC(=O)NC6=O)n[nH]c5c4)CC3)cccc2C1]

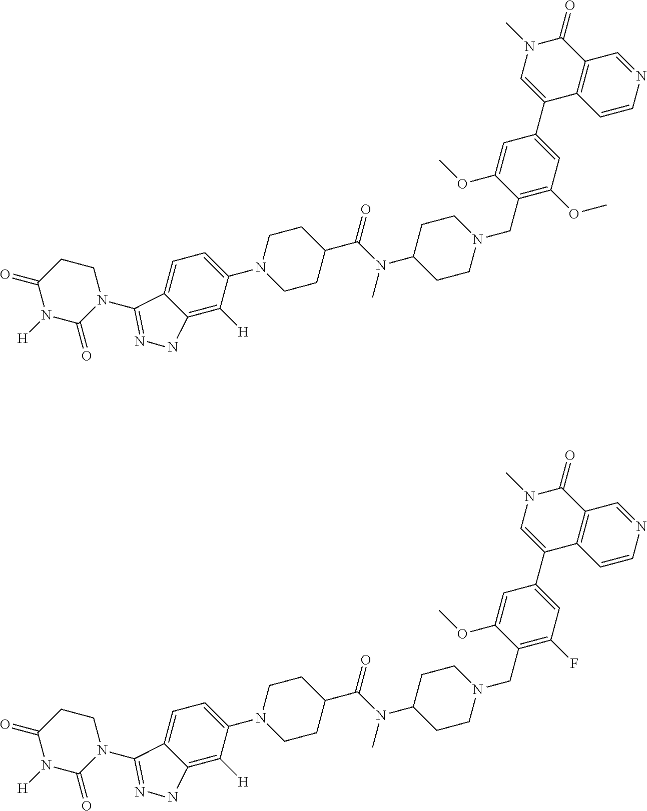 [SMILES: COc1cc(-c2cn(C)c(=O)c3cnccc23)cc(OC)c1CN1CCC(N(C)C(=O)C2CCN(c3ccc4c(N5CCC(=O)NC5=O)n[nH]c4c3)CC2)CC1 ; COc1cc(-c2cn(C)c(=O)c3cnccc23)cc(F)c1CN1CCC(N(C)C(=O)C2CCN(c3ccc4c(N5CCC(=O)NC5=O)n[nH]c4c3)CC2)CC1]

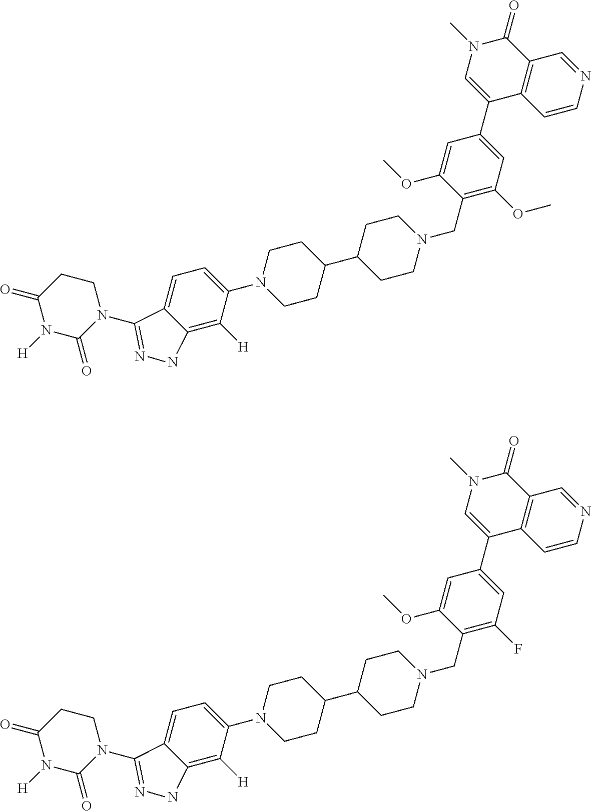 [SMILES: COc1cc(-c2cn(C)c(=O)c3cnccc23)cc(OC)c1CN1CCC(C2CCN(c3ccc4c(N5CCC(=O)NC5=O)n[nH]c4c3)CC2)CC1 ; COc1cc(-c2cn(C)c(=O)c3cnccc23)cc(F)c1CN1CCC(C2CCN(c3ccc4c(N5CCC(=O)NC5=O)n[nH]c4c3)CC2)CC1]

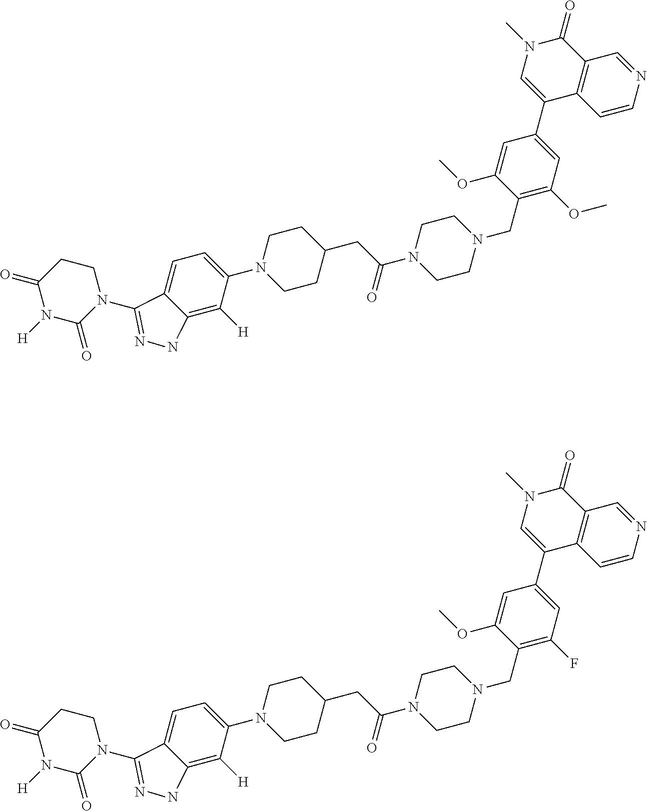 [SMILES: COc1cc(-c2cn(C)c(=O)c3cnccc23)cc(OC)c1CN1CCN(C(=O)CC2CCN(c3ccc4c(N5CCC(=O)NC5=O)n[nH]c4c3)CC2)CC1 ; COc1cc(-c2cn(C)c(=O)c3cnccc23)cc(F)c1CN1CCN(C(=O)CC2CCN(c3ccc4c(N5CCC(=O)NC5=O)n[nH]c4c3)CC2)CC1]

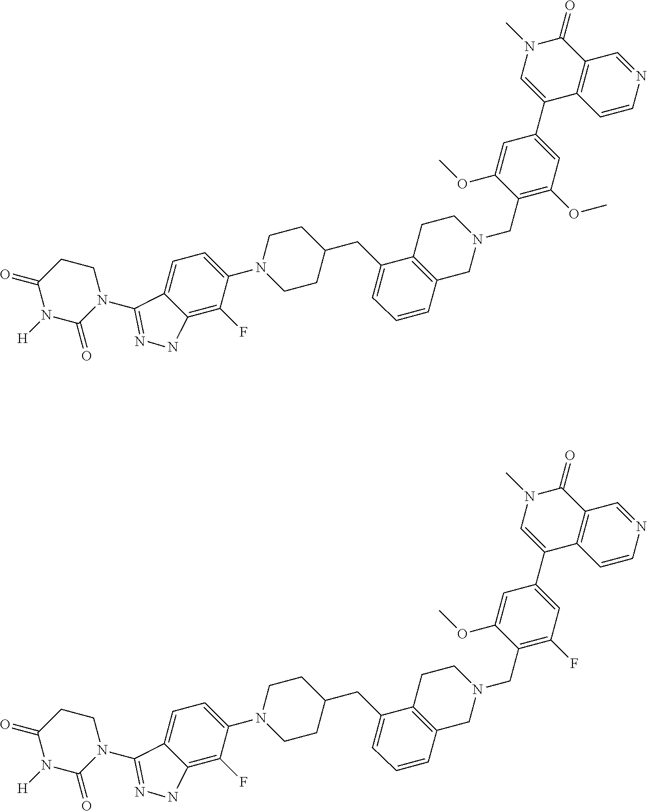 [SMILES: COc1cc(-c2cn(C)c(=O)c3cnccc23)cc(OC)c1CN1CCc2c(CC3CCN(c4ccc5c(N6CCC(=O)NC6=O)n[nH]c5c4F)CC3)cccc2C1 ; COc1cc(-c2cn(C)c(=O)c3cnccc23)cc(F)c1CN1CCc2c(CC3CCN(c4ccc5c(N6CCC(=O)NC6=O)n[nH]c5c4F)CC3)cccc2C1]

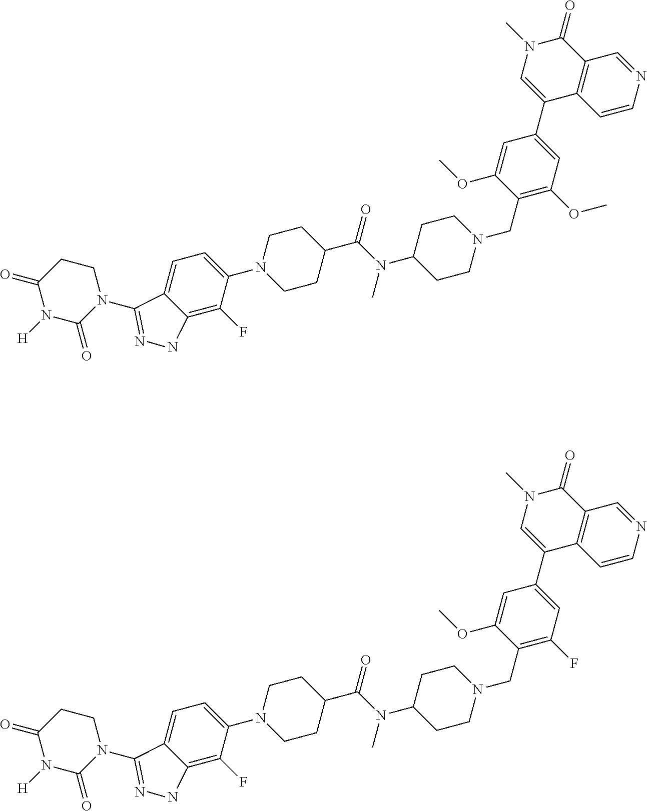 [SMILES: COc1cc(-c2cn(C)c(=O)c3cnccc23)cc(OC)c1CN1CCC(N(C)C(=O)C2CCN(c3ccc4c(N5CCC(=O)NC5=O)n[nH]c4c3F)CC2)CC1 ; COc1cc(-c2cn(C)c(=O)c3cnccc23)cc(F)c1CN1CCC(N(C)C(=O)C2CCN(c3ccc4c(N5CCC(=O)NC5=O)n[nH]c4c3F)CC2)CC1]

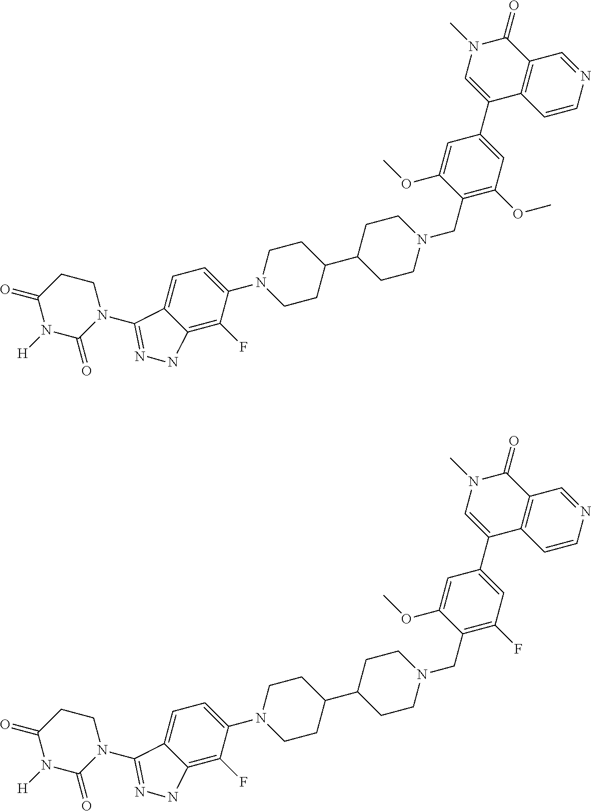 [SMILES: COc1cc(-c2cn(C)c(=O)c3cnccc23)cc(OC)c1CN1CCC(C2CCN(c3ccc4c(N5CCC(=O)NC5=O)n[nH]c4c3F)CC2)CC1 ; COc1cc(-c2cn(C)c(=O)c3cnccc23)cc(F)c1CN1CCC(C2CCN(c3ccc4c(N5CCC(=O)NC5=O)n[nH]c4c3F)CC2)CC1]

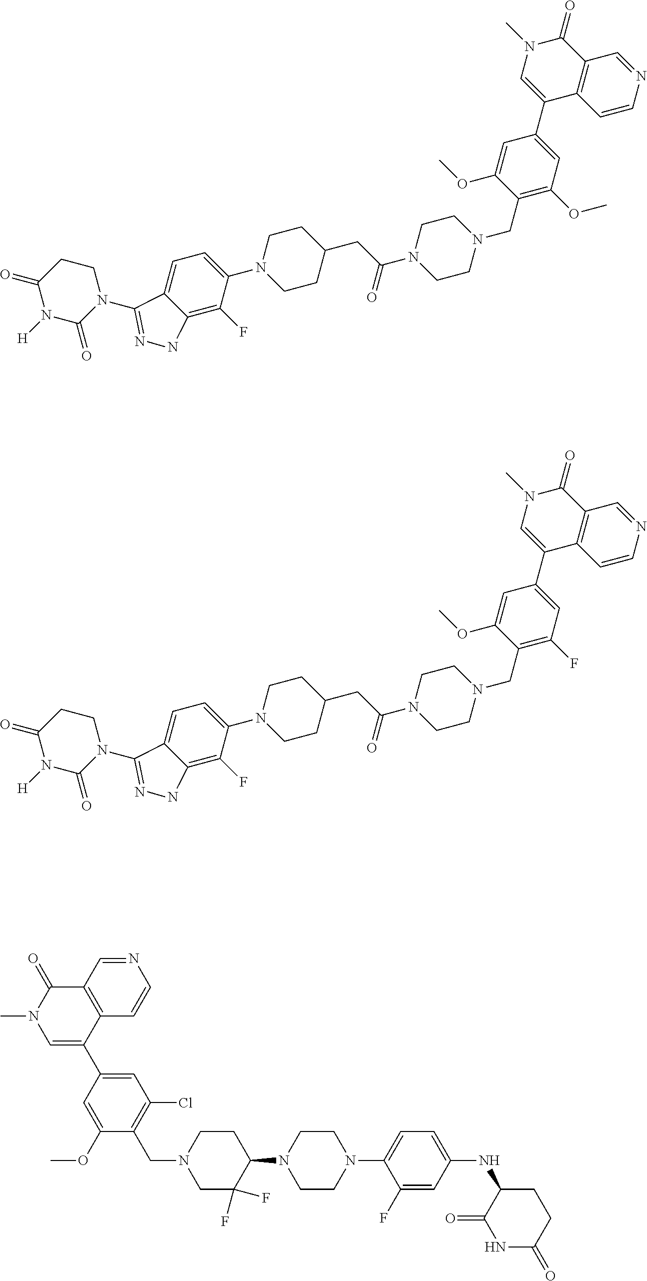 [SMILES: COc1cc(-c2cn(C)c(=O)c3cnccc23)cc(OC)c1CN1CCN(C(=O)CC2CCN(c3ccc4c(N5CCC(=O)NC5=O)n[nH]c4c3F)CC2)CC1 ; COc1cc(-c2cn(C)c(=O)c3cnccc23)cc(F)c1CN1CCN(C(=O)CC2CCN(c3ccc4c(N5CCC(=O)NC5=O)n[nH]c4c3F)CC2)CC1 ; COc1cc(-c2cn(C)c(=O)c3cnccc23)cc(Cl)c1CN1CC[C@@H](N2CCN(c3ccc(N[C@H]4CCC(=O)NC4=O)cc3F)CC2)C(F)(F)C1]

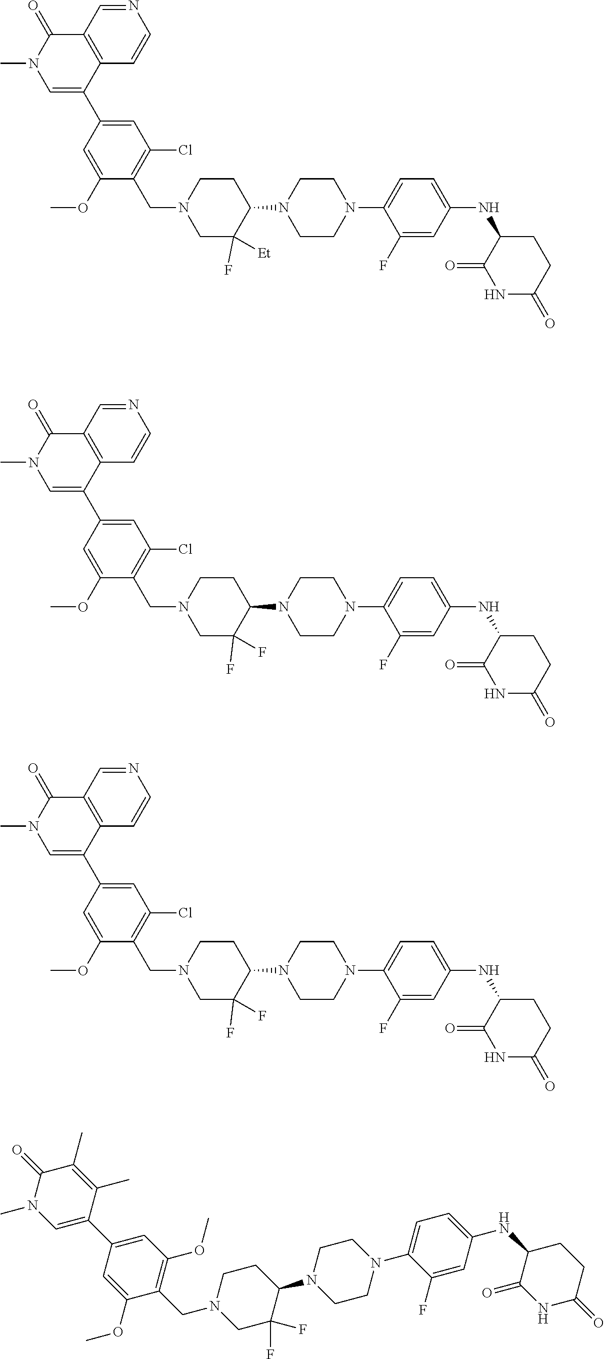 [SMILES: CCC1(F)CN(Cc2c(Cl)cc(-c3cn(C)c(=O)c4cnccc34)cc2OC)CC[C@@H]1N1CCN(c2ccc(N[C@H]3CCC(=O)NC3=O)cc2F)CC1 ; COc1cc(-c2cn(C)c(=O)c3cnccc23)cc(Cl)c1CN1CC[C@@H](N2CCN(c3ccc(N[C@@H]4CCC(=O)NC4=O)cc3F)CC2)C(F)(F)C1 ; COc1cc(-c2cn(C)c(=O)c3cnccc23)cc(Cl)c1CN1CC[C@H](N2CCN(c3ccc(N[C@@H]4CCC(=O)NC4=O)cc3F)CC2)C(F)(F)C1 ; COc1cc(-c2cn(C)c(=O)c(C)c2C)cc(OC)c1CN1CC[C@@H](N2CCN(c3ccc(N[C@H]4CCC(=O)NC4=O)cc3F)CC2)C(F)(F)C1]

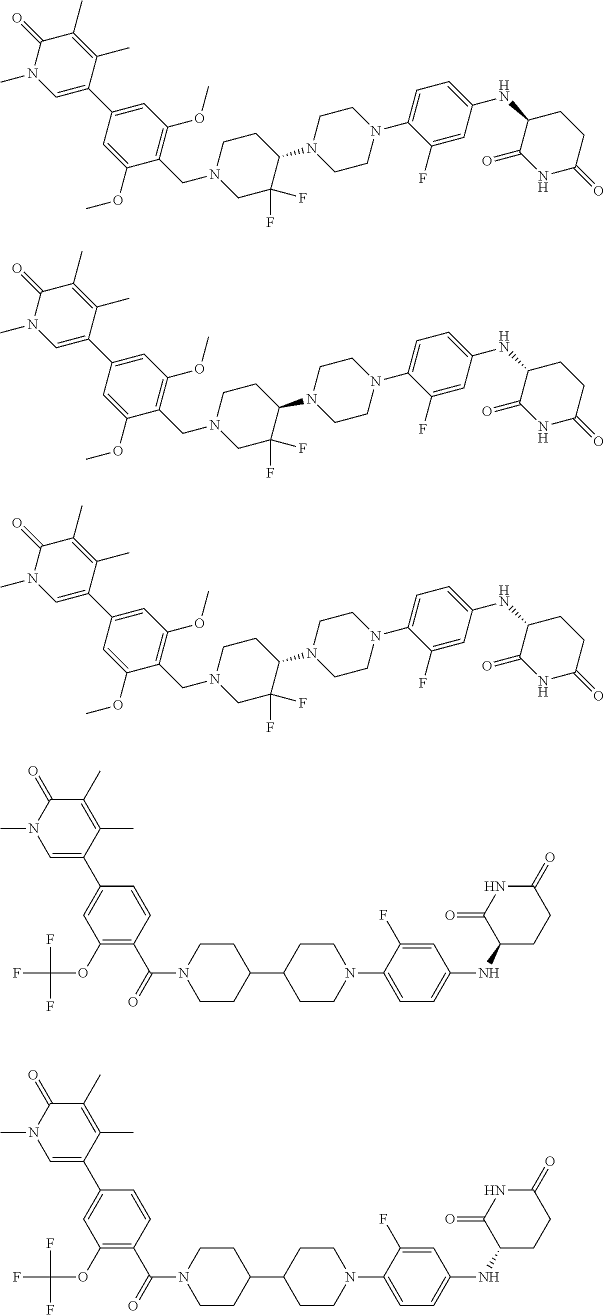

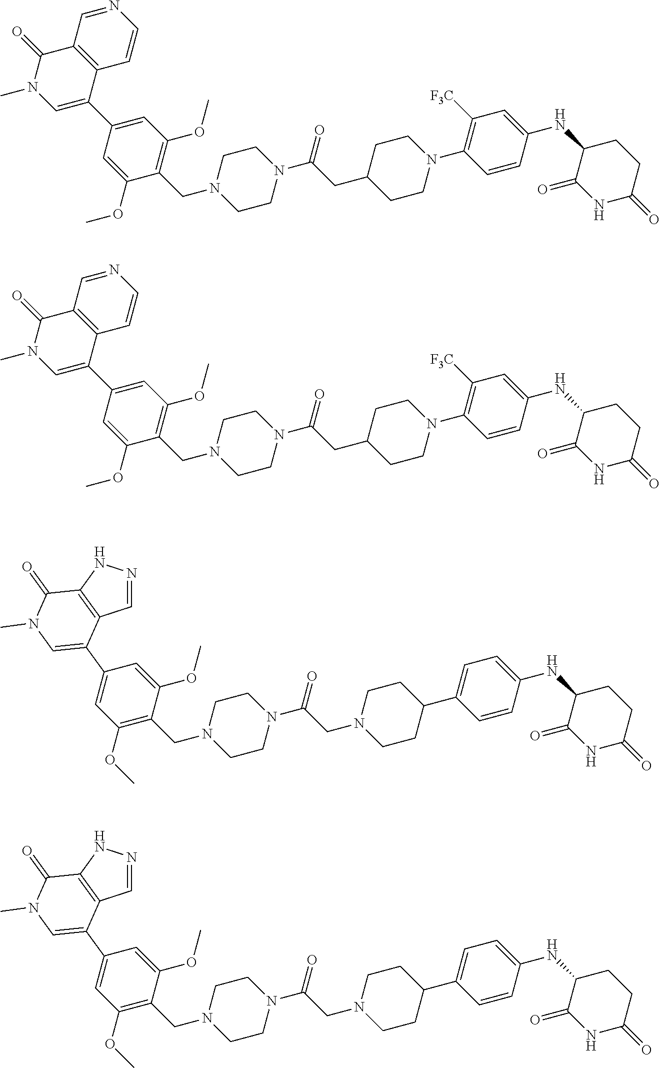 [SMILES: COc1cc(-c2cn(C)c(=O)c3cnccc23)cc(OC)c1CN1CCN(C(=O)CC2CCN(c3ccc(N[C@H]4CCC(=O)NC4=O)cc3C(F)(F)F)CC2)CC1 ; COc1cc(-c2cn(C)c(=O)c3cnccc23)cc(OC)c1CN1CCN(C(=O)CC2CCN(c3ccc(N[C@@H]4CCC(=O)NC4=O)cc3C(F)(F)F)CC2)CC1 ; COc1cc(-c2cn(C)c(=O)c3[nH]ncc23)cc(OC)c1CN1CCN(C(=O)CN2CCC(c3ccc(N[C@H]4CCC(=O)NC4=O)cc3)CC2)CC1 ; COc1cc(-c2cn(C)c(=O)c3[nH]ncc23)cc(OC)c1CN1CCN(C(=O)CN2CCC(c3ccc(N[C@@H]4CCC(=O)NC4=O)cc3)CC2)CC1]

or a pharmaceutically acceptable salt thereof.

Any suitable BRD9 inhibitor, e.g., a BRD9 inhibitor with an ionizable functional group, may be administered as a pharmaceutically acceptable salt, e.g., a salt of an acid selected from the group consisting of formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, ciitric acid, glycolic acid, glucuronic acid, ascorbic acid, benzoic acid, phthalic acid, salicylic acid, anthranilic acid, benzensulfonic acid, p-toluenesulfonic acid, methanesulfonic acid, dichloroacetic acid, aminooxy acetic acid, hydrochloric acid, hydrobromic acid, sulfuric acid, phosphoric acid, nitric acid, carbonic acid, and boric acid.

### Androgen Receptor-Independent Prostate Cancer

The androgen receptor is a transcription factor that effectuates changes in DNA expression upon binding to androgenic ligands such as testosterone and dihydroxytestosterone. Subtypes of prostate cancer may grow and spread independently of gene expression mediated by the androgen receptor. Androgen-independent prostate cancer is prostate cancer that grows and spreads in the absence of a functional androgen receptor. Androgen receptor-independent prostate cancer cannot be successfully treated with androgen starvation therapy or with androgen receptor signaling inhibitors (ARSi).

### Castrate-Resistant Prostate Cancer

Castrate-resistant prostate cancer is a form of prostate cancer that continues to grow even when the amount of testosterone in a subject is reduced to a very low level, e.g., even after surgical or medical castration. The estimated mean survival of a subject diagnosed with castrate-resistant prostate cancer is approximately 9 to 36 months, and the quality of life for subjects diagnosed with castrate-resistant prostate cancer and receiving the standard of care is poor.

### Small Cell Prostate Cancer

Small cell prostate cancer is an aggressive subtype of prostate cancer that is characterized by cancer cells that are smaller in size than other forms of prostate cancer, e.g., cells that are smaller than 4 lymphocytes in diameter. Small cell prostate cancer cells possess unique and strict morphological features, scant cytoplasm, ill-defined borders, finely granular “salt and pepper” chromatic, absent or inconspicuous nucleoli, frequent nuclear molding, and a high mitotic count.

### Neuroendocrine Prostate Cancer and Adenocarcinoma

Neuroendocrine prostate cancer is a subtype of prostate cancer that develops in the neuroendocrine cells of the prostate. Neuroendocrine prostate cancer rarely arises de novo and often develops from the trans-differentiation of adenocarcinomas. Neuroendocrine prostate cancers are typically not responsive to treatments that target androgen receptor signaling.

Treatment of an adenocarcinoma with a compound that reduces the level and/or activity of BRD9, or a pharmaceutically acceptable salt thereof, may reduce the level of adenocarcinoma to neuroendocrine trans-differentiation. The decrease in the level of adenocarcinoma to neuroendocrine trans-differentiation may be measured by any reproducible means of measurement. For example, after treatment, the decrease in the level of adenocarcinoma to neuroendocrine trans-differentiation may be measured by lower expression levels of CHGA, SYP, and/or ENO2 compared to a subject that is not administered the compound that reduces the level and/or activity of BRD9, or a pharmaceutically acceptable salt thereof. The expression levels of CHGA, SYP, and/or ENO2 may be reduced by greater than 5% or greater (e.g., 10%, 20%, 30%, 40%, 50%, 60%, 70%, 80%, 90%, or greater) after treatment relative to expression levels in a subject that has not received treatment.

In some embodiments, the prostate cancer has been determined to have or predicted to have lower expression levels of AR, KLK2, KLK3, CDH1, CYLD, NKX3-1, SLC45A3, TARP, PTEN, SPDEF, TP53, or RB1, or combinations thereof compared to a subject that does not have prostate cancer.

In some embodiments, the prostate cancer has been determined to have or predicted to have lower expression levels of AR, KLK2, KLK3, CDH1, CYLD, NKX3-1, SLC45A3, TARP, PTEN, SPDEF, TP53, or RB1, or combinations thereof compared to standard levels for prostate cancer.

In some embodiments, the prostate cancer has been determined to have or predicted to have higher expression levels of CHGB, CHGA, SYP, ENO2, PEG10, SNAP25, SRRM4, VGF, VIM, SCGN, PAPPA2, or WNT11, or combinations thereof compared to a subject that does not have prostate cancer.

In some embodiments, the prostate cancer has been determined to have or predicted to have higher expression levels of CHGB, CHGA, SYP, ENO2, PEG10, SNAP25, SRRM4, VGF, VIM, SCGN, PAPPA2, or WNT11, or combinations thereof compared to standard levels for prostate cancer.

In some embodiments, the prostate cancer has been determined to have or predicted to have higher expression levels of ASCL1, EZH2, DLX5, DLX6, SOX2, SOX11, NKX2-2, HES6, SOX9, KDM3A, FOXA2, AURKA, MYCN, MYC, AKT, POU3F2/BRN2, NANOG, ONECUT2, or NKX2-1, or combinations thereof compared to a subject that does not have prostate cancer.

In some embodiments, the prostate cancer has been determined to have or predicted to have higher expression levels of ASCL1, EZH2, DLX5, DLX6, SOX2, SOX11, NKX2-2, HES6, SOX9, KDM3A, FOXA2, AURKA, MYCN, MYC, AKT, POU3F2/BRN2, NANOG, ONECUT2, or NKX2-1, or combinations thereof compared to standard levels for prostate cancer.

In some embodiments, the prostate cancer has been determined to have or predicted to have lower expression levels of RE1 silencing transcription factor (REST).

In some embodiments, the prostate cancer has been determined to have or predicted to have expression of AR and KLK3 (PSA).

In some embodiments, the prostate cancer has been determined to have or predicted to have undetectable expression of CHGA and SYP.

In some embodiments, the prostate cancer has been determined to have or predicted to have expression of AR, KLK3 (PSA), CHGA and SYP.

In some embodiments, the prostate cancer has been determined to have or predicted to have undetectable expression of AR and KLK3 (PSA).

In some embodiments, the prostate cancer has been determined to have or predicted to have expression of CHGA and SYP.

In some embodiments, the prostate cancer has been determined to have or predicted to have undetectable expression of AR, KLK3 (PSA), CHGA and SYP.

In some embodiments, the prostate cancer has been determined or predicted to have a high level of TMPRSS2-ERG fusions.

In some embodiments, expression of BRD9, GLTSCR1, CXXC5 or TET2 is increased in the prostate cancer compared to a subject that does not have prostate cancer.

In some embodiments, expression of BRD9 is increased in the prostate cancer compared to a subject that does not have prostate cancer.

In some embodiments, expression of GLTSCR1 is increased in the prostate cancer compared to a subject that does not have prostate cancer.

In some embodiments, expression of TET2, CXXC5, H3K27ac, ID1, PFN2, or ID3 in the subject is increased in the prostate cancer determined to or predicted to be resistant to enzalutamide compared to a prostate cancer that responds to treatment with enzalutamide.

In some embodiments, the prostate cancer has been determined to have or predicted to have undetectable expression of PTEN.

### ERG Inhibitors

The ETS-related gene (ERG) encodes a transcription factor with oncogenic properties that plays a role in the development of prostate cancer. In prostate cancers, the ETS gene commonly fuses with the promoter region of the TMPRRSS2 gene, which leads to overexpression of the ETS transcription factor. In some embodiments, the present invention provides methods for treating androgen receptor-independent prostate cancer that include further administering an inhibitor or degrader of ERG to a subject with androgen receptor-independent prostate cancer. A nonlimiting example of an ERG inhibitor that may be administered to a subject in accordance with the methods of the present disclosure is ERGi-USU (1-[2-Thiazolylazo]-2-naphthol).

### Androgen Receptor (AR) Degraders

In some embodiments, the present invention provides methods for treating androgen receptor-independent prostate cancer that include further administering a degrader of the androgen receptor to the subject. Nonlimiting examples of AR degraders that may be administered to a subject in accordance with the methods of the present disclosure include bavdegalutamide (ARV-110), ARV-766, and AR-V7.

### JAK-STAT Pathway Inhibitors

Aberrant activation of the Janus kinase/signal transducer and activator of transcription (JAK-STAT) pathway is related to tumor growth and disease progression in prostate cancer. Accordingly, in some embodiments, the methods of the present invention include further administering to the subject an inhibitor of the JAK-STAT pathway. Nonlimiting examples of JAK-STAT pathway inhibitors that may be administered to a subject in accordance with the methods of the present invention include AG490, AZD1480, AZD4205, baricitinib, dasatinib, fedratinib, filgotinib, itacitnib, lestaurtinib, momelotinib, pacritinib, peficitinib, ruxolitinib, siltuximab, tofacitinib, upadacitinib, or WP1066.

### MAPK Pathway Inhibitors

The mitogen activated protein kinase (MAPK) pathway includes the proteins p38, JNK, and ERK, and is implicated in the progression of prostate cancer. Accordingly, in some embodiments, the methods of the present invention include further administering to the subject an inhibitor of the MAPK pathway. Nonlimiting examples of MAPK pathway inhibitors that may be administered to a subject in accordance with the methods of the present invention include a Farnesyltransferase inhibitor (FTI), Sorafenib, Vemurafenib, PLX8394, Dabrafenib, Ulixertinib, Simvastatin, Alisertib, or Teriflunomide.

### Methods of Treatment

The present disclosure features methods of treating disorders related to BRD9 such as cancer, e.g., androgen receptor-independent prostate cancer and/or neuroendocrine prostate cancer, in a subject in need thereof.

Cancer is a group of diseases characterized by the harmful, abnormal, uncontrolled, and undesirable growth of cells. In some embodiments, the uncontrolled growth is due to cells that divide and proliferate in the absence of signals (e.g., growth factors) instructing them to do so. In some embodiments, the uncontrolled growth is due to cells which fail to respond to signals instructing them to stop growing and/or engage in programmed cell death (i.e., apoptosis). In some embodiments, cancer cells may spread throughout the body (i.e., metastasize). In some embodiments, cancer cells may form tumors. In some embodiments, cancer cells may form solid tumors.

In some embodiments, the compound is administered in an amount and for a time effective to result in one of (or more, e.g., two or more, three or more, four or more of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of a subject, (j) slowed progression of cancer, (k) reduced recurrence of cancer, (l) decreased rate of metastatic tumor seeding, (m) decreased metastatic tumor nodule formation, (n) decreased spread of metastatic tumor nodule formation, and (o) decreased metastatic colonization.

Treating cancer, e.g., treating androgen receptor-independent prostate cancer with a compound that reduces the level and/or activity of BRD9, or a pharmaceutically acceptable salt thereof, can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer, e.g., treating androgen receptor-independent prostate cancer with a compound that reduces the level and/or activity of BRD9, or a pharmaceutically acceptable salt thereof, may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer, e.g., treating androgen receptor-independent prostate cancer with a compound that reduces the level and/or activity of BRD9, or a pharmaceutically acceptable salt thereof, can result in a decrease in the spread of metastatic nodules, e.g., a decrease in the number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer, e.g., treating androgen receptor-independent prostate cancer with a compound that reduces the level and/or activity of BRD9, or a pharmaceutically acceptable salt thereof, can result in a decrease in the level of neuroendocrine prostate cancer cells in the subject. For example, after treatment, the level of neuroendocrine prostate cancer cells in the subject is reduced by 5% or greater (e.g., 10%, 20%, 30%, 40%, 50%, 60%, 70%, 80%, 90% or greater) relative to level prior to treatment. The level of neuroendocrine prostate cancer cells may be measured by any reproducible means of measurement. For example, the level of neuroendocrine prostate cancer cells may be measured by obtaining a sample of prostate tissue from a subject with prostate cancer and conducting cell sorting experiments to determine the proportion of the cells in the sample that are neuroendocrine prostate cancer cells.

Treating cancer, e.g., treating androgen receptor-independent prostate cancer with a compound that reduces the level and/or activity of BRD9, or a pharmaceutically acceptable salt thereof, can result in the treatment of a cancer that had previously failed to respond to an anti-cancer therapy, e.g., an anti-cancer therapy that did not target BRD9. For example, the methods of the present invention may result in the treatment of a cancer that had previously failed to respond to active surveillance, surgery, radiation therapy, high-intensity focused ultrasound (HIFU), cryotherapy, hormone therapy, chemotherapy, immunotherapy, vaccine treatment, immune checkpoint inhibitors, targeted therapy drugs, or bone-directed treatment.

Treating cancer, e.g., treating androgen receptor-independent prostate cancer with a compound that reduces the level and/or activity of BRD9, can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound described herein. An increase in average survival time of a population may also be measured, for example, by calculating for a population the average length of survival following completion of a first round of treatment with a compound that reduces the level and/or activity of BRD9, or a pharmaceutically acceptable salt thereof.

Treating cancer, e.g., treating androgen receptor-independent prostate cancer with a compound that reduces the level and/or activity of BRD9, can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of a compound described herein. A decrease in the mortality rate of a population may also be measured, for example, by calculating for a population the average number of disease-related deaths per unit time following completion of a first round of treatment with a compound that reduces the level and/or activity of BRD9, or a pharmaceutically acceptable salt thereof.

Treating cancer, e.g., treating androgen receptor-independent prostate cancer with a compound that reduces the level and/or activity of BRD9, or a pharmaceutical salt thereof, may result in a reduced recurrence of cancer relative to the recurrence of cancer in a subject that has not been treated with a compound that reduces the level and/or activity of BRD9, or a pharmaceutical salt thereof. A decrease in the recurrence of cancer may be measured, for example, by monitoring the number of incidences of recurrence of cancer over a period of time (e.g., one week, one month, one year, five years) in a population of cancer subjects with cancer treated with a compound that reduces the level and/or activity of BRD9, or a pharmaceutically acceptable salt thereof, and comparing that number of incidences to the number of incidences of recurrence of cancer in a population of subjects with cancer who are not administered a compound that reduces the level and/or activity of BRD9, or a pharmaceutically acceptable salt thereof, over the same period of time.

### Combination Therapies

A method of the invention can be used alone or in combination with an additional therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65: S3-S6 (2005)). In this case, dosages of the compounds when combined should provide a therapeutic effect.

In some embodiments, the androgen-independent prostate cancer and/or neuroendocrine prostate cancer has failed to respond to a previous treatment. In some embodiments, the previous treatment is a second therapeutic agent. In some embodiments the previous treatment is an anti-cancer therapy. In some embodiments, a subject is further administered an anti-cancer therapy. In some embodiments, the anti-cancer therapy is administered prior to the administering of a compound of the present disclosure. In some embodiments, the anti-cancer therapy is administered in addition to the administering of a compound of the present disclosure. In some embodiments, the anti-cancer therapy is administered subsequent to the administering of a compound of the present disclosure. In some embodiments, the anti-cancer therapy is active surveillance, surgery, radiation therapy, high-intensity focused ultrasound (HIFU), cryotherapy, hormone therapy, chemotherapy, immunotherapy, vaccine treatment, immune checkpoint inhibitors, targeted therapy drugs, or bone-directed treatment. In some embodiments, an anti-cancer therapy is abiraterone acetate, alendronate, apalutamide, bicalutamide, cabazitaxel, carboplatin, cisplatin, darolutamide, degarelix, denosumab, docetaxel, enzalutamide, etoposide, flutamide, goserelin acetate, ibandronate, leuprolide acetate, lynparza, mitoxantrone hydrochloride, nilutamide, olaparib, pamidronate, radium 223 dichloride, relugolix, risedronate, rucaparib camsylate, sipuleucel-T, or zoledronic acid, or combinations thereof.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; or zoledronic acid and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18: 233a (1999), and Douillard et al., Lancet 355 (9209): 1041-1047 (2000).

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-I-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOLIRIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The second agent may be a therapeutic agent which is a non-drug treatment. For example, the second therapeutic agent is radiation therapy, cryotherapy, hyperthermia, and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or fusion a protein such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In some embodiments, the anti-cancer therapy is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The pharmaceutical compositions described herein are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the invention, the described compounds or salts, solvates, or prodrugs thereof may be administered to a subject in a variety of forms depending on the selected route of administration, as will be understood by those skilled in the art. The compounds described herein may be administered, for example, by oral, parenteral, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Solutions of a compound described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response. In general, satisfactory results may be obtained when the compounds described herein are administered to a human at a daily dosage of, for example, between 0.05 mg and 3000 mg (measured as the solid form). Dose ranges include, for example, between 10-1000 mg.

Alternatively, the dosage amount can be calculated using the body weight of the subject. For example, the dose of a compound, or pharmaceutical composition thereof, administered to a subject may range from 0.1-100 mg/kg.

In some embodiments, the effective amount of a compound of the present disclosure or a pharmaceutically acceptable salt thereof is administered in a dose of 20-120 mg/kg (e.g., 20-60 mg/kg, 20-40 mg/kg, 40-80 mg/kg, 40-60 mg/kg, 60-80 mg/kg, or 80-120 mg/kg).

In some embodiments, the effective amount of a compound of the present disclosure or a pharmaceutically acceptable salt thereof is administered in a dose of 20 mg/kg. In some embodiments, the effective amount of a compound of the present disclosure or a pharmaceutically acceptable salt thereof is administered in a dose of 40 mg/kg. In some embodiments, the effective amount of a compound of the present disclosure or a pharmaceutically acceptable salt thereof is administered in a dose of 50 mg/kg. In some embodiments, the effective amount of a compound of the present disclosure or a pharmaceutically acceptable salt thereof is administered in a dose of 60 mg/kg. In some embodiments, the effective amount of a compound of the present disclosure or a pharmaceutically acceptable salt thereof is administered in a dose of 80 mg/kg. In some embodiments, the effective amount of a compound of the present disclosure or a pharmaceutically acceptable salt thereof is administered in a dose of 120 mg/kg.

In some embodiments, the effective amount of a compound of the present disclosure or a pharmaceutically acceptable salt thereof is administered at least once per week.

In some embodiments, the effective amount of a compound of the present disclosure or a pharmaceutically acceptable salt thereof is administered at least twice per week.

In some embodiments, the effective amount of a compound of the present disclosure or a pharmaceutically acceptable salt thereof is administered in a dose of 20 mg/kg once per week. In some embodiments, the effective amount of a compound of the present disclosure or a pharmaceutically acceptable salt thereof is administered in a dose of 40 mg/kg once per week. In some embodiments, the effective amount of a compound of the present disclosure or a pharmaceutically acceptable salt thereof is administered in a dose of 50 mg/kg once per week. In some embodiments, the effective amount of a compound of the present disclosure or a pharmaceutically acceptable salt thereof is administered in a dose of 60 mg/kg once per week. In some embodiments, the effective amount of a compound of the present disclosure or a pharmaceutically acceptable salt thereof is administered in a dose of 80 mg/kg once per week. In some embodiments, the effective amount of a compound of the present disclosure or a pharmaceutically acceptable salt thereof is administered in a dose of 120 mg/kg once per week.

In some embodiments, the effective amount of a compound of the present disclosure or a pharmaceutically acceptable salt thereof is administered in a dose of 20 mg/kg twice per week. In some embodiments, the effective amount of a compound of the present disclosure or a pharmaceutically acceptable salt thereof is administered in a dose of 40 mg/kg twice per week. In some embodiments, the effective amount of a compound of the present disclosure or a pharmaceutically acceptable salt thereof is administered in a dose of 50 mg/kg twice per week. In some embodiments, the effective amount of a compound of the present disclosure or a pharmaceutically acceptable salt thereof is administered in a dose of 60 mg/kg twice per week. In some embodiments, the effective amount of a compound of the present disclosure or a pharmaceutically acceptable salt thereof is administered in a dose of 80 mg/kg twice per week. In some embodiments, the effective amount of a compound of the present disclosure or a pharmaceutically acceptable salt thereof is administered in a dose of 120 mg/kg twice per week.

In some embodiments, the effective amount of a compound of the present disclosure of a pharmaceutically acceptable salt thereof is administered to the subject in a 14-day dosing cycle.

In some embodiments, the effective amount of a compound of the present disclosure of a pharmaceutically acceptable salt thereof is administered to the subject in a 2 week dosing cycle.

In some embodiments, the effective amount of a compound of the present disclosure of a pharmaceutically acceptable salt thereof is administered to the subject in a 21-day dosing cycle.

In some embodiments, the effective amount of a compound of the present disclosure of a pharmaceutically acceptable salt thereof is administered to the subject in a 3 week dosing cycle.

In some embodiments, the effective amount of a compound of the present disclosure of a pharmaceutically acceptable salt thereof is administered to the subject in a 28-day dosing cycle.

In some embodiments, the effective amount of a compound of the present disclosure of a pharmaceutically acceptable salt thereof is administered to the subject in a 4 week dosing cycle.

In some embodiments, the effective amount of a compound of the present disclosure of a pharmaceutically acceptable salt thereof is administered to the subject in a one month dosing cycle.

In some embodiments, a formulation of the present disclosure is administered in an amount sufficient to maintain a concentration of at least 1 ng/ml of compound for at least 14 days after administration. In some embodiments, a formulation of the present disclosure is administered in an amount sufficient to maintain a concentration of at least 1 ng/ml of compound for at least 2 weeks after administration. In some embodiments, a formulation of the present disclosure is administered in an amount sufficient to maintain a concentration of at least 1 ng/ml of compound for at least 21 days after administration. In some embodiments, a formulation of the present disclosure is administered in an amount sufficient to maintain a concentration of at least 1 ng/ml of compound for at least 3 weeks after administration. In some embodiments, a formulation of the present disclosure is administered in an amount sufficient to maintain a concentration of at least 1 ng/ml of compound for at least 28 days after administration. In some embodiments, a formulation of the present disclosure is administered in an amount sufficient to maintain a concentration of at least 1 ng/ml of compound for at least 4 weeks after administration. In some embodiments, a formulation of the present disclosure is administered in an amount sufficient to maintain a concentration of at least 1 ng/ml of compound for at least 1 month after administration.

In some embodiments, a formulation of the present disclosure is administered in an amount sufficient to maintain a concentration of at least 2 ng/ml of compound for at least 14 days after administration. In some embodiments, a formulation of the present disclosure is administered in an amount sufficient to maintain a concentration of at least 2 ng/ml of compound for at least 2 weeks after administration. In some embodiments, a formulation of the present disclosure is administered in an amount sufficient to maintain a concentration of at least 2 ng/ml of compound for at least 21 days after administration. In some embodiments, a formulation of the present disclosure is administered in an amount sufficient to maintain a concentration of at least 2 ng/ml of compound for at least 3 weeks after administration. In some embodiments, a formulation of the present disclosure is administered in an amount sufficient to maintain a concentration of at least 2 ng/ml of compound for at least 28 days after administration. In some embodiments, a formulation of the present disclosure is administered in an amount sufficient to maintain a concentration of at least 2 ng/ml of compound for at least 4 weeks after administration. In some embodiments, a formulation of the present disclosure is administered in an amount sufficient to maintain a concentration of at least 2 ng/mL of compound for at least 1 month after administration.

In some embodiments, a formulation of the present disclosure is administered in an amount sufficient to maintain a concentration of at least 3 ng/ml of compound for at least 14 days after administration. In some embodiments, a formulation of the present disclosure is administered in an amount sufficient to maintain a concentration of at least 3 ng/ml of compound for at least 2 weeks after administration. In some embodiments, a formulation of the present disclosure is administered in an amount sufficient to maintain a concentration of at least 3 ng/ml of compound for at least 21 days after administration. In some embodiments, a formulation of the present disclosure is administered in an amount sufficient to maintain a concentration of at least 3 ng/ml of compound for at least 3 weeks after administration. In some embodiments, a formulation of the present disclosure is administered in an amount sufficient to maintain a concentration of at least 3 ng/mL of compound for at least 28 days after administration. In some embodiments, a formulation of the present disclosure is administered in an amount sufficient to maintain a concentration of at least 3 ng/ml of compound for at least 4 weeks after administration. In some embodiments, a formulation of the present disclosure is administered in an amount sufficient to maintain a concentration of at least 3 ng/ml of compound for at least 1 month after administration.

In some embodiments, a formulation of the present disclosure is administered in an amount sufficient to maintain a concentration of at least 5 ng/ml of compound for at least 14 days after administration. In some embodiments, a formulation of the present disclosure is administered in an amount sufficient to maintain a concentration of at least 5 ng/ml of compound for at least 2 weeks after administration. In some embodiments, a formulation of the present disclosure is administered in an amount sufficient to maintain a concentration of at least 5 ng/ml of compound for at least 21 days after administration. In some embodiments, a formulation of the present disclosure is administered in an amount sufficient to maintain a concentration of at least 5 ng/ml of compound for at least 3 weeks after administration. In some embodiments, a formulation of the present disclosure is administered in an amount sufficient to maintain a concentration of at least 5 ng/ml of compound for at least 28 days after administration. In some embodiments, a formulation of the present disclosure is administered in an amount sufficient to maintain a concentration of at least 5 ng/ml of compound for at least 4 weeks after administration. In some embodiments, a formulation of the present disclosure is administered in an amount sufficient to maintain a concentration of at least 5 ng/ml of compound for at least 1 month after administration.

In some embodiments, a formulation of the present disclosure is administered in an amount sufficient to maintain a concentration of at least 10 ng/ml of compound for at least 14 days after administration. In some embodiments, a formulation of the present disclosure is administered in an amount sufficient to maintain a concentration of at least 10 ng/ml of compound for at least 2 weeks after administration. In some embodiments, a formulation of the present disclosure is administered in an amount sufficient to maintain a concentration of at least 10 ng/ml of compound for at least 21 days after administration. In some embodiments, a formulation of the present disclosure is administered in an amount sufficient to maintain a concentration of at least 10 ng/ml of compound for at least 3 weeks after administration. In some embodiments, a formulation of the present disclosure is administered in an amount sufficient to maintain a concentration of at least 10 ng/ml of compound for at least 28 days after administration. In some embodiments, a formulation of the present disclosure is administered in an amount sufficient to maintain a concentration of at least 10 ng/ml of compound for at least 4 weeks after administration. In some embodiments, a formulation of the present disclosure is administered in an amount sufficient to maintain a concentration of at least 10 ng/mL of compound for at least 1 month after administration.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to decrease a BRD9 immunohistochemistry score of the subject. In some embodiments of any of the aspects disclosed herein, the effective amount is an amount sufficient to maintain a plasma concentration of 3 ng/ml of the compound in a subject, over at least 14 days. In some embodiments of any of the aspects disclosed herein, the effective amount is an amount sufficient to maintain a plasma concentration of 3 ng/ml of the compound in a subject, over at least 21 days. In some embodiments of any of the aspects disclosed herein, the effective amount is an amount sufficient to maintain a plasma concentration of 3 ng/ml of the compound in a subject, over at least 28 days.

In one aspect, the present disclosure provides a method of decreasing a BRD9 immunohistochemistry score in a subject, the method comprising administering an effective amount of a compound that reduces the level and/or activity of BRD9, or a pharmaceutically acceptable salt thereof. In some embodiments of any of the aspects disclosed herein, the effective amount is an amount sufficient to maintain a plasma concentration of 3 ng/ml of the compound in a subject, over at least 14 days. In some embodiments of any of the aspects disclosed herein, the effective amount is an amount sufficient to maintain a plasma concentration of 3 ng/ml of the compound in a subject, over at least 21 days. In some embodiments of any of the aspects disclosed herein, the effective amount is an amount sufficient to maintain a plasma concentration of 3 ng/ml of the compound in a subject, over at least 28 days.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to reduce the level of BRD9 expression in the subject. In some embodiments of any of the aspects disclosed herein, the effective amount is an amount sufficient to maintain a plasma concentration of 3 ng/ml of the compound in a subject, over at least 14 days. In some embodiments of any of the aspects disclosed herein, the effective amount is an amount sufficient to maintain a plasma concentration of 3 ng/ml of the compound in a subject, over at least 21 days. In some embodiments of any of the aspects disclosed herein, the effective amount is an amount sufficient to maintain a plasma concentration of 3 ng/ml of the compound in a subject, over at least 28 days.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and/or a greater than or equal to 15% decrease in tumor attenuation. In some embodiments of any of the aspects disclosed herein, the effective amount is an amount sufficient to maintain a plasma concentration of 3 ng/ml of the compound in a subject, over at least 14 days. In some embodiments of any of the aspects disclosed herein, the effective amount is an amount sufficient to maintain a plasma concentration of 3 ng/ml of the compound in a subject, over at least 21 days. In some embodiments of any of the aspects disclosed herein, the effective amount is an amount sufficient to maintain a plasma concentration of 3 ng/ml of the compound in a subject, over at least 28 days.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to maintain a plasma concentration of 3 ng/ml of the compound in a subject. In some embodiments of any of the aspects disclosed herein, the effective amount is an amount sufficient to maintain a plasma concentration of 3 ng/ml of the compound in a subject, over at least 14 days. In some embodiments of any of the aspects disclosed herein, the effective amount is an amount sufficient to maintain a plasma concentration of 3 ng/mL of the compound in a subject, over at least 21 days. In some embodiments of any of the aspects disclosed herein, the effective amount is an amount sufficient to maintain a plasma concentration of 3 ng/mL of the compound in a subject, over at least 28 days. Advantageously, a BRD9 inhibitor (e.g., compound 1 or a pharmaceutically acceptable salt thereof) may exhibit prolonged efficacy at BRD9 degradation, thus allowing for intermittent dosing regimens. For example, a BRD9 inhibitor (e.g., compound 1 or a pharmaceutically acceptable salt thereof) may be administered to the subject in need thereof twice weekly or less frequently (e.g., twice weekly to once bimonthly, twice weekly to once monthly, twice weekly to once biweekly, once weekly to once monthly, or once weekly to once biweekly; e.g., once weekly, once biweekly, once every three weeks, or once monthly).

A BRD9 inhibitor (e.g., compound 1 or a pharmaceutically acceptable salt thereof) may be administered in cycles (e.g., four- to eight-week-long cycles; e.g., four-week-long, six-week-long, or eight-week-long cycles). In some variants, the regimen may include once weekly dosages of an effective amount of a BRD9 inhibitor (e.g., compound 1 or a pharmaceutically acceptable salt thereof). For example, a total of two to three once weekly dosages may be administered per cycle, e.g., an effective amount of a BRD9 inhibitor (e.g., compound 1 or a pharmaceutically acceptable salt thereof) is administered on weeks 1 and 2 of the cycle, or an effective amount of a BRD9 inhibitor (e.g., compound 1 or a pharmaceutically acceptable salt thereof) is administered on weeks 1, 2, and 3 of the cycle. For example, a once weekly dosing may be implemented as follows: the first dose of an effective amount of a BRD9 inhibitor (e.g., compound 1 or a pharmaceutically acceptable salt thereof) may be administered on Day 1 of the cycle, and the second once weekly dose may be administered on Day 8 or 9 (preferably, Day 8) of the cycle. If a third dose is administration, the third dose may be administered on Day 15 or Day 16 (preferably, Day 15) of the cycle. Alternatively, the regimen may include once biweekly, once every three weeks, once monthly, or once bimonthly dosages of an effective amount of a BRD9 inhibitor (e.g., compound 1 or a pharmaceutically acceptable salt thereof). Typically, the first dose of a BRD9 inhibitor (e.g., compound 1 or a pharmaceutically acceptable salt thereof) is administered on week 1 of the cycle.

### Kits

The invention also features kits including (a) a pharmaceutical composition including an agent that reduces the level and/or activity of BRD9 in a cell or subject described herein, and (b) a package insert with instructions to perform any of the methods described herein. In some embodiments, the kit includes (a) a pharmaceutical composition including an agent that reduces the level and/or activity of BRD9 in a cell or subject described herein, (b) an additional therapeutic agent (e.g., an anti-cancer agent), and (c) a package insert with instructions to perform any of the methods described herein.

The following Examples are illustrative only and not intended to limit the invention in any way.

### EXAMPLES

### Example 1. BRD9 Degrader Causes Range of Effects Against Prostate Cell Line Xenograft Tumor Models In Vivo

Procedure: The experimental details of the studies, including tumor model, mouse strain, cell number implanted, vehicle used, days of treatment, treatment details, and average tumor size at start of dosing are outlined in Table 1. All study mice were purchased from Beijing Anikeeper Biotech and Matrigel was utilized as a basement membrane for all xenograft models. All xenografts were engrafted subcutaneously in the right flank region. The vehicles and Compound 1 (3 mg/kg) were administered twice weekly (BIW) intraperitoneally and enzalutamide (30 mg/kg) was administered orally once daily (QD), when applicable. Tumor volumes and body weights were measured over the course of study, and doses were adjusted by body weight to achieve the proper dose in terms of mg/kg. Animals were sacrificed on the final day of treatment as indicated in Table 1.

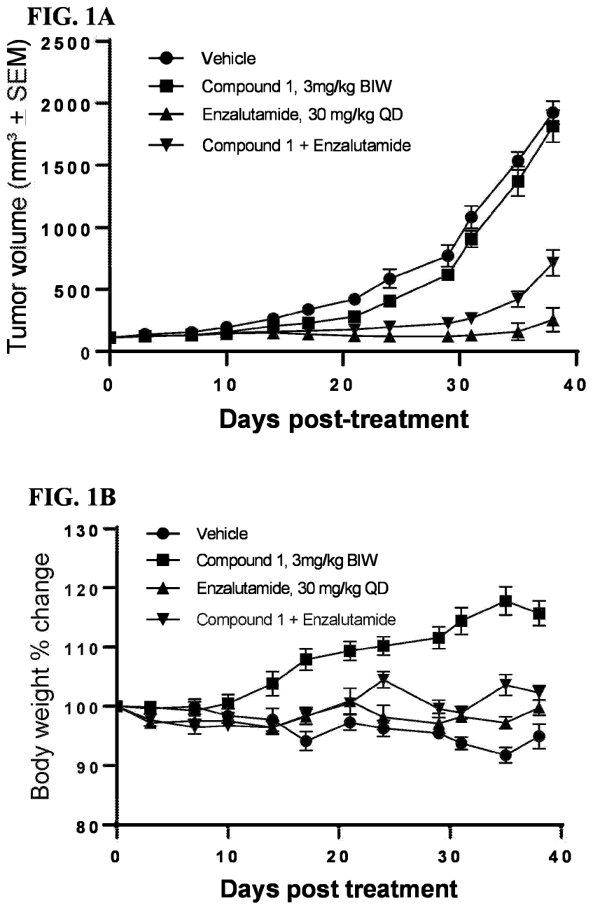

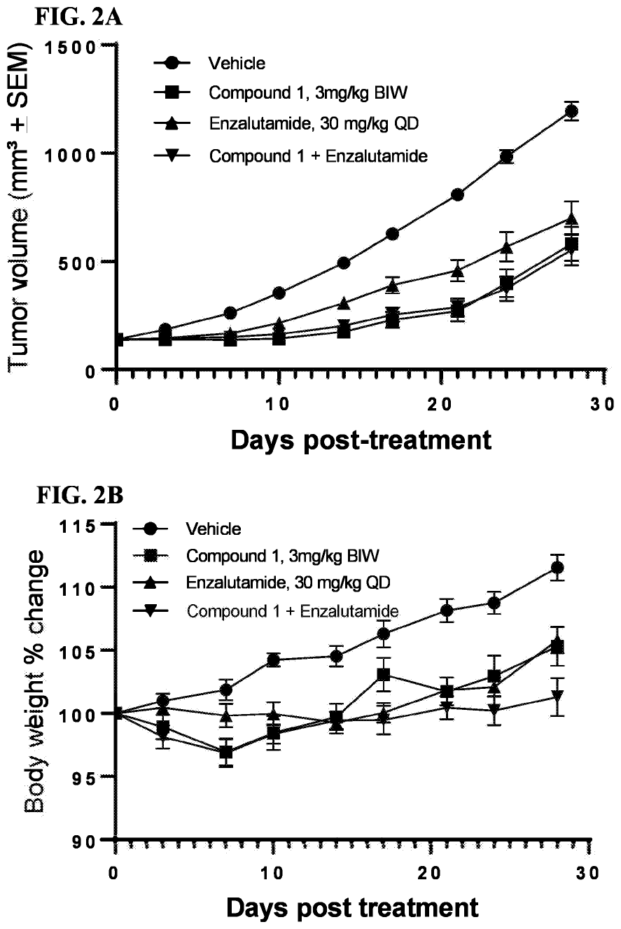

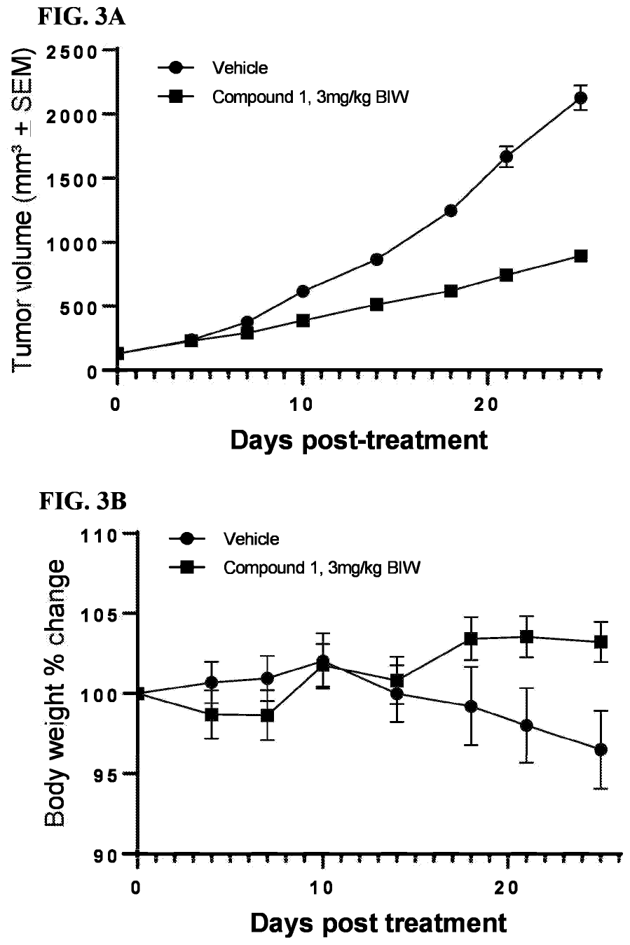

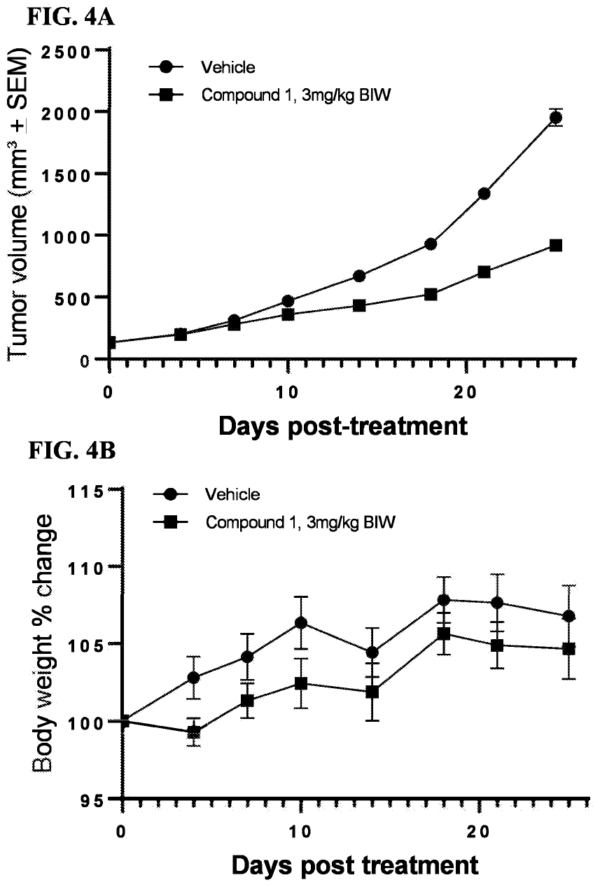

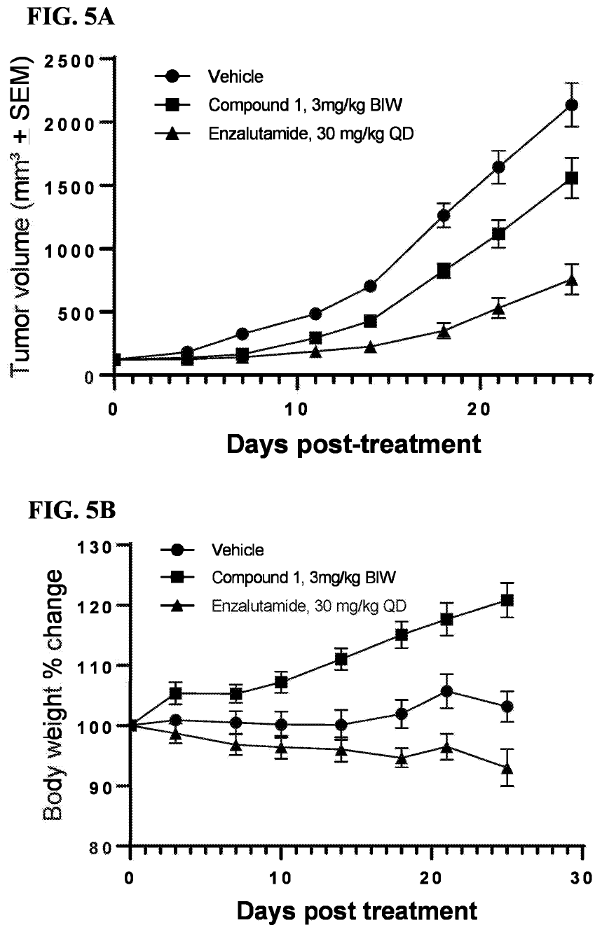

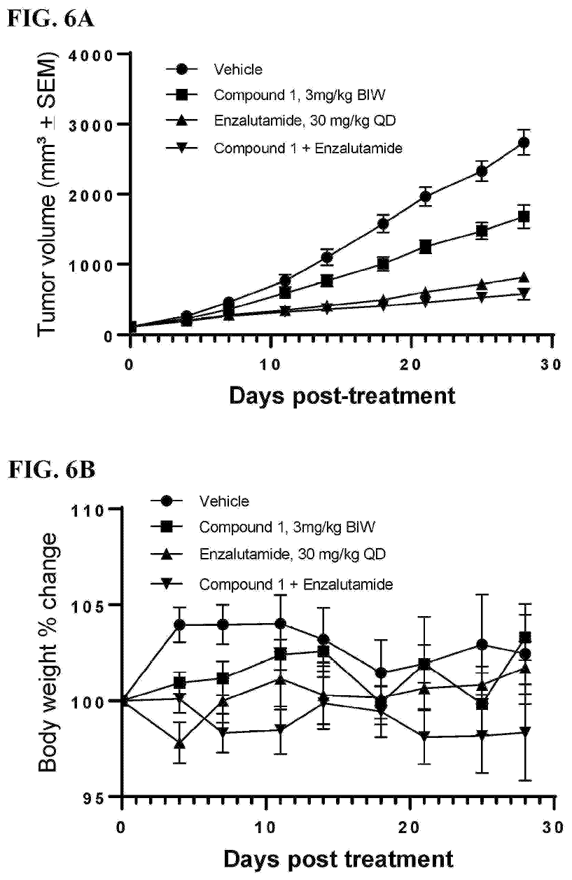

Results: Treatment with Compound 1 lead to a range of outcomes across the prostate xenograft models. Treatments in all models were well tolerated based on % body weight change observed (FIGS. 1B, 2B, 3B, 4B, 5B, 6B). Compound 1 treatment did not result in tumor growth inhibition (TGI) in the LNCaP xenograft model, however enzalutamide treatment did lead to tumor growth inhibition in this model. (FIG. 1A). In the VCaP xenograft model, treatment with Compound 1 lead to tumor growth inhibition that was greater than the inhibition provided by enzalutamide treatment. The combination did not show any additional tumor growth inhibition beyond that of single agent Compound 1. (FIG. 2A). Similar anti-tumor effect was observed after treatment of Compound 1 in prostate xenograft models PC3 (FIG. 3A) and NCI-H660 (FIG. 4A). In xenograft model C42B, Compound 1 treatment resulted in modest tumor growth inhibition which was less than the TGI provided by enzalutamide treatment. (FIG. 5A). In the prostate xenograft model 22RV1, Compound 1 treatment resulted in modest tumor growth inhibition which was less than the TGI provided by enzalutamide treatment. The combination did not show any additional tumor growth inhibition beyond that of single agent enzalutamide. (FIG. 6A).

The androgen receptor (AR) status and TGI values for Compound 1 treatments across all models can be found Table 2. Anti-androgen therapies, such as enzalutamide, are part of current standard of care (SOC) for androgen-dependent prostate cancers. However, cancers bearing the V7 splice variant of AR have been shown to be resistant to anti-androgen therapies. The anti-tumor effect demonstrated by Compound 1 treatment (see Table 2, TGI), especially in models that are either androgen-independent (AR-) or predicted to be resistant to current SOC anti-androgen therapies (e.g., AR-V7+), demonstrates that BRD9 degradation by Compound 1 can be a successful method by which to treat these tumors.

TABLE 1
Study details for cell line derived xenograft (CDX) models described in Example 1

| | | | | | | Average |
|---|---|---|---|---|---|---|
| | | Cell | | | | tumor size at |
| | | number | | Days of | | start of |
| Model | Strain | (millions) | Vehicle | treatment | Treatments | dosing (mm3) |
| LNCaP | NOD/SCID | 10 | 20% | 39 | Compound 1, | 114 |
| | | | sulfobutylether- | | enzalutamide, | |
| | | | β-cyclodextrin | | combination | |
| | | | (SBECD) | | | |
| VCaP | BALB/c | 10 | 20% (2- | 35 | Compound 1, | 138 |
| | nude | | Hydroxypropyl)- | | enzalutamide, | |
| | | | β-cyclodextrin | | combination | |
| | | | (HPBCD) | | | |
| PC3 | BALB/c | 5 | 20% HPBCD | 25 | Compound 1 | 130 |
| | nude | | | | | |
| NCI-H660 | BALB/c | 10 | 20% HPBCD | 25 | Compound 1 | 133 |
| | nude | | | | | |
| C42B | NOD/SCID | 5 | 20% HPBCD | 25 | Compound 1, | 123 |
| | | | | | enzalutamide | |
| 22RV1 | BALB/c | 5 | 20% SBECD | 43 | Compound 1, | 111 |
| | nude | | | | enzalutamide, | |
| | | | | | combination | |

TABLE 2
AR status and Compound 1-driven TGI values
for all CDX models described in Example 1.

| | | TGI with | TGI with |
|---|---|---|---|
| | | Compound 1 | Enzalutamide |
| Cell line | AR status | 3 mg/kg | 30 mg/kg |
| LNCaP | AR+ | 6.0% | 92.1% |
| VCaP | AR V7+ | 57.8% | 46.8% |
| PC3 | AR− | 61.7% | N/A |
| NCI-H660 | AR− | 56.9% | N/A |
| C42B | AR+ | 28.7% | 68.5% |
| 22RV1 | AR V7+ | 73.2% | 40.3% |

### Example 2. BRD9 Degrader Treatment Results in Tumor Growth Inhibition in an Enzalutamide-Resistant Model of Castration Resistant Prostate Cancer (CRPC)

The CDX model 22Rv1 has been previously described to express the V7 splice variant of AR and harbor resistance to enzalutamide treatment. Tumors expressing the AR-V7 variant have also been shown to have worse prognosis than those with wild type AR.

When treated with Compound 1, tumor growth of the CDX model 22Rv1 was inhibited (FIG. 6A). While enzalutamide was modestly active in this model (40.3% TGI), the anti-tumor effect of Compound 1 was greater (73.2% TGI) and the combination treatment of Compound 1 and enzalutamide was greater still (82.2% TGI) (Table 2).

### Example 3. BRD9 Degrader Treatment Results in Tumor Growth Inhibition in a Metastatic, AR-Independent CDX Model of CRPC

The PC3 model has been previously described as an androgen-independent model of CRPC Metastatic CRPC currently has no approved targeted therapies, with chemotherapy being the optimal treatment for androgen-independent CRPC.

When treated with Compound 1, tumor growth of the CDX model PC-3 was inhibited (FIG. 3A, TGI 61.7%).

### Example 4. BRD9 Degrader Treatment Results in Tumor Growth Inhibition in a CDX Model of Neuroendocrine Prostate Cancer (NEPC)

The CDX model NCI-H660 has been previously reported to have a molecular signature identifying it as a model of NEPC. Markers in this model include overexpression of Aurora kinase and N-Myc. Currently, there are no approved targeted therapies for subjects with NEPC.

When treated with Compound 1, tumor growth of the CDX model NCI-H660 was inhibited (FIG. 4A, TGI 56.9%).

### Example 5. BRD9 Degrader Treatment Results in Tumor Growth Inhibition in Castration Resistant, AR-Independent CDX Model DU145

Procedure: The experimental details of the studies, including tumor model, mouse strain, cell number implanted, vehicle used, days of treatment, treatment details, and average tumor size at start of dosing are outlined in Table 3. All study mice were obtained and the studies were conducted at Pharmaron. Each mouse was inoculated subcutaneously on the right flank with the single cell suspension of DU145 human prostate cancer tumor cells (5×10⁶) in 0.1 mL with Matrigel (1:1) in MEM with 10% FBS for tumor development.

Based on tumor volume, mice were randomly assigned to respective groups using a computer-generated randomization procedure. The study groups and the number of animals per group are shown in Table 4A (efficacy study) and Table 4B (PKPD study).

Treatment for the efficacy cohort was initiated when the mean tumor volume reached 145 mm³ on Day 7 post tumor cell inoculation. Animals for the efficacy study were randomized into 3 groups: 1) Vehicle Control BIW IP (administered twice per week, intraperitoneally (IP); 2) Compound 1 at 3 mg/kg BIW IP; and 3) Compound 1 at 15 mg/kg BIW IP.

Treatment for the PKPD cohort was initiated when the mean tumor volume reached 281 mm³ on Day 14 post tumor cell inoculation. Animals were randomized into 5 groups: 1) Vehicle Control, 3 doses intraperitoneally (IP); 2) Compound 1 at 1 mg/kg, single dose IP; 3) Compound 1 at 15 mg/kg, single dose IP; 4) Compound 1 at 1 mg/kg, 3 doses IP; and 5) Compound 1 at 15 mg/kg, 3 doses IP.

Animals that were observed to be in a continuing deteriorating condition or had a tumor size exceeding 2000 mm³ were euthanized prior to death, or before reaching a comatose state. In the case of an animal reaching a tumor size exceeding 2000 mm³, the entire group was terminated. Plasma and tumor samples were collected at 4 hours and 72 hours post last dose for all groups. For the PKPD Cohort, the tumors were cut into 2 pieces: one for Western Blotting (WB) with BRD9 analysis, and one for RNAseq analysis. For the Efficacy Cohort, the tumors were analyzed for BRD9 levels by Western Blotting (WB).

TABLE 3
Study details for cell line derived xenograft (CDX) model DU145

| | | Cell | | | | Average tumor |
|---|---|---|---|---|---|---|
| | | number | | Days of | | size at start of |
| Model | Strain | (millions) | Vehicle | treatment | Treatments | dosing (mm³) |
| DU145 | BALA/c | 5 | 20% (2- | 43 | Compound 1 | 145 (Efficacy) |
| | nude | | Hydroxypropyl)- | | | 281 (PKPD) |
| | | | β-cyclodextrin | | | |
| | | | (HPBCD) | | | |

TABLE 4A
Groups and Treatment for Efficacy Cohort

| | | Animals/ | Dose | Vol | | |
|---|---|---|---|---|---|---|
| Group # | Drug | group | (mg/kg) | (ml/kg) | Route | Frequency |
| 1 | Vehicle | 3 | — | 5 | IP | BIW |
| | control* | | | | | (43 days) |
| 2 | Compound 1 | 9 | 3 | 5 | IP | BIW |
| | | | | | | (43 days) |
| 3 | Compound 1 | 9 | 15 | 5 | IP | BIW |
| | | | | | | (43 days) |

*Vehicle Control = (20% HP-β-CD in 5 mM citrate buffer of pH 3), adjusted the final PH to 5 by the addition of 1N NaOH.

TABLE 4B
Groups and Treatment for PKPD Cohort

| | | Animals/ | Dose | Vol | | |
|---|---|---|---|---|---|---|
| Group # | Drug | group | (mg/kg) | (ml/kg) | Route | Frequency |
| 1 | Vehicle | 3 | — | 5 | IP | BIW (3 doses) |
| | control* | | | | | |
| 2 | Compound 1 | 6 | 1 | 5 | IP | Single dose |
| 3 | Compound 1 | 6 | 15 | 5 | IP | Single dose |
| 4 | Compound 1 | 6 | 1 | 5 | IP | BIW (3 doses) |
| 5 | Compound 1 | 6 | 15 | 5 | IP | BIW (3 doses) |

*Vehicle Control = (20% HP-β-CD in 5 mM citrate buffer of pH 3), adjusted the final PH to 5 by the addition of 1N NaOH.

### Results:

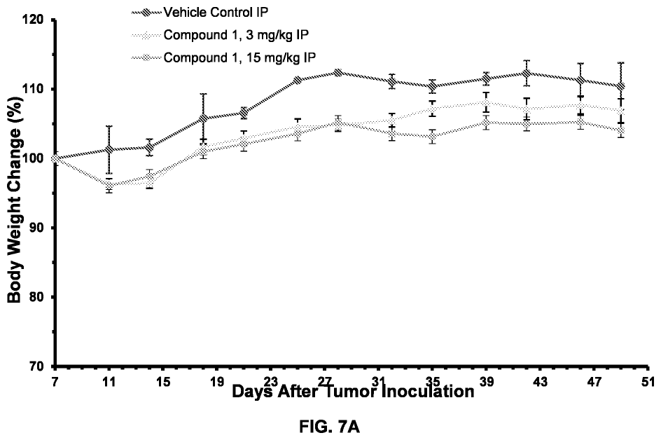

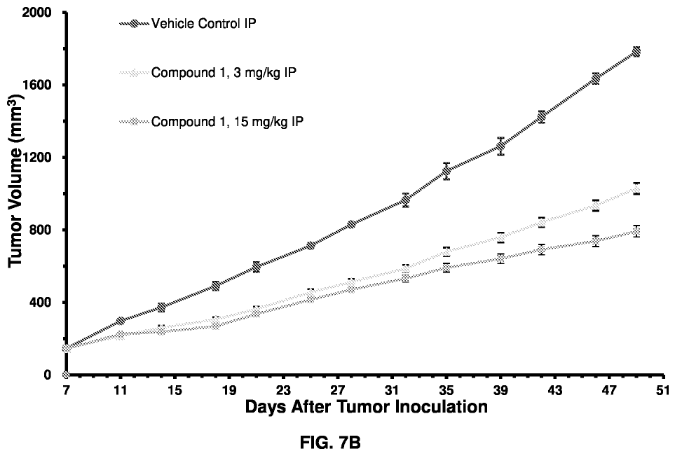

In the efficacy study, mean tumor volume (TV) of the vehicle group reached 1784 mm³ on Day 49 post tumor cell inoculation. Compound 1 at dose levels of 3 mg/kg and 15 mg/kg BIW showed significant antitumor activity with tumor growth inhibition (TGI) values of 42.2% and 55.3% (P<0.001 vs. vehicle control, Table 5, FIG. 7B), respectively. Regarding safety profile, the animals tolerated Compound 1 well at dose levels of 1 mg/kg, 3 mg/kg and 15 mg/kg during the treatment period. No obvious clinical abnormalities were observed on any of the animals treated. The growth curves of body weight for each study group over the entire course of study period are shown in FIG. 7A.

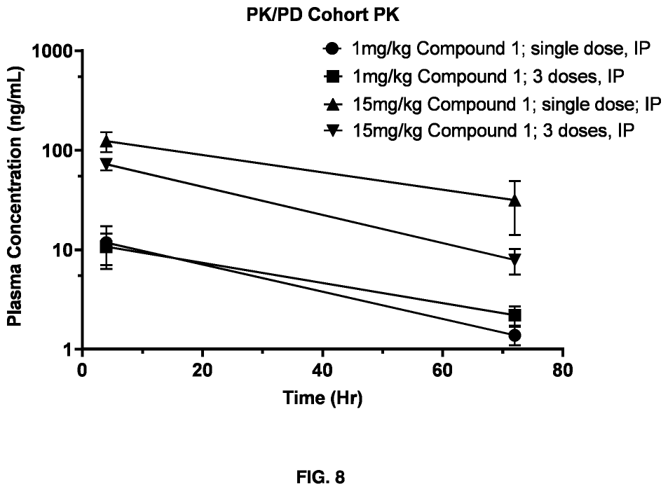

PK results illustrated that there were time and dose dependent exposures of Compound 1 in plasma. There was no obvious exposure difference between a single dose and three doses for Compound 1 at each dose level. The PK results of the test articles in plasma of different treatment groups are shown in Table 6A (4 hours) and Table 6B (72 hours). No difference in PK between a single dose or three doses of 1 mg/kg Compound 1 was observed (FIG. 8). Moreover, the 15 mg/kg (single dose) group had better PK exposure than the 15 mg/kg (three doses) group (FIG. 8).

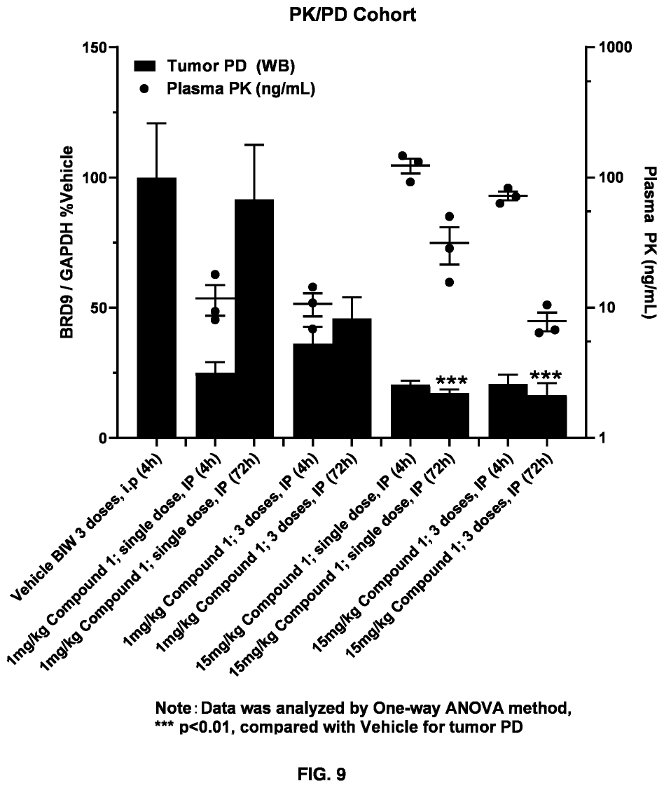

Western Blot (WB) results showed that PK/PD cohorts 15 mg/kg (single dose) and 15 mg/kg (three doses) of Compound 1 statistically and significantly degrade BRD9 protein in a DU145 AR negative prostate CDX model at 72 h post last dose (P<0.05 vs. vehicle control). Compound 1 plasma PK positively corelates with in vivo BRD9 degradation in DU145 AR-negative prostate CDX model (FIG. 9).

TABLE 5
Antitumor Activity of Different Treatment Groups
in DU145 Subcutaneous Model Efficacy Study

| | | Tumor | | | | |
|---|---|---|---|---|---|---|
| | | Size | Tumor | | | |
| Group | | (mm³) | Growth % | T/C* | TGI | P- |
| # | Treatment | on Day 49 | on Day 49 | (%) | (%) | Value |
| 1 | Vehicle | 1784 ± 25 | 1,228 ± 49 | — | — | — |
| 2 | 3 mg/kg of | 1029 ± 30 | 710 ± 19 | 57.8 | 42.2 | <0.001 |
| | Compound 1 | | | | | |
| 3 | 15 mg/kg of | 793 ± 31 | 549 ± 26 | 44.7 | 55.3 | <0.001 |
| | Compound 1 | | | | | |

*T/C = 100*(Vt relative volumes (% tumor growth)/VC relative volumes)

TABLE 6A
Plasma Concentration of Compound 1 in PK/PD Cohort at 4 Hours

| | Mouse | | Concentration | | | |
|---|---|---|---|---|---|---|
| Treatment | Identifier | | of Compound 1 | Mean | SD | CV |
| Group | # | Treatment | (ng/mL) | (ng/mL) | (ng/mL) | (%) |
| 2 | 11 | 1 mg/kg of | 8.07 | 11.8 | 5.4 | 45.5 |
| | | Compound 1, | | | | |
| | | single dose | | | | |
| 2 | 29 | 1 mg/kg of | 18.0 | | | |
| | | Compound 1, | | | | |
| | | single dose | | | | |
| 2 | 30 | 1 mg/kg of | 9.37 | | | |
| | | Compound 1, | | | | |
| | | single dose | | | | |
| 3 | 5 | 15 mg/kg of | 147 | 124 | 28 | 22.9 |
| | | Compound 1, | | | | |
| | | single dose | | | | |
| 3 | 9 | 15 mg/kg of | 132 | | | |
| | | Compound 1, | | | | |
| | | single dose | | | | |
| 3 | 12 | 15 mg/kg of | 92.3 | | | |
| | | Compound 1, | | | | |
| | | single dose | | | | |
| 4 | 8 | 1 mg/kg of | 10.9 | 10.8 | 3.8 | 35.1 |
| | | Compound 1, | | | | |
| | | BIW 3 doses | | | | |
| 4 | 20 | 1 mg/kg of | 14.4 | | | |
| | | Compound 1, | | | | |
| | | BIW 3 doses | | | | |
| 4 | 52 | 1 mg/kg of | 6.89 | | | |
| | | Compound 1, | | | | |
| | | BIW 3 doses | | | | |
| 5 | 16 | 15 mg/kg of | 82.9 | 72.5 | 9.8 | 13.5 |
| | | Compound 1, | | | | |
| | | BIW 3 doses | | | | |
| 5 | 37 | 15 mg/kg of | 63.4 | | | |
| | | Compound 1, | | | | |
| | | BIW 3 doses | | | | |
| 5 | 42 | 15 mg/kg of | 71.3 | | | |
| | | Compound 1, | | | | |
| | | BIW 3 doses | | | | |

TABLE 6B
Plasma Concentration of Compound 1 in PK/PD Cohort at 72 Hours

| | Mouse | | Concentration | | | |
|---|---|---|---|---|---|---|
| Treatment | Identifier | | of Compound 1 | Mean | SD | CV |
| Group | # | Treatment | (ng/mL) | (ng/mL) | (ng/mL) | (%) |
| 2 | 33 | 1 mg/kg of | BLOQ* | 1.39 | N/A | N/A |
| | | Compound 1, | | | | |
| | | single dose | | | | |
| 2 | 50 | 1 mg/kg of | 1.18 | | | |
| | | Compound 1, | | | | |
| | | single dose | | | | |
| 2 | 62 | 1 mg/kg of | 1.59 | | | |
| | | Compound 1, | | | | |
| | | single dose | | | | |
| 3 | 32 | 15 mg/kg of | 15.7 | 31.5 | 17.4 | 55.3 |
| | | Compound 1, | | | | |
| | | single dose | | | | |
| 3 | 45 | 15 mg/kg of | 50.2 | | | |
| | | Compound 1, | | | | |
| | | single dose | | | | |
| 3 | 63 | 15 mg/kg of | 28.6 | | | |
| | | Compound 1, | | | | |
| | | single dose | | | | |
| 4 | 55 | 1 mg/kg of | 2.02 | 2.21 | 0.48 | 21.7 |
| | | Compound 1, | | | | |
| | | BIW 3 doses | | | | |
| 4 | 59 | 1 mg/kg of | 2.75 | | | |
| | | Compound 1, | | | | |
| | | BIW 3 doses | | | | |
| 4 | 64 | 1 mg/kg of | 1.85 | | | |
| | | Compound 1, | | | | |
| | | BIW 3 doses | | | | |
| 5 | 14 | 15 mg/kg of | 6.43 | 7.9 | 2.3 | 28.5 |
| | | Compound 1, | | | | |
| | | BIW 3 doses | | | | |
| 5 | 39 | 15 mg/kg of | 10.5 | | | |
| | | Compound 1, | | | | |
| | | BIW 3 doses | | | | |
| 5 | 70 | 15 mg/kg of | 6.77 | | | |
| | | Compound 1, | | | | |
| | | BIW 3 doses | | | | |

*BLOQ = Below quantifiable limit of 1.05 ng/mL

### Example 6: BRD9 Degrader Treatment Results in Moderate Tumor Growth Inhibition in AR-Positive, CDX Model of CRPC (VCaP Model)

Procedure: The experimental details of the studies, including tumor model, mouse strain, cell number implanted, vehicle used, days of treatment, treatment details, and average tumor size at start of dosing are outlined in Table 7. All study mice were obtained and the studies were conducted at Pharmaron. Each mouse was inoculated subcutaneously on the right flank with the single cell suspension of VCAP human prostate cancer tumor cells (1×10⁷) in 0.1 mL with Matrigel (1:1) in DMEM medium with 10% FBS for tumor development. The mice were castrated by removing the testes via a scrotal approach when the tumor volume reached 270 mm³. After castration, the tumor volume decreased. Treatment started when the mean tumor volume grew back 265 mm³. Based on tumor volume, mice were randomly assigned to respective groups using a computer-generated randomization procedure and treated according to the study design in Table 8.

TABLE 7
Study details for cell line derived xenograft (CDX) model VCaP

| | | | | | | Average |
|---|---|---|---|---|---|---|
| | | | | | | tumor size |
| | | Cell | | | | at start of |
| | | number | | Days of | | dosing |
| Model | Strain | (millions) | Vehicle | treatment | Treatments | (mm3) |
| CRPC | NCG | 10 | 20% (2- | 25 | Compound 1 | 265 |
| VCaP | | | Hydroxypropyl)- | | alone; | |
| | | | β-cyclodextrin | | enzalutamide | |
| | | | (HPBCD) | | alone, and a | |
| | | | | | combination of | |
| | | | | | Compound 1 and | |
| | | | | | enzalutamide | |

TABLE 8
Study Groups and Treatments

| Group | | Animals/ | Dose | Vol | | | |
|---|---|---|---|---|---|---|---|
| # | Drug | group | (mg/kg) | (ml/kg) | Route | Frequency | Duration |
| 1 | Vehicle* | 8 | — | 5 | IP | BIW | 25 days |
| 2 | Compound 1 | 8 | 10 | 5 | IP | BIW | 25 days |
| 3 | Enzalutamide | 8 | 30 | 10 | PO | QD | 25 days |
| 4 | Compound 1 + | 8 | 10 | 5 | IP | BIW | 25 days |
| | Enzalutamide | | 30 | 10 | PO | QD | |

*Vehicle for Enzalutamide = 5% DMSO/95% (1% CMC/0.1% Tween80, v/v), v/v

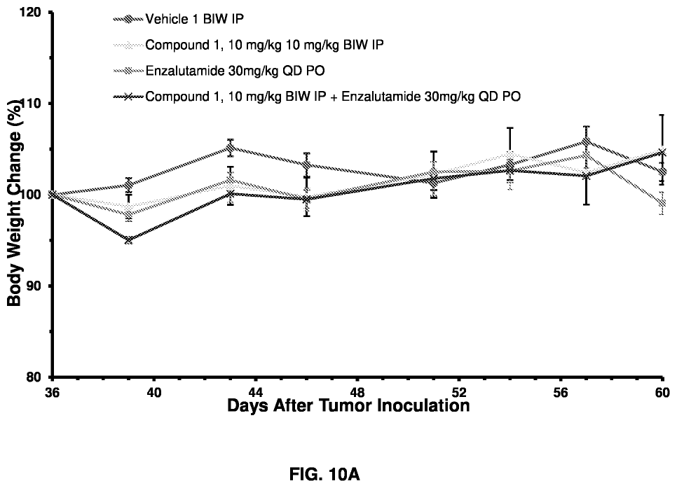

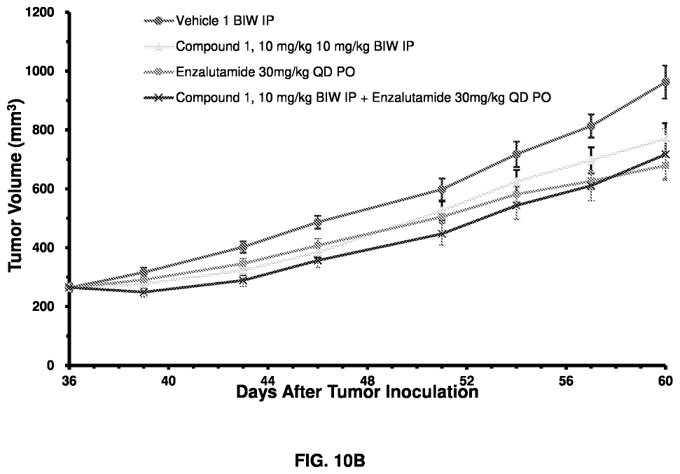

Results: The mean tumor size of vehicle treated mice reached 814 mm³ on day 57 after tumor inoculation. The treatments were initiated when mean tumor volume reached 265 mm³ on Day 36 post tumor inoculation. The inhibition rates of tumor growth are summarized in Table 9 and FIG. 10B. Compound 1 at dose level of 10 mg/kg showed minimal antitumor trend with TGI of 13.8%. Enzalutamide at dose level of 30 mg/kg showed moderate antitumor activity with TGI of 23.3% (P=0.001vs. vehicle group). Compound 1 at dose level of 10 mg/kg plus enzalutamide at dose level of 30 mg/kg showed moderate antitumor activity with TGI of 25.9% (P<0.001vs. vehicle group).

Regarding the safety profile, treatment was well tolerated, as body weight did not decrease significantly during the course of the study (FIG. 10A). One animal treated with Compound 1 at 10 mg/kg, and two animals treated with Compound 1 at 10 mg/kg +Enzalutamide at 30 mg/kg were found dead at the late stage of the treatment period. These male mice had been castrated by removing the testes via a scrotal approach when the tumor volume reached 270 mm³, thus, it is suspected that surgery may have led to these deaths. Additionally, no other clinical abnormalities were observed on all animals treated.

TABLE 9
Antitumor Activity of Different Treatment
Groups in VCAP CRPC Model

| | Tumor Size | Tumor Growth | | | |
|---|---|---|---|---|---|
| | (mm3) | % | T/C | TGI | |
| | on Day 57 | on Day 57 | (%) | (%) | P Value |
| G1: Vehicle | 814 ± 39 | 310 ± 10 | — | — | — |
| G2: Compound 1 | 697 ± 44 | 267 ± 20 | 86.2 | 13.8 | 0.028 |
| at 10 mg/kg | | | | | |
| G3: Enzalutamide | 626 ± 28 | 238 ± 9 | 76.7 | 23.3 | 0.001 |
| at 30 mg/kg | | | | | |
| G4: Compound 1 | 611 ± 51 | 230 ± 15 | 74.1 | 25.9 | <0.001 |
| at 10 mg/kg + | | | | | |
| Enzalutamide at | | | | | |
| 30 mg/kg | | | | | |

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

What is claimed is:

1. A method of treating androgen receptor-independent prostate cancer in a subject in need thereof, the method comprising administering to the subject an effective amount of a compound that reduces the level and/or activity of BRD9, or a pharmaceutically acceptable salt thereof.

2. A method of slowing progression of androgen receptor-independent prostate cancer in a subject in need thereof, the method comprising administering to the subject an effective amount of a compound that reduces the level and/or activity of BRD9, or a pharmaceutically acceptable salt thereof.

3. A method of reducing recurrence of androgen receptor-independent prostate cancer in a subject in need thereof, the method comprising administering to the subject an effective amount of a compound that reduces the level and/or activity of BRD9, or a pharmaceutically acceptable salt thereof.

4. A method of decreasing the rate of metastatic tumor seeding of androgen receptor-independent prostate cancer in a subject in need thereof, the method comprising administering to the subject an effective amount of a compound that reduces the level and/or activity of BRD9, or a pharmaceutically acceptable salt thereof.

5. A method of decreasing metastatic tumor nodule formation of androgen receptor-independent prostate cancer in a subject in need thereof, the method comprising administering to the subject an effective amount of a compound that reduces the level and/or activity of BRD9, or a pharmaceutically acceptable salt thereof.

6. A method of decreasing the spread of metastatic tumor nodule formation of androgen receptor-independent prostate cancer in a subject in need thereof, the method comprising administering to the subject an effective amount of a compound that reduces the level and/or activity of BRD9, or a pharmaceutically acceptable salt thereof.

7. A method of decreasing metastatic colonization of androgen receptor-independent prostate cancer in a subject in need thereof, the method comprising administering to the subject an effective amount of a compound that reduces the level and/or activity of BRD9, or a pharmaceutically acceptable salt thereof.

8. The method of any one of claims 1-7, wherein the androgen receptor-independent prostate cancer has failed to respond a previous treatment with an anti-cancer therapy.

9. A method of treating prostate cancer that has failed to respond to a previous treatment with an anti-cancer therapy in a subject in need thereof, the method comprising administering to the subject an effective amount of a compound that reduces the level and/or activity of BRD9, or a pharmaceutically acceptable salt thereof.

10. The method of any one of claims 1-9, wherein the prostate cancer is neuroendocrine prostate cancer.

11. A method of treating neuroendocrine prostate cancer in a subject in need thereof, the method comprising administering to the subject an effective amount of a compound that reduces the level and/or activity of BRD9, or a pharmaceutically acceptable salt thereof.

12. The method of claim 11, wherein the neuroendocrine prostate cancer is androgen receptor-independent.

13. The method of claim 11 or 12, wherein the neuroendocrine prostate cancer has failed to respond to a previous treatment with an anti-cancer therapy.

14. The method of any one of claims 1 to 13, wherein the compound is 3-(6-(7-((1-(4-(6-(azetidin-1-yl)-2-methyl-1-oxo-1,2-dihydro-2,7-naphthyridin-4-yl)-2,6-dimethoxybenzyl) piperidin-4-yl)methyl)-2,7-diazaspiro[3.5]nonan-2-yl)-1-oxoisoindolin-2-yl) piperidine-2,6-dione or a pharmaceutically acceptable salt thereof.

15. The method of any one of claims 1 to 13, wherein the compound is 3-((4-(4-(1-(2,6-dimethoxy-4-(1,4,5-trimethyl-6-oxo-1,6-dihydropyridin-3-yl)benzyl)-3,3-difluoropiperidin-4-yl) piperazin-1-yl)-3-fluorophenyl)amino) piperidine-2,6-dione or a pharmaceutically acceptable salt thereof.

16. The method of any one of claims 1 to 15, wherein the prostate cancer is castration-resistant prostate cancer (CRPC).

17. The method of any one of claims 1 to 16, wherein the prostate cancer is small cell prostate cancer.

18. The method of claim 2, wherein the effective amount is an amount sufficient to reduce the level of neuroendocrine prostate cancer cells in the subject compared to a subject that is not administered the compound or pharmaceutically acceptable salt thereof.

19. The method of any one of claims 1 to 18, wherein the anti-cancer therapy is active surveillance, surgery, radiation therapy, high-intensity focused ultrasound (HIFU), cryotherapy, hormone therapy, chemotherapy, immunotherapy, vaccine treatment, immune checkpoint inhibitors, targeted therapy drugs, or bone-directed treatment.

20. The method of any one of claims 1 to 19, wherein the anti-cancer therapy is abiraterone acetate, alendronate, apalutamide, bicalutamide, cabazitaxel, carboplatin, cisplatin, darolutamide, degarelix, denosumab, docetaxel, enzalutamide, etoposide, flutamide, goserelin acetate, ibandronate, leuprolide acetate, lynparza, mitoxantrone hydrochloride, nilutamide, olaparib, pamidronate, radium 223 dichloride, relugolix, risedronate, rucaparib camsylate, sipuleucel-T, or zoledronic acid, or combinations thereof.

21. The method of claim 20, wherein the anti-cancer therapy is enzalutamide.

22. The method of any one of claims 1 to 21, wherein the subject is further administered at least one additional anti-cancer therapy.

23. The method of claim 22, wherein the additional anti-cancer therapy is administered prior to the administering of the compound or pharmaceutically acceptable salt thereof.

24. The method of claim 22, wherein the additional anti-cancer therapy is administered in addition to the administering of the compound or pharmaceutically acceptable salt thereof.

25. The method of claim 22, wherein the additional anti-cancer therapy is administered subsequent to the administering of the compound or pharmaceutically acceptable salt thereof.

26. The method of any one of the preceding claims, wherein the subject is further administered a treatment for symptoms of prostate cancer.

27. The method of claim 26, wherein the further treatment is prednisone, methylprednisolone, pembrolizumab, or a combination thereof.

28. The method of any one of the preceding claims, wherein the effective amount is an amount sufficient to reduce the level of luminal prostate cancer cell to neuroendocrine prostate cancer cell trans-differentiation measured by lower expression levels of CHGA, SYP, and/or ENO2 compared to a subject that is not administered the compound or pharmaceutically acceptable salt thereof.

29. The method of any one of the preceding claims, wherein the effective amount is an amount sufficient to reduce the level of adenocarcinoma to neuroendocrine trans-differentiation measured by lower expression levels of CHGA, SYP, and/or ENO2 compared to a subject that is not administered the compound or pharmaceutically acceptable salt thereof.

30. The method of any one of the preceding claims, wherein the effective amount is an amount sufficient to reduce the level of neuroendocrine prostate cancer cells compared to a subject that is not administered the compound or pharmaceutically acceptable salt thereof.

31. The method of any one of the preceding claims, wherein the prostate cancer has been determined to have or predicted to have lower expression levels of AR, KLK2, KLK3, CDH1, CYLD, NKX3-1, SLC45A3, TARP, PTEN, SPDEF, TP53, or RB1, or combinations thereof compared to a subject that does not have prostate cancer.

32. The method of any one of the preceding claims, wherein the prostate cancer has been determined to have or predicted to have lower expression levels of AR, KLK2, KLK3, CDH1, CYLD, NKX3-1, SLC45A3, TARP, PTEN, SPDEF, TP53, or RB1, or combinations thereof compared to standard levels for prostate cancer.

33. The method of any one of the preceding claims, wherein the prostate cancer has been determined to have or predicted to have higher expression levels of CHGB, CHGA, SYP, ENO2, PEG10, SNAP25, SRRM4, VGF, VIM, SCGN, PAPPA2, or WNT11, or combinations thereof compared to a subject that does not have prostate cancer.

34. The method of any one of the preceding claims, wherein the prostate cancer has been determined to have or predicted to have higher expression levels of CHGB, CHGA, SYP, ENO2, PEG10, SNAP25, SRRM4, VGF, VIM, SCGN, PAPPA2, or WNT11, or combinations thereof compared to standard levels for prostate cancer.

35. The method of any one of the preceding claims, wherein the prostate cancer has been determined to have or predicted to have higher expression levels of ASCL1, EZH2, DLX5, DLX6, SOX2, NKX2-2, HES6, SOX9, KDM3A, FOXA2, AURKA, MYCN, MYC, AKT, POU3F2/BRN2, NANOG, ONECUT2, or NKX2-1, or combinations thereof compared to a subject that does not have prostate cancer.

36. The method of any one of the preceding claims, wherein the prostate cancer has been determined to have or predicted to have higher expression levels of ASCL1, EZH2, DLX5, DLX6, SOX2, NKX2-2, HES6, SOX9, KDM3A, FOXA2, AURKA, MYCN, MYC, AKT, POU3F2/BRN2, NANOG, ONECUT2, or NKX2-1, or combinations thereof compared to standard levels for prostate cancer.

37. The method of any one of the preceding claims, wherein the prostate cancer has been determined to have or predicted to have lower expression levels of RE1 silencing transcription factor (REST).

38. The method of any one of the preceding claims, wherein the prostate cancer has been determined to have or predicted to have expression of AR and KLK3 (PSA).

39. The method of any one of the preceding claims, wherein the prostate cancer has been determined to have or predicted to have undetectable expression of CHGA and SYP.

40. The method of any one of the preceding claims, wherein the prostate cancer has been determined to have or predicted to have expression of AR, KLK3 (PSA), CHGA and SYP.

41. The method of any one of the preceding claims, wherein the prostate cancer has been determined to have or predicted to have undetectable expression of AR and KLK3 (PSA).

42. The method of any one of the preceding claims, wherein the prostate cancer has been determined to have or predicted to have expression of CHGA and SYP.

43. The method of any one of the preceding claims, wherein the prostate cancer has been determined to have or predicted to have undetectable expression of AR, KLK3 (PSA), CHGA and SYP.

44. The method of any one of the preceding claims, wherein the prostate cancer has been determined or predicted to have a high level of TMPRSS2-ERG fusions.

45. The method of any one of the preceding claims, wherein the prostate cancer is metastatic.

46. The method of any one of the preceding claims, wherein expression of BRD9, GLTSCR1, CXXC5 or TET2 is increased in the prostate cancer compared to a subject that does not have prostate cancer.

47. The method of claim 46, wherein expression of BRD9 is increased in the prostate cancer compared to a subject that does not have prostate cancer.

48. The method of claim 46, wherein expression of GLTSCR1 is increased in the prostate cancer compared to a subject that does not have prostate cancer.

49. The method of any one of the preceding claims, wherein expression of TET2, CXXC5, H3K27ac, ID1, PFN2, or ID3 in the subject is increased in the prostate cancer determined to or predicted to be resistant to enzalutamide compared to a prostate cancer that responds to treatment with enzalutamide.

50. The method of any one of the preceding claims, wherein the prostate cancer has been determined to have or predicted to have undetectable expression of PTEN.

51. The method of any one of the preceding claims, wherein the prostate cancer has been determined or predicted to be ERG positive.

52. The method of any one of the preceding claims, wherein the subject is further administered an inhibitor or degrader of ERG.

53. The method of claim 52, wherein the ERG inhibitor or degrader is ERGi-USU (1-[2-Thiazolylazo]-2-naphthol).

54. The method of any one of the preceding claims, wherein the subject is further administered a degrader of AR.

55. The method of claim 54, wherein the AR degrader is bavdegalutamide (ARV-110), ARV-766, or AR-V7.

56. The method of any one of the preceding claims, wherein the subject is further administered an inhibitor of the JAK-STAT pathway.

57. The method of claim 56, wherein the JAK-STAT inhibitor is AG490, AZD1480, AZD4205, baricitinib, dasatinib, fedratinib, filgotinib, itacitnib, lestaurtinib, momelotinib, pacritinib, peficitinib, ruxolitinib, siltuximab, tofacitinib, upadacitinib, or WP1066.

58. The method of any one of the preceding claims, wherein the subject is further administered an inhibitor of the MAPK pathway.

59. The method of claim 58, wherein the MAPK pathway inhibitor is a Farnesyltransferase inhibitor (FTI), Sorafenib, Vemurafenib, PLX8394, Dabrafenib, Ulixertinib, Simvastatin, Alisertib, or Teriflunomide.

60. The method of any one of the preceding claims, wherein the subject is further administered an inhibitor of the PI3K-AKT-mTOR pathway.

61. The method of claim 60, wherein the PIK3-AKT-mTOR inhibitor is everolimus, alpelisib, idelalisib or copanlisib.

62. The method of any one of the preceding claims, wherein the subject has a PSA level of 4 ng/ml or more prior to the administering of the compound or a pharmaceutically acceptable salt thereof.

63. The method of any one of claims 1-62, wherein the effective amount of the compound or a pharmaceutically acceptable salt thereof, is administered in a dose of 20-80 mg/kg.

64. The method of any one of claims 1-62, wherein the effective amount of the compound or a pharmaceutically acceptable salt thereof, is administered in a dose of 20-60 mg/kg.

65. The method of any one of claims 1-62, wherein the effective amount of the compound or a pharmaceutically acceptable salt thereof, is administered in a dose of 20-40 mg/kg.

66. The method of any one of claims 1-62, wherein the effective amount of the compound or a pharmaceutically acceptable salt thereof, is administered in a dose of 20 mg/kg.

67. The method of any one of claims 1-62, wherein the effective amount of the compound or a pharmaceutically acceptable salt thereof, is administered in a dose of 40 mg/kg.

68. The method of any one of claims 1-62, wherein the effective amount of the compound or a pharmaceutically acceptable salt thereof, is administered in a dose of 50 mg/kg.

69. The method of any one of claims 1-62, wherein the effective amount of the compound or a pharmaceutically acceptable salt thereof, is administered in a dose of 60 mg/kg.

70. The method of any one of claims 1-62, wherein the effective amount of the compound or a pharmaceutically acceptable salt thereof, is administered in a dose of 80 mg/kg.

71. The method of any one of claims 1-62, wherein the effective amount of the compound or a pharmaceutically acceptable salt thereof, is administered in a dose of 120 mg/kg.

72. The method of any one of claims 1-62, wherein the effective amount of the compound or a pharmaceutically acceptable salt thereof, is administered to the subject at least once per week.

73. The method of any one of claims 1-62, wherein the effective amount of the compound or a pharmaceutically acceptable salt thereof, is administered to the subject at least twice per week.

74. The method of any one of claims 1-62, wherein the effective amount of the compound or a pharmaceutically acceptable salt thereof, is administered in a dose of 20 mg/kg once per week.

75. The method of any one of claims 1-62, wherein the effective amount of the compound or a pharmaceutically acceptable salt thereof, is administered in a dose of 20 mg/kg twice per week.

76. The method of any one of claims 1-62, wherein the effective amount of the compound or a pharmaceutically acceptable salt thereof, is administered in a dose of 40 mg/kg once per week.

77. The method of any one of claims 1-62, wherein the effective amount of the compound or a pharmaceutically acceptable salt thereof, is administered in a dose of 40 mg/kg twice per week.

78. The method of any one of claims 1-62, wherein the effective amount of the compound or a pharmaceutically acceptable salt thereof, is administered in a dose of 50 mg/kg once per week.

79. The method of any one of claims 1-62, wherein the effective amount of the compound or a pharmaceutically acceptable salt thereof, is administered in a dose of 50 mg/kg twice per week.

80. The method of any one of claims 1-62, wherein the effective amount of the compound or a pharmaceutically acceptable salt thereof, is administered in a dose of 60 mg/kg once per week.

81. The method of any one of claims 1-62, wherein the effective amount of the compound or a pharmaceutically acceptable salt thereof, is administered in a dose of 60 mg/kg twice per week.

82. The method of any one of claims 1-62, wherein the effective amount of the compound or a pharmaceutically acceptable salt thereof, is administered in a dose of 80 mg/kg once per week.

83. The method of any one of claims 1-62, wherein the effective amount of the compound or a pharmaceutically acceptable salt thereof, is administered in a dose of 80 mg/kg twice per week.

84. The method of any one of claims 1-62, wherein the effective amount of the compound or a pharmaceutically acceptable salt thereof, is administered in a dose of 120 mg/kg once per week.

85. The method of any one of claims 1-62, wherein the effective amount of the compound or a pharmaceutically acceptable salt thereof, is administered in a dose of 120 mg/kg twice per week.

86. The method of any one of claims 1-85, wherein the compound or a pharmaceutically acceptable salt thereof is administered to the subject in a 14-day dosing cycle.

87. The method of any one of claims 1-86, wherein the compound or a pharmaceutically acceptable salt thereof is administered to the subject in a 21-day dosing cycle.

88. The method of any one of claims 1-87, wherein the compound or a pharmaceutically acceptable salt thereof is administered to the subject in a 28-day dosing cycle.

89. The method of any one of the preceding claims, wherein the compound or a pharmaceutically acceptable salt thereof, is administered to the subject intravenously.

90. The method of any one of the preceding claims, wherein the compound or a pharmaceutically acceptable salt thereof, is administered to the subject subcutaneously.

91. The method of any one of the preceding claims, wherein the compound or a pharmaceutically acceptable salt thereof, is administered to the subject intramuscularly.


SEQUENCE LISTING (2 sequences)

SEQ ID NO: 1 - protein, 1794 aa, Homo sapiens
ATGGGCAAGAAGCACAAGAAGCACAAGGCCGAGTGGCGCTCGTCCTACGAGGATTATGCCGACAAGCCCCTGGAGAAGCCTCTAAAGCTAGTCCTGAAGGTCGGAGGAAGTGAAGTGACTGAACTCTCAGGATCCGGCCACGACTCCAGTTACTATGATGACAGGTCAGACCATGAGCGAGAGAGGCACAAAGAAAAGAAAAAGAAGAAGAAGAAGAAGTCCGAGAAGGAGAAGCATCTGGACGATGAGGAAAGAAGGAAGCGAAAGGAAGAGAAGAAGCGGAAGCGAGAGAGGGAGCACTGTGACACGGAGGGAGAGGCTGACGACTTTGATCCTGGGAAGAAGGTGGAGGTGGAGCCGCCCCCAGATCGGCCAGTCCGAGCGTGCCGGACACAGCCAGCCGAAAATGAGAGCACACCTATTCAGCAACTCCTGGAACACTTCCTCCGCCAGCTTCAGAGAAAAGATCCCCATGGATTTTTTGCTTTTCCTGTCACGGATGCAATTGCTCCTGGATATTCAATGATAATAAAACATCCCATGGATTTTGGCACCATGAAAGACAAAATTGTAGCTAATGAATACAAGTCAGTTACGGAATTTAAGGCAGATTTCAAGCTGATGTGTGATAATGCAATGACATACAATAGGCCAGATACCGTGTACTACAAGTTGGCGAAGAAGATCCTTCACGCAGGCTTTAAGATGATGAGCAAACAGGCAGCTCTTTTGGGCAATGAAGATACAGCTGTTGAGGAACCTGTCCCTGAAGTTGTACCAGTACAAGTAGAAACTGCCAAGAAATCCAAAAAGCCGAGTAGAGAAGTTATCAGCTGCATGTTTGAGCCTGAAGGGAATGCCTGCAGCTTGACGGACAGTACCGCAGAGGAGCACGTGCTGGCGCTGGTGGAGCACGCAGCTGACGAAGCTCGGGACAGGATCAACCGGTTCCTCCCAGGCGGCAAGATGGGCTATCTGAAGAGGAACGGGGACGGGAGCCTGCTCTACAGCGTGGTCAACACGGCCGAGCCGGACGCTGATGAGGAGGAGACCCACCCGGTGGACTTGAGCTCGCTCTCCAGTAAGCTACTCCCAGGCTTCACCACGCTGGGCTTCAAAGACGAGAGAAGAAACAAAGTCACCTTTCTCTCCAGTGCCACTACTGCGCTTTCGATGCAGAATAATTCAGTATTTGGCGACTTGAAGTCGGACGAGATGGAGCTGCTCTACTCAGCCTACGGAGATGAGACAGGCGTGCAGTGTGCGCTGAGCCTGCAGGAGTTTGTGAAGGATGCTGGGAGCTACAGCAAGAAAGTGGTGGACGACCTCCTGGACCAGATCACAGGCGGAGACCACTCTAGGACGCTCTTCCAGCTGAAGCAGAGAAGAAATGTTCCCATGAAGCCTCCAGATGAAGCCAAGGTTGGGGACACCCTAGGAGACAGCAGCAGCTCTGTTCTGGAGTTCATGTCGATGAAGTCCTATCCCGACGTTTCTGTGGATATCTCCATGCTCAGCTCTCTGGGGAAGGTGAAGAAGGAGCTGGACCCTGACGACAGCCATTTGAACTTGGATGAGACGACGAAGCTCCTGCAGGACCTGCACGAAGCACAGGCGGAGCGCGGCGGCTCTCGGCCGTCGTCCAACCTCAGCTCCCTGTCCAACGCCTCCGAGAGGGACCAGCACCACCTGGGAAGCCCTTCTCGCCTGAGTGTCGGGGAGCAGCCAGACGTCACCCACGACCCCTATGAGTTTCTTCAGTCTCCAGAGCCTGCGGCCTCTGCCAAGACCTAA

SEQ ID NO: 2 - protein, 597 aa, Homo sapiens
MGKKHKKHKAEWRSSYEDYADKPLEKPLKLVLKVGGSEVTELSGSGHDSSYYDDRSDHERERHKEKKKKKKKKSEKEKHLDDEERRKRKEEKKRKREREHCDTEGEADDFDPGKKVEVEPPPDRPVRACRTQPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSKQAALLGNEDTAVEEPVPEVVPVQVETAKKSKKPSREVISCMFEPEGNACSLTDSTAEEHVLALVEHAADEARDRINRFLPGGKMGYLKRNGDGSLLYSVVNTAEPDADEEETHPVDLSSLSSKLLPGFTTLGFKDERRNKVTFLSSATTALSMQNNSVFGDLKSDEMELLYSAYGDETGVQCALSLQEFVKDAGSYSKKVVDDLLDQITGGDHSRTLFQLKQRRNVPMKPPDEAKVGDTLGDSSSSVLEFMSMKSYPDVSVDISMLSSLGKVKKELDPDDSHLNLDETTKLLQDLHEAQAERGGSRPSSNLSSLSNASERDQHHLGSPSRLSVGEQPDVTHDPYEFLQSPEPAASAKT